(19) United States
(12) Patent Application Publication
(10) Pub. No.: US 2026/0365670 A1
(43) Pub. Date: Nov. 19, 2026

(54) EGFR INHIBITORS AND USES THEREOF

(71) Applicant: DIZAL (JIANGSU) PHARMACEUTICAL CO., LTD., Shanghai (CN)

(72) Inventors: Qingbei ZENG, Shanghai (CN); Honchung TSUI, Shanghai (CN); Zhenfan YANG, Shanghai (CN); Xiaolin ZHANG, Shanghai (CN)

(21) Appl. No.: 19/107,192

(22) PCT Filed: Aug. 25, 2023
(86) PCT No.: PCT/CN2023/114848

§ 371 Date: Feb. 27, 2025

(30) Foreign Application Priority Data
Sep. 2, 2022 (WO) PCT/CN2022/116914
Jul. 13, 2023 (WO) PCT/CN2023/107228

(51) Int. Cl. C07D 498/22; A61K 31/439; A61K 31/4545; A61K 31/4748; A61K 31/496; A61K 31/504; A61K 31/5377; A61K 31/5386; A61K 31/55; A61K 31/553; A61K 45/06; C07D 498/18; C07D 519/00

(52) U.S. Cl. CPC C07D 498/22; A61K 31/439; A61K 31/4545; A61K 31/4748; A61K 31/496; A61K 31/504; A61K 31/5377; A61K 31/5386; A61K 31/55; A61K 31/553; A61K 45/06; C07D 498/18; C07D 519/00

(57) ABSTRACT

Disclosed herein are compounds or pharmaceutically acceptable salts thereof that are useful as EGFR inhibitors. Also disclosed are pharmaceutical compositions comprising such compounds, and methods of using such compounds or compositions to treat EGFR-related disorder (e.g., cancers).

### FIELD OF THE DISCLOSURE

The present disclosure generally relates to novel compounds inhibiting the epidermal growth factor receptor (EGFR), and pharmaceutically acceptable salts thereof. The present disclosure also relates to pharmaceutical compositions comprising the compound as an active ingredient and use of the compounds in the treatment of EGFR-related disorder, including cancers.

### BACKGROUND

The epidermal growth factor receptor (EGFR) is a transmembrane protein that is a receptor for members of the epidermal growth factor family (EGF family) of extracellular protein ligands. EGFR family of receptor tyrosine kinases regulate cell proliferation, survival, adhesion, migration and differentiation. Inhibition of EGFR activity has demonstrated potential therapeutic applicability in a wide range of pathological conditions. Certain cancers are characterized by mutations of EGFR, which results in increased cell proliferation.

Currently available EGFR inhibitors include, for example, gefitinib and erlotinib as the 1^{st} generation EGFR inhibitors and afatinib as the 2^{nd} generation covalent EGFR. Recently, the 3^{rd} generation wild type sparing EGFR inhibitor such as WZ4002 and development of the 4^{th} generation EGFR inhibitors are reported.

There still remains a need for novel compounds inhibiting EGFR, which can be used as pharmacological tools and are of considerable interest as drugs for treating EGFR-related disorders such as cancers.

### SUMMARY OF THE DISCLOSURE

Disclosed herein are novel compounds that are capable of inhibiting EGFR. As a result, the compounds of the present disclosure are useful in the treatment of EGFR-related diseases such as cancers.

In one aspect, the present disclosure provides a compound of Formula (I):

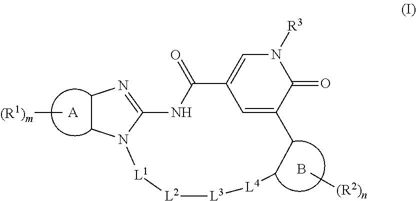

or a pharmaceutically acceptable salt thereof, wherein:
Ring A is selected from the group consisting of cycloalkyl, heterocyclyl, aryl and heteroaryl;
Ring B is selected from the group consisting of cycloalkyl, heterocyclyl, aryl, and heteroaryl;
L¹ is selected from the group consisting of a bond, alkyl, alkenyl, alkynyl, heteroalkyl, heteroalkenyl and heteroalkynyl, wherein the alkyl, alkenyl, alkynyl, heteroalkyl, heteroalkenyl and heteroalkynyl are optionally substituted with one or more groups independently selected from hydroxyl, halogen, cyano and amino;
L² is selected from the group consisting of a bond, N(R^{A}), alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more R^{B};
wherein R^{A} is selected from a group consisting of hydrogen, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, amino, alkyl and haloalkyl;
wherein each R^{B} is independently selected from a group consisting of hydroxyl, halogen, cyano, amino, alkyl, alkenyl, alkynyl, heteroalkyl, heteroalkenyl, cycloalkyl, heterocyclyl, aryl and heteroaryl;
L³ is selected from the group consisting of a bond, alkyl, alkenyl, alkynyl, heteroalkyl, heteroalkenyl and heteroalkynyl, wherein the alkyl, alkenyl, alkynyl, heteroalkyl, heteroalkenyl and heteroalkynyl are optionally substituted with one or more groups independently selected from hydroxyl, halogen, cyano and amino;
L⁴ is selected from O, S, or N(R^{C});
wherein R^{C} is selected from a group consisting of hydrogen, alkyl, alkenyl, alkynyl, haloalkyl, heteroalkyl, heteroalkenyl, heteroalkynyl, cycloalkyl, heterocyclyl, aryl, and heteroaryl;
each R¹ is independently selected from the group consisting of hydroxy, halogen, cyano, amino, —N(R^{D})₂, alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more R^{E};
wherein each R^{D} is independently selected from a group consisting of hydrogen, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, —N(R^{F})₂ or —OR^{G};
wherein each R^{E} is independently selected from a group consisting of hydrogen, hydroxyl, halogen, cyano, amino, —N(R^{F})₂, -alkyl-N(R^{F})₂, —C(O)OR^{G}, alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, amino and alkyl;
wherein each of R^{F} and R^{G} is independently selected from a group consisting of alkyl, alkenyl, alkynyl, heteroalkyl, heteroalkenyl, heteroalkynyl, cycloalkyl, heterocyclyl, aryl and heteroaryl;
each R² is independently selected from the group consisting of hydrogen, hydroxy, halogen, cyano, amino, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, amino, alkyl and haloalkyl;
R³ is selected from the group consisting of hydrogen, alkyl, alkenyl, alkynyl, heteroalkyl, heteroalkenyl, heteroalkynyl, cycloalkyl and heterocyclyl, wherein the alkyl, alkenyl, alkynyl, heteroalkyl, heteroalkenyl, heteroalkynyl, cycloalkyl and heterocyclyl are optionally substituted with one or more groups independently selected from hydroxyl, halogen, cyano, amino, alkyl and haloalkyl;
m is an integer from 0 to 5; and
n is an integer from 0 to 4.

In another aspect, the present disclosure provides a compound or a pharmaceutically acceptable salt thereof, wherein the compound is seleted from a group consisting of:

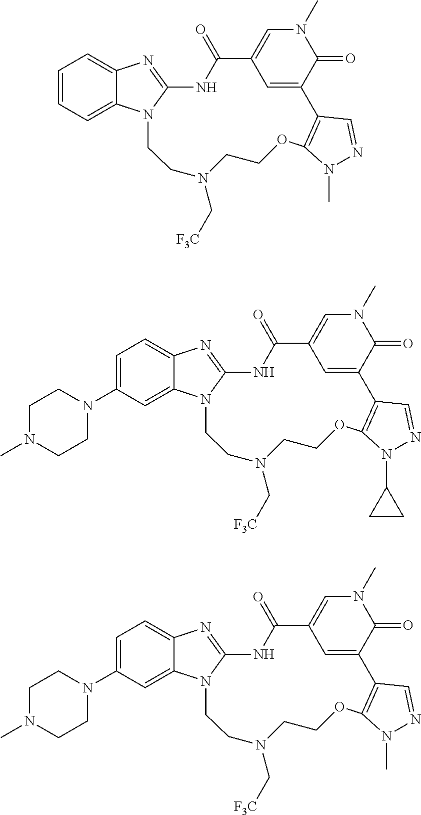 [SMILES: Cn1ncc2c1OCCN(CC(F)(F)F)CCn1c(nc3ccccc31)NC(=O)c1cc-2c(=O)n(C)c1 ; CN1CCN(c2ccc3nc4n(c3c2)CCN(CC(F)(F)F)CCOc2c(cnn2C2CC2)-c2cc(cn(C)c2=O)C(=O)N4)CC1 ; CN1CCN(c2ccc3nc4n(c3c2)CCN(CC(F)(F)F)CCOc2c(cnn2C)-c2cc(cn(C)c2=O)C(=O)N4)CC1]

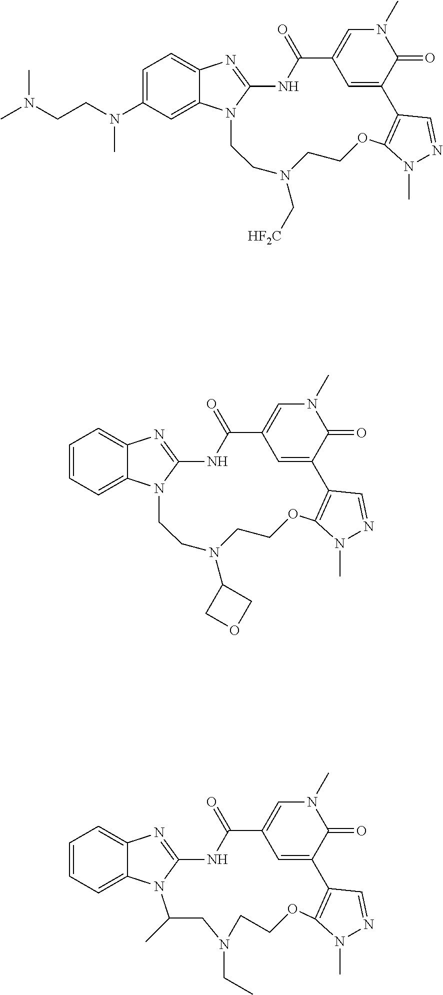

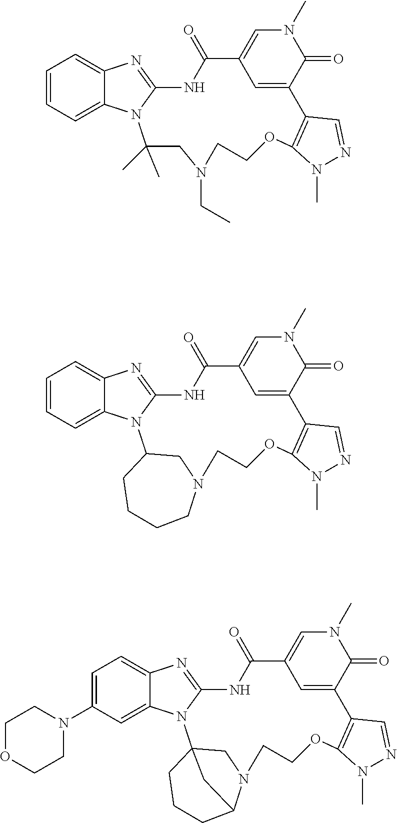 [SMILES: CCN1CCOc2c(cnn2C)-c2cc(cn(C)c2=O)C(=O)Nc2nc3ccccc3n2C(C)(C)C1 ; Cn1ncc2c1OCCN1CCCCC(C1)n1c(nc3ccccc31)NC(=O)c1cc-2c(=O)n(C)c1 ; Cn1ncc2c1OCCN1CC3(CCCC1C3)n1c(nc3ccc(N4CCOCC4)cc31)NC(=O)c1cc-2c(=O)n(C)c1]

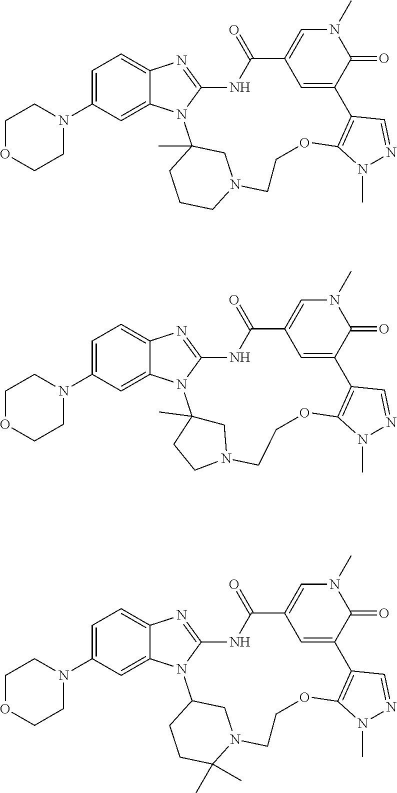 [SMILES: Cn1ncc2c1OCCN1CCCC(C)(C1)n1c(nc3ccc(N4CCOCC4)cc31)NC(=O)c1cc-2c(=O)n(C)c1 ; Cn1ncc2c1OCCN1CCC(C)(C1)n1c(nc3ccc(N4CCOCC4)cc31)NC(=O)c1cc-2c(=O)n(C)c1 ; Cn1ncc2c1OCCN1CC(CCC1(C)C)n1c(nc3ccc(N4CCOCC4)cc31)NC(=O)c1cc-2c(=O)n(C)c1]

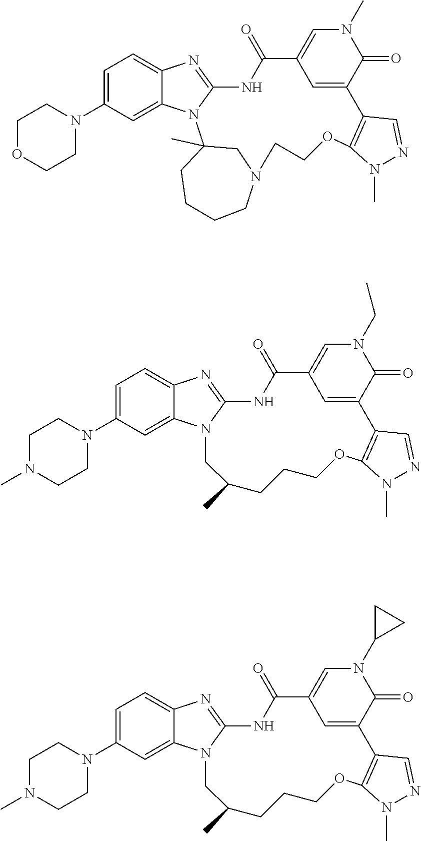 [SMILES: Cn1ncc2c1OCCN1CCCCC(C)(C1)n1c(nc3ccc(N4CCOCC4)cc31)NC(=O)c1cc-2c(=O)n(C)c1 ; CCn1cc2cc(c1=O)-c1cnn(C)c1OCCC[C@@H](C)Cn1c(nc3ccc(N4CCN(C)CC4)cc31)NC2=O ; C[C@@H]1CCCOc2c(cnn2C)-c2cc(cn(C3CC3)c2=O)C(=O)Nc2nc3ccc(N4CCN(C)CC4)cc3n2C1]

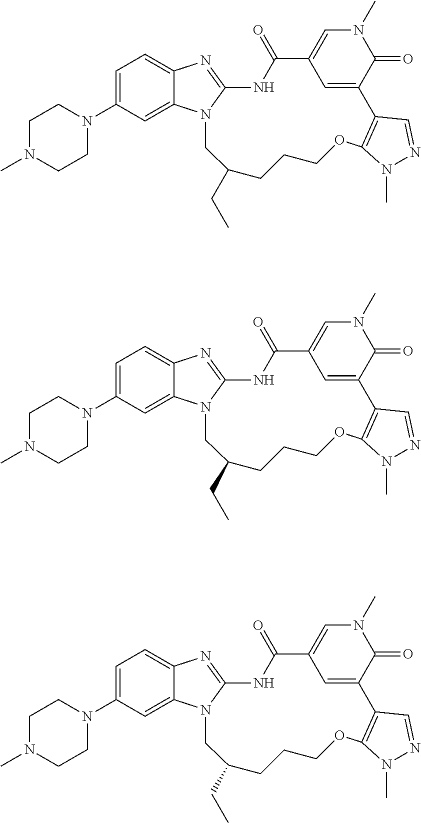 [SMILES: CCC1CCCOc2c(cnn2C)-c2cc(cn(C)c2=O)C(=O)Nc2nc3ccc(N4CCN(C)CC4)cc3n2C1 ; CC[C@@H]1CCCOc2c(cnn2C)-c2cc(cn(C)c2=O)C(=O)Nc2nc3ccc(N4CCN(C)CC4)cc3n2C1 ; CC[C@H]1CCCOc2c(cnn2C)-c2cc(cn(C)c2=O)C(=O)Nc2nc3ccc(N4CCN(C)CC4)cc3n2C1]

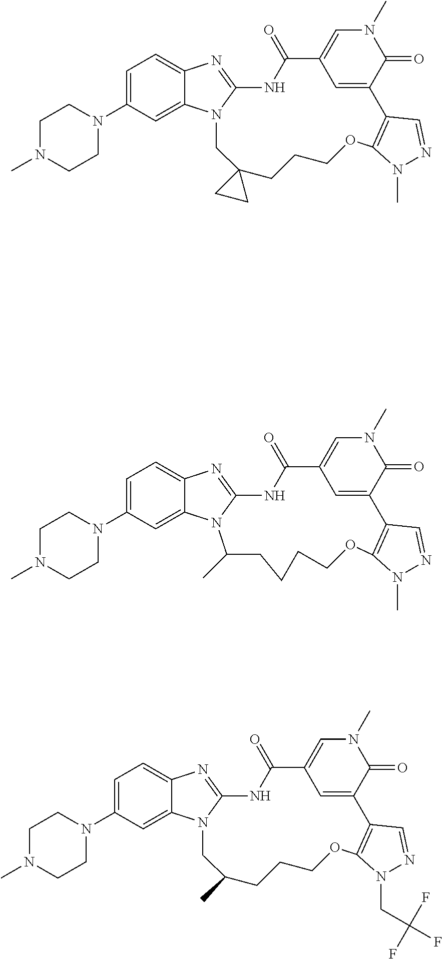 [SMILES: CN1CCN(c2ccc3nc4n(c3c2)CC2(CCCOc3c(cnn3C)-c3cc(cn(C)c3=O)C(=O)N4)CC2)CC1 ; CC1CCCCOc2c(cnn2C)-c2cc(cn(C)c2=O)C(=O)Nc2nc3ccc(N4CCN(C)CC4)cc3n21 ; C[C@@H]1CCCOc2c(cnn2CC(F)(F)F)-c2cc(cn(C)c2=O)C(=O)Nc2nc3ccc(N4CCN(C)CC4)cc3n2C1]

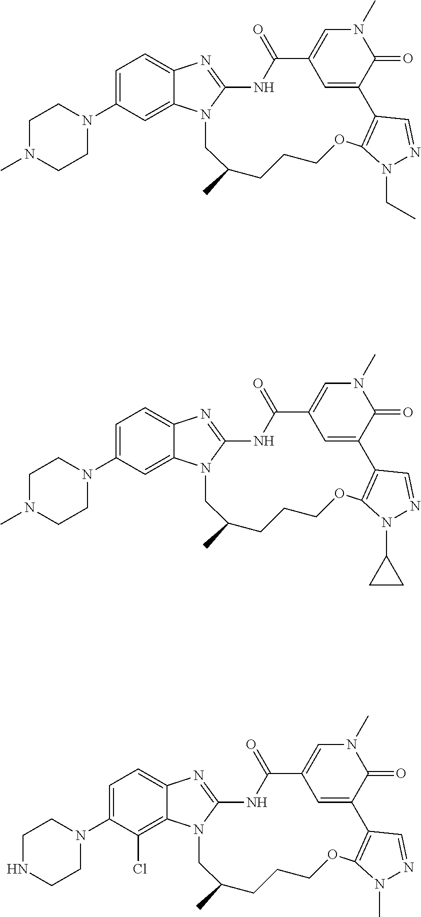

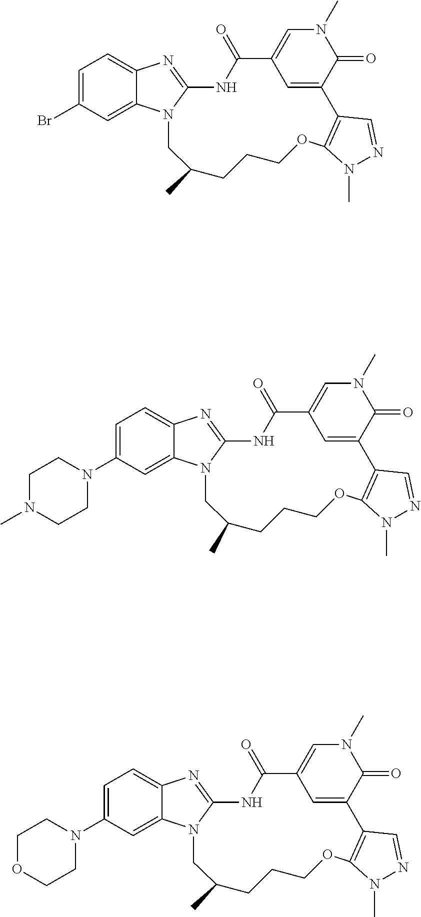 [SMILES: C[C@@H]1CCCOc2c(cnn2C)-c2cc(cn(C)c2=O)C(=O)Nc2nc3ccc(Br)cc3n2C1 ; C[C@@H]1CCCOc2c(cnn2C)-c2cc(cn(C)c2=O)C(=O)Nc2nc3ccc(N4CCN(C)CC4)cc3n2C1 ; C[C@@H]1CCCOc2c(cnn2C)-c2cc(cn(C)c2=O)C(=O)Nc2nc3ccc(N4CCOCC4)cc3n2C1]

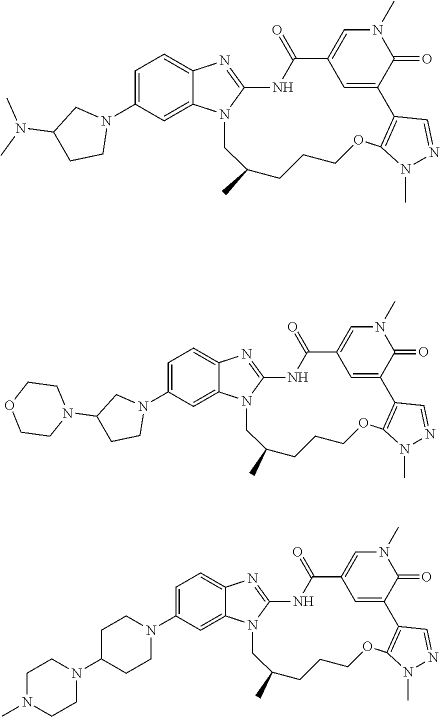 [SMILES: C[C@@H]1CCCOc2c(cnn2C)-c2cc(cn(C)c2=O)C(=O)Nc2nc3ccc(N4CCC(N(C)C)C4)cc3n2C1 ; C[C@@H]1CCCOc2c(cnn2C)-c2cc(cn(C)c2=O)C(=O)Nc2nc3ccc(N4CCC(N5CCOCC5)C4)cc3n2C1 ; C[C@@H]1CCCOc2c(cnn2C)-c2cc(cn(C)c2=O)C(=O)Nc2nc3ccc(N4CCC(N5CCN(C)CC5)CC4)cc3n2C1]

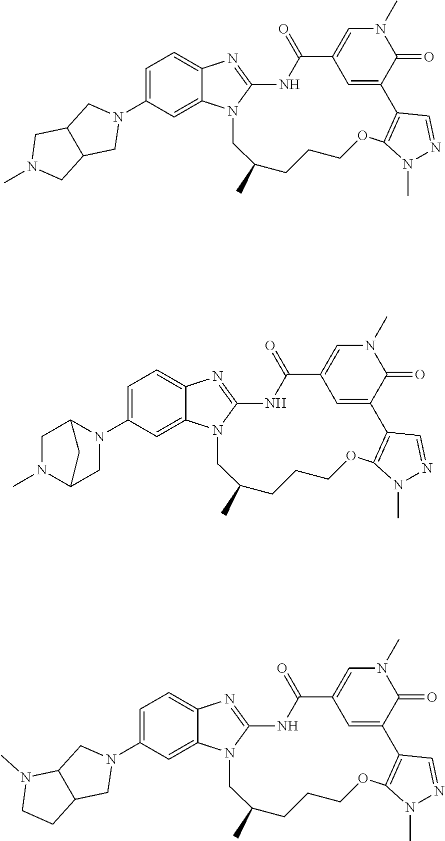 [SMILES: C[C@@H]1CCCOc2c(cnn2C)-c2cc(cn(C)c2=O)C(=O)Nc2nc3ccc(N4CC5CN(C)CC5C4)cc3n2C1 ; C[C@@H]1CCCOc2c(cnn2C)-c2cc(cn(C)c2=O)C(=O)Nc2nc3ccc(N4CC5CC4CN5C)cc3n2C1 ; C[C@@H]1CCCOc2c(cnn2C)-c2cc(cn(C)c2=O)C(=O)Nc2nc3ccc(N4CC5CCN(C)C5C4)cc3n2C1]

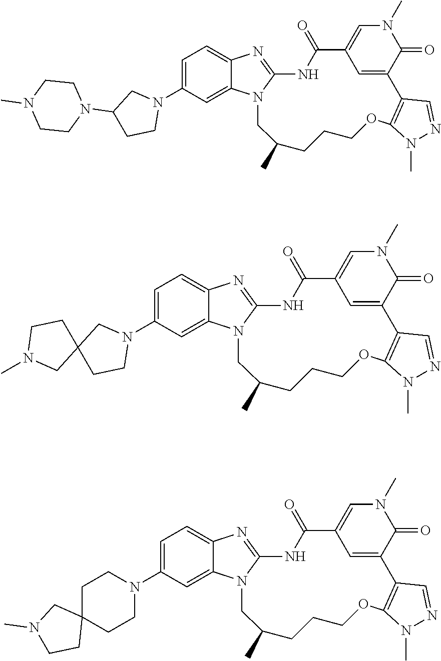 [SMILES: C[C@@H]1CCCOc2c(cnn2C)-c2cc(cn(C)c2=O)C(=O)Nc2nc3ccc(N4CCC(N5CCN(C)CC5)C4)cc3n2C1 ; C[C@@H]1CCCOc2c(cnn2C)-c2cc(cn(C)c2=O)C(=O)Nc2nc3ccc(N4CCC5(CCN(C)C5)C4)cc3n2C1 ; C[C@@H]1CCCOc2c(cnn2C)-c2cc(cn(C)c2=O)C(=O)Nc2nc3ccc(N4CCC5(CCN(C)C5)CC4)cc3n2C1]

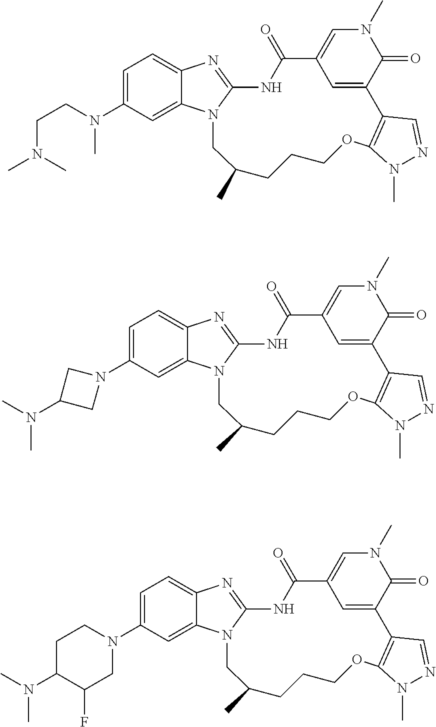 [SMILES: C[C@@H]1CCCOc2c(cnn2C)-c2cc(cn(C)c2=O)C(=O)Nc2nc3ccc(N(C)CCN(C)C)cc3n2C1 ; C[C@@H]1CCCOc2c(cnn2C)-c2cc(cn(C)c2=O)C(=O)Nc2nc3ccc(N4CC(N(C)C)C4)cc3n2C1 ; C[C@@H]1CCCOc2c(cnn2C)-c2cc(cn(C)c2=O)C(=O)Nc2nc3ccc(N4CCC(N(C)C)C(F)C4)cc3n2C1]

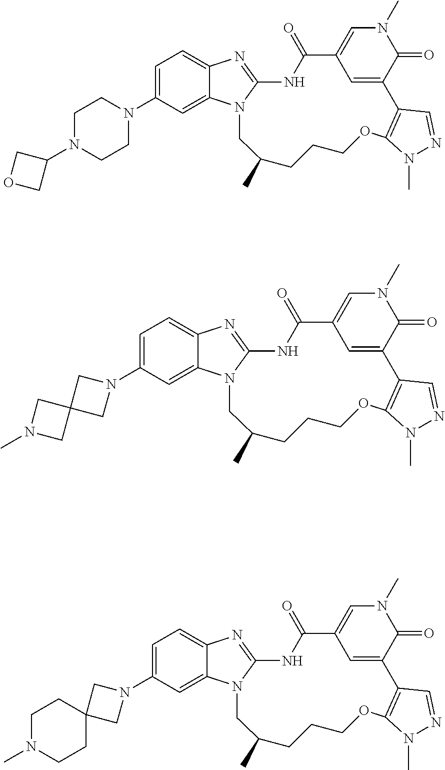 [SMILES: C[C@@H]1CCCOc2c(cnn2C)-c2cc(cn(C)c2=O)C(=O)Nc2nc3ccc(N4CCN(C5COC5)CC4)cc3n2C1 ; C[C@@H]1CCCOc2c(cnn2C)-c2cc(cn(C)c2=O)C(=O)Nc2nc3ccc(N4CC5(CN(C)C5)C4)cc3n2C1 ; C[C@@H]1CCCOc2c(cnn2C)-c2cc(cn(C)c2=O)C(=O)Nc2nc3ccc(N4CC5(CCN(C)CC5)C4)cc3n2C1]

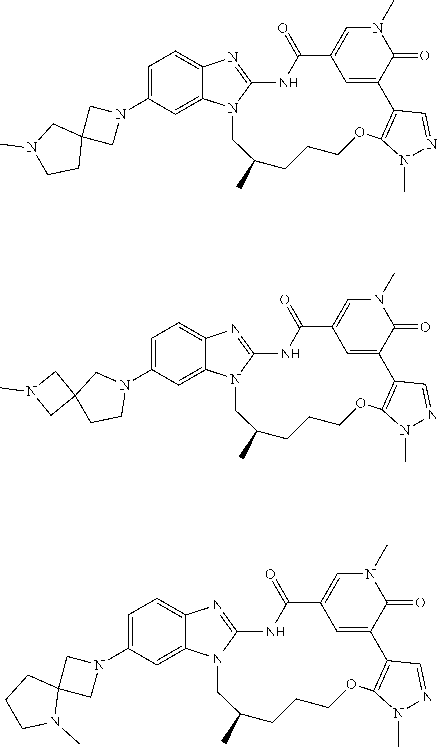 [SMILES: C[C@@H]1CCCOc2c(cnn2C)-c2cc(cn(C)c2=O)C(=O)Nc2nc3ccc(N4CC5(CCN(C)C5)C4)cc3n2C1 ; C[C@@H]1CCCOc2c(cnn2C)-c2cc(cn(C)c2=O)C(=O)Nc2nc3ccc(N4CCC5(CN(C)C5)C4)cc3n2C1 ; C[C@@H]1CCCOc2c(cnn2C)-c2cc(cn(C)c2=O)C(=O)Nc2nc3ccc(N4CC5(CCCN5C)C4)cc3n2C1]

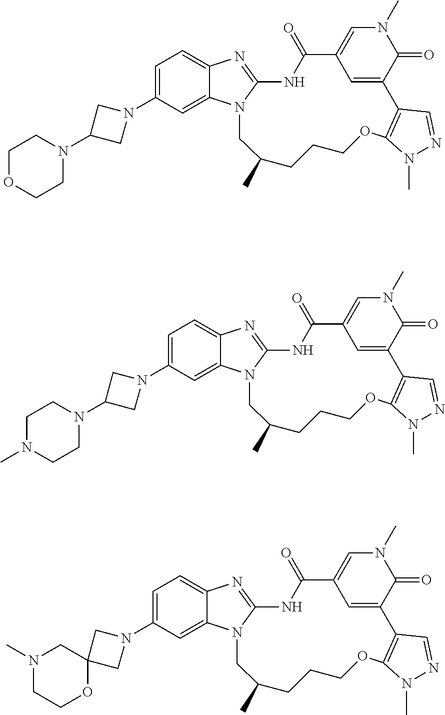 [SMILES: C[C@@H]1CCCOc2c(cnn2C)-c2cc(cn(C)c2=O)C(=O)Nc2nc3ccc(N4CC(N5CCOCC5)C4)cc3n2C1 ; C[C@@H]1CCCOc2c(cnn2C)-c2cc(cn(C)c2=O)C(=O)Nc2nc3ccc(N4CC(N5CCN(C)CC5)C4)cc3n2C1 ; C[C@@H]1CCCOc2c(cnn2C)-c2cc(cn(C)c2=O)C(=O)Nc2nc3ccc(N4CC5(CN(C)CCO5)C4)cc3n2C1]

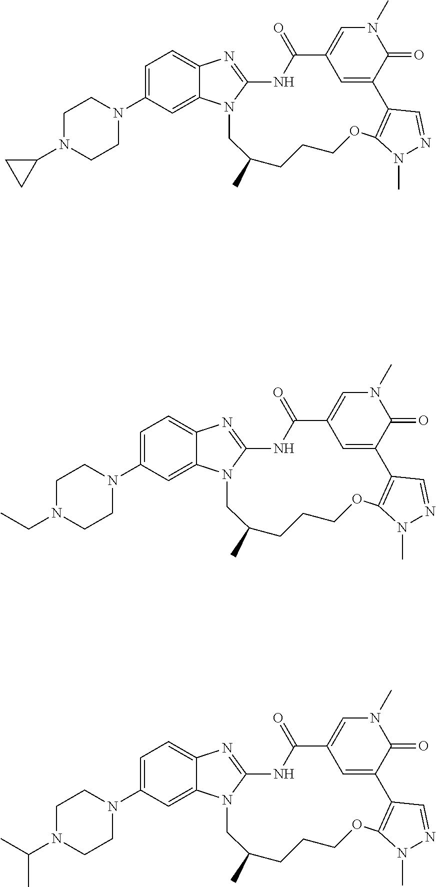 [SMILES: C[C@@H]1CCCOc2c(cnn2C)-c2cc(cn(C)c2=O)C(=O)Nc2nc3ccc(N4CCN(C5CC5)CC4)cc3n2C1 ; CCN1CCN(c2ccc3nc4n(c3c2)C[C@H](C)CCCOc2c(cnn2C)-c2cc(cn(C)c2=O)C(=O)N4)CC1 ; CC(C)N1CCN(c2ccc3nc4n(c3c2)C[C@H](C)CCCOc2c(cnn2C)-c2cc(cn(C)c2=O)C(=O)N4)CC1]

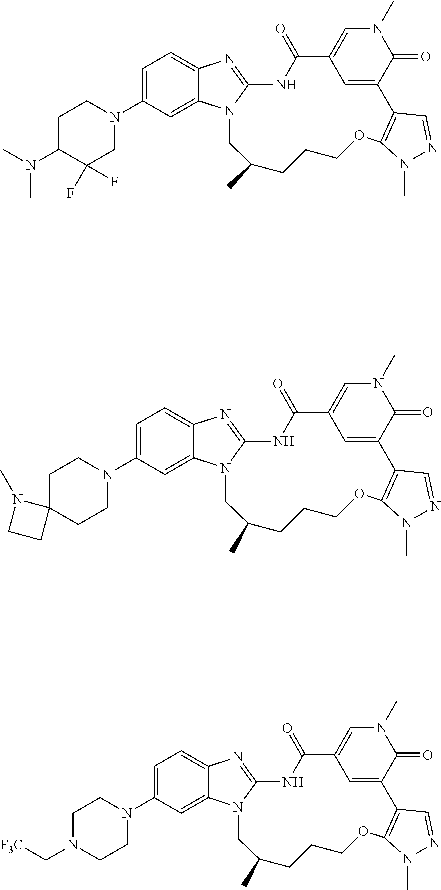 [SMILES: C[C@@H]1CCCOc2c(cnn2C)-c2cc(cn(C)c2=O)C(=O)Nc2nc3ccc(N4CCC(N(C)C)C(F)(F)C4)cc3n2C1 ; C[C@@H]1CCCOc2c(cnn2C)-c2cc(cn(C)c2=O)C(=O)Nc2nc3ccc(N4CCC5(CC4)CCN5C)cc3n2C1 ; C[C@@H]1CCCOc2c(cnn2C)-c2cc(cn(C)c2=O)C(=O)Nc2nc3ccc(N4CCN(CC(F)(F)F)CC4)cc3n2C1]

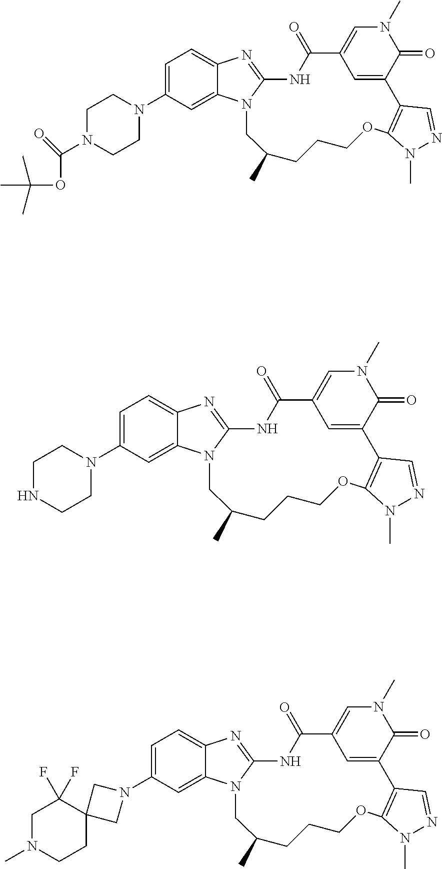 [SMILES: C[C@@H]1CCCOc2c(cnn2C)-c2cc(cn(C)c2=O)C(=O)Nc2nc3ccc(N4CCN(C(=O)OC(C)(C)C)CC4)cc3n2C1 ; C[C@@H]1CCCOc2c(cnn2C)-c2cc(cn(C)c2=O)C(=O)Nc2nc3ccc(N4CCNCC4)cc3n2C1 ; C[C@@H]1CCCOc2c(cnn2C)-c2cc(cn(C)c2=O)C(=O)Nc2nc3ccc(N4CC5(CCN(C)CC5(F)F)C4)cc3n2C1]

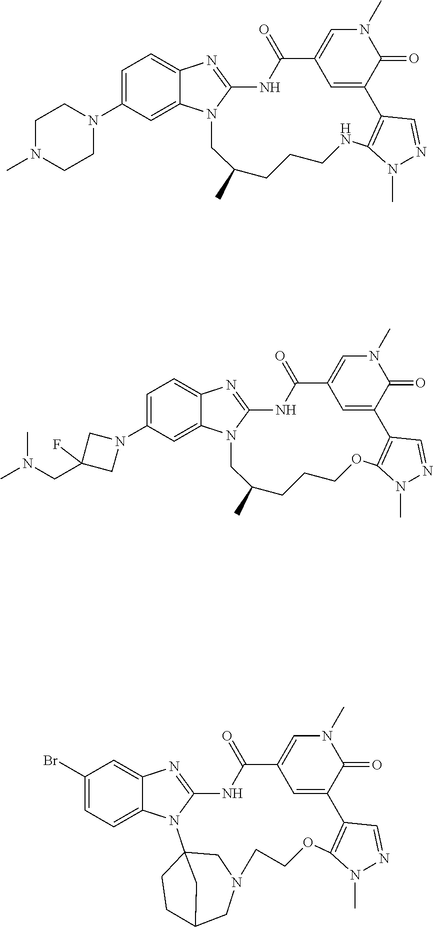 [SMILES: C[C@@H]1CCCNc2c(cnn2C)-c2cc(cn(C)c2=O)C(=O)Nc2nc3ccc(N4CCN(C)CC4)cc3n2C1 ; C[C@@H]1CCCOc2c(cnn2C)-c2cc(cn(C)c2=O)C(=O)Nc2nc3ccc(N4CC(F)(CN(C)C)C4)cc3n2C1 ; Cn1ncc2c1OCCN1CC3CCC(C3)(C1)n1c(nc3cc(Br)ccc31)NC(=O)c1cc-2c(=O)n(C)c1]

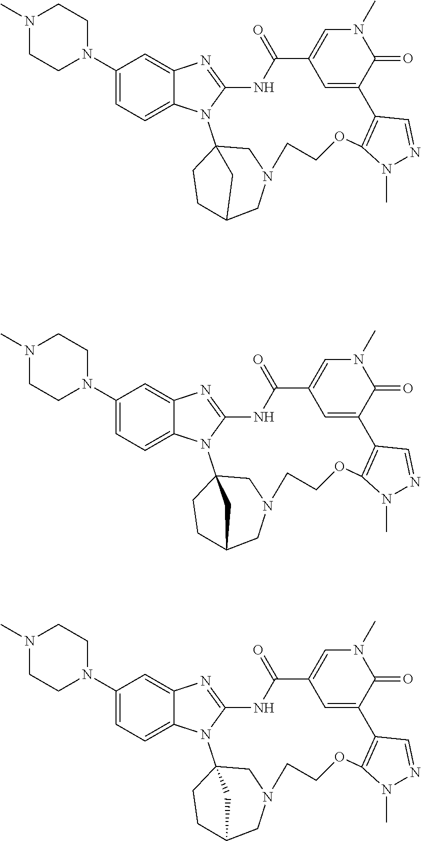 [SMILES: CN1CCN(c2ccc3c(c2)nc2n3C34CCC(CN(CCOc5c(cnn5C)-c5cc(cn(C)c5=O)C(=O)N2)C3)C4)CC1 ; CN1CCN(c2ccc3c(c2)nc2n3[C@@]34CC[C@@H](CN(CCOc5c(cnn5C)-c5cc(cn(C)c5=O)C(=O)N2)C3)C4)CC1 ; CN1CCN(c2ccc3c(c2)nc2n3[C@]34CC[C@H](CN(CCOc5c(cnn5C)-c5cc(cn(C)c5=O)C(=O)N2)C3)C4)CC1]

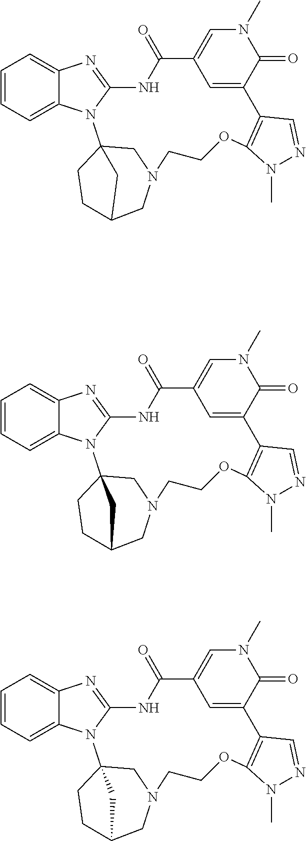 [SMILES: Cn1ncc2c1OCCN1CC3CCC(C3)(C1)n1c(nc3ccccc31)NC(=O)c1cc-2c(=O)n(C)c1 ; Cn1ncc2c1OCCN1C[C@@H]3CC[C@@](C3)(C1)n1c(nc3ccccc31)NC(=O)c1cc-2c(=O)n(C)c1 ; Cn1ncc2c1OCCN1C[C@H]3CC[C@](C3)(C1)n1c(nc3ccccc31)NC(=O)c1cc-2c(=O)n(C)c1]

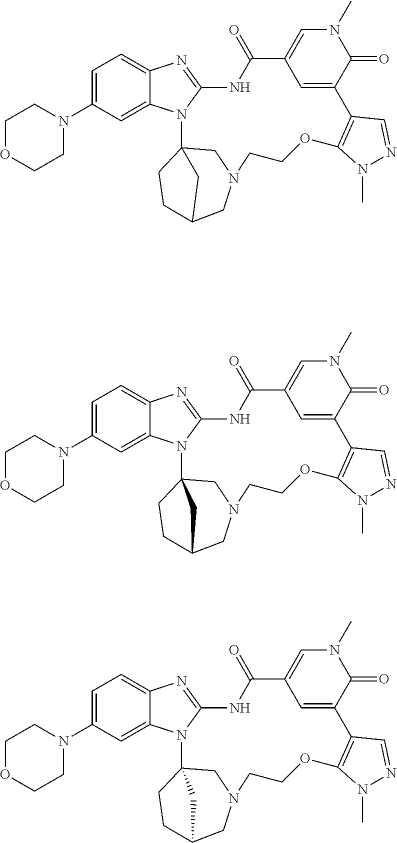 [SMILES: Cn1ncc2c1OCCN1CC3CCC(C3)(C1)n1c(nc3ccc(N4CCOCC4)cc31)NC(=O)c1cc-2c(=O)n(C)c1 ; Cn1ncc2c1OCCN1C[C@@H]3CC[C@@](C3)(C1)n1c(nc3ccc(N4CCOCC4)cc31)NC(=O)c1cc-2c(=O)n(C)c1 ; Cn1ncc2c1OCCN1C[C@H]3CC[C@](C3)(C1)n1c(nc3ccc(N4CCOCC4)cc31)NC(=O)c1cc-2c(=O)n(C)c1]

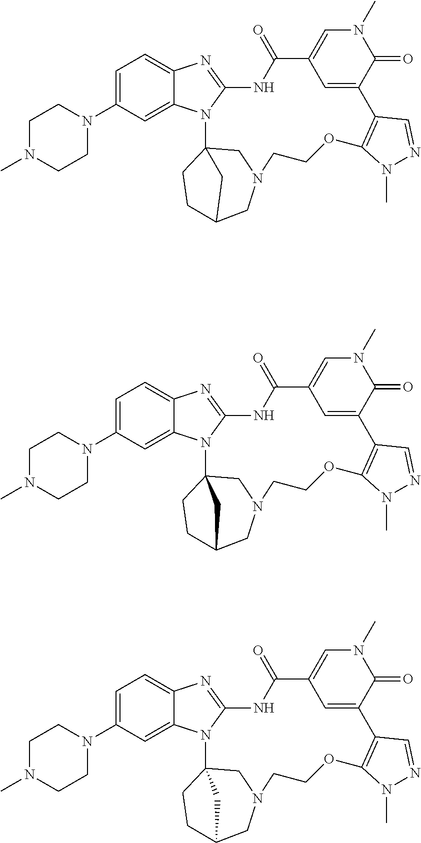 [SMILES: CN1CCN(c2ccc3nc4n(c3c2)C23CCC(CN(CCOc5c(cnn5C)-c5cc(cn(C)c5=O)C(=O)N4)C2)C3)CC1 ; CN1CCN(c2ccc3nc4n(c3c2)[C@@]23CC[C@@H](CN(CCOc5c(cnn5C)-c5cc(cn(C)c5=O)C(=O)N4)C2)C3)CC1 ; CN1CCN(c2ccc3nc4n(c3c2)[C@]23CC[C@H](CN(CCOc5c(cnn5C)-c5cc(cn(C)c5=O)C(=O)N4)C2)C3)CC1]

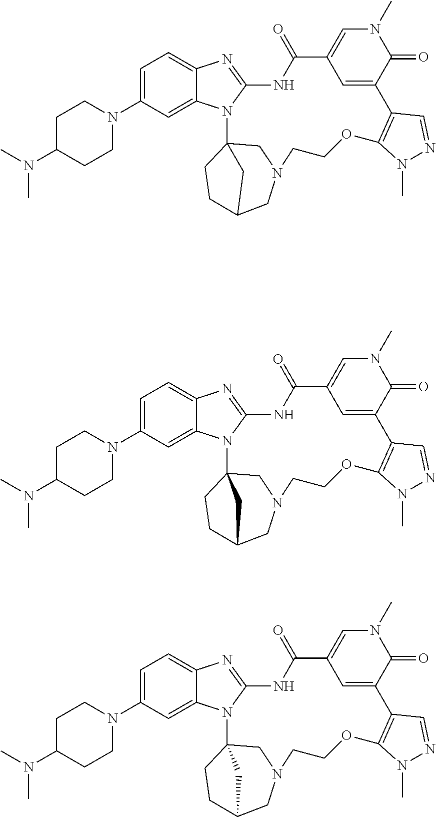 [SMILES: CN(C)C1CCN(c2ccc3nc4n(c3c2)C23CCC(CN(CCOc5c(cnn5C)-c5cc(cn(C)c5=O)C(=O)N4)C2)C3)CC1 ; CN(C)C1CCN(c2ccc3nc4n(c3c2)[C@@]23CC[C@@H](CN(CCOc5c(cnn5C)-c5cc(cn(C)c5=O)C(=O)N4)C2)C3)CC1 ; CN(C)C1CCN(c2ccc3nc4n(c3c2)[C@]23CC[C@H](CN(CCOc5c(cnn5C)-c5cc(cn(C)c5=O)C(=O)N4)C2)C3)CC1]

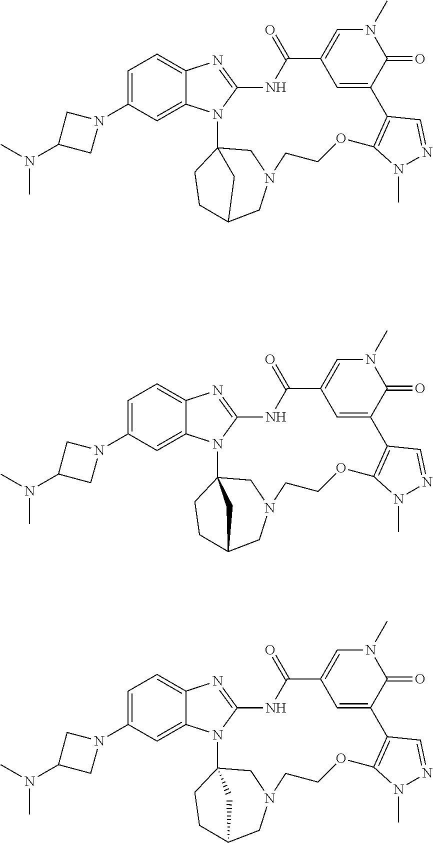 [SMILES: CN(C)C1CN(c2ccc3nc4n(c3c2)C23CCC(CN(CCOc5c(cnn5C)-c5cc(cn(C)c5=O)C(=O)N4)C2)C3)C1 ; CN(C)C1CN(c2ccc3nc4n(c3c2)[C@@]23CC[C@@H](CN(CCOc5c(cnn5C)-c5cc(cn(C)c5=O)C(=O)N4)C2)C3)C1 ; CN(C)C1CN(c2ccc3nc4n(c3c2)[C@]23CC[C@H](CN(CCOc5c(cnn5C)-c5cc(cn(C)c5=O)C(=O)N4)C2)C3)C1]

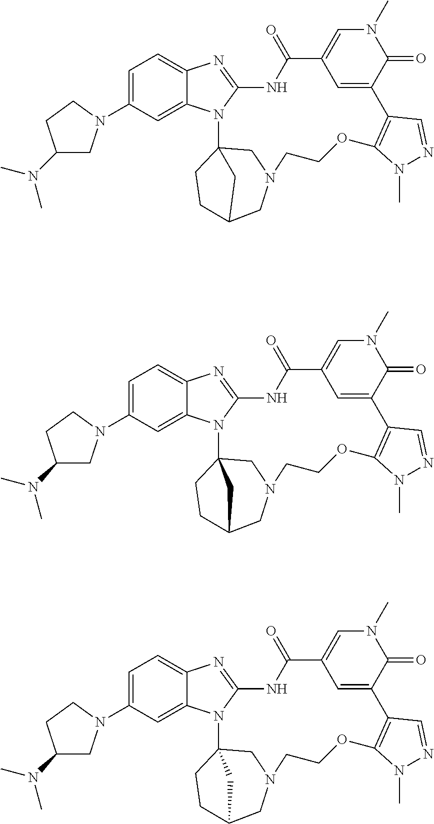 [SMILES: CN(C)C1CCN(c2ccc3nc4n(c3c2)C23CCC(CN(CCOc5c(cnn5C)-c5cc(cn(C)c5=O)C(=O)N4)C2)C3)C1 ; CN(C)[C@H]1CCN(c2ccc3nc4n(c3c2)[C@@]23CC[C@@H](CN(CCOc5c(cnn5C)-c5cc(cn(C)c5=O)C(=O)N4)C2)C3)C1 ; CN(C)[C@H]1CCN(c2ccc3nc4n(c3c2)[C@]23CC[C@H](CN(CCOc5c(cnn5C)-c5cc(cn(C)c5=O)C(=O)N4)C2)C3)C1]

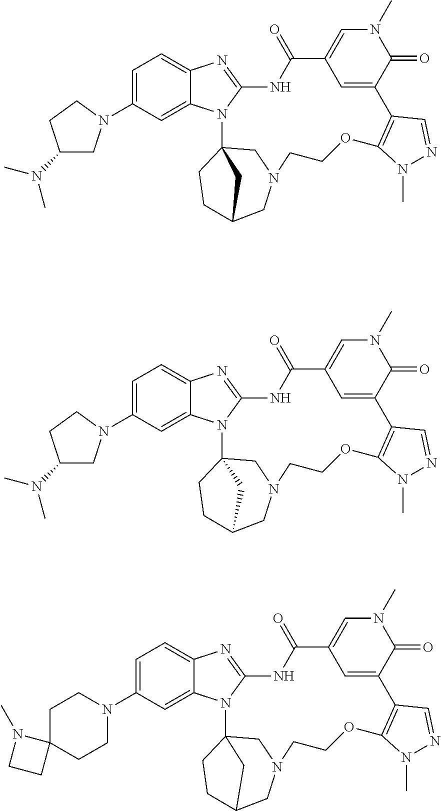 [SMILES: CN(C)[C@@H]1CCN(c2ccc3nc4n(c3c2)[C@@]23CC[C@@H](CN(CCOc5c(cnn5C)-c5cc(cn(C)c5=O)C(=O)N4)C2)C3)C1 ; CN(C)[C@@H]1CCN(c2ccc3nc4n(c3c2)[C@]23CC[C@H](CN(CCOc5c(cnn5C)-c5cc(cn(C)c5=O)C(=O)N4)C2)C3)C1 ; CN1CCC12CCN(c1ccc3nc4n(c3c1)C13CCC(CN(CCOc5c(cnn5C)-c5cc(cn(C)c5=O)C(=O)N4)C1)C3)CC2]

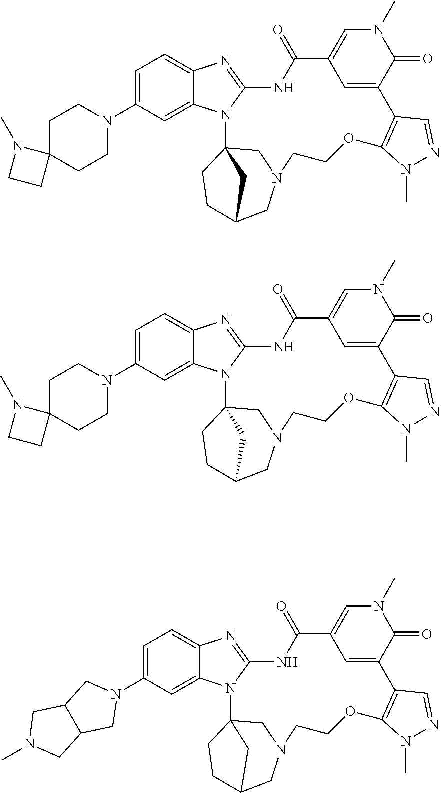 [SMILES: CN1CCC12CCN(c1ccc3nc4n(c3c1)[C@@]13CC[C@@H](CN(CCOc5c(cnn5C)-c5cc(cn(C)c5=O)C(=O)N4)C1)C3)CC2 ; CN1CCC12CCN(c1ccc3nc4n(c3c1)[C@]13CC[C@H](CN(CCOc5c(cnn5C)-c5cc(cn(C)c5=O)C(=O)N4)C1)C3)CC2 ; CN1CC2CN(c3ccc4nc5n(c4c3)C34CCC(CN(CCOc6c(cnn6C)-c6cc(cn(C)c6=O)C(=O)N5)C3)C4)CC2C1]

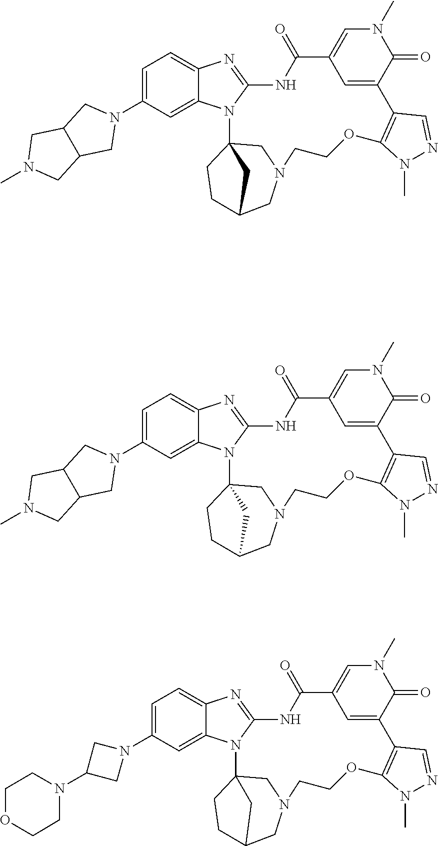 [SMILES: CN1CC2CN(c3ccc4nc5n(c4c3)[C@@]34CC[C@@H](CN(CCOc6c(cnn6C)-c6cc(cn(C)c6=O)C(=O)N5)C3)C4)CC2C1 ; CN1CC2CN(c3ccc4nc5n(c4c3)[C@]34CC[C@H](CN(CCOc6c(cnn6C)-c6cc(cn(C)c6=O)C(=O)N5)C3)C4)CC2C1 ; Cn1ncc2c1OCCN1CC3CCC(C3)(C1)n1c(nc3ccc(N4CC(N5CCOCC5)C4)cc31)NC(=O)c1cc-2c(=O)n(C)c1]

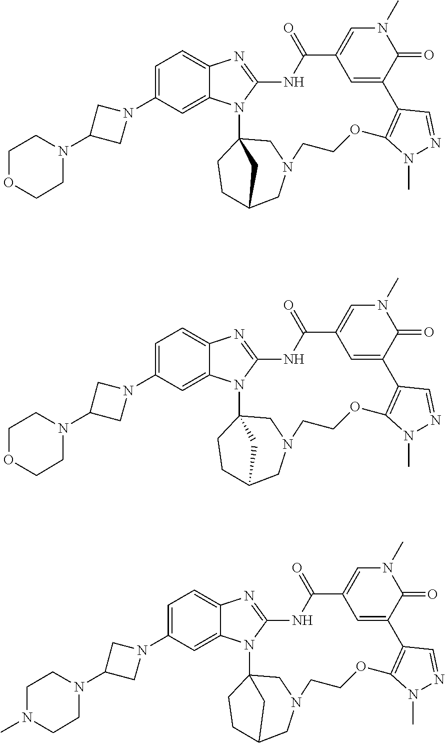 [SMILES: Cn1ncc2c1OCCN1C[C@@H]3CC[C@@](C3)(C1)n1c(nc3ccc(N4CC(N5CCOCC5)C4)cc31)NC(=O)c1cc-2c(=O)n(C)c1 ; Cn1ncc2c1OCCN1C[C@H]3CC[C@](C3)(C1)n1c(nc3ccc(N4CC(N5CCOCC5)C4)cc31)NC(=O)c1cc-2c(=O)n(C)c1 ; CN1CCN(C2CN(c3ccc4nc5n(c4c3)C34CCC(CN(CCOc6c(cnn6C)-c6cc(cn(C)c6=O)C(=O)N5)C3)C4)C2)CC1]

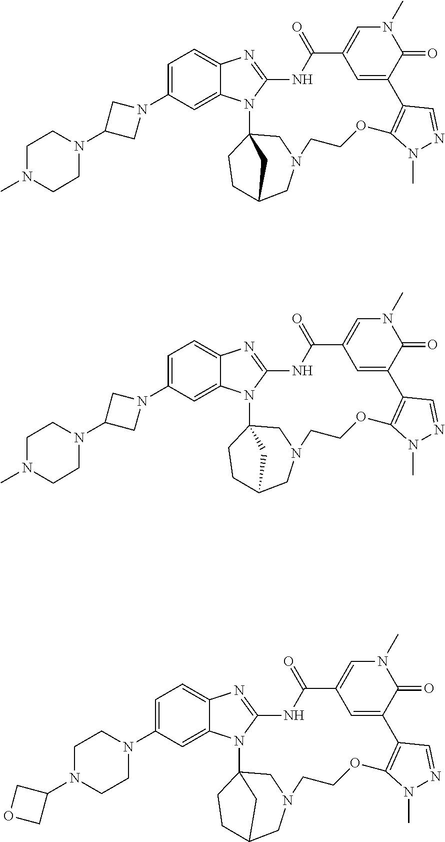 [SMILES: CN1CCN(C2CN(c3ccc4nc5n(c4c3)[C@@]34CC[C@@H](CN(CCOc6c(cnn6C)-c6cc(cn(C)c6=O)C(=O)N5)C3)C4)C2)CC1 ; CN1CCN(C2CN(c3ccc4nc5n(c4c3)[C@]34CC[C@H](CN(CCOc6c(cnn6C)-c6cc(cn(C)c6=O)C(=O)N5)C3)C4)C2)CC1 ; Cn1ncc2c1OCCN1CC3CCC(C3)(C1)n1c(nc3ccc(N4CCN(C5COC5)CC4)cc31)NC(=O)c1cc-2c(=O)n(C)c1]

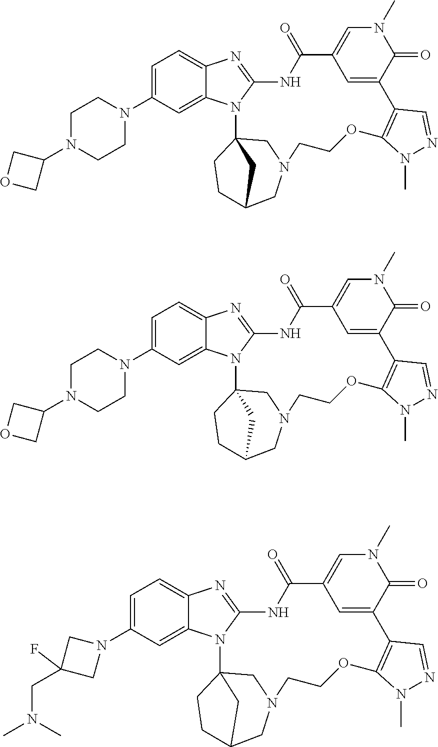 [SMILES: Cn1ncc2c1OCCN1C[C@@H]3CC[C@@](C3)(C1)n1c(nc3ccc(N4CCN(C5COC5)CC4)cc31)NC(=O)c1cc-2c(=O)n(C)c1 ; Cn1ncc2c1OCCN1C[C@H]3CC[C@](C3)(C1)n1c(nc3ccc(N4CCN(C5COC5)CC4)cc31)NC(=O)c1cc-2c(=O)n(C)c1 ; CN(C)CC1(F)CN(c2ccc3nc4n(c3c2)C23CCC(CN(CCOc5c(cnn5C)-c5cc(cn(C)c5=O)C(=O)N4)C2)C3)C1]

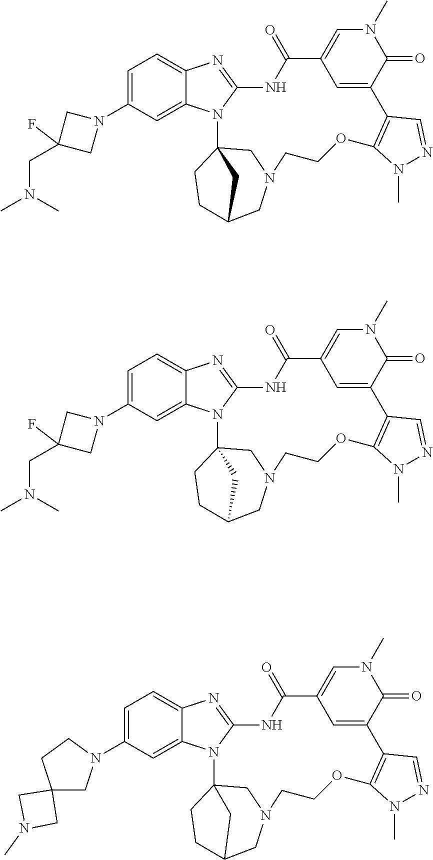 [SMILES: CN(C)CC1(F)CN(c2ccc3nc4n(c3c2)[C@@]23CC[C@@H](CN(CCOc5c(cnn5C)-c5cc(cn(C)c5=O)C(=O)N4)C2)C3)C1 ; CN(C)CC1(F)CN(c2ccc3nc4n(c3c2)[C@]23CC[C@H](CN(CCOc5c(cnn5C)-c5cc(cn(C)c5=O)C(=O)N4)C2)C3)C1 ; CN1CC2(CCN(c3ccc4nc5n(c4c3)C34CCC(CN(CCOc6c(cnn6C)-c6cc(cn(C)c6=O)C(=O)N5)C3)C4)C2)C1]

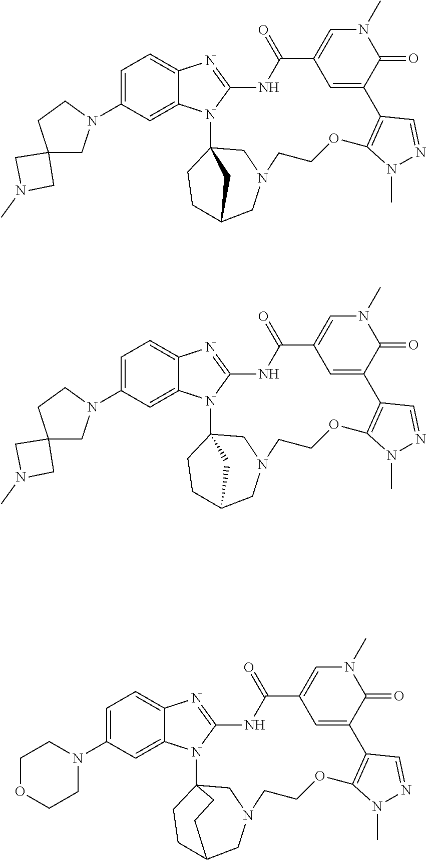 [SMILES: CN1CC2(CCN(c3ccc4nc5n(c4c3)[C@@]34CC[C@@H](CN(CCOc6c(cnn6C)-c6cc(cn(C)c6=O)C(=O)N5)C3)C4)C2)C1 ; CN1CC2(CCN(c3ccc4nc5n(c4c3)[C@]34CC[C@H](CN(CCOc6c(cnn6C)-c6cc(cn(C)c6=O)C(=O)N5)C3)C4)C2)C1 ; Cn1ncc2c1OCCN1CC3CCC(CC3)(C1)n1c(nc3ccc(N4CCOCC4)cc31)NC(=O)c1cc-2c(=O)n(C)c1]

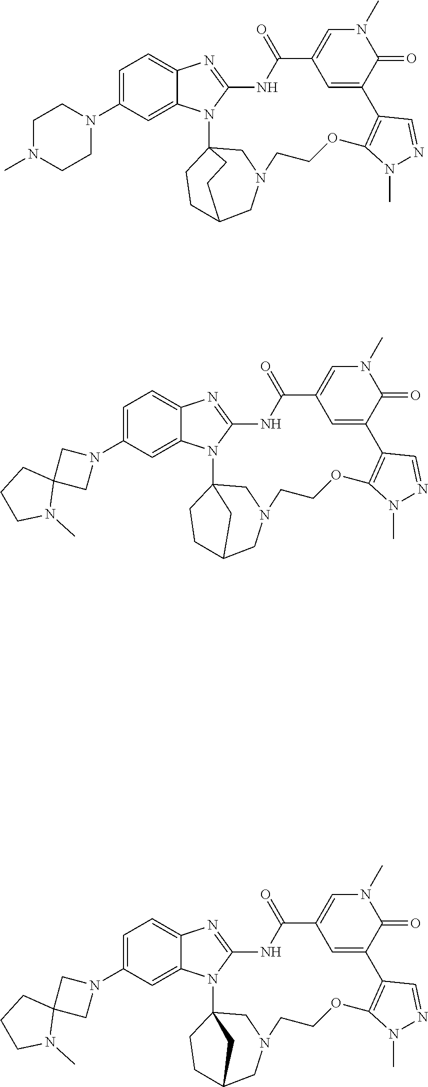 [SMILES: CN1CCN(c2ccc3nc4n(c3c2)C23CCC(CC2)CN(CCOc2c(cnn2C)-c2cc(cn(C)c2=O)C(=O)N4)C3)CC1 ; CN1CCCC12CN(c1ccc3nc4n(c3c1)C13CCC(CN(CCOc5c(cnn5C)-c5cc(cn(C)c5=O)C(=O)N4)C1)C3)C2 ; CN1CCCC12CN(c1ccc3nc4n(c3c1)[C@@]13CC[C@@H](CN(CCOc5c(cnn5C)-c5cc(cn(C)c5=O)C(=O)N4)C1)C3)C2]

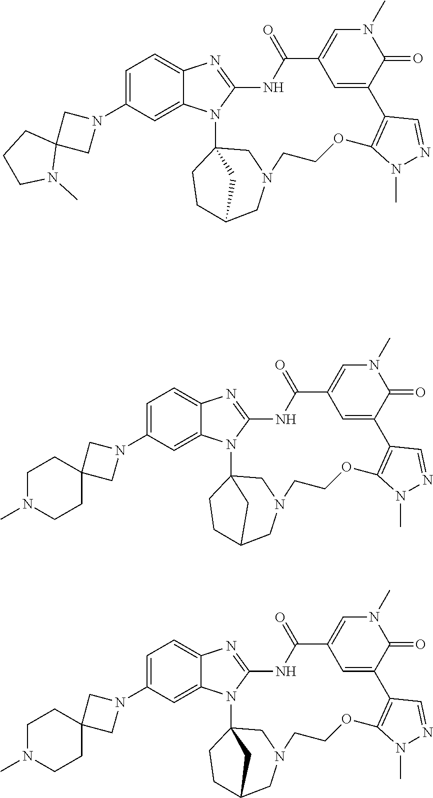 [SMILES: CN1CCCC12CN(c1ccc3nc4n(c3c1)[C@]13CC[C@H](CN(CCOc5c(cnn5C)-c5cc(cn(C)c5=O)C(=O)N4)C1)C3)C2 ; CN1CCC2(CC1)CN(c1ccc3nc4n(c3c1)C13CCC(CN(CCOc5c(cnn5C)-c5cc(cn(C)c5=O)C(=O)N4)C1)C3)C2 ; CN1CCC2(CC1)CN(c1ccc3nc4n(c3c1)[C@@]13CC[C@@H](CN(CCOc5c(cnn5C)-c5cc(cn(C)c5=O)C(=O)N4)C1)C3)C2]

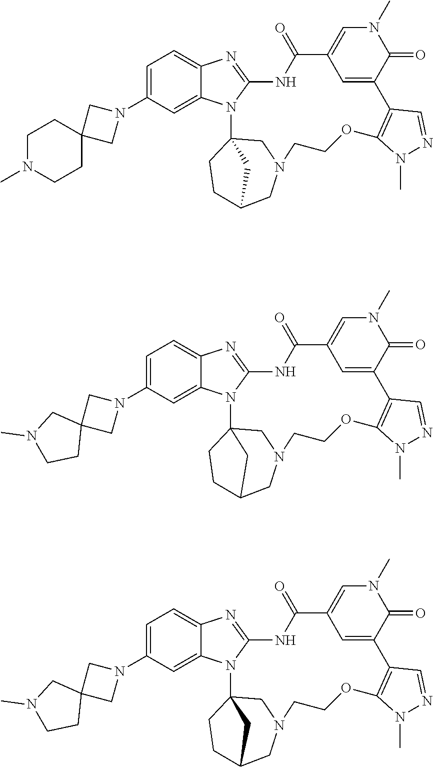 [SMILES: CN1CCC2(CC1)CN(c1ccc3nc4n(c3c1)[C@]13CC[C@H](CN(CCOc5c(cnn5C)-c5cc(cn(C)c5=O)C(=O)N4)C1)C3)C2 ; CN1CCC2(C1)CN(c1ccc3nc4n(c3c1)C13CCC(CN(CCOc5c(cnn5C)-c5cc(cn(C)c5=O)C(=O)N4)C1)C3)C2 ; CN1CCC2(C1)CN(c1ccc3nc4n(c3c1)[C@@]13CC[C@@H](CN(CCOc5c(cnn5C)-c5cc(cn(C)c5=O)C(=O)N4)C1)C3)C2]

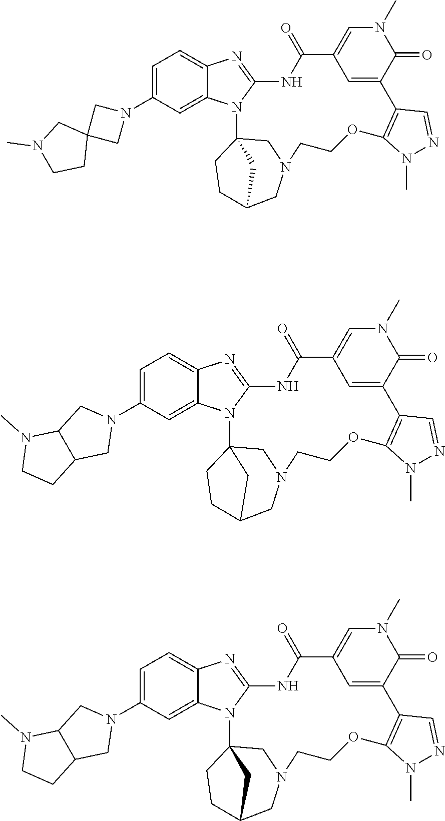 [SMILES: CN1CCC2(C1)CN(c1ccc3nc4n(c3c1)[C@]13CC[C@H](CN(CCOc5c(cnn5C)-c5cc(cn(C)c5=O)C(=O)N4)C1)C3)C2 ; CN1CCC2CN(c3ccc4nc5n(c4c3)C34CCC(CN(CCOc6c(cnn6C)-c6cc(cn(C)c6=O)C(=O)N5)C3)C4)CC21 ; CN1CCC2CN(c3ccc4nc5n(c4c3)[C@@]34CC[C@@H](CN(CCOc6c(cnn6C)-c6cc(cn(C)c6=O)C(=O)N5)C3)C4)CC21]

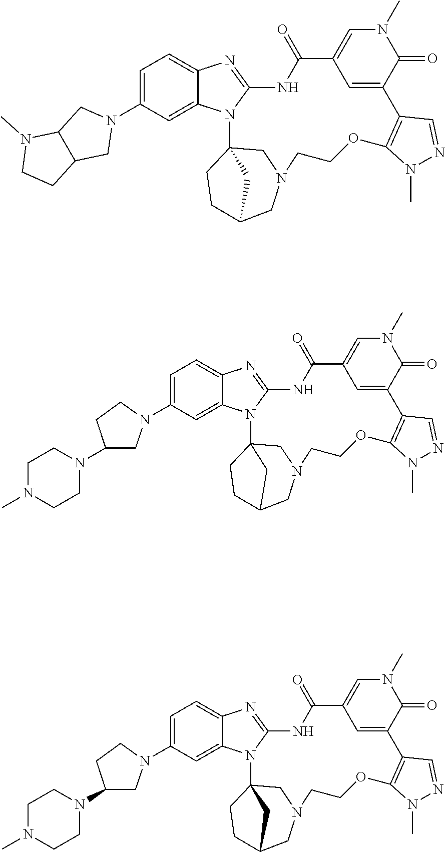 [SMILES: CN1CCC2CN(c3ccc4nc5n(c4c3)[C@]34CC[C@H](CN(CCOc6c(cnn6C)-c6cc(cn(C)c6=O)C(=O)N5)C3)C4)CC21 ; CN1CCN(C2CCN(c3ccc4nc5n(c4c3)C34CCC(CN(CCOc6c(cnn6C)-c6cc(cn(C)c6=O)C(=O)N5)C3)C4)C2)CC1 ; CN1CCN([C@H]2CCN(c3ccc4nc5n(c4c3)[C@@]34CC[C@@H](CN(CCOc6c(cnn6C)-c6cc(cn(C)c6=O)C(=O)N5)C3)C4)C2)CC1]

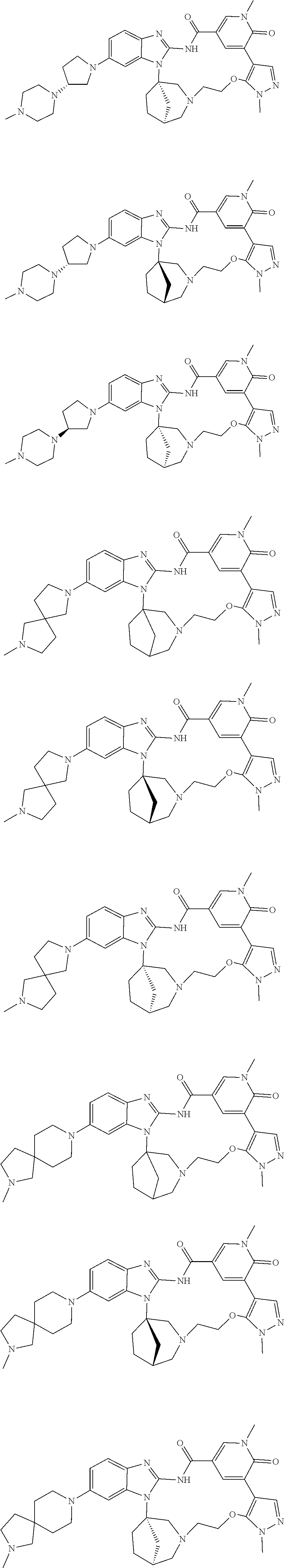

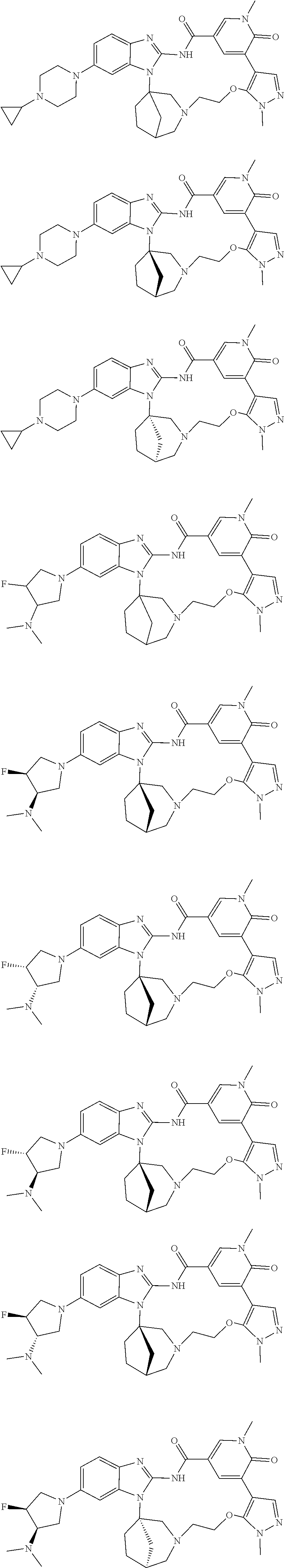

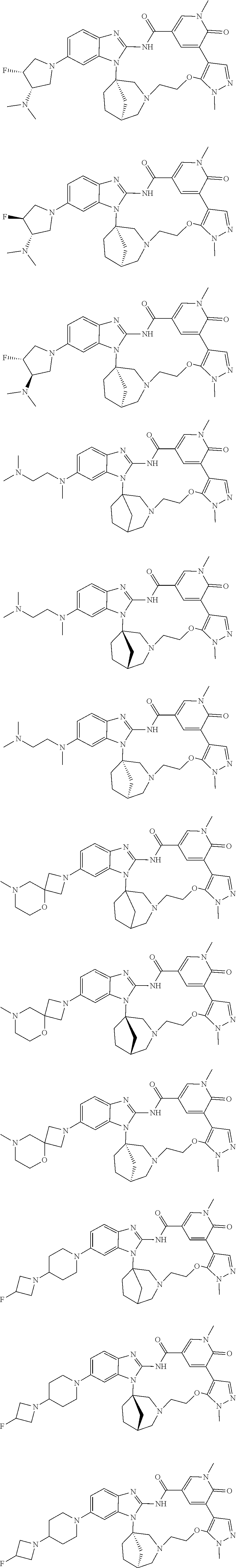

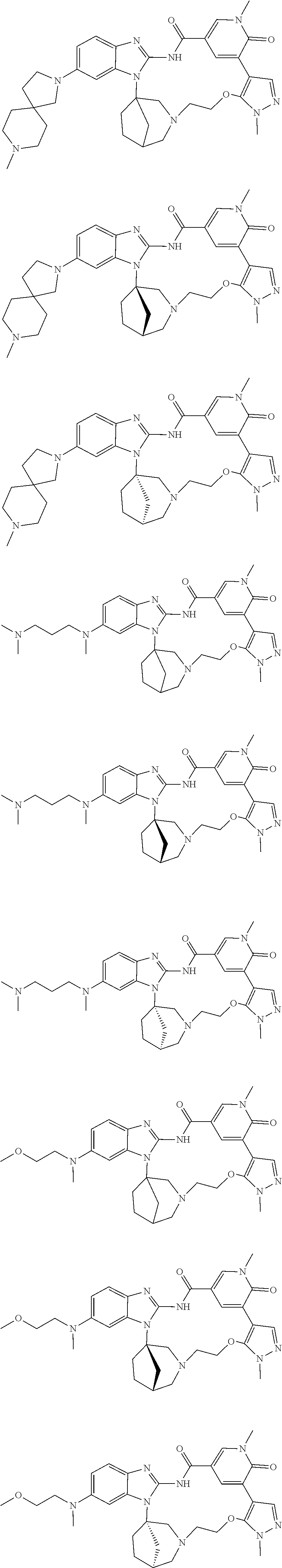

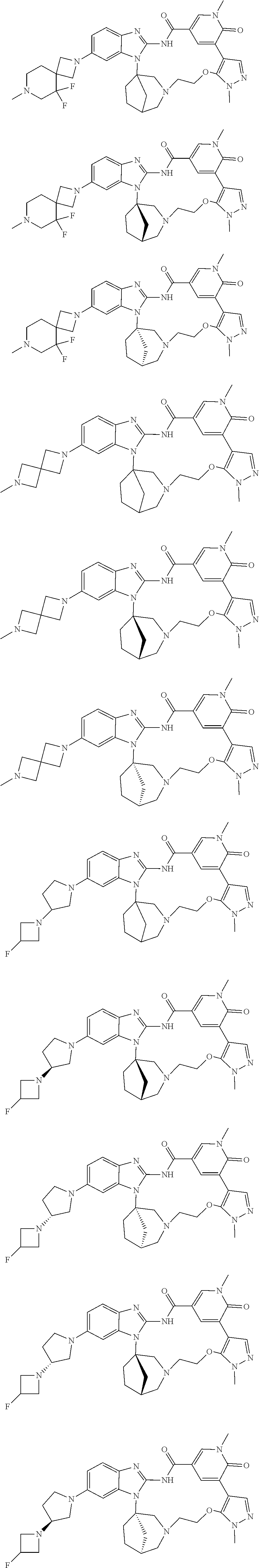

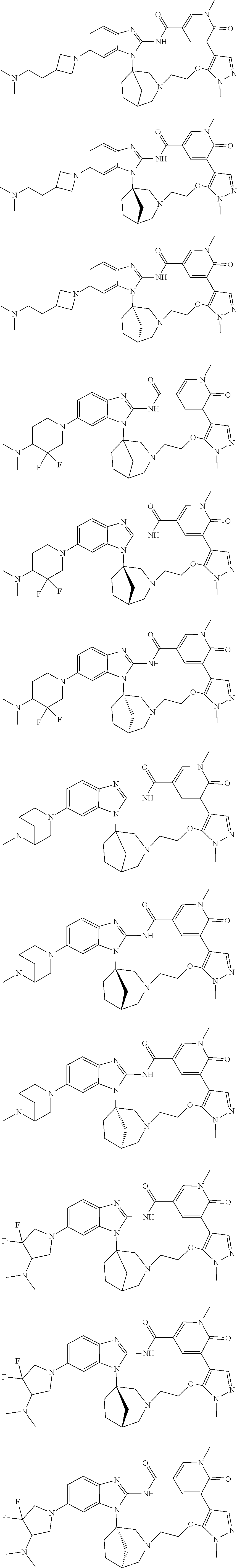

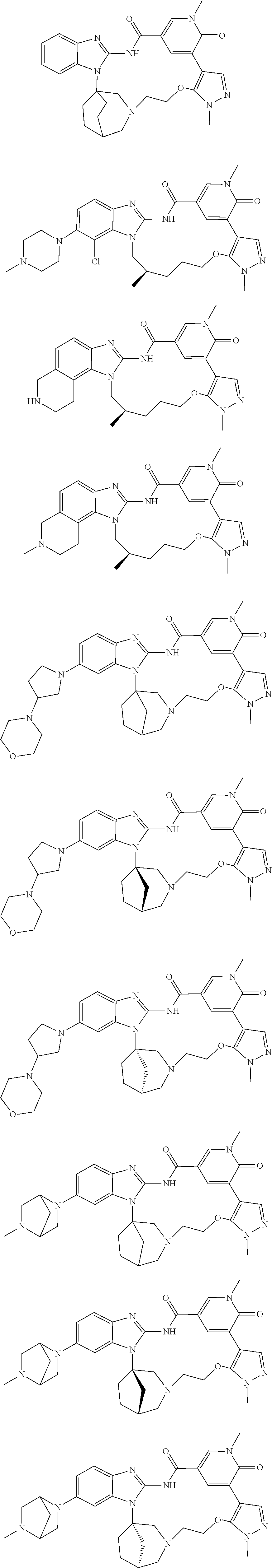

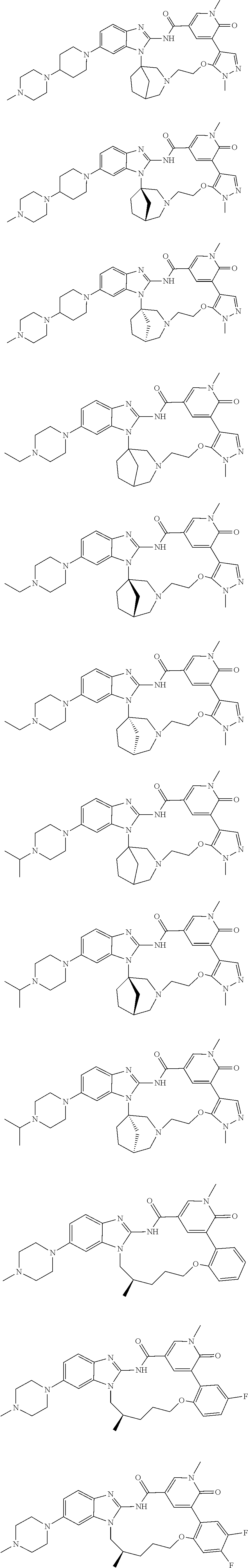

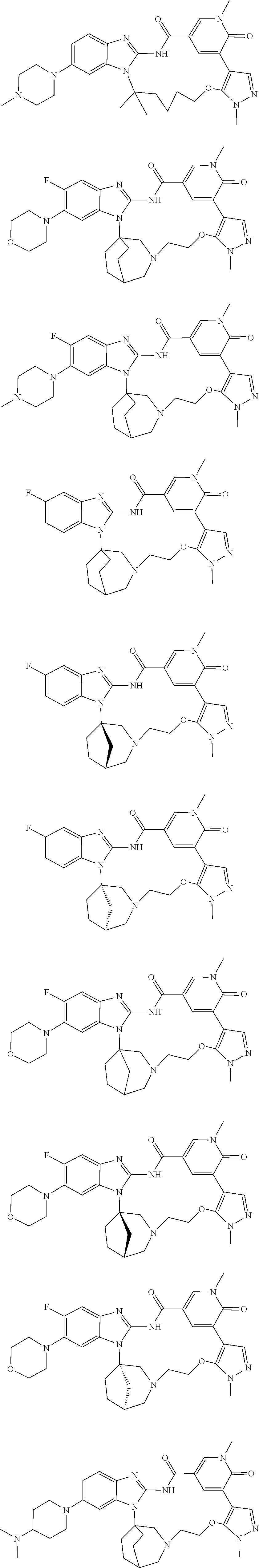

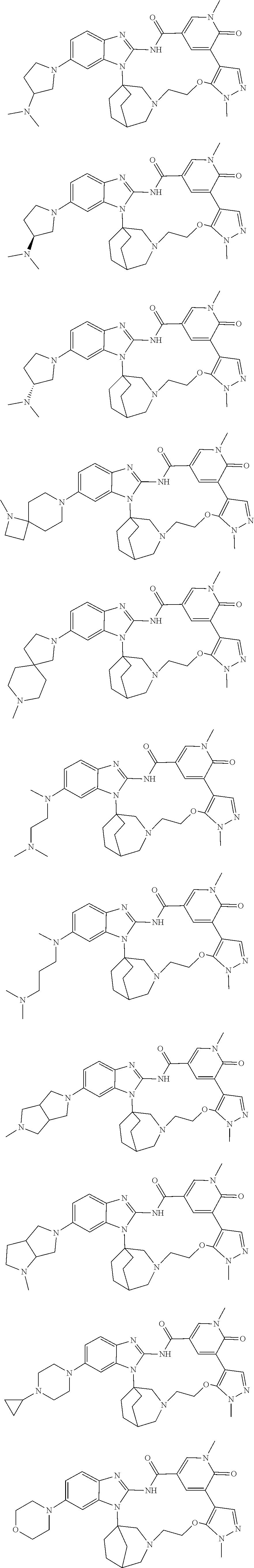

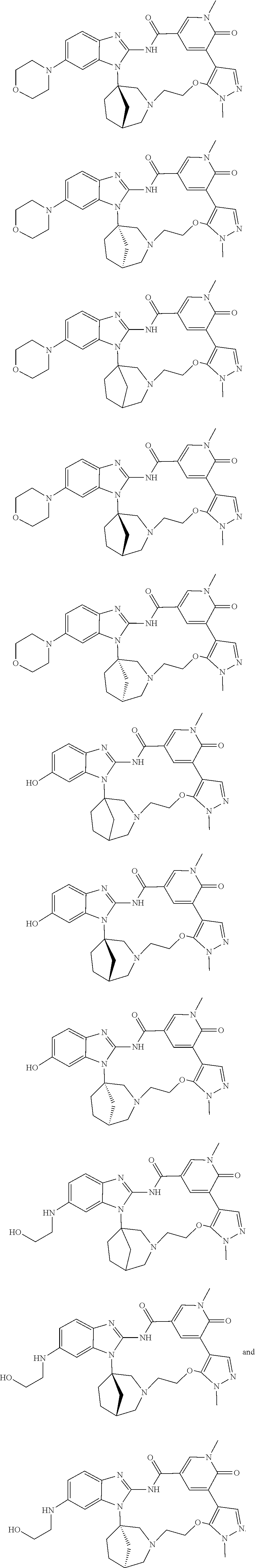

In another aspect, the present disclosure provides a compound of Formula (II):

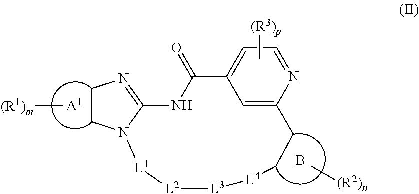

or a pharmaceutically acceptable salt thereof, wherein:
Ring A¹ is 7-12 membered cycloalkyl, 7-12 membered heterocyclyl, 7-12 membered aryl or 7-12 membered heteroaryl;
Ring B is selected from the group consisting of cycloalkyl, heterocyclyl, aryl, and heteroaryl;
L¹ is selected from the group consisting of a bond, alkyl, alkenyl, alkynyl, heteroalkyl, heteroalkenyl and heteroalkynyl, wherein the alkyl, alkenyl, alkynyl, heteroalkyl, heteroalkenyl and heteroalkynyl are optionally substituted with one or more groups independently selected from hydroxyl, halogen, cyano and amino;
L² is selected from the group consisting of a bond, N(R^{A}), alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more R^{B};
wherein R^{A} is selected from a group consisting of hydrogen, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, amino, alkyl and haloalkyl;
wherein each R^{B} is independently selected from a group consisting of hydroxyl, halogen, cyano, amino, alkyl, alkenyl, alkynyl, heteroalkyl, heteroalkenyl, cycloalkyl, heterocyclyl, aryl and heteroaryl;
L³ is selected from the group consisting of a bond, alkyl, alkenyl, alkynyl, heteroalkyl, heteroalkenyl and heteroalkynyl, wherein the alkyl, alkenyl, alkynyl, heteroalkyl, heteroalkenyl and heteroalkynyl are optionally substituted with one or more groups independently selected from hydroxyl, halogen, cyano and amino;
L⁴ is selected from O, S, or N(R^{C});
wherein R^{C} is selected from a group consisting of hydrogen, alkyl, alkenyl, alkynyl, haloalkyl, heteroalkyl, heteroalkenyl, heteroalkynyl, cycloalkyl, heterocyclyl, aryl, and heteroaryl;
each R¹ is independently selected from the group consisting of hydroxy, halogen, cyano, amino, —N(R^{D})₂, alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more R^{E};
wherein each R^{D} is independently selected from a group consisting of hydrogen, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, —N(R^{F})₂ or —OR^{G};
wherein each R^{E} is independently selected from a group consisting of hydrogen, hydroxyl, halogen, cyano, amino, —N(R^{F})₂, -alkyl-N(R^{F})₂, —C(O)OR^{G}, alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, amino and alkyl;
wherein each of R^{F} and R^{G} is independently selected from a group consisting of alkyl, alkenyl, alkynyl, heteroalkyl, heteroalkenyl, heteroalkynyl, cycloalkyl, heterocyclyl, aryl and heteroaryl;
each R² is independently selected from the group consisting of hydrogen, hydroxy, halogen, cyano, amino, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, amino, alkyl and haloalkyl;
each R³ is selected from the group consisting of hydrogen, alkyl, alkenyl, alkynyl, heteroalkyl, heteroalkenyl, heteroalkynyl, cycloalkyl and heterocyclyl, wherein the alkyl, alkenyl, alkynyl, heteroalkyl, heteroalkenyl, heteroalkynyl, cycloalkyl and heterocyclyl are optionally substituted with one or more groups independently selected from hydroxyl, halogen, cyano, amino, alkyl and haloalkyl;
m is an integer from 0 to 5;
n is an integer from 0 to 4; and
p is an integer from 0 to 3.

In another aspect, the present disclosure provides a compound of Formula (III)

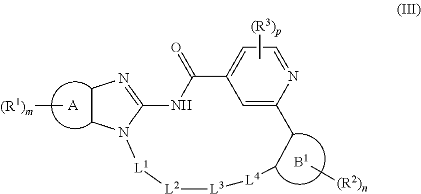

or a pharmaceutically acceptable salt thereof, wherein:
Ring A is selected from the group consisting of cycloalkyl, heterocyclyl, aryl and heteroaryl;
Ring B¹ is 7-12 membered cycloalkyl, 7-12 membered heterocyclyl, 7-12 membered aryl or 7-12 membered heteroaryl;
L¹ is selected from the group consisting of a bond, alkyl, alkenyl, alkynyl, heteroalkyl, heteroalkenyl and heteroalkynyl, wherein the alkyl, alkenyl, alkynyl, heteroalkyl, heteroalkenyl and heteroalkynyl are optionally substituted with one or more groups independently selected from hydroxyl, halogen, cyano and amino;
L² is selected from the group consisting of a bond, N(R^{A}), alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more R^{B};
wherein R^{A} is selected from a group consisting of hydrogen, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, amino, alkyl and haloalkyl;
wherein each R^{B} is independently selected from a group consisting of hydroxyl, halogen, cyano, amino, alkyl, alkenyl, alkynyl, heteroalkyl, heteroalkenyl, cycloalkyl, heterocyclyl, aryl and heteroaryl;
L³ is selected from the group consisting of a bond, alkyl, alkenyl, alkynyl, heteroalkyl, heteroalkenyl and heteroalkynyl, wherein the alkyl, alkenyl, alkynyl, heteroalkyl, heteroalkenyl and heteroalkynyl are optionally substituted with one or more groups independently selected from hydroxyl, halogen, cyano and amino;
L⁴ is selected from O, S, or N(R^{C});
wherein R^{C} is selected from a group consisting of hydrogen, alkyl, alkenyl, alkynyl, haloalkyl, heteroalkyl, heteroalkenyl, heteroalkynyl, cycloalkyl, heterocyclyl, aryl, and heteroaryl;
each R¹ is independently selected from the group consisting of hydroxy, halogen, cyano, amino, —N(R^{D})₂, alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more R^{E};
wherein each R^{D} is independently selected from a group consisting of hydrogen, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, —N(R^{F})₂ or —OR^{G};
wherein each R^{E} is independently selected from a group consisting of hydrogen, hydroxyl, halogen, cyano, amino, —N(R^{F})₂, -alkyl-N(R^{F})₂, —C(O)OR^{G}, alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, amino and alkyl;
wherein each of R^{F} and R^{G} is independently selected from a group consisting of alkyl, alkenyl, alkynyl, heteroalkyl, heteroalkenyl, heteroalkynyl, cycloalkyl, heterocyclyl, aryl and heteroaryl;
each R² is independently selected from the group consisting of hydrogen, hydroxy, halogen, cyano, amino, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, amino, alkyl and haloalkyl;
each R³ is selected from the group consisting of hydrogen, halogen, alkyl, alkenyl, alkynyl, heteroalkyl, heteroalkenyl, heteroalkynyl, cycloalkyl and heterocyclyl, wherein the alkyl, alkenyl, alkynyl, heteroalkyl, heteroalkenyl, heteroalkynyl, cycloalkyl and heterocyclyl are optionally substituted with one or more groups independently selected from hydroxyl, halogen, cyano, amino, alkyl and haloalkyl;
m is an integer from 0 to 5;
n is an integer from 0 to 4; and
p is an integer from 0 to 3.

In another aspect, the present disclosure provides a compound of Formula (IV):

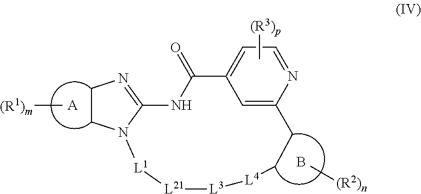

or a pharmaceutically acceptable salt thereof, wherein:
Ring A is selected from the group consisting of cycloalkyl, heterocyclyl, aryl and heteroaryl;
Ring B is selected from the group consisting of cycloalkyl, heterocyclyl, aryl, and heteroaryl;
L¹ is selected from the group consisting of a bond, alkyl, alkenyl, alkynyl, heteroalkyl, heteroalkenyl and heteroalkynyl, wherein the alkyl, alkenyl, alkynyl, heteroalkyl, heteroalkenyl and heteroalkynyl are optionally substituted with one or more groups independently selected from hydroxyl, halogen, cyano and amino;
L²¹ is 7-12 membered cycloalkyl or 7-12 membered heterocyclyl, wherein the cycloalkyl and heterocyclyl are optionally substituted with one or more R^{B};
wherein each R^{B} is independently selected from a group consisting of hydroxyl, halogen, cyano, amino, alkyl, alkenyl, alkynyl, heteroalkyl, heteroalkenyl, cycloalkyl, heterocyclyl, aryl and heteroaryl;
L³ is selected from the group consisting of a bond, alkyl, alkenyl, alkynyl, heteroalkyl, heteroalkenyl and heteroalkynyl, wherein the alkyl, alkenyl, alkynyl, heteroalkyl, heteroalkenyl and heteroalkynyl are optionally substituted with one or more groups independently selected from hydroxyl, halogen, cyano and amino;
L⁴ is selected from O, S, or N(R^{C});
wherein R^{C} is selected from a group consisting of hydrogen, alkyl, alkenyl, alkynyl, haloalkyl, heteroalkyl, heteroalkenyl, heteroalkynyl, cycloalkyl, heterocyclyl, aryl, and heteroaryl;
each R¹ is independently selected from the group consisting of hydroxy, halogen, cyano, amino, —N(R^{D})₂, alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more R^{E};
wherein each R^{D} is independently selected from a group consisting of hydrogen, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, —N(R^{F})₂ or —OR^{G};
wherein each R^{E} is independently selected from a group consisting of hydrogen, hydroxyl, halogen, cyano, amino, —N(R^{F})₂, -alkyl-N(R^{F})₂, —C(O)OR^{G}, alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, amino and alkyl;
wherein each of R^{F} and R^{G} is independently selected from a group consisting of alkyl, alkenyl, alkynyl, heteroalkyl, heteroalkenyl, heteroalkynyl, cycloalkyl, heterocyclyl, aryl and heteroaryl;
each R² is independently selected from the group consisting of hydrogen, hydroxy, halogen, cyano, amino, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, amino, alkyl and haloalkyl;
R³ is selected from the group consisting of hydrogen, alkyl, alkenyl, alkynyl, heteroalkyl, heteroalkenyl, heteroalkynyl, cycloalkyl and heterocyclyl, wherein the alkyl, alkenyl, alkynyl, heteroalkyl, heteroalkenyl, heteroalkynyl, cycloalkyl and heterocyclyl are optionally substituted with one or more groups independently selected from hydroxyl, halogen, cyano, amino, alkyl and haloalkyl;
m is an integer from 0 to 5;
n is an integer from 0 to 4; and
p is an integer from 0 to 3.

In another aspect, the present disclosure provides a compound or a pharmaceutically acceptable salt thereof, wherein the compound is selected from a group consisting of:

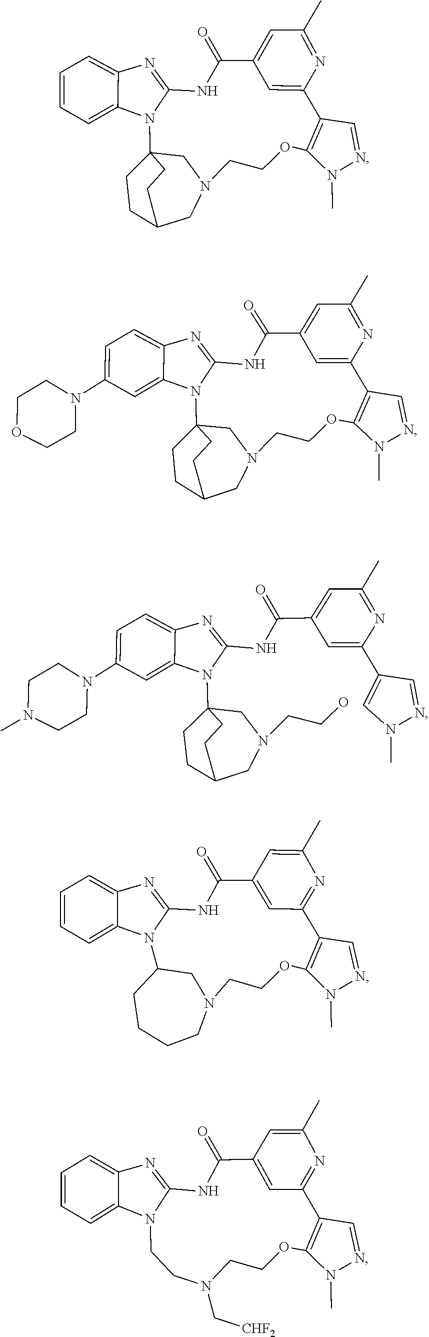

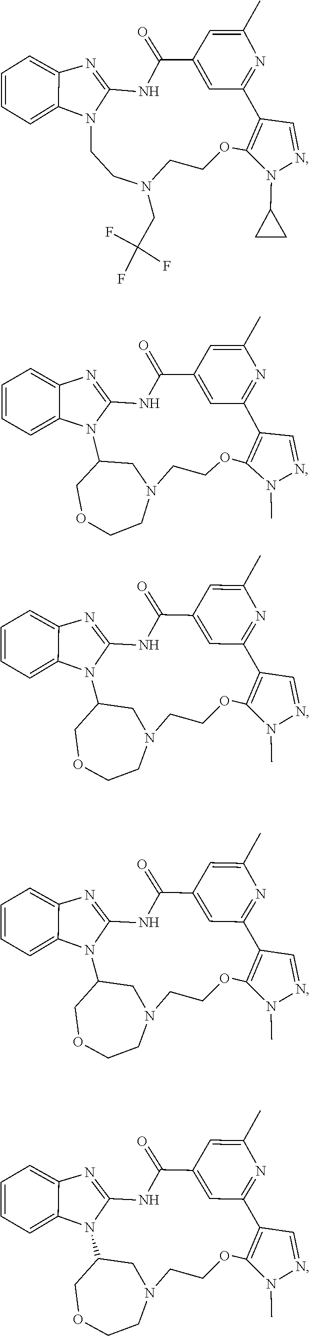

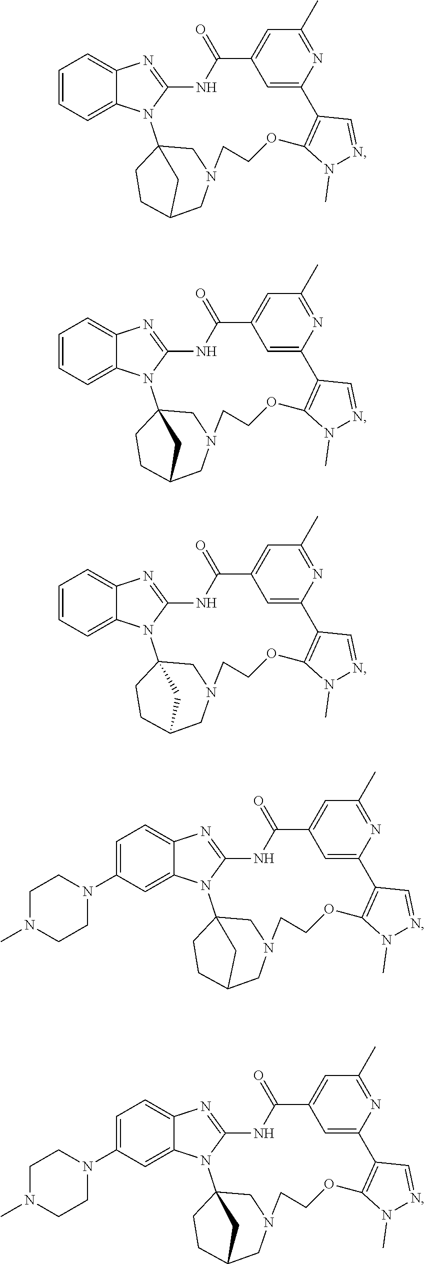 [SMILES: Cc1cc2cc(n1)-c1cnn(C)c1OCCN1CC3CCC(C3)(C1)n1c(nc3ccccc31)NC2=O ; Cc1cc2cc(n1)-c1cnn(C)c1OCCN1C[C@@H]3CC[C@@](C3)(C1)n1c(nc3ccccc31)NC2=O ; Cc1cc2cc(n1)-c1cnn(C)c1OCCN1C[C@H]3CC[C@](C3)(C1)n1c(nc3ccccc31)NC2=O ; Cc1cc2cc(n1)-c1cnn(C)c1OCCN1CC3CCC(C3)(C1)n1c(nc3ccc(N4CCN(C)CC4)cc31)NC2=O ; Cc1cc2cc(n1)-c1cnn(C)c1OCCN1C[C@@H]3CC[C@@](C3)(C1)n1c(nc3ccc(N4CCN(C)CC4)cc31)NC2=O]

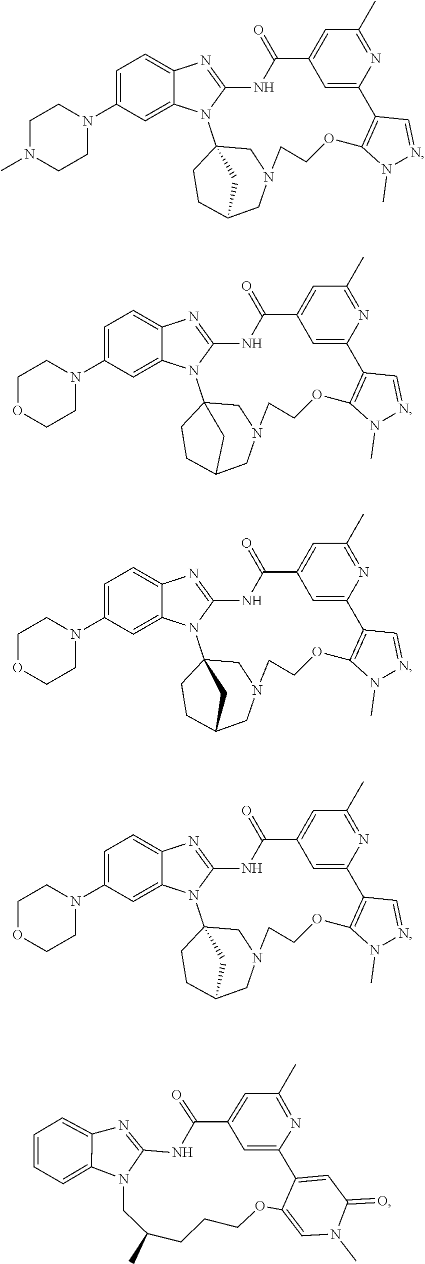 [SMILES: Cc1cc2cc(n1)-c1cnn(C)c1OCCN1C[C@H]3CC[C@](C3)(C1)n1c(nc3ccc(N4CCN(C)CC4)cc31)NC2=O ; Cc1cc2cc(n1)-c1cnn(C)c1OCCN1CC3CCC(C3)(C1)n1c(nc3ccc(N4CCOCC4)cc31)NC2=O ; Cc1cc2cc(n1)-c1cnn(C)c1OCCN1C[C@@H]3CC[C@@](C3)(C1)n1c(nc3ccc(N4CCOCC4)cc31)NC2=O ; Cc1cc2cc(n1)-c1cnn(C)c1OCCN1C[C@H]3CC[C@](C3)(C1)n1c(nc3ccc(N4CCOCC4)cc31)NC2=O ; Cc1cc2cc(n1)-c1cc(=O)n(C)cc1OCCC[C@@H](C)Cn1c(nc3ccccc31)NC2=O]

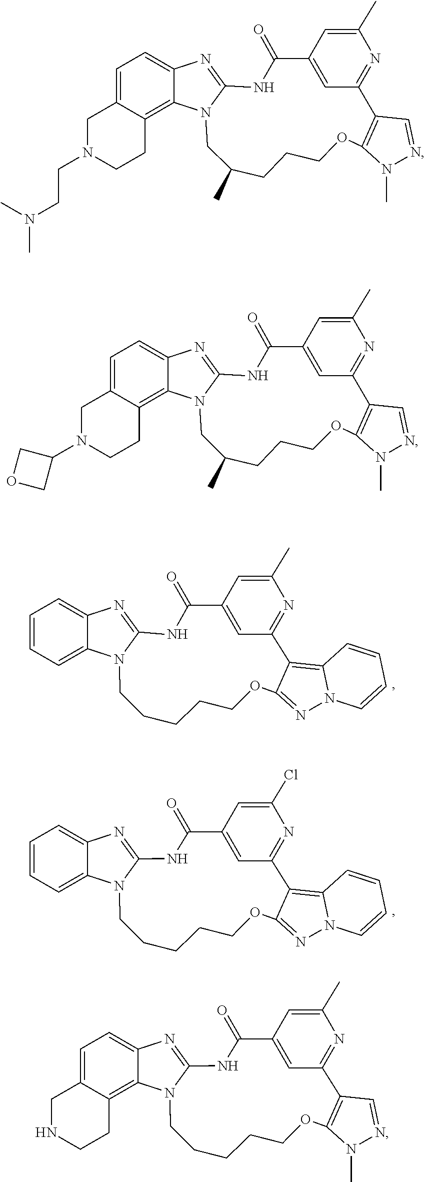

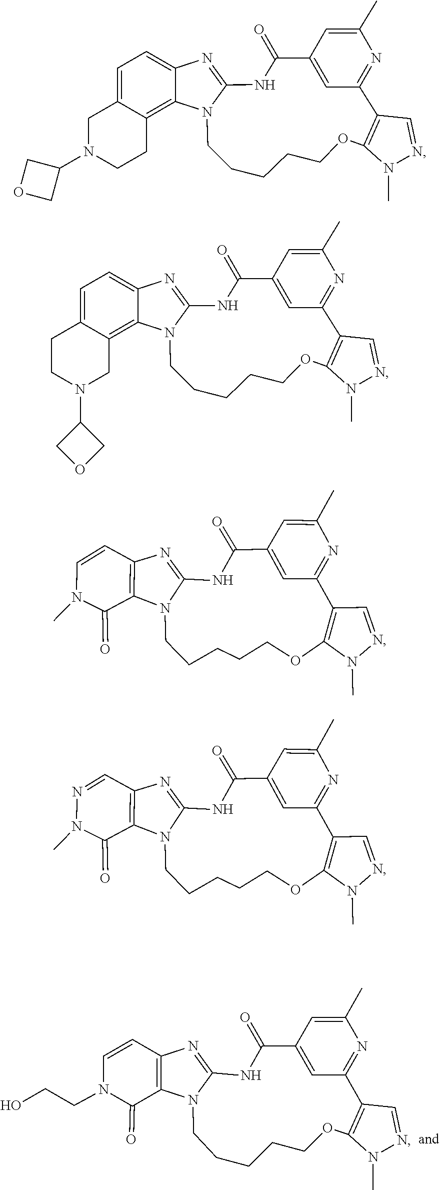

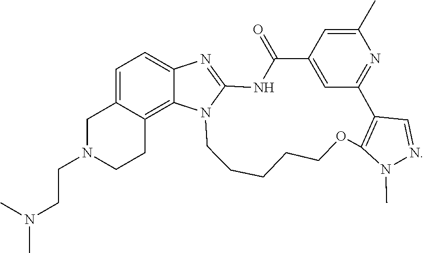

In another aspect, the present disclosure provides a pharmaceutical composition comprising the compound of the present disclosure or a pharmaceutically acceptable salt thereof, and a pharmaceutically acceptable carrier.

In a further aspect, the present disclosure provides a method for inhibiting EGFR activity in a subject in need thereof, comprising administering an effective amount of a compound of the present disclosure or a pharmaceutically acceptable salt thereof or the pharmaceutical composition of the present disclosure to the subject.

In a further aspect, the present disclosure provides a method for treating an EGFR related disorder comprising administering an effective amount of a compound of the present disclosure or a pharmaceutically acceptable salt thereof or the pharmaceutical composition of the present disclosure to a subject in need thereof.

In another aspect, the present disclosure provides use of the compound of the present disclosure or a pharmaceutically acceptable salt thereof or the pharmaceutical composition of the present disclosure in the manufacture of a medicament for treating EGFR-related disorders.

In another aspect, the present disclosure provides a compound of present disclosure or a pharmaceutically acceptable salt thereof or the pharmaceutical composition of the present disclosure, for use in the treatment of EGFR-related disorder.

### DETAILED DESCRIPTION OF THE DISCLOSURE

Reference will now be made in detail to certain embodiments of the present disclosure, examples of which are illustrated in the accompanying structures and formulas. While the present disclosure will be described in conjunction with the enumerated embodiments, it will be understood that they are not intended to limit the present disclosure to those embodiments. On the contrary, the present disclosure is intended to cover all alternatives, modifications, and equivalents, which may be included within the scope of the present disclosure as defined by the claims. One skilled in the art will recognize many methods and materials similar or equivalent to those described herein, which could be used in the practice of the present disclosure. The present disclosure is in no way limited to the methods and materials described. In the event that one or more of the incorporated references and similar materials differs from or contradicts this application, including but not limited to defined terms, term usage, described techniques, or the like, the present disclosure controls. All references, patents, patent applications cited in the present disclosure are hereby incorporated by reference in their entireties.

It is appreciated that certain features of the present disclosure, which are, for clarity, described in the context of separate embodiments, can also be provided in combination in a single embodiment. Conversely, various features of the present disclosure, which are, for brevity, described in the context of a single embodiment, can also be provided separately or in any suitable sub-combination. It must be noted that, as used in the specification and the appended claims, the singular forms “a,” “an,” and “the” include plural forms of the same unless the context clearly dictates otherwise. Thus, for example, reference to “a compound” includes a plurality of compounds.

### DEFINITION

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; LaEGFR, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

At various places in the present disclosure, linking substituents are described. Where the structure clearly requires a linking group, the Markush variables listed for that group are understood to be linking groups. For example, if the structure requires a linking group and the Markush group definition for that variable lists “alkyl”, then it is understood that the “alkyl” represents a linking alkylene group.

When any variable (e.g., Rⁱ) occurs more than one time in any constituent or formula for a compound, its definition at each occurrence is independent of its definition at every other occurrence. Thus, for example, if a group is shown to be substituted with 0-2 Rⁱ moieties, then the group may optionally be substituted with up to two R moieties and R at each occurrence is selected independently from the definition of Rⁱ. Also, combinations of substituents and/or variables are permissible, but only if such combinations result in stable compounds.

As used herein, a dash “-” at the front or end of a chemical group is used, a matter of convenience, to indicate a point of attachment for a substituent. For example, —OH is attached through the oxygen atom; chemical groups may be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named. As used herein, a solid line coming out of the center of a ring indicates that the point of attachment for a substituent on the ring can be at any ring atom. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

Recitation of ranges of values are merely intended to serve as a shorthand method of referring individually to each separate value falling within the range, unless otherwise indicated herein, and each separate value is incorporated into the specification as if it were individually recited herein. A range used herein, unless otherwise specified, includes the two limits of the range. For example, the expressions “n is an integer between 1 and 6” and “n being an integer of 1 to 6” both mean “n being 1, 2, 3, 4, 5, or 6”.

As used herein, the term “compounds provided herein”, or “compounds disclosed herein” or “compounds of the present disclosure” refers to the compounds of Formula (T), (II), (III) and (IV) as well as the specific compounds disclosed herein.

As used herein, the term “Cᵢ₋ⱼ” indicates a range of the carbon atoms numbers, wherein i and j are integers and the range of the carbon atoms numbers includes the endpoints (i.e. i and j) and each integer point in between, and wherein j is greater than i. For examples, C₁₋₆ indicates a range of one to six carbon atoms, including one carbon atom, two carbon atoms, three carbon atoms, four carbon atoms, five carbon atoms and six carbon atoms. In some embodiments, the term “C₁₋₁₂” indicates 1 to 12, particularly 1 to 10, particularly 1 to 8, particularly 1 to 6, particularly 1 to 5, particularly 1 to 4, particularly 1 to 3 or particularly 1 to 2 carbon atoms.

As used herein, the term “alkyl”, whether as part of another term or used independently, refers to a saturated linear or branched-chain hydrocarbon radical, which may be optionally substituted independently with one or more substituents described below. The term “Cᵢ₋ⱼ alkyl” refers to an alkyl having i to j carbon atoms. In some embodiments, alkyl groups contain 1 to 10 carbon atoms. In some embodiments, alkyl groups contain 1 to 9 carbon atoms. In some embodiments, alkyl groups contain 1 to 8 carbon atoms, 1 to 7 carbon atoms, 1 to 6 carbon atoms, 1 to 5 carbon atoms, 1 to 4 carbon atoms, 1 to 3 carbon atoms, or 1 to 2 carbon atoms. Examples of “C₁₋₁₀ alkyl” include, but are not limited to, methyl, ethyl, propyl, butyl, pentyl, hexyl, heptyl, octyl, nonyl, and decyl. Examples of “C₁₋₆ alkyl” are methyl, ethyl, propyl, isopropyl, n-butyl, i-butyl, s-butyl, t-butyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, and the like.

As used herein, the term “alkenyl”, whether as part of another term or used independently, refers to linear or branched-chain hydrocarbon radical having at least one carbon-carbon double bond, which may be optionally substituted independently with one or more substituents described herein, and includes radicals having “cis” and “trans” orientations, or alternatively, “E” and “Z” orientations. In some embodiments, alkenyl groups contain 2 to 12 carbon atoms. In some embodiments, alkenyl groups contain 2 to 11 carbon atoms. In some embodiments, alkenyl groups contain 2 to 11 carbon atoms, 2 to 10 carbon atoms, 2 to 9 carbon atoms, 2 to 8 carbon atoms, 2 to 7 carbon atoms, 2 to 6 carbon atoms, 2 to 5 carbon atoms, 2 to 4 carbon atoms, 2 to 3 carbon atoms, and in some embodiments, alkenyl groups contain 2 carbon atoms. Examples of alkenyl group include, but are not limited to, ethylenyl (or vinyl), propenyl (allyl), butenyl, pentenyl, 1-methyl-2 buten-1-yl, 5-hexenyl, and the like.

As used herein, the term “alkynyl”, whether as part of another term or used independently, refers to a linear or branched hydrocarbon radical having at least one carbon-carbon triple bond, which may be optionally substituted independently with one or more substituents described herein. In some embodiments, alkenyl groups contain 2 to 12 carbon atoms. In some embodiments, alkynyl groups contain 2 to 11 carbon atoms. In some embodiments, alkynyl groups contain 2 to 11 carbon atoms, 2 to 10 carbon atoms, 2 to 9 carbon atoms, 2 to 8 carbon atoms, 2 to 7 carbon atoms, 2 to 6 carbon atoms, 2 to 5 carbon atoms, 2 to 4 carbon atoms, 2 to 3 carbon atoms, and in some embodiments, alkynyl groups contain 2 carbon atoms. Examples of alkynyl group include, but are not limited to, ethynyl, 1-propynyl, 2-propynyl, and the like.

As used herein, the term “amino” refers to —NH₂ group. Amino groups may also be substituted with one or more groups such as alkyl, alkenyl, alkynyl, aryl, carbonyl or other amino groups.

As used herein, the term “aryl”, whether as part of another term or used independently, refers to monocyclic and polycyclic ring systems having a total of 5 to 20 ring members, wherein at least one ring in the system is aromatic and wherein each ring in the system contains 3 to 12 ring members. Examples of “aryl” include, but are not limited to, phenyl, biphenyl, naphthyl, anthracyl and the like, which may bear one or more substituents. Also included within the scope of the term “aryl”, as it is used herein, is a group in which an aromatic ring is fused to one or more additional rings. In the case of polycyclic ring system, only one of the rings needs to be aromatic (e.g., 2,3-dihydroindole), although all of the rings may be aromatic (e.g., quinoline). The second ring can also be fused or bridged. Examples of polycyclic aryl include, but are not limited to, benzofuranyl, indanyl, phthalimidyl, naphthimidyl, phenanthridinyl, or tetrahydronaphthyl, and the like. Aryl groups can be substituted at one or more ring positions with substituents as described above.

As used herein, the term “cyano” refers to —CN.

As used herein, the term “cycloalkyl”, whether as part of another term or used independently, refer to a monovalent non-aromatic, saturated or partially unsaturated monocyclic and polycyclic ring system, in which all the ring atoms are carbon and which contains at least three ring forming carbon atoms. In some embodiments, the cycloalkyl may contain 3 to 12 ring forming carbon atoms, 3 to 10 ring forming carbon atoms, 3 to 9 ring forming carbon atoms, 3 to 8 ring forming carbon atoms, 3 to 7 ring forming carbon atoms, 3 to 6 ring forming carbon atoms, 3 to 5 ring forming carbon atoms, 4 to 12 ring forming carbon atoms, 4 to 10 ring forming carbon atoms, 4 to 9 ring forming carbon atoms, 4 to 8 ring forming carbon atoms, 4 to 7 ring forming carbon atoms, 4 to 6 ring forming carbon atoms, 4 to 5 ring forming carbon atoms. Cycloalkyl groups may be saturated or partially unsaturated. Cycloalkyl groups may be substituted. In some embodiments, the cycloalkyl group may be a saturated cyclic alkyl group. In some embodiments, the cycloalkyl group may be a partially unsaturated cyclic alkyl group that contains at least one double bond or triple bond in its ring system. In some embodiments, the cycloalkyl group may be monocyclic or polycyclic. In the case of polycyclic ring system, the fused, spiro and bridged ring systems are included within the scope of this definition. Examples of monocyclic cycloalkyl group include, but are not limited to, cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl and cyclododecyl. Examples of polycyclic cycloalkyl group include, but are not limited to, adamantyl, norbornyl, fluorenyl, spiro-pentadienyl, spiro[3.6]-decanyl, bicyclo[1,1,1]pentenyl, bicyclo[2,2,1]heptenyl, and the like.

As used herein, the term “halogen” refers to an atom selected from fluorine (or fluoro), chlorine (or chloro), bromine (or bromo) and iodine (or iodo).

As used herein, the term “heteroatom” refers to nitrogen, oxygen, sulfur, phosphorus, and includes any oxidized form of nitrogen, sulfur or phosphorus, and any quaternized form of a basic nitrogen (including N-oxides).

As used herein, the term “heteroalkyl” refers to an alkyl, at least one of the carbon atoms of which is replaced with a heteroatom selected from N, O, or S. The heteroalkyl may be a carbon radical or heteroatom radical (i.e., the heteroatom may appear in the middle or at the end of the radical), and may be optionally substituted independently with one or more substituents described herein. The term “heteroalkyl” encompasses alkoxy and heteroalkoxy radicals.

As used herein, the term “heteroalkenyl” refers to an alkenyl, at least one of the carbon atoms of which is replaced with a heteroatom selected from N, O, or S. The heteroalkenyl may be a carbon radical or heteroatom radical (i.e., the heteroatom may appear in the middle or at the end of the radical), and may be optionally substituted independently with one or more substituents described herein.

As used herein, the term “heteroalkynyl” refers to an alkynyl, at least one of the carbon atoms of which is replaced with a heteroatom selected from N, O, or S. The heteroalkynyl may be a carbon radical or heteroatom radical (i.e., the heteroatom may appear in the middle or at the end of the radical), and may be optionally substituted independently with one or more substituents described herein.

As used herein, the term “heteroaryl”, whether as part of another term or used independently, refers to an aryl group having, in addition to carbon atoms, one or more heteroatoms. The heteroaryl group can be monocyclic. Examples of monocyclic heteroaryl include, but are not limited to, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, benzofuranyl and pteridinyl. The heteroaryl group also includes polycyclic groups in which a heteroaromatic ring is fused to one or more aryl, cycloalkyl, or heterocyclyl rings, where the radical or point of attachment is on the heteroaromatic ring. Examples of polycyclic heteroaryl include, but are not limited to, indolyl, isoindolyl, benzothienyl, benzofuranyl, benzo[1,3]dioxolyl, dibenzofuranyl, indazolyl, benzimidazolyl, benzthiazolyl, quinolyl, isoquinolyl, dihydroquinolinyl, dihydroisoquinolinyl, tetrahydroquinolinyl, tetrahydroisoquinolinyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and the like.

As used herein, the term “heterocyclyl” refers to a saturated or partially unsaturated carbocyclyl group in which one or more ring atoms are heteroatoms independently selected from oxygen, sulfur, nitrogen, phosphorus, and the like, the remaining ring atoms being carbon, wherein one or more ring atoms may be optionally substituted independently with one or more substituents. In some embodiments, the heterocyclyl is a saturated heterocyclyl. In some embodiments, the heterocyclyl is a partially unsaturated heterocyclyl having one or more double bonds in its ring system. In some embodiments, the heterocyclyl may contains any oxidized form of carbon, nitrogen, sulfur or phoshporus, and any quaternized form of a basic nitrogen. “Heterocyclyl” also includes radicals wherein the heterocyclyl radicals are fused with a saturated, partially unsaturated, or fully unsaturated (i.e., aromatic) carbocyclic or heterocyclic ring. The heterocyclyl radical may be carbon linked or nitrogen linked where such is possible. In some embodiments, the heterocycle is carbon linked. In some embodiments, the heterocycle is nitrogen linked. For example, a group derived from pyrrole may be pyrrol-1-yl (nitrogen linked) or pyrrol-3-yl (carbon linked). Further, a group derived from imidazole may be imidazol-1-yl (nitrogen linked) or imidazol-3-yl (carbon linked).

In some embodiments, the term “3- to 12-membered heterocyclyl” refers to a 3- to 12-membered saturated or partially unsaturated monocyclic or polycyclic heterocyclic ring system having 1 to 3 heteroatoms independently selected from nitrogen, oxygen, sulfur or phosphorus. In the case of polycyclic ring system, the fused, spiro and bridged ring systems are also included within the scope of this definition. Examples of monocyclic heterocyclyl include, but are not limited to oxetanyl, 1,1-dioxothietanylpyrrolidyl, tetrahydrofuryl, tetrahydrothienyl, pyrrolyl, furanyl, thienyl, pyrazolyl, imidazolyl, triazolyl, oxazolyl, thiazolyl, piperidyl, piperazinyl, piperidinyl, morpholinyl, pyridinyl, pyrazinyl, pyrimidinyl, pyridazinyl, triazinyl, pyridonyl, pyrimidonyl, pyrazinonyl, pyrimidonyl, pyridazonyl, pyrrolidinyl, triazinonyl, and the like. Examples of fused heterocyclyl include, but are not limited to, quinolinyl, isoquinolinyl, tetrahydroquinolinyl, tetrahydroisoquinolinyl, quinoxalinyl, quinolizinyl, quinazolinyl, azaindolizinyl, pteridinyl, chromenyl, isochromenyl, indolyl, isoindolyl, indolizinyl, indazolyl, purinyl, benzofuranyl, isobenzofuranyl, benzimidazolyl, benzothienyl, benzothiazolyl, carbazolyl, phenazinyl, phenothiazinyl, phenanthridinyl, hexahydro-1H-pyrrolizinyl, imidazo[1,2-a]pyridinyl, [1,2,4]triazolo[4,3-a]pyridinyl, [1,2,3]triazolo[4,3-a]pyridinyl, octahydropyrrolo[3,4-b]pyrrolyl, octahydropyrrolo[3,4-c]pyrrolyl groups, and the like. Examples of spiro heterocyclyl include, but are not limited to, spiropyranyl, spirooxazinyl, 2,6-diazaspiro[3.3]heptanyl, 2,5-diazaspiro[3.4]octanyl, 2,6-diazaspiro[3.4]octanyl, 2,7-diazaspiro[3.5]nonanyl, 5-oxa-2,8-diazaspiro[3.5]nonanyl, 2,7-diazaspiro[4.4]nonanyl, 1,7-diazaspiro[3.5]nonanyl, 2,8-diazaspiro[4.5]decanyl, 2,8-diazaspiro[4.5]decanyl, and the like. Examples of bridged heterocyclyl include, but are not limited to, morphanyl, hexamethylenetetraminyl, 3-aza-bicyclo[3.1.0]hexane, 3,6-diazabicyclo[3.1.1]heptane, 2,5-diazabicyclo[2.2.1]heptane, 8-aza-bicyclo[3.2.1]octane, 3-azabicyclo[3.2.1]octane, 1-aza-bicyclo[2.2.2]octane, 1,4-diazabicyclo[2.2.2]octane, 3-azabicyclo[3.2.2]nonane, and the like.

As used herein, the term “hydroxyl” or “hydroxy” refers to —OH.

As used herein, the term “partially unsaturated” refers to a radical that includes at least one double or triple bond. The term “partially unsaturated” is intended to encompass rings having multiple sites of unsaturation, but is not intended to include aromatic (i.e., fully unsaturated) moieties.

As used herein, the term “optional” or “optionally” means that the subsequently described event or circumstance may or may not occur, and that the description includes instances where the said event or circumstance occurs and instances in which it does not. As used herein, the term “substituted”, whether preceded by the term “optionally” or not, means that one or more hydrogens of the designated moiety are replaced with a suitable substituent. It will be understood that “substitution” or “substituted with” includes the implicit proviso that such substitution is in accordance with permitted valence of the substituted atom and that the substitution results in a stable or chemically feasible compound, e.g., which does not spontaneously undergo transformation such as by rearrangement, cyclization, elimination, etc. Unless otherwise indicated, an “optionally substituted” group may have a suitable substituent at each substitutable position of the group, and when more than one position in any given structure may be substituted with more than one substituent selected from a specified group, the substituent may be either the same or different at every position. The substituents may include, but not limited to, alkyl, alkenyl, alkynyl, alkoxy, acyl, amino, amido, amidino, aryl, azido, carbamoyl, carboxyl, carboxyl ester, cyano, guanidino, halo, haloalkyl, heteroalkyl, heteroaryl, heterocyclyl, hydroxy, hydrazino, imino, oxo, nitro, alkylsulfinyl, sulfonic acid, alkylsulfonyl, thiocyanate, thiol, thione, or combinations thereof. It will be understood by those skilled in the art that substituents can themselves be substituted, if appropriate. Unless specifically stated as “unsubstituted”, references to chemical moieties herein are understood to include substituted variants. For example, reference to an “aryl” group or moiety implicitly includes both substituted and unsubstituted variants.

As used herein, the term “substituted”, whether preceded by the term “optionally” or not, means that one or more hydrogens of the designated moiety are replaced with a suitable substituent. It will be understood that “substitution” or “substituted with” includes the implicit proviso that such substitution is in accordance with permitted valence of the substituted atom and that the substitution results in a stable or chemically feasible compound, e.g., which does not spontaneously undergo transformation such as by rearrangement, cyclization, elimination, etc. Unless otherwise indicated, an “optionally substituted” group may have a suitable substituent at each substitutable position of the group, and when more than one position in any given structure may be substituted with more than one substituent selected from a specified group, the substituent may be either the same or different at every position. It will be understood by those skilled in the art that substituents can themselves be substituted, if appropriate. Unless specifically stated as “unsubstituted”, references to chemical moieties herein are understood to include substituted variants. For example, reference to an “aryl” group or moiety implicitly includes both substituted and unsubstituted variants.

The symbols “R” and “S” represent the configuration of substituents around a chiral carbon atom(s). The isomeric descriptors “R” and “S” are used as described herein for indicating atom configuration(s) relative to a core molecule and are intended to be used as defined in the literature (IUPAC Recommendations 1996, Pure and Applied Chemistry, 68:2193-2222 (1996)).

### Compound

In one aspect, the present disclosure provides compounds of Formula (I):

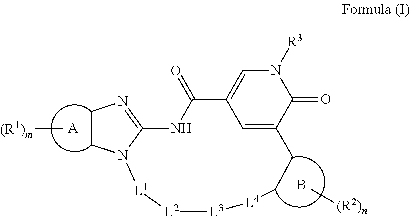

or a pharmaceutically acceptable salt thereof,
wherein,
Ring A is selected from the group consisting of cycloalkyl, heterocyclyl, aryl and heteroaryl;
Ring B is selected from the group consisting of cycloalkyl, heterocyclyl, aryl, and heteroaryl;
L¹ is selected from the group consisting of a bond, alkyl, alkenyl, alkynyl, heteroalkyl, heteroalkenyl and heteroalkynyl, wherein the alkyl, alkenyl, alkynyl, heteroalkyl, heteroalkenyl and heteroalkynyl are optionally substituted with one or more groups independently selected from hydroxyl, halogen, cyano and amino;
L² is selected from the group consisting of a bond, N(R^{A}), alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more R^{B};
wherein R^{A} is selected from a group consisting of hydrogen, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, amino, alkyl and haloalkyl;
wherein each R^{B} is independently selected from a group consisting of hydroxyl, halogen, cyano, amino, alkyl, alkenyl, alkynyl, heteroalkyl, heteroalkenyl, cycloalkyl, heterocyclyl, aryl and heteroaryl;
L³ is selected from the group consisting of a bond, alkyl, alkenyl, alkynyl, heteroalkyl, heteroalkenyl and heteroalkynyl, wherein the alkyl, alkenyl, alkynyl, heteroalkyl, heteroalkenyl and heteroalkynyl are optionally substituted with one or more groups independently selected from hydroxyl, halogen, cyano and amino;
L⁴ is selected from O, S, or N(R^{C});
wherein R^{C} is selected from a group consisting of hydrogen, alkyl, alkenyl, alkynyl, haloalkyl, heteroalkyl, heteroalkenyl, heteroalkynyl, cycloalkyl, heterocyclyl, aryl, and heteroaryl;
each R¹ is independently selected from the group consisting of hydroxy, halogen, cyano, amino, —N(R^{D})₂, alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more R^{E};
wherein each R^{D} is independently selected from a group consisting of hydrogen, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, —N(R^{F})₂ or —OR^{G};
wherein each R^{E} is independently selected from a group consisting of hydrogen, hydroxyl, halogen, cyano, amino, —N(R^{F})₂, -alkyl-N(R^{F})₂, —C(O)OR^{G}, alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, amino and alkyl;
wherein each of R^{F} and R^{G} is independently selected from a group consisting of alkyl, alkenyl, alkynyl, heteroalkyl, heteroalkenyl, heteroalkynyl, cycloalkyl, heterocyclyl, aryl and heteroaryl;
each R² is independently selected from the group consisting of hydrogen, hydroxy, halogen, cyano, amino, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, amino, alkyl and haloalkyl;
R³ is selected from the group consisting of hydrogen, alkyl, alkenyl, alkynyl, heteroalkyl, heteroalkenyl, heteroalkynyl, cycloalkyl and heterocyclyl, wherein the alkyl, alkenyl, alkynyl, heteroalkyl, heteroalkenyl, heteroalkynyl, cycloalkyl and heterocyclyl are optionally substituted with one or more groups independently selected from hydroxyl, halogen, cyano, amino, alkyl and haloalkyl;
m is an integer from 0 to 5; and
n is an integer from 0 to 4.

In some embodiments, Ring A is aryl. In certain embodiments, Ring A is C₆₋₁₂ aryl, C₆₋₁₁ aryl, C₆₋₁₀ aryl, C₆₋₉ aryl, or C₆₋₈ aryl. In certain embodiments, Ring A is phenyl.

In some embodiments, Ring A is heteroaryl. In certain embodiments, Ring A is 5-12 membered heteroaryl, 5-11 membered heteroaryl, 5-10 membered heteroaryl, 5-9 membered heteroaryl, 5-8 membered heteroaryl, 5-7 membered heteroaryl or 5-6 membered heteroaryl.

In certain embodiments, Ring A is selected from a group consisting of furanyl, thiophenyl, pyrrolyl, pyridinyl, pyranyl, pyrimidinyl, pyridazinyl, pyrazinyl and tetrahydroisoquinolinyl.

In certain embodiments, Ring A is selected from a group consisting of

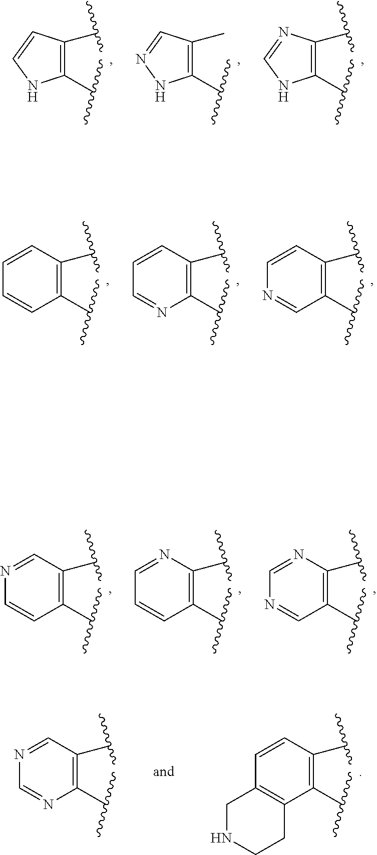

In some embodiments, Ring B is aryl. In certain embodiments, Ring B is C₆₋₁₂ aryl, C₆₋₁₁ aryl, C6-10 aryl, C₆₋₉ aryl, or C6-8 aryl. In certain embodiments, Ring B is phenyl.

In some embodiments, Ring B is heteroaryl. In certain embodiments, Ring B is 5-12 membered heteroaryl, 5-11 membered heteroaryl, 5-10 membered heteroaryl, 5-9 membered heteroaryl, 5-8 membered heteroaryl, 5-7 membered heteroaryl, or 5-6 membered heteroaryl. In certain embodiments, Ring B is pyridinyl or pyrazolyl.

In certain embodiments, Ring B is selected from a group consisting of

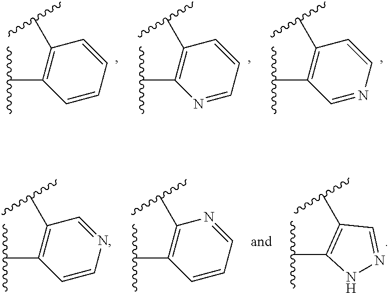

In some embodiments, L¹ is a bond.

In some embodiments, L¹ is alkyl. In certain embodiments, L¹ is C₁₋₆ alkyl, C₁₋₅ alkyl, C₁₋₄ alkyl, C₁₋₃ alkyl or C₁₋₂ alkyl.

In certain embodiments, L is selected from a group consisting of

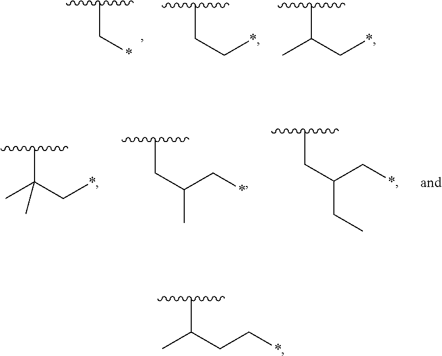

and *, wherein * end of L¹ is connected to L².

In some embodiments, L² is a bond.

In some embodiments, L² is N(R^{A}), and R^{A} is selected from alkyl or heterocyclyl, wherein the alkyl or heterocyclyl are are optionally substituted with one or more halogen or alkyl. In certain embodiments, L² is N(R^{A}), and R^{A} is ethyl, difluoroethyl, trifluoroethyl or oxetanyl.

In some embodiments, L² is cycloalkyl optionally substituted with one or more R^{B}. In certain embodiments, L² is C₃₋₁₀ cycloalkyl, C₃₋₉ cycloalkyl, C₃₋₈ cycloalkyl, C3.7 cycloalkyl, C₃₋₆ cycloalkyl, or C₃₋₅ cycloalkyl, each optionally substituted with one or more R^{B}.

In certain embodiments, L² is

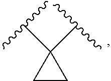 [SMILES: *C1(*)CC1]

which is optionally substituted with one or more R^{B}.

In some embodiments, L² is heterocyclyl optionally substituted with one or more R^{B}.

In certain embodiments, L² is heterocyclyl containing one or more heteroatoms selected from N, O or S. In certain embodiments, L² is 3-12 membered heterocyclyl, 3-11 membered heterocyclyl, 3-10 membered heterocyclyl, or 3-9 membered heterocyclyl, each optionally substituted with one or more R^{B}.

In certain embodiments, the L² is heterocyclyl selected from a group consisting of

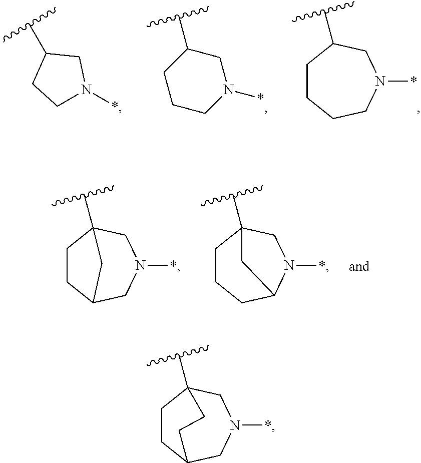

each of which is optionally substituted with one or more R^{B}, wherein * end of L² is connected to L³.

In some embodiments, L² is cycloalkyl or heterocyclyl substituted with one or more R^{B}, and R^{B} is alkyl. In certain embodiments, R^{B} is C₁₋₆ alkyl, C₁₋₅ alkyl, C₁₋₄ alkyl, C₁₋₃ alkyl or C₁₋₂ alkyl. In certain embodiments, R^{B} is methyl.

In some embodiments, L² is selected from a group consisting of:

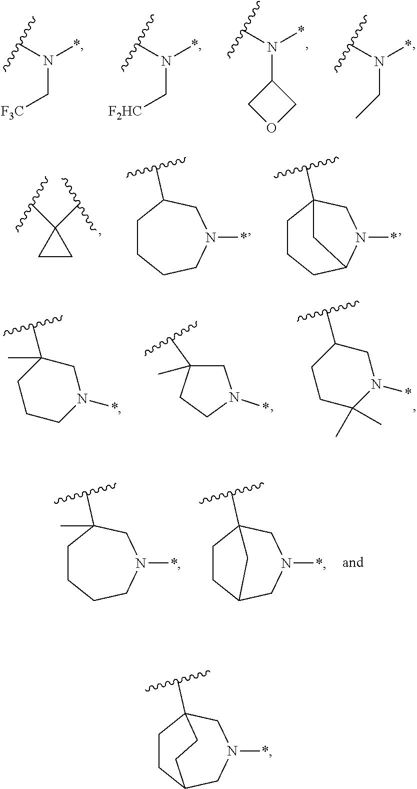

wherein * end of L² is connected to L³.

In some embodiments, L³ is alkyl. In some embodiments, L³ is C₁₋₆ alkyl, C₁₋₅ alkyl, C₁₋₄ alkyl, C₁₋₃ alkyl or C₁₋₂ alkyl.

In certain embodiments, L³ is ethyl.

In some embodiments, L⁴ is O or NH.

In some embodiments, L¹ is a bond and L² is a bond or heterocyclyl optionally substituted with one or more R^{B}.

In some embodiments, L¹ is alkyl and L² is a bond, N(R^{A}), or cycloalkyl optionally substituted with one or more R^{B}.

In some embodiments, -L¹-L²-L³-L⁴- is -alkyl-O—. In certain embodiments, -L¹-L²-L³-L⁴- is (C₃₋₈ alkyl)-O—. In certain embodiments, -L¹-L²-L³-L⁴- is —CH₂CH(CH₃)(CH₂)₃—O— or —CH₂CH(CH₂CH₃)(CH₂)₃—O—.

In some embodiments, -L¹-L²-L³-L⁴- is -heterocyclyl-alkyl-O—, wherein the heterocyclyl is optionally substituted with one or more R^{B}. In certain embodiments, -L¹-L²-L³-L⁴- is -(5-10 membered heterocyclyl)-(C₁₋₆ alkyl)-O—, wherein the 5-10 membered heterocyclyl is optionally substituted with one or more R^{B}. In certain embodiments, -L¹-L²-L³-L⁴- is selected from the group consisting of:

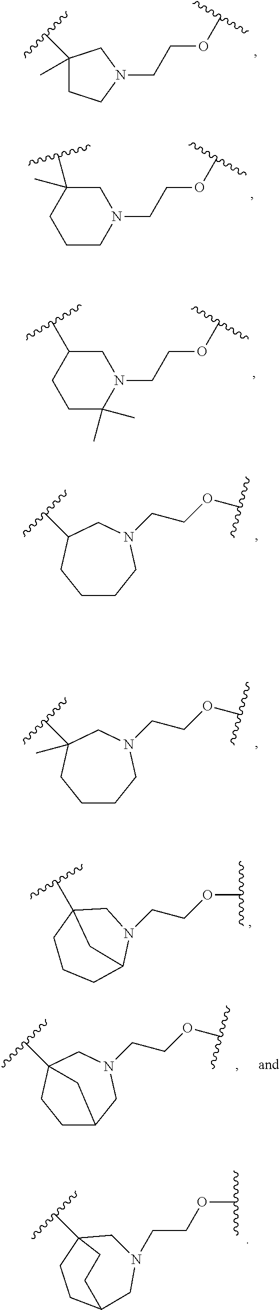

In some embodiments, -L¹-L²-L³-L⁴- is -alkyl-N(R^{A})-alkyl-O—, wherein R^{A} is selected from alkyl or heterocyclyl, wherein the alkyl or heterocyclyl are are optionally substituted with one or more halogen or alkyl. In certain embodiments, -L¹-L²-L³-L⁴- is —(C₁₋₆ alkyl)-N(R^{A})—(C₁₋₆ alkyl)-O—, wherein R^{A} is selected from C₁₋₆ alkyl or 3- to 6-membered heterocyclyl, wherein the alkyl or heterocyclyl are are optionally substituted with one or more halogen or

C₁₋₆ alkyl. In certain embodiments, -L¹-L²-L³-L⁴- is

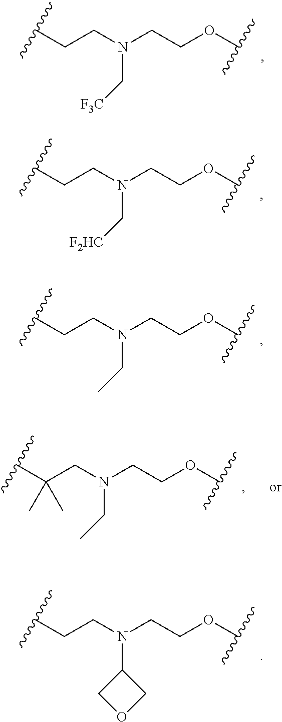

In some embodiments, -L¹-L²-L³-L⁴- is -alkyl-cycloalkyl-alkyl-O—, wherein the cycloalkyl is optionally substituted with one or more R^{B}. In certain embodiments, -L¹-L²-L³-L⁴- is —(C₁₋₆ alkyl)-(C₃₋₆ cycloalkyl)-(C₁₋₆ alkyl)-O—, wherein the C₃₋₆ cycloalkyl is optionally substituted with one or more R^{B}. In certain embodiments, -L¹-L²-L³-L⁴- is

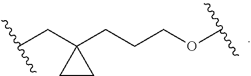 [SMILES: *CC1(CCCO*)CC1]

In some embodiments, R¹ is hydroxy and m is 1.

In some embodiments, R¹ is halogen and m is 1. In certain embodiments, R¹ is bromo or fluoro and m is 1.

In some embodiments, R¹ is —N(R^{D})₂ and m is 1.

In some embodiments, R¹ is —N(R^{D})₂, and each R^{D} is independently hydrogen, or alkyl optionally substituted with one or more groups independently selected from —N(R^{F})₂ or —OR^{G}.

In certain embodiments, R¹ is —N(R^{D})₂, and each R^{D} is C₁₋₆ alkyl, C₁₋₅ alkyl, C₁₋₄ alkyl, C₁₋₃ alkyl or C₁₋₂ alkyl optionally substituted with one or more groups independently selected from —N(R^{F})₂ or —OR^{G}. In certain embodiments, R¹ is —N(R^{D})₂, and each R^{D} is independently methyl, methoxyethyl, N,N-dimethylaminoethyl, hydroxyethyl or N,N-dimethylaminopropyl.

In some embodiments, R¹ is heterocyclyl optionally substituted with one or more R^{E} and m is 1. In certain embodiments, R¹ is 3- to 10-membered heterocyclyl optionally substituted with one or more R^{E} and m is 1.

In certain embodiments, R¹ is selected from a group consisting of:

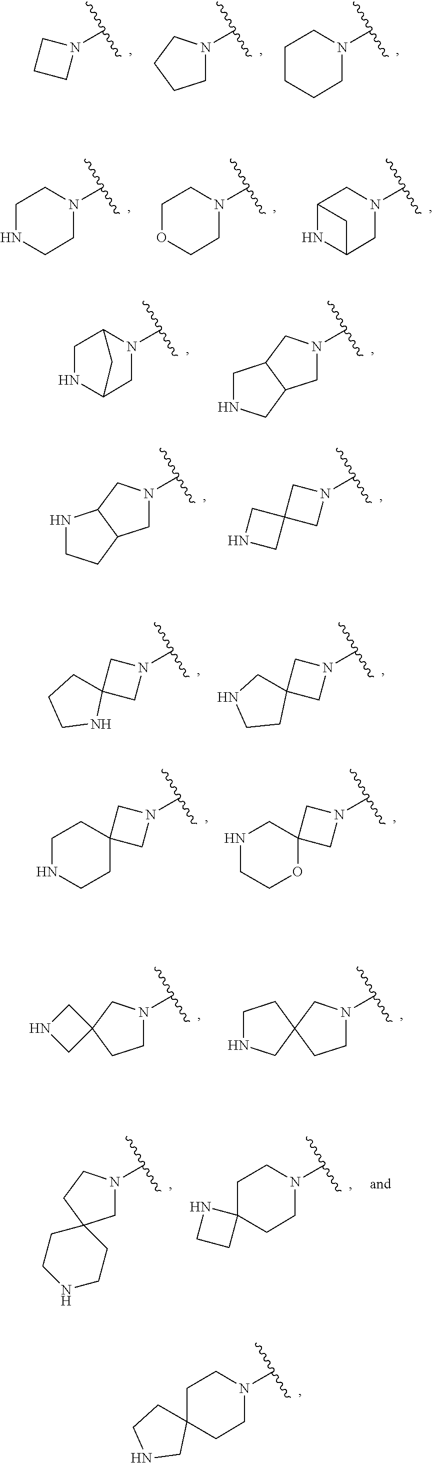

each of which is optionally substituted with one or more R^{E}.

In some embodiments, R^{E} is halogen.

In certain embodiments, R^{E} is F.

In certain embodiments, each R^{E} is independently halogen, —N(R^{F})₂, -alkyl-N(R^{F})₂, —C(O)OR^{G}, or alkyl optionally substituted with one or more halogen.

In some embodiments, each of R^{F} and R^{G} is independently alkyl. In certain embodiments, each of R^{F} and R^{G} is independently C₁₋₆ alkyl, C₁₋₅ alkyl, C₁₋₄ alkyl, C₁₋₃ alkyl or C₁₋₂ alkyl.

In certain embodiments, each of R^{F} and R^{G} is independently C₁₋₃ alkyl.

In certain embodiments, each R^{E} is independently selected from a group consisting of F, —N(CH₃)₂, —C₁₋₃ alkyl-N(CH₃)₂, —C(O)O(tert-butyl), methyl, ethyl, or trifluoroethyl.

In certain embodiments, R^{E} is cycloalkyl or heterocyclyl, wherein the cycloalkyl and heterocyclyl are optionally substituted with one or more alkyl and/or halogen.

In certain embodiments, R^{E} is selected from cyclopropyl, morpholine, piperazine, oxetyl or azetidinyl, each of which is optionally substituted with one or more alkyl and/or halogen.

In certain embodiments, R^{E} is selected from cyclopropyl, morpholinyl, piperazinyl, oxetyl or azetidinyl, each of which is optionally substituted with one or more alkyl and/or halogen.

In some embodiments, m is 2, one of R¹ is halogen, and the other R¹ is heterocyclyl optionally substituted with one or more R^{E}.

In certain embodiments, m is 2, one of R¹ is halogen, and the other R¹ is heterocyclyl selected from the group consisting of

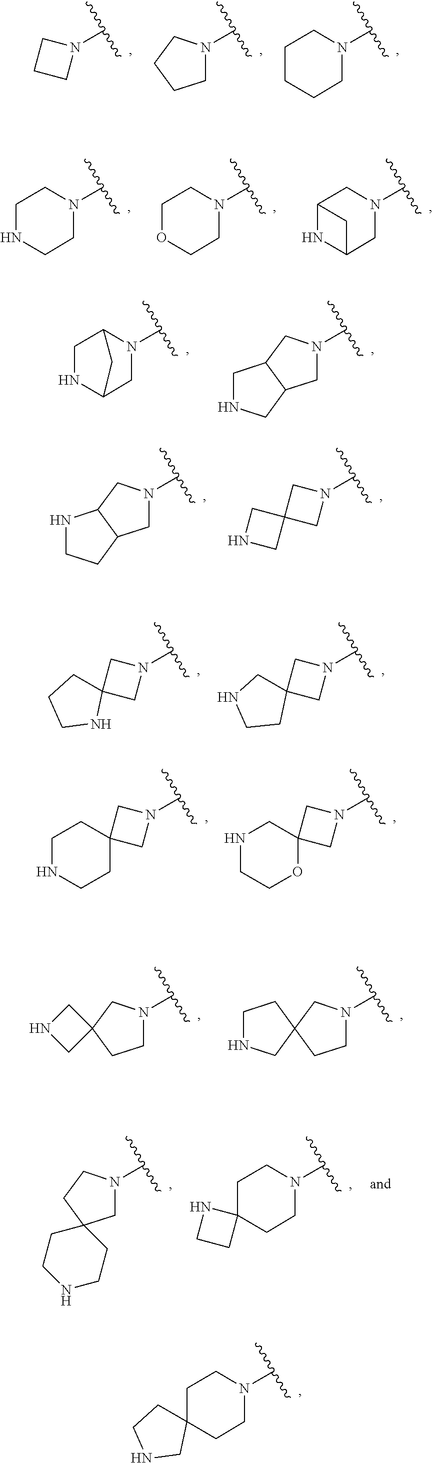

each of which is optionally substituted with one or more R^{E}.

In certain embodiments, each R^{E} is independently selected from halogen, —N(R^{F})₂, -alkyl-N(R^{F})₂, —C(O)OR^{G}, or alkyl optionally substituted with one or more halogen.

In some embodiments, R¹ is selected from a group consisting of:

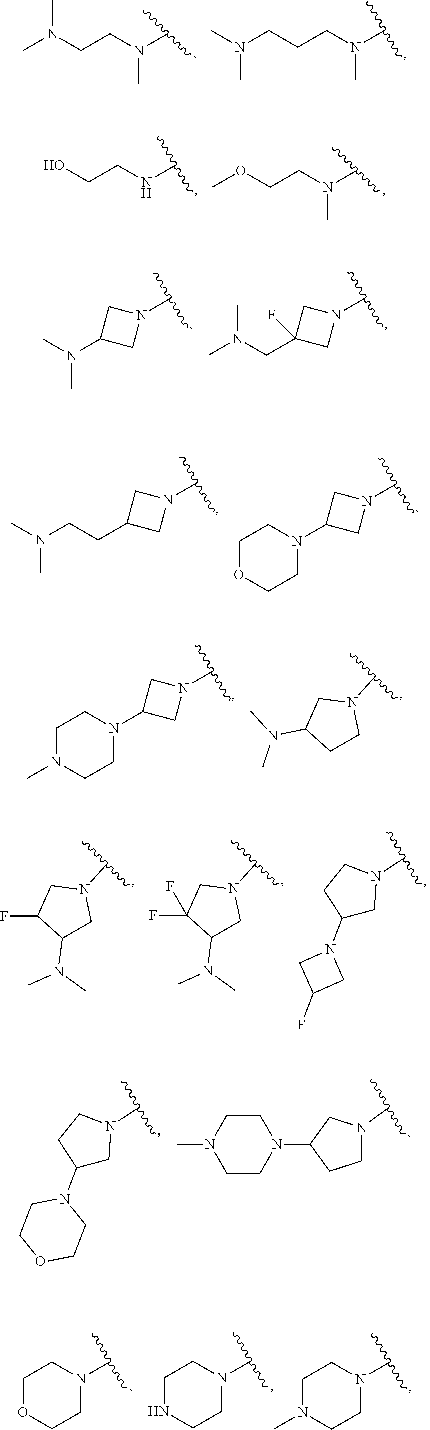

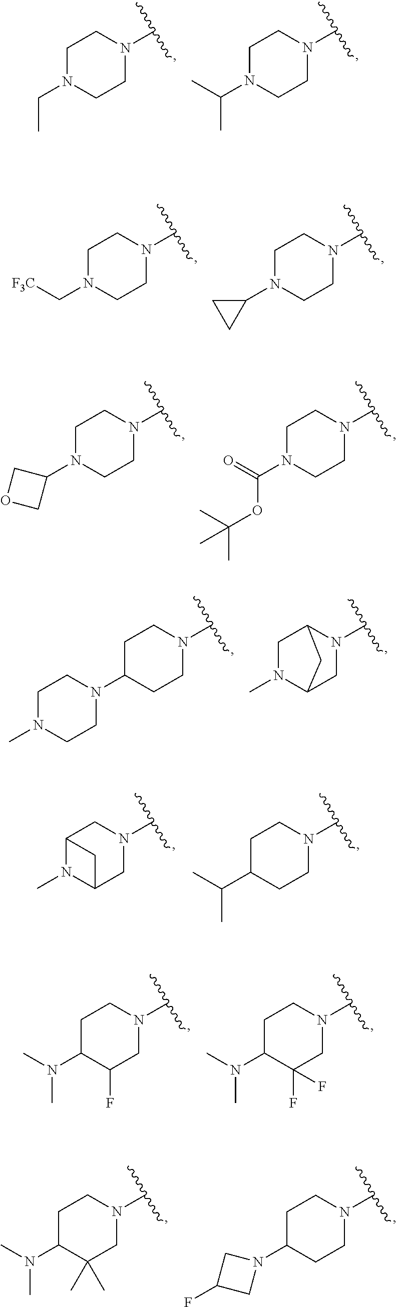

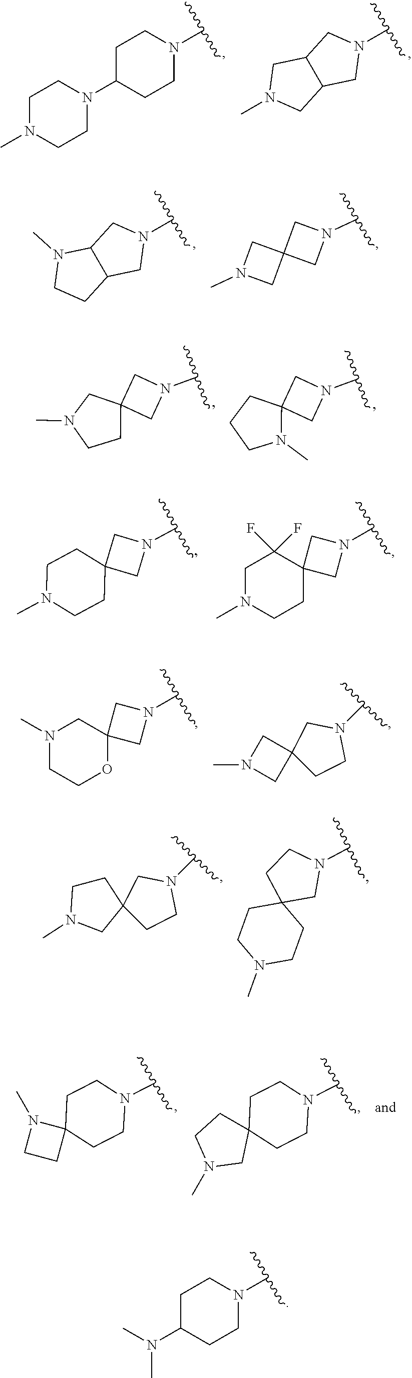

In certain embodiments, R² is halogen and n is 1 or 2.

In certain embodiments, R² is F.

In some embodiments, R² is alkyl or cycloalkyl, wherein the alkyl and cycloalkyl are optionally substituted with one or more halogen. In certain embodiments, R² is C₁₋₆ alkyl or C₃₋₆ cycloalkyl, wherein the C₁₋₆ alkyl and C₃₋₆ cycloalkyl are optionally substituted with one or more halogen. In certain embodiments, R² is methyl, ethyl, cyclopropyl or trifluoroethyl.

In some embodiments, R³ is alkyl or cycloalkyl. In certain embodiments, R³ is C₁₋₆ alkyl or C₃₋₆ cycloalkyl. In certain embodiments, R³ is methyl, ethyl or cyclopropyl.

In some embodiments, m is 0, 1 or 2.

In some embodiments, n is 0 or 1.

Exemplary compounds of Formula (I) are set forth below.

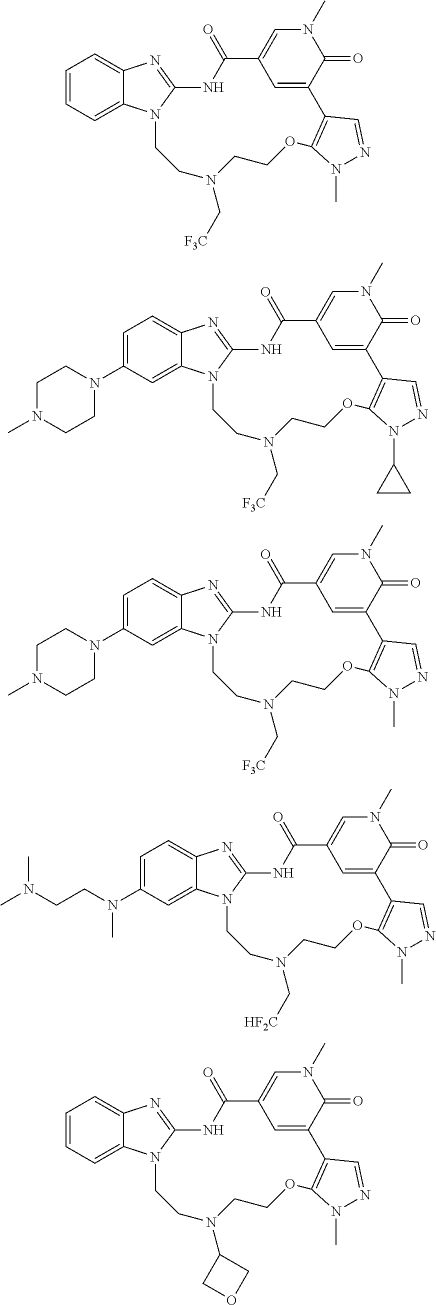

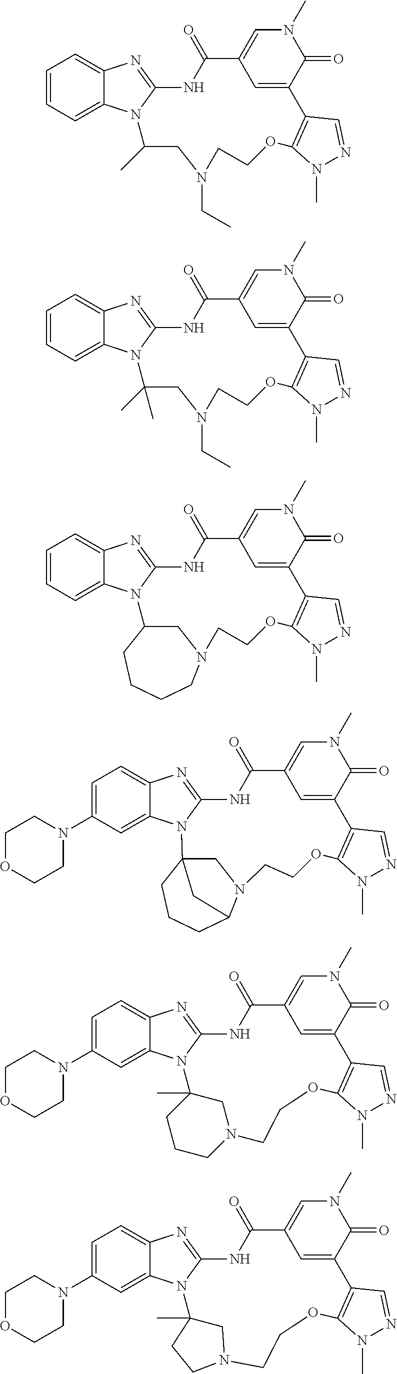

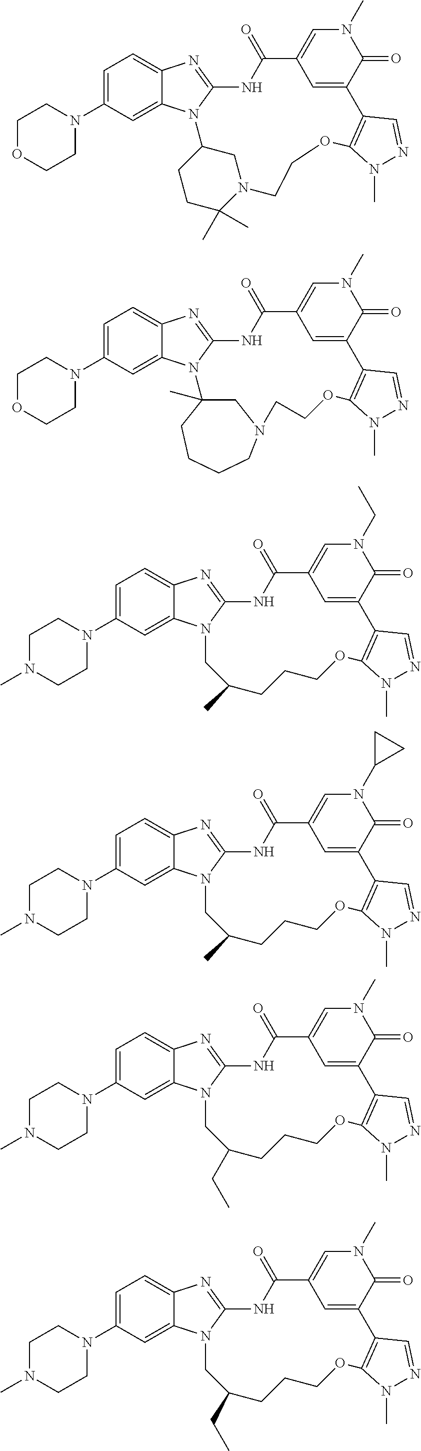

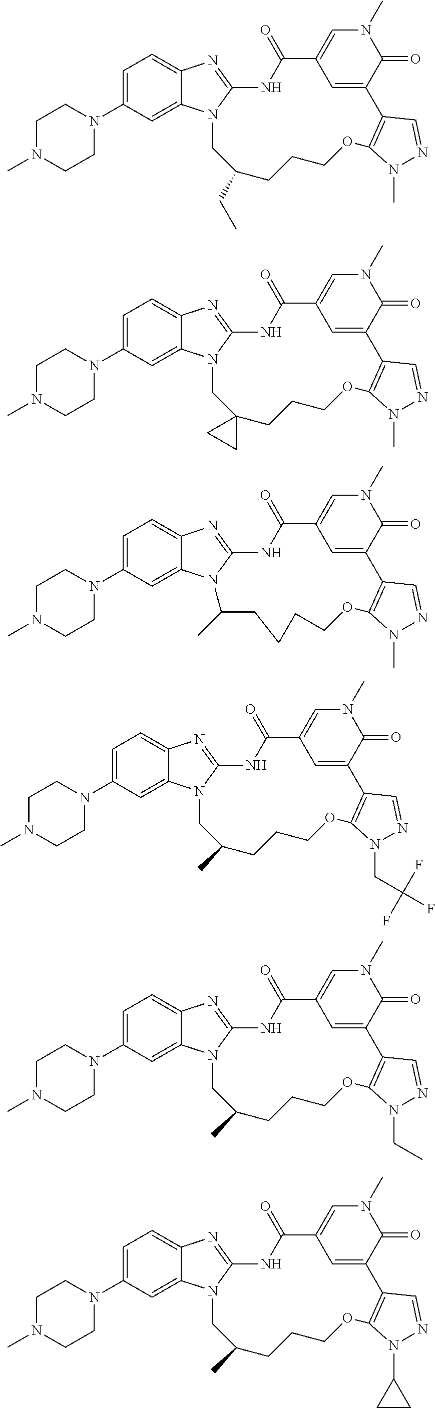

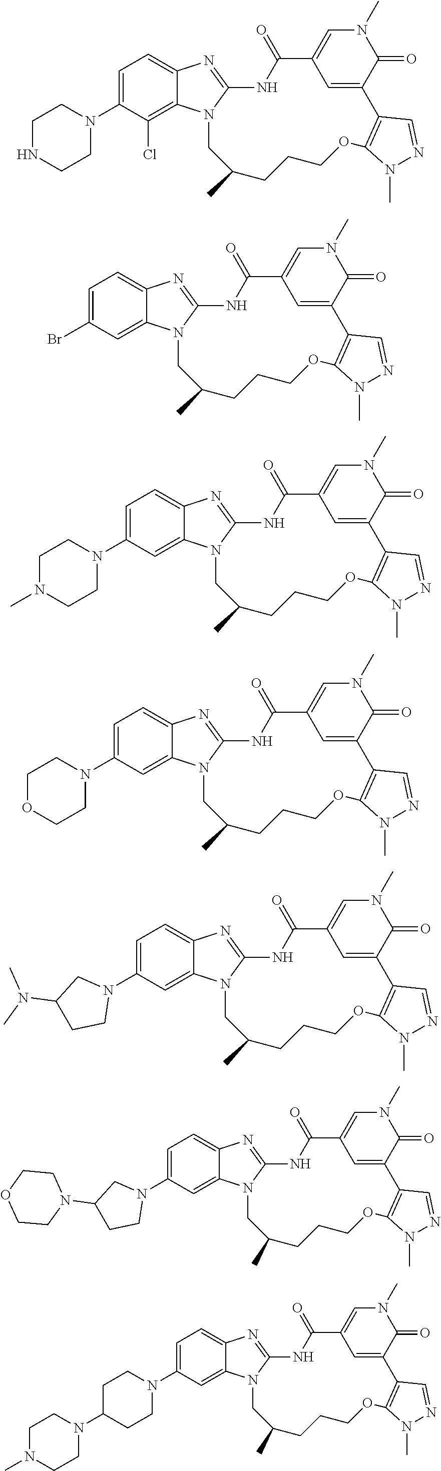

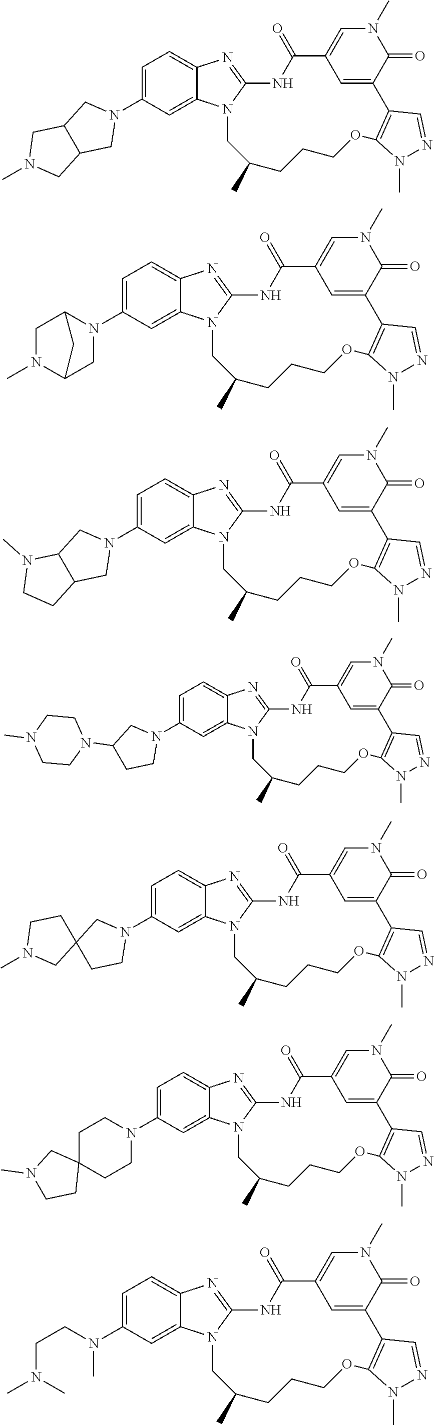

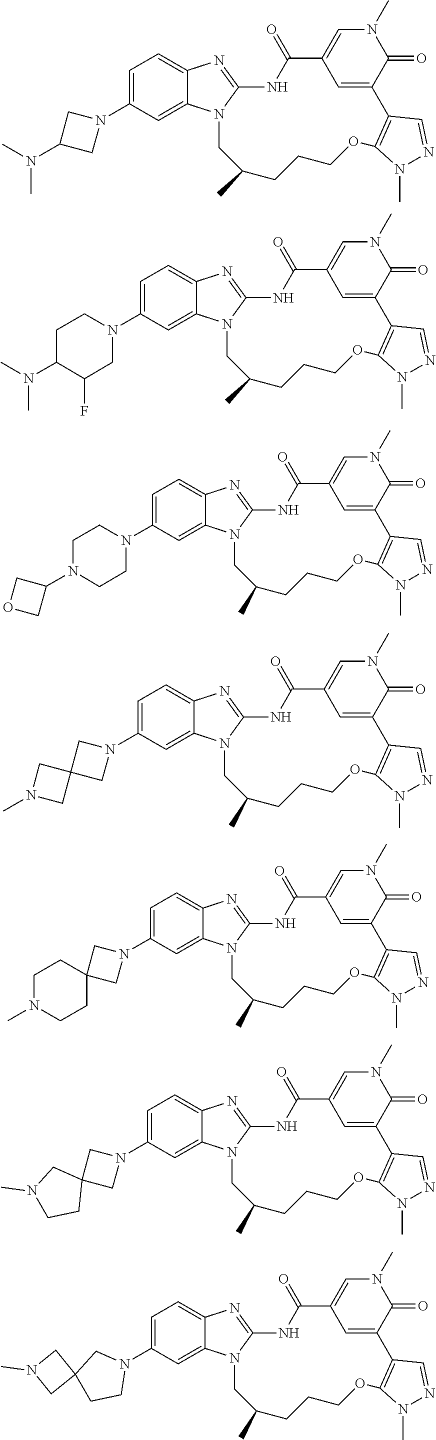

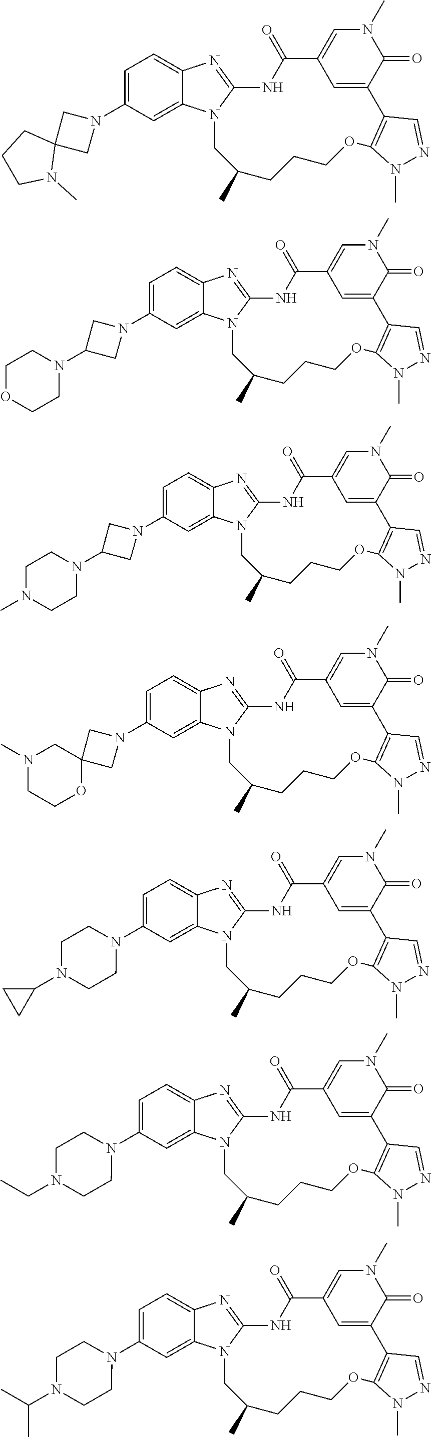

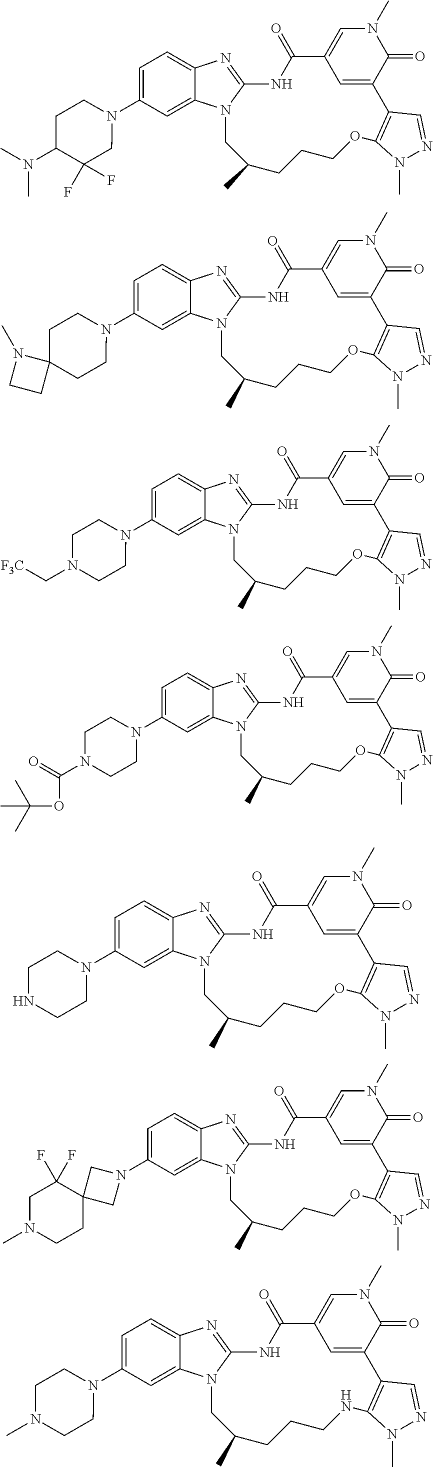

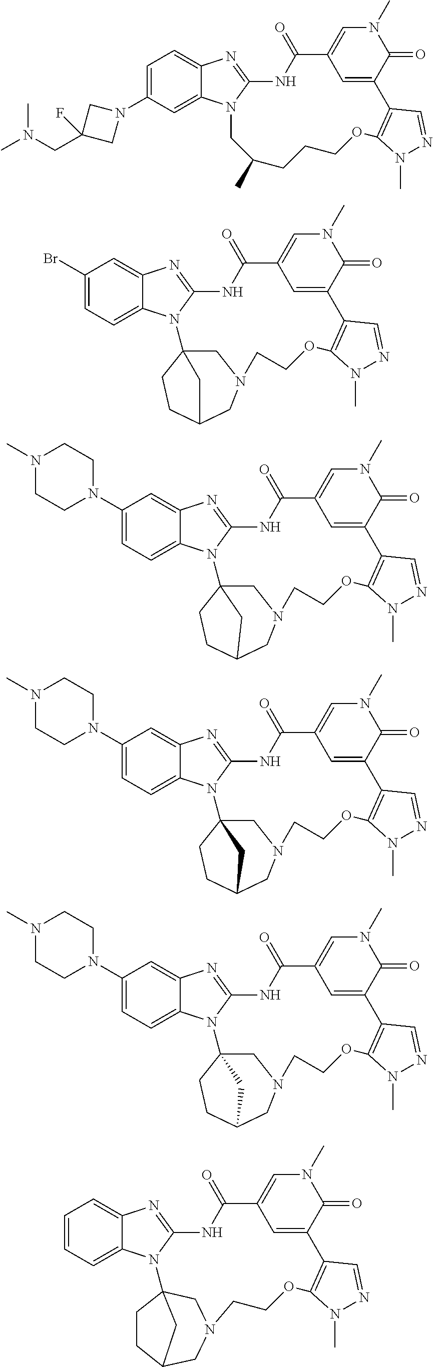

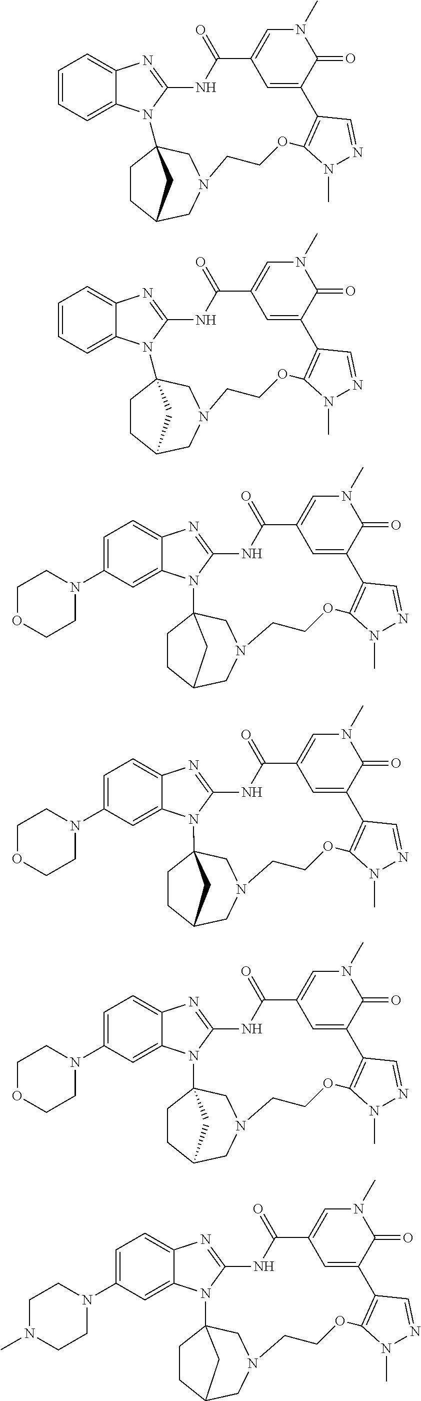

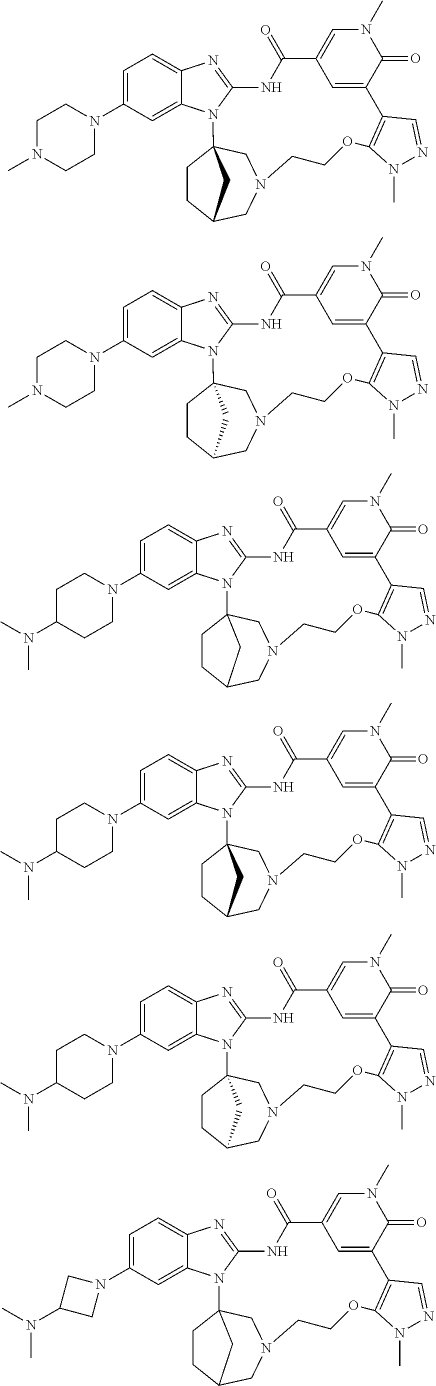

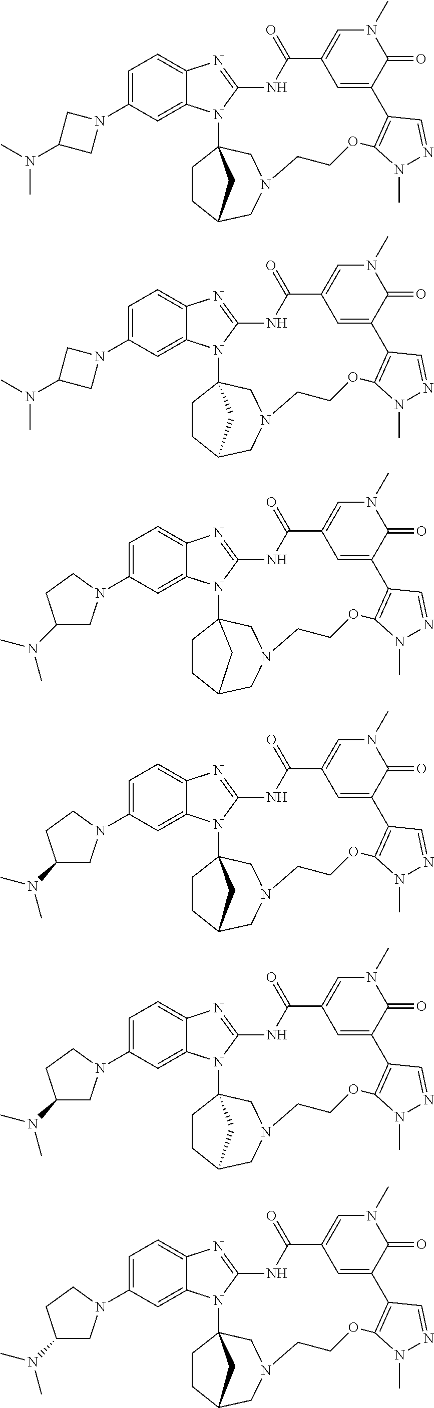

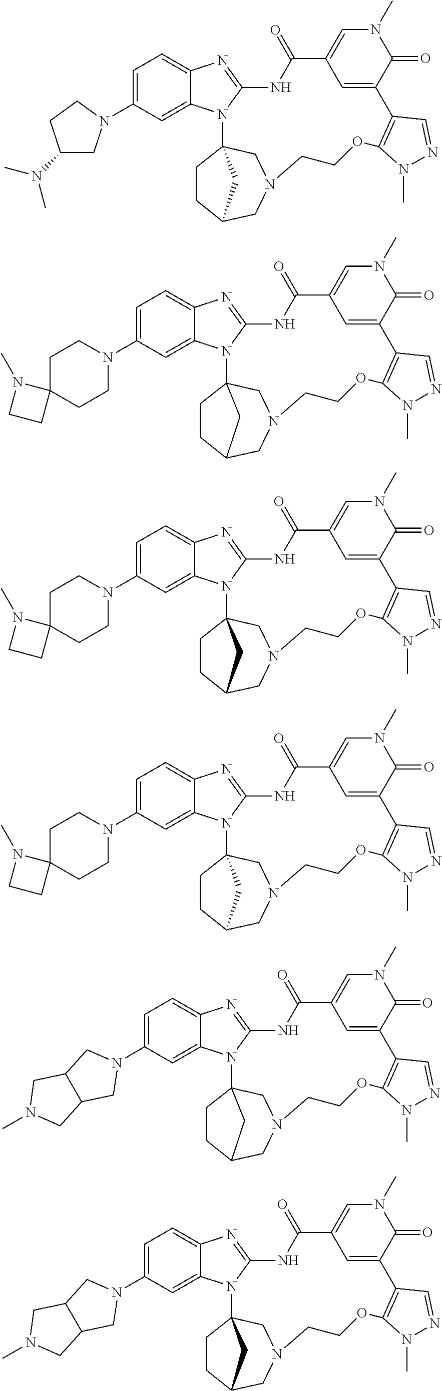

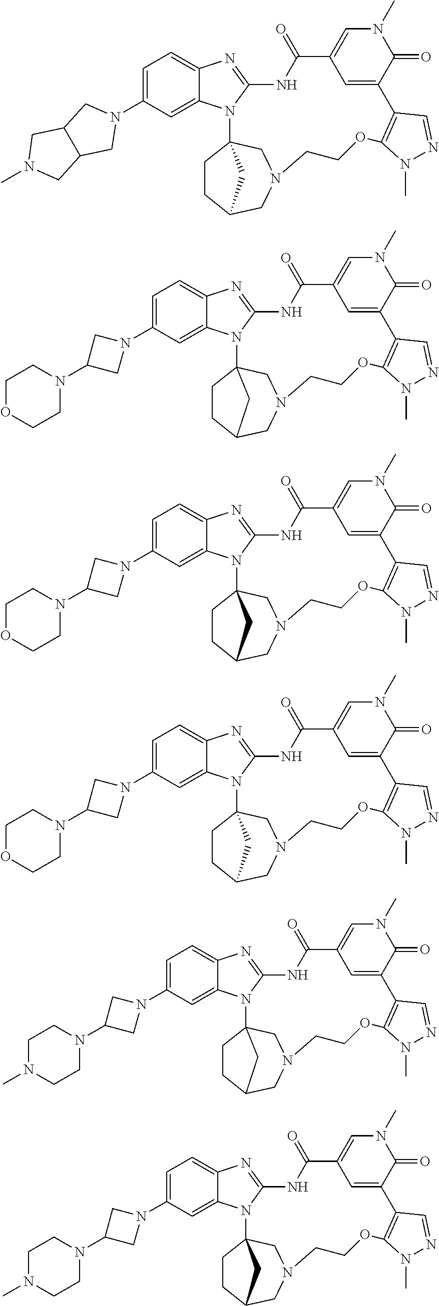

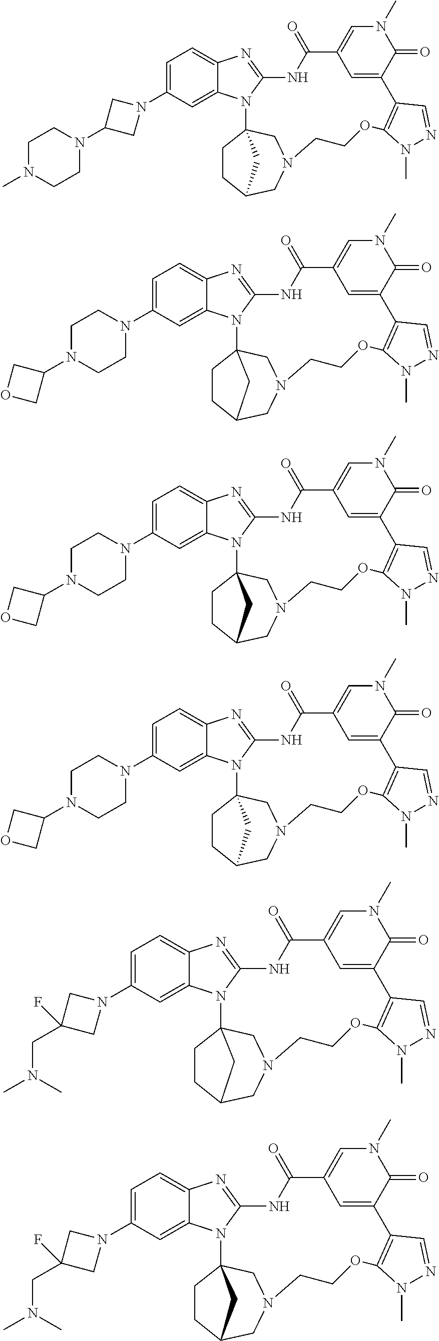

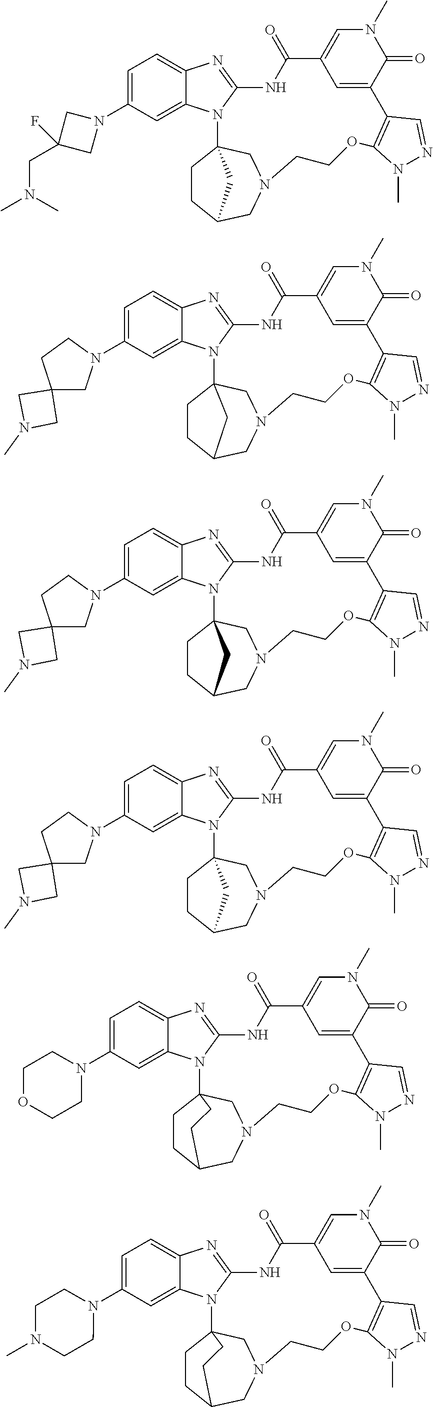

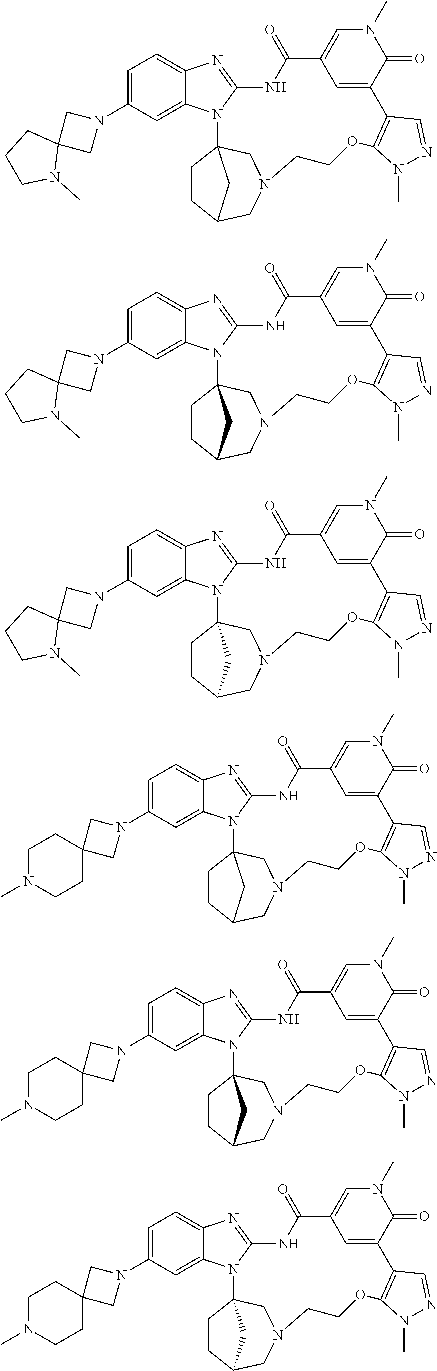

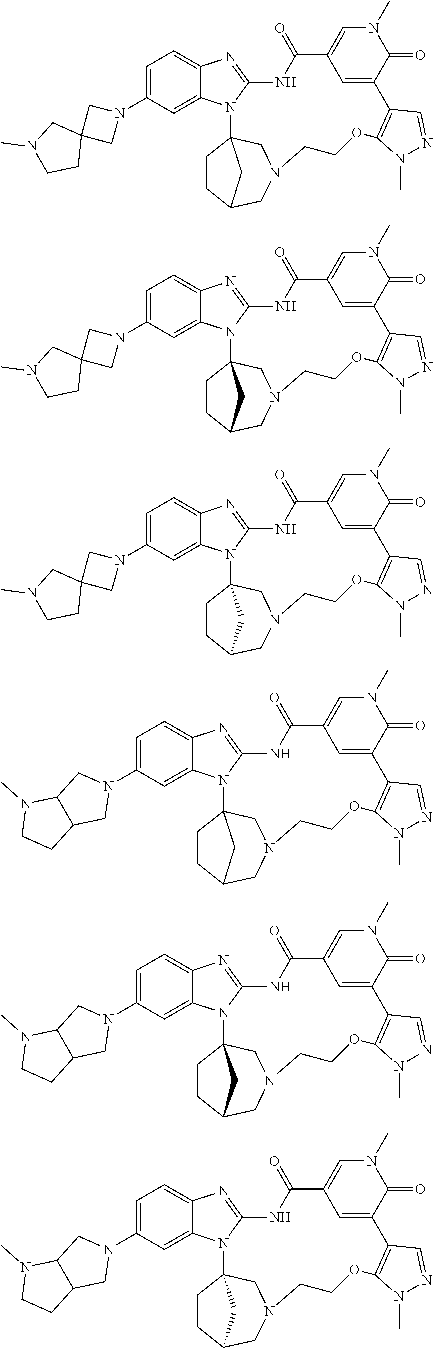

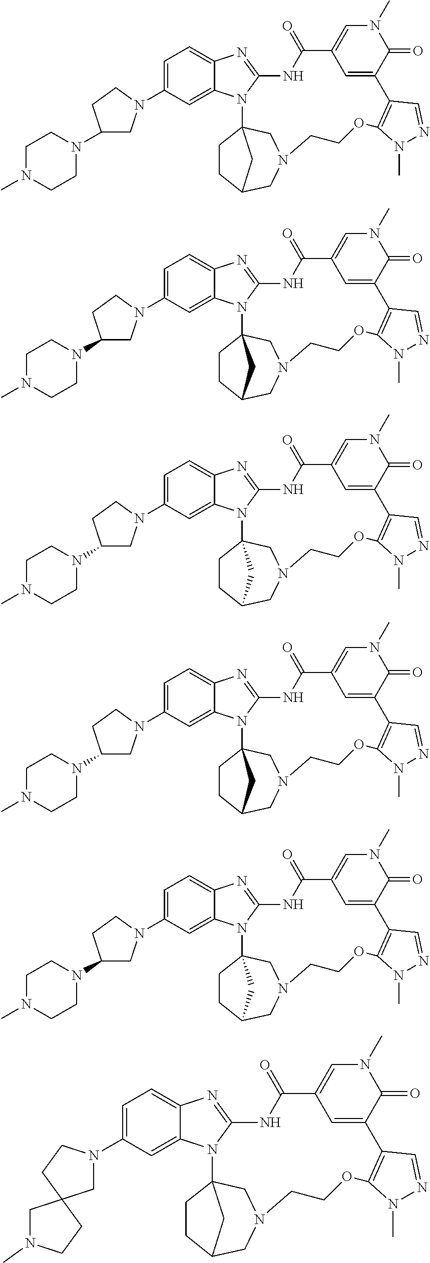

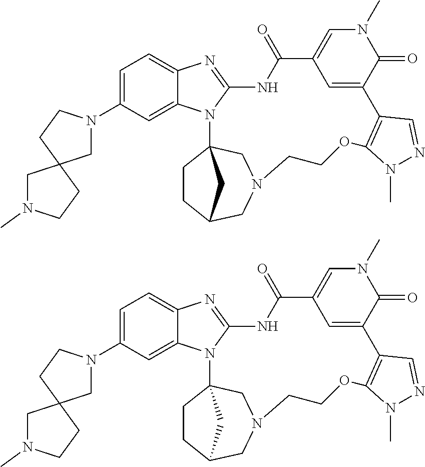

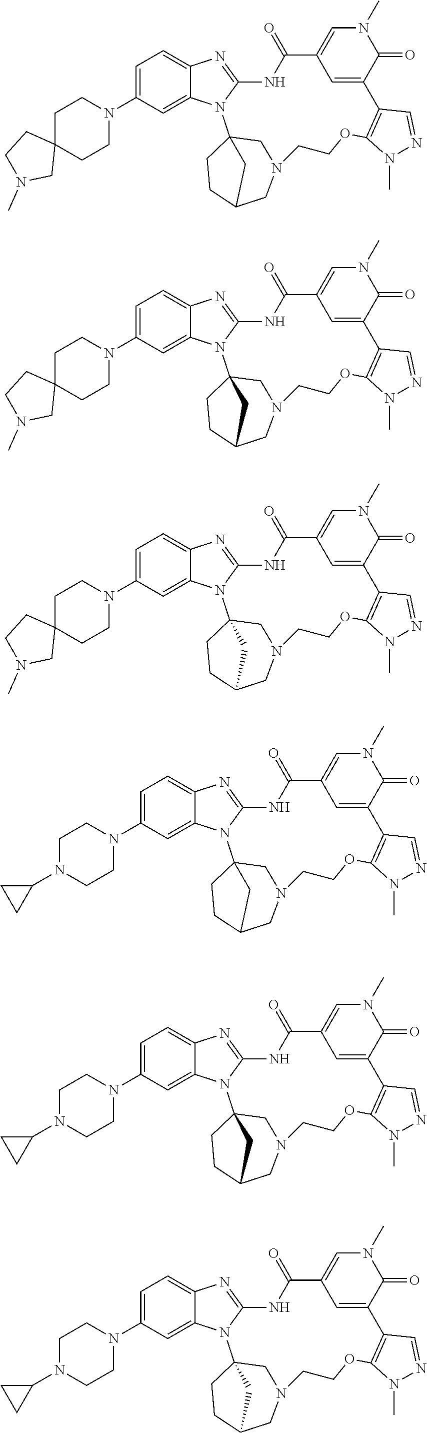

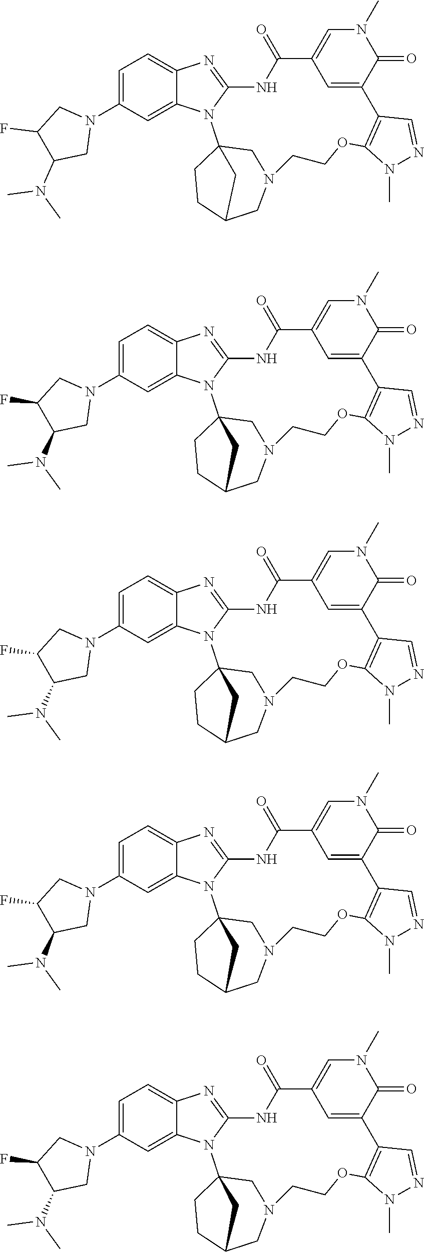 [SMILES: CN(C)C1CN(c2ccc3nc4n(c3c2)C23CCC(CN(CCOc5c(cnn5C)-c5cc(cn(C)c5=O)C(=O)N4)C2)C3)CC1F ; CN(C)[C@@H]1CN(c2ccc3nc4n(c3c2)[C@@]23CC[C@@H](CN(CCOc5c(cnn5C)-c5cc(cn(C)c5=O)C(=O)N4)C2)C3)C[C@@H]1F ; CN(C)[C@H]1CN(c2ccc3nc4n(c3c2)[C@@]23CC[C@@H](CN(CCOc5c(cnn5C)-c5cc(cn(C)c5=O)C(=O)N4)C2)C3)C[C@H]1F ; CN(C)[C@@H]1CN(c2ccc3nc4n(c3c2)[C@@]23CC[C@@H](CN(CCOc5c(cnn5C)-c5cc(cn(C)c5=O)C(=O)N4)C2)C3)C[C@H]1F ; CN(C)[C@H]1CN(c2ccc3nc4n(c3c2)[C@@]23CC[C@@H](CN(CCOc5c(cnn5C)-c5cc(cn(C)c5=O)C(=O)N4)C2)C3)C[C@@H]1F]

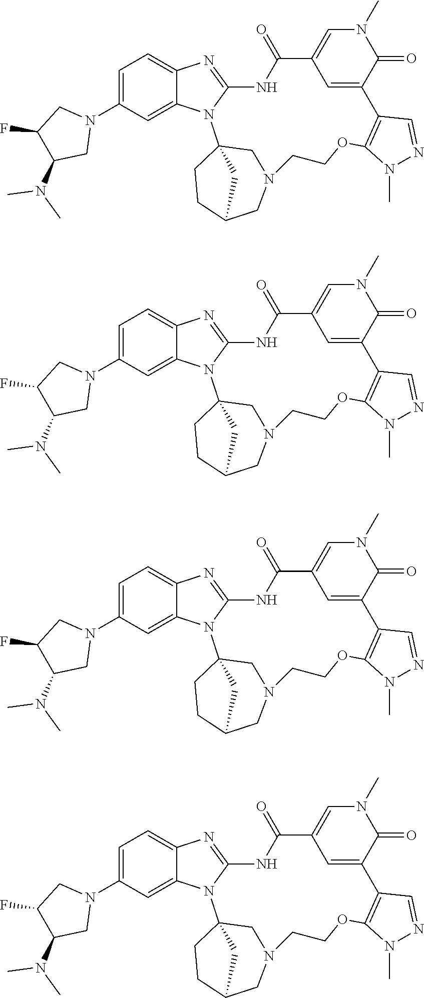 [SMILES: CN(C)[C@@H]1CN(c2ccc3nc4n(c3c2)[C@]23CC[C@H](CN(CCOc5c(cnn5C)-c5cc(cn(C)c5=O)C(=O)N4)C2)C3)C[C@@H]1F ; CN(C)[C@H]1CN(c2ccc3nc4n(c3c2)[C@]23CC[C@H](CN(CCOc5c(cnn5C)-c5cc(cn(C)c5=O)C(=O)N4)C2)C3)C[C@H]1F ; CN(C)[C@H]1CN(c2ccc3nc4n(c3c2)[C@]23CC[C@H](CN(CCOc5c(cnn5C)-c5cc(cn(C)c5=O)C(=O)N4)C2)C3)C[C@@H]1F ; CN(C)[C@@H]1CN(c2ccc3nc4n(c3c2)[C@]23CC[C@H](CN(CCOc5c(cnn5C)-c5cc(cn(C)c5=O)C(=O)N4)C2)C3)C[C@H]1F]

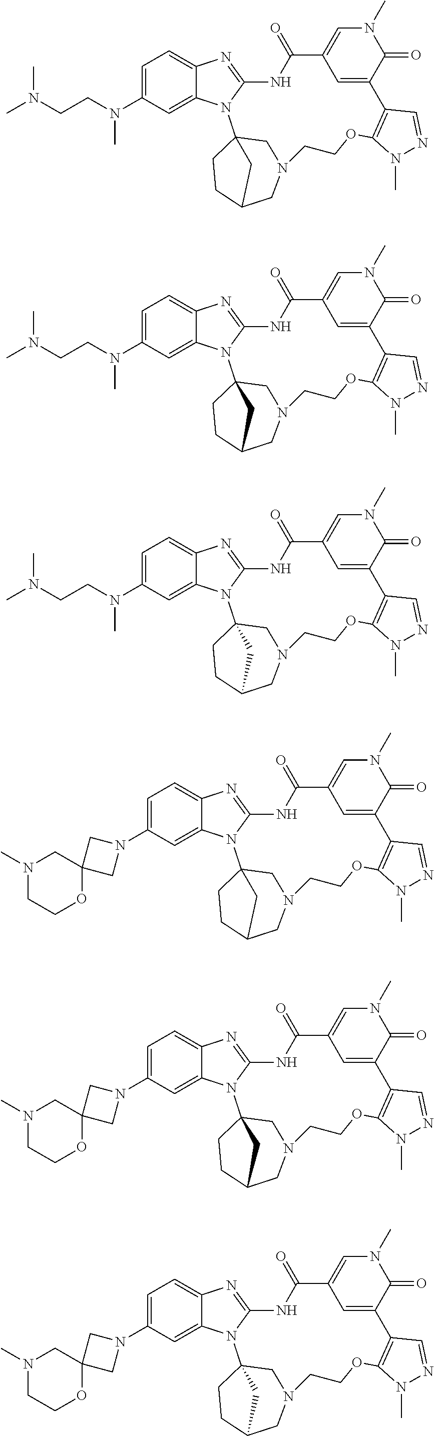

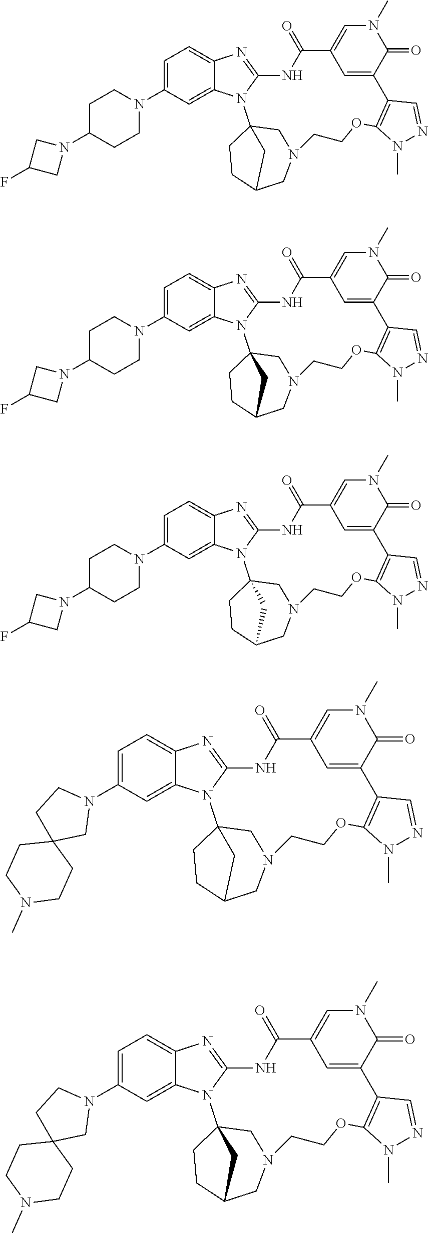 [SMILES: Cn1ncc2c1OCCN1CC3CCC(C3)(C1)n1c(nc3ccc(N4CCC(N5CC(F)C5)CC4)cc31)NC(=O)c1cc-2c(=O)n(C)c1 ; Cn1ncc2c1OCCN1C[C@@H]3CC[C@@](C3)(C1)n1c(nc3ccc(N4CCC(N5CC(F)C5)CC4)cc31)NC(=O)c1cc-2c(=O)n(C)c1 ; Cn1ncc2c1OCCN1C[C@H]3CC[C@](C3)(C1)n1c(nc3ccc(N4CCC(N5CC(F)C5)CC4)cc31)NC(=O)c1cc-2c(=O)n(C)c1 ; CN1CCC2(CC1)CCN(c1ccc3nc4n(c3c1)C13CCC(CN(CCOc5c(cnn5C)-c5cc(cn(C)c5=O)C(=O)N4)C1)C3)C2 ; CN1CCC2(CC1)CCN(c1ccc3nc4n(c3c1)[C@@]13CC[C@@H](CN(CCOc5c(cnn5C)-c5cc(cn(C)c5=O)C(=O)N4)C1)C3)C2]

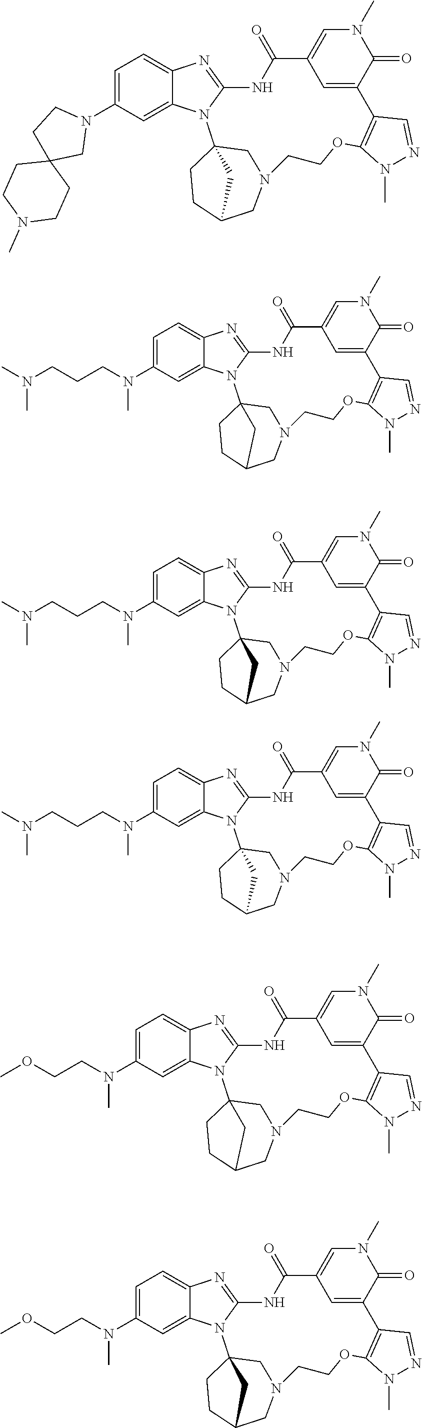

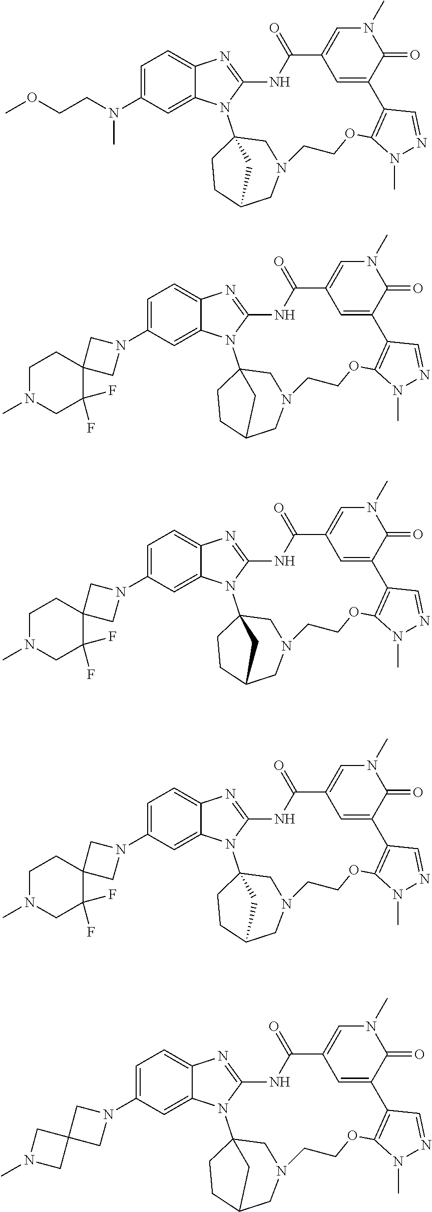 [SMILES: COCCN(C)c1ccc2nc3n(c2c1)[C@]12CC[C@H](CN(CCOc4c(cnn4C)-c4cc(cn(C)c4=O)C(=O)N3)C1)C2 ; CN1CCC2(CN(c3ccc4nc5n(c4c3)C34CCC(CN(CCOc6c(cnn6C)-c6cc(cn(C)c6=O)C(=O)N5)C3)C4)C2)C(F)(F)C1 ; CN1CCC2(CN(c3ccc4nc5n(c4c3)[C@@]34CC[C@@H](CN(CCOc6c(cnn6C)-c6cc(cn(C)c6=O)C(=O)N5)C3)C4)C2)C(F)(F)C1 ; CN1CCC2(CN(c3ccc4nc5n(c4c3)[C@]34CC[C@H](CN(CCOc6c(cnn6C)-c6cc(cn(C)c6=O)C(=O)N5)C3)C4)C2)C(F)(F)C1 ; CN1CC2(C1)CN(c1ccc3nc4n(c3c1)C13CCC(CN(CCOc5c(cnn5C)-c5cc(cn(C)c5=O)C(=O)N4)C1)C3)C2]

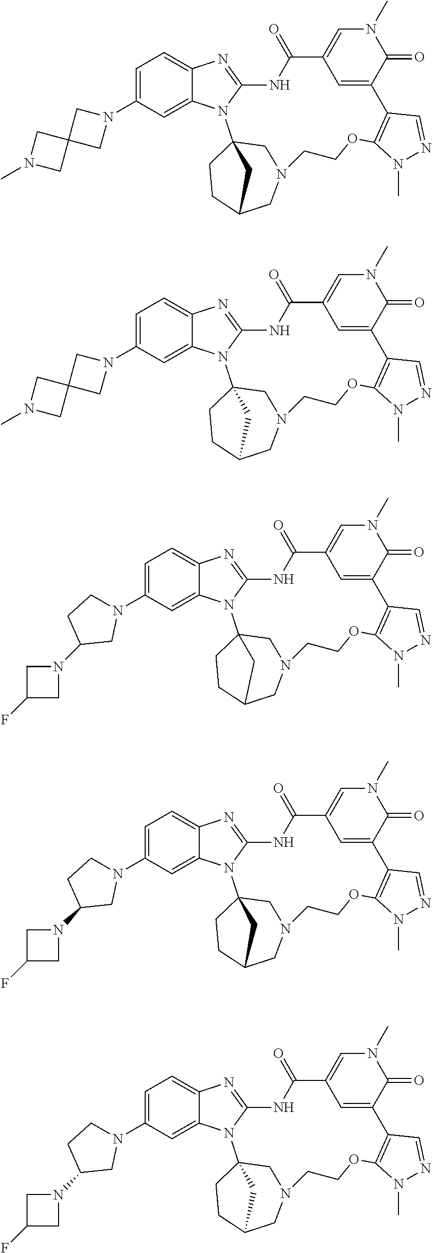 [SMILES: CN1CC2(C1)CN(c1ccc3nc4n(c3c1)[C@@]13CC[C@@H](CN(CCOc5c(cnn5C)-c5cc(cn(C)c5=O)C(=O)N4)C1)C3)C2 ; CN1CC2(C1)CN(c1ccc3nc4n(c3c1)[C@]13CC[C@H](CN(CCOc5c(cnn5C)-c5cc(cn(C)c5=O)C(=O)N4)C1)C3)C2 ; Cn1ncc2c1OCCN1CC3CCC(C3)(C1)n1c(nc3ccc(N4CCC(N5CC(F)C5)C4)cc31)NC(=O)c1cc-2c(=O)n(C)c1 ; Cn1ncc2c1OCCN1C[C@@H]3CC[C@@](C3)(C1)n1c(nc3ccc(N4CC[C@H](N5CC(F)C5)C4)cc31)NC(=O)c1cc-2c(=O)n(C)c1 ; Cn1ncc2c1OCCN1C[C@H]3CC[C@](C3)(C1)n1c(nc3ccc(N4CC[C@@H](N5CC(F)C5)C4)cc31)NC(=O)c1cc-2c(=O)n(C)c1]

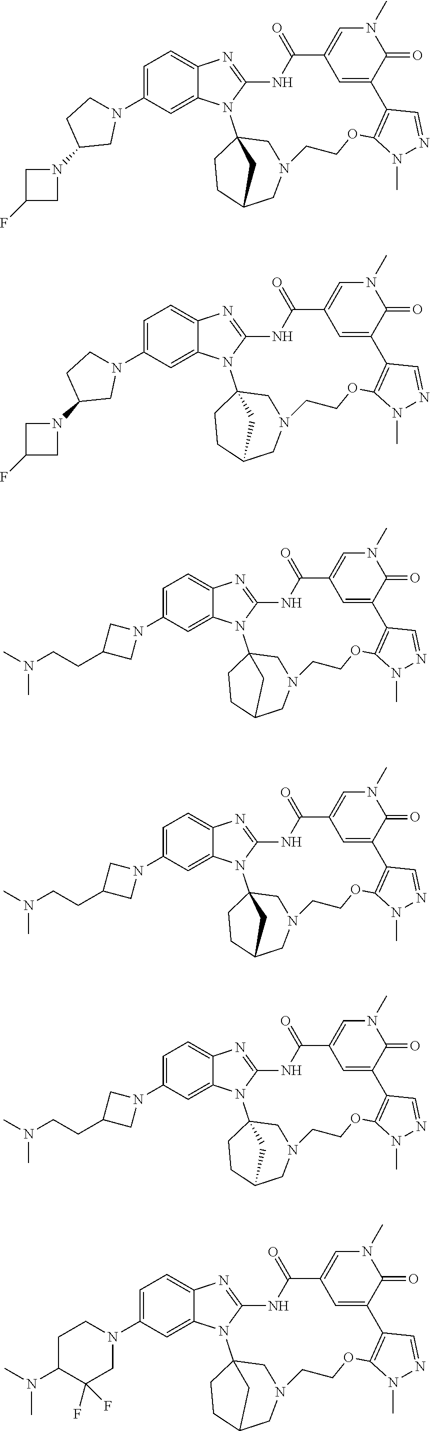

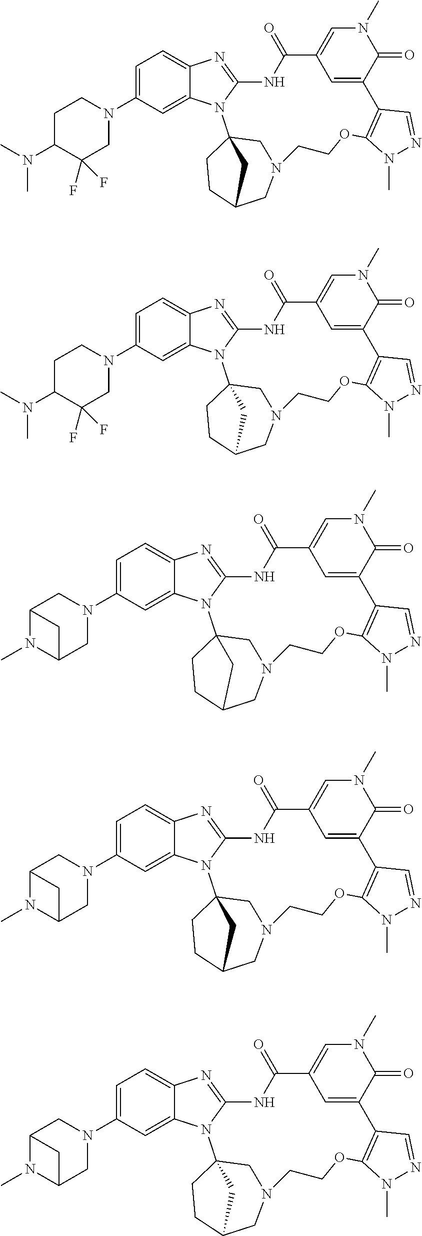 [SMILES: CN(C)C1CCN(c2ccc3nc4n(c3c2)[C@@]23CC[C@@H](CN(CCOc5c(cnn5C)-c5cc(cn(C)c5=O)C(=O)N4)C2)C3)CC1(F)F ; CN(C)C1CCN(c2ccc3nc4n(c3c2)[C@]23CC[C@H](CN(CCOc5c(cnn5C)-c5cc(cn(C)c5=O)C(=O)N4)C2)C3)CC1(F)F ; CN1C2CC1CN(c1ccc3nc4n(c3c1)C13CCC(CN(CCOc5c(cnn5C)-c5cc(cn(C)c5=O)C(=O)N4)C1)C3)C2 ; CN1C2CC1CN(c1ccc3nc4n(c3c1)[C@@]13CC[C@@H](CN(CCOc5c(cnn5C)-c5cc(cn(C)c5=O)C(=O)N4)C1)C3)C2 ; CN1C2CC1CN(c1ccc3nc4n(c3c1)[C@]13CC[C@H](CN(CCOc5c(cnn5C)-c5cc(cn(C)c5=O)C(=O)N4)C1)C3)C2]

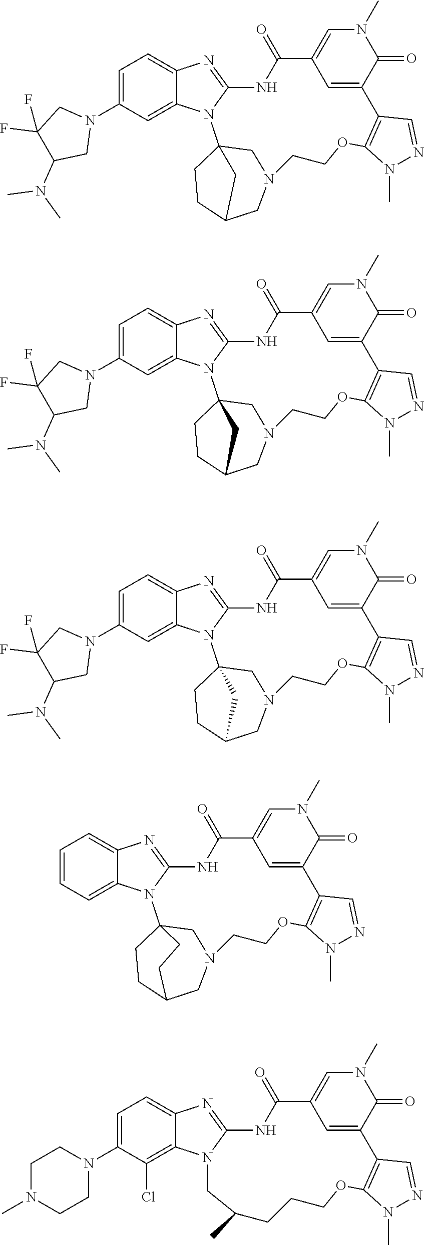 [SMILES: CN(C)C1CN(c2ccc3nc4n(c3c2)C23CCC(CN(CCOc5c(cnn5C)-c5cc(cn(C)c5=O)C(=O)N4)C2)C3)CC1(F)F ; CN(C)C1CN(c2ccc3nc4n(c3c2)[C@@]23CC[C@@H](CN(CCOc5c(cnn5C)-c5cc(cn(C)c5=O)C(=O)N4)C2)C3)CC1(F)F ; CN(C)C1CN(c2ccc3nc4n(c3c2)[C@]23CC[C@H](CN(CCOc5c(cnn5C)-c5cc(cn(C)c5=O)C(=O)N4)C2)C3)CC1(F)F ; Cn1ncc2c1OCCN1CC3CCC(CC3)(C1)n1c(nc3ccccc31)NC(=O)c1cc-2c(=O)n(C)c1 ; C[C@@H]1CCCOc2c(cnn2C)-c2cc(cn(C)c2=O)C(=O)Nc2nc3ccc(N4CCN(C)CC4)c(Cl)c3n2C1]

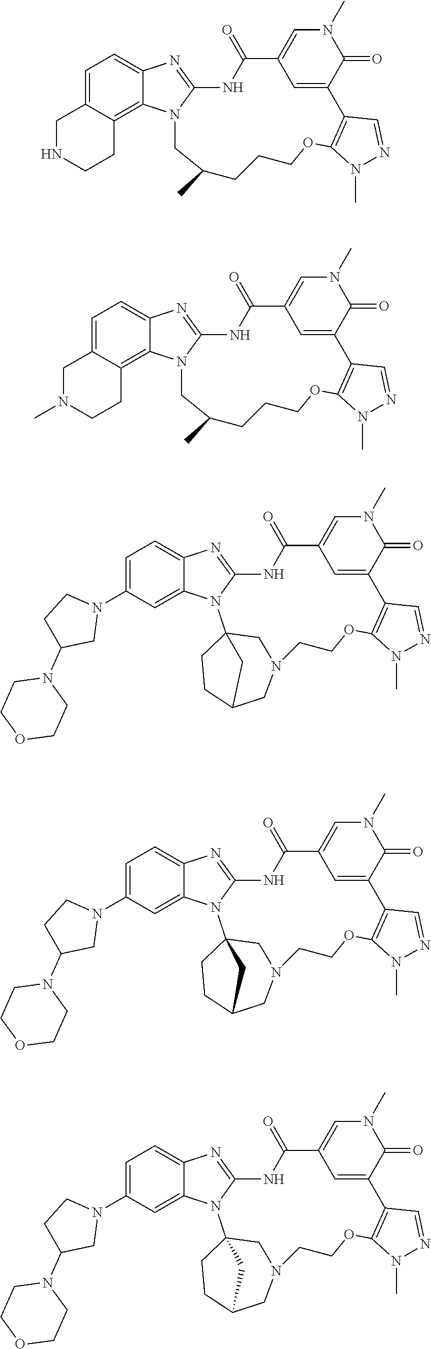

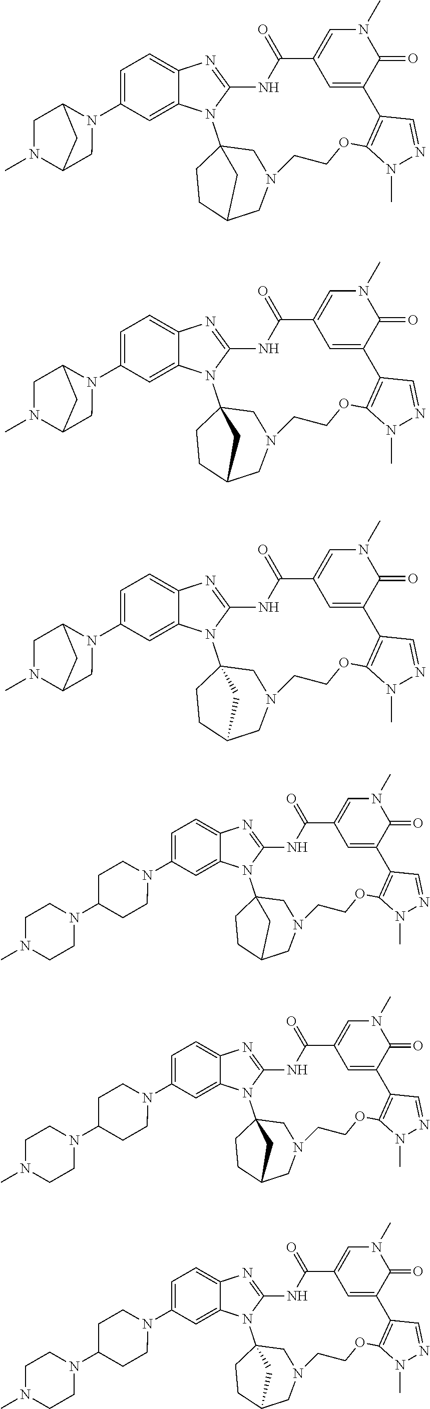

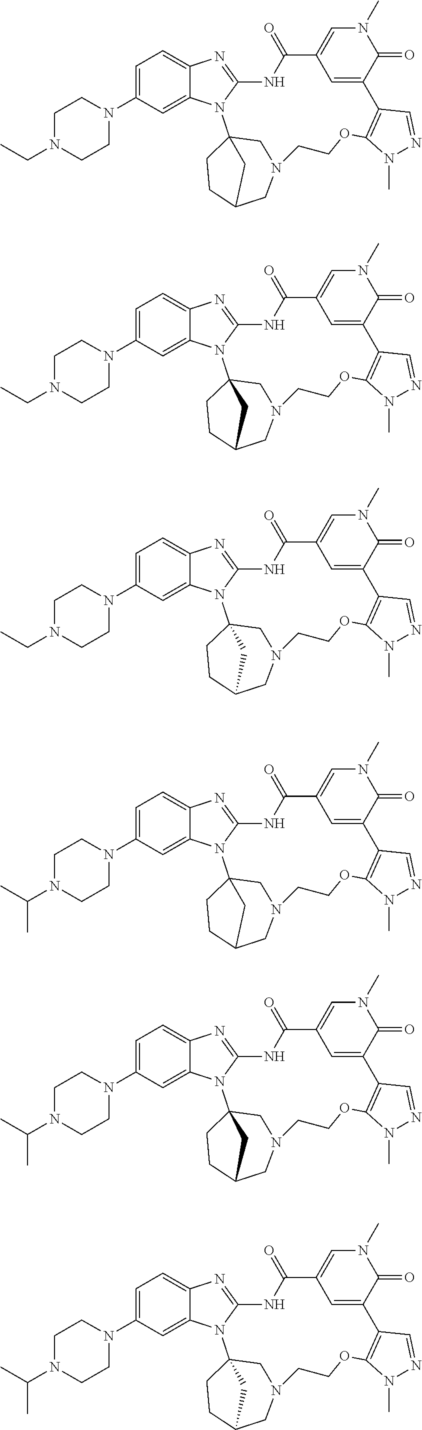

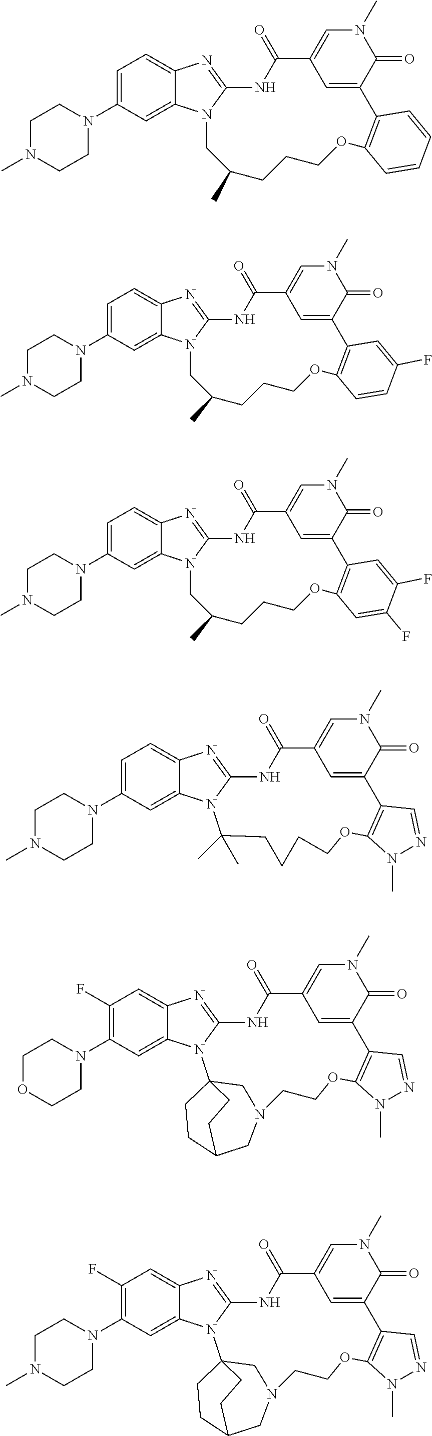 [SMILES: C[C@@H]1CCCOc2ccccc2-c2cc(cn(C)c2=O)C(=O)Nc2nc3ccc(N4CCN(C)CC4)cc3n2C1 ; C[C@@H]1CCCOc2ccc(F)cc2-c2cc(cn(C)c2=O)C(=O)Nc2nc3ccc(N4CCN(C)CC4)cc3n2C1 ; C[C@@H]1CCCOc2cc(F)c(F)cc2-c2cc(cn(C)c2=O)C(=O)Nc2nc3ccc(N4CCN(C)CC4)cc3n2C1 ; CN1CCN(c2ccc3nc4n(c3c2)C(C)(C)CCCCOc2c(cnn2C)-c2cc(cn(C)c2=O)C(=O)N4)CC1 ; Cn1ncc2c1OCCN1CC3CCC(CC3)(C1)n1c(nc3cc(F)c(N4CCOCC4)cc31)NC(=O)c1cc-2c(=O)n(C)c1 ; CN1CCN(c2cc3c(cc2F)nc2n3C34CCC(CC3)CN(CCOc3c(cnn3C)-c3cc(cn(C)c3=O)C(=O)N2)C4)CC1]

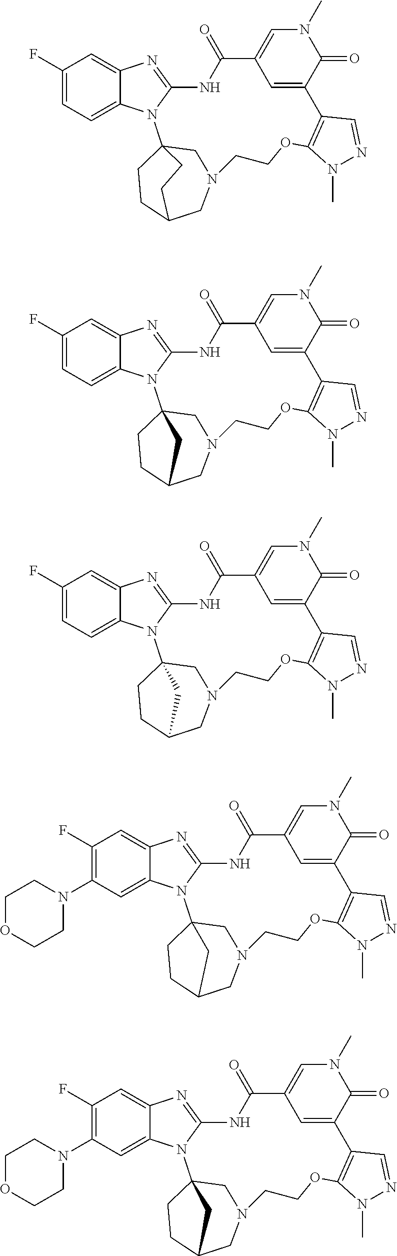 [SMILES: Cn1ncc2c1OCCN1CC3CCC(CC3)(C1)n1c(nc3cc(F)ccc31)NC(=O)c1cc-2c(=O)n(C)c1 ; Cn1ncc2c1OCCN1C[C@@H]3CC[C@@](C3)(C1)n1c(nc3cc(F)ccc31)NC(=O)c1cc-2c(=O)n(C)c1 ; Cn1ncc2c1OCCN1C[C@H]3CC[C@](C3)(C1)n1c(nc3cc(F)ccc31)NC(=O)c1cc-2c(=O)n(C)c1 ; Cn1ncc2c1OCCN1CC3CCC(C3)(C1)n1c(nc3cc(F)c(N4CCOCC4)cc31)NC(=O)c1cc-2c(=O)n(C)c1 ; Cn1ncc2c1OCCN1C[C@@H]3CC[C@@](C3)(C1)n1c(nc3cc(F)c(N4CCOCC4)cc31)NC(=O)c1cc-2c(=O)n(C)c1]

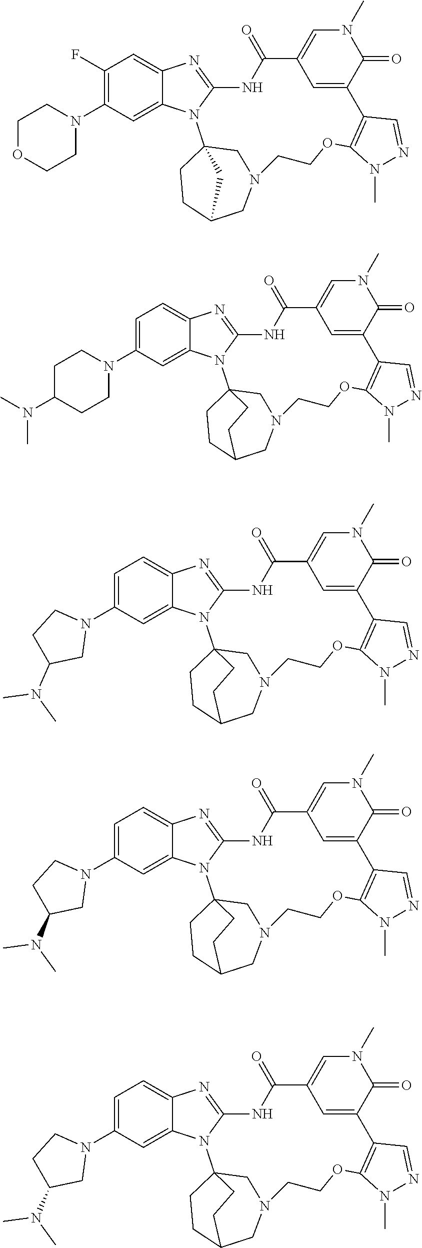 [SMILES: Cn1ncc2c1OCCN1C[C@H]3CC[C@](C3)(C1)n1c(nc3cc(F)c(N4CCOCC4)cc31)NC(=O)c1cc-2c(=O)n(C)c1 ; CN(C)C1CCN(c2ccc3nc4n(c3c2)C23CCC(CC2)CN(CCOc2c(cnn2C)-c2cc(cn(C)c2=O)C(=O)N4)C3)CC1 ; CN(C)C1CCN(c2ccc3nc4n(c3c2)C23CCC(CC2)CN(CCOc2c(cnn2C)-c2cc(cn(C)c2=O)C(=O)N4)C3)C1 ; CN(C)[C@H]1CCN(c2ccc3nc4n(c3c2)C23CCC(CC2)CN(CCOc2c(cnn2C)-c2cc(cn(C)c2=O)C(=O)N4)C3)C1 ; CN(C)[C@@H]1CCN(c2ccc3nc4n(c3c2)C23CCC(CC2)CN(CCOc2c(cnn2C)-c2cc(cn(C)c2=O)C(=O)N4)C3)C1]

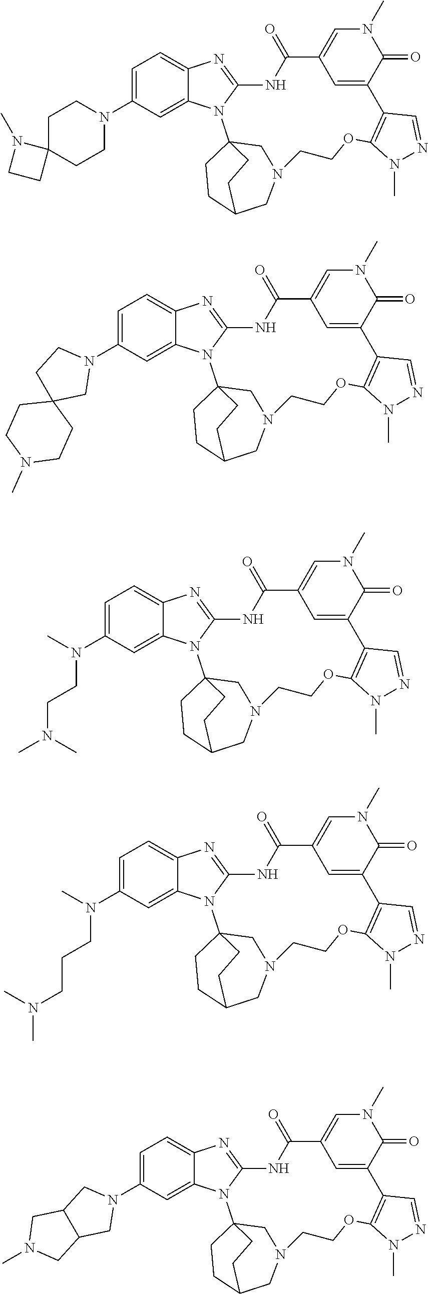 [SMILES: CN1CCC12CCN(c1ccc3nc4n(c3c1)C13CCC(CC1)CN(CCOc1c(cnn1C)-c1cc(cn(C)c1=O)C(=O)N4)C3)CC2 ; CN1CCC2(CC1)CCN(c1ccc3nc4n(c3c1)C13CCC(CC1)CN(CCOc1c(cnn1C)-c1cc(cn(C)c1=O)C(=O)N4)C3)C2 ; CN(C)CCN(C)c1ccc2nc3n(c2c1)C12CCC(CC1)CN(CCOc1c(cnn1C)-c1cc(cn(C)c1=O)C(=O)N3)C2 ; CN(C)CCCN(C)c1ccc2nc3n(c2c1)C12CCC(CC1)CN(CCOc1c(cnn1C)-c1cc(cn(C)c1=O)C(=O)N3)C2 ; CN1CC2CN(c3ccc4nc5n(c4c3)C34CCC(CC3)CN(CCOc3c(cnn3C)-c3cc(cn(C)c3=O)C(=O)N5)C4)CC2C1]

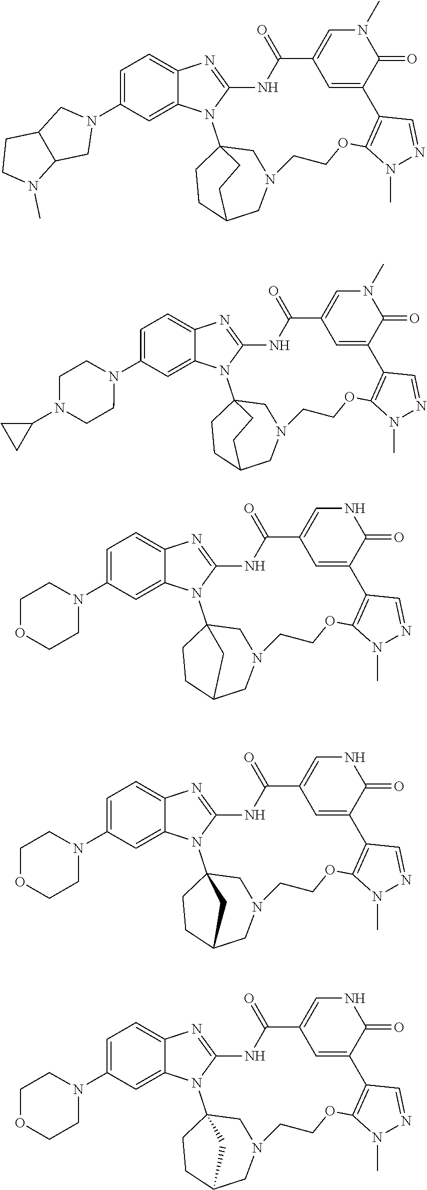

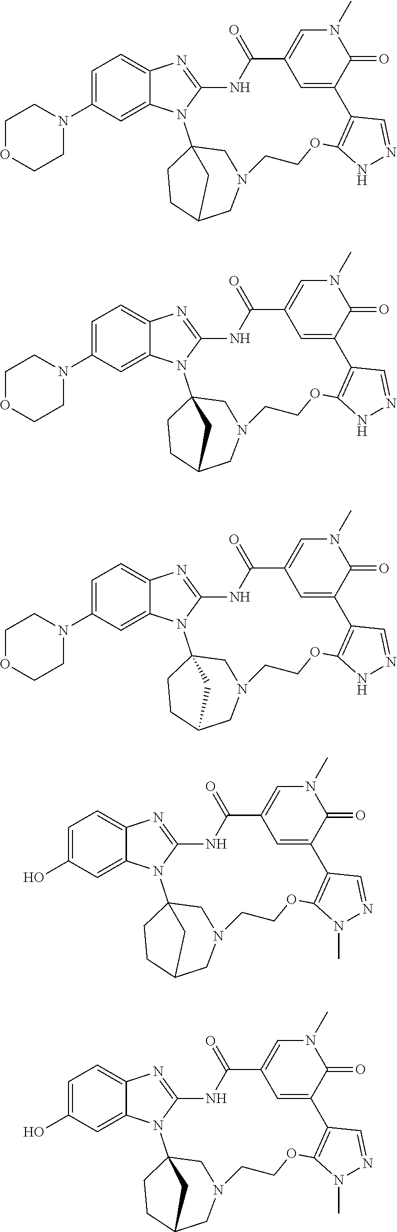 [SMILES: Cn1cc2cc(c1=O)-c1cn[nH]c1OCCN1CC3CCC(C3)(C1)n1c(nc3ccc(N4CCOCC4)cc31)NC2=O ; Cn1cc2cc(c1=O)-c1cn[nH]c1OCCN1C[C@@H]3CC[C@@](C3)(C1)n1c(nc3ccc(N4CCOCC4)cc31)NC2=O ; Cn1cc2cc(c1=O)-c1cn[nH]c1OCCN1C[C@H]3CC[C@](C3)(C1)n1c(nc3ccc(N4CCOCC4)cc31)NC2=O ; Cn1ncc2c1OCCN1CC3CCC(C3)(C1)n1c(nc3ccc(O)cc31)NC(=O)c1cc-2c(=O)n(C)c1 ; Cn1ncc2c1OCCN1C[C@@H]3CC[C@@](C3)(C1)n1c(nc3ccc(O)cc31)NC(=O)c1cc-2c(=O)n(C)c1]

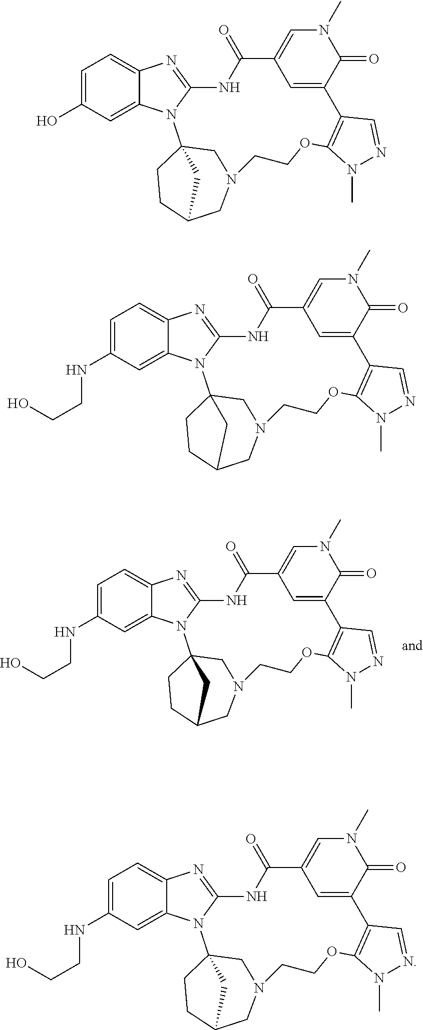 [SMILES: Cn1ncc2c1OCCN1C[C@H]3CC[C@](C3)(C1)n1c(nc3ccc(O)cc31)NC(=O)c1cc-2c(=O)n(C)c1 ; Cn1ncc2c1OCCN1CC3CCC(C3)(C1)n1c(nc3ccc(NCCO)cc31)NC(=O)c1cc-2c(=O)n(C)c1 ; Cn1ncc2c1OCCN1C[C@@H]3CC[C@@](C3)(C1)n1c(nc3ccc(NCCO)cc31)NC(=O)c1cc-2c(=O)n(C)c1 ; Cn1ncc2c1OCCN1C[C@H]3CC[C@](C3)(C1)n1c(nc3ccc(NCCO)cc31)NC(=O)c1cc-2c(=O)n(C)c1]

In one aspect, the present disclosure provides a compound of Formula (II)

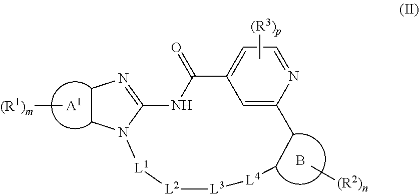

or a pharmaceutically acceptable salt thereof, wherein:
Ring A¹ is 7-12 membered cycloalkyl, 7-12 membered heterocyclyl, 7-12 membered aryl or 7-12 membered heteroaryl;
Ring B is selected from the group consisting of cycloalkyl, heterocyclyl, aryl, and heteroaryl;
L¹ is selected from the group consisting of a bond, alkyl, alkenyl, alkynyl, heteroalkyl, heteroalkenyl and heteroalkynyl, wherein the alkyl, alkenyl, alkynyl, heteroalkyl, heteroalkenyl and heteroalkynyl are optionally substituted with one or more groups independently selected from hydroxyl, halogen, cyano and amino;
L² is selected from the group consisting of a bond, N(R^{A}), alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more R^{B};
wherein R^{A} is selected from a group consisting of hydrogen, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, amino, alkyl and haloalkyl;
wherein each R^{B} is independently selected from a group consisting of hydroxyl, halogen, cyano, amino, alkyl, alkenyl, alkynyl, heteroalkyl, heteroalkenyl, cycloalkyl, heterocyclyl, aryl and heteroaryl;
L³ is selected from the group consisting of a bond, alkyl, alkenyl, alkynyl, heteroalkyl, heteroalkenyl and heteroalkynyl, wherein the alkyl, alkenyl, alkynyl, heteroalkyl, heteroalkenyl and heteroalkynyl are optionally substituted with one or more groups independently selected from hydroxyl, halogen, cyano and amino;
L⁴ is selected from O, S, or N(R^{C});
wherein R^{C} is selected from a group consisting of hydrogen, alkyl, alkenyl, alkynyl, haloalkyl, heteroalkyl, heteroalkenyl, heteroalkynyl, cycloalkyl, heterocyclyl, aryl, and heteroaryl;
each R¹ is independently selected from the group consisting of hydroxy, halogen, cyano, amino, —N(R^{D})₂, alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more R^{E};
wherein each R^{D} is independently selected from a group consisting of hydrogen, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, —N(R^{F})₂ or —OR^{G};
wherein each R^{E} is independently selected from a group consisting of hydrogen, hydroxyl, halogen, cyano, amino, —N(R^{F})₂, -alkyl-N(R^{F})₂, —C(O)OR^{G}, alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, amino and alkyl;
wherein each of R^{F} and R^{G} is independently selected from a group consisting of alkyl, alkenyl, alkynyl, heteroalkyl, heteroalkenyl, heteroalkynyl, cycloalkyl, heterocyclyl, aryl and heteroaryl;
each R² is independently selected from the group consisting of hydrogen, hydroxy, halogen, cyano, amino, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, amino, alkyl and haloalkyl;
each R³ is selected from the group consisting of hydrogen, alkyl, alkenyl, alkynyl, heteroalkyl, heteroalkenyl, heteroalkynyl, cycloalkyl and heterocyclyl, wherein the alkyl, alkenyl, alkynyl, heteroalkyl, heteroalkenyl, heteroalkynyl, cycloalkyl and heterocyclyl are optionally substituted with one or more groups independently selected from hydroxyl, halogen, cyano, amino, alkyl and haloalkyl;
m is an integer from 0 to 5;
n is an integer from 0 to 4; and
p is an integer from 0 to 3.

In some embodiments, Ring A¹ is 7-12 membered heteroaryl.

In certain embodiments, Ring A¹ is tetrahydroisoquinolinyl.

In certain embodiments, Ring A¹ is

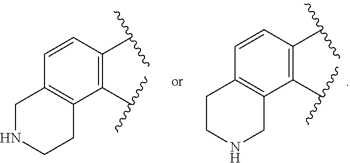 [SMILES: *c1ccc2c(c1*)CCNC2 ; *c1ccc2c(c1*)CNCC2]

In some embodiments, Ring B is aryl. In certain embodiments, Ring B is C₆₋₁₂ aryl, C₆₋₁₁ aryl, C₆₋₁₀ aryl, C₆₋₉ aryl, or C₆₋₈ aryl. In certain embodiments, Ring B is phenyl.

In some embodiments, Ring B is heteroaryl. In certain embodiments, Ring B is 5-12 membered heteroaryl, 5-11 membered heteroaryl, 5-10 membered heteroaryl, 5-9 membered heteroaryl, 5-8 membered heteroaryl, 5-7 membered heteroaryl, or 5-12 membered heteroaryl. In certain embodiments, Ring B is pyridinyl or pyrazolyl.

In certain embodiments, Ring B is selected from a group consisting of

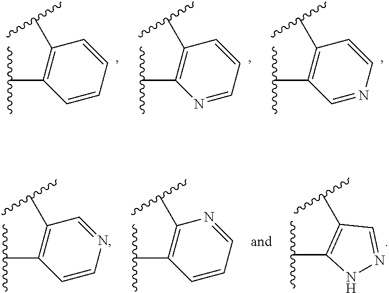

In some embodiments, L¹ is alkyl. In certain embodiments, L¹ is C₁₋₆ alkyl, C₁₋₅ alkyl, C₁₋₄ alkyl, C₁₋₃ alkyl or C₁₋₂ alkyl.

In certain embodiments, wherein L¹ is

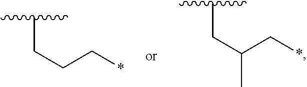

wherein * end of L¹ is connected to L².

In some embodiments, L² is a bond.

In some embodiments, L¹ is alkyl and L² is a bond.

In some embodiments, L³ is alkyl. In certain embodiments, L³ is C₁₋₆ alkyl, C₁₋₅ alkyl, C₁₋₄ alkyl, C₁₋₃ alkyl or C₁₋₂ alkyl. In certain embodiments, L³ is ethyl.

In some embodiments, L⁴ is O or NH.

In some embodiments, -L¹-L²-L³-L⁴- is -alkyl-O—. In certain embodiments, -L¹-L²-L³-L⁴- is —(C₃₋₈ alkyl)-O—. In certain embodiments, -L¹-L²-L³-L⁴- is —(CH₂)₅—O—, —CH₂CH(CH₃)(CH₂)₃—O— or —CH₂CH(CH₂CH₃)(CH₂)₃—O—.

In some embodiment, m is 0.

In some embodiments, m is 1 and R¹ is alkyl optionally substituted with one or more R^{E}. In certain embodiments, m is 1 and R¹ is C₁₋₆ alkyl, C₁₋₅ alkyl, C₁₋₄ alkyl, C₁₋₃ alkyl or C₁₋₂ alkyl optionally substituted with one or more R^{E}. In certain embodiments, R¹ is ethyl optionally substituted with one or more R^{E}.

In some embodiments, R^{E} is —N(R^{F})₂, and R^{F} is alkyl. In certain embodiments, R^{E} is —N(R^{F})₂, and R^{F} is C₁₋₆ alkyl, C₁₋₅ alkyl, C₁₋₄ alkyl, C₁₋₃ alkyl or C₁₋₂ alkyl. In certain embodiments, R^{E} is —N(CH₃)₂.

In some embodiments, m is 1 and R¹ is heterocyclyl optionally substituted with one or more R^{E}. In certain embodiments, m is 1 and R¹ is 3- to 10-membered heterocyclyl, 3- to 9-membered heterocyclyl, 3- to 8-membered heterocyclyl, 3- to 7-membered heterocyclyl, 3- to 6-membered heterocyclyl, or 3- to 5-membered heterocyclyl, each optionally substituted with one or more R^{E}. In certain embodiments, R¹ is oxetanyl. In certain embodiments, R¹ is

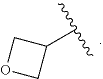 [SMILES: *C1COC1]

In some embodiments, n is 1.

In some embodiments, R² is alkyl. In certain embodiments, R² is C₁₋₆ alkyl, C₁₋₅ alkyl, C₁₋₄ alkyl, C₁₋₃ alkyl or C₁₋₂ alkyl. In certain embodiments, R² is methyl.

In some embodiments, n is 1 and R² is alkyl. In certain embodiments, n is 1 and R² is C₁₋₆ alkyl, C₁₋₅ alkyl, C₁₋₄ alkyl, C₁₋₃ alkyl or C₁₋₂ alkyl. In certain embodiments, n is 1 and R² is methyl.

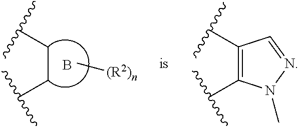

In some embodiments, is

In some embodiments, p is 1.

In some embodiments, R³ is alkyl. In certain embodiments, R³ is C₁₋₆ alkyl, C₁₋₅ alkyl, C₁₋₄ alkyl, C₁₋₃ alkyl or C₁₋₂ alkyl. In certain embodiments, R³ is methyl.

In some embodiments, p is 1 and R³ is alkyl. In certain embodiments, p is 1 and R³ is C₁₋₆ alkyl, C₁₋₅ alkyl, C₁₋₄ alkyl, C₁₋₃ alkyl or C₁₋₂ alkyl. In certain embodiments, p is 1 and R³ is methyl.

In some embodiments,

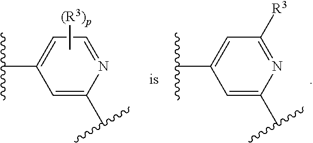

In certain embodiments,

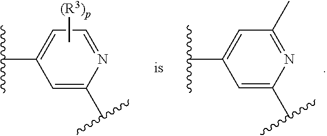

In some embodiments, n is 0 or 1.

In some embodiments, n is 1.

In some embodiments, p is 1.

In another aspect, the present disclosure provides a compound of Formula (III):

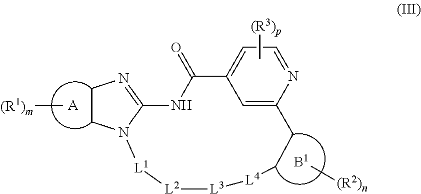

or a pharmaceutically acceptable salt thereof, wherein:
Ring A is selected from the group consisting of cycloalkyl, heterocyclyl, aryl and heteroaryl;
Ring B¹ is 7-12 membered cycloalkyl, 7-12 membered heterocyclyl, 7-12 membered aryl or 7-12 membered heteroaryl;
L¹ is selected from the group consisting of a bond, alkyl, alkenyl, alkynyl, heteroalkyl, heteroalkenyl and heteroalkynyl, wherein the alkyl, alkenyl, alkynyl, heteroalkyl, heteroalkenyl and heteroalkynyl are optionally substituted with one or more groups independently selected from hydroxyl, halogen, cyano and amino;
L² is selected from the group consisting of a bond, N(R^{A}), alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more R^{B};
wherein R^{A} is selected from a group consisting of hydrogen, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, amino, alkyl and haloalkyl;
wherein each R^{B} is independently selected from a group consisting of hydroxyl, halogen, cyano, amino, alkyl, alkenyl, alkynyl, heteroalkyl, heteroalkenyl, cycloalkyl, heterocyclyl, aryl and heteroaryl;
L³ is selected from the group consisting of a bond, alkyl, alkenyl, alkynyl, heteroalkyl, heteroalkenyl and heteroalkynyl, wherein the alkyl, alkenyl, alkynyl, heteroalkyl, heteroalkenyl and heteroalkynyl are optionally substituted with one or more groups independently selected from hydroxyl, halogen, cyano and amino;
L⁴ is selected from O, S, or N(R^{C});
wherein R^{C} is selected from a group consisting of hydrogen, alkyl, alkenyl, alkynyl, haloalkyl, heteroalkyl, heteroalkenyl, heteroalkynyl, cycloalkyl, heterocyclyl, aryl, and heteroaryl;
each R¹ is independently selected from the group consisting of hydroxy, halogen, cyano, amino, —N(R^{D})₂, alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more R^{E};
wherein each R^{D} is independently selected from a group consisting of hydrogen, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, —N(R^{F})₂ or —OR^{G};
wherein each R^{E} is independently selected from a group consisting of hydrogen, hydroxyl, halogen, cyano, amino, —N(R^{F})₂, -alkyl-N(R^{F})₂, —C(O)OR^{G}, alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, amino and alkyl;
wherein each of R^{F} and R^{G} is independently selected from a group consisting of alkyl, alkenyl, alkynyl, heteroalkyl, heteroalkenyl, heteroalkynyl, cycloalkyl, heterocyclyl, aryl and heteroaryl;
each R² is independently selected from the group consisting of hydrogen, hydroxy, halogen, cyano, amino, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, amino, alkyl and haloalkyl;
each R³ is selected from the group consisting of hydrogen, halogen, alkyl, alkenyl, alkynyl, heteroalkyl, heteroalkenyl, heteroalkynyl, cycloalkyl and heterocyclyl, wherein the alkyl, alkenyl, alkynyl, heteroalkyl, heteroalkenyl, heteroalkynyl, cycloalkyl and heterocyclyl are optionally substituted with one or more groups independently selected from hydroxyl, halogen, cyano, amino, alkyl and haloalkyl;
m is an integer from 0 to 5;
n is an integer from 0 to 4; and
p is an integer from 0 to 3.

In some embodiments, Ring A is aryl. In certain embodiments, Ring A is C₆₋₁₂ aryl, C₆₋₁₁ aryl, C₆₋₁₀ aryl, C₆₋₉ aryl, or C₆₋₈ aryl. In certain embodiments, Ring A is phenyl.

In some embodiments, Ring A is heteroaryl. In certain embodiments, Ring A is 5-12 membered heteroaryl, 5-11 membered heteroaryl, 5-10 membered heteroaryl, 5-9 membered heteroaryl, 5-8 membered heteroaryl, 5-7 membered heteroaryl, or 5-6 membered heteroaryl. In certain embodiments, Ring A is selected from a group consisting of furanyl, thiophenyl, pyrrolyl, pyridinyl, pyranyl, pyrimidinyl, pyridazinyl, pyrazinyl and tetrahydroisoquinolinyl.

In some embodiments, Ring B¹ is 7-12 membered heteroaryl.

In certain embodiments, Ring B¹ is pyrazolopyridinyl.

In certain embodiments, Ring B1 is

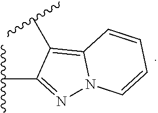 [SMILES: *c1nn2ccccc2c1*]

In some embodiments, L¹ is a bond.

In some embodiments, L¹ is alkyl. In certain embodiments, L¹ is C₁₋₆ alkyl, C₁₋₅ alkyl, C₁₋₄ alkyl, C₁₋₃ alkyl or C₁₋₂ alkyl.

In certain embodiments, L¹ is selected from a group consisting of

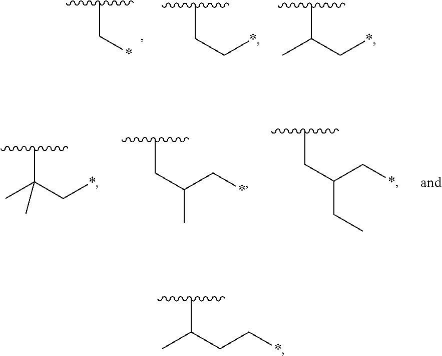

wherein * end of L¹ is connected to L².

In some embodiments, L² is a bond.

In some embodiments, L² is N(R^{A}), and R^{A} is selected from alkyl or heterocyclyl, wherein the alkyl or heterocyclyl are are optionally substituted with one or more halogen or alkyl. In certain embodiments, L² is N(R^{A}), and R^{A} is ethyl, difluoroethyl, trifluoroethyl or oxetanyl.

In some embodiments, L² is cycloalkyl optionally substituted with one or more R^{B}. In certain embodiments, L² is C₃₋₁₀ cycloalkyl, C₃₋₉ cycloalkyl, C₃₋₈ cycloalkyl, C3.7 cycloalkyl, C₃₋₆ cycloalkyl, or C₃₋₅ cycloalkyl, each optionally substituted with one or more R^{B}.

In certain embodiments, L² is

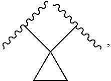 [SMILES: *C1(*)CC1]

which is optionally substituted with one or more R^{B}.

In some embodiments, L² is heterocyclyl optionally substituted with one or more R^{B}.

In certain embodiments, L² is heterocyclyl containing one or more heteroatoms selected from N, O or S. In certain embodiments, L² is 3-12 membered heterocyclyl, 3-11 membered heterocyclyl, 3-10 membered heterocyclyl, or 3-9 membered heterocyclyl, each optionally substituted with one or more R^{B}.

In certain embodiments, the L² is heterocyclyl selected from a group consisting of

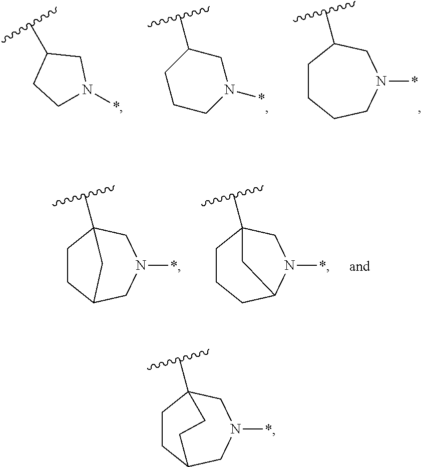

each of which is optionally substituted with one or more R^{B}, wherein * end of L² is connected to L³.

In some embodiments, L² is cycloalkyl or heterocyclyl substituted with one or more R^{B}, and R^{B} is alkyl. In certain embodiments, R^{B} is C₁₋₆ alkyl, C₁₋₅ alkyl, C₁₋₄ alkyl, C₁₋₃ alkyl or C₁₋₂ alkyl. In certain embodiments, R^{B} is methyl.

In some embodiments, L² is selected from a group consisting of:

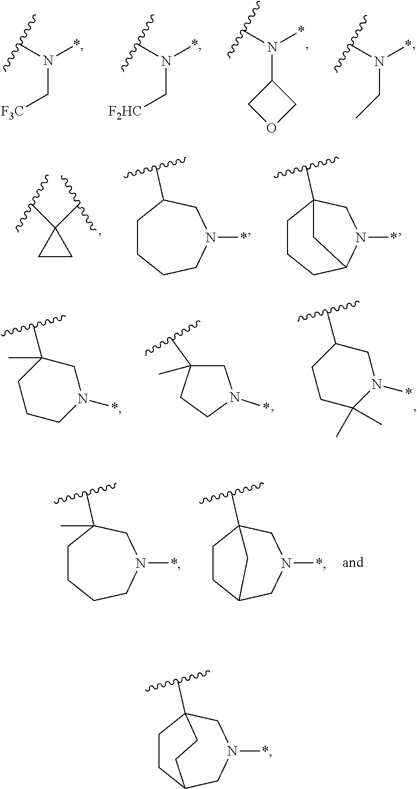

wherein * end of L² is connected to L³.

In some embodiments, L³ is alkyl. In some embodiments, L³ is C₁₋₆ alkyl, C₁₋₅ alkyl, C₁₋₄ alkyl, C₁₋₃ alkyl or C₁₋₂ alkyl.

In certain embodiments, L³ is ethyl.

In some embodiments, L⁴ is O or NH.

In some embodiments, L¹ is a bond and L² is a bond or heterocyclyl optionally substituted with one or more R^{B}.

In some embodiments, L¹ is alkyl and L² is a bond, N(R^{A}), or cycloalkyl optionally substituted with one or more R^{B}.

In some embodiments, -L¹-L²-L³-L⁴- is -alkyl-O—. In certain embodiments, -L¹-L²-L³-L⁴- is —(C₃₋₈ alkyl)-O—. In certain embodiments, -L¹-L²-L³-L⁴- is —(CH₂)₅—O—, —CH₂CH(CH₃)(CH2)₃—O— or —CH₂CH(CH₂CH₃)(CH₂)₃—O—.

In some embodiments, -L¹-L²-L³-L⁴- is -heterocyclyl-alkyl-O—, wherein the heterocyclyl is optionally substituted with one or more R^{B}. In certain embodiments, -L¹-L²-L³-L⁴- is -(5-10 membered heterocyclyl)-(C₁₋₆ alkyl)-O—, wherein the 5-10 membered heterocyclyl is optionally substituted with one or more R^{B}. In certain embodiments -L¹-L²-L³-L⁴- is selected from the group consisting of:

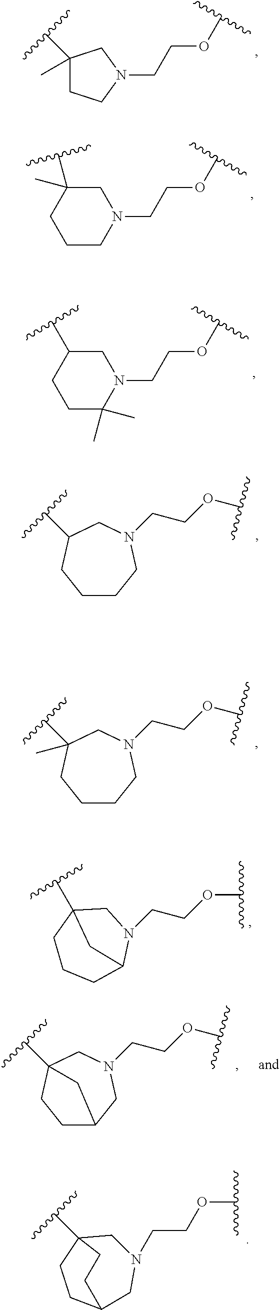

In some embodiments, -L¹-L²-L³-L⁴- is -alkyl-N(R^{A})-alkyl-O—, wherein R^{A} is selected from alkyl or heterocyclyl, wherein the alkyl or heterocyclyl are are optionally substituted with one or more halogen or alkyl. In certain embodiments, -L¹-L²-L³-L⁴- is —(C₁₋₆ alkyl)-N(R^{A})—(C₁₋₆ alkyl)-O—, wherein R^{A} is selected from C₁₋₆ alkyl or 3- to 6-membered heterocyclyl, wherein the alkyl or heterocyclyl are are optionally substituted with one or more halogen or C₁₋₆ alkyl. In certain embodiments, -L¹-L²-L³-L⁴- is

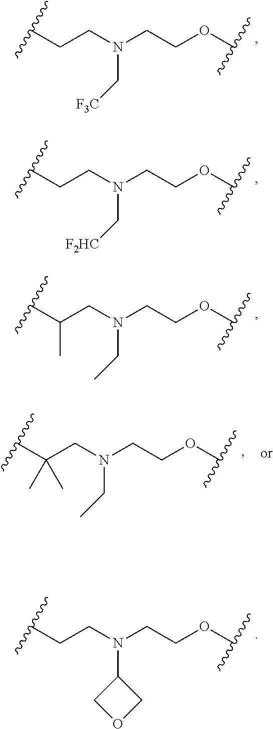 [SMILES: *CCN(CCO*)CC(F)(F)F ; *CCN(CCO*)CC(F)F ; *OCCN(CC)CC(*)C ; *OCCN(CC)CC(*)(C)C ; *CCN(CCO*)C1COC1]

In some embodiments, -L¹-L²-L³-L⁴- is -alkyl-cycloalkyl-alkyl-O—, wherein the cycloalkyl is optionally substituted with one or more R^{B}. In certain embodiments, -L¹-L²-L³-L⁴- is —(C₁₋₆ alkyl)-(C₃₋₆ cycloalkyl)-(C₁₋₆ alkyl)-O—, wherein the C₃₋₆ cycloalkyl is optionally substituted with one or more R^{B}. In certain embodiments, -L¹-L²-L³-L⁴- is

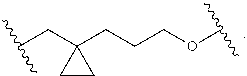 [SMILES: *CC1(CCCO*)CC1]

In some embodiments, R¹ is halogen and m is 1.

In some embodiments, R¹ is —N(R^{D})₂ and m is 1.

In some embodiments, R¹ is —N(R^{D})₂, and each R^{D} is independently alkyl optionally substituted with one or more groups independently selected from —N(R^{F})₂ or —OR^{G}. In certain embodiments, R¹ is —N(R^{D})₂, and each R^{D} is C₁₋₆ alkyl, C₁₋₅ alkyl, C₁₋₄ alkyl, C₁₋₃ alkyl or C₁₋₂ alkyl optionally substituted with one or more groups independently selected from —N(R^{F})₂ or —OR^{G}. In certain embodiments, R¹ is —N(R^{D})₂, and each R^{D} is independently methyl, methoxyethyl, N,N-dimethylaminoethyl, or N,N-dimethylaminopropyl.

In some embodiments, R¹ is heterocyclyl optionally substituted with one or more R^{E} and m is 1. In certain embodiments, R¹ is 3- to 10-membered heterocyclyl optionally substituted with one or more R^{E} and m is 1.

In certain embodiments, R¹ is selected from a group consisting of:

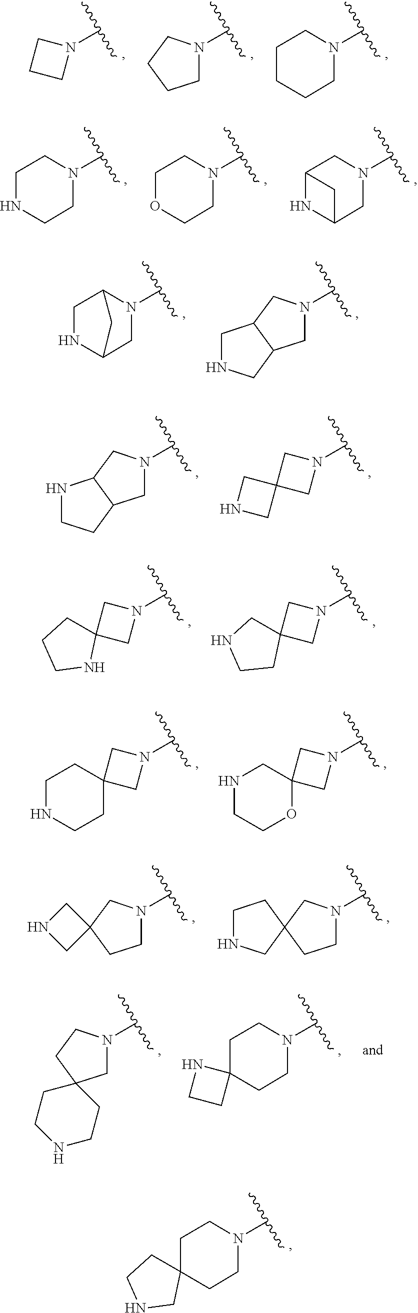

each of which is optionally substituted with one or more R^{E}.

In some embodiments, R^{E} is halogen.

In certain embodiments, R^{E} is F.

In certain embodiments, R^{E} is —N(R^{F})₂, -alkyl-N(R^{F})₂, —C(O)OR^{G}, or alkyl optionally substituted with one or more halogen.

In some embodiments, each of R^{F} and R^{G} is independently alkyl. In certain embodiments, each of R^{F} and R^{G} is independently C₁₋₆ alkyl, C₁₋₅ alkyl, C₁₋₄ alkyl, C₁₋₃ alkyl or C₁₋₂ alkyl.

In certain embodiments, each of R^{F} and R^{G} is independently C₁₋₃ alkyl.

In certain embodiments, R^{E} is selected from a group consisting of —N(CH₃)₂, —C₁₋₃ alkyl-N(CH₃)₂, —C(O)O(tert-butyl), methyl, ethyl, and trifluoroethyl.

In certain embodiments, R^{E} is cycloalkyl or heterocyclyl, wherein the cycloalkyl and heterocyclyl are optionally substituted with one or more alkyl and/or halogen.

In certain embodiments, R^{E} is selected from cyclopropyl, morpholinyl, piperazinyl, oxetanyl or azetidinyl, each of which is optionally substituted with one or more alkyl and/or halogen.

In certain embodiments, R^{E} is selected from cyclopropyl, morpholinyl, piperazinyl, oxetanyl or azetidinyl, each of which is optionally substituted with one or more alkyl and/or halogen.

In certain embodiments, R² is halogen and n is 1 or 2.

In certain embodiments, R² is F.

In some embodiments, R² is alkyl or cycloalkyl, wherein the alkyl and cycloalkyl are optionally substituted with one or more halogen. In certain embodiments, R² is C₁₋₆ alkyl or C₃₋₆ cycloalkyl, wherein the C₁₋₆ alkyl and C₃₋₆ cycloalkyl are optionally substituted with one or more halogen. In certain embodiments, R² is methyl, ethyl, cyclopropyl or trifluoroethyl.

In some embodiments,

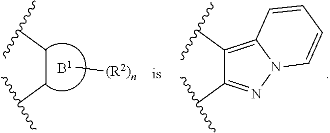

In some embodiments, R³ is alkyl or cycloalkyl. In certain embodiments, R³ is C₁₋₆ alkyl or C₃₋₆ cycloalkyl. In certain embodiments, R³ is methyl, ethyl or cyclopropyl.

In some embodiments, p is 1 and R³ is alkyl. In certain embodiments, p is 1 and R³ is C₁₋₆ alkyl, C₁₋₅ alkyl, C₁₋₄ alkyl, C₁₋₃ alkyl or C₁₋₂ alkyl. In certain embodiments, p is 1 and R³ is methyl.

In some embodiments,

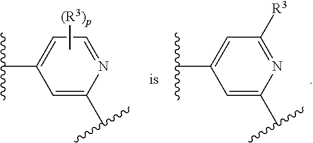

In certain embodiments,

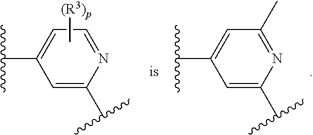

In some embodiments, m is 0, 1 or 2.

In some embodiments, n is 0 or 1.

In some embodiments, p is 1.

In another aspect, the present disclosure provides a compound of Formula (IV):

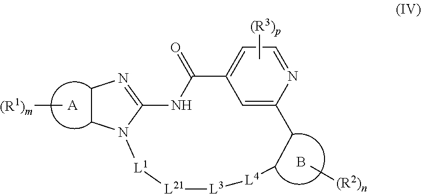

or a pharmaceutically acceptable salt thereof, wherein:
Ring A is selected from the group consisting of cycloalkyl, heterocyclyl, aryl and heteroaryl;
Ring B is selected from the group consisting of cycloalkyl, heterocyclyl, aryl, and heteroaryl;
L¹ is selected from the group consisting of a bond, alkyl, alkenyl, alkynyl, heteroalkyl, heteroalkenyl and heteroalkynyl, wherein the alkyl, alkenyl, alkynyl, heteroalkyl, heteroalkenyl and heteroalkynyl are optionally substituted with one or more groups independently selected from hydroxyl, halogen, cyano and amino;
L²¹ is 7-12 membered cycloalkyl or 7-12 membered heterocyclyl, wherein the cycloalkyl and heterocyclyl are optionally substituted with one or more R^{B};
wherein each R^{B} is independently selected from a group consisting of hydroxyl, halogen, cyano, amino, alkyl, alkenyl, alkynyl, heteroalkyl, heteroalkenyl, cycloalkyl, heterocyclyl, aryl and heteroaryl;
L³ is selected from the group consisting of a bond, alkyl, alkenyl, alkynyl, heteroalkyl, heteroalkenyl and heteroalkynyl, wherein the alkyl, alkenyl, alkynyl, heteroalkyl, heteroalkenyl and heteroalkynyl are optionally substituted with one or more groups independently selected from hydroxyl, halogen, cyano and amino;
L⁴ is selected from O, S, or N(R^{C});
wherein R^{C} is selected from a group consisting of hydrogen, alkyl, alkenyl, alkynyl, haloalkyl, heteroalkyl, heteroalkenyl, heteroalkynyl, cycloalkyl, heterocyclyl, aryl, and heteroaryl;
each R¹ is independently selected from the group consisting of hydroxy, halogen, cyano, amino, —N(R^{D})₂, alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more R^{E};
wherein each R^{D} is independently selected from a group consisting of hydrogen, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, —N(R^{F})₂ or —OR^{G};
wherein each R^{E} is independently selected from a group consisting of hydrogen, hydroxyl, halogen, cyano, amino, —N(R^{F})₂, -alkyl-N(R^{F})₂, —C(O)OR^{G}, alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, amino and alkyl;
wherein each of R^{F} and R^{G} is independently selected from a group consisting of alkyl, alkenyl, alkynyl, heteroalkyl, heteroalkenyl, heteroalkynyl, cycloalkyl, heterocyclyl, aryl and heteroaryl;
each R² is independently selected from the group consisting of hydrogen, hydroxy, halogen, cyano, amino, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, amino, alkyl and haloalkyl;
R³ is selected from the group consisting of hydrogen, alkyl, alkenyl, alkynyl, heteroalkyl, heteroalkenyl, heteroalkynyl, cycloalkyl and heterocyclyl, wherein the alkyl, alkenyl, alkynyl, heteroalkyl, heteroalkenyl, heteroalkynyl, cycloalkyl and heterocyclyl are optionally substituted with one or more groups independently selected from hydroxyl, halogen, cyano, amino, alkyl and haloalkyl;
m is an integer from 0 to 5;
n is an integer from 0 to 4; and
p is an integer from 0 to 3.

In some embodiments, Ring A is aryl. In certain embodiments, Ring A is C₆₋₁₂ aryl, C₆₋₁₁ aryl, C₆₋₁₀ aryl, C₆₋₉ aryl, or C₆₋₈ aryl. In certain embodiments, Ring A is phenyl.

In some embodiments, Ring A is heteroaryl. In certain embodiments, Ring A is 5-12 membered heteroaryl, 5-11 membered heteroaryl, 5-10 membered heteroaryl, 5-9 membered heteroaryl, 5-8 membered heteroaryl, 5-7 membered heteroaryl or 5-6 membered heteroaryl. In certain embodiments, Ring A is selected from a group consisting of furanyl, thiophenyl, pyrrolyl, pyridinyl, pyranyl, pyrimidinyl, pyridazinyl, pyrazinyl and tetrahydroisoquinolinyl.

In some embodiments, Ring B is aryl. In certain embodiments, Ring B is C₆₋₁₂ aryl, C₆₋₁₁ aryl, C₆₋₁₀ aryl, C₆₋₉ aryl, or C₆₋₈ aryl. In certain embodiments, Ring B is phenyl.

In some embodiments, Ring B is heteroaryl. In certain embodiments, Ring B is 5-12 membered heteroaryl, 5-11 membered heteroaryl, 5-10 membered heteroaryl, 5-9 membered heteroaryl, 5-8 membered heteroaryl, 5-7 membered heteroaryl or 5-6 membered heteroaryl.

In certain embodiments, Ring B is pyridinyl or pyrazolyl.

In certain embodiments, Ring B is selected from a group consisting of

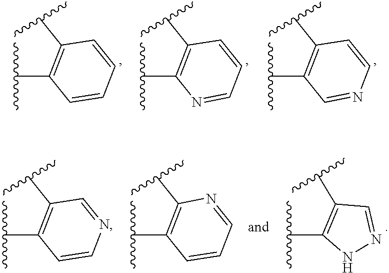

In some embodiments, L¹ is a bond.

In some embodiments, L²¹ is 7-10 membered heterocyclyl containing one or more heteroatoms selected from N or O.

In certain embodiments, L²¹ is selected from a group consisting of

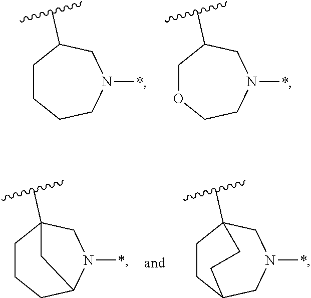

wherein * end of L²¹ is connected to L³.

In some embodiments, L³ is alkyl. In certain embodiments, L³ is C₁₋₆ alkyl, C₁₋₅ alkyl, C₁₋₄ alkyl, C₁₋₃ alkyl or C₁₋₂ alkyl.

In certain embodiments, L³ is ethyl.

In some embodiments, L⁴ is O or NH.

In some embodiments, -L¹-L²¹-L³-L⁴- is -heterocyclyl-alkyl-O—, wherein the heterocyclyl is optionally substituted with one or more R^{B}. In certain embodiments, -L¹-L²¹-L³-L⁴- is -(5-10 membered heterocyclyl)-(C₁₋₆ alkyl)-O—, wherein the 5-10 membered heterocyclyl is optionally substituted with one or more R^{B}.

In certain embodiments, -L¹-L²¹-L³-L⁴- is selected from the group consisting of:

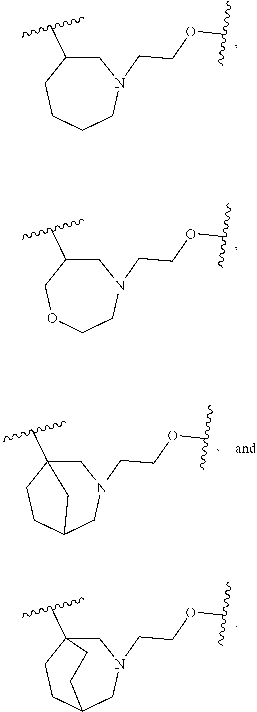 [SMILES: *OCCN1CCCCC(*)C1 ; *OCCN1CCOCC(*)C1 ; *OCCN1CC2CCC(*)(C2)C1 ; *OCCN1CC2CCC(*)(CC2)C1]

In some embodiments, R¹ is halogen and m is 1.

In some embodiments, R¹ is —N(R^{D})₂ and m is 1.

In some embodiments, R¹ is —N(R^{D})₂, and each R^{D} is independently alkyl optionally substituted with one or more groups independently selected from —N(R^{F})₂ or —OR^{G}. In certain embodiments, R¹ is —N(R^{D})₂, and each R^{D} is C₁₋₆ alkyl, C₁₋₅ alkyl, C₁₋₄ alkyl, C₁₋₃ alkyl or C₁₋₂ alkyl optionally substituted with one or more groups independently selected from —N(R^{F})₂ or —OR^{G}. In certain embodiments, R¹ is —N(R^{D})₂, and each R^{D} is independently methyl, methoxyethyl, or N,N-dimethylaminopropyl.

In some embodiments, R¹ is heterocyclyl optionally substituted with one or more R^{E} and m is 1. In certain embodiments, R¹ is 3- to 10-membered heterocyclyl optionally substituted with one or more R^{E} and m is 1.

In certain embodiments, R¹ is selected from a group consisting of:

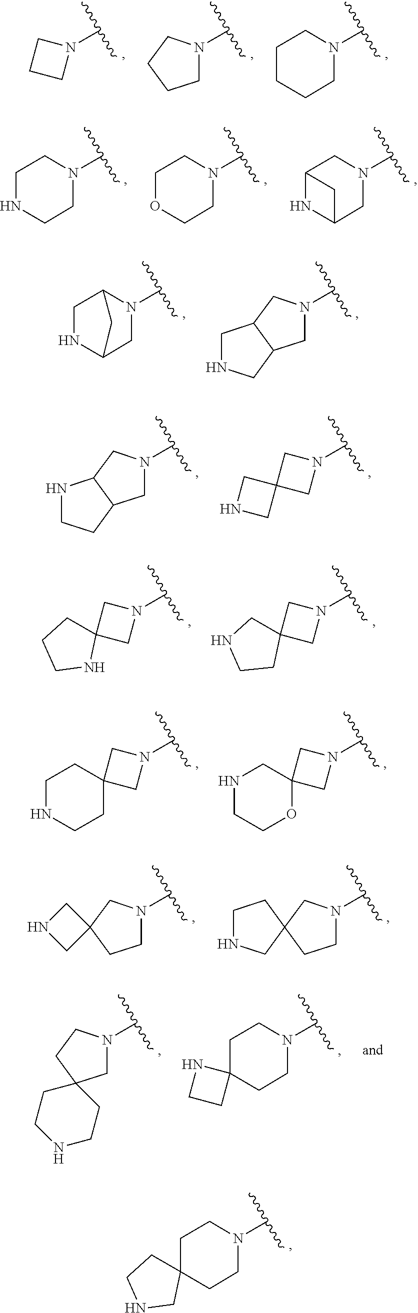

each of which is optionally substituted with one or more R^{E}.

In some embodiments, R^{E} is halogen.

In certain embodiments, R^{E} is F.

In certain embodiments, R^{E} is —N(R^{F})₂, -alkyl-N(R^{F})₂, —C(O)OR^{G}, or alkyl optionally substituted with one or more halogen.

In some embodiments, each of R^{F} and R^{G} is independently alkyl. In certain embodiments, each of R^{F} and R^{G} is independently C₁₋₆ alkyl, C₁₋₅ alkyl, C₁₋₄ alkyl, C₁₋₃ alkyl or C₁₋₂ alkyl.

In certain embodiments, each of R^{F} and R^{G} is independently C₁₋₃ alkyl.

In certain embodiments, R^{E} is selected from a group consisting of —N(CH₃)₂, —C₁₋₃ alkyl-N(CH₃)₂, —C(O)O(tert-butyl), methyl, ethyl, and trifluoroethyl.

In certain embodiments, R^{E} is cycloalkyl or heterocyclyl, wherein the cycloalkyl and heterocyclyl are optionally substituted with one or more alkyl and/or halogen.

In certain embodiments, R^{E} is selected from cyclopropyl, morpholinyl, piperazinyl, oxetanyl or azetidinyl, each of which is optionally substituted with one or more alkyl and/or halogen.

In certain embodiments, R^{E} is selected from cyclopropyl, morpholinyl, piperazinyl, oxetyl or azetidinyl, each of which is optionally substituted with one or more alkyl and/or halogen.

In certain embodiments, R¹ is selected from a group consisting of:

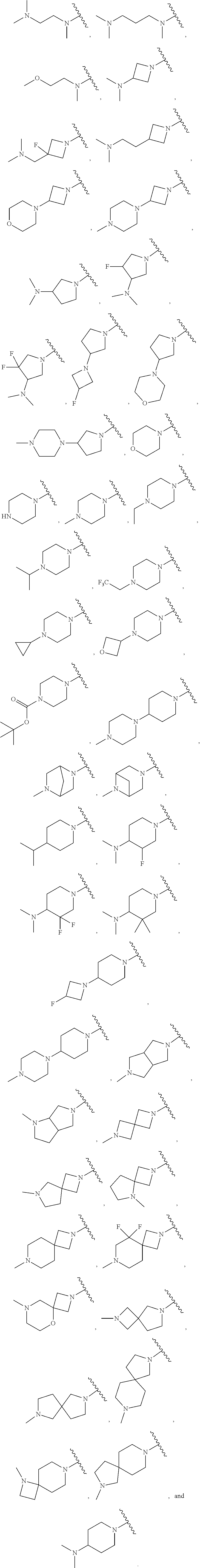

In certain embodiments, R² is halogen and n is 1 or 2.

In certain embodiments, R² is F.

In some embodiments, R² is alkyl or cycloalkyl, wherein the alkyl and cycloalkyl are optionally substituted with one or more halogen. In certain embodiments, R² is C₁₋₆ alkyl or C₃₋₆ cycloalkyl, wherein the C₁₋₆ alkyl and C₃₋₆ cycloalkyl are optionally substituted with one or more halogen. In certain embodiments, R² is methyl, ethyl, cyclopropyl or trifluoroethyl.

In certain embodiments, R² is methyl.

In some embodiments, R³ is alkyl. In certain embodiments, each of R³ is independently C₁₋₆ alkyl, C₁₋₅ alkyl, C₁₋₄ alkyl, C₁₋₃ alkyl or C₁₋₂ alkyl. In certain embodiments, R³ is methyl.

In some embodiments, p is 1 and R³ is alkyl. In certain embodiments, p is 1 and R³ is C₁₋₆ alkyl, C₁₋₅ alkyl, C₁₋₄ alkyl, C₁₋₃ alkyl or C₁₋₂ alkyl. In certain embodiments, p is 1 and R³ is methyl.

In some embodiments,

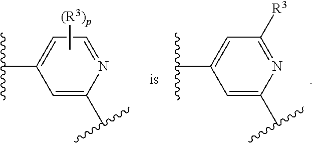

In certain embodiments,

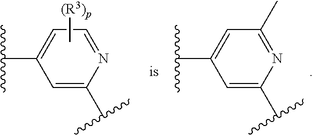

In some embodiments, m is 1.

In some embodiments, n is 1.

In some embodiments, p is 1.

Exemplary compounds of the present disclosure are set forth below.

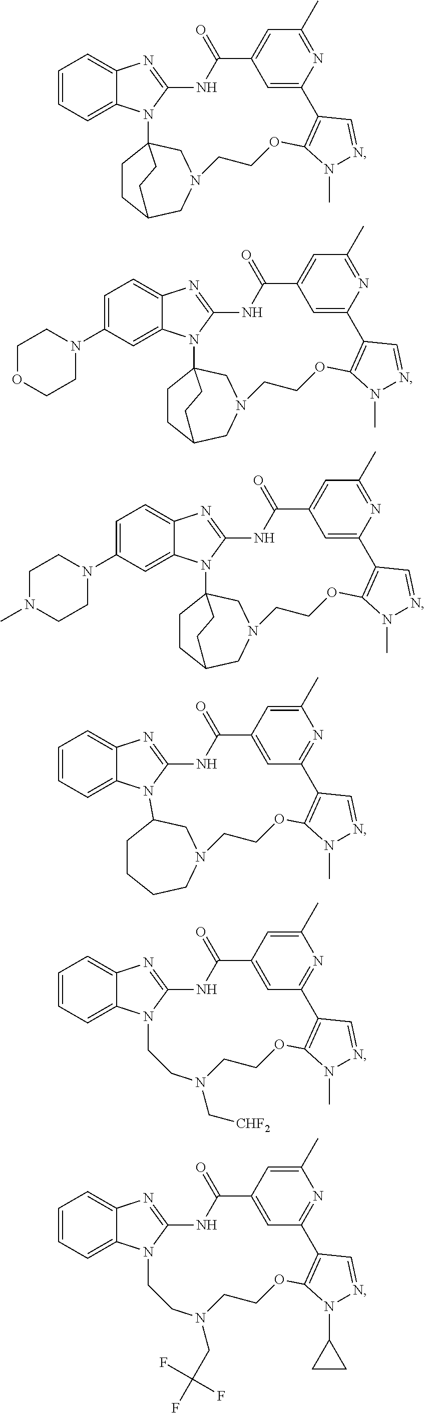

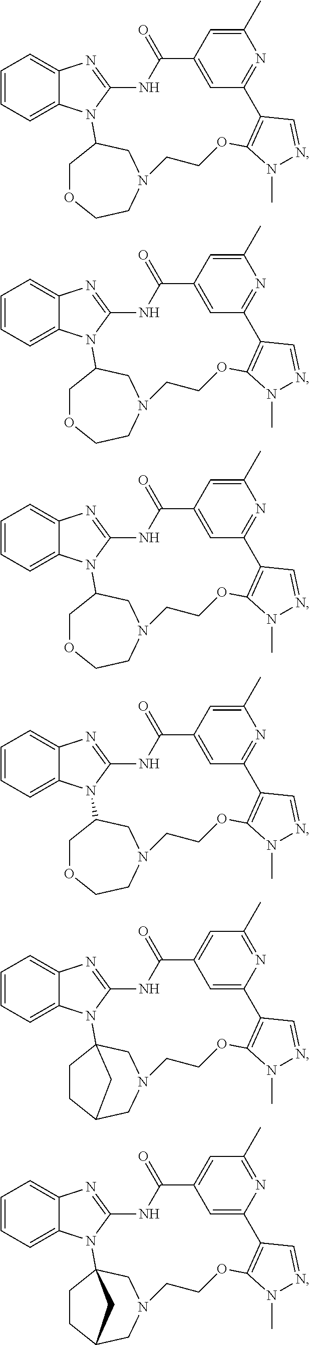

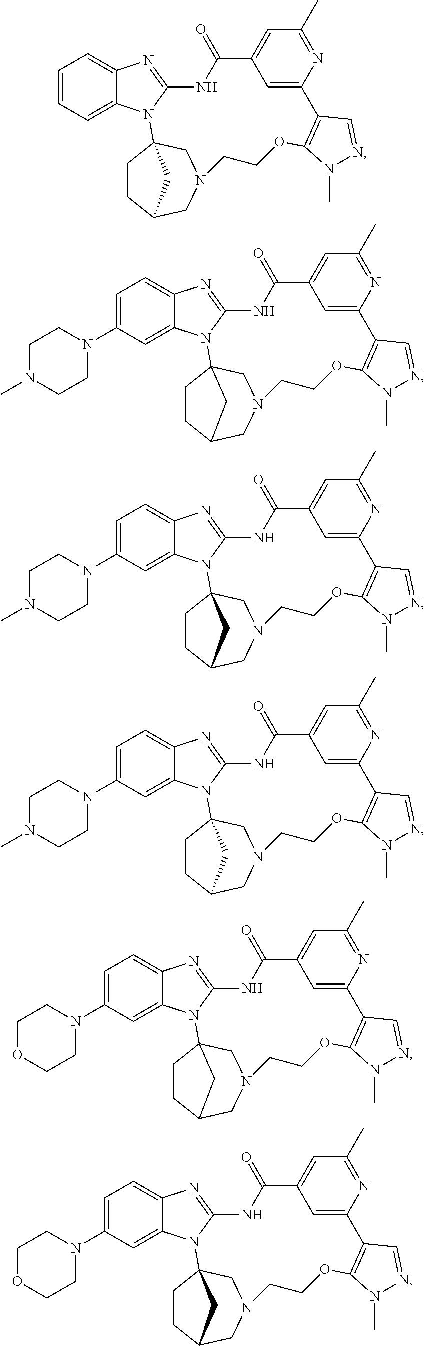

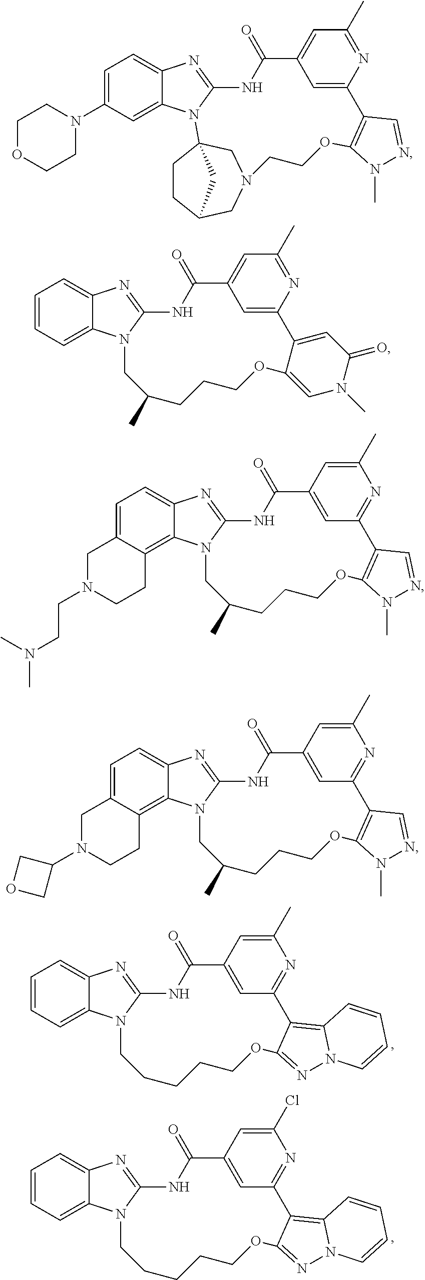

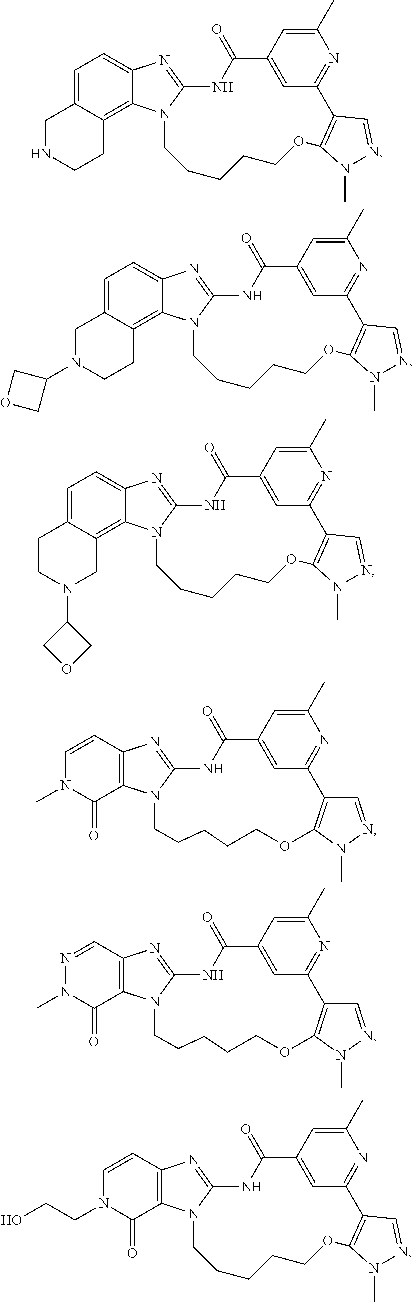

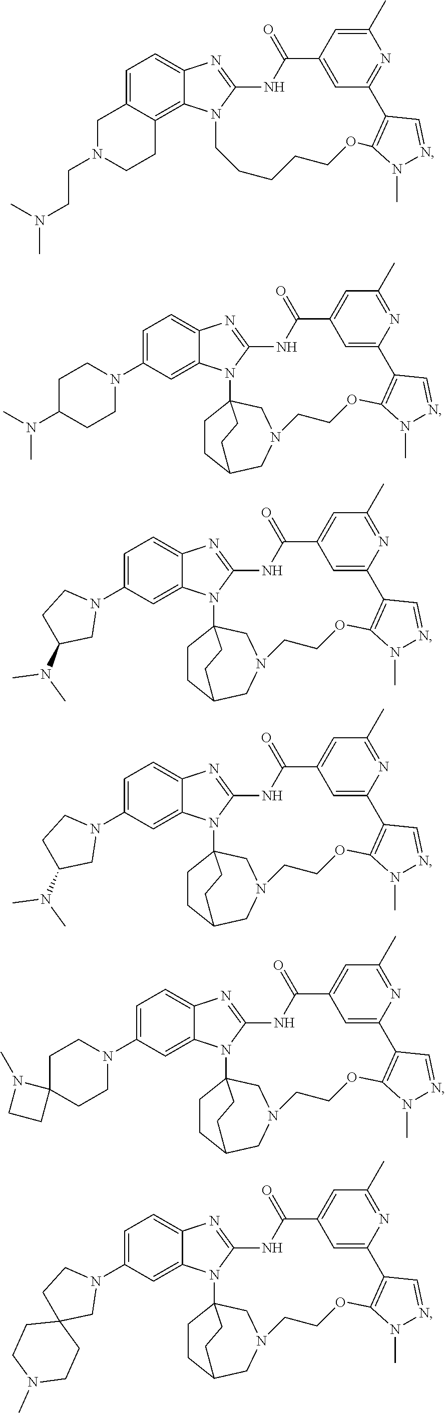

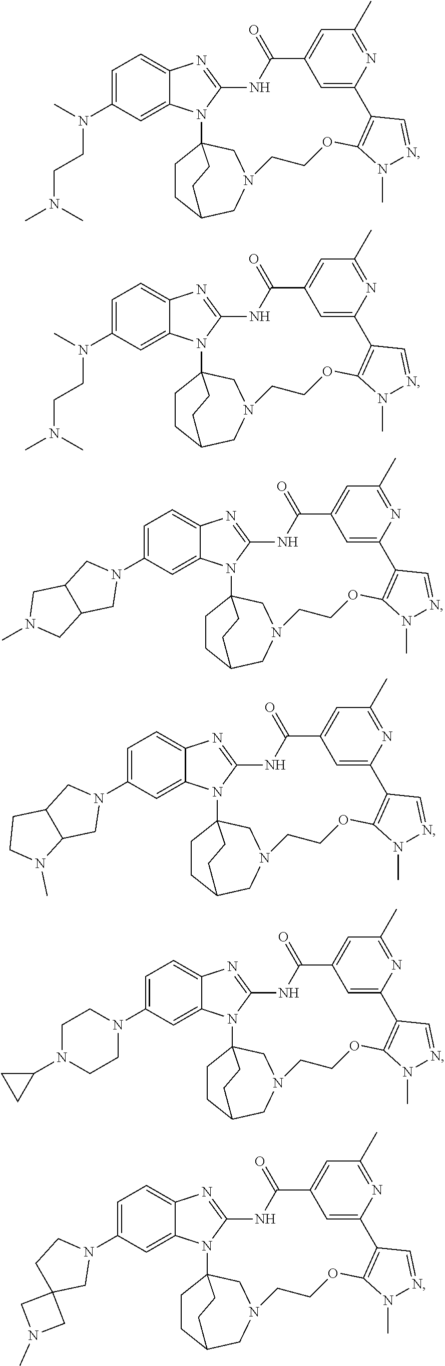

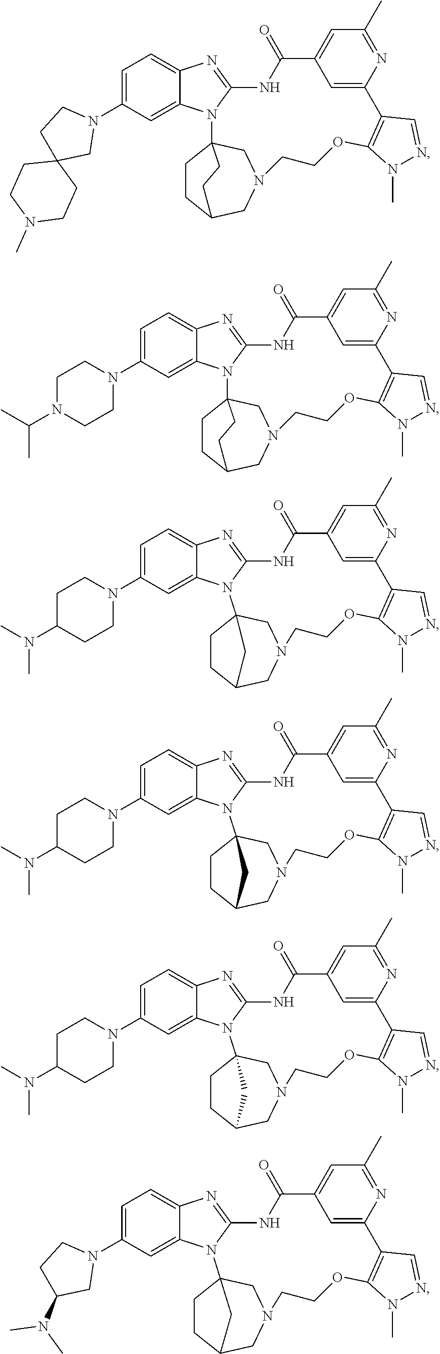

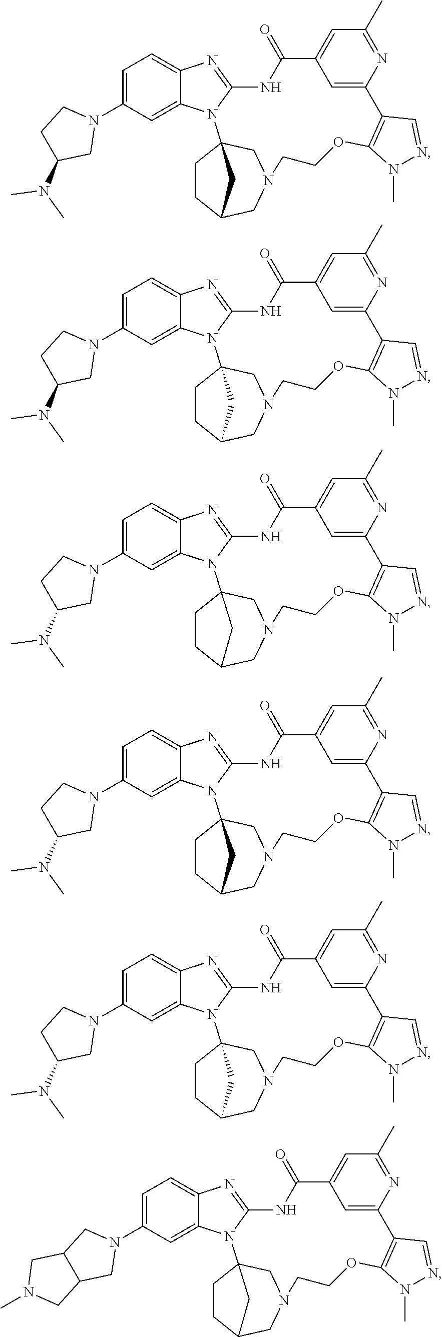

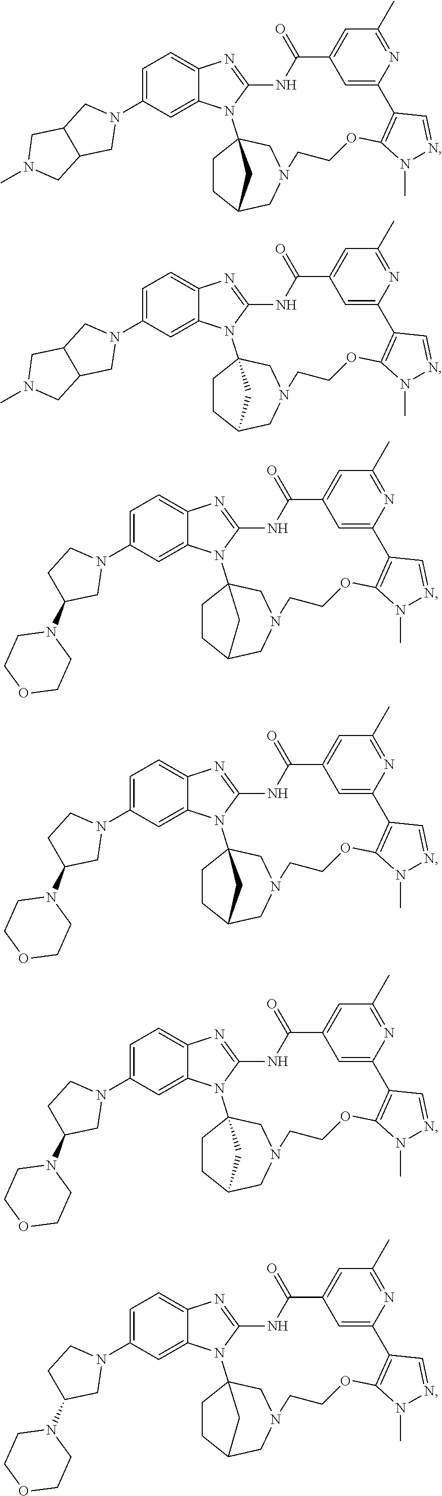

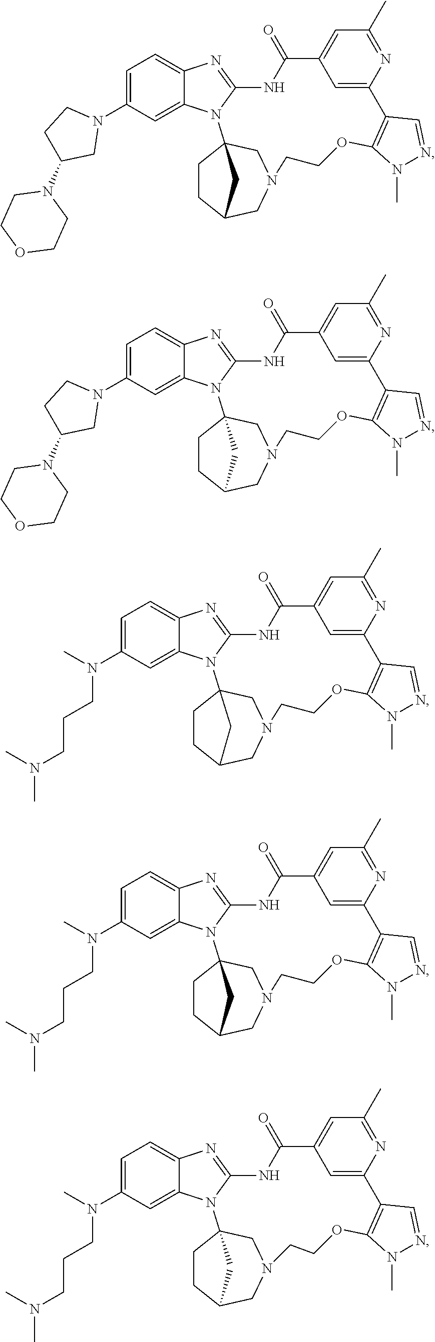 [SMILES: Cc1cc2cc(n1)-c1cnn(C)c1OCCN1C[C@@H]3CC[C@@](C3)(C1)n1c(nc3ccc(N4CC[C@@H](N5CCOCC5)C4)cc31)NC2=O ; Cc1cc2cc(n1)-c1cnn(C)c1OCCN1C[C@H]3CC[C@](C3)(C1)n1c(nc3ccc(N4CC[C@@H](N5CCOCC5)C4)cc31)NC2=O ; Cc1cc2cc(n1)-c1cnn(C)c1OCCN1CC3CCC(C3)(C1)n1c(nc3ccc(N(C)CCCN(C)C)cc31)NC2=O ; Cc1cc2cc(n1)-c1cnn(C)c1OCCN1C[C@@H]3CC[C@@](C3)(C1)n1c(nc3ccc(N(C)CCCN(C)C)cc31)NC2=O ; Cc1cc2cc(n1)-c1cnn(C)c1OCCN1C[C@H]3CC[C@](C3)(C1)n1c(nc3ccc(N(C)CCCN(C)C)cc31)NC2=O]

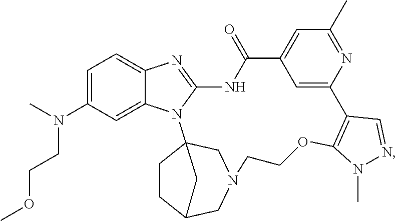 [SMILES: COCCN(C)c1ccc2nc3n(c2c1)C12CCC(CN(CCOc4c(cnn4C)-c4cc(cc(C)n4)C(=O)N3)C1)C2]

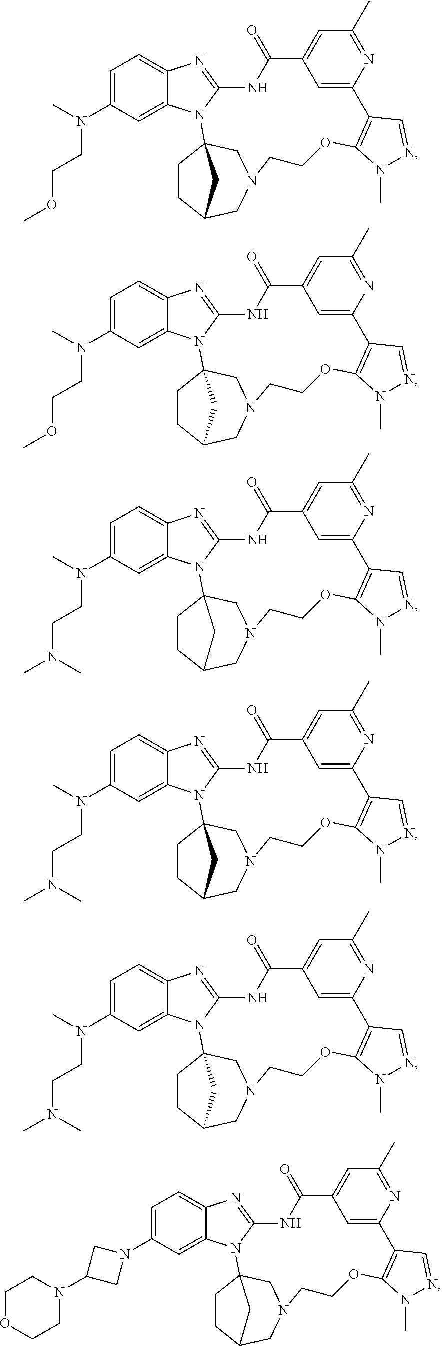

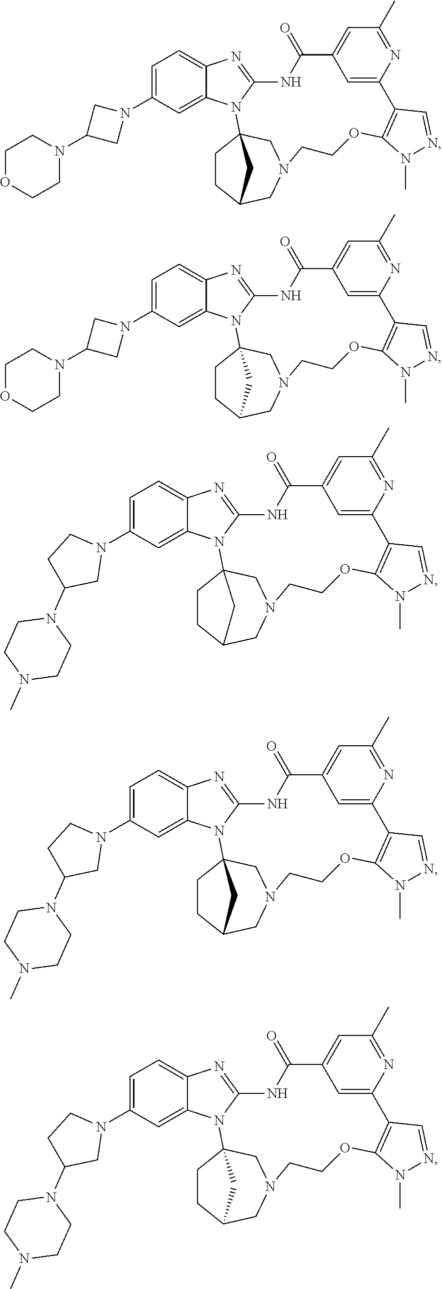

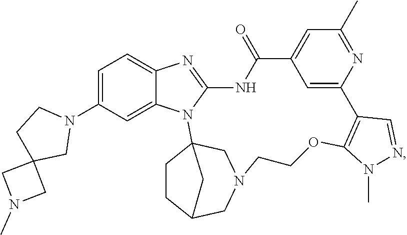 [SMILES: Cc1cc2cc(n1)-c1cnn(C)c1OCCN1CC3CCC(C3)(C1)n1c(nc3ccc(N4CCC5(CN(C)C5)C4)cc31)NC2=O]

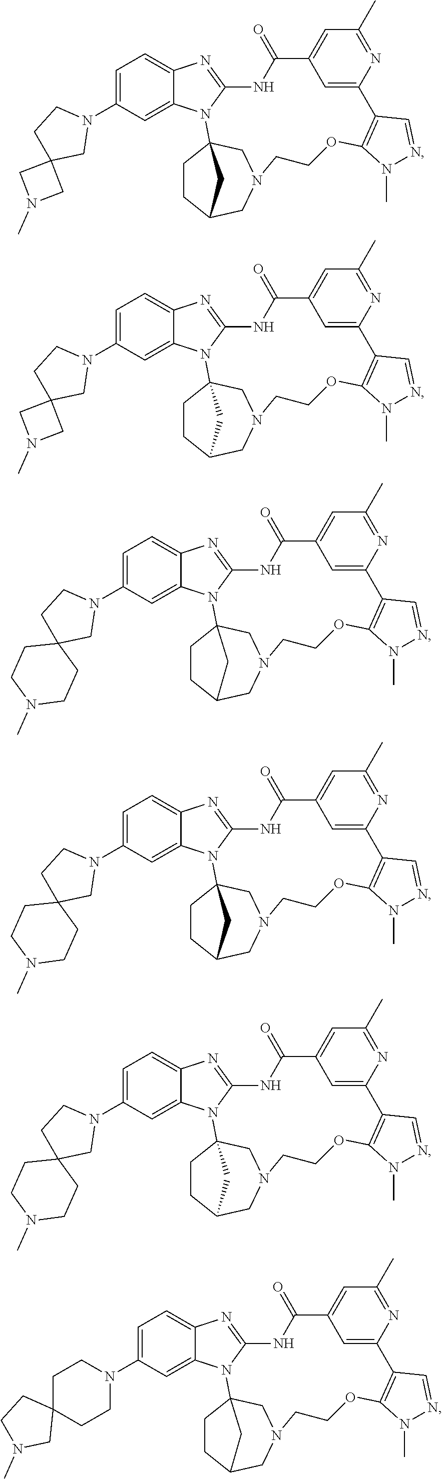

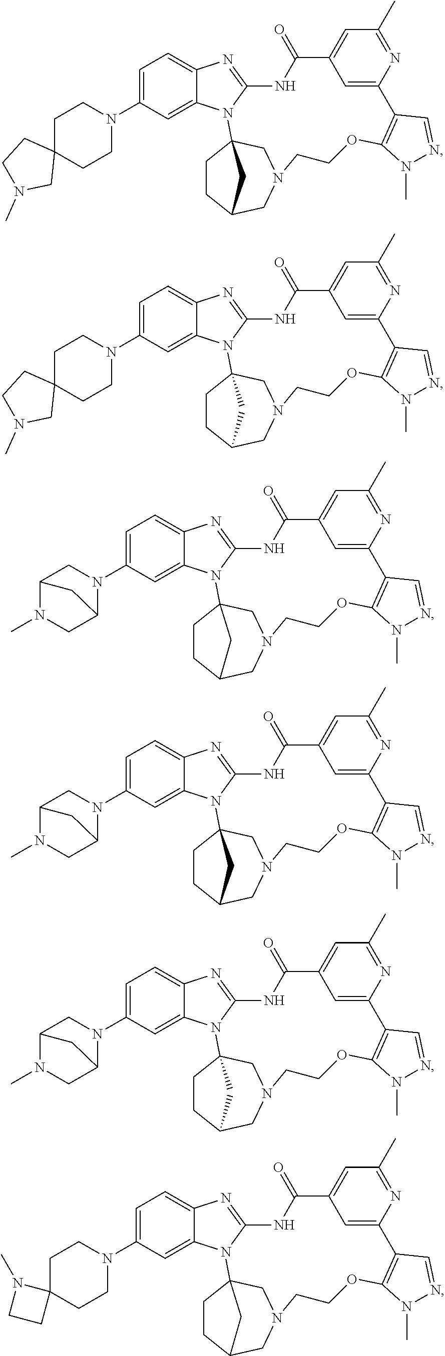

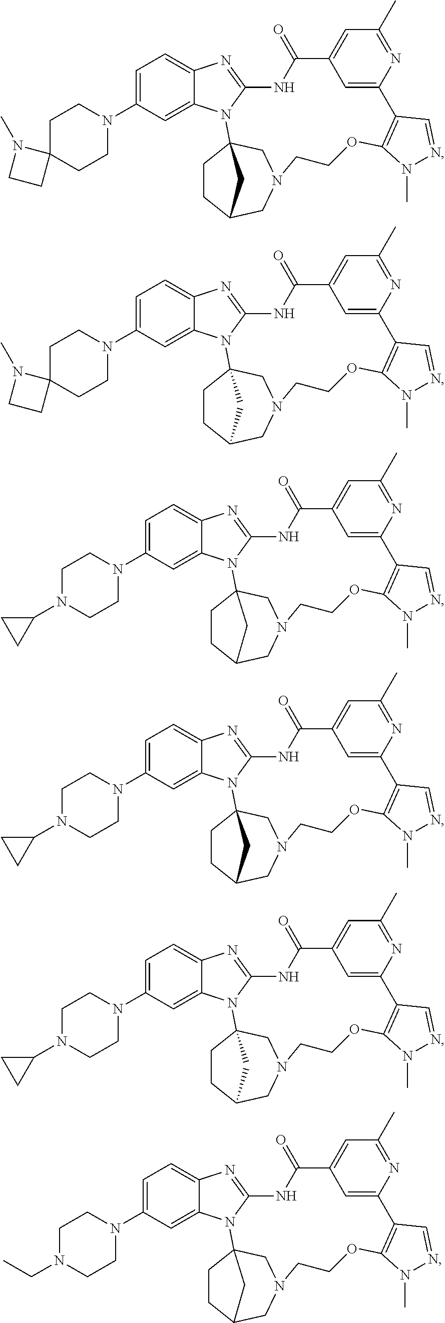

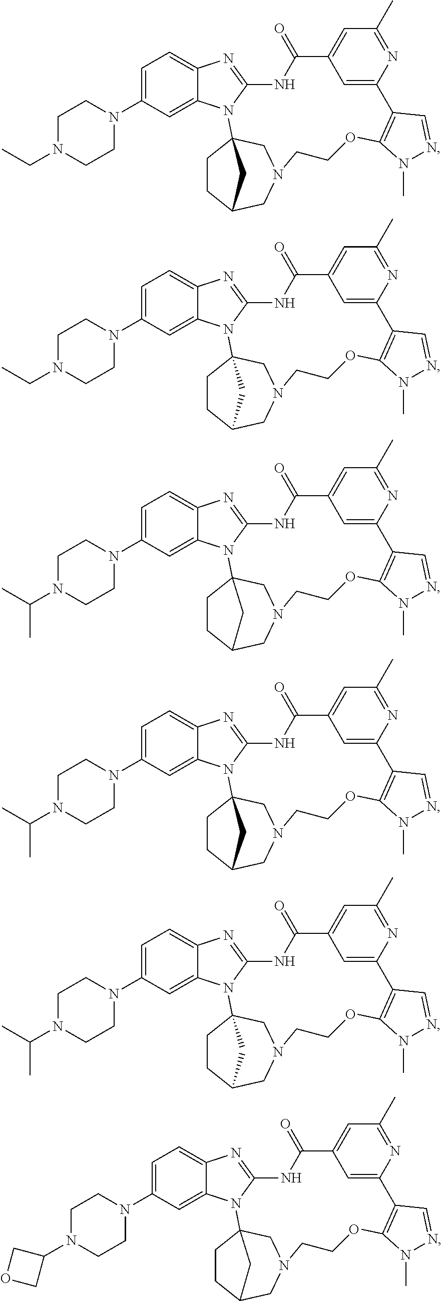

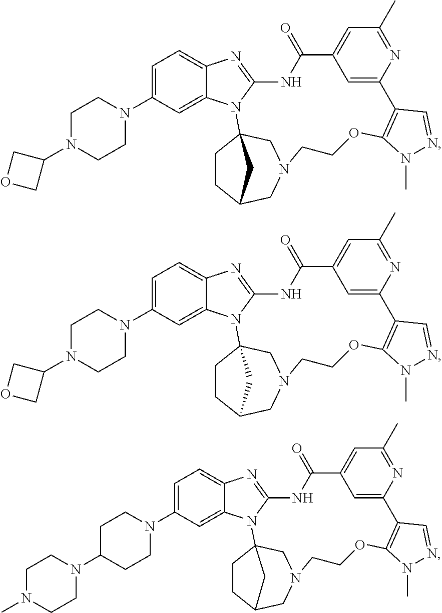

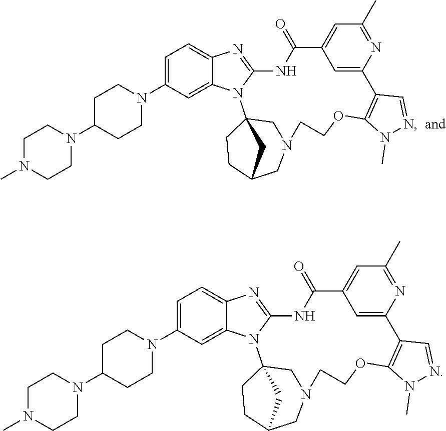

Compounds provided herein are described with reference to both generic formulae and specific compounds. In addition, the compounds of the present disclosure may exist in a number of different forms or derivatives, including but not limited to prodrugs, active metabolic derivatives (active metabolites), solvates, pharmaceutically acceptable salts or isotope derivatives, all of which are within the scope of the present disclosure.

As used herein, the term “prodrugs” refers to compounds or pharmaceutically acceptable salts thereof which, when metabolized under physiological conditions or when converted by solvolysis, yield the desired active compound. Prodrugs include, without limitation, esters, amides, carbamates, carbonates, ureides, solvates, or hydrates of the active compound. Typically, the prodrug is inactive, or less active than the active compound, but may provide one or more advantageous handling, administration, and/or metabolic properties. For example, some prodrugs are esters of the active compound; during metabolysis, the ester group is cleaved to yield the active drug. Also, some prodrugs are activated enzymatically to yield the active compound, or a compound which, upon further chemical reaction, yields the active compound. Prodrugs may proceed from prodrug form to active form in a single step or may have one or more intermediate forms which may themselves have activity or may be inactive. Preparation and use of prodrugs is discussed in T. Higuchi and V. Stella, “Pro-drugs as Novel Delivery Systems”, Vol. 14 of the A.C.S. Symposium Series, in Bioreversible Carriers in Drug Design, ed. Edward B. Roche, American Pharmaceutical Association and Pergamon Press, 1987; in Prodrugs: Challenges and Rewards, ed. V. Stella, R. Borchardt, M. Hageman, R. Oliyai, H. Maag, J. Tilley, Springer-Verlag New York, 2007, all of which are hereby incorporated by reference in their entirety.

As used herein, the term “metabolite”, e.g., active metabolite overlaps with prodrug as described above. Thus, such metabolites are pharmacologically active compounds or compounds that further metabolize to pharmacologically active compounds that are derivatives resulting from metabolic process in the body of a subject. For example, such metabolites may result from oxidation, reduction, hydrolysis, amidation, deamidation, esterification, deesterification, enzymatic cleavage, and the like, of the administered compound or salt or prodrug. Of these, active metabolites are such pharmacologically active derivative compounds. For prodrugs, the prodrug compound is generally inactive or of lower activity than the metabolic product. For active metabolites, the parent compound may be either an active compound or may be an inactive prodrug.

Prodrugs and active metabolites may be identified using routine techniques know in the art. See, e.g., Bertolini et al, 1997, J Med Chem 40:2011-2016; Shan et al., J Pharm Sci 86:756-757; Bagshawe, 1995, DrugDev Res 34:220-230; Wermuth, supra.

As used herein, the term “pharmaceutically acceptable” indicates that the substance or composition is compatible chemically and/or toxicologically, with the other ingredients comprising a formulation, and/or the subjects being treated therewith.

As used herein, the term “pharmaceutically acceptable salt”, unless otherwise indicated, includes salts that retain the biological effectiveness of the free acids and bases of the specified compound and that are not biologically or otherwise undesirable. Contemplated pharmaceutically acceptable salt forms include, but are not limited to, mono, bis, tris, tetrakis, and so on. Pharmaceutically acceptable salts are non-toxic in the amounts and concentrations at which they are administered. The preparation of such salts can facilitate the pharmacological use by altering the physical characteristics of a compound without preventing it from exerting its physiological effect. Useful alterations in physical properties include lowering the melting point to facilitate transmucosal administration and increasing the solubility to facilitate administering higher concentrations of the drug.

Pharmaceutically acceptable salts include acid addition salts such as those containing sulfate, chloride, hydrochloride, fumarate, maleate, phosphate, sulfamate, acetate, citrate, lactate, tartrate, methanesulfonate, ethanesulfonate, benzenesulfonate, p-toluenesulfonate, cyclohexylsulfamate and quinate. Pharmaceutically acceptable salts can be obtained from acids such as hydrochloric acid, maleic acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, fumaric acid, and quinic acid.

Pharmaceutically acceptable salts also include basic addition salts such as those containing benzathine, chloroprocaine, choline, diethanolamine, ethanolamine, t-butylamine, ethylenediamine, meglumine, procaine, aluminum, calcium, lithium, magnesium, potassium, sodium, ammonium, alkylamine, and zinc, when acidic functional groups, such as carboxylic acid or phenol are present. For example, see Remington's Pharmaceutical Sciences, 19^{th} ed., Mack Publishing Co., Easton, PA, Vol. 2, p. 1457, 1995; “Handbook of Pharmaceutical Salts: Properties, Selection, and Use” by Stahl and Wermuth, Wiley-VCH, Weinheim, Germany, 2002. Such salts can be prepared using the appropriate corresponding bases.

Pharmaceutically acceptable salts can be prepared by standard techniques. For example, the free-base form of a compound can be dissolved in a suitable solvent, such as an aqueous or aqueous-alcohol solution containing the appropriate acid and then isolated by evaporating the solution. Thus, if the particular compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

Similarly, if the particular compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as L-glycine, L-lysine, and L-arginine, ammonia, primary, secondary, and tertiary amines, and cyclic amines, such as hydroxyethylpyrrolidine, piperidine, morpholine or piperazine, and inorganic salts derived from sodium, calcium, potassium, magnesium, manganese, iron, copper, zinc, aluminum and lithium.

It is also to be understood that the compounds of present disclosure can exist in unsolvated forms, solvated forms (e.g., hydrated forms), and solid forms (e.g., crystal or polymorphic forms), and the present disclosure is intended to encompass all such forms.

As used herein, the term “solvate” or “solvated form” refers to solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O. Examples of solvents that form solvates include, but are not limited to, water, isopropanol, ethanol, methanol, DMSO, ethyl acetate, acetic acid, and ethanolamine.

The present disclosure is also intended to include all isotope forms of the compounds provided herein. Isotopes of an atom include atoms having the same atomic number but different mass numbers. For example, unless otherwise specified, hydrogen, carbon, nitrogen, oxygen, phosphorous, sulfur, fluorine, chlorine, bromide or iodine in the compounds of present disclosure are meant to also include their isotopes, such as but not limited to ¹H, ²H, ³H, ¹¹C, ¹²C, ¹³C, ¹⁴C, ¹⁴N, ¹⁵N, ¹⁶O, ¹⁷O, ¹⁸O, ³¹P ³²P, ³²S, ³³S, ³⁴S, ³⁶S, ¹⁷F, ¹⁸F, ¹⁹F, ³⁵Cl, ³⁷Cl, ⁷⁹Br, ⁸¹Br, ¹²⁴I, ¹²⁷I and ¹³¹I. In some embodiments, hydrogen includes protium, deuterium and tritium. In some embodiments, carbon includes ¹²C and ¹³C.

Compounds provided herein or pharmaceutically acceptable salts thereof may contain one or more asymmetric centers and thus give rise to enantiomers, diastereomers, and other stereoisomeric forms that may be defined, in terms of absolute stereochemistry, as (R)- or (S)- or, as (D)- or (L)- for amino acids, or in terms of relative configuration, as rel-(R)- or rel-(S)-. The present disclosure includes all such possible isomers, as well as their racemic and optically pure forms. Optically active (+) and (−), (R)- and (S)-, or (D)- and (L)- isomers may be prepared using chiral synthons or chiral reagents, or resolved by conventional techniques, such as, chromatography and fractional crystallization. Traditional techniques for the preparation, isolation of individual enantiomers include chiral synthesis from a suitable optically pure precursor or resolution of the racemate (or the racemate of a salt or derivative) using, for example, chiral high pressure liquid chromatography (HPLC). Wherever compounds are represented in their chiral form, it is understood that the embodiment includes, but is not limited to, the specific diastereomerically or enantiomerically enriched form. In situations that the chirality is not specified but is present, it is understood that the embodiment is intended to include either the specific diastereomerically or enantiomerically enriched form; or a racemic or scalemic mixture of such compound(s).

The term “stereoisomer” refers to a compound containing the same atoms bonded by the same bonds but having different three-dimensional structures, which are not interchangeable. The present disclosure contemplates various stereoisomers and mixtures thereof and includes “enantiomers”, which refers to two stereoisomers whose molecules are non-superimposable mirror images of one another.

The term “enantiomers” represent a pair of stereoisomers that are non-superimposable mirror images of each other. A 1:1 mixture of a pair of enantiomers is a “racemic” mixture. A mixture of enantiomers at a ratio other than 1:1 is a “scalemic” mixture.

The term “diastereoisomers” represent stereoisomers that have at least two asymmetric atoms, but which are not mirror-images of each other.

The term “tautomer” or “tautomeric form” refers to structural isomers of different energies which are interconvertible via a low energy barrier. The presence and concentrations of the isomeric forms will depend on the environment the compound is found in and may be different depending upon, for example, whether the compound is a solid or is in an organic or aqueous solution. By way of examples, proton tautomers (also known as prototropic tautomers) include interconversions via migration of a proton, such as keto-enol, amide-imidic acid, lactam-lactim, imine-enamine isomerizations and annular forms where a proton can occupy two or more positions of a heterocyclic system. Valence tautomers include interconversions by reorganization of some of the bonding electrons. Tautomers can be in equilibrium or sterically locked into one form by appropriate substitution. Compounds of the present disclosure identified by name or structure as one particular tautomeric form are intended to include other tautomeric forms unless otherwise specified.

When the compounds provided herein contain olefinic double bonds or other centers of geometric asymmetry, and unless specified otherwise, these compounds include both E and Z geometric isomers.

### Synthetic Method

The compounds provided herein can be prepared using any known organic synthesis techniques and can be synthesized according to any of numerous possible synthetic routes

Reactions for preparing compounds of the present disclosure can be carried out in suitable solvents, which can be readily selected by one skilled in the art of organic synthesis. Suitable solvents can be substantially non-reactive with starting materials (reactants), intermediates, or products at the temperatures at which the reactions are carried out, e.g. temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected by one skilled in the art.

Preparation of compounds of the present disclosure can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd Ed., Wiley & Sons, Inc., New York (1999), in P. Kocienski, Protecting Groups, Georg Thieme Verlag, 2003, and in Peter G. M. Wuts, Greene's Protective Groups in Organic Synthesis, 5^{th} Edition, Wiley, 2014, all of which are incorporated herein by reference in its entirety.

Reactions can be monitored according to any suitable method known in the art. For example, product formation can be monitored by spectroscopic means, such as nuclear magnetic resonance spectroscopy (e.g. ¹H or ¹³C), infrared spectroscopy, spectrophotometry (e.g. UV-visible), mass spectrometry, or by chromatographic methods such as high performance liquid chromatography (HPLC), liquid chromatography-mass spectroscopy (LCMS), or thin layer chromatography (TLC). Compounds can be purified by one skilled in the art by a variety of methods, including high performance liquid chromatography (HPLC) (“Preparative LC-MS Purification: Improved Compound Specific Method Optimization” Karl F. Blom, Brian Glass, Richard Sparks, Andrew P. Combs J. Combi. Chem. 2004, 6(6), 874-883, which is incorporated herein by reference in its entirety), and normal phase silica chromatography.

### Pharmaceutical Composition

In a further aspect, there is provided pharmaceutical compositions comprising one or more compounds of the present disclosure, or a pharmaceutically acceptable salt thereof. In some embodiments, the pharmaceutical compositions of the present disclosure comprise a first compound provided herein or a pharmaceutically acceptable salt thereof and one or more additional compounds of the same formula but said first compound and additional compounds are not the same molecules.

In another aspect, there is provided pharmaceutical composition comprising one or more compounds of the present disclosure, or a pharmaceutically acceptable salt thereof, and at least one pharmaceutical acceptable excipient.

In some embodiments, the pharmaceutical compositions of the present disclosure comprises a therapeutically effective amount of one or more compounds of the present disclosure or a pharmaceutically acceptable salt thereof.

In some embodiments, the pharmaceutical compositions of the present disclosure comprises a therapeutically effective amount of one or more compounds of the present disclosure or a pharmaceutically acceptable salt thereof, and at least one pharmaceutical acceptable excipient.

As used herein, the term “therapeutically effective amount” refers to an amount of a molecule, compound, or composition comprising the molecule or compound to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; the rate of administration; the therapeutic or combination of therapeutics selected for administration; and the discretion of the prescribing physician. Therapeutically effective amounts for a given situation can be determined by routine experimentation that is within the skill and judgment of the clinician.

As used herein, the term “pharmaceutical composition” refers to a formulation containing the molecules or compounds of the present disclosure in a form suitable for administration to a subject. The pharmaceutical compositions include compositions suitable adapted for oral administration, rectal administration, topical administration, parenteral (including subcutaneous, intramuscular, and intravenous) administration, sublingual administration, ocular administration, transdermal administration or nasal administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used herein includes both one and more than one such excipient. The term “pharmaceutically acceptable excipient” also encompasses “pharmaceutically acceptable carrier” and “pharmaceutically acceptable diluent”.

The particular excipient used will depend upon the means and purpose for which the compounds of the present disclosure are being applied. Solvents are generally selected based on solvents recognized by persons skilled in the art as safe to be administered to a mammal including humans. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof.

In some embodiments, suitable excipients may include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In some embodiments, suitable excipients may include one or more stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present disclosure or pharmaceutical composition thereof) or aid in the manufacturing of the pharmaceutical product (i.e., medicament). The active pharmaceutical ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980). A “liposome” is a small vesicle composed of various types of lipids, phospholipids and/or surfactant which is useful for delivery of a drug (such as the compounds disclosed herein and, optionally, a chemotherapeutic agent) to a mammal including humans. The components of the liposome are commonly arranged in a bilayer formation, similar to the lipid arrangement of biological membranes.

The pharmaceutical compositions provided herein can be in any form that allows for the composition to be administered to a subject, including, but not limited to a human, and formulated to be compatible with an intended route of administration.

A variety of routes are contemplated for the pharmaceutical compositions provided herein, and accordingly the pharmaceutical composition provided herein may be supplied in bulk or in unit dosage form depending on the intended administration route. For example, for oral, buccal, and sublingual administration, powders, suspensions, granules, tablets, pills, capsules, gelcaps, and caplets may be acceptable as solid dosage forms, and emulsions, syrups, elixirs, suspensions, and solutions may be acceptable as liquid dosage forms. For injection administration, emulsions and suspensions may be acceptable as liquid dosage forms, and a powder suitable for reconstitution with an appropriate solution as solid dosage forms. For inhalation administration, solutions, sprays, dry powders, and aerosols may be acceptable dosage form. For topical (including buccal and sublingual) or transdermal administration, powders, sprays, ointments, pastes, creams, lotions, gels, solutions, and patches may be acceptable dosage form. For vaginal administration, pessaries, tampons, creams, gels, pastes, foams and spray may be acceptable dosage form.

In some embodiments, the pharmaceutical compositions of the present disclosure may be in a form of formulation for oral administration.

In certain embodiments, the pharmaceutical compositions of the present disclosure may be in the form of tablet formulations. Suitable pharmaceutically-acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case using conventional coating agents and procedures well known in the art.

In certain embodiments, the pharmaceutical compositions of the present disclosure may be in a form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

In certain embodiments, the pharmaceutical compositions of the present disclosure may be in the form of aqueous suspensions, which generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), coloring agents, flavoring agents, and/or sweetening agents (such as sucrose, saccharine or aspartame).

In certain embodiments, the pharmaceutical compositions of the present disclosure may be in the form of oily suspensions, which generally contain suspended active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

In certain embodiments, the pharmaceutical compositions of the present disclosure may be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

In certain embodiments, the pharmaceutical compositions provided herein may be in the form of syrups and elixirs, which may contain sweetening agents such as glycerol, propylene glycol, sorbitol, aspartame or sucrose, a demulcent, a preservative, a flavoring and/or coloring agent.

In some embodiments, the pharmaceutical compositions of the present disclosure may be in a form of formulation for injection administration.

In certain embodiments, the pharmaceutical compositions of the present disclosure may be in the form of a sterile injectable preparation, such as a sterile injectable aqueous or oleaginous suspension. This suspension may be formulated according to the known art using those suitable dispersing or wetting agents and suspending agents, which have been mentioned above. The sterile injectable preparation may also be a sterile injectable solution or suspension in a non-toxic parenterally acceptable diluent or solvent, such as a solution in 1,3-butanediol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose, any bland fixed oil may be employed including synthetic mono- or diglycerides. In addition, fatty acids such as oleic acid may likewise be used in the preparation of injectables.

In some embodiments, the pharmaceutical compositions of the present disclosure may be in a form of formulation for inhalation administration.

In certain embodiments, the pharmaceutical compositions of the present disclosure may be in the form of aqueous and nonaqueous (e.g., in a fluorocarbon propellant) aerosols containing any appropriate solvents and optionally other compounds such as, but not limited to, stabilizers, antimicrobial agents, antioxidants, pH modifiers, surfactants, bioavailability modifiers and combinations of these. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols.

In some embodiments, the pharmaceutical compositions of the present disclosure may be in a form of formulation for topical or transdermal administration. In certain embodiments, the pharmaceutical compositions provided herein may be in the form of creams, ointments, gels and aqueous or oily solutions or suspensions, which may generally be obtained by formulating an active ingredient with a conventional, topically acceptable excipients such as animal and vegetable fats, oils, waxes, paraffins, starch, tragacanth, cellulose derivatives, polyethylene glycols, silicones, bentonites, silicic acid, talc and zinc oxide, or mixtures thereof.

In certain embodiments, the pharmaceutical compositions provided herein may be formulated for administration ocularly. In certain embodiments, the pharmaceutical compostions provided herein may be in the form of ophthalmic formulation, such as eye ointments, powders, solutions and the like. In certain embodiments, ophthalmic formulations are prepared at a comfortable pH with an appropriate buffer system.

Besides those representative dosage forms described above, pharmaceutically acceptable excipients and carriers are generally known to those skilled in the art and are thus included in the present disclosure. Such excipients and carriers are described, for example, in “Remingtons Pharmaceutical Sciences” Mack Pub. Co., New Jersey (1991), in “Remington: The Science and Practice of Pharmacy”, Ed. University of the Sciences in Philadelphia, 21^{st} Edition, LWW (2005), which are incorporated herein by reference.

The dosage regimen for the compounds provided herein will vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. A physician or veterinarian can determine and prescribe the effective amount of the drug required to prevent, counter, or arrest the progress of the disorder.

In some embodiments, the pharmaceutical compositions of the present disclosure can be formulated so that a dosage of between 0.001-1000 mg/kg body weight/day, for example, 0.01-800 mg/kg body weight/day, 0.01-700 mg/kg body weight/day, 0.01-600 mg/kg body weight/day, 0.01-500 mg/kg body weight/day, 0.01-400 mg/kg body weight/day, 0.01-300 mg/kg body weight/day, 0.1-200 mg/kg body weight/day, 0.1-150 mg/kg body weight/day, 0.1-100 mg/kg body weight/day, 0.5-100 mg/kg body weight/day, 0.5-80 mg/kg body weight/day, 0.5-60 mg/kg body weight/day, 0.5-50 mg/kg body weight/day, 1-50 mg/kg body weight/day, 1-45 mg/kg body weight/day, 1-40 mg/kg body weight/day, 1-35 mg/kg body weight/day, 1-30 mg/kg body weight/day, 1-25 mg/kg body weight/day of the compounds provided herein, or a pharmaceutically acceptable salt thereof, can be administered. In some instances, dosage levels below the lower limit of the aforesaid range may be more than adequate, while in other cases still larger doses may be employed without causing any harmful side effect, provided that such larger doses are first divided into several small doses for administration throughout the day. For further information on routes of administration and dosage regimes, see Chapter 25.3 in Volume 5 of Comprehensive Medicinal Chemistry (Corwin Hansch; Chairman of Editorial Board), Pergamon Press 1990, which is specifically incorporated herein by reference.

In some embodiments, the pharmaceutical compositions of the present disclosure can be formulated as a single dosage form. The amount of the compounds provided herein in the single dosage form will vary depending on the subject treated and particular mode of administration.

In some embodiments, dosage forms suitable for administration may contain from about 1 mg to about 1000 mg of active ingredient per dosage unit. In these pharmaceutical compositions the active ingredient will ordinarily be present in an amount of about 0.1-95% by weight based on the total weight of the composition.

In some embodiments, the pharmaceutical compositions of the present disclosure can be formulated as short-acting, fast-releasing, long-acting, and sustained-releasing. Accordingly, the pharmaceutical formulations of the present disclosure may also be formulated for controlled release or for slow release.

In some embodiments, a dose of the compounds provided herein or the pharmaceutical compositions provided herein is administered to a subject every day, every other day, every couple of days, every third day, once a week, twice a week, three times a week, or once every two weeks. If desired, the effective daily dose of the active compound may be administered as two, three, four, five, six or more sub-doses administered separately at appropriate intervals throughout the day, optionally, in unit dosage forms. In some embodiments, a dose of the compounds provided herein or the pharmaceutical compositions provided herein is administered for 2 days, 3 days, 5 days, 7 days, 14 days, 21 days, 1 month, 2 months, 2.5 months, 3 months, 4 months, 5 months, 6 months or more.

In a further aspect, there is also provided veterinary compositions comprising one or more molecules or compounds of the present disclosure or pharmaceutically acceptable salts thereof and a veterinary carrier. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

The pharmaceutical compositions or veterinary compositions may be packaged in a variety of ways depending upon the method used for administering the drug. For example, an article for distribution can include a container having deposited therein the compositions in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings. The compositions may also be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described.

In some embodiments, the pharmaceutical composition of the present disclosure comprising one or more compounds provided herein or pharmaceutically acceptable salts thereof further comprises one or more additional therapeutically active agents.

The additional therapeutically active agents have complementary activities to the compound provided herein such that they do not adversely affect each other. Such agents are suitably present in combination in amounts that are effective for the purpose intended.

In certain embodiments, the additional therapeutic agent is selected from EGFR TKIs, EGFR antibodies, MEK inhibitors, c-MET inhibitors, mitotic kinase inhibitors, immunotherapeutic agents, anti-angiogenic agents, apoptosis inducers, mTOR inhibitors, histone deacetylase inhibitors, IL6 inhibitors, JAK inhibitors.

Examples of EGFR TKIs include, for example, afatinib, erlotinib, gefitinib, lapatinib, dacomitinib, osimertinib, olmutinib, nazartinib, and AC0010.

Examples of EGFR antibodies include, for example, cetuximab, panitumumab, and necitumumab.

Examples of MEK inhibitors include trametinib, cobimetinib, binimetinib, selumetinib, and refametinib.

Examples of c-MET inhibitors include, for example, savolitinib, cabozantinib, foretinib and MET antibodies (such as emibetuzumab).

Examples of mitotic kinase inhibitors include CDK4/6 inhibitors such as palbociclib, ribociclib, abemaciclib.

Examples of immunotherapeutic agents include immune checkpoint inhibitors such as anti-CTLA4 mAb, anti-PD1 mAb, anti-PD-L1 mAb, anti-PD-L2 mAb, anti-LAG3 mAb, anti-TM3 mAb, preferred anti-PD1 mAb, ipilimumab, nivolumab, pembrolizumab, atezolizumab, avelumab, durvalumab, pidilizumab, PDR-001 and immune modulator such as CD73 inhibitors or CD73 inhibitory antibodies.

Examples of anti-angiogenic agents include, for example, bevacizumab, nintedanib.

Examples of apoptosis inducers include Bcl-2 inhibitors (such as venetoclax, obatoclax, navitoclax), Mcl-1 inhibitors (such as AZD-5991, AMG-176, S-64315).

Examples of mTOR inhibitors include, for example, rapamycin, temsirolimus, everolimus, ridaforolimus.

Examples of histone deacetylase inhibitors include, for example, panobinostat, entinostat, romidepsin, and vorinostat.

Examples of IL6 inhibitors include, for example, tocilizumab, siltuximab, olokizumab, elsilimomab, clazakizumab, sirukumab, levilimab, ARGX-109, FE301, FM101.

Examples of JAK inhibitors include, for example, baricitinib, ruxolitinib, tofacitinib, oclacitinib, baricitinib, peficitinib, fedratinib, upadacitinib, filgotinib, delgocitinib, abrocitinib.

The additional therapeutic agent or agents may be administered simultaneously or sequentially with the compounds provided herein. Sequential administration includes administration before or after the compounds provided herein. In some embodiments, the additional therapeutic agent or agents may be administered in the same composition as the compounds provided herein. In other embodiments, there may be an interval of time between administration of the additional therapeutic agent and the compounds provided herein.

In some embodiments, the administration of an additional therapeutic agent with a compound provided herein may enable lower doses of the other therapeutic agents and/or administration at less frequent intervals.

### Method for Treatment

Compounds of the present disclosure and pharmaceutical composition comprising the same are capable of inhibiting EGFR, and thus can be useful for inhibiting EGFR activity in a subject in need thereof, and for preventing or treating EGFR-related disorders.

In a further aspect, the present disclosure provides a method of treating EGFR-related disorders, comprising administering an effective amount of the compound or a pharmaceutically acceptable salt thereof or the pharmaceutical composition provided herein to a subject in need thereof.

As used herein, the term “treating”, “treatment” or “therapy” is intended to have its normal meaning of dealing with a disease in order to entirely or partially relieve one, some or all of its symptoms, or to correct or compensate for the underlying pathology, thereby achieving beneficial or desired clinical results. For purposes of this disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treating” can also mean prolonging survival as compared to expected survival if not receiving it. Those in need of therapy include those already with the condition or disorder as well as those prone to have the condition or disorder or those in which the condition or disorder is to be prevented.

As used herein, the term “preventing”, “prevention” or “prophylaxis” is intended to have its normal meaning and includes primary prophylaxis to prevent the development of the disease and secondary prophylaxis whereby the disease has already developed and the patient is temporarily or permanently protected against exacerbation or worsening of the disease or the development of new symptoms associated with the disease.

In some embodiments, the compounds or pharmaceutically acceptable salts thereof and the compositions provided herein may be used for the treatment of a wide variety of EGFR-related disorders including cancer, autoimmune diseases, etc.

In certain embodiments, the compounds or pharmaceutically acceptable salts thereof and the compositions provided herein may be used for treating cancers, including lung cancer (e.g., non-small cell lung cancer), brain cancers, colorectal cancer, bladder cancer, urothelial cancer, breast cancer, prostate cancer, ovarian cancer, head and neck cancer, pancreatic cancer, gastric cancer and mesothelioma, including metastasis (in particular brain metastasis), etc.

In certain embodiments, the compounds or pharmaceutically acceptable salts thereof and the compositions provided herein may be used for treating autoimmune diseases including rheumatoid arthritis, graft versus host disease, systemic lupus erythromatosis (SLE), scleroderma, multiple sclerosis, diabetes, organ rejection, inflammatory bowel disease, psoriasis, and other afflictions.

The concentration and route of administration to the subject will vary depending on the EGFR-related disorders to be treated. In certain embodiments, the administering is conducted via a route selected from the group consisting of parenteral, intraperitoneal, intradermal, intracardiac, intraventricular, intracranial, intracerebrospinal, intrasynovial, intrathecal administration, intramuscular injection, intravitreous injection, intravenous injection, intra-arterial injection, oral, buccal, sublingual, transdermal, topical, intratracheal, intrarectal, subcutaneous, and ocular administration.

Embodiments of the various aspects provided by the present disclosure are also described in any of the following paragraphs.

Embodiment 1: A compound of Formula (I):

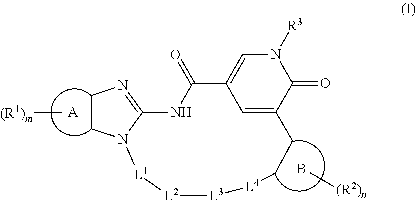

or a pharmaceutically acceptable salt thereof, wherein:
Ring A is selected from the group consisting of cycloalkyl, heterocyclyl, aryl and heteroaryl;
Ring B is selected from the group consisting of cycloalkyl, heterocyclyl, aryl, and heteroaryl;
L¹ is selected from the group consisting of a bond, alkyl, alkenyl, alkynyl, heteroalkyl, heteroalkenyl and heteroalkynyl, wherein the alkyl, alkenyl, alkynyl, heteroalkyl, heteroalkenyl and heteroalkynyl are optionally substituted with one or more groups independently selected from hydroxyl, halogen, cyano and amino;
L² is selected from the group consisting of a bond, N(R^{A}), alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more R^{B};
wherein R^{A} is selected from a group consisting of hydrogen, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, amino, alkyl and haloalkyl;
wherein each R^{B} is independently selected from a group consisting of hydroxyl, halogen, cyano, amino, alkyl, alkenyl, alkynyl, heteroalkyl, heteroalkenyl, cycloalkyl, heterocyclyl, aryl and heteroaryl;
L³ is selected from the group consisting of a bond, alkyl, alkenyl, alkynyl, heteroalkyl, heteroalkenyl and heteroalkynyl, wherein the alkyl, alkenyl, alkynyl, heteroalkyl, heteroalkenyl and heteroalkynyl are optionally substituted with one or more groups independently selected from hydroxyl, halogen, cyano and amino;
L⁴ is selected from O, S, or N(R^{C});
wherein R^{C} is selected from a group consisting of hydrogen, alkyl, alkenyl, alkynyl, haloalkyl, heteroalkyl, heteroalkenyl, heteroalkynyl, cycloalkyl, heterocyclyl, aryl, and heteroaryl;
each R¹ is independently selected from the group consisting of hydroxy, halogen, cyano, amino, —N(R^{D})₂, alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more R^{E};
wherein each R^{D} is independently selected from a group consisting of hydrogen, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, —N(R^{F})₂ or —OR^{G};
wherein each R^{E} is independently selected from a group consisting of hydrogen, hydroxyl, halogen, cyano, amino, —N(R^{F})₂, -alkyl-N(R^{F})₂, —C(O)OR^{G}, alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, amino and alkyl;
wherein each of R^{F} and R^{G} is independently selected from a group consisting of alkyl, alkenyl, alkynyl, heteroalkyl, heteroalkenyl, heteroalkynyl, cycloalkyl, heterocyclyl, aryl and heteroaryl;
each R² is independently selected from the group consisting of hydrogen, hydroxy, halogen, cyano, amino, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, amino, alkyl and haloalkyl;
R³ is selected from the group consisting of hydrogen, alkyl, alkenyl, alkynyl, heteroalkyl, heteroalkenyl, heteroalkynyl, cycloalkyl and heterocyclyl, wherein the alkyl, alkenyl, alkynyl, heteroalkyl, heteroalkenyl, heteroalkynyl, cycloalkyl and heterocyclyl are optionally substituted with one or more groups independently selected from hydroxyl, halogen, cyano, amino, alkyl and haloalkyl;
m is an integer from 0 to 5; and
n is an integer from 0 to 4.

Embodiment 2. The compound of embodiment 1, or a pharmaceutically acceptable salt thereof, wherein Ring A is aryl or heteroaryl.

Embodiment 3. The compound of embodiment 2, or a pharmaceutically acceptable salt thereof, wherein Ring A is selected from a group consisting of furanyl, thiophenyl, pyrrolyl, phenyl, pyridinyl, pyranyl, pyrimidinyl, pyridazinyl, pyrazinyl and tetrahydroisoquinoline.

Embodiment 4. The compound of embodiment 3, or a pharmaceutically acceptable salt thereof, wherein Ring A is selected from a group consisting of

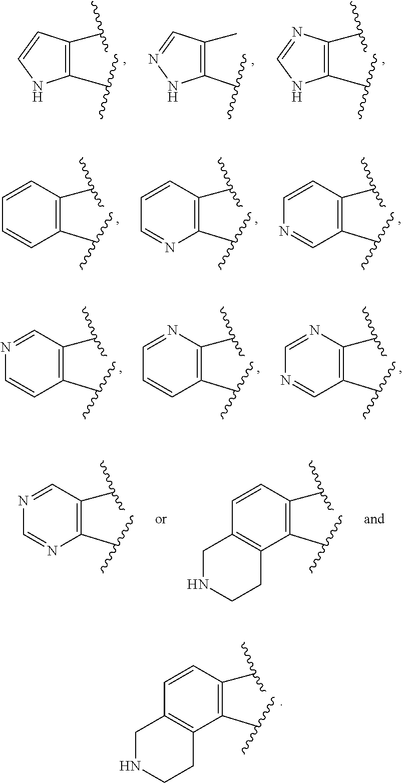

Embodiment 5. The compound of embodiment 1, or a pharmaceutically acceptable salt thereof, wherein Ring B is aryl or heteroaryl.

Embodiment 6. The compound of embodiment 5, or a pharmaceutically acceptable salt thereof, wherein Ring B is phenyl or pyrazolyl.

Embodiment 7. The compound of embodiment 6, or a pharmaceutically acceptable salt thereof, wherein Ring B is

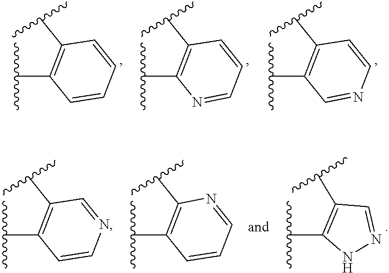

Embodiment 8. The compound of embodiment 1, or a pharmaceutically acceptable salt thereof, wherein L¹ is a bond.

Embodiment 9. The compound of embodiment 8, or a pharmaceutically acceptable salt thereof, wherein L¹ is alkyl.

Embodiment 10. The compound of embodiment 9, or a pharmaceutically acceptable salt thereof, wherein L¹ is selected from a group consisting of

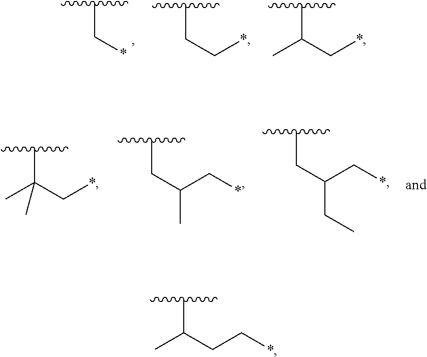

and *, wherein * end of L¹ is connected to L².

Embodiment 11. The compound of embodiment 1, or a pharmaceutically acceptable salt thereof, wherein L² is a bond.

Embodiment 12. The compound of embodiment 1, or a pharmaceutically acceptable salt thereof, wherein L² is N(R^{A}), and R^{A} is selected from alkyl or heterocyclyl, wherein the alkyl or heterocyclyl are are optionally substituted with one or more halogen or alkyl.

Embodiment 13. The compound of embodiment 12, or a pharmaceutically acceptable salt thereof, wherein R^{A} is ethyl, difluoroethyl, trifluoroethyl or oxetanyl.

Embodiment 14. The compound of embodiment 1, or a pharmaceutically acceptable salt thereof, wherein L² is cycloalkyl optionally substituted with one or more R^{B}.

Embodiment 15. The compound of embodiment 14, or a pharmaceutically acceptable salt thereof, wherein L² is

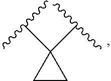 [SMILES: *C1(*)CC1]

which is optionally substituted with one or more R^{B}.

Embodiment 16. The compound of embodiment 1, or a pharmaceutically acceptable salt thereof, wherein L² is heterocyclyl optionally substituted with one or more R^{B}.

Embodiment 17. The compound of embodiment 16, or a pharmaceutically acceptable salt thereof, wherein the heterocyclyl is selected from a group consisting of

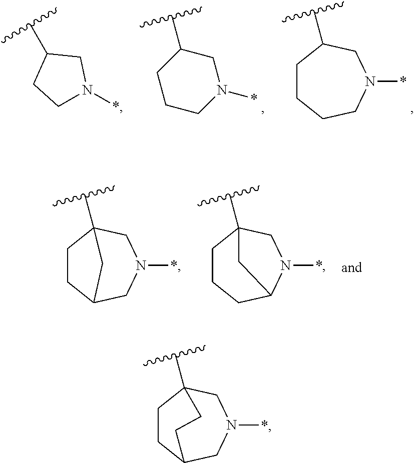

each of which is optionally substituted with one or more R^{B}, wherein * end of L² is connected to L³.

Embodiment 18. The compound of embodiment 16 or 17, or a pharmaceutically acceptable salt thereof, wherein R^{B} is alkyl.

Embodiment 19. The compound of embodiment 19, or a pharmaceutically acceptable salt thereof, wherein R^{B} is methyl.

Embodiment 20. The compound of embodiment 1, or a pharmaceutically acceptable salt thereof, wherein L² is selected from a group consisting of:

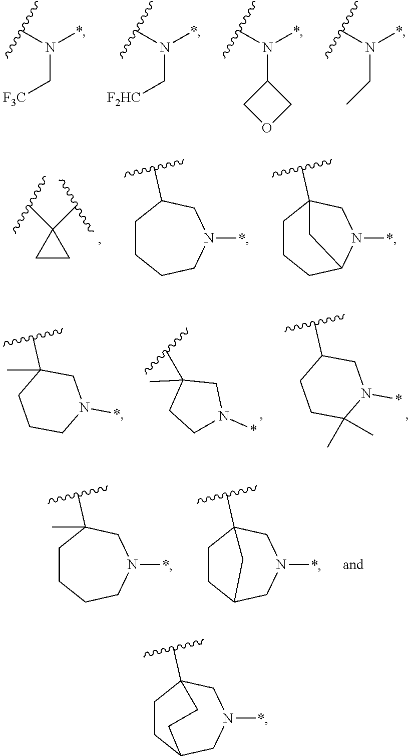

wherein * end of L² is connected to L³.

Embodiment 21. The compound of embodiment 1, or a pharmaceutically acceptable salt thereof, wherein L³ is alkyl.

Embodiment 22. The compound of embodiment 21, or a pharmaceutically acceptable salt thereof, wherein L³ is ethyl.

Embodiment 23. The compound of embodiment 1, or a pharmaceutically acceptable salt thereof, wherein L⁴ is O or NH.

Embodiment 24. The compound of embodiment 1, or a pharmaceutically acceptable salt thereof, wherein L¹ is a bond and L² is a bond or heterocyclyl optionally substituted with one or more R^{B}.

Embodiment 25. The compound of embodiment 1, or a pharmaceutically acceptable salt thereof, wherein L¹ is alkyl and L² is a bond, N(R^{A}), or cycloalkyl optionally substituted with one or more R^{B}.

Embodiment 26. The compound of embodiment 1, or a pharmaceutically acceptable salt thereof, wherein R¹ is halogen and m is 1.

Embodiment 27. The compound of embodiment 26, or a pharmaceutically acceptable salt thereof, wherein R¹ is bromo.

Embodiment 28. The compound of embodiment 1, or a pharmaceutically acceptable salt thereof, wherein R¹ is —N(R^{D})₂ and m is 1.

Embodiment 29. The compound of embodiment 28, or a pharmaceutically acceptable salt thereof, wherein each R^{D} is independently alkyl optionally substituted with one or more groups independently selected from —N(R^{F})₂ or —OR^{G}.

Embodiment 30. The compound of embodiment 29, or a pharmaceutically acceptable salt thereof, wherein each R^{D} is independently methyl, methoxyethyl, or N,N-dimethylaminopropyl.

Embodiment 31. The compound of embodiment 1, or a pharmaceutically acceptable salt thereof, wherein R¹ is heterocyclyl optionally substituted with one or more R^{E} and m is 1.

Embodiment 32. The compound of embodiment 31, or a pharmaceutically acceptable salt thereof, wherein the heterocyclyl is selected from a group consisting of

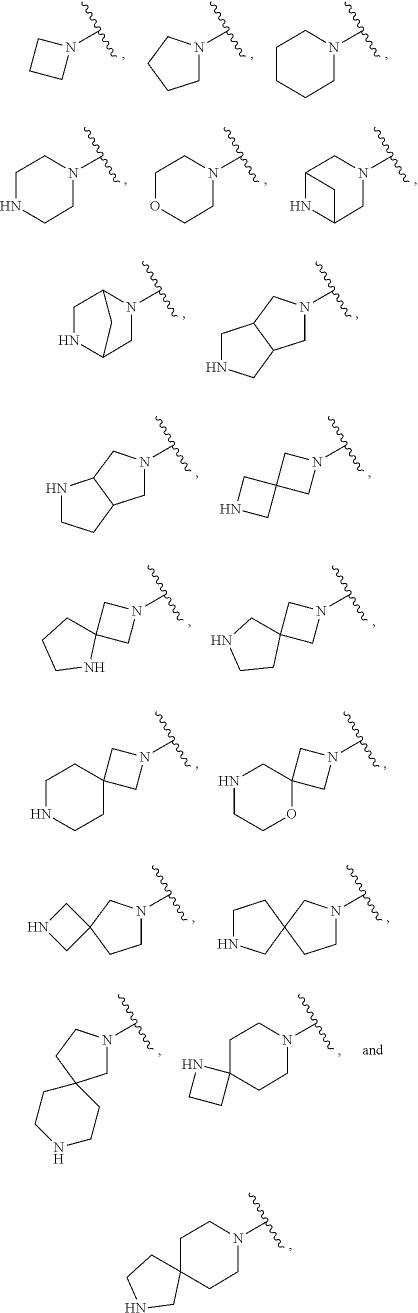

each of which is optionally substituted with one or more R^{E}.

Embodiment 33. The compound of embodiment 31, or a pharmaceutically acceptable salt thereof, wherein R^{E} is halogen.

Embodiment 34. The compound of embodiment 32, or a pharmaceutically acceptable salt thereof, wherein R^{E} is F.

Embodiment 35. The compound of embodiment 31, or a pharmaceutically acceptable salt thereof, wherein R^{E} is —N(R^{F})₂, -alkyl-N(R^{F})₂, —C(O)OR^{G}, or alkyl optionally substituted with one or more halogen.

Embodiment 36. The compound of embodiment 35, or a pharmaceutically acceptable salt thereof, wherein each of R^{F} and R^{G} is alkyl.

Embodiment 37. The compound of embodiment 35, or a pharmaceutically acceptable salt thereof, wherein each of R^{F} and R^{G} is C₁₋₃ alkyl.

Embodiment 38. The compound of embodiment 35, or a pharmaceutically acceptable salt thereof, wherein R^{E} is selected from a group consisting of —N(CH₃)₂, —C₁₋₃ alkyl-N(CH₃)₂, —C(O)O(tert-butyl), methyl, ethyl, or trifluoroethyl.

Embodiment 39. The compound of embodiment 31, or a pharmaceutically acceptable salt thereof, wherein R^{E} is cycloalkyl or heterocyclyl, wherein the cycloalkyl and heterocyclyl are optionally substituted with one or more alkyl or halogen.

Embodiment 40. The compound of embodiment 39, or a pharmaceutically acceptable salt thereof, wherein R^{E} is selected from cyclopropyl, morpholinyl, piperazinyl, oxetyl or azetidinyl, each of which is optionally substituted with one or more alkyl or halogen.

Embodiment 41. The compound of embodiment 1, or a pharmaceutically acceptable salt thereof, wherein R¹ is selected from the group consisting of

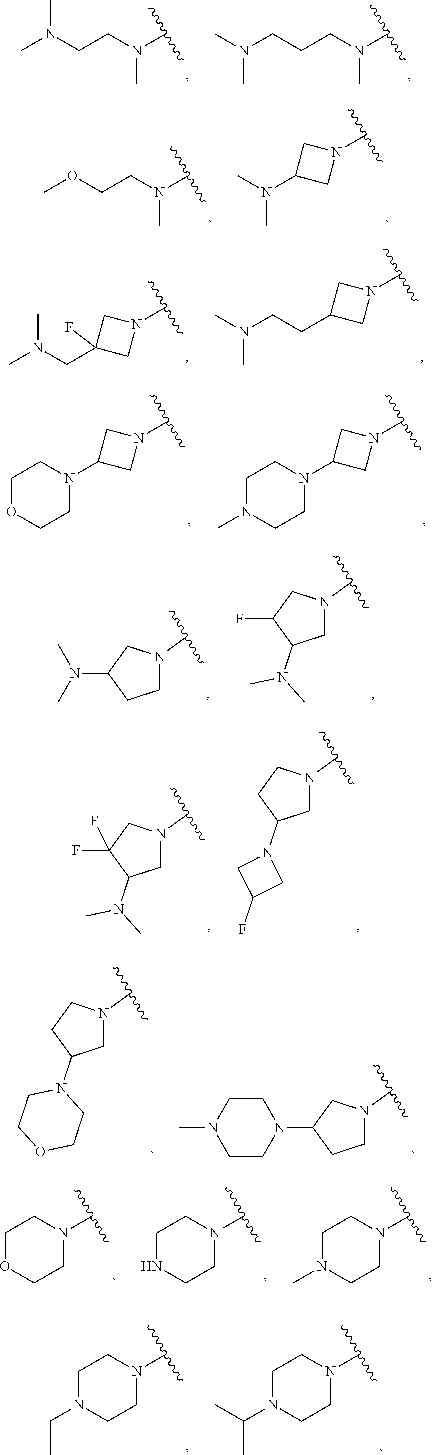

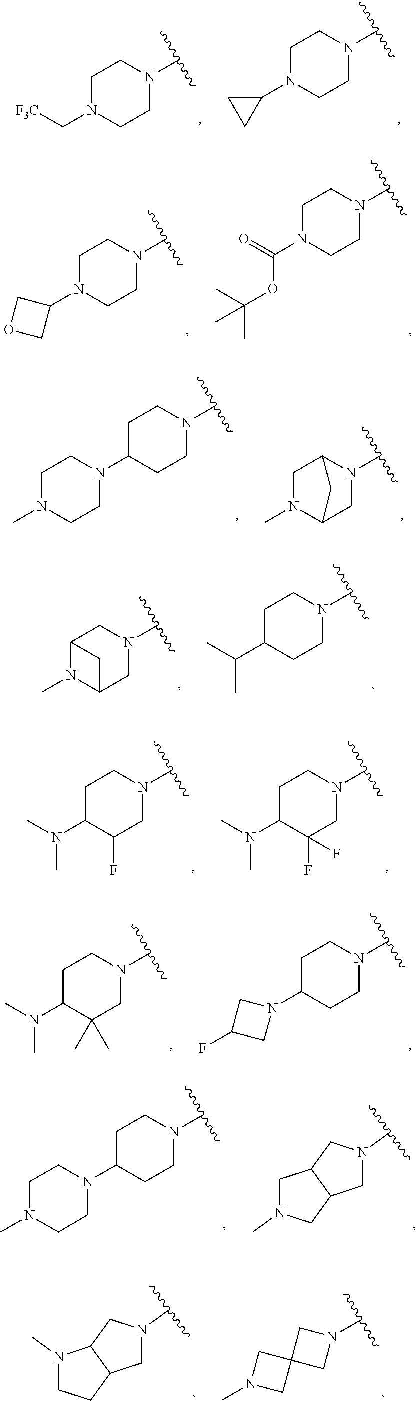

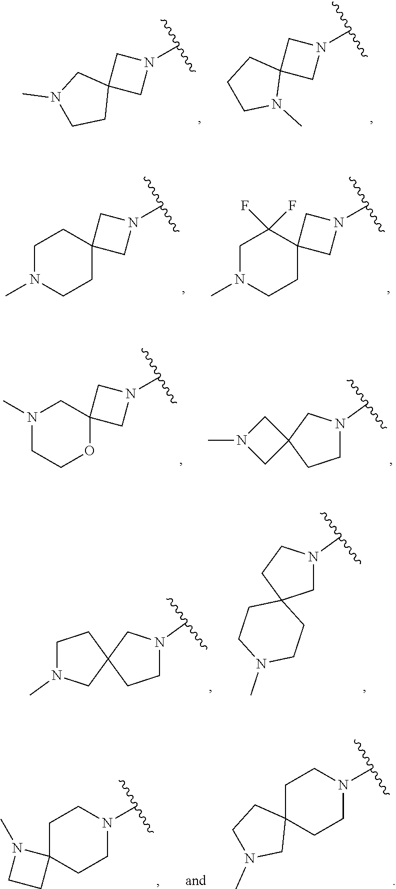

Embodiment 42. The compound of embodiment 1, or a pharmaceutically acceptable salt thereof, wherein R² is halogen and n is 1 or 2.

Embodiment 43. The compound of embodiment 42, or a pharmaceutically acceptable salt thereof, wherein R² is F.

Embodiment 44. The compound of embodiment 1, or a pharmaceutically acceptable salt thereof, wherein R² is alkyl or cycloalkyl, wherein the alkyl and cycloalkyl are optionally substituted with one or more halogen.

Embodiment 45. The compound of embodiment 44, or a pharmaceutically acceptable salt thereof, wherein R² is methyl, ethyl, cyclopropyl or trifluoroethyl.

Embodiment 46. The compound of embodiment 1, or a pharmaceutically acceptable salt thereof, wherein R³ is alkyl or cycloalkyl.

Embodiment 47. The compound of embodiment 46, or a pharmaceutically acceptable salt thereof, wherein R³ is methyl, ethyl or cyclopropyl.

Embodiment 48. The compound of embodiment 1, or a pharmaceutically acceptable salt thereof, wherein m is 0, 1 or 2.

Embodiment 49. The compound of embodiment 1, or a pharmaceutically acceptable salt thereof, wherein n is 0 or 1.

Embodiment 50. The compound of embodiment 1, or a pharmaceutically acceptable salt thereof, wherein the compound is selected from the group consisting of:

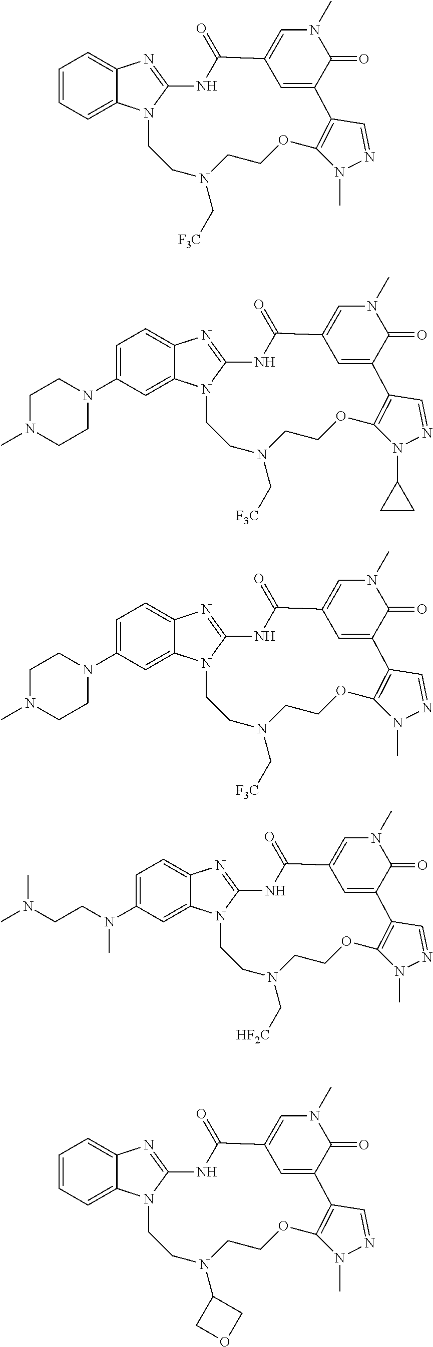

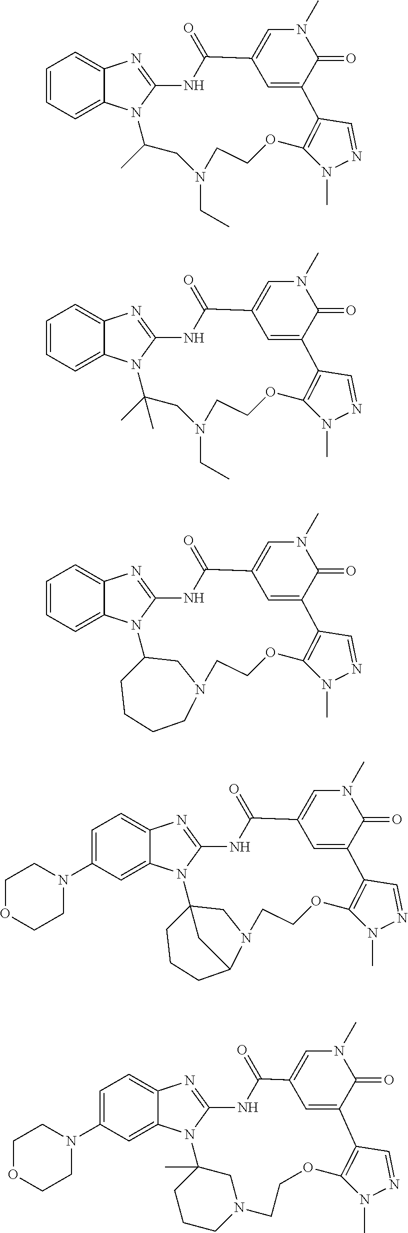 [SMILES: CCN1CCOc2c(cnn2C)-c2cc(cn(C)c2=O)C(=O)Nc2nc3ccccc3n2C(C)C1 ; CCN1CCOc2c(cnn2C)-c2cc(cn(C)c2=O)C(=O)Nc2nc3ccccc3n2C(C)(C)C1 ; Cn1ncc2c1OCCN1CCCCC(C1)n1c(nc3ccccc31)NC(=O)c1cc-2c(=O)n(C)c1 ; Cn1ncc2c1OCCN1CC3(CCCC1C3)n1c(nc3ccc(N4CCOCC4)cc31)NC(=O)c1cc-2c(=O)n(C)c1 ; Cn1ncc2c1OCCN1CCCC(C)(C1)n1c(nc3ccc(N4CCOCC4)cc31)NC(=O)c1cc-2c(=O)n(C)c1]

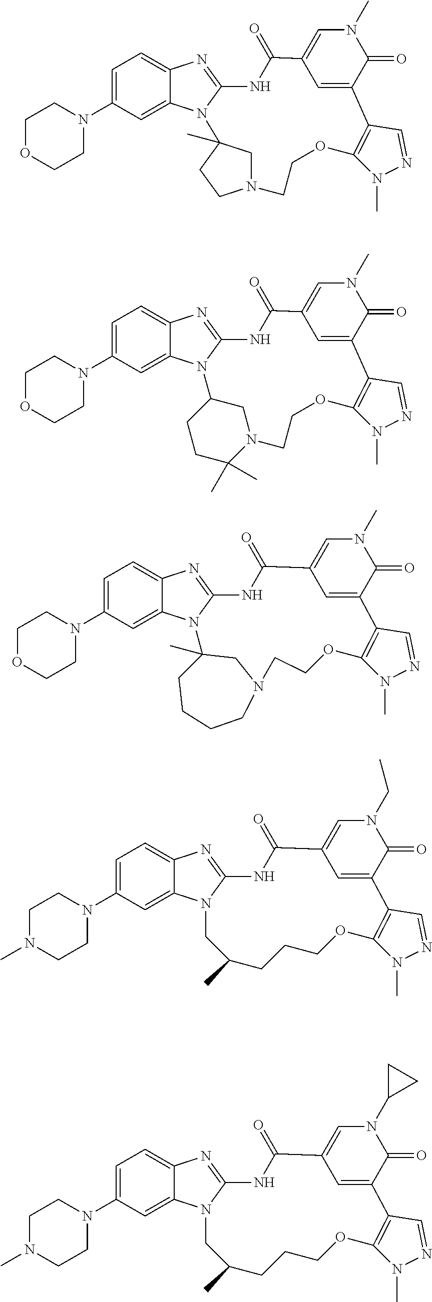

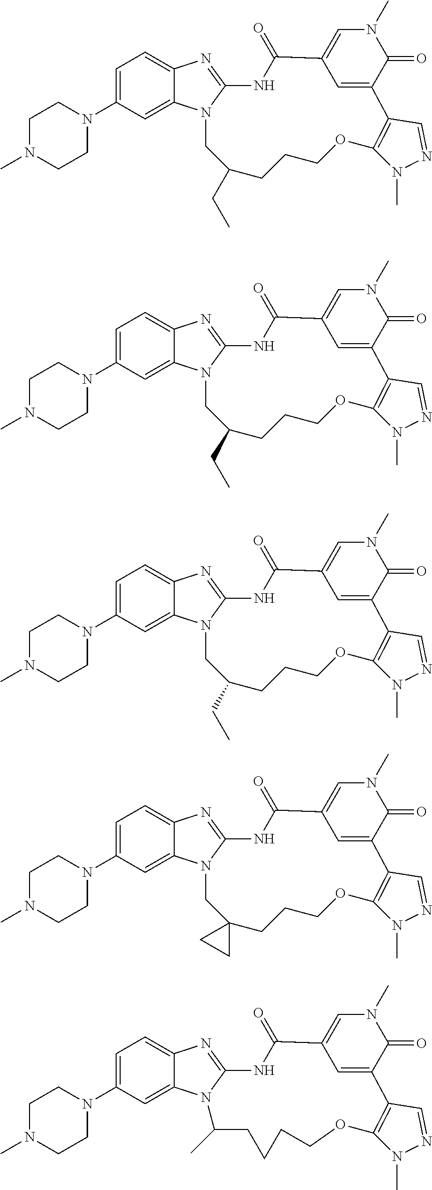

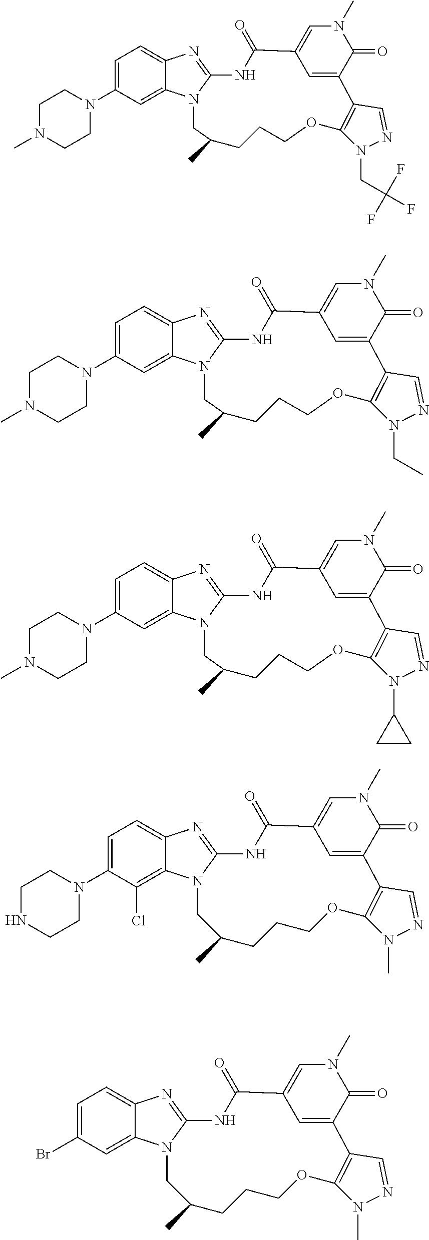

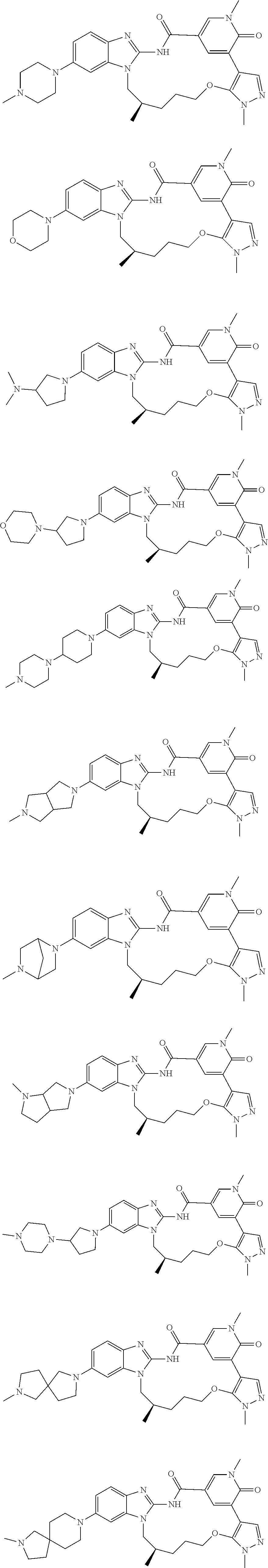

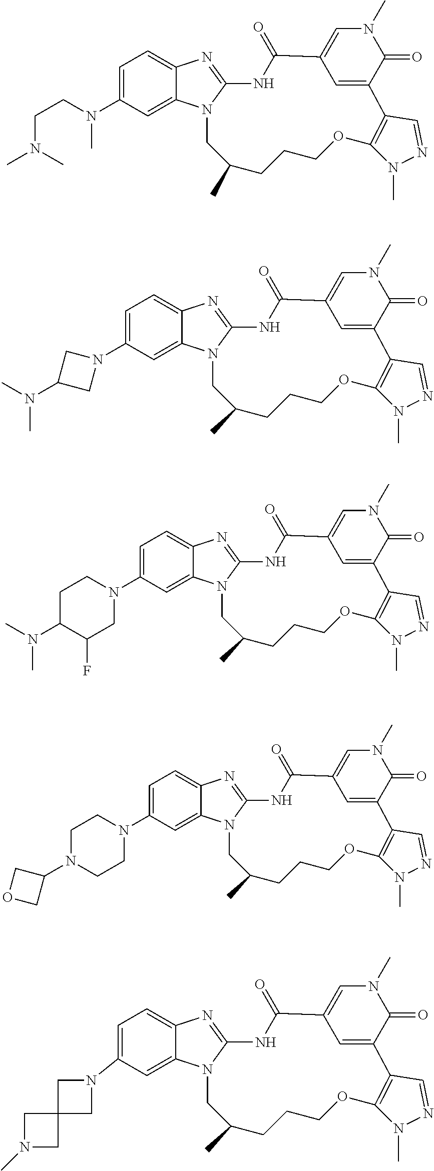

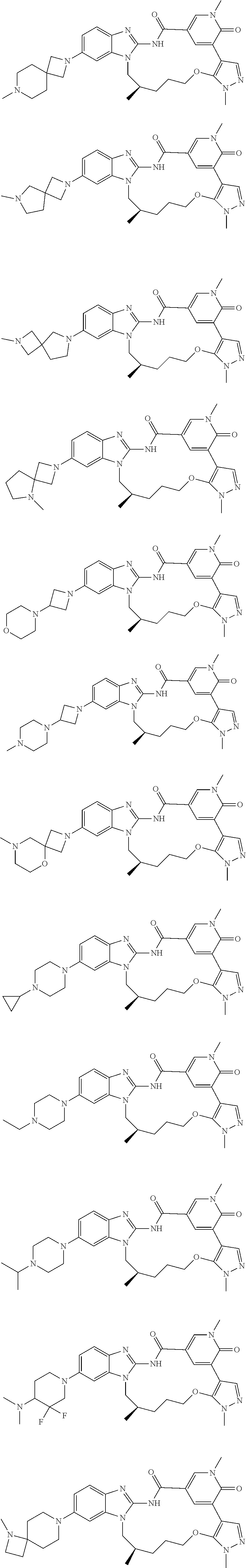

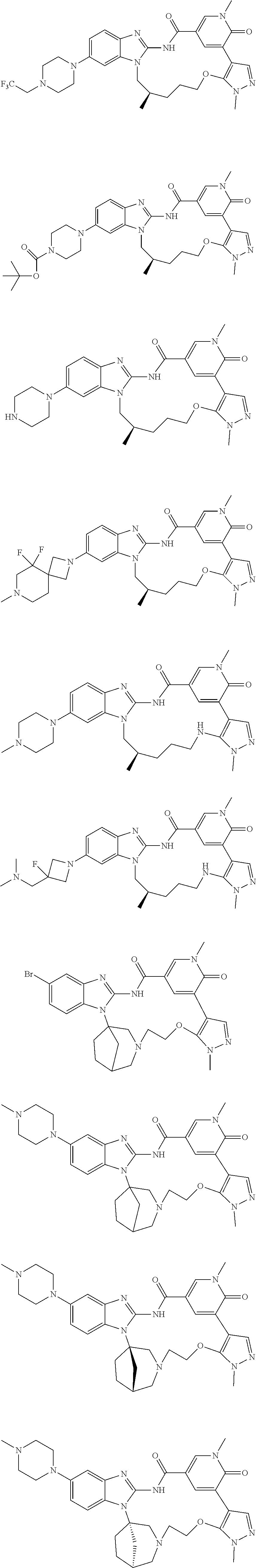

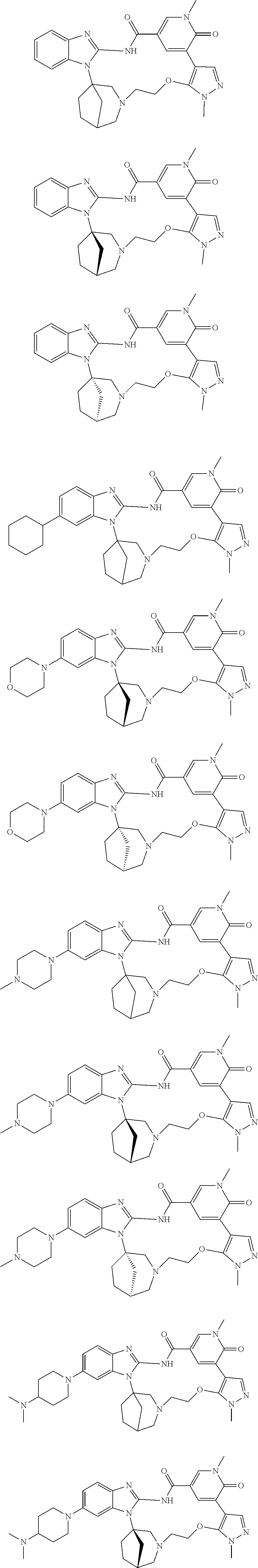

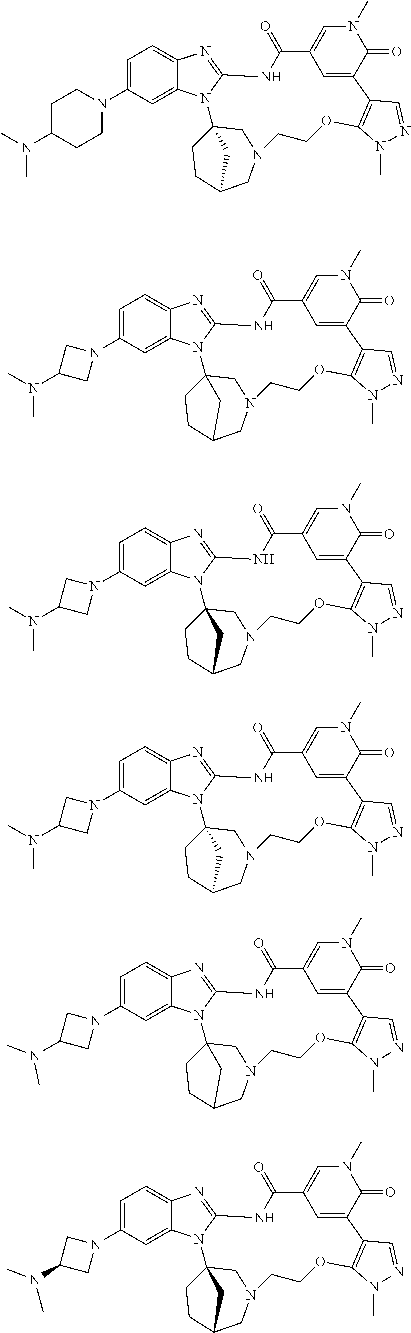

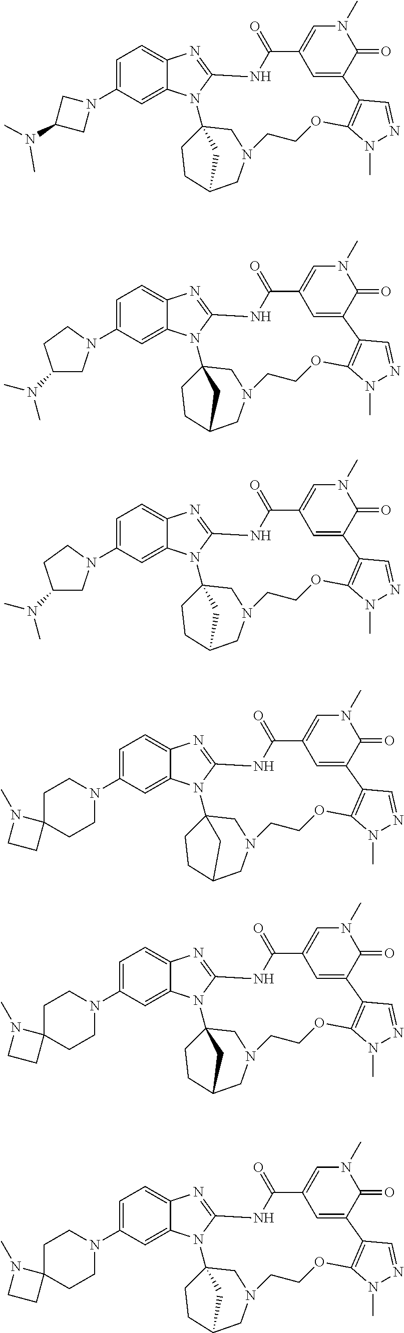

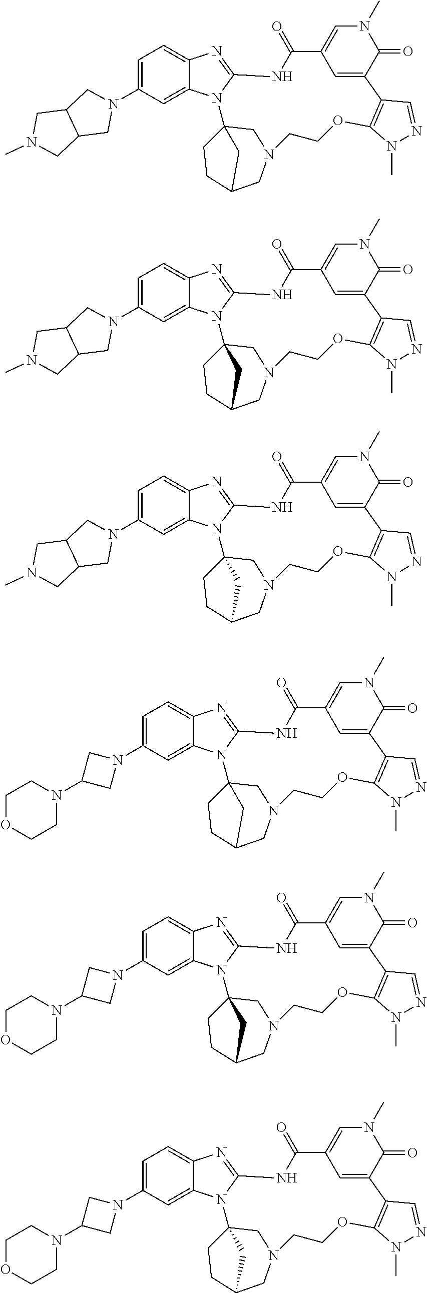

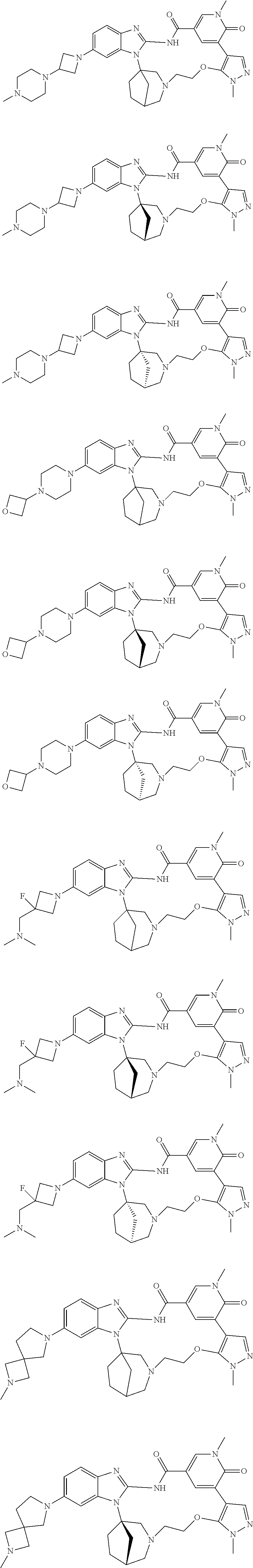

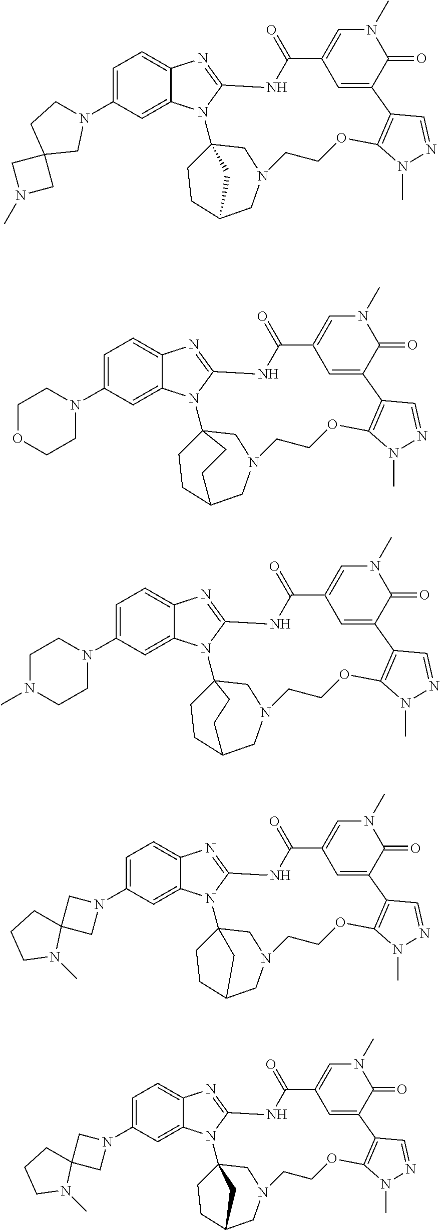 [SMILES: CN1CC2(CCN(c3ccc4nc5n(c4c3)[C@]34CC[C@H](CN(CCOc6c(cnn6C)-c6cc(cn(C)c6=O)C(=O)N5)C3)C4)C2)C1 ; Cn1ncc2c1OCCN1CC3CCC(CC3)(C1)n1c(nc3ccc(N4CCOCC4)cc31)NC(=O)c1cc-2c(=O)n(C)c1 ; CN1CCN(c2ccc3nc4n(c3c2)C23CCC(CC2)CN(CCOc2c(cnn2C)-c2cc(cn(C)c2=O)C(=O)N4)C3)CC1 ; CN1CCCC12CN(c1ccc3nc4n(c3c1)C13CCC(CN(CCOc5c(cnn5C)-c5cc(cn(C)c5=O)C(=O)N4)C1)C3)C2 ; CN1CCCC12CN(c1ccc3nc4n(c3c1)[C@@]13CC[C@@H](CN(CCOc5c(cnn5C)-c5cc(cn(C)c5=O)C(=O)N4)C1)C3)C2]

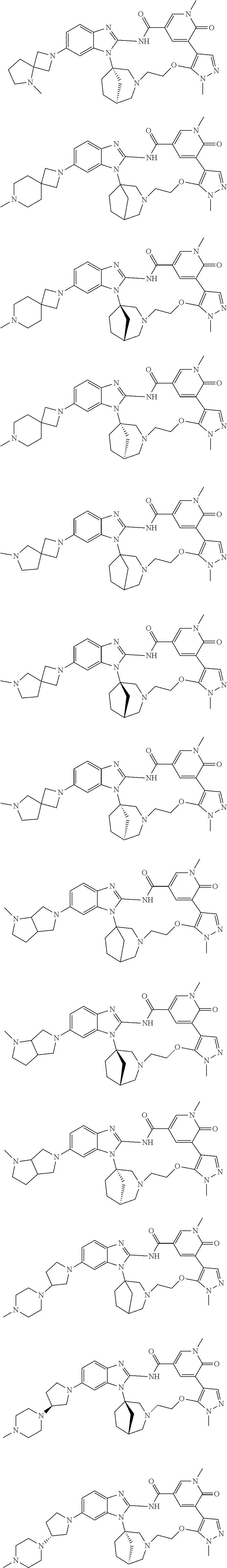

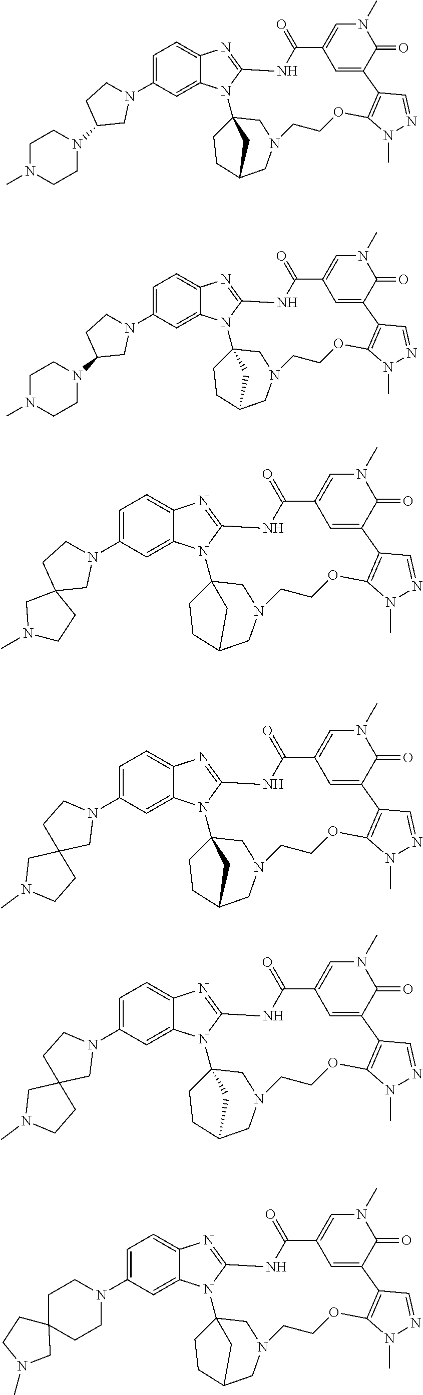

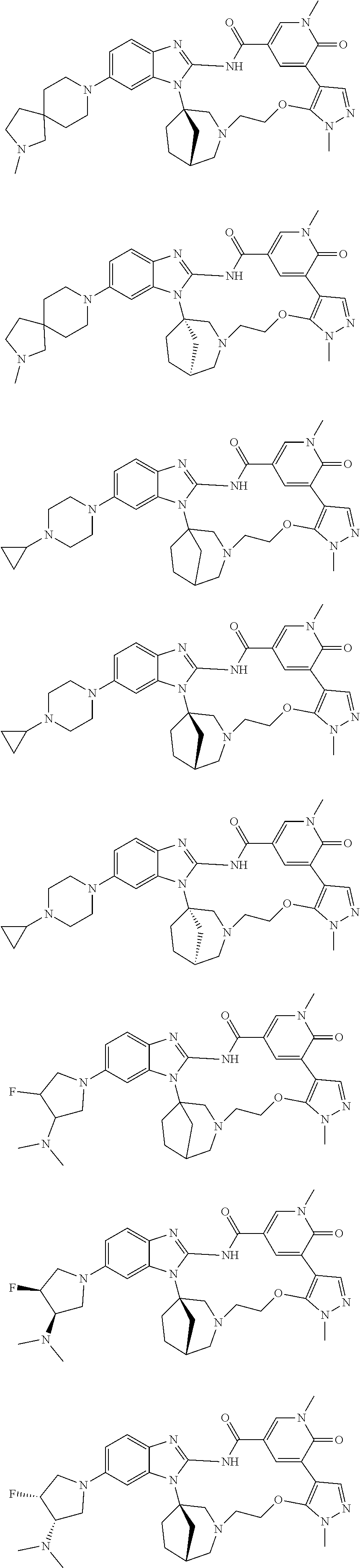

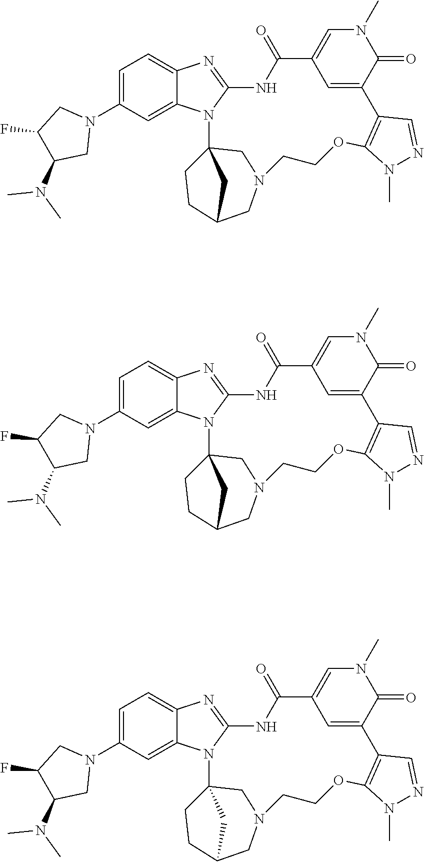 [SMILES: CN(C)[C@@H]1CN(c2ccc3nc4n(c3c2)[C@@]23CC[C@@H](CN(CCOc5c(cnn5C)-c5cc(cn(C)c5=O)C(=O)N4)C2)C3)C[C@H]1F ; CN(C)[C@H]1CN(c2ccc3nc4n(c3c2)[C@@]23CC[C@@H](CN(CCOc5c(cnn5C)-c5cc(cn(C)c5=O)C(=O)N4)C2)C3)C[C@@H]1F ; CN(C)[C@@H]1CN(c2ccc3nc4n(c3c2)[C@]23CC[C@H](CN(CCOc5c(cnn5C)-c5cc(cn(C)c5=O)C(=O)N4)C2)C3)C[C@@H]1F]

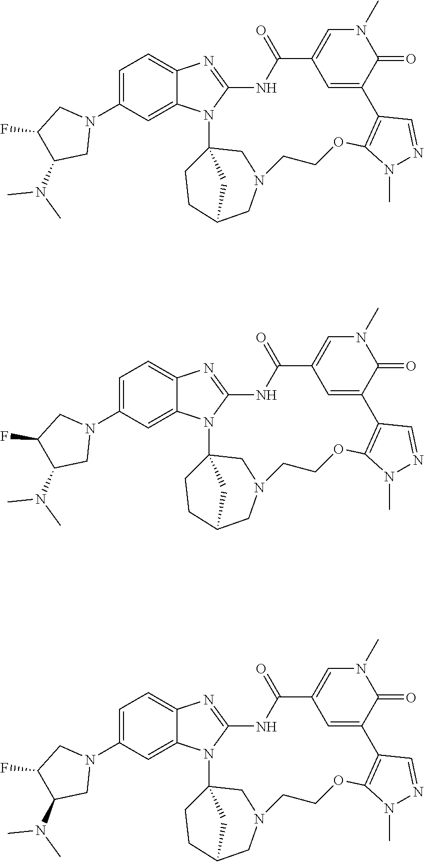 [SMILES: CN(C)[C@H]1CN(c2ccc3nc4n(c3c2)[C@]23CC[C@H](CN(CCOc5c(cnn5C)-c5cc(cn(C)c5=O)C(=O)N4)C2)C3)C[C@H]1F ; CN(C)[C@H]1CN(c2ccc3nc4n(c3c2)[C@]23CC[C@H](CN(CCOc5c(cnn5C)-c5cc(cn(C)c5=O)C(=O)N4)C2)C3)C[C@@H]1F ; CN(C)[C@@H]1CN(c2ccc3nc4n(c3c2)[C@]23CC[C@H](CN(CCOc5c(cnn5C)-c5cc(cn(C)c5=O)C(=O)N4)C2)C3)C[C@H]1F]

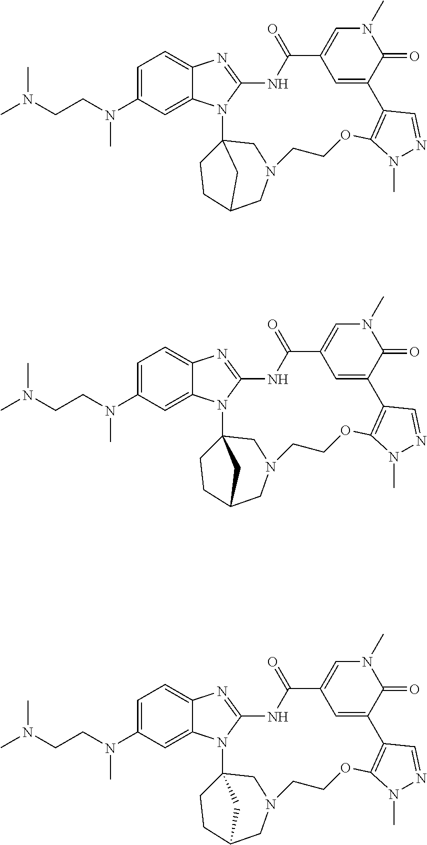 [SMILES: CN(C)CCN(C)c1ccc2nc3n(c2c1)C12CCC(CN(CCOc4c(cnn4C)-c4cc(cn(C)c4=O)C(=O)N3)C1)C2 ; CN(C)CCN(C)c1ccc2nc3n(c2c1)[C@@]12CC[C@@H](CN(CCOc4c(cnn4C)-c4cc(cn(C)c4=O)C(=O)N3)C1)C2 ; CN(C)CCN(C)c1ccc2nc3n(c2c1)[C@]12CC[C@H](CN(CCOc4c(cnn4C)-c4cc(cn(C)c4=O)C(=O)N3)C1)C2]

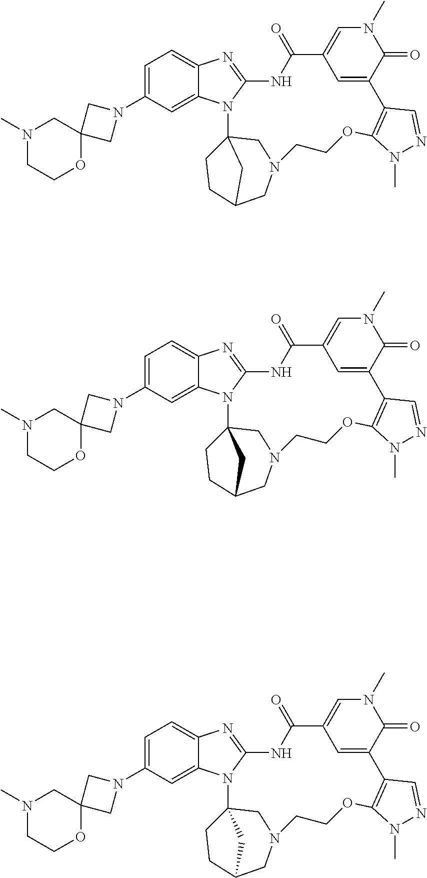 [SMILES: CN1CCOC2(C1)CN(c1ccc3nc4n(c3c1)C13CCC(CN(CCOc5c(cnn5C)-c5cc(cn(C)c5=O)C(=O)N4)C1)C3)C2 ; CN1CCOC2(C1)CN(c1ccc3nc4n(c3c1)[C@@]13CC[C@@H](CN(CCOc5c(cnn5C)-c5cc(cn(C)c5=O)C(=O)N4)C1)C3)C2 ; CN1CCOC2(C1)CN(c1ccc3nc4n(c3c1)[C@]13CC[C@H](CN(CCOc5c(cnn5C)-c5cc(cn(C)c5=O)C(=O)N4)C1)C3)C2]

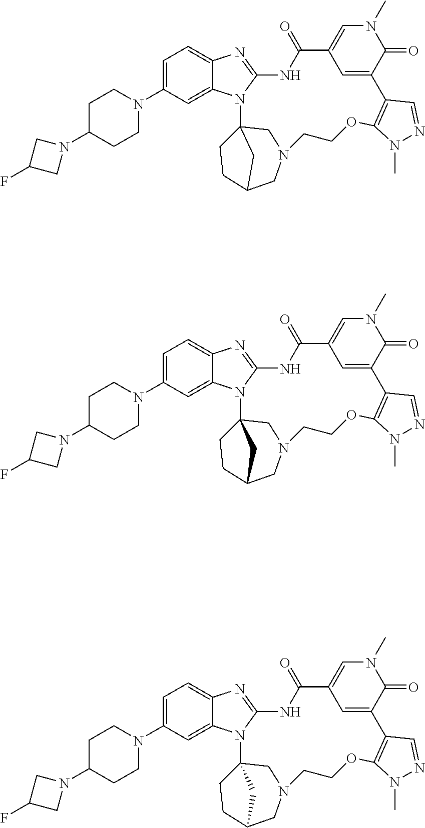 [SMILES: Cn1ncc2c1OCCN1CC3CCC(C3)(C1)n1c(nc3ccc(N4CCC(N5CC(F)C5)CC4)cc31)NC(=O)c1cc-2c(=O)n(C)c1 ; Cn1ncc2c1OCCN1C[C@@H]3CC[C@@](C3)(C1)n1c(nc3ccc(N4CCC(N5CC(F)C5)CC4)cc31)NC(=O)c1cc-2c(=O)n(C)c1 ; Cn1ncc2c1OCCN1C[C@H]3CC[C@](C3)(C1)n1c(nc3ccc(N4CCC(N5CC(F)C5)CC4)cc31)NC(=O)c1cc-2c(=O)n(C)c1]

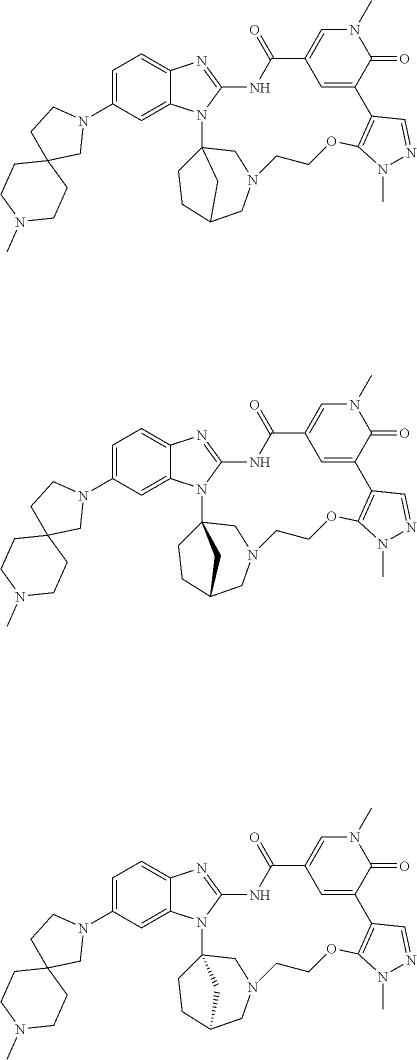 [SMILES: CN1CCC2(CC1)CCN(c1ccc3nc4n(c3c1)C13CCC(CN(CCOc5c(cnn5C)-c5cc(cn(C)c5=O)C(=O)N4)C1)C3)C2 ; CN1CCC2(CC1)CCN(c1ccc3nc4n(c3c1)[C@@]13CC[C@@H](CN(CCOc5c(cnn5C)-c5cc(cn(C)c5=O)C(=O)N4)C1)C3)C2 ; CN1CCC2(CC1)CCN(c1ccc3nc4n(c3c1)[C@]13CC[C@H](CN(CCOc5c(cnn5C)-c5cc(cn(C)c5=O)C(=O)N4)C1)C3)C2]

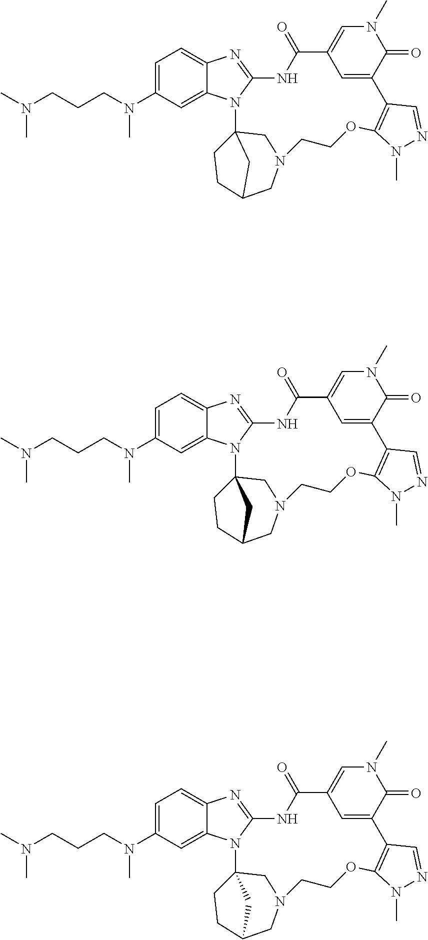 [SMILES: CN(C)CCCN(C)c1ccc2nc3n(c2c1)C12CCC(CN(CCOc4c(cnn4C)-c4cc(cn(C)c4=O)C(=O)N3)C1)C2 ; CN(C)CCCN(C)c1ccc2nc3n(c2c1)[C@@]12CC[C@@H](CN(CCOc4c(cnn4C)-c4cc(cn(C)c4=O)C(=O)N3)C1)C2 ; CN(C)CCCN(C)c1ccc2nc3n(c2c1)[C@]12CC[C@H](CN(CCOc4c(cnn4C)-c4cc(cn(C)c4=O)C(=O)N3)C1)C2]

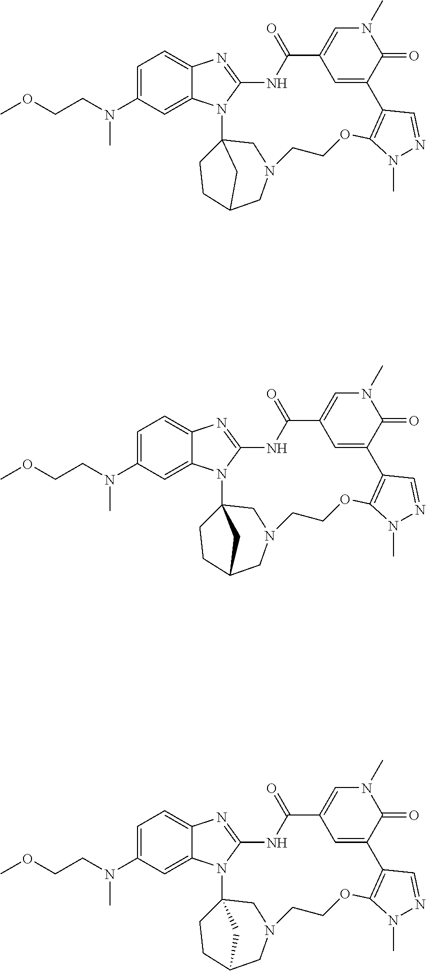 [SMILES: COCCN(C)c1ccc2nc3n(c2c1)C12CCC(CN(CCOc4c(cnn4C)-c4cc(cn(C)c4=O)C(=O)N3)C1)C2 ; COCCN(C)c1ccc2nc3n(c2c1)[C@@]12CC[C@@H](CN(CCOc4c(cnn4C)-c4cc(cn(C)c4=O)C(=O)N3)C1)C2 ; COCCN(C)c1ccc2nc3n(c2c1)[C@]12CC[C@H](CN(CCOc4c(cnn4C)-c4cc(cn(C)c4=O)C(=O)N3)C1)C2]

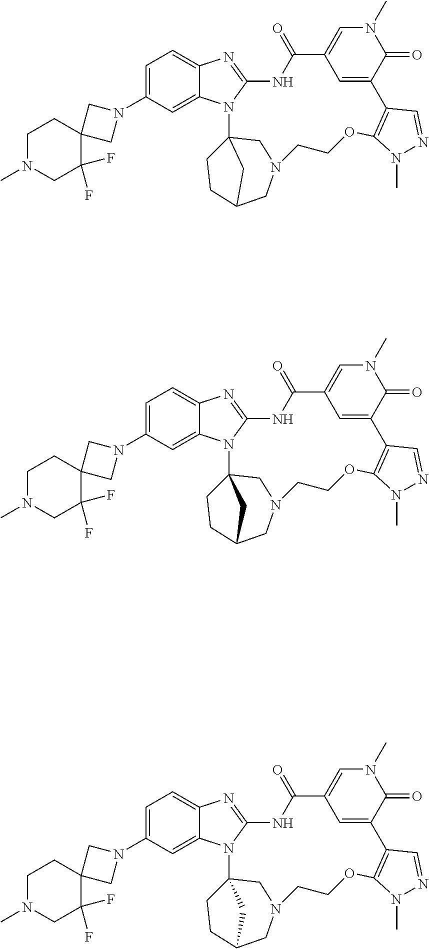 [SMILES: CN1CCC2(CN(c3ccc4nc5n(c4c3)C34CCC(CN(CCOc6c(cnn6C)-c6cc(cn(C)c6=O)C(=O)N5)C3)C4)C2)C(F)(F)C1 ; CN1CCC2(CN(c3ccc4nc5n(c4c3)[C@@]34CC[C@@H](CN(CCOc6c(cnn6C)-c6cc(cn(C)c6=O)C(=O)N5)C3)C4)C2)C(F)(F)C1 ; CN1CCC2(CN(c3ccc4nc5n(c4c3)[C@]34CC[C@H](CN(CCOc6c(cnn6C)-c6cc(cn(C)c6=O)C(=O)N5)C3)C4)C2)C(F)(F)C1]

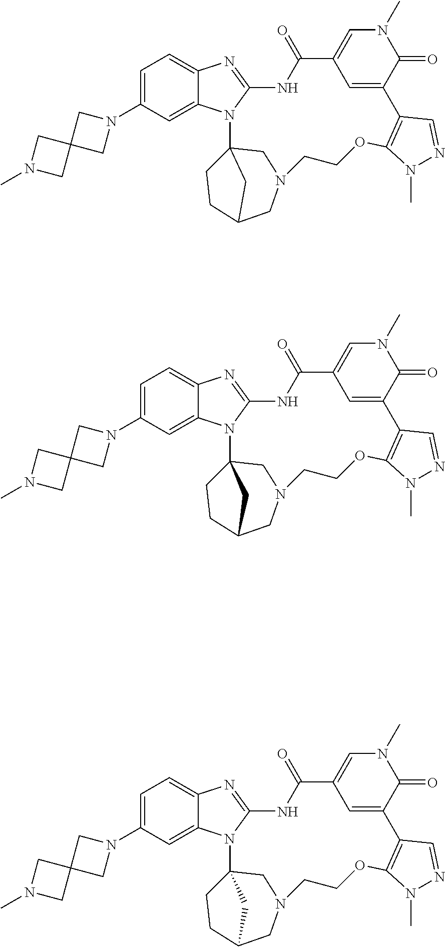 [SMILES: CN1CC2(C1)CN(c1ccc3nc4n(c3c1)C13CCC(CN(CCOc5c(cnn5C)-c5cc(cn(C)c5=O)C(=O)N4)C1)C3)C2 ; CN1CC2(C1)CN(c1ccc3nc4n(c3c1)[C@@]13CC[C@@H](CN(CCOc5c(cnn5C)-c5cc(cn(C)c5=O)C(=O)N4)C1)C3)C2 ; CN1CC2(C1)CN(c1ccc3nc4n(c3c1)[C@]13CC[C@H](CN(CCOc5c(cnn5C)-c5cc(cn(C)c5=O)C(=O)N4)C1)C3)C2]

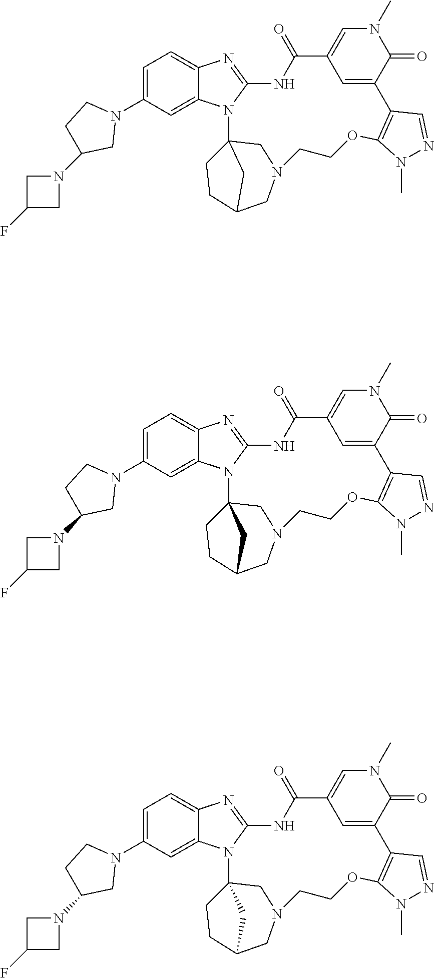 [SMILES: Cn1ncc2c1OCCN1CC3CCC(C3)(C1)n1c(nc3ccc(N4CCC(N5CC(F)C5)C4)cc31)NC(=O)c1cc-2c(=O)n(C)c1 ; Cn1ncc2c1OCCN1C[C@@H]3CC[C@@](C3)(C1)n1c(nc3ccc(N4CC[C@H](N5CC(F)C5)C4)cc31)NC(=O)c1cc-2c(=O)n(C)c1 ; Cn1ncc2c1OCCN1C[C@H]3CC[C@](C3)(C1)n1c(nc3ccc(N4CC[C@@H](N5CC(F)C5)C4)cc31)NC(=O)c1cc-2c(=O)n(C)c1]

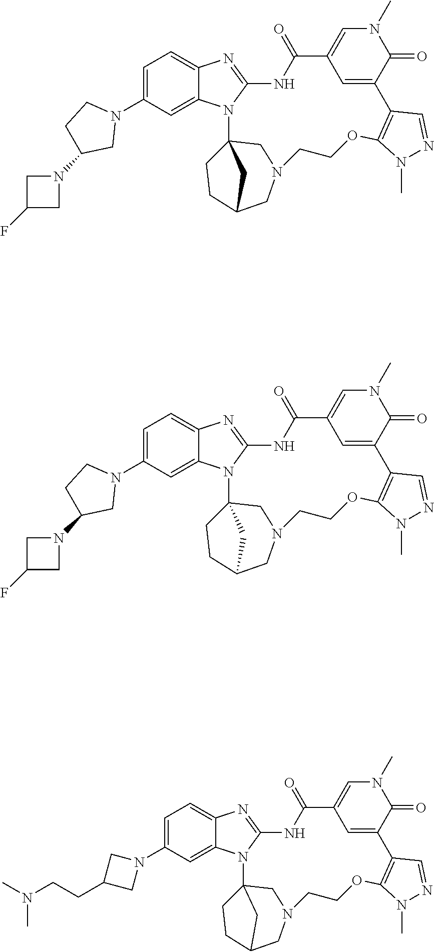 [SMILES: Cn1ncc2c1OCCN1C[C@@H]3CC[C@@](C3)(C1)n1c(nc3ccc(N4CC[C@@H](N5CC(F)C5)C4)cc31)NC(=O)c1cc-2c(=O)n(C)c1 ; Cn1ncc2c1OCCN1C[C@H]3CC[C@](C3)(C1)n1c(nc3ccc(N4CC[C@H](N5CC(F)C5)C4)cc31)NC(=O)c1cc-2c(=O)n(C)c1 ; CN(C)CCC1CN(c2ccc3nc4n(c3c2)C23CCC(CN(CCOc5c(cnn5C)-c5cc(cn(C)c5=O)C(=O)N4)C2)C3)C1]

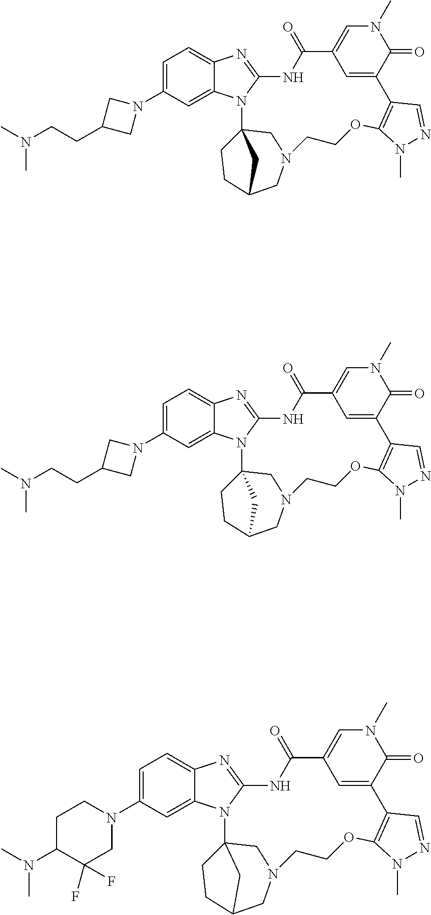 [SMILES: CN(C)CCC1CN(c2ccc3nc4n(c3c2)[C@@]23CC[C@@H](CN(CCOc5c(cnn5C)-c5cc(cn(C)c5=O)C(=O)N4)C2)C3)C1 ; CN(C)CCC1CN(c2ccc3nc4n(c3c2)[C@]23CC[C@H](CN(CCOc5c(cnn5C)-c5cc(cn(C)c5=O)C(=O)N4)C2)C3)C1 ; CN(C)C1CCN(c2ccc3nc4n(c3c2)C23CCC(CN(CCOc5c(cnn5C)-c5cc(cn(C)c5=O)C(=O)N4)C2)C3)CC1(F)F]

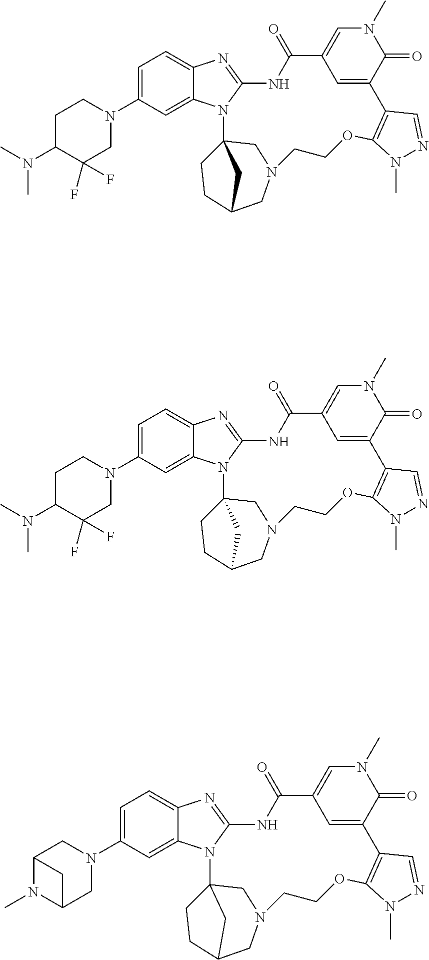 [SMILES: CN(C)C1CCN(c2ccc3nc4n(c3c2)[C@@]23CC[C@@H](CN(CCOc5c(cnn5C)-c5cc(cn(C)c5=O)C(=O)N4)C2)C3)CC1(F)F ; CN(C)C1CCN(c2ccc3nc4n(c3c2)[C@]23CC[C@H](CN(CCOc5c(cnn5C)-c5cc(cn(C)c5=O)C(=O)N4)C2)C3)CC1(F)F ; CN1C2CC1CN(c1ccc3nc4n(c3c1)C13CCC(CN(CCOc5c(cnn5C)-c5cc(cn(C)c5=O)C(=O)N4)C1)C3)C2]

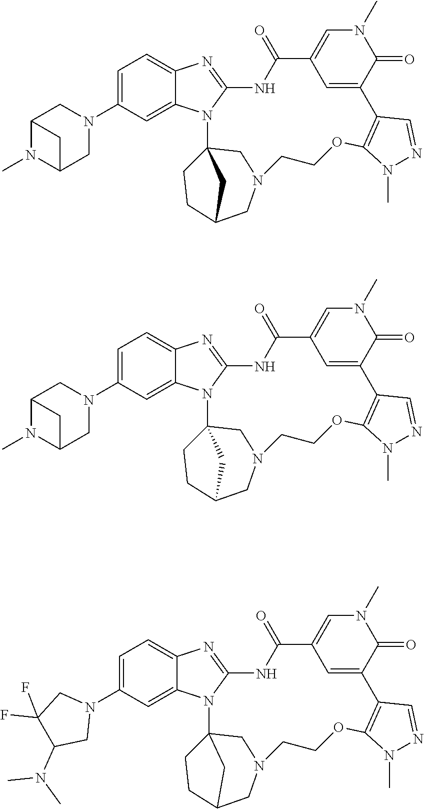 [SMILES: CN1C2CC1CN(c1ccc3nc4n(c3c1)[C@@]13CC[C@@H](CN(CCOc5c(cnn5C)-c5cc(cn(C)c5=O)C(=O)N4)C1)C3)C2 ; CN1C2CC1CN(c1ccc3nc4n(c3c1)[C@]13CC[C@H](CN(CCOc5c(cnn5C)-c5cc(cn(C)c5=O)C(=O)N4)C1)C3)C2 ; CN(C)C1CN(c2ccc3nc4n(c3c2)C23CCC(CN(CCOc5c(cnn5C)-c5cc(cn(C)c5=O)C(=O)N4)C2)C3)CC1(F)F]

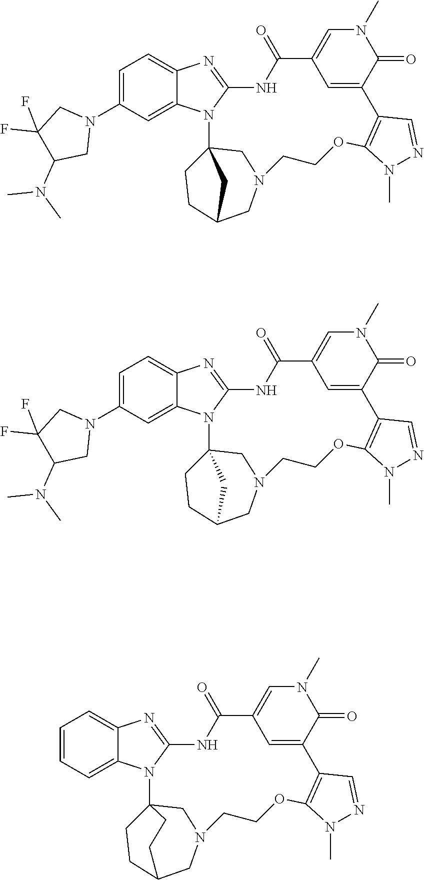 [SMILES: CN(C)C1CN(c2ccc3nc4n(c3c2)[C@@]23CC[C@@H](CN(CCOc5c(cnn5C)-c5cc(cn(C)c5=O)C(=O)N4)C2)C3)CC1(F)F ; CN(C)C1CN(c2ccc3nc4n(c3c2)[C@]23CC[C@H](CN(CCOc5c(cnn5C)-c5cc(cn(C)c5=O)C(=O)N4)C2)C3)CC1(F)F ; Cn1ncc2c1OCCN1CC3CCC(CC3)(C1)n1c(nc3ccccc31)NC(=O)c1cc-2c(=O)n(C)c1]

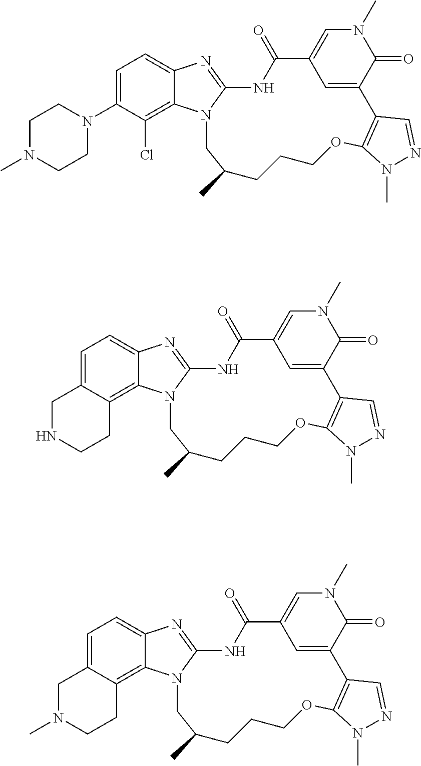 [SMILES: C[C@@H]1CCCOc2c(cnn2C)-c2cc(cn(C)c2=O)C(=O)Nc2nc3ccc(N4CCN(C)CC4)c(Cl)c3n2C1 ; C[C@@H]1CCCOc2c(cnn2C)-c2cc(cn(C)c2=O)C(=O)Nc2nc3ccc4c(c3n2C1)CCNC4 ; C[C@@H]1CCCOc2c(cnn2C)-c2cc(cn(C)c2=O)C(=O)Nc2nc3ccc4c(c3n2C1)CCN(C)C4]

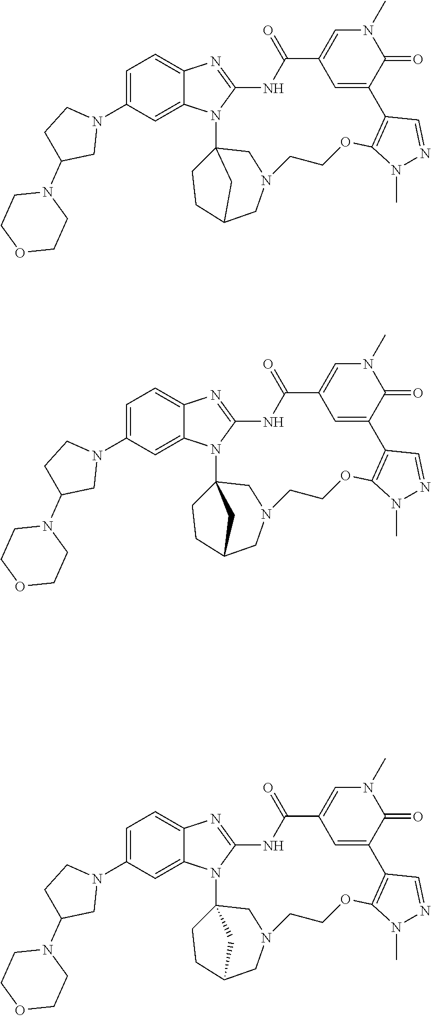 [SMILES: Cn1ncc2c1OCCN1CC3CCC(C3)(C1)n1c(nc3ccc(N4CCC(N5CCOCC5)C4)cc31)NC(=O)c1cc-2c(=O)n(C)c1 ; Cn1ncc2c1OCCN1C[C@@H]3CC[C@@](C3)(C1)n1c(nc3ccc(N4CCC(N5CCOCC5)C4)cc31)NC(=O)c1cc-2c(=O)n(C)c1 ; Cn1ncc2c1OCCN1C[C@H]3CC[C@](C3)(C1)n1c(nc3ccc(N4CCC(N5CCOCC5)C4)cc31)NC(=O)c1cc-2c(=O)n(C)c1]

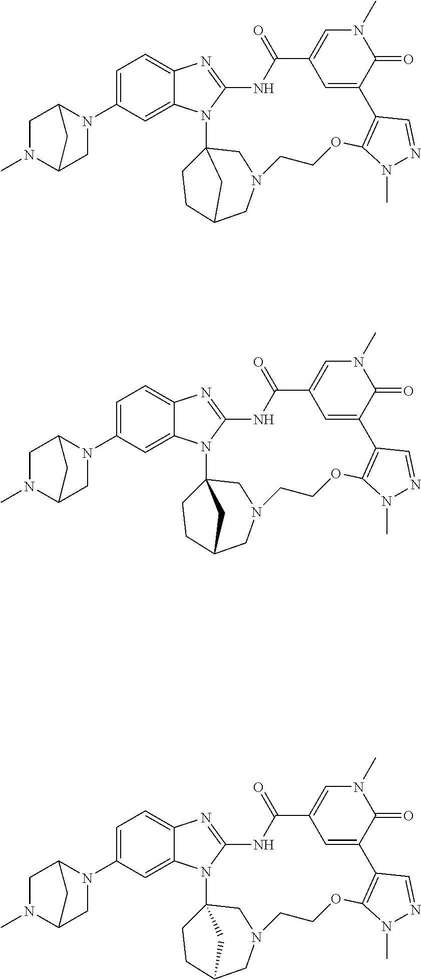 [SMILES: CN1CC2CC1CN2c1ccc2nc3n(c2c1)C12CCC(CN(CCOc4c(cnn4C)-c4cc(cn(C)c4=O)C(=O)N3)C1)C2 ; CN1CC2CC1CN2c1ccc2nc3n(c2c1)[C@@]12CC[C@@H](CN(CCOc4c(cnn4C)-c4cc(cn(C)c4=O)C(=O)N3)C1)C2 ; CN1CC2CC1CN2c1ccc2nc3n(c2c1)[C@]12CC[C@H](CN(CCOc4c(cnn4C)-c4cc(cn(C)c4=O)C(=O)N3)C1)C2]

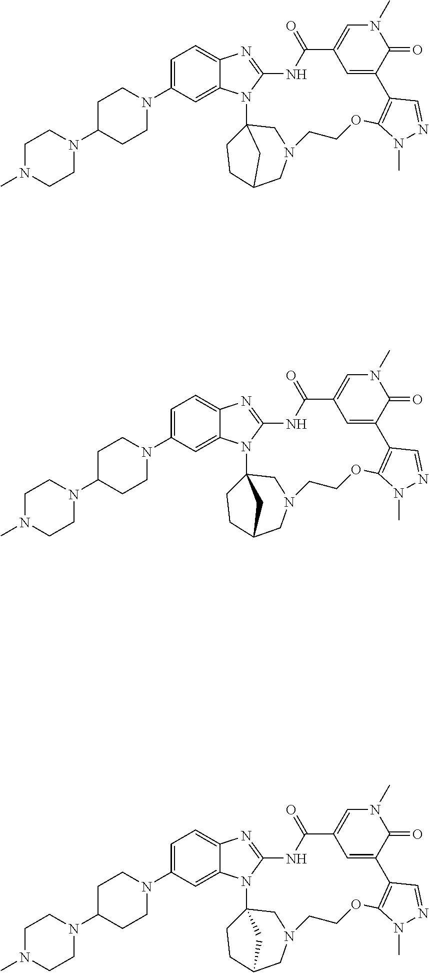 [SMILES: CN1CCN(C2CCN(c3ccc4nc5n(c4c3)C34CCC(CN(CCOc6c(cnn6C)-c6cc(cn(C)c6=O)C(=O)N5)C3)C4)CC2)CC1 ; CN1CCN(C2CCN(c3ccc4nc5n(c4c3)[C@@]34CC[C@@H](CN(CCOc6c(cnn6C)-c6cc(cn(C)c6=O)C(=O)N5)C3)C4)CC2)CC1 ; CN1CCN(C2CCN(c3ccc4nc5n(c4c3)[C@]34CC[C@H](CN(CCOc6c(cnn6C)-c6cc(cn(C)c6=O)C(=O)N5)C3)C4)CC2)CC1]

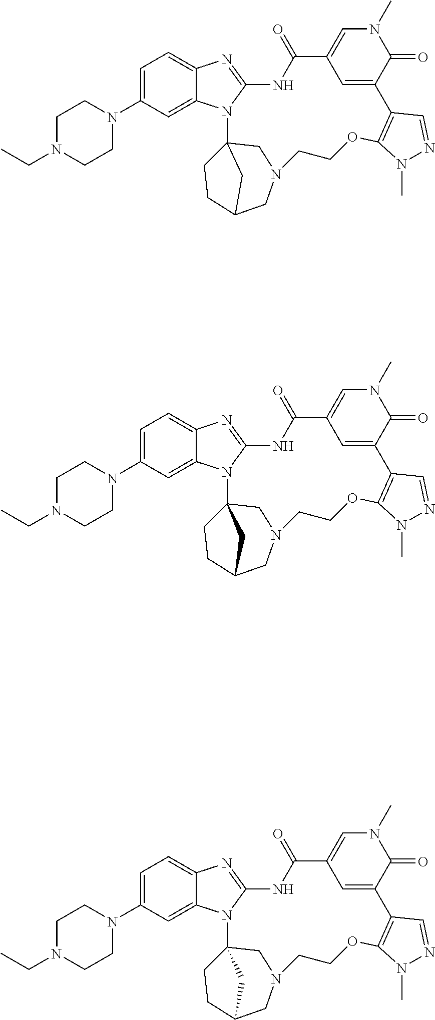 [SMILES: CCN1CCN(c2ccc3nc4n(c3c2)C23CCC(CN(CCOc5c(cnn5C)-c5cc(cn(C)c5=O)C(=O)N4)C2)C3)CC1 ; CCN1CCN(c2ccc3nc4n(c3c2)[C@@]23CC[C@@H](CN(CCOc5c(cnn5C)-c5cc(cn(C)c5=O)C(=O)N4)C2)C3)CC1 ; CCN1CCN(c2ccc3nc4n(c3c2)[C@]23CC[C@H](CN(CCOc5c(cnn5C)-c5cc(cn(C)c5=O)C(=O)N4)C2)C3)CC1]

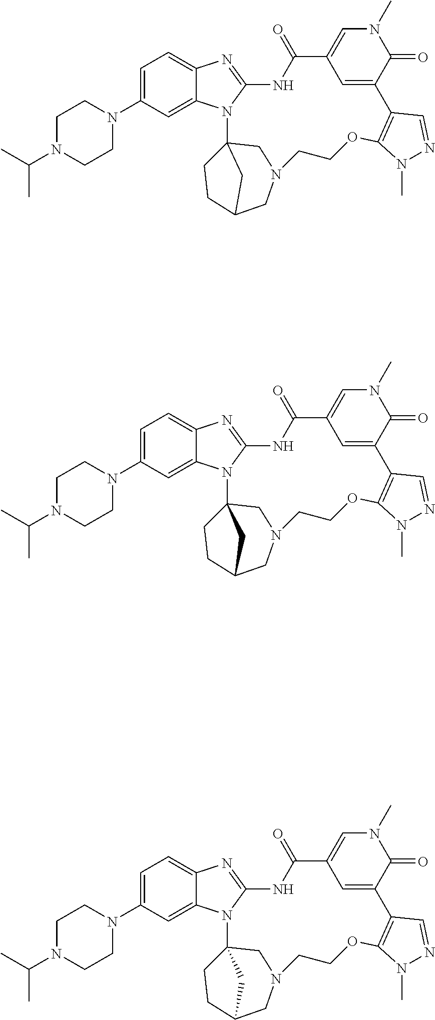 [SMILES: CC(C)N1CCN(c2ccc3nc4n(c3c2)C23CCC(CN(CCOc5c(cnn5C)-c5cc(cn(C)c5=O)C(=O)N4)C2)C3)CC1 ; CC(C)N1CCN(c2ccc3nc4n(c3c2)[C@@]23CC[C@@H](CN(CCOc5c(cnn5C)-c5cc(cn(C)c5=O)C(=O)N4)C2)C3)CC1 ; CC(C)N1CCN(c2ccc3nc4n(c3c2)[C@]23CC[C@H](CN(CCOc5c(cnn5C)-c5cc(cn(C)c5=O)C(=O)N4)C2)C3)CC1]

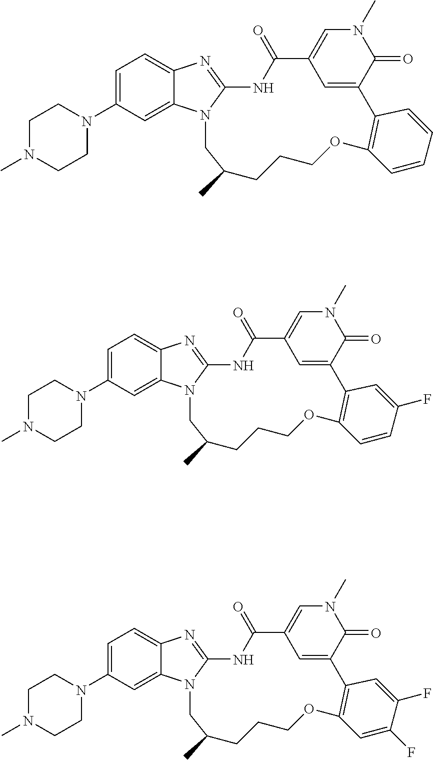 [SMILES: C[C@@H]1CCCOc2ccccc2-c2cc(cn(C)c2=O)C(=O)Nc2nc3ccc(N4CCN(C)CC4)cc3n2C1 ; C[C@@H]1CCCOc2ccc(F)cc2-c2cc(cn(C)c2=O)C(=O)Nc2nc3ccc(N4CCN(C)CC4)cc3n2C1 ; C[C@@H]1CCCOc2cc(F)c(F)cc2-c2cc(cn(C)c2=O)C(=O)Nc2nc3ccc(N4CCN(C)CC4)cc3n2C1]

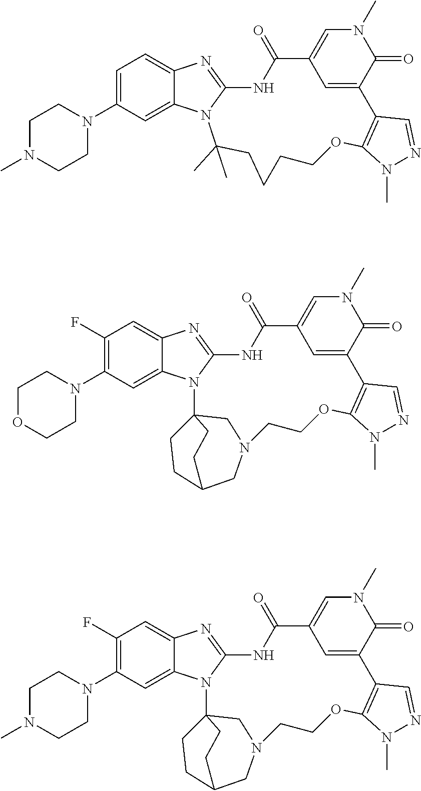 [SMILES: CN1CCN(c2ccc3nc4n(c3c2)C(C)(C)CCCCOc2c(cnn2C)-c2cc(cn(C)c2=O)C(=O)N4)CC1 ; Cn1ncc2c1OCCN1CC3CCC(CC3)(C1)n1c(nc3cc(F)c(N4CCOCC4)cc31)NC(=O)c1cc-2c(=O)n(C)c1 ; CN1CCN(c2cc3c(cc2F)nc2n3C34CCC(CC3)CN(CCOc3c(cnn3C)-c3cc(cn(C)c3=O)C(=O)N2)C4)CC1]

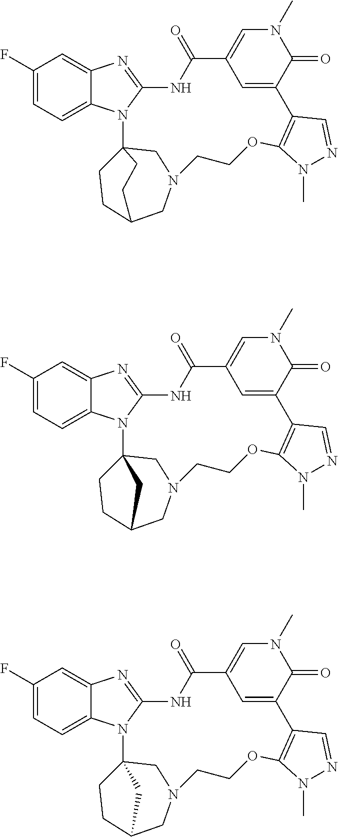 [SMILES: Cn1ncc2c1OCCN1CC3CCC(CC3)(C1)n1c(nc3cc(F)ccc31)NC(=O)c1cc-2c(=O)n(C)c1 ; Cn1ncc2c1OCCN1C[C@@H]3CC[C@@](C3)(C1)n1c(nc3cc(F)ccc31)NC(=O)c1cc-2c(=O)n(C)c1 ; Cn1ncc2c1OCCN1C[C@H]3CC[C@](C3)(C1)n1c(nc3cc(F)ccc31)NC(=O)c1cc-2c(=O)n(C)c1]

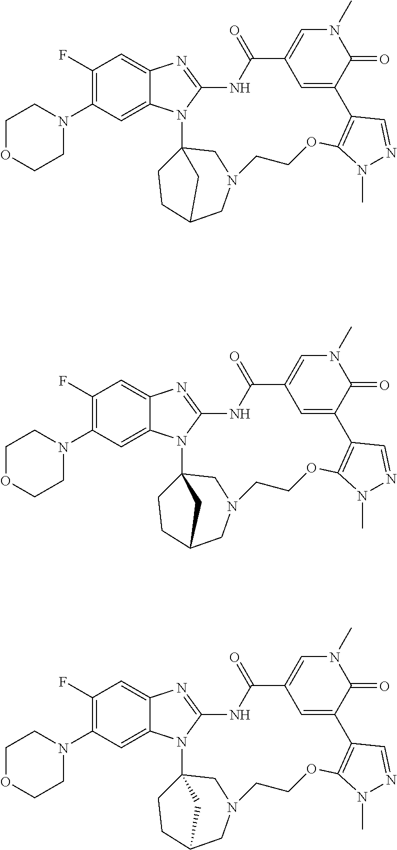 [SMILES: Cn1ncc2c1OCCN1CC3CCC(C3)(C1)n1c(nc3cc(F)c(N4CCOCC4)cc31)NC(=O)c1cc-2c(=O)n(C)c1 ; Cn1ncc2c1OCCN1C[C@@H]3CC[C@@](C3)(C1)n1c(nc3cc(F)c(N4CCOCC4)cc31)NC(=O)c1cc-2c(=O)n(C)c1 ; Cn1ncc2c1OCCN1C[C@H]3CC[C@](C3)(C1)n1c(nc3cc(F)c(N4CCOCC4)cc31)NC(=O)c1cc-2c(=O)n(C)c1]

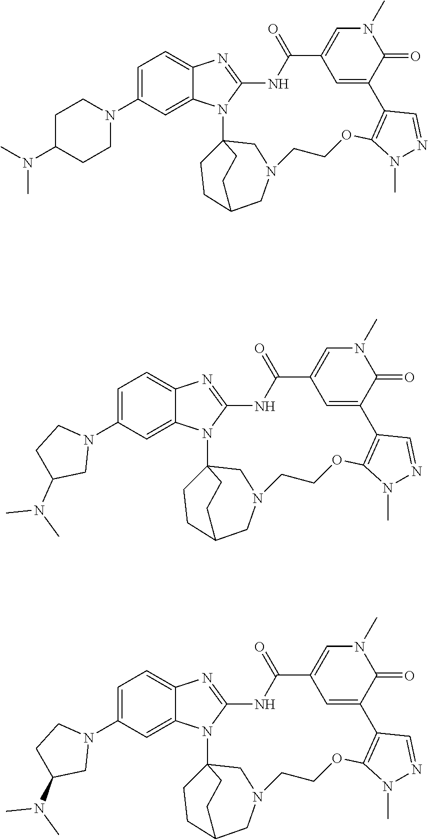 [SMILES: CN(C)C1CCN(c2ccc3nc4n(c3c2)C23CCC(CC2)CN(CCOc2c(cnn2C)-c2cc(cn(C)c2=O)C(=O)N4)C3)CC1 ; CN(C)C1CCN(c2ccc3nc4n(c3c2)C23CCC(CC2)CN(CCOc2c(cnn2C)-c2cc(cn(C)c2=O)C(=O)N4)C3)C1 ; CN(C)[C@H]1CCN(c2ccc3nc4n(c3c2)C23CCC(CC2)CN(CCOc2c(cnn2C)-c2cc(cn(C)c2=O)C(=O)N4)C3)C1]

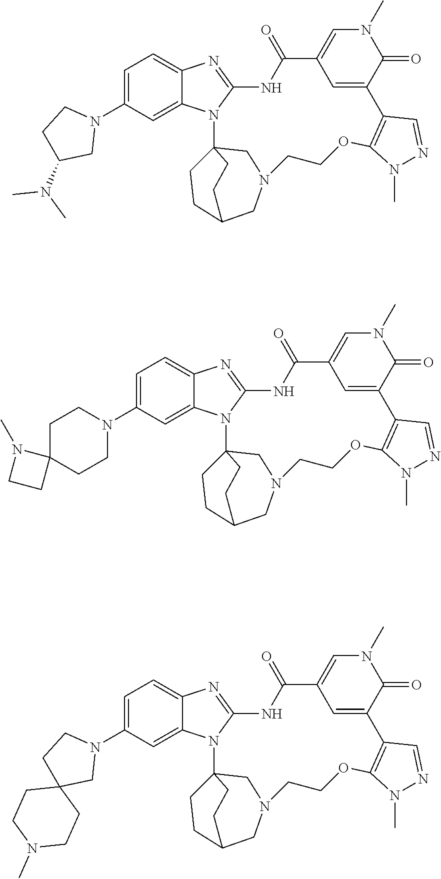 [SMILES: CN(C)[C@@H]1CCN(c2ccc3nc4n(c3c2)C23CCC(CC2)CN(CCOc2c(cnn2C)-c2cc(cn(C)c2=O)C(=O)N4)C3)C1 ; CN1CCC12CCN(c1ccc3nc4n(c3c1)C13CCC(CC1)CN(CCOc1c(cnn1C)-c1cc(cn(C)c1=O)C(=O)N4)C3)CC2 ; CN1CCC2(CC1)CCN(c1ccc3nc4n(c3c1)C13CCC(CC1)CN(CCOc1c(cnn1C)-c1cc(cn(C)c1=O)C(=O)N4)C3)C2]

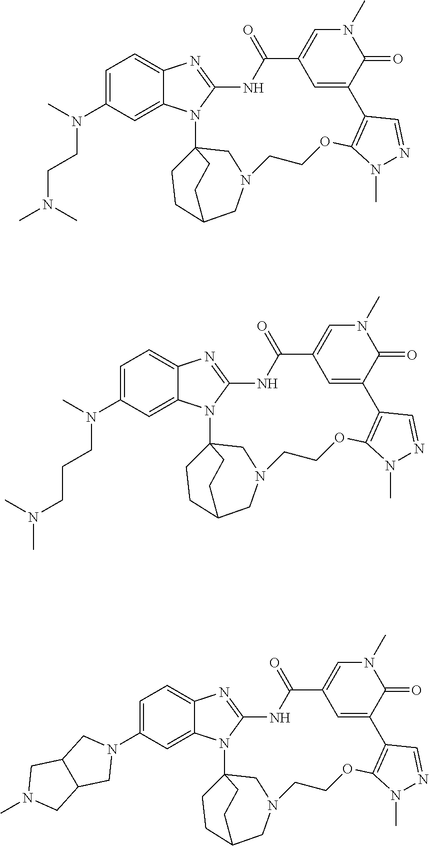 [SMILES: CN(C)CCN(C)c1ccc2nc3n(c2c1)C12CCC(CC1)CN(CCOc1c(cnn1C)-c1cc(cn(C)c1=O)C(=O)N3)C2 ; CN(C)CCCN(C)c1ccc2nc3n(c2c1)C12CCC(CC1)CN(CCOc1c(cnn1C)-c1cc(cn(C)c1=O)C(=O)N3)C2 ; CN1CC2CN(c3ccc4nc5n(c4c3)C34CCC(CC3)CN(CCOc3c(cnn3C)-c3cc(cn(C)c3=O)C(=O)N5)C4)CC2C1]

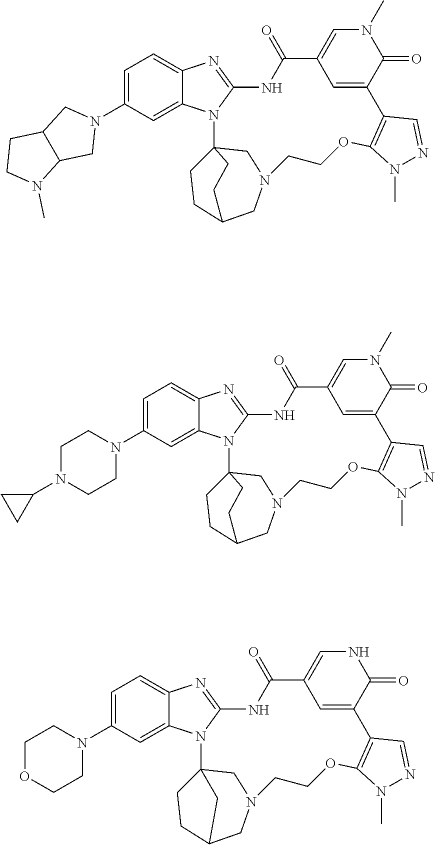 [SMILES: CN1CCC2CN(c3ccc4nc5n(c4c3)C34CCC(CC3)CN(CCOc3c(cnn3C)-c3cc(cn(C)c3=O)C(=O)N5)C4)CC21 ; Cn1ncc2c1OCCN1CC3CCC(CC3)(C1)n1c(nc3ccc(N4CCN(C5CC5)CC4)cc31)NC(=O)c1cc-2c(=O)n(C)c1 ; Cn1ncc2c1OCCN1CC3CCC(C3)(C1)n1c(nc3ccc(N4CCOCC4)cc31)NC(=O)c1c[nH]c(=O)c-2c1]

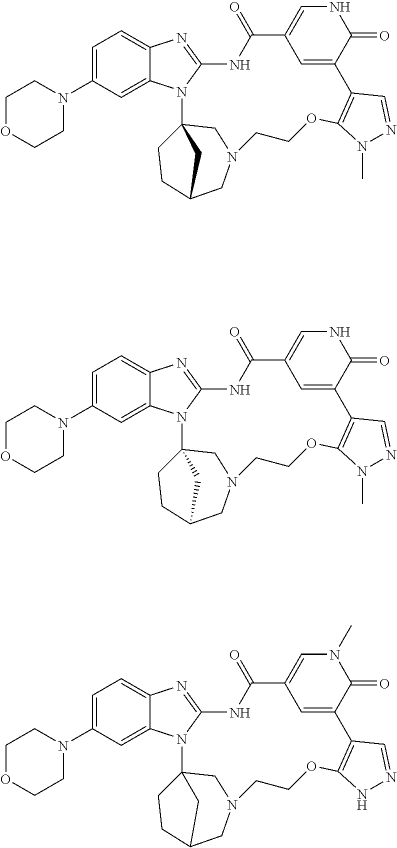

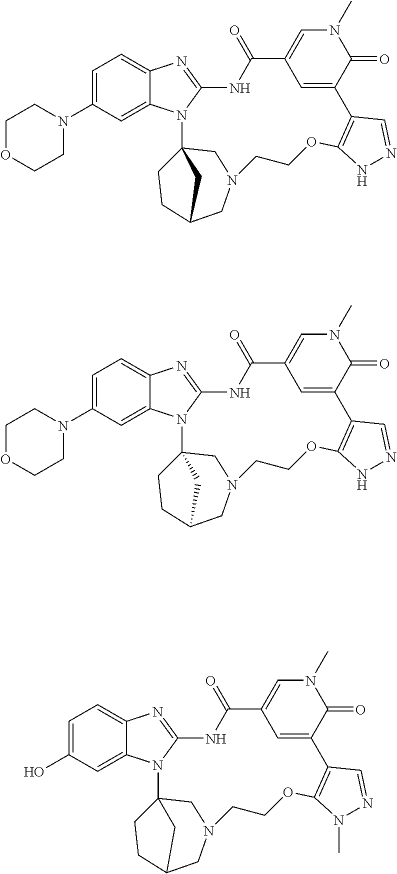

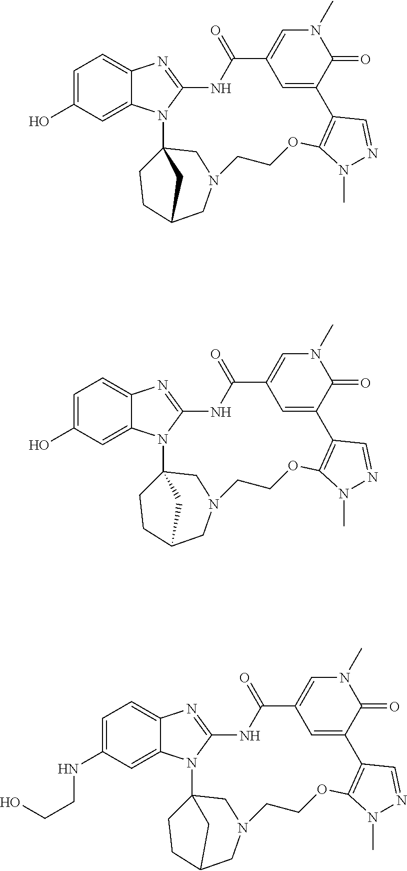 [SMILES: Cn1ncc2c1OCCN1C[C@@H]3CC[C@@](C3)(C1)n1c(nc3ccc(O)cc31)NC(=O)c1cc-2c(=O)n(C)c1 ; Cn1ncc2c1OCCN1C[C@H]3CC[C@](C3)(C1)n1c(nc3ccc(O)cc31)NC(=O)c1cc-2c(=O)n(C)c1 ; Cn1ncc2c1OCCN1CC3CCC(C3)(C1)n1c(nc3ccc(NCCO)cc31)NC(=O)c1cc-2c(=O)n(C)c1]

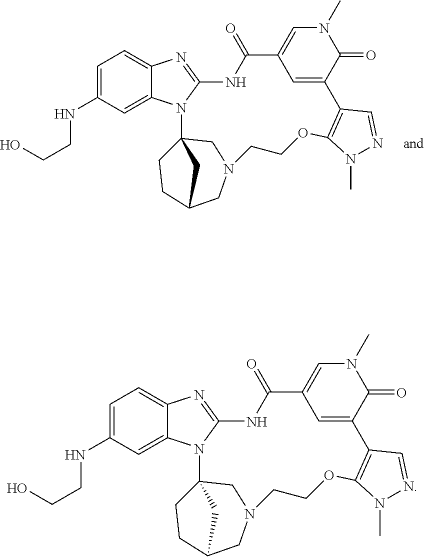

Embodiment 51. A pharmaceutical composition comprising the compound or a pharmaceutically acceptable salt thereof of any one of embodiments 1-50, and a pharmaceutically acceptable carrier.

Embodiment 52. A method of inhibiting EGFR activity in a subject in need thereof, comprising administering an effective amount of the compound or a pharmaceutically acceptable salt thereof of any one of embodiments 1-50, or the pharmaceutical composition of embodiment 51 to the subject.

Embodiment 53. A method of treating an EGFR-related disorder in a subject in need thereof, comprising administering to the subject an effective amount of the compound or a pharmaceutically acceptable salt thereof of any one of embodiments 1-50, or the pharmaceutical composition of embodiment 51 to the subject.

Embodiment 54. The method according to embodiment 53, wherein the EGFR-related disorder is autoimmune diseases or cancers.

Embodiment 55. The method according to embodiment 54, wherein the cancer is selected from the group consisting of lung cancer, brain cancers, colorectal cancer, bladder cancer, urothelial cancer, breast cancer, prostate cancer, ovarian cancer, head and neck cancer, pancreatic cancer, gastric cancer and mesothelioma, including metastasis (in particular brain metastasis) and the like.

Embodiment 56. The method according to any one of embodiments 53-55, wherein the compound is administered simultaneously, separately or sequentially with one or more additional therapeutic agents.

Embodiment 57. The method according to embodiment 56, wherein the one or more additional therapeutic agents are selected from the group consisting of EGFR TKIs, EGFR antibodies, MEK inhibitors, c-MET inhibitors, mitotic kinase inhibitors, immunotherapeutic agents, anti-angiogenic agents, apoptosis inducers, mTOR inhibitors, histone deacetylase inhibitors, IL6 inhibitors, and JAK inhibitors.

Furthermore, embodiments of the various aspects provided by the present disclosure are also described in any of the following paragraphs.

Item 1. A compound of Formula (II)

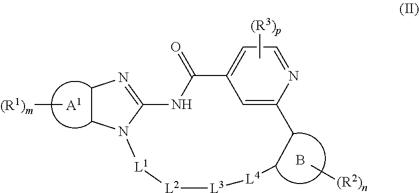

or a pharmaceutically acceptable salt thereof, wherein:
Ring A¹ is 7-12 membered cycloalkyl, 7-12 membered heterocyclyl, 7-12 membered aryl or 7-12 membered heteroaryl;
Ring B is selected from the group consisting of cycloalkyl, heterocyclyl, aryl, and heteroaryl;
L¹ is selected from the group consisting of a bond, alkyl, alkenyl, alkynyl, heteroalkyl, heteroalkenyl and heteroalkynyl, wherein the alkyl, alkenyl, alkynyl, heteroalkyl, heteroalkenyl and heteroalkynyl are optionally substituted with one or more groups independently selected from hydroxyl, halogen, cyano and amino;
L² is selected from the group consisting of a bond, N(R^{A}), alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more R^{B};
wherein R^{A} is selected from a group consisting of hydrogen, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, amino, alkyl and haloalkyl;
wherein each R^{B} is independently selected from a group consisting of hydroxyl, halogen, cyano, amino, alkyl, alkenyl, alkynyl, heteroalkyl, heteroalkenyl, cycloalkyl, heterocyclyl, aryl and heteroaryl;
L³ is selected from the group consisting of a bond, alkyl, alkenyl, alkynyl, heteroalkyl, heteroalkenyl and heteroalkynyl, wherein the alkyl, alkenyl, alkynyl, heteroalkyl, heteroalkenyl and heteroalkynyl are optionally substituted with one or more groups independently selected from hydroxyl, halogen, cyano and amino; L⁴ is selected from O, S, or N(R^{C});
wherein R^{C} is selected from a group consisting of hydrogen, alkyl, alkenyl, alkynyl, haloalkyl, heteroalkyl, heteroalkenyl, heteroalkynyl, cycloalkyl, heterocyclyl, aryl, and heteroaryl;
each R¹ is independently selected from the group consisting of hydroxy, halogen, cyano, amino, —N(R^{D})₂, alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more R^{E};
wherein each R^{D} is independently selected from a group consisting of hydrogen, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, —N(R^{F})₂ or —OR^{G};
wherein each R^{E} is independently selected from a group consisting of hydrogen, hydroxyl, halogen, cyano, amino, —N(R^{F})₂, -alkyl-N(R^{F})₂, —C(O)OR^{G}, alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, amino and alkyl;
wherein each of R^{F} and R^{G} is independently selected from a group consisting of alkyl, alkenyl, alkynyl, heteroalkyl, heteroalkenyl, heteroalkynyl, cycloalkyl, heterocyclyl, aryl and heteroaryl;
each R² is independently selected from the group consisting of hydrogen, hydroxy, halogen, cyano, amino, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, amino, alkyl and haloalkyl;
each R³ is selected from the group consisting of hydrogen, alkyl, alkenyl, alkynyl, heteroalkyl, heteroalkenyl, heteroalkynyl, cycloalkyl and heterocyclyl, wherein the alkyl, alkenyl, alkynyl, heteroalkyl, heteroalkenyl, heteroalkynyl, cycloalkyl and heterocyclyl are optionally substituted with one or more groups independently selected from hydroxyl, halogen, cyano, amino, alkyl and haloalkyl;
m is an integer from 0 to 5;
n is an integer from 0 to 4; and
p is an integer from 0 to 3.

Item 2. The compound of Item 1, or a pharmaceutically acceptable salt thereof, wherein Ring A¹ is 7-12 membered heteroaryl.

Item 3. The compound of Item 2, or a pharmaceutically acceptable salt thereof, wherein Ring A¹ is tetrahydroisoquinolinyl.

Item 4. The compound of Item 3, or a pharmaceutically acceptable salt thereof, wherein Ring A¹ is

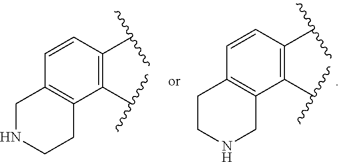 [SMILES: *c1ccc2c(c1*)CCNC2 ; *c1ccc2c(c1*)CNCC2]

Item 5. The compound of any one of preceding Items, or a pharmaceutically acceptable salt thereof, wherein m is 0.

Item 6. The compound of Item 1, or a pharmaceutically acceptable salt thereof, wherein m is 1.

Item 7. The compound of Item 6, or a pharmaceutically acceptable salt thereof, wherein R¹ is alkyl optionally substituted with one or more N(R^{F})₂.

Item 8. The compound of Item 7, or a pharmaceutically acceptable salt thereof, wherein R¹ is ethyl optionally substituted with one or more N(R^{F})₂.

Item 9. The compound of Item 7 or 8, or a pharmaceutically acceptable salt thereof, wherein R^{F} is alkyl.

Item 10. The compound of Item 9, or a pharmaceutically acceptable salt thereof, wherein R^{F} is methyl.

Item 11. The compound of Item 6, or a pharmaceutically acceptable salt thereof, wherein R¹ is heterocyclyl.

Item 12. The compound of Item 11, or a pharmaceutically acceptable salt thereof, wherein R¹ is oxetanyl.

Item 13. The compound of Item 12, or a pharmaceutically acceptable salt thereof, wherein R¹ is

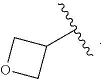 [SMILES: *C1COC1]

Item 14. The compound of Item 1, or a pharmaceutically acceptable salt thereof, wherein L¹ is alkyl.

Item 15. The compound of Item 14, or a pharmaceutically acceptable salt thereof, wherein L¹ is

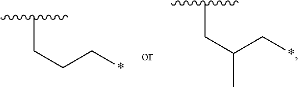

wherein * end of L¹ is connected to L².

Item 16. The compound of Item 1 or a pharmaceutically acceptable salt thereof, wherein L² is a bond.

Item 17. The compound of Item 1, or a pharmaceutically acceptable salt thereof, wherein L³ is alkyl.

Item 18. The compound of Item 11, or a pharmaceutically acceptable salt thereof, wherein L³ is ethyl.

Item 19. The compound of Item 1, or a pharmaceutically acceptable salt thereof, wherein L⁴ is O or NH.

Item 20. The compound of Item 1, or a pharmaceutically acceptable salt thereof, wherein Ring B is aryl or heteroaryl.

Item 21. The compound of Item 20, or a pharmaceutically acceptable salt thereof, wherein Ring B is phenyl, pyridinyl or pyrazolyl.

Item 22. The compound of Item 21, or a pharmaceutically acceptable salt thereof, wherein Ring B is selected from a group consisting of

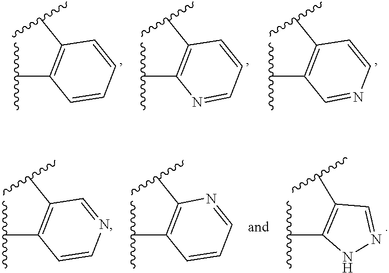

Item 23. The compound of Item 1, or a pharmaceutically acceptable salt thereof, wherein n is 1.

Item 24. The compound of Item 1, or a pharmaceutically acceptable salt thereof, wherein R² is alkyl.

Item 25. The compound of Item 24, or a pharmaceutically acceptable salt thereof, wherein R² is methyl.

Item 26. The compound of Item 1, or a pharmaceutically acceptable salt thereof, wherein R³ is alkyl.

Item 27. The compound of Item 26, or a pharmaceutically acceptable salt thereof, wherein R³ is methyl.

Item 28. The compound of Item 1, or a pharmaceutically acceptable salt thereof, wherein m is 0 or 1.

Item 29. The compound of Item 1, or a pharmaceutically acceptable salt thereof, wherein n is 1.

Item 30. The compound of Item 1, or a pharmaceutically acceptable salt thereof, wherein p is 1.

Item 31. A compound of Formula (III)

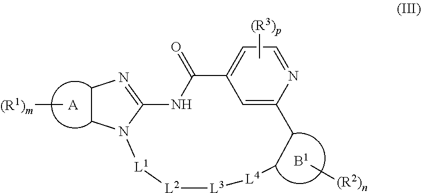

or a pharmaceutically acceptable salt thereof, wherein:
Ring A is selected from the group consisting of cycloalkyl, heterocyclyl, aryl and heteroaryl;
Ring B¹ is 7-12 membered cycloalkyl, 7-12 membered heterocyclyl, 7-12 membered aryl or 7-12 membered heteroaryl;
L¹ is selected from the group consisting of a bond, alkyl, alkenyl, alkynyl, heteroalkyl, heteroalkenyl and heteroalkynyl, wherein the alkyl, alkenyl, alkynyl, heteroalkyl, heteroalkenyl and heteroalkynyl are optionally substituted with one or more groups independently selected from hydroxyl, halogen, cyano and amino;
L² is selected from the group consisting of a bond, N(R^{A}), alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more R^{B};
wherein R^{A} is selected from a group consisting of hydrogen, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, amino, alkyl and haloalkyl;
wherein each R^{B} is independently selected from a group consisting of hydroxyl, halogen, cyano, amino, alkyl, alkenyl, alkynyl, heteroalkyl, heteroalkenyl, cycloalkyl, heterocyclyl, aryl and heteroaryl;
L³ is selected from the group consisting of a bond, alkyl, alkenyl, alkynyl, heteroalkyl, heteroalkenyl and heteroalkynyl, wherein the alkyl, alkenyl, alkynyl, heteroalkyl, heteroalkenyl and heteroalkynyl are optionally substituted with one or more groups independently selected from hydroxyl, halogen, cyano and amino;
L⁴ is selected from O, S, or N(R^{C});
wherein R^{C} is selected from a group consisting of hydrogen, alkyl, alkenyl, alkynyl, haloalkyl, heteroalkyl, heteroalkenyl, heteroalkynyl, cycloalkyl, heterocyclyl, aryl, and heteroaryl;
each R¹ is independently selected from the group consisting of hydroxy, halogen, cyano, amino, —N(R^{D})₂, alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more R^{E};
wherein each R^{D} is independently selected from a group consisting of hydrogen, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, —N(R^{F})₂ or —OR^{G};
wherein each R^{E} is independently selected from a group consisting of hydrogen, hydroxyl, halogen, cyano, amino, —N(R^{F})₂, -alkyl-N(R^{F})₂, —C(O)OR^{G}, alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, amino and alkyl;
wherein each of R^{F} and R^{G} is independently selected from a group consisting of alkyl, alkenyl, alkynyl, heteroalkyl, heteroalkenyl, heteroalkynyl, cycloalkyl, heterocyclyl, aryl and heteroaryl;
each R² is independently selected from the group consisting of hydrogen, hydroxy, halogen, cyano, amino, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, amino, alkyl and haloalkyl;
each R³ is selected from the group consisting of hydrogen, halogen, alkyl, alkenyl, alkynyl, heteroalkyl, heteroalkenyl, heteroalkynyl, cycloalkyl and heterocyclyl, wherein the alkyl, alkenyl, alkynyl, heteroalkyl, heteroalkenyl, heteroalkynyl, cycloalkyl and heterocyclyl are optionally substituted with one or more groups independently selected from hydroxyl, halogen, cyano, amino, alkyl and haloalkyl;
m is an integer from 0 to 5;
n is an integer from 0 to 4; and
p is an integer from 0 to 3.

Item 32. The compound of Item 31, or a pharmaceutically acceptable salt thereof, wherein Ring A is aryl or heteroaryl.

Item 33. The compound of Item 32, or a pharmaceutically acceptable salt thereof, wherein Ring A is selected from a group consisting of furanyl, thiophenyl, pyrrolyl, phenyl, pyridinyl, pyranyl, pyrimidinyl, pyridazinyl, pyrazinyl and tetrahydroisoquinoline.

Item 34. The compound of Item 31, or a pharmaceutically acceptable salt thereof, wherein m is 0.

Item 35. The compound of Item 31, or a pharmaceutically acceptable salt thereof, wherein R¹ is halogen and m is 1.

Item 36. The compound of Item 35, or a pharmaceutically acceptable salt thereof, wherein R¹ is bromo.

Item 37. The compound of Item 31, or a pharmaceutically acceptable salt thereof, wherein R¹ is —N(R^{D})₂ and m is 1.

Item 38. The compound of Item 37, or a pharmaceutically acceptable salt thereof, wherein each R^{D} is independently alkyl optionally substituted with one or more groups independently selected from —N(R^{F})₂ or —OR^{G}.

Item 39. The compound of Item 38, or a pharmaceutically acceptable salt thereof, wherein each R^{D} is independently methyl, methoxyethyl, or N,N-dimethylaminopropyl.

Item 40. The compound of Item 31, or a pharmaceutically acceptable salt thereof, wherein R¹ is heterocyclyl optionally substituted with one or more R^{E} and m is 1.

Item 41. The compound of Item 40, or a pharmaceutically acceptable salt thereof, wherein the heterocyclyl is selected from a group consisting of

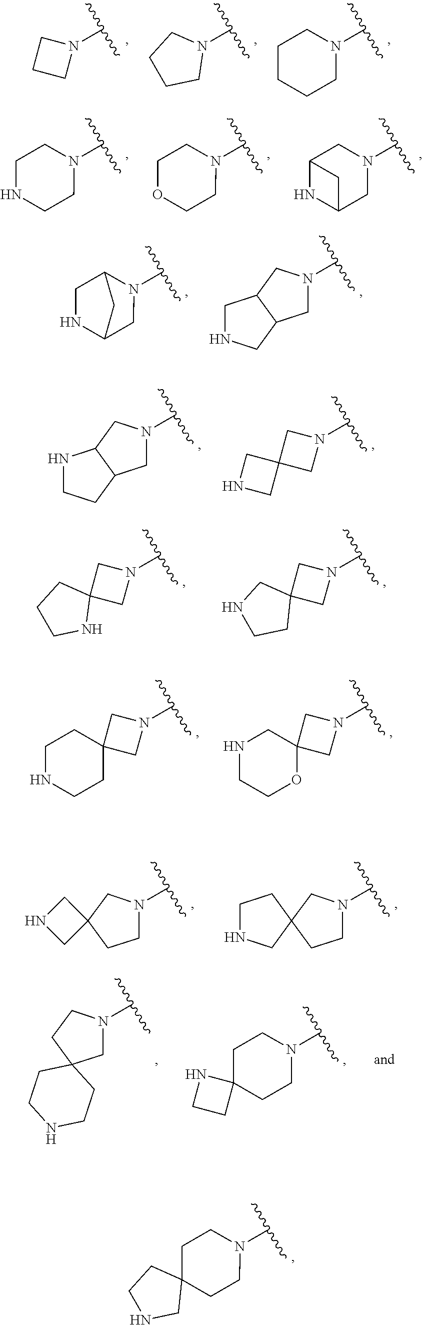

each of which is optionally substituted with one or more R^{E}.

Item 42. The compound of Item 40, or a pharmaceutically acceptable salt thereof, wherein R^{E} is halogen.

Item 43. The compound of Item 42, or a pharmaceutically acceptable salt thereof, wherein R^{E} is F.

Item 44. The compound of Item 40, or a pharmaceutically acceptable salt thereof, wherein R^{E} is —N(R^{F})₂, -alkyl-N(R^{F})₂, —C(O)OR^{G}, or alkyl optionally substituted with one or more halogen.

Item 45. The compound of Item 44, or a pharmaceutically acceptable salt thereof, wherein each of R^{F} and R^{G} is alkyl.

Item 46. The compound of Item 45, or a pharmaceutically acceptable salt thereof, wherein each of R^{F} and R^{G} is C₁₋₃ alkyl.

Item 47. The compound of Item 44, or a pharmaceutically acceptable salt thereof, wherein R^{E} is selected from a group consisting of —N(CH₃)₂, —C₁₋₃ alkyl-N(CH₃)₂, —C(O)O(tert-butyl), methyl, ethyl, or trifluoroethyl.

Item 48. The compound of Item 40, or a pharmaceutically acceptable salt thereof, wherein R^{E} is cycloalkyl or heterocyclyl, wherein the cycloalkyl and heterocyclyl are optionally substituted with one or more alkyl or halogen.

Item 49. The compound of Item 48, or a pharmaceutically acceptable salt thereof, wherein R^{E} is selected from cyclopropyl, morpholine, piperazine, oxetyl or azetidinyl, each of which is optionally substituted with one or more alkyl or halogen.

Item 50. The compound of Item 31, or a pharmaceutically acceptable salt thereof, wherein R¹ is selected from the group consisting of

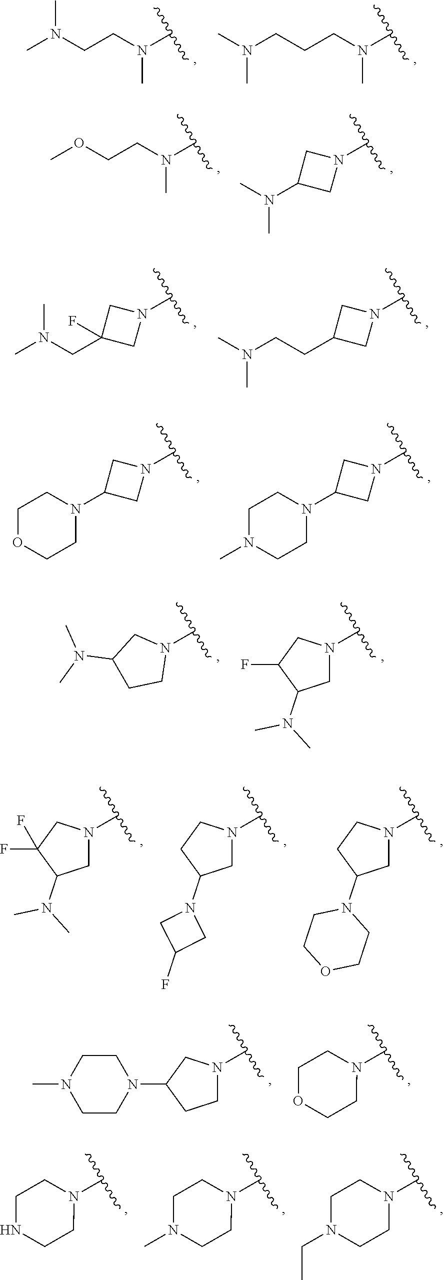

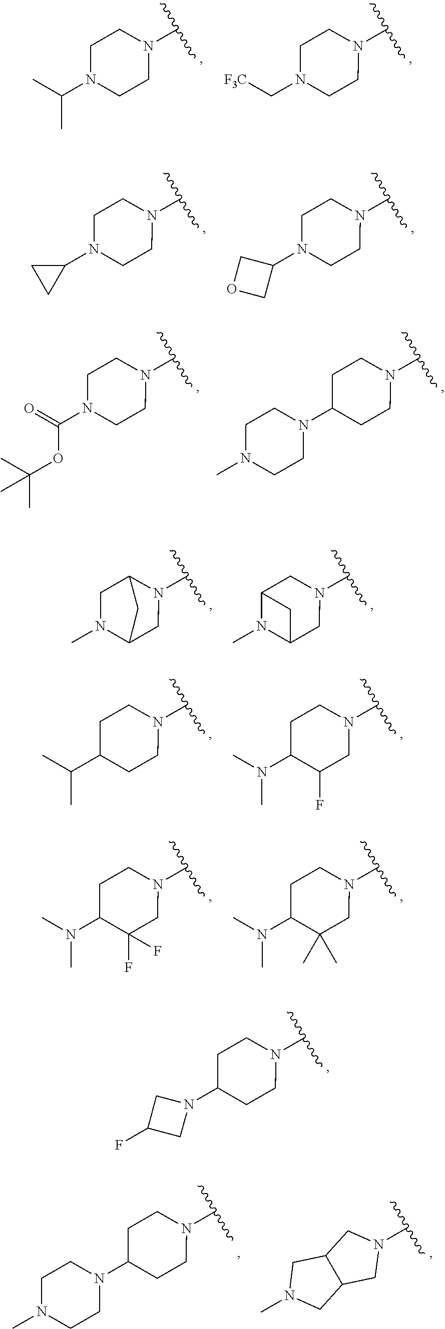

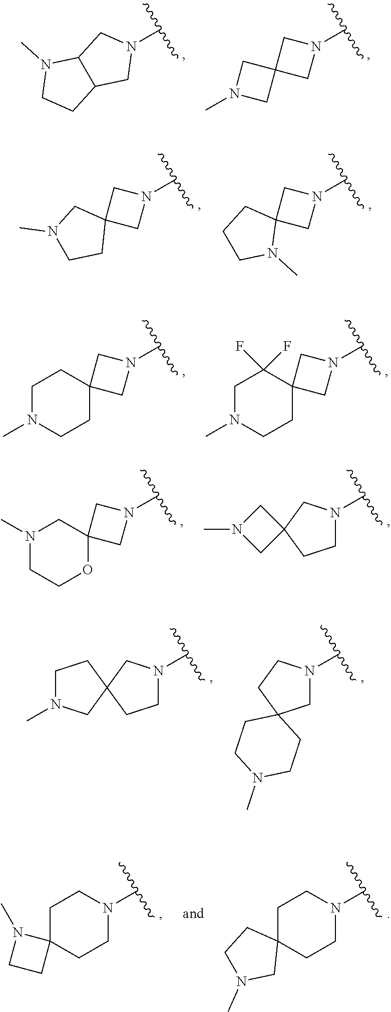

Item 51. The compound of Item 31, or a pharmaceutically acceptable salt thereof, wherein Ring B¹ is 7-12 membered heteroaryl.

Item 52. The compound of Item 51, or a pharmaceutically acceptable salt thereof, wherein Ring B¹ is

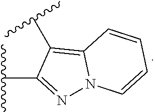 [SMILES: *c1nn2ccccc2c1*]

Item 53. The compound of Item 31, or a pharmaceutically acceptable salt thereof, wherein R² is halogen and n is 1 or 2.

Item 54. The compound of Item 53, or a pharmaceutically acceptable salt thereof, wherein R² is F.

Item 55. The compound of Item 31, or a pharmaceutically acceptable salt thereof, wherein R² is alkyl or cycloalkyl, wherein the alkyl and cycloalkyl are optionally substituted with one or more halogen.

Item 56. The compound of Item 55, or a pharmaceutically acceptable salt thereof, wherein R² is methyl, ethyl, cyclopropyl or trifluoroethyl.

Item 57. The compound of Item 31, or a pharmaceutically acceptable salt thereof, wherein L¹ is alkyl.

Item 58. The compound of Item 57, or a pharmaceutically acceptable salt thereof, wherein L¹ is propyl.

Item 59. The compound of Item 31, or a pharmaceutically acceptable salt thereof, wherein L² is a bond.

Item 60. The compound of Item 31, or a pharmaceutically acceptable salt thereof, wherein L³ is alkyl.

Item 61. The compound of Item 60, or a pharmaceutically acceptable salt thereof, wherein L³ is ethyl.

Item 62. The compound of Item 31, or a pharmaceutically acceptable salt thereof, wherein L⁴ is O or NH.

Item 63. The compound of Item 31, or a pharmaceutically acceptable salt thereof, wherein R³ is alkyl.

Item 64. The compound of Item 63, or a pharmaceutically acceptable salt thereof, wherein R³ is methyl.

Item 65. The compound of Item 31, or a pharmaceutically acceptable salt thereof, wherein R³ is halogen.

Item 66. The compound of Item 65, or a pharmaceutically acceptable salt thereof, wherein R³ is Cl.

Item 67. The compound of Item 31, or a pharmaceutically acceptable salt thereof, wherein m is 0.

Item 68. The compound of Item 31, or a pharmaceutically acceptable salt thereof, wherein n is 0.

Item 69. The compound of Item 31, or a pharmaceutically acceptable salt thereof, wherein p is 1.

Item 70. A compound of Formula (IV):

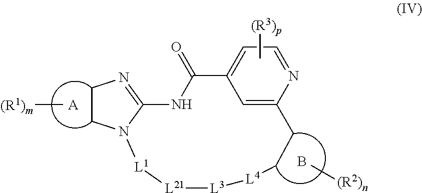

or a pharmaceutically acceptable salt thereof, wherein:
Ring A is selected from the group consisting of cycloalkyl, heterocyclyl, aryl and heteroaryl;
Ring B is selected from the group consisting of cycloalkyl, heterocyclyl, aryl, and heteroaryl;
L¹ is selected from the group consisting of a bond, alkyl, alkenyl, alkynyl, heteroalkyl, heteroalkenyl and heteroalkynyl, wherein the alkyl, alkenyl, alkynyl, heteroalkyl, heteroalkenyl and heteroalkynyl are optionally substituted with one or more groups independently selected from hydroxyl, halogen, cyano and amino;
L²¹ is 7-12 membered cycloalkyl or 7-12 membered heterocyclyl, wherein the cycloalkyl and heterocyclyl are optionally substituted with one or more R^{B};
wherein each R^{B} is independently selected from a group consisting of hydroxyl, halogen, cyano, amino, alkyl, alkenyl, alkynyl, heteroalkyl, heteroalkenyl, cycloalkyl, heterocyclyl, aryl and heteroaryl;
L³ is selected from the group consisting of a bond, alkyl, alkenyl, alkynyl, heteroalkyl, heteroalkenyl and heteroalkynyl, wherein the alkyl, alkenyl, alkynyl, heteroalkyl, heteroalkenyl and heteroalkynyl are optionally substituted with one or more groups independently selected from hydroxyl, halogen, cyano and amino;
L⁴ is selected from O, S, or N(R^{C});
wherein R^{C} is selected from a group consisting of hydrogen, alkyl, alkenyl, alkynyl, haloalkyl, heteroalkyl, heteroalkenyl, heteroalkynyl, cycloalkyl, heterocyclyl, aryl, and heteroaryl;
each R¹ is independently selected from the group consisting of hydroxy, halogen, cyano, amino, —N(R^{D})₂, alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more R^{E};
wherein each R^{D} is independently selected from a group consisting of hydrogen, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, —N(R^{F})₂ or —OR^{G};
wherein each R^{E} is independently selected from a group consisting of hydrogen, hydroxyl, halogen, cyano, amino, —N(R^{F})₂, -alkyl-N(R^{F})₂, —C(O)OR^{G}, alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, amino and alkyl;
wherein each of R^{F} and R^{G} is independently selected from a group consisting of alkyl, alkenyl, alkynyl, heteroalkyl, heteroalkenyl, heteroalkynyl, cycloalkyl, heterocyclyl, aryl and heteroaryl;
each R² is independently selected from the group consisting of hydrogen, hydroxy, halogen, cyano, amino, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, amino, alkyl and haloalkyl;
R³ is selected from the group consisting of hydrogen, alkyl, alkenyl, alkynyl, heteroalkyl, heteroalkenyl, heteroalkynyl, cycloalkyl and heterocyclyl, wherein the alkyl, alkenyl, alkynyl, heteroalkyl, heteroalkenyl, heteroalkynyl, cycloalkyl and heterocyclyl are optionally substituted with one or more groups independently selected from hydroxyl, halogen, cyano, amino, alkyl and haloalkyl;
m is an integer from 0 to 5;
n is an integer from 0 to 4; and
p is an integer from 0 to 3.

Item 71. The compound of Item 70, or a pharmaceutically acceptable salt thereof, wherein Ring A is aryl.

Item 72. The compound of Item 71, or a pharmaceutically acceptable salt thereof, wherein Ring A is phenyl.

Item 73. The compound of Item 70, or a pharmaceutically acceptable salt thereof, wherein R¹ is halogen and m is 1.

Item 74. The compound of Item 73, or a pharmaceutically acceptable salt thereof, wherein R¹ is bromo.

Item 75. The compound of Item 70, or a pharmaceutically acceptable salt thereof, wherein R¹ is —N(R^{D})₂ and m is 1.

Item 76. The compound of Item 75, or a pharmaceutically acceptable salt thereof, wherein each R^{D} is independently alkyl optionally substituted with one or more groups independently selected from —N(R^{F})₂ or —OR^{G}.

Item 77. The compound of Item 76, or a pharmaceutically acceptable salt thereof, wherein each R^{D} is independently methyl, methoxyethyl, or N,N-dimethylaminopropyl.

Item 78. The compound of Item 70, or a pharmaceutically acceptable salt thereof, wherein R¹ is heterocyclyl optionally substituted with one or more R^{E} and m is 1.

Item 79. The compound of Item 78, or a pharmaceutically acceptable salt thereof, wherein the heterocyclyl is selected from a group consisting of

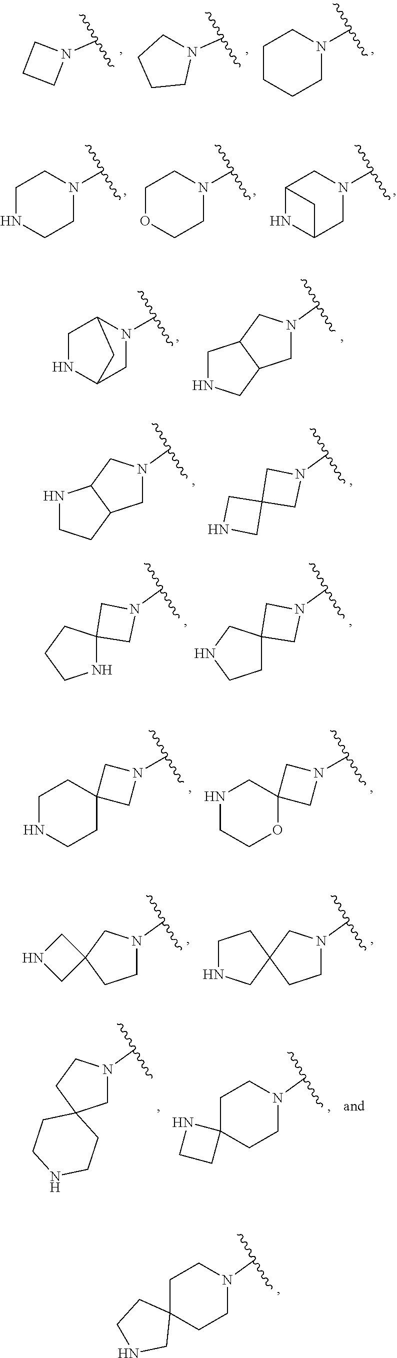

each of which is optionally substituted with one or more R^{E}.

Item 80. The compound of Item 78, or a pharmaceutically acceptable salt thereof, wherein R^{E} is halogen.

Item 81. The compound of Item 80, or a pharmaceutically acceptable salt thereof, wherein R^{E} is F.

Item 82. The compound of Item 78, or a pharmaceutically acceptable salt thereof, wherein R^{E} is —N(R^{F})₂, -alkyl-N(R^{F})₂, —C(O)OR^{G}, or alkyl optionally substituted with one or more halogen.

Item 83. The compound of Item 82, or a pharmaceutically acceptable salt thereof, wherein each of R^{F} and R^{G} is alkyl.

Item 84. The compound of Item 83, or a pharmaceutically acceptable salt thereof, wherein each of R^{F} and R^{G} is C₁₋₃ alkyl.

Item 85. The compound of Item 82, or a pharmaceutically acceptable salt thereof, wherein R^{E} is selected from a group consisting of —N(CH₃)₂, —C₁₋₃ alkyl-N(CH₃)₂, —C(O)O(tert-butyl), methyl, ethyl, or trifluoroethyl.

Item 86. The compound of Item 78, or a pharmaceutically acceptable salt thereof, wherein R^{E} is cycloalkyl or heterocyclyl, wherein the cycloalkyl and heterocyclyl are optionally substituted with one or more alkyl or halogen.

Item 87. The compound of Item 86, or a pharmaceutically acceptable salt thereof, wherein R^{E} is selected from cyclopropyl, morpholine, piperazine, oxetyl or azetidinyl, each of which is optionally substituted with one or more alkyl or halogen.

Item 88 The compound of Item 70, or a pharmaceutically acceptable salt thereof, wherein R¹ is selected from the group consisting of

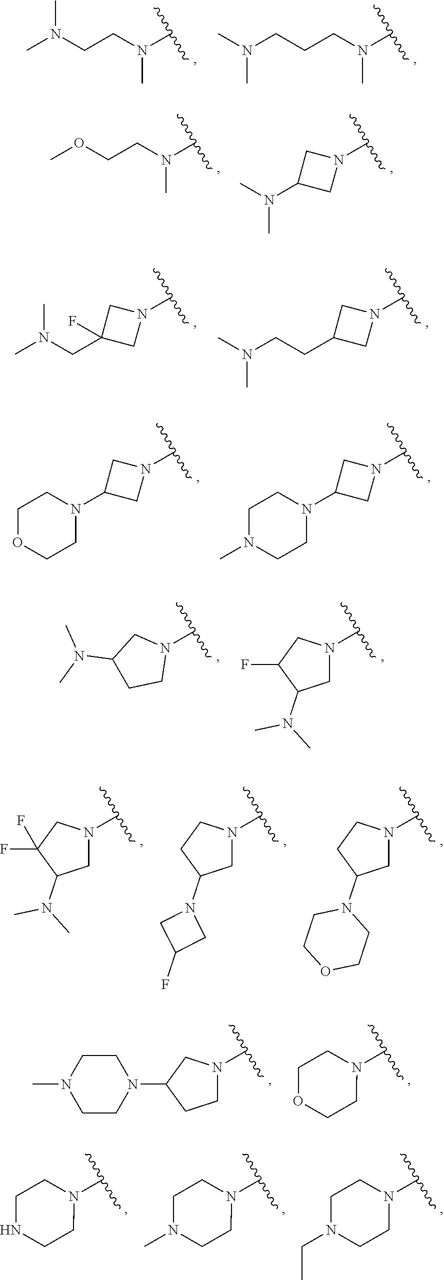

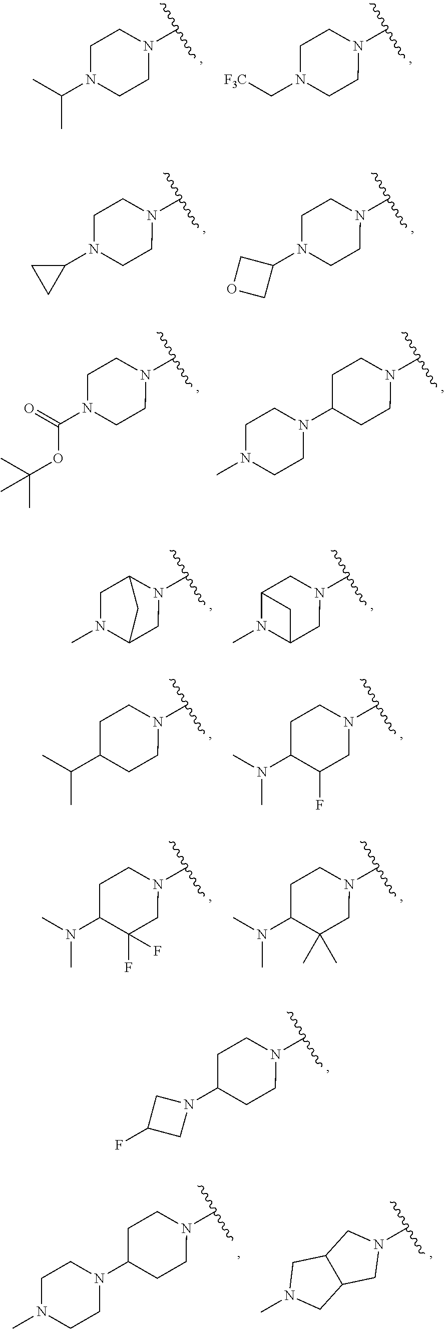

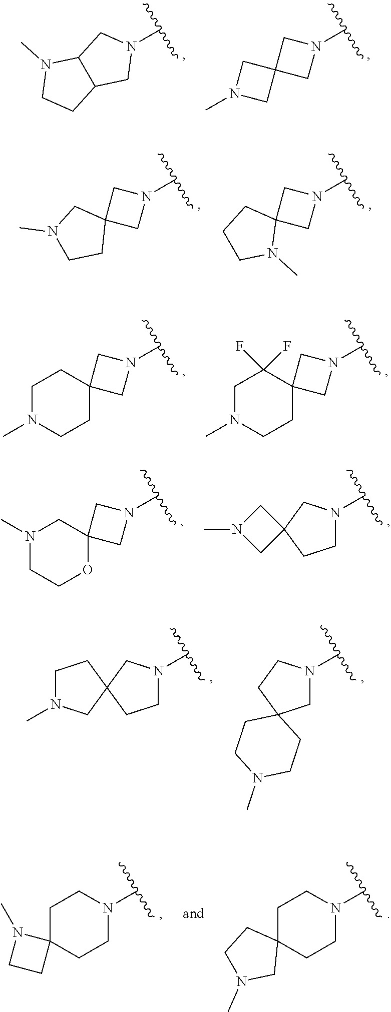

Item 89. The compound of Item 70, or a pharmaceutically acceptable salt thereof, wherein Ring B is aryl or heteroaryl.

Item 90. The compound of Item 89, or a pharmaceutically acceptable salt thereof, wherein Ring B is phenyl, pyridinyl or pyrazolyl.

Item 91. The compound of Item 90, or a pharmaceutically acceptable salt thereof, wherein Ring B is selected from a group consisting of

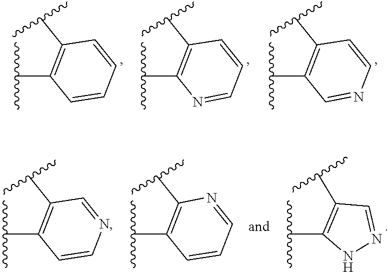

Item 92. The compound of Item 70, or a pharmaceutically acceptable salt thereof, wherein L¹ is a bond.

Item 93. The compound of Item 70, or a pharmaceutically acceptable salt thereof, wherein L²¹ is 7-10 membered heterocyclyl containing one or more heteroatoms selected from N or O.

Item 94. The compound of Item 93, or a pharmaceutically acceptable salt thereof, wherein L²¹ is selected from a group consisting of

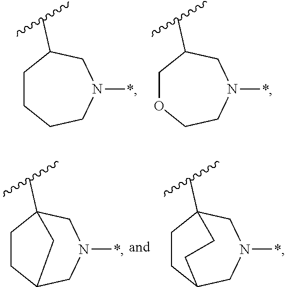

wherein * end of L² is connected to L³.

Item 95. The compound of Item 70, or a pharmaceutically acceptable salt thereof, wherein L³ is alkyl.

Item 96. The compound of Item 95, or a pharmaceutically acceptable salt thereof, wherein L³ is ethyl.

Item 97. The compound of Item 70, or a pharmaceutically acceptable salt thereof, wherein L⁴ is O or NH.

Item 98. The compound of Item 70, or a pharmaceutically acceptable salt thereof, wherein R¹ is heterocyclyl optionally substituted with one or more R^{E} and m is 1.

Item 99. The compound of Item 70, or a pharmaceutically acceptable salt thereof, wherein R³ is alkyl.

Item 100. The compound of Item 99, or a pharmaceutically acceptable salt thereof, wherein R³ is methyl.

Item 101. The compound of Item 70, or a pharmaceutically acceptable salt thereof, wherein m is 0 or 1.

Item 102. The compound of Item 70, or a pharmaceutically acceptable salt thereof, wherein n is 1.

Item 103. The compound of Item 70, or a pharmaceutically acceptable salt thereof, wherein p is 1.

Item 104. A compound or a pharmaceutically acceptable salt thereof, wherein the compound is selected from the group consisting of:

[chemical structure]

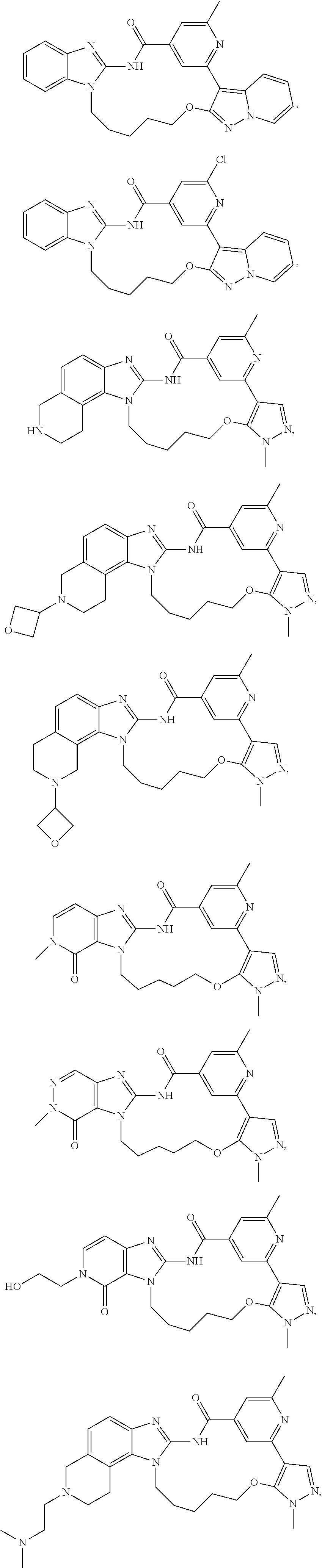

Item 105. A pharmaceutical composition comprising the compound or a pharmaceutically acceptable salt thereof of any one of Items 1-104, and a pharmaceutically acceptable carrier.

Item 106. A method of inhibiting EGFR activity in a subject in need thereof, comprising administering an effective amount of the compound or a pharmaceutically acceptable salt thereof of any one of Items 1-104, or the pharmaceutical composition of Item 105 to the subject.

Item 107. A method of treating an EGFR-related disorder in a subject in need thereof, comprising administering to the subject an effective amount of the compound or a pharmaceutically acceptable salt thereof of any one of Items 1-104, or the pharmaceutical composition of Item 105 to the subject.

Item 108. The method according to Item 107, wherein the EGFR-related disorder is autoimmune diseases or cancers.

Item 109. The method according to Item 108, wherein the cancer is selected from the group consisting of lung cancer, brain cancers, colorectal cancer, bladder cancer, urothelial cancer, breast cancer, prostate cancer, ovarian cancer, head and neck cancer, pancreatic cancer, gastric cancer and mesothelioma, including metastasis (in particular brain metastasis) and the like.

Item 110. The method according to any one of Items 107-109, wherein the compound is administered simultaneously, separately or sequentially with one or more additional therapeutic agents.

Item 111. The method according to Item 110, wherein the one or more additional therapeutic agents are selected from the group consisting of EGFR TKIs, EGFR antibodies, MEK inhibitors, c-MET inhibitors, mitotic kinase inhibitors, immunotherapeutic agents, anti-angiogenic agents, apoptosis inducers, mTOR inhibitors, histone deacetylase inhibitors, IL6 inhibitors, JAK inhibitors.

### EXAMPLES

The followings further explain the general methods of the present disclosure. The compounds of the present disclosure may be prepared by the methods known in the art. The following illustrates the detailed preparation methods of the preferred compounds of the present disclosure. However, they are by no means limiting the preparation methods of the compounds of the present disclosure.

### Synthetic Examples

For the purpose of illustration, the following examples are included. The Examples provided herein describe the synthesis of compounds disclosed herein as well as intermediates used to prepare the compounds. However, it is to be understood that these examples do not limit the present disclosure and are only meant to suggest a method of practicing the present disclosure. Persons skilled in the art will recognize that the chemical reactions described may be readily adapted to prepare a number of other compounds of the present disclosure, and alternative methods for preparing the compounds of the present disclosure are deemed to be within the scope of the present disclosure. For example, the synthesis of non-exemplified compounds according to the present disclosure may be successfully performed by modifications apparent to those skilled in the art, e.g., by appropriately protecting interfering groups, by utilizing other suitable reagents and building blocks known in the art other than those described, and/or by making routine modifications of reaction conditions. Besides, persons skilled in the art will also understand that individual steps described herein or in the separate batches of a compound may be combined. Alternatively, other reactions disclosed herein or known in the art will be recognized as having applicability for preparing other compounds of the present disclosure. The following description is, therefore, not intended to limit the scope of the present disclosure, but rather is specified by the claims appended hereto.

### Chemistry, Materials, and General Methods

Unless otherwise noted, all reagents and solvents were obtained from commercial suppliers such as PharmaBlock, Bide Pharmatech, Shanghai Send Pharm, Aldrich, Sigma, etc., and used without further purification. Dry organic solvents (THF, DMF, 1,4-dioxane etc.) were purchased from Anhui Senrise Technology, Beijing innochem science & Technology and J & K Scientific packaged under nitrogen in Sure/Seal bottles. All reactions involving air or moisture sensitive reagents were performed under a nitrogen atmosphere. ¹H NMR spectra were recorded on a Bruker AV III HD 400 MHz, Bruker AV NEO 400 MHz spectrometer at ambient temperature. Chemical shifts are reported in parts per million (ppm, 6 units). Data are reported as follows: chemical shift, number of protons, and multiplicity (s=singlet, d=doublet, dd=doublet of doublet, dt=doublet of triplet, t=triplet, q=quartet, br=broad, m=multiplet). Reactions were monitored using LCMS (Shimadzu 20AD) with UV detection at 254 nm and a low resonance electrospray mode (ESI). Most final compounds were purified to >95% purity, as determined by LCMS (3 min). LCMS (3 min) methods used the following: Shimadzu 20AD spectrometer, Shim—pack Scepter C18-120 3.3*33 mm, 3.0 um at 30 °C with a 1.5 mL/min flow rate; solvent A of water+6.5 mM NH₄HCN₃+Ammonia Hydroxide (pH=10), solvent B ofacetonitrile; 0.0-1.7 mi, 30% B-70% B; 1.7-2.3 min, 70% B-95% B; 2.3-2.8 min, 95% B; 2.8-3.0 min, 10% B. Flow from UV detector was split (1:3) to the MS detector, which was configured with ESI as ionizable source. CHIRAL methods used the following: Column: CHIRALPAK IC-3, 0.46 * 5 cm, 3 μm; at 25 °C with a 1.0 mL/min flow rate; tert-butyl methyl ether (0.2a diethylamine)/(ethanol/dichloromethane=1/1)=20/80. SFC methods used the following: Column: (R, R)-WHELK-01-Kromasil, 5*25 cm, 5 μm; Mobile Phase A: CO₂, Mobile Phase B: methanol/acetonitrile=1/1.

Abbreviations used in the synthesis of the compounds provided herein are listed below:

| Abbreviation | Full name |
|---|---|
| ACN | Acetonitrile |
| AcOH | Acetic acid |
| BINAP | (2,2′-bis(diphenylphosphino)-1,1′-binaphthyl) |
| BrettPhos Pd | [(2-Di-cyclohexylphosphino-3,6-dimethoxy-2′,4′,6′-triisopropyl- |
| G₃ | 1,1′-biphenyl)-2-(2′-amino-1,1′-biphenyl)]palladium(II) |
| | methanesulfonate methanesulfonate |
| BrCN | Cyanogen bromide |
| n-Bu₃P | Tri-n-butylphosphine |
| CH₃CN | Acetonitrile |
| Cs₂CO₃ | Caesium carbonate |
| DCM | Dichloromethane |
| DEA | Diethanolamine |
| DIAD | Diisopropyl azodicarboxylate |
| DIEA | N,N-Diisopropylethylamine |
| DIPEA | N,N-Diisopropylethylamine |
| DMA | N,N-Dimethylacetamide |
| DMF | N,N-Dimethylformamide |
| DMAP | N,N-dimethylpyridin-4-amine |
| DMSO | Dimethyl sulfoxide |
| DPPA | Diphenyl azidophosphate |
| EA | Ethyl acetate |
| equiv | equivalent |
| EtOAc | Ethyl acetate |
| EtOH | Ethanol |
| h, hr (hrs) | Hour (s) |
| HATU | 1-[Bis(dimethylamino)methylene]-1H-1,2,3-triazolo[4,5- |
| | b]pyridinium 3-oxid hexafluorophosphate |
| HCl | Hydrogen chloride, hydrochloric acid |
| Hex | Hexanes |
| HPLC | High performance liquid chromatography |
| IPA | Isopropyl alcohol |
| K₂CO₃ | Potassium carbonate |
| KOAc | Potassium acetate |
| LiOH | Lithium hydroxide |
| LiHMDS | Lithium hexamethyldisilazide |
| MeCN | Acetonitrile |
| MeOH | Methanol |
| 2-MeTHF | 2-Methyltetrahydrofuran |
| MeI | Methyl iodide |
| Mg(OTf)₂ | Magnesium trifluoromethanesulfonate |
| MTBE | Methyl tert-butyl ether |
| Na₂CO₃ | Sodium Carbonate |
| NaCl | Sodium chloride |
| NaI | Sodiumiodide |
| NaH | sodium hydride |
| NaHCO₃ | Sodium bicarbonate |
| NaOH | Sodium hydroxide |
| NBS | N-Bromosuccinimide |
| NIS | N-iodosuccinimide |
| NH₂NH₂•H₂O | Hydrazine hydrate |
| Na₂SO₄ | Sodium sulphate |
| NH₄HCO₃ | Ammonium bicarbonate |
| NH₃•H₂O | Ammonium hydroxide |
| NH4Cl | Ammonium chloride |
| NaBH4 | Sodium borohydride |
| Pd(dppf)Cl₂ | [1,1′-Bis(diphenylphosphino)ferrocene]dichloropalladium(II) |
| Pd(OAc)₂ | Palladium(II) acetate |
| PE | Petroleum ether |
| Prep-HPLC, | Preparative high-performance liquid chromatography |
| prep-HPLC | |
| PPh3 | triphenylphosphine |
| Prep-TLC, pre- | Preparative thin layer chromatography |
| TLC | |
| POM | polyformaldehyde |
| Rt, Rₜ, t_{R} | Retention time |
| r.t. | Room temperature |
| SEM-Cl | 2-(trimethylsilyl)ethoxymethyl chloride |
| TEA | Triethylamine |
| TFA | Trifluoroacetic acid |
| THF | Tetrahydrofuran |
| TMAD | N,N,N′,N′- tetramethylazodicarboxamide |
| TBAF | Tetrabutylammonium fluoride |
| TsCl | p-toluenesulfonyl chloride |
| TLC | Thin Layer Chromatography |
| Xantphos | 4,5-Bis(diphenylphosphino)-9,9-dimethylxanthene |

### Synthesis of Intermediates

### Intermediate M1

### Methyl 2-(5-hydroxy-1-methyl-1H-pyrazol-4-yl)-6-methylisonicotinate

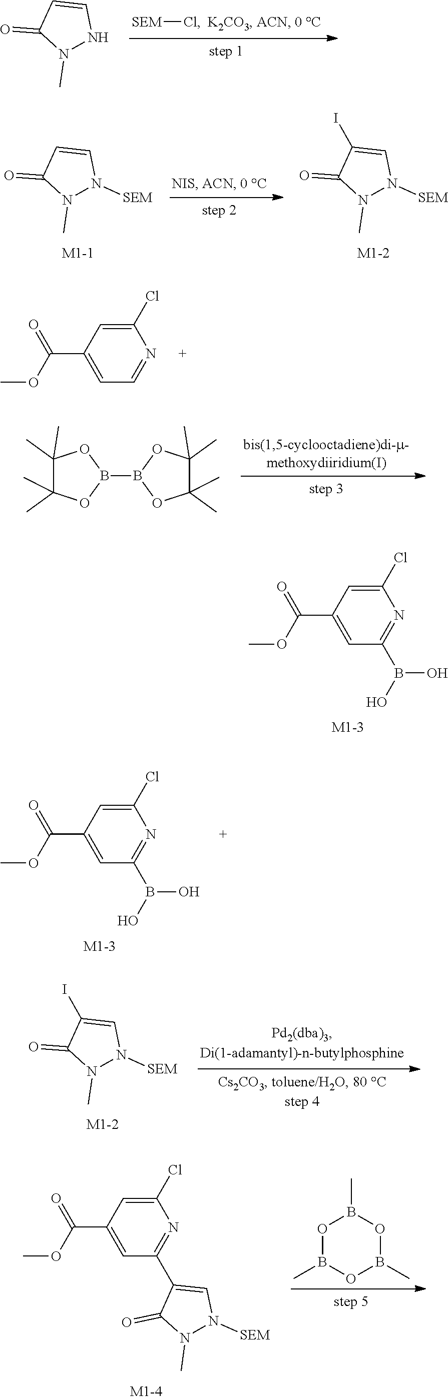

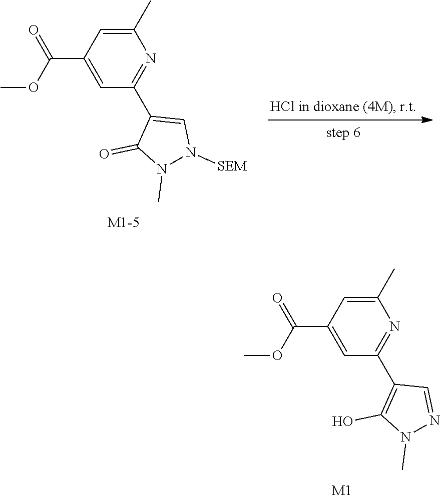 [SMILES: COC(=O)c1cc(C)nc(-c2cn(COCC[Si](C)(C)C)n(C)c2=O)c1 ; COC(=O)c1cc(C)nc(-c2cnn(C)c2O)c1 ; "M1"]

Step 1. Preparation of 2-methyl-1-{[2-(trimethylsilyl)ethoxy]methyl}pyrazol-3-one (M1-1). To a stirred solution of 2-methyl-1H-pyrazol-3-one (100 g, 1019 mmol, 1 equiv) and K₂CO₃ (352 g, 2548 mmol, 2.5 equiv) in acetonitrile (1000 mL) was added 2-(trimethylsily)ethoxymethyl chloride (254 g, 1528 mmol, 1.5 equiv) dropwise at 0 °C under nitrogen atmosphere. The resulting mixture was stirred for 3 h at room temperature. The reaction was quenched with water (200 mL) at 0 °C and the mixture was extracted with ethyl acetate (3×500 mL). The combined organic layers were washed with brine (3×500 mL) and dried over anhydrous Na₂SO₄. After filtration, the filtrate was concentrated under reduced pressure. The residue was purified by trituration with petroleum ether (200 mL) to give 130 g of 2-methyl-1-{[2-(trimethylsilyl)ethoxy]methyl}pyrazol-3-one (M1-1) as a yellow solid (55%). LCMS: m/z (ESI), [2M+H]⁺=457.25. ¹H NMR (DMSO-d₆, 400 MHz) δ 0.00 (9H, s), 0.80-0.85 (2H, m), 3.28 (3H, s), 3.40-3.50 (2H, m), 5.15 (2H, s), 5.29 (1H, d), 7.89 (1H, d).

Step 2. Preparation of 4-iodo-2-methyl-1-{[2-(trimethylsilyl)ethoxy]methyl}pyrazol-3-one (M1-2). To a stirred mixture of 2-methyl-1-{[2-(trimethylsilyl)ethoxy]methyl}pyrazol-3-one (47 g, 205.80 mmol, 1 equiv) in acetonitrile (400 mL) was added N-iodosuccinimide (50.93 g, 226.39 mmol, 1.1 equiv) at 0 °C under nitrogen atmosphere. The resulting mixture was stirred for 1 h at 0 °C under nitrogen atmosphere. The reaction was quenched with sodium thiosulfate aqueous solution (400 mL) at 0 °C and the mixture was extracted with dichloromethane (3×300 mL). The combined organic layers were washed with brine (400 mL) and dried over anhydrous Na₂SO₄. After filtration, the filtrate was concentrated under reduced pressure. The residue was purified by trituration with petroleum ether (300 mL) to give 72 g of 4-iodo-2-methyl-1-{[2-(trimethylsilyl)ethoxy]methyl}pyrazol-3-one (M1-2) as a yellow solid (97%). LCMS: m/z (ESI), [M+H]⁺=354.95. ¹H NMR (DMSO-d₆, 400 MHz) δ −0.04 (9H, s), 0.75-0.86 (2H, m), 3.32 (3H, s), 3.39-3.48 (2H, m), 5.13 (2H, s), 8.13 (1H, s).

Step 3. Preparation of (6-chloro-4-(methoxycarbonyl)pyridin-2-yl)boronic acid (M1-3). A mixture of bis(pinacolato)diboron (11.5 g, 453.7 mmol, 1.3 equiv) in tert-butyl methyl ether (100 mL) was warmed to 80 °C and stirred for 0.5 h, then colled to room temperature and added bis(1,5-cyclooctadiene)di-μ-methoxydiiridium(I) (1.16 g, 17 mmol, 0.05 equiv) and 4-tert-butyl-2-(4-tert-butylpyridin-2-yl)pyridine (1.4 g, 52 mmol, 0.15 equiv). The mixture was stirred for 0.5 h at 25 °C and then added methyl 2-chloropyridine-4-carboxylate (60 g, 349 mmol, 1.00 equiv) and stirred for 16 h at 80 °C. The mixture was cooled to room temperature and concentrated under reduced pressure to give 104 g (crude) of methyl 2-chloro-6-(4,4,5,5-tetramethyl-1,3,2-dioxaborolan-2-yl)pyridine-4-carboxylate (M1-3) as a brown oil. This crude material was used for the next step without purification. LCMS: m/z (ESI), [M+H]⁺=216.05.

Step 4. Preparation of methyl 2-chloro-6-(2-methyl-3-oxo-1-((2-(trimethylsilyl)ethoxy)methyl)-2,3-dihydro-1H-pyrazol-4-yl)isonicotinate (M1-4). A mixture of freshly prepared methyl 2-chloro-6-(4,4,5,5-tetramethyl-1,3,2-dioxaborolan-2-yl)pyridine-4-carboxylate (104 g crude, ~349 mmol, ~1 equiv), 4-iodo-2-methyl-1-{[2-(trimethylsilyl)ethoxy]methyl}pyrazol-3-one (M1-2,124.30 g, 350.877 mmol, 1 equiv), bis(adamantan-1-yl)(butyl)phosphane (12.58 g, 35.088 mmol, 0.1 equiv), Cs₂CO₃ (228.65 g, 701.754 mmol, 2 equiv), and Pd₂(dba)₃ (9.6 g, 10.4 mmol, 0.03 equiv) in toluene (600 mL) and water (150 mL) was stirred for 2 h at 80 °C under nitrogen atmosphere. The mixture was cooled to room temperature. The reaction was quenched with water (800 mL) and the mixture was extracted with ethyl acetate (3×600 mL). The combined organic layers were washed with brine (3×500 mL), dried over anhydrous Na₂SO₄, and concentrated under reduced pressure. The residue was purified on a silica gel column eluted with petroleum ether/ethyl acetate (1/4) to give 38 g of methyl 2-chloro-6-(2-methyl-3-oxo-1-((2-(trimethylsilyl)ethoxy)methyl)-2,3-dihydro-1H-pyrazol-4-yl)isonicotinate (M1-4) as a yellow solid (25%). LCMS: m/z (ESI), [M+H]⁺=398.05.

Step 5. Preparation of methyl 2-methyl-6-(2-methyl-3-oxo-1-((2-(trimethylsilyl)ethoxy)methyl)-2,3-dihydro-1H-pyrazol-4-yl)isonicotinate (M1-5). To a stirred mixture of methyl 2-chloro-6-(2-methyl-3-oxo-1-{[2-(trimethylsilyl)ethoxy]methyl}pyrazol-4-yl)pyridine-4-carboxylate (38 g, 95 mmol, 1.00 equiv), Pd(dppf)Cl₂ (3677 mg, 5.02 mmol, 0.05 equiv), PCy₃·HBF₄ (10.5 g, 28.64 mmol, 0.3 equiv), and K₂CO₃ (26.4 g, 190 mmol, 2 equiv) in 1,4-dioxane (500 mL) was added trimethyl-1,3,5,2,4,6-trioxatriborinane (23.9 g, 190 mmol, 2 equiv) at room temperature under nitrogen atmosphere. The mixture was stirred for 2 h at 100 °C and then cooled to room temperature. The reaction was quenched with water (300 mL) and the mixture was extracted with ethyl acetate (3×200 mL). The combined organic layers were washed with brine (3×300 mL), dried over anhydrous Na₂SO₄, and concentrated under reduced pressure.

The residue was purified on a silica gel column eluted with petroleum ether/ethyl acetate (1/3) to give 34 g of methyl 2-methyl-6-(2-methyl-3-oxo-1-((2-(trimethylsilyl)ethoxy)methyl)-2,3-dihydro-1H-pyrazol-4-yl)isonicotinate (M1-5) as a yellow solid (92%). LCMS: m/z (ESI), [M+H]⁺=378.15. ¹H NMR (DMSO-d₆, 400 MHz) δ 0.05 (9H, s), 0.80-0.90 (2H, m), 2.53 (3H, s), 3.41 (3H, s), 3.50 (2H, t), 3.89 (3H, s), 5.37 (2H, s), 7.44 (1H, d), 8.52-8.60 (2H, m).

Step 6. Preparation of methyl 2-(5-hydroxy-1-methyl-1H-pyrazol-4-yl)-6-methylisonicotinate (M1). A solution of methyl 2-methyl-6-(2-methyl-3-oxo-1-{[2-(trimethylsilyl)ethoxy]methyl}pyrazol-4-yl)pyridine-4-carboxylate (34 g, 90 mmol, 1.00 equiv) and HCl in 1,4-dioxane (340 mL) was stirred for 2 h at room temperature under nitrogen atmosphere. The resulting mixture was concentrated under reduced pressure. The residue was treated with saturated NaHCO₃ (aq.) solution (300 mL) and extracted with ethyl acetate (3×200 mL). The combined organic layers were washed with brine (3×500 mL), dried over anhydrous Na₂SO₄, and concentrated under reduced pressure to give 20 g methyl 2-(5-hydroxy-1-methyl-1H-pyrazol-4-yl)-6-methylisonicotinate (M1) as a red solid (80%). LCMS: m/z (EST), [M+H]⁺=248.05. ¹H NMR (DMSO-d₆, 400 MHz) δ 2.56 (3H, s), 3.48 (3H, s), 3.91 (3H, s), 7.31 (1H, s), 7.90-7.80 (2H, m).

### Intermediate M2

### Methyl 5-(5-hydroxy-1-methylpyrazol-4-yl)-1-methyl-6-oxopyridine-3-carboxylate

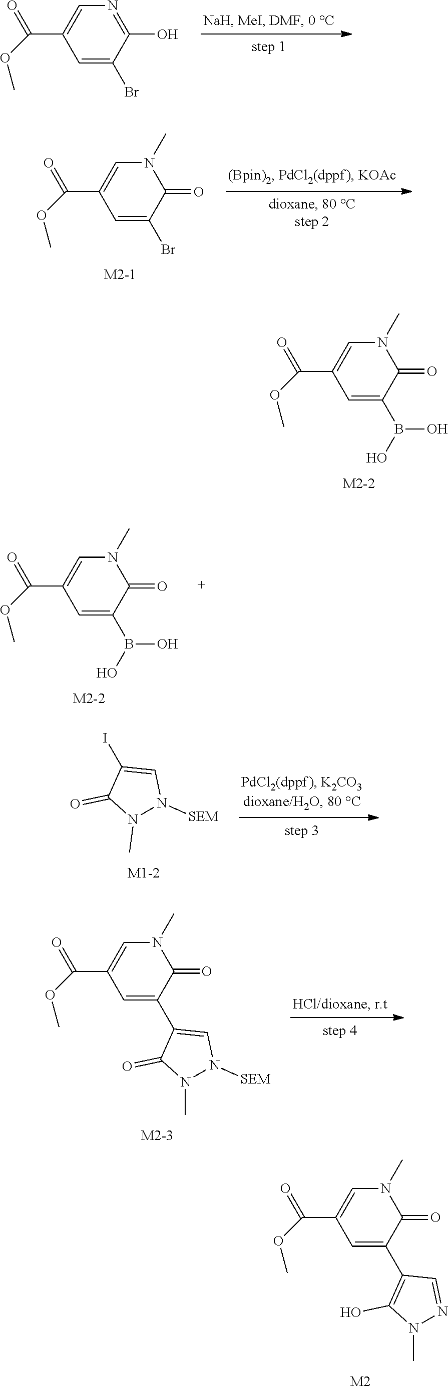

Step 1. Preparation of methyl 5-bromo-1-methyl-6-oxopyridine-3-carboxylate (M2-1). To a mixture of methyl 5-bromo-6-hydroxypyridine-3-carboxylate (20 g, 86.19 mmol, 1 equiv) in N,N-dimethylformamide (100 mL) was added NaH (60%) (4.14 g, 172.39 mmol, 2 equiv) at 0 °C. The mixture was stirred for 60 min at 0 °C under nitrogen atmosphere. CH₃I (13.46 g, 94.81 mmol, 1.1 equiv) was added slowly and the mixture was stirred for 1 h at 0 °C. The reaction was quenched with water (1000 mL) at 0 °C and the mixture was extracted with ethyl acetate (3×500 mL). The combined organic layers were washed with brine (500 mL) and dried over anhydrous Na₂SO₄. After filtration, the filtrate was concentrated under reduced pressure. The residue was purified by trituration with petroleum ether (200 mL) to give 19 g of methyl 5-bromo-1-methyl-6-oxopyridine-3-carboxylate (M2-1) as a white solid (84%). LCMS: m/z (ESI), [M+H]⁺=245.95. ¹H NMR (DMSO-d₆, 400 MHz) δ 3.59 (3H, s), 3.81 (3H, s), 8.18 (1H, d), 8.62 (1H, d).

Step 2. Preparation of (5-(methoxycarbonyl)-1-methyl-2-oxo-1,2-dihydropyridin-3-yl)boronic acid (M2-2). The mixture of methyl 5-bromo-1-methyl-6-oxopyridine-3-carboxylate (30 g, 121.92 mmol, 1 equiv), Pd(dppf)Cl₂—CH₂Cl₂ (8.92 g, 12.2 mmol, 0.1 equiv), bis(pinacolato)diboron (46.44 g, 182.88 mmol, 1.5 equiv), and KOAc (35.90 g, 365.76 mmol, 3 equiv) in 1,4-dioxane (600 mL) was stirred overnight at 80 °C under nitrogen atmosphere. The mixture was cooled to room temperature. The reaction mixture was used in the next step directly without work-up. LCMS: m/z (ESI), [M+H]⁺=212.00.

Step 3. Preparation of methyl 1-methyl-5-(2-methyl-3-oxo-1-{[2-(trimethylsilyl)ethoxy]methyl}pyrazol-4-yl)-6-oxopyridine-3-carboxylate (M2-3). To the mixture, freshly prepared by the reaction described above, was added 4-iodo-2-methyl-1-{[2-(trimethylsilyl)ethoxy]methyl}pyrazol-3-one (43 g, 121.37 mmol, 1.00 equiv), Pd(dppf)Cl₂·CH₂Cl₂ (7.91 g, 9.71 mmol, 0.08 equiv), K₂CO₃ (50.33 g, 364.14 mmol, 3 equiv), and H₂O (150 mL) at room temperature. The mixture was stirred for 2 h at 80 °C under nitrogen atmosphere. The resulting mixture was cooled to room temperature, treated with water (200 mL), and extracted with dichloromethane (3×300 mL). The combined organic layers were washed with brine (500 mL) and dried over anhydrous Na₂SO₄. After filtration, the filtrate was concentrated under reduced pressure. The residue was purified on a silica gel column eluted with petroleum ether/ethyl acetate (1/8) to give 30 g of methyl 1-methyl-5-(2-methyl-3-oxo-1-{[2-(trimethylsilyl)ethoxy]methyl}pyrazol-4-yl)-6-oxopyridine-3-carboxylate (M2-3) as a brown solid (62%). LCMS: m/z (ESI), [M+H]⁺=394.10. ¹H NMR (DMSO-d₆, 400 MHz) δ −0.06 (9H, s), 0.82 (2H, t), 3.37 (3H, s), 3.47 (2H, t), 3.60 (3H, s), 3.81 (3H, s), 5.33 (2H, s), 8.41 (1H, d), 8.85 (1H, s), 9.21 (1H, d).

Step 4. Preparation of methyl 5-(5-hydroxy-1-methylpyrazol-4-yl)-1-methyl-6-oxopyridine-3-carboxylate (M2). A mixture of methyl 1-methyl-5-(2-methyl-3-oxo-1-{[2-(trimethylsilyl)ethoxy]methyl}pyrazol-4-yl)-6-oxopyridine-3-carboxylate (30 g, 76.23 mmol, 1 equiv) and HCl in 1,4-dioxane (50 mL) was stirred overnight at room temperature. The resulting mixture was concentrated under reduced pressure. The residue was taken up with dichloromethane (50 mL) and basified to pH 9 with K₂CO₃. The resulting mixture was filtered and the filter cake was washed with dichloromethane (5×50 mL). The resulting solution was concentrated under reduced pressure. The residue was purified on a silica gel column eluted with dichloromethane/methanol (10/1) to give 15 g of methyl 5-(5-hydroxy-1-methylpyrazol-4-yl)-1-methyl-6-oxopyridine-3-carboxylate (M2) as a brown solid (74%). LCMS: m/z (ESI), [M+H]⁺=264.10. ¹H NMR (DMSO-d₆, 400 MHz) δ 3.52 (3H, s), 3.65 (3H, s), 3.83 (3H, s), 7.76 (1H, d), 8.09 (1H, s), 8.45 (1H, s), 13.34 (1H, s).

### Intermediate M3

### (11R)-16-Bromo-5,11,26-trimethyl-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione

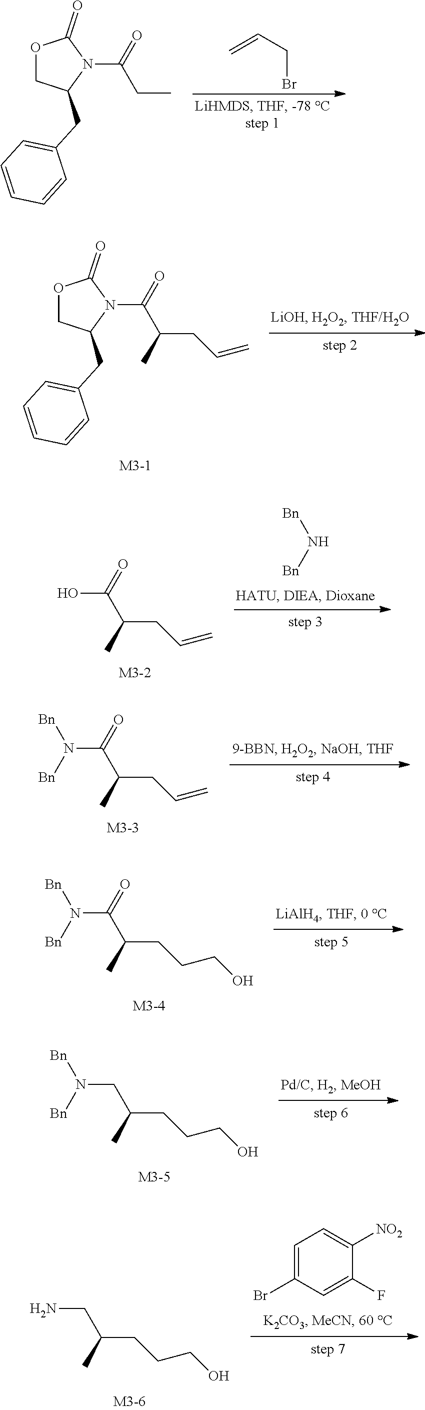

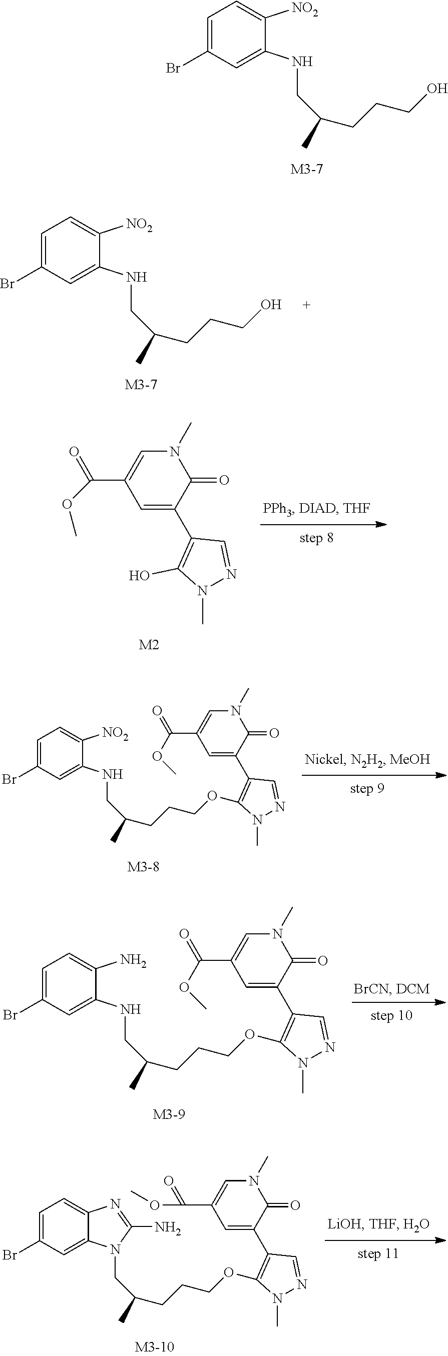

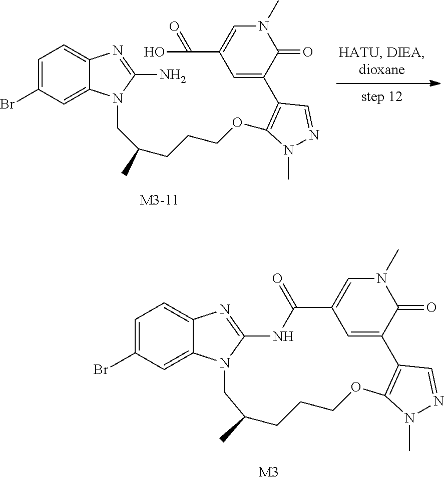 [SMILES: C[C@H](CCCOc1c(-c2cc(C(=O)O)cn(C)c2=O)cnn1C)Cn1c(N)nc2ccc(Br)cc21 ; C[C@@H]1CCCOc2c(cnn2C)-c2cc(cn(C)c2=O)C(=O)Nc2nc3ccc(Br)cc3n2C1 ; "M3"]

Step 1. Preparation of (4S)-4-benzyl-3-[(2R)-2-methylpent-4-enoyl]-1,3-oxazolidin-2-one (M3-1). To a stirred mixture of (4S)-4-benzyl-3-propanoyl-1,3-oxazolidin-2-one (100 g, 428.69 mmol, 1 equiv) in tetrahydrofuran (640 mL) were added LiHMDS (471 mL, 471.56 mmol, 1.1 equiv) and allyl bromide (207.4 g, 1714.77 mmol, 4 equiv) dropwise at −78 °C under nitrogen atmosphere sequentially. The resulting mixture was stirred for 2 h at −50 °C under nitrogen atmosphere and warmed to room temperature. The reaction mixture was treated with sat. NH₄Cl (aq.) solution (1000 mL) and extracted with CH₂Cl₂ (3×400 mL). The combined organic layers were washed with brine (3×500 mL) and dried over anhydrous Na₂SO₄. After filtration, the filtrate was concentrated under reduced pressure. The residue was purified by silica gel column chromatography eluting with petroleum ether/ethyl acetate (40/1) to give 100 g of (4S)-4-benzyl-3-[(2R)-2-methylpent-4-enoyl]-1,3-oxazolidin-2-one (M3-1) as a colorless oil (85%). LCMS: m/z (EST), [M+H]⁺=274.00. ¹H NMR (DMSO-d₆, 400 MHz) δ 1.00-1.12 (3H, t), 2.08-2.49 (2H, m), 2.84-3.06 (2H, m), 3.64-3.78 (1H, m), 4.11-4.26 (1H, m), 4.29-4.39 (1H, m), 4.59-4.75 (1H, m), 4.98-5.18 (2H, m), 5.70-5.87 (1H, m), 7.13-7.38 (5H, m).

Step 2. Preparation of (2R)-2-methylpent-4-enoic acid (M3-2). To a stirred mixture of (4S)-4-benzyl-3-[(2R)-2-methylpent-4-enoyl]-1,3-oxazolidin-2-one (M3-1, 100 g, 365.85 mmol, 1 equiv) in tetrahydrofuran (800 mL) and H₂O (200 mL) were added LiOH·H₂O (46.05 g, 1097.56 mmol, 3 equiv) and H₂O₂(30%) (114.30 mL, 4906 mmol, 13.4 equiv) at 0 °C. The resulting mixture was stirred for 3 h at 0 °C under nitrogen atmosphere. The reaction was quenched with Na₂S₂O₃ solution (250 mL), acidified to pH=1 with HCl solution, and extracted with CH₂Cl₂ (3×400 mL). The combined organic layers were washed with brine (3×500 mL) and dried over anhydrous Na₂SO₄. After filtration, the filtrate was concentrated under reduced pressure. The crude product was used in the next step directly without further purification.

Step 3. Preparation of (2R)—N,N-dibenzyl-2-methylpent-4-enamide (M3-3). To a stirred solution of (2R)-2-methylpent-4-enoic acid (50 g, 219.02 mmol, 1 equiv) and dibenzyl amine (43.2 g, 219.022 mmol, 1 equiv) in dioxane (200 mL) were added N,N-diisopropylethylamine (56.6 g, 438.04 mmol, 2 equiv) and N,N,N,N-tetramethyl-O-(7-azabenzotriazol-1-yl)uronium hexafluorophospate (99.94 g, 262.82 mmol, 1.2 equiv) at room temperature under nitrogen atmosphere. The resulting mixture was stirred for 2 h. The reaction was quenched with water (1000 mL) and the mixture was extracted with CH₂Cl₂ (3×300 mL). The combined organic layers were washed with brine (3×500 mL) and dried over anhydrous Na₂SO₄. After filtration, the filtrate was concentrated under reduced pressure. The residue was purified on silica gel column eluting with petroleum ether/ethyl acetate (40/1) to give 55.6 g of (2R)—N,N-dibenzyl-2-methylpent-4-enamide (M3-3) as a yellow oil (86%). LCMS: m/z (ESI), [M+H]⁺=294.15. ¹H NMR (DMSO-d₆, 400 MHz) δ 1.01 (3H, d), 1.94-2.39 (2H, m), 2.76-2.90 (1H, m), 4.35-4.67 (4H, m), 4.89-5.11 (2H, m), 5.58-5.81 (1H, m), 7.08-7.43 (10H, m).

Step 4. Preparation of (2R)—N,N-dibenzyl-5-hydroxy-2-methylpentanamide (M3-4).

To a stirred mixture of (2R)—N,N-dibenzyl-2-methylpent-4-enamide (50 g, 170.41 mmol, 1 equiv) and 9-borabicyclo[3.3.1]nonane (852.05 mL, 426.02 mmol, 2.5 equiv) in tetrahydrofuran (60 mL) were added NaOH (10 M) (220 mL) and H₂O₂(30%) (151 mL) dropwise at 0 °C. The resulting mixture was stirred for 2 h at room temperature. The reaction was quenched with Na₂S₂O₄ (aq.) solution (500 mL). The mixture was extracted with CH₂Cl₂ (3×500 mL) and the combined organic layers were dried over anhydrous Na₂SO₄. After filtration, the filtrate was concentrated under reduced pressure. The residue was purified on silica gel column eluting with petroleum ether/ethyl acetate (1/1) to give 42.5 g of (2R)—N,N-dibenzyl-5-hydroxy-2-methylpentanamide (M3-4) as a yellow oil (68%). LCMS: m/z (ESI), [M+H]⁺=312.10. ¹H NMR (DMSO-d₆, 400 MHz) δ 1.01 (3H, d), 1.24-1.43 (3H, m), 1.56-1.70 (1H, m), 2.60-2.80 (1H, m), 3.26-3.33 (2H, m), 4.30-4.42 (1H, m), 4.44-4.64 (4H, m), 7.15-7.23 (4H, m), 7.23-7.43 (6H, m).

Step 5. Preparation of (4R)-5-(dibenzylamino)-4-methylpentan-1-ol (M3-5). To a stirred mixture of LiAlH₄ (6.73 g, 177.41 mmol, 1.3 equiv) in tetrahydrofuran (600 mL) was added (2R)—N,N-dibenzyl-5-hydroxy-2-methylpentanamide (42.5 g, 136.46 mmol, 1 equiv) at 0 °C. The resulting mixture was stirred for 2 h at room temperature. The reaction was quenched with water (7 mL) and 15 mL of NaOH (w/w, 30%) at 0 °C and dried over anhydrous Na₂SO₄. After filtration, the filtrate was concentrated under reduced pressure. The residue was purified on silica gel column eluting with petroleum ether/ethyl acetate (10/1) to give 27.9 g of (4R)-5-(dibenzylamino)-4-methylpentan-1-ol (M3-5) as a yellow oil (68%). LCMS: m/z (ESI), [M+H]⁺=298.15. ¹H NMR (DMSO-d₆, 400 MHz) δ 0.81 (3H, d), 0.88-1.00 (1H, m), 1.13-1.46 (3H, m), 1.64-1.78 (1H, m), 2.03-2.27 (2H, m), 3.27-3.39 (2H, m), 3.44 (2H, d), 3.49-3.59 (2H, d), 4.32 (1H, t), 7.18-7.26 (2H, m), 7.30-7.35 (8H, m).

Step 6. Preparation of (4R)-5-amino-4-methylpentan-1-ol (M3-6). A mixture of (4R)-5-(dibenzylamino)-4-methylpentan-1-ol (18 g, 60.51 mmol, 1 equiv) and Pd/C (1.29 g, 12.10 mmol, 0.2 equiv) in methanol (200 mL) was stirred for 6 h at room temperature under hydrogen atmosphere. The resulting mixture was filtered and the filter cake was washed with methanol (3×70 mL). The resulting solution was concentrated under reduced pressure to give 6.5 g of (4R)-5-amino-4-methylpentan-1-ol (M3-6) as a yellow oil (91%). ¹H NMR (DMSO-d₆, 400 MHz) δ 0.83 (3H, d), 0.94-1.09 (2H, m), 1.25-1.54 (3H, m), 2.26-2.49 (2H, m), 3.17 (1H, s), 3.37 (2H, t).

Step 7. Preparation of (4R)-5-[(5-bromo-2-nitrophenyl)amino]-4-methylpentan-1-ol (M3-7). A mixture of 4-bromo-2-fluoro-1-nitrobenzene (10.00 g, 45.46 mmol, 1 equiv), K₂CO₃ (12.56 g, 90.91 mmol, 2 equiv) and (4R)-5-amino-4-methylpentan-1-ol (6.39 g, 54.55 mmol, 1.2 equiv) in acetonitrile (60 mL) was stirred for 2 h at 60 °C under nitrogen atmosphere. The mixture was cooled to room temperature, treated with water (500 mL), and extracted with dichloromethane (3×500 mL). The combined organic layers were washed with brine (3×500 mL) and dried over anhydrous Na₂SO₄. After filtration, the filtrate was concentrated under reduced pressure and purified by prep-TLC eluting with petroleum ether/ethyl acetate (1/3) to give 11.7 g of (4R)-5-[(5-bromo-2-nitrophenyl)amino]-4-methylpentan-1-ol (M3-7) as a yellow solid (81%). LCMS: m/z (ESI), [M+H]⁺=319.00. ¹H NMR (400 MHz, DMSO-d₆) δ 0.95 (3H, d), 1.14-1.27 (1H, m), 1.37-1.59 (3H, m), 1.77-1.88 (1H, m), 3.18-3.22 (1H, m), 3.24-3.34 (1H, m), 3.35-3.44 (2H, m), 4.39 (1H, t), 6.83-6.87 (1H, m), 7.26 (1H, d), 7.99 (1H, d), 8.20 (1H, t).

Step 8. Preparation of methyl 5-(5-{[(4R)-5-[(5-bromo-2-nitrophenyl)amino]-4-methylpentyl]oxy}-1-methylpyrazol-4-yl)-1-methyl-6-oxopyridine-3-carboxylate (M3-8). To a stirred mixture of methyl 5-(5-hydroxy-1-methylpyrazol-4-yl)-1-methyl-6-oxopyridine-3-carboxylate (M2, 2.67 g, 10.09 mmol, 1 equiv) and triphenylphosphine (6.62 g, 25.22 mmol, 2.5 equiv) in tetrahydrofuran (60 mL) were added diisopropylazodicarboxylate (4.08 g, 20.18 mmol, 2 equiv) and (4R)-5-[(5-bromo-2-nitrophenyl)amino]-4-methylpentan-1-ol (3.21 g, 10.09 mmol, 1 equiv) at 0 °C under nitrogen atmosphere. After being stirred for 5 h at room temperature, the mixture was treated with water (500 mL) and extracted with dichloromethane (3×500 mL). The combined organic layers were washed with brine (3×500 mL) and dried over anhydrous Na₂SO₄. After filtration, the filtrate was concentrated under reduced pressure. The residue was purified by prep-TLC eluted with petroleum ether/ethyl acetate (1/4) to give 2.72 g of methyl 5-(5-{[(4R)-5-[(5-bromo-2-nitrophenyl)amino]-4-methylpentyl]oxy}-1-methylpyrazol-4-yl)-1-methyl-6-oxopyridine-3-carboxylate (M3-8) as a yellow solid (48%). LCMS: m/z (EST), [M+H]⁺=564.10. ¹H NMR (400 MHz, DMSO-d₆) δ 0.97 (3H, d), 1.45-1.29 (1H, m), 1.60-1.64 (1H, m), 1.78-1.82 (1H, m), 1.86 (2H, s), 3.38-3.15 (2H, m), 3.58 (3H, s), 3.68 (3H, s), 3.78 (3H, s), 3.96 (2H, t), 6.83-6.88 (1H, m), 7.26 (1H, d), 8.02-7.95 (2H, m), 8.14 (1H, d), 8.21 (1H, t), 8.43 (1H, d).

Step 9. Preparation of methyl 5-(5-{[(4R)-5-[(2-amino-5-bromophenyl)amino]-4-methylpentyl]oxy}-1-methylpyrazol-4-yl)-1-methyl-6-oxopyridine-3-carboxylate (M3-9). To a stirred mixture of methyl 5-(5-{[(4R)-5-[(5-bromo-2-nitrophenyl)amino]-4-methylpentyl]oxy}-1-methylpyrazol-4-yl)-1-methyl-6-oxopyridine-3-carboxylate (4.00 g, 7.11 mmol, 1 equiv) and Raney Ni (200 mg, 2.33 mmol, 0.33 equiv) in methanol (40 mL) was added NH₂NH₂·H₂O (712 mg, 14.22 mmol, 2 equiv) at 0 °C under nitrogen atmosphere.

After being stirred for 3 h at room temperature, the mixture was filtered and the filter cake was washed with methanol (3×200 mL). The organic solution was concentrated under reduced pressure and the residue was purified by prep-TLC eluted dichloromethane/methanol (30/1) to give 3.65 g of methyl 5-(5-{[(4R)-5-[(2-amino-5-bromophenyl)amino]-4-methylpentyl]oxy}-1-methylpyrazol-4-yl)-1-methyl-6-oxopyridine-3-carboxylate (M3-9) as a yellow solid (96%). LCMS: m/z (ESI), [M+H]⁺=533.95.

Step 10. Preparation of methyl 5-(5-{[(4R)-4-[(2-amino-6-bromo-1,3-benzodiazol-1-yl)methyl]pentyl]oxy}-1-methylpyrazol-4-yl)-1-methyl-6-oxopyridine-3-carboxylate (M3-10). To a stirred mixture of methyl 5-(5-{[(4R)-5-[(2-amino-5-bromophenyl)amino]-4-methylpentyl]oxy}-1-methylpyrazol-4-yl)-1-methyl-6-oxopyridine-3-carboxylate (3.00 g, 5.63 mmol, 1 equiv) in dichloromethane (40 mL) was added cyanogen bromide (895 mg, 8.45 mmol, 1.5 equiv) at room temperature. After being stirred for 2 h at room temperature, the mixture was concentrated under reduced pressure. The residue was purified by prep-TLC eluted with dichloromethane/methanol (20/1) to give 2.6 g of methyl 5-(5-{[(4R)-4-[(2-amino-6-bromo-1,3-benzodiazol-1-yl)methyl]pentyl]oxy}-1-methylpyrazol-4-yl)-1-methyl-6-oxopyridine-3-carboxylate (M3-10) as a reddish solid (83%). LCMS: m/z (ESI), [M+H]⁺=559.05.

Step 11. Preparation of 5-(5-{[(4R)-4-{[2-amino-6-(4-methylpiperazin-1-yl)-1,3-benzodiazol-1-yl]methyl}pentyl]oxy}-1-methylpyrazol-4-yl)-1-methyl-6-oxopyridine-3-carboxylic acid (M3-11). To a stirred mixture of methyl 5-(5-{[(4R)-4-{[2-amino-6-(4-methylpiperazin-1-yl)-1,3-benzodiazol-1-yl]methyl}pentyl]oxy}-1-methylpyrazol-4-yl)-1-methyl-6-oxopyridine-3-carboxylate (3.71 g, 6.42 mmol, 1 equiv) in tetrahydrofuran (100 mL) was added LiOH (307 mg, 12.83 mmol, 2 equiv) in H₂O (20 mL) at room temperature. After being stirred for 1 h at room temperature, the reaction mixture was concentrated under reduced pressure. The residue was purified by reverse flash chromatography (10% to 40% acetonitrile in water) to give 3.5 g of 5-(5-{[(4R)-4-{[2-amino-6-(4-methylpiperazin-1-yl)-1,3-benzodiazol-1-yl]methyl}pentyl]oxy}-1-methylpyrazol-4-yl)-1-methyl-6-oxopyridine-3-carboxylic acid (M3-11) as a white solid (97%). LCMS: m/z (ESI), [M+H]⁺=545.15.

Step 12. Preparation of (11R)-16-bromo-5,11,26-trimethyl-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (M3). To a stirred mixture of 5-(5-{[(4R)-4-[(2-amino-6-bromo-1,3-benzodiazol-1-yl)methyl]pentyl]oxy}-1-methylpyrazol-4-yl)-1-methyl-6-oxopyridine-3-carboxylic acid (1.2 g, 2.20 mmol, 1 equiv) in dioxane (18 mL) were added N,N-diisopropylethyl amine (0.85 g, 6.62 mmol, 3 equiv) and N,N,N,N-tetramethyl-O-(7-azabenzotriazol-1-yl)uronium hexafluorophospate (1.26 g, 3.31 mmol, 1.5 equiv) at room temperature. After being stirred for 3 h at 60 °C under nitrogen atmosphere, the reaction mixture was cooled to room temperature, treated with water (300 mL), and extracted with CH₂Cl₂ (2×200 mL). The combined organic layers were washed with brine (3×300 mL) and dried over anhydrous Na₂SO₄. After filtration, the filtrate was concentrated under reduced pressure. The residue was purified by prep-TLC eluted with CH₂Cl₂/methanol (40/1) to give 1.14 g of (11R)-16-bromo-5,11,26-trimethyl-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (M3) as a white solid (98%). LCMS: m/z (ESI), [M+H]⁺=525.15. ¹H NMR (DMSO-d₆, 400 MHz) δ 0.81 (3H, d), 1.43 (1H, q), 1.91 (2H, d), 2.06-2.26 (1H, m), 2.78 (1H, br s), 3.62 (3H, s), 3.72 (3H, s), 3.89-4.00 (2H, m), 4.07-4.15 (1H, m), 4.31-4.38 (1H, m), 7.36 (1H, dd), 7.46 (1H, d), 7.88 (1H, d), 8.30 (1H, d), 8.35 (1H, s), 8.79 (1H, d), 12.67 (1H, s).

### Intermediate M4A and M4B

### 5-Bromo-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, isomer 1 (M4A) and isomer 2 (M4B)

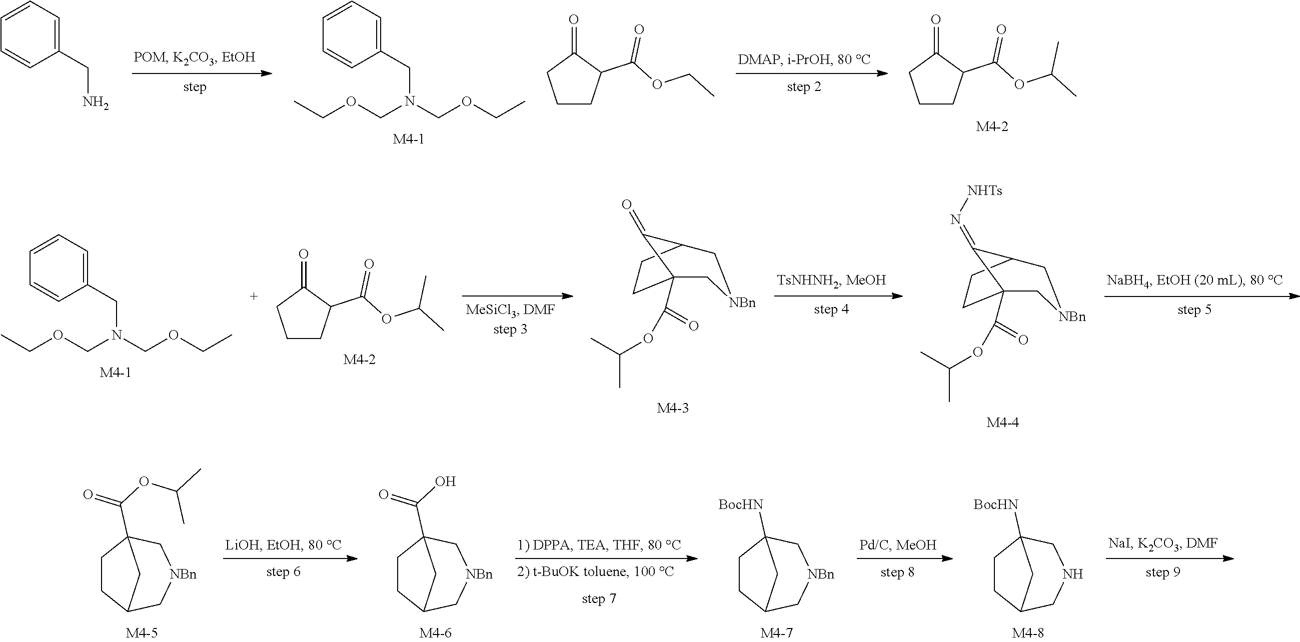

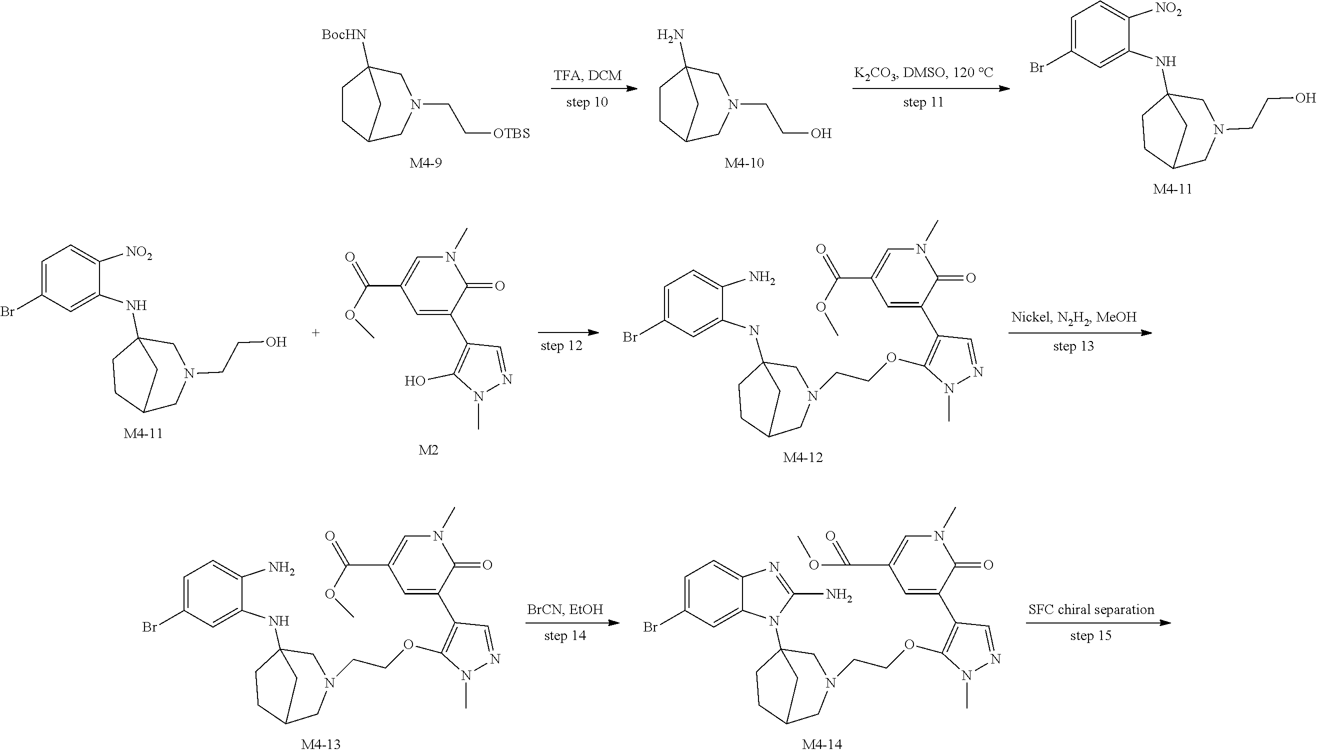

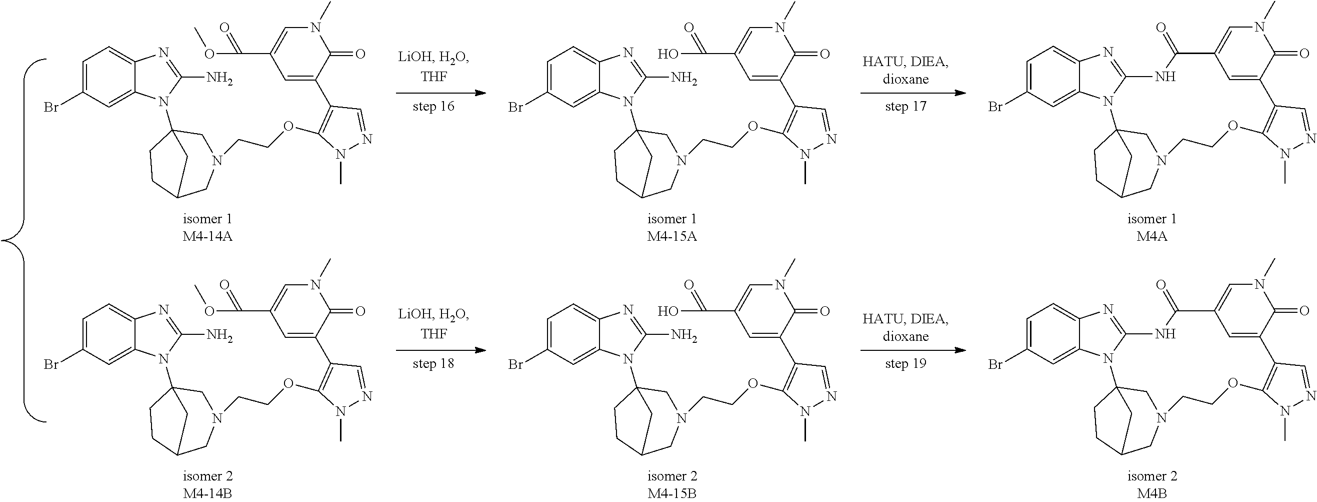 [SMILES: COC(=O)c1cc(-c2cnn(C)c2OCCN2CC3CCC(n4c(N)nc5ccc(Br)cc54)(C3)C2)c(=O)n(C)c1 ; Cn1ncc(-c2cc(C(=O)O)cn(C)c2=O)c1OCCN1CC2CCC(n3c(N)nc4ccc(Br)cc43)(C2)C1 ; Cn1ncc2c1OCCN1CC3CCC(C3)(C1)n1c(nc3ccc(Br)cc31)NC(=O)c1cc-2c(=O)n(C)c1 ; COC(=O)c1cc(-c2cnn(C)c2OCCN2CC3CCC(n4c(N)nc5ccc(Br)cc54)(C3)C2)c(=O)n(C)c1 ; Cn1ncc(-c2cc(C(=O)O)cn(C)c2=O)c1OCCN1CC2CCC(n3c(N)nc4ccc(Br)cc43)(C2)C1 ; Cn1ncc2c1OCCN1CC3CCC(C3)(C1)n1c(nc3ccc(Br)cc31)NC(=O)c1cc-2c(=O)n(C)c1]

Step 1. Preparation of benzylbis(ethoxymethyl)amine (M4-1). A mixture of benzylamine (500 g, 4666 mmol, 1 equiv), POM (840.62 g, 9332 mmol, 2 equiv) and K₂CO₃ (644.88 g, 4666 mmol, 1 equiv) in ethanol (1200 mL) was stirred for 16 h at room temperature under nitrogen atmosphere. The resulting mixture was filtered. The filter cake was washed with dichloromethane (3×200 mL) and organic solution was concentrated under reduced pressure to give 1200 g of benzylbis(ethoxymethyl)amine (M4-1) as a yellow oil (80%). This crude material was used for step 3 without further purification. ¹H NMR (DMSO-d₆, 400 MHz) δ 1.01-1.19 (6H, m), 3.39-3.57 (3H, m), 3.90 (2H, t), 4.55-4.82 (4H, m), 7.27-7.39 (5H, m).

Step 2. Preparation of isopropyl 2-oxocyclopentane-1-carboxylate (M4-2). A mixture of ethyl 2-oxocyclopentane-1-carboxylate (400 g, 2561 mmol, 1 equiv) and DMAP (31.29 g, 256.11 mmol, 0.1 equiv) in i-PrOH (500 mL) was stirred overnight at 80 °C under nitrogen atmosphere. The mixture was cooled to room temperature and concentrated under reduced pressure. The residue was purified on silica gel column eluted with petroleum ether/ethyl acetate (10/1) to give 400 g of isopropyl 2-oxocyclopentane-1-carboxylate (M4-2) as a yellow oil (92%). LCMS: m/z (EST), [M−H]⁺=168.85. ¹H NMR (DMSO-d₆, 400 MHz) δ 1.26 (6H, t), 1.80-1.92 (1H, m), 2.07-2.17 (1H, m), 2.25-2.35 (4H, m), 3.11 (1H, t), 5.00-5.10 (1H, m).

Step 3. Preparation of isopropyl 3-benzyl-8-oxo-3-azabicyclo[3.2.1]octane-1-carboxylate (M4-3). A solution of benzylbis(methoxymethyl)amine (M4-1, 601.1 g, 3078.5 mmol, 2.62 equiv) in DMF (1.5 L) was treated with methyltrichlorosilane (400.4 g, 2679 mmol, 2.28 equiv) for 10 min at 0 °C under nitrogen atmosphere followed by the addition of isopropyl 2-oxocyclopentane-1-carboxylate (M4-2, 200 g, 1175 mmol, 1 equiv) slowly at 0 °C. The resulting mixture was stirred overnight at room temperature under nitrogen atmosphere. The reaction was quenched with water (3 L), basified to pH 7 with saturated NaHCO₃ (aq.) solution (3 L) and the mixture was extracted with ethyl acetate (3×2 L). The combined organic layers were washed with brine (3×2 L), dried over anhydrous Na₂SO₄, and concentrated under reduced pressure. The residue was purified on silica gel column eluted with petroleum ether/ethyl acetate (40/1) and concentrated under vacuum. The residue was further purified by Prep-HPLC by reverse flash chromatography with C18 silica gel column. Fractions containing the desired compound were evaporated to dryness to give 110 g of isopropyl 3-benzyl-8-oxo-3-azabicyclo[3.2.1]octane-1-carboxylate (M4-3) as a yellow oil (31%). LCMS: m/z (ESI), [M+H]⁺=470.15. ¹H NMR (DMSO-d₆, 400 MHz) δ 1.04-1.26 (6H, m), 1.84-1.96 (2H, m), 2.16-2.37 (3H, m), 2.45 (1H, d), 2.62 (1H, d), 2.94 (1H, d), 3.04 (1H, d), 3.65 (2H, s), 4.75-5.02 (1H, m), 7.13-7.48 (5H, m)

Step 4. Preparation of isopropyl 3-benzyl-8-[(4-methylbenzenesulfonamido)imino]-3-azabicyclo[3.2.1]octane-1-carboxylate (M4-4). A solution of isopropyl 3-benzyl-8-oxo-3-azabicyclo[3.2.1]octane-1-carboxylate (100 g, 331.8 mmol, 1 equiv) in methanol (1 L) under nitrogen atmosphere followed by the addition of TsNHNH₂ (123.58 g, 663.5 mmol, 2.00 equiv) in portions at 0 °C. The resulting mixture was stirred overnight at room temperature under nitrogen atmosphere. The resulting mixture was concentrated under reduced pressure. The residue was purified on silica gel column and eluted with petroleum ether/ethyl acetate (5/1) to give 80 g of isopropyl 3-benzyl-8-[(4-methylbenzenesulfonamido)imino]-3-azabicyclo[3.2.1]octane-1-carboxylate (M4-4) as a white solid (51%). ¹H NMR (DMSO-d₆, 400 MHz) δ 0.99-1.15 (6H, m), 1.57-1.85 (2H, m), 1.87-2.03 (1H, ddd), 2.02-2.13 (2H, m), 2.25 (1H, d), 2.39 (3H, s), 2.62-2.73 (1H, m), 2.84 (1H, d) 3.14-3.19 (1H, m), 3.06-3.10 (1H, m), 3.50 (2H, s), 4.75-4.91 (1H, m), 7.26-7.43 (7H, m), 7.67 (2H, d), 10.38 (1H, s).

Step 5. Preparation of 3-benzyl-3-azabicyclo[3.2.1]octane-1-carboxylate (M4-5). To a stirred solution of isopropyl 3-benzyl-8-[(4-methylbenzenesulfonamido)imino]-3-azabicyclo[3.2.1]octane-1-carboxylate (100 g, 212.95 mmol, 1 equiv) in methanol (2 L) was added NaBH₄ (120.8 g, 3194.20 mmol, 15 equiv) at 0 °C under nitrogen atmosphere. The mixture was stirred overnight at 80 °C and then cooled to room temperature. The reaction was quenched with water (1 L) and the mixture was extracted with ethyl acetate (3×1 L). The combined organic layers were washed with brine (3×1 L), dried over anhydrous Na₂SO₄, and concentrated under reduced pressure. The residue was purified by silica gel chromatography and eluted with petroleum ether/ethyl acetate (5/1) to give 33.0 g of isopropyl 3-benzyl-3-azabicyclo[3.2.1]octane-1-carboxylate (M4-5) as a colorless oil (54%). LCMS: m/z (ESI), [M+H]⁺=288.05. ¹H NMR (DMSO-d₆, 400 MHz) δ 1.14 (6H, d), 1.41 (1H, d), 1.62-1.69 (2H, m), 1.70-1.90 (3H, m), 1.9-2.04 (1H, m), 2.12 (1H, d), 2.18 (1H, d), 2.58 (1H, d), 2.82 (1H, d), 3.50 (2H, d), 4.81-4.90 (1H, m), 7.06-7.51 (5H, m).

Step 6. Preparation of 3-benzyl-3-azabicyclo[3.2.1]octane-1-carboxylic acid (M4-6). A mixture of isopropyl 3-benzyl-3-azabicyclo[3.2.1]octane-1-carboxylate (33.0 g, 114.82 mmol, 1 equiv) and LiOH (3.0 g, 126.30 mmol, 1.1 equiv) in ethanol (400 mL) was stirred for 2 days at 80 °C under nitrogen atmosphere. The mixture was cooled to room temperature and concentrated under reduced pressure to give 30 g of 3-benzyl-3-azabicyclo[3.2.1]octane-1-carboxylic acid (M4-6) as a yellow oil (96%). LCMS: m/z (EST), [M+H]⁺=246.05.

Step 7. Preparation of tert-butyl N-{3-benzyl-3-azabicyclo[3.2.1]octan-1-yl}carbamate (M4-7). A mixture of 3-benzyl-3-azabicyclo[3.2.1]octane-1-carboxylic acid (30 g, 110.06 mmol, 1 equiv, 90%), DPPA (36.35 g, 132.07 mmol, 1.2 equiv) and triethylamine (33.41 g, 330.18 mmol, 3.0 equiv) in tetrahydrofuran (300 mL) was stirred for 2 h at 80 °C under nitrogen atmosphere. The mixture was cooled to room temperature and concentrated under reduced pressure. Toluene (500 mL) and t-BuOK (18.53 g, 165.09 mmol, 1.5 equiv) was added at 0 °C. The mixture was stirred for 2 h at 100 °C under nitrogen atmosphere and then cooled to room temperature. The reaction was quenched with water (1 L) and the mixture was extracted with ethyl acetate (3×1 L). The combined organic layers were washed with brine (3×1 L), dried over anhydrous Na₂SO₄, and concentrated under reduced pressure. The residue was purified by silica gel chromatography eluted with petroleum ether/ethyl acetate (5/1) to give 15 g of tert-butyl N-{3-benzyl-3-azabicyclo[3.2.1]octan-1-yl}carbamate (M4-7) as a white solid (43%). LCMS: m/z (ESI), [M+H]⁺=317.15. ¹H NMR (DMSO-d₆, 400 MHz) δ 1.23 (9H, s), 1.38-1.60 (3H, m), 1.59-1.76 (2H, m), 1.75-2.25 (5H, m), 2.87 (1H, d), 3.38-3.56 (2H, m), 6.88 (1H, s), 7.17-7.37 (5H, m)

Step 8. Preparation of tert-butyl N-{3-azabicyclo[3.2.1]octan-1-yl}carbamate (M4-8). A mixture of tert-butyl N-{3-benzyl-3-azabicyclo[3.2.1]octan-1-yl}carbamate (15 g, 47.40 mmol, 1 equiv) and Pd/C (7.57 g, 71.13 mmol, 1.50 equiv) in methanol (300 mL) was stirred for 2 h at room temperature under hydrogen atmosphere. The resulting mixture was filtered and the filter cake was washed with methanol (3×200 mL). The organic solution was concentrated under reduced pressure to give 10 g of tert-butyl N-{3-azabicyclo[3.2.1]octan-1-yl}carbamate (M4-8) as a white solid (93%). This material was used for the next step without further purification. LCMS: m/z (ESI), [M+H]⁺=227.10. ¹H NMR (DMSO-d₆, 400 MHz) δ 1.36 (9H, d), 1.45-1.60 (2H, m), 1.60-1.75 (1H, m), 1.73-1.91 (1H, m), 1.93-2.00 (2H, m), 2.45 (2H, d), 2.76-2.89 (1H, m), 3.14-3.20 (1H, m), 6.73 (1H, d).

Step 9. Preparation of tert-butyl N-(3-{2-[(tert-butyldimethylsilyl)oxy]ethyl}-3-azabicyclo[3.2.1]octan-1-yl)carbamate (M4-9). A mixture of freshly prepared tert-butyl N-{3-azabicyclo[3.2.1]octan-1-yl}carbamate (10 g, ~44 mmol, ~1 equiv), (2-bromoethoxy)(tert-butyl)dimethylsilane (21.14 g, 88.37 mmol, 2 equiv), NaI (13.25 g, 88.37 mmol, 2 equiv) and K₂CO₃ (12.21 g, 88.37 mmol, 2 equiv) in DMF (160 mL) was stirred for 2 h at 60 °C under nitrogen atmosphere. The mixture was cooled to room temperature. The reaction was quenched with water (300 mL) and the mixture was extracted with ethyl acetate (3×200 mL). The combined organic layers were washed with brine (3×300 mL), dried over anhydrous Na₂SO₄, and concentrated under reduced pressure. The residue was purified by prep-HPLC using water containing 0.1% NH₄HCO₃ and acetonitrile as mobile phase. Fractions containing the desired compound were evaporated to dryness to give 12 g of tert-butyl N-(3-{2-[(tert-butyldimethylsilyl)oxy]ethyl}-3-azabicyclo[3.2.1]octan-1-yl)carbamate (M4-9) as a colorless oil (70%). LCMS: m/z (ESI), [M+H]⁺=385.25. ¹H NMR (DMSO-d₆, 400 MHz) δ 0.00 (6H, s), 0.82 (9H, s), 0.85 (1H, s), 1.32 (9H, s), 1.37-1.48 (2H, m), 1.53-1.64 (2H, m), 1.91-2.05 (4H, m), 2.36-2.43 (2H, m), 2.54-2.57 (1H, m), 2.86-2.92 (1H, m), 3.61 (2H, t), 6.80 (1H, s).

Step 10. Preparation of 2-(1-amino-3-azabicyclo[3.2.1]octan-3-yl)ethan-1-ol (M4-10). A mixture of tert-butyl N-(3-{2-[(tert-butyldimethylsilyl)oxy]ethyl}-3-azabicyclo[3.2.1]octan-1-yl)carbamate (12 g, 31.19 mmol, 1 equiv) in trifluoroacetic acid (28 mL) and dichloromethane (84 mL) was stirred for 2 h at room temperature under nitrogen atmosphere. The resulting mixture was concentrated under reduced pressure. The crude product of 2-(1-amino-3-azabicyclo[3.2.1]octan-3-yl)ethan-1-ol (M4-10) as an orange oil was used in the next step directly without further purification. LCMS: m/z (ESI), [M+H]⁺=285.15.

Step 11. Preparation of 2-[1-[(5-bromo-2-nitrophenyl)amino]-3-azabicyclo[3.2.1]octan-3-yl]ethanol (M4-11). A mixture of 2-(1-amino-3-azabicyclo[3.2.1]octan-3-yl)ethan-1-ol (crude, M4-10), K₂CO₃ (21.56 g, 156.25 mmol, 5 equiv) and 4-bromo-2-fluoro-1-nitrobenzene (20.53 g, 93.73 mmol, 3 equiv) in DMSO (160 mL) was stirred for 2 h at 120 °C under nitrogen atmosphere. The mixture was cooled to room temperature. The reaction was quenched with water (300 mL) and the mixture was extracted with ethyl acetate (3×200 mL). The combined organic layers were washed with brine (3×300 mL), dried over anhydrous Na₂SO₄, and concentrated under reduced pressure. The crude product was purified by prep-HPLC using water containing 0.10% NH₄HCO₃ and acetonitrile as mobile phase. Fractions containing the desired compound were evaporated to dryness to give 12 g of 2-[1-[(5-bromo-2-nitrophenyl)amino]-3-azabicyclo[3.2.1]octan-3-yl]ethanol (M4-11) as an orange semi-solid (76%). LCMS: m/z (ESI), [M+H]⁺=371.95. ¹H NMR (DMSO-d₆, 400 MHz) δ 1.68 (3H, d), 1.82-1.85 (1H, m), 1.94 (1H, s), 2.12 (1H, d), 2.17 (1H, d), 2.21 (1H, s), 2.36 (1H, s), 2.49 (2H, d), 2.65-2.73 (1H, m), 3.10-3.20 (1H, m), 3.47-3.57 (2H, m), 4.36 (1H, t), 6.86-3.89 (1H, m), 7.21 (1H, d), 8.01 (1H, d), 8.31 (1H, s).

Step 12. Preparation of methyl 5-[5-(2-{1-[(5-bromo-2-nitrophenyl)amino]-3-azabicyclo[3.2.1]octan-3-yl}ethoxy)-1-methylpyrazol-4-yl]-1-methyl-6-oxopyridine-3-carboxylate (M4-12). To a stirred mixture of 2-{1-[(5-bromo-2-nitrophenyl)amino]-3-azabicyclo[3.2.1]octan-3-yl}ethanol (8 g, 21.61 mmol, 1 equiv) and triphenylphosphine (17.00 g, 64.82 mmol, 3 equiv) in tetrahydrofuran (500 mL) were added diisopropylazodicarboxylate (12.23 g, 60.50 mmol, 2.8 equiv) and methyl 5-(5-hydroxy-1-methylpyrazol-4-yl)-1-methyl-6-oxopyridine-3-carboxylate (5.69 g, 21.61 mmol, 1 equiv) slowly at 0 °C under nitrogen atmosphere. The resulting mixture was stirred for 2 h at 0 °C under nitrogen atmosphere. The reaction was quenched with water (300 mL) and the mixture was extracted with ethyl acetate (3×200 mL). The combined organic layers were washed with brine (3×300 mL), dried over anhydrous Na₂SO₄, and concentrated under reduced pressure. The residue was purified on a silica gel column eluted with petroleum ether/ethyl acetate (1/3) to give 9.5 g of methyl 5-[5-(2-{1-[(5-bromo-2-nitrophenyl)amino]-3-azabicyclo[3.2.1]octan-3-yl}ethoxy)-1-methylpyrazol-4-yl]-1-methyl-6-oxopyridine-3-carboxylate (M4-12) as an orange oil (71%). LCMS: m/z (ESI), [M+H]⁺=617.10.

Step 13. Preparation of methyl 5-[5-(2-{1-[(2-amino-5-bromophenyl)amino]-3-azabicyclo[3.2.1]octan-3-yl}ethoxy)-1-methylpyrazol-4-yl]-1-methyl-6-oxopyridine-3-carboxylate (M4-13). To a mixture of methyl 5-[5-(2-{l-[(5-bromo-2-nitrophenyl)amino]-3-azabicyclo[3.2.1]octan-3-yl}ethoxy)-1-methylpyrazol-4-yl]-1-methyl-6-oxopyridine-3-carboxylate (10.5 g, 17.06 mmol, 1 equiv) and Raney-Ni (2 g) in methanol (150 mL) was added hydrazine hydrate (2.19 g, 68.24 mmol, 1.5 equiv) and stirred for 2 h at 0 °C under nitrogen atmosphere. The resulting mixture was filtered and the filter cake was washed with dichloromethane (3×200 mL). The organic solution was concentrated under reduced pressure. The residue was purified on a silica gel column eluted with dichloromethane/methanol (10/1) to give 9.5 g of methyl 5-[5-(2-{1-[(2-amino-5-bromophenyl)amino]-3-azabicyclo[3.2.1]octan-3-yl}ethoxy)-1-methylpyrazol-4-yl]-1-methyl-6-oxopyridine-3-carboxylate (M4-13) as a brown oil (91%). LCMS: m/z (ESI), [M+H]⁺=585.20. ¹H NMR (DMSO-d₆, 400 MHz) δ 1.45-1.60 (3H, m), 1.70-1.85 (2H, m), 1.95-2.17 (4H, m), 2.65-2.80 (3H, m), 3.09-3.15 (1H, m), 3.17 (2H, d), 3.57 (3H, s), 3.74 (3H, s), 3.79 (3H, s), 4.00-4.10 (3H, m), 4.52 (1H, s), 4.75 (2H, s), 6.44-6.50 (1H, m), 6.50-6.56 (1H, m), 6.64 (1H, d), 7.97 (1H, s), 8.10 (1H, d), 8.43 (1H, d).

Step 14. Preparation of methyl 5-(5-{2-[1-(2-amino-6-bromo-1,3-benzodiazol-1-yl)-3-azabicyclo[3.2.1]octan-3-yl]ethoxy}-1-methylpyrazol-4-yl)-1-methyl-6-oxopyridine-3-carboxylate (M4-14). A solution of methyl 5-[5-(2-{1-[(2-amino-5-bromophenyl)amino]-3-azabicyclo[3.2.1]octan-3-yl}ethoxy)-1-methylpyrazol-4-yl]-1-methyl-6-oxopyridine-3-carboxylate (10 g, 17.08 mmol, 1 equiv) and cyanogen bromide (2.17 g, 20.49 mmol, 1.20 equiv) in ethanol (200 mL) was stirred overnight at room temperature under nitrogen atmosphere. The resulting mixture was concentrated under reduced pressure and the residue was purified on a silica gel column eluted with dichloromethane/methanol (20/1) to give 9.5 g of methyl 5-(5-{2-[1-(2-amino-6-bromo-1,3-benzodiazol-1-yl)-3-azabicyclo[3.2.1]octan-3-yl]ethoxy}-1-methylpyrazol-4-yl)-1-methyl-6-oxopyridine-3-carboxylate (M4-14) as a brown solid (91%). LCMS: m/z (ESI), [M+H]⁺=612.05.

Step 15. Preparation of methyl 5-(5-{2-[1-(2-amino-6-bromo-1,3-benzodiazol-1-yl)-3-azabicyclo[3.2.1]octan-3-yl]ethoxy}-1-methylpyrazol-4-yl)-1-methyl-6-oxopyridine-3-carboxylate, isomer 1 (M4-14A) and isomer 2 (M4-14B). A racemic mixture of M4-14 (9 g, 14.74 mmol) was separated by chiral chromatography using (R, R)-WHELK-01-Kromasil column (5*25 cm, 5 μm), CO₂ as mobile phase A, and methanol/acetonitrile (1/1) as moble phase B to afford 3.6 g of isomer 1 (M4-14A,41%) and 3.9 g of isomer 2 (M4-14B, 43%) as a brown solid. SFC-HPLC, Rt (isomer 1)=2.246 min, Rt (isomer 2)=3.898 min.

Step 16. Preparation of 5-(5-(2-(1-(2-amino-6-bromo-1H-benzo[d]imidazol-1-yl)-3-azabicyclo[3.2.1]octan-3-yl)ethoxy)-1-methyl-1H-pyrazol-4-yl)-1-methyl-6-oxo-1,6-dihydropyridine-3-carboxylic acid (isomer 1, M4-15A). To a stirred solution of methyl 5-(5-(2-(1-(2-amino-6-bromo-1H-benzo[d]imidazol-1-yl)-3-azabicyclo[3.2.1]octan-3-yl)ethoxy)-1-methyl-1H-pyrazol-4-yl)-1-methyl-6-oxo-1,6-dihydropyridine-3-carboxylate (isomer 1, M4-14A, 4 g, 6.55 mmol, 1 equiv) in tetrahydrofuran (8 mL) was added LiOH (0.31 g, 13.10 mmol, 2 equiv) in H₂O (2 mL). The resulting mixture was stirred for 2 h at room temperature. The reaction was concentrated under reduced pressure and the residue was purified by Prep-HPLC by reverse flash chromatography with C18 silica gel column eluted with water and acetonitrile. Fractions containing the desired compound were evaporated to dryness to give 3.6 g of 5-(5-(2-(1-(2-amino-6-bromo-1H-benzo[d]imidazol-1-yl)-3-azabicyclo[3.2.1]octan-3-yl)ethoxy)-1-methyl-1H-pyrazol-4-yl)-1-methyl-6-oxo-1,6-dihydropyridine-3-carboxylic acid (isomer 1, M4-15A) as a white solid (92%). LCMS: m/z (ESI), [M+H]⁺=598.05.

Step 17. Preparation of 5-bromo-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (M4A). A mixture of 5-(5-{2-[1-(2-amino-6-bromo-1,3-benzodiazol-1-yl)-3-azabicyclo[3.2.1]octan-3-yl]ethoxy}-1-methylpyrazol-4-yl)-1-methyl-6-oxopyridine-3-carboxylic acid (M4-15A, 3.6 g, 6.03 mmol, 1 equiv), N,N,N,N-tetramethyl-O-(7-azabenzotriazol-1-yl)uronium hexafluorophospate (3.44 g, 9.05 mmol, 1.5 equiv), and N,N-diisopropylethylamine (2.34 g, 18.10 mmol, 3 equiv) in 1,4-dioxane (80 mL) was stirred for 2 h at room temperature under nitrogen atmosphere. The reaction was quenched with water (300 mL) and the mixture was extracted with ethyl acetate (3×200 mL). The combined organic layers were washed with brine (3×300 mL), dried over anhydrous Na₂SO₄, and concentrated under reduced pressure. The residue was purified on a silica gel column eluted with dichloromethane/methanol (20/1) to give 3.3 g of 5-bromo-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (isomer 1, M4A) as a brown soild (94%). LCMS: m/z (EST), [M+H]⁺=580.00. SFC-HPLC, Rt=5.498, ¹H NMR (DMSO-d₆, 400 MHz) δ 1.56-1.81 (3H, m), 2.01 (1H, d), 2.42 (2H, d), 2.54 (1H, d), 2.63 (1H, s), 2.69-2.75 (1H, m), 3.09-3.20 (2H, m), 3.19-3.30 (1H, m), 3.63 (3H, s), 3.72 (3H, s), 4.54 (1H, t), 4.63 (1H, d), 7.36 (1H, d), 7.51 (1H, d), 7.82 (1H, d), 8.16 (1H, s), 8.30 (1H, d), 8.41 (1H, s), 8.81 (1H, d), 12.84 (1H, s).

Step 18. Preparation of 5-(5-(2-(1-(2-amino-6-bromo-1H-benzo[d]imidazol-1-yl)-3-azabicyclo[3.2.1]octan-3-yl)ethoxy)-1-methyl-1H-pyrazol-4-yl)-1-methyl-6-oxo-1,6-dihydropyridine-3-carboxylic acid (isomer 2, M4-15B). To a stirred solution of methyl 5-(5-(2-(1-(2-amino-6-bromo-1H-benzo[d]imidazol-1-yl)-3-azabicyclo[3.2.1]octan-3-yl)ethoxy)-1-methyl-1H-pyrazol-4-yl)-1-methyl-6-oxo-1,6-dihydropyridine-3-carboxylate (isomer 2, M4-14B, 4.2 g, 6.88 mmol, 1 equiv) in tetrahydrofuran (80 mL) was added LiOH (0.33 g, 13.76 mmol, 2 equiv) in H₂O (20 mL). The resulting mixture was stirred for 2 h at room temperature. The reaction was concentrated under reduced pressure and the residue was further purified by Prep-HPLC by reverse flash chromatography with C18 silica gel column eluted with water and acetonitrile. Fractions containing the desired compound were evaporated to dryness to give 3.8 g of 5-(5-(2-(1-(2-amino-6-bromo-1H-benzo[d]imidazol-1-yl)-3-azabicyclo[3.2.1]octan-3-yl)ethoxy)-1-methyl-1H-pyrazol-4-yl)-1-methyl-6-oxo-1,6-dihydropyridine-3-carboxylic acid (isomer 2, M4-15B) as a white solid (92%). LCMS: m/z (ESI), [M+H]⁺=598.05.

Step 19. Preparation of 5-bromo-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (isomer 2, M4B). A solution of 5-(5-{2-[1-(2-amino-6-bromo-1,3-benzodiazol-1-yl)-3-azabicyclo[3.2.1]octan-3-yl]ethoxy}-1-methylpyrazol-4-yl)-1-methyl-6-oxopyridine-3-carboxylic acid (M4-15B, 3.8 g, 6.37 mmol, 1 equiv), N,N,N,N-tetramethyl-O-(7-azabenzotriazol-1-yl)uronium hexafluorophospate (3.63 g, 9.56 mmol, 1.5 equiv), and N,N-diisopropylethylamine (2.47 g, 19.11 mmol, 3 equiv) in 1,4-dioxane (80 mL) was stirred for 2 h at room temperature under nitrogen atmosphere. The reaction was quenched with water (300 mL) and the mixture was extracted with ethyl acetate (3×200 mL). The combined organic layers were washed with brine (3×300 mL), dried over anhydrous Na₂SO₄, and concentrated under reduced pressure. The residue was purified on a silica gel column eluted with dichloromethane/methanol (20/1) to give 3.5 g of 5-bromo-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (isomer 2, M4B) as a brown solid (94%). LCMS: m/z (ESI), [M+H]⁺=580.00. SFC-HPLC, Rt=3.186, ¹H NMR (DMSO-d₆, 400 MHz) δ 1.55-1.76 (2H, m), 2.01 (1H, d), 2.39-2.47 (2H, m), 2.51-2.57 (1H, m), 2.61-2.65 (1H, m), 2.70-2.77 (2H, m), 3.09-3.18 (2H, m), 3.19-3.29 (1H, m), 3.63 (3H, s), 3.72 (3H, s), 4.54 (1H, t), 4.63 (1H, d), 7.36 (11H, dd), 7.51 (11H, d), 7.82 (1H, d), 8.16 (1H, s), 8.30 (1H, d), 8.41 (1H, s), 8.81 (1H, d), 12.84 (1H, s)

### Intermediate M5

### 5-Bromo-15,21-dimethyl-23-oxa-2,9,11,15,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,17(34),18(22),19-octaene-12,16-dione

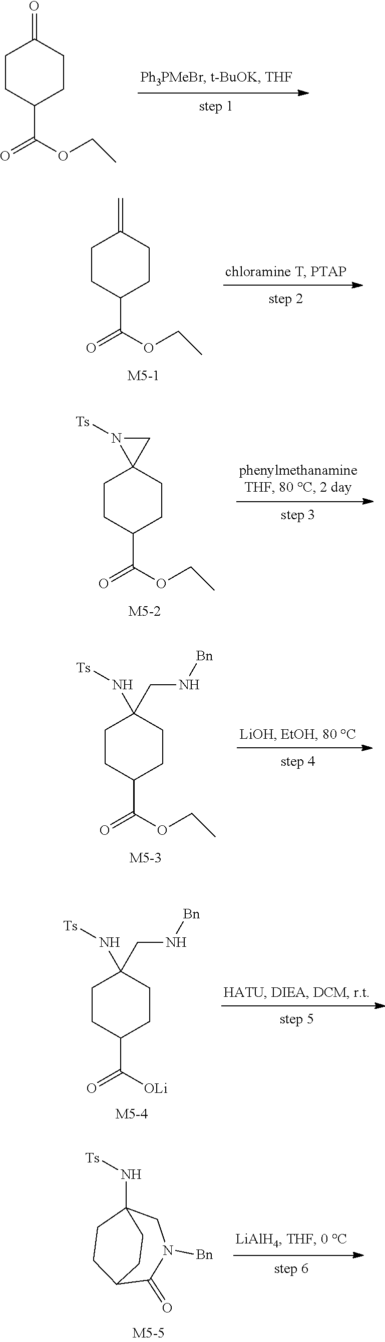

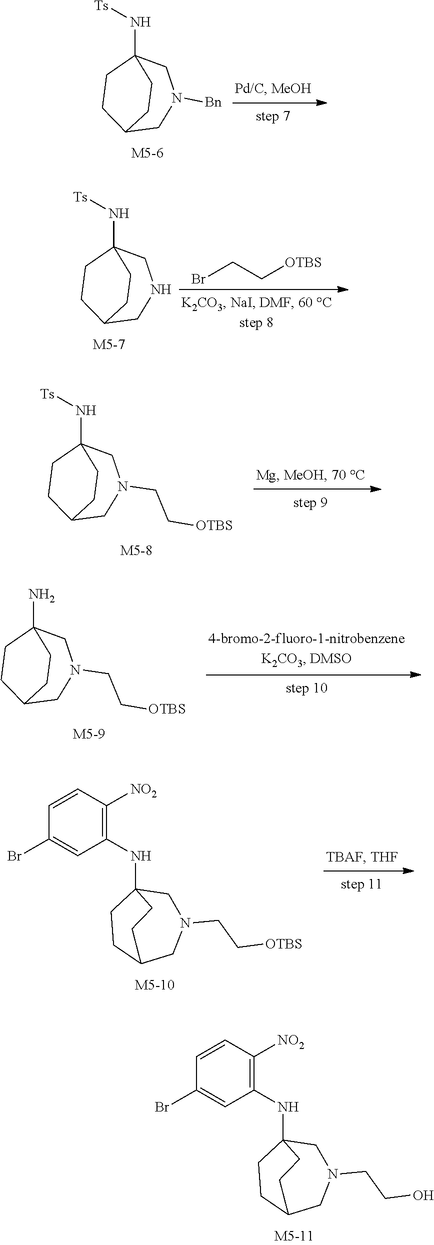

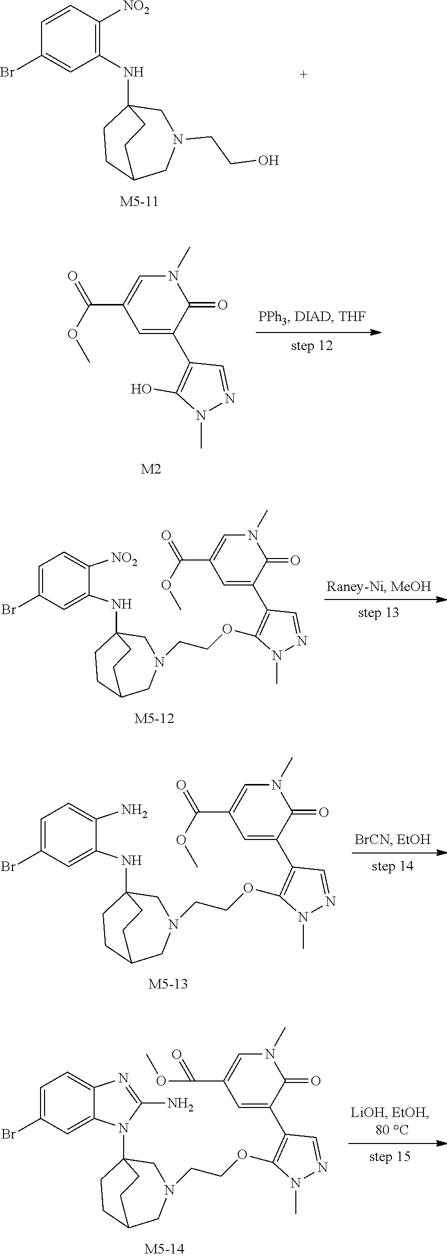 [SMILES: O=[N+]([O-])c1ccc(Br)cc1NC12CCC(CC1)CN(CCO)C2 ; COC(=O)c1cc(-c2cnn(C)c2O)c(=O)n(C)c1 ; "M2" ; COC(=O)c1cc(-c2cnn(C)c2OCCN2CC3CCC(Nc4cc(Br)ccc4[N+](=O)[O-])(CC3)C2)c(=O)n(C)c1 ; COC(=O)c1cc(-c2cnn(C)c2OCCN2CC3CCC(Nc4cc(Br)ccc4N)(CC3)C2)c(=O)n(C)c1 ; COC(=O)c1cc(-c2cnn(C)c2OCCN2CC3CCC(n4c(N)nc5ccc(Br)cc54)(CC3)C2)c(=O)n(C)c1]

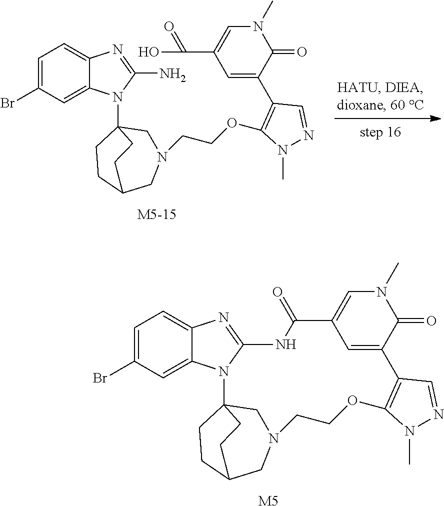

Step 1. Preparation of ethyl 4-methylidenecyclohexane-1-carboxylate (M5-1). To a mixture of methyltriphenylphosphaniumbromide (314.81 g, 881.27 mmol, 1.5 equiv) and potassium tert-butoxide (131.85 g, 1175.03 mmol, 2 equiv) in tetrahydrofuran (500 mL) was added ethyl 4-oxocyclohexane-1-carboxylate (100 g, 587.51 mmol, 1 equiv) at 0 °C. The mixture was stirred for 2 h at room temperature under nitrogen atmosphere. The reaction was quenched with water (300 mL) and the mixture was extracted with ethyl acetate (3×200 mL). The combined organic layers were washed with brine (3×300 mL), dried over anhydrous Na₂SO₄, and concentrated under reduced pressure. The residue was purified by silica gel chromatography eluted with petroleum ether/ethyl acetate (5/1) to give 80 g of ethyl 4-methylidenecyclohexane-1-carboxylate (M5-1) as a yellow oil (85%). ¹H NMR (DMSO-d₆, 400 MHz) δ 1.18 (3H, t), 1.30-1.50 (2H, m), 1.80-1.95 (2H, m), 1.96-2.15 (2H, m), 2.15-2.35 (2H, m), 2.42-2.49 (1H, m), 4.04-4.08 (2H, m), 4.64 (2H, t).

Step 2. Preparation of ethyl 1-(4-methylbenzenesulfonyl)-1-azaspiro[2.5]octane-6-carboxylate (M5-2). To a mixture of ethyl 4-methylidenecyclohexane-1-carboxylate (80 g, 475.52 mmol, 1 equiv) and Chloramine-T (216.50 g, 951.04 mmol, 2 equiv) in acetonitrile (800 mL) was added N,N,N-trimethylanilinium dibromane bromide (17.88 g, 47.55 mmol, 0.1 equiv) at 0 °C. The mixture was stirred overnight at room temperature under nitrogen atmosphere. The resulting mixture was concentrated under reduced pressure. The residue was purified by silica gel chromatography eluted with petroleum ether/ethyl acetate (5/1) to give 60 g of ethyl 1-(4-methylbenzenesulfonyl)-1-azaspiro[2.5]octane-6-carboxylate (M5-2) as a white solid (33%). LCMS: m/z (ESI), [M+H]⁺=338.05. ¹H NMR (DMSO-d₆, 400 MHz) δ 1.18 (3H, t), 1.54-1.58 (1H, m), 1.58-1.72 (1H, m), 1.72-1.93 (4H, m), 2.40 (3H, s), 2.50-2.53 (2H, m), 4.05-4.09 (2H, m), 7.43 (2H, d), 7.73-7.79 (2H, m).

Step 3. Preparation of ethyl 4-[(benzylamino)methyl]-4-(4-methylbenzenesulfonamido)cyclohexane-1-carboxylate (M5-3). A solution of ethyl 1-(4-methylbenzenesulfonyl)-1-azaspiro[2.5]octane-6-carboxylate (60 g, 177.81 mmol, 1 equiv) and benzylamine (28.58 g, 266.72 mmol, 1.5 equiv) in tetrahydrofuran (600 mL) was stirred for 2 days at 80 °C under nitrogen atmosphere. The mixture was cooled to room temperature and concentrated under reduced pressure. The residue was purified by silica gel chromatography eluted with petroleum ether/ethyl acetate (1/1) to give 60 g of ethyl 4-[(benzylamino)methyl]-4-(4-methylbenzenesulfonamido)cyclohexane-1-carboxylate (M5-3) as a white oil (75%). LCMS: m/z (EST), [M+H]⁺=445.20. ¹H NMR (DMSO-d₆, 400 MHz) δ 1.15 (3H, t), 1.31-1.44 (2H, m), 1.48-1.76 (4H, m), 2.00 (1H, m), 2.21-2.30 (1H, m), 2.30 (2H, s), 2.33-2.37 (2H, m), 3.43 (2H, s), 4.02 (2H, m), 7.15-7.25 (3H, m), 7.25-7.36 (4H, m), 7.67-7.75 (2H, m).

Step 4. Preparation of lithium 4-((benzylamino)methyl)-4-((4-methylphenyl)sulfonamido)cyclohexane-1-carboxylate (M5-4). A mixture of ethyl 4-[(benzylamino)methyl]-4-(4-methylbenzenesulfonamido)cyclohexane-1-carboxylate (60 g, 134.95 mmol, 1 equiv) and LiOH (6.46 g, 269.91 mmol, 2 equiv) in ethanol (500 mL) was stirred for 16 h at 80 °C under nitrogen atmosphere. The resulting mixture was concentrated under reduced pressure to give 57 g of lithium 4-((benzylamino)methyl)-4-((4-methylphenyl)sulfonamido)cyclohexane-1-carboxylate (M5-4) as a white solid. This material was used for the next step without further purification. LCMS: m/z (ESI), [M+H]+=417.10.

Step 5. Preparation of N-{3-benzyl-4-oxo-3-azabicyclo[3.2.2]nonan-1-yl}-4-methylbenzenesulfonamide (M5-5). A mixture of N,N,N,N-tetramethyl-O-(7-azabenzotriazol-1-yl)uronium hexafluorophospate (77.03 g, 202.60 mmol, 1.5 equiv), lithium 4-((benzylamino)methyl)-4-((4-methylphenyl)sulfonamido)cyclohexane-1-carboxylate (57 g, ~135 mmol, ~1 equiv), and N,N-diisopropylethylamine (52.37 g, 405.21 mmol, 3 equiv) in dichloromethane (150 mL) was stirred overnight at room temperature under nitrogen atmosphere. The resulting mixture was concentrated under reduced pressure and the residue was purified by silica gel column chromatography eluted with petroleum ether/ethyl acetate (1/1) to give 50 g of N-{3-benzyl-4-oxo-3-azabicyclo[3.2.2]nonan-1-yl}-4-methylbenzenesulfonamide (M5-5) as a white solid (93%). LCMS: m/z (ESI), [M+H]⁺=399.15. ¹H NMR (DMSO-d₆, 400 MHz) δ 1.64-1.74 (8H, m), 2.38 (3H, s), 3.18 (2H, s), 4.37 (2H, s), 7.01-7.19 (2H, m), 7.21-7.41 (5H, m), 7.48-7.56 (2H, m), 7.73 (1H, s)

Step 6. Preparation of N-{3-benzyl-3-azabicyclo[3.2.2]nonan-1-yl}-4-methylbenzenesulfonamide methyl (M5-6). To a mixture of lithium aluminum hydride (7.62 g, 200.74 mmol, 2 equiv) in tetrahydrofuran (400 mL) was added N-{3-benzyl-4-oxo-3-azabicyclo[3.2.2]nonan-1-yl}-4-methylbenzenesulfonamide (40 g, 100.37 mmol, 1 equiv) at 0 °C and the mixture was stirred for 2 h under nitrogen atmosphere. The reaction was quenched with water (7 mL) and 15 mL of NaOH (w/w, 30%) at 0 °C. The mixture was dried over anhydrous Na₂SO₄ and concentrated under reduced pressure. The residue was purified on a silica gel column eluted with petroleum ether/ethyl acetate (1/1) to give 20 g of N-{3-benzyl-3-azabicyclo[3.2.2]nonan-1-yl}-4-methylbenzenesulfonamide (M5-6) as a white solid (46%). LCMS: m/z (ESI), [M+H]⁺=385.15. ¹H NMR (DMSO-d₆, 400 MHz) δ 1.39-1.63 (6H, m), 1.71-1.77 (1H, m), 1.90-2.02 (2H, m), 2.36 (3H, s), 2.40 (2H, d), 2.64 (2H, s), 3.44 (2H, s), 7.18-7.31 (3H, m), 7.31-7.40 (4H, m), 7.39 (1H, s), 7.55-7.62 (2H, m).

Step 7. Preparation of N-{3-azabicyclo[3.2.2]nonan-1-yl}-4-methylbenzenesulfonamide (M5-7). A mixture of Pd/C (10%, 500 mg) and N-{3-benzyl-3-azabicyclo[3.2.2]nonan-1-yl}-4-methylbenzenesulfonamide (20 g, 52.01 mmol, 1 equiv) in methanol (200 mL) was stirred for 4 h at room temperature under hydrogen pressure. The resulting mixture was filtered. The filter cake was washed with methanol (3×70 mL) and the solution was concentrated under reduced pressure. The residue was purified on a silica gel column eluted with dichloromethane/methanol (10/1) to give 15 g of N-{3-azabicyclo[3.2.2]nonan-1-yl}-4-methylbenzenesulfonamide (M5-7) as a white solid (93%). LCMS: m/z (ESI), [M+H]⁺=295.00. ¹H NMR (DMSO-d₆, 400 MHz) δ 1.35-1.60 (6H, m), 1.63-1.69 (1H, m), 1.79-1.83 (2H, m), 2.37 (3H, s), 2.59 (2H, d), 2.80 (2H, s), 3.17 (1H, d), 7.29-7.41 (3H, m), 7.51-7.79 (2H, m).

Step 8. Preparation of N-(3-{2-[(tert-butyldimethylsilyl)oxy]ethyl}-3-azabicyclo[3.2.2]nonan-1-yl)-4-methylbenzenesulfonamide (M5-8). A mixture of N-{3-azabicyclo[3.2.2]nonan-1-yl}-4-methylbenzenesulfonamide (15 g, 50.94 mmol, 1 equiv), NaI (7.64 g, 50.94 mmol, 1 equiv), K₂CO₃ (21.12 g, 152.84 mmol, 3 equiv), and (2-bromoethoxy)(tert-butyl)dimethylsilane (14.63 g, 61.13 mmol, 1.2 equiv) in dimethylformamide (150 mL) was stirred for 16 h at 60 °C under nitrogen atmosphere. The mixture was cooled to room temperature. The reaction was quenched with water (800 mL) and the mixture was extracted with ethyl acetate (3×600 mL). The combined organic layers were washed with brine (3×500 mL), dried over anhydrous Na₂SO₄, and concentrated under reduced pressure. The residue was purified on a silica gel column eluted with dichloromethane/methanol (10/1) to give 20 g of N-(3-{2-[(tert-butyldimethylsilyl)oxy]ethyl}-3-azabicyclo[3.2.2]nonan-1-yl)-4-methylbenzenesulfonamide (M5-8) as a yellow solid (78%). LCMS: m/z (ESI), [M+H]⁺=453.30. ¹H NMR (DMSO-d₆, 400 MHz) δ 0.00 (6H, s), 0.83 (9H, s), 1.32-1.42 (2H, m), 1.44-1.54 (4H, m), 1.65-1.71 (1H, m), 1.80-1.90 (2H, m), 2.34 (3H, s), 2.38 (2H, t), 2.44 (2H, d), 2.65 (2H, s), 3.54 (2H, t), 7.32 (2H, d), 7.37 (1H, s), 7.59-7.76 (2H, m).

Step 9. Preparation of 3-{2-[(tert-butyldimethylsilyl)oxy]ethyl}-3-azabicyclo[3.2.2]nonan-1-amine (M5-9). A mixture of N-(3-{2-[(tert-butyldimethylsilyl)oxy]ethyl}-3-azabicyclo[3.2.2]nonan-1-yl)-4-methylbenzenesulfonamide (5 g, 11.04 mmol, 1 equiv) and Mg (5.37 g, 220.88 mmol, 20 equiv) in methanol (200 mL) was stirred overnight at 70 °C under nitrogen atmosphere. The mixture was cooled to room temperature. The reaction was quenched by the addition of saturated NH₄Cl (aq.) solution (300 mL) and the mixture was extracted with ethyl acetate (3×300 mL). The combined organic layers were washed with brine (3×400 mL), dried over anhydrous Na₂SO₄, and concentrated under reduced pressure. The residue was purified on a silica gel column eluted with dichloromethane/methanol (5/1) to give 2.3 g of 3-{2-[(tert-butyldimethylsilyl)oxy]ethyl}-3-azabicyclo[3.2.2]nonan-1-amine (M5-9) as a yellow oil (60%). ¹H NMR (DMSO-d₆, 400 MHz) δ 0.00 (6H, s), 0.82 (9H, s), 1.54-1.64 (4H, m), 1.79-1.83 (1H, m), 1.85-1.91 (2H, m), 2.42-2.52 (3H, m), 2.54 (2H, d), 2.60 (2H, s), 3.63 (2H, t).

Step 10. Preparation of N-(5-bromo-2-nitrophenyl)-3-{2-[(tert-butyldimethylsilyl)oxy]ethyl}-3-azabicyclo[3.2.2]nonan-1-amine (M5-10). A mixture of 3-{2-[(tert-butyldimethylsilyl)oxy]ethyl}-3-azabicyclo[3.2.2]nonan-1-amine (1 g, 3.35 mmol, 1 equiv), 4-bromo-2-fluoro-1-nitrobenzene (1.84 g, 8.37 mmol, 2.5 equiv), and K₂CO₃ (1.39 g, 10.05 mmol, 3 equiv) in dimethylsulfoxide (10 mL) was stirred for 4 h at 120 °C under nitrogen atmosphere. The mixture was cooled to room temperature. The reaction was quenched with water (50 mL) and the mixture was extracted with ethyl acetate (3×50 mL). The combined organic layers were washed with brine (3×50 mL), dried over anhydrous Na₂SO₄, and concentrated under reduced pressure. The residue was purified by silica gel chromatography eluted with petroleum ether/ethyl acetate (5/1) to give 1.5 g of N-(5-bromo-2-nitrophenyl)-3-{2-[(tert-butyldimethylsilyl)oxy]ethyl}-3-azabicyclo[3.2.2]nonan-1-amine (M5-10) as a yellow oil (85%). LCMS: m/z (ESI), [M+H]⁺=498.15.

Step 11. Preparation of 2-{1-[(5-bromo-2-nitrophenyl)amino]-3-azabicyclo[3.2.2]nonan-3-yl}ethanol (M5-11). A solution of N-(5-bromo-2-nitrophenyl)-3-{2-[(tert-butyldimethylsilyl)oxy]ethyl}-3-azabicyclo[3.2.2]nonan-1-amine (1.5 g, 3.00 mmol, 1 equiv) and tetrabutylammonium fluoride (1.57 g, 6.01 mmol, 2 equiv) in tetrahydrofuran (15 mL) was stirred for 2 h at room temperature under nitrogen atmosphere. The reaction was quenched with sat. NaHCO₃ (aq.) solution (100 mL) and the mixture was extracted with dichloromethane (3×100 mL). The combined organic layers were washed with brine (3×100 mL), dried over anhydrous Na₂SO₄, and concentrated under reduced pressure. The residue was purified by silica gel chromatography eluted with dichloromethane/methanol (10/1) to give 1.2 g of 2-{1-[(5-bromo-2-nitrophenyl)amino]-3-azabicyclo[3.2.2]nonan-3-yl}ethanol (M5-11) as yellow oil (93%). LCMS: m/z (ESI), [M+H]⁺=384.00.

Step 12. Preparation of methyl 5-[5-(2-{1-[(5-bromo-2-nitrophenyl)amino]-3-azabicyclo[3.2.2]nonan-3-yl}ethoxy)-1-methylpyrazol-4-yl]-1-methyl-6-oxopyridine-3-carboxylate (M5-12). To a stirred mixture of methyl 5-(5-hydroxy-1-methylpyrazol-4-yl)-1-methyl-6-oxopyridine-3-carboxylate (M2, 0.66 g, 2.49 mmol, 0.8 equiv) and triphenylphosphine (2.46 g, 9.369 mmol, 3 equiv) in tetrahydrofuran (10 mL) were added diisopropylazodicarboxylate (1.89 g, 9.36 mmol, 3 equiv) and methyl 5-(5-hydroxy-1-methylpyrazol-4-yl)-1-methyl-6-oxopyridine-3-carboxylate (0.66 g, 2.49 mmol, 0.8 equiv) at 0 °C. The mixture was stirred for 2 h at 0 °C under nitrogen atmosphere. The reaction was quenched with water (50 mL) and the mixture was extracted with ethyl acetate (3×50 mL). The combined organic layers were washed with brine (3×50 mL), dried over anhydrous Na₂SO₄, and concentrated under reduced pressure. The residue was purified by Prep-TLC and eluted with petroleum ether/ethyl acetate (1/1) to give 1.2 g of methyl 5-[5-(2-{1-[(5-bromo-2-nitrophenyl)amino]-3-azabicyclo[3.2.2]nonan-3-yl}ethoxy)-1-methylpyrazol-4-yl]-1-methyl-6-oxopyridine-3-carboxylate (M5-12) as a yellow oil (57%). LCMS: m/z (ESI), [M+H]⁺=629.25. ¹H NMR (DMSO-d₆, 400 MHz) δ 1.58-1.76 (9H, m), 2.56-2.79 (2H, m), 2.90 (4H, q), 3.56 (3H, s), 3.72 (6H, d), 4.13 (2H, t), 6.70-6.85 (1H, m), 7.01 (1H, d), 7.95-8.12 (4H, m), 8.28 (1H, d).

Step 13. Preparation of methyl 5-[5-(2-{1-[(2-amino-5-bromophenyl)amino]-3-azabicyclo[3.2.2]nonan-3-yl}ethoxy)-1-methylpyrazol-4-yl]-1-methyl-6-oxopyridine-3-carboxylate (M5-13). To a stirred mixture of Raney Nickel (0.90 g) and methyl 5-[5-(2-{1-[(5-bromo-2-nitrophenyl)amino]-3-azabicyclo[3.2.2]nonan-3-yl}ethoxy)-1-methylpyrazol-4-yl]-1-methyl-6-oxopyridine-3-carboxylate (1.1 g, 1.74 mmol, 1 equiv) in methanol (8 mL) was added NH₂NH₂·H₂O (47 mg, 0.95 mmol, 2 equiv) at 0 °C under nitrogen atmosphere. The mixture was stirred for 1 h at 0 °C. The resulting mixture was filtered. The filter cake was washed with methanol (2×20 mL) and the solution was concentrated under reduced pressure. The residue was purified by Prep-TLC and eluted with dichloromethane/methanol (10/1) to give 910 mg of methyl 5-[5-(2-{1-[(2-amino-5-bromophenyl)amino]-3-azabicyclo[3.2.2]nonan-3-yl}ethoxy)-1-methylpyrazol-4-yl]-1-methyl-6-oxopyridine-3-carboxylate (M5-13) as a light yellow solid (76%). LCMS: m/z (ESI), [M+H]⁺=599.20.

Step 14. Preparation of methyl 5-(5-{2-[1-(2-amino-6-bromo-1,3-benzodiazol-1-yl)-3-azabicyclo[3.2.2]nonan-3-yl]ethoxy}-1-methylpyrazol-4-yl)-1-methyl-6-oxopyridine-3-carboxylate (M5-14). A mixture of methyl 5-[5-(2-{1-[(2-amino-5-bromophenyl)amino]-3-azabicyclo[3.2.2]nonan-3-yl}ethoxy)-1-methylpyrazol-4-yl]-1-methyl-6-oxopyridine-3-carboxylate (900 mg, 1.50 mmol, 1 equiv) and BrCN (174 mg, 1.65 mmol, 1.1 equiv) in ethyl alcohol (10 mL) was stirred for 2 h at room temperature under nitrogen atmosphere. The mixture was concentrated under vacuum. The residue was purified by Prep-TLC and eluted with dichloromethane/methanol (12/1) to give 640 mg of methyl 5-(5-{2-[1-(2-amino-6-bromo-1,3-benzodiazol-1-yl)-3-azabicyclo[3.2.2]nonan-3-yl]ethoxy}-1-methylpyrazol-4-yl)-1-methyl-6-oxopyridine-3-carboxylate (M5-14) as a light yellow solid (68%). LCMS: m/z (EST), [M+H]⁺=624.10. ¹H NMR (DMSO-d₆, 400 MHz) δ 1.24 (1H, s), 1.78-1.95 (8H, m), 2.60-2.74 (2H, m), 2.85-2.95 (4H, m), 3.57 (3H, s), 3.71 (6H, d), 4.08-4.17 (2H, m), 5.95 (2H, s), 7.04 (2H, s), 7.47 (1H, s), 7.96 (1H, s), 8.12 (1H,), 8.39 (1H, d).

Step 15. Preparation of 5-(5-{2-[1-(2-amino-6-bromo-1,3-benzodiazol-1-yl)-3-azabicyclo[3.2.2]nonan-3-yl]ethoxy}-1-methylpyrazol-4-yl)-1-methyl-6-oxopyridine-3-carboxylic acid (M5-15). A mixture of methyl 5-(5-{2-[1-(2-amino-6-bromo-1,3-benzodiazol-1-yl)-3-azabicyclo[3.2.2]nonan-3-yl]ethoxy}-1-methylpyrazol-4-yl)-1-methyl-6-oxopyridine-3-carboxylate (620 mg, 0.99 mmol, 1 equiv) and LiOH (28 mg, 1.19 mmol, 1.2 equiv) in tetrahydrofuran/H₂O (4 mL/1 mL) was stirred for 2 h at 80 °C under nitrogen atmosphere. The mixture was cooled to room temperature and concentrated under vacuum to give 550 mg of 5-(5-{2-[1-(2-amino-6-bromo-1,3-benzodiazol-1-yl)-3-azabicyclo[3.2.2]nonan-3-yl]ethoxy}-1-methylpyrazol-4-yl)-1-methyl-6-oxopyridine-3-carboxylic acid (M5-15) as a white solid (90%). LCMS: m/z (ESI), [M+H]⁺=610.10.

Step 16. Preparation of 5-bromo-15,21-dimethyl-23-oxa-2,9,11,15,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,17(34),18(22),19-octaene-12,16-dione (M5). A mixture of 5-(5-{2-[1-(2-amino-6-bromo-1,3-benzodiazol-1-yl)-3-azabicyclo[3.2.2]nonan-3-yl]ethoxy}-1-methylpyrazol-4-yl)-1-methyl-6-oxopyridine-3-carboxylic acid (550 mg, 0.90 mmol, 1 equiv), N,N,N,N-tetramethyl-O-(7-azabenzotriazol-1-yl)uronium hexafluorophospate (1027 mg, 2.70 mmol, 3 equiv) and N,N-diisopropylethylamine (174 mg, 1.35 mmol, 1.5 equiv) in dioxane (6 mL) was stirred for 2 h at 60 °C under nitrogen atmosphere. The mixture was cooled to room temperature and concentrated under vacuum. The residue was purified by Prep-HPLC with XBridge Prep OBD C18 Column using in water containing 10 mmol/L NH₄HCO₃ and acetonitrile as mobile phase. Fractions containing the desired compound were evaporated to dryness to give 500 mg of 5-bromo-15,21-dimethyl-23-oxa-2,9,11,15,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,17(34),18(22),19-octaene-12,16-dione (M5) as a yellow solid (88%). LCMS: m/z (ESI), [M+H]⁺=592.15.

### SYNTHESIS OF EXAMPLES

### Example A1

### 5,26-Dimethyl-10-(2,2,2-trifluoroethyl)-7-oxa-4,5,10,13,20,22,26-heptaazapentacyclo[22.3.1.0\hat{ }{2,6}.0\hat{ }{13,21}.0\hat{ }{14,19}]octacosa-1(28),2(6),3,14,16,18,20,24-octaene-23,27-dione

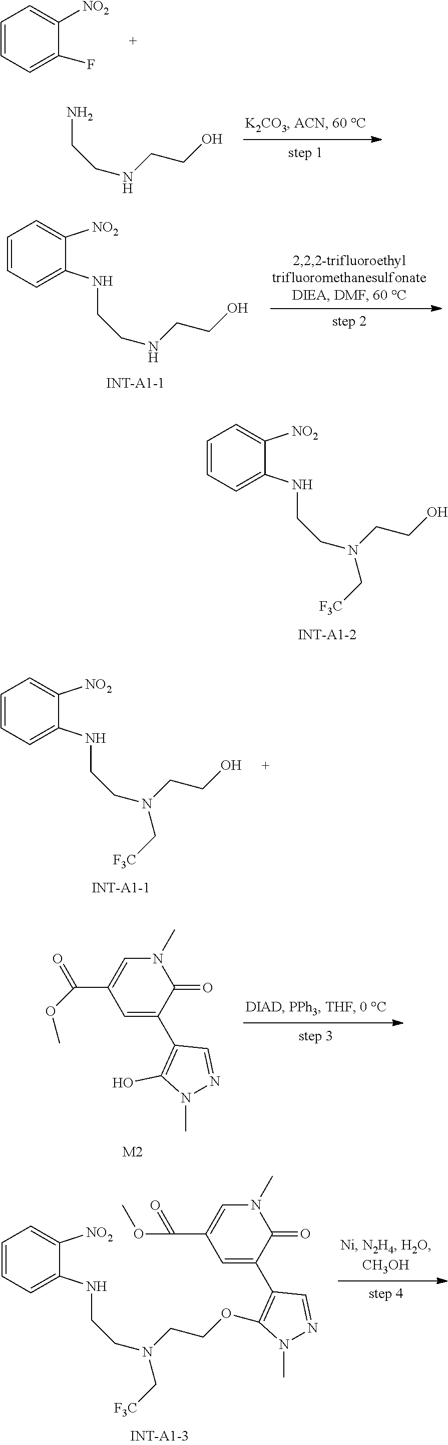

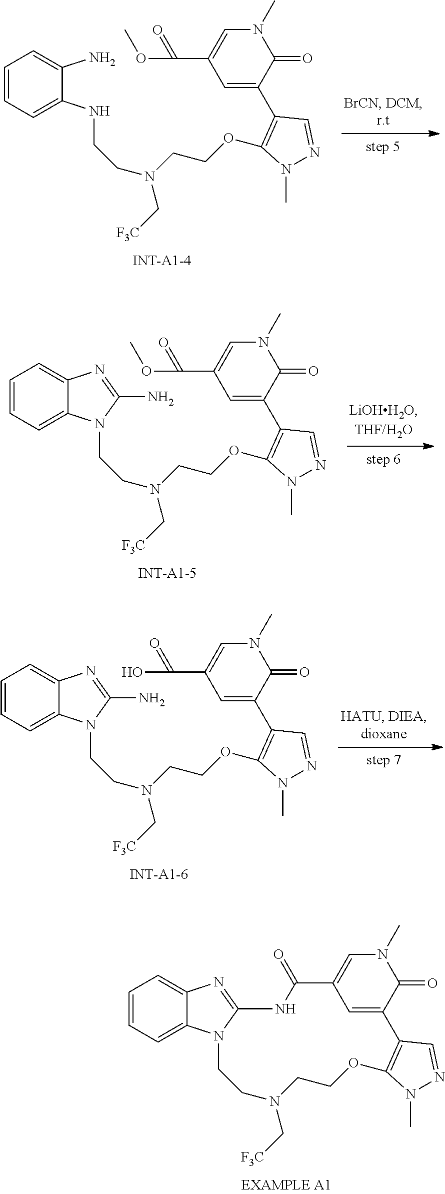 [SMILES: COC(=O)c1cc(-c2cnn(C)c2OCCN(CCNc2ccccc2N)CC(F)(F)F)c(=O)n(C)c1 ; COC(=O)c1cc(-c2cnn(C)c2OCCN(CCn2c(N)nc3ccccc32)CC(F)(F)F)c(=O)n(C)c1 ; Cn1ncc(-c2cc(C(=O)O)cn(C)c2=O)c1OCCN(CCn1c(N)nc2ccccc21)CC(F)(F)F ; Cn1ncc2c1OCCN(CC(F)(F)F)CCn1c(nc3ccccc31)NC(=O)c1cc-2c(=O)n(C)c1]

Step 1. Preparation of 2-((2-((2-nitrophenyl)amino)ethyl)amino)ethan-1-ol (INT-A 1-1). A mixture of O-fluoronitrobenzene (10 g, 70.871 mmol, 1 equiv), K₂CO₃ (19.59 g, 141.742 mmol, 2 equiv) and aminoethylethanolamine (14.76 g, 141.742 mmol, 2 equiv) in ACN (300 mL) was stirred for 2 h at 60 °C under nitrogen atmosphere. The mixture was cooled to room temperature. The reaction was quenched with water (800 mL) and the mixture was extracted with ethyl acetate (3×600 mL). The combined organic layers were washed with brine (3×500 mL), dried over anhydrous Na₂SO₄, and concentrated under reduced pressure. The residue was purified by silica gel chromatography and eluted with PE/EA (1:1) to afford 8.5 g of 2-({2-[(2-nitrophenyl)amino]ethyl}amino)ethanol (INT-A1-1) as a yellow solid (53%). ¹H NMR (DMSO-d₆, 400 MHz) δ 1.99 (1H, s), 2.62 (2H, t), 2.84 (2H, t), 3.36-3.40 (2H, m), 3.44-3.49 (2H, m), 4.50 (1H, t), 6.65-3.71 (1H, m), 7.04-7.08 (1H, m), 7.54 (1H, m), 8.34-8.38 (1H, t), 8.04-8.08 (1H, m).

Step 2. Preparation of 2-({2-[(2-nitrophenyl)amino]ethyl}(2,2,2-trifluoroethyl)amino)ethanol (INT-A1-2). A mixture of 2-({2-[(2-nitrophenyl)amino]ethyl}amino)ethanol (INT-A1-1, 2 g, 8.879 mmol, 1 equiv), N,N-diisopropylethylamine (2.30 g, 17.758 mmol, 2 equiv), and 2,2,2-trifluoroethyl trifluoromethanesulfonate (2.47 g, 10.655 mmol, 1.2 equiv) in N,N-dimethylformamide (20 mL) was stirred for 2 h at 60 °C under nitrogen atmosphere. The mixture was cooled to room temperature. The reaction was quenched with water (200 mL) and the mixture was extracted with dichloromethane (3×200 mL). The combined organic layers were washed with brine (3×200 mL), dried over anhydrous Na₂SO₄, and concentrated under reduced pressure. The residue was purified by silica gel chromatography eluted with petroleum ether/ethyl acetate (1/1) to afford 2.19 g of 2-({2-[(2-nitrophenyl)amino]ethyl}(2,2,2-trifluoroethyl)amino)ethanol (INT-A1-2) as a yellow solid (80%). LCMS: m/z (ESI), [M+H]⁺=308.10.

Step 3. Preparation of methyl 1-methyl-5-{1-methyl-5-[2-({2-[(2-nitrophenyl)amino]ethyl}(2,2,2-trifluoroethyl)amino)ethoxy]pyrazol-4-yl}-6-oxopyridine-3-carboxylate (INT-A1-3). To a stirred mixture of methyl 5-(5-hydroxy-1-methylpyrazol-4-yl)-1-methyl-6-oxopyridine-3-carboxylate (M2, 0.56 g, 2.14 mmol, 1 equiv) and triphenylphosphine (1.69 g, 6.44 mmol, 3 equiv) in tetrahydrofuran (20 mL) were added diisopropyl azodicarboxylate (1.30 g, 6.44 mmol, 3 equiv) and 2-({2-[(2-nitrophenyl)amino]ethyl}(2,2,2-trifluoroethyl)amino)ethanol (0.66 g, 2.15 mmol, 1 equiv) at 0 °C under nitrogen atmosphere. The mixture was stirred for 2 h at 0 °C under nitrogen atmosphere. The reaction was quenched with water (150 mL) and the mixture was extracted with dichloromethane (3×150 mL). The combined organic layers were washed with brine (3×150 mL), dried over anhydrous Na₂SO₄, and concentrated under reduced pressure. The residue was purified on a Prep-TLC eluted with petroleum ether/ethyl acetate (1/3) to give 1.15 g of methyl 1-methyl-5-{1-methyl-5-[2-({2-[(2-nitrophenyl)amino]ethyl}(2,2,2-trifluoroethyl)amino)ethoxy]pyrazol-4-yl}-6-oxopyridine-3-carboxylate (INT-A1-3) as an orange oil (96%). LCMS: m/z (ESI), [M+H]⁺=553.25. ¹H NMR (DMSO-d₆, 400 MHz) δ 3.02 (2H, t), 3.12-3.19 (2H, m), 3.38-3.44 (2H, m), 3.50 (1H, s), 3.56 (3H, s), 3.67 (3H, s), 3.76 (3H, s), 4.03 (2H, t), 6.63-6.72 (1H, m), 6.97-7.05 (1H, m), 7.50-7.58 (1H, m), 7.62-7.68 (1H, m), 7.92 (1H, s), 8.01-8.06 (2H, m), 8.20 (1H, t), 8.37 (1H, d)

Step 4. Preparation of methyl 5-{5-[2-({2-[(2-aminophenyl)amino]ethyl}(2,2,2-trifluoroethyl)amino)ethoxy]-1-methylpyrazol-4-yl}-1-methyl-6-oxopyridine-3-carboxylate (INT-A1-4). To a stirred mixture of methyl 1-methyl-5-{1-methyl-5-[2-({2-[(2-nitrophenyl)amino]ethyl}(2,2,2-trifluoroethyl)amino)ethoxy]pyrazol-4-yl}-6-oxopyridine-3-carboxylate (1.1 g, 1.99 mmol, 1 equiv) and Raney Nickel (0.85 g, 9.95 mmol, 5 equiv) in methanol (30 mL) was added hydrazine hydrate (0.15 g, 2.98 mmol, 1.5 equiv) at 0 °C. The mixture was stirred for 30 min at 0 °C under nitrogen atmosphere. The resulting mixture was filtered. The filter cake was washed with methanol (3×20 mL) and the solution was concentrated under reduced pressure to give 1 g of methyl 5-{5-[2-({2-[(2-aminophenyl)amino]ethyl}(2,2,2-trifluoroethyl)amino)ethoxy]-1-methylpyrazol-4-yl}-1-methyl-6-oxopyridine-3-carboxylate (INT-A1-4) as a brown solid (96%). This material was used for the next reaction without further purification. LCMS: m/z (ESI), [M+H]⁺=523.10. ¹H NMR (DMSO-d₆, 400 MHz) δ 2.95 (2H, t), 3.12 (2H, t), 3.17 (2H, d), 3.48 (2H, q), 3.57 (3H, s), 3.68 (3H, s), 3.79 (3H, s), 4.03 (2H, t), 4.36 (2H, s), 6.37-6.46 (2H, m), 7.51-7.59 (2H, m), 7.64-7.67 (1H, m), 7.94 (1H, s), 8.10 (1H, d), 8.45 (1H, d).

Step 5. Preparation of methyl 5-[5-(2-{[2-(2-amino-1,3-benzodiazol-1-yl)ethyl](2,2,2-trifluoroethyl)amino}ethoxy)-1-methylpyrazol-4-yl]-1-methyl-6-oxopyridine-3-carboxylate (INT-A1-5). A mixture of methyl 5-{5-[2-({2-[(2-aminophenyl)amino]ethyl}(2,2,2-trifluoroethyl)amino)ethoxy]-1-methylpyrazol-4-yl}-1-methyl-6-oxopyridine-3-carboxylate (1 g, ~1.9 mmol, ~1 equiv) and BrCN (0.24 g, 2.29 mmol, 1.2 equiv) in CH₂Cl₂ (20 mL) was stirred for 2 h at room temperature under nitrogen atmosphere. The mixture was concentrated under reduced pressure. The residue was purified by Prep-TLC eluted with CH₂Cl₂/methanol (40/1) to give 1 g of methyl 5-[5-(2-{[2-(2-amino-1,3-benzodiazol-1-yl)ethyl](2,2,2-trifluoroethyl)amino}ethoxy)-1-methylpyrazol-4-yl]-1-methyl-6-oxopyridine-3-carboxylate (INT-A01-5) as a brown solid (95%). LCMS: m/z (EST), [M+H]⁺=548.25. ¹H NMR (DMSO-d₆, 400 MHz) δ 3.04 (2H, t), 3.16 (2H, t), 3.50 (2H, q), 3.58 (3H, s), 3.65 (3H, s), 3.77 (3H, s), 3.98 (2H, t), 4.18 (2H, t), 7.07-7.21 (2H, m), 7.32 (1H, d), 7.40 (1H, d), 7.93 (1H, s), 8.04 (1H, d), 8.11 (2H, s), 8.46 (1H, d).

Step 6. Preparation of 5-[5-(2-{[2-(2-amino-1,3-benzodiazol-1-yl)ethyl](2,2,2-trifluoroethyl)amino}ethoxy)-1-methylpyrazol-4-yl]-1-methyl-6-oxopyridine-3-carboxylic acid (INT-A1-6). A mixture of methyl 5-[5-(2-{[2-(2-amino-1,3-benzodiazol-1-yl)ethyl](2,2,2-trifluoroethyl)amino}ethoxy)-1-methylpyrazol-4-yl]-1-methyl-6-oxopyridine-3-carboxylate (200 mg, 0.36 mmol, 1 equiv) and LiOH·H₂O (23 mg, 0.55 mmol, 1.5 equiv) in tetrahydrofuran (4 mL) and H₂O (1 mL) was stirred overnight at room temperature under nitrogen atmosphere. The reaction mixture was concentrated under reduced pressure. The residue was purified by C18 FLASH using water and acetonitrile as mobile phase. Fractions containing the desired compound were evaporated to dryness to give 180 mg 5-[5-(2-{[2-(2-amino-1,3-benzodiazol-1-yl)ethyl](2,2,2-trifluoroethyl)amino}ethoxy)-1-methylpyrazol-4-yl]-1-methyl-6-oxopyridine-3-carboxylic acid (INT-A1-6) as a white solid (92%). LCMS: m/z (ESI), [M+H]⁺=534.10. ¹H NMR (DMSO-d₆, 400 MHz) δ 2.99 (2H, t), 3.28 (2H, t), 3.52 (6H, s), 3.67 (3H, s), 3.99 (2H, t), 4.18 (2H, t), 6.80-7.01 (4H, m), 7.05-7.14 (2H, m), 8.02 (1H, s), 8.09 (1H, d), 8.37 (1H, d)

Step 7. Preparation of 5,26-dimethyl-10-(2,2,2-trifluoroethyl)-7-oxa-4,5,10,13,20,22,26-heptaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (EXAMPLE A1). A mixture of 5-[5-(2-{[2-(2-amino-1,3-benzodiazol-1-yl)ethyl](2,2,2-trifluoroethyl)amino}ethoxy)-1-methylpyrazol-4-yl]-1-methyl-6-oxopyridine-3-carboxylic acid (150 mg, 0.28 mmol, 1 equiv), N,N,N,N-tetramethyl-O-(7-azabenzotriazol-1-yl)uronium hexafluorophospate (160 mg, 0.42 mmol, 1.5 equiv) and N,N-diisopropylethylamine (109 mg, 0.84 mmol, 3 equiv) in 1,4-dioxane (5 mL) was stirred overnight at room temperature under nitrogen atmosphere. The reaction was quenched with water (50 mL) and the mixture was extracted with ethyl acetate (3×50 mL). The combined organic layers were washed with brine (3×150 mL), dried over anhydrous Na₂SO₄, and concentrated under reduced pressure. The residue was purified by prep-HPLC with XBridge Shield RP18 OBD Column using water containing 10 mmol/L NH₄HCO₃ and 0.10% NH₃·H₂O and acetonitrile as mobile phase. Fractions containing the desired compound were evaporated to dryness to give 29.5 mg of 5,26-dimethyl-10-(2,2,2-trifluoroethyl)-7-oxa-4,5,10,13,20,22,26-heptaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (EXAMPLE A1) as a pink solid (20%). LCMS: m/z (ESI), [M+H]⁺=516.20. ¹H NMR (DMSO, 400 MHz) δ 3.10 (2H, t), 3.27 (2H, t), 3.63 (3H, s), 3.59-3.69 (2H, m), 3.71 (3H, s), 4.19 (2H, t), 4.33 (2H, t), 7.18-7.31 (2H, m), 7.49-7.56 (1H, m), 7.70 (1H, d), 8.25 (1H, s), 8.29 (1H, d), 8.89 (1H, d), 12.55 (1H, s). ¹⁹F NMR (DMSO-d₆, 376 MHz) δ 69.23 (s).

### Example A2

### 5-Cyclopropyl-26-methyl-16-(4-methylpiperazin-1-yl)-10-(2,2,2-trifluoroethyl)-7-oxa-4,5,10,13,20,22,26-heptaazapentacyclo[22.3.1.0\hat{ }{2,6}.0\hat{ }{13,21}.0\hat{ }{14,19}]octacosa-1(28),2(6),3,14,16,18,20,24-octaene-23,27-dione

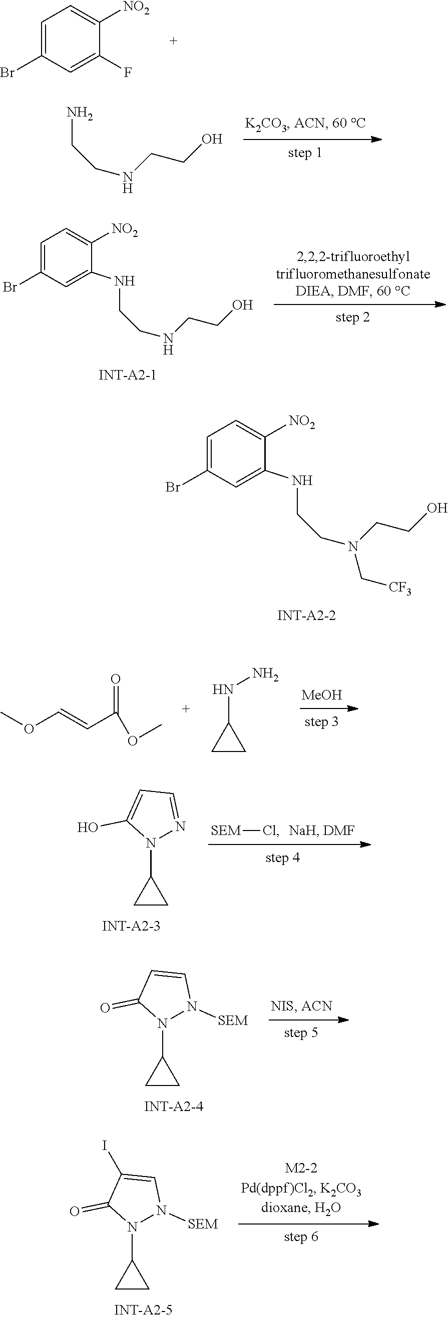

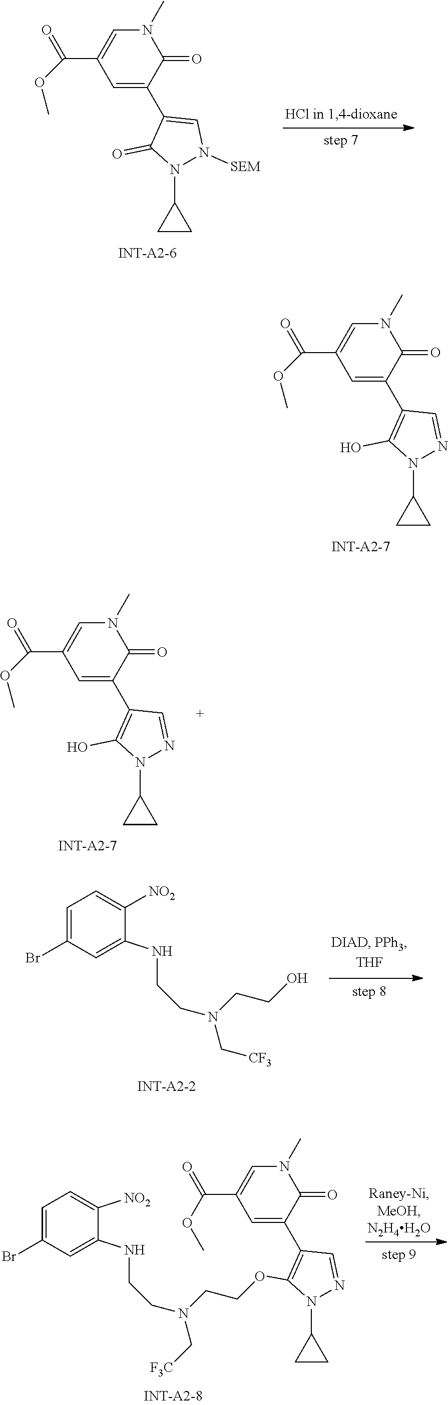 [SMILES: COC(=O)c1cc(-c2cn(COCC[Si](C)(C)C)n(C3CC3)c2=O)c(=O)n(C)c1 ; COC(=O)c1cc(-c2cnn(C3CC3)c2O)c(=O)n(C)c1 ; COC(=O)c1cc(-c2cnn(C3CC3)c2O)c(=O)n(C)c1 ; O=[N+]([O-])c1ccc(Br)cc1NCCN(CCO)CC(F)(F)F ; COC(=O)c1cc(-c2cnn(C3CC3)c2OCCN(CCNc2cc(Br)ccc2[N+](=O)[O-])CC(F)(F)F)c(=O)n(C)c1]

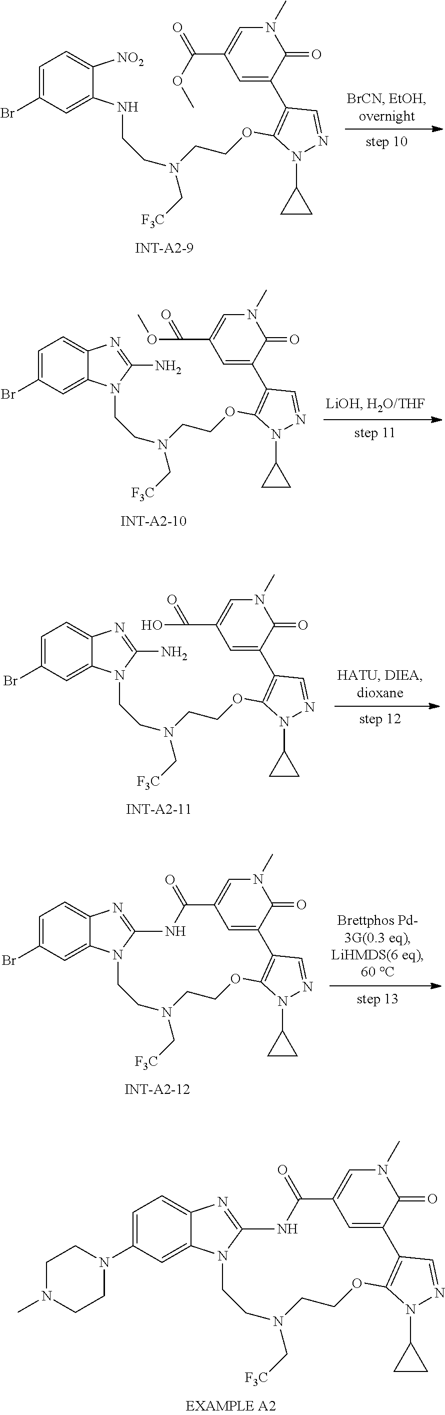 [SMILES: COC(=O)c1cc(-c2cnn(C3CC3)c2OCCN(CCNc2cc(Br)ccc2[N+](=O)[O-])CC(F)(F)F)c(=O)n(C)c1 ; COC(=O)c1cc(-c2cnn(C3CC3)c2OCCN(CCn2c(N)nc3ccc(Br)cc32)CC(F)(F)F)c(=O)n(C)c1 ; Cn1cc(C(=O)O)cc(-c2cnn(C3CC3)c2OCCN(CCn2c(N)nc3ccc(Br)cc32)CC(F)(F)F)c1=O ; Cn1cc2cc(c1=O)-c1cnn(C3CC3)c1OCCN(CC(F)(F)F)CCn1c(nc3ccc(Br)cc31)NC2=O ; CN1CCN(c2ccc3nc4n(c3c2)CCN(CC(F)(F)F)CCOc2c(cnn2C2CC2)-c2cc(cn(C)c2=O)C(=O)N4)CC1]

Step 1. Preparation of 2-({2-[(5-bromo-2-nitrophenyl)amino]ethyl}amino)ethanol (INT-A2-1). A mixture of 4-bromo-2-fluoro-1-nitrobenzene (2 g, 9.09 mmol, 1 equiv), aminoethylethanolamine (1.42 g, 13.63 mmol, 1.5 equiv), and K₂CO₃ (3 g, 27.27 mmol, 3 equiv) in acetonitrile (20 mL) was stirred for 2 h at 60 °C under nitrogen atmosphere. The mixture was cooled to room temperature. The reaction was quenched with water (100 mL) and the mixture was extracted with ethyl acetate (3×100 mL). The combined organic layers were washed with brine (3×100 mL), dried over anhydrous Na₂SO₄, and concentrated under reduced pressure. The residue was purified by Prep-TLC eluted with dichloromethane/methanol (10/1) to give 2.1 g of 2-({2-[(5-bromo-2-nitrophenyl)amino]ethyl}amino)ethanol (INT-A2-1) as a yellow solid (72%). LCMS: m/z (ESI), [M+H]⁺=306.00. ¹H NMR (DMSO-d₆, 400 MHz) δ 2.64 (2H, t), 2.83 (2H, t), 3.18 (1H, d), 3.37-3.44 (2H, m), 3.48 (2H, q), 4.53 (1H, t), 6.83 (1H, d), 7.28 (1H, d), 7.99 (1H, d), 8.42 (1H, t).

Step 2. Preparation of 2-({2-[(5-bromo-2-nitrophenyl)amino]ethyl}(2,2,2-trifluoroethyl)amino)ethanol (INT-A2-2). To a stirred mixture of 2-({2-[(5-bromo-2-nitrophenyl)amino]ethyl}amino)ethanol (INT-A2-1, 10 g, 32.87 mmol, 1 equiv) and N,N-diisopropylethylamine (12 g, 98.63 mmol, 3 equiv) in dimethylformamide (100 mL) was added 2,2,2-trifluoroethyl trifluoromethanesulfonate (11 g, 49.31 mmol, 1.5 equiv). The mixture was stirred for 2 h at 60 °C under nitrogen atmosphere. The mixture was cooled to room temperature. The reaction was quenched with water (100 mL) and the mixture was extracted with ethyl acetate (3×100 mL). The combined organic layers were washed with brine (3×100 mL), dried over anhydrous Na₂SO₄, and concentrated under reduced pressure. The residue was purified by prep-TLC eluting with petroleum ether/ethyl acetate (5/1) to give 3 g of 2-({2-[(5-bromo-2-nitrophenyl)amino]ethyl}(2,2,2-trifluoroethyl)amino)ethanol (INT-A2-2) as a yellow oil (21%). LCMS: m/z (ESI), [M+H]⁺=385.90 ¹H NMR (DMSO-d₆, 400 MHz) δ 2.76 (2H, t), 2.95 (2H, t), 3.40 (4H, d), 3.57-3.45 (2H, m), 4.55 (1H, t), 6.84 (1H, d), 7.27 (1H, d), 8.00 (1H, d), 8.30 (1H, t).

Step 3. Preparation of 2-cyclopropylpyrazol-3-ol (INT-A2-3). A mixture of methyl (2E)-3-methoxyprop-2-enoate (1 g, 8.61 mmol, 1 equiv) and cyclopropylhydrazine (621 mg, 8.61 mmol, 1 equiv) in methanol (4 mL) was stirred for 16 h at 80 °C under air atmosphere. After being cooled to room temperature, the reaction was quenched with sat. NH₄Cl solution (300 mL) and the mixture was extracted with dichloromethane (3×200 mL). The combined organic layers were washed with brine (3×300 mL), dried over anhydrous Na₂SO₄, and concentrated under reduced pressure. The residue was purified by reverse flash chromatography eluted with 0% to 15% acetonitrile in water to give 200 mg of 2-cyclopropylpyrazol-3-ol (INT-A2-3) as a yellow oil (18%). LCMS: m/z (ESI), [M+H]=125.15. ¹H NMR (DMSO-d₆, 400 MHz) δ 1.08-0.93 (4H, m), 3.44-3.31 (1H, m), 5.64 (1H, d), 7.62 (1H, d).

Step 4. Preparation of 2-cyclopropyl-1-{[2-(trimethylsilyl)ethoxy]methyl}pyrazol-3-one (INT-A2-4). To a stirred mixture of 2-cyclopropylpyrazol-3-ol (1.1 g, 8.86 mmol, 1 equiv) and 2-(trimethylsily)ethoxymethyl chloride (2.66 g, 15.95 mmol, 1.8 equiv) in N,N-dimethylformamide (10 mL) was added sodium hydride (0.85 g, 17.72 mmol, 2 equiv, 60%) in portions at room temperature under nitrogen atmosphere. The mixture was stirred for 2 h. The reaction was quenched with sat. NH₄Cl solution (300 mL) and the mixture was extracted with dichloromethane (3×200 mL). The combined organic layers were washed with brine (3×300 mL), dried over anhydrous Na₂SO₄, and concentrated under reduced pressure. The residue was purified on a silica gel column eluted with dichloromethane/methanol (10/1). The product was further purified by reverse flash chromatography eluted with 0% to 100% acetonitrile in water to give 330 mg of 2-cyclopropyl-1-{[2-(trimethylsilyl)ethoxy]methyl}pyrazol-3-one (INT-A2-4) as a brown solid (15%). LCMS: m/z (ESI), [M+H]⁺=255.20

Step 5. Preparation of 2-cyclopropyl-4-iodo-1-{[2-(trimethylsilyl)ethoxy]methyl}pyrazol-3-one (INT-A2-5). A mixture of 2-cyclopropyl-1-{[2-(trimethylsilyl)ethoxy]methyl}pyrazol-3-one (4.5 g, 17.69 mmol, 1 equiv) and NIS (5.97 g, 26.53 mmol, 1.5 equiv) in acetonitrile (50 mL) was stirred for 2 h at 0 °C under nitrogen atmosphere. The reaction was quenched by the addition of sat. sodium hyposulfite (aq.) (100 mL) and the mixture was extracted with CH₂Cl₂ (3×50 mL). The combined organic layers were washed with brine (3×100 mL), dried over anhydrous Na₂SO₄, and concentrated under reduced pressure to give 5.5 g of 2-cyclopropyl-4-iodo-1-{[2-(trimethylsilyl)ethoxy]methyl}pyrazol-3-one (INT-A2-5) as a yellow solid (82%). LCMS: m/z (ESI), [M+H]⁺=380.95

Step 6. Preparation of methyl 5-(2-cyclopropyl-3-oxo-1-{[2-(trimethylsilyl)ethoxy]methyl}pyrazol-4-yl)-1-methyl-6-oxopyridine-3-carboxylate (INT-A2-6). A mixture of freshly prepared 5-(methoxycarbonyl)-1-methyl-2-oxopyridin-3-ylboronic acid (M2-2, 800 mg, 3.8 mmol, 1 equiv), K₂CO₃ (1.31 g, 9.48 mmol, 2.5 equiv), Pd(dppf)Cl₂ (554 mg, 0.76 mmol, 0.2 equiv) and 2-cyclopropyl-4-iodo-1-{[2-(trimethylsilyl)ethoxy]methyl}pyrazol-3-one (1.73 g, 4.55 mmol, 1.2 equiv) in 1,4-dioxane (8 mL) and H₂O (2 mL) was stirred for 2 h at 80 °C under nitrogen atmosphere. After being cooled to room temperature, the reaction was quenched with water (300 mL) and the mixture was extracted with dichloromethane (3×200 mL). The combined organic layers were washed with brine (3×300 mL), dried over anhydrous Na₂SO₄, and concentrated under reduced pressure. The residue was purified on a silica gel column eluted with petroleum ether/ethyl acetate (1/1) to give 1 g of methyl 5-(2-cyclopropyl-3-oxo-1-{[2-(trimethylsilyl)ethoxy]methyl}pyrazol-4-yl)-1-methyl-6-oxopyridine-3-carboxylate (INT-A22-4) as a brown solid (92%). LCMS: m/z (ESI), [M+H]⁺=420.05.

Step 7. Preparation of methyl 5-(1-cyclopropyl-5-hydroxypyrazol-4-yl)-1-methyl-6-oxopyridine-3-carboxylate (INT-A2-7). A solution of methyl 5-(2-cyclopropyl-3-oxo-1-{[2-(trimethylsilyl)ethoxy]methyl}pyrazol-4-yl)-1-methyl-6-oxopyridine-3-carboxylate (750 mg, 1.79 mmol, 1 equiv) in HCl in 1,4-dioxane (10 mL) was stirred for 5 h at 60 °C under nitrogen atmosphere. The resulting mixture was cooled to room temperature and concentrated under reduced pressure. The residue was dissolved in methanol (10 mL), neutralized to pH 7 with K₂CO₃. The mixture was filtered. The filter cake was washed with methanol (2×5 mL). The solution was concentrated under reduced pressure to give 500 mg of methyl 5-(1-cyclopropyl-5-hydroxypyrazol-4-yl)-1-methyl-6-oxopyridine-3-carboxylate (INT-A2-7) as a brown solid (96%). This material was used for the next step without further purification. LCMS: m/z (ESI), [M+H]⁺=290.05

Step 8. Preparation of methyl 5-{5-[2-({2-[(5-bromo-2-nitrophenyl)amino]ethyl}(2,2,2-trifluoroethyl)amino)ethoxy]-1-cyclopropylpyrazol-4-yl}-1-methyl-6-oxopyridine-3-carboxylate (INT-A2-8). To a mixture of methyl 5-(1-cyclopropyl-5-hydroxypyrazol-4-yl)-1-methyl-6-oxopyridine-3-carboxylate (0.7 g, 2.42 mmol, 1 equiv) and triphenylphosphine (1.9 g, 7.26 mmol, 3 equiv) in tetrahydrofuran (20 mL) were added diisopropyl azodicarboxylate (1.4 g, 7.26 mmol, 3 equiv) and 2-({2-[(5-bromo-2-nitrophenyl)amino]ethyl}(2,2,2-trifluoroethyl)amino)ethanol (INT-A2-2, 0.9 g, 2.42 mmol, 1 equiv) at 0 °C under nitrogen atmosphere. The mixture was stirred for 2 h at 0 °C. The reaction was quenched with water (100 mL) and the mixture was extracted with ethyl acetate (3×100 mL). The combined organic layers were washed with brine (3×100 mL), dried over anhydrous Na₂SO₄, and concentrated under reduced pressure. The residue was purified by C18 Column using water containing 10 mmol/L NH₄HCO₃ and 0.1% NH₃—H₂O and acetonitrile as mobile phase. Fractions containing the desired compound were evaporated to dryness to give 400 mg of methyl 5-{5-[2-({2-[(5-bromo-2-nitrophenyl)amino]ethyl}(2,2,2-trifluoroethyl)amino)ethoxy]-1-cyclopropylpyrazol-4-yl}-1-methyl-6-oxopyridine-3-carboxylate (INT-A2-8) as an orange solid (25%). LCMS: m/z (ESI), [M+H]⁺=658.95. ¹H NMR (DMSO-d₆, 400 MHz) δ 0.92-1.03 (2H, m), 0.99-1.10 (2H, m), 2.51 (3H, d), 3.04 (2H, t), 3.17 (2H, t), 3.44 (2H, q), 3.55-3.60 (3H, m), 3.77 (3H, s), 4.12-4.15 (2H, m), 6.80-6.83 (1H, m), 7.23 (1H, d), 7.88 (1H, s), 7.94 (1H, d), 8.06 (1H, d), 8.25 (1H, t), 8.38 (1H, d).

Step 9. Preparation of methyl 5-{5-[2-({2-[(2-amino-5-bromophenyl)amino]ethyl}(2,2,2-trifluoroethyl)amino)ethoxy]-1-cyclopropylpyrazol-4-yl}-1-methyl-6-oxopyridine-3-carboxylate (INT-A2-9). To a mixture of methyl 5-{5-[2-({2-[(5-bromo-2-nitrophenyl)amino]ethyl}(2,2,2-trifluoroethyl)amino)ethoxy]-1-cyclopropylpyrazol-4-yl}-1-methyl-6-oxopyridine-3-carboxylate (350 mg, 0.53 mmol, 1 equiv) and Raney-Ni (365 mg, 4.26 mmol, 8 equiv) in methanol (5 mL) was added hydrazine hydrate (98%) (40 mg, 0.80 mmol, 1.5 equiv) at 0 °C under nitrogen atmosphere. The resulting mixture was stirred for 2 h and filtered. The filter cake was washed with methanol (3×50 mL) and the solution was concentrated under reduced pressure to give 300 mg of methyl 5-{5-[2-({2-[(2-amino-5-bromophenyl)amino]ethyl}(2,2,2-trifluoroethyl)amino)ethoxy]-1-cyclopropylpyrazol-4-yl}-1-methyl-6-oxopyridine-3-carboxylate (INT-A2-9) as a brown solid (~90%). LCMS: m/z (ESI), [M+H]⁺=627.15. ¹H NMR (DMSO-d₆, 400 MHz) δ 0.91-1.03 (2H, m), 0.99-1.10 (2H, m), 2.98 (2H, t), 3.13-3.19 (4H, m), 3.51 (2H, q), 3.57 (3H, s), 3.79 (3H, s), 4.01-4.07 (3H, m), 4.12 (2H, t), 5.76 (1H, s), 6.38-6.58 (3H, m), 7.91 (1H, d), 8.13 (1H, d), 8.45 (1H, d).

Step 10. Preparation of methyl 5-[5-(2-{[2-(2-amino-6-bromo-1,3-benzodiazol-1-yl)ethyl](2,2,2-trifluoroethyl)amino}ethoxy)-1-cyclopropylpyrazol-4-yl]-1-methyl-6-oxopyridine-3-carboxylate (INT-A2-10). A mixture of methyl 5-{5-[2-({2-[(2-amino-5-bromophenyl)amino]ethyl}(2,2,2-trifluoroethyl)amino)ethoxy]-1-cyclopropylpyrazol-4-yl}-1-methyl-6-oxopyridine-3-carboxylate (300 mg, 0.48 mmol, 1 equiv) and BrCN (60 mg, 0.57 mmol, 1.2 equiv) in dichloromethane (5 mL) was stirred for 2 h at room temperature under nitrogen atmosphere and concentrated under vacuum. The residue was purified on a prep-TLC eluted with dichloromethane/methanol (10/1) to give 300 mg of methyl 5-[5-(2-{[2-(2-amino-6-bromo-1,3-benzodiazol-1-yl)ethyl](2,2,2-trifluoroethyl)amino}ethoxy)-1-cyclopropylpyrazol-4-yl]-1-methyl-6-oxopyridine-3-carboxylate (INT-A2-10) as a brown solid (96%). LCMS: m/z (ESI), [M+H]⁺=654.10.

Step 11. Preparation of 5-[5-(2-{[2-(2-amino-6-bromo-1,3-benzodiazol-1-yl)ethyl](2,2,2-trifluoroethyl)amino}ethoxy)-1-cyclopropylpyrazol-4-yl]-1-methyl-6-oxopyridine-3-carboxylic acid (INT-A2-11). A mixture of methyl 5-[5-(2-{[2-(2-amino-6-bromo-1,3-benzodiazol-1-yl)ethyl](2,2,2-trifluoroethyl)amino}ethoxy)-1-cyclopropylpyrazol-4-yl]-1-methyl-6-oxopyridine-3-carboxylate (280 mg, 0.43 mmol, 1 equiv) and LiOH H₂O (27 mg, 0.64 mmol, 1.5 equiv) in THF (4 mL) and H₂O (1 mL) was stirred for 2 h at room temperature under nitrogen atmosphere and concentrated under reduced pressure. The residue was purified by prep-HPLC with XBridge Shield RP18 OBD Column using water containing 10 mmol/L NH₄HCO₃ and 0.10% NH₃—H₂O and acetonitrile as mobile phase. Fractions containing the desired compound were evaporated to dryness to give 135 mg of 5-[5-(2-{[2-(2-amino-6-bromo-1,3-benzodiazol-1-yl)ethyl](2,2,2-trifluoroethyl)amino}ethoxy)-1-cyclopropylpyrazol-4-yl]-1-methyl-6-oxopyridine-3-carboxylic acid (INT-A2-11) as a brown solid (49%). LCMS: m/z (ESI), [M+H]⁺=638.10. ¹H NMR (DMSO-d₆, 400 MHz) δ 0.90-1.03 (2H, m), 0.99-1.09 (2H, m), 3.01 (2H, t), 3.51 (3H, s), 3.53-3.63 (3H, m), 4.08 (2H, t), 4.23 (2H, t), 6.80-7.13 (3H, m), 7.19 (2H, s), 7.31 (1H, d), 7.97-8.05 (2H, m), 8.43 (1H, d)

Step 12. Preparation of 16-bromo-5-cyclopropyl-26-methyl-10-(2,2,2-trifluoroethyl)-7-oxa-4,5,10,13,20,22,26-heptaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (INT-A2-12). A mixture of 5-[5-(2-{[2-(2-amino-6-bromo-1,3-benzodiazol-1-yl)ethyl](2,2,2-trifluoroethyl)amino}ethoxy)-1-cyclopropylpyrazol-4-yl]-1-methyl-6-oxopyridine-3-carboxylic acid (110 mg, 0.17 mmol, 1 equiv), N,N-diisopropylethylamine (67 mg, 0.52 mmol, 3 equiv), and N,N,N,N-tetramethyl-O-(7-azabenzotriazol-1-yl)uronium hexafluorophospate (98 mg, 0.26 mmol, 1.5 equiv) in 1,4-dioxane (5 mL) was stirred for 2 h at room temperature under nitrogen atmosphere. The reaction was quenched with water (50 mL) and the mixture was extracted with ethyl acetate (3×50 mL). The combined organic layers were washed with brine (3×50 mL), dried over anhydrous Na₂SO₄, and concentrated under reduced pressure. The residue was purified by prep-HPLC with XBridge Shield RP18 OBD Column using water containing 10 mmol/L NH₄HCO₃ and 0.1% NH₃—H₂O and acetonitrile as mobile phase. Fractions containing the desired compound were evaporated to dryness to give 60 mg of 16-bromo-5-cyclopropyl-26-methyl-10-(2,2,2-trifluoroethyl)-7-oxa-4,5,10,13,20,22,26-heptaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (INT-A2-12) as a brown solid (56%). LCMS: m/z (EST), [M+H]⁺=622.05. ¹H NMR (DMSO-d₆, 400 MHz) δ 0.96-1.04 (2H, m), 1.08-1.35 (2H, m), 3.08-3.12 (2H, m), 3.28 (2H, q), 3.49-3.60 (1H, m), 3.62 (3H, s), 3.64-3.77 (2H, m), 4.21-4.33 (2H, m), 4.29 (2H, s), 7.36-7.38 (1H, m), 7.43 (1H, d), 8.09 (1H, d), 8.21 (1H, s), 8.30 (1H, d), 8.88 (1H, d), 12.63 (1H, s).

Step 13. Preparation of 5-cyclopropyl-26-methyl-16-(4-methylpiperazin-1-yl)-10-(2,2,2-trifluoroethyl)-7-oxa-4,5,10,13,20,22,26-heptaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (EXAMPLE A2). To a mixture of 16-bromo-5-cyclopropyl-26-methyl-10-(2,2,2-trifluoroethyl)-7-oxa-4,5,10,13,20,22,26-heptaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (40 mg, 0.06 mmol, 1 equiv), 1-methylpiperazine (26 mg, 0.256 mmol, 4 equiv), and BrettPhos Pd G₃ (18 mg, 0.02 mmol, 0.3 equiv) in 1,4-dioxane (5 mL) was added LiHMDS (65 mg, 0.38 mmol, 6 equiv) at room temperature under nitrogen atmosphere. The mixture was stirred for 2 h at 60 °C and then cooled to room temperature. The reaction mixture was treated with NH₄Cl (aq.) solution (50 mL) and extracted with ethyl acetate (3×50 mL). The combined organic layers were washed with brine (3×30 mL), dried over anhydrous Na₂SO₄, and concentrated under reduced pressure. The residue was purified by prep-HPLC with Shield RP18 OBD Column using water containing 10 mmol/L NH₄HCO₃ and 0.1% NH₃—H₂O and acetonitrile as mobile phase. Fractions containing the desired compound were evaporated to dryness to give 2.7 mg of 5-cyclopropyl-26-methyl-16-(4-methylpiperazin-1-yl)-10-(2,2,2-trifluoroethyl)-7-oxa-4,5,10,13,20,22,26-heptaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (EXAMPLE A2) as a white solid (6%). LCMS: m/z (EST), [M+H]⁺=640.35. ¹H NMR (DMSO-d₆, 400 MHz) δ 0.96-1.04 (2H, m), 1.08-1.35 (2H, m), 2.25 (3H, s), 2.48-2.50 (4H, m), 3.04-3.15 (3H, m), 3.18 (4H, t), 3.57-3.64 (4H, m), 3.62 (3H, s), 4.24-4.29 (4H, m), 6.87 (1H, d), 7.23 (1H, s), 7.35 (1H, d), 8.21 (1H, s), 8.27 (1H, s), 8.89 (1H, s), 12.33 (1H, s). ¹⁹F NMR (DMSO-d₆, 376 MHz) δ 69.26 (s).

### Example A3

### 5,26-Dimethyl-16-(4-methylpiperazin-1-yl)-10-(2,2,2-trifluoroethyl)-7-oxa-4,5,10,13,20,22,26-heptaazapentacyclo[22.3.1.0\hat{ }{2,6}.0\hat{ }{13,21}.0\hat{ }{14,19}]octacosa-1(28),2(6),3,14,16,18,20,24-octaene-23,27-dione

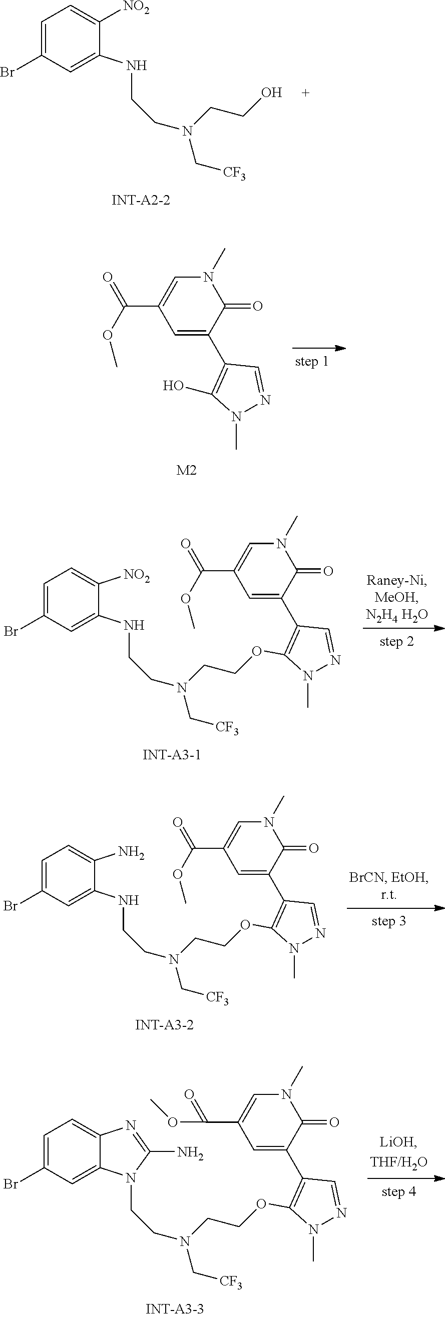 [SMILES: O=[N+]([O-])c1ccc(Br)cc1NCCN(CCO)CC(F)(F)F ; COC(=O)c1cc(-c2cnn(C)c2O)c(=O)n(C)c1 ; "M2" ; COC(=O)c1cc(-c2cnn(C)c2OCCN(CCNc2cc(Br)ccc2[N+](=O)[O-])CC(F)(F)F)c(=O)n(C)c1 ; COC(=O)c1cc(-c2cnn(C)c2OCCN(CCNc2cc(Br)ccc2N)CC(F)(F)F)c(=O)n(C)c1 ; COC(=O)c1cc(-c2cnn(C)c2OCCN(CCn2c(N)nc3ccc(Br)cc32)CC(F)(F)F)c(=O)n(C)c1]

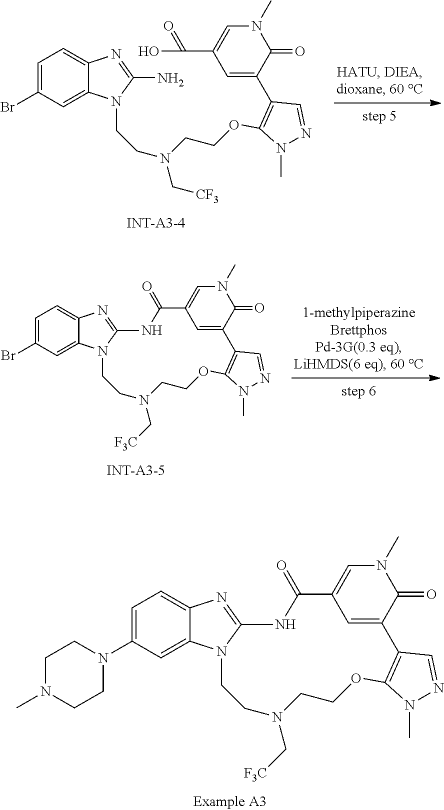 [SMILES: Cn1ncc(-c2cc(C(=O)O)cn(C)c2=O)c1OCCN(CCn1c(N)nc2ccc(Br)cc21)CC(F)(F)F ; Cn1ncc2c1OCCN(CC(F)(F)F)CCn1c(nc3ccc(Br)cc31)NC(=O)c1cc-2c(=O)n(C)c1 ; CN1CCN(c2ccc3nc4n(c3c2)CCN(CC(F)(F)F)CCOc2c(cnn2C)-c2cc(cn(C)c2=O)C(=O)N4)CC1]

Step 1. Preparation of methyl-5-{5-[2-({2-[(5-bromo-2-nitrophenyl)amino]ethyl}(2,2,2-trifluoroethyl)amino)ethoxy]-1-methylpyrazol-4-yl}-1-methyl-6-oxopyridine-3-carboxylate (INT-A3-1). To a stirred mixture of triphenylphosphine (1324 mg, 5.04 mmol, 3 equiv) and methyl 5-(5-hydroxy-1-methylpyrazol-4-yl)-1-methyl-6-oxopyridine-3-carboxylate (M2, 354 mg, 1.34 mmol, 0.8 equiv) in tetrahydrofuran (9 mL) were added 2-({2-[(5-bromo-2-nitrophenyl)amino]ethyl}(2,2,2-trifluoroethyl)amino)ethanol (650 mg, 1.68 mmol, 1 equiv) and diisopropylazodicarboxylate (1021 mg, 5.04 mmol, 3 equiv) at 0 °C. The mixture was stirred for 1 h at 0 °C under nitrogen atmosphere. The reaction was quenched with water (50 mL) and the mixture was extracted with ethyl acetate (3×50 mL). The combined organic layers were washed with brine (3×50 mL), dried over anhydrous Na₂SO₄, and concentrated under reduced pressure. The residue was purified by Prep-TLC eluting with dichloromethane/methanol (10/1) to give 550 mg of methyl 5-{5-[2-({2-[(5-bromo-2-nitrophenyl)amino]ethyl}(2,2,2-trifluoroethyl)amino)ethoxy]-1-methylpyrazol-4-yl}-1-methyl-6-oxopyridine-3-carboxylate (INT-A3-1) as a yellow oil (36%). LCMS: m/z (ESI), [M+H]⁺=630.85. ¹H NMR (DMSO-d₆, 400 MHz) δ 3.01 (2H, t), 3.11 (2H, t), 3.31-3.45 (2H, m) 3.49-3.54 (4H, m), 3.66 (3H, s), 3.76 (3H, s), 4.01 (2H, t), 5.73 (1H, s), 6.78-6.92 (1H, m), 7.20 (1H, d), 7.96-7.88 (2H, m), 8.00 (1H, d), 8.23 (1H, t), 8.33 (1H, d).

Step 2. Preparation of methyl 5-{5-[2-({2-[(2-amino-5-bromophenyl)amino]ethyl}(2,2,2-trifluoroethyl)amino)ethoxy]-1-methylpyrazol-4-yl}-1-methyl-6-oxopyridine-3-carboxylate (INT-A3-2). To a stirred mixture of methyl 5-{5-[2-({2-[(5-bromo-2-nitrophenyl)amino]ethyl}(2,2,2-trifluoroethyl)amino)ethoxy]-1-methylpyrazol-4-yl}-1-methyl-6-oxopyridine-3-carboxylate (570 mg, 0.90 mmol, 1 equiv) and Raney-Ni (30 mg) in methanol (6 mL) was added NH₂NH₂—H₂O (57 mg, 1.80 mmol, 2 equiv). The mixture was stirred for 30 min at 0 °C under nitrogen atmosphere. The mixture was filtered. The filter cake was washed with methanol (3×30 mL) and the solution was concentrated under reduced pressure to give 270 mg of methyl 5-{5-[2-({2-[(2-amino-5-bromophenyl)amino]ethyl}(2,2,2-trifluoroethyl)amino)ethoxy]-1-methylpyrazol-4-yl}-1-methyl-6-oxopyridine-3-carboxylate (INT-A3-2) as a yellow oil (42%). LCMS: m/z (ESI), [M+H]⁺=601.05.

Step 3. Preparation of methyl 5-[5-(2-{[2-(2-amino-6-bromo-1,3-benzodiazol-1-yl)ethyl](2,2,2-trifluoroethyl)amino}ethoxy)-1-methylpyrazol-4-yl]-1-methyl-6-oxopyridine-3-carboxylate (INT-A3-3). A mixture of methyl 5-{5-[2-({2-[(2-amino-5-bromophenyl)amino]ethyl}(2,2,2-trifluoroethyl)amino)ethoxy]-1-methylpyrazol-4-yl}-1-methyl-6-oxopyridine-3-carboxylate (240 mg, 0.39 mmol, 1 equiv) and BrCN (42 mg, 0.39 mmol, 1 equiv) in ethanol (6 mL) was stirred for 1 h at 0 °C under nitrogen atmosphere. The mixture was concentrated under reduced pressure. The residue was purified by Prep-TLC eluted with dichloromethane/methanol (10/1) to give 120 mg of methyl 5-[5-(2-{[2-(2-amino-6-bromo-1,3-benzodiazol-1-yl)ethyl](2,2,2-trifluoroethyl)amino}ethoxy)-1-methylpyrazol-4-yl]-1-methyl-6-oxopyridine-3-carboxylate (INT-A3-3) as a brown solid (43%). LCMS: m/z (ESI), [M+H]⁺=626.05

Step 4. Preparation of 5-[5-(2-{[2-(2-amino-6-bromo-1,3-benzodiazol-1-yl)ethyl](2,2,2-trifluoroethyl)amino}ethoxy)-1-methylpyrazol-4-yl]-1-methyl-6-oxopyridine-3-carboxylic acid (INT-A3-4). To a stirred mixture of methyl 5-[5-(2-{[2-(2-amino-6-bromo-1,3-benzodiazol-1-yl)ethyl](2,2,2-trifluoroethyl)amino}ethoxy)-1-methylpyrazol-4-yl]-1-methyl-6-oxopyridine-3-carboxylate (100 mg, 0.16 mmol, 1 equiv) in tetrahydrofuran (2 mL) was added LiOH·H₂O (7.6 mg, 0.32 mmol, 2 equiv) in H₂O (0.5 mL). The resulting mixture was stirred for 2 h at room temperature under nitrogen atmosphere. The mixture was concentrated under reduced pressure. The residue was purified by C18 column using water and acetonitrile as mobile phase. Fractions containing the desired compound were evaporated to dryness to give 70 mg of 5-[5-(2-{[2-(2-amino-6-bromo-1,3-benzodiazol-1-yl)ethyl](2,2,2-trifluoroethyl)amino}ethoxy)-1-methylpyrazol-4-yl]-1-methyl-6-oxopyridine-3-carboxylic acid (INT-A3-4) as a white solid (68%). LCMS: m/z (ESI), [M+H]⁺=612.00. ¹H NMR (DMSO-d₆, 400 MHz) δ 2.98 (2H, t), 3.51 (3H, s), 3.57 (5H, t), 3.68 (3H, s), 3.99 (2H, t), 4.25 (2H, t), 7.06-6.97 (2H, m), 7.28 (2H, d), 7.33 (3H, s), 7.98 (1H, d), 8.09 (1H, s), 8.47 (1H, d,).

Step 5. Preparation of 16-bromo-5,26-dimethyl-10-(2,2,2-trifluoroethyl)-7-oxa-4,5,10,13,20,22,26-heptaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (INT-A3-5). To a stirred mixture of 5-[5-(2-{[2-(2-amino-6-bromo-1,3-benzodiazol-1-yl)ethyl](2,2,2-trifluoroethyl)amino}ethoxy)-1-methylpyrazol-4-yl]-1-methyl-6-oxopyridine-3-carboxylic acid (65 mg, 0.10 mmol, 1 equiv) and N,N,N,N-tetramethyl-O-(7-azabenzotriazol-1-yl)uronium hexafluorophospate (60 mg, 0.15 mmol, 1.5 equiv) in 1,4-dioxane (2 mL) was added N,N-Diisopropylethylamine (41 mg, 0.31 mmol, 3 equiv) and the mixture was stirred for 2 h at 60 °C under nitrogen atmosphere. After being cooled to room temperature, the mixture was concentrated under reduced pressure. The residue was purified by Prep-TLC eluted with dichloromethane/methanol (10/1) to give 45 mg of 16-bromo-5,26-dimethyl-10-(2,2,2-trifluoroethyl)-7-oxa-4,5,10,13,20,22,26-heptaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (INT-A3-5) as a brown solid (58%). LCMS: m/z (ESI), [M+H]⁺=594.00.

Step 6. Preparation of 5,26-dimethyl-16-(4-methylpiperazin-1-yl)-10-(2,2,2-trifluoroethyl)-7-oxa-4,5,10,13,20,22,26-heptaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (Example A3). To a stirred mixture of 16-bromo-5,26-dimethyl-10-(2,2,2-trifluoroethyl)-7-oxa-4,5,10,13,20,22,26-heptaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (50 mg, 0.08 mmol, 1 equiv), BrettPhos Pd G₃ (22 mg, 0.02 mmol, 0.3 equiv), and piperazine, 1-methyl- (33 mg, 0.33 mmol, 4 equiv) in 1,4-dioxane (2 mL) was added LiHMDS (0.48 mL, 0.48 mmol, 6 equiv) dropwise at room temperature under nitrogen atmosphere. The mixture was stirred for 40 min at 60 °C under nitrogen atmosphere. The mixture was cooled to room temperature. The reaction was quenched with water (30 mL) and the mixture was extracted with ethyl acetate (3×20 mL).

The combined organic layers were washed with brine (3×30 mL), dried over anhydrous Na₂SO₄, and concentrated under reduced pressure. The residue was purified by Prep-HPLC with XBridge Shield RP18 OBD column using water containing 10 mmol/L NH₄HCO₃ and 0.10% NH₃·H₂O and acetonitrile as mobile phase. Fractions containing the desired compound were evaporated to dryness to give 3.1 mg of 5,26-dimethyl-16-(4-methylpiperazin-1-yl)-10-(2,2,2-trifluoroethyl)-7-oxa-4,5,10,13,20,22,26-heptaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (Example A3) as a yellow solid (5%). LCMS: m/z (ESI), [M+H]⁺=614.25. ¹H NMR (DMSO-d₆, 400 MHz) δ 2.26 (3H, s), 3.04-3.10 (2H, m), 3.18 (4H, t), 3.15-3.27 (5H, m), 3.58-3.64 (4H, m), 3.64-3.78 (5H, m), 4.17 (2H, t), 4.29 (2H, t), 6.85-6.89 (1H, m), 7.23 (1H, d), 7.35 (1H, d), 8.26 (2H, d), 8.88 (1H, d), 12.34 (1H, s). ¹⁹F NMR (DMSO-d₆, 376 MHz) δ −69.31 (s).

### Example A4

### 10-(2,2-Difluoroethyl)-16-{[2-(dimethylamino)ethyl](methyl)amino}-5,26-dimethyl-7-oxa-4,5,10,13,20,22,26-heptaazapentacyclo[22.3.1.0\hat{ }{2,6}.0\hat{ }{13,21}.0\hat{ }{14,19}]octacosa-1(28),2(6),3,14,16,18,20,24-octaene-23,27-dione

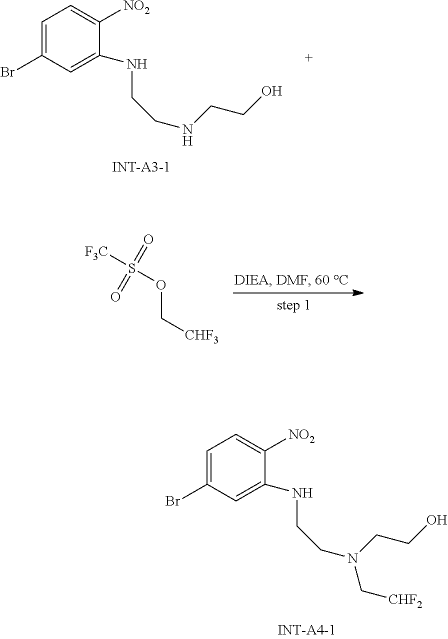

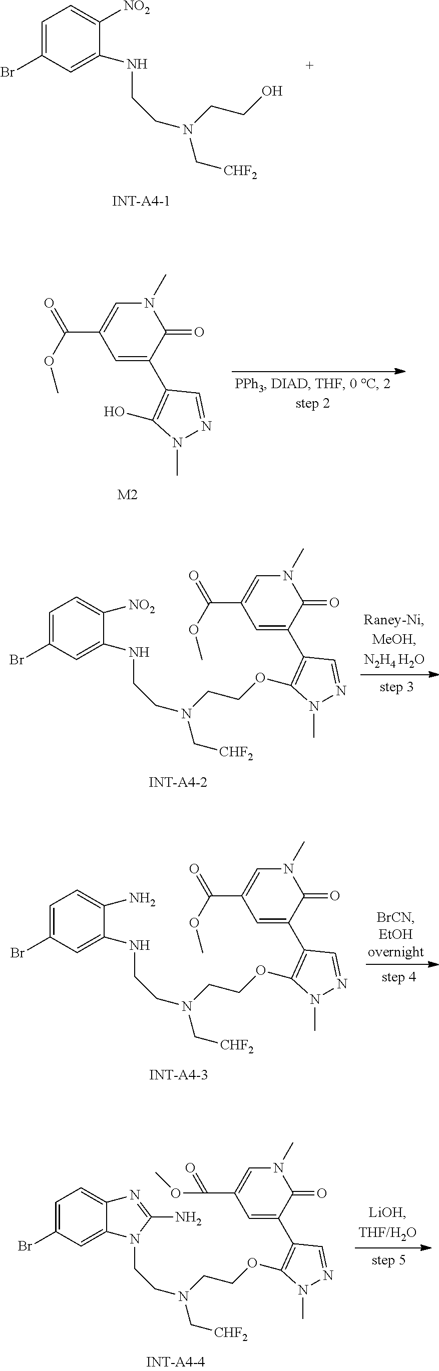 [SMILES: O=[N+]([O-])c1ccc(Br)cc1NCCN(CCO)CC(F)F ; COC(=O)c1cc(-c2cnn(C)c2O)c(=O)n(C)c1 ; "M2" ; COC(=O)c1cc(-c2cnn(C)c2OCCN(CCNc2cc(Br)ccc2[N+](=O)[O-])CC(F)F)c(=O)n(C)c1 ; COC(=O)c1cc(-c2cnn(C)c2OCCN(CCNc2cc(Br)ccc2N)CC(F)F)c(=O)n(C)c1 ; COC(=O)c1cc(-c2cnn(C)c2OCCN(CCn2c(N)nc3ccc(Br)cc32)CC(F)F)c(=O)n(C)c1]

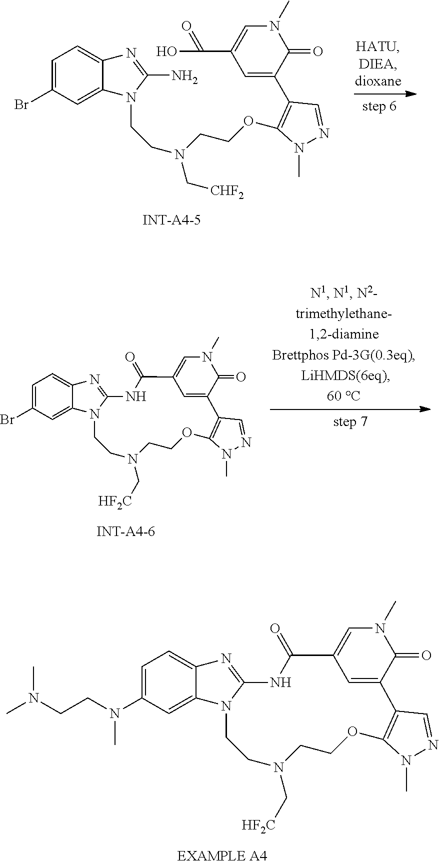

Step 1. Preparation of 2-({2-[(5-bromo-2-nitrophenyl)amino]ethyl}(2,2-difluoroethyl)amino)ethanol (INT-A4-1). A mixture of 2-({2-[(5-bromo-2-nitrophenyl)amino]ethyl}amino)ethanol (2 g, 6.57 mmol, 1 equiv), N,N-Diisopropylethylamine (2.55 g, 19.72 mmol, 3 equiv) and 2,2-difluoroethyl trifluoromethanesulfonate (1.69 g, 7.89 mmol, 1.2 equiv) in dimthylformamide (20 mL) was stirred for 2 h at 60 °C under nitrogen atmosphere. The mixture was cooled to room temperature. The reaction was quenched with water (80 mL) and the mixture was extracted with ethyl acetate (3×60 mL). The combined organic layers were washed with brine (3×50 mL), dried over anhydrous Na₂SO₄, and concentrated under reduced pressure. The residue was purified by Prep-TLC eluted with dichloromethane/methanol (10/1) to give 1.1 g of 2-({2-[(5-bromo-2-nitrophenyl)amino]ethyl}(2,2-difluoroethyl)amino)ethanol (INT-A4-1) as a yellow oil (43%). LCMS: m/z (EST), [M+H]⁺=366.00. ¹H NMR (DMSO-d₆, 400 MHz) δ 2.71 (2H, t), 2.90 (2H, t), 2.99 (2H, t), 3.35-3.43 (2H, m), 3.46-3.64 (2H, m), 4.51 (1H, t), 5.87-6.18 (1H, m), 6.83-6.86 (1H, m), 7.27 (1H, d), 7.99 (1H, d), 8.33 (1H, t).

Step 2. Preparation of methyl 5-{5-[2-({2-[(5-bromo-2-nitrophenyl)amino]ethyl}(2,2-difluoroethyl)amino)ethoxy]-1-methylpyrazol-4-yl}-1-methyl-6-oxopyridine-3-carboxylate (INT-A4-2). To a mixture of triphenylphosphine (1.38 g, 5.29 mmol, 3 equiv) and methyl 5-(5-hydroxy-1-methylpyrazol-4-yl)-1-methyl-6-oxopyridine-3-carboxylate (M2, 836 mg, 3.17 mmol, 1.8 equiv) in tetrahydrofuran (10 mL) were added 2-({2-[(5-bromo-2-nitrophenyl)amino]ethyl}(2,2-difluoroethyl)amino)ethanol (650 mg, 1.76 mmol, 1 equiv) and diisopropylazodicarboxylate (1.07 g, 5.29 mmol, 3 equiv) at 0 °C. The mixture was stirred for 1 h at 0 °C under nitrogen atmosphere. The reaction was quenched with water (80 mL) and the mixture was extracted with ethyl acetate (3×60 mL). The combined organic layers were washed with brine (3×50 mL), dried over anhydrous Na₂SO₄, and concentrated under reduced pressure. The residue was purified by Prep-TLC eluted with petroleum ether/ethyl acetate (1/3) to give 400 mg of methyl 5-{5-[2-({2-[(5-bromo-2-nitrophenyl)amino]ethyl}(2,2-difluoroethyl)amino)ethoxy]-1-methylpyrazol-4-yl}-1-methyl-6-oxopyridine-3-carboxylate (INT-A4-2) as a yellow solid (33%). LCMS: m/z (ESI), [M+H]⁺=613.00.

Step 3. Preparation of methyl 5-{5-[2-({2-[(2-amino-5-bromophenyl)amino]ethyl}(2,2-difluoroethyl)amino)ethoxy]-1-methylpyrazol-4-yl}-1-methyl-6-oxopyridine-3-carboxylate (INT-A4-3). To a stirred mixture of methyl 5-{5-[2-({2-[(5-bromo-2-nitrophenyl)amino]ethyl}(2,2-difluoroethyl)amino)ethoxy]-1-methylpyrazol-4-yl}-1-methyl-6-oxopyridine-3-carboxylate (400 mg, 0.65 mmol, 1 equiv) and Raney-Ni (72 mg) in methanol (5 mL) was added hydrazine (41 mg, 1.30 mmol, 2 equiv) at 0 °C. The mixture was stirred for 30 min at 0 °C under nitrogen atmosphere. The resulting mixture was filtered. The filter cake was washed with methanol (3×10 mL) and the solution was concentrated under reduced pressure to give 300 mg methyl-5-{5-[2-({2-[(2-amino-5-bromophenyl)amino]ethyl}(2,2-difluoroethyl)amino)ethoxy]-1-methylpyrazol-4-yl}-1-methyl-6-oxopyridine-3-carboxylate (INT-A4-3) as a brown solid (55%). LCMS: m/z (ESI), [M+H]+=583.05.

Step 4. Preparation of methyl 5-[5-(2-{[2-(2-amino-6-bromo-1,3-benzodiazol-1-yl)ethyl](2,2-difluoroethyl)amino}ethoxy)-1-methylpyrazol-4-yl]-1-methyl-6-oxopyridine-3-carboxylate (INT-A4-4). A mixture of methyl 5-{5-[2-({2-[(2-amino-5-bromophenyl)amino]ethyl}(2,2-difluoroethyl)amino)ethoxy]-1-methylpyrazol-4-yl}-1-methyl-6-oxopyridine-3-carboxylate (300 mg, 0.51 mmol, 1 equiv) and BrCN (54 mg, 0.51 mmol, 1 equiv) in ethanol (5 mL) was stirred for 1 h at room temperature under nitrogen atmosphere. The mixture was concentrated under reduced pressure. The residue was purified by Prep-TLC eluted with dichloromethane/methanol (10/1) to give 250 mg of methyl 5-[5-(2-{[2-(2-amino-6-bromo-1,3-benzodiazol-1-yl)ethyl](2,2-difluoroethyl)amino}ethoxy)-1-methylpyrazol-4-yl]-1-methyl-6-oxopyridine-3-carboxylate (INT-A4-4) as a brown solid (71%). LCMS: m/z (ESI), [M+H]+=608.05.

Step 5. Preparation of 5-[5-(2-{[2-(2-amino-6-bromo-1,3-benzodiazol-1-yl)ethyl](2,2-difluoroethyl)amino}ethoxy)-1-methylpyrazol-4-yl]-1-methyl-6-oxopyridine-3-carboxylic acid (INT-A4-5). To a stirred mixture of methyl 5-[5-(2-{[2-(2-amino-6-bromo-1,3-benzodiazol-1-yl)ethyl](2,2-difluoroethyl)amino}ethoxy)-1-methylpyrazol-4-yl]-1-methyl-6-oxopyridine-3-carboxylate (200 mg, 0.32 mmol, 1 equiv) in tetrahydrofuran (4 mL) was added LiOH·H₂O (19.6 mg, 0.82 mmol, 2.5 equiv) in H₂O (1 mL). The resulting mixture was stirred for 2 h at room temperature. The mixture was concentrated under reduced pressure. The residue was purified by Cl18 FLASH using water containing 10 mmol/L NH₄HCO₃ and 0.10% NH₃—H₂O and acetonitrile as mobile phase. Fractions containing the desired compound were evaporated to dryness to give 175 mg of 5-[5-(2-{[2-(2-amino-6-bromo-1,3-benzodiazol-1-yl)ethyl](2,2-difluoroethyl)amino}ethoxy)-1-methylpyrazol-4-yl]-1-methyl-6-oxopyridine-3-carboxylic acid (INT-A4-5) as a brown solid (80%). LCMS: m/z (ESI), [M+H]⁺=594.10. ¹H NMR (DMSO-d₆, 400 MHz) δ 2.90 (4H, t), 3.05 (4H, t), 3.27 (2H, d), 3.51 (6H, s), 3.68 (6H, s), 3.97 (4H, t), 4.24 (4H, t), 5.85-6.20 (1H, m), 7.06-6.97 (4H, m), 7.28 (4H, s), 7.32 (2H, d), 7.97 (2H, d), 8.08 (2H, s), 8.46 (1H, s), 8.47 (1H, s).

Step 6. Preparation of 16-Bromo-10-(2,2-difluoroethyl)-5,26-dimethyl-7-oxa-4,5,10,13,20,22,26-heptaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (INT-A4-6). A mixture of 5-[5-(2-{[2-(2-amino-6-bromo-1,3-benzodiazol-1-yl)ethyl](2,2-difluoroethyl)amino}ethoxy)-1-methylpyrazol-4-yl]-1-methyl-6-oxopyridine-3-carboxylic acid (160 mg, 0.26 mmol, 1 equiv), N,N,N,N-tetramethyl-O-(7-azabenzotriazol-1-yl)uronium hexafluorophospate (153 mg, 0.40 mmol, 1.5 equiv), and N,N-diisopropylethylamine (104 mg, 0.80 mmol, 3 equiv) in 1,4-dioxane (6 mL) was stirred for 2 h at 60 °C under nitrogen atmosphere. The mixture was cooled to room temperature. The reaction was quenched with water (30 mL) and the mixture was extracted with ethyl acetate (3×20 mL). The combined organic layers were washed with brine (3×20 mL), dried over anhydrous Na₂SO₄, and concentrated under reduced pressure. The residue was purified by Prep-TLC eluted with dichloromethane/methanol (10/1) to give 130 mg of 16-bromo-10-(2,2-difluoroethyl)-5,26-dimethyl-7-oxa-4,5,10,13,20,22,26-heptaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (INT-A4-6) as a brown solid (79%). LCMS(EB237475-165): m/z (ESI), [M+H]+=576.00;

Step 7. Preparation of 10-(2,2-Difluoroethyl)-16-{[2-(dimethylamino)ethyl](methyl)amino}-5,26-dimethyl-7-oxa-4,5,10,13,20,22,26-heptaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (EXAMPLE A4). To a mixture of 16-bromo-10-(2,2-difluoroethyl)-5,26-dimethyl-7-oxa-4,5,10,13,20,22,26-heptaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (100 mg, 0.17 mmol, 1 equiv), BrettPhos Pd G₃ (47 mg, 0.05 mmol, 0.3 equiv), and [2-(dimethylamino)ethyl](methyl)amine (70 mg, 0.69 mmol, 4 equiv) in 1,4-dioxane (3 mL) was added LiHMDS (0.7 mL, 1.02 mmol, 6 equiv) dropwise at room temperature under nitrogen atmosphere. The mixture was stirred for 2 h at 60 °C under nitrogen atmosphere and then cooled to room temperature. The reaction was quenched with sat. NH₄Cl (aq.) solution (5 mL) and the mixture was extracted with ethyl acetate (3×200 mL). The combined organic layers were washed with brine (3×300 mL), dried over anhydrous Na₂SO₄, and concentrated under reduced pressure. The residue was purified by prep-HPLC with XBridge Shield RP18 OBD column using water containing 10 mmol/L NH₄HCO₃+0.1% NH₃·H₂O and acetonitrile as mobile phase. Fractions containing the desired compound were evaporated to dryness to give 1.2 mg of 10-(2,2-difluoroethyl)-16-{[2-(dimethylamino)ethyl](methyl)amino}-5,26-dimethyl-7-oxa-4,5,10,13,20,22,26-heptaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (EXAMPLE A4) as a yellow solid (1%). LCMS: m/z (ESI), [M+H]⁺=598.25. ¹H NMR (DMSO-d₆, 400 MHz) δ 2.19 (6H, s), 2.39 (2H, t), 2.96 (3H, s), 3.03 (2H, t), 3.11-3.52 (6H, m), 3.61 (3H, s), 3.73 (3H, s), 4.19 (2H, t), 4.28 (2H, t), 6.21 (1H, t), 6.64 (1H, d), 6.84 (1H, s), 7.31 (1H, d), 8.25 (2H, s), 8.87 (1H, s), 12.20 (1H, s). ¹⁹F NMR (DMSO-d₆, 376 MHz) δ 119.603 (s).

### Example A5

### 5,26-Dimethyl-10-(oxetan-3-yl)-7-oxa-4,5,10,13,20,22,26-heptaazapentacyclo[22.3.1.0\hat{ }{2,6}.0\hat{ }{13,21}.0\hat{ }{14,19}]octacosa-1(28),2(6),3,14,16,18,20,24-octaene-23,27-dione

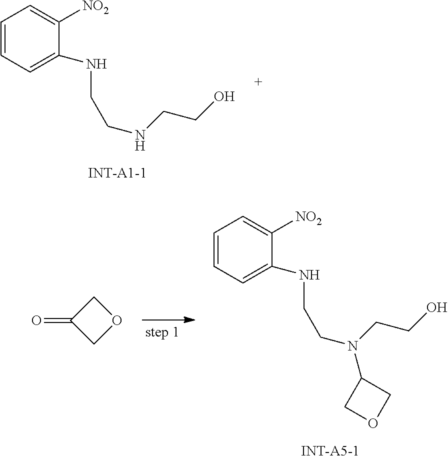 [SMILES: O=[N+]([O-])c1ccccc1NCCNCCO ; O=C1COC1 ; O=[N+]([O-])c1ccccc1NCCN(CCO)C1COC1]

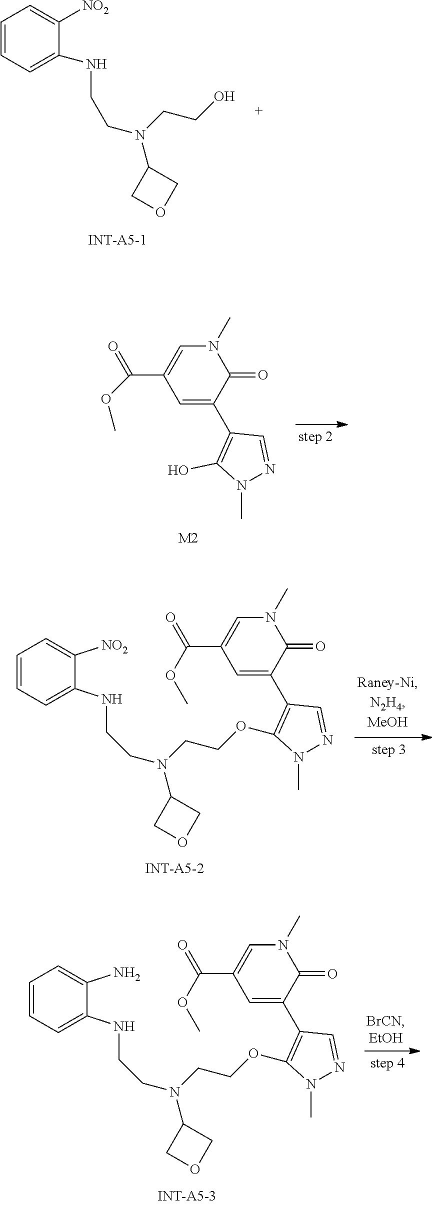 [SMILES: O=[N+]([O-])c1ccccc1NCCN(CCO)C1COC1 ; COC(=O)c1cc(-c2cnn(C)c2O)c(=O)n(C)c1 ; "M2" ; COC(=O)c1cc(-c2cnn(C)c2OCCN(CCNc2ccccc2[N+](=O)[O-])C2COC2)c(=O)n(C)c1 ; COC(=O)c1cc(-c2cnn(C)c2OCCN(CCNc2ccccc2N)C2COC2)c(=O)n(C)c1]

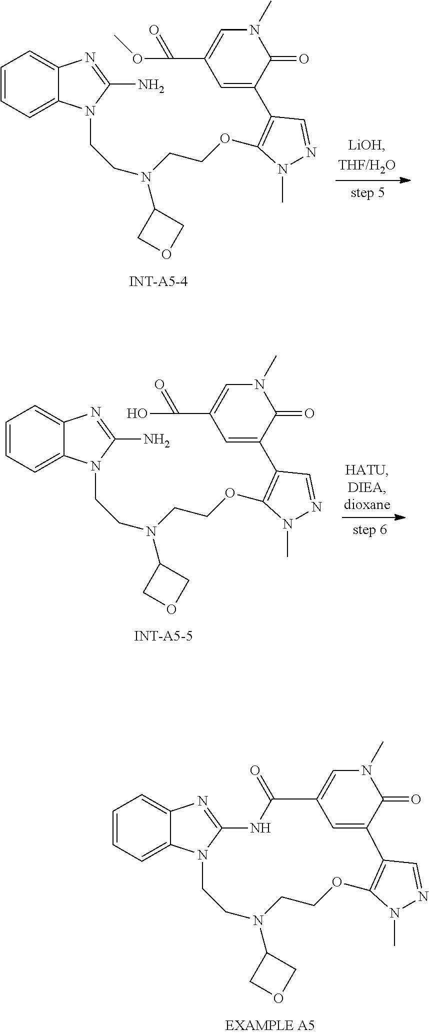 [SMILES: COC(=O)c1cc(-c2cnn(C)c2OCCN(CCn2c(N)nc3ccccc32)C2COC2)c(=O)n(C)c1 ; Cn1ncc(-c2cc(C(=O)O)cn(C)c2=O)c1OCCN(CCn1c(N)nc2ccccc21)C1COC1 ; Cn1ncc2c1OCCN(C1COC1)CCn1c(nc3ccccc31)NC(=O)c1cc-2c(=O)n(C)c1]

Step 1. Preparation of 2-({2-[(2-nitrophenyl)amino]ethyl}(oxetan-3-yl)amino)ethanol (INT-A5-1). To a stirred mixture of 2-({2-[(2-nitrophenyl)amino]ethyl}amino)ethanol (1 g, 4.44 mmol, 1 equiv), 3-oxetanone (383 mg, 5.33 mmol, 1.2 equiv) and molecular sieves (20 mg) in dichloromethane (30 mL) was added sodium triacetoxyborohydride (1.88 g, 8.88 mmol, 2 equiv) at room temperature. The mixture was stirred for 12 h at room temperature under nitrogen atmosphere. The reaction was quenched with water (80 mL) and the mixture was extracted with ethyl acetate (3×60 mL). The combined organic layers were washed with brine (3×50 mL), dried over anhydrous Na₂SO₄, and concentrated under reduced pressure. The residue was purified by Prep-TLC eluted with petroleum ether/ethyl acetate (1/1) to give 1.2 g of 2-({2-[(2-nitrophenyl)amino]ethyl}(oxetan-3-yl)amino)ethanol (INT-A5-1) as a red oil (95%). LCMS: m/z (EST), [M+H]⁺=282.10. ¹H NMR (DMSO-d₆, 400 MHz) δ 2.56-2.65 (2H, m), 2.74-2.81 (2H, m), 3.31-3.40 (2H, m), 3.41-3.49 (2H, m), 4.00-4.06 (1H, m), 4.42-4.48 (2H, m), 4.51-4.58 (2H, m), 6.64-6.74 (1H, m), 7.01-7.08 (1H, m), 7.50-7.59 (1H, m), 8.03-8.11 (1H, m), 8.32 (1H, t).

Step 2. Preparation of Methyl 1-methyl-5-{1-methyl-5-[2-({2-[(2-nitrophenyl)amino]ethyl}(oxetan-3-yl)amino)ethoxy]pyrazol-4-yl}-6-oxopyridine-3-carboxylate (INT-A5-2). To a stirred mixture of methyl 5-(5-hydroxy-1-methylpyrazol-4-yl)-1-methyl-6-oxopyridine-3-carboxylate (308 mg, 1.17 mmol, 1.1 equiv) and triphenyl phosphine (839 mg, 3.20 mmol, 3 equiv) in tetrahydrofuran (2 mL) were added diisopropyl azodicarboxylate (647 mg, 3.20 mmol, 3 equiv) and 2-({2-[(2-nitrophenyl)amino]ethyl}(oxetan-3-yl)amino)ethanol (300 mg, 1.07 mmol, 1 equiv) at 0 °C. The mixture was stirred for 12 h at room temperature under nitrogen atmosphere. The reaction was quenched with water (50 mL) and the mixture was extracted with ethyl acetate (3×30 mL). The combined organic layers were washed with brine (3×30 mL), dried over anhydrous Na₂SO₄, and concentrated under reduced pressure. The residue was purified by Prep-TLC eluted with petroleum ether/ethyl acetate (1/1) to give 200 mg of methyl 1-methyl-5-{1-methyl-5-[2-({2-[(2-nitrophenyl)amino]ethyl}(oxetan-3-yl)amino)ethoxy]pyrazol-4-yl}-6-oxopyridine-3-carboxylate (INT-A5-2) as a yellow solid (30%). LCMS: m/z (ESI), [M+H]⁺=527.20. ¹H NMR (DMSO-dd, 400 MHz) δ 2.79-2.87 (2H, m), 2.94-3.00 (2H, m), 3.29-3.40 (5H, m), 3.78 (3H, s), 3.94-4.03 (2H, m), 4.04-4.16 (1H, m), 4.42-4.51 (2H, m), 4.52-4.60 (2H, m), 6.64-6.73 (1H, m), 6.94-7.01 (1H, m), 7.45-7.53 (1H, m), 7.91 (1H, s), 8.01-8.08 (2H, m), 8.25 (1H, t), 8.39 (1H, d).

Step 3. Preparation of methyl 5-{5-[2-({2-[(2-aminophenyl)amino]ethyl}(oxetan-3-yl)amino)ethoxy]-1-methylpyrazol-4-yl}-1-methyl-6-oxopyridine-3-carboxylate (INT-A5-3). To a stirred mixture of methyl 1-methyl-5-{1-methyl-5-[2-({2-[(2-nitrophenyl)amino]ethyl}(oxetan-3-yl)amino)ethoxy]pyrazol-4-yl}-6-oxopyridine-3-carboxylate (150 mg, 0.29 mmol, 1 equiv) and Raney Ni (30 mg, 0.35 mmol, 1.2 equiv) in methanol (30 mL) was added NH₂NH₂·H₂O (90 mg) at 0 °C. The mixture was stirred for 1 h at 0 °C under nitrogen atmosphere. The resulting mixture was filtered. The filter cake was washed with methanol (2×10 mL) and the solution was concentrated under reduced pressure. The residue was purified by Prep-TLC eluted with dichloromethane/methanol (10/1) to give 120 mg of methyl 5-{5-[2-({2-[(2-aminophenyl)amino]ethyl}(oxetan-3-yl)amino)ethoxy]-1-methylpyrazol-4-yl}-1-methyl-6-oxopyridine-3-carboxylate (INT-A5-3) as a yellow solid (73%). LCMS: m/z (ESI), [M+H]⁺=497.30.

Step 4. Preparation of methyl 5-[5-(2-{[2-(2-amino-1,3-benzodiazol-1-yl)ethyl](oxetan-3-yl)amino}ethoxy)-1-methylpyrazol-4-yl]-1-methyl-6-oxopyridine-3-carboxylate (INT-A5-4). A mixture of methyl 5-{5-[2-({2-[(2-aminophenyl)amino]ethyl}(oxetan-3-yl)amino)ethoxy]-1-methylpyrazol-4-yl}-1-methyl-6-oxopyridine-3-carboxylate (120 mg, 0.24 mmol, 1 equiv) and cyanogen bromide (38 mg, 0.36 mmol, 1.5 equiv) in ethanol (10 mL) was stirred for 2 h at room temperature under nitrogen atmosphere. The mixture was concentrated under reduced pressure. The residue was purified by Prep-TLC eluted with dichloromethane/methanol (5/1) to give 82 mg of methyl 5-[5-(2-{[2-(2-amino-1,3-benzodiazol-1-yl)ethyl](oxetan-3-yl)amino}ethoxy)-1-methylpyrazol-4-yl]-1-methyl-6-oxopyridine-3-carboxylate (INT-A5-4) as a yellow solid (65%). LCMS: m/z (EST), [M+H]⁺=522.25. ¹H NMR (DMSO-d₆, 400 MHz) δ 2.77-2.86 (2H, m), 2.99-3.07 (2H, m), 3.57 (3H, s), 3.68 (3H, s), 3.77 (3H, s), 3.91-3.98 (2H, m), 3.98-4.11 (3H, m), 4.29-4.36 (2H, m), 4.43-4.50 (2H, m), 6.39 (2H, s), 6.78-6.85 (1H, m), 6.87-6.95 (1H, m), 7.04-7.14 (1H, m), 7.93 (1H, s), 8.08 (1H, d), 8.45 (1H, d).

Step 5. Preparation of 5-[5-(2-{[2-(2-amino-1,3-benzodiazol-1-yl)ethyl](oxetan-3-yl)amino}ethoxy)-1-methylpyrazol-4-yl]-1-methyl-6-oxopyridine-3-carboxylic acid (INT-A5-5). A mixture of methyl 5-[5-(2-{[2-(2-amino-1,3-benzodiazol-1-yl)ethyl](oxetan-3-yl)amino}ethoxy)-1-methylpyrazol-4-yl]-1-methyl-6-oxopyridine-3-carboxylate (82 mg, 0.16 mmol, 1 equiv) and lithium hydroxide monohydrate (13 mg, 0.31 mmol, 2 equiv) in tetrahydrofuran (10 mL) and water (2.5 mL) was stirred for 2 h at room temperature under nitrogen atmosphere. The mixture was concentrated under reduced pressure. The residue was purified by C18 FLASH using water containing 10 mmol/L NH₄HCO₃ and 0.1% NH₃·H₂O and acetonitrile as mobile phase. Fractions containing the desired compound were evaporated to dryness to give 20 mg of 5-[5-(2-{[2-(2-amino-1,3-benzodiazol-1-yl)ethyl](oxetan-3-yl)amino}ethoxy)-1-methylpyrazol-4-yl]-1-methyl-6-oxopyridine-3-carboxylic acid (INT-A5-5) as a red solid (24%). LCMS: m/z (ESI), [M+H]⁺=508.25.

Step 6. Preparation of 5,26-dimethyl-10-(oxetan-3-yl)-7-oxa-4,5,10,13,20,22,26-heptaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (EXAMPLE A5). A mixture of 5-[5-(2-{[2-(2-amino-1,3-benzodiazol-1-yl)ethyl](oxetan-3-yl)amino}ethoxy)-1-methylpyrazol-4-yl]-1-methyl-6-oxopyridine-3-carboxylic acid (20 mg, 0.04 mmol, 1 equiv), N,N-Diisopropylethylamine (15 mg, 0.12 mmol, 3 equiv) and N,N,N,N-tetramethyl-O-(7-azabenzotriazol-1-yl)uronium hexafluorophospate (22 mg, 0.06 mmol, 1.5 equiv) in 1,4-dioxane (4 mL) was stirred for 3 h at room temperature under nitrogen atmosphere. The mixture was concentrated under reduced pressure. The residue was purified by prep-HPLC with XBridge Prep OBD C18 column using water containing 10 mmol/L NH₄HCO₃ and 0.1% NH₃—H₂O and acetonitrile as mobile phase. Fractions containing the desired compound were evaporated to dryness to give 8.8 mg of 5,26-dimethyl-10-(oxetan-3-yl)-7-oxa-4,5,10,13,20,22,26-heptaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (EXAMPLE A5) as a white solid (45%). LCMS: m/z (ESI), [M+H]⁺=490.20. ¹H NMR (DMSO-d₆, 400 MHz) δ 2.95-3.03 (2H, m), 3.17-3.24 (2H, m), 3.62 (3H, s), 3.74 (3H, s), 4.13-4.30 (5H, m), 4.59-4.64 (2H, m), 4.66-4.72 (2H, m), 7.19-7.29 (2H, m), 7.49-7.54 (1H, m), 7.58-7.64 (1H, m), 8.24 (1H, s), 8.29 (1H, d), 8.80 (1H, d), 11.69 (1H, s).

### Example A6

### 10-Ethyl-5,12,26-trimethyl-7-oxa-4,5,10,13,20,22,26-heptaazapentacyclo[22.3.1.0\hat{ }{2,6}.0\hat{ }{13,21}.0\hat{ }{14,19}]octacosa-1(28),2(6),3,14,16,18,20,24-octaene-23,27-dione

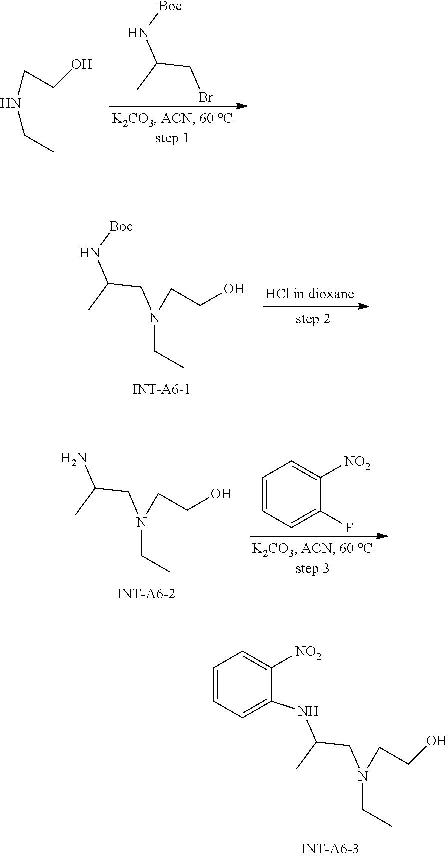

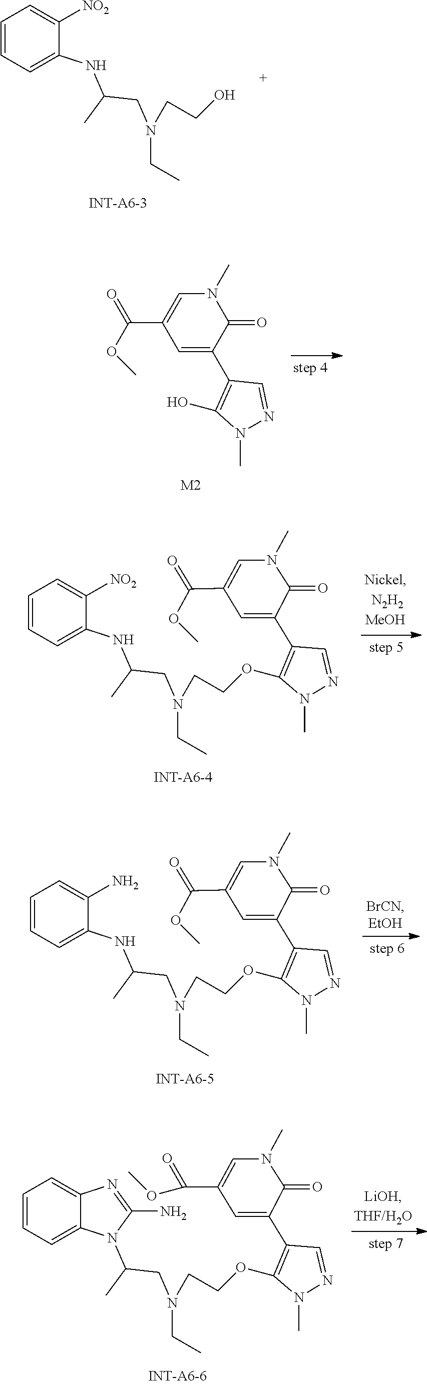 [SMILES: CCN(CCO)CC(C)Nc1ccccc1[N+](=O)[O-] ; COC(=O)c1cc(-c2cnn(C)c2O)c(=O)n(C)c1 ; "M2" ; CCN(CCOc1c(-c2cc(C(=O)OC)cn(C)c2=O)cnn1C)CC(C)Nc1ccccc1[N+](=O)[O-] ; CCN(CCOc1c(-c2cc(C(=O)OC)cn(C)c2=O)cnn1C)CC(C)Nc1ccccc1N ; CCN(CCOc1c(-c2cc(C(=O)OC)cn(C)c2=O)cnn1C)CC(C)n1c(N)nc2ccccc21]

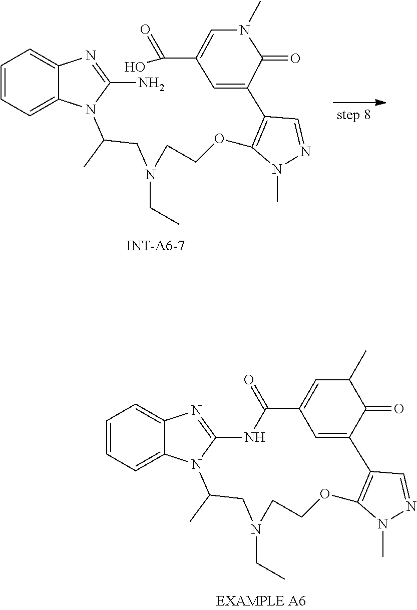 [SMILES: CCN(CCOc1c(-c2cc(C(=O)O)cn(C)c2=O)cnn1C)CC(C)n1c(N)nc2ccccc21 ; CCN1CCOc2c(cnn2C)C2=CC(=CC(C)C2=O)C(=O)Nc2nc3ccccc3n2C(C)C1]

Step 1. Preparation of tert-butyl N-{1-[ethyl(2-hydroxyethyl)amino]propan-2-yl}carbamate (INT-A6-1). To a stirred mixture of ethyl ethanolamine (1 g, 11.21 mmol, 1 equiv) and tert-butyl N-(1-bromopropan-2-yl)carbamate (3.2 g, 13.46 mmol, 1.2 equiv) in acetonitrile (40 mL) was added K₂CO₃ (4.6 g, 33.65 mmol, 3 equiv) at room temperature. The mixture was stirred overnight at 60 °C and cooled to room temperature. The reaction was quenched with water (50 mL) and the mixture was extracted with CH₂Cl₂ (3×100 mL). The combined organic layers were washed with brine (3×100 mL), dried over anhydrous Na₂SO₄, and concentrated under reduced pressure. The residue was purified by Prep-TLC eluted with dichloromethane/methanol (10/1) to give 1 g of tert-butyl N-{1-[ethyl(2-hydroxyethyl)amino]propan-2-yl}carbamate (INT-A6-1) as a colorless oil (36%). ¹H NMR (DMSO-d₆, 400 MHz) δ 1.04 (3H, t), 1.14 (3H, d), 1.46 (9H, s), 2.32-2.47 (2H, m), 2.52-2.70 (4H, m), 3.50-3.63 (2H, m), 3.72-3.80 (1H, m), 4.45 (1H, brs).

Step 2. Preparation of 2-[(2-aminopropyl)(ethyl)amino]ethanol (INT-A6-2). Into a 20 mL vial were added tert-butyl N-{1-[ethyl(2-hydroxyethyl)amino]propan-2-yl}carbamate (1 g, 4.05 mmol, 1 equiv) and HCl in 1,4-dioxane (10 mL) at room temperature. The mixture was stirred for 10 h at room temperature and concentrated under reduced pressure. The residue was treated with saturated NaHCO₃ (aq.) saolution (30 mL) and extracted with ethyl acetate (3×30 mL). The combined organic layers were washed with brine (3×40 mL), dried over anhydrous Na₂SO₄, and concentrated under reduced pressure to give 0.59 g of 2-[(2-aminopropyl)(ethyl)amino]ethanol (INT-A6-2) as a yellow solid. This material was used for the next step without further purification. LCMS: m/z (ESI), [M+H]⁺=146.15

Step 3. Preparation of 2-[ethyl({2-[(2-nitrophenyl)amino]propyl})amino]ethanol (INT-A6-3). To a stirred mixture of 2-[(2-aminopropyl)(ethyl)amino]ethanol (590 mg, −4 mmol, ~1- equiv) and O-fluoronitrobenzene (569 mg, 4.03 mmol, 1 equiv) in acetonitrile (20 mL) was added K₂CO₃ (1115 mg, 8.07 mmol, 2 equiv) at room. The resulting mixture was stirred for 2 h at 60 °C and cooled to room temperature. The reaction was quenched with water (20 mL) and the mixture was extracted with ethyl acetate (3×50 mL). The combined organic layers were washed with brine (3×100 mL), dried over anhydrous Na₂SO₄, and concentrated under reduced pressure. The residue was purified by Prep-TLC eluted with dichloromethane/methanol (12/1) to give 640 mg of 2-[ethyl({2-[(2-nitrophenyl)amino]propyl})amino]ethanol (INT-A6-3) as a yellow solid (59%). LCMS: m/z (ESI), [M+H]⁺=268.10. ¹H NMR (CDCl₃, 400 MHz) δ 1.16 (3H, t), 1.35 (3H, d), 2.34-3.01 (7H, m), 3.64-3.68 (2H, m), 6.68 (1H, t), 6.96 (1H, s), 7.47 (1H, t), 8.18-8.21 (2H, m).

Step 4. Preparation of methyl 5-(5-{2-[ethyl({2-[(2-nitrophenyl)amino]propyl})amino]ethoxy}-1-methylpyrazol-4-yl)-1-methyl-6-oxopyridine-3-carboxylate (INT-A6-4). To a stirred mixture of triphenylphosphine (1766 mg, 6.73 mmol, 3 equiv) and methyl 5-(5-hydroxy-1-methylpyrazol-4-yl)-1-methyl-6-oxopyridine-3-carboxylate (M2, 590 mg, 2.24 mmol, 1 equiv) in tetrahydrofuran (20 mL) was added diisopropyl azodicarboxylate (1361 mg, 6.73 mmol, 3 equiv). The mixture was stirred for 0.5 h at 0 °C under nitrogen atmosphere. To the above mixture was added 2-[ethyl({2-[(2-nitrophenyl)amino]propyl})amino]ethanol (600 mg, 2.24 mmol, 1 equiv) in tetrahydrofuran (2 mL) over 2 min at 0 °C. The mixture was stirred for additional 2 h at 0 °C. The reaction was quenched with water (20 mL) and the mixture was extracted with ethyl acetate (3×50 mL). The combined organic layers were washed with brine (3×100 mL), dried over anhydrous Na₂SO₄, and concentrated under reduced pressure. The residue was purified by Prep-TLC eluted with petroleum ether/ethyl acetate (1/3) to give 400 mg of methyl 5-(5-{2-[ethyl({2-[(2-nitrophenyl)amino]propyl})amino]ethoxy}-1-methylpyrazol-4-yl)-1-methyl-6-oxopyridine-3-carboxylate (INT-A6-4) as a yellow oil (34%). LCMS: m/z (EST), [M+H]=513.20. ¹H NMR (methanol-d₄, 400 MHz) δ 1.09 (3H, t), 1.23-1.31 (3H, m), 2.60-2.75 (3H, m), 2.84-2.95 (1H, m), 2.96-3.03 (1H, m), 3.66 (3H, s), 3.68 (3H, s), 3.80-3.92 (4H, m), 4.01 (2H, t), 6.42-6.62 (1H, m), 6.99 (1H, d), 7.39 (1H, d), 7.91 (1H, s), 8.05 (1H, d), 8.16 (1H, d), 8.35 (1H, d).

Step 5. Preparation of methyl 5-{5-[2-({2-[(2-aminophenyl)amino]propyl}(ethyl)amino)ethoxy]-1-methylpyrazol-4-yl}-1-methyl-6-oxopyridine-3-carboxylate (INT-A6-5). To a stirred mixture of methyl 5-(5-{2-[ethyl({2-[(2-nitrophenyl)amino]propyl})amino]ethoxy}-1-methylpyrazol-4-yl)-1-methyl-6-oxopyridine-3-carboxylate (450 mg, 0.87 mmol, 1 equiv) and Raney-Ni (50 mg) in methanol (5 mL) was added hydrazine hydrate (140 mg, 4.39 mmol, 5 equiv) at 0 °C. The mixture was stirred for 1 h at room temperature under nitrogen atmosphere and filtered. The filter cake was washed with methanol (3×20 mL) and the solution was concentrated under reduced pressure to give 260 mg of methyl 5-{5-[2-({2-[(2-aminophenyl)amino]propyl}(ethyl)amino)ethoxy]-1-methylpyrazol-4-yl}-1-methyl-6-oxopyridine-3-carboxylate (INT-A6-5) as a yellow oil (61%). LCMS: m/z (EST), [M+H]⁺=483.10.

Step 6. Preparation of dimethyl 5-[5-(2-{[2-(2-amino-1,3-benzodiazol-1-yl)propyl](ethyl)amino}ethoxy)-1-methylpyrazol-4-yl]-1-methyl-6-oxopyridine-3-carboxylate (INT-A6-6). A mixture of methyl 5-{5-[2-({2-[(2-aminophenyl)amino]propyl}(ethyl)amino)ethoxy]-1-methylpyrazol-4-yl}-1-methyl-6-oxopyridine-3-carboxylate (260 mg, 0.53 mmol, 1 equiv) and BrCN (114 mg, 1.07 mmol, 2 equiv) in ethanol (5 mL) was stirred for 1 h at room temperature under nitrogen atmosphere and concentrated under reduced pressure. The residue was purified by Prep-TLC eluted with dichloromethane/methanol (20/1) to give 250 mg of dimethyl 5-[5-(2-{[2-(2-amino-1,3-benzodiazol-1-yl)propyl](ethyl)amino}ethoxy)-1-methylpyrazol-4-yl]-1-methyl-6-oxopyridine-3-carboxylate (INT-A6-6) as a yellow solid (91%). LCMS: m/z (ESI), [M+H]+=508.40.

Step 7. Preparation of 5-[5-(2-{[2-(2-amino-1,3-benzodiazol-1-yl)propyl](ethyl)amino}ethoxy)-1-methylpyrazol-4-yl]-1-methyl-6-oxopyridine-3-carboxylic acid (INT-A6-7). To a stirred solution of methyl 5-[5-(2-{[2-(2-amino-1,3-benzodiazol-1-yl)propyl](ethyl)amino}ethoxy)-1-methylpyrazol-4-yl]-1-methyl-6-oxopyridine-3-carboxylate (240 mg, 0.47 mmol, 1 equiv) in tetrahydrofuran (6 mL) was added LiOH·H₂O (33 mg, 1.41 mmol, 3 equiv) in H₂O (1.5 mL). The resulting mixture was stirred for 2 h at room temperature and concentrated under reduced pressure. The residue was purified by reverse flash chromatography with C18 silica gel column using water and acetonitrile as mobile phase. Fractions containing the desired compound were evaporated to dryness to give 200 mg of [5-(2-{[2-(2-amino-1,3-benzodiazol-1-yl)propyl](ethyl)amino}ethoxy)-1-methylpyrazol-4-yl]-1-methyl-6-oxopyridine-3-carboxylic acid (INT-A6-7) as a yellow solid (85%). LCMS: m/z (EST), [M+H]⁺=494.40.

Step 8. Preparation of 10-ethyl-5,12,26-trimethyl-7-oxa-4,5,10,13,20,22,26-heptaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (EXAMPLE A6). To a stirred mixture of 5-[5-(2-{[2-(2-amino-1,3-benzodiazol-1-yl)propyl](ethyl)amino}ethoxy)-1-methylpyrazol-4-yl]-1-methyl-6-oxopyridine-3-carboxylic acid (110 mg, 0.22 mmol, 1 equiv) and N,N-diisopropylethylamine (115 mg, 0.89 mmol, 4 equiv) in 1,4-dioxane (6 mL) was added N,N,N,N-tetramethyl-O-(7-azabenzotriazol-1-yl)uroniumhexafluorophospate (169 mg, 0.44 mmol, 2 equiv) at room temperature under air atmosphere. The resulting mixture was stirred for 2 h at 60 °C, cooled to room temperature, and concentrated under reduced pressure. The residue was purified by Prep-TLC eluted with dichloromethane/methanol (12/1) and further purified by Prep-HPLC with XBridge Shield RP18 OBD column using water containing 0.10% NH₄HCO₃ and 0.1% NH₃·H₂O and acetonitrile. Fractions containing the desired compound were evaporated to dryness to give 28.8 mg of 10-ethyl-5,12,26-trimethyl-7-oxa-4,5,10,13,20,22,26-heptaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (EXAMPLE A6) as a white solid (27%). LCMS: m/z (ESI), [M+H]⁺=476.30. ¹H NMR (DMSO-d₆, 400 MHz) δ 1.07 (3H, t), 1.62 (3H, d), 2.53-2.65 (2H, m), 2.75-2.82 (2H, m), 3.33-3.58 (2H, m), 3.62 (3H, s), 3.75 (3H, s), 4.09 (1H, s), 4.28-4.37 (1H, m), 5.15 (1H, s), 7.14-7.29 (2H, m), 7.47-7.60 (1H, m), 7.60-7.71 (1H, m), 8.17 (1H, s), 8.27 (1H, d), 8.88 (1H, d), 12.54 (1H, s).

### Example A7

10-Ethyl-5,12,12,26-tetramethyl-7-oxa-4,5,10,13,20,22,26-heptaazapentacyclo[22.3.1.0\hat{ }{2,6}.0\hat{ }{13,21}.0\hat{ }{14,19}]octacosa-1(28),2(6),3,14,16,18,20,24-octaene-23,27-dione

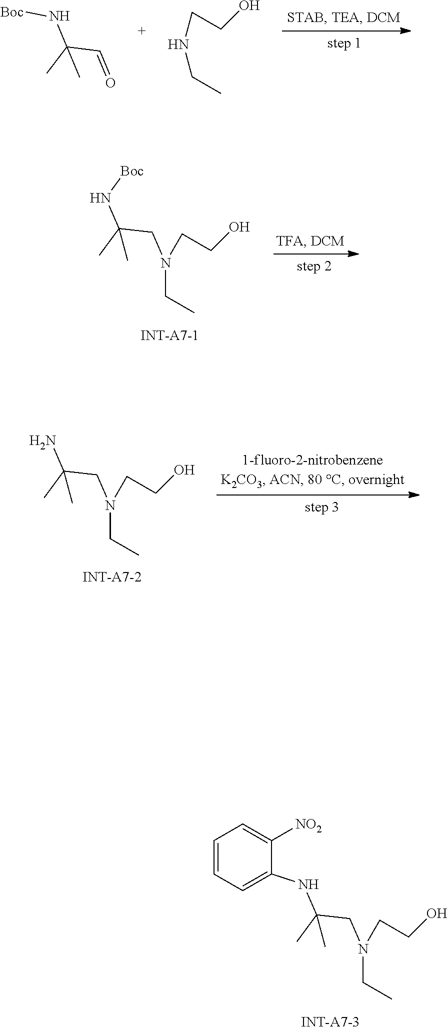

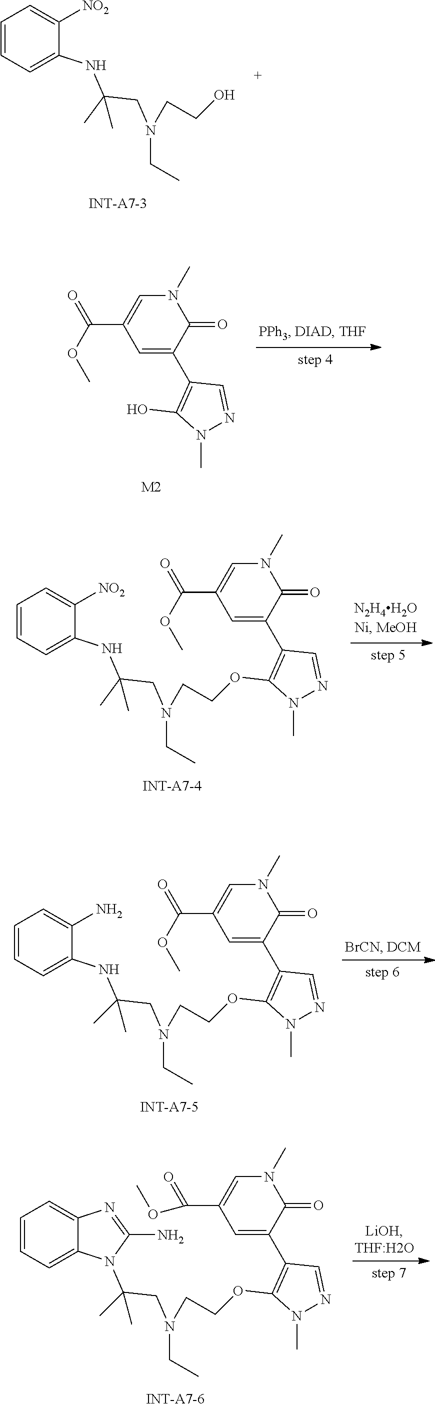 [SMILES: CCN(CCO)CC(C)(C)Nc1ccccc1[N+](=O)[O-] ; COC(=O)c1cc(-c2cnn(C)c2O)c(=O)n(C)c1 ; "M2" ; CCN(CCOc1c(-c2cc(C(=O)OC)cn(C)c2=O)cnn1C)CC(C)(C)Nc1ccccc1[N+](=O)[O-] ; CCN(CCOc1c(-c2cc(C(=O)OC)cn(C)c2=O)cnn1C)CC(C)(C)Nc1ccccc1N ; CCN(CCOc1c(-c2cc(C(=O)OC)cn(C)c2=O)cnn1C)CC(C)(C)n1c(N)nc2ccccc21]

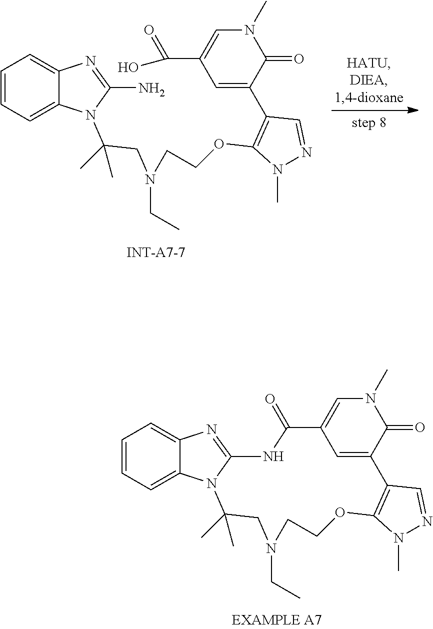 [SMILES: CCN(CCOc1c(-c2cc(C(=O)O)cn(C)c2=O)cnn1C)CC(C)(C)n1c(N)nc2ccccc21 ; CCN1CCOc2c(cnn2C)-c2cc(cn(C)c2=O)C(=O)Nc2nc3ccccc3n2C(C)(C)C1]

Step 1. Preparation of tert-butyl N-{1-[ethyl(2-hydroxyethyl)amino]-2-methylpropan-2-yl}carbamate (INT-A7-1). A mixture of tert-butyl N-(2-methyl-1-oxopropan-2-yl)carbamate (5 g, 26.70 mmol, 1 equiv), triethylamine (8 g, 80.11 mmol, 3 equiv) and ethyl ethanolamine (2.4 g, 26.70 mmol, 1.00 equiv) in dichloromethane (50 mL) was stirred for 1 h at room temperature under nitrogen atmosphere. Then NaBH(OAc)₃ (17 g, 80.11 mmol, 3 equiv) was added and the mixture was stirred overnight at room temperature under nitrogen atmosphere. The reaction was quenched with water (300 mL) and the mixture was extracted with ethyl acetate (3×200 mL). The combined organic layers were washed with brine (3×300 mL), dried over anhydrous Na₂SO₄, and concentrated under reduced pressure. The residue was purified by C18 column using water and acetonitrile as mobile phase. Fractions containing the desired compound were evaporated to dryness to give 5 g of tert-butyl N-{1-[ethyl(2-hydroxyethyl)amino]-2-methylpropan-2-yl}carbamate (INT-A7-1) as a colorless oil (72%). LCMS: m/z (ESI), [M+H]⁺=261.10. ¹H NMR (DMSO-d₆, 400 MHz) δ 0.93 (3H, t), 1.13-1.14 (6H, s), 1.36-1.39 (9H, d), 2.49-2.53 (3H, m), 2.53-2.56 (3H, m), 3.42-3.43 (2H, m), 4.31 (1H, s), 6.25 (1H, s).

Step 2. Preparation of 2-((2-amino-2-methylpropyl)(ethyl)amino)ethan-1-ol (INT-A7-2).

A mixture of tert-butyl N-{1-[ethyl(2-hydroxyethyl)amino]-2-methylpropan-2-yl}carbamate (2 g, 3.84 mmol, 1 equiv) in trifluoroaceticn acid (5 mL) and dichloromethane (15 mL) was stirred for 2 h at room temperature under nitrogen atmosphere. The resulting mixture was concentrated under reduced pressure. The residue was treated with saturated NaHCO₃ (aq.) solution (100 mL) and extracted with dichloromethane (3×100 mL). The combined organic layers were washed with brine (3×200 mL), dried over anhydrous Na₂SO₄, and concentrated under reduced pressure to give 1.23 g of 2-((2-amino-2-methylpropyl)(ethyl)amino)ethan-1-ol (INT-A7-2) as a colorless oil. This material was used for the next step without further purification. LCMS: m/z (ESI), [M+H]⁺=161.10.

Step 3. Preparation of 2-[ethyl({2-methyl-2-[(2-nitrophenyl)amino]propyl})amino]ethanol (INT-A7-3). A mixture of 2-[(2-amino-2-methylpropyl)(ethyl)amino]ethanol (2 g, 12.48 mmol, 1 equiv), K₂CO₃ (5 g, 37.44 mmol, 3 equiv) and O-fluoronitrobenzene (1.7 g, 12.48 mmol, 1 equiv) in acetonitrile (20 mL) was stirred at 80 °C overnight under nitrogen atmosphere. The mixture was cooled to room temperature. The reaction was quenched with water (100 mL) and the mixture was extracted with dichloromethane (3×100 mL). The combined organic layers were washed with brine (3×100 mL), dried over anhydrous Na₂SO₄, and concentrated under reduced pressure. The residue was purified by prep-TLC eluted with dichloromethane/methanol (20/1) to give 1.2 g of 2-[ethyl({2-methyl-2-[(2-nitrophenyl)amino]propyl})amino]ethanol (INT-A7-3) as a yellow oil (34%). LCMS: m/z (ESI), [M+H]⁺=282.05. ¹H NMR (DMSO-d₆, 400 MHz) δ 0.97 (3H, t), 1.41 (6H, s), 2.62-2.66 (3H, m), 2.69 (2H, s), 3.17 (1H, d), 3.44-3.50 (2H, m), 4.37 (1H, t), 6.62-6.68 (1H, m), 7.26-7.33 (1H, m), 7.44-7.52 (1H, m), 8.04-8.10 (1H, m), 8.62-8.68 (1H, s).

Step 4. Preparation of methyl 5-(5-(2-(ethyl(2-methyl-2-((2-nitrophenyl)amino)propyl)amino)ethoxy)-1-methyl-1H-pyrazol-4-yl)-1-methyl-6-oxo-1,6-dihydropyridine-3-carboxylate (INT-A7-4). To a stirred mixture of methyl 5-(5-hydroxy-1-methylpyrazol-4-yl)-1-methyl-6-oxopyridine-3-carboxylate (M2, 281 mg, 1.06 mmol, 1 equiv), triphenylphosphine (839 mg, 3.19 mmol, 3 equiv) in tetrahydrofuran (10 mL) were added diisopropyl azodicarboxylate (646 mg, 3.19 mmol, 3 equiv) and 2-[ethyl({2-methyl-2-[(2-nitrophenyl)amino]propyl})amino]ethanol (300 mg, 1.06 mmol, 1 equiv) at 0 °C under nitrogen atmosphere. The mixture was stirred for 1 h, treated with water (100 mL), and extracted with dichloromethane (2×100 mL). The combined organic layers were washed with brine (2×100 mL), dried over anhydrous Na₂SO₄, and concentrated under reduced pressure. The residue was purified by prep-TLC eluted with dichloromethane/methanol (12/1) to give 230 mg of methyl 5-(5-(2-(ethyl(2-methyl-2-((2-nitrophenyl)amino)propyl)amino)ethoxy)-1-methyl-1H-pyrazol-4-yl)-1-methyl-6-oxo-1,6-dihydropyridine-3-carboxylate (INT-A7-4) as a yellow solid (41%). LCMS: m/z (ESI), [M+H]⁺=527.20. ¹H NMR (DMSO-d₆, 400 MHz) δ 0.98 (3H, t), 1.39 (6H, s), 2.67-2.69 (2H, m), 2.74 (2H, s), 2.98 (2H, t), 3.56 (3H, s), 3.65 (3H, s), 3.77 (3H, s), 4.00 (2H, t), 5.77 (1H, s), 6.62-6.67 (1H, m), 7.23-7.28 (1H, d), 7.41-7.48 (1H, m), 7.90-7.93 (1H, s), 8.00-8.04 (2H, m), 8.40 (1H, d), 8.58 (1H, s)

Step 5. Preparation of methyl 5-{5-[2-({2-[(2-aminophenyl)amino]-2-methylpropyl}(ethyl)amino)ethoxy]-1-methylpyrazol-4-yl}-1-methyl-6-oxopyridine-3-carboxylate (INT-A7-5). To a stirred mixture of methyl 5-(5-{2-[ethyl({2-methyl-2-[(2-nitrophenyl)amino]propyl})amino]ethoxy}-1-methylpyrazol-4-yl)-1-methyl-6-oxopyridine-3-carboxylate (230 mg, 0.44 mmol, 1 equiv), and Raney-Ni (50 mg) in methanol (2 mL) was added NH₂NH₂H₂O (22 mg, 0.44 mmol, 1 equiv) at 0 °C under nitrogen atmosphere. The mixture was stirred for 1 h at 0 °C. The reaction was quenched with water (50 mL) and the mixture was extracted with ethyl acetate (3×50 mL). The combined organic layers were washed with brine (3×50 mL), dried over anhydrous Na₂SO₄, and concentrated under reduced pressure. The residue was purified by prep-TLC eluted with dichloromethane/methanol (10/1) to give 110 mg of methyl 5-{5-[2-({2-[(2-aminophenyl)amino]-2-methylpropyl}(ethyl)amino)ethoxy]-1-methylpyrazol-4-yl}-1-methyl-6-oxopyridine-3-carboxylate (INT-A7-5) as a yellow solid (51%). LCMS: m/z (ESI), [M+H]⁺=497.40. ¹H NMR (DMSO-d₆, 400 MHz) δ 0.97 (3H, t), 1.14 (6H, s), 2.62-2.69 (2H, m), 2.94 (2H, t), 3.18 (2H, d), 3.58 (3H, s), 3.67 (4H, s), 3.80 (3H, s), 4.00 (2H, t), 4.38 (5H, s), 4.47 (2H, s), 6.35-6.39 (2H, m), 6.47-6.54 (4H, m), 6.72 (1H, d), 7.96 (1H, s), 8.11 (1H, d), 8.46 (1H, d).

Step 6. Preparation of methyl 5-(5-(2-((2-(2-amino-1H-benzo[d]imidazol-1-yl)-2-methylpropyl)(ethyl)amino)ethoxy)-1-methyl-1H-pyrazol-4-yl)-1-methyl-6-oxo-1,6-dihydropyridine-3-carboxylate (INT-A7-6). A mixture of methyl 5-{5-[2-({2-[(2-aminophenyl)amino]-2-methylpropyl}(ethyl)amino)ethoxy]-1-methylpyrazol-4-yl}-1-methyl-6-oxopyridine-3-carboxylate (200 mg, 0.40 mmol, 1 equiv) and BrCN (51 mg, 0.48 mmol, 1.2 equiv) in dichloromethane (5 mL) was stirred for 2 h at room temperature under nitrogen atmosphere and concentrated under vacuum. The residue was purified by prep-TLC eluted with dichloromethane/methanol (12/1) to give 120 mg of methyl 5-(5-(2-((2-(2-amino-1H-benzo[d]imidazol-1-yl)-2-methylpropyl)(ethyl)amino)ethoxy)-1-methyl-1H-pyrazol-4-yl)-1-methyl-6-oxo-1,6-dihydropyridine-3-carboxylate (INT-A7-6) as a yellow solid (61%). LCMS: m/z (ESI), [M+H]⁺=522.30. ¹H NMR (DMSO-d₆, 400 MHz) δ 0.82 (3H, t), 1.81 (6H, s), 2.44-2.48 (3H, m), 2.79 (2H, t), 3.02 (2H, s), 3.58 (6H, d), 3.78-3.82 (5H, m), 6.88 (1H, t), 6.99 (1H, t), 7.19 (1H, s), 7.55 (1H, d), 7.95 (1H, s), 8.05 (1H, d), 8.47 (1H, d)

Step 7. Preparation of 5-(5-(2-((2-(2-amino-1H-benzo[d]imidazol-1-yl)-2-methylpropyl)(ethyl)amino)ethoxy)-1-methyl-1H-pyrazol-4-yl)-1-methyl-6-oxo-1,6-dihydropyridine-3-carboxylic acid (INT-A7-7). A mixture of methyl 5-[5-(2-{[2-(2-amino-1,3-benzodiazol-1-yl)-2-methylpropyl](ethyl)amino}ethoxy)-1-methylpyrazol-4-yl]-1-methyl-6-oxopyridine-3-carboxylate (120 mg, 0.23 mmol, 1 equiv) and LiOH·H₂O (39 mg, 0.92 mmol, 4 equiv) in tetrahydrofuran/H₂O (2 mL/0.5 mL) was stirred for 2 h at 40 °C under nitrogen atmosphere. The mixture was cooled to room temperature and concentrated under reduced pressure. The residue was purified by C18 column using water and acetonitrile as mobile phase. Fractions containing the desired compound were evaporated to dryness to give 65 mg of 5-(5-(2-((2-(2-amino-1H-benzo[d]imidazol-1-yl)-2-methylpropyl)(ethyl)amino)ethoxy)-1-methyl-1H-pyrazol-4-yl)-1-methyl-6-oxo-1,6-dihydropyridine-3-carboxylic acid (INT-A7-7) as a white solid (56%). LCMS: m/z (ESI), [M+H]⁺=508.15. ¹H NMR (DMSO-d₆, 400 MHz) δ 0.77 (3H, t), 1.79 (6H, s), 2.35-2.42 (3H, m), 2.67-2.69 (1H, m), 2.81 (2H, t), 2.99 (2H, s), 3.48 (3H, s), 3.56 (3H, s), 3.82 (2H, t), 6.30 (2H, s), 6.78 (1H, t), 6.86-6.93 (1H, m), 7.06-7.11 (1H, m), 7.46 (1H, d), 7.86 (1H, s), 7.96 (1H, s), 8.17 (1H, d)

Step 8. Preparation of 10-ethyl-5,12,12,26-tetramethyl-7-oxa-4,5,10,13,20,22,26-heptaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (EXAMPLE A7). A mixture of 5-[5-(2-{[2-(2-amino-1,3-benzodiazol-1-yl)-2-methylpropyl](ethyl)amino}ethoxy)-1-methylpyrazol-4-yl]-1-methyl-6-oxopyridine-3-carboxylic acid (65 mg, 0.13 mmol, 1 equiv), N,N,N,N-tetramethyl-O-(7-azabenzotriazol-1-yl)uronium hexafluorophospate (97 mg, 0.25 mmol, 2 equiv) and N,N-diisopropylethylamine (33 mg, 0.25 mmol, 2 equiv) in 1,4-dioxane (2 mL) was stirred for 2 h at 60 °C under nitrogen atmosphere. The mixture was cooled to room temperature. The reaction was quenched with water (50 mL) and the mixture was extracted with dichloromethane (3×50 mL). The combined organic layers were washed with brine (3×50 mL), dried over anhydrous Na₂SO₄, and concentrated under reduced pressure. The residue was purified by prep-HPLC with XBridge Shield RP18 OBD column using water containing 10 mmol/L NH₄HCO₃ and 0.1% NH₃—H₂O and acetonitrile as mobile phase. Fractions containing the desired compound were evaporated to dryness to give 13.6 mg of 10-ethyl-5,12,12,26-tetramethyl-7-oxa-4,5,10,13,20,22,26-heptaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (EXAMPLE A7) as a white solid (22%). LCMS: m/z (EST), [M+H]⁺=490.20. ¹H NMR (DMSO-d, 400 MHz) δ 0.99 (3H, t), 1.94 (6H, s), 2.68 (2H, d), 3.08-3.22 (2H, m), 3.60-3.64 (5H, m), 3.73 (3H, s), 4.28 (2H, d), 7.17 (2H, t), 7.56-7.63 (1H, m), 7.83 (1H, d), 8.13 (1H, d), 8.29 (1H, d), 8.67 (1H, d), 12.93 (1H, s).

### Example A8

### 15,21-Dimethyl-23-oxa-2,9,11,15,20,21,26-heptaazahexacyclo[24.4.1.1\hat{ }{13,17}.0\hat{ }{2,10}.0\hat{ }{3,8}.0\hat{ }{18,22}]dotriaconta-3,5,7,9,13,17(32),18(22),19-octaene-12,16-dione

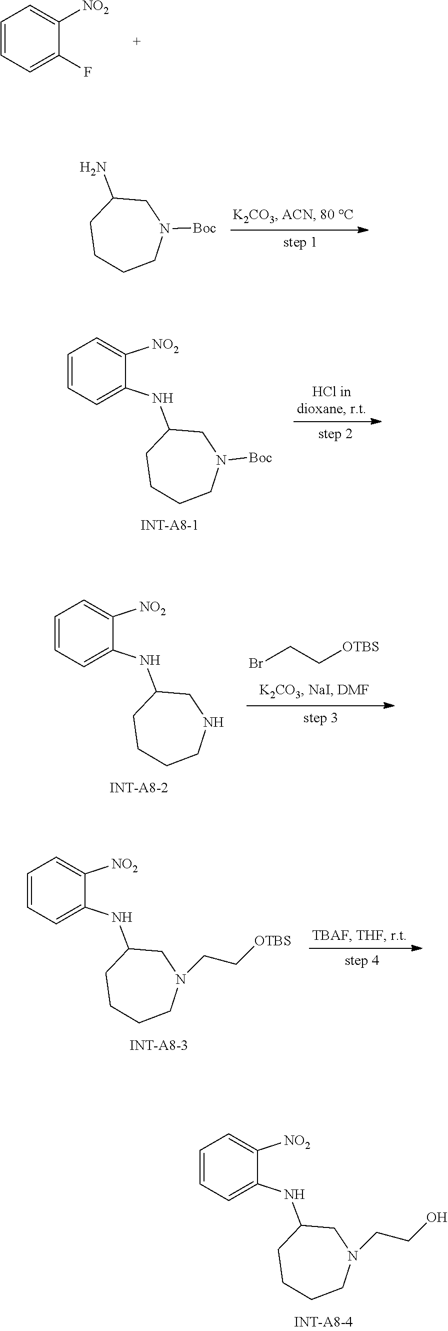

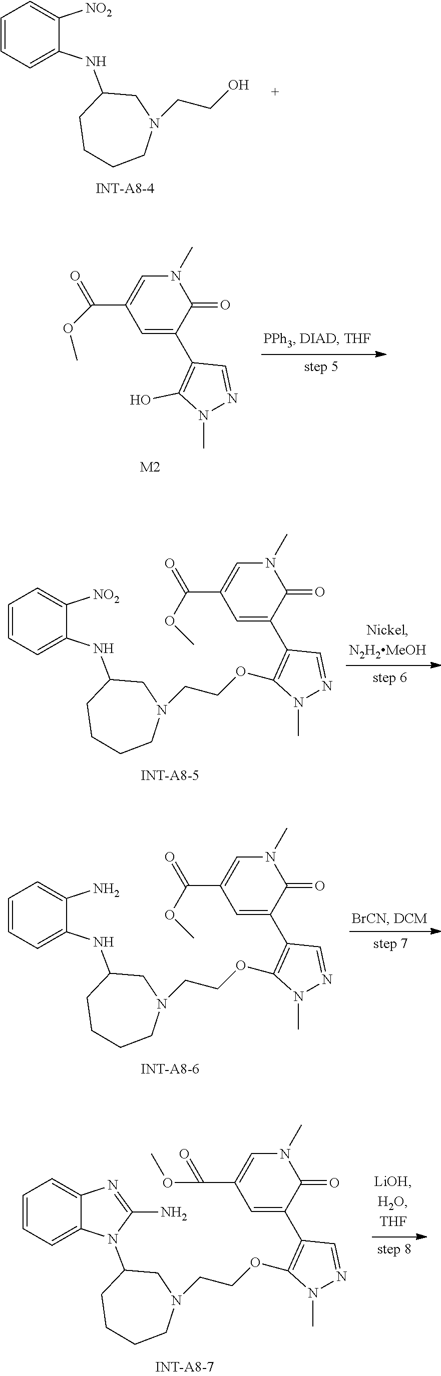 [SMILES: O=[N+]([O-])c1ccccc1NC1CCCCN(CCO)C1 ; COC(=O)c1cc(-c2cnn(C)c2O)c(=O)n(C)c1 ; "M2" ; COC(=O)c1cc(-c2cnn(C)c2OCCN2CCCCC(Nc3ccccc3[N+](=O)[O-])C2)c(=O)n(C)c1 ; COC(=O)c1cc(-c2cnn(C)c2OCCN2CCCCC(Nc3ccccc3N)C2)c(=O)n(C)c1 ; COC(=O)c1cc(-c2cnn(C)c2OCCN2CCCCC(n3c(N)nc4ccccc43)C2)c(=O)n(C)c1]

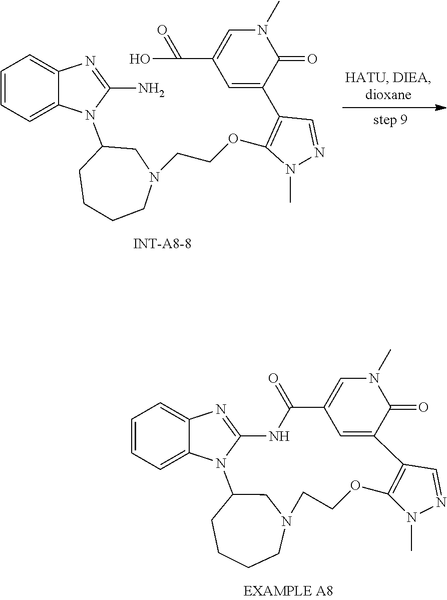 [SMILES: Cn1ncc(-c2cc(C(=O)O)cn(C)c2=O)c1OCCN1CCCCC(n2c(N)nc3ccccc32)C1 ; Cn1ncc2c1OCCN1CCCCC(C1)n1c(nc3ccccc31)NC(=O)c1cc-2c(=O)n(C)c1]

Step 1. Preparation of tert-butyl 3-((2-nitrophenyl)amino)azepane-1-carboxylate (INT-A8-1). To a stirred mixture of O-fluoronitrobenzene (5 g, 35.43 mmol, 1 equiv) and tert-butyl 3-aminoazepane-1-carboxylate (7.9 g, 37.20 mmol, 1.05 equiv) in acetonitrile (80 mL) was added K₂CO₃ (9.7 g, 70.87 mmol, 2 equiv). The mixture was stirred for 3 h at 80 °C under nitrogen atmosphere and cooled to room temperature. The reaction was quenched with water (100 mL) and the mixture was extracted with CH₂Cl₂ (3×100 mL). The combined organic layers were washed with brine (3×100 mL), dried over anhydrous Na₂SO₄, and concentrated under reduced pressure. The residue was purified on a silica gel column and eluted with petroleum ether/ethyl acetate (10:1) to give 10.6 g of tert-butyl 3-[(2-nitrophenyl)amino]azepane-1-carboxylate (INT-A8-1) as a brown oil (89%). LCMS: m/z (ESI), [M+H]⁺=336.15. ¹H NMR (DMSO-d₆, 400 MHz) δ 1.37 (9H, d), 1.47-2.03 (6H, m), 3.34-3.49 (3H, m), 3.51-3.62 (1H, m), 3.93 (1H, s), 6.61-6.75 (1H, m), 7.20 (1H, m), 7.47-7.55 (1H, m), 7.97-8.22 (2H, m).

Step 2. Preparation of N-(2-nitrophenyl)azepan-3-amine (INT-A8-2). A mixture of tert-butyl 3-[(2-nitrophenyl)amino]azepane-1-carboxylate (12 g, 35.77 mmol, 1 equiv) and HCl in 1,4-dioxane (100 mL) was stirred for 1.5 h at room temperature. The mixture was concentrated under reduced pressure and treated with CH₂Cl₂ (60 mL). The mixture was treated with saturated NaHCO₃ (aq.) solution (50 mL) and extracted with CH₂Cl₂ (3×100 mL). The combined organic layers were dried over anhydrous Na₂SO₄ and concentrated under reduced pressure to give 8 g of N-(2-nitrophenyl)azepan-3-amine (INT-A8-2) as a yellow oil. This material was used for the next reaction without further purification. LCMS: m/z (ESI), [M+H]⁺=236.20.

Step 3. Preparation of 1-(2-((tert-butyldimethylsilyl)oxy)ethyl)-N-(2-nitrophenyl)azepan-3-amine (INT-A8-3). To a stirred mixture of N-(2-nitrophenyl)azepan-3-amine (1 g, 4.25 mmol, 1 equiv) and (2-bromoethoxy)(tert-butyl)dimethylsilane (2.03 g, 8.50 mmol, 2 equiv) in N,N-dimethylformamide (15 mL) were added K₂CO₃ (1.17 g, 8.50 mmol, 2 equiv) and KI (1.41 g, 8.50 mmol, 2 equiv). The resulting mixture was stirred for 2 h at 60 °C under nitrogen atmosphere. The mixture was cooled to room temperature. The reaction was quenched with water (100 mL) and the mixture was extracted with CH₂Cl₂ (3×100 mL). The combined organic layers were washed with brine (3×100 mL), dried over anhydrous Na₂SO₄, and concentrated under reduced pressure. The residue was purified on a silica gel column and eluted with ethyl acetate/petroleum ether (1/10) to give 819 mg of 1-{2-[(tert-butyldimethylsilyl)oxy]ethyl}-N-(2-nitrophenyl)azepan-3-amine (INT-A8-3) as a yellow oil (48%). LCMS: m/z (EST), [M+H]⁺=394.20. ¹H NMR (DMSO-d₆, 400 MHz) δ −0.02 (6H, d), 0.81 (9H, s), 1.35-1.65 (3H, m), 1.65-1.85 (3H, m), 2.57-2.80 (3H, m), 2.81-3.03 (3H, m), 3.61-3.82 (2H, m), 3.93 (1H, s), 6.64-6.66 (1H, m), 7.04 (1H, d), 7.45-7.55 (1H, m), 8.02-8.09 (1H, m), 8.73 (1H, d).

Step 4. Preparation of 2-(3-((2-nitrophenyl)amino)azepan-1-yl)ethan-1-ol (INT-A8-4). To a solution of 1-{2-[(tert-butyldimethylsilyl)oxy]ethyl}-N-(2-nitrophenyl)azepan-3-amine (447 mg, 1.13 mmol, 1 equiv) in tetrahydrofuran (10 mL) was added tetrabutylammonium fluoride (593 mg, 2.272 mmol, 2 equiv). The mixture was stirred for 2 h at room temperature. The reaction was quenched with water (50 mL) and the mixture was extracted with ethyl acetate (3×50 mL). The combined organic layers were washed with brine (3×100 mL), dried over anhydrous Na₂SO₄, and concentrated under reduced pressure to give 447 mg of 2-{3-[(2-nitrophenyl)amino]azepan-1-yl}ethanol (INT-A8-4) as a yellow oil. This material was used for the next reaction without further purification. LCMS: m/z (ESI), [M+H]⁺=280.05.

Step 5. Preparation of methyl 1-methyl-5-(1-methyl-5-(2-(3-((2-nitrophenyl)amino)azepan-1-yl)ethoxy)-1H-pyrazol-4-yl)-6-oxo-1,6-dihydropyridine-3-carboxylate (INT-A8-5). To a stirred mixture of methyl 5-(5-hydroxy-1-methylpyrazol-4-yl)-1-methyl-6-oxopyridine-3-carboxylate (M2, 207 mg, 0.78 mmol, 1.1 equiv) and triphenylphosphine (563 mg, 2.14 mmol, 3 equiv) in tetrahydrofuran (10 mL) were added diisopropyl azodicarboxylate (434 mg, 2.14 mmol, 3 equiv) and 2-{3-[(2-nitrophenyl)amino]azepan-1-yl}ethanol (200 mg, 0.71 mmol, 1 equiv) at 0 °C under nitrogen atmosphere. The resulting mixture was stirred overnight at room temperature. The reaction was quenched with water (50 mL) and the mixture was extracted with ethyl acetate (3×50 mL). The combined organic layers were washed with brine (3×100 mL), dried over anhydrous Na₂SO₄, and concentrated under reduced pressure. The residue was purified by Prep-TLC eluted with ethyl acetate to give 301 mg of methyl 1-methyl-5-[1-methyl-5-(2-{3-[(2-nitrophenyl)amino]azepan-1-yl}ethoxy)pyrazol-4-yl]-6-oxopyridine-3-carboxylate (INT-A8-5) as an orange solid (80%). LCMS: m/z (ESI), [M+H]⁺=525.30 ¹H NMR (DMSO-d₆, 400 MHz) δ 1.22-1.63 (7H, m), 2.45-2.68 (1H, m), 2.79-2.89 (2H, m), 2.95-3.05 (3H, m), 3.55 (3H, s), 3.68 (3H, s), 3.73 (3H, s), 4.05-4.10 (2H, m), 6.60-6.70 (1H, m), 6.95-7.09 (1H, m), 7.40-7.53 (1H, m), 7.94 (1H, s), 7.99-8.05 (2H, m), 8.32 (1H, d), 8.61 (1H, d)

Step 6. Preparation of methyl 5-(5-(2-(3-((2-aminophenyl)amino)azepan-1-yl)ethoxy)-1-methyl-1H-pyrazol-4-yl)-1-methyl-6-oxo-1,6-dihydropyridine-3-carboxylate (INT-A8-6). To a stirred mixture of Raney nickel (100 mg) and methyl 1-methyl-5-[1-methyl-5-(2-{3-[(2-nitrophenyl)amino]azepan-1-yl}ethoxy)pyrazol-4-yl]-6-oxopyridine-3-carboxylate (300 mg, 0.54 mmol, 1 equiv) in methanol (10 mL) was added NH₂NH₂H₂O (84 mg, 0.28 mmol, 3 equiv) at 0 °C. The mixture was stirred for 2 h at room temperature and filtered. The filter cake was washed with methanol (3×5 mL) and the solution was concentrated under reduced pressure. The residue was purified by Prep-TLC and eluted with dichloromethane/methanol (10/1) to give 89 mg of methyl 5-[5-(2-{3-[(2-aminophenyl)amino]azepan-1-yl}ethoxy)-1-methylpyrazol-4-yl]-1-methyl-6-oxopyridine-3-carboxylate (INT-A8-6) as a yellow oil (44%). LCMS: m/z (ESI), [M+H]⁺=495.30

Step 7. Preparation of methyl 5-(5-(2-(3-(2-amino-1H-benzo[d]imidazol-1-yl)azepan-1-yl)ethoxy)-1-methyl-1H-pyrazol-4-yl)-1-methyl-6-oxo-1,6-dihydropyridine-3-carboxylate (INT-A8-7). A mixture of methyl 5-[5-(2-{3-[(2-aminophenyl)amino]azepan-1-yl}ethoxy)-1-methylpyrazol-4-yl]-1-methyl-6-oxopyridine-3-carboxylate (89 mg, 0.18 mmol, 1 equiv) and BrCN (20 mg, 0.19 mmol, 1.10 equiv) in CH₂Cl₂ (5 mL) was stirred for 5 h at room temperature and concentrated under reduced pressure. The residue was purified by Prep-TLC and eluted with dichloromethane/methanol (10/1) to give 21 mg of methyl 5-(5-{2-[3-(2-amino-1,3-benzodiazol-1-yl)azepan-1-yl]ethoxy}-1-methylpyrazol-4-yl)-1-methyl-6-oxopyridine-3-carboxylate (INT-A8-7) as a white solid (22%). LCMS: m/z (ESI), [M+H]⁺=520.25

Step 8. Preparation of 5-(5-(2-(3-(2-amino-1H-benzo[d]imidazol-1-yl)azepan-1-yl)ethoxy)-1-methyl-1H-pyrazol-4-yl)-1-methyl-6-oxo-1,6-dihydropyridine-3-carboxylic acid (INT-A8-8). To a stirred mixture of methyl 5-(5-{2-[3-(2-amino-1,3-benzodiazol-1-yl)azepan-1-yl]ethoxy}-1-methylpyrazol-4-yl)-1-methyl-6-oxopyridine-3-carboxylate (60 mg, 0.11 mmol, 1 equiv) in tetrahydrofuran (4 mL) and H₂O (1 mL) was added LiOH·H₂O (8 mg, 0.34 mmol, 3 equiv). The resulting mixture was stirred for 8 h at room temperature and concentrated under reduced pressure. The mixture was purified by reverse flash chromatography with C18 silica gel column using water and acetonitrile as mobile phase. Fractions containing the desired compound were evaporated to dryness to give 16.1 mg of 5-(5-{2-[3-(2-amino-1,3-benzodiazol-1-yl)azepan-1-yl]ethoxy}-1-methylpyrazol-4-yl)-1-methyl-6-oxopyridine-3-carboxylic acid (INT-A8-8) as white solid (42%). LCMS: m/z (ESI), [M+H]⁺=506.20

Step 9. Preparation of 15,21-dimethyl-23-oxa-2,9,11,15,20,21,26-heptaazahexacyclo[24.4.1.1\hat{ }{13,17}.0\hat{ }{2,10}.0\hat{ }{3,8}.0\hat{ }{18,22}]dotriaconta-3,5,7,9,13,17(32),18(22),19-octaene-12,16-dione (EXAMPLE A8). To a stirred mixture of 5-(5-{2-[3-(2-amino-1,3-benzodiazol-1-yl)azepan-1-yl]ethoxy}-1-methylpyrazol-4-yl)-1-methyl-6-oxopyridine-3-carboxylic acid (15 mg, 0.03 mmol, 1 equiv) and N,N,N,N-tetramethyl-O-(7-azabenzotriazol-1-yl)uroniumhexafluorophospate (33 mg, 0.09 mmol, 3 equiv) in 1,4-dioxane (2 mL) was added N,N-diisopropylethylamine (11 mg, 0.09 mmol, 3 equiv). The resulting mixture was stirred for 3 h at room temperature and concentrated. The residue was purified by Prep-HPLC with XBridge Shield RP18 OBD column using water containing 0.1% NH₄HCO₃ and acetonitrile as mobile phase. Fractions containing the desired compound were evaporated to dryness to give 4 mg of 15,21-dimethyl-23-oxa-2,9,11,15,20,21,26-heptaazahexacyclo[24.4.1.1\hat{ }{13,17}.0\hat{ }{2,10}.0\hat{ }{3,8}.0\hat{ }{18,22}]dotriaconta-3,5,7,9,13,17(32),18(22),19-octaene-12,16-dione (EXAMPLE A8) as a white solid (27%). LCMS: m/z (ESI), [M+H]⁺=488.15. ¹H NMR (DMSO-d₆, 400 MHz) δ 1.65-2.03 (5H, m), 2.54-2.72 (1H, m), 2.72-3.03 (5H, m), 3.08-3.22 (1H, m), 3.63 (3H, s), 3.73 (3H, s), 3.80-3.94 (1H, m), 4.34-4.60 (1H, m), 5.14 (1H, s), 7.16-7.26 (2H, m), 7.50-7.57 (1H, m), 7.62 (1H, d), 8.27 (1H, d), 8.31 (1H, s), 8.98 (1H, s), 12.50 (1H, s).

### Example A9

### 15,21-Dimethyl-5-(morpholin-4-yl)-23-oxa-2,9,11,15,20,21,26-heptaazaheptacyclo[24.4.1.1\hat{ }{1,27}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione

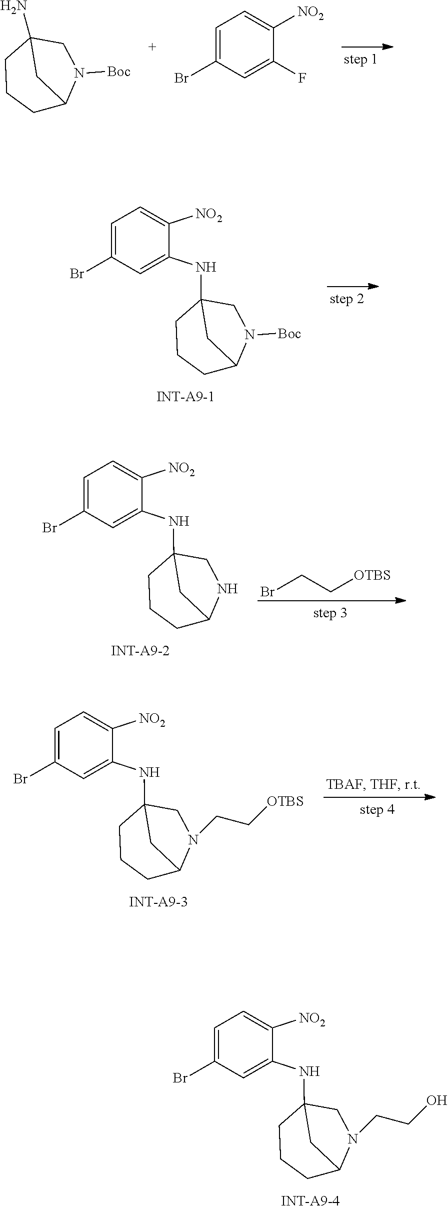

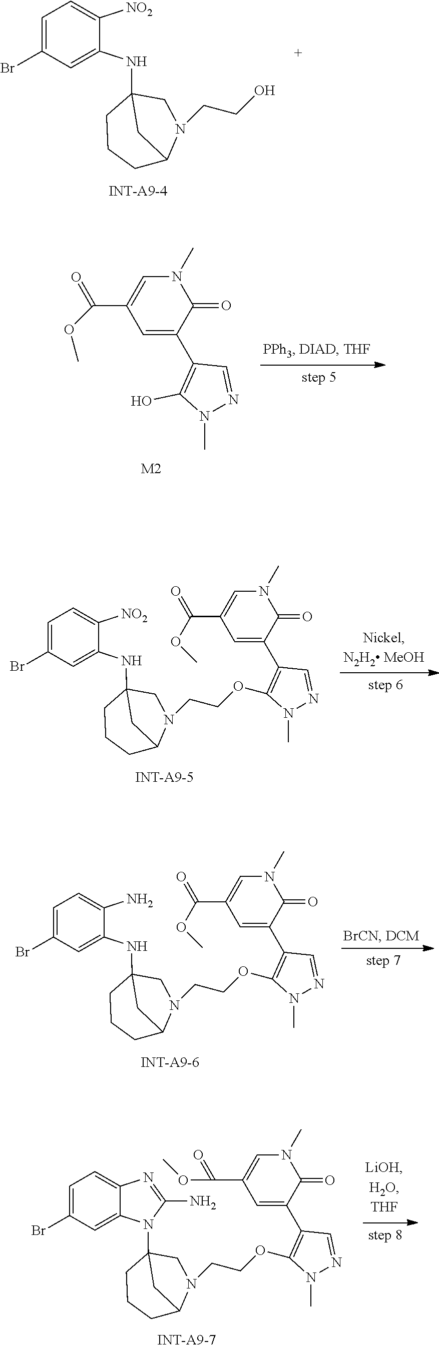 [SMILES: O=[N+]([O-])c1ccc(Br)cc1NC12CCCC(C1)N(CCO)C2 ; COC(=O)c1cc(-c2cnn(C)c2O)c(=O)n(C)c1 ; "M2" ; COC(=O)c1cc(-c2cnn(C)c2OCCN2CC3(Nc4cc(Br)ccc4[N+](=O)[O-])CCCC2C3)c(=O)n(C)c1 ; COC(=O)c1cc(-c2cnn(C)c2OCCN2CC3(Nc4cc(Br)ccc4N)CCCC2C3)c(=O)n(C)c1 ; COC(=O)c1cc(-c2cnn(C)c2OCCN2CC3(n4c(N)nc5ccc(Br)cc54)CCCC2C3)c(=O)n(C)c1]

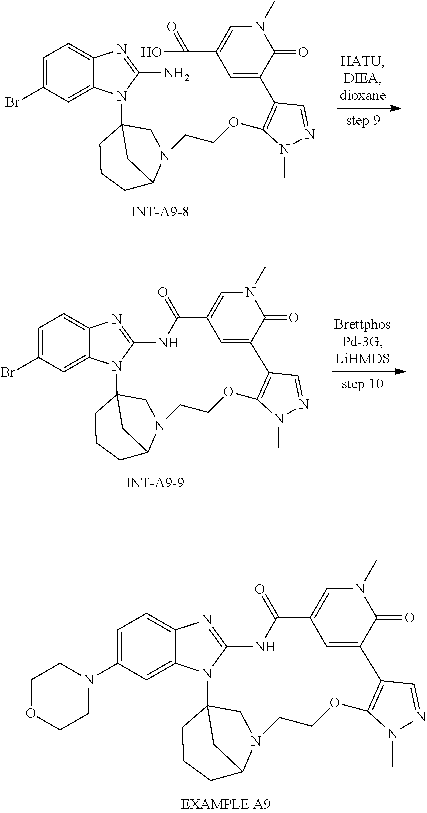 [SMILES: Cn1ncc(-c2cc(C(=O)O)cn(C)c2=O)c1OCCN1CC2(n3c(N)nc4ccc(Br)cc43)CCCC1C2 ; Cn1ncc2c1OCCN1CC3(CCCC1C3)n1c(nc3ccc(Br)cc31)NC(=O)c1cc-2c(=O)n(C)c1 ; Cn1ncc2c1OCCN1CC3(CCCC1C3)n1c(nc3ccc(N4CCOCC4)cc31)NC(=O)c1cc-2c(=O)n(C)c1]

Step 1. Preparation of tert-butyl 1-[(5-bromo-2-nitrophenyl)amino]-6-azabicyclo[3.2.1]octane-6-carboxylate (INT-A9-1). A mixture of tert-butyl 1-amino-6-azabicyclo[3.2.1]octane-6-carboxylate hydrochloride (1 g, 3.81 mmol, 1 equiv), 4-bromo-2-fluoro-1-nitrobenzene (2.51 g, 11.42 mmol, 3 equiv), and K₂CO₃ (2.10 g, 15.22 mmol, 4 equiv) in acetonitrile (20 mL) was stirred for 2 h at 60 °C. After being cooled to room temperature, the reaction was treated with water (200 mL) and the mixture was extracted with ethyl acetate (3×200 mL). The combined organic layers were washed with brine (3×100 mL), dried over anhydrous Na₂SO₄, and concentrated under reduced pressure. The residue was purified by silica gel chromatography eluted with petroleum ether/ethyl acetate (4/1) to give 1.25 g of tert-butyl 1-[(5-bromo-2-nitrophenyl)amino]-6-azabicyclo[3.2.1]octane-6-carboxylate (INT-A9-1) as a yellow solid (77%). LCMS: m/z (ESI), [M+H−56]⁺=371.85. ¹H NMR (DMSO-d₆, 400 MHz) δ 1.40-1.44 (10H, m), 1.48-1.75 (2H, m), 1.79-1.82 (1H, m), 1.98-2.00 (1H, m), 2.13-2.29 (3H, m), 3.23-3.33 (1H, m), 4.01-4.12 (2H, m), 6.92 (1H, d), 7.20 (1H, s), 8.03 (1H, d), 8.28 (1H, s).

Step 2. Preparation of N-(5-bromo-2-nitrophenyl)-6-azabicyclo[3.2.1]octan-1-amine (INT-A9-2). To a stirred mixture of tert-butyl 1-[(5-bromo-2-nitrophenyl)amino]-6-azabicyclo[3.2.1]octane-6-carboxylate (1.28 g, 3.00 mmol, 1 equiv) in dichloromethane (10 mL) was added TFA (10 mL) at room temperature. The resulting mixture was stirred for 2 h at room temperature and concentrated under reduced pressure. The residue was treated with saturated NaHCO₃ (aq.) solution (200 mL) and extracted with dichloromethane (3×200 mL). The combined organic layers were dried over anhydrous Na₂SO₄ and concentrated under reduced pressure to give 950 mg of N-(5-bromo-2-nitrophenyl)-6-azabicyclo[3.2.1]octan-1-amine (INT-A9-2) as a light yellow solid (97%). LCMS: m/z (ESI), [M+H]⁺=327.90. ¹H NMR (CD₃OD, 400 MHz) δ 1.51-1.64 (1H, m), 1.72-1.76 (2H, m), 1.80-1.95 (2H, m), 1.97 (1H, d), 2.21-2.33 (2H, m), 3.02-3.06 (1H, m), 3.54-3.65 (2H, m), 6.83-6.86 (1H, m), 7.14 (1H, d), 8.07 (1H, d).

Step 3. Preparation of N-(5-bromo-2-nitrophenyl)-6-{2-[(tert-butyldimethylsilyl)oxy]ethyl}-6-azabicyclo[3.2.1]octan-1-amine (INT-A9-3). A mixture of N-(5-bromo-2-nitrophenyl)-6-azabicyclo[3.2.1]octan-1-amine (580 mg, 1.78 mmol, 1 equiv), (2-bromoethoxy)(tert-butyl)dimethylsilane (850 mg, 3.56 mmol, 2 equiv), NaT (533 mg, 3.56 mmol, 2 equiv), and K₂CO₃ (491 mg, 3.56 mmol, 2 equiv) in N,N-dimethylformamide (10 mL) was stirred for 2 h at 60 °C under nitrogen atmosphere. After being cooled to room temperature, the reaction was quenched with water (100 mL) and the mixture was extracted with ethyl acetate (3×100 mL). The combined organic layers were washed with brine (3×100 mL), dried over anhydrous Na₂SO₄, and concentrated under reduced pressure. The residue was purified by reverse flash chromatography eluted with 20% to 100% acetonitrile in water to give 600 mg of N-(5-bromo-2-nitrophenyl)-6-{2-[(tert-butyldimethylsilyl)oxy]ethyl}-6-azabicyclo[3.2.1]octan-1-amine (INT-A9-3) as a light yellow solid (69%). LCMS: m/z (ESI), [M+H]⁺=484.51. ¹H NMR (CD₃OD, 400 MHz) δ 0.11 (6H, d), 0.93 (9H, s), 1.38-1.57 (2H, m), 1.62-1.78 (2H, m), 1.83-1.86 (2H, m), 2.00 (1H, d), 2.28 (1H, d), 2.33-2.42 (1H, m), 2.91 (2H, t), 3.36-3.42 (2H, m), 3.79-3.83 (2H, m), 6.82-6.86 (1H, m), 7.09 (1H, d), 8.07 (1H, d).

Step 4. Preparation of 2-{1-[(5-bromo-2-nitrophenyl)amino]-6-azabicyclo[3.2.1]octan-6-yl}ethanol (INT-A9-4). To a stirred mixture of N-(5-bromo-2-nitrophenyl)-6-{2-[(tert-butyldimethylsilyl)oxy]ethyl}-6-azabicyclo[3.2.1]octan-1-amine (598 mg, 1.23 mmol, 1 equiv) in tetrahydrofuran (9 mL) was added tetrabutylammonium fluoride (2 mL, 1 M in tetrahydrofuran) at room temperature. After being stirred at room temperature for 2 h, the reaction was quenched with water (100 mL) and the mixture was extracted with ethyl acetate (3×100 mL). The combined organic layers were washed with brine (5×100 mL), dried over anhydrous Na₂SO₄, and concentrated under reduced pressure to give 360 mg of 2-{1-[(5-bromo-2-nitrophenyl)amino]-6-azabicyclo[3.2.1]octan-6-yl}ethanol (INT-A9-4) as a yellow solid (78%). LCMS: m/z (ESI), [M+H]⁺=370.24.

Step 5. Preparation of methyl 5-[5-(2-{1-[(5-bromo-2-nitrophenyl)amino]-6-azabicyclo[3.2.1]octan-6-yl}ethoxy)-1-methylpyrazol-4-yl]-1-methyl-6-oxopyridine-3-carboxylate (INT-A9-5). To a stirred mixture of methyl 5-(5-hydroxy-1-methylpyrazol-4-yl)-1-methyl-6-oxopyridine-3-carboxylate (M2, 268 mg, 1.02 mmol, 1 equiv) and triphenylphosphine (714 mg, 2.72 mmol, 2.8 equiv) in tetrahydrofuran (10 mL) was added diisopropyl azodicarboxylate (491 mg, 2.43 mmol, 2.5 equiv) followed by the addition of 2-{1-[(5-bromo-2-nitrophenyl)amino]-6-azabicyclo[3.2.1]octan-6-yl}ethanol (360 mg, 0.97 mmol, 1 equiv) in tetrahydrofuran (3 mL) at 0 °C under nitrogen atmosphere. After being stirred at 0 °C for 30 mins, the reaction was quenched with water (100 mL) and the mixture was extracted with ethyl acetate (3×100 mL). The combined organic layers were washed with brine (3×100 mL), dried over anhydrous Na₂SO₄, and concentrated under reduced pressure. The residue was purified by silica gel chromatography eluted with petroleum ether/ethyl acetate (1/9) to give 470 mg of methyl 5-[5-(2-{1-[(5-bromo-2-nitrophenyl)amino]-6-azabicyclo[3.2.1]octan-6-yl}ethoxy)-1-methylpyrazol-4-yl]-1-methyl-6-oxopyridine-3-carboxylate (INT-A9-5) as a light yellow solid (78%). LCMS: m/z (ESI), [M+H]⁺=617.15. ¹H NMR (CD₃OD, 400 MHz) δ 1.39-1.51 (1H, m), 1.62-1.74 (2H, m), 1.85 (3H, q), 2.24-2.28 (2H, m), 3.12-3.16 (2H, m), 3.18-3.25 (3H, m), 3.64 (3H, s), 3.79 (3H, s), 3.83 (3H, s), 4.12-4.16 (2H, m), 6.82-6.86 (1H, m), 7.06 (1H, d), 7.96 (1H, s), 8.08 (1H, d), 8.25-8.33 (2H, m)

Step 6. Preparation of methyl 5-[5-(2-{1-[(2-amino-5-bromophenyl)amino]-6-azabicyclo[3.2.1]octan-6-yl}ethoxy)-1-methylpyrazol-4-yl]-1-methyl-6-oxopyridine-3-carboxylate (INT-A9-6). To a stirred mixture of methyl 5-[5-(2-{1-[(5-bromo-2-nitrophenyl)amino]-6-azabicyclo[3.2.1]octan-6-yl}ethoxy)-1-methylpyrazol-4-yl]-1-methyl-6-oxopyridine-3-carboxylate (460 mg, 0.75 mmol, 1 equiv) and Raney Ni (128 mg, 1.49 mmol, 2 equiv) in methanol (20 mL) was added and NH₂NH₂—H₂O (187 mg, 3.74 mmol, 5 equiv) at 0 °C. After being stirred for 2 h at room temperature, the reaction mixture was quenched by the addition of water (100 mL) and extracted with dichloromethane (3×100 mL). The combined organic layers were washed with brine (3×100 mL), dried over anhydrous Na₂SO₄, and concentrated under reduced pressure. The residue was purified by silica gel chromatography eluted with dichloromethane/methanol (20/1) to give 370 mg of methyl 5-[5-(2-{1-[(2-amino-5-bromophenyl)amino]-6-azabicyclo[3.2.1]octan-6-yl}ethoxy)-1-methylpyrazol-4-yl]-1-methyl-6-oxopyridine-3-carboxylate (INT-A9-6) as a reddish solid (84%). LCMS: m/z (EST), [M+H]⁺=586.95.

Step 7. Preparation of methyl 5-(5-{2-[1-(2-amino-6-bromo-1,3-benzodiazol-1-yl)-6-azabicyclo[3.2.1]octan-6-yl]ethoxy}-1-methylpyrazol-4-yl)-1-methyl-6-oxopyridine-3-carboxylate (INT-A9-7). A mixture of methyl 5-[5-(2-{1-[(2-amino-5-bromophenyl)amino]-6-azabicyclo[3.2.1]octan-6-yl}ethoxy)-1-methylpyrazol-4-yl]-1-methyl-6-oxopyridine-3-carboxylate (340 mg, 0.58 mmol, 1 equiv) and BrCN (92 mg, 0.87 mmol, 1.5 equiv) in EtOH (5 mL) was stirred for 4 h at room temperature. The reaction was quenched by sat. NaHCO₃ solution (100 mL) and extracted with dichloromethane (3×100 mL). The combined organic layers were washed with brine (3×100 mL), dried over anhydrous Na₂SO₄, and concentrated under reduced pressure. The residue was purified on silica gel column eluted with dichloromethane/methanol (20/1) to give 120 mg of methyl 5-(5-{2-[1-(2-amino-6-bromo-1,3-benzodiazol-1-yl)-6-azabicyclo[3.2.1]octan-6-yl]ethoxy}-1-methylpyrazol-4-yl)-1-methyl-6-oxopyridine-3-carboxylate (INT-A9-7) as a reddish solid (33%). LCMS: m/z (ESI), [M+H]⁺=612.15.

Step 8. Preparation of 5-(5-{2-[1-(2-amino-6-bromo-1,3-benzodiazol-1-yl)-6-azabicyclo[3.2.1]octan-6-yl]ethoxy}-1-methylpyrazol-4-yl)-1-methyl-6-oxopyridine-3-carboxylic acid (INT-A9-8). To a stirred solution of methyl 5-(5-{2-[1-(2-amino-6-bromo-1,3-benzodiazol-1-yl)-6-azabicyclo[3.2.1]octan-6-yl]ethoxy}-1-methylpyrazol-4-yl)-1-methyl-6-oxopyridine-3-carboxylate (120 mg, 0.20 mmol, 1 equiv) in THE (4 mL) was added LiOH (28 mg, 1.18 mmol, 6 equiv) in H₂O (1 mL) at room temperature. The resulting mixture was stirred for 2 h at room temperature and concentrated under reduced pressure. The residue was purified by reverse flash chromatography eluted with 20% to 100% acetonitrile in water to give 75 mg of 5-(5-{2-[1-(2-amino-6-bromo-1,3-benzodiazol-1-yl)-6-azabicyclo[3.2.1]octan-6-yl]ethoxy}-1-methylpyrazol-4-yl)-1-methyl-6-oxopyridine-3-carboxylic acid (INT-A9-8) as a white solid (63%). LCMS: m/z (ESI), [M+H]⁺=598.20.

Step 9. Preparation of 5-bromo-15,21-dimethyl-23-oxa-2,9,11,15,20,21,26-heptaazaheptacyclo[24.4.1.1\hat{ }{1,27}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (INT-A9-9). To a stirred solution of 5-(5-{2-[1-(2-amino-6-bromo-1,3-benzodiazol-1-yl)-6-azabicyclo[3.2.1]octan-6-yl]ethoxy}-1-methylpyrazol-4-yl)-1-methyl-6-oxopyridine-3-carboxylic acid (70 mg, 0.12 mmol, 1 equiv) in dioxane (2 mL) were added N,N-diisopropylethylamine (45 mg, 0.35 mmol, 3 equiv) and N,N,N,N-Tetramethyl-O-(7-azabenzotriazol-1-yl)uronium hexafluorophospate (66 mg, 0.17 mmol, 1.5 equiv) at room temperature. After being stirred for 2 h at room temperature, the reaction was quenched with water (80 mL) and the mixture was extracted with dichloromethane (2×80 mL). The combined organic layers were washed with brine (3×80 mL), dried over anhydrous Na₂SO₄, and concentrated under reduced pressure. The residue was purified by prep-TLC eluted with dichloromethane/methanol (20/1) to give 65 mg of 5-bromo-15,21-dimethyl-23-oxa-2,9,11,15,20,21,26-heptaazaheptacyclo[24.4.1.1\hat{ }{1,27}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (INT-A9-9) as a brown solid (95%). LCMS: m/z (ESI), [M+H]⁺=580.00.

Step 10. Preparation of 15,21-dimethyl-5-(morpholin-4-yl)-23-oxa-2,9,11,15,20,21,26-heptaazaheptacyclo[24.4.1.1\hat{ }{1,27}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (EXAMPLE A9). To a stirred mixture of 5-bromo-15,21-dimethyl-23-oxa-2,9,11,15,20,21,26-heptaazaheptacyclo[24.4.1.1\hat{ }{1,27}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (50 mg, 0.09 mmol, 1 equiv), morpholine (30 mg, 0.34 mmol, 4 equiv) and BrettPhos Pd G₃ (23 mg, 0.03 mmol, 0.3 equiv) in dioxane (3 mL) was added LiHMDS (0.34 mL, 0.34 mmol, 4 equiv) dropwise at room temperature under nitrogen atmosphere. The reaction mixture was stirred at 60 °C for 30 mins. After being cooled to room temperature, the reaction was quenched with water (50 mL) and the mixture was extracted with ethyl acetate (3×50 mL). The combined organic layers were washed with brine (3×50 mL), dried over anhydrous Na₂SO₄, and concentrated under reduced pressure. The residue was purified by prep-HPLC with XBridge Shield RP18 OBD column using water containing 10 mmol/L NH₄HCO₃ and 0.10% NH₃—H₂O and acetonitrile as mobile phase. Fractions containing the desired compound were evaporated to dryness to give 13 mg of 15,21-dimethyl-5-(morpholin-4-yl)-23-oxa-2,9,11,15,20,21,26-heptaazaheptacyclo[24.4.1.1\hat{ }{1,27}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (EXAMPLE A9) as a white solid (25%). LCMS: m/z (EST), [M+H]⁺=585.35. ¹H NMR (CD₃OD, 400 MHz) δ 1.63-1.69 (1H, m), 1.85-1.92 (2H, m), 2.02-2.08 (1H, m), 2.15 (1H, d), 2.26 (1H, q), 2.60-2.65 (1H, m), 2.82 (1H, d), 3.15-3.23 (4H, m), 3.39 (1H, m), 3.47-3.58 (2H, m), 3.68 (3H, s), 3.85 (3H, s), 3.90 (4H, t), 4.41-4.49 (3H, m), 4.61-4.68 (1H, m), 7.00 (1H, m), 7.25 (1H, d), 7.38 (1H, d), 8.20 (1H, s), 8.49 (1H, s), 9.05 (1H, d).

### Example A10

### 1,15,21-Trimethyl-5-(morpholin-4-yl)-23-oxa-2,9,11,15,20,21,26-heptaazahexacyclo[24.3.1.1\hat{ }{3,17}.0\hat{ }{2,10}.0\hat{ }{3,8}.0\hat{ }{18,22}]hentriaconta-3,5,7,9,13,17(31),18(22),19-octaene-12,16-dione

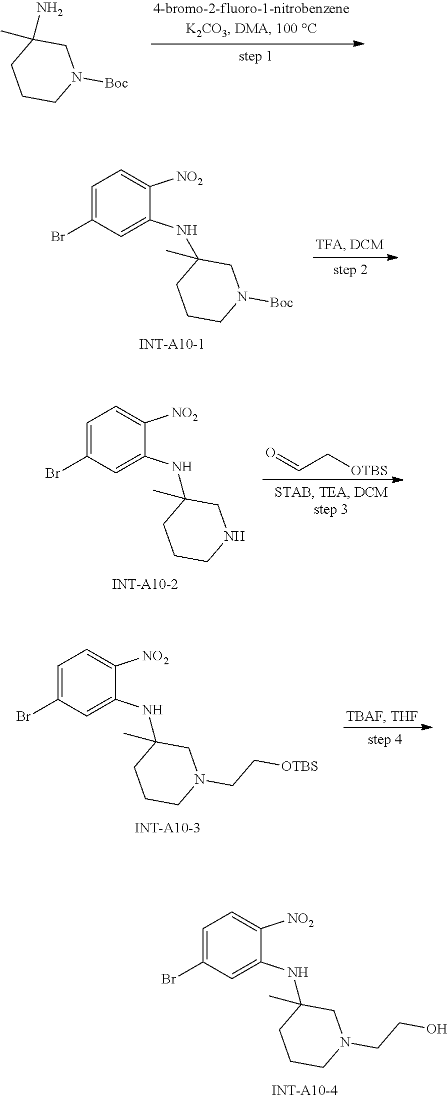

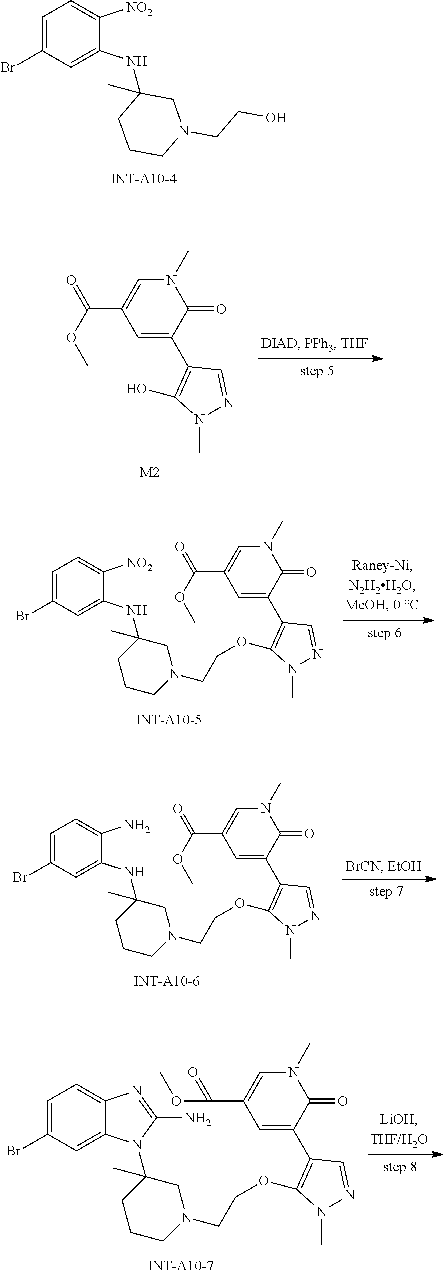 [SMILES: CC1(Nc2cc(Br)ccc2[N+](=O)[O-])CCCN(CCO)C1 ; COC(=O)c1cc(-c2cnn(C)c2O)c(=O)n(C)c1 ; "M2" ; COC(=O)c1cc(-c2cnn(C)c2OCCN2CCCC(C)(Nc3cc(Br)ccc3[N+](=O)[O-])C2)c(=O)n(C)c1 ; COC(=O)c1cc(-c2cnn(C)c2OCCN2CCCC(C)(Nc3cc(Br)ccc3N)C2)c(=O)n(C)c1 ; COC(=O)c1cc(-c2cnn(C)c2OCCN2CCCC(C)(n3c(N)nc4ccc(Br)cc43)C2)c(=O)n(C)c1]

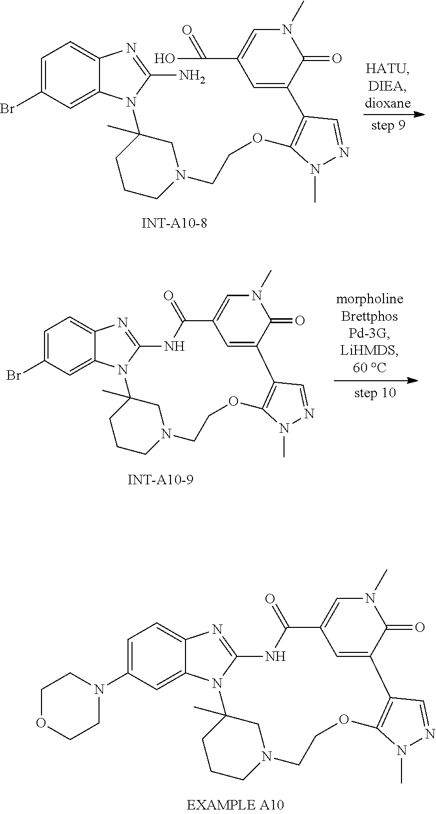 [SMILES: Cn1ncc(-c2cc(C(=O)O)cn(C)c2=O)c1OCCN1CCCC(C)(n2c(N)nc3ccc(Br)cc32)C1 ; Cn1ncc2c1OCCN1CCCC(C)(C1)n1c(nc3ccc(Br)cc31)NC(=O)c1cc-2c(=O)n(C)c1 ; Cn1ncc2c1OCCN1CCCC(C)(C1)n1c(nc3ccc(N4CCOCC4)cc31)NC(=O)c1cc-2c(=O)n(C)c1]

Step 1. Preparation of the tert-butyl 3-((5-bromo-2-nitrophenyl)amino-3-methylpiperidine-1-carboxylate (INT-A10-1). A mixture of 4-bromo-2-fluoro-1-nitrobenzene (6.16 g, 27.99 mmol, 3 equiv), K₂CO₃ (3.8 g, 27.99 mmol, 3 equiv), and tert-butyl 3-amino-3-methylpiperidine-1-carboxylate (2 g, 9.33 mmol, 1.00 equiv) in NN-dimethylacetamide (40 mL) was stirred for 12 h at 100 °C under nitrogen atmosphere and then cooled to 25 °C. The reaction was quenched with water (300 mL) and the mixture was extracted with ethyl acetate (3×200 mL). The combined organic layers were washed with brine (3×300 mL), dried over anhydrous Na₂SO₄, and concentrated under reduced pressure. The residue was purified by prep-TLC eluted with petroleum ether/ethyl acetate (3/1) to give 3.5 g of tert-butyl 3-[(5-bromo-2-nitrophenyl)amino]-3-methylpiperidine-1-carboxylate (INT-A10-1) as a yellow oil (91%). ¹H NMR (DMSO-d, 400 MHz) δ 1.23 (9H, s), 1.44 (3H, s), 1.54 (2H, s), 1.64 (1H, s), 2.15 (1H, s), 2.78 (1H, s), 2.98 (1H, s), 3.88 (1H, s), 4.16-4.14 (1H, d), 6.86-6.93 (1H, m), 7.30 (1H, d), 8.04-7.98 (1H, d), 8.24 (1H, s).

Step 2. Preparation of N-(5-bromo-2-nitrophenyl)-3-methylpiperidin-3-amine (INT-A 10-2). A mixture of trifluoroacetic acid (5 mL, 67.31 mmol, 7 equiv) and tert-butyl 3-[(5-bromo-2-nitrophenyl)amino]-3-methylpiperidine-1-carboxylate (3.5 g, 8.44 mmol, 1 equiv) in CH₂Cl₂ (15 mL) was stirred for 2 h at room temperature under nitrogen atmosphere and concentrated under reduced pressure. The residue was treated with saturated NaHCO₃ (aq.) (100 mL) and extracted with ethyl acetate (3×100 mL). The combined organic layers were washed with brine (3×100 mL), dried over anhydrous Na₂SO₄, and concentrated under reduced pressure to give 2.4 g of N-(5-bromo-2-nitrophenyl)-3-methylpiperidin-3-amine (INT-A10-2) as a yellow solid (89%). ¹H NMR (DMSO-d₆, 400 MHz) δ 1.51 (3H, s), 1.85-1.83 (3H, m), 2.03-2.0 (1H, t), 2.92-2.89 (1H, m), 3.09-3.05 (1H, d), 3.17-3.14 (1H, m), 3.85-3.82 (1H, d), 6.96-6.95 (1H, m), 7.34-7.31 (1H, d), 8.07-8.05 (1H, d), 8.18 (1H, s)

Step 3. Preparation of N-(5-bromo-2-nitrophenyl)-1-(2-((tert-butyldimethylsilyl)oxy)ethyl)-3-methylpiperidin-3-amine (INT-A10-3). To a stirred mixture of N-(5-bromo-2-nitrophenyl)-3-methylpiperidin-3-amine (1 g, 3.81 mmol, 1 equiv), 2-[(tert-butyldimethylsilyl)oxy]acetaldehyde (2 g, 15.27 mmol, 4 equiv) and trimethylamine (1.5 g, 15.27 mmol, 4 equiv) in CH₂Cl₂ (50 mL) was added NaBH(OAc)₃ (1.6 g, 7.63 mmol, 2 equiv) at 0 °C under nitrogen atmosphere. The mixture was stirred for 2 h at room temperature. The reaction was quenched with water (100 mL) and the mixture was extracted with dichloromethane (3×100 mL). The combined organic layers were washed with brine (3×100 mL), dried over anhydrous Na₂SO₄, and concentrated under reduced pressure. The residue was purified by silica gel chromatography eluted with petroleum ether/ethyl acetate (5/2) to give 840 mg of N-(5-bromo-2-nitrophenyl)-1-{2-[(tert-butyldimethylsilyl)oxy]ethyl}-3-methylpiperidin-3-amine (INT-A10-3) as a yellow oil (46%). ¹H NMR (DMSO-d₆, 400 MHz) δ 0.06 (6H, s), 0.86 (9H, s), 1.28-1.25 (1H, td), 1.39 (5H, s), 2.00-2.17 (2H, m), 2.23-2.20 (1H, d), 2.83-2.81 (1H, d), 2.93-2.9 (1H, d), 3.77-3.74 (2H, td), 6.82-6.80 (1H, m), 7.22-7.19 (1H, d), 8.01-7.98 (1H, d), 8.78 (1H, s).

Step 4. Preparation of 2-(3-((5-bromo-2-nitrophenyl)amino)-3-methylpiperidin-1-yl)ethan-1-ol (INT-A10-4). A mixture of N-(5-bromo-2-nitrophenyl)-1-{2-[(tert-butyldimethylsilyl)oxy]ethyl}-3-methylpiperidin-3-amine (700 mg, 1.48 mmol, 1 equiv) and tetrabutylammonium fluoride (774.7 mg, 2.96 mmol, 2.0 equiv) in tetrahydrofuran (7 mL) was stirred for 4 h at room temperature under nitrogen atmosphere. The reaction mixture was treated with water (30 mL) and extracted with dichloromethane (3×30 mL). The combined organic layers were washed with brine (3×30 mL), dried over anhydrous Na₂SO₄, and concentrated under reduced pressure. The residue was purified by prep-TLC eluted with CH₂Cl₂/methanol (12/1) to give 500 mg of 2-{3-[(5-bromo-2-nitrophenyl)amino]-3-methylpiperidin-1-yl}ethanol (INT-A10-4) as a brown solid (94%). ¹H NMR (DMSO-d₆, 400 MHz) δ 1.19-1.16 (1H, t), 1.29-1.26 (1H, t), 1.41 (4H, s), 1.98-2.13 (3H, m), 2.25-2.23 (1H, d), 2.85-2.83 (2H, d), 3.59-3.57 (2H, t), 4.40-4.36 (1H, t), 6.83-6.80 (1H, d), 7.23-7.21 (1H, d), 8.03-8.01 (1H, d), 8.80 (1H, s).

Step 5. Preparation of methyl 5-[5-(2-{3-[(5-bromo-2-nitrophenyl)amino]-3-methylpiperidin-1-yl}ethoxy)-1-methylpyrazol-4-yl]-1-methyl-6-oxopyridine-3-carboxylate (INT-A10-5). To a stirred mixture of methyl 5-(5-hydroxy-1-methylpyrazol-4-yl)-1-methyl-6-oxopyridine-3-carboxylate (M2, 350 mg, 1.33 mmol, 1 equiv) and triphenylphosphine (1046 mg, 3.99 mmol, 3 equiv) in tetrahydrofuran (10 mL) were added diisopropyl azodicarboxylate (806 mg, 3.99 mmol, 3 equiv) and 2-{3-[(5-bromo-2-nitrophenyl)amino]-3-methylpiperidin-1-yl}ethanol (523 mg, 1.463 mmol, 1.1 equiv) at 0 °C under nitrogen atmosphere. The mixture was stirred for 2 h. The reaction was quenched with water (100 mL) and the mixture was extracted with dichloromethane (3×100 mL). The combined organic layers were washed with brine (3×100 mL), dried over anhydrous Na₂SO₄, and concentrated under reduced pressure. The residue was purified by C18 FLASH using water and acetonitrile as mobile phase. Fractions containing the desired compound were evaporated to dryness to give 450 mg of methyl 5-[5-(2-{3-[(5-bromo-2-nitrophenyl)amino]-3-methylpiperidin-1-yl}ethoxy)-1-methylpyrazol-4-yl]-1-methyl-6-oxopyridine-3-carboxylate (INT-A10-5) as a yellow solid (56%). ¹H NMR (DMSO-d₆, 400 MHz) δ 1.41 (6H, s), 2.06-2.29 (3H, m), 2.74-2.94 (3H, m), 3.58 (3H, s), 3.69 (3H, s), 3.76 (3H, s), 4.10-4.06 (2H, m), 6.83-6.78 (1H, d), 7.23-7.20 (1H, d), 7.95 (1H, s), 8.01-7.98 (1H, d), 8.11-8.02 (1H, d), 8.44 (1H, d), 8.74 (1H, s).

Step 6. Preparation of methyl 5-(5-(2-(3-((2-amino-5-bromophenyl)amino)-3-methylpiperidin-1-yl)ethoxy)-1-methyl-1H-pyrazol-4-yl)-1-methyl-6-oxo-1,6-dihydropyridine-3-carboxylate (INT-A10-6). A mixture of methyl 5-[5-(2-{3-[(5-bromo-2-nitrophenyl)amino]-3-methylpiperidin-1-yl}ethoxy)-1-methylpyrazol-4-yl]-1-methyl-6-oxopyridine-3-carboxylate (INT-A10-5, 430 mg, 0.71 mmol, 1 equiv), Raney-Ni (61 mg, 0.71 mmol, 1 equiv) and NH₂NH₂—H₂O (71 mg, 1.42 mmol, 2 equiv) in methanol (6 mL) was stirred for 0.5 h at 0 °C under nitrogen atmosphere. The resulting mixture was filtered. The filter cake was washed with methanol (50 mL). The solution was concentrated under reduced pressure to give 340 mg of methyl 5-[5-(2-{3-[(2-amino-5-bromophenyl)amino]-3-methylpiperidin-1-yl}ethoxy)-1-methylpyrazol-4-yl]-1-methyl-6-oxopyridine-3-carboxylate (INT-A10-6) as a yellow solid (83%). LCMS: m/z (ESI), [M+H]⁺=573.20.

Step 7. Preparation of methyl 5-(5-(2-(3-(2-amino-6-bromo-1H-benzo[d]imidazol-1-yl)-3-methylpiperidin-1-yl)ethoxy)-1-methyl-1H-pyrazol-4-yl)-1-methyl-6-oxo-1,6-dihydropyridine-3-carboxylate (INT-A10-7). A mixture of methyl 5-[5-(2-{3-[(2-amino-5-bromophenyl)amino]-3-methylpiperidin-1-yl}ethoxy)-1-methylpyrazol-4-yl]-1-methyl-6-oxopyridine-3-carboxylate (340 mg, 0.59 mmol, 1 equiv) and BrCN (69 mg, 0.65 mmol, 1.1 equiv) in EtOH (5 mL) was stirred overnight at room temperature under nitrogen atmosphere. The resulting mixture was concentrated under reduced pressure. The residue was purified by prep-TLC eluted with CH₂Cl₂/methanol (12/1) to give 300 mg of methyl 5-(5-{2-[3-(2-amino-6-bromo-1,3-benzodiazol-1-yl)-3-methylpiperidin-1-yl]ethoxy}-1-methylpyrazol-4-yl)-1-methyl-6-oxopyridine-3-carboxylate (INT-A10-7) as a brown solid (84%). ¹H NMR (DMSO-d₆, 400 MHz) δ 0.85-0.84 (1H, m), 0.98-1.09 (1H, m), 1.25-1.23 (4H, m), 1.49-1.47 (1H, m), 1.59 (4H, s), 2.20-2.07 (1H, m), 2.44-2.40 (1H, d), 2.79-2.67 (2H, m), 2.94-2.90 (1H, m), 3.15-3.12 (2H, m), 3.59 (3H, s), 3.71 (3H, s), 3.76 (3H, s), 4.21-4.06 (2H, m), 7.03-7.12 (2H, m), 7.35 (2H, s), 7.50-7.45 (1H, d), 7.95 (1H, s), 8.11-8.05 (1H, d), 8.47-8.45 (1H, d).

Step 8. Preparation of 5-(5-(2-(3-(2-amino-6-bromo-1H-benzo[d]imidazol-1-yl)-3-methylpiperidin-1-yl)ethoxy)-1-methyl-1H-pyrazol-4-yl)-1-methyl-6-oxo-1,6-dihydropyridine-3-carboxylic acid (INT-A10-8). A mixture of methyl 5-(5-{2-[3-(2-amino-6-bromo-1,3-benzodiazol-1-yl)-3-methylpiperidin-1-yl]ethoxy}-1-methylpyrazol-4-yl)-1-methyl-6-oxopyridine-3-carboxylate (290 mg, 0.48 mmol, 1 equiv) and LiOH·H₂O (40 mg, 0.970 mmol, 2 equiv) in THF/H₂O (2 mL/0.5 mL) was stirred for 2 h at room temperature under nitrogen atmosphere. The resulting mixture was concentrated under reduced pressure. The residue was purified by C18 FLASH using water and acetonitrile as mobile phase. Fractions containing the desired compound were evaporated to dryness to give 230 mg of 5-(5-{2-[3-(2-amino-6-bromo-1,3-benzodiazol-1-yl)-3-methylpiperidin-1-yl]ethoxy}-1-methylpyrazol-4-yl)-1-methyl-6-oxopyridine-3-carboxylic acid (INT-A10-8) as a brown solid (81%). ¹H NMR (400 MHz, DMSO-d₆) δ 1.21-1.31 (1H, m), 1.49 (1H, t), 1.58 (4H, s), 2.14 (1H, t), 2.42 (1H, d), 2.70-2.84 (2H, m), 2.87-2.98 (1H, m), 3.13 (1H, d), 3.50 (3H, s), 3.69 (3H, s), 4.04-4.17 (2H, m), 7.08 (2H, d), 7.48 (1H, s), 7.54 (2H, s), 7.88 (1H, s), 7.99 (1H, t), 8.18 (1H, d).

Step 9. Preparation of 5-bromo-1,15,21-trimethyl-23-oxa-2,9,11,15,20,21,26-heptaazahexacyclo[24.3.1.1\hat{ }{13,17}.0\hat{ }{2,10}.0\hat{ }{3,8}.0\hat{ }{18,22}]hentriaconta-3,5,7,9,13,17(31),18(22),19-octaene-12,16-dione (INT-A10-9). A mixture of 5-(5-{2-[3-(2-amino-6-bromo-1,3-benzodiazol-1-yl)-3-methylpiperidin-1-yl]ethoxy}-1-methylpyrazol-4-yl)-1-methyl-6-oxopyridine-3-carboxylic acid (230 mg, 0.39 mmol, 1 equiv), N,N-diisopropylethylamine (152.58 mg, 1.182 mmol, 3 equiv) and N,N,N,N-tetramethyl-O-(7-azabenzotriazol-1-yl)uronium hexafluorophospate (224.44 mg, 0.59 mmol, 1.5 equiv) in dioxane (5 mL) was stirred for 2 h at 60 °C under nitrogen atmosphere. The mixture was cooled to room temperature and concentrated under reduced pressure. The residue was purified by Pprep-TLC eluted with dichloromethane/methanol (10/1) to give 180 mg of 5-bromo-1,15,21-trimethyl-23-oxa-2,9,11,15,20,21,26-heptaazahexacyclo[24.3.1.1\hat{ }{13,17}.0\hat{ }{2,10}.0\hat{ }{3,8}.0\hat{ }{18,22}]hentriaconta-3,5,7,9,13,17(31),18(22),19-octaene-12,16-dione (INT-A10-9) as a brown solid (80%). LCMS: m/z (ESI), [M+H]⁺=566.10.

Step 10. Preparation of 1,15,21-trimethyl-5-(morpholin-4-yl)-23-oxa-2,9,11,15,20,21,26-heptaazahexacyclo[24.3.1.1\hat{ }{13,17}.0\hat{ }{2,10}.0\hat{ }{3,8}.0\hat{ }{18,22}]hentriaconta-3,5,7,9,13,17(31),18(22),19-octaene-12,16-dione (EXAMPLE A10). To a stirred mixture of 5-bromo-1,15,21-trimethyl-23-oxa-2,9,11,15,20,21,26-heptaazahexacyclo[24.3.1.1\hat{ }{13,17}.0\hat{ }{2,10}.0\hat{ }{3,8}.0\hat{ }{18,22}]hentriaconta-3,5,7,9,13,17(31),18(22),19-octaene-12,16-dione (90 mg, 0.16 mmol, 1 equiv), BrettPhos Pd G₃ (43 mg, 0.05 mmol, 0.3 equiv) and morpholine (55 mg, 0.63 mmol, 4 equiv) in dioxane (4 mL) was added LiHMDS (0.95 mL, 0.95 mmol, 6.0 equiv) at room temperature under nitrogen atmosphere. The mixture was stirred for 1 h at 60 °C, cooled to room temperature, treated with water (30 mL), and extracted with ethyl acetate (3×30 mL). The combined organic layers were washed with brine (3×30 mL), dried over anhydrous Na₂SO₄, and concentrated under reduced pressure. The residue was purified by prep-HPLC with XBridge Shield RP18 OBD column using water containing 10 mmol/L NH₄HCO₃+0.10% NH₃·H₂O and acetonitrile as mobile phase. Fractions containing the desired compound were evaporated to dryness to give 12 mg of 1,15,21-trimethyl-5-(morpholin-4-yl)-23-oxa-2,9,11,15,20,21,26-heptaazahexacyclo[24.3.1.1\hat{ }{13,17}.0\hat{ }{2,10}.0\hat{ }{3,8}.0\hat{ }{18,22}]hentriaconta-3,5,7,9,13,17(31),18(22),19-octaene-12,16-dione (EXAMPLE A10) as a white solid (13%). LCMS: m/z (ESI), [M+H]⁺=573.50. ¹H NMR (DMSO-d₆, 400 MHz) δ 1.89-1.85 (5H, m), 2.12-2.08 (1H, m), 2.30 (1H, s), 2.72-2.69 (1H, m), 2.92 (1H, d), 3.08-3.06 (3H, m), 3.29 (2H, s), 3.62 (3H, s), 3.72 (2H, s), 3.76-3.74 (3H, m), 4.62 (1H, t), 5.42-5.40 (1H, d), 6.93-6.90 (1H, m), 7.32 (1H, d), 7.48 (1H, d), 8.21-8.37 (1H, m), 8.74 (1H, d), 12.80 (1H, s).

### Example A11

### 5,13,27-Trimethyl-17-(morpholin-4-yl)-7-oxa-4,5,10,14,21,23,27-heptaazahexacyclo[23.3.1.1\hat{ }{10,13}.0\hat{ }{2,6}.0\hat{ }{14,22}.0\hat{ }{15,20}]triaconta-1(29),2(6),3,15,17,19,21,25-octaene-24,28-dione

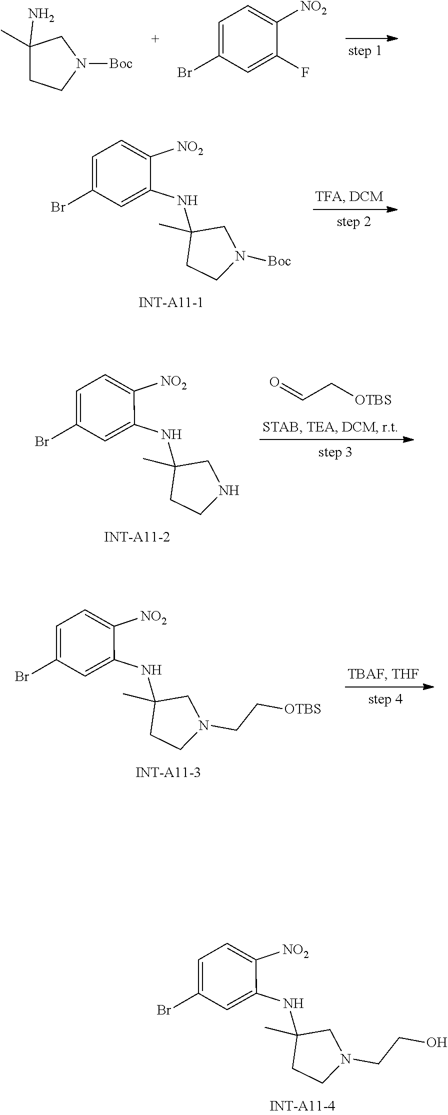

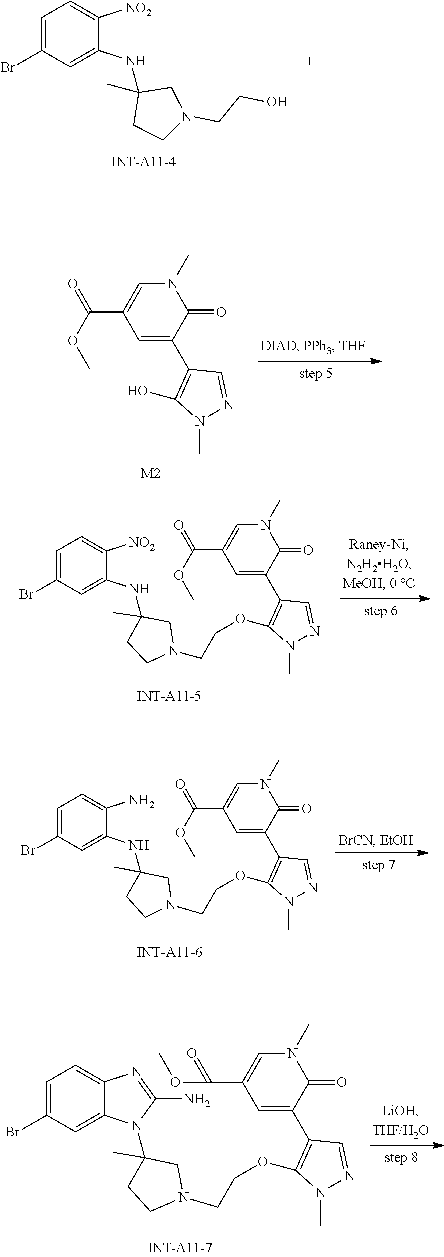 [SMILES: CC1(Nc2cc(Br)ccc2[N+](=O)[O-])CCN(CCO)C1 ; COC(=O)c1cc(-c2cnn(C)c2O)c(=O)n(C)c1 ; "M2" ; COC(=O)c1cc(-c2cnn(C)c2OCCN2CCC(C)(Nc3cc(Br)ccc3[N+](=O)[O-])C2)c(=O)n(C)c1 ; COC(=O)c1cc(-c2cnn(C)c2OCCN2CCC(C)(Nc3cc(Br)ccc3N)C2)c(=O)n(C)c1 ; COC(=O)c1cc(-c2cnn(C)c2OCCN2CCC(C)(n3c(N)nc4ccc(Br)cc43)C2)c(=O)n(C)c1]

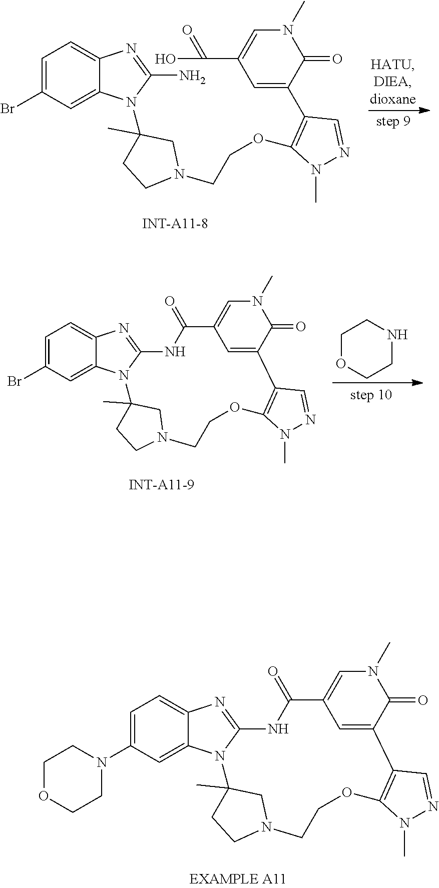

Step 1. Preparation of tert-butyl 3-[(5-bromo-2-nitrophenyl)amino]-3-methylpyrrolidine-1-carboxylate (INT-A11-1). A mixture of 4-bromo-2-fluoro-1-nitrobenzene (2 g, 9.1 mmol, 3 equiv), K₂CO₃ (1.26 g, 9.09 mmol, 3 equiv), and tert-butyl 3-amino-3-methylpyrrolidine-1-carboxylate (0.61 g, 3.03 mmol, 1 equiv) in acetonitrile (20 mL) was stirred for 2 h at 100 °C under nitrogen atmosphere and cooled to room temperature. The reaction was quenched with water (200 mL) and the mixture was extracted with ethyl acetate (3×100 mL). The combined organic layers were washed with brine (3×100 mL), dried over anhydrous Na₂SO₄, and concentrated under reduced pressure. The residue was purified by Prep-TLC eluted with petroleum ether/ethyl acetate (4/1) to give 1.2 g of tert-butyl 3-[(5-bromo-2-nitrophenyl)amino]-3-methylpyrrolidine-1-carboxylate (INT-A11-1) as a yellow solid (80%). LCMS: m/z (ES+), [M+H−56]⁺=344.00.

Step 2. Preparation of N-(5-bromo-2-nitrophenyl)-3-methylpyrrolidin-3-amine (INT-A11-2). A mixture of tert-butyl 3-[(5-bromo-2-nitrophenyl)amino]-3-methylpyrrolidine-1-carboxylate (2.1 g, 5.25 mmol, 1 equiv) in trifluoroacetic acid (4 mL) and dichloromethane (16 mL) was stirred for 2 h at room temperature under nitrogen atmosphere. The mixture was concentrated under reduced pressure and treated with saturated NaHCO₃ (aq.) solution (100 mL). The mixture was extracted with ethyl acetate (3×100 mL). The combined organic layers were washed with brine (3×100 mL), dried over anhydrous Na₂SO₄, and concentrated under reduced pressure. The residue was purified by Prep-TLC eluted with dichloromethane/methanol (10/1) to give 1.3 g of N-(5-bromo-2-nitrophenyl)-3-methylpyrrolidin-3-amine (INT-A11-2) as a yellow solid (74%). LCMS: m/z (ES+), [M+H]⁺=300.20.

Step 3. Preparation of N-(5-bromo-2-nitrophenyl)-1-{2-[(tert-butyldimethylsilyl)oxy]ethyl}-3-methylpyrrolidin-3-amine (INT-A11-3). To a stirred solution of N-(5-bromo-2-nitrophenyl)-3-methylpyrrolidin-3-amine (1.2 g, 4 mmol, 1 equiv) and 2-[(tert-butyldimethylsilyl)oxy]acetaldehyde (2.8 g, 16 mmol, 4 equiv) in dichloromethane (10 mL) were added trimethylamine (1.62 g, 16 mmol, 4 equiv) and NaBH(OAc)₃ (1.69 g, 8 mmol, 2 equiv) at room temperature under nitrogen atmosphere. The resulting mixture was stirred overnight at 60 °C and cooled to room temperature. The reaction was quenched with water (10 mL) and the mixture was extracted with ethyl acetate (3×10 mL). The combined organic layers were washed with brine (3×10 mL), dried over anhydrous Na₂SO₄, and concentrated under reduced pressure. The residue was purified by prep-HPLC with XBridge Shield RP18 OBD column using water containing 10 mmol/L NH₄HCO₃ and 0.1% NH₃·H₂O and acetonitrile as mobile phase. Fractions containing the desired compound were evaporated to dryness to give 1.24 g of N-(5-bromo-2-nitrophenyl)-1-{2-[(tert-butyldimethylsilyl)oxy]ethyl}-3-methylpyrrolidin-3-amine (INT-A11-3) as a yellow oil (61%). LCMS: m/z (ES+), [M+H]⁺=458.50

Step 4. Preparation of 2-{3-[(5-bromo-2-nitrophenyl)amino]-3-methylpyrrolidin-1-yl}ethanol (INT-A11-4). A mixture of N-(5-bromo-2-nitrophenyl)-1-{2-[(tert-butyldimethylsilyl)oxy]ethyl}-3-methylpyrrolidin-3-amine (1.24 g, 2.71 mmol, 1 equiv) and tetrabutylammonium fluoride (1.41 g, 5.41 mmol, 2 equiv) in tetrahydrofuran (12 mL) was stirred for 2 h at room temperature under nitrogen atmosphere. The reaction was quenched with sat. NaHCO₃ (aq.) solution (50 mL) at room temperature and the mixture was extracted with dichloromethane (3×100 mL). The combined organic layers were washed with brine (3×100 mL), dried over anhydrous Na₂SO₄, and concentrated under reduced pressure. The residue was purified by Prep-TLC eluted with dichloromethane/methanol (10/1) to give 800 mg of 2-{3-[(5-bromo-2-nitrophenyl)amino]-3-methylpyrrolidin-1-yl}ethanol (INT-A11-4) as a yellow oil (69%). LCMS: m/z (ES+), [M+H]⁺=344.00.

Step 5. Preparation of methyl 5-[5-(2-{3-[(5-bromo-2-nitrophenyl)amino]-3-methylpyrrolidin-1-yl}ethoxy)-1-methylpyrazol-4-yl]-1-methyl-6-oxopyridine-3-carboxylate (INT-A11-5). To a stirred mixture of 2-{3-[(5-bromo-2-nitrophenyl)amino]-3-methylpyrrolidin-1-yl}ethanol (800 mg, 2.32 mmol, 1 equiv) and triphenylphosphine (1829 mg, 7 mmol, 3 equiv) in tetrahydrofuran (15 mL) were added methyl 5-(5-hydroxy-1-methylpyrazol-4-yl)-1-methyl-6-oxopyridine-3-carboxylate (M2, 611 mg, 2.32 mmol, 1.0 equiv) and diisopropyl azodicarboxylate (1410 mg, 7 mmol, 3 equiv) at room temperature under nitrogen atmosphere. The resulting mixture was stirred for 2 h at room temperature. The reaction was quenched with water (100 mL) and the mixture was extracted with dichloromethane (3×100 mL). The combined organic layers were washed with brine (3×100 mL), dried over anhydrous Na₂SO₄, and concentrated under reduced pressure. The residue was purified by Prep-TLC eluted with dichloromethane/methanol (10/1) to give 380 mg of methyl 5-[5-(2-{3-[(5-bromo-2-nitrophenyl)amino]-3-methylpyrrolidin-1-yl}ethoxy)-1-methylpyrazol-4-yl]-1-methyl-6-oxopyridine-3-carboxylate (INT-A11-5) as a yellow oil (25%). LCMS: m/z (ES+), [M+H]⁺=589.00.

Step 6. Preparation of methyl 5-[5-(2-{3-[(2-amino-5-bromophenyl)amino]-3-methylpyrrolidin-1-yl}ethoxy)-1-methylpyrazol-4-yl]-1-methyl-6-oxopyridine-3-carboxylate (INT-A11-6). To a stirred mixture of methyl 5-[5-(2-{3-[(5-bromo-2-nitrophenyl)amino]-3-methylpyrrolidin-1-yl}ethoxy)-1-methylpyrazol-4-yl]-1-methyl-6-oxopyridine-3-carboxylate (380 mg, 0.65 mmol, 1 equiv) and Raney Ni (55.23 mg, 0.65 mmol, 1 equiv) in methanol (6 mL) was added NH₂NH₂H₂O (129.09 mg, 2.58 mmol, 4 equiv) dropwise at 0 °C. The resulting mixture was stirred for 1.5 h at room temperature. The reaction was quenched with water (30 mL) and the mixture was extracted with dichloromethane (3×50 mL). The combined organic layers were washed with brine (3×50 mL), dried over anhydrous Na₂SO₄, and concentrated under reduced pressure. The residue was purified by silica gel chromatography eluted with dichloromethane/methanol (25/1) to give 240 mg of methyl 5-[5-(2-{3-[(2-amino-5-bromophenyl)amino]-3-methylpyrrolidin-1-yl}ethoxy)-1-methylpyrazol-4-yl]-1-methyl-6-oxopyridine-3-carboxylate (INT-A11-6) as a brown solid (67%). LCMS: m/z (ES+), [M+H]⁺=559.05.

Step 7. Preparation of methyl 5-(5-{2-[3-(2-amino-6-bromo-1,3-benzodiazol-1-yl)-3-methylpyrrolidin-1-yl]ethoxy}-1-methylpyrazol-4-yl)-1-methyl-6-oxopyridine-3-carboxylate (INT-A11-7). To a stirred mixture of methyl 5-[5-(2-{3-[(2-amino-5-bromophenyl)amino]-3-methylpyrrolidin-1-yl}ethoxy)-1-methylpyrazol-4-yl]-1-methyl-6-oxopyridine-3-carboxylate (193 mg, 0.35 mmol, 1 equiv) in ethanol (10 mL) was added BrCN (40.19 mg, 0.38 mmol, 1.1 equiv) in portions at room temperature. The resulting mixture was stirred for 1 h at 40 °C. The mixture was cooled to room temperature. The reaction was quenched with water (30 mL) and the mixture was extracted with dichloromethane (3×30 mL). The combined organic layers were washed with brine (3×30 mL), dried over anhydrous Na₂SO₄, and concentrated under reduced pressure. The residue was purified by Prep-TLC eluted with dichloromethane/methanol (12/1) to give 140 mg of methyl 5-(5-{2-[3-(2-amino-6-bromo-1,3-benzodiazol-1-yl)-3-methylpyrrolidin-1-yl]ethoxy}-1-methylpyrazol-4-yl)-1-methyl-6-oxopyridine-3-carboxylate (INT-A11-7) as a brown solid (69%). LCMS: m/z (ES+), [M+H]⁺=586.00. ¹H NMR (400 MHz, DMSO-d₆) δ 1.24 (1H, s), 1.65 (3H, s), 2.35-2.48 (2H, m), 2.69-2.73 (1H, m), 2.80-2.90 (2H, m), 2.94-2.98 (1H, m), 3.18-3.22 (1H, m), 3.58 (3H, s), 3.75 (6H, d), 4.08-4.15 (2H, m), 6.52 (1H, s), 7.07 (1H, s), 7.43 (1H, s), 7.97 (1H, s), 8.13 (1H, d), 8.44 (1H, d).

Step 8. Preparation of 5-(5-{2-[3-(2-amino-6-bromo-1,3-benzodiazol-1-yl)-3-methylpyrrolidin-1-yl]ethoxy}-1-methylpyrazol-4-yl)-1-methyl-6-oxopyridine-3-carboxylic acid (INT-A11-8). A mixture of methyl 5-(5-{2-[3-(2-amino-6-bromo-1,3-benzodiazol-1-yl)-3-methylpyrrolidin-1-yl]ethoxy}-1-methylpyrazol-4-yl)-1-methyl-6-oxopyridine-3-carboxylate (170 mg, 0.29 mmol, 1 equiv) and LiOH·H₂O (50 mg, 1.16 mmol, 4 equiv) in tetrahydrofuran (4 mL) and water (1 mL) was stirred overnight at room temperature under nitrogen atmosphere and concentrated under reduced pressure. The residue was purified by prep-HPLC with XBridge Shield RP18 OBD column using water containing 10 mmol/L NH₄HCO₃ and 0.1% NH₃·H₂O and acetonitrile as mobile phase. Fractions containing the desired compound were evaporated to dryness to give 90 mg of 5-(5-{2-[3-(2-amino-6-bromo-1,3-benzodiazol-1-yl)-3-methylpyrrolidin-1-yl]ethoxy}-1-methylpyrazol-4-yl)-1-methyl-6-oxopyridine-3-carboxylic acid (INT-A11-8) as a white solid (54%). LCMS: m/z (ES+), [M+H]⁺=572.35.

Step 9. Preparation of 17-bromo-5,13,27-trimethyl-7-oxa-4,5,10,14,21,23,27-heptaazahexacyclo[23.3.1.1\hat{ }{10,13}.0\hat{ }{2,6}.0\hat{ }{14,22}.0\hat{ }{15,20}]triaconta-1(29),2(6),3,15,17,19,21,25-octaene-24,28-dione (INT-A11-9). A mixture of 5-(5-{2-[3-(2-amino-6-bromo-1,3-benzodiazol-1-yl)-3-methylpyrrolidin-1-yl]ethoxy}-1-methylpyrazol-4-yl)-1-methyl-6-oxopyridine-3-carboxylic acid (90 mg, 0.18 mmol, 1 equiv), N,N-diisopropylethylamine (45 mg, 0.35 mmol, 2 equiv) and N,N,N,N-tetramethyl-O-(7-azabenzotriazol-1-yl)uronium hexafluorophospate (133 mg, 0.35 mmol, 2 equiv) in dioxane (3 mL) was stirred overnight at room temperature under nitrogen atmosphere. The reaction was quenched with water (30 mL) and the mixture was extracted with dichloromethane (3×30 mL). The combined organic layers were washed with brine (3×30 mL), dried over anhydrous Na₂SO₄, and concentrated under reduced pressure. The residue was purified by Prep-TLC eluted with dichloromethane/methanol (12/1) to give 70 mg of 17-bromo-5,13,27-trimethyl-7-oxa-4,5,10,14,21,23,27-heptaazahexacyclo[23.3.1.1\hat{ }{10,13}.0\hat{ }{2,6}.0\hat{ }{14,22}.0\hat{ }{15,20}]triaconta-1(29),2(6),3,15,17,19,21,25-octaene-24,28-dione (INT-A11-9) as a white solid (72%). LCMS: m/z (ES+), [M+H]⁺=554.00.

Step 10. Preparation of 5,13,27-trimethyl-17-(morpholin-4-yl)-7-oxa-4,5,10,14,21,23,27-heptaazahexacyclo[23.3.1.1\hat{ }{10,13}.0\hat{ }{2,6}.0\hat{ }{14,22}.0\hat{ }{15,20}]triaconta-1(29),2(6),3,15,17,19,21,25-octaene-24,28-dione (EXAMPLE A11). To a stirred mixture of 17-bromo-5,13,27-trimethyl-7-oxa-4,5,10,14,21,23,27-heptaazahexacyclo[23.3.1.1\hat{ }{10,13}.0\hat{ }{2,6}.0\hat{ }{14,22}.0\hat{ }{15,20}]triaconta-1(29),2(6),3,15,17,19,21,25-octaene-24,28-dione (70 mg, 0.13 mmol, 1 equiv), BrettPhos (35 mg, 0.065 mmol, 0.5 equiv), and morpholine (35 mg, 0.40 mmol, 3.2 equiv) in dioxane (5 mL) was added LiHMDS (2.5 mL, 2.5 mmol, 19 equiv) dropwise at room temperature under nitrogen atmosphere. The mixture was stirred for 1 h at 60 °C under nitrogen atmosphere and then cooled to room temperature. The reaction was quenched with water (5 mL) and the mixture was extracted with dichloromethane (3×20 mL). The combined organic layers were washed with brine (3×20 mL), dried over anhydrous Na₂SO₄, and concentrated under reduced pressure. The residue was purified by Prep-TLC eluted with dichloromethane/methanol (10/1) to give 10.8 mg of 5,13,27-trimethyl-17-(morpholin-4-yl)-7-oxa-4,5,10,14,21,23,27-heptaazahexacyclo[23.3.1.1\hat{ }{10,13}.0\hat{ }{2,6}.0\hat{ }{14,22}.0\hat{ }{15,20}]triaconta-1(29),2(6),3,15,17,19,21,25-octaene-24,28-dione (EXAMPLE A11) as an off-white solid (15%). LCMS: m/z (ES+), [M+H]⁺=559.20. ¹H NMR (DMSO-dd, 400 MHz) δ 1.65 (3H, s), 2.65-2.84 (3H, m), 2.97-3.03 (2H, m), 3.01-3.24 (4H, m), 3.31-3.37 (1H, m), 3.62 (3H, s), 3.75 (3H, s), 3.77-3.83 (5H, m), 4.14-4.20 (1H, m), 4.35-4.40 (1H, m), 5.16 (1H, d), 6.93 (1H, dd), 6.98 (1H, s), 7.45 (1H, d), 8.22 (1H, d), 8.31 (1H, s), 8.89 (1H, d), 12.63 (1H, s).

### Example A12

### 15,21,27,27-Tetramethyl-5-(morpholin-4-yl)-23-oxa-2,9,11,15,20,21,26-heptaazahexacyclo[24.3.1.1\hat{ }{13,17}.0\hat{ }{2,10}.0\hat{ }{3,8}.0\hat{ }{18,22}]hentriaconta-3,5,7,9,13,17(31),18(22),19-octaene-12,16-dione

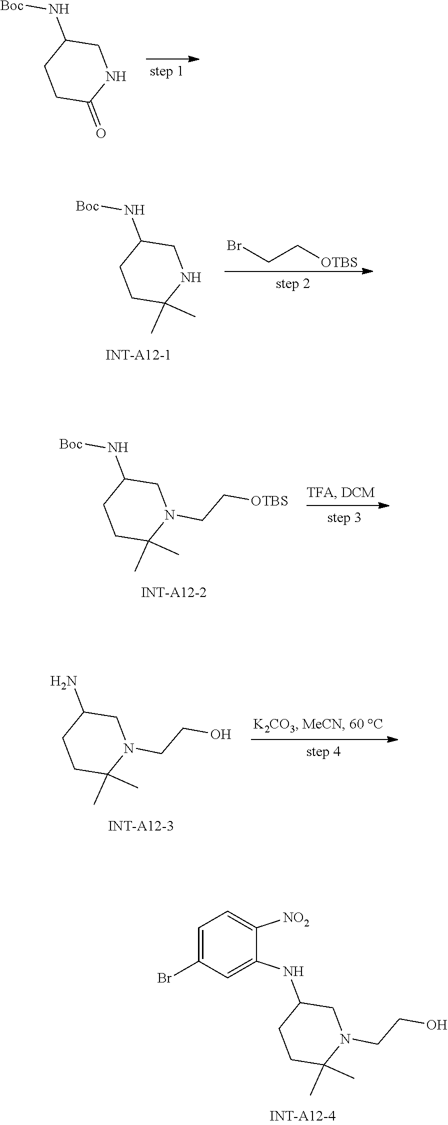 [SMILES: CC(C)(C)OC(=O)NC1CCC(=O)NC1 ; CC1(C)CCC(NC(=O)OC(C)(C)C)CN1 ; CC(C)(C)[Si](C)(C)OCCBr ; CC(C)(C)OC(=O)NC1CCC(C)(C)N(CCO[Si](C)(C)C(C)(C)C)C1 ; CC1(C)CCC(N)CN1CCO ; CC1(C)CCC(Nc2cc(Br)ccc2[N+](=O)[O-])CN1CCO]

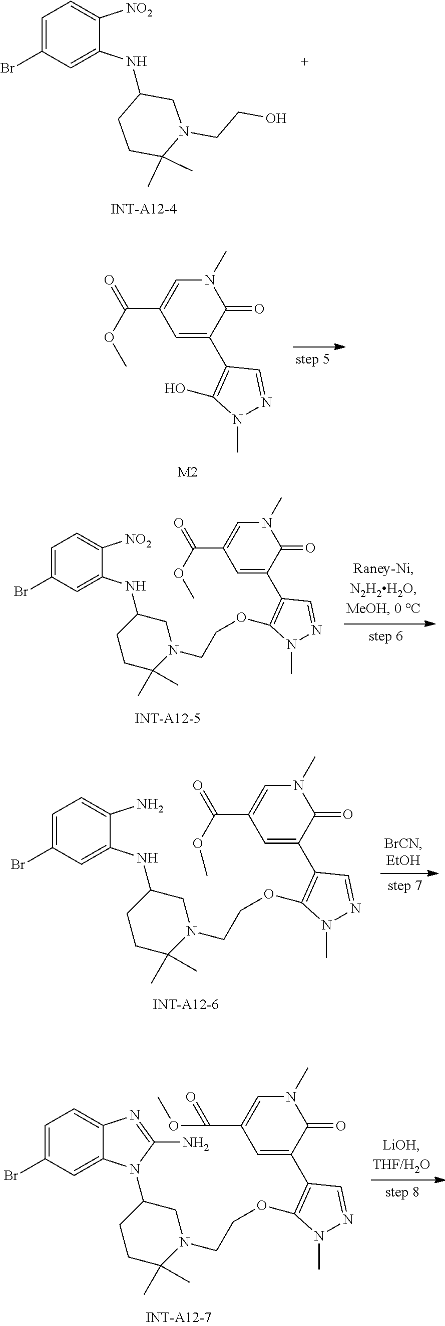 [SMILES: CC1(C)CCC(Nc2cc(Br)ccc2[N+](=O)[O-])CN1CCO ; COC(=O)c1cc(-c2cnn(C)c2O)c(=O)n(C)c1 ; "M2" ; COC(=O)c1cc(-c2cnn(C)c2OCCN2CC(Nc3cc(Br)ccc3[N+](=O)[O-])CCC2(C)C)c(=O)n(C)c1 ; COC(=O)c1cc(-c2cnn(C)c2OCCN2CC(Nc3cc(Br)ccc3N)CCC2(C)C)c(=O)n(C)c1 ; COC(=O)c1cc(-c2cnn(C)c2OCCN2CC(n3c(N)nc4ccc(Br)cc43)CCC2(C)C)c(=O)n(C)c1]

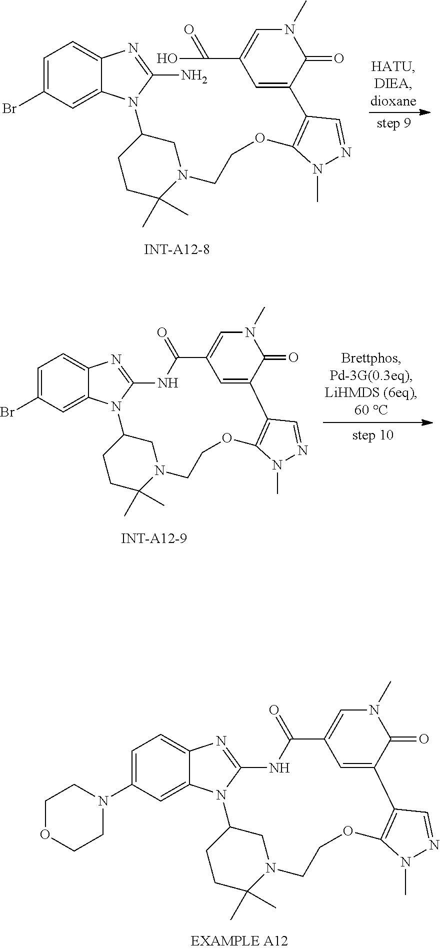 [SMILES: Cn1ncc(-c2cc(C(=O)O)cn(C)c2=O)c1OCCN1CC(n2c(N)nc3ccc(Br)cc32)CCC1(C)C ; Cn1ncc2c1OCCN1CC(CCC1(C)C)n1c(nc3ccc(Br)cc31)NC(=O)c1cc-2c(=O)n(C)c1 ; Cn1ncc2c1OCCN1CC(CCC1(C)C)n1c(nc3ccc(N4CCOCC4)cc31)NC(=O)c1cc-2c(=O)n(C)c1]

Step 1. Preparation of tert-butyl N-(6,6-dimethylpiperidin-3-yl)carbamate (INT-A 12-1). A solution of tert-butyl N-(6-oxopiperidin-3-yl)carbamate (4 g, 18.66 mmol, 1 equiv) in THF (50 mL) was treated with tetrachlorozirconium (10.44 g, 44.80 mmol, 2.4 equiv) for 0.5 h at 10 °C under nitrogen atmosphere followed by the addition of CH₃MgBr (28.94 g, 242.68 mmol, 13 equiv) dropwise at 0 °C. The resulting mixture was stirred for 16 h at room temperature under nitrogen atmosphere. The reaction was quenched with water (500 mL) and the mixture was extracted with ethyl acetate (3×400 mL). The combined organic layers were washed with brine (3×400 mL), dried over anhydrous Na₂SO₄, and concentrated under reduced pressure. The residue was purified by silica gel chromatography eluted with dichloromethane/methanol (30/1) to give 3.74 g of tert-butyl N-(6,6-dimethylpiperidin-3-yl)carbamate (INT-A12-1) as a yellow solid (87%). LCMS: m/z (ESI), [M+H]⁺=229.05.

Step 2. Preparation of tert-butyl N-(1-{2-[(tert-butyldimethylsilyl)oxy]ethyl}-6,6-dimethylpiperidin-3-yl)carbamate (INT-A12-2). To a stirred mixture of tert-butyl N-(6,6-dimethylpiperidin-3-yl)carbamate (INT-A12-1, 234 mg, 1.02 mmol, 1 equiv) and K₂CO₃ (354 mg, 2.56 mmol, 2.5 equiv) in DMSO (10 mL) was added (2-bromoethoxy)(tert-butyl)dimethylsilane (294 mg, 1.23 mmol, 1.2 equiv) dropwise at room temperature. The reaction mixture was stirred at 60 °C for 3 h. After being cooled to room temperature, the reaction mixture was treated with water (100 mL) and extracted with ethyl acetate (3×100 mL). The combined organic layers were washed with brine (3×100 mL), dried over anhydrous Na₂SO₄, and concentrated under reduced pressure. The residue was purified by silica gel chromatography eluted with petroleum ether/ethyl acetate (10/1) to give 128 mg of tert-butyl N-(1-{2-[(tert-butyldimethylsilyl)oxy]ethyl}-6,6-dimethylpiperidin-3-yl)carbamate (INT-A12-2) as a yellow oil (32%). LCMS: m/z (ESI), [M+H]⁺=387.15.

Step 3. Preparation of 2-(5-amino-2,2-dimethylpiperidin-1-yl)ethanol (INT-A12-3). A solution of tert-butyl N-(1-{2-[(tert-butyldimethylsilyl)oxy]ethyl}-6,6-dimethylpiperidin-3-yl)carbamate (INT-A12-2, 20 g, 50 mmol, 1 equiv) and trifluoroacetic acid (60 mL) in dichloromethane (200 mL) was stirred for 2 h at room temperature. The resulting mixture was concentrated under reduced pressure. The crude product of 2-(5-amino-2,2-dimethylpiperidin-1-yl)ethanol (INT-A12-3) as a yellow oil was used in the next step directly without further purification. LCMS: m/z (ESI), [M+H]⁺=173.00.

Step 4. Preparation of 2-{5-[(5-bromo-2-nitrophenyl)amino]-2,2-dimethylpiperidin-1-yl}ethanol (INT-A12-4). A mixture of 2-(5-amino-2,2-dimethylpiperidin-1-yl)ethanol (crude, INT-A12-3), 4-bromo-2-fluoro-1-nitrobenzene (10.33 g, 60 mmol, 1.2 equiv) and K₂CO₃ (20.67 g, 52.24 mmol, 3 equiv) in acetonitrile (150 mL) was stirred for 2 h at 60 °C. After being cooled to room temperature, the reaction was quenched with water (800 mL) and the mixture was extracted with ethyl acetate (3×600 mL). The combined organic layers were washed with brine (3×600 mL), dried over anhydrous Na₂SO₄, and concentrated under reduced pressure. The residue was purified by silica gel column chromatography eluted with petroleum ether/ethyl acetate (5/1) to give 9.3 g of 2-{5-[(5-bromo-2-nitrophenyl)amino]-2,2-dimethylpiperidin-1-yl}ethanol (INT-A12-4) as a yellow solid (410%). LCMS: m/z (ESI), [M+H]⁺=374.05. ¹H NMR (DMSO-d₆, 400 MHz) δ 0.99 (3H, s), 1.05 (3H, s), 1.26-1.46 (2H, m), 1.63-1.70 (2H, m), 2.17-2.25 (1H, m), 2.50-2.60 (3H, m), 3.42 (2H, q), 3.86-3.92 (1H, m), 4.32 (1H, t), 6.76-7.82 (1H, m), 7.27 (1H, d), 8.00 (1H, d), 8.60 (1H, d).

Step 5. Preparation of methyl 5-[5-(2-{5-[(5-bromo-2-nitrophenyl)amino]-2,2-dimethylpiperidin-1-yl}ethoxy)-1-methylpyrazol-4-yl]-1-methyl-6-oxopyridine-3-carboxylate (INT-A12-5). A mixture of 2-{5-[(5-bromo-2-nitrophenyl)amino]-2,2-dimethylpiperidin-1-yl}ethanol (INT-A12-4, 350 mg, 0.94 mmol, 1 equiv), methyl 5-(5-hydroxy-1-methylpyrazol-4-yl)-1-methyl-6-oxopyridine-3-carboxylate (M2, 247 mg, 0.94 mmol, 1 equiv) and 2-(tributyl-1\hat{ }[5]-phosphanylidene)acetonitrile (1.13 g, 4.70 mmol, 5 equiv) in toluene (8 mL) was stirred for 3 h at 100 °C. After being cooled to room temperature, the reaction was quenched with water (100 mL) and the mixture was extracted with ethyl acetate (3×100 mL). The combined organic layers were washed with brine (3×100 mL), dried over anhydrous Na₂SO₄, and concentrated under reduced pressure. The residue was purified by reverse flash chromatography eluted with 10%-50% acetonitrile in water to give 350 mg of methyl 5-[5-(2-{5-[(5-bromo-2-nitrophenyl)amino]-2,2-dimethylpiperidin-1-yl}ethoxy)-1-methylpyrazol-4-yl]-1-methyl-6-oxopyridine-3-carboxylate (INT-A12-5) as a yellow solid (60%). LCMS: m/z (EST), [M+H]⁺=617.20.

Step 6. Preparation of methyl 5-[5-(2-{5-[(2-amino-5-bromophenyl)amino]-2,2-dimethylpiperidin-1-yl}ethoxy)-1-methylpyrazol-4-yl]-1-methyl-6-oxopyridine-3-carboxylate (INT-A12-6). A mixture of methyl 5-[5-(2-{5-[(5-bromo-2-nitrophenyl)amino]-2,2-dimethylpiperidin-1-yl}ethoxy)-1-methylpyrazol-4-yl]-1-methyl-6-oxopyridine-3-carboxylate (INT-A12-5, 400 mg, 0.64 mmol, 1 equiv), raney-Ni (5 mg, 0.15 mmol, 0.5 equiv) and hydrazine (62 mg, 1.94 mmol, 3 equiv) in methanol (10 mL) was stirred for 2 h at room temperature under nitrogen atmosphere. The reaction was quenched with water (100 mL), and the mixture was extracted with ethyl acetate (3×100 mL). The combined organic layers were washed with brine (3×100 mL), dried over anhydrous Na₂SO₄, and concentrated under reduced pressure. The residue was purified by prep-TLC eluted with dichloromethane/methanol (20/1) to give 350 mg of methyl 5-[5-(2-{5-[(2-amino-5-bromophenyl)amino]-2,2-dimethylpiperidin-1-yl}ethoxy)-1-methylpyrazol-4-yl]-1-methyl-6-oxopyridine-3-carboxylate (INT-A12-6) as a yellow solid (91%). LCMS: m/z (ESI), [M+H]⁺=589.35.

Step 7. Preparation of methyl 5-(5-{2-[5-(2-amino-6-bromo-1,3-benzodiazol-1-yl)-2,2-dimethylpiperidin-1-yl]ethoxy}-1-methylpyrazol-4-yl)-1-methyl-6-oxopyridine-3-carboxylate (INT-A12-7). A mixture of methyl 5-[5-(2-{5-[(2-amino-5-bromophenyl)amino]-2,2-dimethylpiperidin-1-yl}ethoxy)-1-methylpyrazol-4-yl]-1-methyl-6-oxopyridine-3-carboxylate (INT-A12-6, 350 mg, 0.59 mmol, 1 equiv) and cyanogen bromide (69 mg, 0.65 mmol, 1.1 equiv) in EtOH (10 mL) was stirred for 3 h at room temperature under nitrogen atmosphere. The reaction mixture was treated with sat. NaHCO₃ solution (100 mL) and extracted with ethyl acetate (3×100 mL). The combined organic layers were dried over anhydrous Na₂SO₄ and concentrated under reduced pressure. The residue was purified by prep-TLC eluted with dichloromethane/methanol (20/1) to give 350 mg of methyl 5-(5-{2-[5-(2-amino-6-bromo-1,3-benzodiazol-1-yl)-2,2-dimethylpiperidin-1-yl]ethoxy}-1-methylpyrazol-4-yl)-1-methyl-6-oxopyridine-3-carboxylate (INT-A12-7) as a yellow solid (95%). LCMS: m/z (EST), [M+H]⁺=614.05.

Step 8. Preparation of 5-(5-{2-[5-(2-amino-6-bromo-1,3-benzodiazol-1-yl)-2,2-dimethylpiperidin-1-yl]ethoxy}-1-methylpyrazol-4-yl)-1-methyl-6-oxopyridine-3-carboxylic acid (INT-A12-8). To a stirred solution of methyl 5-(5-{2-[5-(2-amino-6-bromo-1,3-benzodiazol-1-yl)-2,2-dimethylpiperidin-1-yl]ethoxy}-1-methylpyrazol-4-yl)-1-methyl-6-oxopyridine-3-carboxylate (INT-A12-7, 240 mg, 0.39 mmol, 1 equiv) in tetrahydrofuran (4 mL) was added lithium hydroxide (28 mg, 1.17 mmol, 3 equiv) in H₂O (1 mL). The resulting mixture was stirred for 2 h at room temperature. The reaction mixture was concentrated under reduced pressure and the residue was purified by reverse flash chromatography eluted with 10% to 40% acetonitrile in water to give 190 mg of 5-(5-{2-[5-(2-amino-6-bromo-1,3-benzodiazol-1-yl)-2,2-dimethylpiperidin-1-yl]ethoxy}-1-methylpyrazol-4-yl)-1-methyl-6-oxopyridine-3-carboxylic acid (INT-A12-8) as a white solid (81%). LCMS: m/z (ESI), [M+H]⁺=598.30.

Step 9. Preparation of 5-bromo-15,21,27,27-tetramethyl-23-oxa-2,9,11,15,20,21,26-heptaazahexacyclo[24.3.1.1\hat{ }{13,17}.0\hat{ }{2,10}.0\hat{ }{3,8}.0\hat{ }{18,22}]hentriaconta-3,5,7,9,13,17(31),18(22),19-octaene-12,16-dione (INT-A12-9). A solution of 5-(5-{2-[5-(2-amino-6-bromo-1,3-benzodiazol-1-yl)-2,2-dimethylpiperidin-1-yl]ethoxy}-1-methylpyrazol-4-yl)-1-methyl-6-oxopyridine-3-carboxylic acid (INT-A12-8, 190 mg, 0.31 mmol, 1 equiv), N,N-Diisopropylethylamine (82 mg, 0.63 mmol, 2 equiv) and N,N,N,N-tetramethyl-O-(7-azabenzotriazol-1-yl)uronium hexafluorophospate (181 mg, 0.47 mmol, 1.5 equiv) in dioxane (5 mL) was stirred for 2 h at room temperature under nitrogen atmosphere. The reaction was quenched with water (100 mL) and the mixture was extracted with ethyl acetate (3×100 mL). The combined organic layers were washed with brine (3×100 mL), dried over anhydrous Na₂SO₄, and concentrated under reduced pressure. The residue was purified by prep-TLC eluted with dichloromethane/methanol (20/1) to give to give 100 mg of 5-bromo-15,21,27,27-tetramethyl-23-oxa-2,9,11,15,20,21,26-heptaazahexacyclo[24.3.1.1\hat{ }{13,17}.0\hat{ }{2,10}.0\hat{ }{3,8}.0\hat{ }{18,22}]hentriaconta-3,5,7,9,13,17(31),18(22),19-octaene-12,16-dione (INT-A12-9) as a white solid (27%). LCMS: m/z (EST), [M+H]⁺=580.35.

Step 10. Preparation of 15,21,27,27-tetramethyl-5-(morpholin-4-yl)-23-oxa-2,9,11,15,20,21,26-heptaazahexacyclo[24.3.1.1\hat{ }{13,17}.0\hat{ }{2,10}.0\hat{ }{3,8}.0\hat{ }{18,22}]hentriaconta-3,5,7,9,13,17(31),18(22),19-octaene-12,16-dione (EXAMPLE A12). To a stirred mixture of 5-bromo-15,21,27,27-tetramethyl-23-oxa-2,9,11,15,20,21,26-heptaazahexacyclo[24.3.1.1\hat{ }{13,17}.0\hat{ }{2,10}.0\hat{ }{3,8}.0\hat{ }{18,22}]hentriaconta-3,5,7,9,13,17(31),18(22),19-octaene-12,16-dione (INT-A12-9, 60 mg, 0.10 mmol, 1 equiv), morpholine (36 mg, 0.41 mmol, 4 equiv), and BrettPhos Pd G₃ (46 mg, 0.05 mmol, 0.5 equiv) in dioxane (3 mL) was added LiHMDS (0.5 mL, 0.51 mmol, 5 equiv) dropwise at room temperature under nitrogen atmosphere. The reaction mixture was stirred for 1 h at 60 °C under nitrogen atmosphere. After being cooled to room temperature, the reaction was quenched with sat. NH₄Cl solution (50 mL) and the mixture was extracted with dichloromethane (3×50 mL). The combined organic layers were washed with brine (3×50 mL), dried over anhydrous Na₂SO₄, and concentrated under reduced pressure. The residue was purified by prep-TLC eluted with dichloromethane/methanol (18/1). The product was further purified by prep-HPLC with XBridge Shield RP18 OBD column using water containing 10 mmol/L NH₄HCO and 0.1% NH₃·H₂O and acetonitrile as mobile phase. Fractions containing the desired compound were evaporated to dryness to give 8.9 mg of 515,21,27,27-tetramethyl-5-(morpholin-4-yl)-23-oxa-2,9,11,15,20,21,26-heptaazahexacyclo[24.3.1.1\hat{ }{13,17}.0\hat{ }{2,10}.0\hat{ }{3,8}.0\hat{ }{18,22}]hentriaconta-3,5,7,9,13,17(31),18(22),19-octaene-12,16-dione (EXAMPLE A12) as a white solid (14%). LCMS: m/z (ESI), [M+H]⁺=587.40. ¹H NMR (DMSO-d₆, 400 MHz) δ 1.12 (3H, s), 1.20-1.28 (1H, m), 1.30 (3H, s), 1.61-1.85 (2H, m), 2.17-2.29 (2H, m), 2.42 (1H, t), 3.05-3.18 (4H, m), 3.39-3.60 (3H, m), 3.62 (3H, s), 3.69 (3H, s), 3.78 (4H, t), 4.48-4.69 (2H, m), 6.87-6.94 (1H, m), 7.18 (1H, s), 7.43 (1H, d), 8.29 (1H, s), 8.37 (1H, s), 8.80 (1H, d), 11.81-12.75 (1H, m)

### Example A13

### 1,15,21-Trimethyl-5-(morpholin-4-yl)-23-oxa-2,9,11,15,20,21,26heptaazahexacyclo[24.4.1.1\hat{ }{13,17}.0\hat{ }{2,10}.0\hat{ }{3,8}.0\hat{ }{18,22}]dotriac onta-3,5,7,9,13,17(32),18(22),19-octaene-12,16-dione

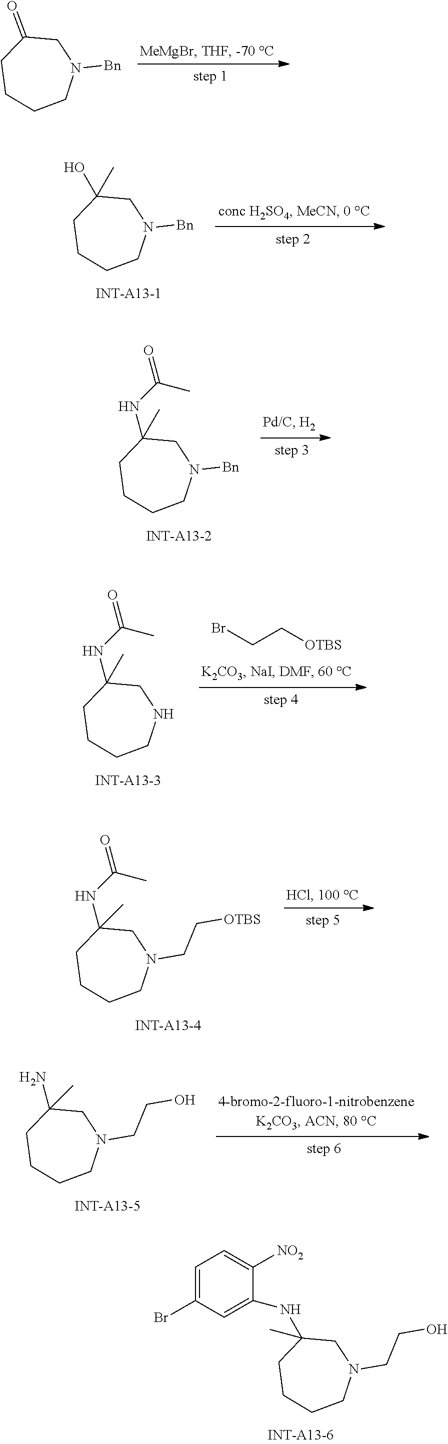

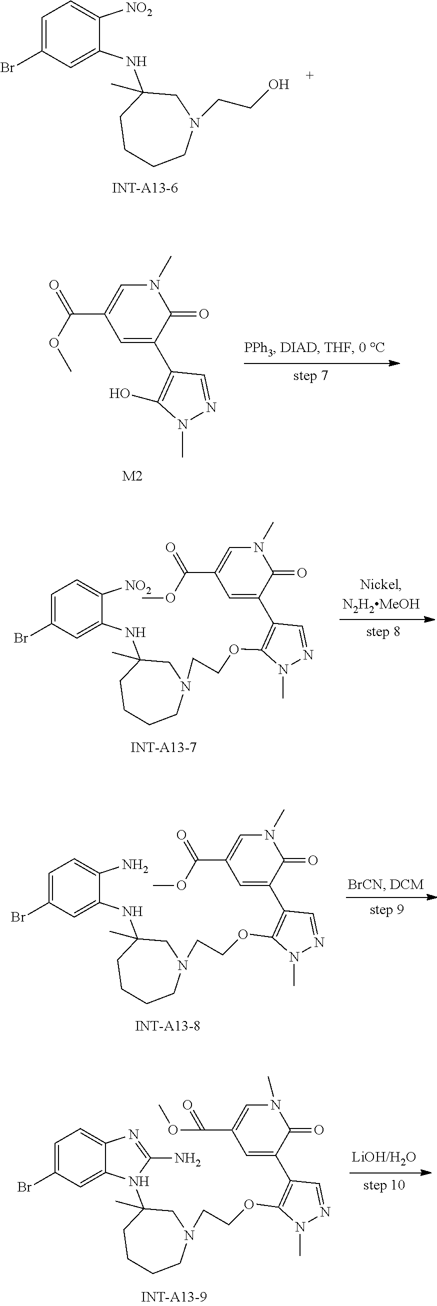 [SMILES: CC1(Nc2cc(Br)ccc2[N+](=O)[O-])CCCCN(CCO)C1 ; COC(=O)c1cc(-c2cnn(C)c2O)c(=O)n(C)c1 ; "M2" ; COC(=O)c1cc(-c2cnn(C)c2OCCN2CCCCC(C)(Nc3cc(Br)ccc3[N+](=O)[O-])C2)c(=O)n(C)c1 ; COC(=O)c1cc(-c2cnn(C)c2OCCN2CCCCC(C)(Nc3cc(Br)ccc3N)C2)c(=O)n(C)c1 ; COC(=O)c1cc(-c2cnn(C)c2OCCN2CCCCC(C)(n3c(N)nc4ccc(Br)cc43)C2)c(=O)n(C)c1]

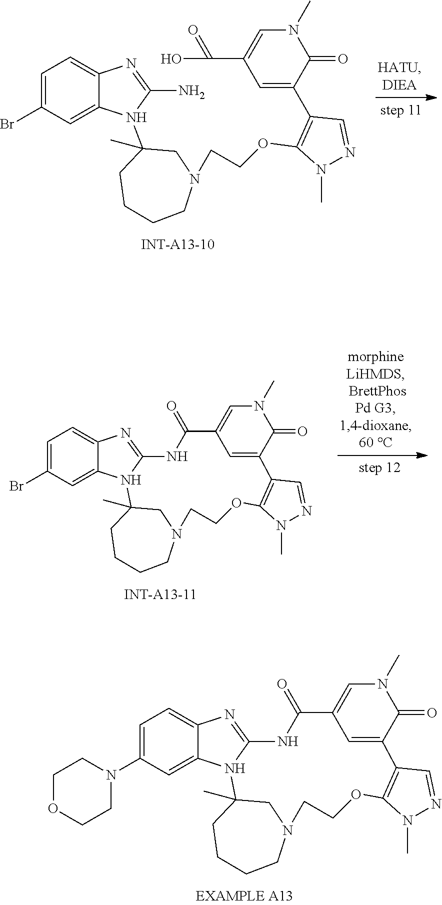 [SMILES: Cn1ncc(-c2cc(C(=O)O)cn(C)c2=O)c1OCCN1CCCCC(C)(n2c(N)nc3ccc(Br)cc32)C1 ; Cn1ncc2c1OCCN1CCCCC(C)(C1)n1c(nc3ccc(Br)cc31)NC(=O)c1cc-2c(=O)n(C)c1 ; Cn1ncc2c1OCCN1CCCCC(C)(C1)n1c(nc3ccc(N4CCOCC4)cc31)NC(=O)c1cc-2c(=O)n(C)c1]

Step 1. Preparation of 1-benzyl-3-methylazepan-3-ol (INT-A13-1). To a stirred mixture of 1-benzylazepan-3-one (3.0 g, 14.75 mmol, 1 equiv) in tetrahydrofuran (30 mL) was added MeMgBr (10.5 g, 88.54 mmol, 6 equiv) dropwise at −78 °C under nitrogen atmosphere. The mixture was stirred for 2 h at −60 °C. The reaction was quenched with sat. NH₄Cl (aq.) solution (300 mL) and the mixture was extracted with dichloromethane (3×300 mL). The combined organic layers were washed with brine (3×300 mL), dried over anhydrous Na₂SO₄, and concentrated under reduced pressure to give 3.1 g of 1-benzyl-3-methylazepan-3-ol (INT-A13-1) as a brown oil. This material was used for the next reaction without purification. LCMS: m/z (ESI), [M+H]⁺=220.05.

Step 2. Preparation of N-(1-benzyl-3-methylazepan-3-yl)acetamide (INT-A13-2). To a stirred mixture of 1-benzyl-3-methylazepan-3-ol (3.1 g, ~14 mmol, ~1 equiv) in acetonitrile (30 mL) was added fuming H₂SO₄ (2.5 g, 14.13 mmol, 1 equiv) dropwise at 0 °C under nitrogen atmosphere. The mixture was stirred for 2 h. The reaction was quenched with water (300 mL) at room temperature. The mixture was adjusted to pH 9 with K₂CO₃ and extracted with dichloromethane (3×300 mL). The combined organic layers were washed with brine (3×300 mL), dried over anhydrous Na₂SO₄, and concentrated under reduced pressure to give 1.1 g of N-(1-benzyl-3-methylazepan-3-yl)acetamide (INT-A13-2) as a brown solid. ¹H NMR (400 MHz, DMSO-d₆) δ 1.18 (3H, s), 1.35-1.67 (5H, m), 1.72 (3H, s), 1.90-2.00 (1H, m), 2.61 (1H, d), 2.87 (1H, d), 3.61 (2H, d), 7.20-7.37 (5H, m).

Step 3. Preparation of N-(3-methylazepan-3-yl)acetamide (INT-A13-3). A mixture of N-(1-benzyl-3-methylazepan-3-yl)acetamide (INT-A13-2, 1.1 g, 4.22 mmol, 1 equiv) and Pd/C (0.4 g, 4.22 mmol, 1 equiv) in methanol (10 mL) was stirred overnight at room temperature under nitrogen atmosphere. The mixture was filtered. The filter cake was washed with methanol (3×100 mL). The resulting solution was concentrated under reduced pressure. The crude product was used in the next step directly without further purification. ¹H NMR (400 MHz, DMSO-d₆) δ 1.20 (3H, s), 1.32-1.58 (5H, m), 1.78 (3H, s), 1.90-2.10 (1H, m), 2.61 (1H, d), 2.65-2.77 (2H, m), 2.85 (1H, d), 7.23 (1H, s).

Step 4. Preparation of N-(1-(2-((tert-butyldimethylsilyl)oxy)ethyl)-3-methylazepan-3-yl)acetamide (INT-A13-4). To a stirred mixture of N-(3-methylazepan-3-yl)acetamide (400 mg, ~2.3 mmol, 1 equiv) and (2-bromoethoxy)(tert-butyl)dimethylsilane (1124 mg, 4.69 mmol, 2 equiv) in N,N-dimethylformamide (8 mL) were added K₂CO₃ (649.4 mg, 4.69 mmol, 2 equiv) and NaI (704 mg, 4.69 mmol, 2 equiv). The mixture was stirred for 2 h at 60 °C and then cooled to room temperature. The reaction was quenched with water (100 mL) and the mixture was extracted with ethyl acetate (3×100 mL). The combined organic layers were washed with brine (3×100 mL), dried over anhydrous Na₂SO₄, and concentrated under reduced pressure. The residue was purified by C18 FLASH using water and acetonitrile as mobile phase. Fractions containing the desired compound were evaporated to dryness to give 750 mg of N-(1-{2-[(tert-butyldimethylsilyl)oxy]ethyl}-3-methylazepan-3-yl)acetamide (INT-A13-4) as a brown oil (96%). ¹H NMR (400 MHz, DMSO-d₆) δ 0.01 (6H, s), 0.83 (9H, s), 1.19 (3H, s), 1.28-1.62 (6H, m), 1.73 (3H, s), 1.86 (1H, d), 2.51-2.64 (5H, m), 2.72-2.80 (1H, m), 3.55-3.63 (2H, m).

Step 5. Preparation of 2-(3-amino-3-methylazepan-1-yl)ethanol (INT-A13-5). A mixture of N-(1-{2-[(tert-butyldimethylsilyl)oxy]ethyl}-3-methylazepan-3-yl)acetamide (1.4 g, 4.26 mmol, 1 equiv) and HCl (6 M) (140 mL, 460.77 mmol, 108 equiv) was stirred overnight at 100 °C under nitrogen atmosphere. The resulting mixture was concentrated under reduced pressure to give 500 mg of 2-(3-amino-3-methylazepan-1-yl)ethanol (INT-A13-5) as a brown oil. ¹H NMR (400 MHz, DMSO-d₆) δ 1.11-1.39 (3H, m), 1.48 (2H, d), 1.70 (1H, d), 1.76-2.08 (6H, m), 2.19 (1H, t), 3.18 (2H, t), 3.34-3.52 (1H, m), 3.52-3.95 (6H, m), 8.64 (2H, s).

Step 6. Preparation of 2-(3-((5-bromo-2-nitrophenyl)amino)-3-methylazepan-1-yl)ethanol (INT-A13-6). To a stirred mixture of 2-(3-amino-3-methylazepan-1-yl)ethanol (460 mg, 2.67 mmol, 1 equiv) and 4-bromo-2-fluoro-1-nitrobenzene (1174 mg, 5.34 mmol, 2 equiv) in acetonitrile (5 mL) was added K₂CO₃ (738 mg, 5.34 mmol, 2 equiv) in portions. The mixture was stirred overnight at 80 °C and then cooled to room temperature. The reaction was quenched with water (200 mL) and the mixture was extracted with ethyl acetate (3×200 mL). The combined organic layers were washed with brine (3×200 mL), dried over anhydrous Na₂SO₄, and concentrated under reduced pressure. The residue was purified by silica gel chromatography eluted with petroleum ether/ethyl acetate (1/10) to give 530 mg of 2-{3-[(5-bromo-2-nitrophenyl)amino]-3-methylazepan-1-yl}ethanol (INT-A13-6) as a yellow oil (53%). ¹H NMR (400 MHz, DMSO-d₆) δ 1.31-1.63 (7H, m), 1.64-1.77 (1H, m), 2.21 (1H, d), 2.58-2.86 (6H, m), 3.60 (2H, d), 4.44 (1H, t), 6.81 (1H, d), 7.41 (1H, d), 8.01 (1H, d), 9.07 (1H, s).

Step 7. Preparation of methyl 5-[5-(2-{3-[(5-bromo-2-nitrophenyl)amino]-3-methylazepan-1-yl}ethoxy)-1-methylpyrazol-4-yl]-1-methyl-6-oxopyridine-3-carboxylate (INT-A13-7). To a stirred mixture of methyl 5-(5-hydroxy-1-methylpyrazol-4-yl)-1-methyl-6oxopyridine-3-carboxylate (M2, 405 mg, 1.53 mmol, 1 equiv) and triphenylphosphine (1210 mg, 4.614 mmol, 3 equiv) in tetrahydrofuran (5 mL) were added diisopropyl azodicarboxylate (933 mg, 4.61 mmol, 3 equiv) and 2-{3-[(5-bromo-2-nitrophenyl)amino]-3-methylazepan-1-yl}ethanol (629 mg, 1.69 mmol, 1.1 equiv) at 0 °C under nitrogen atmosphere. The mixture was stirred for 2 h at room temperature. The reaction was quenched with water (100 mL) and the mixture was extracted with ethyl acetate (3×100 mL). The combined organic layers were washed with brine (3×100 mL), dried over anhydrous Na₂SO₄, and concentrated under reduced pressure. The residue was purified by Prep-TLC eluted with CH₂Cl₂/methanol (10/1) to give 550 mg of methyl 5-[5-(2-{3-[(5-bromo-2-nitrophenyl)amino]-3-methylazepan-1-yl}ethoxy)-1-methylpyrazol-4-yl]-1-methyl-6-oxopyridine-3-carboxylate (INT-A13-7) as a yellow oil (57%). ¹H NMR (400 MHz, DMSO-d₆) δ 1.37 (3H, s), 1.44-1.50 (1H, m), 1.65-1.75 (1H, m), 2.17-2.25 (1H, m), 2.63-2.77 (2H, m), 2.83 (1H, s), 2.91 (1H, d), 3.10 (1H, d), 3.10-3.20 (1H, m), 3.33 (2H, s), 3.71 (6H, d), 4.06-4.22 (2H, m), 5.76 (1H, s), 6.80 (1H, d), 7.20-7.30 (2H, m), 7.33-7.50 (5H, m), 7.94 (1H, s), 7.98 (1H, d), 8.07 (1H, d), 8.40 (1H, d), 9.01 (1H, s).

Step 8. Preparation of methyl 5-(5-(2-(3-((2-amino-5-bromophenyl)amino)-3-methylazepan-1-yl)ethoxy)-1-methyl-1H-pyrazol-4-yl)-1-methyl-6-oxo-1,6-dihydropyridine-3-carboxylate (INT-A13-8). To a stirred mixture of methyl 5-[5-(2-{3-[(5-bromo-2-nitrophenyl)amino]-3-methylazepan-1-yl}ethoxy)-1-methylpyrazol-4-yl]-1-methyl-6-oxopyridine-3-carboxylate (540 mg, 0.87 mmol, 1 equiv), Raney Ni (74 mg, 0.87 mmol, 1 equiv) and NH₂NH₂H₂O (44 mg, 0.88 mmol, 1 equiv) in methanol (2 mL) was stirred for 1 h at 0 °C under nitrogen atmosphere. The resulting mixture was filtered. The filter cake was washed with methanol (50 mL). The resulting solution was concentrated under reduced pressure to give 300 mg of 5-[5-(2-{3-[(2-amino-5-bromophenyl)amino]-3-methylazepan-1-yl}ethoxy)-1-methylpyrazol-4-yl]-1-methyl-6-oxopyridine-3-carboxylate (INT-A13-8) as a brown oil. This material was used for the next reaction without purification. LCMS: m/z (ESI), [M+H]+=589.20.

Step 9. Preparation of methyl 5-(5-{2-[3-(2-amino-6-bromo-1,3-benzodiazol-1-yl)-3-methylazepan-1-yl]ethoxy}-1-methylpyrazol-4-yl)-1-methyl-6-oxopyridine-3-carboxylate (INT-A13-9). A mixture of methyl 5-[5-(2-{3-[(2-amino-5-bromophenyl)amino]-3-methylazepan-1-yl}ethoxy)-1-methylpyrazol-4-yl]-1-methyl-6-oxopyridine-3-carboxylate (280 mg, 0.47 mmol, 1 equiv) and BrCN (50 mg, 0.47 mmol, 1 equiv) in EtOH (4 mL) was stirred for 2 h at 60 0 °C under nitrogen atmosphere. The mixture was cooled to room temperature and concentrated under reduced pressure. The residue was purified by Prep-TLC eluted with CH₂Cl₂/methanol (10/1) to give 210 mg of methyl 5-(5-{2-[3-(2-amino-6-bromo-1,3-benzodiazol-1-yl)-3-methylazepan-1-yl]ethoxy}-1-methylpyrazol-4-yl)-1-methyl-6-oxopyridine-3-carboxylate (INT-A13-9) as a brown oil (71%). ¹H NMR (400 MHz, DMSO-d₆) δ 1.19-1.23 (1H, m), 1.43-1.49 (1H, m), 1.60-1.64 (2H, m), 1.73-1.90 (5H, m), 2.65-2.81 (1H, m), 2.83-2.90 (1H, m), 2.90-3.05 (2H, m), 3.17 (3H, d), 3.55 (3H, s), 3.66 (3H, s), 3.74 (3H, s), 4.00-4.18 (3H, m), 6.95-7.11 (2H, m), 7.54 (1H, d), 7.70 (2H, s), 7.94 (1H, s), 8.06 (1H, d), 8.37 (1H, d).

Step 10. Preparation of 5-(5-{2-[3-(2-amino-6-bromo-1,3-benzodiazol-1-yl)-3-methylazepan-1-yl]ethoxy}-1-methylpyrazol-4-yl)-1-methyl-6-oxopyridine-3-carboxylic acid (INT-A13-10). A mixture of methyl 5-(5-{2-[3-(2-amino-6-bromo-1,3-benzodiazol-1-yl)-3-methylazepan-1-yl]ethoxy}-1-methylpyrazol-4-yl)-1-methyl-6-oxopyridine-3-carboxylate (200 mg, 0.32 mmol, 1 equiv) and LiOH·H₂O (27 mg, 0.65 mmol, 2 equiv) in tetrahydrofuran/H₂O (4 ml/1 mL) was stirred for 2 h at room temperature under nitrogen atmosphere. The mixture was concentrated under reduced pressure. The residue was purified by C18 FLASH using water and acetonitrile as mobile phase. Fractions containing the desired compound were evaporated to dryness to give 150 mg of 5-(5-{2-[3-(2-amino-6-bromo-1,3-benzodiazol-1-yl)-3-methylazepan-1-yl]ethoxy}-1-methylpyrazol-4-yl)-1-methyl-6-oxopyridine-3-carboxylic acid (INT-A13-10) as a brown solid (76%). ¹H NMR (400 MHz, DMSO-d₆) δ 1.43-1.68 (2H, m), 1.81 (3H, d), 2.70-2.74 (1H, m), 2.82-3.02 (2H, m), 3.06-3.12 (1H, m), 3.38-3.44 (4H, m), 3.62 (2H, s), 3.97-4.10 (1H, m), 6.94-7.08 (1H, m), 7.54 (1H, d), 7.82 (1H, s), 7.88 (1H, s), 8.02 (1H, s), 8.16 (1H, d).

Step 11. Preparation of 5-bromo-1,15,21-trimethyl-23-oxa-2,9,11,15,20,21,26-heptaazahexacyclo[24.4.1.1\hat{ }{13,17}.0\hat{ }{2,10}.0\hat{ }{3,8}.0\hat{ }{18,22}]dotriaconta-3,5,7,9,13,17(32),18(22),19-octaene-12,16-dione (INT-A13-11). To a stirred mixture of 5-(5-{2-[3-(2-amino-6-bromo-1,3-benzodiazol-1-yl)-3-methylazepan-1-yl]ethoxy}-1-methylpyrazol-4-yl)-1-methyl-6-oxopyridine-3-carboxylic acid (140 mg, 0.23 mmol, 1 equiv) and N,N,N,N-tetramethyl-O-(7-azabenzotriazol-1-yl)uronium hexafluorophospate (133 mg, 0.35 mmol, 1.5 equiv) in dioxane (4 mL) was added N,N-diisopropylethylamine (90 mg, 0.70 mmol, 3.0 equiv) under nitrogen atmosphere. The resulting mixture was stirred at 60 °C for 2 h, cooled to room temperature, and concentrated under reduced pressure. The residue was treated with water (30 mL) and the mixture was extracted with dichloromethane (3×30 mL). The combined organic layers were washed with brine (3×30 mL), dried over anhydrous Na₂SO₄, and concentrated under reduced pressure. The residue was purified by Prep-TLC eluted with CH₂Cl₂/methanol (12/1) to give 100 mg of 5-bromo-1,15,21-trimethyl-23-oxa-2,9,11,15,20,21,26-heptaazahexacyclo[24.4.1.1\hat{ }{13,17}.0\hat{ }{2,10}.0\hat{ }{3,8}.0\hat{ }{18,22}]dotriaconta-3,5,7,9,13,17(32),18(22),19-octaene-12,16-dione (INT-A13-11) as a brown solid (73%). ¹H NMR (400 MHz, DMSO-d₆) δ 1.23-1.29 (7H, m), 1.68-1.72 (2H, m), 1.78-1.86 (5H, m), 1.87-1.91 (1H, m), 2.02-2.13 (1H, m), 2.69 (3H, s), 3.10-3.21 (3H, m), 3.23 (1H, t), 3.41 (11H, d), 3.60-3.66 (4H, m), 3.72 (3H, s), 4.30-4.47 (3H, m), 7.35-7.44 (11H, m), 7.57 (1H, d), 7.69 (1H, d), 8.10 (1H, s), 8.30 (1H, d), 8.62 (1H, d), 13.06 (1H, s).

Step 12. Preparation of 1,15,21-trimethyl-5-(morpholin-4-yl)-23-oxa-2,9,11,15,20,21,26heptaazahexacyclo[24.4.1.1\hat{ }{13,17}.0\hat{ }{2,10}.0\hat{ }{3,8}.0\hat{ }{18,22}]dotriac onta-3,5,7,9,13,17(32),18(22),19-octaene-12,16-dione (EXAMPLE A13). To a stirred mixture of 5-bromo-1,15,21-trimethyl-23-oxa-2,9,11,15,20,21,26-heptaazahexacyclo[24.4.1.1\hat{ }{13,17}.0\hat{ }{2,10}.0\hat{ }{3,8}.0\hat{ }{18,22}]dotriaconta-3,5,7,9,13,17(32),18(22),19-octaene-12,16-dione (90 mg, 0.15 mmol, 1 equiv) and BrettPhos Pd G₃ (42 mg, 0.046 mmol, 0.3 equiv) in 1,4-dioxane (3 mL) were added morpholine (54 mg, 0.62 mmol, 4 equiv) and LiHMDS (0.93 mL, 0.93 mmol, 6 equiv). The mixture was stirred for 1 h at 60 °C and then cooled to room temperature. The reaction was quenched with water (30 mL) and the mixture was extracted with ethyl acetate (3×30 mL). The combined organic layers were washed with brine (3×30 mL), dried over anhydrous Na₂SO₄, and concentrated under reduced pressure. The residue was purified by Prep-TLC eluted with CH₂Cl₂/methanol (25/1) to give 25.8 mg of 1,15,21-trimethyl-5-(morpholin-4-yl)-23-oxa-2,9,11,15,20,21,26heptaazahexacyclo[24.4.1.1\hat{ }{13,17}.0\hat{ }{2,10}.0\hat{ }{3,8}.0\hat{ }{18,22}]dotriac onta-3,5,7,9,13,17(32),18(22),19-octaene-12,16-dione (EXAMPLE A13) as a white solid (28%). LCMS: m/z (ESI), [M+H]⁺=587.40. ¹H NMR (400 MHz, DMSO-d₆) δ 1.60-1.75 (3H, m) 1.80-1.88 (4H, s), 1.96-2.06 (1H, m), 2.57-2.67 (1H, m), 2.85-2.93 (1H, m), 2.95-3.09 (3H, m), 3.06-3.15 (2H, m), 3.21-3.29 (2H, m), 3.61 (3H, s), 3.70-3.80 (8H, m), 4.32 (1H, d), 4.39 (1H, t), 4.53 (1H, d), 6.94 (1H, d), 7.04 (1H, d), 7.49 (1H, d), 8.09 (1H, s), 8.26 (1H, d), 8.59 (1H, d), 12.85 (1H, s).

### Example A14

### (11R)-26-Ethyl-5,11-dimethyl-16-(4-methylpiperazin-1-yl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione

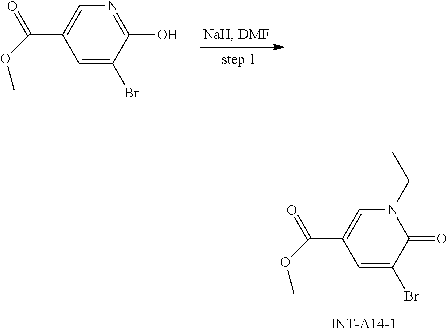

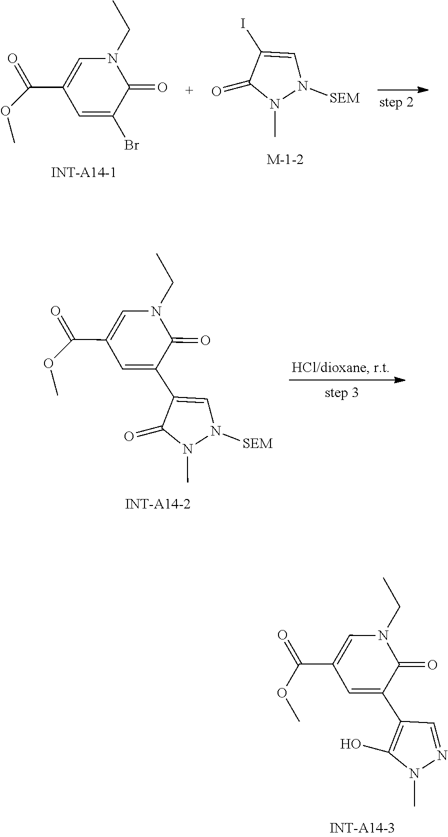 [SMILES: CCn1cc(C(=O)OC)cc(Br)c1=O ; Cn1c(=O)c(I)cn1COCC[Si](C)(C)C ; CCn1cc(C(=O)OC)cc(-c2cn(COCC[Si](C)(C)C)n(C)c2=O)c1=O ; CCn1cc(C(=O)OC)cc(-c2cnn(C)c2O)c1=O]

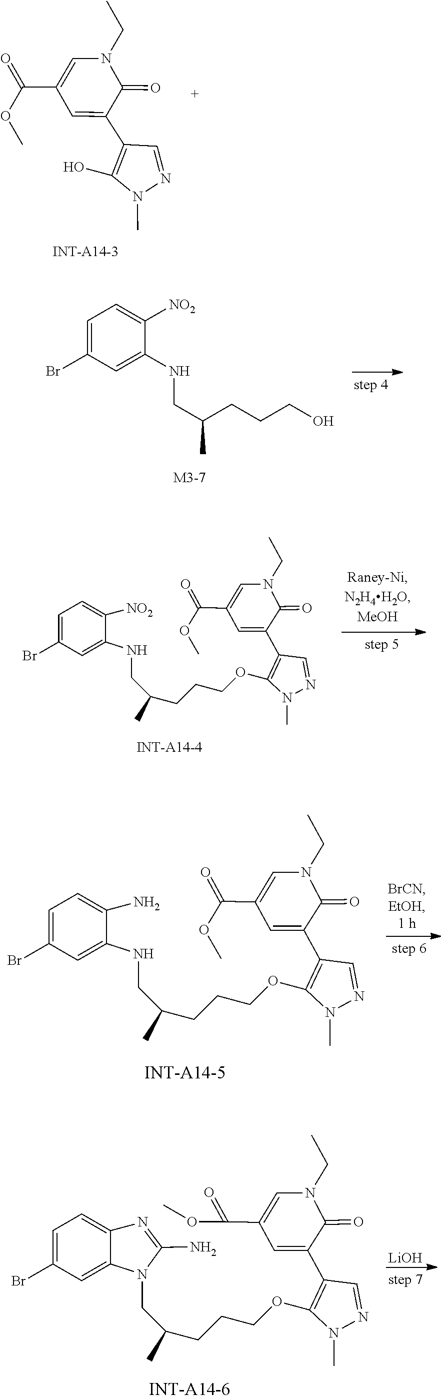

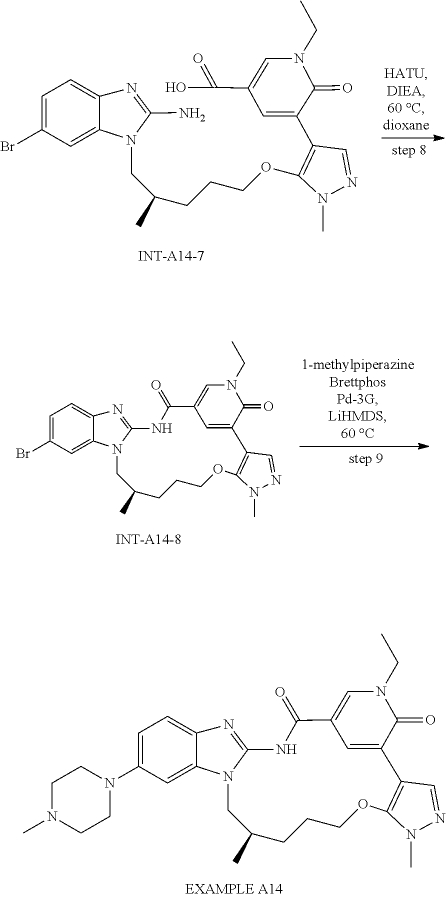 [SMILES: CCn1cc(C(=O)O)cc(-c2cnn(C)c2OCCC[C@@H](C)Cn2c(N)nc3ccc(Br)cc32)c1=O ; CCn1cc2cc(c1=O)-c1cnn(C)c1OCCC[C@@H](C)Cn1c(nc3ccc(Br)cc31)NC2=O ; CCn1cc2cc(c1=O)-c1cnn(C)c1OCCC[C@@H](C)Cn1c(nc3ccc(N4CCN(C)CC4)cc31)NC2=O]

Step 1. Preparation of methyl 5-bromo-1-ethyl-6-oxo-1,6-dihydropyridine-3-carboxylate (INT-A14-1). To a stirred mixture of methyl 5-bromo-6-hydroxypyridine-3-carboxylate (10 g, 43.09 mmol, 1 equiv) in N,N-dimethylformamide (200 mL) was added NaH (3.45 g, 86.19 mmol, 2 equiv, 60%) at 0 °C. The reaction mixture was stirred at 60 °C for 30 mins prior to the addition of ethyl iodide (10.08 g, 64.64 mmol, 1.5 equiv). The mixture was stirred for additional 2 h at room temperature, treated with water (1 L), and extracted with ethyl acetate (3×1 L). The combined organic layers were washed with brine (3×1 L), dried over anhydrous Na₂SO₄, and concentrated under reduced pressure. The residue was purified by silica gel chromatography eluted with petroleum ether/ethyl acetate (3/1) to give 5 g of methyl 5-bromo-1-ethyl-6-oxopyridine-3-carboxylate (INT-A14-1) as a yellow solid (37%). LCMS: m/z (ESI), [M+H]⁺=261.90. ¹H NMR (400 MHz, DMSO-d₆) δ 1.25 (3H, t), 3.81 (3H, s), 4.08 (2H, q), 8.17 (1H, d), 8.61 (1H, d)

Step 2. Preparation of methyl-1-ethyl-5-(2-methyl-3-oxo-1-((2-(trimethylsilyl)ethoxy)methyl)-2,3-dihydro-1H-pyrazol-4-yl)-6-oxo-1,6-dihydropyridine-3-carboxylate (INT-A14-2). A mixture of methyl 5-bromo-1-ethyl-6-oxopyridine-3-carboxylate (INT-A14-1, 2.0 g, 7.69 mmol, 1 equiv), 4,4,5,5-tetramethyl-2-(tetramethyl-1,3,2-dioxaborolan-2-yl)-1,3,2-dioxaborolane (2.93 g, 11.53 mmol, 1.5 equiv), Pd(dppf)Cl₂ (0.56 g, 0.77 mmol, 0.1 equiv), and KOAc (2.26 g, 23.07 mmol, 3.0 equiv) in dioxane (25 mL) was stirred for 1 h at 60 °C under nitrogen atmosphere. The mixture was cooled to room temperature, followed by the addition of 4-iodo-2-methyl-1-{[2-(trimethylsilyl)ethoxy]methyl}pyrazol-3-one (2.72 g, 7.69 mmol, 1.0 equiv), Pd(dppf)Cl₂ (1.13 g, 1.54 mmol, 0.2 equiv), K₂CO₃ (3.19 g, 23.07 mmol, 3.0 equiv), and H₂O (8 mL). The reaction mixture was stirred at 80 °C for 2 h under nitrogen atmosphere. After being cooled to room temperature, the reaction was quenched with water (300 mL) and the mixture was extracted with ethyl acetate (3×300 mL). The combined organic layers were washed with brine (3×300 mL), dried over anhydrous Na₂SO₄, and concentrated under reduced pressure. The residue was purified by silica gel chromatography eluted with dichloromethane/methanol (12/1) to give 1 g of methyl 1-ethyl-5-(2-methyl-3-oxo-1-{[2-(trimethylsilyl)ethoxy]methyl}pyrazol-4-yl)-6-oxopyridine-3-carboxylate (INT-A14-2) as a brown solid (31%). LCMS: m/z (ESI), [M+H]⁺=408.15. ¹H NMR (400 MHz, DMSO-d₆) δ 0.05 (9H, s), 0.78-0.86 (2H, m), 1.27 (3H, t), 3.37 (3H, s), 3.43-3.50 (2H, m), 3.82 (3H, s), 4.08-4.12 (2H, m), 5.32 (2H, s), 8.41 (1H, d), 8.87 (1H, s), 9.20 (1H, d).

Step 3. Preparation of methyl 1-ethyl-5-(5-hydroxy-1-methyl-1H-pyrazol-4-yl)-6-oxo-1,6-dihydropyridine-3-carboxylate (INT-A14-3). A mixture of methyl 1-ethyl-5-(2-methyl-3-oxo-1-{[2-(trimethylsilyl)ethoxy]methyl}pyrazol-4-yl)-6-oxopyridine-3-carboxylate (1.0 g, 2.45 mmol, 1 equiv) and HCl (10 mL, 4 M in 1,4-dioxane) was stirred overnight at room temperature. The mixture was concentrated under vacuum. The residue was treated with CH₂Cl₂ (20 mL) and saturated NaHCO₃ (aq.) (50 mL). The resulting mixture was extracted with dichloromethane (3×100 mL). The combined organic layers were washed with brine (3×50 mL), dried over anhydrous Na₂SO₄, and concentrated under reduced pressure. The residue was purified by reverse flash chromatography eluted with 0% to 100% acetonitrile in water to give 520 mg of methyl 1-ethyl-5-(5-hydroxy-1-methylpyrazol-4-yl)-6-oxopyridine-3-carboxylate (INT-A14-3) as a yellow solid (76%). LCMS: m/z (ESI), [M+H]⁺=278.05. ¹H NMR (400 MHz, DMSO-d₆) δ 1.29 (3H, t), 3.33 (3H, s), 3.84 (3H, s), 4.13-4.17 (2H, m), 7.96 (1H, s), 8.45 (2H, d), 12.72-13.41 (1H, br s).

Step 4. Preparation of methyl-(R)-5-(5-((5-((5-bromo-2-nitrophenyl)amino)-4-methylpentyl)oxy)-1-methyl-1H-pyrazol-4-yl)-1-ethyl-6-oxo-1,6-dihydropyridine-3-carboxylate (INT-A14-4). To a stirred mixture of methyl 1-ethyl-5-(5-hydroxy-1-methylpyrazol-4-yl)-6-oxopyridine-3-carboxylate (500 mg, 1.80 mmol, 1 equiv) and triphenylphosphine (1.41 g, 5.41 mmol, 3 equiv) in tetrahydrofuran (8 mL) were added diisopropyl azodicarboxylate (1.09 g, 5.41 mmol, 3 equiv) and (4R)-5-[(5-bromo-2-nitrophenyl)amino]-4-methylpentan-1-ol (M3-7, 1.14 g, 3.60 mmol, 2.0 equiv) at 0 °C under nitrogen atmosphere. The resulting mixture was stirred for 2 h at 0 °C. The reaction was quenched with water (100 mL) and the mixture was extracted with dichloromethane (3×100 mL). The combined organic layers were washed with brine (3×100 mL), dried over anhydrous Na₂SO₄, and concentrated under reduced pressure. The residue was purified by reverse flash chromatography eluted with 0% to 100% acetonitrile in water to give 370 mg of methyl-(R)-5-(5-((5-((5-bromo-2-nitrophenyl)amino)-4-methylpentyl)oxy)-1-methyl-1H-pyrazol-4-yl)-1-ethyl-6-oxo-1,6-dihydropyridine-3-carboxylate (INT-A14-4) as a yellow solid (35%). LCMS: m/z (EST), [M+H]⁺=577.90. ¹H NMR (DMSO-d₆, 400 MHz) δ 0.96 (3H, d), 1.27 (3H, t), 1.29-1.45 (1H, m), 1.58-1.64 (1H, m), 1.69-1.96 (3H, m), 3.16-3.27 (1H, m), 3.27-3.38 (1H, m), 3.68 (3H, s), 3.78 (3H, s), 3.96 (2H, t), 4.08 (2H, q), 6.80-6.86 (1H, m), 7.26 (1H, d), 7.95-8.02 (2H, m), 8.13 (1H, d), 8.21 (1H, t), 8.43 (1H, d)

Step 5. Preparation of methyl (R)-5-(5-((5-((2-amino-5-bromophenyl)amino)-4-methylpentyl)oxy)-1-methyl-1H-pyrazol-4-yl)-1-ethyl-6-oxo-1,6-dihydropyridine-3-carboxylate (INT-A14-5). To a stirred mixture of Raney-Ni (53 mg, 0.62 mmol, 1 equiv) and methyl-(R)-5-(5-((5-((5-bromo-2-nitrophenyl)amino)-4-methylpentyl)oxy)-1-methyl-1H-pyrazol-4-yl)-1-ethyl-6-oxo-1,6-dihydropyridine-3-carboxylate (INT-A14-4, 360 mg, 0.62 mmol, 1 equiv) in methanol (5 mL) was added NH₂NH₂H₂O (62 mg, 1.25 mmol, 2 equiv) at 0 °C under nitrogen atmosphere. After being stirred for 1 h at 60 °C under, the resulting mixture was filtered and the organic solution was concentrated under reduced pressure to give 350 mg of methyl (R)-5-(5-((5-((2-amino-5-bromophenyl)amino)-4-methylpentyl)oxy)-1-methyl-1H-pyrazol-4-yl)-1-ethyl-6-oxo-1,6-dihydropyridine-3-carboxylate (INT-A14-5) as a brown solid. This material was directly used for the next reaction without purification.

Step 6. Preparation of methyl-(R)-5-(5-((5-(2-amino-6-bromo-1H-benzo[d]imidazol-1-yl)-4-methylpentyl)oxy)-1-methyl-1H-pyrazol-4-yl)-1-ethyl-6-oxo-1,6-dihydropyridine-3-carboxylate (INT-A14-6). To the solution of freshly prepared (R)-5-(5-((5-((2-amino-5-bromophenyl)amino)-4-methylpentyl)oxy)-1-methyl-1H-pyrazol-4-yl)-1-ethyl-6-oxo-1,6-dihydropyridine-3-carboxylate (INT-A14-5, 350 mg) in alcohol (5 mL) was added BrCN (83 mg, 0.79 mmol, 1.5 equiv) at room temperature under nitrogen atmosphere. After being stirred for 1 h at 60 °C under nitrogen atmosphere, the mixture was concentrated under vacuum. The residue was purified by prep-TLC eluted with petroleum ether/ethyl acetate (5/1) to give 260 mg of methyl-(R)-5-(5-((5-(2-amino-6-bromo-1H-benzo[d]imidazol-1-yl)-4-methylpentyl)oxy)-1-methyl-1H-pyrazol-4-yl)-1-ethyl-6-oxo-1,6-dihydropyridine-3-carboxylate (INT-A14-6) as a brown solid (69%). LCMS: m/z (ESI), [M+H]⁺=573.25. ¹1H NMR (DMSO-d₆, 400 MHz) δ 0.83 (3H, d), 1.26 (3H, t), 1.28-1.40 (1H, m), 1.49-1.53 (1H, m), 1.67-1.71 (1H, m), 1.78-1.94 (1H, m), 1.96-2.02 (1H, m), 3.61 (3H, s), 3.79 (3H, s), 3.80-3.97 (4H, m), 4.08 (2H, q), 6.56 (2H, s), 7.04 (2H, d), 7.36 (1H, d), 7.99 (1H, s), 8.13 (1H, d), 8.45 (1H, d).

Step 7. Preparation of 5-(5-{[(4R)-4-[(2-amino-6-bromo-1,3-benzodiazol-1-yl)methyl]pentyl]oxy}-1-methylpyrazol-4-yl)-1-ethyl-6-oxopyridine-3-carboxylic acid (INT-A14-7). To a stirred mixture of methyl 5-(5-{[(4R)-4-[(2-amino-6-bromo-1,3-benzodiazol-1-yl)methyl]pentyl]oxy}-1-methylpyrazol-4-yl)-1-ethyl-6-oxopyridine-3-carboxylate (260 mg, 0.45 mmol, 1 equiv) in THF (2 mL) was added LiOH·H₂O (38 mg, 0.91 mmol, 2.0 equiv) in H₂O (0.5 mL) at room temperature. After being stirred for 30 min at 60 °C under nitrogen atmosphere. The mixture was cooled to room temperature and concentrated under vacuum. The residue was purified by reverse flash chromatography eluted with 0% to 20% acetonitrile in water to give 160 mg of 5-(5-{[(4R)-4-[(2-amino-6-bromo-1,3-benzodiazol-1-yl)methyl]pentyl]oxy}-1-methylpyrazol-4-yl)-1-ethyl-6-oxopyridine-3-carboxylic acid (INT-A14-7) as a white solid (63%). LCMS: m/z (ESI), [M+H]⁺=559.15.

Step 8. Preparation of (11R)-16-bromo-26-ethyl-5,11-dimethyl-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (INT-A14-8) To a stirred mixture of 5-(5-{[(4R)-4-[(2-amino-6-bromo-1,3-benzodiazol-1-yl)methyl]pentyl]oxy}-1-methylpyrazol-4-yl)-1-ethyl-6-oxopyridine-3-carboxylic acid (160 mg, 0.28 mmol, 1 equiv) and N,N,N,N-tetramethyl-O-(7-azabenzotriazol-1-yl)uronium hexafluorophospate (163 mg, 0.43 mmol, 1.5 equiv) in dioxane (2 mL) was added N,N-diisopropylethylamine (111 mg, 0.86 mmol, 3.0 equiv) at room temperature under nitrogen atmosphere. The reaction mixture was stirred at room temperature for 1 h. The reaction was quenched with water (50 mL) and the mixture was extracted with ethyl acetate (3×50 mL). The combined organic layers were washed with brine (3×50 mL), dried over anhydrous Na₂SO₄, and concentrated under reduced pressure. The residue was purified by prep-TLC eluted with dichloromethane/methanol (12/1) to give 110 mg of (11R)-16-bromo-26-ethyl-5,11-dimethyl-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (INT-A14-8) as a brown solid (71%). LCMS: m/z (EST), [M+H]⁺=541.05.

Step 9. Preparation of (11R)-26-ethyl-5,11-dimethyl-16-(4-methylpiperazin-1-yl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (EXAMPLE A14). To a stirred mixture of (11R)-16-bromo-26-ethyl-5,11-dimethyl-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (120 mg, 0.22 mmol, 1 equiv), 1-methylpiperazine (89 mg, 0.89 mmol, 4 equiv), and BrettPhos Pd G₃ (60 mg, 0.07 mmol, 0.3 equiv) in dioxane (5 mL) was added LiHMDS (223 mg, 1.33 mmol, 6 equiv) dropwise under nitrogen atmosphere. The reaction mixture was stirred at 60 °C for 30 mins. After being cooled to room temperature, the reaction was quenched with water (100 mL) and the mixture was extracted with ethyl acetate (3×100 mL). The combined organic layers were washed with brine (3×100 mL), dried over anhydrous Na₂SO₄, and concentrated under reduced pressure. The residue was purified by prep-TLC eluted with dichloromethane/methanol (15/1) to give 50 mg of (11R)-26-ethyl-5,11-dimethyl-16-(4-methylpiperazin-1-yl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (EXAMPLE A14) as a white solid (39%). LCMS: m/z (EST), [M+H]⁺=559.35. ¹H NMR (400 MHz, DMSO-d₆) δ 0.80-0.86 (4H, m), 1.13-1.34 (4H, m), 1.40-1.46 (2H, m), 1.91 (2H, d), 2.26-2.34 (5H, m), 2.80 (1H, s), 3.16 (4H, t), 3.72 (3H, s), 3.94 (2H, m), 4.05-4.18 (3H, m), 4.35 (1H, t), 6.82-6.88 (1H, m), 7.13 (1H, d), 7.36 (1H, d), 8.26 (1H, d), 8.35 (1H, s), 8.80 (1H, d), 12.35 (1H, s).

### Example A15

### (11R)-26-cyclopropyl-5,11-dimethyl-16-(4-methylpiperazin-1-yl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione

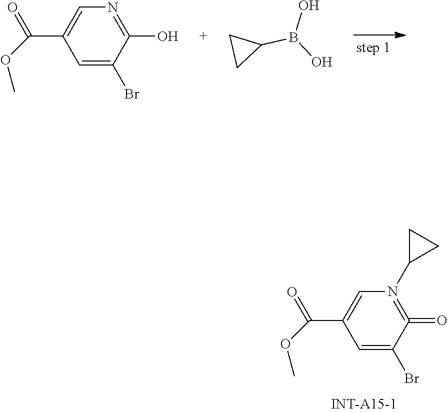

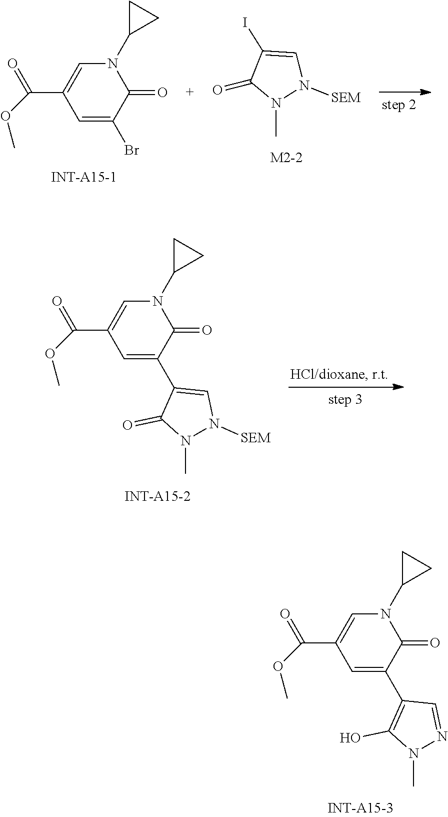 [SMILES: COC(=O)c1cc(Br)c(=O)n(C2CC2)c1 ; Cn1c(=O)c(I)cn1COCC[Si](C)(C)C ; COC(=O)c1cc(-c2cn(COCC[Si](C)(C)C)n(C)c2=O)c(=O)n(C2CC2)c1 ; COC(=O)c1cc(-c2cnn(C)c2O)c(=O)n(C2CC2)c1]

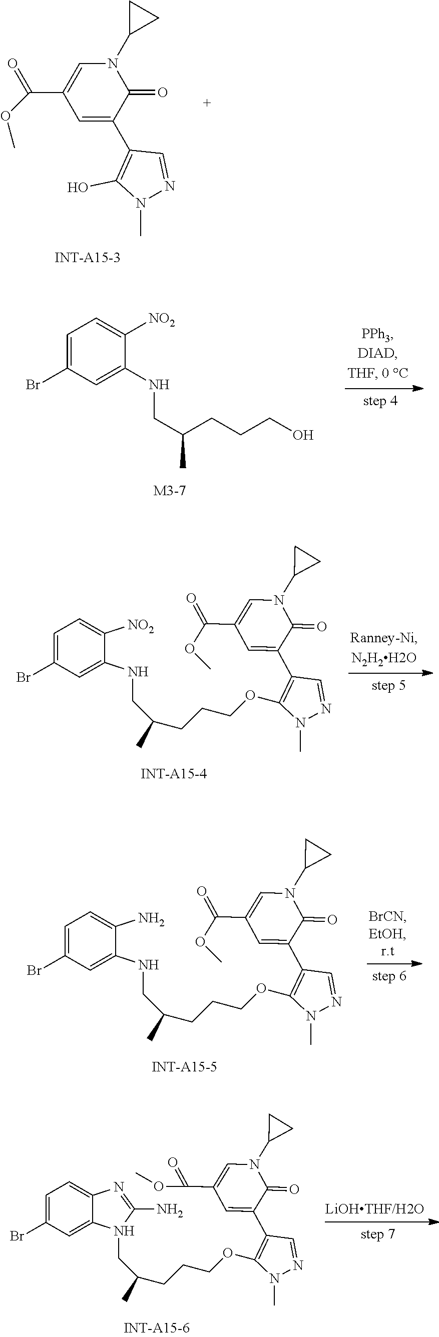 [SMILES: COC(=O)c1cc(-c2cnn(C)c2O)c(=O)n(C2CC2)c1 ; C[C@H](CCCO)CNc1cc(Br)ccc1[N+](=O)[O-] ; COC(=O)c1cc(-c2cnn(C)c2OCCC[C@@H](C)CNc2cc(Br)ccc2[N+](=O)[O-])c(=O)n(C2CC2)c1 ; COC(=O)c1cc(-c2cnn(C)c2OCCC[C@@H](C)CNc2cc(Br)ccc2N)c(=O)n(C2CC2)c1 ; COC(=O)c1cc(-c2cnn(C)c2OCCC[C@@H](C)Cn2c(N)nc3ccc(Br)cc32)c(=O)n(C2CC2)c1]

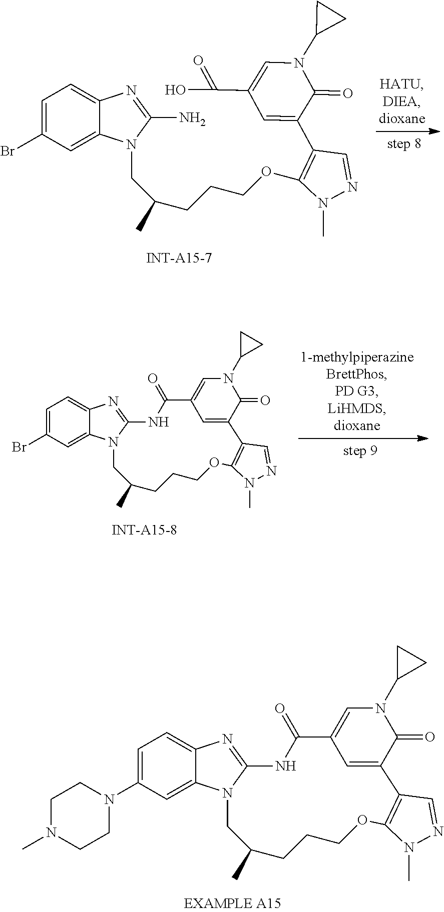 [SMILES: C[C@H](CCCOc1c(-c2cc(C(=O)O)cn(C3CC3)c2=O)cnn1C)Cn1c(N)nc2ccc(Br)cc21 ; C[C@@H]1CCCOc2c(cnn2C)-c2cc(cn(C3CC3)c2=O)C(=O)Nc2nc3ccc(Br)cc3n2C1 ; C[C@@H]1CCCOc2c(cnn2C)-c2cc(cn(C3CC3)c2=O)C(=O)Nc2nc3ccc(N4CCN(C)CC4)cc3n2C1]

Step 1. Preparation of methyl 5-bromo-1-cyclopropyl-6-oxopyridine-3-carboxylate (INT-A15-1). A mixture of methyl 5-bromo-6-hydroxypyridine-3-carboxylate (5 g, 21.55 mmol, 1 equiv), cyclopropylboronic acid (4.07 g, 47.41 mmol, 2.2 equiv), Cu(OAc)₂ (4.31 g, 23.70 mmol, 1.1 equiv) and trimethylamine (4.36 g, 43.10 mmol, 2 equiv) in acetonitrile (100 mL) and ethanol (5 mL) was stirred for 12 h at 80 °C under nitrogen atmosphere. The mixture was cooled to room temperature and filtered. The filter cake was washed with ethyl acetate (3×400 mL) and the resulting solution was concentrated under reduced pressure. The residue was purified on a silica gel column eluted with petroleum ether/ethyl acetate (1/1) to give 350 mg of methyl 5-bromo-1-cyclopropyl-6-oxopyridine-3-carboxylate (INT-A15-1) as a yellow solid (30%). LCMS: m/z (ESI), [M+H]⁺=271.90. ¹H NMR (DMSO-d₆, 400 MHz) δ 0.90-0.97 (2H, m), 1.01-1.09 (2H, m), 3.38-3.42 (1H, m), 3.80 (3H, s), 8.17 (1H, d), 8.22 (1H, d).

Step 2. Preparation of methyl 1-cyclopropyl-5-(2-methyl-3-oxo-1-{[2-(trimethylsilyl)ethoxy]methyl}pyrazol-4-yl)-6-oxopyridine-3-carboxylate (INT-A15-2). A mixture of methyl 5-bromo-1-cyclopropyl-6-oxopyridine-3-carboxylate (400 mg, 1.47 mmol, 1 equiv) and 4,4,5,5-tetramethyl-2-(tetramethyl-1,3,2-dioxaborolan-2-yl)-1,3,2-dioxaborolane (560 mg, 2.20 mmol, 1.5 equiv), Pd(dppf)Cl₂CH₂Cl₂ (239 mg, 0.29 mmol, 0.2 equiv), and KOAc (432 mg, 4.41 mmol, 3 equiv) in dioxane (8 mL) was stirred for 1 h at 80 °C under nitrogen atmosphere and then cooled to room temperature. To the above mixture were added 4-iodo-2-methyl-1-{[2-(trimethylsilyl)ethoxy]methyl}pyrazol-3-one (520.79 mg, 1.470 mmol, 1 equiv), K₂CO₃ (406 mg, 2.94 mmol, 2 equiv), and water (2 mL). The resulting mixture was stirred for additional 2 h at 80 °C. After being cooled to room temperature, the reaction was quenched with water (100 mL) and the mixture was extracted with dichloromethane (3×100 mL). The combined organic layers were washed with brine (3×100 mL), dried over anhydrous Na₂SO₄, and concentrated under reduced pressure. The residue was purified by prep-TLC eluted with dichloromethane/methanol (1/1) to give 210 mg of methyl 1-cyclopropyl-5-(2-methyl-3-oxo-1-{[2-(trimethylsilyl)ethoxy]methyl}pyrazol-4-yl)-6-oxopyridine-3-carboxylate (INT-A15-2) as a yellow solid (34%). LCMS: m/z (ESI), [M+H]⁺=420.15. ¹H NMR (DMSO-d₆, 400 MHz) δ 0.06 (9H, s), 0.83 (2H, t), 0.89-0.95 (2H, m), 1.04-1.10 (2H, m), 3.24-3.26 (1H, m), 3.37 (3H, s), 3.45-3.50 (2H, m), 3.81 (3H, d), 5.32 (2H, s), 8.06 (1H, d), 8.89 (1H, s), 9.18 (1H, d).

Step 3. Preparation of methyl 1-cyclopropyl-5-(5-hydroxy-1-methylpyrazol-4-yl)-6-oxopyridine-3-carboxylate (INT-A15-3). A solution of methyl 1-cyclopropyl-5-(2-methyl-3-oxo-1-{[2-(trimethylsilyl)ethoxy]methyl}pyrazol-4-yl)-6-oxopyridine-3-carboxylate (210 mg, 0.50 mmol, 1 equiv) and HCl (5 mL, 4M in 1,4-dioxane) was stirred for 6 h at room temperature. The resulting mixture was concentrated under reduced pressure. The residue was treated with CH₂Cl₂ (20 mL) and saturated NaHCO₃ (aq.) (50 mL). The mixture was extracted with dichloromethane (3×100 mL). The combined organic layers were washed with brine (3×50 mL), dried over anhydrous Na₂SO₄, and concentrated under reduced pressure to give 190 mg of methyl 1-cyclopropyl-5-(5-hydroxy-1-methylpyrazol-4-yl)-6-oxopyridine-3-carboxylate (INT-A15-3) which was used in the next step directly without further purification. LCMS: m/z (EST), [M+H]⁺=290.00. ¹H NMR (DMSO-d₆, 400 MHz) δ 0.95-1.00 (2H, m), 1.08-1.12 (2H, m), 3.45-3.49 (1H, m), 3.51 (3H, s), 3.83 (3H, s), 7.46-7.69 (1H, m), 8.03 (1H, s), 8.08 (1H, d), 8.38 (1H, d).

Step 4. Preparation of methyl 5-(5-{[(4R)-5-[(5-bromo-2-nitrophenyl)amino]-4-methylpentyl]oxy}-1-methylpyrazol-4-yl)-1-cyclopropyl-6-oxopyridine-3-carboxylate (INT-A 15-4). To a solution of methyl 1-cyclopropyl-5-(5-hydroxy-1-methylpyrazol-4-yl)-6-oxopyridine-3-carboxylate (crude, 190 mg, 0.66 mmol, 1 equiv) and triphenyl phosphine (516 mg, 1.97 mmol, 3 equiv) in tetrahydrofuran (10 mL) were added diisopropyl azodicarboxylate (398 mg, 1.97 mmol, 3 equiv) and (4R)-5-[(5-bromo-2-nitrophenyl)amino]-4-methylpentan-1-ol (208 mg, 0.66 mmol, 1 equiv) at 0 °C under nitrogen atmosphere. The resulting mixture was stirred for 3 hours at 0 °C. The reaction was quenched with water (100 mL) and the mixture was extracted with ethyl acetate (3×100 mL). The combined organic layers were washed with brine (3×100 mL), dried over anhydrous Na₂SO₄, and concentrated under reduced pressure. The residue was purified by Prep-TLC eluted with ethyl acetate to give 135 mg of methyl 5-(5-{[(4R)-5-[(5-bromo-2-nitrophenyl)amino]-4-methylpentyl]oxy}-1-methylpyrazol-4-yl)-1-cyclopropyl-6-oxopyridine-3-carboxylate (INT-A15-4) as a yellow solid (33%). LCMS: m/z (ESI), [M+H]⁺=589.90. ¹H NMR (DMSO-d₆, 400 MHz) δ 0.85-1.00 (5H, m), 1.02-1.12 (2H, m), 1.15-1.30 (2H, m), 1.30-1.52 (1H, m), 1.53-1.96 (3H, m), 3.19-3.23 (1H, m), 3.38-3.42 (1H, m), 3.68 (3H, s), 3.77 (3H, s), 3.95 (2H, t), 6.80-6.86 (1H, m), 7.26 (1H, d), 7.97-8.01 (2H, m), 8.06 (1H, d), 8.11 (1H, d), 8.20 (1H, t)

Step 5. Preparation of methyl 5-(5-{[(4R)-5-[(2-amino-5-bromophenyl)amino]-4-methylpentyl]oxy}-1-methylpyrazol-4-yl)-1-cyclopropyl-6-oxopyridine-3-carboxylate (INT-A15-5). To a mixture of Raney-Ni (60 mg, 0.70 mmol, 3 equiv) and methyl 5-(5-{[(4R)-5-[(5-bromo-2-nitrophenyl)amino]-4-methylpentyl]oxy}-1-methylpyrazol-4-yl)-1-cyclopropyl-6-oxopyridine-3-carboxylate (135 mg, 0.2 mmol, 1 equiv) in methanol (3 mL) was added hydrazine hydrate (23 mg, 0.46 mmol, 2 equiv) at 0 °C under nitrogen atmosphere. The resulting mixture was stirred for 2 h at room temperature. The resulting mixture was filtered. The filter cake was washed with methanol (3×50 mL). The resulting solution was concentrated under reduced pressure. The residue was purified by Prep-TLC eluted with dichloromethane/methanol (16/1) to give 85 mg of methyl 5-(5-{[(4R)-5-[(2-amino-5-bromophenyl)amino]-4-methylpentyl]oxy}-1-methylpyrazol-4-yl)-1-cyclopropyl-6-oxopyridine-3-carboxylate (INT-A15-5) as a brown solid (65%). LCMS: m/z (ESI), [M+H]=558.05. ¹H NMR (DMSO-d₆, 400 MHz) δ 0.80-1.12 (7H, m), 1.20-1.40 (2H, m), 1.52-1.95 (4H, m), 2.70-3.02 (2H, m), 3.68 (3H, s), 3.79 (3H, s), 3.96 (2H, t), 4.59-4.75 (3H, m), 6.41-6.54 (3H, m), 8.01 (1H, s), 8.09 (1H, d), 8.14 (1H, d)

Step 6. Preparation of methyl 5-(5-{[(4R)-4-[(2-amino-6-bromo-1,3-benzodiazol-1-yl)methyl]pentyl]oxy}-1-methylpyrazol-4-yl)-1-cyclopropyl-6-oxopyridine-3-carboxylate (INT-A15-6). A mixture of methyl 5-(5-{[(4R)-5-[(2-amino-5-bromophenyl)amino]-4-methylpentyl]oxy}-1-methylpyrazol-4-yl)-1-cyclopropyl-6-oxopyridine-3-carboxylate (85 mg, 0.15 mmol, 1 equiv) and cyanogen bromide (24 mg, 0.23 mmol, 1.5 equiv) in ethanol (18 mL) was stirred for 3 h at room temperature. The resulting mixture was concentrated under reduced pressure and the residue was purified by Prep-TLC eluted with dichloromethane/methanol (12/1) to give 60 mg of methyl 5-(5-{[(4R)-4-[(2-amino-6-bromo-1,3-benzodiazol-1-yl)methyl]pentyl]oxy}-1-methylpyrazol-4-yl)-1-cyclopropyl-6-oxopyridine-3-carboxylate (INT-A15-6) as a brown solid (65%). LCMS: m/z (ESI), [M+H]⁺=585.05.

Step 7. Preparation of 5-(5-{[(4R)-4-[(2-amino-6-bromo-1,3-benzodiazol-1-yl)methyl]pentyl]oxy}-1-methylpyrazol-4-yl)-1-cyclopropyl-6-oxopyridine-3-carboxylic acid (INT-A15-7). To a mixture of methyl 5-(5-{[(4R)-4-[(2-amino-6-bromo-1,3-benzodiazol-1-yl)methyl]pentyl]oxy}-1-methylpyrazol-4-yl)-1-cyclopropyl-6-oxopyridine-3-carboxylate (70 mg, 0.12 mmol, 1 equiv) in tetrahydrofuran (4 mL) was added LiOH (11 mg, 0.48 mmol, 4 equiv) in water (1 mL). After being stirred for 2 h at room temperature, the mixture was concentrated under reduced pressure. The residue was purified by reverse flash chromatography eluted with 10% to 50% acetonitrile in water to give 50 mg of 5-(5-{[(4R)-4-[(2-amino-6-bromo-1,3-benzodiazol-1-yl)methyl]pentyl]oxy}-1-methylpyrazol-4-yl)-1-cyclopropyl-6-oxopyridine-3-carboxylic acid (INT-A15-7) as an off-white solid (73%). LCMS: m/z (EST), [M+H]⁺=569.25.

Step 8. Preparation of (11R)-16-bromo-26-cyclopropyl-5,11-dimethyl-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (INT-A15-8). A mixture of 5-(5-{[(4R)-4-[(2-amino-6-bromo-1,3-benzodiazol-1-yl)methyl]pentyl]oxy}-1-methylpyrazol-4-yl)-1-cyclopropyl-6-oxopyridine-3-carboxylic acid (60 mg, 0.10 mmol, 1 equiv), N,N-diisopropylethylamine (27 mg, 0.21 mmol, 2 equiv) and N,N,N,N-tetramethyl-O-(7-azabenzotriazol-1-yl)uronium hexafluorophospate (80 mg, 0.21 mmol, 2 equiv) in 1,4-dioxane (1 mL, 11.80 mmol, 112 equiv) was stirred for 2 hours at room temperature under nitrogen atmosphere. The reaction was quenched with water (50 mL) and the mixture was extracted with ethyl acetate (3×50 mL). The combined organic layers were washed with brine (3×50 mL), dried over anhydrous Na₂SO₄, and concentrated under reduced pressure. The residue was purified by Prep-TLC eluted with dichloromethane/methanol (25/1) to give 27 mg of (11R)-16-bromo-26-cyclopropyl-5,11-dimethyl-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (INT-A15-8) as a yellow solid (46%). LCMS: m/z (ESI), [M+H]⁺=553.10.

Step 9. Preparation of (11R)-26-cyclopropyl-5,11-dimethyl-16-(4-methylpiperazin-1-yl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (EXAMPLE A15). To a stirred mixture of (11R)-16-bromo-26-cyclopropyl-5,11-dimethyl-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (20 mg, 0.04 mmol, 1 equiv), 1-methylpiperazine (14.53 mg, 0.144 mmol, 4 equiv), and BrettPhos Pd G₃ (9.86 mg, 0.01 mmol, 0.3 equiv) in dioxane (1 mL) was added LiHMDS (0.14 mL, 0.144 mmol, 4 equiv) dropwise under nitrogen atmosphere. The reaction mixture was stirred at 60 °C for 30 mins. After being cooled to room temperature, the reaction was quenched with water (50 mL) and the mixture was extracted with ethyl acetate (3×50 mL). The combined organic layers were washed with brine (3×50 mL), dried over anhydrous Na₂SO₄, and concentrated under reduced pressure. The residue was purified by reverse flash chromatography eluted with 10% to 50% acetonitrile in water to give 8.6 mg of (11R)-26-cyclopropyl-5,11-dimethyl-16-(4-methylpiperazin-1-yl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (EXAMPLE A15) as a white solid (41%). LCMS: m/z (ESI), [M+H]⁺=571.35. ¹H NMR (DMSO-d₆, 400 MHz) δ 0.80 (3H, d), 0.87-0.89 (2H, m), 1.09-1.13 (2H, m), 1.42-1.48 (1H, m), 1.90-1.92 (2H, m), 2.18-2.28 (4H, m), 2.50 (4H, t), 2.78-2.82 (1H, m), 3.16 (4H, t), 3.42-3.46 (1H, m), 3.72 (3H, s), 3.87-3.94 (2H, m), 4.09 (1H, d), 4.33 (1H, t), 6.84-6.88 (1H, m), 7.14 (1H, d), 7.31-7.37 (1H, m), 8.04 (1H, d), 8.35 (1H, s), 8.77 (1H, d), 12.38 (1H, s).

### Example A16A and Example A16B

### 11-Ethyl-5,26-dimethyl-16-(4-methylpiperazin-1-yl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione, Isomer 1 (Example A16A) and Isomer 2 (Example A16B)

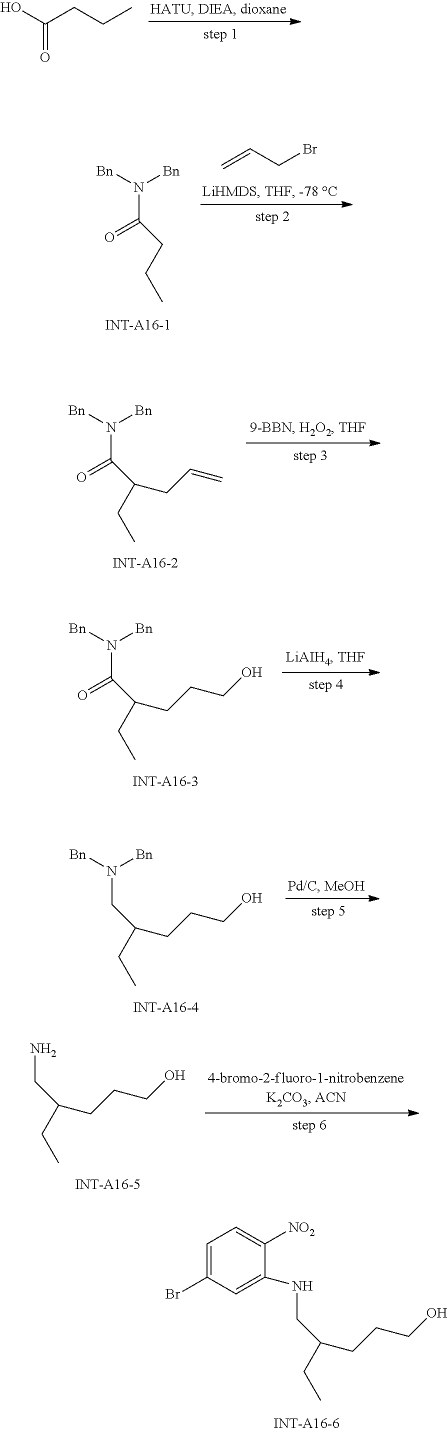

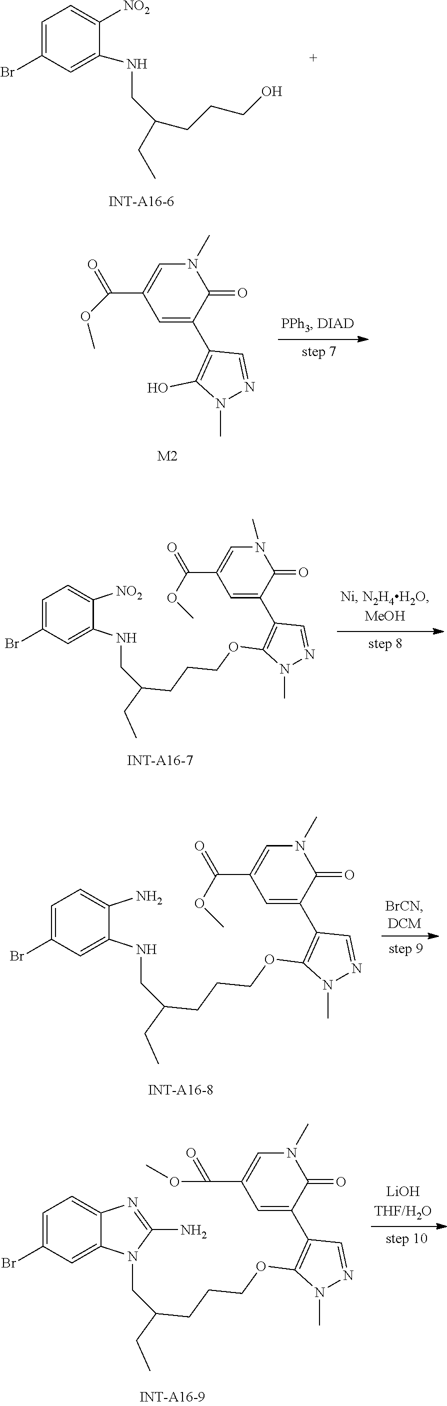 [SMILES: CCC(CCCO)CNc1cc(Br)ccc1[N+](=O)[O-] ; COC(=O)c1cc(-c2cnn(C)c2O)c(=O)n(C)c1 ; "M2" ; CCC(CCCOc1c(-c2cc(C(=O)OC)cn(C)c2=O)cnn1C)CNc1cc(Br)ccc1[N+](=O)[O-] ; CCC(CCCOc1c(-c2cc(C(=O)OC)cn(C)c2=O)cnn1C)CNc1cc(Br)ccc1N ; CCC(CCCOc1c(-c2cc(C(=O)OC)cn(C)c2=O)cnn1C)Cn1c(N)nc2ccc(Br)cc21]

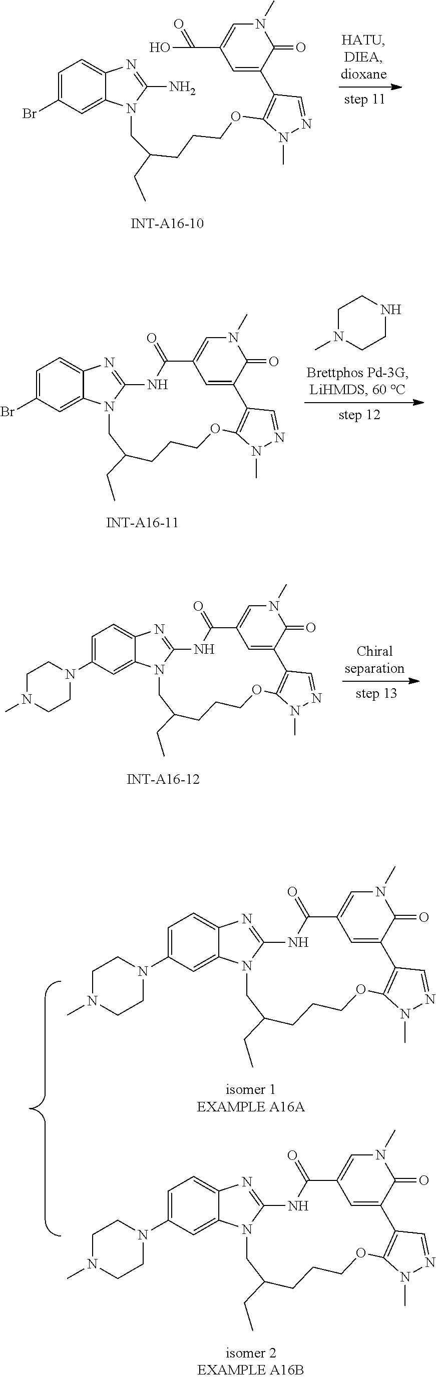

Step 1. Preparation of N,N-dibenzylbutanamide (INT-A16-1). To a stirred mixture of butanoic acid (10 g, 113.50 mmol, 1 equiv) and dibenzyl amine (22.39 g, 113.50 mmol, 1 equiv) in 1,4-dioxane (100 mL) were added N,N,N,N-tetramethyl-O-(7-azabenzotriazol-1-yl)uronium hexafluorophospate (64.73 g, 170.25 mmol, 1.5 equiv) and N,N-diisopropylethylamine (44.01 g, 340.50 mmol, 3 equiv) at room temperature under nitrogen atmosphere. The resulting mixture was stirred overnight at room temperature under nitrogen atmosphere. The reaction was quenched with water (300 mL) and the mixture was extracted with CH₂Cl₂ (3×300 mL). The combined organic layers were washed with brine (3×200 mL), dried over anhydrous Na₂SO₄, and concentrated under reduced pressure. The residue was purified by C18 column using water and acetonitrile as mobile phase. Fractions containing the desired compound were evaporated to dryness to give 25 g of NN-dibenzylbutanamide (INT-A16-1) as a white solid (82%). LCMS: m/z (EST), [M+H]⁺=268.00.

Step 2. Preparation of NN-dibenzyl-2-ethylpent-4-enamide (INT-A16-2). To a stirred mixture of NN-dibenzylbutanamide (10 g, 37.40 mmol, 1 equiv) in tetrahydrofuran (100 mL) were added LiHMDS (41.14 mL, 41.14 mmol, 1.1 equiv) at −78 °C under nitrogen atmosphere and allyl bromide (6.79 g, 56.10 mmol, 1.5 equiv). The resulting mixture was stirred for 2 h at −60 °C under nitrogen atmosphere and warmed to room temperarure. The reaction was quenched with water (200 mL) and the mixture was extracted with ethyl acetate (3×200 mL). The combined organic layers were washed with brine (3×200 mL), dried over anhydrous Na₂SO₄, and concentrated under reduced pressure. The residue was purified by C18 column using water and acetonitrile as mobile phase. Fractions containing the desired compound were evaporated to dryness to give 8 g of N,N-dibenzyl-2-ethylpent-4-enamide (INT-A16-2) as a colorless oil (69%). LCMS: m/z (ESI), [M+H]⁺=308.15. ¹H NMR (DMSO-d₆, 400 MHz) δ 0.75 (3H, t), 1.30-1.46 (1H, m), 1.46-1.63 (1H, m), 2.02-2.17 (1H, m), 2.21-2.31 (1H, m), 2.63-2.79 (1H, m), 4.49-4.61 (4H, m), 4.86-5.07 (2H, m), 5.59-5.83 (1H, m), 7.12-7.49 (10H, m).

Step 3. Preparation of N,N-dibenzyl-2-ethyl-5-hydroxypentanamide (INT-A16-3). A mixture of N,N-dibenzyl-2-ethylpent-4-enamide (6.0 g, 19.51 mmol, 1 equiv) and 9-borabicyclo[3.3.1]nonane (5.95 g, 48.79 mmol, 2.5 equiv) in THF (15 mL) was stirred for 1 h at room temperature under nitrogen atmosphere. Then NaOH (1.17 g, 29.27 mmol, 1.5 equiv) in H₂O (1.50 mL, 83.33 mmol, 4.27 equiv) and H₂O₂(12.12 mL, 156.13 mmol, 8.0 equiv, 30%) were added at 0 °C. The resulting mixture was stirred for 2 h at room temperature. The reaction was quenched with sat. Na₂S₂O₃ solution (100 mL) and the mixture was extracted with ethyl acetate (3×100 mL). The combined organic layers were washed with brine (3×100 mL), dried over anhydrous Na₂SO₄, and concentrated under reduced pressure. The residue was purified by C18 column using water and acetonitrile as mobile phase. Fractions containing the desired compound were evaporated to dryness to give 5.0 g of N,N-dibenzyl-2-ethyl-5-hydroxypentanamide (INT-A16-3) as a colorless oil (79%). LCMS: m/z (ESI), [M+H]⁺=326.15. ¹H NMR (DMSO-d₆, 400 MHz) δ 0.75 (3H, t), 1.30-1.56 (6H, m), 2.60 (1H, t), 3.23-3.31 (2H, m), 4.56 (4H, d), 7.05-7.54 (10H, m).

Step 4. Preparation of 4-[(dibenzylamino)methyl]hexan-1-ol (INT-A16-4). To a mixture of N,N-dibenzyl-2-ethyl-5-hydroxypentanamide (5.0 g, 15.36 mmol, 1 equiv) in tetrahydrofuran (50 mL) was added LiAlH₄ (1.16 g, 30.73 mmol, 2.0 equiv) at 0 °C under nitrogen atmosphere. The resulting mixture was stirred for 2 h at 0 °C. The reaction was quenched with water (1.2 mL) and NaOH (387.12 mg, 9.679 mmol, 0.63 equiv) in H₂O (800 mg) at room temperature. The resulting mixture was filtered. The filter cake was washed with tetrahydrofuran (2×50 mL). The organic solution was concentrated under reduced pressure. The residue was purified by C18 column using water and acetonitrile as mobile phase. Fractions containing the desired compound were evaporated to dryness to give 3.0 g of 4-[(dibenzylamino)methyl]hexan-1-ol (INT-A16-4) as a colorless oil (63%). LCMS: m/z (ESI), [M+H]⁺=312.15.

Step 5. Preparation of methyl 4-(aminomethyl)hexan-1-ol (INT-A16-5). A mixture of 4-[(dibenzylamino)methyl]hexan-1-ol (3.0 g, 9.63 mmol, 1 equiv) and Pd/C (0.31 g, 2.89 mmol, 0.3 equiv) in MeOH (50 mL) was stirred for 2 h at 20 °C under hydrogen atmosphere. The mixture was filtered. The filter cake was washed with methanol (300 mL). The solution was concentrated under reduced pressure to give 1.2 g of 4-(aminomethyl)hexan-1-ol (INT-A16-5) as a colorless oil. This material was used for the next reaction without further purification. ¹H NMR (DMSO-d₆, 400 MHz) δ 0.82 (3H, t), 1.0-1.44 (7H, m), 2.18-2.48 (2H, m), 3.37 (2H, t).

Step 6. Preparation of 4-{[(5-bromo-2-nitrophenyl)amino]methyl}hexan-1-ol (INT-A 16-6). A mixture of 4-(aminomethyl)hexan-1-ol (140 mg, 1.07 mmol, 1 equiv), 4-bromo-2-fluoro-1-nitrobenzene (234.72 mg, 1.07 mmol, 1.0 equiv), and K₂CO₃ (294.91 mg, 2.13 mmol, 2.0 equiv) in acetonitrile (5.0 mL) was stirred overnight at 60 °C under nitrogen atmosphere. The mixture was cooled to room temperature. The reaction was quenched with water (50 mL) and the mixture was extracted with ethyl acetate (3×50 mL). The combined organic layers were washed with brine (3×50 mL), dried over anhydrous Na₂SO₄, and concentrated under reduced pressure. The residue was purified by C18 column using water and acetonitrile as mobile phase. Fractions containing the desired compound were evaporated to dryness to give 300 mg of 4-{[(5-bromo-2-nitrophenyl)amino]methyl}hexan-1-ol (INT-A16-6) as a yellow solid (85%). LCMS: m/z (ESI), [M+H]⁺=330.90. ¹H NMR (DMSO-d₆, 400 MHz) δ 0.90 (3H, t), 1.34-1.49 (6H, m), 1.60-1.70 (1H, m), 3.28 (2H, t), 3.39 (2H, d), 4.32-4.43 (1H, m), 6.84 (1H, d), 7.26 (1H, d), 7.99 (1H, d), 8.15 (1H, t)

Step 7. Preparation of methyl 5-{5-[(4-{[(5-bromo-2-nitrophenyl)amino]methyl}hexyl)oxy]-1-methylpyrazol-4-yl}-1-methyl-6-oxopyridine-3-carboxylate (INT-A16-7). To a stirred mixture of methyl 5-(5-hydroxy-1-methylpyrazol-4-yl)-1-methyl-6-oxopyridine-3-carboxylate (M2, 262.29 mg, 0.99 mmol, 1.00 equiv) and triphenylphosphine (784.00 mg, 2.99 mmol, 3 equiv) in tetrahydrofuran (5 mL) were added diisopropylazodicarboxylate (604.41 mg, 2.99 mmol, 3 equiv) and 4-{[(5-bromo-2-nitrophenyl)amino]methyl}hexan-1-ol (330 mg, 0.99 mmol, 1.00 equiv) at 0 °C under nitrogen atmosphere. The resulting mixture was stirred for 2 h at 0 °C under nitrogen atmosphere. The reaction was quenched with water (50 mL) and the mixture was extracted with ethyl acetate (3×50 mL). The combined organic layers were washed with brine (3×50 mL), dried over anhydrous Na₂SO₄, and concentrated under reduced pressure. The residue was purified by C18 column using water and acetonitrile as mobile phase to give 500 mg of methyl 5-{5-[(4-{[(5-bromo-2-nitrophenyl)amino]methyl}hexyl)oxy]-1-methylpyrazol-4-yl}-1-methyl-6-oxopyridine-3-carboxylate (INT-A16-7) as an orange oil (87%). LCMS: m/z (ESI), [M+H]⁺=577.85.

Step 8. Preparation of methyl 5-{5-[(4-{[(2-amino-5-bromophenyl)amino]methyl}hexyl)oxy]-1-methylpyrazol-4-yl}-1-methyl-6-oxopyridine-3-carboxylate (INT-A16-8). A mixture of 5-{5-[(4-{[(5-bromo-2-nitrophenyl)amino]methyl}hexyl)oxy]-1-methylpyrazol-4-yl}-1-methyl-6-oxopyridine-3-carboxylate (400 mg, 0.69 mmol, 1 equiv), Raney Ni (297.25 mg, 3.47 mmol, 5 equiv), and hydrazine hydrate (98%) (52.11 mg, 1.04 mmol, 1.5 equiv) in methanol (5 mL) was stirred for 2 h at 0 °C under nitrogen atmosphere. The resulting mixture was filtered. The filter cake was washed with methanol (3×30 mL). The solution was concentrated under reduced pressure. The residue was purified by Prep-TLC eluted with dichloromethane/methanol (10/1) to give 300 mg of 5-{5-[(4-{[(2-amino-5-bromophenyl)amino]methyl}hexyl)oxy]-1-methylpyrazol-4-yl}-1-methyl-6-oxopyridine-3-carboxylate (INT-A16-8) as a brown solid (79%). LCMS: m/z (EST), [M+H]⁺=546.00.

Step 9. Preparation of methyl 5-[5-({4-[(2-amino-6-bromo-1,3-benzodiazol-1-yl)methyl]hexyl}oxy)-1-methylpyrazol-4-yl]-1-methyl-6-oxopyridine-3-carboxylate (INT-A 16-9). A mixture of methyl 5-{5-[(4-{[(2-amino-5-bromophenyl)amino]methyl}hexyl)oxy]-1-methylpyrazol-4-yl}-1-methyl-6-oxopyridine-3-carboxylate (300 mg, 0.55 mmol, 1 equiv) and BrCN (69.78 mg, 0.66 mmol, 1.2 equiv) in CH₂Cl₂ (5 mL) was stirred overnight at room temperature under nitrogen atmosphere and concentrated under reduced pressure. The residue was purified by Prep-TLC eluted with petroleum ether/ethyl acetate (1/3) to give 220 mg of methyl 5-[5-({4-[(2-amino-6-bromo-1,3-benzodiazol-1-yl)methyl]hexyl}oxy)-1-methylpyrazol-4-yl]-1-methyl-6-oxopyridine-3-carboxylate (INT-A16-9) as a brown solid (90%). LCMS: m/z (ESI), [M+H]⁺=572.95.

Step 10. Preparation of methyl 5-[5-({4-[(2-amino-6-bromo-1,3-benzodiazol-1-yl)methyl]hexyl}oxy)-1-methylpyrazol-4-yl]-1-methyl-6-oxopyridine-3-carboxylic acid (INT-A16-10). A mixture of methyl 5-[5-({4-[(2-amino-6-bromo-1,3-benzodiazol-1-yl)methyl]hexyl}oxy)-1-methylpyrazol-4-yl]-1-methyl-6-oxopyridine-3-carboxylate (170 mg, 0.23 mmol, 1 equiv) and LiOH—H₂O (24.96 mg, 0.55 mmol, 2.5 equiv) in tetrahydrofuran (10 mL) and H₂O (2.5 mL) was stirred for 2 h at 20 °C under nitrogen atmosphere. The resulting mixture was concentrated under reduced pressure. The residue was purified by C18 column using water and acetonitrile as mobile phase. Fractions containing the desired compound were evaporated to dryness to give 150 mg of 5-[5-({4-[(2-amino-6-bromo-1,3-benzodiazol-1-yl)methyl]hexyl}oxy)-1-methylpyrazol-4-yl]-1-methyl-6-oxopyridine-3-carboxylic acid (INT-A16-10) as an orange oil (87%). LCMS: m/z (ESI), [M+H]⁺=559.00.

Step 11. Preparation of methyl 16-bromo-11-ethyl-5,26-dimethyl-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (INT-A16-11). A mixture of 5-[5-({4-[(2-amino-6-bromo-1,3-benzodiazol-1-yl)methyl]hexyl}oxy)-1-methylpyrazol-4-yl]-1-methyl-6-oxopyridine-3-carboxylic acid (150 mg, 0.27 mmol, 1 equiv), N,N,N,N-tetramethyl-O-(7-azabenzotriazol-1-yl)uronium hexafluorophospate (204.63 mg, 0.54 mmol, 2.0 equiv) and N,N-diisopropylethylamine (104.33 mg, 0.81 mmol, 3.0 equiv) in dioxane (5 mL) was stirred for 2 h at 60 °C under nitrogen atmosphere. The mixture was cooled to room temperature. The reaction was quenched with water (30 mL) and the mixture was extracted with ethyl acetate (3×30 mL). The combined organic layers were washed with brine (3×30 mL), dried over anhydrous Na₂SO₄, and concentrated under reduced pressure. The residue was purified by Prep-TLC eluted with dichloromethane/methanol (5/1) to give 100 mg of 16-bromo-11-ethyl-5,26-dimethyl-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (INT-A16-11) as a light yellow solid (69%). LCMS: m/z (EST), [M+H]⁺=540.85.

Step 12. Preparation of methyl 5-{5-[(4-{[(2-amino-5-bromophenyl)amino]methyl}hexyl)oxy]-1-methylpyrazol-4-yl}-1-methyl-6-oxopyridine-3-carboxylate (A16). A mixture of 1-methylpiperazine (83.56 mg, 0.85 mmol, 5.0 equiv), 16-bromo-11-ethyl-5,26-dimethyl-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (90 mg, 0.17 mmol, 1.00 equiv), BrettPhos Pd G₃ (45.37 mg, 0.05 mmol, 0.3 equiv), and LiHMDS (1.00 mL, 1.00 mmol, 6.0 equiv) in dioxane (5.0 mL) was stirred for 2 h at 60 °C under nitrogen atmosphere. The mixture was cooled to room temperature. The reaction was quenched by the addition of sat. NH₄Cl solution (30 mL) and the mixture was extracted with ethyl acetate (3×30 mL). The combined organic layers were washed with brine (3×30 mL), dried over anhydrous Na₂SO₄, and concentrated under reduced pressure. The residue was purified by Prep-TLC eluted with dichloromethane/methanol (8/1) to give 2.0 mg of 11-ethyl-5,26-dimethyl-16-(4-methylpiperazin-1-yl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (A16) as a light yellow solid (2%). LCMS: m/z (ESI), [M+H]⁺=559.50. ¹H NMR (CD₃OD, 400 MHz) δ 0.82 (3H, t), 1.17-1.47 (6H, m), 1.74 (2H, s), 2.08 (1H, s), 2.28 (1H, s), 2.71 (1H, d), 2.88 (3H, s), 3.43 (4H, s), 3.68 (3H, s), 3.77 (3H, s), 3.80-3.92 (2H, m), 4.04 (1H, s), 4.30 (1H, d), 4.42 (1H, d), 6.99 (1H, s), 7.01 (1H, s), 7.39 (1H, d), 8.24 (1H, s), 8.40 (1H, s), 8.87 (1H, s)

Step 13. Preparation of 11-ethyl-5,26-dimethyl-16-(4-methylpiperazin-1-yl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione,isomer1 (EXAMPLE A16A) and isomer 2 (EXAMPLE A16B). The racemic mixture of 11-ethyl-5,26-dimethyl-16-(4-methylpiperazin-1-yl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (A16, 25 mg, 0.04 mmol) was separated by Prep-Chiral-HPLC with CHIRALPAK IG column (2*25 cm, 5 μm) using tert-butyl methyl ether containing 0.1% diethylamine as mobile phase A and EtOH/CH₂Cl₂ (1/1) as mobile phase B to give 1.8 mg of isomer 1 (EXAMPLE A16A, 7%) and 2.2 mg of isomer 2 (EXAMPLE A16B, 9%) as a white solid.

Isomer 1: LCMS: m/z (ESI), [M+H]⁺=559.45. ¹H NMR (CD₃OD, 400 MHz) δ 0.72-0.91 (3H, m), 1.16-1.45 (2H, m), 1.65-1.80 (2H, m), 2.03-2.12 (1H, m), 2.20-2.36 (1H, m), 2.60-2.74 (1H, m), 2.80 (4H, s), 3.15-3.26 (4H, m), 3.38 (4H, t), 3.56-3.71 (4H, m), 3.80 (3H, d), 4.00-4.06 (1H, m), 4.25-4.31 (1H, m), 4.39-4.45 (1H, m), 6.91 (1H, s), 7.02 (1H, d), 7.37 (1H, s), 8.12 (1H, s), 8.41 (1H, d), 8.83 (1H, s). Chiral-HPLC, Rt=4.108 min.

Isomer 2: LCMS: m/z (ESI), [M+H]⁺=559.35. ¹H NMR (CD₃OD, 400 MHz) δ 0.72-0.91 (3H, m), 1.16-1.45 (2H, m), 1.65-1.80 (2H, m), 2.03-2.12 (1H, m), 2.20-2.36 (1H, m), 2.60-2.74 (1H, m), 2.80 (4H, s), 3.15-3.26 (4H, m), 3.38 (4H, t), 3.56-3.71 (4H, m), 3.80 (3H, d), 4.00-4.06 (1H, m), 4.25-4.31 (1H, m), 4.39-4.45 (1H, m), 6.83 (1H, s), 6.98 (1H, d), 7.35 (1H, s), 8.05 (1H, s), 8.41 (1H, d), 8.77 (1H, s). Chiral-HPLC, Rt=5.613 min.

### Example A18

### 5′,26′-dimethyl-16′-(4-methylpiperazin-1-yl)-7′-oxa-4′,5′,13′,20′,22′,26′-hexaazaspiro[cyclopropane-1,11′-pentacyclo[22.3.1.0\hat{ }{2,6}.0\hat{ }{13,21}.0\hat{ }{14,19}]octacosan]-1′(28′),2′(6′),3′,14′,16′,18′,20′,24′-octaene-23′,27′-dione

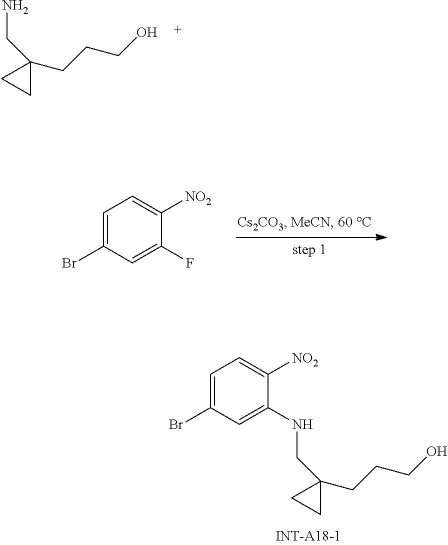 [SMILES: NCC1(CCCO)CC1 ; O=[N+]([O-])c1ccc(Br)cc1F ; O=[N+]([O-])c1ccc(Br)cc1NCC1(CCCO)CC1]

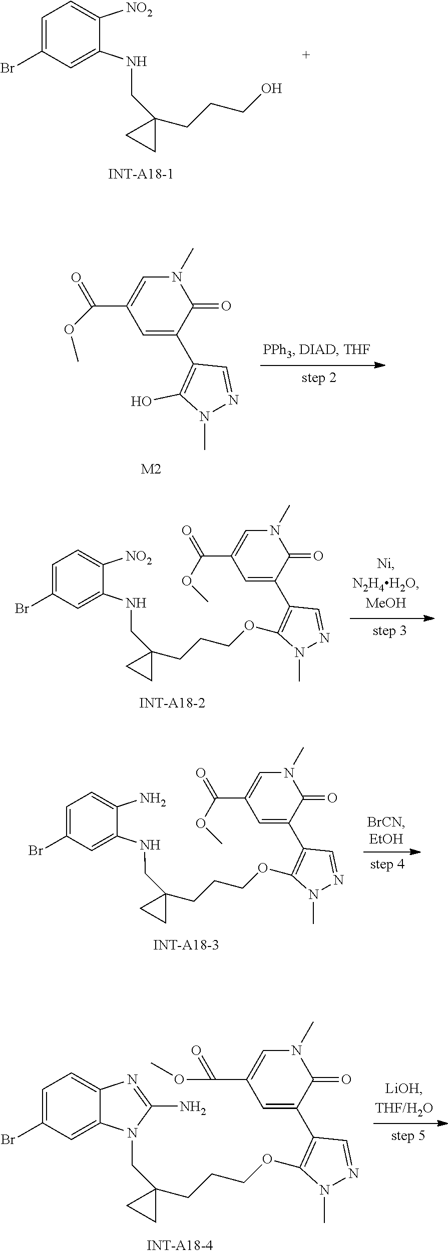 [SMILES: O=[N+]([O-])c1ccc(Br)cc1NCC1(CCCO)CC1 ; COC(=O)c1cc(-c2cnn(C)c2O)c(=O)n(C)c1 ; "M2" ; COC(=O)c1cc(-c2cnn(C)c2OCCCC2(CNc3cc(Br)ccc3[N+](=O)[O-])CC2)c(=O)n(C)c1 ; COC(=O)c1cc(-c2cnn(C)c2OCCCC2(CNc3cc(Br)ccc3N)CC2)c(=O)n(C)c1 ; COC(=O)c1cc(-c2cnn(C)c2OCCCC2(Cn3c(N)nc4ccc(Br)cc43)CC2)c(=O)n(C)c1]

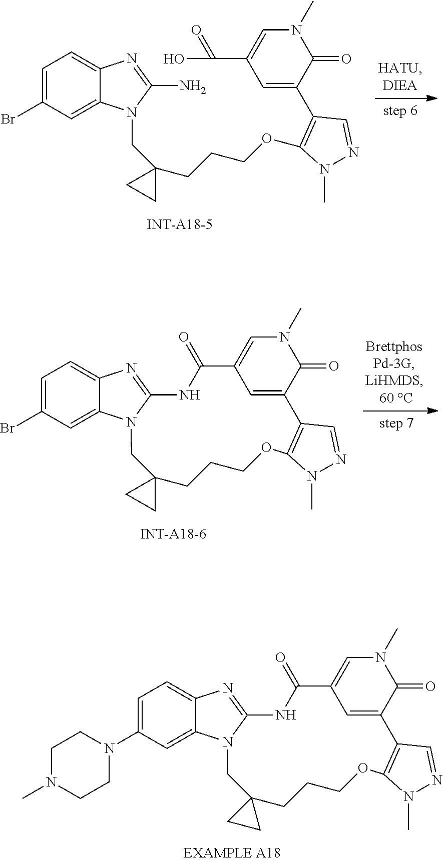 [SMILES: Cn1ncc(-c2cc(C(=O)O)cn(C)c2=O)c1OCCCC1(Cn2c(N)nc3ccc(Br)cc32)CC1 ; Cn1ncc2c1OCCCC1(CC1)Cn1c(nc3ccc(Br)cc31)NC(=O)c1cc-2c(=O)n(C)c1 ; CN1CCN(c2ccc3nc4n(c3c2)CC2(CCCOc3c(cnn3C)-c3cc(cn(C)c3=O)C(=O)N4)CC2)CC1]

Step 1. Preparation of 3-(1-(((5-bromo-2-nitrophenyl)amino)methyl)cyclopropyl)propan-1-ol (INT-A18-1). A mixture of 3-[1-(aminomethyl)cyclopropyl]propan-1-ol (500 mg, 3.87 mmol, 1 equiv), K₂CO₃ (1069 mg, 7.74 mmol, 2 equiv) and 4-bromo-2-fluoro-1-nitrobenzene (936 mg, 4.25 mmol, 1.1 equiv) in acetonitrile (5 mL) was stirred for 2 h at 60 °C under nitrogen atmosphere. The mixture was cooled to room temperature. The resulting mixture was concentrated under vacuum. The residue was loaded onto a silica gel column and eluted with petroleum ether/ethyl acetate (5/1) to give 1.0 g of 3-(1-{[(5-bromo-2-nitrophenyl)amino]methyl}cyclopropyl)propan-1-ol (INT-A18-1) as a colorless oil (78%). ¹H NMR (DMSO-d₆, 400 MHz) δ 0.37-0.55 (4H, m), 1.31-1.44 (2H, m), 1.45-1.60 (2H, m), 3.25 (2H, d), 3.36-3.39 (2H, m), 4.45 (1H, s), 6.78-6.89 (1H, m), 7.26 (1H, d), 8.00 (1H, d), 8.08 (1H, t)

Step 2. Preparation of methyl 5-(5-(3-(1-(((5-bromo-2-nitrophenyl)amino)methyl)cyclopropyl)propoxy)-1-methyl-1H-pyrazol-4-yl)-1-methyl-6-oxo-1,6-dihydropyridine-3-carboxylate (INT-A18-2). To a stirred mixture of methyl 5-(5-hydroxy-1-methylpyrazol-4-yl)-1-methyl-6-oxopyridine-3-carboxylate (M2, 700 mg, 2.66 mmol, 1 equiv) and triphenylphosphine (2092 mg, 7.97 mmol, 3 equiv) in tetrahydrofuran (10 mL) were added diisopropylazodicarboxylate (1613 mg, 7.97 mmol, 3 equiv) and 3-(1-{[(5-bromo-2-nitrophenyl)amino]methyl}cyclopropyl)propan-1-ol (875 mg, 2.66 mmol, 1 equiv) at 0 °C under nitrogen atmosphere. The mixture was stirred for 2 h at room teperature. The reaction was quenched with water (80 mL) and the mixture was extracted with ethyl acetate (3×60 mL). The combined organic layers were washed with brine (3×50 mL), dried over anhydrous Na₂SO₄, and concentrated under reduced pressure. The residue was loaded onto a silica gel column and eluted with petroleum ether/ethyl acetate (1/10) to give 700 mg of methyl 5-{5-[3-(1-{[(5-bromo-2-nitrophenyl)amino]methyl}cyclopropyl)propoxy]-1-methylpyrazol-4-yl}-1-methyl-6-oxopyridine-3-carboxylate (INT-A18-2) as a yellow oil (45%). ¹H NMR (DMSO-d₆, 400 MHz) δ 0.42-0.47 (m, 2H), 0.52 (2H, d), 1.50-1.58 (m, 2H), 1.80-1.88 (2H, m), 3.20-3.29 (2H, m), 3.57 (3H, s), 3.66 (3H, s), 3.77 (3H, s), 3.88-3.96 (2H, m), 6.79-6.84 (1H, m), 7.25 (1H, d), 7.55-7.66 (1H, m), 7.98 (1H, d), 8.07 (1H, d), 8.10 (1H, d), 8.42 (1H, d).

Step 3. Preparation of methyl 5-(5-(3-(1-(((2-amino-5-bromophenyl)amino)methyl)cyclopropyl)propoxy)-1-methyl-1H-pyrazol-4-yl)-1-methyl-6-oxo-1,6-dihydropyridine-3-carboxylate (INT-A18-3). To a stirred mixture of methyl 5-{5-[3-(1-{[(5-bromo-2-nitrophenyl)amino]methyl}cyclopropyl)propoxy]-1-methylpyrazol-4-yl}-1-methyl-6-oxopyridine-3-carboxylate (INT-A18-2, 700 mg, 1.22 mmol, 1 equiv) and Raney-Ni (50 mg) in methanol (10 mL) was added NH₂NH₂—H₂O (122 mg, 2.43 mmol, 2 equiv) in portions at 0 °C under nitrogen atmosphere. The mixture was stirred for 1 h at 0 °C. The resulting mixture was filtered. The filter cake was washed with methanol (3×30 mL) and the solution was concentrated under vacuum. The residue was used in the next step directly.

Step 4. Preparation of methyl 5-(5-(3-(1-((2-amino-6-bromo-1H-benzo[d]imidazol-1-yl)methyl)cyclopropyl)propoxy)-1-methyl-1H-pyrazol-4-yl)-1-methyl-6-oxo-1,6-dihydropyridine-3-carboxylate (INT-A18-4). A mixture of methyl 5-{5-[3-(1-{[(2-amino-5-bromophenyl)amino]methyl}cyclopropyl)propoxy]-1-methylpyrazol-4-yl}-1-methyl-6-oxopyridine-3-carboxylate (580 mg, 1.06 mmol, 1 equiv) and BrCN (135 mg, 1.27 mmol, 1.2 equiv) in ethyl alcohol (6 mL) was stirred for 2 h at room temperature under nitrogen atmosphere. The mixture was concentrated under vacuum. The residue was loaded onto a silica gel column and eluted with methylene chloride/methanol (12/1) to give 600 mg of methyl 5-[5-(3-{1-[(2-amino-6-bromo-1,3-benzodiazol-1-yl)methyl]cyclopropyl}propoxy)-1-methylpyrazol-4-yl]-1-methyl-6-oxopyridine-3-carboxylate (INT-A18-4) as a brown solid (98%). ¹H NMR (DMSO-d₆, 400 MHz) δ 0.36 (2H, t), 0.45-0.54 (2H, m), 1.40-1.50 (2H, m), 1.78-1.88 (2H, m), 3.58 (3H, s), 3.65 (3H, s), 3.74 (3H, s), 3.88 (2H, t), 4.08 (2H, s), 6.65 (2H, s), 7.06 (2H, s), 7.35 (1H, s), 7.97 (1H, s), 8.08 (1H, d), 8.43 (1H, d)

Step 5. Preparation of 5-(5-(3-(1-((2-amino-6-bromo-1H-benzo[d]imidazol-1-yl)methyl)cyclopropyl)propoxy)-1-methyl-1H-pyrazol-4-yl)-1-methyl-6-oxo-1,6-dihydropyridine-3-carboxylic acid (INT-A18-5). A mixture of methyl 5-[5-(3-{1-[(2-amino-6-bromo-1,3-benzodiazol-1-yl)methyl]cyclopropyl}propoxy)-1-methylpyrazol-4-yl]-1-methyl-6-oxopyridine-3-carboxylate (580 mg, 1.02 mmol, 1 equiv) and LiOH·H₂O (85 mg, 2.03 mmol, 2 equiv) in tetrahydrofuran/H₂O (0.8 mL/0.2 mL) was stirred for 2 h at room temperature under nitrogen atmosphere. The mixture was concentrated under vacuum. The residue was purified by C18 column using water and acetonitrile as mobile phase. Fractions containing the desired compound were evaporated to dryness to give 400 mg of 5-[5-(3-{1-[(2-amino-6-bromo-1,3-benzodiazol-1-yl)methyl]cyclopropyl}propoxy)-1-methylpyrazol-4-yl]-1-methyl-6-oxopyridine-3-carboxylic acid (INT-A18-5) as a brown solid (70%). ¹H NMR (DMSO-d₆, 400 MHz) δ 0.24-0.35 (2H, m), 0.42 (2H, t), 1.40-1.59 (2H, m), 1.89-1.96 (2H, m), 3.51 (3H, s), 3.63 (3H, s), 3.90 (2H, t), 4.17 (2H, s), 6.94 (2H, s), 7.02 (2H, d), 7.33-7.37 (1H, m), 7.94 (1H, s), 8.07 (1H, s), 8.38 (1H, d).

Step 6. Preparation of 16′-bromo-5′,26′-dimethyl-7′-oxa-4′,5′,13′,20′,22′,26′-hexaazaspiro[cyclopropane-1,11′-pentacyclo[22.3.1.0\hat{ }{2,6}.0\hat{ }{13,21}.0\hat{ }{14,19}]octacosan]-1′(28′),2′(6′),3′,14′,16′,18′,20′,24′-octaene-23′,27′-dione (INT-A18-6). To a mixture of 5-[5-(3-{1-[(2-amino-6-bromo-1,3-benzodiazol-1-yl)methyl]cyclopropyl}propoxy)-1-methylpyrazol-4-yl]-1-methyl-6-oxopyridine-3-carboxylic acid (330 mg, 0.59 mmol, 1 equiv) and N,N,N,N-tetramethyl-O-(7-azabenzotriazol-1-yl)uronium hexafluorophospate (338 mg, 0.89 mmol, 1.5 equiv) in dioxane (5 mL) was added N,N-diisopropylethylamine (230 mg, 1.78 mmol, 3 equiv) at room temperature under nitrogen atmosphere. The mixture was stirred for 3 h at that temperature. The reaction was quenched with water (80 mL) and the mixture was extracted with ethyl acetate (3×60 mL). The combined organic layers were washed with brine (3×50 mL), dried over anhydrous Na₂SO₄, and concentrated under reduced pressure. The residue was purified by Prep-TLC and eluted with methylene dichloride/methanol (12/1) to give 300 mg of 16′-bromo-5′,26′-dimethyl-7′-oxa-4′,5′,13′,20′,22′,26′-hexaazaspiro[cyclopropane-1,11′-pentacyclo[22.3.1.0\hat{ }{2,6}.0\hat{ }{13,21}.0\hat{ }{14,19}]octacosan]-1′(28′),2′(6′),3′,14′,16′,18′,20′,24′-octaene-23′,27′-dione (INT-A18-6) as a brown solid (93%). ¹H NMR (DMSO-d₆, 400 MHz) δ 0.39 (2H, t), 0.63 (2H, t), 1.78 (2H, s), 2.20-2.28 (2H, m), 3.10-3.18 (2H, m), 3.65 (3H, d), 3.73 (3H, s), 4.15-4.28 (2H, m), 7.34-7.51 (2H, m), 7.75 (1H, d), 8.24-8.36 (2H, m), 8.71 (1H, d), 12.82 (1H, s)

Step 7. Preparation of 5′,26′-dimethyl-16′-(4-methylpiperazin-1-yl)-7′-oxa-4′,5′,13′,20′,22′,26′-hexaazaspiro[cyclopropane-1,11′-pentacyclo[22.3.1.0\hat{ }{2,6}.0\hat{ }{13,21}.0\hat{ }{14,19}]octacosan]-1′(28′),2′(6′),3′,14′,16′,18′,20′,24′-octaene-23′,27′-dione (Example A18). To a stirred mixture of 16′-bromo-5′,26′-dimethyl-7′-oxa-4′,5′,13′,20′,22′,26′-hexaazaspiro[cyclopropane-1,11′-pentacyclo[22.3.1.0\hat{ }{2,6}.0\hat{ }{13,21}.0\hat{ }{14,19}]octacosan]-1′(28′),2′(6′),3′,14′,16′,18′,20′,24′-octaene-23′,27′-dione (100 mg, 0.18 mmol, 1 equiv), 1-methyl piperazine (74 mg, 0.74 mmol, 4.0 equiv), and BrettPhos Pd G₃ (50 mg, 0.05 mmol, 0.3 equiv) in 1,4-dioxane (4 mL) was added LiHMDS (1.12 mL, 1.12 mmol, 6 equiv) at room temperature under nitrogen atmosphere. The mixture was stirred for 1 h at 60 °C and then cooled to room temperature. The reaction was quenched with sat. NH₄Cl (aq.) solution (20 mL) and the mixture was extracted with ethyl acetate (3×20 mL). The combined organic layers were washed with brine (3×30 mL), dried over anhydrous Na₂SO₄, and concentrated under reduced pressure. The residue was purified by Prep-TLC and eluted with methylene dichloride/methanol (12/1) to give 18 mg of 5′,26′-dimethyl-16′-(4-methylpiperazin-1-yl)-7′-oxa-4′,5′,13′,20′,22′,26′-hexaazaspiro[cyclopropane-1,11′-pentacyclo[22.3.1.0\hat{ }{2,6}.0\hat{ }{13,21}.0\hat{ }{14,19}]octacosan]-1′(28′),2′(6′),3′,14′,16′,18′,20′,24′-octaene-23′,27′-dione (Example A18) as a white solid (17%). LCMS: m/z (EST), [M+H]⁺=557.35. ¹H NMR (DMSO-d₆, 400 MHz) δ 0.35 (2H, t), 0.62 (2H, t), 1.73-1.81 (2H, m), 2.24 (5H, s), 2.45-2.49 (4H, m), 3.10-3.17 (4H, m), 3.62 (3H, s), 3.72 (3H, s), 4.18-4.29 (4H, m), 6.75-6.89 (1H, m), 6.95 (1H, d), 7.36 (1H, d), 8.29 (2H, d), 8.72 (1H, d), 12.51 (1H, s).

### Example A19

### 5,12,26-Trimethyl-16-(4-methylpiperazin-1-yl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione

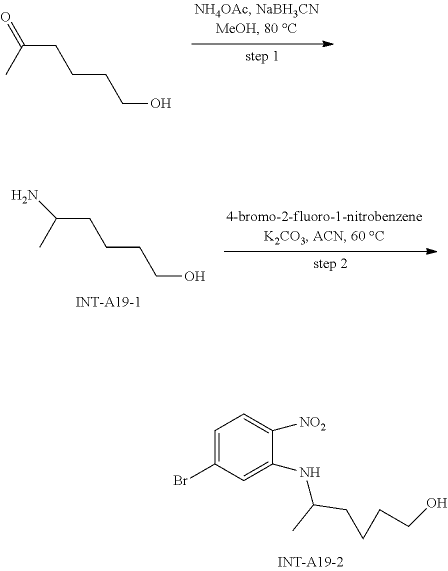

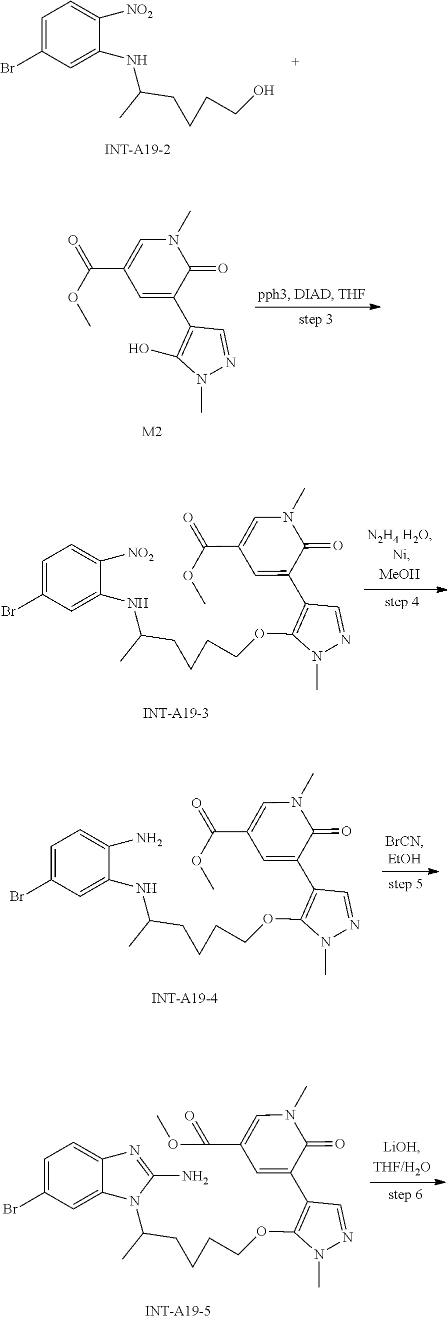 [SMILES: CC(CCCCO)Nc1cc(Br)ccc1[N+](=O)[O-] ; COC(=O)c1cc(-c2cnn(C)c2O)c(=O)n(C)c1 ; "M2" ; COC(=O)c1cc(-c2cnn(C)c2OCCCCC(C)Nc2cc(Br)ccc2[N+](=O)[O-])c(=O)n(C)c1 ; COC(=O)c1cc(-c2cnn(C)c2OCCCCC(C)Nc2cc(Br)ccc2N)c(=O)n(C)c1 ; COC(=O)c1cc(-c2cnn(C)c2OCCCCC(C)n2c(N)nc3ccc(Br)cc32)c(=O)n(C)c1]

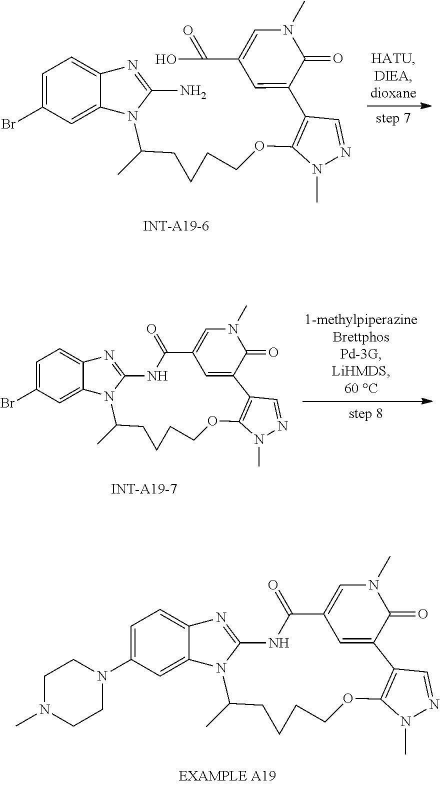 [SMILES: CC(CCCCOc1c(-c2cc(C(=O)O)cn(C)c2=O)cnn1C)n1c(N)nc2ccc(Br)cc21 ; CC1CCCCOc2c(cnn2C)-c2cc(cn(C)c2=O)C(=O)Nc2nc3ccc(Br)cc3n21 ; CC1CCCCOc2c(cnn2C)-c2cc(cn(C)c2=O)C(=O)Nc2nc3ccc(N4CCN(C)CC4)cc3n21]

Step 1. Preparation of 5-aminohexan-1-ol (INT-A19-1). A mixture of 6-hydroxy-hexan-2-one (1.0 g, 8.60 mmol, 1 equiv), NH₄OAc (3.32 g, 43.04 mmol, 5.0 equiv) and NaBH₃CN (1.35 g, 21.52 mmol, 2.5 equiv) in methanol (10 mL) was stirred for 2 h at 80 °C under nitrogen atmosphere. The mixture was cooled to room temperature. The reaction was quenched with water (200 mL) and the mixture was extracted with ethyl acetate (3×200 mL). The combined organic layers were washed with brine (3×200 mL), dried over anhydrous Na₂SO₄, and concentrated under reduced pressure. The residue was purified by C18 FLASH using water containing 10 mmol/L NH₄HCO₃ and 0.1% NH₃·H₂O and acetonitrile as mobile phase. Fractions containing the desired compound were evaporated to dryness to give 2.5 g of 5-aminohexan-1-ol (INT-A19-1) as a colorless oil (74%). LCMS: m/z (ESI), [M+H]⁺=118.15.

Step 2. Preparation of 5-((5-bromo-2-nitrophenyl)amino)hexan-1-ol (INT-A19-2). A mixture of 5-aminohexan-1-ol (2.5 g, 21.33 mmol, 1 equiv), K₂CO₃ (8.84 g, 63.99 mmol, 3 equiv) and 4-bromo-2-fluoro-1-nitrobenzene (14.08 g, 63.99 mmol, 3 equiv) in acetonitrile (80 mL) was stirred overnight at 60 °C under nitrogen atmosphere. The mixture was cooled to room temperature. The reaction was quenched with water (100 mL) and the mixture was extracted with ethyl acetate (3×100 mL). The combined organic layers were washed with brine (3×100 mL), dried over anhydrous Na₂SO₄, and concentrated under reduced pressure. The residue was purified on a silica gel column eluted with petroleum ether/ethyl acetate (5/1) to give 3.1 g of 5-[(5-bromo-2-nitrophenyl)amino]hexan-1-ol (INT-A19-2) as a yellow oil (42%). LCMS: m/z (ESI), [M+H]⁺=319.05. ¹H NMR (DMSO-d₆, 400 MHz) δ 1.18-1.26 (3H, m), 1.29-1.49 (4H, m), 1.50-1.68 (2H, m), 3.36-3.43 (2H, m), 3.81-3.90 (1H, m), 4.37 (1H, t), 6.78-6.86 (1H, m), 7.31 (1H, d), 7.94 (1H, d), 7.99 (1H, d).

Step 3. Preparation of methyl 5-(5-((5-((5-bromo-2-nitrophenyl)amino)hexyl)oxy)-1-methyl-1H-pyrazol-4-yl)-1-methyl-6-oxo-1,6-dihydropyridine-3-carboxylate (INT-A19-3). To a stirred mixture of methyl 5-(5-hydroxy-1-methylpyrazol-4-yl)-1-methyl-6-oxopyridine-3-carboxylate (M2, 500 mg, 1.89 mmol, 1.00 equiv) and triphenylphosphine (1494 mg, 5.69 mmol, 3 equiv) in tetrahydrofuran (20 mL) were added diisopropyl azodicarboxylate (1152 mg, 5.69 mmol, 3 equiv) and 5-[(5-bromo-2-nitrophenyl)amino]hexan-1-ol (662 mg, 2.08 mmol, 1.1 equiv) in tetrahydrofuran (10 mL) at 0 °C under nitrogen atmosphere. The mixture was stirred at 0 °C for 2 h. The reaction was quenched with water (50 mL) and the mixture was extracted with ethyl acetate (3×50 mL). The combined organic layers were washed with brine (3×100 mL), dried over anhydrous Na₂SO₄, and concentrated under reduced pressure. The residue was purified on a silica gel column eluted with petroleum ether/ethyl acetate (1/1) to give 510 mg of methyl 5-[5-({5-[(5-bromo-2-nitrophenyl)amino]hexyl}oxy)-1-methylpyrazol-4-yl]-1-methyl-6-oxopyridine-3-carboxylate (INT-A19-3) as a yellow solid (47%). LCMS: m/z (ESI), [M+H]⁺=564.05.

Step 4. Preparation of methyl 5-(5-((5-((2-amino-5-bromophenyl)amino)hexyl)oxy)-1-methyl-1H-pyrazol-4-yl)-1-methyl-6-oxo-1,6-dihydropyridine-3-carboxylate (INT-A19-4). To a stirred mixture of methyl 5-[5-({5-[(5-bromo-2-nitrophenyl)amino]hexyl}oxy)-1-methylpyrazol-4-yl]-1-methyl-6-oxopyridine-3-carboxylate (490 mg, 0.87 mmol, 1 equiv) and Raney Nickel (599 mg, 6.96 mmol, 8 equiv) in methanol (5 mL) was added hydrazine hydrate (87.23 mg, 1.742 mmol, 2.0 equiv) at 0 °C. The mixture was stirred for 2 h at 0 °C under nitrogen atmosphere. The mixture was filtered. The filter cake was washed with methanol (3×50 mL) and the resulting solution was concentrated under reduced pressure to give 400 mg of methyl 5-[5-({5-[(2-amino-5-bromophenyl)amino]hexyl}oxy)-1-methylpyrazol-4-yl]-1-methyl-6-oxopyridine-3-carboxylate (INT-A19-4) as a brown solid. This material was used for the next reaction without further purification. LCMS: m/z (ESI), [M+H]⁺=534.20.

Step 5. Preparation of methyl 5-(5-((5-(2-amino-6-bromo-1H-benzo[d]imidazol-1-yl)hexyl)oxy)-1-methyl-1H-pyrazol-4-yl)-1-methyl-6-oxo-1,6-dihydropyridine-3-carboxylate (INT-A19-5). A mixture of methyl 5-[5-({5-[(2-amino-5-bromophenyl)amino]hexyl}oxy)-1-methylpyrazol-4-yl]-1-methyl-6-oxopyridine-3-carboxylate (400 mg, 0.751 mmol, 1 equiv) and BrCN (95.49 mg, 0.901 mmol, 1.2 equiv) in ethanol (5 mL) was stirred overnight at room temperature under nitrogen atmosphere. The reaction was quenched with water (20 mL) and the mixture was extracted with dichloromethane (3×30 mL). The combined organic layers were washed with brine (3×50 mL), dried over anhydrous Na₂SO₄, and concentrated under reduced pressure. The residue was purified on a Prep-TLC eluted with dichloromethane/methanol (12/1) to give 320 mg of methyl 5-(5-{[5-(2-amino-6-bromo-1,3-benzodiazol-1-yl)hexyl]oxy}-1-methylpyrazol-4-yl)-1-methyl-6-oxopyridine-3-carboxylate (INT-A19-5) as a brown solid (76%). LCMS: m/z (ESI), [M+H]⁺=557.20.

Step 6. Preparation of 5-(5-((5-(2-amino-6-bromo-1H-benzo[d]imidazol-1-yl)hexyl)oxy)-1-methyl-1H-pyrazol-4-yl)-1-methyl-6-oxo-1,6-dihydropyridine-3-carboxylic acid (INT-A19-6). A mixture of methyl 5-(5-{[5-(2-amino-6-bromo-1,3-benzodiazol-1-yl)hexyl]oxy}-1-methylpyrazol-4-yl)-1-methyl-6-oxopyridine-3-carboxylate (300 mg, 0.54 mmol, 1 equiv) and LiOH·H₂O (67 mg, 1.61 mmol, 3.0 equiv) in tetrahydrofuran (4 mL) and water (1 mL) was stirred for 2 h at room temperature under nitrogen atmosphere. The mixture was concentrated under reduced pressure. The residue was purified by C18 FLASH using water containing 10 mmol/L NH₄HCO₃ and 0.1% NH₃·H₂O and acetonitrile as mobile phase. Fractions containing the desired compound were evaporated to dryness to give 260 mg of 5-(5-{[5-(2-amino-6-bromo-1,3-benzodiazol-1-yl)hexyl]oxy}-1-methylpyrazol-4-yl)-1-methyl-6-oxopyridine-3-carboxylic acid (INT-A19-6) as a brown solid (88%). LCMS: m/z (ESI), [M+H]⁺=545.20. ¹H NMR (DMSO-d₆, 400 MHz) δ 1.37-1.39 (3H, m), 1.60-1.84 (1H, m), 1.90-1.97 (1H, m), 3.51 (3H, s), 3.61 (3H, s), 3.80-3.83 (1H, m), 3.89-3.92 (1H, m), 4.69-4.73 (1H, m), 7.02-7.04 (2H, m), 7.11 (2H, s), 7.37 (1H, t), 8.01 (2H, d), 8.34 (1H, d)

Step 7. Preparation of 16-bromo-5,12,26-trimethyl-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (INT-A19-7). A mixture of 5-(5-{[5-(2-amino-6-bromo-1,3-benzodiazol-1-yl)hexyl]oxy}-1-methylpyrazol-4-yl)-1-methyl-6-oxopyridine-3-carboxylic acid (250 mg, 0.46 mmol, 1 equiv), N,N,N,N-tetramethyl-O-(7-azabenzotriazol-1-yl)uroniumhexafluorophospate (262 mg, 0.69 mmol, 1.5 equiv), and N,N-diisopropylethylamine (178 mg, 1.38 mmol, 3.0 equiv) in 1,4-dioxane (5 mL) was stirred overnight at room temperature under nitrogen atmosphere. The reaction was quenched with water (20 mL) and the mixture was extracted with dichloromethane (3×30 mL). The combined organic layers were washed with brine (3×50 mL), dried over anhydrous Na₂SO₄, and concentrated under reduced pressure. The residue was purified on a Prep-TLC eluted with dichloromethane/methanol (12/1) to give 170 mg of 16-bromo-5,12,26-trimethyl-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (INT-A19-7) as a brown solid (70%). LCMS: m/z (ESI), [M+H]⁺=525.15.

Step 8. Preparation of 5,12,26-trimethyl-16-(4-methylpiperazin-1-yl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (EXAMPLE A19). To a stirred mixture of 16-bromo-5,12,26-trimethyl-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (100 mg, 0.19 mmol, 1 equiv), 1-methylpiperazine (76 mg, 0.76 mmol, 4 equiv) and BrettPhos Pd G3 (51 mg, 0.06 mmol, 0.3 equiv) in 1,4-dioxane (4 mL) was added LiHMDS (4 mL, 1.14 mmol, 6 equiv) at room temperature under nitrogen atmosphere. The mixture was stirred for 1 h at 60 °C and cooled to room temperature. The reaction was quenched with sat.NH₄Cl (aq.) solution (10 mL) and the mixture was extracted with CH₂Cl₂ (3×30 mL). The combined organic layers were dried over anhydrous Na₂SO₄ and concentrated under reduced pressure. The residue was purified by prep-HPLC with XBridge Shield RP18 OBD column using water 10 mmol/L NH₄HCO₃ and 0.1% NH₃·H₂O and acetonitrile as mobile phase. Fractions containing the desired compound were evaporated to dryness to give 11 mg of 5,12,26-trimethyl-16-(4-methylpiperazin-1-yl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (EXAMPLE A19) as a white solid (10%). LCMS: m/z (ESI), [M+H]⁺=545.25. ¹H NMR (DMSO-d₆, 400 MHz) δ 1.54-1.62 (4H, m), 1.63-1.70 (1H, m), 1.73-1.79 (1H, m), 1.82-1.92 (1H, m), 2.12-2.20 (2H, m), 2.22-2.31 (1H, m), 2.38-2.45 (4H, m), 3.12-3.27 (4H, m), 3.31 (3H, s), 3.61 (3H, s), 3.71 (3H, s), 3.90-4.08 (1H, m), 4.23-4.27 (1H, m), 4.82-4.98 (1H, m), 6.87-6.86 (1H, m), 7.09 (1H, s), 7.35-7.32 (1H, m), 8.23-8.31 (2H, m), 8.89-8.86 (1H, m), 12.29 (1H, s).

### Example A20

### (11R)-11,26-Dimethyl-16-(4-methylpiperazin-1-yl)-5-(2,2,2-trifluoroethyl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione

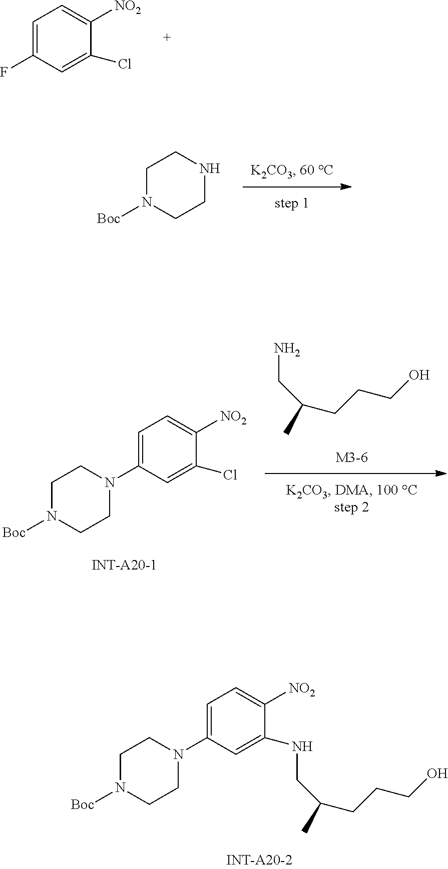

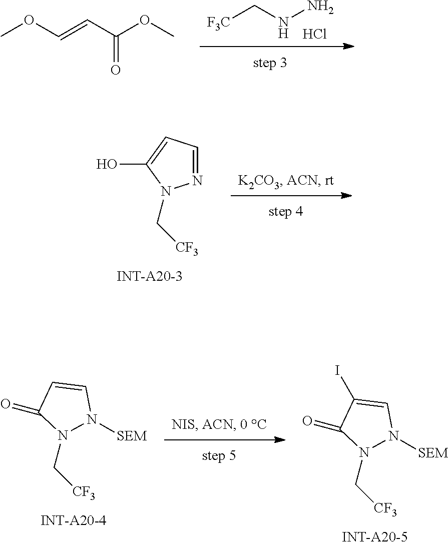

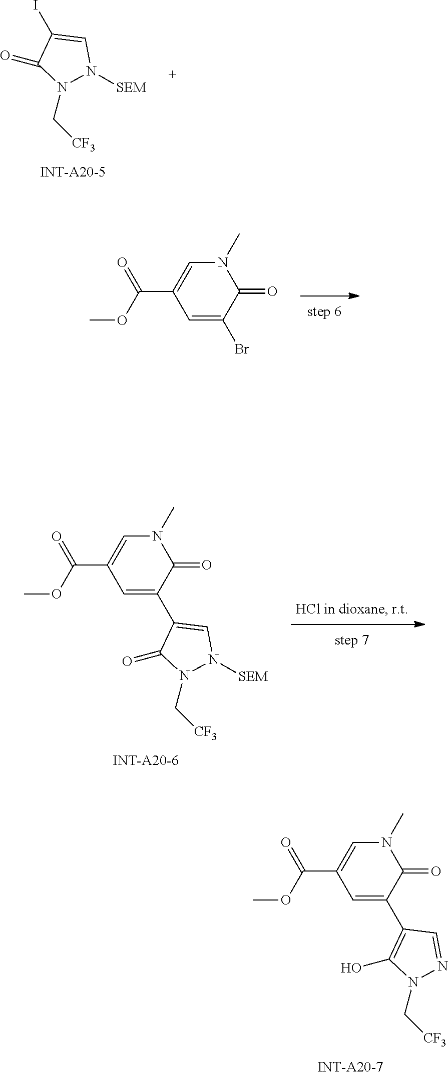 [SMILES: C[Si](C)(C)CCOCn1cc(I)c(=O)n1CC(F)(F)F ; COC(=O)c1cc(Br)c(=O)n(C)c1 ; COC(=O)c1cc(-c2cn(COCC[Si](C)(C)C)n(CC(F)(F)F)c2=O)c(=O)n(C)c1 ; COC(=O)c1cc(-c2cnn(CC(F)(F)F)c2O)c(=O)n(C)c1]

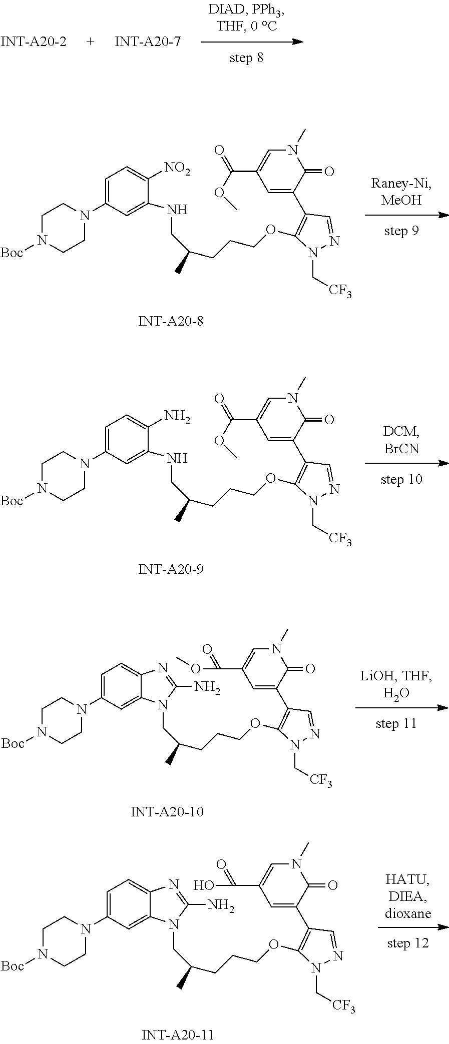 [SMILES: COC(=O)c1cc(-c2cnn(CC(F)(F)F)c2OCCC[C@@H](C)CNc2cc(N3CCN(C(=O)OC(C)(C)C)CC3)ccc2[N+](=O)[O-])c(=O)n(C)c1 ; COC(=O)c1cc(-c2cnn(CC(F)(F)F)c2OCCC[C@@H](C)CNc2cc(N3CCN(C(=O)OC(C)(C)C)CC3)ccc2N)c(=O)n(C)c1 ; COC(=O)c1cc(-c2cnn(CC(F)(F)F)c2OCCC[C@@H](C)Cn2c(N)nc3ccc(N4CCN(C(=O)OC(C)(C)C)CC4)cc32)c(=O)n(C)c1 ; C[C@H](CCCOc1c(-c2cc(C(=O)O)cn(C)c2=O)cnn1CC(F)(F)F)Cn1c(N)nc2ccc(N3CCN(C(=O)OC(C)(C)C)CC3)cc21]

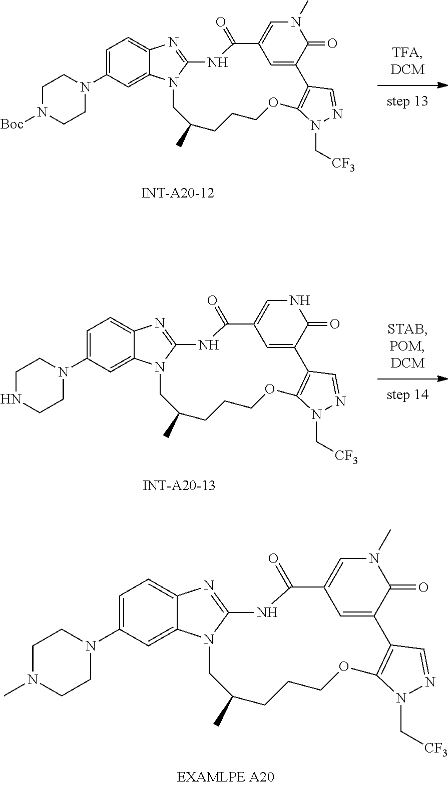 [SMILES: C[C@@H]1CCCOc2c(cnn2CC(F)(F)F)-c2cc(cn(C)c2=O)C(=O)Nc2nc3ccc(N4CCN(C(=O)OC(C)(C)C)CC4)cc3n2C1 ; C[C@@H]1CCCOc2c(cnn2CC(F)(F)F)-c2cc(c[nH]c2=O)C(=O)Nc2nc3ccc(N4CCNCC4)cc3n2C1 ; C[C@@H]1CCCOc2c(cnn2CC(F)(F)F)-c2cc(cn(C)c2=O)C(=O)Nc2nc3ccc(N4CCN(C)CC4)cc3n2C1]

Step 1. Preparation of tert-butyl 4-(3-chloro-4-nitrophenyl)piperazine-1-carboxylate (INT-A20-1). A mixture of 2-chloro-4-fluoro-1-nitrobenzene (10 g, 56.967 mmol, 1 equiv), K₂CO₃ (15.75 g, 113.934 mmol, 2 equiv), and tert-butyl piperazine-1-carboxylate (11.67 g, 62.664 mmol, 1.1 equiv) in acetonitrile (200 mL) was stirred overnight at 60 °C under nitrogen atmosphere. The mixture was cooled to room temperature. The reaction was quenched with water (800 mL) and the mixture was extracted with ethyl acetate (3×600 mL). The combined organic layers were washed with brine (3×500 mL), dried over anhydrous Na₂SO₄, and concentrated under reduced pressure. The residue was purified by silica gel chromatography eluted with petroleum ether/ethyl acetate (1/1) to afford 5.4 g of tert-butyl 4-(3-chloro-4-nitrophenyl)piperazine-1-carboxylate (INT-A20-1) as a yellow solid (27%). LCMS: m/z (EST), [M+H]⁺=342.10.

Step 2. Preparation of tert-butyl (R)-4-(3-((5-hydroxy-2-methylpentyl)amino)-4-nitrophenyl)piperazine-1-carboxylate (INT-A20-2). A mixture of tert-butyl 4-(3-chloro-4-nitrophenyl)piperazine-1-carboxylate (1.4 g, 4.096 mmol, 1 equiv), K₂CO₃ (1132.20 mg, 8.192 mmol, 2 equiv) and (4R)-5-amino-4-methylpentan-1-ol (M3-6, 480.03 mg, 4.096 mmol, 1 equiv) in N,N-dimethylacetamide (15 mL) was stirred for overnight at 100 °C under nitrogen atmosphere. The mixture was cooled to room temperature. The reaction was quenched with water (100 mL) and the mixture was extracted with ethyl acetate (3×100 mL). The combined organic layers were washed with brine (3×100 mL), dried over anhydrous Na₂SO₄, and concentrated under reduced pressure. The residue was purified by Prep-TLC eluted with petroleum ether/ethyl acetate (1/1) to afford 730 mg of tert-butyl 4-(3-{[(2R)-5-hydroxy-2-methylpentyl]amino}-4-nitrophenyl)piperazine-1-carboxylate (INT-A20-2) as a yellow solid (42%). LCMS: m/z (ESI), [M+H]⁺=423.25.

Step 3. Preparation of 2-(2,2,2-trifluoroethyl)pyrazol-3-ol (INT-A20-3). A mixture of methyl (2E)-3-methoxyprop-2-enoate (5 g, 43.06 mmol, 1 equiv) and (2,2,2-trifluoroethyl)hydrazine HCl salt (7.37 g, 64.59 mmol, 1.5 equiv) in methanol (20 mL) was stirred for 2 h at 80 °C under nitrogen atmosphere. The mixture was cooled to room temperature. The reaction was quenched with water (200 mL) and the mixture was extracted with dichloromethane (3×200 mL). The combined organic layers were washed with brine (3×200 mL), dried over anhydrous Na₂SO₄, and concentrated under reduced pressure. The residue was purified by silica gel column eluted with dichloromethane/methanol (20/1) to give 6 g of 2-(2,2,2-trifluoroethyl)pyrazol-3-ol (INT-A20-3) as a yellow solid (83%). ¹H NMR (DMSO, 400 MHz) δ 4.71 (2H, q), 5.38 (1H, d), 7.25 (1H, s), 11.40 (1H, s)

Step 4. Preparation of 2-(2,2,2-trifluoroethyl)-1-{[2-(trimethylsilyl)ethoxy]methyl}pyrazol-3-one (INT-A20-4). To a stirred mixture of 2-(2,2,2-trifluoroethyl)pyrazol-3-ol (6 g, 36.12 mmol, 1 equiv) and K₂CO₃ (9.98 g, 72.24 mmol, 2 equiv) in acetonitrile (100 mL) was added SEM-Cl (12.04 g, 72.24 mmol, 2 equiv) at 0 °C. The mixture was stirred for 2 h at 0 °C under nitrogen atmosphere. The reaction was quenched with water (200 mL) and the mixture was extracted with dichloromethane (3×200 mL). The combined organic layers were washed with brine (3×200 mL), dried over anhydrous Na₂SO₄, and concentrated under reduced pressure. The residue was purified by silica gel column eluted with petroleum ether/ethyl acetate (1/3) to give 900 mg of 2-(2,2,2-trifluoroethyl)-1-{[2-(trimethylsilyl)ethoxy]methyl}pyrazol-3-one (INT-A20-4) as a brown solid (8%). ¹H NMR (DMSO, 400 MHz) δ 0.81 (2H, d), 1.06 (1H, t), 1.24 (1H, s), 3.18 (1H, d), 3.45 (2H, m), 4.64 (2H, q), 5.09 (2H, s), 5.44 (1H, d), 8.08 (1H, d)

Step 5. Preparation of 4-iodo-2-(2,2,2-trifluoroethyl)-1-{[2-(trimethylsilyl)ethoxy]methyl}pyrazol-3-one (INT-A20-5). To a mixture of 2-(2,2,2-trifluoroethyl)-1-{[2-(trimethylsilyl)ethoxy]methyl}pyrazol-3-one (250 mg, 0.84 mmol, 1 equiv) in acetonitrile (5 mL) was added NIS (227 mg, 1.01 mmol, 1.2 equiv). The resulting mixture was stirred for 2 h at 0 °C under nitrogen atmosphere. The reaction was quenched with sat. sodium hyposulfite solution (aq.) (30 mL) and the mixture was extracted with dichloromethane (3×20 mL). The combined organic layers were washed with brine (3×30 mL), dried over anhydrous Na₂SO₄, and concentrated under reduced pressure. The residue was purified by silica gel column eluted with petroleum ether/ethyl acetate (1/3) to give 200 mg of 4-iodo-2-(2,2,2-trifluoroethyl)-1-{[2-(trimethylsilyl)ethoxy]methyl}pyrazol-3-one (INT-A20-5) as a brown oil (56%). ¹H NMR (DMSO, 400 MHz) δ −0.05 (9H, s), 0.74-0.87 (2H, m), 3.42-3.50 (2H, m), 4.72 (2H, q), 5.10 (2H, s), 8.35 (1H, s).

Step 6. Preparation of methyl 1-methyl-6-oxo-5-[3-oxo-2-(2,2,2-trifluoroethyl)-1-{[2-(trimethylsilyl)ethoxy]methyl}pyrazol-4-yl]pyridine-3-carboxylate (INT-A20-6). A mixture of methyl 5-bromo-1-methyl-6-oxopyridine-3-carboxylate (561 mg, 2.28 mmol mmol, 1 equiv), Pd(dppf)Cl₂·CH₂Cl₂ (167 mg, 0.23 mmol, 0.1 equiv), bis(pinacolato)diboron (868 mg, 3.42 mmol, 1.5 equiv), and KOAc (671 mg, 6.84 mmol, 3 equiv) in 1,4-dioxane (6 mL) was stirred overnight at 80 °C under nitrogen atmosphere. The mixture was cooled to room temperature and followed by the addition of 4-iodo-2-(2,2,2-trifluoroethyl)-1-{[2-(trimethylsilyl)ethoxy]methyl}pyrazol-3-one (966 mg, 2.28 mmol, 1 equiv), K₂CO₃ (948 mg, 6.86 mmol, 3 equiv), Pd(dppf)Cl₂ (167 mg, 0.23 mmol, 0.1 equiv), 1,4-dioxane (10 mL), and H₂O (5 mL). The reaction mixture was stirred for 2 h at 80 °C under nitrogen atmosphere. The mixture was cooled to room temperature. The reaction was quenched with water (50 mL) and the mixture was extracted with dichloromethane (3×50 mL). The combined organic layers were washed with brine (3×50 mL), dried over anhydrous Na₂SO₄, and concentrated under reduced pressure. The residue was purified by Prep-TLC eluted with petroleum ether/ethyl acetate (1/5) to give 1 g of methyl 1-methyl-6-oxo-5-[3-oxo-2-(2,2,2-trifluoroethyl)-1-{[2-(trimethylsilyl)ethoxy]methyl}pyrazol-4-yl]pyridine-3-carboxylate (INT-A20-6) as a brown solid (94%). LCMS: m/z (ESI), [M+H]⁺=462.00.

Step 7. Preparation of methyl 5-[5-hydroxy-1-(2,2,2-trifluoroethyl)pyrazol-4-yl]-1-methyl-6-oxopyridine-3-carboxylate (INT-A20-7). A mixture of methyl 1-methyl-6-oxo-5-[3-oxo-2-(2,2,2-trifluoroethyl)-1-{[2-(trimethylsilyl)ethoxy]methyl}pyrazol-4-yl]pyridine-3-carboxylate (1 g, 2.17 mmol, 1 equiv) and HCl in 1,4-dioxane (20 mL) was stirred for 2 h at room temperature under nitrogen atmosphere. The resulting mixture was concentrated under reduced pressure. The residue was treated with saturated NaHCO₃ (aq.) solution (100 mL) and extracted with dichloromethane (3×100 mL). The combined organic layers were washed with brine (3×200 mL), dried over anhydrous Na₂SO₄, and concentrated under reduced pressure to give 700 mg of methyl 5-[5-hydroxy-1-(2,2,2-trifluoroethyl)pyrazol-4-yl]-1-methyl-6-oxopyridine-3-carboxylate (INT-A20-7) as a brown solid (97%). ¹H NMR (DMSO, 400 MHz) δ 3.67 (3H, s), 3.84 (3H, s), 4.78 (2H, q), 8.11 (1H, s), 8.33 (1H, s), 8.50 (1H, d).

Step 8. Preparation of tert-butyl 4-(3-{[(2R)-5-({4-[5-(methoxycarbonyl)-1-methyl-2-oxopyridin-3-yl]-2-(2,2,2-trifluoroethyl)pyrazol-3-yl}oxy)-2-methylpentyl]amino}-4-nitrophenyl)piperazine-1-carboxylate (INT-A20-8). To a stirred solution of methyl 5-[5-hydroxy-1-(2,2,2-trifluoroethyl)pyrazol-4-yl]-1-methyl-6-oxopyridine-3-carboxylate (970 mg, 2.93 mmol, 1.00 equiv) and triphenylphosphine (2.304 g, 8.78 mmol, 3 equiv) in tetrahydrofuran (15 mL) were added tert-butyl 4-(3-{[(2R)-5-hydroxy-2-methylpentyl]amino}-4-nitrophenyl)piperazine-1-carboxylate (INT-A20-2, 1.237 g, 2.93 mmol, 1.00 equiv) and diisopropyl azodicarboxylate (1.776 g, 8.78 mmol, 3 equiv) at 0 °C under nitrogen atmosphere. The resulting mixture was stirred for 2 h at 0 °C under nitrogen atmosphere. The reaction was quenched with water (100 mL) and the mixture was extracted with ethyl acetate (3×100 mL). The combined organic layers were washed with brine (3×100 mL), dried over anhydrous Na₂SO₄, and concentrated under reduced pressure. The residue was purified by Prep-TLC eluted with petroleum ether/ethyl acetate to afford 1.2 g of tert-butyl 4-(3-{[(2R)-5-({4-[5-(methoxycarbonyl)-1-methyl-2-oxopyridin-3-yl]-2-(2,2,2-trifluoroethyl)pyrazol-3-yl}oxy)-2-methylpentyl]amino}-4-nitrophenyl)piperazine-1-carboxylate (INT-A20-8) as a brown oil (55%). LCMS: m/z (ESI), [M+H]⁺=736.25.

Step 9. Preparation of tert-butyl 4-(4-amino-3-{[(2R)-5-({4-[5-(methoxycarbonyl)-1-methyl-2-oxopyridin-3-yl]-2-(2,2,2-trifluoroethyl)pyrazol-3-yl}oxy)-2-methylpentyl]amino}phenyl)piperazine-1-carboxylate (INT-A20-9). To a stirred mixture of tert-butyl 4-(3-{[(2R)-5-({4-[5-(methoxycarbonyl)-1-methyl-2-oxopyridin-3-yl]-2-(2,2,2-trifluoroethyl)pyrazol-3-yl}oxy)-2-methylpentyl]amino}-4-nitrophenyl)piperazine-1-carboxylate (600 mg, 0.81 mmol, 1 equiv) and Raney-Ni (699 mg, 8.15 mmol, 10 equiv) in methanol (5 mL) was added hydrazine hydrate (61 mg, 1.22 mmol, 1.5 equiv) at 0 °C under nitrogen atmosphere. The resulting mixture was stirred for 2 h at room temperature. The resulting mixture was filtered. The filter cake was washed with methanol (3×30 mL) and the solution was concentrated under reduced pressure to give 575 mg of 4-(4-amino-3-{[(2R)-5-({4-[5-(methoxycarbonyl)-1-methyl-2-oxopyridin-3-yl]-2-(2,2,2-trifluoroethyl)pyrazol-3-yl}oxy)-2-methylpentyl]amino}phenyl)piperazine-1-carboxylate (INT-A20-9) as yellow. This material was used for the next reaction without further purification. LCMS: m/z (ESI), [M+H]⁺=706.45.

Step 10. Preparation of tert-butyl 4-{2-amino-3-[(2R)-5-({4-[5-(methoxycarbonyl)-1-methyl-2-oxopyridin-3-yl]-2-(2,2,2-trifluoroethyl)pyrazol-3-yl}oxy)-2-methylpentyl]-1,3-benzodiazol-5-yl}piperazine-1-carboxylate (INT-A20-10). A solution of tert-butyl 4-(4-amino-3-{[(2R)-5-({4-[5-(methoxycarbonyl)-1-methyl-2-oxopyridin-3-yl]-2-(2,2,2-trifluoroethyl)pyrazol-3-yl}oxy)-2-methylpentyl]amino}phenyl)piperazine-1-carboxylate (600 mg, 0.85 mmol, 1 equiv) and BrCN (108 mg, 1.02 mmol, 1.2 equiv) in dichloromethane (8 mL) was stirred for overnight at room temperature under nitrogen atmosphere. The resulting mixture was concentrated under vacuum. The residue was purified by Prep-TLC eluted with dichloromethane/methanol (10/1) to give of 180 mg of tert-butyl 4-{2-amino-3-[(2R)-5-({4-[5-(methoxycarbonyl)-1-methyl-2-oxopyridin-3-yl]-2-(2,2,2-trifluoroethyl)pyrazol-3-yl}oxy)-2-methylpentyl]-1,3-benzodiazol-5-yl}piperazine-1-carboxylate (INT-A20-10) as a purple solid (29%). ¹H NMR (DMSO, 400 MHz) δ 0.81 (4H, d), 1.15-1.34 (4H, m), 1.36-1.48 (11H, m), 1.70-1.76 (1H, m), 1.85-1.91 (1H, m), 1.97-2.05 (1H, m), 2.93-2.99 (4H, m), 3.30-3.37 (3H, m), 3.55-3.63 (3H, m), 3.75-3.83 (3H, m), 3.82-3.98 (5H, m), 4.86-4.96 (2H, m), 5.77 (6H, s), 6.67-6.77 (1H, m), 6.90 (1H, s), 6.97-7.16 (3H, m), 8.03 (1H, s), 8.05 (1H, d), 8.52 (1H, d).

Step 11. Preparation of 5-(5-{[(4R)-4-({2-amino-6-[4-(tert-butoxycarbonyl)piperazin-1-yl]-1,3-benzodiazol-1-yl}methyl)pentyl]oxy}-1-(2,2,2-trifluoroethyl)pyrazol-4-yl)-1-methyl-6-oxopyridine-3-carboxylic acid (INT-A20-11). A mixture of tert-butyl 4-{2-amino-3-[(2R)-5-({4-[5-(methoxycarbonyl)-1-methyl-2-oxopyridin-3-yl]-2-(2,2,2-trifluoroethyl)pyrazol-3-yl}oxy)-2-methylpentyl]-1,3-benzodiazol-5-yl}piperazine-1-carboxylate (150 mg, 0.20 mmol, 1 equiv) and LiOH·H₂O (13 mg, 0.31 mmol, 1.5 equiv) in tetrahydrofuran (4 mL) and H₂O (1 mL) was stirred for 2 h at room temperature under nitrogen atmosphere. The resulting mixture was concentrated under reduced pressure. The residue was purified by reverse flash chromatography on C18 silica gel column eluted with acetonitrile in water (0% to 100% gradient) to afford 70 mg of 5-(5-{[(4R)-4-({2-amino-6-[4-(tert-butoxycarbonyl)piperazin-1-yl]-1,3-benzodiazol-1-yl}methyl)pentyl]oxy}-1-(2,2,2-trifluoroethyl)pyrazol-4-yl)-1-methyl-6-oxopyridine-3-carboxylic acid (INT-A20-11) as a purple solid (47%). ¹H NMR (DMSO, 400 MHz) δ 0.76 (3H, d), 1.25 (1H, s), 1.42 (9H, s), 1.54-1.61 (1H, m), 1.70-1.76 (1H, m), 1.85-1.91 (1H, m), 1.97-2.05 (1H, s), 2.93-2.99 (4H, m), 3.30-3.37 (3H, m), 3.55-3.63 (3H, m), 3.75-3.83 (3H, m), 3.82-3.98 (5H, m), 4.88 (2H, m), 6.54 (2H, s), 6.60 (1H, q), 6.79 (1H, d), 6.96 (1H, d), 8.02 (1H, s), 8.04 (1H, d), 8.25 (1H, d).

Step 12. Preparation of tert-butyl 4-[(11R)-11,26-dimethyl-23,27-dioxo-5-(2,2,2-trifluoroethyl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaen-16-yl]piperazine-1-carboxylate (INT-A20-12). A mixture of N,N-diisopropylethylamine (27 mg, 0.21 mmol, 3 equiv), 5-(5-{[(4R)-4-({2-amino-6-[4-(tert-butoxycarbonyl)piperazin-1-yl]-1,3-benzodiazol-1-yl}methyl)pentyl]oxy}-1-(2,2,2-trifluoroethyl)pyrazol-4-yl)-1-methyl-6-oxopyridine-3-carboxylic acid (50 mg, 0.07 mmol, 1 equiv), and N,N,N,N-tetramethyl-O-(7-azabenzotriazol-1-yl)uronium hexafluorophospate (40 mg, 0.10 mmol, 1.5 equiv) in 1,4-dioxane (2 mL) was stirred overnight at 60 °C under nitrogen atmosphere. The mixture was cooled to room temperature. The reaction was quenched with water (30 mL) and the mixture was extracted with ethyl acetate (3×20 mL). The combined organic layers were washed with brine (3×20 mL), dried over anhydrous Na₂SO₄, and concentrated under reduced pressure. The residue was purified by Prep-TLC eluted with dichloromethane/methanol (10/1) to give 48 mg of tert-butyl 4-[(11R)-11,26-dimethyl-23,27-dioxo-5-(2,2,2-trifluoroethyl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaen-16-yl]piperazine-1-carboxylate (INT-A20-12) as a yellow solid (98%). LCMS: m/z (ESI), [M+H]⁺=699.45.

Step 13. Preparation of (11R)-11,26-dimethyl-16-(piperazin-1-yl)-5-(2,2,2-trifluoroethyl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (INT-A20-13). A mixture of tert-butyl 4-[(11R)-11,26-dimethyl-23,27-dioxo-5-(2,2,2-trifluoroethyl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaen-16-yl]piperazine-1-carboxylate (INT-A20-13 40 mg, 0.06 mmol, 1 equiv) and trifluoroacetic acid (0.5 mL) in dichloromethane (2 mL) was stirred for 1 h at room temperature under nitrogen atmosphere. The resulting mixture was concentrated under reduced pressure. The residue was treated with saturated NaHCO₃ (aq.) solution (100 mL) and extracted with dichloromethane (3×100 mL). The combined organic layers were washed with brine (3×200 mL), dried over anhydrous Na₂SO₄, and concentrated under reduced pressure. The crude product was used in the next step directly without further purification. LCMS: m/z (ESI), [M+H]⁺=599.15.

Step 14. Preparation of (11R)-11,26-dimethyl-16-(4-methylpiperazin-1-yl)-5-(2,2,2-trifluoroethyl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (EXAMPLE A20). To a stirred mixture of (11R)-11,26-dimethyl-16-(piperazin-1-yl)-5-(2,2,2-trifluoroethyl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (40 mg, 0.07 mmol, 1 equiv), trimethylamine (20 mg, 0.20 mmol, 3 equiv) and paraformaldehyde (6 mg, 0.06 mmol, 1 equiv) in dichloromethane (2 mL) was added sodium triacetoxyborohydride (42 mg, 0.20 mmol, 3 equiv) at room temperature. The resulting mixture was stirred for 2 h at room temperature under nitrogen atmosphere and concentrated under reduced pressure. The residue was purified by Prep-TLC eluted with dichloromethane/methanol (5/1) to give 6.7 mg of (11R)-11,26-dimethyl-16-(4-methylpiperazin-1-yl)-5-(2,2,2-trifluoroethyl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (EXAMPLE A20) as a yellow green solid (16%). LCMS: m/z (ESI), [M+H]⁺=613.30. ¹H NMR (DMSO-d₆, 400 MHz) δ 0.77-0.89 (5H, m), 1.11-1.29 (9H, m), 1.40-1.50 (2H, m), 1.85-1.92 (2H, m), 2.15-2.21 (2H, m), 2.28 (3H, s), 2.75-2.80 (1H, m), 3.14-3.20 (3H, m), 3.63 (3H, s), 3.90-3.96 (2H, m), 4.07-4.11 (1H, m), 4.43-4.48 (1H, m), 4.93-5.00 (1H, m), 6.87 (1H, d), 7.14 (1H, s), 7.36 (1H, d), 8.31 (1H, d), 8.46 (1H, s), 8.73 (1H, d), 12.37 (1H, s)

### Example A21

### (11R)-5-ethyl-11,26-dimethyl-16-(4-methylpiperazin-1-yl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione

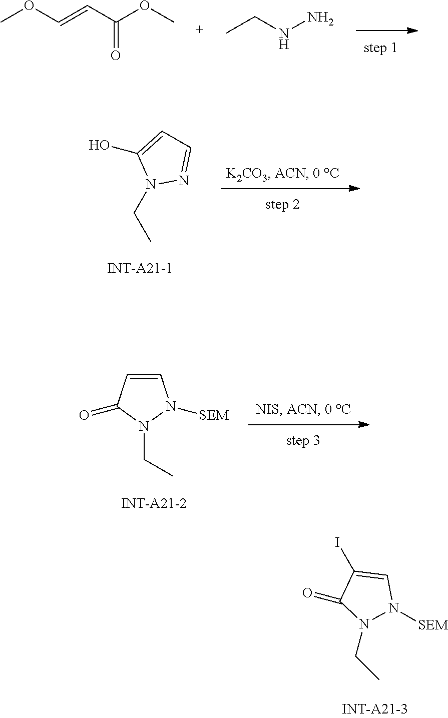

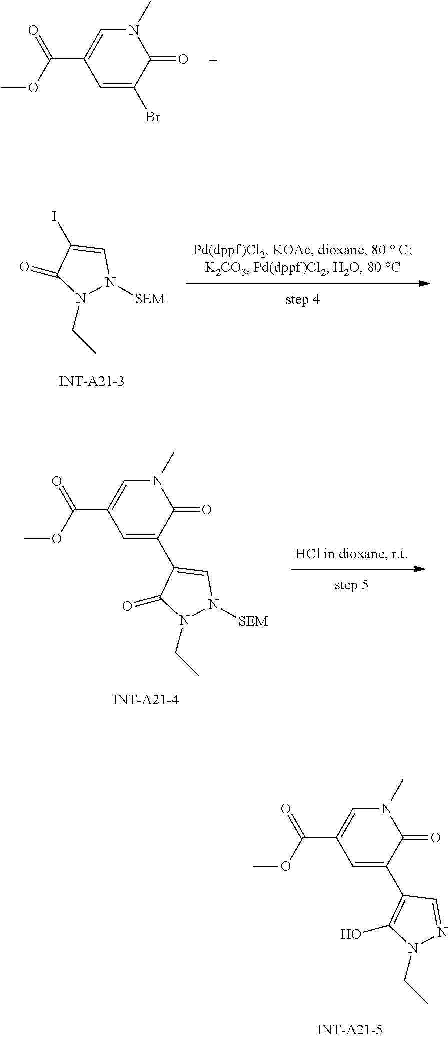 [SMILES: COC(=O)c1cc(Br)c(=O)n(C)c1 ; CCn1c(=O)c(I)cn1COCC[Si](C)(C)C ; CCn1c(=O)c(-c2cc(C(=O)OC)cn(C)c2=O)cn1COCC[Si](C)(C)C ; CCn1ncc(-c2cc(C(=O)OC)cn(C)c2=O)c1O]

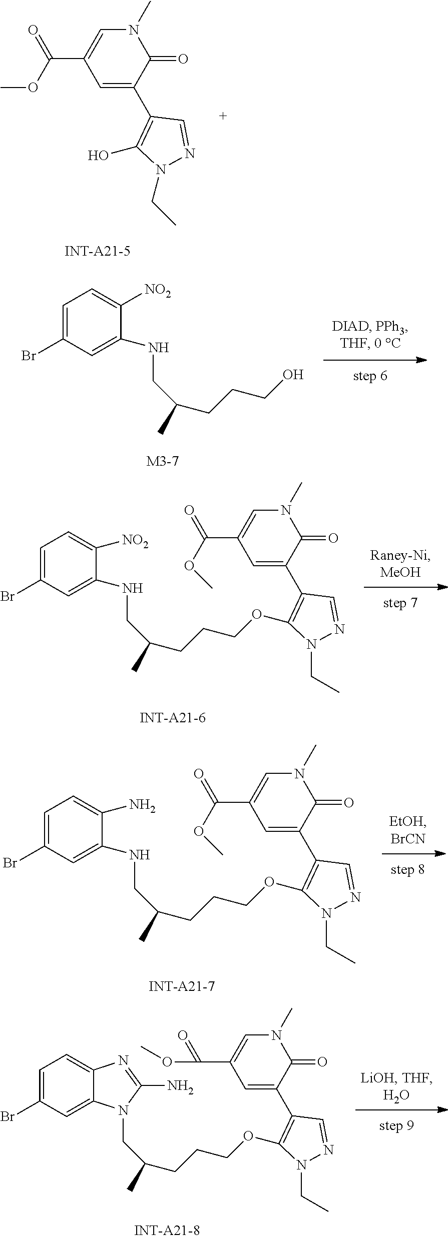 [SMILES: CCn1ncc(-c2cc(C(=O)OC)cn(C)c2=O)c1O ; C[C@H](CCCO)CNc1cc(Br)ccc1[N+](=O)[O-] ; CCn1ncc(-c2cc(C(=O)OC)cn(C)c2=O)c1OCCC[C@@H](C)CNc1cc(Br)ccc1[N+](=O)[O-] ; CCn1ncc(-c2cc(C(=O)OC)cn(C)c2=O)c1OCCC[C@@H](C)CNc1cc(Br)ccc1N ; CCn1ncc(-c2cc(C(=O)OC)cn(C)c2=O)c1OCCC[C@@H](C)Cn1c(N)nc2ccc(Br)cc21]

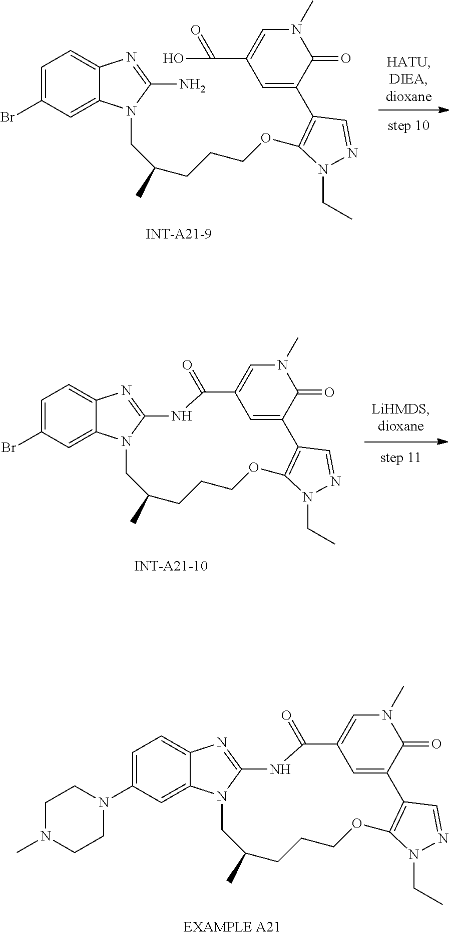 [SMILES: CCn1ncc(-c2cc(C(=O)O)cn(C)c2=O)c1OCCC[C@@H](C)Cn1c(N)nc2ccc(Br)cc21 ; CCn1ncc2c1OCCC[C@@H](C)Cn1c(nc3ccc(Br)cc31)NC(=O)c1cc-2c(=O)n(C)c1 ; CCn1ncc2c1OCCC[C@@H](C)Cn1c(nc3ccc(N4CCN(C)CC4)cc31)NC(=O)c1cc-2c(=O)n(C)c1]

Step 1. Preparation of 2-ethylpyrazol-3-ol (INT-A21-1). A mixture of ethylhydrazine dihydrochloride (5.7 g, 43.06 mmol, 1 equiv) and methyl (2E)-3-methoxyprop-2-enoate (5 g, 43.06 mmol, 1 equiv) in methanol (50 mL) was stirred overnight at 80 °C under nitrogen atmosphere. After being cooled to room temperature, the reaction mixture was concentrated under reduced pressure and the residue was purified by silica gel chromatography eluted with petroleum ether/ethyl acetate (1/1) to give 4 g of 2-ethylpyrazol-3-ol (INT-A21-1) as a white solid (820%). H NMR (DMSO-d, 400 MHz) δ 1.26 (3H, t), 3.94-3.98 (2H, m), 5.82 (1H, m), 7.48 (1H, d), 10.99 (1H, s).

Step 2. Preparation of 2-ethyl-1-{[2-(trimethylsilyl)ethoxy]methyl}pyrazol-3-one (INT-A21-2). A mixture of 2-ethylpyrazol-3-ol (5 g, 44.59 mmol, 1 equiv), 2-(trimethylsily)ethoxymethyl chloride (11.15 g, 66.88 mmol, 1.5 equiv), and K₂CO₃ (15.41 g, 111.47 mmol, 2.5 equiv) in acetonitrile (50 mL) was stirred for 0.5 h at room temperature under nitrogen atmosphere. The reaction was quenched with water (500 mL) and the mixture was extracted with ethyl acetate (3×500 mL). The combined organic layers were washed with brine (3×500 mL), dried over anhydrous Na₂SO₄, and concentrated under reduced pressure. The residue was purified by silica gel chromatography eluted with petroleum ether/ethyl acetate (1/1) to give 1.5 g of 2-ethyl-1-{[2-(trimethylsilyl)ethoxy]methyl}pyrazol-3-one (INT-A21-2) as a yellow oil (13%). ¹H NMR (DMSO-d₆, 400 MHz) δ 0.00 (9H, s), 0.79-0.84 (2H, m), 1.04 (3H, t), 3.38-3.47 (2H, m), 3.77 (2H, q), 5.08 (2H, s), 5.27 (1H, d), 7.89 (1H, d)

Step 3. Preparation of 2-ethyl-4-iodo-1-{[2-(trimethylsilyl)ethoxy]methyl}pyrazol-3-one (INT-A21-3). A mixture of 2-ethyl-1-{[2-(trimethylsilyl)ethoxy]methyl}pyrazol-3-one (1.15 g, 4.74 mmol, 1 equiv) and N-iodosuccinimide (1.28 g, 5.69 mmol, 1.2 equiv) in acetonitrile (15 mL) was stirred for 3 h at 0 °C under nitrogen atmosphere. The reaction was quenched with water (100 mL) and the mixture was extracted with ethyl acetate (3×100 mL). The combined organic layers were washed with brine (3×100 mL), dried over anhydrous Na₂SO₄, and concentrated under reduced pressure. The residue was purified by silica gel chromatography eluted with petroleum ether/ethyl acetate (1/1) to give 1.9 g of 2-ethyl-4-iodo-1-{[2-(trimethylsilyl)ethoxy]methyl}pyrazol-3-one (INT-A21-3) as a yellow solid (97%). ¹H NMR (DMSO-d₆, 400 MHz) δ 0.03 (9H, s), 0.81 (2H, t), 1.05 (3H, t), 3.43 (2H, t), 3.83 (2H, q), 5.09 (2H, s), 8.17 (1H, s).

Step 4. Preparation of methyl 5-(2-ethyl-3-oxo-1-{[2-(trimethylsilyl)ethoxy]methyl}pyrazol-4-yl)-1-methyl-6-oxopyridine-3-carboxylate (INT-A21-4). A mixture of methyl 5-bromo-1-methyl-6-oxopyridine-3-carboxylate (1.4 g, 5.69 mmol, 1 equiv), bis(pinacolato)diboron (1.73 g, 6.82 mmol, 1.2 equiv), KOAc (1.4 g, 14.22 mmol, 2.5 equiv) and Pd(dppf)Cl₂ dichloromethane (0.46 g, 0.57 mmol, 0.1 equiv) in dioxane (16 mL) was stirred for 2 h at 80 °C under nitrogen atmosphere. The reaction mixture was cooled to room temperature prior to the addition of 2-ethyl-4-iodo-1-{[2-(trimethylsilyl)ethoxy]methyl}pyrazol-3-one (1.7 g, 4.61 mmol, 0.9 equiv), Pd(dppf)Cl₂·dichloromethane (0.28 g, 0.51 mmol, 0.1 equiv), K₂CO₃ (1.91 g, 13.82 mmol, 2.7 equiv), and H₂O (4 mL). The reaction was stirred for additional 2 h at 80 °C under nitrogen atmosphere. After being cooled to room temperature, the reaction was quenched with water (100 mL) and the mixture was extracted with ethyl acetate (3×100 mL). The combined organic layers were washed with brine (3×100 mL), dried over anhydrous Na₂SO₄, and concentrated under reduced pressure. The residue was purified by silica gel chromatography eluted with dichloromethane/methanol (10/1) to give to give 1.6 g of methyl 5-(2-ethyl-3-oxo-1-{[2-(trimethylsilyl)ethoxy]methyl}pyrazol-4-yl)-1-methyl-6-oxopyridine-3-carboxylate (INT-A21-4) as a yellow solid (76%). LCMS: m/z (ESI), [M+H]⁺=408.10. ¹H NMR (DMSO-d₆, 400 MHz) δ 0.05 (9H, s), 0.82 (2H, t), 1.10-1.13 (3H, m), 3.47 (2H, t), 3.60 (3H, s), 3.81 (3H, s), 3.89 (2H, q), 5.29 (2H, s), 8.43 (1H, d), 8.88 (1H, s), 9.21 (1H, d).

Step 5. Preparation of methyl 5-(1-ethyl-5-hydroxypyrazol-4-yl)-1-methyl-6-oxopyridine-3-carboxylate (INT-A21-5). A solution of methyl 5-(2-ethyl-3-oxo-1-{[2-(trimethylsilyl)ethoxy]methyl}pyrazol-4-yl)-1-methyl-6-oxopyridine-3-carboxylate (1.6 g, 3.92 mmol, 1 equiv) and HCl (20 mL, 4M in 1,4-dioxane) was stirred for 2 h at room temperature. The residue was treated with dichloromethane (30 mL) and treated with K₂CO₃. The mixture was filtered. The filter cake was washed with dichloromethane (3×20 mL). The resulting solution was concentrated under reduced pressure to give 640 mg of methyl 5-(1-ethyl-5-hydroxypyrazol-4-yl)-1-methyl-6-oxopyridine-3-carboxylate (INT-A21-5) as a yellow solid. This material was used for the next reaction without further purification. LCMS: m/z (ESI), [M+H]⁺=278.00.

Step 6. Preparation of methyl 5-(5-{[(4R)-5-[(5-bromo-2-nitrophenyl)amino]-4-methylpentyl]oxy}-1-ethylpyrazol-4-yl)-1-methyl-6-oxopyridine-3-carboxylate (INT-A21-6). A mixture of methyl 5-(1-ethyl-5-hydroxypyrazol-4-yl)-1-methyl-6-oxopyridine-3-carboxylate (670 mg, 2.41 mmol, 1 equiv) and triphenylphosphine (1.58 g, 6.04 mmol, 2.5 equiv) in tetrahydrofuran (10 mL) was treated with diisopropylazodicarboxylate (0.98 g, 4.83 mmol, 2 equiv) for 1 h at 0 °C under nitrogen atmosphere followed by the addition of (4R)-5-[(5-bromo-2-nitrophenyl)amino]-4-methylpentan-1-ol (M3-7, 766 mg, 2.41 mmol, 1 equiv) at 0 °C. After being stirred for additional 4 h at 0 °C under nitrogen atmosphere, the reaction was quenched with water (100 mL) and the mixture was extracted with ethyl acetate (3×100 mL). The combined organic layers were washed with brine (3×100 mL), dried over anhydrous Na₂SO₄, and concentrated under reduced pressure. The residue was purified by Prep-TLC eluted with dichloromethane/methanol (10/1) to give 720 mg of methyl 5-(5-{[(4R)-5-[(5-bromo-2-nitrophenyl)amino]-4-methylpentyl]oxy}-1-ethylpyrazol-4-yl)-1-methyl-6-oxopyridine-3-carboxylate (INT-A21-6) as a yellow solid (51%). LCMS: m/z (ESI), [M+H]⁺=578.00.

Step 7. Preparation of methyl 5-(5-{[(4R)-5-[(2-amino-5-bromophenyl)amino]-4-methylpentyl]oxy}-1-ethylpyrazol-4-yl)-1-methyl-6-oxopyridine-3-carboxylate (INT-A21-7). A mixture of methyl 5-(5-{[(4R)-5-[(5-bromo-2-nitrophenyl)amino]-4-methylpentyl]oxy}-1-ethylpyrazol-4-yl)-1-methyl-6-oxopyridine-3-carboxylate (720 mg, 1.25 mmol, 1 equiv), Raney-Ni (54 mg, 0.62 mmol, 0.5 equiv) and hydrazine (120 mg, 3.74 mmol, 3 equiv) in methanol (15 mL) was stirred for 2 h at room temperature. The resulting mixture was filtered and the filter cake was washed with methanol (3×20 mL). The resulting solution was concentrated under reduced pressure to give 600 mg of methyl 5-(5-{[(4R)-5-[(2-amino-5-bromophenyl)amino]-4-methylpentyl]oxy}-1-ethylpyrazol-4-yl)-1-methyl-6-oxopyridine-3-carboxylate (INT-A21-7) as a yellow oil. This material was used for the next reaction without further purification. LCMS: m/z (ESI), [M+H]⁺=546.20.

Step 8. Preparation of methyl 5-(5-{[(4R)-4-[(2-amino-6-bromo-1,3-benzodiazol-1-yl)methyl]pentyl]oxy}-1-ethylpyrazol-4-yl)-1-methyl-6-oxopyridine-3-carboxylate (INT-A21-8). A mixture of methyl 5-(5-{[(4R)-5-[(2-amino-5-bromophenyl)amino]-4-methylpentyl]oxy}-1-ethylpyrazol-4-yl)-1-methyl-6-oxopyridine-3-carboxylate (600 mg, 1.10 mmol, 1 equiv) and cyanogen bromide (232 mg, 2.20 mmol, 2 equiv) in ethanol (15 mL) was stirred for 2 h at room temperature and concentrated. The residue was purified by silica gel chromatography eluted with dichloromethane/methanol (10/1) to give 400 mg of methyl 5-(5-{[(4R)-4-[(2-amino-6-bromo-1,3-benzodiazol-1-yl)methyl]pentyl]oxy}-1-ethylpyrazol-4-yl)-1-methyl-6-oxopyridine-3-carboxylate (INT-A21-8) as a yellow oil (63%). LCMS: m/z (EST), [M+H]⁺=573.30.

Step 9. Preparation of 5-(5-{[(4R)-4-[(2-amino-6-bromo-1,3-benzodiazol-1-yl)methyl]pentyl]oxy}-1-ethylpyrazol-4-yl)-1-methyl-6-oxopyridine-3-carboxylic acid (INT-A21-9). To a stirred mixture of methyl 5-(5-{[(4R)-4-[(2-amino-6-bromo-1,3-benzodiazol-1-yl)methyl]pentyl]oxy}-1-ethylpyrazol-4-yl)-1-methyl-6-oxopyridine-3-carboxylate (700 mg, 1.22 mmol, 1 equiv) in tetrahydrofuran (15 mL) was added LiOH (58 mg, 2.45 mmol, 2 equiv) in H₂O (3 mL). The resulting mixture was stirred for 2 h at room temperature and concentrated under reduced pressure. The residue was purified by reverse flash chromatography eluted with 10% to 50% acetonitrile in water to give 600 mg of 5-(5-{[(4R)-4-[(2-amino-6-bromo-1,3-benzodiazol-1-yl)methyl]pentyl]oxy}-1-ethylpyrazol-4-yl)-1-methyl-6-oxopyridine-3-carboxylic acid (INT-A21-9) as a white solid (87%). LCMS: m/z (ESI), [M+H]⁺=559.30.

Step 10. Preparation of (11R)-16-bromo-5-ethyl-11,26-dimethyl-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (INT-A21-10). A mixture of 5-(5-{[(4R)-4-[(2-amino-6-bromo-1,3-benzodiazol-1-yl)methyl]pentyl]oxy}-1-ethylpyrazol-4-yl)-1-methyl-6-oxopyridine-3-carboxylic acid (460 mg, 0.82 mmol, 1 equiv), N,N,N,N-tetramethyl-O-(7-azabenzotriazol-1-yl)uronium hexafluorophospate (627 mg, 1.65 mmol, 2 equiv) and N,N-diisopropylethyl amine (213 mg, 1.65 mmol, 2 equiv) in dioxane (10 mL) was stirred for 2 h at room temperature under nitrogen atmosphere. The reaction was quenched with water (100 mL) and the mixture was extracted with ethyl acetate (3×100 mL). The combined organic layers were washed with brine (3×100 mL), dried over anhydrous Na₂SO₄, and concentrated under reduced pressure. The residue was purified by Prep-TLC eluted with dichloromethane/methanol (15/1) to give 430 mg of (11R)-16-bromo-5-ethyl-11,26-dimethyl-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (INT-A21-10) as a white solid (96%). LCMS: m/z (EST), [M+H]⁺=541.00.

Step 11. Preparation of (11R)-5-ethyl-11,26-dimethyl-16-(4-methylpiperazin-1-yl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (EXAMPLE. A21). To a mixture of (11R)-16-bromo-5-ethyl-11,26-dimethyl-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (100 mg, 0.18 mmol, 1 equiv), BrettPhos Pd G₃ (84.02 mg, 0.09 mmol, 0.5 equiv) and 1-methylpiperazine (74 mg, 0.74 mmol, 4 equiv) in 1,4-dioxane (5 mL) was added LiHMDS (1.1 mL, 1.11 mmol, 6 equiv) dropwise at room temperature under nitrogen atmosphere. The reaction mixture was stirred at 60 °C for 30 mins. After being cooled to room temperature, the reaction was quenched with sat. NH₄Cl solution (100 mL) and the mixture was extracted with ethyl acetate (3×100 mL). The combined organic layers were washed with brine (3×100 mL), dried over anhydrous Na₂SO₄, and concentrated under reduced pressure. The residue was purified by Prep-TLC eluted with dichloromethane/methanol (18/1) to give 29 mg of (11R)-5-ethyl-11,26-dimethyl-16-(4-methylpiperazin-1-yl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (EXAMPLE. A21) as a white solid (28%). LCMS: m/z (ESI), [M+H]⁺=559.35. ¹H NMR (DMSO-d₆, 400 MHz) δ 0.80 (3H, d), 1.37 (3H, t), 1.45 (1H, t), 1.90 (2H, d), 2.17-2.25 (4H, m), 2.51 (4H, t), 2.79 (1H, s), 3.15 (4H, t), 3.61 (3H, s), 3.86-4.13 (5H, m), 4.36-4.40 (1H, m), 6.86 (1H, d), 7.12 (1H, d), 7.35 (1H, d), 8.26 (1H, d), 8.37 (1H, s), 8.78 (1H, d), 12.35 (1H, s).

### Example A22

### (11R)-5-Cyclopropyl-11,26-dimethyl-16-(4-methylpiperazin-1-yl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione

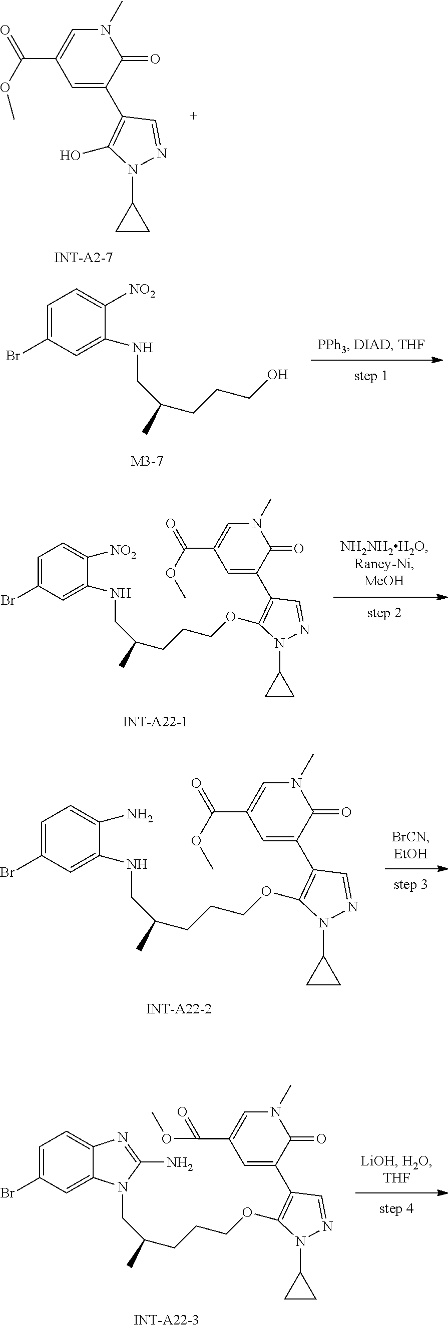 [SMILES: COC(=O)c1cc(-c2cnn(C3CC3)c2O)c(=O)n(C)c1 ; C[C@H](CCCO)CNc1cc(Br)ccc1[N+](=O)[O-] ; COC(=O)c1cc(-c2cnn(C3CC3)c2OCCC[C@@H](C)CNc2cc(Br)ccc2[N+](=O)[O-])c(=O)n(C)c1 ; COC(=O)c1cc(-c2cnn(C3CC3)c2OCCC[C@@H](C)CNc2cc(Br)ccc2N)c(=O)n(C)c1 ; COC(=O)c1cc(-c2cnn(C3CC3)c2OCCC[C@@H](C)Cn2c(N)nc3ccc(Br)cc32)c(=O)n(C)c1]

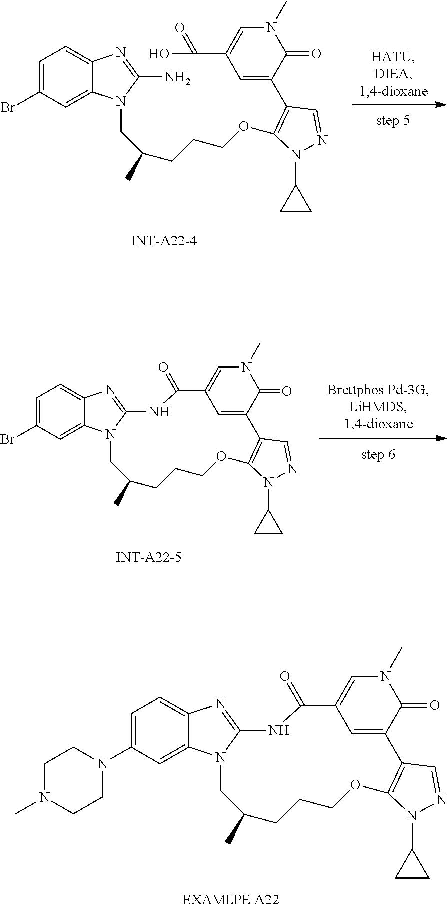 [SMILES: C[C@H](CCCOc1c(-c2cc(C(=O)O)cn(C)c2=O)cnn1C1CC1)Cn1c(N)nc2ccc(Br)cc21 ; C[C@@H]1CCCOc2c(cnn2C2CC2)-c2cc(cn(C)c2=O)C(=O)Nc2nc3ccc(Br)cc3n2C1 ; C[C@@H]1CCCOc2c(cnn2C2CC2)-c2cc(cn(C)c2=O)C(=O)Nc2nc3ccc(N4CCN(C)CC4)cc3n2C1]

Step 1. Preparation of methyl 5-(5-{[(4R)-5-[(5-bromo-2-nitrophenyl)amino]-4-methylpentyl]oxy}-1-cyclopropylpyrazol-4-yl)-1-methyl-6-oxopyridine-3-carboxylate (INT-A22-1). To a stirred mixture of methyl 5-(1-cyclopropyl-5-hydroxypyrazol-4-yl)-1-methyl-6-oxopyridine-3-carboxylate (INT-2-7, 750 mg, 2.59 mmol, 1 equiv) and triphenylphosphine (1360 mg, 5.19 mmol, 2 equiv) in tetrahydrofuran (10 mL) were added diisopropyl azodicarboxylate (1048 mg, 5.19 mmol, 2 equiv) and (4R)-5-[(5-bromo-2-nitrophenyl)amino]-4-methylpentan-1-ol (M3-7, 905 mg, 2.85 mmol, 1.1 equiv) at 0 °C. The resulting mixture was stirred for 2 h at 0 °C under nitrogen atmosphere. The reaction was quenched with water (30 mL) and the mixture was extracted with dichloromethane (3×30 mL). The combined organic layers were dried over anhydrous Na₂SO₄ and concentrated under reduced pressure. The residue was purified on a silica gel column eluted with petroleum ether/ethyl acetate (1/1) to give 700 mg of methyl 5-(5-{[(4R)-5-[(5-bromo-2-nitrophenyl)amino]-4-methylpentyl]oxy}-1-cyclopropylpyrazol-4-yl)-1-methyl-6-oxopyridine-3-carboxylate (INT-A22-1) as a yellow oil (45%). LCMS: m/z (EST), [M+H]+=590.10

Step 2. Preparation of methyl 5-(5-{[(4R)-5-[(2-amino-5-bromophenyl)amino]-4-methylpentyl]oxy}-1-cyclopropylpyrazol-4-yl)-1-methyl-6-oxopyridine-3-carboxylate (INT-A22-2). To a stirred mixture of methyl 5-(5-{[(4R)-5-[(5-bromo-2-nitrophenyl)amino]-4-methylpentyl]oxy}-1-cyclopropylpyrazol-4-yl)-1-methyl-6-oxopyridine-3-carboxylate (700 mg, 1.19 mmol, 1 equiv) and Raney Ni (51 mg, 0.60 mmol, 0.5 equiv) in methanol (15 mL) was added NH₂NH₂·H₂O (90 mg, 1.79 mmol, 1.5 equiv) at 0 °C. The mixture was stirred for 1 h at 0 °C under nitrogen atmosphere. The mixture was filtered. The filter cake was washed with methanol (2×20 mL). The resulting solution was concentrated under reduced pressure. The residue was purified on a silica gel column eluted with dichloromethane/methanol (10/1) to give 570 mg of methyl 5-(5-{[(4R)-5-[(2-amino-5-bromophenyl)amino]-4-methylpentyl]oxy}-1-cyclopropylpyrazol-4-yl)-1-methyl-6-oxopyridine-3-carboxylate (INT-A22-2) as a yellow solid (85%). LCMS: m/z (ESI), [M+H]⁺=560.40

Step 3. Preparation of methyl 5-(5-{[(4R)-4-[(2-amino-6-bromo-1,3-benzodiazol-1-yl)methyl]pentyl]oxy}-1-cyclopropylpyrazol-4-yl)-1-methyl-6-oxopyridine-3-carboxylate (INT-A22-3). A mixture of methyl 5-(5-{[(4R)-5-[(2-amino-5-bromophenyl)amino]-4-methylpentyl]oxy}-1-cyclopropylpyrazol-4-yl)-1-methyl-6-oxopyridine-3-carboxylate (570 mg, 1.02 mmol, 1 equiv) and BrCN (162 mg, 1.53 mmol, 1.5 equiv) in ethanol (5 mL) was stirred for 2 h at room temperature under nitrogen atmosphere. The mixture was concentrated under reduced pressure and the residue was purified on a silica gel column eluted with dichloromethane/methanol (10/1) to give 550 mg of methyl 5-(5-{[(4R)-4-[(2-amino-6-bromo-1,3-benzodiazol-1-yl)methyl]pentyl]oxy}-1-cyclopropylpyrazol-4-yl)-1-methyl-6-oxopyridine-3-carboxylate (INT-A22-3) as a brown solid (92%). LCMS: m/z (ESI), [M+H]⁺=585.15

Step 4. Preparation of 5-(5-{[(4R)-4-[(2-amino-6-bromo-1,3-benzodiazol-1-yl)methyl]pentyl]oxy}-1-cyclopropylpyrazol-4-yl)-1-methyl-6-oxopyridine-3-carboxylic acid (INT-A22-4). To a stirred mixture of methyl 5-(5-{[(4R)-4-[(2-amino-6-bromo-1,3-benzodiazol-1-yl)methyl]pentyl]oxy}-1-cyclopropylpyrazol-4-yl)-1-methyl-6-oxopyridine-3-carboxylate (480 mg, 0.823 mmol, 1 equiv) in tetrahydrofuran (20 mL) were added LiOH (78.81 mg, 3.292 mmol, 4 equiv) and H₂O (5 mL) at room temperature. The resulting mixture was stirred for 30 min at 60 °C. The mixture was cooled to room temperature and concentrated under reduced pressure. The residue was purified by reverse flash chromatography eluted with 10% to 40% acetonitrile in water to give 380 mg of 5-(5-{[(4R)-4-[(2-amino-6-bromo-1,3-benzodiazol-1-yl)methyl]pentyl]oxy}-1-cyclopropylpyrazol-4-yl)-1-methyl-6-oxopyridine-3-carboxylic acid (INT-A22-4) as an off-white solid (81%). LCMS: m/z (ESI), [M+H]⁺=571.00.

Step 5. Preparation of (11R)-16-bromo-5-cyclopropyl-11,26-dimethyl-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (INT-A22-5). A mixture of 5-(5-{[(4R)-4-[(2-amino-6-bromo-1,3-benzodiazol-1-yl)methyl]pentyl]oxy}-1-cyclopropylpyrazol-4-yl)-1-methyl-6-oxopyridine-3-carboxylic acid (375 mg, 0.66 mmol, 1 equiv), N,N-diisopropylethylamine (255 mg, 1.98 mmol, 3 equiv) and N,N,N,N-tetramethyl-O-(7-azabenzotriazol-1-yl)uronium hexafluorophospate (375 mg, 0.98 mmol, 1.5 equiv) in 1,4-dioxane (10 mL) was stirred for 2 h at 60 °C under nitrogen atmosphere. The reaction mixture was cooled to room temperature, treated with sat. NH₄Cl (aq.) (20 mL), and extracted with dichloromethane (3×20 mL). The combined organic layers were washed with brine (3×30 mL), dried over anhydrous Na₂SO₄, and concentrated under reduced pressure. The residue was purified on a silica gel column eluted with dichloromethane/methanol (10/1) to give 330 mg of (11R)-16-bromo-5-cyclopropyl-11,26-dimethyl-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (INT-A22-5) as a reddish solid (90%). LCMS: m/z (ESI), [M+H]⁺=553.10.

Step 6. Preparation of (11R)-5-cyclopropyl-11,26-dimethyl-16-(4-methylpiperazin-1-yl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (EXAMPLE A22). To a stirred mixture of (11R)-16-bromo-5-cyclopropyl-11,26-dimethyl-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (90 mg, 0.16 mmol, 1 equiv), BrettPhos Pd G₃ (44 mg, 0.05 mmol, 0.3 equiv), and 1-methylpiperazine (49 mg, 0.49 mmol, 3 equiv) in 1,4-dioxane (9 mL) was added LiHMDS (2.7 mL, 2.7 mmol, 16.9 equiv) at room temperature under nitrogen atmosphere. The mixture was stirred for 30 min at 60 °C under nitrogen atmosphere and cooled to room temperature. The reaction was quenched with sat. NH₄Cl (aq.) solution (30 mL) and the mixture was extracted with dichloromethane (3×20 mL). The combined organic layers were washed with brine (3×30 mL), dried over anhydrous Na₂SO₄, and concentrated under reduced pressure. The residue was purified on a silica gel column eluted with dichloromethane/methanol (15/1) to give 24.7 mg of the title compound (EXAMPLE A22) as an off-white solid (26%). LCMS: m/z (ESI), [M+H]⁺=571.40. ¹H NMR (DMSO-d₆, 400 MHz) δ 0.81 (3H, d),C 0.971.06 (4H, m), 1.37-1.54 (1H, m), 1.83-2.03 (2H, m), 2.17-2.23 (1H, m), 2.24 (3H, s), 2.50 (4H, t), 2.82 (1H, br s), 3.15 (4H, br s), 3.53-3.60 (1H, m), 3.61 (3H, s), 3.85-4.20 (3H, m), 4.30-4.47 (1H, m), 6.86 (1H, dd), 7.13 (1H, d), 7.35 (1H, d), 8.26 (1H, d), 8.30 (1H, s), 8.84 (1H, d), 12.35 (1H, s).

### Example A23

### (11R)-15-Chloro-5,11,26-trimethyl-16-(4-methylpiperazin-1-yl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione

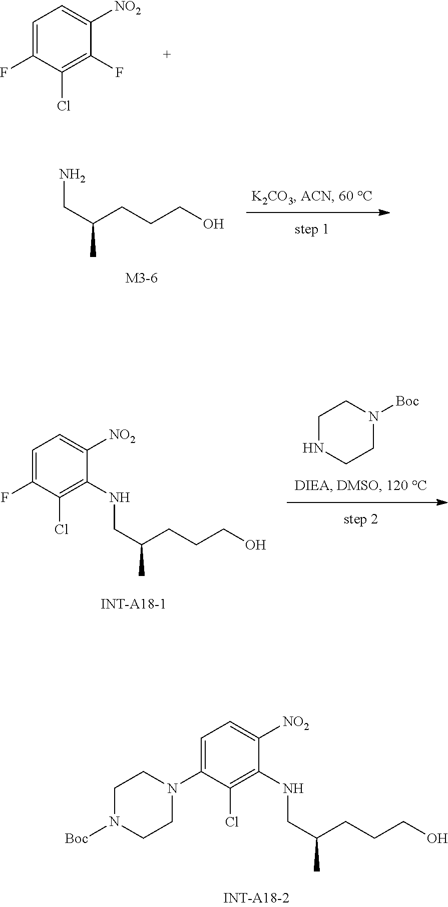

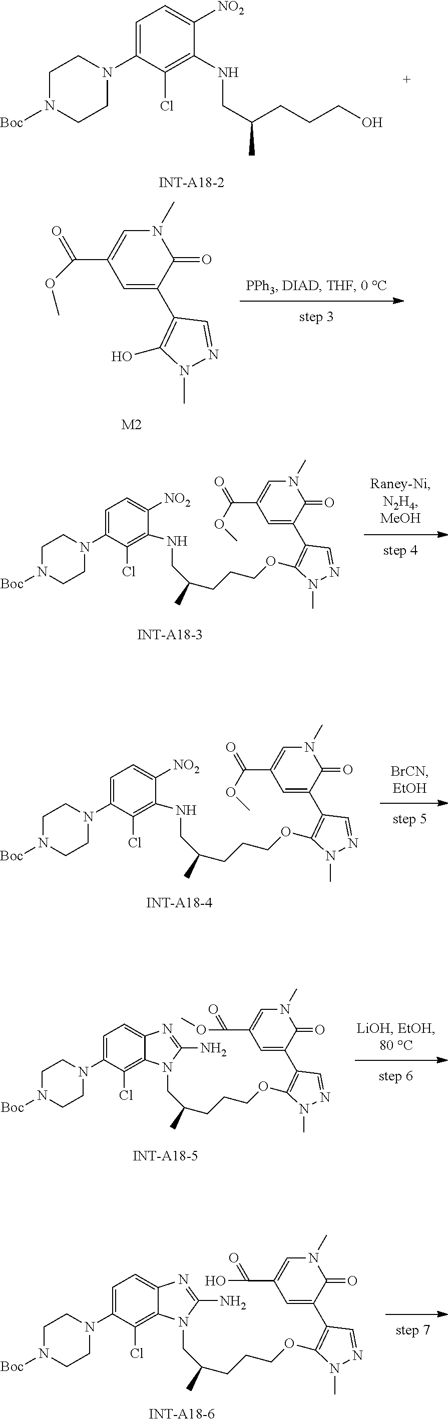

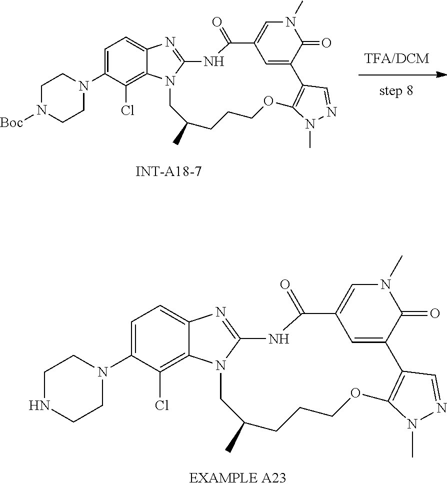 [SMILES: C[C@@H]1CCCOc2c(cnn2C)-c2cc(cn(C)c2=O)C(=O)Nc2nc3ccc(N4CCN(C(=O)OC(C)(C)C)CC4)c(Cl)c3n2C1 ; C[C@@H]1CCCOc2c(cnn2C)-c2cc(cn(C)c2=O)C(=O)Nc2nc3ccc(N4CCNCC4)c(Cl)c3n2C1]

Step 1. Preparation of (4R)-5-[(2-chloro-3-fluoro-6-nitrophenyl)amino]-4-methylpentan-1-ol (INT-A23-1). To a stirred mixture of 2-chloro-1,3-difluoro-4-nitrobenzene (1.73 g, 8.96 mmol, 1.05 equiv) and (4R)-5-amino-4-methylpentan-1-ol (M3-6, 1 g, 8.53 mmol, 1 equiv) in acetonitrile (25 mL) was added K₂CO₃ (2.36 g, 17.06 mmol, 2 equiv) in portions at room temperature. The mixture was stirred for 2 h at 60 °C under nitrogen atmosphere and cooled to room temperature. The reaction was quenched with water (100 mL) and the mixture was extracted with CH₂Cl₂ (3×100 mL). The combined organic layers were washed with brine (3×50 mL), dried over anhydrous Na₂SO₄, and concentrated under reduced pressure. The residue was purified by silica gel chromatography eluted with petroleum ether/ethyl acetate (3/1) to give 2 g of (4R)-5-[(2-chloro-3-fluoro-6-nitrophenyl)amino]-4-methylpentan-1-ol (INT-A23-1) as a yellow oil (80%). LCMS: m/z (ESI), [M+H]⁺=290.95. ¹H NMR (DMSO-d₆, 400 MHz) δ 0.82 (3H, d), 1.01-1.30 (2H, m), 1.31-1.72 (3H, m), 2.78-2.89 (1H, m), 2.94-3.09 (1H, m), 3.33 (2H, d), 4.37 (1H, t), 6.59 (1H, t), 7.22 (1H, d), 7.76 (1H, d).

Step 2. Preparation of tert-butyl 4-(2-chloro-3-{[(2R)-5-hydroxy-2-methylpentyl]amino}-4-nitrophenyl)piperazine-1-carboxylate (INT-A23-2). To a stirred mixture of (4R)-5-[(2-chloro-3-fluoro-6-nitrophenyl)amino]-4-methylpentan-1-ol (2 g, 6.87 mmol, 1 equiv) and tert-butyl piperazine-1-carboxylate (2.56 g, 13.75 mmol, 2 equiv) in dimethylsulfoxide (30 mL) was added N,N-diisopropylethylamine (1.78 g, 13.75 mmol, 2 equiv) dropwise at room temperature. The mixture was stirred for 4 h at 120 °C under nitrogen atmosphere and cooled to room temperature. The reaction was quenched with water (50 mL) and the mixture was extracted with ethyl acetate (3×50 mL). The combined organic layers were washed with brine (3×50 mL), dried over anhydrous Na₂SO₄, and concentrated under reduced pressure. The residue was purified by silica gel chromatography eluted with petroleum ether/ethyl acetate (10/1) to give 3.1 g of tert-butyl 4-(2-chloro-3-{[(2R)-5-hydroxy-2-methylpentyl]amino}-4-nitrophenyl)piperazine-1-carboxylate (INT-A23-2) as a yellow oil (98%). LCMS: m/z (ESI), [M+H]⁺=457.15.

Step 3. Preparation of tert-butyl 4-(2-chloro-3-{[(2R)-5-({4-[5-(methoxycarbonyl)-1-methyl-2-oxopyridin-3-yl]-2-methylpyrazol-3-yl}oxy)-2-methylpentyl]amino}-4-nitrophenyl)piperazine-1-carboxylate (INT-A23-3). To a stirred mixture of methyl 5-(5-hydroxy-1-methylpyrazol-4-yl)-1-methyl-6-oxopyridine-3-carboxylate (M2, 576.04 mg, 1.09 mmol, 1 equiv) and triphenylphosphine (1.7 g, 3.28 mmol, 3 equiv) in tetrahydrofuran (10 mL) were added diisopropylazodicarboxylate (1.3 g, 3.28 mmol, 3 equiv) and tert-butyl 4-(2-chloro-3-{[(2R)-5-hydroxy-2-methylpentyl]amino}-4-nitrophenyl)piperazine-1-carboxylate (1 g, 1.09 mmol, 1 equiv) at 0 °C. The mixture was stirred for 1 h at 0 °C under nitrogen atmosphere. The reaction was quenched with water (50 mL) and the mixture was extracted with ethyl acetate (3×50 mL). The combined organic layers were washed with brine (3×50 mL), dried over anhydrous Na₂SO₄, and concentrated under reduced pressure. The residue was purified by Prep-TLC and eluted with dichloromethane/methanol (25/1) to give 1.1 g of tert-butyl 4-(2-chloro-3-{[(2R)-5-({4-[5-(methoxycarbonyl)-1-methyl-2-oxopyridin-3-yl]-2-methylpyrazol-3-yl}oxy)-2-methylpentyl]amino}-4-nitrophenyl)piperazine-1-carboxylate (INT-A23-3) as a yellow oil (66%). LCMS: m/z (ESI), [M+H]⁺=702.15.

Step 4. Preparation of tert-butyl 4-(4-amino-2-chloro-3-{[(2R)-5-({4-[5-(methoxycarbonyl)-1-methyl-2-oxopyridin-3-yl]-2-methylpyrazol-3-yl}oxy)-2-methylpentyl]amino}phenyl)piperazine-1-carboxylate (INT-A23-4). To a stirred mixture of Raney Nickel (500 mg) and tert-butyl 4-(2-chloro-3-{[(2R)-5-({4-[5-(methoxycarbonyl)-1-methyl-2-oxopyridin-3-yl]-2-methylpyrazol-3-yl}oxy)-2-methylpentyl]amino}-4-nitrophenyl)piperazine-1-carboxylate (1.1 g, 1.56 mmol, 1 equiv) in methanol (10 mL) was added NH₂NH₂—H₂O (235.26 mg, 4.69 mmol, 3 equiv) at 0 °C under nitrogen atmosphere. The mixture was stirred for 1 h at 0 °C and filtered. The filter cake was washed with methanol (2×20 mL). The resulting solution was concentrated under reduced pressure. The residue was purified by Prep-TLC and eluted with dichloromethane/methanol (10/1) to give 610 mg of tert-butyl 4-(4-amino-2-chloro-3-{[(2R)-5-({4-[5-(methoxycarbonyl)-1-methyl-2-oxopyridin-3-yl]-2-methylpyrazol-3-yl}oxy)-2-methylpentyl]amino}phenyl)piperazine-1-carboxylate (INT-A23-4) as a light brown oil (52%). LCMS: m/z (ESI), [M+H]⁺=672.20. ¹H NMR (DMSO-d₆, 400 MHz) δ 0.95 (3H, d), 1.95-1.25 (1H, m), 1.36-1.45 (11H, m), 1.59-1.75 (2H, m), 2.65-2.78 (5H, m), 2.81 (1H, d), 3.57 (4H, s), 3.62-3.76 (7H, m), 3.81 (1H, d), 3.92 (2H, t), 5.76 (3H, s), 6.49-6.59 (2H, m), 7.58-7.69 (1H, m), 8.00 (1H, s), 8.14 (1H, d), 8.45 (1H, d).

Step 5. Preparation of tert-butyl 4-{2-amino-4-chloro-3-[(2R)-5-({4-[5-(methoxycarbonyl)-1-methyl-2-oxopyridin-3-yl]-2-methylpyrazol-3-yl}oxy)-2-methylpentyl]-1,3-benzodiazol-5-yl}piperazine-1-carboxylate (INT-A23-5). A mixture of tert-butyl 4-(4-amino-2-chloro-3-{[(2R)-5-({4-[5-(methoxycarbonyl)-1-methyl-2-oxopyridin-3-yl]-2-methylpyrazol-3-yl}oxy)-2-methylpentyl]amino}phenyl)piperazine-1-carboxylate (600 mg, 0.89 mmol, 1 equiv) and BrCN (94.54 mg, 0.89 mmol, 1 equiv) in ethyl alcohol (8 mL) was stirred for 2 h at room temperature under nitrogen atmosphere and concentrated under vacuum. The residue was purified by Prep-TLC and eluted with dichloromethane/methanol (12/1) to give 480 mg of tert-butyl 4-{2-amino-4-chloro-3-[(2R)-5-({4-[5-(methoxycarbonyl)-1-methyl-2-oxopyridin-3-yl]-2-methylpyrazol-3-yl}oxy)-2-methylpentyl]-1,3-benzodiazol-5-yl}piperazine-1-carboxylate (INT-A23-5) as a light brown solid (76%). LCMS: m/z (ESI), [M+H]⁺=697.20. ¹H NMR (DMSO-d₆, 400 MHz) δ 0.01 (5H, s), 1.24 (2H, s), 1.78-1.92 (8H, m), 2.60-2.79 (2H, m), 2.81-2.95 (4H, m), 3.17 (3H, d), 3.57 (3H, s), 3.71 (6H, d), 4.01-4.22 (2H, m), 5.95 (2H, s), 7.04 (2H, s), 7.47 (1H, s), 7.96 (1H, s), 8.12 (1H, d), 8.39 (1H, d).

Step 6 and Step 7. Preparation of tert-butyl 4-[(11R)-15-chloro-5,11,26-trimethyl-23,27-dioxo-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaen-16-yl]piperazine-1-carboxylate (INT-A23-7). A mixture of tert-butyl 4-{2-amino-4-chloro-3-[(2R)-5-({4-[5-(methoxycarbonyl)-1-methyl-2-oxopyridin-3-yl]-2-methylpyrazol-3-yl}oxy)-2-methylpentyl]-1,3-benzodiazol-5-yl}piperazine-1-carboxylate (INT-A23-5, 450 mg, 0.76 mmol, 1 equiv) and LiOH (18 mg, 0.71 mmol, 1.2 equiv) in tetrahydrofuran/H₂O (2 mL/1 mL) was stirred for 2 h at 80 °C under nitrogen atmosphere. The mixture was cooled to room temperature and concentrated under vacuum. To the residue were added N,N,N,N-tetramethyl-O-(7-azabenzotriazol-1-yl)uronium hexafluorophospate (166.96 mg, 0.44 mmol, 1.5 equiv), N,N-diisopropylethylamine (113.51 mg, 0.87 mmol, 3 equiv) and 1,4-dioxane (6 mL). The mixture was stirred for 3 h under nitrogen atmosphere. The mixture was treated with water (10 mL), extracted with ethyl acetate (3×10 mL), dried over anhydrous Na₂SO₄, and concentrated under reduced pressure. The residue was purified by Prep-TLC and eluted with methylene dichloride/methanol (12/1) to give 160 mg of tert-butyl 4-[(11R)-15-chloro-5,11,26-trimethyl-23,27-dioxo-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaen-16-yl]piperazine-1-carboxylate (INT-A23-7) as a light yellow solid (78%). LCMS: m/z (ESI), [M+H]⁺=665.15.

Step 8. Preparation of (11R)-15-chloro-5,11,26-trimethyl-16-(piperazin-1-yl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (Example A23). A mixture of tert-butyl 4-[(11R)-15-chloro-5,11,26-trimethyl-23,27-dioxo-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaen-16-yl]piperazine-1-carboxylate (150 mg, 0.22 mmol, 1 equiv) in trifuoroacetic acid (1 mL) and CH₂Cl₂ (4 mL) was stirred for 1 h at room temperature under nitrogen atmosphere. The mixture was concentrated under reduced pressure. The residue was treated with saturated NaHCO₃ solution and extracted with CH₂Cl₂ (3×10 mL). The combined organic layers were washed with brine (3×10 mL), dried over anhydrous Na₂SO₄, and concentrated under reduced pressure. The residue was purified by Prep-TLC and eluted with methylene dichloride/methanol (12/1) to give 13.5 mg of (11R)-15-chloro-5,11,26-trimethyl-16-(piperazin-1-yl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dioneas (Example A23) a white solid (56%). LCMS: m/z (EST), [M+H]⁺=565.35. ¹H NMR (DMSO-d₆, 400 MHz) δ 1.23-1.32 (3H, m), 1.52-1.59 (2H, m), 1.73 (1H, s), 1.95 (1H, s), 2.12-2.19 (1H, m), 2.89-2.97 (9H, m), 3.62-3.69 (3H, m), 3.71-3.76 (3H, m), 3.90 (1H, s), 4.19 (1H, m), 4.32-4.39 (1H, m), 4.70-4.79 (1H, m), 7.09-7.27 (1H, m), 7.50 (1H, d), 8.28-8.45 (2H, m), 8.65-8.76 (1H, m).

### Example A25

### (11R)-5,11,26-Trimethyl-16-(4-methylpiperazin-1-yl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octacene-23,27-dione

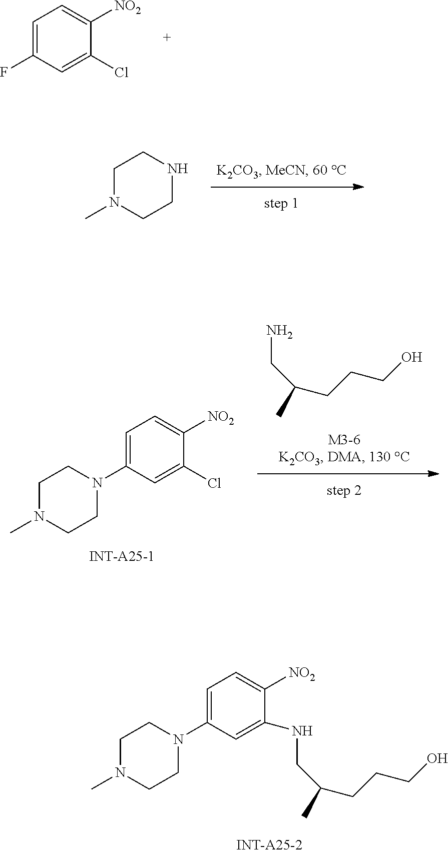

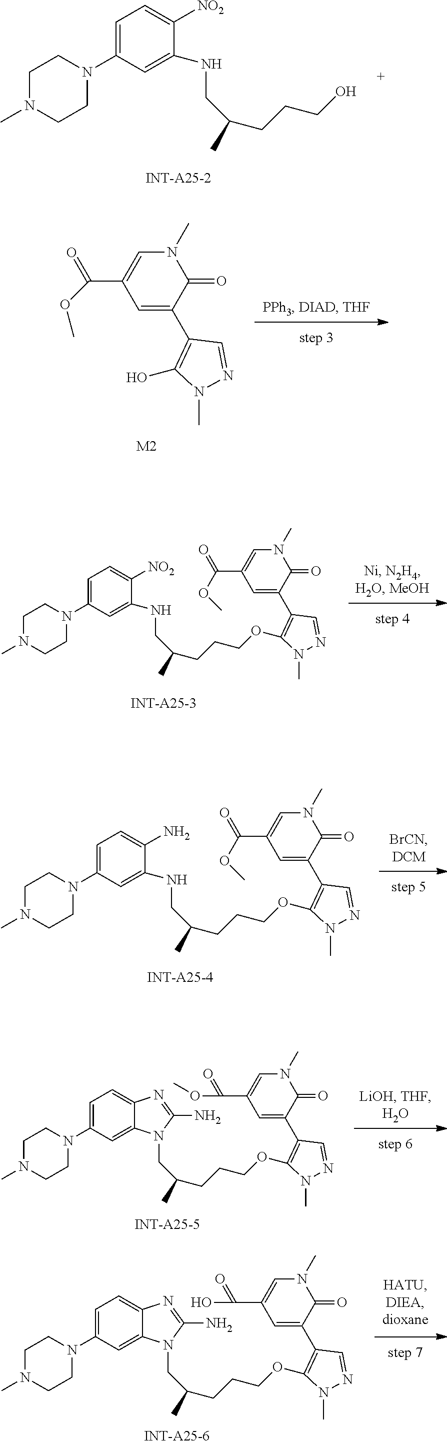

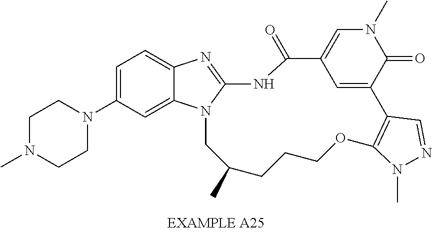 [SMILES: C[C@@H]1CCCOc2c(cnn2C)-c2cc(cn(C)c2=O)C(=O)Nc2nc3ccc(N4CCN(C)CC4)cc3n2C1]

Step 1. Preparation of 1-(3-chloro-4-nitrophenyl)-4-methylpiperazine (INT-A25-1). A mixture of 2-chloro-4-fluoro-1-nitrobenzene (4 g, 22.79 mmol, 1 equiv) and 1-methylpiperazine- (2.28 g, 22.79 mmol, 1 equiv), and K₂CO₃ (6.3 g, 45.57 mmol, 2 equiv) in acetonitrile (60 mL) was stirred for 2 h at 60 °C under nitrogen atmosphere. The mixture was cooled to room temperature, treated with water (100 mL), and extracted with dichloromethane (3×100 mL). The combined organic layers were washed with brine (3×100 mL), dried over anhydrous Na₂SO₄, and concentrated under reduced pressure. The residue was purified by Prep-TLC eluted with petroleum ether/ethyl acetate (1/1) to give 5.6 g of 1-(3-chloro-4-nitrophenyl)-4-methylpiperazine (INT-A25-1) as a yellow solid (96%). LCMS: m/z (EST), [M+H]⁺=256.05. ¹H NMR (DMSO-d₆, 400 MHz) δ 2.22 (3H, s), 2.41 (4H, t), 3.45 (4H, t), 7.00-7.03 (1H, m), 7.10 (1H, d), 8.02 (1H, d).

Step 2. Preparation of (4R)-4-methyl-5-{[5-(4-methylpiperazin-1-yl)-2-nitrophenyl]amino}pentan-1-ol (INT-A25-1). A mixture of 1-(3-chloro-4-nitrophenyl)-4-methylpiperazine (1.5 g, 5.87 mmol, 1 equiv), K₂CO₃ (1.62 g, 11.73 mmol, 2 equiv) and (4R)-5-amino-4-methylpentan-1-ol (M3-6, 0.82 g, 7.04 mmol, 1.2 equiv) in N,N-dimethylformamide (10 mL) was stirred for 16 h at 120 °C under nitrogen atmosphere. The reaction mixture was cooled to room temperature, treated with water (80 mL), and extracted with dichloromethane (3×70 mL). The combined organic layers were washed with brine (3×500 mL), dried over anhydrous Na₂SO₄, and concentrated under reduced pressure. The residue was purified by prep-TLC eluted with petroleum ether/ethyl acetate (1/1) to give 1.5 g of (4R)-4-methyl-5-{[5-(4-methylpiperazin-1-yl)-2-nitrophenyl]amino}pentan-1-ol (INT-A25-2) as a yellow oil (76%). LCMS: m/z (ESI), [M+H]⁺=337.15.

Step 3. Preparation of methyl 1-methyl-5-(1-methyl-5-{[(4R)-4-methyl-5-{[5-(4-methylpiperazin-1-yl)-2-nitrophenyl]amino}pentyl]oxy}pyrazol-4-yl)-6-oxopyridine-3-carboxylate (INT-A25-3). To a stirred mixture of triphenylphosphine (1.57 g, 5.97 mmol, 3 equiv) and methyl 5-(5-hydroxy-1-methylpyrazol-4-yl)-1-methyl-6-oxopyridine-3-carboxylate (M2, 498.05 mg, 1.89 mmol, 0.95 equiv) in tetrahydrofuran (20 mL) were added (4R)-4-methyl-5-{[5-(4-methylpiperazin-1-yl)-2-nitrophenyl]amino}pentan-1-ol (670 mg, 1.99 mmol, 1 equiv) and diisopropyl azodicarboxylate (1.21 g, 5.97 mmol, 3 equiv) at 0 °C under nitrogen atmosphere. The mixture was stirred for 2 h at room temperature, treated with water (80 mL), and extracted with ethyl acetate (3×80 mL). The combined organic layers were washed with water (3×50 mL), dried over anhydrous Na₂SO₄, and concentrated under reduced pressure. The residue was purified on silica gel column eluted with dichloromethane/methanol (35/1) to give 500 mg of methyl 1-methyl-5-(1-methyl-5-{[(4R)-4-methyl-5-{[5-(4-methylpiperazin-1-yl)-2-nitrophenyl]amino}pentyl]oxy}pyrazol-4-yl)-6-oxopyridine-3-carboxylate (INT-A25-3) as a light yellow solid (43%). LCMS: m/z (ESI), [M+H]⁺=582.20.

Step 4. Preparation of methyl 5-(5-{[(4R)-5-{[2-amino-5-(4-methylpiperazin-1-yl)phenyl]amino}-4-methylpentyl]oxy}-1-methylpyrazol-4-yl)-1-methyl-6-oxopyridine-3-carboxylate (INT-A25-4). To a stirred mixture of methyl 1-methyl-5-(1-methyl-5-{[(4R)-4-methyl-5-{[5-(4-methylpiperazin-1-yl)-2-nitrophenyl]amino}pentyl]oxy}pyrazol-4-yl)-6-oxopyridine-3-carboxylate (700 mg, 1.20 mmol, 1 equiv) in methanol (6 mL) were added Raney Nickel (349.52 mg, 4.08 mmol, 3.4 equiv) and NH₂NH₂—H₂O (90.37 mg, 1.80 mmol, 1.5 equiv) at 0 °C under nitrogen atmosphere. The resulting mixture was stirred for 1 h at room temperature, treated with water (20 mL), and extracted with dichloromethane (3×20 mL). The combined organic layers were washed with water (3×10 mL), dried over anhydrous Na₂SO₄, and concentrated under reduced pressure to give 510 mg methyl 5-(5-{[(4R)-5-{[2-amino-5-(4-methylpiperazin-1-yl)phenyl]amino}-4-methylpentyl]oxy}-1-methylpyrazol-4-yl)-1-methyl-6-oxopyridine-3-carboxylate (INT-A25-4) as a light yellow solid (77%). This material was used for the next reaction without further purification. LCMS: m/z (EST), [M+H]⁺=552.25.

Step 5. Preparation of methyl 5-(5-{[(4R)-4-{[2-amino-6-(4-methylpiperazin-1-yl)-1,3-benzodiazol-1-yl]methyl}pentyl]oxy}-1-methylpyrazol-4-yl)-1-methyl-6-oxopyridine-3-carboxylate (INT-A25-5). To a stirred mixture of methyl 5-(5-{[(4R)-5-{[2-amino-5-(4-methylpiperazin-1-yl)phenyl]amino}-4-methylpentyl]oxy}-1-methylpyrazol-4-yl)-1-methyl-6-oxopyridine-3-carboxylate (480 mg, 0.87 mmol, 1 equiv) in dichloromethane (8 mL) was added cyanogen bromide (138.24 mg, 1.30 mmol, 1.5 equiv) at room temperature under nitrogen atmosphere. The resulting mixture was stirred for 2 h at room temperature under nitrogen atmosphere, treated with water (20 mL), and extracted with dichloromethane (3×20 mL). The combined organic layers were washed with brine (3×50 mL), dried over anhydrous Na₂SO₄, and concentrated under reduced pressure. The residue was purified by silica gel chromatography eluted with dichloromethane/methanol (15/1) to give 170 mg of methyl 5-(5-{[(4R)-4-{[2-amino-6-(4-methylpiperazin-1-yl)-1,3-benzodiazol-1-yl]methyl}pentyl]oxy}-1-methylpyrazol-4-yl)-1-methyl-6-oxopyridine-3-carboxylate (INT-A25-5) as a purple solid (34%). LCMS: m/z (ESI), [M+H]⁺=577.40.

Step 6. Preparation of 5-(5-{[(4R)-4-{[2-amino-6-(4-methylpiperazin-1-yl)-1,3-benzodiazol-1-yl]methyl}pentyl]oxy}-1-methylpyrazol-4-yl)-1-methyl-6-oxopyridine-3-carboxylic acid (INT-A25-6). To a stirred mixture of methyl 5-(5-{[(4R)-4-{[2-amino-6-(4-methylpiperazin-1-yl)-1,3-benzodiazol-1-yl]methyl}pentyl]oxy}-1-methylpyrazol-4-yl)-1-methyl-6-oxopyridine-3-carboxylate (140 mg, 0.24 mmol, 1 equiv) in tetrahydrofuran (4 mL) was added LiOH (11.63 mg, 0.49 mmol, 2 equiv) in water (0.3 mL) at room temperature. The resulting mixture was stirred for 1 h at room temperature and concentrated under reduced pressure. The residue was purified by reverse flash chromatography eluted with acetonitrile in water (0% to 100%) to give 37 mg of 5-(5-{[(4R)-4-{[2-amino-6-(4-methylpiperazin-1-yl)-1,3-benzodiazol-1-yl]methyl}pentyl]oxy}-1-methylpyrazol-4-yl)-1-methyl-6-oxopyridine-3-carboxylic acid (INT-A25-6) as a white solid (27%). LCMS: m/z (ESI), [M+H]⁺=563.35.

Step 7. Preparation of (11R)-5,11,26-trimethyl-16-(4-methylpiperazin-1-yl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (EXAMPLE A25). To a stirred mixture of 5-(5-{[(4R)-4-{[2-amino-6-(4-methylpiperazin-1-yl)-1,3-benzodiazol-1-yl]methyl}pentyl]oxy}-1-methylpyrazol-4-yl)-1-methyl-6-oxopyridine-3-carboxylic acid (34 mg, 0.06 mmol, 1 equiv) in dioxane (3 mL) were added N,N-diisopropylethylamine (23 mg, 0.18 mmol, 3 equiv) and N,N,N,N-tetramethyl-O-(7-azabenzotriazol-1-yl)uronium hexafluorophospate (34 mg, 0.09 mmol, 1.5 equiv) at room temperature. The resulting mixture was stirred for 1 h at 60 °C under nitrogen atmosphere, cooled to room temperature, and concentrated under reduced pressure. The residue was purified by Prep-HPLC with XBridge Shield RP18 OBD column using water containing 10 mmol/L NH₄HCO₃ and acetonitrile as mobile phase. Fractions containing the desired compound were evaporated to dryness to give 14.4 mg of (11R)-5,11,26-trimethyl-16-(4-methylpiperazin-1-yl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (EXAMPLE A25) as a white solid (43%). LCMS: m/z (ESI), [M+H]⁺=545.35. ¹H NMR (DMSO-d₆, 400 MHz) δ 0.81 (3H, d), 1.44 (1H, q), 1.92 (2H, d), 2.12-2.25 (1H, m), 2.25 (3H, s), 2.50 (4H, t), 2.80 (1H, br s), 3.16 (4H, t), 3.62 (3H, s), 3.72 (3H, s), 3.88-3.99 (2H, m), 4.10 (1H, d), 4.33-4.38 (1H, m), 6.86 (1H, dd), 7.13 (1H, d), 7.36 (1H, d), 8.27 (1H, d), 8.36 (1H, s), 8.81 (1H, d), 12.35 (1H, s).

### Example A26

### (11R) 5,11,26-trimethyl-16-(morpholin-4-yl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione

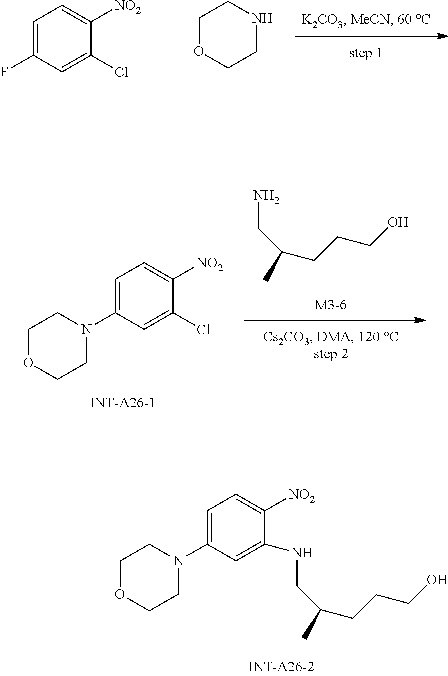

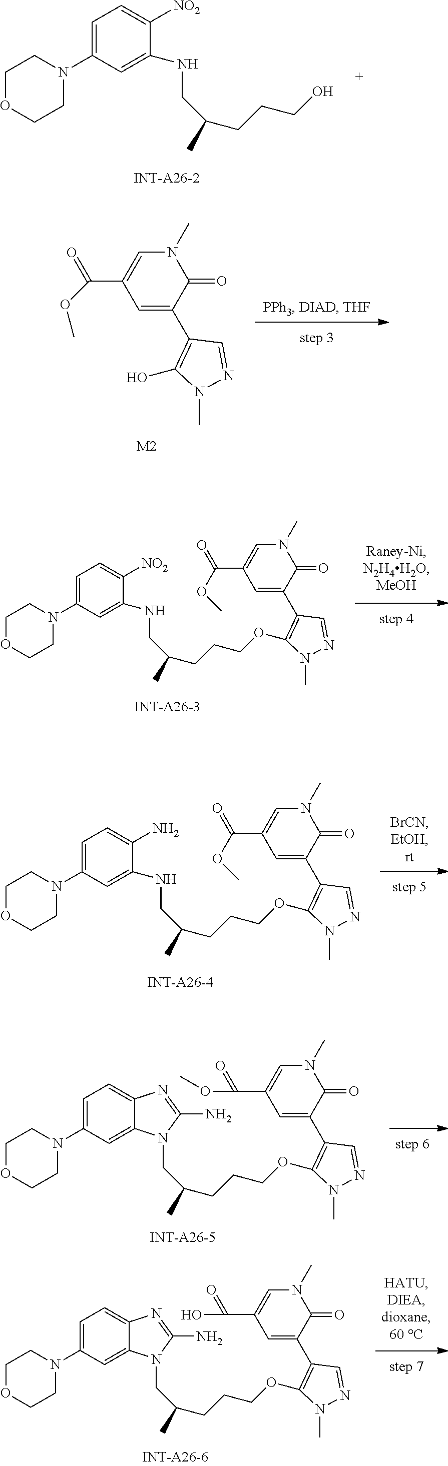

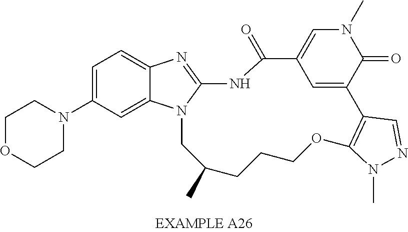 [SMILES: C[C@@H]1CCCOc2c(cnn2C)-c2cc(cn(C)c2=O)C(=O)Nc2nc3ccc(N4CCOCC4)cc3n2C1]

Step 1. Preparation of 4-(3-chloro-4-nitrophenyl)morpholine (INT-A26-1). A mixture of 2-chloro-4-fluoro-1-nitrobenzene (3 g, 17.09 mmol, 1 equiv), K₂CO₃ (4.72 g, 34.20 mmol, 2 equiv), and morpholine (1.94 g, 22.20 mmol, 1.3 equiv) in acetonitrile (30 mL) was stirred for 4 h at 80 °C under nitrogen atmosphere. The mixture was cooled to room temperature, treated water (100 mL), and extracted with ethyl acetate (3×100 mL). The combined organic layers were washed with brine (3×100 mL), dried over anhydrous Na₂SO₄, and concentrated under reduced pressure. The residue was purified on silica gel column eluted with petroleum ether/ethyl acetate (5/1) to give 2.7 g of 4-(3-chloro-4-nitrophenyl)morpholine (INT-A26-1) as a yellow solid (65%). ¹H NMR (DMSO-d_{d}, 400 MHz) δ 3.38-3.49 (4H, m), 3.63-3.75 (4H, m,), 7.00-7.04 (1H, m), 7.12 (1H, d,), 8.04 (1H, d,).

Step 2. Preparation of (R)-4-methyl-5-((5-morpholino-2-nitrophenyl)amino)pentan-1-ol (INT-A26-2). A mixture of 4-(3-chloro-4-nitrophenyl)morpholine (1 g, 4.12 mmol, 1 equiv), Cs₂CO₃ (2.69 g, 8.24 mmol, 2 equiv), and (4R)-5-amino-4-methylpentan-1-ol (0.58 g, 4.94 mmol, 1.2 equiv) in dimethylacetamide (15 mL) was stirred for 4 h at 120 °C under nitrogen atmosphere and cooled to room temperature. The reaction was quenched with water (100 mL) and the mixture was extracted with ethyl acetate (3×100 mL). The combined organic layers were washed with brine (3×100 mL), dried over anhydrous Na₂SO₄, and concentrated under reduced pressure. The residue was purified by reverse flash chromatography with C18 silica gel column using acetonitrile in water (0% to 100% gradient) as mobile phase to give 730 mg of (4R)-4-methyl-5-{[5-(morpholin-4-yl)-2-nitrophenyl]amino}pentan-1-ol (INT-A26-2) as a brown solid (54%). LCMS: m/z (ESI), [M+H]⁺=324.20. ¹HNMR (DMSO-d₆, 400 MHz) δ 0.97 (3H, d), 1.21-1.29 (1H, m), 1.40-1.47 (3H, m), 1.78-1.87 (1H, m), 3.09-3.19 (1H, m), 3.21-3.28 (1H, m), 3.45-3.50 (4H, m) 3.69-3.73 (6H, m), 4.40 (1H, s), 6.40-6.43 (1H, m), 7.00-7.10 (1H, m), 7.92 (1H, d), 8.02-8.12 (1H, m).

Step 3. Preparation of methyl (R)-1-methyl-5-(1-methyl-5-((4-methyl-5-((5-morpholino-2-nitrophenl)amino)pentyl)oxy)-1H-pyrazol-4-yl)-6-oxo-1,6-dihydropyridine-3-carboxylate (INT-A26-3). To a stirred mixture of methyl 5-(5-hydroxy-1-methylpyrazol-4-yl)-1-methyl-6-oxopyridine-3-carboxylate (M2, 260 mg, 0.98 mmol, 1 equiv) and triphenylphosphine (777 mg, 2.964 mmol, 3.0 equiv) in tetrahydrofuran (3 mL) were added diisopropylazodicarboxylate (599 mg, 2.96 mmol, 3 equiv) and (4R)-4-methyl-5-{[5-(morpholin-4-yl)-2-nitrophenyl]amino}pentan-1-ol (638 mg, 1.97 mmol, 2 equiv) at 0 °C. The mixture was stirred for 2 h at 0 °C under nitrogen atmosphere, treated with water (100 mL), and extracted with ethyl acetate (3×100 mL). The combined organic layers were washed with brine (3×100 mL), dried over anhydrous Na₂SO₄, and concentrated under reduced pressure. The residue was purified by Prep-TLC and eluted with petroleum ether/ethyl acetate (1/1) to give 130 mg of methyl 1-methyl-5-(1-methyl-5-{[(4R)-4-methyl-5-{[5-(morpholin-4-yl)-2-nitrophenyl]amino}pentyl]oxy}pyrazol-4-yl)-6-oxopyridine-3-carboxylate (INT-A26-3) as a brown oil (23%). LCMS: m/z (ESI), [M+H]⁺=569.30. ¹H NMR (DMSO-d₆, 400 MHz) δ 0.98 (3H, d), 1.31-1.45 (1H, m), 1.60 (1H, m), 1.74-1.92 (3H, m), 3.16 (1H, m), 3.23-3.30 (1H, m), 3.37-3.43 (4H, m), 3.57 (3H, s), 3.65-3.73 (7H, m), 3.77 (3H, s), 3.96 (2H, t), 6.01 (1H, d), 6.43-6.48 (1H, m), 7.92 (1H, d), 7.99 (1H, s), 8.14 (1H, d), 8.41-8.46 (2H, m).

Step 4. Preparation of methyl (R)-5-(5-((5-((2-amino-5-morpholinophenyl)amino)-4-methylpentyl)oxy)-1-methyl-1H-pyrazol-4-yl)-1-methyl-6-oxo-1,6-dihydropyridine-3-carboxylate (INT-A26-4). To a stirred mixture of Raney Nickel (40 mg, 0.47 mmol, 1 equiv) and methyl 1-methyl-5-(1-methyl-5-{[(4R)-4-methyl-5-{[5-(morpholin-4-yl)-2-nitrophenyl]amino}pentyl]oxy}pyrazol-4-yl)-6-oxopyridine-3-carboxylate (INT-A26-3, 270 mg, 0.47 mmol, 1 equiv) in methanol (4 mL) was added NH₂NH₂—H₂O (47 mg, 0.95 mmol, 2 equiv) at 0 °C under nitrogen atmosphere. The mixture was stirred for 1 h at 0 °C and filtered. The filter cake was washed with methanol (2×50 mL) and the resulting solution was concentrated under reduced pressure. The crude product was used in the next step directly without further purification. LCMS: m/z (ESI), [M+H]⁺=539.30.

Step 5. Preparation of methyl(R)-5-(5-((5-(2-amino-6-morpholino-1H-benzo[d]imidazol-1-yl)-4-methylpentyl)oxy)-1-methyl-1H-pyrazol-4-yl)-1-methyl-6-oxo-1,6-dihydropyridine-3-carboxylate (INT-A26-5). A mixture of methyl 5-(5-{[(4R)-5-{[2-amino-5-(morpholin-4-yl)phenyl]amino}-4-methylpentyl]oxy}-1-methylpyrazol-4-yl)-1-methyl-6-oxopyridine-3-carboxylate (190 mg, 0.35 mmol, 1 equiv) and BrCN (41 mg, 0.38 mmol, 1.1 equiv) in ethyl alcohol (3 mL) was stirred for 2 h at room temperature under nitrogen atmosphere. The mixture was concentrated under vacuum. The residue was purified by Prep-TLC and eluted with dichloromethane/methanol (12/1) to give 100 mg of methyl 5-(5-{[(4R)-4-{[2-amino-6-(morpholin-4-yl)-1,3-benzodiazol-1-yl]methyl}pentyl]oxy}-1-methylpyrazol-4-yl)-1-methyl-6-oxopyridine-3-carboxylate (INT-A26-5) as a pink solid (50%). ¹H NMR (DMSO-d₆, 400 MHz) δ 0.84 (d, 3H), 1.27-1.40 (1H, m), 1.44-1.60 (1H, m), 1.67-1.71 (1H, m), 1.80-1.94 (1H, m), 2.04 (1H, s), 2.94-3.03 (3H, m), 3.17 (1H, d), 3.55-3.61 (5H, m), 3.69-3.76 (4H, m), 3.78 (3H, s), 3.88-3.91 (2H, m), 6.21 (1H, s), 6.59-6.63 (1H, m), 6.76 (2H, d), 7.00 (1H, d), 7.99 (1H, s), 8.13 (1H, d), 8.45 (1H, d).

Step 6 Preparation of (R)-5-(5-((5-(2-amino-6-morpholino-1H-benzo[d]imidazol-1-yl)-4-methylpentyl)oxy)-1-methyl-1H-pyrazol-4-yl)-1-methyl-6-oxo-1,6-dihydropyridine-3-carboxylic acid (INT-A26-6). A mixture of methyl 5-(5-{[(4R)-4-{[2-amino-6-(morpholin-4-yl)-1,3-benzodiazol-1-yl]methyl}pentyl]oxy}-1-methylpyrazol-4-yl)-1-methyl-6-oxopyridine-3-carboxylate (100 mg, 0.17 mmol, 1 equiv) and LiOH (8.5 mg, 0.35 mmol, 2 equiv) in tetrahydrofuran/H₂O (4 mL/1 mL) was stirred for 2 h at room temperature under nitrogen atmosphere and concentrated under vacuum. The residue was purified by reverse flash chromatography using C18 silica gel column and acetonitrile in water (0% to 100% gradient) as mobile phase to give 85 mg of 5-(5-{[(4R)-4-{[2-amino-6-(morpholin-4-yl)-1,3-benzodiazol-1-yl]methyl}pentyl]oxy}-1-methylpyrazol-4-yl)-1-methyl-6-oxopyridine-3-carboxylic acid (INT-A26-6) as a brown solid (87%). ¹H NMR (DMSO-dd, 400 MHz) δ 0.78 (3H, d), 1.43 (1H, s), 1.54-1.75 (2H, m), 1.83 (1H, d), 2.03 (1H, s), 2.89-3.04 (4H, m), 3.50 (3H, s), 3.60 (3H, s), 3.70-3.75 (4H, m), 3.79-3.82 (1H, m), 3.94 (3H, m), 6.56-6.62 (2H, m), 6.76 (1H, d), 6.96 (1H, d), 8.00 (2H, d), 8.01-8.03 (1H, s), 8.31 (1H, d).

Step 7 Preparation of (11R)5,11,26-trimethyl-16-(morpholin-4-yl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (EXAMPLE A26). A mixture of 5-(5-{[(4R)-4-{[2-amino-6-(morpholin-4-yl)-1,3-benzodiazol-1-yl]methyl}pentyl]oxy}-1-methylpyrazol-4-yl)-1-methyl-6-oxopyridine-3-carboxylic acid (50 mg, 0.09 mmol, 1 equiv), N,N-diisopropylethylamine (35 mg, 0.27 mmol, 3 equiv) and N,N,N,N-tetramethyl-O-(7-azabenzotriazol-1-yl)uronium hexafluorophospate (51 mg, 0.13 mmol, 1.5 equiv) in 1,4-dioxane (3 mL) was stirred for 1 h at 60 °C under nitrogen atmosphere. The mixture was cooled to room temperature and concentrated under vacuum. The residue was purified by Prep-HPLC with XBridge Prep OBD C18 Column using water containing 10 mmol/L NH₄HCO₃ and acetonitrile as mobile phase. Fractions containing the desired compound were evaporated to dryness to give 16 mg of (11R)5,11,26-trimethyl-16-(morpholin-4-yl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (EXAMPLE A26) as a white solid (34%). LCMS: m/z (ESI), [M+H]+=532.30. ¹H NMR (DMSO-d₆, 400 MHz) δ 0.81 (3H, d), 1.43 (1H, q), 1.92 (2H, d), 2.19 (1H, br s), 2.80 (1H, br s), 3.14 (4H, t), 3.62 (3H, s), 3.72 (3H, s), 3.78 (4H, t), 3.89-3.97 (2H, m), 4.06-4.14 (1H, m), 4.31-4.41 (1H, m), 6.87 (1H, dd), 7.15 (1H, d), 7.38 (1H, d), 8.27 (1H, d), 8.35 (1H, s), 8.81 (1H, d), 12.37 (1H, s).

### Example A27

### (11R)-16-[3-(Dimethylamino)pyrrolidin-1-yl]-5,11,26-trimethyl-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione

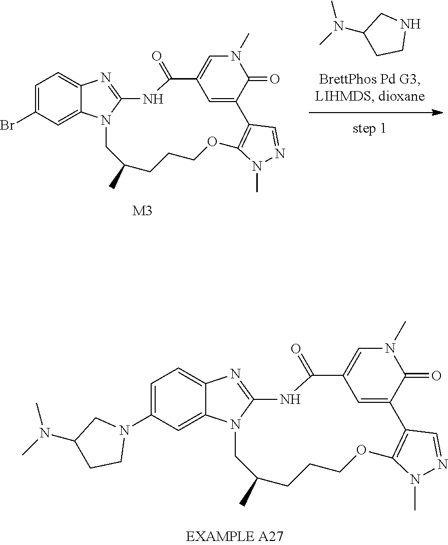

Step 1. Preparation of (11R)-16-[3-(dimethylamino)pyrrolidin-1-yl]-5,11,26-trimethyl-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (EXAMPLE A27). To a stirred mixture of (11R)-16-bromo-5,11,26-trimethyl-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (M3, 40 mg, 0.07 mmol, 1 equiv), N,N-dimethylpyrrolidin-3-amine (34 mg, 0.30 mmol, 4 equiv), and BrettPhos Pd G₃ (20 mg, 0.02 mmol, 0.3 equiv) in 1,4-dioxane (5 mL) was added LiHMDS (0.22 mL, 0.22 mmol, 3 equiv) dropwise at room temperature under nitrogen atmosphere. The reaction mixture was stirred for 30 min at 60 °C. After being cooled to room temperature, the reaction was quenched with sat. NH₄Cl solution (30 mL) and the mixture was extracted with dichloromethane (3×20 mL). The combined organic layers were washed with brine (3×30 mL), dried over anhydrous Na₂SO₄, and concentrated under reduced pressure. The residue was purified by prep-TLC eluted with dichloromethane/methanol (15/1) and the product was further purified by Prep-HPLC with XBridge Prep OBD C18 column using wate containing 10 mmol/L NH₄HCO₃ and acetonitrile as mobile phase. Fractions containing the desired compound were evaporated to dryness to give 5 mg of (11R)-16-[3-(dimethylamino)pyrrolidin-1-yl]-5,11,26-trimethyl-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (EXAMPLE A27) as a white solid (13%). LCMS: m/z (ESI), [M+H]⁺=559.50. ¹H NMR (DMSO-d₆, 400 MHz) δ 0.81 (3H, d), 1.40-1.50 (1H, m), 1.88-1.95 (3H, m), 2.12-2.31 (8H, m), 2.79-2.84 (2H, m), 3.06-3.17 (1H, m), 3.23-3.49 (3H, m), 3.62 (3H, s), 3.72 (3H, s), 3.84-3.97 (2H, m), 4.06-4.15 (1H, m), 4.32-4.41 (1H, m), 6.47 (1H, d), 6.66 (1H, s), 7.33 (1H, d), 8.26 (1H, d), 8.36 (1H, s), 8.81 (1H, d), 12.28 (1H, s).

The following examples were prepared from M3 using the similar conditions described above for the synthesis of EXAMPLE A27: EXAMPLE A28, EXAMPLE A29, EXAMPLE A30, EXAMPLE A31, EXAMPLE A32, EXAMPLE A33, EXAMPLE A34, EXAMPLE A35, EXAMPLE A36, EXAMPLE A37, EXAMPLE A38, EXAMPLE A29, EXAMPLE A40, EXAMPLE A41, EXAMPLE A42, EXAMPLE A43, EXAMPLE A44, EXAMPLE A45, EXAMPLE A46, EXAMPLE A47, EXAMPLE A48, EXAMPLE A49, EXAMPLE A50, EXAMPLE A51, EXAMPLE A52, EXAMPLE A53, EXAMPLE A54, EXAMPLE A55, EXAMPLE A56, EXAMPLE A57, and EXAMPLE A58.

The structures and the data of m/z and ¹H NMR are listed in Table 1.

### Example A59, Example A60A and Example A60B

### 6-Bromo-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (Example A59)

### 15,21-dimethyl-6-(4-methylpiperazin-1-yl)-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, Isomer 1 (Example A60A) and Isomer 2 (Example A60B)

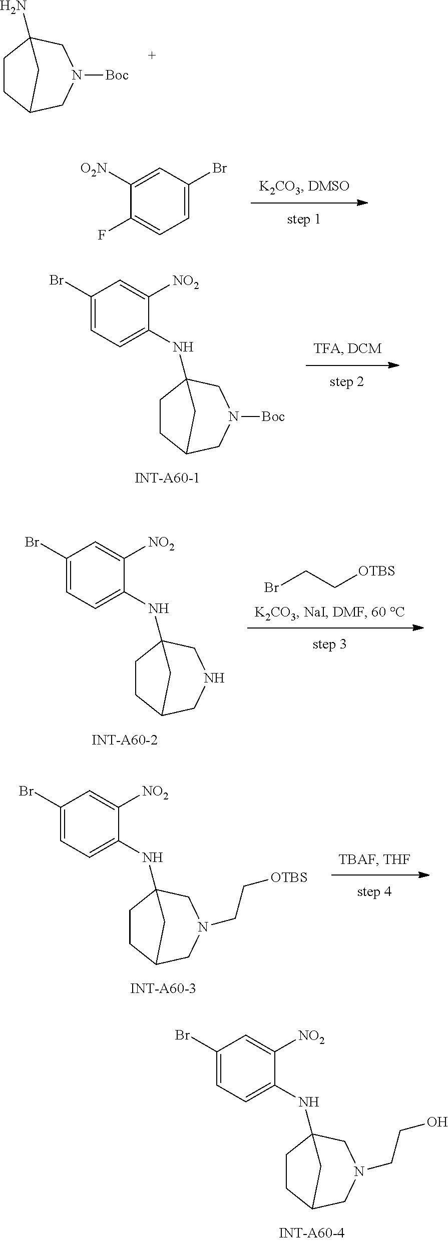

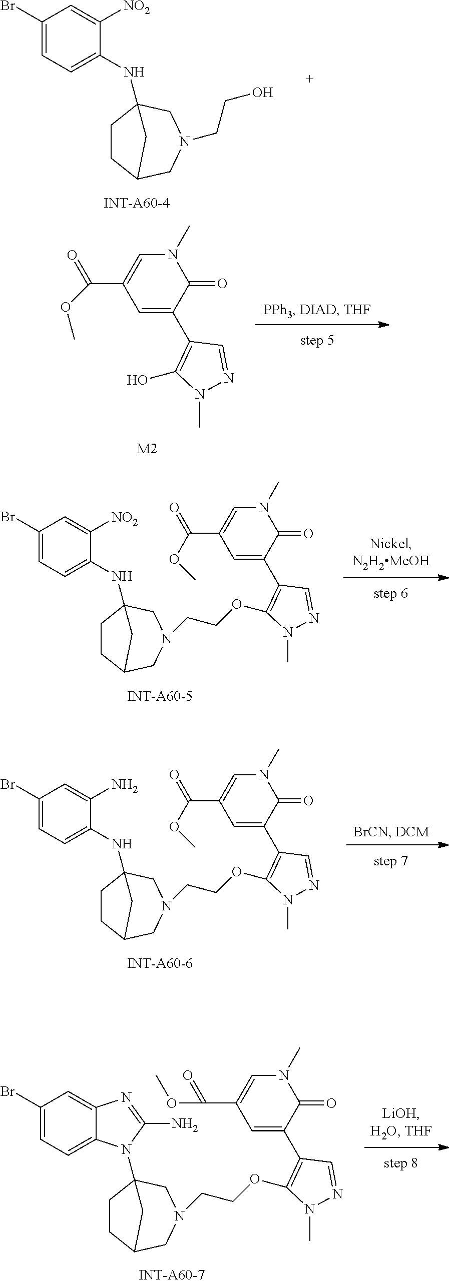 [SMILES: O=[N+]([O-])c1cc(Br)ccc1NC12CCC(CN(CCO)C1)C2 ; COC(=O)c1cc(-c2cnn(C)c2O)c(=O)n(C)c1 ; "M2" ; COC(=O)c1cc(-c2cnn(C)c2OCCN2CC3CCC(Nc4ccc(Br)cc4[N+](=O)[O-])(C3)C2)c(=O)n(C)c1 ; COC(=O)c1cc(-c2cnn(C)c2OCCN2CC3CCC(Nc4ccc(Br)cc4N)(C3)C2)c(=O)n(C)c1 ; COC(=O)c1cc(-c2cnn(C)c2OCCN2CC3CCC(n4c(N)nc5cc(Br)ccc54)(C3)C2)c(=O)n(C)c1]

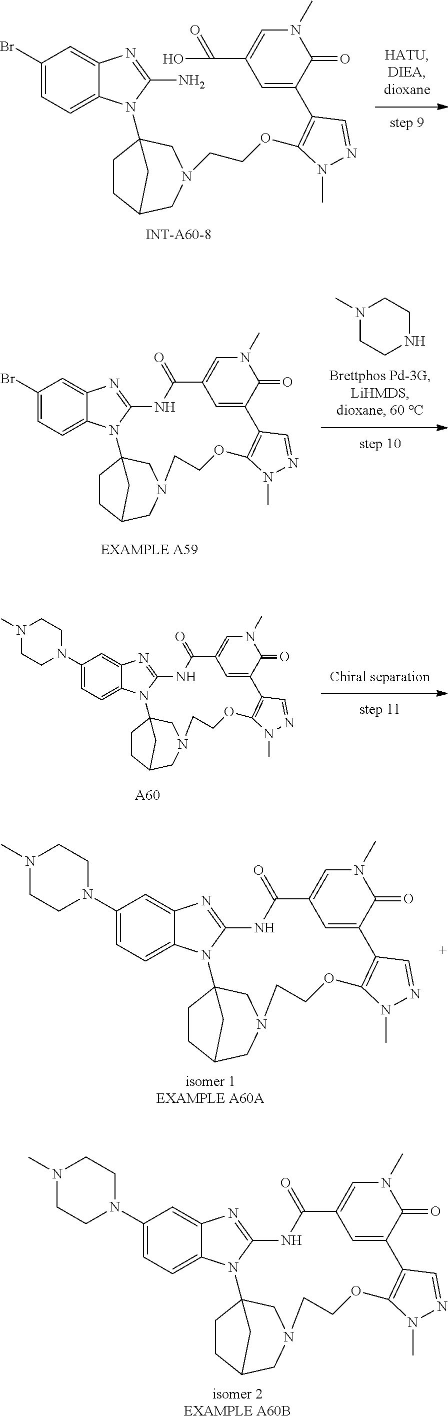

Step 1. Preparation of tert-butyl 1-[(4-bromo-2-nitrophenyl)amino]-3-azabicyclo[3.2.1]octane-3-carboxylate (INT-A60-1). A mixture of tert-butyl 1-amino-3-azabicyclo[3.2.1]octane-3-carboxylate (2.0 g, 8.80 mmol, 1 equiv), K₂CO₃ (2.44 g, 17.6 mmol, 2 equiv) and 4-bromo-1-fluoro-2-nitrobenzene (3.88 g, 17.6 mmol, 2 equiv) in DMSO (40 mL) was stirred for 2 h at 120 °C under nitrogen atmosphere. The mixture was cooled to room temperature. The reaction was quenched with water (200 mL) and the mixture was extracted with ethyl acetate (3×200 mL). The combined organic layers were washed with brine (3×200 mL), dried over anhydrous Na₂SO₄, and concentrated under reduced pressure. The residue was purified by C18 column using water and acetonitrile as mobile phase. Fractions containing the desired compound were evaporated to dryness to give 800 mg of tert-butyl 1-[(4-bromo-2-nitrophenyl)amino]-3-azabicyclo[3.2.1]octane-3-carboxylate (INT-A60-1) as a yellow oil (21%). LCMS: m/z (ESI), [M+H]⁺=372.05.

Step 2. Preparation of N-(4-bromo-2-nitrophenyl)-3-azabicyclo[3.2.1]octan-1-amine (INT-A60-2). A mixture of tert-butyl 1-[(4-bromo-2-nitrophenyl)amino]-3-azabicyclo[3.2.1]octane-3-carboxylate (550 mg, 1.29 mmol, 1 equiv) and trifluoroacetic acid (2 mL) in dichloromethane (6 mL) was stirred for 2 h at room temperature under nitrogen atmosphere, treated with saturated NaHCO₃ (aq.) solution (50 mL), and extracted with CH₂Cl₂ (3×50 mL). The combined organic layers were washed with brine (3×30 mL), dried over anhydrous Na₂SO₄, and concentrated under reduced pressure to give 435 mg of N-(4-bromo-2-nitrophenyl)-3-azabicyclo[3.2.1]octan-1-amine (INT-A60-2) as a yellow solid (100%). LCMS: m/z (ESI), [M+H]⁺=328.00.

Step 3. Preparation of N-(4-bromo-2-nitrophenyl)-3-{2-[(tert-butyldimethylsilyl)oxy]ethyl}-3-azabicyclo[3.2.1]octan-1-amine (INT-A60-3). A mixture of N-(4-bromo-2-nitrophenyl)-3-azabicyclo[3.2.1]octan-1-amine (435 mg, 1.34 mmol, 1 equiv), (2-bromoethoxy)(tert-butyl)dimethylsilane (652 mg, 2.72 mmol, 2 equiv), NaT (408 mg, 2.72 mmol, 2 equiv), and K₂CO₃ (377 mg, 2.72 mmol, 2 equiv) in DMF (5 mL) was stirred for 4 h at 60 °C under nitrogen atmosphere. The mixture was cooled to room temperature, treated with water (50 mL), and extracted with ethyl acetate (3×50 mL). The combined organic layers were washed with brine (3×50 mL), dried over anhydrous Na₂SO₄, and concentrated under reduced pressure. The residue was purified by C18 column using water and acetonitrile as mobile phase. Fractions containing the desired compound were evaporated to dryness to give 360 mg of N-(4-bromo-2-nitrophenyl)-3-{2-[(tert-butyldimethylsilyl)oxy]ethyl}-3-azabicyclo[3.2.1]octan-1-amine (INT-A60-3) as a yellow solid (54%). LCMS: m/z (ESI), [M+H]⁺=485.90.

Step 4. Preparation of 2-{1-[(4-bromo-2-nitrophenyl)amino]-3-azabicyclo[3.2.1]octan-3-yl}ethanol (INT-A60-4). A mixture of N-(4-bromo-2-nitrophenyl)-3-{2-[(tert-butyldimethylsilyl)oxy]ethyl}-3-azabicyclo[3.2.1]octan-1-amine (360 mg, 0.74 mmol, 1 equiv) and tetrabutylammonium fluoride (388 mg, 1.48 mmol, 2 equiv) in tetrahydrofuran (5 mL) was stirred overnight at room temperature under nitrogen atmosphere. The mixture was concentrated under reduced pressure. The residue was loaded onto a silica gel column and eluted with dichloromethane/methanol (10/1) to give 260 mg of 2-{1-[(4-bromo-2-nitrophenyl)amino]-3-azabicyclo[3.2.1]octan-3-yl}ethanol (INT-A60-4) as a yellow solid (94%). LCMS: m/z (ESI), [M+H]⁺=370.10. ¹H NMR (DMSO-dd, 400 MHz) δ 1.61-1.71 (3H, m), 1.79-1.83 (1H, m), 1.91-1.97 (1H, m), 2.11-2.17 (1H, m), 2.18-2.24 (1H, m), 2.30-2.49 (5H, m), 3.11-3.18 (1H, m), 3.45-3.55 (2H, m), 4.34 (1H, t), 7.17 (1H, d), 7.57-7.67 (1H, m), 8.18 (1H, d), 8.29 (1H, s).

Step 5. Preparation of methyl 5-[5-(2-{1-[(4-bromo-2-nitrophenyl)amino]-3-azabicyclo[3.2.1]octan-3-yl}ethoxy)-1-methylpyrazol-4-yl]-1-methyl-6-oxopyridine-3-carboxylate (INT-A60-5). To a stirred mixture of methyl 5-(5-hydroxy-1-methylpyrazol-4-yl)-1-methyl-6-oxopyridine-3-carboxylate (M2, 260 mg, 1.25 mmol, 1.97 equiv) and triphenylphosphine (782 mg, 2.98 mmol, 4.70 equiv) in tetrahydrofuran (5 mL) were added 2-{1-[(4-bromo-2-nitrophenyl)amino]-3-azabicyclo[3.2.1]octan-3-yl}ethanol (235 mg, 0.63 mmol, 1 equiv) and diisopropyl azodicarboxylate (451 mg, 2.23 mmol, 3.5 equiv) at 0 °C under nitrogen atmosphere. The mixture was stirred at 0 °C for 2 h, treated with water (50 mL), and extracted with ethyl acetate (3×50 mL). The combined organic layers were washed with brine (3×50 mL), dried over anhydrous Na₂SO₄, and concentrated under reduced pressure. The residue was purified by C18 column using water and acetonitrile as mobile phase. Fractions containing the desired compound were evaporated to give 260 mg of methyl 5-[5-(2-{1-[(4-bromo-2-nitrophenyl)amino]-3-azabicyclo[3.2.1]octan-3-yl}ethoxy)-1-methylpyrazol-4-yl]-1-methyl-6-oxopyridine-3-carboxylate (INT-A60-5) as an orange oil (66%). LCMS: m/z (EST), [M+H]⁺=615.15. ¹H NMR (DMSO-d₆, 400 MHz) δ 1.47-1.63 (2H, m), 1.66-1.70 (1H, m), 1.72-1.85 (1H, m), 1.88-1.94 (1H, m), 2.04-2.23 (4H, m), 2.66-2.76 (2H, m), 2.80-2.89 (1H, m), 3.07-3.11 (1H, m), 3.58 (3H, s), 3.73 (3H, s), 3.78 (3H, s), 4.10 (1H, s), 4.11 (1H, s), 6.94 (1H, d), 7.56-7.66 (1H, m), 8.02 (1H, s), 8.12 (1H, d), 8.17 (1H, d), 8.26 (1H, s), 8.43 (1H, d).

Step 6. Preparation of methyl 5-(5-(2-(1-((2-amino-4-bromophenyl)amino)-3-azabicyclo[3.2.1]octan-3-yl)ethoxy)-1-methyl-1H-pyrazol-4-yl)-1-methyl-6-oxo-1,6-dihydropyridine-3-carboxylate (INT-A60-6). A mixture of methyl 5-[5-(2-{1-[(4-bromo-2-nitrophenyl)amino]-3-azabicyclo[3.2.1]octan-3-yl}ethoxy)-1-methylpyrazol-4-yl]-1-methyl-6-oxopyridine-3-carboxylate (240 mg, 0.39 mmol, 1.00 equiv), NH₂NH₂H₂O (39 mg, 0.78 mmol, 2 equiv), and Raney-Ni (33 mg, 0.39 mmol, 1 equiv) in methanol (2 mL) was stirred for 2 h at 0 °C under nitrogen atmosphere. The mixture was filtered. The filter cake was washed with methanol (2×50 mL). The resulting solution was concentrated under reduced pressure. The residue was loaded onto a silica gel column and eluted with dichloromethane/methanol (10/1) to give 200 mg of methyl 5-(5-(2-(1-((2-amino-4-bromophenyl)amino)-3-azabicyclo[3.2.1]octan-3-yl)ethoxy)-1-methyl-1H-pyrazol-4-yl)-1-methyl-6-oxo-1,6-dihydropyridine-3-carboxylate (INT-A60-6) as a yellow solid (84%). LCMS: m/z (EST), [M+H]⁺=585.10. ¹H NMR (DMSO-d₆, 400 MHz) δ 1.43-1.51 (4H, m), 1.63-1.75 (1H, m), 1.77-1.81 (1H, m), 1.87-1.91 (1H, m), 1.95-1.99 (1H, m), 2.01-2.05 (1H, m), 2.09-2.13 (1H, m), 2.67-2.77 (3H, m), 3.03-3.09 (1H, m), 3.58 (3H, s), 3.71 (3H, s), 3.79 (3H, s), 3.97-4.14 (2H, m), 4.87 (2H, s), 6.38 (1H, d), 6.46-6.56 (1H, m), 6.66 (1H, d), 8.00 (1H, s), 8.13 (1H, d), 8.45 (1H, d).

Step 7. Preparation of methyl 5-(5-{2-[1-(2-amino-5-bromo-1,3-benzodiazol-1-yl)-3-azabicyclo[3.2.1]octan-3-yl]ethoxy}-1-methylpyrazol-4-yl)-1-methyl-6-oxopyridine-3-carboxylate (INT-A60-7). A mixture of methyl 5-(5-{2-[1-(2-amino-5-bromo-1,3-benzodiazol-1-yl)-3-azabicyclo[3.2.1]octan-3-yl]ethoxy}-1-methylpyrazol-4-yl)-1-methyl-6-oxopyridine-3-carboxylate (200 mg, 0.32 mmol, 1 equiv) and BrCN (38 mg, 0.36 mmol, 1.1 equiv) in ethanol (2 mL) was stirred for 2 h at 60 °C under nitrogen atmosphere. The mixture was cooled to room temperature, treated with water (50 mL), and extracted with dichloromethane (2×50 mL). The combined organic layers were washed with brine (3×30 mL), dried over anhydrous Na₂SO₄, and concentrated under reduced pressure. The residue was purified by Prep-TLC eluted with (dichloromethane/methanol 12/1) to give 150 mg of methyl 5-(5-{2-[1-(2-amino-5-bromo-1,3-benzodiazol-1-yl)-3-azabicyclo[3.2.1]octan-3-yl]ethoxy}-1-methylpyrazol-4-yl)-1-methyl-6-oxopyridine-3-carboxylate (INT-A60-7) as a brown solid (75%). LCMS: m/z (ESI), [M+H]⁺=611.90. ¹H NMR (DMSO-d₆, 400 MHz) δ 1.04-1.07 (1H, m), 1.22-1.26 (1H, m), 1.48-1.53 (1H, m), 1.71-1.84 (2H, m), 2.20-2.24 (2H, m), 2.27-2.33 (2H, m), 2.62-2.76 (2H, m), 2.76-2.84 (1H, m), 3.02-3.08 (1H, m), 3.58 (3H, s), 3.73 (3H, s), 3.77 (3H, s), 4.09-4.13 (2H, m), 6.08 (2H, s), 6.93-6.97 (1H, m), 7.19 (1H, d), 7.25 (1H, d), 8.01 (1H, s), 8.11 (1H, d), 8.42 (1H, d).

Step 8. Preparation of 5-(5-{2-[1-(2-amino-5-bromo-1,3-benzodiazol-1-yl)-3-azabicyclo[3.2.1]octan-3-yl]ethoxy}-1-methylpyrazol-4-yl)-1-methyl-6-oxopyridine-3-carboxylic acid (INT-A60-8). A mixture of methyl methyl 5-(5-{2-[1-(2-amino-5-bromo-1,3-benzodiazol-1-yl)-3-azabicyclo[3.2.1]octan-3-yl]ethoxy}-1-methylpyrazol-4-yl)-1-methyl-6-oxopyridine-3-carboxylate (140 mg, 0.22 mmol, 1 equiv) and LiOH·H₂O (48 mg, 1.14 mmol, 5 equiv) in tetrahydrofuran/H₂O (2 mL/0.5 mL) was stirred for 2 h at 60 °C under nitrogen atmosphere. The mixture was cooled to room temperature and concentrated. The residue was purified by C18 column using water and acetonitrile as mobile phase. Fractions containing the desired compound were evaporated to dryness to afford 100 mg of 5-(5-{2-[1-(2-amino-5-bromo-1,3-benzodiazol-1-yl)-3-azabicyclo[3.2.1]octan-3-yl]ethoxy}-1-methylpyrazol-4-yl)-1-methyl-6-oxopyridine-3-carboxylic acid (INT-A60-8) as a white solid (73%). LCMS: m/z (ESI), [M+H]⁺=595.95. ¹H NMR (DMSO-d₆, 400 MHz) δ 1.54 (1H, m), 1.73-1.79 (2H, m), 2.15-2.21 (1H, m), 2.28-2.34 (2H, m), 2.63-2.69 (3H, m), 2.72-2.78 (1H, m), 2.85-2.91 (2H, m), 3.05-3.11 (1H, m), 3.49 (3H, s), 3.68 (3H, s), 4.01-4.19 (2H, m), 6.29 (2H, s), 6.90-6.99 (1H, m), 7.22 (1H, d), 7.27 (1H, d), 7.95 (2H, d), 8.24 (1H, d).

Step 9. Preparation of 6-bromo-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (EXAMPLE A59). A mixture of 5-(5-{2-[1-(2-amino-5-bromo-1,3-benzodiazol-1-yl)-3-azabicyclo[3.2.1]octan-3-yl]ethoxy}-1-methylpyrazol-4-yl)-1-methyl-6-oxopyridine-3-carboxylic acid (90 mg, 0.15 mmol, 1 equiv), N,N-diisopropylethylamine (58 mg, 0.45 mmol, 3 equiv), and N,N,N,N-tetramethyl-O-(7-azabenzotriazol-1-yl)uronium hexafluorophospate (114 mg, 0.30 mmol, 2 equiv) in 1,4-dioxane (2 mL) was stirred for 30 min at 60 °C under nitrogen atmosphere. The mixture was cooled to room temperature. The reaction was quenched with water (30 mL) and the mixture was extracted with dichloromethane (2×30 mL). The combined organic layers were washed with brine (3×30 mL), dried over anhydrous Na₂SO₄, and concentrated under reduced pressure. The residue was purified by Prep-TLC eluted with dichloromethane/methanol (12/1) to give 80 mg of 6-bromo-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (EXAMPLE A59) as a white solid (91%). LCMS: m/z (EST), [M+H]⁺=580.20. ¹H NMR (DMSO-d₆, 400 MHz) δ 1.53-1.77 (3H, m), 2.00 (1H, d), 2.39-2.63 (3H, m), 2.64-2.77 (2H, m), 3.16-3.28 (1H, m), 3.29-3.39 (2H, m), 3.62 (3H, s), 3.63-3.69 (1H, m), 3.72 (3H, s), 4.45-4.70 (2H, m), 7.28 (1H, d), 7.72 (1H, d), 8.02 (1H, s), 8.39-8.43 (2H, m), 8.78 (1H, s), 12.81 (1H, s).

Step 10. Preparation of 15,21-dimethyl-6-(4-methylpiperazin-1-yl)-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (A60). To a stirred mixture of 6-bromo-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (75 mg, 0.13 mmol, 1 equiv), 1-methylpiperazine (38 mg, 0.39 mmol, 3 equiv), and BrettPhos Pd G₃ (35 mg, 0.03 mmol, 0.3 equiv) in 1,4-dioxane (3 mL) was added LiHMDS (1.3 mL, 1.30 mmol, 10 equiv) at room temperature. The resulting mixture was stirred for 2 h at 60 °C under nitrogen atmosphere and cooled to room temperature. The reaction was quenched with sat. NH₄Cl (aq.) solution (50 mL) and the mixture was extracted with dichloromethane (2×50 mL). The combined organic layers were washed with brine (3×50 mL), dried over anhydrous Na₂SO₄, and concentrated under reduced pressure. The residue was purified by Prep-TLC eluted with dichloromethane/methanol (10/1) to give 22.3 mg of 15,21-dimethyl-6-(4-methylpiperazin-1-yl)-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (A60) as a yellow solid (28%). LCMS: m/z (ESI), [M+H]⁺=598.35. ¹H NMR (DMSO-d₆, 400 MHz) δ 1.50-1.80 (3H, m), 1.97 (1H, d), 2.26-2.58 (10H, m), 2.55-2.80 (3H, m), 3.03-3.43 (6H, m), 3.63 (3H, s), 3.64-3.70 (1H, m), 3.72 (3H, s), 4.57 (1H, t), 4.69 (1H, d), 6.86 (1H, dd), 7.17 (1H, s), 7.57 (1H, d), 8.29 (1H, d), 8.42 (1H, s), 8.83 (1H, d), 12.56 (1H, s).

Step 11. Preparation of 15,21-dimethyl-6-(4-methylpiperazin-1-yl)-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, isomer 1 (EXAMPLE A60A) and isomer 2 (EXAMPLE A60B). Racemic mixture of 15,21-dimethyl-6-(4-methylpiperazin-1-yl)-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (16.9 mg, 0.02 mmol) was separated by Pre-SFC using CHIRAL ART Cellulose-SB column (2*25 cm, 5 μm) using tert-butyl methyl ether containing 0.10% triethylamine as mobile phase A and ethanol/dichloromethane (1:1) as mobile phase B to give 1 mg of isomer 1 (5%) and 1 mg of isomer 2 (5%) as a yellow solid.

Isomer 1: LCMS: m/z (ESI), [M+H]⁺=598.40. ¹H NMR (DMSO-d₆, 400 MHz) δ 1.50-1.80 (3H, m), 1.97 (1H, d), 2.26-2.58 (10H, m), 2.55-2.80 (3H, m), 3.03-3.43 (6H, m), 3.63 (3H, s), 3.64-3.70 (1H, m), 3.72 (3H, s), 4.55 (1H, t), 4.69 (1H, d), 6.84 (1H, dd), 7.16 (1H, s), 7.55 (1H, d), 8.28 (1H, d), 8.41 (1H, s), 8.83 (1H, d), 12.54 (1H, s). Chiral-HPLC, Rt=3.374 min

Isomer 2: LCMS: m/z (ESI), [M+H]⁺=598.35. ¹H NMR (DMSO-d₆, 400 MHz) δ 1.50-1.80 (3H, m), 1.97 (1H, d), 2.26-2.58 (10H, m), 2.55-2.80 (3H, m), 3.03-3.43 (6H, m), 3.63 (3H, s), 3.64-3.70 (1H, m), 3.72 (3H, s), 4.55 (1H, t), 4.69 (1H, d), 6.85 (1H, dd), 7.16 (1H, s), 7.56 (1H, d), 8.28 (1H, d), 8.41 (1H, s), 8.83 (1H, d), 12.55 (1H, s). Chiral-HPLC, Rt=3.923 min

### Example A61A and Example A61B

### 15,21-Dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, Isomer 1 (Example A61A) and Isomer (Example A61B)

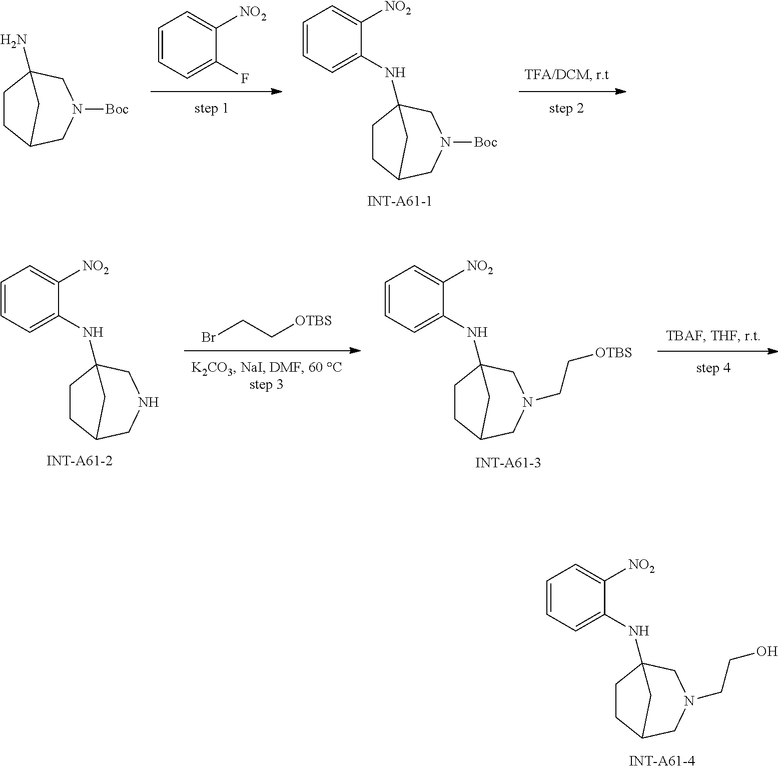

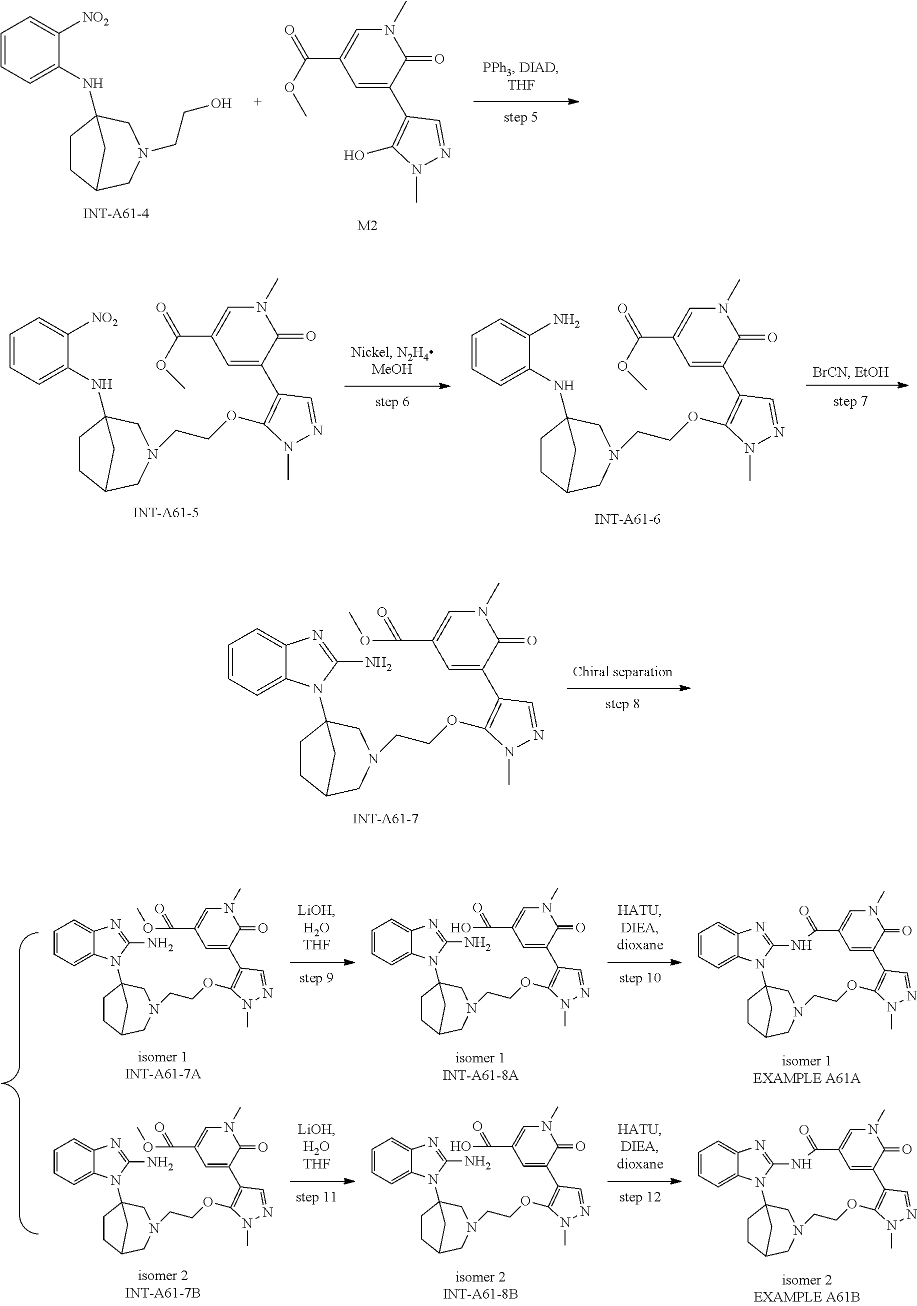

Step 1. Preparation of tert-butyl 1-[(2-nitrophenyl)amino]-3-azabicyclo[3.2.1]octane-3-carboxylate (INT-A61-1). A mixture of tert-butyl 1-amino-3-azabicyclo[3.2.1]octane-3-carboxylate (500 mg, 2.21 mmol, 1 equiv), O-fluoronitrobenzene (312 mg, 2.20 mmol, 1 equiv), and K₂CO₃ (610.66 mg, 4.42 mmol, 2 equiv) in dimethyl sulfoxide (10 mL) was stirred overnight at 120 °C under nitrogen atmosphere and cooled to room temperature. The reaction was quenched with water (50 mL) and the mixture was extracted with ethyl acetate (3×50 mL). The combined organic layers were washed with brine (3×50 mL), dried over anhydrous Na₂SO₄, and concentrated under reduced pressure. The residue was purified by Prep-TLC eluted with petroleum ether/ethyl acetate (5/1) to give 600 mg of tert-butyl 1-[(2-nitrophenyl)amino]-3-azabicyclo[3.2.1]octane-3-carboxylate (INT-A61-1) as an orange oil (78%). LCMS: m/z (ESI), [M+H]⁺=291.95.

Step 2. Preparation of N-(2-nitrophenyl)-3-azabicyclo[3.2.1]octan-1-amine (INT-A61-2). A mixture of tert-butyl 1-[(2-nitrophenyl)amino]-3-azabicyclo[3.2.1]octane-3-carboxylate (550 mg, 1.58 mmol, 1 equiv) in trifluoroacetic acid (2 mL) and dichloromethane (6 mL) was stirred for 2 h at room temperature under nitrogen atmosphere. The mixture was treated with water (50 mL) and saturated NaHCO₃ (aq.). The mixture was extracted with ethyl acetate (3×50 mL). The combined organic layers were washed with brine (3×50 mL), dried over anhydrous Na₂SO₄, and concentrated under reduced pressure to give 380 mg of N-(2-nitrophenyl)-3-azabicyclo[3.2.1]octan-1-amine (INT-A61-2) as an orange oil. This material was used for the next reaction without further purification. LCMS: m/z (ESI), [M+H]⁺=248.00.

Step 3. Preparation of 3-{2-[(tert-butyldimethylsilyl)oxy]ethyl}-N-(2-nitrophenyl)-3azabicyclo[3.2.1]octan-1-amine (INT-A61-3). A mixture of N-(2-nitrophenyl)-3-azabicyclo[3.2.1]octan-1-amine (380 mg, 1.54 mmol, 1 equiv), (2-bromoethoxy)(tert-butyl)dimethylsilane (735 mg, 3.07 mmol, 2 equiv), and K₂CO₃ (425 mg, 3.07 mmol, 2 equiv) in N,N-dimethylformamide (5 mL) was stirred for 2 h at 60 °C under nitrogen atmosphere and cooled to room temperature. The reaction was quenched with water (50 mL) and the mixture was extracted with ethyl acetate (3×50 mL). The combined organic layers were washed with brine (3×50 mL), dried over anhydrous Na₂SO₄, and concentrated under reduced pressure. The residue was purified by Prep-TLC eluted with petroleum ether/ethyl acetate (5/1) to give 450 mg of 3-{2-[(tert-butyldimethylsilyl)oxy]ethyl}-N-(2-nitrophenyl)-3-azabicyclo[3.2.1]octan-1-amine (INT-A61-3) as an orange oil (72%). LCMS: m/z (ESI), [M+H]⁺=406.15.

Step 4. Preparation of 2-{1-[(2-nitrophenyl)amino]-3-azabicyclo[3.2.1]octan-3-yl}ethanol (INT-A61-4). A mixture of 3-{2-[(tert-butyldimethylsilyl)oxy]ethyl}-N-(2-nitrophenyl)-3-azabicyclo[3.2.1]octan-1-amine (400 mg, 1 mmol, 1 equiv) and tetrabutylammonium fluoride (516 mg, 2 mmol, 2 equiv) in tetrahydrofuran (10 mL) was stirred for 2 h at room temperature under nitrogen atmosphere. The reaction was quenched with saturated NaHCO₃ (aq.) (100 mL) and the mixture was extracted with ethyl acetate (3×100 mL). The combined organic layers were washed with brine (3×100 mL), dried over anhydrous Na₂SO₄, and concentrated under reduced pressure to give 280 mg of 2-{1-[(2-nitrophenyl)amino]-3-azabicyclo[3.2.1]octan-3-yl}ethanol (INT-A61-4) as an orange oil. This material was used for the next reaction without further purification. LCMS: m/z (ESI), [M+H]⁺=292.00.

Step 5. Preparation of methyl 1-methyl-5-[1-methyl-5-(2-{1-[(2-nitrophenyl)amino]-3-azabicyclo[3.2.1]octan-3-yl}ethoxy)pyrazol-4-yl]-6-oxopyridine-3-carboxylate (INT-A61-5). To a stirred mixture of 2-{1-[(2-nitrophenyl)amino]-3-azabicyclo[3.2.1]octan-3-yl}ethanol (330 mg, 1.13 mmol, 1 equiv), diisopropyl azodicarboxylate (687 mg, 3.40 mmol, 3 equiv), and triphenylphosphine (891 mg, 3.40 mmol, 3 equiv) in tetrahydrofuran (10 mL) was added methyl 5-(5-hydroxy-1-methylpyrazol-4-yl)-1-methyl-6-oxopyridine-3-carboxylate (M2, 298 mg, 1.13 mmol, 1 equiv) at 0 °C under nitrogen atmosphere. After stirring for 2 h at 0 °C under nitrogen atmosphere, the reaction was quenched with water (100 mL) and the mixture was extracted with dichloromethane (3×100 mL). The combined organic layers were washed with brine (3×100 mL), dried over anhydrous Na₂SO₄, and concentrated under reduced pressure. The residue was purified by Prep-TLC eluted with petroleum ether/ethyl acetate (1/1) to give 400 mg of methyl 1-methyl-5-[1-methyl-5-(2-{l-[(2-nitrophenyl)amino]-3-azabicyclo[3.2.1]octan-3-yl}ethoxy)pyrazol-4-yl]-6-oxopyridine-3-carboxylate (INT-A61-5) as an orange solid (66%).

Step 6. Preparation of methyl 5-[5-(2-{1-[(2-aminophenyl)amino]-3-azabicyclo[3.2.1]octan-3-yl}ethoxy)-1-methylpyrazol-4-yl]-1-methyl-6-oxopyridine-3-carboxylate (INT-A61-6). To a stirred mixture of methyl 1-methyl-5-[1-methyl-5-(2-{1-[(2-nitrophenyl)amino]-3-azabicyclo[3.2.1]octan-3-yl}ethoxy)pyrazol-4-yl]-6-oxopyridine-3-carboxylate (340 mg, 0.634 mmol, 1 equiv), and Raney-Ni (54.29 mg, 0.634 mmol, 1.0 equiv) in methanol (20 mL) was added NH₂NH₂—H₂O (63.44 mg, 1.268 mmol, 2.0 equiv) at 0 °C. The mixture was stirred for 1 h at room temperature under nitrogen atmosphere and filtered. The filter cake was washed with methanol (3×10 mL). The resulting solution was concentrated under reduced pressure. The residue was purified by Prep-TLC eluted with dichloromethane/methanol (12/1) to give 240 mg of methyl 5-[5-(2-{1-[(2-aminophenyl)amino]-3-azabicyclo[3.2.1]octan-3-yl}ethoxy)-1-methylpyrazol-4-yl]-1-methyl-6-oxopyridine-3-carboxylate (INT-A61-6) as a yellow solid (63%). LCMS: m/z (ESI), [M+H]⁺=507.25.

Step 7. Preparation of methyl 5-(5-{2-[1-(2-amino-1,3-benzodiazol-1-yl)-3-azabicyclo[3.2.1]octan-3-yl]ethoxy}-1-methylpyrazol-4-yl)-1-methyl-6-oxopyridine-3-carboxylate (INT-A61-7). A mixture of methyl 5-[5-(2-{1-[(2-aminophenyl)amino]-3-azabicyclo[3.2.1]octan-3-yl}ethoxy)-1-methylpyrazol-4-yl]-1-methyl-6-oxopyridine-3-carboxylate (300 mg, 0.59 mmol, 1 equiv) and BrCN (94 mg, 0.89 mmol, 1.5 equiv) in ethanol (5 mL) was stirred overnight at room temperature under nitrogen atmosphere. The mixture was concentrated under reduced pressure, and the residue was purified by Prep-TLC eluted with dichloromethane/methanol (12/1) to give 200 mg of methyl 5-(5-{2-[1-(2-amino-1,3-benzodiazol-1-yl)-3-azabicyclo[3.2.1]octan-3-yl]ethoxy}-1-methylpyrazol-4-yl)-1-methyl-6-oxopyridine-3-carboxylate (INT-A61-7) as a brown yellow solid (64%). LCMS: m/z (EST), [M+H]⁺=532.25.

Step 8. Preparation of methyl 5-(5-(2-(-1-(2-amino-1H-benzo[d]imidazol-1-yl)-3-azabicyclo[3.2.1]octan-3-yl)ethoxy)-1-methyl-1H-pyrazol-4-yl)-1-methyl-6-oxo-1,6-dihydropyridine-3-carboxylate, isomer 1 (INT-A61-7A) and isomer 2 (INT-A61-7B). Racemic mixture of methyl 5-(5-{2-[1-(2-amino-1,3-benzodiazol-1-yl)-3-azabicyclo[3.2.1]octan-3-yl]ethoxy}-1-methylpyrazol-4-yl)-1-methyl-6-oxopyridine-3-carboxylate (200 mg) was separated using SFC DZ-CHIRALPAK IC-3 column (4.6*50 mm, 3.0 μm), tert-butyl methyl ether containing 0.2% diethylamine as mobile phase A, and ethanol/dichloromethane (1/1) as mobile phase B to give 70 mg of isomer 1 (35%) and 70 mg of isomer 2 (35%) as a yellow solid (35%).

Step 9. Preparation of 5-(5-{2-[1-(2-amino-1,3-benzodiazol-1-yl)-3-azabicyclo[3.2.1]octan-3-yl]ethoxy}-1-methylpyrazol-4-yl)-1-methyl-6-oxopyridine-3-carboxylic acid (INT-A61-8A). A solution of methyl 5-(5-{2-[1-(2-amino-1,3-benzodiazol-1-yl)-3-azabicyclo[3.2.1]octan-3-yl]ethoxy}-1-methylpyrazol-4-yl)-1-methyl-6-oxopyridine-3-carboxylate (INT-A61-7A, 70 mg, 0.13 mmol, 1 equiv) and LiOH—H₂O (22 mg, 0.53 mmol, 4 equiv) in water (1 mL) and tetrahydrofuran (4 mL) was stirred overnight at room temperature under nitrogen atmosphere. The mixture was concentrated under reduced pressure. The residue was purified by prep-HPLC on XBridge Shield RP18 OBD column using water containing 10 mmol/L NH₄HCO₃ and 0.1% NH₃—H₂O and acetonitrile as mobile phase. Fractions containing the desired compound were evaporated to dryness to give 45 mg of 5-(5-{2-[1-(2-amino-1,3-benzodiazol-1-yl)-3-azabicyclo[3.2.1]octan-3-yl]ethoxy}-1-methylpyrazol-4-yl)-1-methyl-6-oxopyridine-3-carboxylic acid (INT-A61-8A) as a yellow solid (70%). LCMS: m/z (ESI), [M+H]⁺=518.10.

Step 10. Preparation of 5,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, isomer1 (EXAMPLE A61A). A mixture of 5-(5-{2-[1-(2-amino-1,3-benzodiazol-1-yl)-3-azabicyclo[3.2.1]octan-3-yl]ethoxy}-1-methylpyrazol-4-yl)-1-methyl-6-oxopyridine-3-carboxylic acid (45 mg, 0.087 mmol, 1 equiv), N,N-Diisopropylethylamineand (33.9, 0.261 mmol, 3 equiv), and N,N,N,N-tetramethyl-O-(7-azabenzotriazol-1-yl)uronium hexafluorophospate (66 mg, 0.17 mmol, 2 equiv) in dioxane (2 mL) was stirred overnight at room temperature under nitrogen atmosphere. After the mixture was concentrated under reduced pressure, the residue was purified by Prep-TLC eluted with dichloromethane/methanol (12/1) to give 26.8 mg of 15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, isomer1 (EXAMPLE A61A) as an off-white solid (61%). LCMS: m/z (ESI), [M+H]⁺=500.15. ¹H NMR (DMSO-d₆, 400 MHz) δ 1.53-1.77 (3H, m), 2.00 (1H, d), 2.39-2.63 (3H, m), 2.64-2.77 (2H, m), 3.16-3.28 (1H, m), 3.29-3.39 (2H, m), 3.62 (3H, s), 3.63-3.69 (1H, m), 3.72 (3H, s), 4.55 (1H, t), 4.68 (1H, d), 7.12-7.23 (2H, m), 7.55-7.60 (1H, m), 7.69-7.75 (1H, m), 8.29 (1H, d), 8.41 (1H, d), 8.83 (1H, d), 12.76 (1H, s).

Step 11. Preparation of 5-(5-{2-[1-(2-amino-1,3-benzodiazol-1-yl)-3-azabicyclo[3.2.1]octan-3-yl]ethoxy}-1-methylpyrazol-4-yl)-1-methyl-6-oxopyridine-3-carboxylic acid (INT-A61-8B). A mixture of methyl 5-(5-{2-[1-(2-amino-1,3-benzodiazol-1-yl)-3-azabicyclo[3.2.1]octan-3-yl]ethoxy}-1-methylpyrazol-4-yl)-1-methyl-6-oxopyridine-3-carboxylate (INT-A61-7B, 70 mg, 0.13 mmol, 1 equiv) and LiOH H₂O (22 mg, 0.53 mmol, 4 equiv) in water (1 mL) and tetrahydrofuran (4 mL) was stirred overnight at room temperature under nitrogen atmosphere. After the mixture was concentrated under reduced pressure, the residue was purified by prep-HPLC with XBridge Shield RP18 OBD column using water containing 10 mmol/L NH₄HCO₃ and 0.1% NH₃·H₂O and acetonitrile as mobile phase. Fractions containing the desired compound were evaporated to dryness to give 45 mg of 5-(5-{2-[-1-(2-amino-1,3-benzodiazol-1-yl)-3-azabicyclo[3.2.1]octan-3-yl]ethoxy}-1-methylpyrazol-4-yl)-1-methyl-6-oxopyridine-3-carboxylic acid (INT-A61-8B) as an off-white solid (70%). LCMS: m/z (ESI), [M+H]⁺=518.15.

Step 12. Preparation of 15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (isomer2 EXAMPLE A61B). A mixture of 5-(5-{2-[1-(2-amino-1,3-benzodiazol-1-yl)-3-azabicyclo[3.2.1]octan-3-yl]ethoxy}-1-methylpyrazol-4-yl)-1-methyl-6-oxopyridine-3-carboxylic acid (45 mg, 0.087 mmol, 1 equiv), N,N-Diisopropylethylamine and (33.9, 0.261 mmol, 3 equiv), and N,N,N,N-tetramethyl-O-(7-azabenzotriazol-1-yl)uronium hexafluorophospate (66 mg, 0.17 mmol, 2 equiv) in dioxane (2 mL) was stirred overnight at room temperature under nitrogen atmosphere. After the mixture was concentrated under reduced pressure, the residue was purified by Prep-TLC eluted with dichloromethane/methanol (12/1) to give 25.8 mg of 15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, isomer 2 (EXAMPLE A61B) as an off-white solid (57%). LCMS: m/z (ESI), [M+H]⁺=500.15. ¹H NMR (DMSO-d₆, 400 MHz) δ 1.53-1.77 (3H, m), 2.00 (1H, d), 2.39-2.63 (3H, m), 2.64-2.77 (2H, m), 3.16-3.28 (1H, m), 3.29-3.39 (2H, m), 3.62 (3H, s), 3.63-3.69 (1H, m), 3.72 (3H, s), 4.55 (1H, t), 4.68 (1H, d), 7.12-7.23 (2H, m), 7.55-7.60 (1H, m), 7.69-7.75 (1H, m), 8.29 (1H, d), 8.41 (1H, d), 8.83 (1H, d), 12.76 (1H, s).

### Example A62A

### 15,21-Dimethyl-5-(morpholin-4-yl)-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, Isomer 1 (Example A62A)

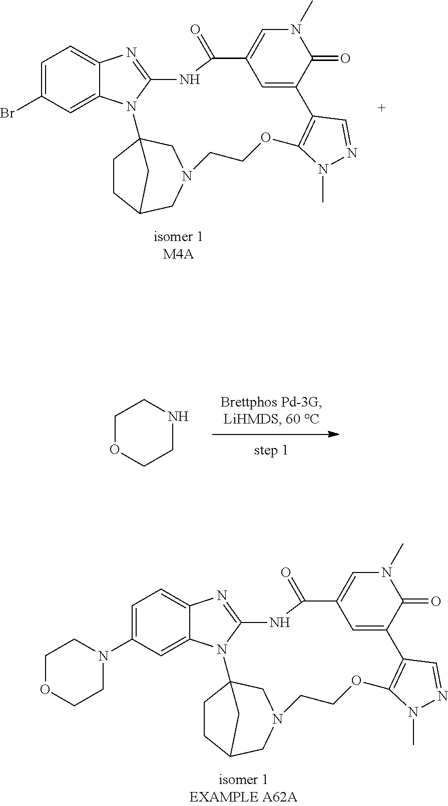 [SMILES: Cn1ncc2c1OCCN1CC3CCC(C3)(C1)n1c(nc3ccc(Br)cc31)NC(=O)c1cc-2c(=O)n(C)c1 ; C1COCCN1 ; Cn1ncc2c1OCCN1CC3CCC(C3)(C1)n1c(nc3ccc(N4CCOCC4)cc31)NC(=O)c1cc-2c(=O)n(C)c1]

Step 1. Preparation of 15,21-dimethyl-5-(morpholin-4-yl)-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, isomer 1 (EXAMPLE A62A). To a stirred mixture of 5-bromo-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (M4A, 50 mg, 0.09 mmol, 1 equiv), morpholine (30 mg, 0.34 mmol, 4 equiv), and BrettPhos Pd G₃ (23 mg, 0.03 mmol, 0.3 equiv) in dioxane (10 mL) was added LiHMDS (0.34 mL, 0.34 mmol, 4 equiv) dropwise at room temperature under nitrogen atmosphere. The reaction mixture was stirred for 30 min at 60 °C. After being cooled to room temperature, the reaction was quenched with sat. NH₄Cl solution (30 mL) and the mixture was extracted with dichloromethane (3×20 mL). The combined organic layers were washed with brine (3×30 mL), dried over anhydrous Na₂SO₄, and concentrated under reduced pressure. The residue was purified by Prep-TLC eluted with dichloromethane/methanol (20/1) to give 22 mg of 15,21-dimethyl-5-(morpholin-4-yl)-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, isomer 1 (EXAMPLE A62A) as a white solid (43%). LCMS: m/z (EST), [M+H]⁺=585.35. ¹H NMR (DMSO-d₆, 400 MHz) δ 1.53-1.82 (3H, m), 2.02 (1H, d), 2.39-2.61 (3H, m), 2.65-2.77 (3H, m), 3.10 (4H, q), 3.17-3.43 (2H, m), 3.62 (3H, s), 3.63-3.69 (1H, m), 3.72 (3H, s), 3.77 (4H, t), 4.54 (1H, t), 4.69 (1H, d), 6.91 (1H, dd), 7.20 (1H, s), 7.44 (1H, d), 8.28 (1H, d), 8.42 (1H, s), 8.83 (1H, d), 12.57 (1H, s).

The following examples were prepared from intermediate M4A using the similiar reaction conditions described above for the synthesis of EXAMPLE A62A: EXAMPLE A63A, EXAMPLE A64A, EXAMPLE A65A, EXAMPLE A66A, EXAMPLE A66C, EXAMPLE A67A, EXAMPLE A68A, EXAMPLE A69A, EXAMPLE A70A, EXAMPLE A71A, EXAMPLE A72A, EXAMPLE A73A, EXAMPLE A76A, EXAMPLE A77A, EXAMPLE A78A, EXAMPLE A79A, EXAMPLE A81A, EXAMPLE A82A, EXAMPLE A83A, EXAMPLE A85A, EXAMPLE A86A, EXAMPLE A87A, EXAMPLE A88A, EXAMPLE A89A, EXAMPLE A90A, EXAMPLE A91A, EXAMPLE A92A, EXAMPLE A95A, EXAMPLE A97A, EXAMPLE A98A, EXAMPLE A99A, EXAMPLE A104A, EXAMPLE A105A, EXAMPLE A106A, EXAMPLE A107A, EXAMPLE A108A. Example A109A.

The structures and the data of m/z and ¹H NMR are listed in Table 1.

### Example A62B

### 15,21-Dimethyl-5-(morpholin-4-yl)-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, Isomer 2 (Example A62B)

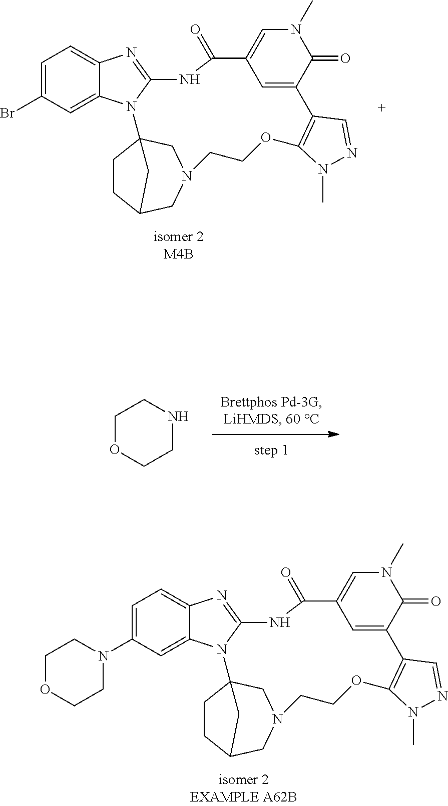 [SMILES: Cn1ncc2c1OCCN1CC3CCC(C3)(C1)n1c(nc3ccc(Br)cc31)NC(=O)c1cc-2c(=O)n(C)c1 ; C1COCCN1 ; Cn1ncc2c1OCCN1CC3CCC(C3)(C1)n1c(nc3ccc(N4CCOCC4)cc31)NC(=O)c1cc-2c(=O)n(C)c1]

Step 1. Preparation of 15,21-dimethyl-5-(morpholin-4-yl)-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, isomer 2 (EXAMPLE A62B). To a stirred mixture of 5-bromo-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22)19-octaene-12,16-dione (M4B, 50 mg, 0.09 mmol, 1 equiv), morpholine (30 mg, 0.34 mmol, 4 equiv), and BrettPhos Pd G₃ (23 mg, 0.03 mmol, 0.3 equiv) in dioxane (5 mL) was added LiHMDS (0.34 mL, 0.34 mmol, 4 equiv) dropwise at room temperature under nitrogen atmosphere. The reaction mixture was stirred for 30 min at 60 °C. After being cooled to room temperature, the reaction was quenched with sat. NH₄Cl solution (30 mL) and the mixture was extracted with dichloromethane (3×20 mL). The combined organic layers were washed with brine (3×30 mL), dried over anhydrous Na₂SO₄, and concentrated under reduced pressure. The residue was purified by Prep-TLC eluted with dichloromethane/methanol (20/1) to give 6.4 mg of 15,21-dimethyl-5-(morpholin-4-yl)-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, isomer 2 (EXAMPLE A62B) as a white solid (12%). LCMS: m/z (EST), [M+H]⁺=585.35. ¹H NMR (DMSO-d₆, 400 MHz) δ 1.53-1.82 (3H, m), 2.02 (1H, d), 2.39-2.61 (3H, m), 2.65-2.77 (3H, m), 3.10 (4H, q), 3.17-3.43 (2H, m), 3.62 (3H, s), 3.63-3.69 (1H, m), 3.72 (3H, s), 3.77 (4H, t), 4.54 (1H, t), 4.69 (1H, d), 6.91 (1H, dd), 7.20 (1H, s), 7.44 (1H, d), 8.28 (1H, d), 8.42 (1H, s), 8.83 (1H, d), 12.57 (1H, s).

The following examples were prepared from intermediate M4B using the similar reaction conditions described above for the synthesis of EXAMPLE A62B: EXAMPLE A63B, EXAMPLE A64B, EXAMPLE A65B, EXAMPLE A66B, EXAMPLE A66D, EXAMPLE A67B, EXAMPLE A68B, EXAMPLE A69B, EXAMPLE A70B, EXAMPLE A71B, EXAMPLE A72B, EXAMPLE A73B, EXAMPLE A76B, EXAMPLE A77B, EXAMPLE A78B, EXAMPLE A79B, EXAMPLE A81B, EXAMPLE A82B, EXAMPLE A83B, EXAMPLE A85B, EXAMPLE A86B, EXAMPLE A87B, EXAMPLE A88B, EXAMPLE A89B, EXAMPLE A90B, EXAMPLE A91B, EXAMPLE A92B, EXAMPLE A95B, EXAMPLE A97B, EXAMPLE A98B, EXAMPLE A99B, EXAMPLE A104B, EXAMPLE A105B, EXAMPLE A106B, EXAMPLE A107B, EXAMPLE A108B. Example A109B.

The structures and the data of m/z and ¹H NMR are listed in Table 1.

### Example A74

### 15,21-Dimethyl-5-(morpholin-4-yl)-23-oxa-2,9,11,15,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,17(34),18(22),19-octaene-12,16-dione

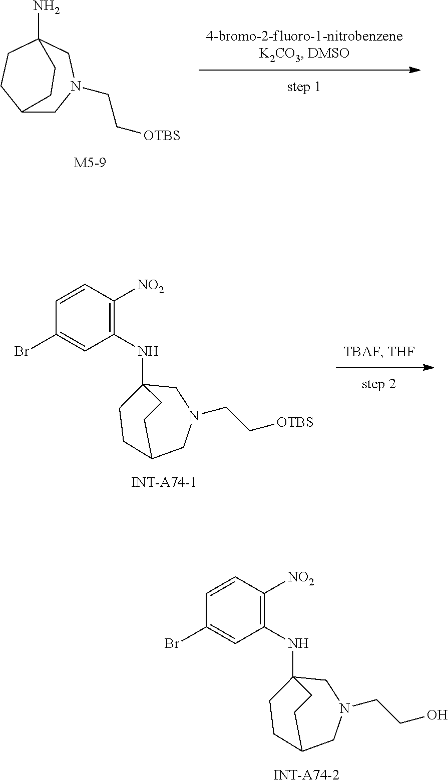 [SMILES: CC(C)(C)[Si](C)(C)OCCN1CC2CCC(N)(CC2)C1 ; CC(C)(C)[Si](C)(C)OCCN1CC2CCC(Nc3cc(Br)ccc3[N+](=O)[O-])(CC2)C1 ; O=[N+]([O-])c1ccc(Br)cc1NC12CCC(CC1)CN(CCO)C2]

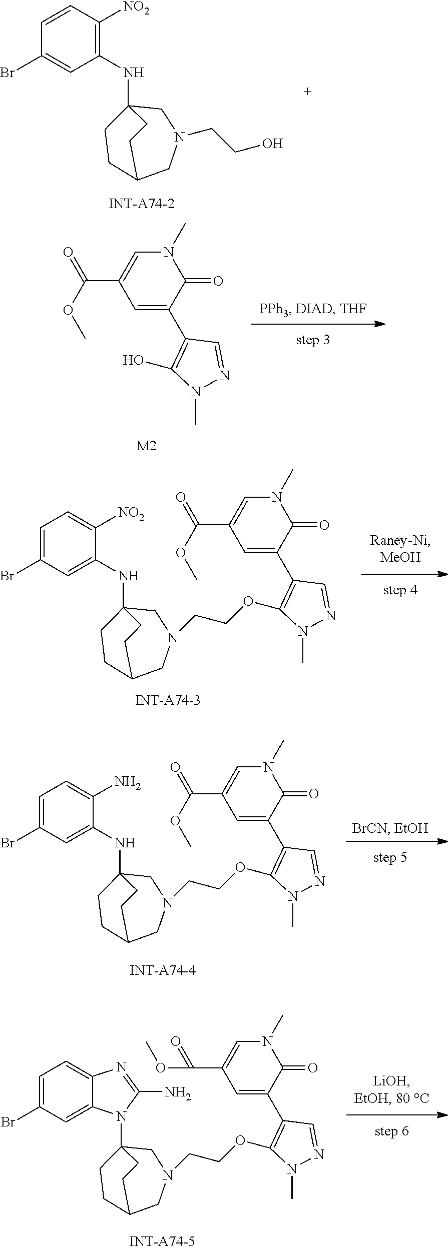 [SMILES: O=[N+]([O-])c1ccc(Br)cc1NC12CCC(CC1)CN(CCO)C2 ; COC(=O)c1cc(-c2cnn(C)c2O)c(=O)n(C)c1 ; "M2" ; COC(=O)c1cc(-c2cnn(C)c2OCCN2CC3CCC(Nc4cc(Br)ccc4[N+](=O)[O-])(CC3)C2)c(=O)n(C)c1 ; COC(=O)c1cc(-c2cnn(C)c2OCCN2CC3CCC(Nc4cc(Br)ccc4N)(CC3)C2)c(=O)n(C)c1 ; COC(=O)c1cc(-c2cnn(C)c2OCCN2CC3CCC(n4c(N)nc5ccc(Br)cc54)(CC3)C2)c(=O)n(C)c1]

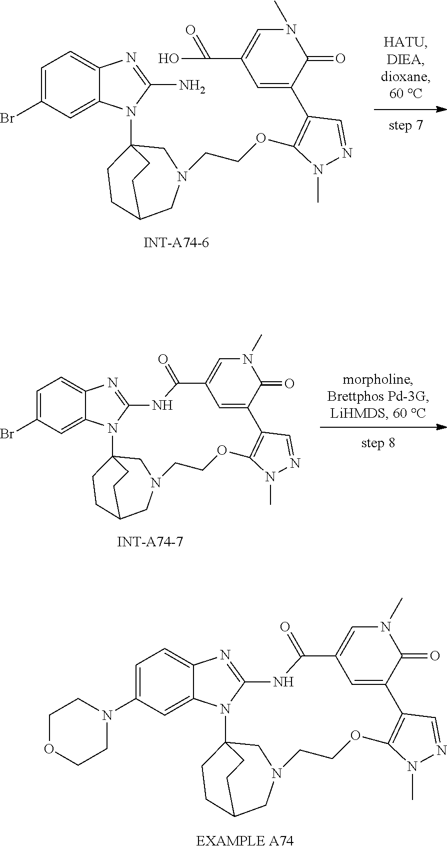 [SMILES: Cn1ncc(-c2cc(C(=O)O)cn(C)c2=O)c1OCCN1CC2CCC(n3c(N)nc4ccc(Br)cc43)(CC2)C1 ; Cn1ncc2c1OCCN1CC3CCC(CC3)(C1)n1c(nc3ccc(Br)cc31)NC(=O)c1cc-2c(=O)n(C)c1 ; Cn1ncc2c1OCCN1CC3CCC(CC3)(C1)n1c(nc3ccc(N4CCOCC4)cc31)NC(=O)c1cc-2c(=O)n(C)c1]

Step 1. Preparation of N-(5-bromo-2-nitrophenyl)-3-{2-[(tert-butyldimethylsilyl)oxy]ethyl}-3-azabicyclo[3.2.2]nonan-1-amine (INT-A74-1). A mixture of 3-{2-[(tert-butyldimethylsilyl)oxy]ethyl}-3-azabicyclo[3.2.2]nonan-1-amine (1 g, 3.35 mmol, 1 equiv), 4-bromo-2-fluoro-1-nitrobenzene (1.84 g, 8.37 mmol, 2.5 equiv), and K₂CO₃ (1.39 g, 10.05 mmol, 3 equiv) in dimethylsulfoxide (10 mL) was stirred for 4 h at 120 °C under nitrogen atmosphere. The mixture was cooled to room temperature. The reaction was quenched with water (50 mL) and the mixture was extracted with ethyl acetate (3×50 mL). The combined organic layers were washed with brine (3×50 mL), dried over anhydrous Na₂SO₄, and concentrated under reduced pressure. The residue was purified by silica gel chromatography eluted with petroleum ether/ethyl acetate (5/1) to give 1.5 g of N-(5-bromo-2-nitrophenyl)-3-{2-[(tert-butyldimethylsilyl)oxy]ethyl}-3-azabicyclo[3.2.2]nonan-1-amine (INT-A74-1) as a yellow oil (85%). LCMS: m/z (ESI), [M+H]+=498.15.

Step 2. Preparation of 2-{1-[(5-bromo-2-nitrophenyl)amino]-3-azabicyclo[3.2.2]nonan-3-yl}ethanol (INT-A74-2). A solution of N-(5-bromo-2-nitrophenyl)-3-{2-[(tert-butyldimethylsilyl)oxy]ethyl}-3-azabicyclo[3.2.2]nonan-1-amine (1.5 g, 3.00 mmol, 1 equiv) and tetrabutylammonium fluoride (1.57 g, 6.01 mmol, 2 equiv) in tetrahydrofuran (15 mL) was stirred for 2 h at room temperature under nitrogen atmosphere. The reaction was quenched with sat. NaHCO₃ (aq.) (100 mL) and the mixture was extracted with dichloromethane (3×100 mL). The combined organic layers were washed with brine (3×100 mL), dried over anhydrous Na₂SO₄, and concentrated under reduced pressure. The residue was purified on silica gel column eluted with dichloromethane/methanol (10/1) to give 1.2 g of 2-{1-[(5-bromo-2-nitrophenyl)amino]-3-azabicyclo[3.2.2]nonan-3-yl}ethanol (INT-A74-2) as yellow oil (93%). LCMS: m/z (ESI), [M+H]⁺=384.00.

Step 3. Preparation of methyl 5-[5-(2-{1-[(5-bromo-2-nitrophenyl)amino]-3-azabicyclo[3.2.2]nonan-3-yl}ethoxy)-1-methylpyrazol-4-yl]-1-methyl-6-oxopyridine-3-carboxylate (INT-A74-3). To a stirred mixture of methyl 5-(5-hydroxy-1-methylpyrazol-4-yl)-1-methyl-6-oxopyridine-3-carboxylate (M2, 0.66 g, 2.49 mmol, 0.8 equiv) and triphenylphosphine (2.46 g, 9.369 mmol, 3 equiv) in tetrahydrofuran (10 mL) were added diisopropylazodicarboxylate (1.89 g, 9.36 mmol, 3 equiv) and 2-{1-[(5-bromo-2-nitrophenyl)amino]-3-azabicyclo[3.2.2]nonan-3-yl}ethanol (1.20 g, 3.11 mmol, 1 equiv) at 0 °C. The mixture was stirred for 2 h at 0 °C under nitrogen atmosphere. The reaction was quenched with water (50 mL) and the mixture was extracted with ethyl acetate (3×50 mL). The combined organic layers were washed with brine (3×50 mL), dried over anhydrous Na₂SO₄, and concentrated under reduced pressure. The residue was purified by Prep-TLC and eluted with petroleum ether/ethyl acetate (1/1) to give 1.2 g of methyl 5-[5-(2-{1-[(5-bromo-2-nitrophenyl)amino]-3-azabicyclo[3.2.2]nonan-3-yl}ethoxy)-1-methylpyrazol-4-yl]-1-methyl-6-oxopyridine-3-carboxylate (INT-A74-3) as a yellow oil (57%). LCMS: m/z (ESI), [M+H]⁺=629.25. ¹H NMR (DMSO-d₆, 400 MHz) δ 1.58-1.76 (9H, m), 2.56-2.79 (2H, m), 2.90 (4H, q), 3.56 (3H, s), 3.72 (6H, d), 4.13 (2H, t), 6.70-6.85 (1H, m), 7.01 (1H, d), 7.95-8.12 (4H, m), 8.28 (1H, d).

Step 4. Preparation of methyl 5-[5-(2-{1-[(2-amino-5-bromophenyl)amino]-3-azabicyclo[3.2.2]nonan-3-yl}ethoxy)-1-methylpyrazol-4-yl]-1-methyl-6-oxopyridine-3-carboxylate (INT-A74-4). To a stirred mixture of Raney Nickel (0.90 g) and methyl 5-[5-(2-{1-[(5-bromo-2-nitrophenyl)amino]-3-azabicyclo[3.2.2]nonan-3-yl}ethoxy)-1-methylpyrazol-4-yl]-1-methyl-6-oxopyridine-3-carboxylate (1.1 g, 1.74 mmol, 1 equiv) in methanol (8 mL) was added NH₂NH₂·H₂O (47 mg, 0.95 mmol, 2 equiv) at 0 °C under nitrogen atmosphere. The mixture was stirred for 1 h at 0 °C. The mixture was filtered and the filter cake was washed with methanol (2×20 mL). The resulting solution was concentrated under reduced pressure. The residue was purified by Prep-TLC and eluted with dichloromethane/methanol (10/1) to give 910 mg of methyl 5-[5-(2-{l-[(2-amino-5-bromophenyl)amino]-3-azabicyclo[3.2.2]nonan-3-yl}ethoxy)-1-methylpyrazol-4-yl]-1-methyl-6-oxopyridine-3-carboxylate (INT-A74-4) as a light yellow solid (76%). LCMS: m/z (ESI), [M+H]⁺=599.20.

Step 5. Preparation of methyl 5-(5-{2-[1-(2-amino-6-bromo-1,3-benzodiazol-1-yl)-3-azabicyclo[3.2.2]nonan-3-yl]ethoxy}-1-methylpyrazol-4-yl)-1-methyl-6-oxopyridine-3-carboxylate (INT-A74-5). A mixture of methyl 5-[5-(2-{1-[(2-amino-5-bromophenyl)amino]-3-azabicyclo[3.2.2]nonan-3-yl}ethoxy)-1-methylpyrazol-4-yl]-1-methyl-6-oxopyridine-3-carboxylate (900 mg, 1.50 mmol, 1 equiv) and BrCN (174 mg, 1.65 mmol, 1.1 equiv) in ethyl alcohol (10 mL) was stirred for 2 h at room temperature under nitrogen atmosphere. The resulting mixture was concentrated under vacuum. The residue was purified by Prep-TLC and eluted with dichloromethane/methanol (12/1) to give 640 mg of methyl 5-(5-{2-[1-(2-amino-6-bromo-1,3-benzodiazol-1-yl)-3-azabicyclo[3.2.2]nonan-3-yl]ethoxy}-1-methylpyrazol-4-yl)-1-methyl-6-oxopyridine-3-carboxylate (INT-A74-5) as a light yellow solid (68%). LCMS: m/z (ESI), [M+H]⁺=624.10. ¹H NMR (DMSO-d₆, 400 MHz) δ 1.24 (1H, s), 1.78-1.95 (8H, m), 2.60-2.74 (2H, m), 2.85-2.95 (4H, m), 3.57 (3H, s), 3.71 (6H, d), 4.08-4.17 (2H, m), 5.95 (2H, s), 7.04 (2H, s), 7.47 (1H, s), 7.96 (1H, s), 8.12 (1H,), 8.39 (1H, d).

Step 6. 5-(5-{2-[1-(2-amino-6-bromo-1,3-benzodiazol-1-yl)-3-azabicyclo[3.2.2]nonan-3-yl]ethoxy}-1-methylpyrazol-4-yl)-1-methyl-6-oxopyridine-3-carboxylic acid (INT-A74-6). A mixture of methyl 5-(5-{2-[l-(2-amino-6-bromo-1,3-benzodiazol-1-yl)-3-azabicyclo[3.2.2]nonan-3-yl]ethoxy}-1-methylpyrazol-4-yl)-1-methyl-6-oxopyridine-3-carboxylate (620 mg, 0.99 mmol, 1 equiv) and LiOH (28 mg, 1.19 mmol, 1.2 equiv) in tetrahydrofuran/H₂O (4 mL/1 mL) was stirred for 2 h at 80 °C under nitrogen atmosphere. The mixture was cooled to room temperature and concentrated under vacuum to give 550 mg of 5-(5-{2-[1-(2-amino-6-bromo-1,3-benzodiazol-1-yl)-3-azabicyclo[3.2.2]nonan-3-yl]ethoxy}-1-methylpyrazol-4-yl)-1-methyl-6-oxopyridine-3-carboxylic acid (INT-A74-6) as a white solid (90%). This material was used for the next reaction without purification.

Step 7. Preparation of 5-bromo-15,21-dimethyl-23-oxa-2,9,11,15,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,17(34),18(22),19-octaene-12,16-dione (INT-A74-7). A mixture of 5-(5-{2-[1-(2-amino-6-bromo-1,3-benzodiazol-1-yl)-3-azabicyclo[3.2.2]nonan-3-yl]ethoxy}-1-methylpyrazol-4-yl)-1-methyl-6-oxopyridine-3-carboxylic acid (550 mg, 0.90 mmol, 1 equiv), N,N,N,N-Tetramethyl-O-(7-azabenzotriazol-1-yl)uronium hexafluorophospate (1027 mg, 2.70 mmol, 3 equiv) and N,N-diisopropylethylamine (174 mg, 1.35 mmol, 1.5 equiv) in dioxane (6 mL) was stirred for 2 h at 60 °C under nitrogen atmosphere. The mixture was cooled to room temperature and concentrated under vacuum. The residue was purified by prep-HPLC with XBridge Prep OBD C18 column using water containing 10 mmol/L NH₄HCO₃ and acetonitrile as mobile phase. Fractions containing the desired compound were evaporated to dryness to give 500 mg of 5-bromo-15,21-dimethyl-23-oxa-2,9,11,15,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,17(34),18(22),19-octaene-12,16-dione (INT-A74-7) as a yellow solid (88%). LCMS: m/z (ESI), [M+H]⁺=592.15.

Step 8. Preparation of 15,21-dimethyl-5-(morpholin-4-yl)-23-oxa-2,9,11,15,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,17(34),18(22),19-octaene-12,16-dione (EXAMPLE A74). To a stirred mixture of 5-bromo-15,21-dimethyl-23-oxa-2,9,11,15,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,17(34),18(22),19-octaene-12,16-dione (130 mg, 0.21 mmol, 1 equiv), morpholine (57 mg, 0.65 mmol, 3 equiv), and BrettPhos Pd G₃ (59 mg, 0.06 mmol, 0.3 equiv) in 1,4-dioxane (12 mL) was added LiHMDS (5.2 mL, 5.20 mmol, 6 equiv) at room temperature under nitrogen atmosphere. The mixture was stirred for 1 h at 60 °C and cooled to room temperature. The reaction was quenched with sat. NH₄Cl (aq.) (15 mL) and the mixture was extracted with CH₂Cl₂ (3×30 mL). The combined organic layers were dried over anhydrous Na₂SO₄ and concentrated under reduced pressure. The residue was purified by Prep-TLC and eluted with methylene dichloride/methanol (12/1) to give 38.3 mg of 15,21-dimethyl-5-(morpholin-4-yl)-23-oxa-2,9,11,15,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,17(34),18(22),19-octaene-12,16-dione (EXAMPLE A74) as a white solid (28%). LCMS: m/z (EST), [M+H]⁺=599.25. ¹H NMR (DMSO-d₆, 400 MHz) δ 1.82-2.02 (4H, m), 2.02-2.12 (3H, m), 2.80 (2H, d), 2.87 (2H, t), 3.08 (4H, t), 3.38-3.52 (4H, m), 3.62 (3H, s), 3.75 (3H, s), 3.77-3.85 (4H, m), 4.28 (2H, t), 6.93 (1H, dd), 7.34 (1H, d), 7.49 (1H, d), 8.31 (2H, d), 9.12 (1H, d), 13.20 (1H, s).

### Example A75

### 15,21-Dimethyl-5-(4-methylpiperazin-1-yl)-23-oxa-2,9,11,15,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,17(34),18(22),19-octaene-12,16-dione

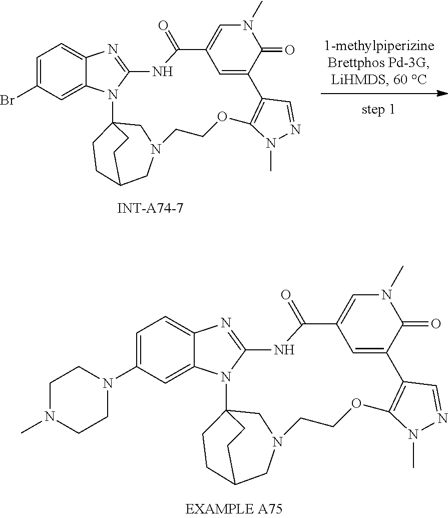 [SMILES: Cn1ncc2c1OCCN1CC3CCC(CC3)(C1)n1c(nc3ccc(Br)cc31)NC(=O)c1cc-2c(=O)n(C)c1 ; CN1CCN(c2ccc3nc4n(c3c2)C23CCC(CC2)CN(CCOc2c(cnn2C)-c2cc(cn(C)c2=O)C(=O)N4)C3)CC1]

Step 1. Preparation of 15,21-dimethyl-5-(4-methylpiperazin-1-yl)-23-oxa-2,9,11,15,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,17(34),18(22),19-octaene-12,16-dione (Example A75). To a stirred mixture of 5-bromo-15,21-dimethyl-23-oxa-2,9,11,15,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,17(34),18(22),19-octaene-12,16-dione (130 mg, 0.21 mmol, 1 equiv), 1-methylpiperazine (65.93 mg, 0.65 mmol, 3 equiv), and BrettPhos Pd G₃ (59.67 mg, 0.06 mmol, 0.3 equiv) in 1,4-dioxane (13 mL) was added LiHMDS (5.2 mL, 5.2 mmol, 6 equiv) at room temperature under nitrogen atmosphere. The mixture was stirred for 1 h at 60 °C and cooled to room temperature. The mixture was treated with sat. NH₄Cl (aq.) (30 mL) and the mixture was extracted with dichloromethane (3×20 mL). The combined organic layers were washed with brine (3×30 mL), dried over anhydrous Na₂SO₄, and concentrated under reduced pressure. The residue was purified by Prep-TLC and eluted with methylene dichloride/methanol (12/1) to give 31 mg of 15,21-dimethyl-5-(4-methylpiperazin-1-yl)-23-oxa-2,9,11,15,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,17(34),18(22),19-octaene-12,16-dione (Example A75) as a light yellow solid (22%). LCMS: m/z (EST), [M+H]⁺=612.30. ¹H NMR (DMSO-d₆, 400 MHz) δ 1.85-1.99 (4H, m), 2.05-2.09 (3H, m), 2.24 (3H, t), 2.46-2.50 (4H, m), 2.80 (2H, d), 2.87 (2H, t), 3.09 (4H, t), 3.52-3.59 (4H, m), 3.62 (3H, s), 3.75 (3H, s), 4.28 (2H, t), 6.92 (1H, dd), 7.33 (1H, d), 7.47 (1H, d), 8.31 (2H, d), 9.12 (1H, d), 13.19 (1H, s).

### Example A80A, Example A80A1, Example A80A2

### 15,21-Dimethyl-5-[3-(4-methylpiperazin-1-yl)pyrrolidin-1-yl]-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, Isomer A (Example A80A), Isomer A1 (Example A80A1), and Isomer A2 (Example A80A2)

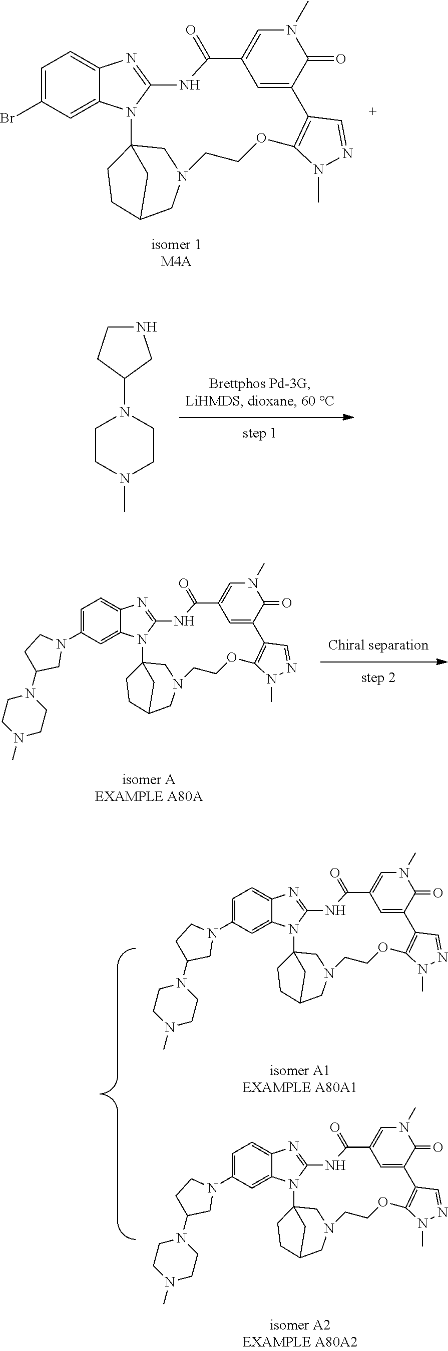 [SMILES: Cn1ncc2c1OCCN1CC3CCC(C3)(C1)n1c(nc3ccc(Br)cc31)NC(=O)c1cc-2c(=O)n(C)c1 ; CN1CCN(C2CCNC2)CC1 ; CN1CCN(C2CCN(c3ccc4nc5n(c4c3)C34CCC(CN(CCOc6c(cnn6C)-c6cc(cn(C)c6=O)C(=O)N5)C3)C4)C2)CC1 ; CN1CCN(C2CCN(c3ccc4nc5n(c4c3)C34CCC(CN(CCOc6c(cnn6C)-c6cc(cn(C)c6=O)C(=O)N5)C3)C4)C2)CC1 ; CN1CCN(C2CCN(c3ccc4nc5n(c4c3)C34CCC(CN(CCOc6c(cnn6C)-c6cc(cn(C)c6=O)C(=O)N5)C3)C4)C2)CC1]

Step 1. Preparation of 15,21-dimethyl-5-[3-(4-methylpiperazin-1-yl)pyrrolidin-1-yl]-23-oxa-2,9,11,15,20,21,26-heptaazaheptacyclo[24.4.1.1\hat{ }{1,28}.1A {13,17}.0\hat{ }{2,10}.0\hat{ }{3,8}.0\hat{ }{18,22}]tritriaconta-3,5,7,9,13,17(33),18(22),19-octaene-12,16-dione, isomer A (EXAMPLE A80A). To a stirred mixture of 5-bromo-15,21-dimethyl-23-oxa-2,9,11,15,20,21,26-heptaazaheptacyclo[24.4.1.1 \hat{ }{1,28}.1\hat{ }{13,17}.0\hat{ }{2,10}.0\hat{ }{3,8}0\hat{ }{18,22}]tritriaconta-3,5,7,9,13,17(33),18(22),19-octaene-12,16-dione (M4A, 90 mg, 0.15 mmol, 1 equiv), 1-methyl-4-(pyrrolidin-3-yl)piperazine (79.01 mg, 0.46 mmol, 3 equiv), and BrettPhos Pd G₃ (42.31 mg, 0.04 mmol, 0.3 equiv) was added LiHMDS (3.6 mL, 3.6 mmol, 24 equiv) dropwise at 0 °C under nitrogen atmosphere. The mixture was stirred for 1 h at 60 °C and then cooled to room temperature. The reaction was quenched with sat. NH₄Cl solution (aq.) (10 mL) and the mixture was extracted with dichloromethane (3×20 mL). The combined organic layers were washed with brine (3×30 mL), dried over anhydrous Na₂SO₄, and concentrated under reduced pressure. The residue was purified by Prep-HPLC with XBridge Shield RP18 OBD column using water containing 10 mmol/L NH₄HCO₃ and 0.1% NH₃·H₂O and acetonitrile as mobile phase. Fractions containing the desired compound were evaporated to dryness to give 2.6 mg of 15,21-dimethyl-5-[3-(4-methylpiperazin-1-yl)pyrrolidin-1-yl]-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (isomer A, EXAMPLE A80A) as a yellow solid (2%). LCMS: m/z (ESI), [M+H]+=667.40. ¹H NMR (DMSO-d₆, 400 MHz) δ 1.57-1.87 (4H, m), 2.00 (1H, s), 2.16 (3H, s), 2.23-2.63 (12H, m), 2.66-2.76 (2H, m), 2.91 (1H, t), 3.07 (1H, q), 3.20-3.59 (6H, m), 3.62 (3H, s), 3.63-3.69 (1H, m), 3.72 (3H, s), 4.57 (1H, t), 4.69 (1H, d), 6.49 (1H, dd), 6.75 (1H, s), 7.38 (1H, d), 8.26 (1H, d), 8.41 (1H, s), 8.81 (1H, d), 12.51 (1H, s). SFC-HPLC, Rt=1.029 min.

Step 2. Preparation of 15,21-dimethyl-5-[3-(4-methylpiperazin-1-yl)pyrrolidin-1-yl]-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, isomer A1 (EXAMPLE A80A1) and isomer A2 (EXAMPLE A80A2). The mixture of 15,21-dimethyl-5-[3-(4-methylpiperazin-1-yl)pyrrolidin-1-yl]-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (EXAMPLE A80A, 20 mg, 0.17 mmol) was separated by Prep-Chiral-HPLC with XBridge Shield RP18 OBD column (30*150 mm, 5 μm) using water containing 10 mmol/L NH₄HCO₃ and 0.1% NH₃·H₂O and acetonitrile as mobile phase to give 8.5 mg of isomer A1 (EXAMPLE A80A1, 18%) and 3.2 mg of isomer A2 (EXAMPLE A80A2, 6%) a yellow solid.

Isomer A1: LCMS: m/z (ESI), [M+H]⁺=667.45. ¹H NMR (DMSO-d₆, 400 MHz) δ 1.57-1.87 (4H, m), 2.00 (1H, s), 2.16 (3H, s), 2.23-2.63 (12H, m), 2.66-2.76 (2H, m), 2.91 (1H, t), 3.07 (1H, q), 3.20-3.59 (6H, m), 3.62 (3H, s), 3.63-3.69 (1H, m), 3.72 (3H, s), 4.57 (1H, t), 4.69 (1H, d), 6.49 (1H, dd), 6.76 (1H, s), 7.38 (1H, d), 8.26 (1H, d), 8.42 (1H, s), 8.82 (1H, d), 12.53 (1H, s). CHIRAL-HPLC, Rt=5.235 min.

Isomer A2: LCMS: m/z (ESI), [M+H]⁺=667.45. ¹H NMR (DMSO-d₆, 400 MHz) δ 1.57-1.87 (4H, m), 2.00 (1H, s), 2.16 (3H, s), 2.23-2.63 (12H, m), 2.66-2.76 (2H, m), 2.91 (1H, t), 3.07 (1H, q), 3.20-3.59 (6H, m), 3.62 (3H, s), 3.63-3.69 (1H, m), 3.72 (3H, s), 4.57 (1H, t), 4.69 (1H, d), 6.47 (1H, dd), 6.76 (1H, s), 7.37 (1H, d), 8.26 (1H, d), 8.42 (1H, s), 8.82 (1H, d), 12.49 (1H, s). CHIRAL-HPLC, Rt=6.080 min

### Example A80B, Example A80B1, Example A80B2

### 15,21-Dimethyl-5-[3-(4-methylpiperazin-1-yl)pyrrolidin-1-yl]-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, Isomer B (Example A80B), Isomer B1 (Example A80B1), and Isomer B2 (Example A80B2)

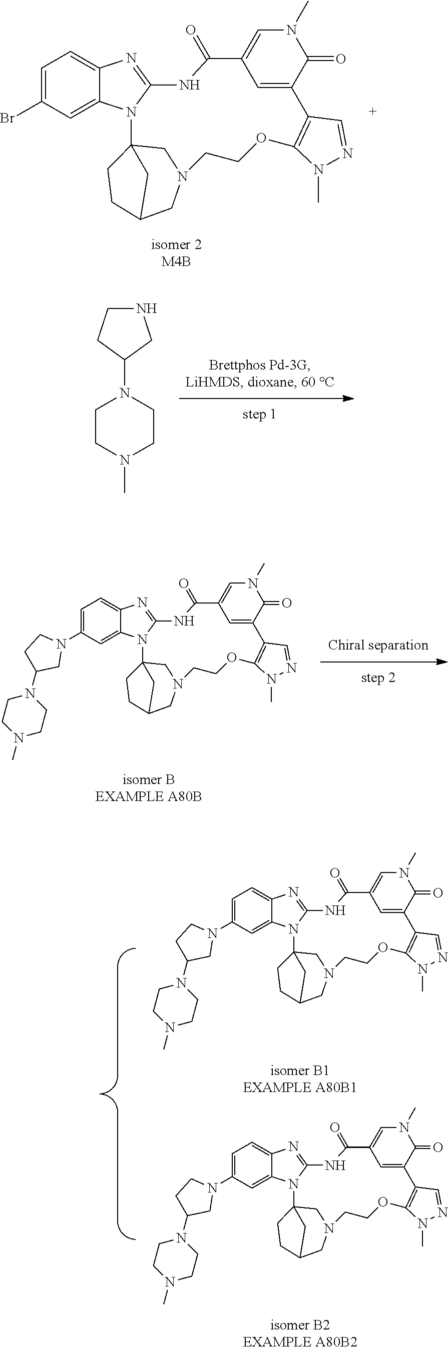 [SMILES: Cn1ncc2c1OCCN1CC3CCC(C3)(C1)n1c(nc3ccc(Br)cc31)NC(=O)c1cc-2c(=O)n(C)c1 ; CN1CCN(C2CCNC2)CC1 ; CN1CCN(C2CCN(c3ccc4nc5n(c4c3)C34CCC(CN(CCOc6c(cnn6C)-c6cc(cn(C)c6=O)C(=O)N5)C3)C4)C2)CC1 ; CN1CCN(C2CCN(c3ccc4nc5n(c4c3)C34CCC(CN(CCOc6c(cnn6C)-c6cc(cn(C)c6=O)C(=O)N5)C3)C4)C2)CC1 ; CN1CCN(C2CCN(c3ccc4nc5n(c4c3)C34CCC(CN(CCOc6c(cnn6C)-c6cc(cn(C)c6=O)C(=O)N5)C3)C4)C2)CC1]

Step 1. Preparation of 15,21-dimethyl-5-[3-(4-methylpiperazin-1-yl)pyrrolidin-1-yl]-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, isomer B (EXAMPLE A801B). To a stirred mixture of 5-bromo-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3, 8}.0\hat{ }{18,22}]tritriaconta-3,5,7,9,13,17(33),18(22),19-octaene-12,16-dione (M4B, 90 mg, 0.15 mmol, 1 equiv), 1-methyl-4-(pyrrolidin-3-yl)piperazine (79.01 mg, 0.46 mmol, 3 equiv), and BrettPhos Pd G₃ (42.31 mg, 0.04 mmol, 0.3 equiv) was added LiHMDS (3.6 mL, 3.60 mmol, 24 equiv) dropwise at 0 °C under nitrogen atmosphere. The mixture was stirred for 1 h at 60 °C and then cooled to room temperature. The reaction was quenched with sat. NH₄Cl solution (aq.) (10 mL) and the mixture was extracted with dichloromethane (3×20 mL). The combined organic layers were washed with brine (3×30 mL), dried over anhydrous Na₂SO₄, and concentrated under reduced pressure. The residue was purified by Prep-HPLC using XBridge Shield RP18 OBD column, water containing 10 mmol/L NH₄HCO₃ and 0.1% NH₃·H₂O, and acetonitrile as mobile phase. Fractions containing the desired compound were evaporated to dryness to give 1.6 mg of 15,21-dimethyl-5-[3-(4-methylpiperazin-1-yl)pyrrolidin-1-yl]-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, isomer B (EXAMPLE A80B) as a yellow solid (1%). LCMS: m/z (EST), [M+H]⁺=667.40 ¹H NMR (DMSO-d₆, 400 MHz) δ 1.57-1.87 (4H, m), 2.00 (1H, s), 2.16 (3H, s), 2.23-2.63 (12H, m), 2.66-2.76 (2H, m), 2.91 (1H, t), 3.07 (1H, q), 3.20-3.59 (6H, m), 3.62 (3H, s), 3.63-3.69 (1H, m), 3.72 (3H, s), 4.57 (1H, t), 4.69 (1H, d), 6.49 (1H, d), 6.75 (1H, s), 7.38 (1H, d), 8.26 (1H, d), 8.41 (1H, s), 8.81 (1H, d), 12.51 (1H, s). SFC-HPLC, Rt=0.869,

Step 2. Preparation of 15,21-dimethyl-5-[3-(4-methylpiperazin-1-yl)pyrrolidin-1-yl]-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, isomer B1(EXAMPLE A80B1) and isomer B2 (EXAMPLE A80B2). The mixture of 15,21-dimethyl-5-[3-(4-methylpiperazin-1-yl)pyrrolidin-1-yl]-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (EXAMPLE A80B, 18 mg, 0.17 mmol) was separated by Prep-Chiral-HPLC with XBridge Prep OBD C18 column (30*150 mm, 5 μm) using water containing 10 mmol/L NH4HCO3 and acetonitrile as mobile phase to give 2.4 mg of isomer B1(EXAMPLE A80B1, 5%) and 1.2 mg of isomer B2 (EXAMPLE A80B2, 2%) as a yellow solid.

Isomer B1: LCMS: m/z (ESI), [M+H]⁺=667. ¹H NMR (DMSO-d₆, 400 MHz) δ 1.57-1.87 (4H, m), 2.00 (1H, s), 2.16 (3H, s), 2.23-2.63 (12H, m), 2.66-2.76 (2H, m), 2.91 (1H, t), 3.07 (1H, q), 3.20-3.59 (6H, m), 3.62 (3H, s), 3.63-3.69 (1H, m), 3.72 (3H, s), 4.57 (1H, t), 4.69 (1H, d), 6.49 (1H, d), 6.75 (1H, s), 7.38 (1H, d), 8.26 (1H, d), 8.41 (1H, s), 8.81 (1H, d), 12.51 (1H, s). CHIRAL-HPLC, Rt=3.906 min.

Isomer B2: LCMS: m/z (ESI), [M+H]⁺=667.45. ¹H NMR (DMSO-d₆, 400 MHz) δ 1.57-1.87 (4H, m), 2.00 (1H, s), 2.16 (3H, s), 2.23-2.63 (12H, m), 2.66-2.76 (2H, m), 2.91 (1H, t), 3.07 (1H, q), 3.20-3.59 (6H, m), 3.62 (3H, s), 3.63-3.69 (1H, m), 3.72 (3H, s), 4.57 (1H, t), 4.69 (1H, d), 6.49 (1H, d), 6.75 (1H, s), 7.38 (1H, d), 8.26 (1H, d), 8.41 (1H, s), 8.81 (1H, d), 12.51 (1H, s). CHIRAL-HPLC, Rt=5.026 min.

### Example A84A and Example A84B

### 15,21-Dimethyl-5-[4-fluoro-3-(N,N-dimethylamino)pyrrolidinyl]-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, Isomer 1 (Example A84A) and Isomer 2 (Example A84B)

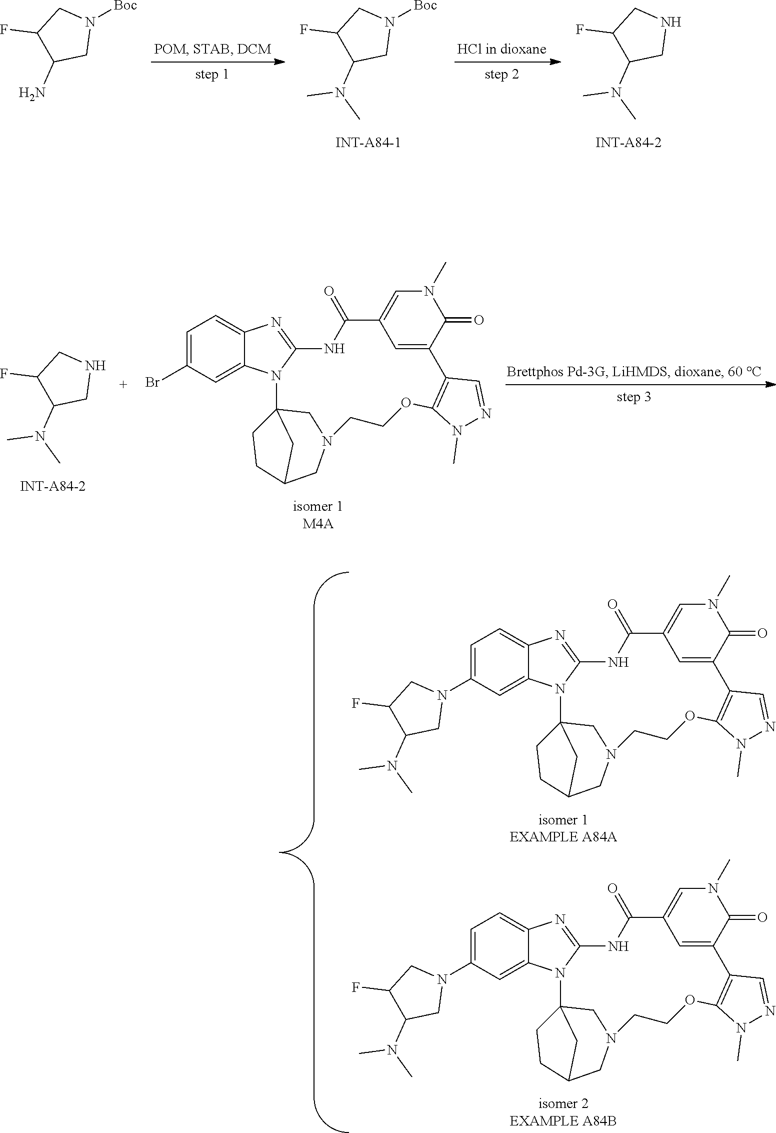

Step 1. Preparation of tert-butyl 3-(dimethylamino)-4-fluoropyrrolidine-1-carboxylate (INT-A84-1). To a stirred mixture of tert-butyl 3-amino-4-fluoropyrrolidine-1-carboxylate (500 mg, 2.44 mmol, 1 equiv), polyoxymethylene (4410.29 mg, 48.96 mmol, 20 equiv), and NaBH(OAc)₃ (2075.35 mg, 9.79 mmol, 4 equiv) in Cl₂Cl₂ (20 mL) were added trimethylamine (990.90 mg, 9.79 mmol, 4 equiv) dropwise at 0 °C under nitrogen atmosphere. The mixture was stirred for 2 h at 0 °C. The reaction was quenched with water (10 mL) and the mixture was extracted with CH₂Cl₂ (3×20 mL). The combined organic layers were washed with brine (3×20 mL), dried over anhydrous Na₂SO₄, and concentrated under reduced pressure. The residue was purified on silica gel column eluted with petroleum CH₂Cl₂/methanol (10/1) to give 500 mg of tert-butyl 3-(dimethylamino)-4-fluoropyrrolidine-1-carboxylate (INT-A84-1) as a brown oil (79%). ¹H NMR (DMSO-d₆, 400 MHz) δ 1.40 (9H, s), 2.10-2.23 (5H, m), 2.85-2.95 (1H, m), 3.25 (1H, d), 3.47-3.64 (4H, m), 5.11-5.23 (1H, m).

Step 2. Preparation of 4-fluoro-N,N-dimethylpyrrolidin-3-amine (INT-A84-2). A mixture of tert-butyl 3-(dimethylamino)-4-fluoropyrrolidine-1-carboxylate (500 mg, 2.152 mmol, 1 equiv) and HCl in 1,4-dioxane (4 mL) was stirred for 2 h at room temperature under nitrogen atmosphere. To this reaction solution was added solid K₂CO₃. After filtration, the filtrate was concentrated under reduced pressure and give 300 mg of 4-fluoro-N,N-dimethylpyrrolidin-3-amine (INT-A84-2) as a colorless oil. This material was used for the next reaction without further purification. ¹H NMR (DMSO-d₆, 400 MHz) δ 2.05-2.25 (6H, m), 2.40-2.65 (2H, m), 2.76-3.48 (4H, m), 4.94-5.08 (1H, m)

Step 3. Preparation of 15,21-dimethyl-5-[4-fluoro-3-(N,N-dimethylamino)pyrrolidinyl]-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, isomer 1 (EXAMPLE A84A) and isomer 2 (EXAMPLE A84B). To a stirred mixture of 5-bromo-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (M4A, 150 mg, 0.25 mmol, 1.00 equiv), BrettPhos Pd G₃ (70.52 mg, 0.07 mmol, 0.3 equiv), and 4-fluoro-N,N-dimethylpyrrolidin-3-amine (102.83 mg, 0.77 mmol, 3 equiv) in 1,4-dioxane (15 mL) was added LiHMDS (6 mL, 6.00 mmol, 24 equiv) dropwise at 0 °C under nitrogen atmosphere. The mixture was stirred for 1 h at 60 °C and then cooled to room temperature. The reaction was quenched with sat. NH₄Cl (aq.) solution (15 mL) and the mixture was extracted with dichloromethane (3×20 mL). The combined organic layers were washed with brine (3×30 mL), dried over anhydrous Na₂SO₄, and concentrated under reduced pressure. The residue was purified by Prep-HPLC with XBridge Shield RP18 OBD column using water containing 0.1% NH₃·H₂O and acetonitrile as mobile phase. Fractions containing the desired compound were evaporated to dryness to give 9.8 mg of 15,21-dimethyl-5-[4-fluoro-3-(N,N-dimethylamino)pyrrolidinyl]-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, isomer 1 (EXAMPLE A84A) as a yellow solid (6%) and 10.0 mg of isomer 2 (EXAMPLE A84B) as a yellow solid (6%).

Isomer 1: LCMS: m/z (ESI), [M+H]⁺=630.40. ¹H NMR (DMSO-d₆, 400 MHz) δ 1.48-1.68 (4H, m), 2.01-2.08 (1H, m), 2.30 (6H, s), 2.41-2.58 (3H, m), 2.55-2.79 (4H, m), 3.04-3.10 (2H, m), 3.22-3.32 (1H, m), 3.54-3.69 (3H, m), 3.62 (3H, s), 3.72 (3H, s), 4.55 (1H, t), 4.62-4.73 (1H, m), 5.25-5.35 (1H, m), 6.50 (1H, dd), 6.76 (1H, s), 7.40 (1H, d), 8.26 (1H, d), 8.42 (1H, s), 8.82 (1H, d), 12.52 (1H, s). SFC-HPLC, Rt=4.025 min.

Isomer 2: LCMS: m/z (ESI), [M+H]⁺=630.40. ¹H NMR (DMSO-d₆, 400 MHz) δ 1.48-1.68 (4H, m), 2.01-2.08 (1H, m), 2.30 (6H, s), 2.41-2.58 (3H, m), 2.55-2.79 (4H, m), 3.04-3.10 (2H, m), 3.22-3.32 (1H, m), 3.54-3.69 (3H, m), 3.62 (3H, s), 3.72 (3H, s), 4.55 (1H, t), 4.62-4.73 (1H, m), 5.25-5.35 (1H, m), 6.59 (1H, dd), 6.86 (1H, s), 7.41 (1H, d), 8.27 (1H, d), 8.42 (1H, s), 8.82 (1H, d), 12.51 (1H, s). SFC-HPLC, Rt=3.757 min.

### Example A84C and Example A84D

### 15,21-Dimethyl-5-[4-fluoro-3-(N,N-dimethylamino)pyrrolidinyl]-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, Isomer 3 (Example A84C) and Isomer 4 (Example A84B)

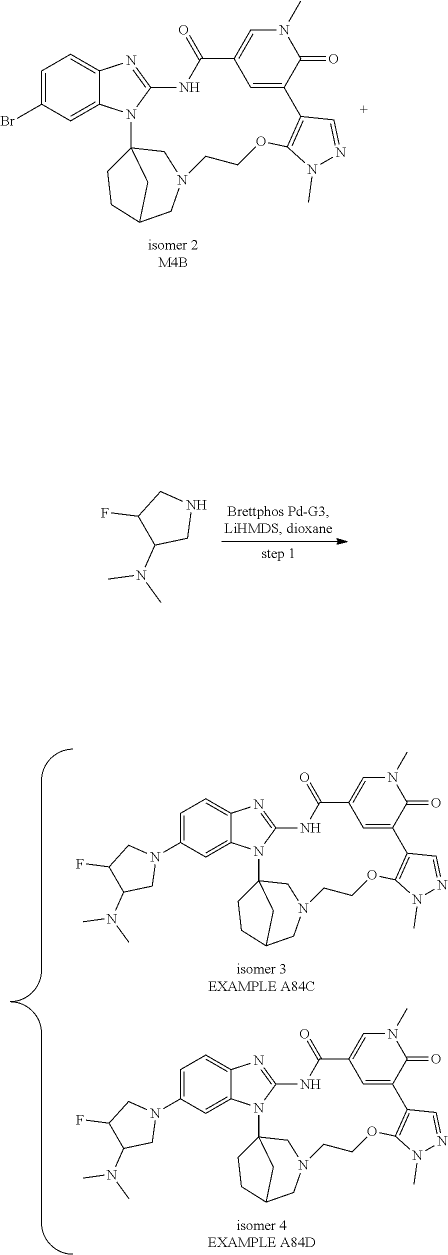

Step 1. Preparation of 15,21-Dimethyl-5-[4-fluoro-3-(N,N-dimethylamino)pyrrolidinyl]-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, isomer 3 (EXAMPLE A84C) and isomer 4 (EXAMPLE A84D). To a stirred mixture of 5-bromo-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3, 8}.0\hat{ }{18,22}]tritriaconta-3,5,7,9,13,17(33),18(22),19-octaene-12,16-dione (M4B, 150 mg, 0.25 mmol, 1 equiv), 4-fluoro-N,N-dimethylpyrrolidin-3-amine (102.83 mg, 0.77 mmol, 3 equiv), and BrettPhos Pd G₃ (70.52 mg, 0.07 mmol, 0.3 equiv) was added LiHMDS (6 mL, 6.00 mmol, 24 equiv) dropwise at 0 °C under nitrogen atmosphere. The mixture was stirred for 1 h at 60 °C and then cooled to room temperature. The reaction was quenched with sat. NH₄Cl (aq.) solution (10 mL) and the mixture was extracted with dichloromethane (3×20 mL). The combined organic layers were washed with brine (3×30 mL), dried over anhydrous Na₂SO₄, and concentrated under reduced pressure. The residue was purified by Prep-HPLC with XBridge Shield RP18 OBD column using water containing 0.10% NH₃·H₂O and acetonitrile as mobile phase. Fractions containing the desired compound were evaporated to dryness to give 11.8 mg of 15,21-dimethyl-5-[4-fluoro-3-(N,N-dimethylamino)pyrrolidinyl]-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, isomer 3 (EXAMPLE A84C) as a yellow solid (7%) and 6.0 mg of isomer 4 (EXAMPLE A84D) as a yellow solid (3%).

Isomer 3: LCMS: m/z (ESI), [M+H]⁺=630.40. ¹H NMR (DMSO-d₆, 400 MHz) δ 1.48-1.68 (4H, m), 2.01-2.08 (1H, m), 2.30 (6H, s), 2.41-2.58 (3H, m), 2.55-2.79 (4H, m), 3.04-3.10 (2H, m), 3.22-3.32 (1H, m), 3.54-3.69 (3H, m), 3.62 (3H, s), 3.72 (3H, s), 4.55 (1H, t), 4.62-4.73 (1H, m), 5.25-5.35 (1H, m), 6.50 (1H, dd), 6.76 (1H, s), 7.40 (1H, d), 8.26 (1H, d), 8.42 (1H, s), 8.82 (1H, d), 12.53 (1H, s). SFC-HPLC, Rt=3.983 min

Isomer 4: LCMS: m/z (ESI), [M+H]⁺=630.40. ¹H NMR (DMSO-d₆, 400 MHz) δ 1.48-1.68 (4H, m), 2.01-2.08 (1H, m), 2.30 (6H, s), 2.41-2.58 (3H, m), 2.55-2.79 (4H, m), 3.04-3.10 (2H, m), 3.22-3.32 (1H, m), 3.54-3.69 (3H, m), 3.62 (3H, s), 3.72 (3H, s), 4.55 (1H, t), 4.62-4.73 (1H, m), 5.25-5.35 (1H, m), 6.59 (1H, dd), 6.86 (1H, s), 7.41 (1H, d), 8.27 (1H, d), 8.42 (1H, s), 8.82 (1H, d), 12.55 (1H, s). SFC-HPLC, Rt=3.724 min.

### Example A94A, Example 94A1, and Example A94A2

### 5-[3-(3-Fluoroazetidin-1-yl)pyrrolidin-1-yl]-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, Isomer A (Example A94A), Isomer A1 (Example A94A1), and Isomer A2 (Example A94A2)

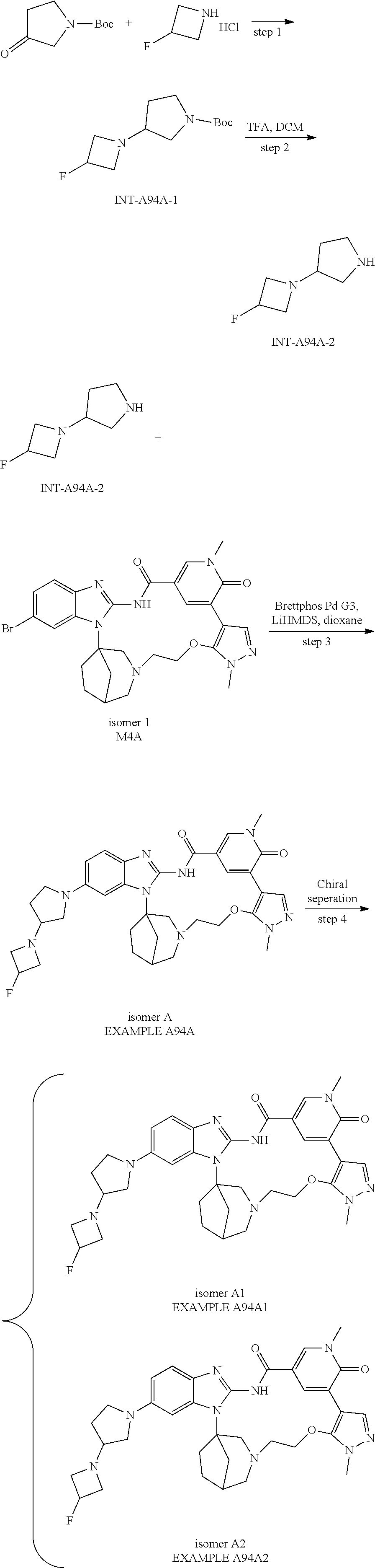

Step 1. Preparation of tert-butyl 3-(3-fluoroazetidin-1-yl)pyrrolidine-1-carboxylate (INT-A94A-1). A mixture of tert-butyl 3-oxopyrrolidine-1-carboxylate (1500 mg, 8.10 mmol, 1 equiv) and 3-fluoroazetidine hydrochloride (903 mg, 8.10 mmol, 1 equiv) in CH₂Cl₂ (50 mL) was stirred for 1 h at room temperature under air atmosphere. To the above mixture was added NaBH(OAc)₃ (8582 mg, 40.49 mmol, 5 equiv) in portions over 2 min at 0 °C. The resulting mixture was stirred for additional 2 h. The reaction was quenched with sat. NH₄Cl (aq.) (50 mL) and the mixture was extracted with CH₂Cl₂ (3×100 mL). The combined organic layers were washed with brine (3×100 mL), dried over anhydrous Na₂SO₄, and concentrated under reduced pressure. The residue was purified on a silica gel column eluted with dichloromethane/methanol (12/1) to give 1.5 g of tert-butyl 3-(3-fluoroazetidin-1-yl)pyrrolidine-1-carboxylate (INT-A94A-1) as a yellow oil (75%). ¹H NMR (CDCl₃, 400 MHz) δ 1.46 (9H, d), 1.68-1.83 (1H, m), 1.85-1.95 (1H, m), 3.05-3.15 (1H, m), 3.18-3.48 (6H, m), 3.72-3.87 (2H, m)

Step 2. Preparation of 3-(3-fluoroazetidin-1-yl)pyrrolidine (INT-A94A-2). A solution of tert-butyl 3-(3-fluoroazetidin-1-yl)pyrrolidine-1-carboxylate (500 mg, 2.05 mmol, 1 equiv) and trifluoroacetic acid (3 mL) in CH₂Cl₂ (9 mL) was stirred for 2 h at room temperature. The mixture was concentrated under reduced pressure, treated with dichloromethane/methanol (10/1) (30 mL), and adjusted to pH 7 with K₂CO₃. The mixture was filtered and the filter cake was washed with methanol (2×10 mL). The resulting solution was concentrated under reduced pressure to give 150 mg of 3-(3-fluoroazetidin-1-yl)pyrrolidine (INT-A94A-2) as a yellow oil. This material was used for the next reaction without further purification. ¹H NMR (DMSO-dd, 400 MHz) δ 1.75-1.84 (2H, m), 2.70-2.81 (2H, m), 2.82-2.95 (2H, m), 3.05-3.21 (3H, m), 3.59-3.62 (2H, m), 4.98-5.18 (2H, m)

Step 3. Preparation of 5-[3-(3-fluoroazetidin-1-yl)pyrrolidin-1-yl]-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (isomer A, EXAMPLE A94A). To a stirred mixture of 3-(3-fluoroazetidin-1-yl)pyrrolidine (125 mg, 0.86 mmol, 5 equiv), BrettPhos Pd G₃ (47 mg, 0.05 mmol, 0.3 equiv), and 5-bromo-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (M4A, 100 mg, 0.173 mmol, 1 equiv) in 1,4-dioxane (10 mL) was added LiHMDS (2.1 mL, 2.07 mmol, 12 equiv). The resulting mixture was stirred for 2 h at 60 °C under nitrogen atmosphere. The mixture was cooled to room temperature. The reaction was quenched with sat. NH₄Cl (aq.) (15 mL) and the mixture was extracted with CH₂Cl₂ (3×50 mL). The combined organic layers were washed with brine (3×30 mL), dried over anhydrous Na₂SO₄, and concentrated under reduced pressure. The residue was purified by Prep-TLC eluted with dichloromethane/methanol (10:1). The product was further purified by Prep-HPLC with XBridge Prep Phenyl OBD column using water containing 0.10% NH₄HCO₃ and acetonitrile as mobile phase. Fractions containing the desired compound were evaporated to dryness to give 13 mg of 5-[3-(3-fluoroazetidin-1-yl)pyrrolidin-1-yl]-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, isomer A (EXAMPLE A94A) as an off-white solid (12%). LCMS: m/z (ESI), [M+H]⁺=642.40. ¹H NMR (DMSO-d₆, 400 MHz) δ 1.48-1.85 (4H, m), 1.89-2.05 (2H, m), 2.35-2.56 (4H, m), 2.68-2.76 (2H, m), 2.96 (1H, t), 3.07-3.20 (4H, m), 3.20-3.43 (4H, m), 3.45-3.70 (3H, m), 3.62 (3H, s), 3.72 (3H, s), 4.55 (1H, t), 4.63-4.73 (1H, m), 4.95-5.31 (1H, m), 6.44 (1H, d), 6.73 (1H, s), 7.37 (1H, d), 8.25 (1H, d), 8.42 (1H, s), 8.82 (1H, d), 12.50 (1H, s).

Step 4. Preparation of 5-[3-(3-fluoroazetidin-1-yl)pyrrolidin-1-yl]-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, isomer A1 (EXAMPLE A94A1) and isomer A2 (EXAMPLE A94A2). The mixture of 5-[3-(3-fluoroazetidin-1-yl)pyrrolidin-1-yl]-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (EXAMPLE A94A, 10.5 mg) was separated by Prep-Chiral-HPLC with CHIRALPAK IG column (2×25 cm, 5 μm) using hexane containing 0.2% diethylamine and ethanol/CH₂Cl₂ (1/1) as mobile phase to give 2.3 mg of 5-[3-(3-fluoroazetidin-1-yl)pyrrolidin-1-yl]-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, isomer A1 (EXAMPLE A94A1) as a yellow solid (21%) and 3.4 mg of isomer A2 (EXAMPLE A94A2) as a yellow solid (32%).

Isomer A1: LCMS: m/z (ESI), [M+H]⁺=642.35 ¹H NMR (DMSO-d₆, 400 MHz) δ 1.48-1.85 (4H, m), 1.89-2.05 (2H, m), 2.35-2.56 (4H, m), 2.68-2.76 (2H, m), 2.96 (1H, t), 3.07-3.20 (4H, m), 3.20-3.43 (4H, m), 3.45-3.70 (3H, m), 3.62 (3H, s), 3.72 (3H, s), 4.55 (1H, t), 4.63-4.73 (1H, m), 4.95-5.31 (11H, m), 6.44 (1H, d), 6.73 (1H, s), 7.37 (11H, d), 8.25 (1H, d), 8.42 (1H, s), 8.82 (1H, d), 12.50 (1H, s). Chiral-HPLC, Rt=2.381 min.

Isomer A2: LCMS: m/z (ESI), [M+H]⁺=642.55. ¹H NMR (DMSO-d₆, 400 MHz) δ 1.48-1.85 (4H, m), 1.89-2.05 (2H, m), 2.35-2.56 (4H, m), 2.68-2.76 (2H, m), 2.96 (1H, t), 3.07-3.20 (4H, m), 3.20-3.43 (4H, m), 3.45-3.70 (3H, m), 3.62 (3H, s), 3.72 (3H, s), 4.55 (1H, t), 4.63-4.73 (1H, m), 4.95-5.31 (11H, m), 6.44 (1H, d), 6.73 (1H, s), 7.37 (11H, d), 8.25 (1H, d), 8.42 (1H, s), 8.82 (1H, d), 12.50 (1H, s). Chiral-HPLC, Rt=2.959 min.

### Example A94B, Example 94B1, and Example A94B2

### 5-[3-(3-Fluoroazetidin-1-yl)pyrrolidin-1-yl]-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, Isomer B (Example A94B), Isomer B1 (Example A94B1), and Isomer B2 (Example A94B2)

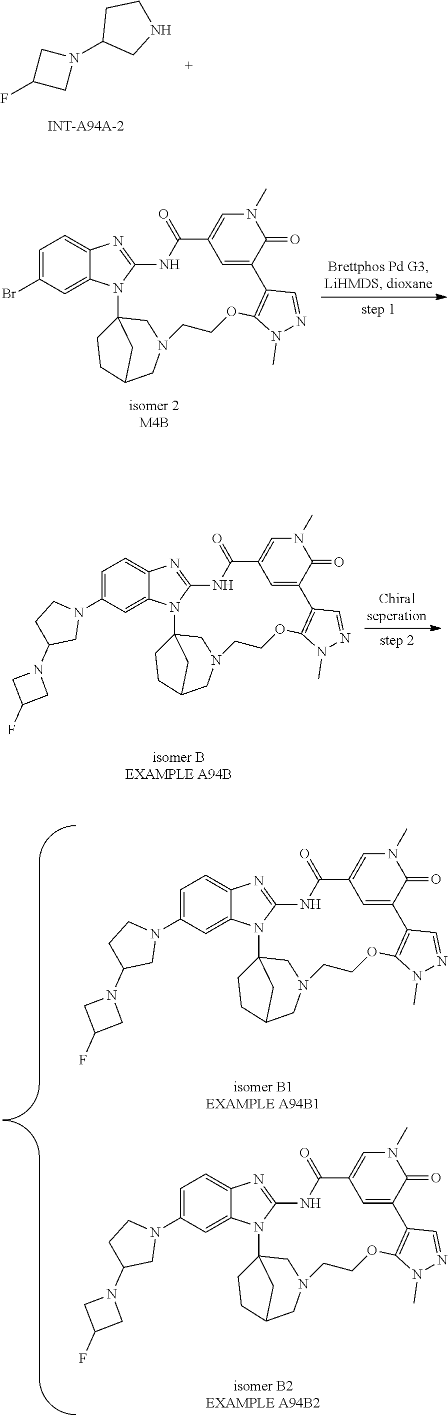 [SMILES: FC1CN(C2CCNC2)C1 ; Cn1ncc2c1OCCN1CC3CCC(C3)(C1)n1c(nc3ccc(Br)cc31)NC(=O)c1cc-2c(=O)n(C)c1 ; Cn1ncc2c1OCCN1CC3CCC(C3)(C1)n1c(nc3ccc(N4CCC(N5CC(F)C5)C4)cc31)NC(=O)c1cc-2c(=O)n(C)c1 ; Cn1ncc2c1OCCN1CC3CCC(C3)(C1)n1c(nc3ccc(N4CCC(N5CC(F)C5)C4)cc31)NC(=O)c1cc-2c(=O)n(C)c1 ; Cn1ncc2c1OCCN1CC3CCC(C3)(C1)n1c(nc3ccc(N4CCC(N5CC(F)C5)C4)cc31)NC(=O)c1cc-2c(=O)n(C)c1]

Step 1. Preparation of 5-[3-(3-fluoroazetidin-1-yl)pyrrolidin-1-yl]-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (isomer B, EXAMPLE A94B). To a stirred mixture of 3-(3-fluoroazetidin-1-yl)pyrrolidine (INT-A94A-2, 125 mg, 0.86 mmol, 5 equiv), BrettPhos Pd G₃ (47 mg, 0.05 mmol, 0.3 equiv) and 5-bromo-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (M4B, 100 mg, 0.173 mmol, 1 equiv) in 1,4-dioxane (10 mL) was added LiHMDS (2.1 mL, 2.07 mmol, 12 equiv). The resulting mixture was stirred for 2 h at 60 °C under nitrogen atmosphere. The mixture was cooled to room temperature. The reaction was quenched with sat.NH₄Cl (aq.) (15 mL) and the mixture was extracted with CH₂Cl₂ (3×50 mL). The combined organic layers were washed with brine (3×30 mL), dried over anhydrous Na₂SO₄, and concentrated under reduced pressure. The residue was purified by Prep-TLC eluted with dichloromethane/methanol (10/1). The product was further purified by Prep-HPLC with XBridge Prep OBD C18 column using water containing 0.10% NH₄HCO₃ and acetonitrile as mobile phase. Fractions containing the desired compound were evaporated to dryness to give 15.3 mg of 5-[3-(3-fluoroazetidin-1-yl)pyrrolidin-1-yl]-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, isomer B (EXAMPLE A94B) as an off-white solid (13%). LCMS: m/z (ESI), [M+H]⁺=642.40. ¹H NMR (DMSO-d₆, 400 MHz) δ 1.48-1.85 (4H, m), 1.89-2.05 (2H, m), 2.35-2.56 (4H, m), 2.68-2.76 (2H, m), 2.96 (1H, t), 3.07-3.20 (4H, m), 3.20-3.43 (4H, m), 3.45-3.70 (3H, m), 3.62 (3H, s), 3.72 (3H, S), 4.55 (1H, t), 4.63-4.73 (1H, m), 4.95-5.31 (1H, m), 6.44 (1H, d), 6.73 (1H, s), 7.37 (1H, d), 8.25 (1H, d), 8.42 (1H, s), 8.82 (1H, d), 12.50 (1H, s).

Step 2. Preparation of 5-[3-(3-fluoroazetidin-1-yl)pyrrolidin-1-yl]-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, isomer B1 (EXAMPLE A94B1) and isomer B2 (EXAMPLE A94B2). The mixture of 5-[3-(3-fluoroazetidin-1-yl)pyrrolidin-1-yl]-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (EXAMPLE A94B, 11 mg) was separated by Prep-Chiral-HPLC with CHIRALPAK IG column (2×25 cm, 5 μm) using hexane containing 0.2% diethylamine as mobile phase A and ethanol/CH₂Cl₂ (1/1) as mobile phase B give 3.7 mg of 5-[3-(3-fluoroazetidin-1-yl)pyrrolidin-1-yl]-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, isomer B1 (EXAMPLE A94B1) as a yellow solid (28%) and 4.5 mg of isomer B2 (EXAMPLE A94B2) as a yellow solid (34%).

Isomer B1: LCMS: m/z (ESI), [M+H]⁺=642.30. ¹H NMR (DMSO-d₆, 400 MHz) δ 1.48-1.85 (4H, m), 1.89-2.05 (2H, m), 2.35-2.56 (4H, m), 2.68-2.76 (2H, m), 2.96 (1H, t), 3.07-3.20 (4H, m), 3.20-3.43 (4H, m), 3.45-3.70 (3H, m), 3.62 (3H, s), 3.72 (3H, S), 4.55 (1H, t), 4.63-4.73 (1H, m), 4.95-5.31 (1H, m), 6.44 (1H, d), 6.73 (1H, s), 7.37 (1H, d), 8.25 (1H, d), 8.42 (1H, s), 8.82 (1H, d), 12.50 (1H, s). Chiral-HPLC, Rt=1.608 min.

Isomer B2: LCMS: m/z (ESI), [M+H]⁺=642.55. ¹H NMR (DMSO-d₆, 400 MHz) δ 1.48-1.85 (4H, m), 1.89-2.05 (2H, m), 2.35-2.56 (4H, m), 2.68-2.76 (2H, m), 2.96 (1H, t), 3.07-3.20 (4H, m), 3.20-3.43 (4H, m), 3.45-3.70 (3H, m), 3.62 (3H, s), 3.72 (3H, S), 4.55 (1H, t), 4.63-4.73 (1H, m), 4.95-5.31 (1H, m), 6.48 (1H, d), 6.76 (1H, s), 7.38 (1H, d), 8.26 (1H, d), 8.42 (1H, s), 8.82 (1H, d), 12.52 (1H, s). Chiral-HPLC, Rt=2.068 min.

### Example A100

### 15,21-Dimethyl-23-oxa-2,9,11,15,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,17(34),18(22),19-octaene-12,16-dione

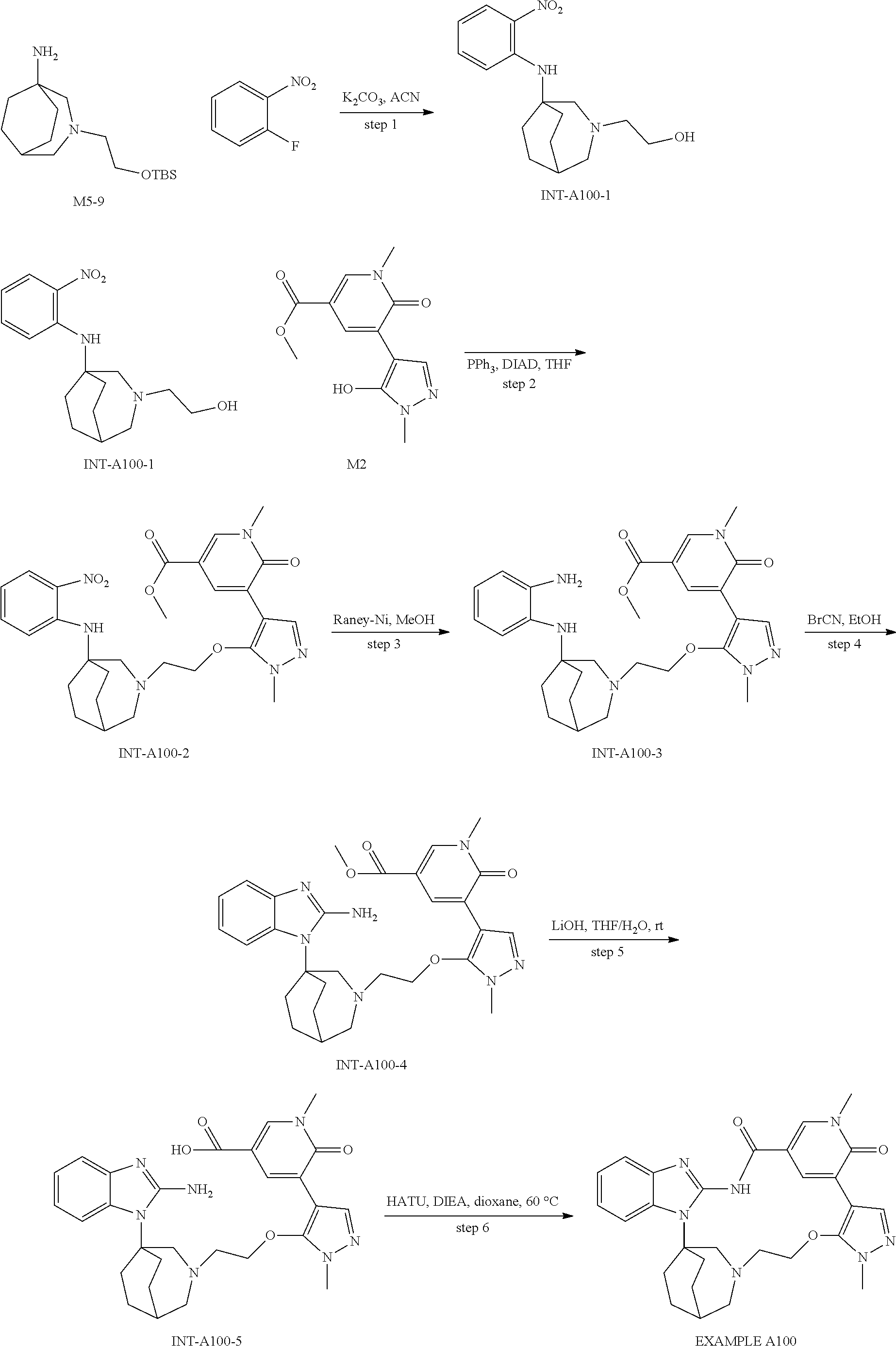

Step 1. Preparation of 2-{1-[(2-nitrophenyl)amino]-3-azabicyclo[3.2.2]nonan-3-yl}ethanol (INT-A100-1). A mixture of 3-{2-[(tert-butyldimethylsilyl)oxy]ethyl}-3-azabicyclo[3.2.2]nonan-1-amine (250 mg, 0.83 mmol, 1 equiv), K₂CO₃ (347 mg, 2.51 mmol, 3 equiv) and O-fluoronitrobenzene (295 mg, 2.09 mmol, 2.5 equiv) in acetonitrile (8 mL) was stirred for 8 h at 100 °C under nitrogen atmosphere. The mixture was cooled to room temperature. The reaction was quenched with water (20 mL) and the mixture was extracted with ethyl acetate (3×30 mL). The combined organic layers were washed with brine (3×50 mL), dried over anhydrous Na₂SO₄, and concentrated under reduced pressure. The residue was purified on a silica gel column eluted with petroleum ether/ethyl acetate (5/1) to give 110 mg of 2-{1-[(2-nitrophenyl)amino]-3-azabicyclo[3.2.2]nonan-3-yl}ethanol (INT-A100-1) as a yellow oil (38%). LCMS: m/z (ESI), [M+H]⁺=306.15.

Step 2. Preparation of methyl 1-methyl-5-[1-methyl-5-(2-{1-[(2-nitrophenyl)amino]-3-azabicyclo[3.2.2]nonan-3-yl}ethoxy)pyrazol-4-yl]-6-oxopyridine-3-carboxylate (INT-A100-2). To a stirred mixture of 2-{l-[(2-nitrophenyl)amino]-3-azabicyclo[3.2.2]nonan-3-yl}ethanol (100 mg, 0.32 mmol, 1 equiv) and triphenylphosphine (257 mg, 0.98 mmol, 3 equiv) in tetrahydrofuran (20 mL) were added diisopropyl azodicarboxylate (198 mg, 0.98 mmol, 3 equiv), and methyl 5-(5-hydroxy-1-methylpyrazol-4-yl)-1-methyl-6-oxopyridine-3-carboxylate (M2, 68 mg, 0.26 mmol, 0.8 equiv) in tetrahydrofuran (5 mL) dropwise at 0 °C under nitrogen atmosphere. The mixture was stirred at 0 °C for 2 h. The reaction was quenched with water (20 mL) and the mixture was extracted with ethyl acetate (3×30 mL). The combined organic layers were washed with brine (3×50 mL), dried over anhydrous Na₂SO₄, and concentrated under reduced pressure. The residue was purified on a silica gel column eluted with petroleum ether/ethyl acetate (1/1) to give 150 mg of methyl 1-methyl-5-[1-methyl-5-(2-{1-[(2-nitrophenyl)amino]-3-azabicyclo[3.2.2]nonan-3-yl}ethoxy)pyrazol-4-yl]-6-oxopyridine-3-carboxylate (INT-A100-2) as a yellow oil (74%). LCMS: m/z (ESI), [M+H]⁺=551.20.

Step 3. Preparation of methyl 5-[5-(2-{1-[(2-aminophenyl)amino]-3-azabicyclo[3.2.2]nonan-3-yl}ethoxy)-1-methylpyrazol-4-yl]-1-methyl-6-oxopyridine-3-carboxylate (INT-A100-3). To a stirred mixture of methyl 1-methyl-5-[1-methyl-5-(2-{1-[(2-nitrophenyl)amino]-3-azabicyclo[3.2.2]nonan-3-yl}ethoxy)pyrazol-4-yl]-6-oxopyridine-3-carboxylate (140 mg, 0.25 mmol, 1 equiv) and Raney Nickel (69 mg) in methanol (5 mL) was added hydrazine hydrate (51 mg, 1.01 mmol, 4.00 equiv) at 0 °C. The mixture was stirred for 2 h at 0 °C under nitrogen atmosphere. The resulting mixture was filtered and the filter cake was washed with methanol (3×10 mL). The resulting solution was concentrated under reduced pressure to give 90 mg of methyl 5-[5-(2-{1-[(2-aminophenyl)amino]-3-azabicyclo[3.2.2]nonan-3-yl}ethoxy)-1-methylpyrazol-4-yl]-1-methyl-6-oxopyridine-3-carboxylate (INT-A100-3) as a light yellow solid (58%). LCMS: m/z (ESI), [M+H]⁺=521.30.

Step 4. Preparation of methyl 5-(5-{2-[1-(2-amino-1,3-benzodiazol-1-yl)-3-azabicyclo[3.2.2]nonan-3-yl]ethoxy}-1-methylpyrazol-4-yl)-1-methyl-6-oxopyridine-3-carboxylate (INT-A100-4). A mixture of methyl 5-[5-(2-{1-[(2-aminophenyl)amino]-3-azabicyclo[3.2.2]nonan-3-yl}ethoxy)-1-methylpyrazol-4-yl]-1-methyl-6-oxopyridine-3-carboxylate (90 mg, 0.17 mmol, 1 equiv) and BrCN (21 mg, 0.20 mmol, 1.20 equiv) in ethanol (5 mL) was stirred overnight at room temperature under nitrogen atmosphere. The reaction was quenched with water (20 mL) and the mixture was extracted with ethyl acetate (3×30 mL). The combined organic layers were washed with brine (3×50 mL), dried over anhydrous Na₂SO₄, and concentrated under reduced pressure. The residue was purified by Prep-TLC eluted with petroleum ether/ethyl acetate (1/1) to give 60 mg of methyl 5-(5-{2-[1-(2-amino-1,3-benzodiazol-1-yl)-3-azabicyclo[3.2.2]nonan-3-yl]ethoxy}-1-methylpyrazol-4-yl)-1-methyl-6-oxopyridine-3-carboxylate (INT-A100-4) as a yellow solid (57%). LCMS: m/z (ESI), [M+H]⁺=546.35.

Step 5. Preparation of 5-(5-{2-[1-(2-amino-1,3-benzodiazol-1-yl)-3-azabicyclo[3.2.2]nonan-3-yl]ethoxy}-1-methylpyrazol-4-yl)-1-methyl-6-oxopyridine-3-carboxylic acid (INT-A100-5). A mixture of methyl 5-(5-{2-[1-(2-amino-1,3-benzodiazol-1-yl)-3-azabicyclo[3.2.2]nonan-3-yl]ethoxy}-1-methylpyrazol-4-yl)-1-methyl-6-oxopyridine-3-carboxylate (50 mg, 0.09 mmol, 1 equiv) and LiOH·H₂O (7 mg, 0.27 mmol, 3 equiv) in tetrahydrofuran (4 mL) and water (1 mL) was stirred for 2 h at room temperature under nitrogen atmosphere. The mixture was concentrated under reduced pressure. The residue was purified by C18 Flash using water containing 10 mmol/L NH₄HCO₃ and 0.10% NH₃·H₂O and acetonitrile as mobile phase. Fractions containing the desired compound were evaporated to dryness to give 25 mg of 5-(5-{2-[1-(2-amino-1,3-benzodiazol-1-yl)-3-azabicyclo[3.2.2]nonan-3-yl]ethoxy}-1-methylpyrazol-4-yl)-1-methyl-6-oxopyridine-3-carboxylic acid (INT-A100-5) as a yellow solid (50%). LCMS: m/z (EST), [M+H]⁺=532.25.

Step 6. Preparation of 15,21-dimethyl-23-oxa-2,9,11,15,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,17(34),18(22),19-octaene-12,16-dione (EXAMPLE A100). A mixture of 5-(5-{2-[1-(2-amino-1,3-benzodiazol-1-yl)-3-azabicyclo[3.2.2]nonan-3-yl]ethoxy}-1-methylpyrazol-4-yl)-1-methyl-6-oxopyridine-3-carboxylic acid (30 mg, 0.05 mmol, 1 equiv), N,N,N,N-tetramethyl-O-(7-azabenzotriazol-1-yl)uronium hexafluorophospate (32 mg, 0.08 mmol, 1.5 equiv), and N,N-diisopropylethylamine (21 mg, 0.16 mmol, 3 equiv) in 1,4-dioxane (5 mL) was stirred for 2 h at 60 °C under nitrogen atmosphere. The mixture was cooled to room temperature. The reaction was quenched with water (10 mL) and the mixture was extracted with ethyl acetate (3×20 mL). The combined organic layers were washed with brine (3×30 mL), dried over anhydrous Na₂SO₄, and concentrated under reduced pressure. The residue was purified by Prep-HPLC with XBridge Shield RP18 OBD column using water containing 10 mmol/L NH₄HCO₃ and 0.1% NH₃—H₂O and acetonitrile as mobile phase. Fractions containing the desired compound were evaporated to dryness to give 26.8 mg of 15,21-dimethyl-23-oxa-2,9,11,15,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,17(34),18(22),19-octaene-12,16-dione (EXAMPLE A100) as a white solid (92%). LCMS: m/z (EST), [M+H]⁺=514.20. ¹H NMR (DMSO-d₆, 400 MHz) δ 1.85-1.98 (4H, m), 2.02-2.18 (3H, m), 2.78-2.88 (2H, m), 2.88-2.95 (2H, m), 3.40-3.52 (4H, m), 3.63 (3H, s), 3.75 (3H, s), 4.29 (2H, s), 7.12-7.21 (2H, m), 7.63 (1H, d), 7.89 (1H, d), 8.38-8.24 (2H, m), 9.12 (1H, d), 13.30 (1H, s).

### Example A101

### (11R)-15-Chloro-5,11,26-trimethyl-16-(4-methylpiperazin-1-yl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione

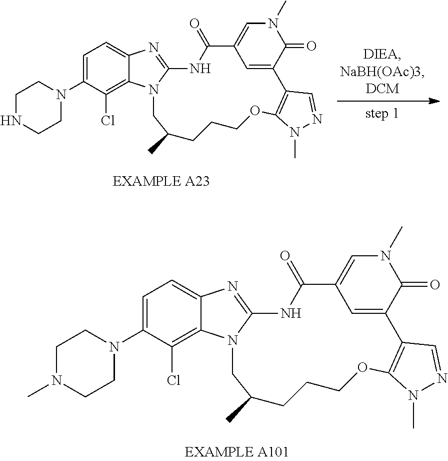 [SMILES: C[C@@H]1CCCOc2c(cnn2C)-c2cc(cn(C)c2=O)C(=O)Nc2nc3ccc(N4CCNCC4)c(Cl)c3n2C1 ; C[C@@H]1CCCOc2c(cnn2C)-c2cc(cn(C)c2=O)C(=O)Nc2nc3ccc(N4CCN(C)CC4)c(Cl)c3n2C1]

Step 1. Preparation of (11R)-15-chloro-5,11,26-trimethyl-16-(4-methylpiperazin-1-yl)-7-oxa-4,5,13,20,22,26-hexaazapentacyclo[22.3.1.0\hat{ }{2,6}.0\hat{ }{13,21}.0′{14,19}]octacosa-1(28),2(6),3,14,16,18,20,24-octaene-23,27-dione (EXAMPLE A101). To a stirred mixture of (11R)-15-chloro-5,11,26-trimethyl-16-(piperazin-1-yl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (EXAMPLE A23, 80 mg, 0.14 mmol, 1 equiv), paraformaldehyde (38 mg, 0.42 mmol, 3 equiv) and NaBH(OAc)₃ (60 mg, 0.28 mmol, 2 equiv) in CH₂Cl₂ (20 mL) was added N,N-diisopropylethylamine (36 mg, 0.28 mmol, 2 equiv) dropwise at 0 °C under nitrogen atmosphere. The mixture was stirred for 2 h at 0 °C. The reaction was quenched with water (10 mL) and the mixture was extracted with CH₂Cl₂ (3×10 mL). The combined organic layers were washed with brine (3×10 mL), dried over anhydrous Na₂SO₄, and concentrated under reduced pressure. The residue was purified by Prep-HPLC with XBridge Shield RP18 OBD column using water containing 10 mmol/L NH₄HCO₃ and 0.10% NH₃·H₂O and acetonitrile as mobile phase. Fractions containing the desired compound were evaporated to dryness to give 32.4 mg of (11R)-15-chloro-5,11,26-trimethyl-16-(4-methylpiperazin-1-yl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (EXAMPLE A101) as a white solid (39%). LCMS: m/z (ESI), [M+H]⁺=579.20. ¹H NMR (DMSO-d₆, 400 MHz) δ 0.86 (3H, d), 1.51-1.59 (1H, m), 1.86-2.08 (2H, m), 2.21-2.29 (5H, m), 2.51-2.64 (4H, m), 2.80-2.94 (4H, m), 3.62 (3H, s), 3.71 (3H, s), 3.91-4.03 (1H, m), 4.18 (1H, dd), 4.32-4.49 (1H, m), 4.75-4.97 (1H, m), 7.15 (1H, d), 7.58 (1H, d), 8.31-8.47 (2H, m), 8.85 (1H, d), 12.73 (1H, s).

### Example A102 and Example A103

### (11R)-5,11,30-Trimethyl-7-oxa-4,5,13,18,24,26,30-heptaazahexacyclo[26.3.1.0\hat{ }{2,6}.0\hat{ }{13,25}.0\hat{ }{14,23}.0\hat{ }{15,20}]dotriaconta-1(32),2(6),3,14,20,22,24,28-octaene-27,31-dione (Example A102) and (11R)-5,11,18,30-tetramethyl-7-oxa-4,5,13,18,24,26,30-heptaazahexacyclo[26.3.1.0\hat{ }{2,6}.0\hat{ }{13,25}.0\hat{ }{14,23}.0\hat{ }{15,20}]dotriaconta-1(32),2(6),3,14,20,22,24,28-octaene-27,31-dione (Example A103)

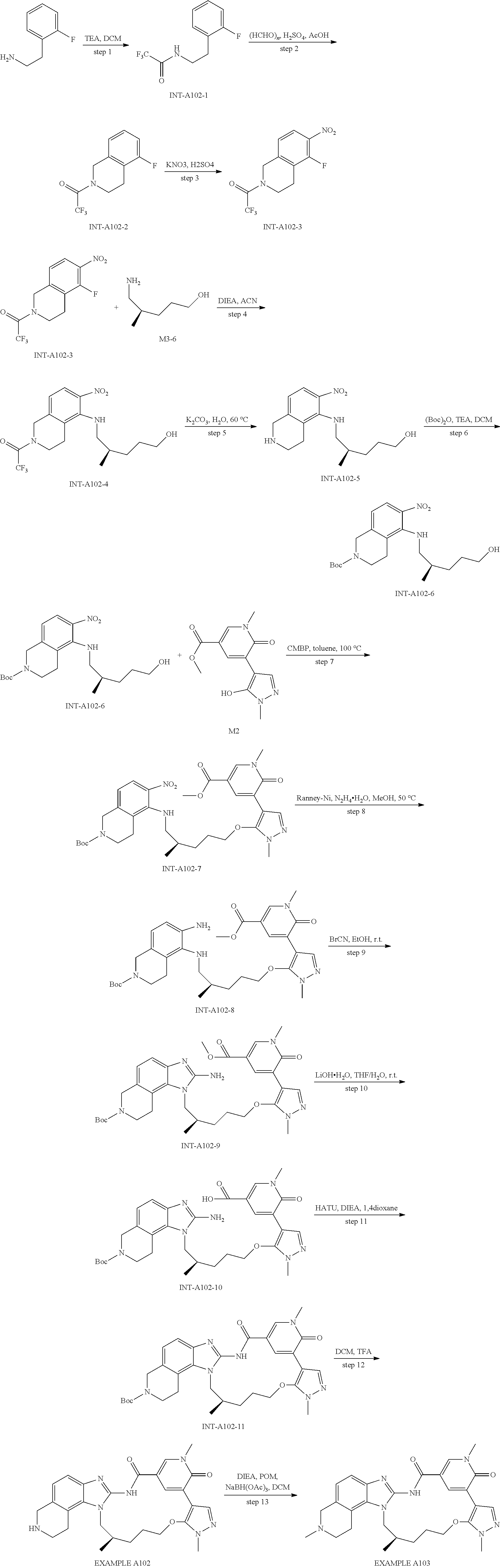

Step 1. Preparation of 2,2,2-trifluoro-N-(2-fluorophenethyl)acetamide (INT-A102-1). A mixture of 2-(2-fluorophenyl)ethanamine (50 g, 359 mmol, 1 equiv), trimethylamine (73 g, 359 mmol, 2 equiv) and trifluoroacetic anhydride (75 g, 1.1 equiv) in dichloromethane (800 mL) was stirred for 3 h at 0 °C under nitrogen atmosphere. The reaction was quenched with water (200 mL) and the mixture was extracted with dichloromethane (3×200 mL). The combined organic layers were washed with brine (3×200 mL), dried over anhydrous Na₂SO₄, and concentrated under reduced pressure. The residue was purified by silica gel chromatography eluted with petroleum ether/ethyl acetate (5/1) to give 76 g of 2,2,2-trifluoro-N-[2-(2-fluorophenyl)ethyl]acetamide (INT-A102-1) as a yellow oil (90%). LCMS: m/z (EST), [M+H]⁺=236.05.

Step 2. Preparation of 2,2,2-trifluoro-1-(5-fluoro-3,4-dihydroisoquinolin-2(1H)-yl)ethan-1-one (INT-A102-2). A mixture of 2,2,2-trifluoro-N-[2-(2-fluorophenyl)ethyl]acetamide (76 g, 323.15 mmol, 1 equiv), (HCHO)ₙ (43 g, 969.5 mmol, 3 equiv), and H₂SO₄ (224 mL, 4201 mmol, 13 equiv) and AcOH (296 mL) was stirred for 3 h at 0 °C under nitrogen atmosphere. The reaction was quenched with water (800 mL) and neutralized to pH 7 with NaOH. The mixture was extracted with dichloromethane (3×500 mL). The combined organic layers were washed with brine (3×500 mL), dried over anhydrous Na₂SO₄ and concentrated under reduced pressure. The residue was purified by silica gel chromatography eluted with petroleum ether/ethyl acetate (1/1) to give 10 g of 2,2,2-trifluoro-1-(5-fluoro-3,4-dihydro-1H-isoquinolin-2-yl)ethanone (INT-A102-2) as a brown yellow oil (13%). LCMS: m/z (ESI), [M+H]⁺=248.10.

Step 3. Preparation of 2,2,2-trifluoro-1-(5-fluoro-6-nitro-3,4-dihydroisoquinolin-2(1H)-yl)ethan-1-one (INT-A102-3). A mixture of 2,2,2-trifluoro-1-(5-fluoro-3,4-dihydro-1H-isoquinolin-2-yl)ethanone (10 g, 40.45 mmol, 1 equiv) and KNO₃ (4 g, 40.45 mmol, 1 equiv) in H₂SO₄ (100 mL) was stirred overnight at 0 °C. The reaction was quenched with water (500 mL) and adjusted to pH 9 with NaOH. The mixture was extracted with dichloromethane (3×200 mL). The combined organic layers were washed with brine (3×200 mL), dried over anhydrous Na₂SO₄, and concentrated under reduced pressure. The residue was purified by silica gel chromatography eluted with petroleum ether/ethyl acetate (3/1) to give 1.4 g of 2,2,2-trifluoro-1-(5-fluoro-6-nitro-3,4-dihydro-1H-isoquinolin-2-yl)ethanone (INT-A102-3) as a yellow oil (12%). ¹H NMR (DMSO, 400 MHz) δ 2.91-2.99 (2H, m), 3.85-3.92 (2H, m), 4.91 (2H, d), 7.45 (1H, d), 8.12 (1H, d).

Step 4. Preparation of 2,2,2-trifluoro-1-(5-{[(2R)-5-hydroxy-2-methylpentyl]amino}-6-nitro-3,4-dihydro-1H-isoquinolin-2-yl)ethanone (INT-A102-4). A mixture of 2,2,2-trifluoro-1-(5-fluoro-6-nitro-3,4-dihydro-1H-isoquinolin-2-yl)ethanone (INT-A102-3, 15 g, 51.34 mmol, 1 equiv), N,N-diisopropylethylamine (19.9 g, 154.01 mmol, 3 equiv), and (4R)-5-amino-4-methylpentan-1-ol (M3-6, 6016 mg, 51.34 mmol, 1 equiv) in acetonitrile (150 mL) was stirred for 2 h at 80 °C. The mixture was cooled to room temperature. The reaction was quenched with water (500 mL) and the mixture was extracted with dichloromethane (3×500 mL). The combined organic layers were washed with brine (3×100 mL), dried over anhydrous Na₂SO₄, and concentrated under reduced pressure. The residue was purified by silica gel column eluted with petroleum ether/ethyl acetate (1/1) to give 10 g of 2,2,2-trifluoro-1-(5-{[(2R)-5-hydroxy-2-methylpentyl]amino}-6-nitro-3,4-dihydro-1H-isoquinolin-2-yl)ethanone (INT-A102-3) as a yellow solid (50%). LCMS: m/z (ESI), [M+H]⁺=390.10.

Step 5. Preparation of (4R)-4-methyl-5-[(6-nitro-1,2,3,4-tetrahydroisoquinolin-5-yl)amino]pentan-1-ol (INT-A102-5). A mixture of 2,2,2-trifluoro-1-(5-{[(2R)-5-hydroxy-2-methylpentyl]amino}-6-nitro-3,4-dihydro-1H-isoquinolin-2-yl)ethanone (10 g, 25.68 mmol, 1 equiv) and K₂CO₃ (10.6 g, 77.05 mmol, 3 equiv) in methanol (80 mL) and H₂O (40 mL) was stirred for 5 h at room temperature. The reaction was treated with water (500 mL) and the mixture was extracted with dichloromethane (3×500 mL). The combined organic layers were washed with brine (3×100 mL), dried over anhydrous Na₂SO₄, and concentrated under reduced pressure. The residue was purified by silica gel column, eluted with petroleum ether/ethyl acetate (1/1) to give 7 g of (4R)-4-methyl-5-[(6-nitro-1,2,3,4-tetrahydroisoquinolin-5-yl)amino]pentan-1-ol (INT-A102-5) as a yellow solid (93%). LCMS: m/z (EST), [M+H]⁺=294.15. ¹H NMR (DMSO-d₆,400 MHz) δ 0.83-0.88 (3H, m), 1.07-1.11 (1H, m), 1.22-1.51 (3H, m), 1.57-1.64 (1H, m), 2.59 (2H, t), 2.88-2.92 (3H, m), 3.10-3.20 (1H, m), 3.30-3.33 (2H, m), 3.87 (2H, s), 4.37 (1H, s), 6.56 (1H, d), 6.69 (1H, t), 7.26-7.37 (1H, m), 7.70 (1H, d).

Step 6. Preparation of tert-butyl 5-{[(2R)-5-hydroxy-2-methylpentyl]amino}-6-nitro-3,4-dihydro-1H-isoquinoline-2-carboxylate (INT-A102-6). A mixture of (4R)-4-methyl-5-[(6-nitro-1,2,3,4-tetrahydroisoquinolin-5-yl)amino]pentan-1-ol (8 g, 27.27 mmol, 1 equiv), trimethylamine (5.5 g, 54.54 mmol, 2 equiv) and di-tert-butyl dicarbonate (7.1 g, 32.72 mmol, 1.2 equiv) in dichloromethane (100 mL) was stirred for 2 h at room temperature. The reaction was quenched with water (300 mL) and the mixture was extracted with dichloromethane (3×300 mL). The combined organic layers were washed with brine (3×100 mL), dried over anhydrous Na₂SO₄, and concentrated under reduced pressure. The residue was purified by silica gel column eluted with petroleum ether/ethyl acetate (1/1) to give 6.5 g of tert-butyl 5-{[(2R)-5-hydroxy-2-methylpentyl]amino}-6-nitro-3,4-dihydro-1H-isoquinoline-2-carboxylate (INT-A102-6) as a yellow oil (60%). LCMS: m/z (ESI), [M+H]⁺=394.20. ¹H NMR (DMSO-d₆ 400 MHz) δ 0.84 (3H, d), 1.03-1.17 (1H, m), 1.22-1.45 (2H, m), 1.45 (9H, s), 1.61 (1H, q), 2.75 (2H, t), 2.93-2.97 (1H, m), 3.08-3.12 (1H, m), 3.30-3.33 (2H, m), 3.51 (2H, t), 4.36 (1H, t), 4.52 (2H, s), 6.73 (1H, d), 6.83 (1H, s), 7.80 (1H, d)

Step 7. Preparation of tert-butyl 5-{[(2R)-5-({4-[5-(methoxycarbonyl)-1-methyl-2-oxopyridin-3-yl]-2-methylpyrazol-3-yl}oxy)-2-methylpentyl]amino}-6-nitro-3,4-dihydro-1H-isoquinoline-2-carboxylate (INT-A102-7). To a stirred mixture of tert-butyl 5-{[(2R)-5-hydroxy-2-methylpentyl]amino}-6-nitro-3,4-dihydro-1H-isoquinoline-2-carboxylate (INT-A 102-6, 800 mg, 2.03 mmol, 1 equiv) and methyl 5-(5-hydroxy-1-methylpyrazol-4-yl)-1-methyl-6-oxopyridine-3-carboxylate (M2, 535 mg, 2.03 mmol, 1.0 equiv) in toluene (30 mL) was added 2-(tributyl-1\hat{ }[5]-phosphanylidene)acetonitrile (2 g, 10.16 mmol, 5 equiv) at room temperature under nitrogen atmosphere. The resulting mixture was stirred for 2 h at 100 °C and then cooled to room temperature. The reaction was quenched with water (100 mL) and the mixture was extracted with ethyl acetate (3×100 mL). The combined organic layers were washed with brine (3×100 mL), dried over anhydrous Na₂SO₄, and concentrated under reduced pressure. The residue was purified by C18 column using water and acetonitrile as mobile phase. Fractions containing the desired compound were evaporated to give 700 mg of tert-butyl 5-{[(2R)-5-({4-[5-(methoxycarbonyl)-1-methyl-2-oxopyridin-3-yl]-2-methylpyrazol-3-yl}oxy)-2-methylpentyl]amino}-6-nitro-3,4-dihydro-1H-isoquinoline-2-carboxylate (INT-A102-7) as a yellow oil (52%). LCMS: m/z (ESI), [M+H]⁺=639.30. ¹H NMR (DMSO-d₆, 400 MHz) δ 0.87 (3H, d), 0.92-0.96 (1H, m), 1.27 (1H, d), 1.44 (11H, s), 2.72-2.77 (2H, m), 2.94-3.03 (1H, m), 3.09-3.16 (1H, m), 3.18 (1H, d), 3.50 (2H, s), 3.58 (3H, s), 3.66 (3H, s), 3.78 (3H, s), 3.88-3.92 (2H, t), 4.50 (2H, s), 6.72 (1H, d), 6.83 (1H, s), 7.78 (1H, d), 8.00 (1H, s), 8.11 (1H, d), 8.45 (1H, d).

Step 8. Preparation of tert-butyl 6-amino-5-{[(2R)-5-({4-[5-(methoxycarbonyl)-1-methyl-2-oxopyridin-3-yl]-2-methylpyrazol-3-yl}oxy)-2-methylpentyl]amino}-3,4-dihydro-1H-isoquinoline-2-carboxylate (INT-A102-8). To a mixture of tert-butyl 5-{[(2R)-5-({4-[5-(methoxycarbonyl)-1-methyl-2-oxopyridin-3-yl]-2-methylpyrazol-3-yl}oxy)-2-methylpentyl]amino}-6-nitro-3,4-dihydro-1H-isoquinoline-2-carboxylate (700 mg, 1.09 mmol, 1 equiv), Raney Ni (187 mg, 2.19 mmol, 2 equiv) in methanol (8 mL) was added NH₂NH₂—H₂O (109 mg, 2.19 mmol, 2 equiv). The mixture was stirred for 30 min at 0 °C under nitrogen atmosphere. The mixture was filtered and the filter cake was washed with methanol (3×40 mL). The resulting solution was concentrated under reduced pressure. The residue was purified by Prep-TLC eluted with dichloromethane/methanol (10/1) to give 400 mg tert-butyl 6-amino-5-{[(2R)-5-({4-[5-(methoxycarbonyl)-1-methyl-2-oxopyridin-3-yl]-2-methylpyrazol-3-yl}oxy)-2-methylpentyl]amino}-3,4-dihydro-1H-isoquinoline-2-carboxylate (INT-A102-8) as a brown solid (59%). LCMS: m/z (ESI), [M+H]⁺=609.30.

Step 9. Preparation of methyl 5-(5-{[(4R)-4-{[2-amino-7-(tert-butoxycarbonyl)-6H,8H,9H-imidazo[4,5-f]isoquinolin-1-yl]methyl}pentyl]oxy}-1-methylpyrazol-4-yl)-1-methyl-6-oxopyridine-3-carboxylate (INT-A102-9). A mixture of tert-butyl 6-amino-5-{[(2R)-5-({4-[5-(methoxycarbonyl)-1-methyl-2-oxopyridin-3-yl]-2-methylpyrazol-3-yl}oxy)-2-methylpentyl]amino}-3,4-dihydro-1H-isoquinoline-2-carboxylate (400 mg, 0.65 mmol, 1 equiv) and BrCN (104 mg, 0.98 mmol, 1.5 equiv) in ethanol (5 mL) was stirred for 2 h at room temperature under nitrogen atmosphere. The reaction was quenched with water (50 mL) and the mixture was extracted with dichloromethane (3×50 mL). The combined organic layers were washed with brine (3×50 mL), dried over anhydrous Na₂SO₄, and concentrated under reduced pressure. The residue was purified by Prep-TLC eluted with dichloromethane/methanol (12/1) to give 300 mg of methyl 5-(5-{[(4R)-4-{[2-amino-7-(tert-butoxycarbonyl)-6H,8H,9H-imidazo[4,5-f]isoquinolin-1-yl]methyl}pentyl]oxy}-1-methylpyrazol-4-yl)-1-methyl-6-oxopyridine-3-carboxylate (INT-A102-9) as a brown solid (72%). LCMS: m/z (EST), [M+H]⁺=634.30.

Step 10. Preparation of 5-(5-{[(4R)-4-{[2-amino-7-(tert-butoxycarbonyl)-6H,8H,9H-imidazo[4,5-f]isoquinolin-1-yl]methyl}pentyl]oxy}-1-methylpyrazol-4-yl)-1-methyl-6-oxopyridine-3-carboxylic acid (INT-A102-10). A mixture of methyl 5-(5-{[(4R)-4-{[2-amino-7-(tert-butoxycarbonyl)-6H,8H,9H-imidazo[4,5-f]isoquinolin-1-yl]methyl}pentyl]oxy}-1-methylpyrazol-4-yl)-1-methyl-6-oxopyridine-3-carboxylate (300 mg, 0.47 mmol, 1 equiv) and LiOH—H₂O (99 mg, 2.36 mmol, 5 equiv) in tetrahydrofuran/H₂O (2 mL/0.5 mL) was stirred for 2 h at room temperature under nitrogen atmosphere. The resulting mixture was concentrated under reduced pressure. The residue was purified by C18 column using water and acetonitrile as mobile phase. Fractions containing the desired compound were evaporated to dryness to give 250 mg of 5-(5-{[(4R)-4-{[2-amino-7-(tert-butoxycarbonyl)-6H,8H,9H-imidazo[4,5-f]isoquinolin-1-yl]methyl}pentyl]oxy}-1-methylpyrazol-4-yl)-1-methyl-6-oxopyridine-3-carboxylic acid (INT-A102-10) as a white solid (85%). LCMS: m/z (EST), [M+H]⁺=620.40.

Step 11. Preparation of tert-butyl (11R)-5,11,30-trimethyl-27,31-dioxo-7-oxa-4,5,13,18,24,26,30-heptaazahexacyclo[26.3.1.0\hat{ }{2,6}.0\hat{ }{13,25}.0\hat{ }{14,23}.0\hat{ }{15,20}]dotriaconta-1(32),2(6),3,14,20,22,24,28-octaene-18-carboxylate (INT-A102-11). A mixture of 5-(5-{[(4R)-4-{[2-amino-7-(tert-butoxycarbonyl)-6H,8H,9H-imidazo[4,5-f]isoquinolin-1-yl]methyl}pentyl]oxy}-1-methylpyrazol-4-yl)-1-methyl-6-oxopyridine-3-carboxylic acid (200 mg, 0.32 mmol, 1 equiv), N,N,N,N-tetramethyl-O-(7-azabenzotriazol-1-yl)uronium hexafluorophospate (245 mg, 0.64 mmol, 2 equiv), and N,N-diisopropylethylamine (125 mg, 0.96 mmol, 3 equiv) in 1,4-dioxane (5 mL) was stirred for 30 min at 60 °C under nitrogen atmosphere. The mixture was cooled to room temperature. The reaction was quenched with water (20 mL) and the mixture was extracted with dichloromethane (2×20 mL). The combined organic layers were washed with brine (3×30 mL), dried over anhydrous Na₂SO₄, and concentrated under reduced pressure. The residue was purified by Prep-TLC eluted with (dichloromethane/methanol 12/1) to give 180 mg of tert-butyl (11R)-5,11,30-trimethyl-27,31-dioxo-7-oxa-4,5,13,18,24,26,30-heptaazahexacyclo[26.3.1.0\hat{ }{2,6}.0\hat{ }{13,25}.0\hat{ }{14,23}.0\hat{ }{15,20}]dotriaconta-1(32),2(6),3,14,20,22,24,28-octaene-18-carboxylate (INT-A102-11) as a white solid (92%). LCMS: m/z (ESI), [M+H]⁺=602.30.

Step 12. Preparation of (11R)-5,11,30-trimethyl-7-oxa-4,5,13,18,24,26,30-heptaazahexacyclo[26.3.1.0\hat{ }{2,6}.0\hat{ }{13,25}.0\hat{ }{14,23}.0\hat{ }{15,20}]dotriaconta-1(32),2(6),3,14,20,22,24,28-octaene-27,31-dione (EXAMPLE A102). A mixture of tert-butyl (11R)-5,11,30-trimethyl-27,31-dioxo-7-oxa-4,5,13,18,24,26,30-heptaazahexacyclo[26.3.1.0\hat{ }{2,6}.0\hat{ }{13,25}.0\hat{ }{14,23}.0\hat{ }{15,20}]dotriaconta-1(32),2(6),3,14,20,22,24,28-octaene-18-carboxylate (200 mg, 0.33 mmol, 1 equiv) in dichloromethane/trifluoroacetic acid (3 mL/1 mL) was stirred for 30 min at room temperature under nitrogen atmosphere. The reaction mixture was concentrated under reduced pressure. The residue was treated with saturated NaHCO₃ (aq.) (50 mL) and extracted with dichloromethane (3×50 mL). The combined organic layers were washed with brine (3×50 mL), dried over anhydrous Na₂SO₄, and concentrated under reduced pressure. The residue was purified by Prep-TLC eluted with dichloromethane/methanol (10/1) to give 110 mg of (11R)-5,11,30-trimethyl-7-oxa-4,5,13,18,24,26,30-heptaazahexacyclo[26.3.1.0\hat{ }{2,6}.0\hat{ }{13,25}.0\hat{ }{14,23}.0\hat{ }{15,20}]dotriaconta-1(32),2(6),3,14,20,22,24,28-octaene-27,31-dione (EXAMPLE A102) as a white solid (65%). LCMS: m/z (EST), [M+H]⁺=502.45. ¹H NMR (DMSO-d₆, 400 MHz) δ 0.81 (3H, d), 1.21-1.27 (1H, m), 1.41-1.45 (1H, m), 1.76-2.00 (4H, m), 2.35 (3H, s), 2.60-2.76 (2H, m), 3.52-3.58 (2H, m), 3.62 (3H, s), 3.71 (3H, s), 3.85-3.93 (1H, m), 4.11-4.27 (1H, m), 4.27-4.37 (1H, m), 6.91 (1H, d), 7.38 (1H, d), 8.28 (1H, d), 8.36 (1H, s), 8.77 (1H, d), 12.62 (1H, s).

Step 13. Preparation of (11R)-5,11,18,30-tetramethyl-7-oxa-4,5,13,18,24,26,30-heptaazahexacyclo[26.3.1.0\hat{ }{2,6}.0\hat{ }{13,25}.0\hat{ }{14,23}.0\hat{ }{15,20}]dotriaconta-1(32),2(6),3,14,20,22,24,28-octaene-27,31-dione (EXAMPLE A103). To a stirred mixture of (11R)-5,11,30-trimethyl-7-oxa-4,5,13,18,24,26,30-heptaazahexacyclo[26.3.1.0\hat{ }{2,6}.0\hat{ }{13,25}.0\hat{ }{14,23}.0\hat{ }{15,20}]dotriaconta-1(32),2(6),3,14,20,22,24,28-octaene-27,31-dione (EXAMPLE A102, 50 mg, 0.10 mmol, 1 equiv), paraformaldehyde (26 mg, 0.30 mmol, 3 equiv) and N,N-diisopropylethylamine (25 mg, 0.20 mmol, 2 equiv) in dichloromethane (2 mL) was added NaBH(OAc)₃ (42 mg, 0.20 mmol, 2 equiv) at room temperature under nitrogen atmosphere. The reaction was quenched with water (50 mL) and the mixture was extracted with dichloromethane (3×50 mL). The combined organic layers were washed with brine (3×50 mL), dried over anhydrous Na₂SO₄, and concentrated under reduced pressure. The residue was purified by prep-HPLC with XBridge Prep OBD C18 column using water containing 10 mmol/L NH₄HCO₃ and acetonitrile as mobile phase. Fractions containing the desired compound were evaporated to dryness to give 26 mg of (11R)-5,11,18,30-tetramethyl-7-oxa-4,5,13,18,24,26,30-heptaazahexacyclo[26.3.1.0\hat{ }{2,6}.0\hat{ }{13,25}.0\hat{ }{14,23}.0\hat{ }{15,20}]dotriaconta-1(32),2(6),3,14,20,22,24,28-octaene-27,31-dione (EXAMPLE A103) as a yellow solid (50%). LCMS: m/z (ESI), [M+H]⁺=516.30 ¹H NMR (DMSO-d₆, 400 MHz) δ 0.81 (3H, d), 1.21-1.27 (1H, m), 1.41-1.45 (1H, m), 1.76-2.00 (3H, m), 2.35 (3H, s), 2.60-2.76 (2H, m), 3.21 (3H, s), 3.52-3.58 (2H, m), 3.62 (3H, s), 3.71 (3H, s), 3.85-3.93 (1H, m), 4.11-4.27 (1H, m), 4.27-4.37 (1H, m), 6.91 (1H, d), 7.38 (1H, d), 8.28 (1H, d), 8.36 (1H, s), 8.77 (1H, d), 12.62 (1H, s).

### Example A109

### (12R)-12,27-dimethyl-17-(4-methylpiperazin-1-yl)-8-oxa-14,21,23,27-tetraazapentacyclo[23.3.1.0\hat{ }{2,7}.0\hat{ }{14,22}.0\hat{ }{15,20}]nonacosa-1(29),2(7),3,5,15,17,19,21,25-nonaene-24,28-dione

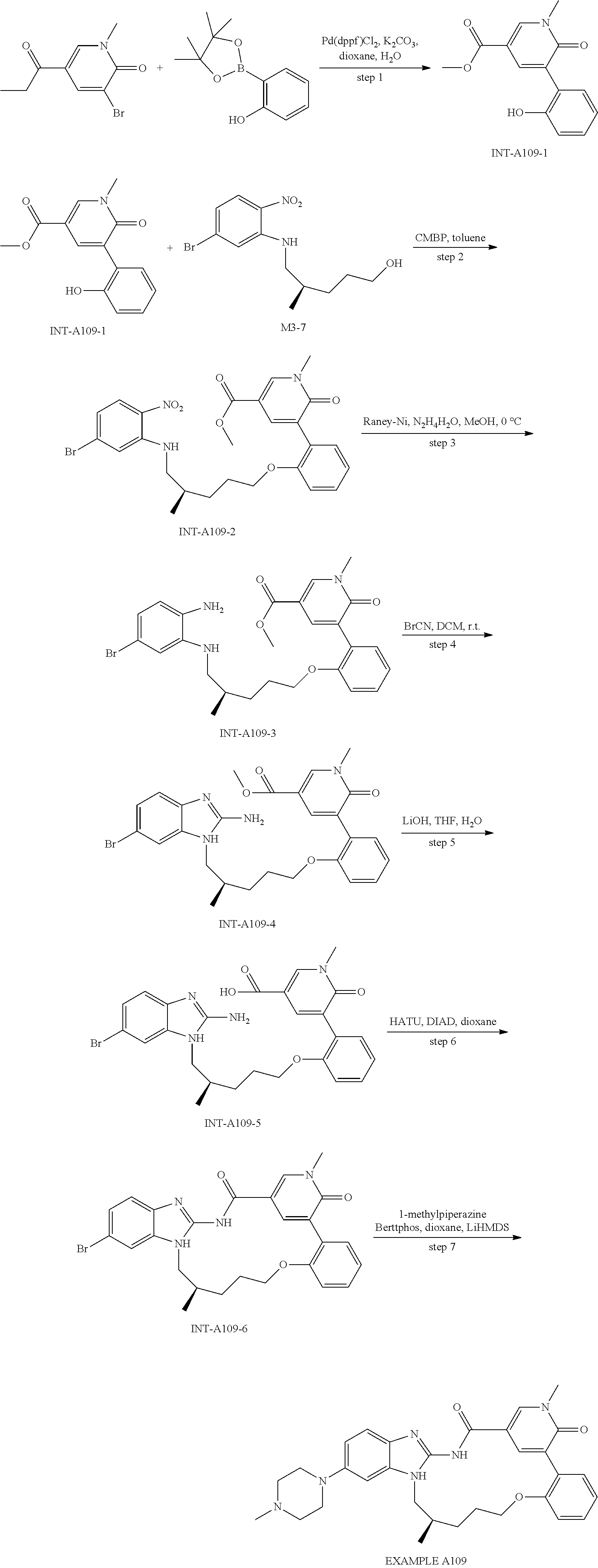

Step 1. Preparation of methyl 5-(2-hydroxyphenyl)-1-methyl-6-oxopyridine-3-carboxylate (INT-A109-1). A mixture of methyl 5-bromo-1-methyl-6-oxopyridine-3-carboxylate (8 g, 32.51 mmol, 1 equiv), 2-(4,4,5,5-tetramethyl-1,3,2-dioxaborolan-2-yl)phenol (7.87 g, 35.76 mmol, 1.1 equiv), Pd(dppf)Cl₂ (1.43 g, 1.95 mmol, 0.06 equiv), and K₂CO₃ (8.99 g, 65.02 mmol, 2 equiv) in 1,4-dioxane (240 mL) and H₂O (60 mL) was stirred for 1.5 h at 80 °C under nitrogen atmosphere. The mixture was cooled to room temperature. The reaction was quenched with water (500 mL) and the mixture was extracted with CH₂Cl₂ (3×500 mL). The combined organic layers were washed with brine (3×500 mL), dried over anhydrous Na₂SO₄, and concentrated under reduced pressure. The residue was purified by prep-TLC eluted with petroleum ether/ethyl acetate (3/1) to give 5.6 g of methyl 5-(2-hydroxyphenyl)-1-methyl-6-oxopyridine-3-carboxylate (INT-A109-1) as a brown solid (66%). LCMS: m/z (EST), [M+H]⁺=260.05. ¹H NMR (DMSO-d₆, 400 MHz) δ 3.61 (3H, s), 3.82 (3H, s), 6.81-6.93 (2H, m), 7.18-7.22 (1H, m), 7.24-7.28 (1H, m), 7.86 (1H, d), 8.60 (1H, d), 9.39 (1H, s).

Step 2. Preparation of methyl 5-(2-{[(4R)-5-[(5-bromo-2-nitrophenyl)amino]-4-methylpentyl]oxy}phenyl)-1-methyl-6-oxopyridine-3-carboxylate (INT-A109-2). A mixture of methyl 5-(2-hydroxyphenyl)-1-methyl-6-oxopyridine-3-carboxylate (1 g, 3.86 mmol, 1 equiv), 2-(tributyl-1\hat{ }[5]-phosphanylidene)acetonitrile (4.65 g, 19.29 mmol, 5 equiv), and (4R)-5-[(5-bromo-2-nitrophenyl)amino]-4-methylpentan-1-ol (M3-7, 1.22 g, 3.86 mmol, 1 equiv) in toluene (40 mL) was stirred for 1.5 h at 130 °C under nitrogen atmosphere. The mixture was cooled to room temperature. The reaction was quenched with water (200 mL) and the mixture was extracted with ethyl acetate (3×200 mL). The combined organic layers were washed with brine (3×200 mL), dried over anhydrous Na₂SO₄, and concentrated under reduced pressure. The residue was purified by prep-TLC eluted with petroleum ether/ethyl acetate (3/1) to give 1.7 g of methyl 5-(2-{[(4R)-5-[(5-bromo-2-nitrophenyl)amino]-4-methylpentyl]oxy}phenyl)-1-methyl-6-oxopyridine-3-carboxylate (INT-A109-2) as a reddish solid (79%). LCMS: m/z (ESI), [M+H]⁺=558.10.

Step 3. Preparation of methyl 5-(2-{[(4R)-5-[(2-amino-5-bromophenyl)amino]-4-methylpentyl]oxy}phenyl)-1-methyl-6-oxopyridine-3-carboxylate (INT-A109-3).

To a stirred mixture of methyl 5-(2-{[(4R)-5-[(5-bromo-2-nitrophenyl)amino]-4-methylpentyl]oxy}phenyl)-1-methyl-6-oxopyridine-3-carboxylate (800 mg, 1.43 mmol, 1 equiv) and Raney-Ni (80 mg, 0.93 mmol, 0.65 equiv) in methanol (35 mL) was added NH₂NH₂—H₂O (143 mg, 2.87 mmol, 2 equiv) at 0 °C. The resulting mixture was stirred for 2 h at room temperature. The reaction was quenched with water (100 mL) and the mixture was extracted with ethyl acetate (3×80 mL). The combined organic layers were washed with brine (3×80 mL), dried over anhydrous Na₂SO₄, and concentrated under reduced pressure. The residue was purified by Prep-TLC eluted with petroleum ether/ethyl acetate (1/5) to give 690 mg of methyl 5-(2-{[(4R)-5-[(2-amino-5-bromophenyl)amino]-4-methylpentyl]oxy}phenyl)-1-methyl-6-oxopyridine-3-carboxylate (INT-A109-3) as a reddish solid (91%). LCMS: m/z (EST), [M+H]⁺=528.00.

Step 4. Preparation of methyl 5-(2-{[(4R)-4-[(2-amino-6-bromo-1,3-benzodiazol-1-yl)methyl]pentyl]oxy}phenyl)-1-methyl-6-oxopyridine-3-carboxylate (INT-A109-4). A mixture of methyl 5-(2-{[(4R)-5-[(2-amino-5-bromophenyl)amino]-4-methylpentyl]oxy}phenyl)-1-methyl-6-oxopyridine-3-carboxylate (1.3 g, 2.46 mmol, 1 equiv) and BrCN (521 mg, 4.92 mmol, 2 equiv) in dichloromethane (30 mL) was stirred for 4 h at room temperature. The reaction was quenched with water (200 mL) and the mixture was extracted with dichloromethane (3×200 mL). The combined organic layers were washed with brine (3×200 mL), dried over anhydrous Na₂SO₄, and concentrated under reduced pressure. The residue was purified by Prep-TLC eluted with dichloromethane/methanol (20/1) to give 1.32 g of methyl 5-(2-{[(4R)-4-[(2-amino-6-bromo-1,3-benzodiazol-1-yl)methyl]pentyl]oxy}phenyl)-1-methyl-6-oxopyridine-3-carboxylate (INT-A109-4) as a reddish solid (97%). LCMS: m/z (EST), [M+H]⁺=554.95.

Step 5. Preparation of 5-(2-{[(4R)-4-[(2-amino-6-bromo-1,3-benzodiazol-1-yl)methyl]pentyl]oxy}phenyl)-1-methyl-6-oxopyridine-3-carboxylic acid (INT-A109-5). To a stirred mixture of methyl 5-(2-{[(4R)-4-[(2-amino-6-bromo-1,3-benzodiazol-1-yl)methyl]pentyl]oxy}phenyl)-1-methyl-6-oxopyridine-3-carboxylate (1.3 g, 2.35 mmol, 1 equiv) in tetrahydrofuran (10 mL) was added LiOH (225.02 mg, 9.39 mmol, 4 equiv) and H₂O (4 mL) at room temperature. The resulting mixture was stirred for 1 h at room temperature and concentrated under vacuum. The residue was purified by C18 flash chromatography using water and acetonitrile as mobile phase. Fractions containing the desired compound were evaporated to dryness to give 900 mg of 5-(2-{[(4R)-4-[(2-amino-6-bromo-1,3-benzodiazol-1-yl)methyl]pentyl]oxy}phenyl)-1-methyl-6-oxopyridine-3-carboxylic acid (INT-A109-5) as a red solid (710%). LCMS: m/z (EST), [M+H]⁺=540.95.

Step 6. Preparation of (12R)-17-bromo-12,27-dimethyl-8-oxa-14,21,23,27-tetraazapentacyclo[23.3.1.0\hat{ }{2,7}.0\hat{ }{14,22}.0\hat{ }{15,20}]nonacosa-1(29),2(7),3,5,15,17,19,21,25-nonaene-24,28-dione (INT-A109-6). To a stirred mixture of 5-(2-{[(4R)-4-[(2-amino-6-bromo-1,3-benzodiazol-1-yl)methyl]pentyl]oxy}phenyl)-1-methyl-6-oxopyridine-3-carboxylic acid (450 mg, 0.83 mmol, 1 equiv) in dioxane (10 mL) were added N,N-diisopropylethylamine (323 mg, 2.50 mmol, 3 equiv) and N,N,N,N-tetramethyl-O-(7-azabenzotriazol-1-yl)uronium hexafluorophospate (476 mg, 1.25 mmol, 1.5 equiv) at room temperature. The resulting mixture was stirred for 1 h at 60 °C under nitrogen atmosphere and cooled to room temperature. The reaction was quenched with water (100 mL) and the mixture was extracted with ethyl acetate (3×100 mL). The combined organic layers were washed with brine (3×100 mL), dried over anhydrous Na₂SO₄, and concentrated under reduced pressure. The residue was purified by prep-TLC eluted with methylene dichloromethane/methanol (30/1) to give 350 mg of (12R)-17-bromo-12,27-dimethyl-8-oxa-14,21,23,27-tetraazapentacyclo[23.3.1.0\hat{ }{2,7}.0\hat{ }{14,22}.0\hat{ }{15,20}]nonacosa-1(29),2(7),3,5,15,17,19,21,25-nonaene-24,28-dione (INT-A109-6) as a reddish solid (80%). LCMS: m/z (EST), [M+H]⁺=523.05.

Step 7. Preparation of (12R)-12,27-dimethyl-17-(4-methylpiperazin-1-yl)-8-oxa-14,21,23,27-tetraazapentacyclo[23.3.1.0\hat{ }{2,7}.0\hat{ }{14,22}.0\hat{ }{15,20}]nonacosa-1(29),2(7),3,5,15,17,19,21,25-nonaene-24,28-dione (Example A109). To a stirred mixture of (12R)-17-bromo-12,27-dimethyl-8-oxa-14,21,23,27-tetraazapentacyclo[23.3.1.0\hat{ }{2,7}.0\hat{ }{14,22}.0\hat{ }{15,20}]nonacosa-1(29),2(7),3,5,15,17,19,21,25-nonaene-24,28-dione (200 mg, 0.38 mmol, 1 equiv), 1-methylpiperazine (127 mg, 1.27 mmol, 3.31 equiv), and BrettPhos Pd G3 (90 mg, 0.10 mmol, 0.26 equiv) in 1,4-dioxane (10 mL) was added LiHMDS (3.8 mL, 3.8 mmol, 10 equiv) dropwise at room temperature under nitrogen atmosphere. The resulting mixture was stirred for 50 min at 60 °C and cooled to room temperature. The reaction was quenched with sat. NH₄Cl (aq.) (100 mL) and the mixture was extracted with dichloromethane (3×100 mL). The combined organic layers were washed with brine (3×100 mL), dried over anhydrous Na₂SO₄, and concentrated under reduced pressure. The residue was purified by prep-TLC eluted with dichloromethane/methanol (30/1) and the product was further purified by prep-HPLC with XBridge Prep OBD C18 column using water containing 10 mmol/L NH₄HCO₃ and acetonitrile as mobile phase. Fractions containing the desired compound were evaporated to dryness to give 13.7 mg of (12R)-12,27-dimethyl-17-(4-methylpiperazin-1-yl)-8-oxa-14,21,23,27-tetraazapentacyclo[23.3.1.0\hat{ }{2,7}.0\hat{ }{14,22}.0\hat{ }{15,20}]nonacosa-1(29),2(7),3,5,15,17,19,21,25-nonaene-24,28-dione (Example A109) an off-white solid (7%). LCMS: m/z (EST), [M+H]⁺=541.35. ¹H NMR (DMSO-d₆, 400 MHz) δ 0.92 (3H, d), 1.29-1.33 (1H, m), 1.60-1.66 (1H, m), 2.02-2.06 (2H, m), 2.24 (3H, s), 2.26-2.30 (1H, m), 2.49-2.51 (4H, m), 3.14 (4H, t), 3.60 (3H, s), 3.93-4.08 (3H, m), 4.20 (1H, t), 6.83-6.89 (1H, m), 6.86 (1H, dd), 6.92-6.96 (1H, m), 7.12 (1H, d), 7.25-7.29 (1H, m), 7.34 (1H, d), 7.69 (1H, dd), 8.33 (1H, d), 8.85 (1H, d), 12.27 (1H, s).

### Example A110 (12R)-4-fluoro-12,27-dimethyl-17-(4-methylpiperazin-1-yl)-8-oxa-14,21,23,27-tetraazapentacyclo[23.3.1.0\hat{ }{2,7}.0\hat{ }{14,22}.0\hat{ }{15,20}]nonacosa-1(29),2(7),3,5,15,17,19,21,25-nonaene-24,28-dione

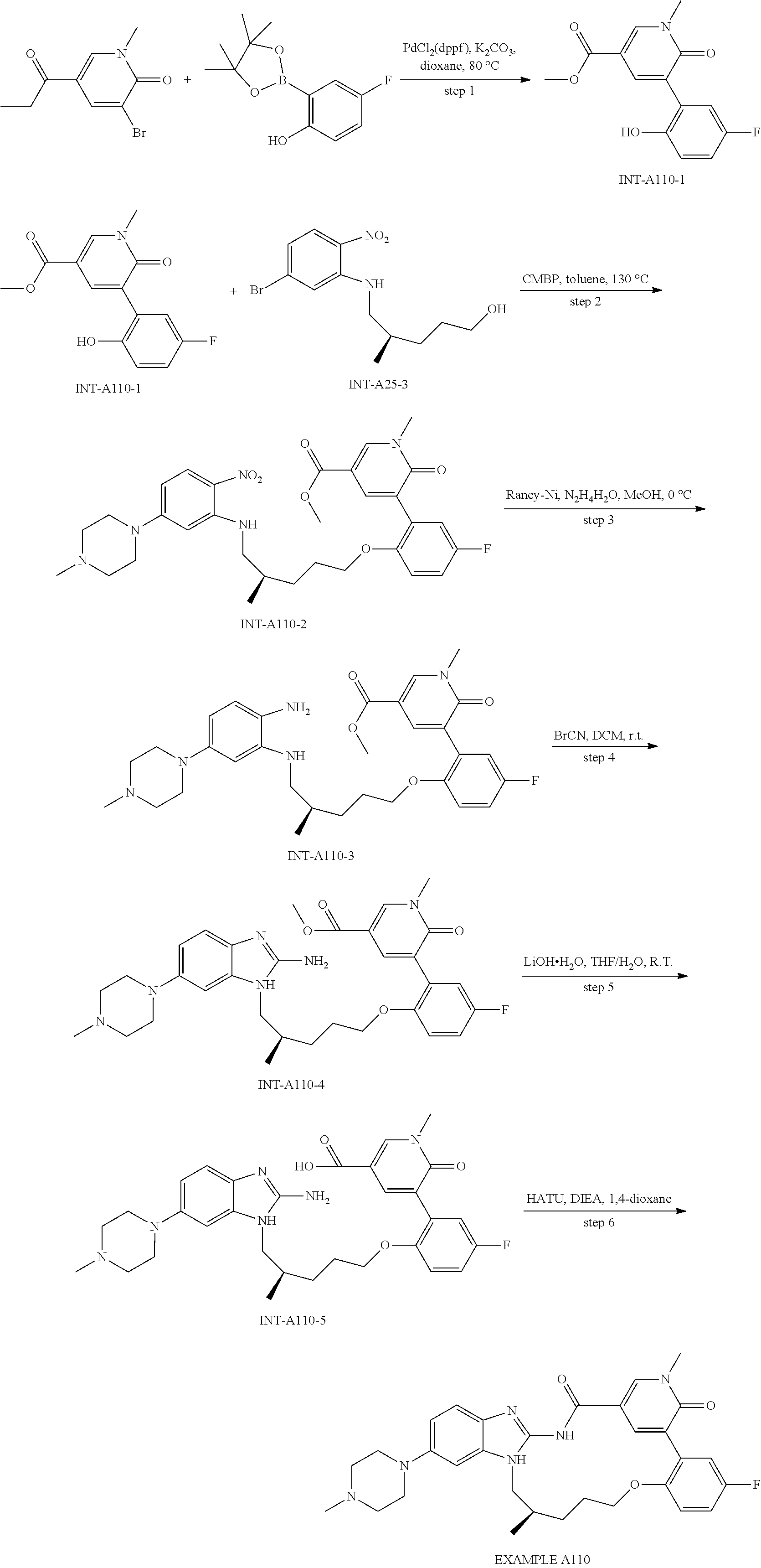

Step 1. Preparation of methyl 5-(5-fluoro-2-hydroxyphenyl)-1-methyl-6-oxopyridine-3-carboxylate (INT-A110-1). A mixture of methyl 5-bromo-1-methyl-6-oxopyridine-3-carboxylate (2 g, 8.128 mmol, 1 equiv), 4-fluoro-2-(4,4,5,5-tetramethyl-1,3,2-dioxaborolan-2-yl)phenol (2.32 g, 9.754 mmol, 1.2 equiv), Pd(dppf)Cl₂ (0.48 g, 0.656 mmol, 0.08 equiv), and K₂CO₃ (2.81 g, 20.320 mmol, 2.5 equiv) in dioxane (40 mL) and H₂O (10 mL) was stirred for 2 h at 80 °C under nitrogen atmosphere. The mixture was cooled to room temperature. The reaction was quenched with water (150 mL) and the mixture was extracted with dichloromethane (3×100 mL). The combined organic layers were washed with brine (3×100 mL), dried over anhydrous Na₂SO₄, and concentrated under reduced pressure. The residue was purified by silica gel chromatography eluted with dichloromethane/methanol (15/1) to give 1 g of methyl 5-(5-fluoro-2-hydroxyphenyl)-1-methyl-6-oxopyridine-3-carboxylate (INT-A110-1) as a yellow solid (44%). ¹H NMR (DMSO-d₆, 400 MHz) 3.60 (3H, s), 3.81 (3H, s), 6.84-6.95 (1H, m), 6.96-7.08 (1H, m), 7.14 (1H, d), 7.94 (1H, d), 8.61 (1H, d), 9.41 (1H, s)

Step 2. methyl 5-(5-fluoro-2-{[(4R)-4-methyl-5-{[5-(4-methylpiperazin-1-yl)-2-nitrophenyl]amino}pentyl]oxy}phenyl)-1-methyl-6-oxopyridine-3-carboxylate (INT-A110-2). A mixture of methyl 5-(5-fluoro-2-hydroxyphenyl)-1-methyl-6-oxopyridine-3-carboxylate (526 mg, 1.90 mmol, 1 equiv), (4R)-4-methyl-5-{[5-(4-methylpiperazin-1-yl)-2-nitrophenyl]amino}pentan-1-ol (639.07 mg, 1.90 mmol, 1 equiv), and 2-(tributyl-1\hat{ }[5]-phosphanylidene)acetonitrile (2.30 g, 9.50 mmol, 5 equiv) in toluene (5 mL) was stirred overnight at 130 °C under nitrogen atmosphere. The mixture was cooled to room temperature. The reaction was quenched with water (100 mL) and the mixture was extracted with dichloromethane (3×100 mL). The combined organic layers were washed with brine (3×100 mL), dried over anhydrous Na₂SO₄, and concentrated under reduced pressure. The residue was purified by Prep-TLC eluted with petroleum ether/ethyl acetate (1/1) to give 500 mg of methyl 5-(5-fluoro-2-{[(4R)-4-methyl-5-{[5-(4-methylpiperazin-1-yl)-2-nitrophenyl]amino}pentyl]oxy}phenyl)-1-methyl-6-oxopyridine-3-carboxylate (INT-A110-2) as a white solid (44%). ¹H NMR (DMSO-d₆, 400 MHz) 0.90 (3H, s), 1.31-1.39 (4H, m), 2.20 (3H, s), 2.32-2.41 (4H, m), 2.44 (1H, t), 2.98-3.28 (2H, m), 3.40 (4H, t), 3.56 (3H, s), 3.76 (3H, s), 3.95 (2H, t), 5.77 (1H, s), 5.96 (1H, d), 6.43 (1H, d), 6.97-7.29 (4H, m), 7.83-7.95 (2H, m), 8.05 (1H, d), 8.39 (1H, t), 8.53 (1H, d).

Step 3. Preparation of methyl 5-(2-{[(4R)-5-{[2-amino-5-(4-methylpiperazin-1-yl)phenyl]amino}-4-methylpentyl]oxy}-5-fluorophenyl)-1-methyl-6-oxopyridine-3-carboxylate (INT-A110-3). To a stirred mixture of methyl 5-(5-fluoro-2-{[(4R)-4-methyl-5-{[5-(4-methylpiperazin-1-yl)-2-nitrophenyl]amino}pentyl]oxy}phenyl)-1-methyl-6-oxopyridine-3-carboxylate (500 mg, 0.83 mmol, 1 equiv) and Raney Nickel (3.60 mg, 0.042 mmol, 0.05 equiv) in methanol (20 mL) was added hydrazine hydrate (126.06 mg, 2.51 mmol, 3 equiv) at 0 °C. The mixture was stirred for 30 min at 0 °C under nitrogen atmosphere. The resulting mixture was filtered and the filter cake was washed with methanol (3×30 mL). The resulting solution was concentrated under reduced pressure. The residue was purified by C18 flash chromatography using water and acetonitrile as mobile phase. Fractions containing the desired compound were evaporated to dryness to give 400 mg of methyl 5-(2-{[(4R)-5-{[2-amino-5-(4-methylpiperazin-1-yl)phenyl]amino}-4-methylpentyl]oxy}-5-fluorophenyl)-1-methyl-6-oxopyridine-3-carboxylate (INT-A110-3) as a yellow solid (84%). LCMS: m/z (ESI), [M+H]⁺=566.15.

Step 4. Preparation of methyl 5-(2-{[(4R)-4-{[2-amino-6-(4-methylpiperazin-1-yl)-1,3-benzodiazol-1-yl]methyl}pentyl]oxy}-5-fluorophenyl)-1-methyl-6-oxopyridine-3-carboxylate (INT-A110-4). A mixture of methyl 5-(2-{[(4R)-5-{[2-amino-5-(4-methylpiperazin-1-yl)phenyl]amino}-4-methylpentyl]oxy}-5-fluorophenyl)-1-methyl-6-oxopyridine-3-carboxylate (240 mg, 0.42 mmol, 1 equiv) and BrCN (51 mg, 0.46 mmol, 1.1 equiv) in dichloromethane (5 mL) was stirred for 4 h at room temperature under nitrogen atmosphere and concentrated under reduced pressure. The residue was purified by Prep-TLC eluted with dichloromethane/methanol (1/1) to give 140 mg of methyl 5-(2-{[(4R)-4-{[2-amino-6-(4-methylpiperazin-1-yl)-1,3-benzodiazol-1-yl]methyl}pentyl]oxy}-5-fluorophenyl)-1-methyl-6-oxopyridine-3-carboxylate (INT-A110-4) as a yellow solid (55%). LCMS: m/z (ESI), [M+H]⁺=591.15.

Step 5. Preparation of 5-(2-{[(4R)-4-{[2-amino-6-(4-methylpiperazin-1-yl)-1,3-benzodiazol-1-yl]methyl}pentyl]oxy}-5-fluorophenyl)-1-methyl-6-oxopyridine-3-carboxylic acid (INT-A110-5). A mixture of methyl 5-(2-{[(4R)-4-{[2-amino-6-(4-methylpiperazin-1-yl)-1,3-benzodiazol-1-yl]methyl}pentyl]oxy}-5-fluorophenyl)-1-methyl-6-oxopyridine-3-carboxylate (75 mg, 0.12 mmol, 1 equiv) and LiOH·H₂O (10 mg, 0.25 mmol, 2 equiv) in tetrahydrofuran (2 mL) and water (0.5 mL) was stirred for 2 h at room temperature under nitrogen atmosphere. The reaction mixture was concentrated under reduced pressure. The residue was purified by C18 flash chromatography using water and acetonitrile as mobile phase. Fractions containing the desired compound were evaporated to dryness to give 40 mg of 5-(2-{[(4R)-4-{[2-amino-6-(4-methylpiperazin-1-yl)-1,3-benzodiazol-1-yl]methyl}pentyl]oxy}-5-fluorophenyl)-1-methyl-6-oxopyridine-3-carboxylic acid (INT-A 110-5) as a yellow oil (54%) LCMS: m/z (ESI), [M+H]⁺=577.25.

Step 6. Preparation of (12R)-4-fluoro-12,27-dimethyl-17-(4-methylpiperazin-1-yl)-8-oxa-14,21,23,27-tetraazapentacyclo[23.3.1.0\hat{ }{2,7}.0\hat{ }{14,22}.0\hat{ }{15,20}]nonacosa-1(29),2(7),3,5,15,17,19,21,25-nonaene-24,28-dione (EXAMPLE A110). A mixture of 5-(2-{[(4R)-4-{[2-amino-6-(4-methylpiperazin-1-yl)-1,3-benzodiazol-1-yl]methyl}pentyl]oxy}-5-fluorophenyl)-1-methyl-6-oxopyridine-3-carboxylic acid (35 mg, 0.06 mmol, 1 equiv), N,N,N,N-tetramethyl-O-(7-azabenzotriazol-1-yl)uronium hexafluorophospate (34 mg, 0.09 mmol, 1.5 equiv), and N,N-diisopropylethylamine (23 mg, 0.18 mmol, 3 equiv) in 1,4-dioxane (2 mL) was stirred for 2 h at room temperature under nitrogen atmosphere. The reaction was quenched with water (50 mL) and the mixture was extracted with dichloromethane (3×50 mL). The combined organic layers were washed with brine (3×50 mL), dried over anhydrous Na₂SO₄, and concentrated under reduced pressure. The residue was purified by prep-HPLC with XBridge Shield RP18 OBD column using water containing 10 mmol/L NH₄HCO₃ and 0.1% NH₃·H₂O and acetonitrile as mobile phase. Fractions containing the desired compound were evaporated to dryness to give 3.6 mg of (12R)-4-fluoro-12,27-dimethyl-17-(4-methylpiperazin-1-yl)-8-oxa-14,21,23,27-tetraazapentacyclo[23.3.1.0\hat{ }{2,7}.0\hat{ }{14,22}.0\hat{ }{15,20}]nonacosa-1(29),2(7),3,5,15,17,19,21,25-nonaene-24,28-dione (EXAMPLE A110) as a yellow green solid (10%). LCMS: m/z (ESI), [M+H]⁺=559.40. ¹H NMR (DMSO-d₆, 400 MHz) δ 0.94 (3H, d), 1.31-1.40 (1H, m), 1.64-1.70 (1H, m), 2.01-2.14 (2H, m), 2.21-2.30 (1H, m), 2.41 (3H, s), 2.66-2.73 (4H, m), 3.22 (4H, t), 3.60 (3H, s), 3.76-3.85 (1H, m), 3.99-4.09 (2H, m), 4.13 (1H, s), 6.87-6.97 (2H, m), 6.98-7.09 (2H, m), 7.24 (1H, d), 7.47-7.55 (1H, m), 8.23 (1H, s), 8.91 (1H, s).

### Example A111

### (12R)-4,5-difluoro-12,27-dimethyl-17-(4-methylpiperazin-1-yl)-8-oxa-14,21,23,27-tetraazapentacyclo[23.3.1.0\hat{ }{2,7}.0\hat{ }{14,22}.0\hat{ }{15,20}]nonacosa-1(29),2(7),3,5,15,17,19,21,25-nonaene-24,28-dione

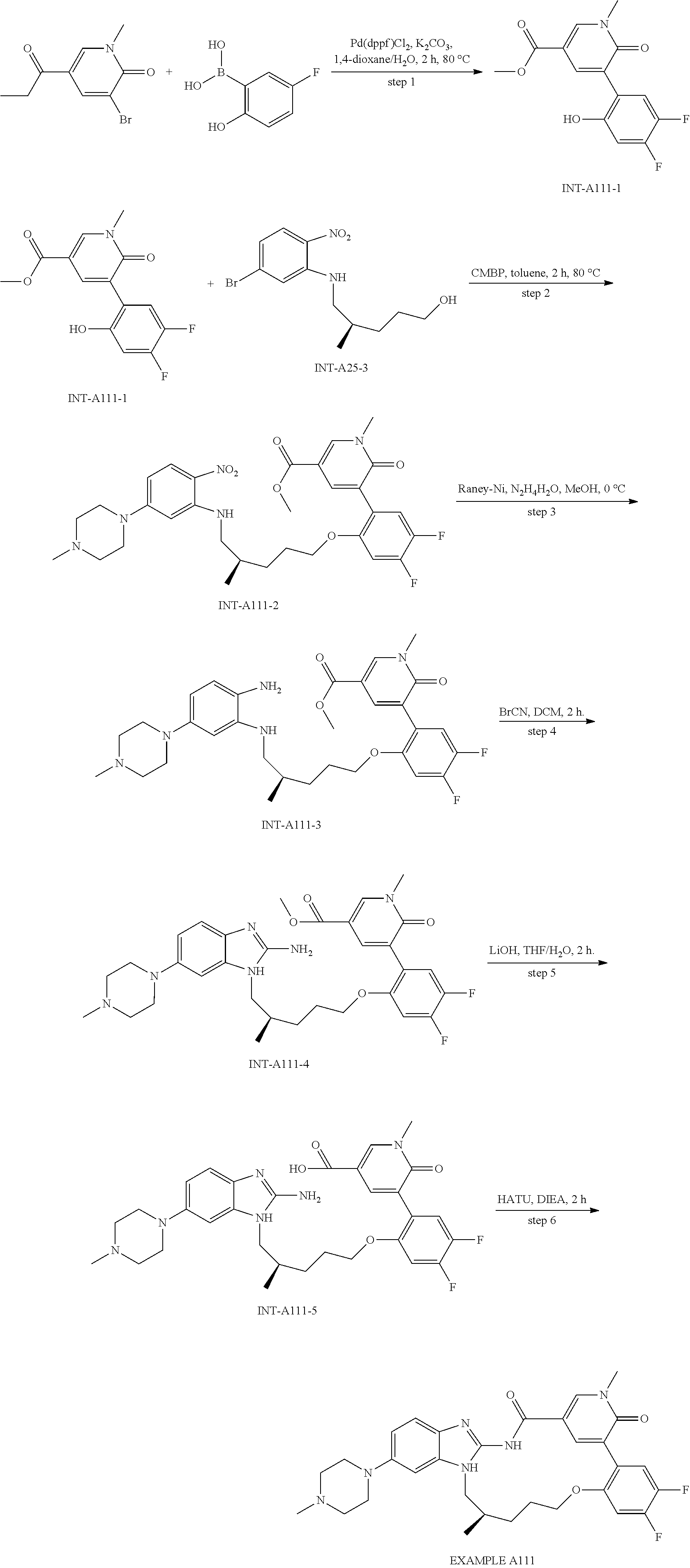

Step 1. Preparation of methyl 5-(4,5-difluoro-2-hydroxyphenyl)-1-methyl-6-oxopyridine-3-carboxylate (INT-A111-1). To a stirred mixture of methyl 5-bromo-1-methyl-6-oxopyridine-3-carboxylate (2.00 g, 8.12 mmol, 1 equiv) and 4,5-difluoro-2-hydroxyphenylboronic acid (2.12 g, 12.19 mmol, 1.5 equiv) in 1,4-dioxane (32 mL) and water (8 mL) were added 1,1′-bis(diphenylphosphino)ferrocene-palladium(II) (1.19 g, 1.62 mmol, 0.2 equiv) and K₂CO₃ (2.81 g, 20.32 mmol, 2.5 equiv) in portions at room temperature under nitrogen atmosphere. The resulting mixture was stirred for 2 h at 80 °C. The mixture was cooled to room temperature. The reaction was quenched with water (100 mL) and the mixture was extracted with dichloromethane (3×80 mL). The combined organic layers were washed with brine (3×50 mL), dried over anhydrous Na₂SO₄, and concentrated under reduced pressure. The residue was purified by C18 flash chromatography using water containg 10 mmol/L NH₄HCO₃ and 0.10% NH₃·H₂O and acetonitrile as mobile phase. Fractions containing the desired compound were evaporated to dryness to give 1.38 g of methyl 5-(4,5-difluoro-2-hydroxyphenyl)-1-methyl-6-oxopyridine-3-carboxylate (INT-A111-1) as a white solid (57%). ¹H NMR (DMSO-d_{d}, 400 MHz) δ 3.59 (3H, s), 3.81 (3H, s), 6.81-6.91 (1H, m), 7.33-7.41 (1H, d), 7.92 (1H, d), 8.61 (1H, d), 9.90 (1H, s).

Step 2. Preparation of methyl 5-(4,5-difluoro-2-{[(4R)-4-methyl-5-{[5-(4-methylpiperazin-1-yl)-2-nitrophenyl]amino}pentyl]oxy}phenyl)-1-methyl-6-oxopyridine-3-carboxylate (INT-A111-2). To a stirred mixture of methyl 5-(4,5-difluoro-2-hydroxyphenyl)-1-methyl-6-oxopyridine-3-carboxylate (500 mg, 1.69 mmol, 1 equiv) and (4R)-4-methyl-5-{[5-(4-methylpiperazin-1-yl)-2-nitrophenyl]amino}pentan-1-ol (INT-A25-3, 569 mg, 1.69 mmol, 1 equiv) in toluene (7 mL) was added 2-(tributyl-lambda5-phosphanylidene)acetonitrile (2.04 g, 8.47 mmol, 5 equiv) in portions at room temperature under nitrogen atmosphere. The resulting mixture was stirred for 2 h at 80 °C. The mixture was cooled to room temperature. The reaction was quenched with water (200 mL) and the mixture was extracted with dichloromethane (3×300 mL). The combined organic layers were washed with brine (3×200 mL), dried over anhydrous Na₂SO₄, and concentrated under reduced pressure. The residue was purified by C18 FLASH using water containing 10 mmol/L NH₄HCO₃ and 0.1% NH₃—H₂O and acetonitrile as mobile phase. Fractions containing the desired compound were evaporated to dryness to give 620 mg of methyl 5-(4,5-difluoro-2-{[(4R)-4-methyl-5-{[5-(4-methylpiperazin-1-yl)-2-nitrophenyl]amino}pentyl]oxy}phenyl)-1-methyl-6-oxopyridine-3-carboxylate (INT-A111-2) as a yellow oil (59%). LCMS: m/z (ESI), [M+H]⁺=614.25. ¹H NMR (DMSO-d₆, 400 MHz) δ 0.91 (4H, d), 1.20-1.32 (1H, m), 1.59-1.72 (3H, m), 1.75-1.87 (1H, m), 2.32 (3H, d), 3.04-3.13 (1H, m), 3.19-3.25 (1H, m), 3.320-3.36 (8H, m), 3.56 (3H, s), 3.76 (3H, s), 3.97 (3H, t), 5.76 (1H, s), 5.98 (1H, s), 6.40-6.46 (1H, m), 7.18-7.28 (1H, m), 7.40-7.50 (1H, m), 7.84-7.95 (2H, m), 8.37 (1H, t), 8.53 (1H, d).

Step 3. Preparation of methyl 5-(2-{[(4R)-5-{[2-amino-5-(4-methylpiperazin-1-yl)phenyl]amino}-4-methylpentyl]oxy}-4,5-difluorophenyl)-1-methyl-6-oxopyridine-3-carboxylate (INT-A111-3). To a stirred mixture of methyl 5-(4,5-difluoro-2-{[(4R)-4-methyl-5-{[5-(4-methylpiperazin-1-yl)-2-nitrophenyl]amino}pentyl]oxy}phenyl)-1-methyl-6-oxopyridine-3-carboxylate (600 mg, 0.97 mmol, 1 equiv) and Raney Ni (251 mg, 2.93 mmol, 3 equiv) in methanol (5 mL) was added hydrazine hydrate (98%) (2.45 mg, 0.04 mmol, 3 equiv) at room temperature under nitrogen atmosphere. The resulting mixture was stirred for 30 min at 0 °C under nitrogen atmosphere. The mixture was filtered and the filter cake was washed with methanol (3×10 mL). The resulting solution was concentrated under reduced pressure to give 310 mg of methyl 5-(2-{[(4R)-5-{[2-amino-5-(4-methylpiperazin-1-yl)phenyl]amino}-4-methylpentyl]oxy}-4,5-difluorophenyl)-1-methyl-6-oxopyridine-3-carboxylate (INT-A111-3) as a white solid. This material was used for the next reaction without further purification. LCMS: m/z (ESI), [M+H]⁺=582.25.

Step 4. Preparation of methyl 5-(2-{[(4R)-4-{[2-amino-6-(4-methylpiperazin-1-yl)-1,3-benzodiazol-1-yl]methyl}pentyl]oxy}-4,5-difluorophenyl)-1-methyl-6-oxopyridine-3-carboxylate (INT-A111-4). A mixture of methyl 5-(2-{[(4R)-5-{[2-amino-5-(4-methylpiperazin-1-yl)phenyl]amino}-4-methylpentyl]oxy}-4,5-difluorophenyl)-1-methyl-6-oxopyridine-3-carboxylate (700 mg, 1.20 mmol, 1 equiv) and cyanogen bromide (190 mg, 1.80 mmol, 1.5 equiv) in dichloromethane (10 mL) was stirred for 2 h at room temperature under nitrogen atmosphere. The reaction was quenched with water (10 mL) and the mixture was extracted with dichloromethane (3×60 mL). The combined organic layers were washed with brine (3×20 mL), dried over anhydrous Na₂SO₄, and concentrated under reduced pressure. The residue was purified by C18 FLASH using water containing 10 mmol/L NH₄HCO₃ and 0.1% NH₃·H₂O and acetonitrile as mobile phase. Fractions containing the desired compound were evaporated to dryness to give 120 mg of methyl 5-(2-{[(4R)-4-{[2-amino-6-(4-methylpiperazin-1-yl)-1,3-benzodiazol-1-yl]methyl}pentyl]oxy}-4,5-difluorophenyl)-1-methyl-6-oxopyridine-3-carboxylate (INT-A111-4) as a white solid (16%). LCMS: m/z (ESI), [M+H]⁺=609.15.

Step 5. Preparation of 5-(2-{[(4R)-4-{[2-amino-6-(4-methylpiperazin-1-yl)-1,3-benzodiazol-1-yl]methyl}pentyl]oxy}-4,5-difluorophenyl)-1-methyl-6-oxopyridine-3-carboxylic acid (INT-A111-5). A mixture of methyl 5-(2-{[(4R)-4-{[2-amino-6-(4-methylpiperazin-1-yl)-1,3-benzodiazol-1-yl]methyl}pentyl]oxy}-4,5-difluorophenyl)-1-methyl-6-oxopyridine-3-carboxylate (100 mg, 0.16 mmol, 1 equiv) and lithium hydroxide monohydrate (10 mg, 0.24 mmol, 1.5 equiv) in tetrahydrofuran (2 mL) and water (0.5 mL) was stirred for 2 h at room temperature under nitrogen atmosphere. The reaction was quenched with water (5 mL) and the mixture was extracted with dichloromethane (3×10 mL). The combined organic layers were washed with brine (3×5 mL), dried over anhydrous Na₂SO₄, and concentrated under reduced pressure. The residue was purified by Prep-TLC eluted with dichloromethane/methanol (10/1) to give 50 mg of 5-(2-{[(4R)-4-{[2-amino-6-(4-methylpiperazin-1-yl)-1,3-benzodiazol-1-yl]methyl}pentyl]oxy}-4,5-difluorophenyl)-1-methyl-6-oxopyridine-3-carboxylic acid (INT-A111-5) as a white solid (51%). LCMS: m/z (ESI), [M+H]⁺=595.20.

Step 6. Preparation of (12R)-4,5-difluoro-12,27-dimethyl-17-(4-methylpiperazin-1-yl)-8-oxa-14,21,23,27-tetraazapentacyclo[23.3.1.0\hat{ }{2,7}.0\hat{ }{14,22}.0\hat{ }{15,20}]nonacosa-1(29),2(7),3,5,15,17,19,21,25-nonaene-24,28-dione (EXAMPLE A111). A mixture of 5-(2-{[(4R)-4-{[2-amino-6-(4-methylpiperazin-1-yl)-1,3-benzodiazol-1-yl]methyl}pentyl]oxy}-4,5-difluorophenyl)-1-methyl-6-oxopyridine-3-carboxylic acid (40 mg, 0.06 mmol, 1 equiv), N,N,N,N-tetramethyl-O-(7-azabenzotriazol-1-yl)uronium hexafluorophospate (38 mg, 0.10 mmol, 1.5 equiv), and N,N-diisopropylethylamine (26 mg, 0.20 mmol, 3 equiv) in 1,4-dioxane (4 mL) was stirred for 3 h at room temperature under nitrogen atmosphere. The reaction was quenched with water (10 mL) and the mixture was extracted with dichloromethane (3×15 mL). The combined organic layers were washed with brine (3×10 mL), dried over anhydrous Na₂SO₄, and concentrated under reduced pressure. The residue was purified by Prep-TLC eluted with dichloromethane/methanol (5/1) to give 9 mg of (12R)-4,5-difluoro-12,27-dimethyl-17-(4-methylpiperazin-1-yl)-8-oxa-14,21,23,27-tetraazapentacyclo[23.3.1.0\hat{ }{2,7}.0\hat{ }{14,22}.0\hat{ }{15,20}]nonacosa-1(29),2(7),3,5,15,17,19,21,25-nonaene-24,28-dione (EXAMPLE A111) as a white solid (22%). LCMS: m/z (EST), [M+H]⁺=577.30. ¹H NMR (DMSO-d₆, 400 MHz) δ 0.92 (3H, d), 1.15-1.65 (3H, m), 2.00-2.10 (2H, m), 2.35-2.45 (4H, m), 2.50 (3H, s), 3.10-3.30 (4H, m), 3.32 (3H, s), 3.91-3.97 (2H, m), 4.05 (1H, d), 4.19 (1H, t), 6.88 (1H, dd), 7.11 (1H, s), 7.26 (1H, dd), 7.35 (1H, d), 7.76 (1H, dd), 8.38 (1H, d), 8.89 (1H, d), 12.31 (1H, s)

### Example A112

### 5,12,12,26-Tetramethyl-16-(4-methylpiperazin-1-yl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione

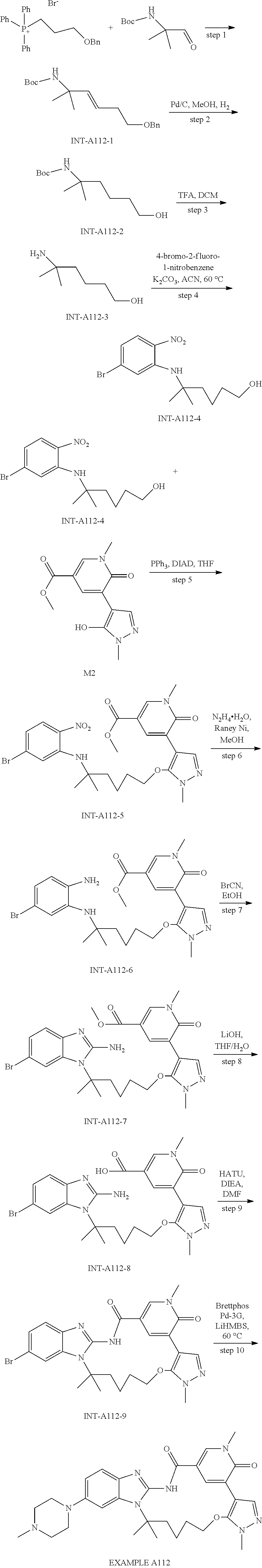

Step 1. Preparation of tert-butyl N-[(3E)-6-(benzyloxy)-2-methylhex-3-en-2-yl]carbamate (INT-A112-1). To a stirred mixture of (3-benzyloxypropyl)triphenylphosphonium bromide (14.5 g, 30 mmol, 1.1 equiv) in tetrahydrofuran (100 mL) was added KHMDS (5.9 g, 30 mmol, 1.1 equiv) dropwise at 0 °C under nitrogen atmosphere. The resulting mixture was stirred for 1 h at 0 °C under nitrogen atmosphere. To the above mixture was added tert-butyl N-(2-methyl-1-oxopropan-2-yl)carbamate (5 g, 26.7 mmol, 1 equiv) over 10 min at 0 °C. The resulting mixture was stirred overnight at room temperature. The reaction was quenched with water (50 mL) and the mixture was extracted with ethyl acetate (3×50 mL). The combined organic layers were washed with brine (3×50 mL), dried over anhydrous Na₂SO₄, and concentrated under reduced pressure. The residue was purified by Prep-TLC eluted with petroleum ether/ethyl acetate (5/1) to give 6 g of tert-butyl N-[(3E)-6-(benzyloxy)-2-methylhex-3-en-2-yl]carbamate (INT-A112-1) as a colorless oil (70%). ¹H NMR (CDCl₃, 400 MHz) δ 1.44 (9H, s), 1.46 (6H, s), 2.50-2.62 (2H, m), 3.45-3.52 (2H, m), 4.56 (2H, s), 5.32-5.42 (1H, m), 5.56-6.02 (1H, m), 7.29-7.39 (5H, m).

Step 2. Preparation of tert-butyl N-(6-hydroxy-2-methylhexan-2-yl)carbamate (INT-A 112-2). A mixture of tert-butyl N-[(3E)-6-(benzyloxy)-2-methylhex-3-en-2-yl]carbamate (2 g, 6.26 mmol, 1 equiv) and Pd/C (3.3 g, 31 mmol, 5 equiv) in methanol (40 mL) was stirred for 16 h at room temperature under hydrogen atmosphere. The mixture was filtered and the filter cake was washed with methanol (3×40 mL). The resulting solution was concentrated under reduced pressure to give 1.4 g of tert-butyl N-(6-hydroxy-2-methylhexan-2-yl)carbamate (INT-A112-2) as a colorless oil (97%). ¹H NMR (CDCl₃, 400 MHz) δ1.26 (6H, s), 1.32-1.41 (2H, m), 1.45 (9H, s), 1.60 (2H, d), 1.65-1.72 (2H, m), 3.62-3.73 (2H, m).

Step 3. Preparation of 5-amino-5-methylhexan-1-ol (INT-A112-3). A solution of tert-butyl N-(6-hydroxy-2-methylhexan-2-yl)carbamate (1.3 g, 5.6 mmol, 1 equiv) in trifluoroacetic acid (4 mL) and dichloromethane (16 mL) was was stirred for 2 h at room temperature under nitrogen atmosphere. The mixture was concentrated under reduced pressure. The residue was treated with saturated NaHCO₃ (aq.) (30 mL) and extracted with dichloromethane (3×30 mL). The combined organic layers were washed with brine (3×30 mL), dried over anhydrous Na₂SO₄, and concentrated under reduced pressure. The residue was purified by Prep-TLC eluted with dichloromethane/methanol (10/1) to give 680 mg of 5-amino-5-methylhexan-1-ol (INT-A112-3) as a colorless oil (92%). ¹H NMR (CD₃OD, 400 MHz) δ 1.35 (6H, d), 1.48-1.55 (2H, m), 1.65-1.72 (2H, m), 1.80-1.86 (2H, m), 4.40-4.49 (2H, m).

Step 4. Preparation of 5-((5-bromo-2-nitrophenyl)amino)-5-methylhexan-1-ol (INT-A112-4). A mixture of 5-amino-5-methylhexan-1-ol (680 mg, 5.18 mmol, 1 equiv), K₂CO₃ (2150 mg, 15.52 mmol, 3 equiv) and 4-bromo-2-fluoro-1-nitrobenzene (1140 mg, 5.18 mmol, 1 equiv) in acetonitrile was stirred for 16 h at 60 °C and cooled to room temperature. The resulting mixture was filtered and the filter cake was washed with dichloromethane (3×20 mL). The resulting solution was concentrated under reduced pressure. The residue was purified by Prep-TLC eluted with petroleum ether/ethyl acetate (1/1) to give 1.3 g of 5-((5-bromo-2-nitrophenyl)amino)-5-methylhexan-1-ol (INT-A112-4) as a yellow oil (76%). LCMS: m/z (ES+), [M+H]⁺=331.00.

Step 5. Preparation of methyl 5-(5-((5-((5-bromo-2-nitrophenyl)amino)-5-methylhexyl)oxy)-1-methyl-1H-pyrazol-4-yl)-1-methyl-6-oxo-1,6-dihydropyridine-3-carboxylate (INT-A112-5). To a stirred mixture of methyl 5-(5-hydroxy-1-methylpyrazol-4-yl)-1-methyl-6-oxopyridine-3-carboxylate (M2, 509 mg, 1.93 mmol, 1 equiv) and PPh₃ (1520 mg, 5.80 mmol, 3 equiv) in tetrahydrofuran (20 mL) was added 5-((5-bromo-2-nitrophenyl)amino)-5-methylhexan-1-ol (638 mg, 1.93 mmol, 1 equiv) and diisopropyl azodicarboxylate (1172 mg, 5.80 mmol, 3 equiv) at 0 °C under nitrogen atmosphere. The resulting mixture was stirred for 1 h at room temperature. The reaction was quenched with water (100 mL) and the mixture was extracted with ethyl acetate (3×100 mL). The combined organic layers were washed with brine (3×60 mL), dried over anhydrous Na₂SO₄, and concentrated under reduced pressure. The residue was purified by Prep-TLC eluted with dichloromethane/methanol (20/1) to give 610 mg methyl 5-[5-({5-[(5-bromo-2-nitrophenyl)amino]-5-methylhexyl}oxy)-1-methylpyrazol-4-yl]-1-methyl-6-oxopyridine-3-carboxylate (INT-A112-5) as a yellow solid (55%). LCMS: m/z (ESI), [M+H]⁺=578.30.

Step 6. Preparation of methyl 5-(5-((5-((2-amino-5-bromophenyl)amino)-5-methylhexyl)oxy)-1-methyl-1H-pyrazol-4-yl)-1-methyl-6-oxo-1,6-dihydropyridine-3-carboxylate (INT-A112-6). To a stirred mixture of methyl 5-[5-({5-[(5-bromo-2-nitrophenyl)amino]-5-methylhexyl}oxy)-1-methylpyrazol-4-yl]-1-methyl-6-oxopyridine-3-carboxylate (600 mg, 1.04 mmol, 1 equiv) and NH₂NH₂—H₂O (104 mg, 2.08 mmol, 2 equiv) in methanol (20 mL) was added Raney Ni (445.88 mg, 5.21 mmol, 5 equiv) at room temperature under nitrogen atmosphere. The resulting mixture was stirred for 2 h at room temperature. The resulting mixture was filtered and the filter cake was washed with methanol (3×20 mL). The resulting solution was concentrated under reduced pressure to give 450 mg of methyl 5-(5-((5-((2-amino-5-bromophenyl)amino)-5-methylhexyl)oxy)-1-methyl-1H-pyrazol-4-yl)-1-methyl-6-oxo-1,6-dihydropyridine-3-carboxylate (INT-A112-6) as a yellow solid. This material was used for the next reaction without further purification. LCMS: m/z (EST), [M+H]⁺=546.05.

Step 7. Preparation of methyl 5-(5-((5-(2-amino-6-bromo-1H-benzo[d]imidazol-1-yl)-5-methylhexyl)oxy)-1-methyl-1H-pyrazol-4-yl)-1-methyl-6-oxo-1,6-dihydropyridine-3-carboxylate (INT-A112-7). A mixture of methyl 5-[5-({5-[(2-amino-5-bromophenyl)amino]-5-methylhexyl}oxy)-1-methylpyrazol-4-yl]-1-methyl-6-oxopyridine-3-carboxylate (450 mg, 0.82 mmol, 1 equiv) and BrCN (174 mg, 1.65 mmol, 2 equiv) in ethanol (15 mL) was stirred for 2 h at room temperature. The reaction was quenched with water (20 mL) and the mixture was extracted with dichloromethane (3×20 mL). The combined organic layers were washed with brine (3×20 mL), dried over anhydrous Na₂SO₄, and concentrated under reduced pressure. The residue was purified by Prep-TLC eluted with dichloromethane/methanol (10/1) to give 300 mg of methyl 5-(5-{[5-(2-amino-6-bromo-1,3-benzodiazol-1-yl)-5-methylhexyl]oxy}-1-methylpyrazol-4-yl)-1-methyl-6-oxopyridine-3-carboxylate (INT-A112-7) as a yellow solid (64%).

LCMS: m/z (EST), [M+H]⁺=573.25. ¹H NMR (DMSO, 400 MHz) δ 1.24 (2H, s), 1.66-1.72 (2H, m), 1.77 (6H, s), 1.99-2.07 (2H, m), 3.58 (6H, s), 3.80 (3H, s), 3.85-3.90 (2H, m), 6.31 (2H, s), 7.10 (2H, d), 7.61 (1H, s), 7.97 (1H, s), 8.09 (1H, d), 8.45 (1H, d).

Step 8. Preparation of 5-(5-((5-(2-amino-6-bromo-1,3-benzo[d]imidazol-1-yl)-5-methylhexyl)oxy)-1-methyl-1H-pyrazol-4-yl)-1-methyl-6-oxo-1,6-dihydropyridine-3-carboxylic acid (INT-A112-8). To a stirred mixture of methyl 5-[5-({5-[(aminomethyl)(3-bromophenyl)amino]-5-methylhexyl}oxy)-1-methylpyrazol-4-yl]-1-methyl-6-oxopyridine-3-carboxylate (280 mg, 0.5 mmol, 1 equiv) in THE (6 mL) and H₂O (1.5 mL) was added LiOH·H₂O (63 mg, 1.5 mmol, 3 equiv) at room temperature. The resulting mixture was stirred for 16 h at room temperature under nitrogen atmosphere and concentrated under reduced pressure. The residue was purified by C18 flash column using water containing 10 mmol/L NH₄HCO₃ and 0.10% NH₃·H₂O and acetonitrile as mobile phase. Fractions containing the desired compound were evaporated to dryness to give 150 mg of 5-(5-{[5-(2-amino-6-bromo-1,3-benzodiazol-1-yl)-5-methylhexyl]oxy}-1-methylpyrazol-4-yl)-1-methyl-6-oxopyridine-3-carboxylic acid (INT-A112-8) as a white solid (54%). LCMS: m/z (ESI), [M+H]⁺=557.25.

Step 9. Preparation of 16-bromo-5,12,12,26-tetramethyl-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (INT-A112-9). A mixture of 5-(5-{[5-(2-amino-6-bromo-1,3-benzodiazol-1-yl)-5-methylhexyl]oxy}-1-methylpyrazol-4-yl)-1-methyl-6-oxopyridine-3-carboxylic acid (150 mg, 0.27 mmol, 1 equiv), N,N-diisopropylethylamine (104 mg, 0.81 mmol, 3 equiv), and N,N,N,N-tetramethyl-O-(7-azabenzotriazol-1-yl)uronium hexafluorophospate (205 mg, 0.54 mmol, 2 equiv) in dioxane (5 mL) was stirred for 2 h at 60 °C under nitrogen atmosphere. The mixture was cooled to room temperature. The reaction was quenched with water (20 mL) and the mixture was extracted with dichloromethane (3×20 mL). The combined organic layers were washed with brine (3×20 mL), dried over anhydrous Na₂SO₄, and concentrated under reduced pressure. The residue was purified by silica gel chromatography eluted with dichloromethane/methanol (10/1) to give 140 mg of 16-bromo-5,12,12,26-tetramethyl-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (INT-A112-9) as a white solid (96%). LCMS: m/z (EST), [M+H]⁺=539.20

Step 10. Preparation of 5,12,12,26-tetramethyl-16-(4-methylpiperazin-1-yl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (EXAMPLE A112). To a stirred mixture of 16-bromo-5,12,12,26-tetramethyl-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (70 mg, 0.13 mmol, 1 equiv), BrettPhos (7 mg, 0.013 mmol, 0.1 equiv), and 1-methyl-piperazine (65 mg, 0.65 mmol, 5 equiv) in dioxane (5 mL) was added LiHMDS (1.3 mL, 1.3 mmol, 10 equiv) dropwise at room temperature under nitrogen atmosphere. The mixture was stirred for 2 h at 60 °C under nitrogen atmosphere and cooled to room temperature. The reaction was quenched with sat. NH₄Cl (aq.) (10 mL) and the mixture was extracted with dichloromethane (3×50 mL). The combined organic layers were washed with brine (3×50 mL), dried over anhydrous Na₂SO₄, and concentrated under reduced pressure. The residue was purified by silica gel chromatography eluted with dichloromethane/methanol (12/1) to give 24.7 mg of 5,12,12,26-tetramethyl-16-(4-methylpiperazin-1-yl)-7-oxa-4,5,13,20,22,26-hexaazapentacyclo[22.3.1.0\hat{ }{2,6}.0\hat{ }{13,21}.0\hat{ }{14,19}]octacosa-1(28),2(6),3,14,16,18,20,24-octaene-23,27-dione (EXAMPLE 112) as an off-white solid (33%). LCMS: m/z (EST), [M+H]⁺=559.30. ¹H NMR (DMSO-d₆, 400 MHz) δ 1.20-1.26 (2H, m), 1.68-1.76 (4H, m), 1.92 (6H, s), 2.28 (3H, s), 2.40-2.60 (4H, m), 3.11 (4H, br s), 3.63 (3H, s), 3.70 (3H, s), 4.08 (2H, t), 6.92 (1H, dd), 7.27 (1H, d), 7.44 (1H, d), 8.27 (1H, d), 8.32 (1H, s), 8.81 (1H, d), 12.71 (1H, s).

### Example A113

### 6-Fluoro-15,21-dimethyl-5-(morpholin-4-yl)-23-oxa-2,9,11,15,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,17(34),18(22),19-octaene-12,16-dione

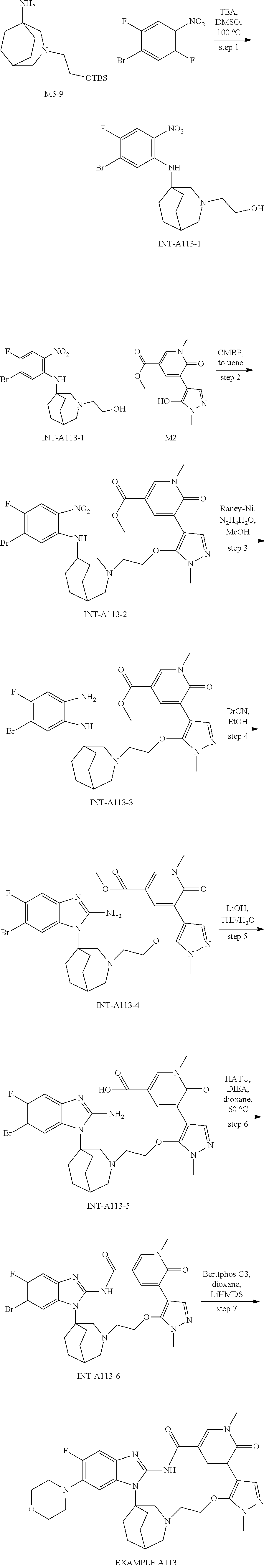

Step 1. Preparation of 2-{1-[(5-bromo-4-fluoro-2-nitrophenyl)amino]-3-azabicyclo[3.2.2]nonan-3-yl}ethanol (INT-A113-1). To a stirred mixture of 3-{2-[(tert-butyldimethylsilyl)oxy]ethyl}-3-azabicyclo[3.2.2]nonan-1-amine (M5-9, 700 mg, 2.35 mmol, 1 equiv) in DMSO (10 mL) were added trimethylamine (712 mg, 7.04 mmol, 3 equiv) and 1-bromo-2,5-difluoro-4-nitrobenzene (1.12 g, 4.69 mmol, 2 equiv) at room temperature. The resulting mixture was stirred for 2 h at 100 °C under nitrogen atmosphere and cooled to room temperature. The reaction was quenched with water (100 mL) and the mixture was extracted with ethyl acetate (3×100 mL). The combined organic layers were washed with brine (3×50 mL), dried over anhydrous Na₂SO₄, and concentrated under reduced pressure. The residue was purified by C18 FLASH using water and acetonitrile as mobile phase. Fractions containing the desired compound were evaporated to dryness to give 800 mg of 2-{1-[(5-bromo-4-fluoro-2-nitrophenyl)amino]-3-azabicyclo[3.2.2]nonan-3-yl}ethanol (INT-A113-1) as a yellow solid (85%). LCMS: m/z (ESI), [M+H]⁺=404.00. ¹H NMR (DMSO-d₆, 400 MHz) δ 1.49-1.61 (2H, m), 1.74-1.83 (4H, m), 1.90 (1H, s), 2.35-2.46 (2H, m), 2.55 (2H, t), 2.64 (2H, d), 2.86 (2H, s), 3.58 (2H, q), 4.43 (1H, t), 7.25 (1H, d), 7.99 (1H, s), 8.07 (1H, d).

Step 2. Preparation of methyl 5-[5-(2-{1-[(5-bromo-4-fluoro-2-nitrophenyl)amino]-3-azabicyclo[3.2.2]nonan-3-yl}ethoxy)-1-methylpyrazol-4-yl]-1-methyl-6-oxopyridine-3-carboxylate (INT-A113-2). To a stirred mixture of 2-{1-[(5-bromo-4-fluoro-2-nitrophenyl)amino]-3-azabicyclo[3.2.2]nonan-3-yl}ethanol (785 mg, 1.95 mmol, 1 equiv) and methyl 5-(5-hydroxy-1-methylpyrazol-4-yl)-1-methyl-6-oxopyridine-3-carboxylate (M2, 667 mg, 2.54 mmol, 1.3 equiv) in toluene (20 mL) was added 2-(tributyl-lambda5-phosphanylidene)acetonitrile (2826 mg, 11.71 mmol, 6 equiv) at room temperature under nitrogen atmosphere. The resulting mixture was stirred for 3 h at 100 °C. The mixture was cooled to room temperature. The reaction was quenched with water (100 mL) and the mixture was extracted with ethyl acetate (3×100 mL). The combined organic layers were washed with brine (3×100 mL), dried over anhydrous Na₂SO₄, and concentrated under reduced pressure. The residue was purified by Prep-TLC and eluted with petroleum ether/ethyl acetate (1/2) to give 670 mg of methyl 5-[5-(2-{1-[(5-bromo-4-fluoro-2-nitrophenyl)amino]-3-azabicyclo[3.2.2]nonan-3-yl}ethoxy)-1-methylpyrazol-4-yl]-1-methyl-6-oxopyridine-3-carboxylate (INT-A113-2) as a yellow solid (53%). LCMS: m/z (ESI), [M+H]⁺=647.15.

Step 3. Preparation of methyl 5-[5-(2-{1-[(2-amino-5-bromo-4-fluorophenyl)amino]-3-azabicyclo[3.2.2]nonan-3-yl}ethoxy)-1-methylpyrazol-4-yl]-1-methyl-6-oxopyridine-3-carboxylate (INT-A113-3). To a stirred mixture of methyl 5-[5-(2-{1-[(5-bromo-4-fluoro-2-nitrophenyl)amino]-3-azabicyclo[3.2.2]nonan-3-yl}ethoxy)-1-methylpyrazol-4-yl]-1-methyl-6-oxopyridine-3-carboxylate (660 mg, 1.02 mmol, 1 equiv), and Raney Ni (175 mg, 2.04 mmol, 2 equiv) in methanol (10 mL) was added NH₂NH₂·H₂O (153 mg, 3.06 mmol, 3 equiv) at room temperature. The resulting mixture was stirred for 1 h at room temperature. The reaction was quenched with water (80 mL) and the mixture was extracted with dichloromethane (3×80 mL). The combined organic layers were washed with brine (3×80 mL), dried over anhydrous Na₂SO₄, and concentrated under reduced pressure. The residue was purified by Prep-TLC and eluted with petroleum ether/ethyl acetate (1/32) to give 490 mg of methyl 5-[5-(2-{1-[(2-amino-5-bromo-4-fluorophenyl)amino]-3-azabicyclo[3.2.2]nonan-3-yl}ethoxy)-1-methylpyrazol-4-yl]-1-methyl-6-oxopyridine-3-carboxylate (INT-A113-3) as a brown solid (78%). LCMS: m/z (ESI), [M+H]⁺=617.10.

Step 4. Preparation of methyl 5-(5-{2-[1-(2-amino-6-bromo-5-fluoro-1,3-benzodiazol-1-yl)-3-azabicyclo[3.2.2]nonan-3-yl]ethoxy}-1-methylpyrazol-4-yl)-1-methyl-6-oxopyridine-3-carboxylate (INT-A113-4). To a stirred mixture of methyl 5-[5-(2-{1-[(2-amino-5-bromo-4-fluorophenyl)amino]-3-azabicyclo[3.2.2]nonan-3-yl}ethoxy)-1-methylpyrazol-4-yl]-1-methyl-6-oxopyridine-3-carboxylate (480 mg, 0.78 mmol, 1 equiv) in ethanol (5 mL) was added BrCN (165 mg, 1.55 mmol, 2 equiv) at room temperature. The resulting mixture was stirred for 1 h at 60 °C and cooled to room temperature. The reaction was quenched with sat. sodium hyposulfite (aq.) (60 mL) and the mixture was extracted with dichloromethane (3×50 mL). The combined organic layers were washed with brine (3×50 mL), dried over anhydrous Na₂SO₄, and concentrated under reduced pressure. The residue was purified by Prep-TLC and eluted with dichloromethane/methanol (20/1) to give 250 mg of methyl 5-(5-{2-[1-(2-amino-6-bromo-5-fluoro-1,3-benzodiazol-1-yl)-3-azabicyclo[3.2.2]nonan-3-yl]ethoxy}-1-methylpyrazol-4-yl)-1-methyl-6-oxopyridine-3-carboxylate (INT-A113-4) as a reddish solid (50%). LCMS: m/z (ESI), [M+H]⁺=642.15. ¹H NMR (DMSO-d₆, 400 MHz) δ 1.04-1.30 (2H, m), 1.81 (1H, s), 1.80-1.98 (5H, m), 2.69 (2H, s), 2.81-2.94 (4H, m), 3.56 (3H, s), 3.65-3.77 (6H, m), 4.12 (2H, t), 6.11 (2H, s), 7.05 (1H, d), 7.48 (1H, d), 7.97 (1H, s), 8.10 (1H, d), 8.38 (1H, d).

Step 5. Preparation of 5-(5-{2-[1-(2-amino-6-bromo-5-fluoro-1,3-benzodiazol-1-yl)-3-azabicyclo[3.2.2]nonan-3-yl]ethoxy}-1-methylpyrazol-4-yl)-1-methyl-6-oxopyridine-3-carboxylic acid (INT-A113-5). To a stirred solution of methyl 5-(5-{2-[1-(2-amino-6-bromo-5-fluoro-1,3-benzodiazol-1-yl)-3-azabicyclo[3.2.2]nonan-3-yl]ethoxy}-1-methylpyrazol-4-yl)-1-methyl-6-oxopyridine-3-carboxylate (220 mg, 0.34 mmol, 1 equiv) in tetrahydrofuran (4 mL) was added LiOH (21 mg, 0.86 mmol, 2.5 equiv) in H₂O (1 mL) at room temperature. The resulting mixture was stirred for 1 h at 60 °C and cooled to room temperature. The mixture was concentrated under reduced pressure. The residue was purified by C18 flash chromatography using water and acetonitrile as mobile phase. Fractions containing the desired compound were evaporated to dryness to give 190 mg of 5-(5-{2-[1-(2-amino-6-bromo-5-fluoro-1,3-benzodiazol-1-yl)-3-azabicyclo[3.2.2]nonan-3-yl]ethoxy}-1-methylpyrazol-4-yl)-1-methyl-6-oxopyridine-3-carboxylic acid (INT-A113-5) as a white solid (88%). LCMS: m/z (ESI), [M+H]⁺=630.10.

Step 6. Preparation of 5-bromo-6-fluoro-15,21-dimethyl-23-oxa-2,9,11,15,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,17(34),18(22),19-octaene-12,16-dione (INT-A113-6). To a stirred solution of 5-(5-{2-[1-(2-amino-6-bromo-5-fluoro-1,3-benzodiazol-1-yl)-3-azabicyclo[3.2.2]nonan-3-yl]ethoxy}-1-methylpyrazol-4-yl)-1-methyl-6-oxopyridine-3-carboxylic acid (190 mg, 0.30 mmol, 1 equiv) in 1,4-dioxane (5 mL) were added N,N-diisopropylethylamine (117 mg, 0.91 mmol, 3 equiv) and N,N,N,N-tetramethyl-O-(7-azabenzotriazol-1-yl)uronium hexafluorophospate (230 mg, 0.60 mmol, 2 equiv) at room temperature. The resulting mixture was stirred for 1 h at 60 °C under nitrogen atmosphere. The mixture was cooled to room temperature. The reaction was quenched with water (30 mL) and the mixture was extracted with dichloromethane (3×30 mL). The combined organic layers were washed with brine (3×30 mL), dried over anhydrous Na₂SO₄, and concentrated under reduced pressure. The residue was purified by Prep-TLC and eluted with dichloromethane/methanol (22/1) to give 150 mg of 5-bromo-6-fluoro-15,21-dimethyl-23-oxa-2,9,11,15,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,17(34),18(22),19-octaene-12,16-dione (INT-A113-6) as a light yellow solid (81%). LCMS: m/z (EST), [M+H]⁺=610.30.

Step 7. Preparation of 6-fluoro-15,21-dimethyl-5-(morpholin-4-yl)-23-oxa-2,9,11,15,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,17(34),18(22),19-octaene-12,16-dione (Example A113). To a stirred mixture of 5-bromo-6-fluoro-15,21-dimethyl-23-oxa-2,9,11,15,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,17(34),18(22),19-octaene-12,16-dione (50 mg, 0.08 mmol, 1 equiv), BrettPhos Pd G₃ (23 mg, 0.03 mmol, 0.3 equiv), and morpholine (22 mg, 0.25 mmol, 3 equiv) in 1,4-dioxane (4 mL) was added LiHMDS (0.82 mL, 0.82 mmol, 10 equiv) at room temperature under nitrogen atmosphere. The resulting mixture was stirred for 30 min at 60 °C under nitrogen atmosphere and cooled to room temperature. The reaction was quenched with sat. NH₄Cl (aq.) (80 mL) and the mixture was extracted with dichloromethane (3×80 mL). The combined organic layers were washed with brine (3×50 mL), dried over anhydrous Na₂SO₄, and concentrated under reduced pressure. The residue was purified by Prep-TLC and eluted with dichloromethane/methanol (25/1). The product was further purified by prep-HPLC with XBridge Shield RP18 OBD column using water containing 10 mmol/L NH₄HCO₃ and acetonitrile as mobile phase. Fractions containing the desired compound were evaporated to dryness to give 1.2 mg of 6-fluoro-15,21-dimethyl-5-(morpholin-4-yl)-23-oxa-2,9,11,15,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,17(34),18(22),19-octaene-12,16-dione (Example A113) as a white solid (2%). LCMS: m/z (EST), [M+H]⁺=617.35. ¹H NMR (CDCl₃, 400 MHz) δ 2.00-2.20 (9H, m), 2.84 (2H, d), 2.95 (2H, t), 3.06 (4H, t), 3.40-3.50 (2H, m), 3.71 (3H, s), 3.80 (3H, s), 3.91 (4H, t), 4.23 (2H, t), 7.01 (1H, d), 7.39 (1H, d), 8.23 (1H, d), 8.50 (1H, s), 9.19 (1H, d), 13.50 (1H, s).

TABLE 1

| Example | | [M + H]⁺ | |
|---|---|---|---|
| No. | Structure | (observed) | ¹H NMR δ (ppm) |
| A28 | 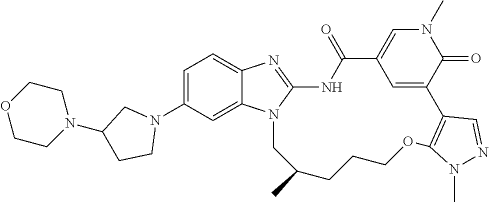 [SMILES: C[C@@H]1CCCOc2c(cnn2C)-c2cc(cn(C)c2=O)C(=O)Nc2nc3ccc(N4CCC(N5CCOCC5)C4)cc3n2C1] | 601.45 | ¹H NMR (DMSO-d₆, 400 MHz) δ 0.81 (3H, d), 1.40-1.49 (1H, m), 1.88-1.94 (3H, m), 2.20 (2H, br s), 2.80 (1H, br s), 2.89-2.99 (1H, m), 3.11 (1H, q), 3.18-3.56 (7H, m), 3.58-3.63 (7H, m), 3.72 (3H, s), 3.83-3.97 (2H, m), 4.11 (1H, d), 4.32-4.39 (1H, m), 6.47 (1H, d), 6.66 (1H, s), 7.33 (1H, d), 8.26 (1H, d), 8.35 (1H, s), 8.81 (1H, d), 12.23 (1H, s) |
| A29 | 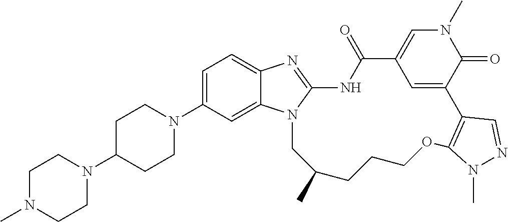 [SMILES: C[C@@H]1CCCOc2c(cnn2C)-c2cc(cn(C)c2=O)C(=O)Nc2nc3ccc(N4CCC(N5CCN(C)CC5)CC4)cc3n2C1] | 628.65 | ¹H NMR (DMSO-d₆, 400 MHz) δ 0.81 (3H, d), 1.41-1.46 (1H, m), 1.51-1.59 (2H, m), 1.89 (4H, t), 2.17 (6H, br s), 2.28-2.61 (4H, m), 2.65 (1H, t), 2.79 (1H, br s), 3.30 (4H, s), 3.62 (3H, s), 3.72 (3H, s), 3.67-3.77 (2H, m), 3.89-3.97 (2H, m), 4.09 (1H, d), 4.31-4.39 (1H, m), 6.86 (1H, d), 7.12 (1H, s), 7.35 (1H, d), 8.27 (1H, d), 8.35 (1H, s), 8.80 (1H, d), 12.35 (1H, s) |
| A30 | 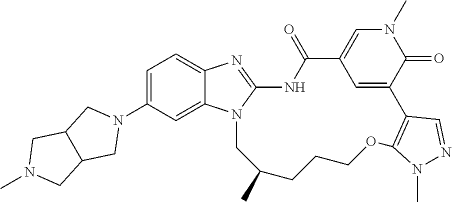 [SMILES: C[C@@H]1CCCOc2c(cnn2C)-c2cc(cn(C)c2=O)C(=O)Nc2nc3ccc(N4CC5CN(C)CC5C4)cc3n2C1] | 571.55 | ¹H NMR (DMSO-d₆, 400 MHz) δ 0.82 (3H, d), 1.41-1.47 (1H, m), 1.92 (2H, d), 2.10-2.19 (4H, m), 2.38-2.45 (2H, m), 2.54-2.61 (2H, m), 2.79 (1H, br s), 2.90 (2H, br s), 3.11 (2H, br s), 3.35-3.44 (2H, m), 3.62 (3H, s), 3.72 (3H, s), 3.79- 4.01 (2H, m), 4.10 (1H, d), 4.31- 4.40 (1H, m), 6.58 (1H, d), 6.77 (1H, s), 7.33 (1H, d), 8.26 (1H, s), 8.36 (1H, s), 8.81 (1H, s), 12.30 (1H, s) |
| A31 | 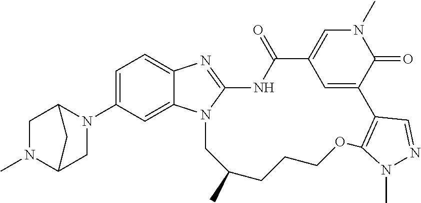 [SMILES: C[C@@H]1CCCOc2c(cnn2C)-c2cc(cn(C)c2=O)C(=O)Nc2nc3ccc(N4CC5CC4CN5C)cc3n2C1] | 557.45 | ¹H NMR (DMSO-d₆, 400 MHz) δ 0.81 (3H, d), 1.44 (1H, q), 1.79 (1H, d), 1.85-1.95 (3H, m), 2.19 (1H, s), 2.25 (3H, s), 2.50-2.54 (1H, m), 2.79 (2H, d), 3.18 (1H, d), 3.27-3.47 (2H, m), 3.62 (3H, s), 3.72 (3H, s), 3.83-3.96 (2H, m), 4.07-4.15 (1H, m), 4.35 (2H, s), 6.48 (1H, dd), 6.71 (1H, d), 7.31 (1H, d), 8.26 (1H, d), 8.35 (1H, s), 8.80 (1H, d), 12.25 (1H, s) |
| A32 | 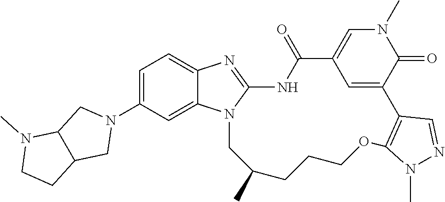 [SMILES: C[C@@H]1CCCOc2c(cnn2C)-c2cc(cn(C)c2=O)C(=O)Nc2nc3ccc(N4CC5CCN(C)C5C4)cc3n2C1] | 571.65 | ¹H NMR (DMSO-d₆, 400 MHz) δ 0.82 (3H, d), 1.41-1.50 (1H, m), 1.64 (1H, br s), 1.92 (2H, d), 2.06 (1H, br s), 2.15-2.40 (5H, m), 2.75- 3.07 (4H, m), 3.14-3.19 (2H, m), 3.27-3.45 (2H, m), 3.62 (3H, s), 3.72 (3H, s), 3.81-3.98 (2H, m), 4.11 (1H, d), 4.32-4.39 (1H, m), 6.46-6.57 (1H, m), 6.73 (1H, s), 7.33 (1H, d), 8.26 (1H, d), 8.36 (1H, s), 8.81 (1H, d), 12.29 (1H, s) |
| A33 | 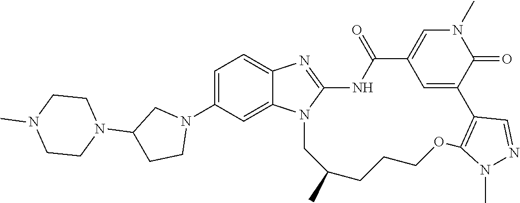 [SMILES: C[C@@H]1CCCOc2c(cnn2C)-c2cc(cn(C)c2=O)C(=O)Nc2nc3ccc(N4CCC(N5CCN(C)CC5)C4)cc3n2C1] | 614.45 | ¹H NMR (DMSO-d₆, 400 MHz) δ 0.81 (3H, d), 1.40-1.49 (1H, m), 1.74-2.01 (4H, m), 2.09-2.27 (6H, m), 2.26-2.49 (5H, m), 2.80 (1H, br s), 2.91 (1H, br s), 3.08 (1H, q), 3.21-3.43 (3H, m), 3.44-3.57 (1H, m), 3.62 (3H, s), 3.72 (3H, s), 3.83- 3.97 (2H, m), 4.10 (1H, d), 4.32- 4.40 (1H, m), 6.46 (1H, dd), 6.66 (1H, s), 7.33 (1H, d), 8.26 (1H, d), 8.35 (1H, s), 8.81 (1H, d), 12.27 (1H, s) |
| A34 | 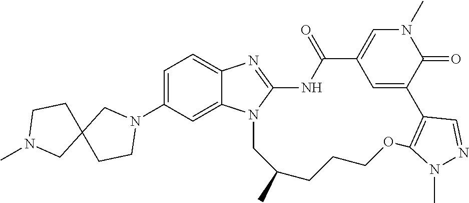 [SMILES: C[C@@H]1CCCOc2c(cnn2C)-c2cc(cn(C)c2=O)C(=O)Nc2nc3ccc(N4CCC5(CCN(C)C5)C4)cc3n2C1] | 585.55 | ¹H NMR (DMSO-d₆, 400 MHz) δ 0.82 (3H, d), 1.41-1.50 (1H, m), 1.75-1.86 (2H, m), 1.87-2.07 (4H, m), 2.19 (2H, br s), 2.26 (3H, s), 2.37-2.66 (4H, m), 2.80 (2H, br s), 3.15-3.35 (2H, m), 3.62 (3H, s), 3.72 (3H, s), 3.83-3.97 (2H, m), 4.09 (1H, d), 4.31-4.40 (1H, m), 6.43 (1H, dd), 6.62 (1H, s), 7.32 (1H, d), 8.26 (1H, d), 8.35 (1H, s), 8.81 (1H, d), 12.26 (1H, s) |
| A35 | 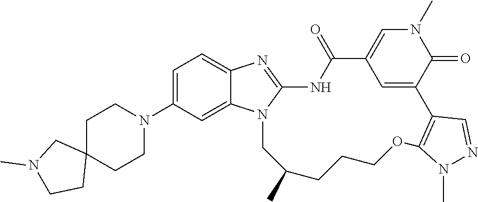 [SMILES: C[C@@H]1CCCOc2c(cnn2C)-c2cc(cn(C)c2=O)C(=O)Nc2nc3ccc(N4CCC5(CCN(C)C5)CC4)cc3n2C1] | 599.45 | ¹H NMR (DMSO-d₆, 400 MHz) δ 0.81 (3H, d), 1.40-1.49 (1H, m), 1.54-1.73 (6H, m), 1.91 (2H, d), 2.18 (1H, br s), 2.22 (3H, s), 2.33 (2H, s), 2.47-2.51 (2H, m), 2.79 (1H, s), 2.97-3.22 (4H, m), 3.62 (3H, s), 3.72 (3H, s), 3.88-3.99 (2H, m), 4.09 (1H, d), 4.31-4.40 (1H, m), 6.86 (1H, dd), 7.12 (1H, d), 7.34 (1H, d), 8.27 (1H, d), 8.35 (1H, s), 8.80 (1H, d), 12.30 (1H, s) |
| A36 | 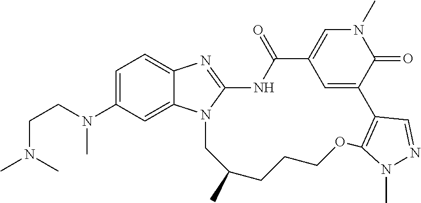 [SMILES: C[C@@H]1CCCOc2c(cnn2C)-c2cc(cn(C)c2=O)C(=O)Nc2nc3ccc(N(C)CCN(C)C)cc3n2C1] | 547.35 | ¹H NMR (DMSO-d₆, 400 MHz) δ 0.82 (3H, d), 1.44 (1H, q), 1.88- 1.95 (2H, m), 2.12-2.18 (1H, m), 2.19 (6H, s), 2.37-2.42 (2H, m), 2.76 (1H, d), 2.94 (3H, s), 3.37- 3.57 (2H, m), 3.62 (3H, s), 3.72 (3H, s), 3.82-3.98 (2H, m), 4.12 (1H, dd), 4.32-4.41 (1H, m), 6.63 (1H, dd), 6.80 (1H, d), 7.33 (1H, d), 8.26 (1H, d), 8.36 (1H, s), 8.80 (1H, d), 12.27 (1H, s) |
| A37 | 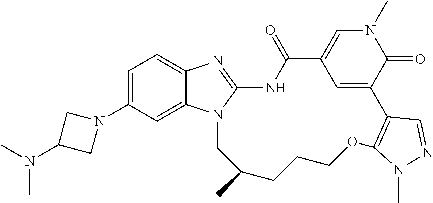 [SMILES: C[C@@H]1CCCOc2c(cnn2C)-c2cc(cn(C)c2=O)C(=O)Nc2nc3ccc(N4CC(N(C)C)C4)cc3n2C1] | 545.35 | ¹H NMR (DMSO-d₆, 400 MHz) δ 0.81 (3H, m), 1.43 (1H, q), 1.91 (2H, d), 2.17 (6H, s), 2.21-2.27 (1H, m), 2.73-2.85 (1H, m), 3.21- 3.31 (1H, m), 3.53-3.60 (2H, m), 3.62 (3H, s), 3.75 (3H, s), 3.79- 3.89 (1H, m), 3.90-4.00 (3H, m), 4.06-4.15 (1H, m), 4.31-4.41 (1H, m), 6.34 (1H, dd), 6.61 (1H, d), 7.34 (1H, d), 8.27 (1H, d), 8.35 (1H, s), 8.80 (1H, d), 12.33 (1H, s). |
| A38 | 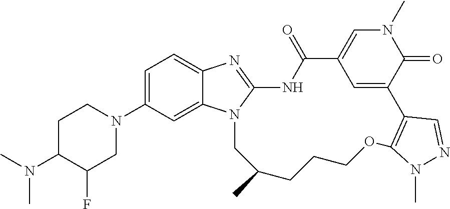 [SMILES: C[C@@H]1CCCOc2c(cnn2C)-c2cc(cn(C)c2=O)C(=O)Nc2nc3ccc(N4CCC(N(C)C)C(F)C4)cc3n2C1] | 591.35 | ¹H NMR (DMSO-d₆, 400 MHz) δ 0.82 (3H, d), 1.41-1.50 (1H, m), 1.90-1.94 (2H, m), 2.02-2.08 (1H, m), 2.15-2.21 (2H, m), 2.81-3.09 (9H, m), 3.45-3.60 (2H, m), 3.62 (3H, s), 3.72 (3H, s), 3.88-4.03 (2H, m), 4.07-4.13 (2H, m), 4.15 (1H, d), 5.31-5.51 (1H, m), 6.90 (1H, dt), 7.19 (1H, t), 7.38 (1H, d), 8.28 (1H, d), 8.36 (1H, s), 8.81 (1H, d), 12.39 (1H, s). |
| A39 | 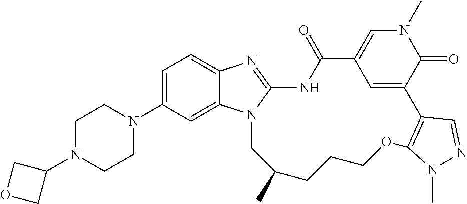 [SMILES: C[C@@H]1CCCOc2c(cnn2C)-c2cc(cn(C)c2=O)C(=O)Nc2nc3ccc(N4CCN(C5COC5)CC4)cc3n2C1] | 587.30 | ¹H NMR (DMSO-d₆, 400 MHz) δ 0.81 (3H, d), 1.35-1.51 (1H, m), 1.92 (2H, d), 2.13-2.27 (1H, m), 2.45 (4H, t), 2.72-2.86 (1H, m), 3.19 (4H, t), 3.42-3.57 (1H, m), 3.62 (3H, s), 3.72 (3H, s), 3.87- 3.99 (2H, m), 4.10 (1H, dd), 4.28- 4.41 (1H, m), 4.49 (2H, t), 4.59 (2H, t), 6.87 (1H, dd), 7.14 (1H, d), 7.37 (1H, d), 8.27 (1H, d), 8.35 (1H, s), 8.81 (1H, d), 12.35 (1H, s). |
| A40 | 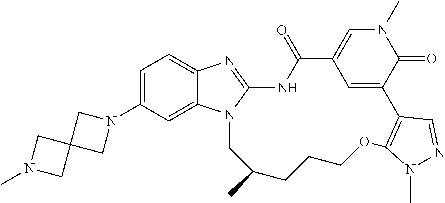 [SMILES: C[C@@H]1CCCOc2c(cnn2C)-c2cc(cn(C)c2=O)C(=O)Nc2nc3ccc(N4CC5(CN(C)C5)C4)cc3n2C1] | 557.30 | ¹H NMR (DMSO-d₆, 400 MHz) δ 0.80 (3H, d), 1.32-1.55 (1H, m), 1.92 (2H, d), 2.11-2.19 (1H, m), 2.20 (3H, s), 2.70-2.85 (1H, m), 3.27 (4H, s), 3.61 (3H, s), 3.72 (3H, s), 3.77-3.99 (2H, m), 3.93 (4H, s), 4.05-4.16 (1H, m), 4.30-4.40 (1H, m), 6.35 (1H, dd), 6.58 (1H, d), 7.32 (1H, d), 8.26 (1H, d), 8.35 (1H, s), 8.80 (1H, d), 12.24 (1H, s). |
| A41 | 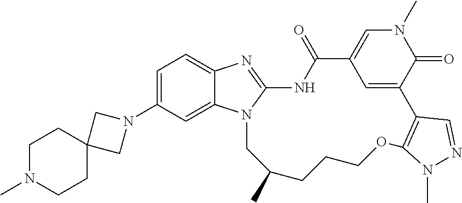 [SMILES: C[C@@H]1CCCOc2c(cnn2C)-c2cc(cn(C)c2=O)C(=O)Nc2nc3ccc(N4CC5(CCN(C)CC5)C4)cc3n2C1] | 585.25 | ¹H NMR (DMSO-d₆, 400 MHz) δ 0.81 (3H, d), 1.37-1.49 (1H, m), 1.83-2.02 (6H, m), 2.11-2.25 (1H, m), 2.26-2.43 (3H, m), 2.70-2.88 (1H, m), 3.59 (4H, br s), 3.62 (3H, s), 3.72 (3H, s), 3.77-3.87 (1H, m), 3.88-3.96 (1H, m), 4.10 (1H, d), 4.31-4.39 (1H, m), 6.32 (1H, dd), 6.56 (1H, d), 7.33 (1H, d), 8.26 (1H, d), 8.35 (1H, s), 8.80 (1H, d), 12.31 (1H, s). |
| A42 | 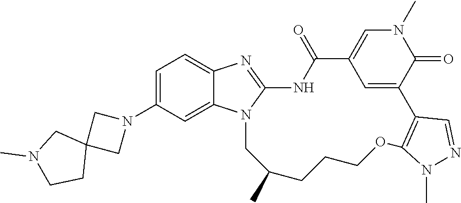 [SMILES: C[C@@H]1CCCOc2c(cnn2C)-c2cc(cn(C)c2=O)C(=O)Nc2nc3ccc(N4CC5(CCN(C)C5)C4)cc3n2C1] | 571.30 | ¹H NMR (DMSO-d₆, 400 MHz) δ 0.80 (3H, d), 1.32-1.49 (1H, m), 1.91 (2H, d), 2.05 (2H, t), 2.12- 2.22 (1H, m), 2.26 (3H, s), 2.38- 2.47 (2H, m), 2.68 (2H, s), 2.72- 2.85 (1H, m), 3.61 (3H, s), 3.65- 3.70 (2H, m), 3.72 (3H, s), 3.75- 3.88 (3H, m), 3.88-4.02 (1H, m), 4.10 (1H, dd), 4.30-4.40 (1H, m), 6.32 (1H, dd), 6.57 (1H, d), 7.33 (1H, d), 8.26(1H, s), 8.35 (1H, s), 8.80 (1H, d), 12.29 (1H, s). |
| A43 | 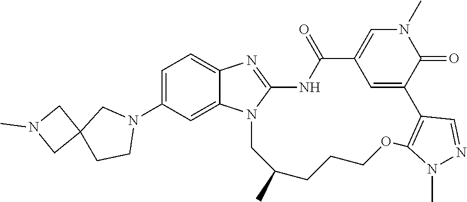 [SMILES: C[C@@H]1CCCOc2c(cnn2C)-c2cc(cn(C)c2=O)C(=O)Nc2nc3ccc(N4CCC5(CN(C)C5)C4)cc3n2C1] | 571.35 | ¹H NMR (DMSO-d₆, 400 MHz) δ 0.82 (3H, d), 1.42-1.46 (1H, m), 1.92 (2H, d), 2.10-2.20 (3H, m), 2.25 (3H, s), 2.77-2.83 (1H, m), 3.09-3.19 (4H, m), 3.21-3.45 (4H, m), 3.62 (3H, s), 3.72 (3H, s), 3.83- 3.96 (2H, m), 4.10 (1H, d), 4.29- 4.41 (1H, m), 6.45 (1H, dd), 6.64 (1H, d), 7.33 (1H, d), 8.26 (1H, d), 8.35 (1H, s), 8.80 (1H, d), 12.27 (1H, s). |
| A44 | 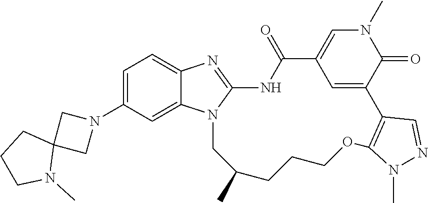 [SMILES: C[C@@H]1CCCOc2c(cnn2C)-c2cc(cn(C)c2=O)C(=O)Nc2nc3ccc(N4CC5(CCCN5C)C4)cc3n2C1] | 571.35 | ¹H NMR (DMSO-d₆, 400 MHz) δ 0.81 (3H, d), 1.37-1.49 (1H, m), 1.65-1.76 (2H, m), 1.89-1.93 (2H, m), 2.02-2.11 (2H, m), 2.16-2.22 (1H, m), 2.35 (3H, s), 2.65 (2H, t), 2.74-2.86 (1H, m), 3.57-3.62 (5H, m), 3.72 (3H, s), 3.78-3.96 (4H, m), 4.06-4.14 (1H, m), 4.35 (1H, t), 6.35 (1H, dd), 6.60 (1H, d), 7.33 (1H, d), 8.26 (1H, d), 8.35 (1H, s), 8.80 (1H, d), 12.31 (1H, s). |
| A45 | 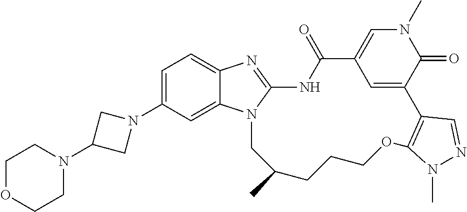 [SMILES: C[C@@H]1CCCOc2c(cnn2C)-c2cc(cn(C)c2=O)C(=O)Nc2nc3ccc(N4CC(N5CCOCC5)C4)cc3n2C1] | 587.70 | ¹H NMR (DMSO-d₆, 400 MHz) δ 0.81 (3H, d), 1.32-1.53 (1H, m), 1.91 (2H, d), 2.12-2.28 (1H, m), 2.35 (4H, s), 2.72-2.90 (1H, m), 3.19-3.31 (1H, m), 3.61 (8H, d), 3.72 (3H, s), 3.75-3.90 (1H, m), 3.94 (3H, d), 4.10 (1H, d), 4.30- 4.42 (1H, m), 6.33 (1H, dd), 6.60 (1H, s), 7.34 (1H, d), 8.27 (1H, s), 8.35 (1H, s), 8.80 (1H, d), 12.33 (1H, s). |
| A46 | 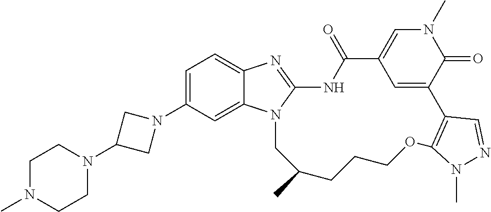 [SMILES: C[C@@H]1CCCOc2c(cnn2C)-c2cc(cn(C)c2=O)C(=O)Nc2nc3ccc(N4CC(N5CCN(C)CC5)C4)cc3n2C1] | 600.40 | ¹H NMR (DMSO-d₆, 400 MHz) δ 0.80 (3H, d), 1.43 (1H, q), 1.91 (2H, d), 2.16 (3H, s), 2.18-2.22 (1H, m), 2.33 (1H, br s), 2.73-2.87 (1H, m), 3.18-3.29 (1H, m), 3.49- 3.60 (2H, m), 3.62 (3H, s), 3.71 (3H, s), 3.78-3.88 (1H, m), 3.91- 3.99 (3H, m), 4.10 (1H, dd), 4.30- 4.40 (1H, m), 6.33 (1H, dd), 6.59 (1H, d), 7.33 (1H, d), 8.27 (1H, d), 8.35 (1H, s), 8.80 (1H, d), 12.33 (1H, s). |
| A47 | 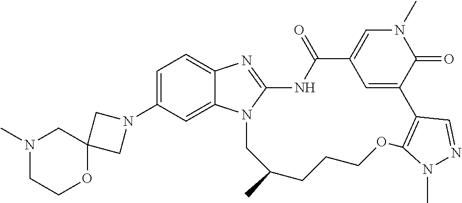 [SMILES: C[C@@H]1CCCOc2c(cnn2C)-c2cc(cn(C)c2=O)C(=O)Nc2nc3ccc(N4CC5(CN(C)CCO5)C4)cc3n2C1] | 587.35 | ¹H NMR (DMSO-d₆, 400 MHz) δ 0.81 (3H, d), 1.36-1.49 (1H, m), 1.91 (2H, d), 2.20-2.21 (1H, m), 2.23 (3H, s), 2.26-2.35 (2H, m), 2.36-2.59 (2H, m), 2.69-2.85 (1H, m), 3.55-3.68 (4H, m), 3.62 (3H, s), 3.72 (3H, s), 3.83 (3H, d), 3.89- 3.97 (1H, m), 4.10 (1H, d), 4.32- 4.38 (1H, m), 6.36 (1H, d), 6.64 (1H, d), 7.34 (1H, d), 8.27 (1H, d), 8.35 (1H, s), 8.80 (1H, d), 12.33 (1H, s). |
| A48 | 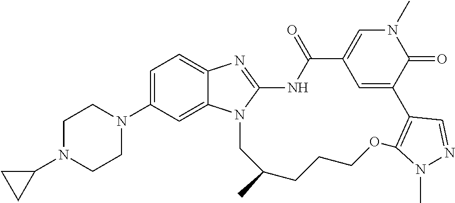 [SMILES: C[C@@H]1CCCOc2c(cnn2C)-c2cc(cn(C)c2=O)C(=O)Nc2nc3ccc(N4CCN(C5CC5)CC4)cc3n2C1] | 571.55 | ¹H NMR (DMSO-d₆, 400 MHz) δ 0.31-0.40 (2H, m), 0.42-0.55 (2H, m), 0.81 (3H, d), 1.35-1.52 (1H, m), 1.60-1.74 (1H, m), 1.85-1.97 (2H, m), 2.12-2.23 (1H, m), 2.71 (4H, t), 2.76-2.85 (1H, m), 3.11 (4H, t), 3.62 (3H, s), 3.72(3H, s), 3.88-3.98 (2H, m), 4.05-4.15 (1H, m), 4.28-4.43 (1H, m), 6.86 (1H, dd), 7.13 (1H, d), 7.35 (1H, d), 8.27 (1H, d), 8.35 (1H, s), 8.81 (1H, d), 12.34 (1H, s). |
| A49 | 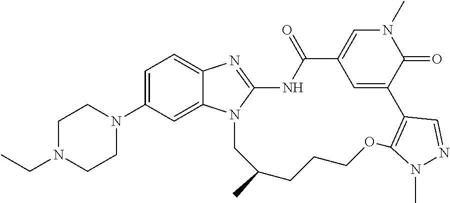 [SMILES: CCN1CCN(c2ccc3nc4n(c3c2)C[C@H](C)CCCOc2c(cnn2C)-c2cc(cn(C)c2=O)C(=O)N4)CC1] | 559.40 | ¹H NMR (DMSO-d₆, 400 MHz) δ 0.81 (3H, d), 1.05 (3H, t), 1.40- 1.49 (1H, m), 1.92 (2H, d), 2.13- 2.26 (1H, m), 2.39 (2H, q), 2.50- 2.60 (4H, m), 2.73-2.87 (1H, m), 3.16 (4H, t), 3.62 (3H, s), 3.72 (3H, s), 3.90-3.98 (2H, m), 4.08-4.12 (1H, m), 4.35 (1H, t), 6.86 (1H, dd), 7.12 (1H, d), 7.36 (1H, d), 8.27 (1H, d), 8.35 (1H, s), 8.81 (1H, d), 12.36 (1H, s). |
| A50 | 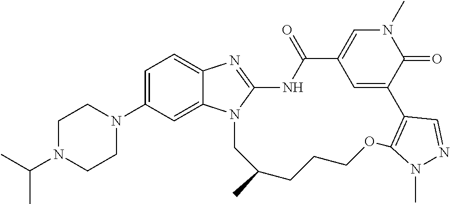 [SMILES: CC(C)N1CCN(c2ccc3nc4n(c3c2)C[C@H](C)CCCOc2c(cnn2C)-c2cc(cn(C)c2=O)C(=O)N4)CC1] | 573.40 | ¹H NMR (DMSO-d₆, 400 MHz) δ 0.81 (3H, d), 1.03 (6H, d), 1.44 (1H, q), 1.92 (2H, d), 2.10-2.26 (1H, m), 2.61 (4H, t), 2.65-2.74 (1H, m), 2.75-2.85 (1H, m), 3.14 (4H, t), 3.62 (3H, s), 3.72 (3H, s), 3.90-3.98 (2H, m), 4.06-4.14 (1H, m), 4.36 (1H, t), 6.86 (1H, dd), 7.11 (1H, d), 7.36 (1H, d), 8.27 (1H, d), 8.35 (1H, s), 8.81 (1H, d), 12.35 (1H, s). |
| A51 | 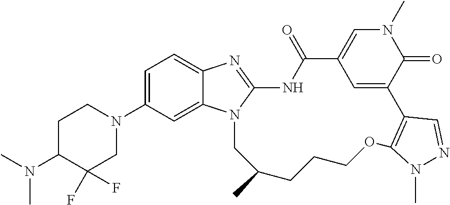 [SMILES: C[C@@H]1CCCOc2c(cnn2C)-c2cc(cn(C)c2=O)C(=O)Nc2nc3ccc(N4CCC(N(C)C)C(F)(F)C4)cc3n2C1] | 609.40 | ¹H NMR (DMSO-d₆, 400 MHz) δ 0.81 (3H, d), 1.22-1.28 (1H, m), 1.40-1.46 (1H, m), 1.91 (4H, t), 2.18-2.22 (1H, m), 2.40 (6H, s), 2.75-2.90 (4H, m), 3.62 (3H, s), 3.72 (3H, s), 3.75-4.03 (4H, m), 4.06-4.12 (1H, m), 4.36 (1H, s), 6.89 (1H, dd), 7.21 (1H, d), 7.36 (1H, d), 8.28 (1H, d), 8.36 (1H, d), 8.81 (1H, d), 12.39 (1H, s). |
| A52 | 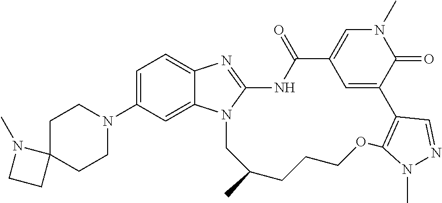 [SMILES: C[C@@H]1CCCOc2c(cnn2C)-c2cc(cn(C)c2=O)C(=O)Nc2nc3ccc(N4CCC5(CC4)CCN5C)cc3n2C1] | 585.55 | ¹H NMR (DMSO-d₆, 400 MHz) δ 0.80 (3H, d), 1.40-1.49 (1H, m), 1.69-1.83 (4H, m), 1.92 (4H, t), 2.14 (3H, s), 2.15-2.24 (1H, m), 2.65 (2H, t), 2.73-2.85 (1H, m), 3.12 (2H, s), 3.60-3.65 (1H, m), 3.62 (4H, t), 3.72 (3H, s), 3.93 (2H, t), 4.09 (1H, d), 4.35 (1H, t), 6.87 (1H, dd), 7.14 (1H, d), 7.34 (1H, d), 8.27 (1H, d), 8.35 (1H, s), 8.80 (1H, d), 12.34 (1H, s). |
| A53 | 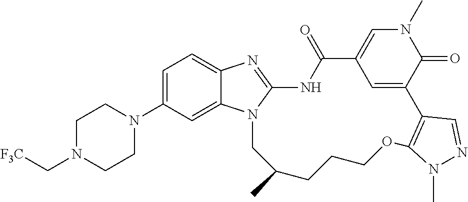 [SMILES: C[C@@H]1CCCOc2c(cnn2C)-c2cc(cn(C)c2=O)C(=O)Nc2nc3ccc(N4CCN(CC(F)(F)F)CC4)cc3n2C1] | 613.50 | ¹H NMR (DMSO-d₆, 400 MHz) δ 0.81 (3H, d), 1.20-1.32 (1H, m), 1.35-1.52 (1H, m), 1.81-1.99 (2H, m), 2.12-2.25 (1H, m), 2.81 (4H, s), 3.17 (4H, s), 3.25-3.55 (2H, m), 3.62 (3H, s), 3.72 (3H, s), 3.87- 3.97 (2H, m), 4.07-4.15 (1H, m), 4.28-4.42 (1H, m), 6.87 (1H, dd), 7.14 (1H, s), 7.37 (1H, d), 8.27 (1H, d), 8.36 (1H, s), 8.81 (1H, d). |
| A54 | 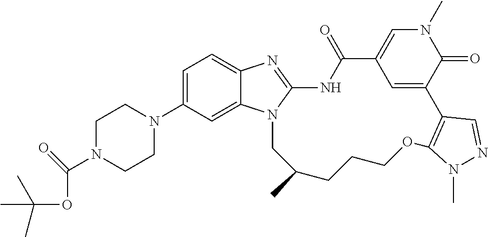 [SMILES: C[C@@H]1CCCOc2c(cnn2C)-c2cc(cn(C)c2=O)C(=O)Nc2nc3ccc(N4CCN(C(=O)OC(C)(C)C)CC4)cc3n2C1] | 631.25 | ¹H NMR (DMSO-d₆, 400 MHz) δ 0.81 (3H, d), 1.33-1.52 (1H, m), 1.44 (9H, s), 1.92 (2H, d), 2.13- 2.28 (1H, m), 2.72-2.85 (1H, m), 3.11 (4H, t), 3.50 (4H, t), 3.62 (3H, s), 3.72 (3H, s), 3.85-3.98 (2H, m), 4.11 (1H, d), 4.28-4.39 (1H, m), 6.89 (1H, dd), 7.18 (1H, d), 7.38 (1H, d), 8.27 (1H, d), 8.36 (1H, s), 8.81 (1H, d), 12.37 (1H, s). |
| A55 | 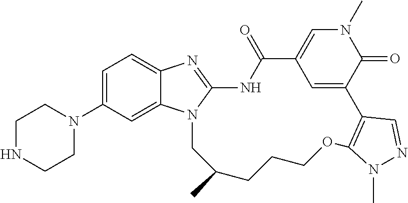 [SMILES: C[C@@H]1CCCOc2c(cnn2C)-c2cc(cn(C)c2=O)C(=O)Nc2nc3ccc(N4CCNCC4)cc3n2C1] | 531.45 | ¹H NMR (DMSO-d₆, 400 MHz) δ 0.81 (3H, d), 1.41-1.57 (1H, m), 1.87-2.12 (2H, m), 2.19-2.39 (1H, m), 2.78-2.92 (1H, m), 3.11-3.50 (8H, m), 3.52-3.71 (4H, m), 3.80 (3H, s), 3.86-3.98 (1H, m), 4.33 (1H, d), 4.40-4.55 (1H, m), 6.80- 7.04 (2H, m), 7.32 (1H, d), 8.01- 8.32 (1H, m), 8.33-8.61 (1H, m), 8.77-8.91 (1H, m). |
| A56 | 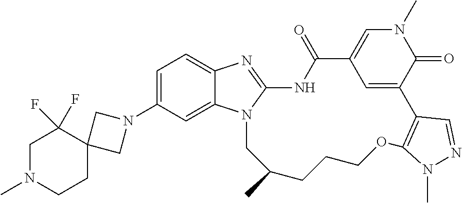 [SMILES: C[C@@H]1CCCOc2c(cnn2C)-c2cc(cn(C)c2=O)C(=O)Nc2nc3ccc(N4CC5(CCN(C)CC5(F)F)C4)cc3n2C1] | 621.40 | ¹H NMR (DMSO-d₆, 400 MHz) δ 0.82 (3H, d), 1.40-1.48 (1H, m), 1.92 (2H, d), 2.02-2.06 (2H, m), 2.13-2.22 (1H, m), 2.24 (3H, s), 2.35-2.65 (4H, m) 2.73-2.85 (1H, m), 3.62 (3H, s), 3.67 (2H, t), 3.72 (3H, s), 3.79-3.96 (4H, m), 4.11 (1H, d), 4.36 (1H, t), 6.35 (1H, dd), 6.65 (1H, s), 7.35 (1H, d), 8.27 (1H, d), 8.36 (1H, s), 8.81 (1H, d), 12.34 (1H, s). |
| A57 | 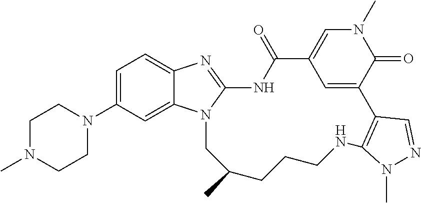 [SMILES: C[C@@H]1CCCNc2c(cnn2C)-c2cc(cn(C)c2=O)C(=O)Nc2nc3ccc(N4CCN(C)CC4)cc3n2C1] | 544.50 | 0.80 (3H, d), 1.35-1.43 (1H, m), 1.65-1.77 (1H, m), 1.85-1.97 (1H, m), 2.26 (3H, s), 2.48 (4H, t), 2.57- 2.65 (1H, m), 2.82-2.90 (1H, m), 3.16 (4H, t), 3.18-3.24 (1H, m), 3.61 (3H, s), 3.72 (3H, s), 3.81 (1H, t), 4.26 (1H, dd), 4.43 (1H, t), 6.86 (1H, dd), 7.10 (1H, d), 7.36 (1H, d), 8.19 (1H, s), 8.24 (1H, d), 8.69 (1H, d), 12.39 (1H, s). |
| A58 | 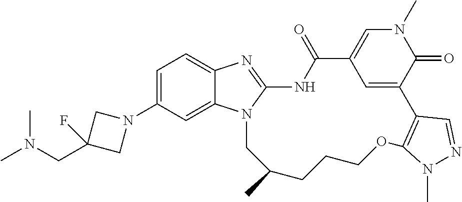 [SMILES: C[C@@H]1CCCOc2c(cnn2C)-c2cc(cn(C)c2=O)C(=O)Nc2nc3ccc(N4CC(F)(CN(C)C)C4)cc3n2C1] | 577.30 | ¹H NMR (DMSO-d₆, 400 MHz) δ 0.80 (3H, d), 1.10-1.21 (1H, m), 1.32-1.50 (1H, m), 1.91 (2H, d), 2.09-2.38 (1H, m), 2.28 (6H, s), 2.72-2.94 (2H, m), 3.62 (3H, s), 3.72 (3H, s), 3.80-4.14 (7H, m), 4.30-4.40 (1H, m), 6.39 (1H, d), 6.69 (1H, d), 7.36 (1H, dd), 8.27 (1H, d), 8.35 (1H, d), 8.80 (1H, d), 12.36 (1H, s). |
| A63A | 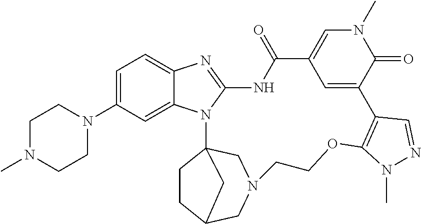 [SMILES: CN1CCN(c2ccc3nc4n(c3c2)C23CCC(CN(CCOc5c(cnn5C)-c5cc(cn(C)c5=O)C(=O)N4)C2)C3)CC1] | 598.30 | ¹H NMR (DMSO-d₆, 400 MHz) δ 1.53-1.77 (3H, m), 2.01 (1H, d), 2.25 (3H, s), 2.38-2.62 (7H, m), 2.60-2.76 (2H, m), 3.11 (4H, t), 3.17-3.46 (3H, m), 3.62 (3H, s), 3.63-3.69 (1H, m), 3.72 (3H, s), 4.55 (1H, t), 4.68 (1H, d), 6.90 (1H, dd), 7.20 (1H, s), 7.43 (1H, d), 8.27 (1H, d), 8.42 (1H, s), 8.82 (1H, d), 12.62 (1H, s). |
| | Isomer 1 | | |
| A63B | 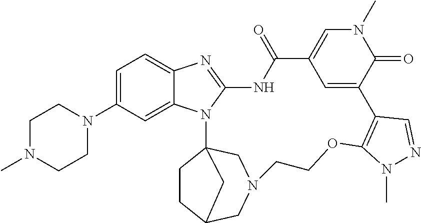 [SMILES: CN1CCN(c2ccc3nc4n(c3c2)C23CCC(CN(CCOc5c(cnn5C)-c5cc(cn(C)c5=O)C(=O)N4)C2)C3)CC1] | 598.30 | ¹H NMR (DMSO-d₆, 400 MHz) δ 1.53-1.77 (3H, m), 2.01 (1H, d), 2.25 (3H, s), 2.38-2.62 (7H, m), 2.60-2.76 (2H, m), 3.11 (4H, t), 3.17-3.46 (3H, m), 3.62 (3H, s), 3.63-3.69 (1H, m), 3.72 (3H, s), 4.55 (1H, t), 4.68 (1H, d), 6.90 (1H, dd), 7.20 (1H, s), 7.43 (1H, d), 8.27 (1H, d), 8.42 (1H, s), 8.82 (1H, d), 12.62 (1H, s). |
| | Isomer 2 | | |
| A64A | 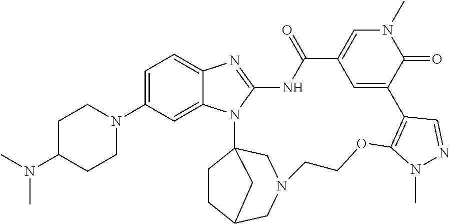 [SMILES: CN(C)C1CCN(c2ccc3nc4n(c3c2)C23CCC(CN(CCOc5c(cnn5C)-c5cc(cn(C)c5=O)C(=O)N4)C2)C3)CC1] | 626.25 | ¹H NMR (DMSO-d₆, 400 MHz) δ 1.42-1.80 (3H, m), 1.83-1.97 (2H, m), 2.01 (1H, d), 2.33 (6H, s), 2.42- 2.57 (4H, m), 2.61-2.82 (5H, m), 3.13-3.42 (6H, m), 3.62 (3H, s), 3.63-3.69 (1H, m), 3.72 (3H, s), 4.55 (1H, t), 4.67 (1H, d), 6.90 (1H, dd), 7.21 (1H, s), 7.41 (1H, d), 8.26 (1H, d), 8.42 (1H, s), 8.82 (1H, d), 12.59 (1H, s). |
| | Isomer 1 | | |
| A64B | 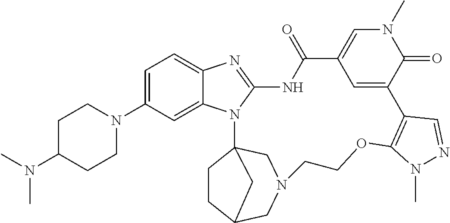 [SMILES: CN(C)C1CCN(c2ccc3nc4n(c3c2)C23CCC(CN(CCOc5c(cnn5C)-c5cc(cn(C)c5=O)C(=O)N4)C2)C3)CC1] | 626.25 | ¹H NMR (DMSO-d₆, 400 MHz) δ 1.42-1.80 (3H, m), 1.83-1.97 (2H, m), 2.01 (1H, d), 2.33 (6H, s), 2.42- 2.57 (4H, m), 2.61-2.82 (5H, m), 3.13-3.42 (6H, m), 3.62 (3H, s), 3.63-3.69 (1H, m), 3.72 (3H, s), 4.55 (1H, t), 4.67 (1H, d), 6.90 (1H, dd), 7.21 (1H, s), 7.41 (1H, d), 8.26 (1H, d), 8.42 (1H, s), 8.82 (1H, d), 12.59 (1H, s). |
| | Isomer 2 | | |
| A65A | 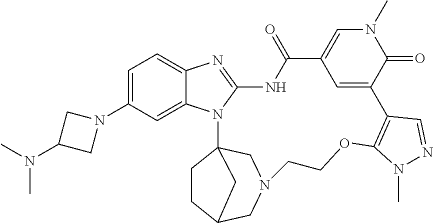 [SMILES: CN(C)C1CN(c2ccc3nc4n(c3c2)C23CCC(CN(CCOc5c(cnn5C)-c5cc(cn(C)c5=O)C(=O)N4)C2)C3)C1] | 598.35 | ¹H NMR (DMSO-d₆, 400 MHz) δ 1.55-1.75 (3H, m), 2.01 (1H, d), 2.14 (6H, s), 2.44-2.57 (4H, m), 2.71-2.76 (3H, m), 3.15-3.42 (4H, m), 3.51-3.59 (1H, m), 3.62 (3H, s), 3.63-3.69 (1H, m), 3.72 (3H, s), 3.94 (2H, q), 4.56 (1H, t), 4.69 (1H, d), 6.35 (1H, dd), 6.68 (1H, s), 7.39 (1H, d), 8.27 (1H, d), 8.43 (1H, s), 8.83 (1H, d), 12.55 (1H, s). |
| | Isomer 1 | | |
| A65B | 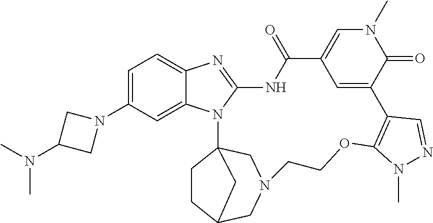 [SMILES: CN(C)C1CN(c2ccc3nc4n(c3c2)C23CCC(CN(CCOc5c(cnn5C)-c5cc(cn(C)c5=O)C(=O)N4)C2)C3)C1] | 598.35 | ¹H NMR (DMSO-d₆, 400 MHz) δ 1.55-1.75 (3H, m), 2.01 (1H, d), 2.14 (6H, s), 2.44-2.57 (4H, m), 2.71-2.76 (3H, m), 3.15-3.42 (4H, m), 3.51-3.59 (1H, m), 3.62 (3H, s), 3.63-3.69 (1H, m), 3.72 (3H, s), 3.94 (2H, q), 4.56 (1H, t), 4.69 (1H, d), 6.35 (1H, dd), 6.68 (1H, s), 7.39 (1H, d), 8.27 (1H, d), 8.43 (1H, s), 8.83 (1H, d), 12.55 (1H, s). |
| | Isomer 2 | | |
| A66A | 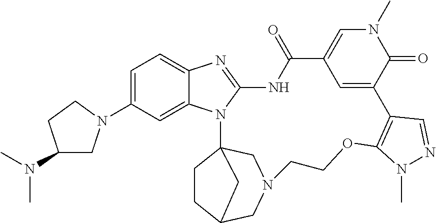 [SMILES: CN(C)[C@H]1CCN(c2ccc3nc4n(c3c2)C23CCC(CN(CCOc5c(cnn5C)-c5cc(cn(C)c5=O)C(=O)N4)C2)C3)C1] | 612.40 | ¹H NMR (DMSO-d₆, 400 MHz) δ 1.52-1.90 (4H, m), 2.00 (1H, d), 2.12-2.24 (1H, m), 2.26 (3H, s), 2.40-2.61 (4H, m), 2.68-2.99 (3H, m), 3.11 (1H, t), 3.17-3.49 (7H, m), 3.62 (3H, s), 3.63-3.69 (2H, m), 3.72 (3H, s), 4.56 (1H, t), 4.70 (1H, d), 6.50 (1H, dd), 6.77 (1H, d), 7.38 (1H, d), 8.26 (1H, d), 8.42 (1H, s), 8.82 (1H, d), 12.52 (1H, s). |
| | Isomer 1 | | |
| A66B | 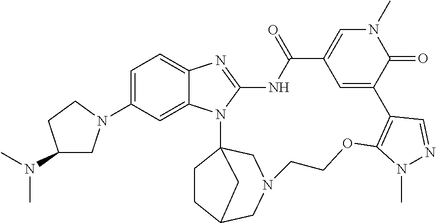 [SMILES: CN(C)[C@H]1CCN(c2ccc3nc4n(c3c2)C23CCC(CN(CCOc5c(cnn5C)-c5cc(cn(C)c5=O)C(=O)N4)C2)C3)C1] | 612.45 | ¹H NMR (DMSO-d₆, 400 MHz) δ 1.52-1.90 (4H, m), 2.00 (1H, d), 2.12-2.24 (1H, m), 2.26 (3H, s), 2.40-2.61 (4H, m), 2.68-2.99 (3H, m), 3.11 (1H, t), 3.17-3.49 (7H, m), 3.62 (3H, s), 3.63-3.69 (2H, m), 3.72 (3H, s), 4.56 (1H, t), 4.70 (1H, d), 6.50 (1H, dd), 6.77 (1H, d), 7.38 (1H, d), 8.26 (1H, d), 8.42 (1H, s), 8.82 (1H, d), 12.52 (1H, s). |
| | Isomer 2 | | |
| A66C | 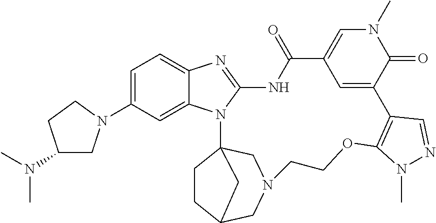 [SMILES: CN(C)[C@@H]1CCN(c2ccc3nc4n(c3c2)C23CCC(CN(CCOc5c(cnn5C)-c5cc(cn(C)c5=O)C(=O)N4)C2)C3)C1] | 612.45 | ¹H NMR (DMSO-d₆, 400 MHz) δ 1.52-1.90 (4H, m), 2.00 (1H, d), 2.12-2.21 (1H, m), 2.23 (3H, s), 2.41-2.59 (4H, m), 2.68-2.91 (3H, m), 3.07 (1H, t), 3.16-3.52 (7H, m), 3.62 (3H, s), 3.63-3.69 (2H, m), 3.72 (3H, s), 4.55 (1H, t), 4.68 (1H, d), 6.49 (1H, dd), 6.76 (1H, d), 7.38 (1H, d), 8.26 (1H, d), 8.42 (1H, s), 8.82 (1H, d), 12.51 (1H, s). |
| | Isomer 3 | | |
| A66D | 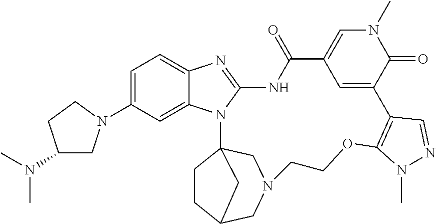 [SMILES: CN(C)[C@@H]1CCN(c2ccc3nc4n(c3c2)C23CCC(CN(CCOc5c(cnn5C)-c5cc(cn(C)c5=O)C(=O)N4)C2)C3)C1] | 612.40 | ¹H NMR (DMSO-d₆, 400 MHz) δ 1.52-1.90 (4H, m), 2.00 (1H, d), 2.12-2.21 (1H, m), 2.23 (3H, s), 2.41-2.59 (4H, m), 2.68-2.91 (3H, m), 3.07 (1H, t), 3.16-3.52 (7H, m), 3.62 (3H, s), 3.63-3.69 (2H, m), 3.72 (3H, s), 4.55 (1H, t), 4.68 (1H, d), 6.49 (1H, dd), 6.76 (1H, d), 7.38 (1H, d), 8.26 (1H, d), 8.42 (1H, s), 8.82 (1H, d), 12.51 (1H, s). |
| | Isomer 4 | | |
| A67A | 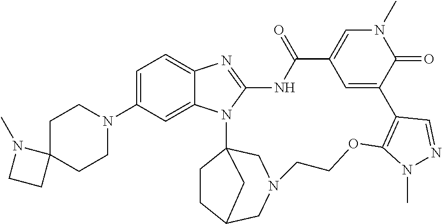 [SMILES: CN1CCC12CCN(c1ccc3nc4n(c3c1)C13CCC(CN(CCOc5c(cnn5C)-c5cc(cn(C)c5=O)C(=O)N4)C1)C3)CC2] | 638.45 | ¹H NMR (DMSO-d₆, 400 MHz) δ 1.55-1.84 (5H, m), 1.90 (2H, t), 2.01 (1H, d), 2.14 (3H, s), 2.37- 2.75 (6H, m), 3.11 (2H, s), 3.16- 3.57 (7H, m), 3.61 (3H, s), 3.62- 3.70 (2H, m), 3.72 (3H, s), 4.55 (1H, t), 4.67 (1H, d), 6.90 (1H, dd), 7.19 (1H, s), 7.40 (1H, d), 8.26 (1H, d), 8.42 (1H, s), 8.82 (1H, d), 12.58 (1H, s). |
| | Isomer 1 | | |
| A67B | 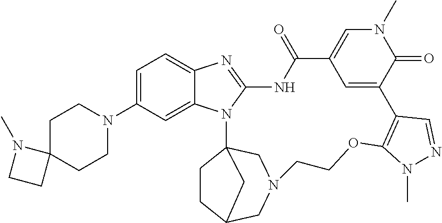 [SMILES: CN1CCC12CCN(c1ccc3nc4n(c3c1)C13CCC(CN(CCOc5c(cnn5C)-c5cc(cn(C)c5=O)C(=O)N4)C1)C3)CC2] | 638.45 | ¹H NMR (DMSO-d₆, 400 MHz) δ 1.55-1.84 (5H, m), 1.90 (2H, t), 2.01 (1H, d), 2.14 (3H, s), 2.37- 2.75 (6H, m), 3.11 (2H, s), 3.16- 3.57 (7H, m), 3.61 (3H, s), 3.62- 3.70 (2H, m), 3.72 (3H, s), 4.55 (1H, t), 4.67 (1H, d), 6.90 (1H, dd), 7.19 (1H, s), 7.40 (1H, d), 8.26 (1H, d), 8.42 (1H, s), 8.82 (1H, d), 12.58 (1H, s). |
| | Isomer 2 | | |
| A68A | 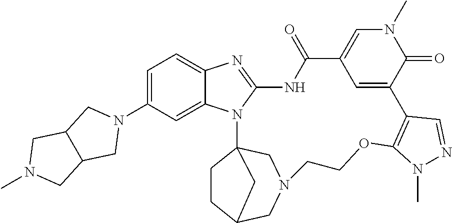 [SMILES: CN1CC2CN(c3ccc4nc5n(c4c3)C34CCC(CN(CCOc6c(cnn6C)-c6cc(cn(C)c6=O)C(=O)N5)C3)C4)CC2C1] | 624.45 | ¹H NMR (DMSO-d₆, 400 MHz) δ 1.53-1.77 (3H, m), 2.00 (1H, d), 2.22 (3H, s), 2.34-2.63 (8H, m), 2.64-2.73 (2H, m), 2.84-3.00 (2H, m), 3.04-3.15 (2H, m), 3.18-3.44 (4H, m), 3.62 (3H, s), 3.63-3.69 (1H, m), 3.72 (3H, s), 4.55 (1H, t), 4.68 (1H, d), 6.62 (1H, dd), 6.90 (1H, s), 7.38 (1H, d), 8.26 (1H, d), 8.42 (1H, s), 8.82 (1H, d), 12.55 (1H, s). |
| | Isomer 1 | | |
| A68B | 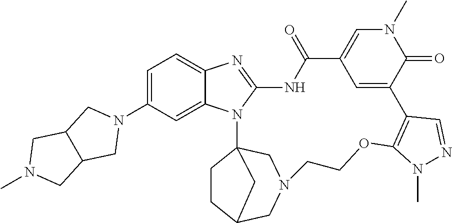 [SMILES: CN1CC2CN(c3ccc4nc5n(c4c3)C34CCC(CN(CCOc6c(cnn6C)-c6cc(cn(C)c6=O)C(=O)N5)C3)C4)CC2C1] | 624.45 | ¹H NMR (DMSO-d₆, 400 MHz) δ 1.53-1.77 (3H, m), 2.00 (1H, d), 2.22 (3H, s), 2.34-2.63 (8H, m), 2.64-2.73 (2H, m), 2.84-3.00 (2H, m), 3.04-3.15 (2H, m), 3.18-3.44 (4H, m), 3.62 (3H, s), 3.63-3.69 (1H, m), 3.72 (3H, s), 4.55 (1H, t), 4.68 (1H, d), 6.62 (1H, dd), 6.90 (1H, s), 7.38 (1H, d), 8.26 (1H, d), 8.42 (1H, s), 8.82 (1H, d), 12.55 (1H, s). |
| | Isomer 1 | | |
| A69A | 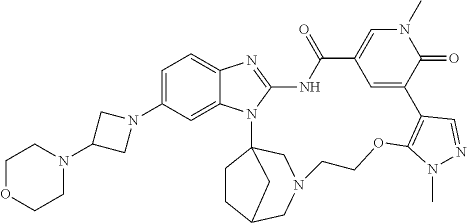 [SMILES: Cn1ncc2c1OCCN1CC3CCC(C3)(C1)n1c(nc3ccc(N4CC(N5CCOCC5)C4)cc31)NC(=O)c1cc-2c(=O)n(C)c1] | | ¹H NMR (DMSO-d₆, 400 MHz) δ 1.50-1.84 (3H, m), 1.96-2.02 (1H, m), 2.30-2.40 (4H, m), 2.42-2.60 (4H, m), 2.64-2.76 (3H, m), 3.20- 3.30 (3H, m), 3.55-3.69 (6H, m), 3.62 (3H, s), 3.71 (3H, s), 3.94 (2H, q), 4.55 (1H, t), 4.67 (1H, d), 6.34 (1H, dd), 6.66 (1H, d), 7.38 (1H, d), 8.22-8.28 (1H, d), 8.42 (1H, s), 8.81 (1H, d), 12.52 (1H, s). |
| | Isomer 1 | | |
| A69B | 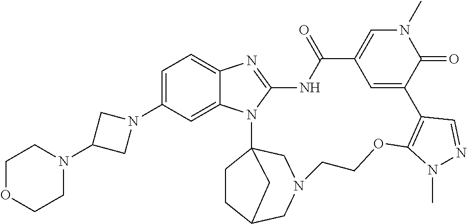 [SMILES: Cn1ncc2c1OCCN1CC3CCC(C3)(C1)n1c(nc3ccc(N4CC(N5CCOCC5)C4)cc31)NC(=O)c1cc-2c(=O)n(C)c1] | | ¹H NMR (DMSO-d₆, 400 MHz) δ 1.50-1.84 (3H, m), 1.96-2.02 (1H, m), 2.30-2.40 (4H, m), 2.42-2.60 (4H, m), 2.64-2.76 (3H, m), 3.20- 3.30 (3H, m), 3.55-3.69 (6H, m), 3.62 (3H, s), 3.71 (3H, s), 3.94 (2H, q), 4.55 (1H, t), 4.67 (1H, d), 6.34 (1H, dd), 6.66 (1H, d), 7.38 (1H, d), 8.22-8.28 (1H, d), 8.42 (1H, s), 8.81 (1H, d), 12.52 (1H, s). |
| | Isomer 2 | | |
| A70A | 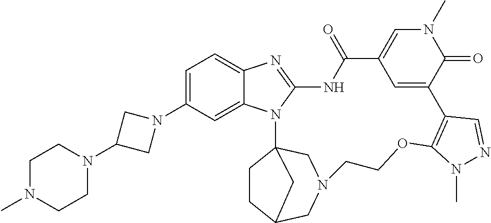 [SMILES: CN1CCN(C2CN(c3ccc4nc5n(c4c3)C34CCC(CN(CCOc6c(cnn6C)-c6cc(cn(C)c6=O)C(=O)N5)C3)C4)C2)CC1] | 653.35 | ¹H NMR (DMSO-d₆, 400 MHz) δ 1.50-1.80 (3H, m), 2.00 (1 H, d), 2.16 (3 H, s), 2.25-2.58 (8 H, m), 2.65-2.75 (3 H, m), 3.18-3.44 (6H, m), 3.57 (2H, q), 3.63 (3H, s), 3.63- 3.73 (1H, m), 3.73 (3H, s), 3.95 (2H, q), 4.56 (1H, t), 4.69 (1H, d), 6.35 (1H, dd), 6.67 (1H, s), 7.39 (1H, d), 8.27 (1H, d), 8.43 (1H, s), 8.83 (1H, d), 12.56 (1H, s). |
| | Isomer 1 | | |
| A70B | 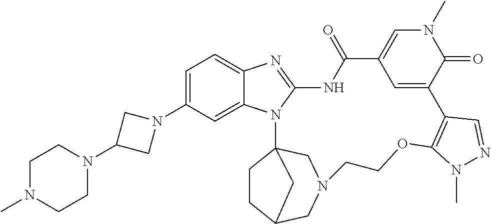 [SMILES: CN1CCN(C2CN(c3ccc4nc5n(c4c3)C34CCC(CN(CCOc6c(cnn6C)-c6cc(cn(C)c6=O)C(=O)N5)C3)C4)C2)CC1] | 653.35 | ¹H NMR (DMSO-d₆, 400 MHz) δ 1.50-1.80 (3H, m), 2.00 (1 H, d), 2.16 (3 H, s), 2.25-2.58 (8 H, m), 2.65-2.75 (3 H, m), 3.18-3.44 (6H, m), 3.57 (2H, q), 3.63 (3H, s), 3.63- 3.73 (1H, m), 3.73 (3H, s), 3.95 (2H, q), 4.56 (1H, t), 4.69 (1H, d), 6.35 (1H, dd), 6.67 (1H, s), 7.39 (1H, d), 8.27 (1H, d), 8.43 (1H, s), 8.83 (1H, d), 12.56 (1H, s). |
| | Isomer 2 | | |
| A71A | 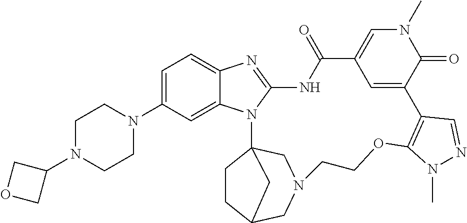 [SMILES: Cn1ncc2c1OCCN1CC3CCC(C3)(C1)n1c(nc3ccc(N4CCN(C5COC5)CC4)cc31)NC(=O)c1cc-2c(=O)n(C)c1] | 640.30 | ¹H NMR (DMSO-d₆, 400 MHz) 1.35-1.85 (3H, m), 2.02 (1H, d), 2.35-2.59 (3H, m), 2.44 (4H, t), 2.70-2.75 (3H, m), 3.15 (4H, t), 3.20-3.46 (3H, m), 3.63 (3H, s), 3.64-3.67 (1H, m), 3.72 (3H, s), 4.44-4.47 (2H, m), 4.54 (1H, s), 4.56-4.59 (2H, m), 4.68 (1H, d), 6.92 (1H, dd), 7.19 (1H, s), 7.43 (1H, d), 8.28 (1H, d), 8.42 (1H, s), 8.83 (1H, s), 12.60 (1H, s). |
| | Isomer 1 | | |
| A71B | 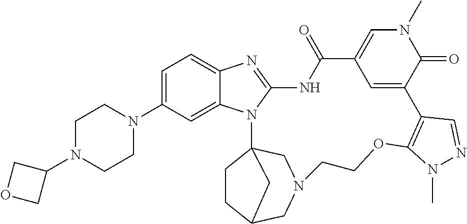 [SMILES: Cn1ncc2c1OCCN1CC3CCC(C3)(C1)n1c(nc3ccc(N4CCN(C5COC5)CC4)cc31)NC(=O)c1cc-2c(=O)n(C)c1] | 640.30 | ¹H NMR (DMSO-d₆, 400 MHz) 1.35-1.85 (3H, m), 2.02 (1H, d), 2.35-2.59 (3H, m), 2.44 (4H, t), 2.70-2.75 (3H, m), 3.15 (4H, t), 3.20-3.46 (3H, m), 3.63 (3H, s), 3.64-3.67 (1H, m), 3.72 (3H, s), 4.44-4.47 (2H, m), 4.54 (1H, s), 4.56-4.59 (2H, m), 4.68 (1H, d), 6.92 (1H, dd), 7.19 (1H, s), 7.43 (1H, d), 8.28 (1H, d), 8.42 (1H, s), 8.83 (1H, s), 12.60 (1H, s). |
| | Isomer 2 | | |
| A72A | 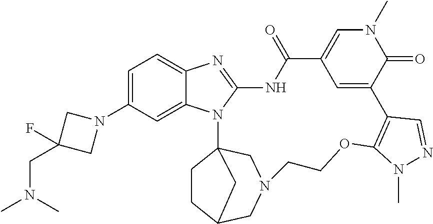 [SMILES: CN(C)CC1(F)CN(c2ccc3nc4n(c3c2)C23CCC(CN(CCOc5c(cnn5C)-c5cc(cn(C)c5=O)C(=O)N4)C2)C3)C1] | 630.35 | ¹H NMR (DMSO-d₆, 400 MHz) δ 1.45-1.83 (3H, m), 1.99 (1H, d), 2.25 (6H, s), 2.39-2.58 (4H, m), 2.62-2.76 (2H, m), 2.80 (1H, s), 3.08-3.42 (3H, m), 3.62 (3H, s), 3.63-3.69 (1H, m), 3.72 (3H, s),, 3.82-3.94 (2H, m), 3.95-4.05 (2H, m), 4.55(1H, t), 4.67 (1H, d), 6.41 (1H, t), 6.73 (1H, s), 7.41 (1H, d), 8.26 (1H, d), 8.42 (1H, d), 8.81 (1H, d), 12.59 (1H, s). |
| | Isomer 1 | | |
| A72B | 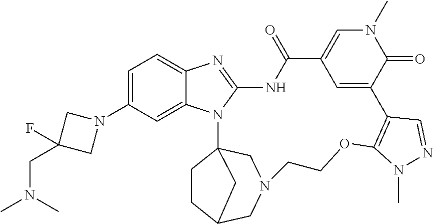 [SMILES: CN(C)CC1(F)CN(c2ccc3nc4n(c3c2)C23CCC(CN(CCOc5c(cnn5C)-c5cc(cn(C)c5=O)C(=O)N4)C2)C3)C1] | 630.40 | ¹H NMR (DMSO-d₆, 400 MHz) δ 1.45-1.83 (3H, m), 1.99 (1H, d), 2.25 (6H, s), 2.39-2.58 (4H, m), 2.62-2.76 (2H, m), 2.80 (1H, s), 3.08-3.42 (3H, m), 3.62 (3H, s), 3.63-3.69 (1H, m), 3.72 (3H, s), 3.82-3.94 (2H, m), 3.95-4.05 (2H, m), 4.55(1H, t), 4.67 (1H, d), 6.41 (1H, t), 6.73 (1H, s), 7.41 (1H, d), 8.26 (1H, d), 8.42 (1H, d), 8.81 (1H, d), 12.59 (1H, s). |
| | Isomer 2 | | |
| A73A | 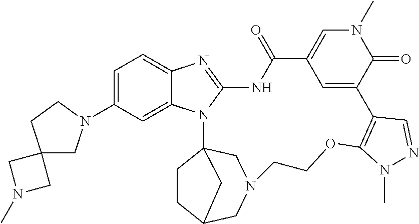 [SMILES: CN1CC2(CCN(c3ccc4nc5n(c4c3)C34CCC(CN(CCOc6c(cnn6C)-c6cc(cn(C)c6=O)C(=O)N5)C3)C4)C2)C1] | 624.45 | ¹H NMR (DMSO-d₆, 400 MHz) δ 1.51-1.78 (3H, m), 2.00 (1H, d), 2.13 (2H, t), 2.28 (3H, s), 2.38-2.58 (5H, m), 2.63-2.75 (2H, m), 3.19 (3H, s), 3.20-3.47 (6H, m), 3.62 (3H, s), 3.63-3.69 (1H, m), 3.72 (3H, s), 4.55 (1H, t), 4.68 (1H, d), 6.46 (1H, dd), 6.75 (1H, d), 7.37 (1H, d), 8.26 (1H, d), 8.42 (1H, s), 8.82 (1H, d), 12.51 (1H, s). |
| | Isomer 1 | | |
| A73B | 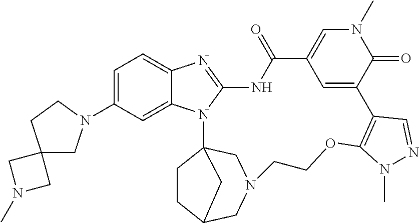 [SMILES: CN1CC2(CCN(c3ccc4nc5n(c4c3)C34CCC(CN(CCOc6c(cnn6C)-c6cc(cn(C)c6=O)C(=O)N5)C3)C4)C2)C1] | 624.40 | ¹H NMR (DMSO-d₆, 400 MHz) δ 1.51-1.78 (3H, m), 2.00 (1H, d), 2.13 (2H, t), 2.28 (3H, s), 2.38-2.58 (5H, m), 2.63-2.75 (2H, m), 3.19 (3H, s), 3.20-3.47 (6H, m), 3.62 (3H, s), 3.63-3.69 (1H, m), 3.72 (3H, s), 4.55 (1H, t), 4.68 (1H, d), 6.46 (1H, dd), 6.75 (1H, d), 7.37 (1H, d), 8.26 (1H, d), 8.42 (1H, s), 8.82 (1H, d), 12.51 (1H, s). |
| | Isomer 2 | | |
| A76A | 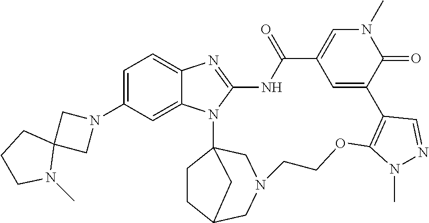 [SMILES: CN1CCCC12CN(c1ccc3nc4n(c3c1)C13CCC(CN(CCOc5c(cnn5C)-c5cc(cn(C)c5=O)C(=O)N4)C1)C3)C2] | 624.40 | ¹H NMR (DMSO-d₆, 400 MHz) δ 1.48-1.83 (4H, m), 1.93-2.16 (4H, m), 2.24-2.47 (5H, m), 2.59-2.80 (3H, m), 2.96 (3H, s), 3.09-3.27 (2H, m), 3.62 (3H, s), 3.63-3.69 (1H, m), 3.72 (3H, s), 3.81 (1H, d), 3.98 (1H, d), 4.18 (1H, t), 4.32 (1H, t), 4.56 (1H, t), 4.70 (1H, d), 6.43 (1H, dd), 6.75 (1H, s), 7.44 (1H, d), 8.28 (1H, d), 8.42 (1H, s), 8.82 (1H, d), 12.62 (1H, s). |
| | Isomer 1 | | |
| A76B | 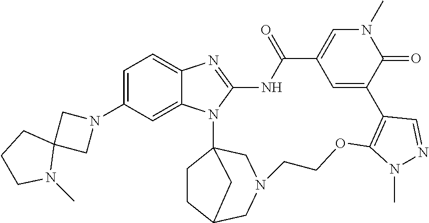 [SMILES: CN1CCCC12CN(c1ccc3nc4n(c3c1)C13CCC(CN(CCOc5c(cnn5C)-c5cc(cn(C)c5=O)C(=O)N4)C1)C3)C2] | 624.40 | ¹H NMR (DMSO-d₆, 400 MHz) δ 1.48-1.83 (4H, m), 1.93-2.16 (4H, m), 2.24-2.47 (5H, m), 2.59-2.80 (3H, m), 2.96 (3H, s), 3.09-3.27 (2H, m), 3.62 (3H, s), 3.63-3.69 (1H, m), 3.72 (3H, s), 3.81 (1H, d), 3.98 (1H, d), 4.18 (1H, t), 4.32 (1H, t), 4.55 (1H, t), 4.68 (1H, d), 6.43 (1H, dd), 6.68 (1H, s), 7.38 (1H, d), 8.26 (1H, d), 8.42 (1H, s), 8.81 (1H, d), 12.55 (1H, s). |
| | Isomer 2 | | |
| A77A | 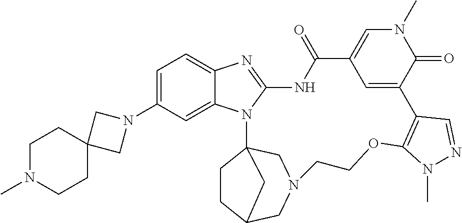 [SMILES: CN1CCC2(CC1)CN(c1ccc3nc4n(c3c1)C13CCC(CN(CCOc5c(cnn5C)-c5cc(cn(C)c5=O)C(=O)N4)C1)C3)C2] | 638.40 | ¹H NMR (DMSO-d₆, 400 MHz) δ 1.46-1.71 (3H, m), 1.74 (4H, t), 1.99 (1H, d), 2.15 (3H, s), 2.20- 2.60 (8H, m), 2.69-2.78 (3H, m), 3.12-3.24 (1H, m), 3.42-3.58 (4H, m), 3.62 (3H, s), 3.62-3.69 (1H, m), 3.72 (3H, s), 4.55 (1H, t), 4.67 (1H, d), 6.33 (1H, dd), 6.65 (1H, d), 7.37 (1H, d), 8.25 (1H, d), 8.41 (1H, s), 8.81 (1H, d), 12.52 (1H, s). |
| | Isomer 1 | | |
| A77B | 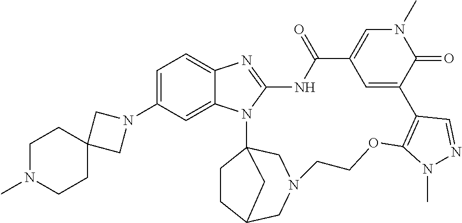 [SMILES: CN1CCC2(CC1)CN(c1ccc3nc4n(c3c1)C13CCC(CN(CCOc5c(cnn5C)-c5cc(cn(C)c5=O)C(=O)N4)C1)C3)C2] | 638.40 | ¹H NMR (DMSO-d₆, 400 MHz) δ 1.46-1.71 (3H, m), 1.74 (4H, t), 1.99 (1H, d), 2.15 (3H, s), 2.20- 2.60 (8H, m), 2.69-2.78 (3H, m), 3.12-3.24 (1H, m), 3.42-3.58 (4H, m), 3.62 (3H, s), 3.62-3.69 (1H, m), 3.72 (3H, s), 4.55 (1H, t), 4.67 (1H, d), 6.33 (1H, dd), 6.65 (1H, d), 7.37 (1H, d), 8.25 (1H, d), 8.41 (1H, s), 8.81 (1H, d), 12.52 (1H, s). |
| | Isomer 2 | | |
| A78A | 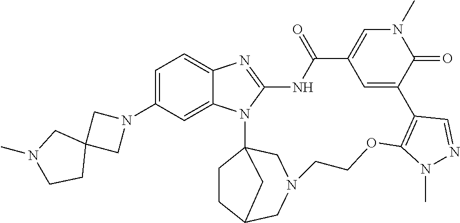 [SMILES: CN1CCC2(C1)CN(c1ccc3nc4n(c3c1)C13CCC(CN(CCOc5c(cnn5C)-c5cc(cn(C)c5=O)C(=O)N4)C1)C3)C2] | 624.35 | ¹H NMR (DMSO-d₆, 400 MHz) δ 1.44-1.78 (3H, m), 1.98 (1H, d), 2.04 (2H, t), 2.25 (3H, s), 2.30-2.47 (6H, m), 2.65 (3H, s), 2.67-2.79 (1H, m), 3.12-3.29 (1H, m), 3.58- 3.63 (3H, m), 3.62 (3H, s), 3.63- 3.69 (1H, m), 3.72 (3H, s), 3.74- 3.82 (2H, m), 4.55 (1H, t), 4.67 (1H, d), 6.33 (1H, d), 6.65 (1H, s), 7.38 (1H, d), 8.26 (1H, d), 8.41 (1H, s), 8.81 (1H, d), 12.55 (1H, s). |
| | Isomer 1 | | |
| A78B | 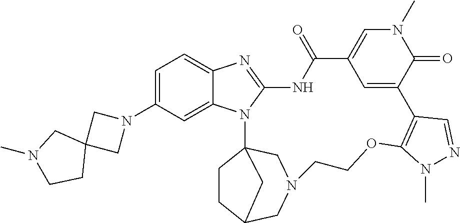 [SMILES: CN1CCC2(C1)CN(c1ccc3nc4n(c3c1)C13CCC(CN(CCOc5c(cnn5C)-c5cc(cn(C)c5=O)C(=O)N4)C1)C3)C2] | 624.35 | ¹H NMR (DMSO-d₆, 400 MHz) δ 1.44-1.78 (3H, m), 1.98 (1H, d), 2.04 (2H, t), 2.25 (3H, s), 2.30-2.47 (6H, m), 2.65 (3H, s), 2.67-2.79 (1H, m), 3.12-3.29 (1H, m), 3.58- 3.63 (3H, m), 3.62 (3H, s), 3.63- 3.69 (1H, m), 3.72 (3H, s), 3.74- 3.82 (2H, m), 4.55 (1H, t), 4.67 (1H, d), 6.33 (1H, d), 6.65 (1H, s), 7.38 (1H, d), 8.26 (1H, d), 8.41 (1H, s), 8.81 (1H, d), 12.55 (1H, s). |
| | Isomer 2 | | |
| A79A | 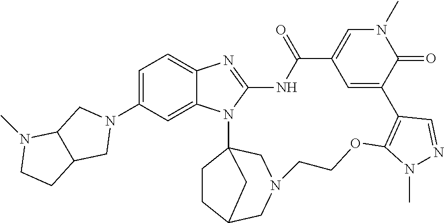 [SMILES: CN1CCC2CN(c3ccc4nc5n(c4c3)C34CCC(CN(CCOc6c(cnn6C)-c6cc(cn(C)c6=O)C(=O)N5)C3)C4)CC21] | 624.50 | ¹H NMR (CDCl₃, 400 MHz) δ 1.62-1.95 (6H, m), 2.05 (1H, t), 2.16-2.55 (5H, m), 2.54-2.89 (6H, m), 2.98-3.25 (2H, m), 3.27-3.42 (2H, m), 3.44-3.62 (4H, m), 3.70 (3H, s), 3.79 (3H, s), 4.71 (1H, t), 4.77 (1H, d), 6.62 (1H, d), 7.03 (1H, d), 7.14 (1H, d), 8.18 (1H, d), 8.62 (1H, s), 8.91 (1H, d), 12.29 (1H, s). |
| | Isomer 1 | | |
| A79B | 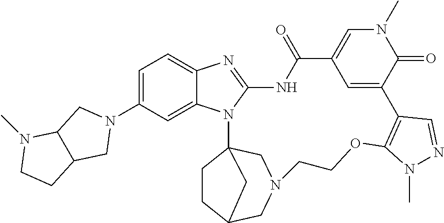 [SMILES: CN1CCC2CN(c3ccc4nc5n(c4c3)C34CCC(CN(CCOc6c(cnn6C)-c6cc(cn(C)c6=O)C(=O)N5)C3)C4)CC21] | 624.50 | ¹H NMR (CDCl₃, 400 MHz) δ 1.62-1.95 (6H, m), 2.05 (1H, t), 2.16-2.55 (5H, m), 2.54-2.89 (6H, m), 2.98-3.25 (2H, m), 3.27-3.42 (2H, m), 3.44-3.62 (4H, m), 3.70 (3H, s), 3.79 (3H, s), 4.71 (1H, t), 4.77 (1H, d), 6.64 (1H, d), 7.03 (1H, d), 7.15 (1H, d), 8.17 (1H, d), 8.62 (1H, s), 8.91 (1H, d), 12.30 (1H, s). |
| | Isomer 2 | | |
| A81A | 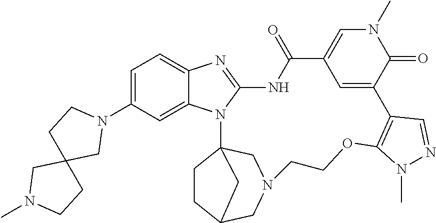 [SMILES: CN1CCC2(CCN(c3ccc4nc5n(c4c3)C34CCC(CN(CCOc6c(cnn6C)-c6cc(cn(C)c6=O)C(=O)N5)C3)C4)C2)C1] | 638.50 | ¹H NMR (CDCl₃, 400 MHz) δ 1.63-1.89 (6H, m), 2.01-2.20 (5H, m), 2.45-2.50 (3H, m), 2.53-2.70 (3H, m), 2.73-2.81 (2H, m), 2.87- 3.07 (2H, m), 3.11-3.23 (1H, m), 3.33 (1H, t), 3.41 (2H, d), 3.44- 3.62 (3H, m), 3.70 (3H, s), 3.78 (3H, s), 4.71 (1H, t), 4.78 (1H, d), 6.46 (1H, dd), 6.81 (1H, s), 7.14 (1H, d), 8.17 (1H, d), 8.62 (1H, s), 8.91 (1H, d), 12.27 (1H, s). |
| | Isomer 1 | | |
| A81B | 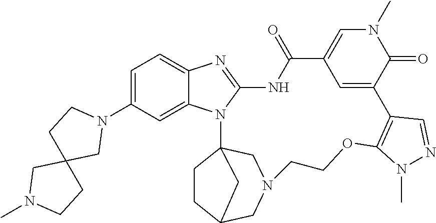 [SMILES: CN1CCC2(CCN(c3ccc4nc5n(c4c3)C34CCC(CN(CCOc6c(cnn6C)-c6cc(cn(C)c6=O)C(=O)N5)C3)C4)C2)C1] | 638.45 | ¹H NMR (CDCl₃, 400 MHz) δ 1.63-1.89 (6H, m), 2.01-2.20 (5H, m), 2.45-2.50 (3H, m), 2.53-2.70 (3H, m), 2.73-2.81 (2H, m), 2.87- 3.07 (2H, m), 3.11-3.23 (1H, m), 3.33 (1H, t), 3.41 (2H, d), 3.44- 3.62 (3H, m), 3.70 (3H, s), 3.78 (3H, s), 4.71 (1H, t), 4.78 (1H, d), 6.46 (1H, dd), 6.81 (1H, s), 7.14 (1H, d), 8.17 (1H, d), 8.62 (1H, s), 8.91 (1H, d), 12.27 (1H, s). |
| | Isomer 2 | | |
| A82A | 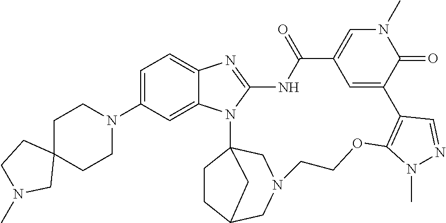 [SMILES: CN1CCC2(CCN(c3ccc4nc5n(c4c3)C34CCC(CN(CCOc6c(cnn6C)-c6cc(cn(C)c6=O)C(=O)N5)C3)C4)CC2)C1] | 652.60 | ¹H NMR (DMSO, 400 MHz) δ 1.53-1.77 (9H, m), 2.01 (1H, d), 2.22 (3H, s), 2.33 (2H, s), 2.40- 2.62 (6H, m), 2.64-2.76 (2H, m), 2.98-3.11 (4H, m), 3.20-3.26 (1H, m), 3.44 (1H, s), 3.62 (3H, s), 3.63- 3.69 (1H, m), 3.72 (3H, s), 4.55 (1H, t), 4.68 (1H, d), 6.90 (1H, dd), 7.18 (1H, d), 7.40 (1H, d), 8.26 (1H, d), 8.42 (1H, s), 8.82 (1H, d), 12.57 (1H, s). |
| | Isomer 1 | | |
| A82B | 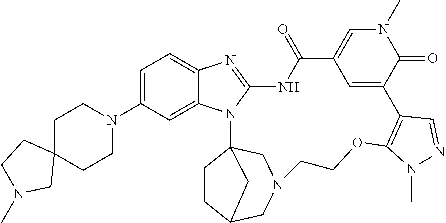 [SMILES: CN1CCC2(CCN(c3ccc4nc5n(c4c3)C34CCC(CN(CCOc6c(cnn6C)-c6cc(cn(C)c6=O)C(=O)N5)C3)C4)CC2)C1] | 652.35 | ¹H NMR (DMSO, 400 MHz) δ 1.53-1.77 (9H, m), 2.01 (1H, d), 2.22 (3H, s), 2.33 (2H, s), 2.40- 2.62 (6H, m), 2.64-2.76 (2H, m), 2.98-3.11 (4H, m), 3.20-3.26 (1H, m), 3.44 (1H, s), 3.62 (3H, s), 3.63- 3.69 (1H, m), 3.72 (3H, s), 4.55 (1H, t), 4.68 (1H, d), 6.90 (1H, dd), 7.18 (1H, d), 7.40 (1H, d), 8.26 (1H, d), 8.42 (1H, s), 8.82 (1H, d), 12.57 (1H, s). |
| | Isomer 2 | | |
| A83A | 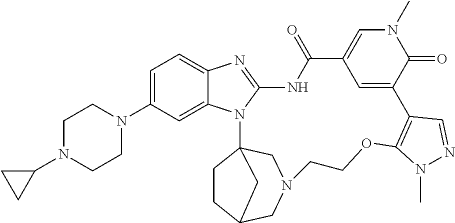 [SMILES: Cn1ncc2c1OCCN1CC3CCC(C3)(C1)n1c(nc3ccc(N4CCN(C5CC5)CC4)cc31)NC(=O)c1cc-2c(=O)n(C)c1] | 624.40 | ¹H NMR (DMSO-d₆, 400 MHz) δ 0.30-0.40 (2H, m), 0.40-0.50 (2H, m), 1.52-1.78 (4H, m), 2.01 (1H, d), 2.40-2.57 (4H, m), 2.68-2.72 (6H, m), 3.06 (4H, q), 3.30-3.34 (2H, m), 3.62 (3H, s), 3.63-3.69 (1H, m), 3.72 (3H, s), 4.55 (1H, t), 4.67 (1H, d), 6.89 (1H, dd), 7.17 (1H, d), 7.41 (1H, d), 8.27 (1H, d), 8.41 (1H, s), 8.82 (1H, d), 12.58 (1H, s). |
| | Isomer 1 | | |
| A83B | 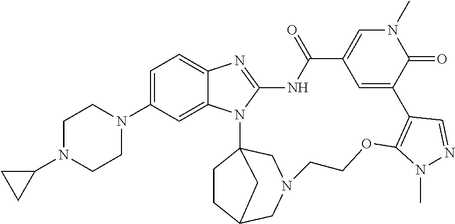 [SMILES: Cn1ncc2c1OCCN1CC3CCC(C3)(C1)n1c(nc3ccc(N4CCN(C5CC5)CC4)cc31)NC(=O)c1cc-2c(=O)n(C)c1] | 624.40 | ¹H NMR (DMSO-d₆, 400 MHz) δ 0.30-0.40 (2H, m), 0.40-0.50 (2H, m), 1.52-1.78 (4H, m), 2.01 (1H, d), 2.40-2.57 (4H, m), 2.68-2.72 (6H, m), 3.06 (4H, q), 3.30-3.34 (2H, m), 3.62 (3H, s), 3.63-3.69 (1H, m), 3.72 (3H, s), 4.55 (1H, t), 4.67 (1H, d), 6.89 (1H, dd), 7.17 (1H, d), 7.41 (1H, d), 8.27 (1H, d), 8.41 (1H, s), 8.82 (1H, d), 12.58 (1H, s). |
| | Isomer 2 | | |
| A85A | 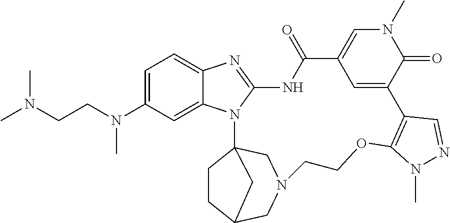 [SMILES: CN(C)CCN(C)c1ccc2nc3n(c2c1)C12CCC(CN(CCOc4c(cnn4C)-c4cc(cn(C)c4=O)C(=O)N3)C1)C2] | 600.30 | ¹H NMR (DMSO-d₆, 400 MHz) δ 1.56-1.71 (3H, m), 2.01-2.19 (1H, m), 2.19 (6H, s), 2.31-2.45 (5H, m), 2.51-2.59 (1H, m), 2.72-2.81 (2H, m), 2.92 (3H, s), 3.21-3.32 (1H, m), 3.35-3.48 (3H, m), 3.62 (3H, s), 3.63-3.69 (1H, m), 3.72 (3H, s), 4.55 (1H, t), 4.69 (1H, d), 6.66 (1H, d), 6.95 (1H, s), 7.38 (1H, d), 8.26 (1H, d), 8.41 (1H, s), 8.82 (1H, d), 12.58 (1H, s). |
| | Isomer 1 | | |
| A85B | 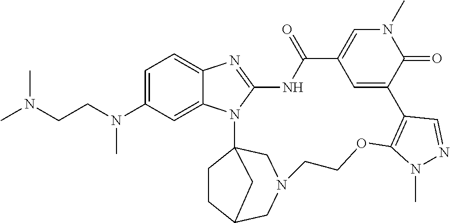 [SMILES: CN(C)CCN(C)c1ccc2nc3n(c2c1)C12CCC(CN(CCOc4c(cnn4C)-c4cc(cn(C)c4=O)C(=O)N3)C1)C2] | 600.30 | ¹H NMR (DMSO-d₆, 400 MHz) δ 1.56-1.71 (3H, m), 2.01-2.19 (1H, m), 2.19 (6H, s), 2.31-2.45 (5H, m), 2.51-2.59 (1H, m), 2.72-2.81 (2H, m), 2.92 (3H, s), 3.21-3.32 (1H, m), 3.35-3.48 (3H, m), 3.62 (3H, s), 3.63-3.69 (1H, m), 3.72 (3H, s), 4.55 (1H, t), 4.69 (1H, d), 6.66 (1H, d), 6.95 (1H, s), 7.38 (1H, d), 8.26 (1H, d), 8.41 (1H, s), 8.82 (1H, d), 12.58 (1H, s). |
| | Isomer 2 | | |
| A86A | 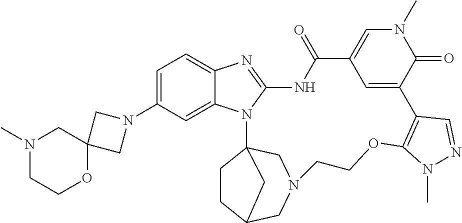 [SMILES: CN1CCOC2(C1)CN(c1ccc3nc4n(c3c1)C13CCC(CN(CCOc5c(cnn5C)-c5cc(cn(C)c5=O)C(=O)N4)C1)C3)C2] | 640.30 | ¹H NMR (DMSO-d₆, 400 MHz) δ 1.50-1.80 (3H, m), 1.99 (1H, d), 2.20-2.24 (3H, m), 2.28-2.32 (2H, m), 2.43-2.60 (4H, m), 2.65-2.75 (2H, m), 3.15-3.52 (4H, m), 3.58- 3.69 (8H, m), 3.72 (3H, s), 3.81 (2H, t), 4.55 (1H, t), 4.67 (1H, d), 6.37 (1H, dd), 6.69 (1H, d), 7.39 (1H, d), 8.26 (1H, d), 8.41 (1H, s), 8.81 (1H, d), 12.54 (1H, s). |
| | Isomer 1 | | |
| A86B | 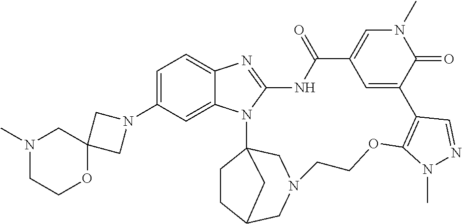 [SMILES: CN1CCOC2(C1)CN(c1ccc3nc4n(c3c1)C13CCC(CN(CCOc5c(cnn5C)-c5cc(cn(C)c5=O)C(=O)N4)C1)C3)C2] | 640.35 | ¹H NMR (DMSO-d₆, 400 MHz) δ 1.50-1.80 (3H, m), 1.99 (1H, d), 2.20-2.24 (3H, m), 2.28-2.32 (2H, m), 2.43-2.60 (4H, m), 2.65-2.75 (2H, m), 3.15-3.52 (4H, m), 3.58- 3.69 (8H, m), 3.72 (3H, s), 3.81 (2H, t), 4.55 (1H, t), 4.67 (1H, d), 6.37 (1H, dd), 6.70 (1H, d), 7.39 (1H, d), 8.26 (1H, d), 8.41 (1H, s), 8.82 (1H, d), 12.53 (1H, s). |
| | Isomer 2 | | |
| A87A | 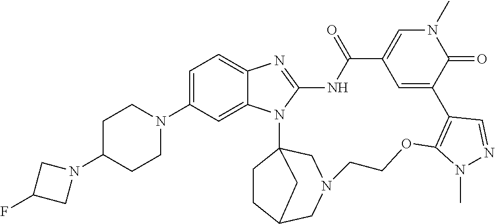 [SMILES: Cn1ncc2c1OCCN1CC3CCC(C3)(C1)n1c(nc3ccc(N4CCC(N5CC(F)C5)CC4)cc31)NC(=O)c1cc-2c(=O)n(C)c1] | 656.35 | ¹H NMR (DMSO-d₆, 400 MHz) δ 1.30-1.38 (2H, m), 1.51-1.83 (5H, m), 2.01 (1H, d), 2.15-2.26 (1H, m), 2.32-2.63 (4H, m), 2.66-2.81 (4H, m), 3.00-3.15 (2H, m), 3.15- 3.25 (1H, m), 3.52-3.7 (6H, m), 3.72 (3H, s), 4.55 (1H, t), 4.67 (1H, d), 5.04-5.27 (1H, m), 6.89 (1H, d), 7.18 (1H, s), 7.40 (1H, d), 8.26 (1H, d), 8.41 (1H, s), 8.82 (1H, d), 12.57 (1H, s). |
| | Isomer 1 | | |
| A87B | 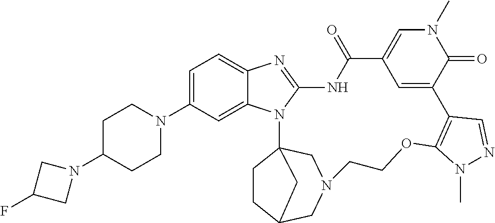 [SMILES: Cn1ncc2c1OCCN1CC3CCC(C3)(C1)n1c(nc3ccc(N4CCC(N5CC(F)C5)CC4)cc31)NC(=O)c1cc-2c(=O)n(C)c1] | 656.35 | ¹H NMR (DMSO-d₆, 400 MHz) δ 1.30-1.38 (2H, m), 1.51-1.83 (5H, m), 2.01 (1H, d), 2.15-2.26 (1H, m), 2.32-2.63 (4H, m), 2.66-2.81 (4H, m), 3.00-3.15 (2H, m), 3.15- 3.25 (1H, m), 3.52-3.7 (6H, m), 3.72 (3H, s), 4.55 (1H, t), 4.67 (1H, d), 5.04-5.27 (1H, m), 6.89 (1H, d), 7.18 (1H, s), 7.40 (1H, d), 8.26 (1H, d), 8.41 (1H, s), 8.82 (1H, d), 12.58 (1H, s). |
| | Isomer 1 | | |
| A88A | 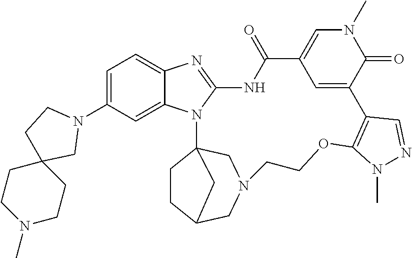 [SMILES: CN1CCC2(CC1)CCN(c1ccc3nc4n(c3c1)C13CCC(CN(CCOc5c(cnn5C)-c5cc(cn(C)c5=O)C(=O)N4)C1)C3)C2] | 652.2 | ¹H NMR (DMSO-d₆, 400 MHz) δ 1.49-1.77 (7H, m), 1.84 (2H, t), 1.98-2.06 (1H, m), 2.20 (3H, s), 2.32-2.40 (4H, m), 2.42-2.58 (5H, m), 2.70-2.74 (2H, m), 3.07-3.14 (2H, m),3.16-3.31 (2H, m), 3.38- 3.41 (1H, m), 3.62 (3H, s), 3.63- 3.69 (1H, m), 3.72 (3H, s), 4.55 (1H, t), 4.69 (1H, d), 6.47 (1H, d), 6.74 (1H, s), 7.37 (1H, d), 8.28 (1H, d), 8.42 (1H, s), 8.82 (1H, s), 12.50 (1H, s). |
| | Isomer 1 | | |
| A88B | 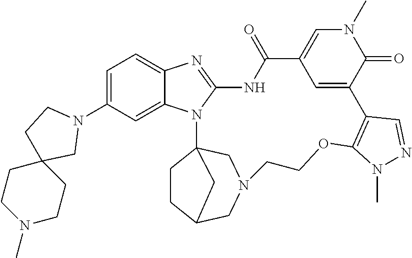 [SMILES: CN1CCC2(CC1)CCN(c1ccc3nc4n(c3c1)C13CCC(CN(CCOc5c(cnn5C)-c5cc(cn(C)c5=O)C(=O)N4)C1)C3)C2] | 652.45 | ¹H NMR (DMSO-d₆, 400 MHz) δ 1.49-1.77 (7H, m), 1.84 (2H, t), 1.98-2.06 (1H, m), 2.20 (3H, s), 2.32-2.40 (4H, m), 2.42-2.58 (5H, m), 2.70-2.74 (2H, m), 3.07-3.14 (2H, m),3.16-3.31 (2H, m), 3.38- 3.41 (1H, m), 3.62 (3H, s), 3.63- 3.69 (1H, m), 3.72 (3H, s), 4.55 (1H, t), 4.69 (1H, d), 6.46 (1H, d), 6.74 (1H, s), 7.37 (1H, d), 8.28 (1H, d), 8.42 (1H, s), 8.82 (1H, s), 12.50 (1H, s). |
| | Isomer 2 | | |
| A89A | 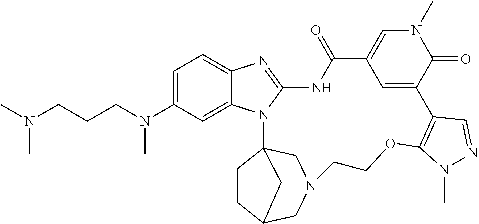 [SMILES: CN(C)CCCN(C)c1ccc2nc3n(c2c1)C12CCC(CN(CCOc4c(cnn4C)-c4cc(cn(C)c4=O)C(=O)N3)C1)C2] | 614.35 | ¹H NMR (DMSO-d₆, 400 MHz) δ 1.51-1.72 (5H, m), 1.98-2.04 (1H, m), 2.14 (6H, s), 2.20-2.26 (2H, m), 2.40-2.46 (2H, m), 2.53-2.57 (2H, m), 2.70-2.76 (2H, m), 2.88 (3H, s), 3.20-3.26 (2H, m), 3.32- 3.38 (2H, m), 3.62 (3H, s), 3.64- 3.68 (1H, m), 3.72 (3H, s), 4.55 (1H, t), 4.69 (1H, d), 6.67 (1H, dd), 6.99 (1H, d), 7.37 (1H, d), 8.26 (1H, d), 8.41 (1H, s), 8.82 (1H, d), 12.50 (1H, s). |
| | Isomer 1 | | |
| A89B | 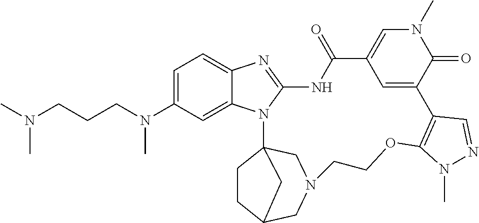 [SMILES: CN(C)CCCN(C)c1ccc2nc3n(c2c1)C12CCC(CN(CCOc4c(cnn4C)-c4cc(cn(C)c4=O)C(=O)N3)C1)C2] | 614.35 | ¹H NMR (DMSO-d₆, 400 MHz) δ 1.52-1.81 (5H, m), 1.98-2.04 (1H, m), 2.16 (6H, s), 2.22-2.28 (2H, m), 2.40-2.46 (3H, m), 2.53-2.57 (1H, m), 2.70-2.76 (2H, m), 2.88 (3H, s), 3.20-3.24 (2H, m), 3.32- 3.36 (2H, m), 3.62 (3H, s), 3.64- 3.68 (1H, m), 3.72 (3H, s), 4.55 (1H, t), 4.69 (1H, d), 6.68 (1H, dd), 6.99 (1H, d), 7.37 (1H, d), 8.26 (1H, d), 8.41 (1H, s), 8.82 (1H, d), 12.51 (1H, s). |
| | Isomer 2 | | |
| A90A | 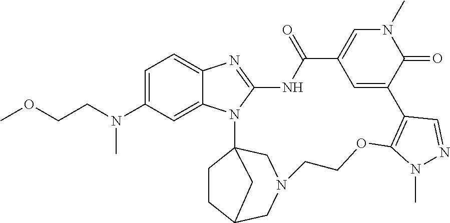 [SMILES: COCCN(C)c1ccc2nc3n(c2c1)C12CCC(CN(CCOc4c(cnn4C)-c4cc(cn(C)c4=O)C(=O)N3)C1)C2] | 587.45 | ¹H NMR (DMSO-d₆, 400 MHz) δ 1.52-1.79 (3H, m), 1.98-2.04 (1H, m), 2.39-2.54 (4H, m), 2.70-2.74 (2H, m), 2.90-2.96 (3H, s), 3.23- 3.30 (4H, m), 3.45-3.57 (5H, m), 3.62 (3H, s), 3.63-3.69 (1H, m), 3.72 (3H, s), 4.55 (1H, t), 4.68 (1H, d), 6.68 (1H, dd), 6.99 (1H, d), 7.37 (1H, d), 8.26 (1H, d), 8.42 (1H, s), 8.82 (1H, d), 12.51 (1H, s). |
| | Isomer 1 | | |
| A90B | 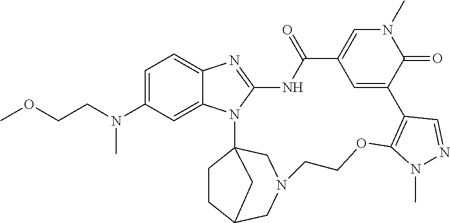 [SMILES: COCCN(C)c1ccc2nc3n(c2c1)C12CCC(CN(CCOc4c(cnn4C)-c4cc(cn(C)c4=O)C(=O)N3)C1)C2] | 587.40 | ¹H NMR (DMSO-d₆, 400 MHz) δ 1.52-1.79 (3H, m), 1.98-2.04 (1H, m), 2.39-2.54 (4H, m), 2.70-2.74 (2H, m), 2.90-2.96 (3H, s), 3.23- 3.30 (4H, m), 3.45-3.57 (5H, m), 3.62 (3H, s), 3.63-3.69 (1H, m), 3.72 (3H, s), 4.55 (1H, t), 4.68 (1H, d), 6.68 (1H, dd), 6.99 (1H, d), 7.37 (1H, d), 8.26 (1H, d), 8.42 (1H, s), 8.82 (1H, d), 12.51 (1H, s). |
| | Isomer 2 | | |
| A91A | 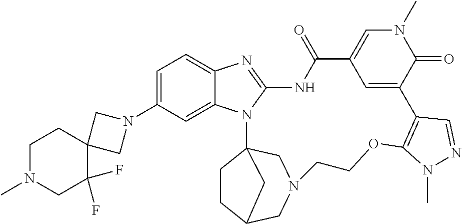 [SMILES: CN1CCC2(CN(c3ccc4nc5n(c4c3)C34CCC(CN(CCOc6c(cnn6C)-c6cc(cn(C)c6=O)C(=O)N5)C3)C4)C2)C(F)(F)C1] | 674.40 | ¹H NMR (DMSO-d₆, 400 MHz) δ 1.50-1.81 (3H, m), 1.98-2.02 (3H, m), 2.23 (3H, s), 2.41-2.48 (4H, m), 2.39-2.54 (4H, m), 2.63-2.76 (2H, m), 3.20-3.40 (2H, m), 3.62 (3H, s), 3.63-3.69 (3H, m), 3.72 (3H, s), 3.87 (2H, t), 4.55 (1H, t), 4.67 (1H, d), 6.36 (1H, dd), 6.68 (1H, d), 7.40 (1H, d), 8.26 (1H, d), 8.41 (1H, s), 8.81 (1H, d), 12.55 (1H, s). |
| | Isomer 1 | | |
| A91B | 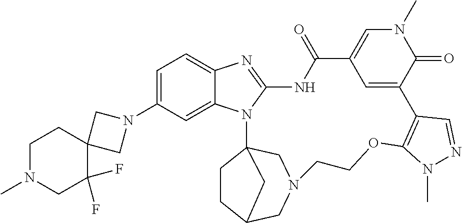 [SMILES: CN1CCC2(CN(c3ccc4nc5n(c4c3)C34CCC(CN(CCOc6c(cnn6C)-c6cc(cn(C)c6=O)C(=O)N5)C3)C4)C2)C(F)(F)C1] | 674.40 | ¹H NMR (DMSO-d₆, 400 MHz) δ 1.50-1.81 (3H, m), 1.98-2.02 (3H, m), 2.23 (3H, s), 2.41-2.48 (4H, m), 2.39-2.54 (4H, m), 2.63-2.76 (2H, m), 3.20-3.40 (2H, m), 3.62 (3H, s), 3.63-3.69 (3H, m), 3.72 (3H, s), 3.87 (2H, t), 4.55 (1H, t), 4.67 (1H, d), 6.36 (1H, dd), 6.68 (1H, d), 7.40 (1H, d), 8.26 (1H, d), 8.41 (1H, s), 8.81 (1H, d), 12.55 (1H, s). |
| | Isomer 2 | | |
| A92A | 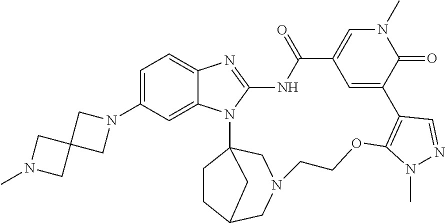 [SMILES: CN1CC2(C1)CN(c1ccc3nc4n(c3c1)C13CCC(CN(CCOc5c(cnn5C)-c5cc(cn(C)c5=O)C(=O)N4)C1)C3)C2] | 610.40 | ¹H NMR (DMSO-d₆, 400 MHz) δ 1.53-1.77 (3H, m), 1.98 (1H, d), 2.20 (3H, s), 2.39-2.47 (2H, m), 2.51-2.54 (1H, m), 2.63-2.76 (2H, m), 3.17 (2H, d), 3.22-3.29 (5H, m), 3.62 (3H, s), 3.63-3.69 (1H, m), 3.72 (3H, s), 3.86 (4H, d), 4.55 (1H, t), 4.66 (1H, d), 6.33 (1H, dd), 6.64 (1H, s), 7.37 (1H, d), 8.26 (1H, d), 8.41 (1H, s), 8.81 (1H, d), 12.53 (1H, s). |
| | Isomer 1 | | |
| A92B | 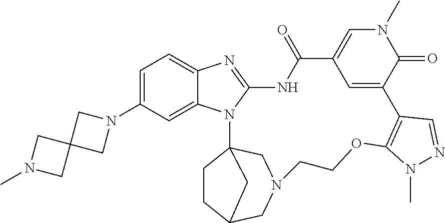 [SMILES: CN1CC2(C1)CN(c1ccc3nc4n(c3c1)C13CCC(CN(CCOc5c(cnn5C)-c5cc(cn(C)c5=O)C(=O)N4)C1)C3)C2] | 610.35 | ¹H NMR (DMSO-d₆, 400 MHz) δ 1.53-1.77 (3H, m), 1.98 (1H, d), 2.20 (3H, s), 2.39-2.47 (2H, m), 2.51-2.54 (1H, m), 2.63-2.76 (2H, m), 3.17 (2H, d), 3.22-3.29 (5H, m), 3.62 (3H, s), 3.63-3.69 (1H, m), 3.72 (3H, s), 3.86 (4H, d), 4.55 (1H, t), 4.66 (1H, d), 6.33 (1H, dd), 6.64 (1H, s), 7.37 (1H, d), 8.26 (1H, d), 8.41 (1H, s), 8.81 (1H, d), 12.53 (1H, s). |
| | Isomer 2 | | |
| A95A | 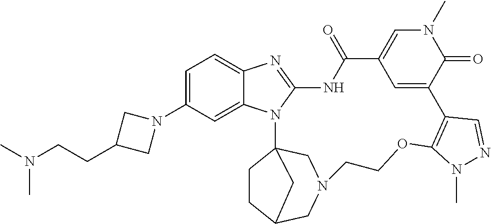 [SMILES: CN(C)CCC1CN(c2ccc3nc4n(c3c2)C23CCC(CN(CCOc5c(cnn5C)-c5cc(cn(C)c5=O)C(=O)N4)C2)C3)C1] | 626.45 | ¹H NMR (DMSO-d₆, 400 MHz) δ 1.47-1.71 (3H, m), 1.95-2.02 (2H, m), 2.47 (6H, s), 2.60-2.66 (2H, m), 2.70-2.76 (1H, m), 2.80-2.84 (1H, m), 3.03-3.15 (4H, m), 3.17- 3.26 (4H, m), 3.56 (2H, t), 3.61 (3H, s), 3.63-3.70 (2H, m), 3.72 (3H, s), 4.54 (1H, t), 4.69 (1H, d), 5.69 (1H, s), 6.51 (1H, d), 6.93 (1H, s), 7.30 (1H, s), 8.24 (1H, s), 8.41 (1H, s), 8.80 (1H, s). |
| | Isomer 1 | | |
| A95B | 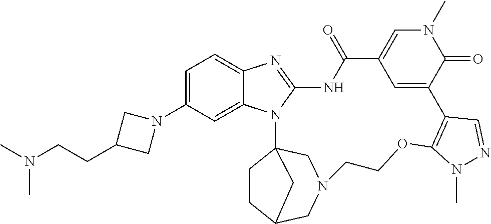 [SMILES: CN(C)CCC1CN(c2ccc3nc4n(c3c2)C23CCC(CN(CCOc5c(cnn5C)-c5cc(cn(C)c5=O)C(=O)N4)C2)C3)C1] | 626.20 | ¹H NMR (DMSO-d₆, 400 MHz) δ 1.47-1.71 (3H, m), 1.95-2.02 (2H, m), 2.47 (6H, s), 2.60-2.66 (2H, m), 2.70-2.76 (1H, m), 2.80-2.84 (1H, m), 3.03-3.15 (4H, m), 3.17- 3.26 (4H, m), 3.56 (2H, t), 3.61 (3H, s), 3.63-3.70 (2H, m), 3.72 (3H, s), 4.54 (1H, t), 4.69 (1H, d), 5.69 (1H, s), 6.48 (1H, d), 6.91 (1H, s), 7.24 (1H, s), 8.20 (1H, s), 8.40 (1H, s), 8.79 (1H, s). |
| | Isomer 2 | | |
| A97A | 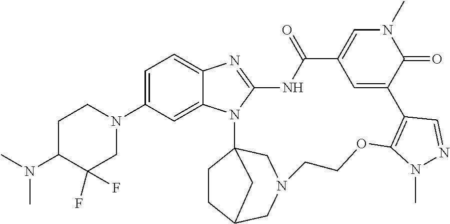 [SMILES: CN(C)C1CCN(c2ccc3nc4n(c3c2)C23CCC(CN(CCOc5c(cnn5C)-c5cc(cn(C)c5=O)C(=O)N4)C2)C3)CC1(F)F] | 662.35 | ¹H NMR (DMSO-d₆, 400 MHz) δ 1.53-1.77 (3H, m), 1.86-1.95 (2H, m), 1.98-2.04 (1H, m), 2.40 (6H, s), 2.42-2.62 (5H, m), 2.62-2.74 (2H, m), 2.79-3.10 (3H, m), 3.19- 3.30 (2H, d), 3.62 (3H, s), 3.63- 3.71 (2H, m), 3.72 (3H, s), 4.55 (1H, t), 4.67 (1H, d), 6.88-6.94 (1H, m), 7.21 (1H, d), 7.42 (1H, d), 8.27 (1H, d), 8.42 (1H, s), 8.82 (1H, d), 12.62 (1H, s). |
| | Isomer 1 | | |
| A97B | 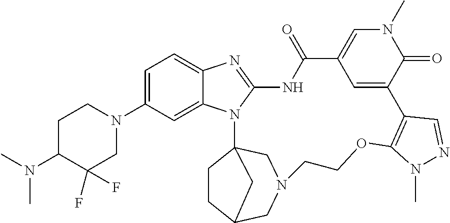 [SMILES: CN(C)C1CCN(c2ccc3nc4n(c3c2)C23CCC(CN(CCOc5c(cnn5C)-c5cc(cn(C)c5=O)C(=O)N4)C2)C3)CC1(F)F] | 662.35 | ¹H NMR (DMSO-d₆, 400 MHz) δ 1.53-1.77 (3H, m), 1.86-1.95 (2H, m), 1.98-2.04 (1H, m), 2.40 (6H, s), 2.42-2.62 (5H, m), 2.62-2.74 (2H, m), 2.79-3.10 (3H, m), 3.19- 3.30 (2H, d), 3.62 (3H, s), 3.63- 3.71 (2H, m), 3.72 (3H, s), 4.55 (1H, t), 4.67 (1H, d), 6.88-6.94 (1H, m), 7.21 (1H, d), 7.42 (1H, d), 8.27 (1H, d), 8.42 (1H, s), 8.82 (1H, d), 12.61 (1H, s). |
| | Isomer 2 | | |
| A98A | 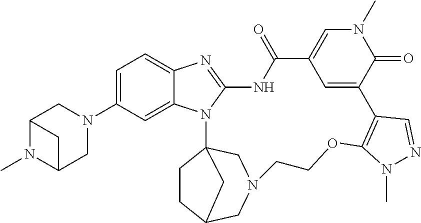 [SMILES: CN1C2CC1CN(c1ccc3nc4n(c3c1)C13CCC(CN(CCOc5c(cnn5C)-c5cc(cn(C)c5=O)C(=O)N4)C1)C3)C2] | 610.40 | ¹H NMR (DMSO-d₆, 400 MHz) δ 1.53-1.78 (4H, m), 2.00-2.09 (4H, m), 2.30-2.60 (6H, m), 2.72-2.79 (2H, m), 3.21-3.51 (5H, m), 3.55- 3.70 (3H, m), 3.61 (3H, s), 3.72 (3H, s), 4.56 (1H, t), 4.70 (1H, d), 6.68 (1H, d), 6.98 (1H, s), 7.43 (1H, d), 8.26 (1H, d), 8.42 (1H, s), 8.83 (1H, d), 12.53 (1H, s). |
| | Isomer 1 | | |
| A98B | 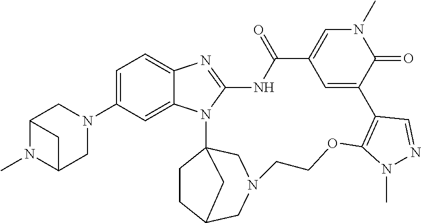 [SMILES: CN1C2CC1CN(c1ccc3nc4n(c3c1)C13CCC(CN(CCOc5c(cnn5C)-c5cc(cn(C)c5=O)C(=O)N4)C1)C3)C2] | 610.40 | ¹H NMR (DMSO-d₆, 400 MHz) δ 1.53-1.78 (4H, m), 2.00-2.09 (4H, m), 2.30-2.60 (6H, m), 2.72-2.79 (2H, m), 3.21-3.51 (5H, m), 3.55- 3.70 (3H, m), 3.61 (3H, s), 3.72 (3H, s), 4.56 (1H, t), 4.70 (1H, d), 6.68 (1H, d), 6.98 (1H, s), 7.43 (1H, d), 8.26 (1H, d), 8.42 (1H, s), 8.83 (1H, d), 12.53 (1H, s). |
| | Isomer 2 | | |
| A99A | 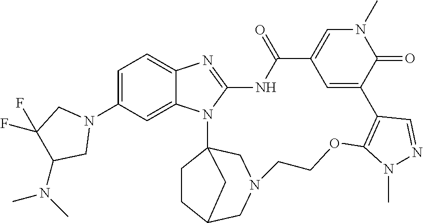 [SMILES: CN(C)C1CN(c2ccc3nc4n(c3c2)C23CCC(CN(CCOc5c(cnn5C)-c5cc(cn(C)c5=O)C(=O)N4)C2)C3)CC1(F)F] | 648.30 | ¹H NMR (DMSO-d₆, 400 MHz) δ 1.53-1.77 (3H, m), 1.97-2.03 (1H, m), 2.34 (6H, s), 2.40-2.66 (5H, m), 2.70-2.76 (2H, m), 3.16-3.28 (4H, m), 3.62 (3H, s), 3.63-3.69 (1H, m), 3.72 (3H, s), 3.73-3.94 (2H, m), 4.55 (1H, t), 4.68 (1H, d), 6.58 (1H, d), 6.83 (1H, d), 7.42 (1H, d), 8.27 (1H, d), 8.42 (1H, s), 8.82 (1H, d), 12.55 (1H, s). |
| | Isomer 1 | | |
| A99B | 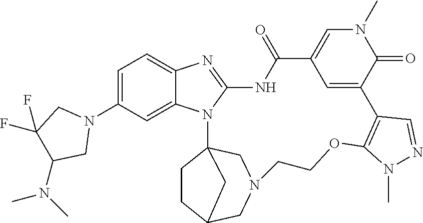 [SMILES: CN(C)C1CN(c2ccc3nc4n(c3c2)C23CCC(CN(CCOc5c(cnn5C)-c5cc(cn(C)c5=O)C(=O)N4)C2)C3)CC1(F)F] | 648.35 | ¹H NMR (DMSO-d₆, 400 MHz) δ 1.53-1.77 (3H, m), 1.97-2.03 (1H, m), 2.34 (6H, s), 2.40-2.66 (5H, m), 2.70-2.76 (2H, m), 3.16-3.28 (4H, m), 3.62 (3H, s), 3.63-3.69 (1H, m), 3.72 (3H, s), 3.73-3.94 (2H, m), 4.55 (1H, t), 4.68 (1H, d), 6.58 (1H, d), 6.83 (1H, d), 7.42 (1H, d), 8.27 (1H, d), 8.42 (1H, s), 8.82 (1H, d), 12.56 (1H, s). |
| | Isomer 2 | | |
| A104A | 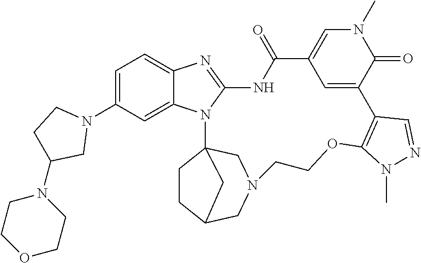 [SMILES: Cn1ncc2c1OCCN1CC3CCC(C3)(C1)n1c(nc3ccc(N4CCC(N5CCOCC5)C4)cc31)NC(=O)c1cc-2c(=O)n(C)c1] | 654.40 | ¹H NMR (DMSO-d₆, 400 MHz) δ 1.52-1.88 (4H, m), 2.00 (1H, d), 2.19 (1H, s), 2.38-2.58 (9H, m), 2.65-2.75 (3H, m), 2.87-3.31 (3H, m), 3.35-3.51 (2H, m), 3.59-3.68 (8H, m), 3.72 (3H, s), 4.55 (1H, t), 4.66 (1H, d), 6.49 (1H, d), 6.76 (1H, s), 7.38 (1H, d), 8.26 (1H, d), 8.42 (1H, s), 8.82 (1H, d), 12.51 (1H, s). |
| | Isomer 1 | | |
| A104B | 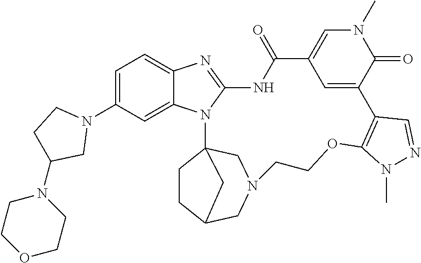 [SMILES: Cn1ncc2c1OCCN1CC3CCC(C3)(C1)n1c(nc3ccc(N4CCC(N5CCOCC5)C4)cc31)NC(=O)c1cc-2c(=O)n(C)c1] | 654.35 | ¹H NMR (DMSO-d₆, 400 MHz) δ 1.52-1.88 (4H, m), 2.00 (1H, d), 2.19 (1H, s), 2.38-2.58 (9H, m), 2.65-2.75 (3H, m), 2.87-3.31 (3H, m), 3.35-3.51 (2H, m), 3.59-3.68 (8H, m), 3.72 (3H, s), 4.55 (1H, t), 4.66 (1H, d), 6.49 (1H, d), 6.76 (1H, s), 7.38 (1H, d), 8.26 (1H, d), 8.42 (1H, s), 8.82 (1H, d), 12.51 (1H, s). |
| | Isomer 2 | | |
| A105A | 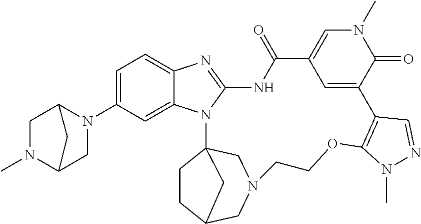 [SMILES: CN1CC2CC1CN2c1ccc2nc3n(c2c1)C12CCC(CN(CCOc4c(cnn4C)-c4cc(cn(C)c4=O)C(=O)N3)C1)C2] | 610.35 | ¹H NMR (DMSO-d₆, 400 MHz) δ 1.57-1.71 (4H, m), 1.76 -1.90(1H, m), 2.02 (1H, d), 2.25 (3H, s), 2.53- 2.63 (2H, m), 2.68-2.79 (4H, m), 2.82-2.91 (2H, m), 3.19-3.31 (2H, m), 3.52-3.62 (2H, m), 3.62 (3H, s), 3.63-3.71 (1H, m), 3.72 (3H, s), 4.31 (1H, s), 4.55 (1H, t), 4.66 (1H, d), 6.51 (1H, dd), 6.78 (1H, d), 7.36 (1H, d), 8.26 (1H, d), 8.42 (1H, s), 8.82 (1H, d), 12.50 (1H, s). |
| | Isomer 1 | | |
| A105B | 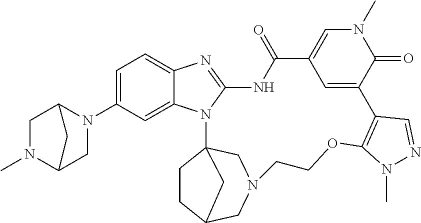 [SMILES: CN1CC2CC1CN2c1ccc2nc3n(c2c1)C12CCC(CN(CCOc4c(cnn4C)-c4cc(cn(C)c4=O)C(=O)N3)C1)C2] | 610.35 | ¹H NMR (DMSO-d₆, 400 MHz) δ 1.57-1.71 (4H, m), 1.76 -1.90(1H, m), 2.02 (1H, d), 2.25 (3H, s), 2.53- 2.63 (2H, m), 2.68-2.79 (4H, m), 2.82-2.91 (2H, m), 3.19-3.31 (2H, m), 3.52-3.62 (2H, m), 3.62 (3H, s), 3.63-3.71 (1H, m), 3.72 (3H, s), 4.31 (1H, s), 4.55 (1H, t), 4.66 (1H, d), 6.52 (1H, dd), 6.76 (1H, d), 7.35 (1H, d), 8.26 (1H, d), 8.42 (1H, s), 8.82 (1H, d), 12.50 (1H, s). |
| | Isomer 2 | | |
| A106A | 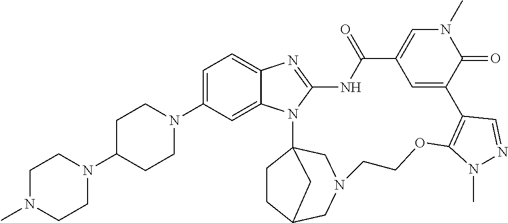 [SMILES: CN1CCN(C2CCN(c3ccc4nc5n(c4c3)C34CCC(CN(CCOc6c(cnn6C)-c6cc(cn(C)c6=O)C(=O)N5)C3)C4)CC2)CC1] | 681.50 | ¹H NMR (DMSO-d₆, 400 MHz) δ 1.53-1.77 (7H, m), 2.00-2.07 (3H, m), 2.43-2.62 (8H, m), 2.67-2.88 (10H, m), 3.14-3.22 (1H, m), 3.47- 3.66 (4H, m), 3.70 (3H, s), 3.78 (3H, s), 4.71 (1H, t), 4.78 (1H, d), 6.89 (1H, dd), 7.18 (1H, d), 7.24 (1H, s), 8.18 (1H, d), 8.62 (1H, s), 8.91 (1H, d), 12.33 (1H, s). |
| | Isomer 1 | | |
| A106B | 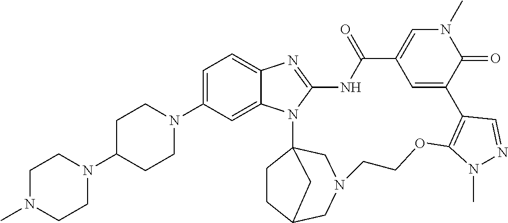 [SMILES: CN1CCN(C2CCN(c3ccc4nc5n(c4c3)C34CCC(CN(CCOc6c(cnn6C)-c6cc(cn(C)c6=O)C(=O)N5)C3)C4)CC2)CC1] | 681.50 | ¹H NMR (DMSO-d₆, 400 MHz) δ 1.53-1.77 (7H, m), 2.00-2.07 (3H, m), 2.43-2.62 (8H, m), 2.67-2.88 (10H, m), 3.14-3.22 (1H, m), 3.47- 3.66 (4H, m), 3.70 (3H, s), 3.78 (3H, s), 4.71 (1H, t), 4.78 (1H, d), 6.89 (1H, dd), 7.18 (1H, d), 7.24 (1H, s), 8.18 (1H, d), 8.62 (1H, s), 8.91 (1H, d), 12.32 (1H, s). |
| | Isomer 2 | | |
| A107A | 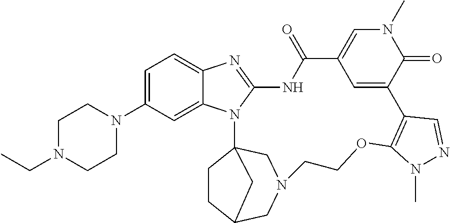 [SMILES: CCN1CCN(c2ccc3nc4n(c3c2)C23CCC(CN(CCOc5c(cnn5C)-c5cc(cn(C)c5=O)C(=O)N4)C2)C3)CC1] | 612.20 | ¹H NMR (CDCl₃, 400 MHz) δ 1.40-1.53 (3H, m), 1.65-1.75 (1H, m), 1.76-1.90 (2H, m), 2.02-2.10 (1H, m), 2.46-2.50 (3H, m), 2.61- 2.67 (1H, m), 2.74-2.80 (2H, m), 2.98-3.24 (6H, m), 3.43-3.59 (7H, m), 3.70 (3H, s), 3.79 (3H, s), 4.71 (1H, t), 4.78 (1H, d), 6.89 (1H, dd), 7.21 (1H, d), 7.22-7.25 (1H, m), 8.18 (1H, d), 8.62 (1H, s), 8.91 (1H, d), 12.38 (1H, s). |
| | Isomer 1 | | |
| A107B | 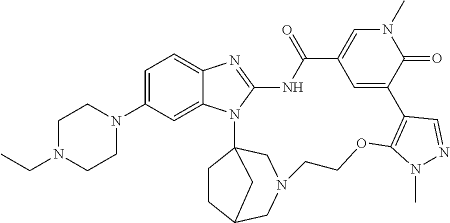 [SMILES: CCN1CCN(c2ccc3nc4n(c3c2)C23CCC(CN(CCOc5c(cnn5C)-c5cc(cn(C)c5=O)C(=O)N4)C2)C3)CC1] | 612.20 | ¹H NMR (CDCl₃, 400 MHz) δ 1.40-1.53 (3H, m), 1.65-1.75 (1H, m), 1.80-1.88 (2H, m), 2.02-2.10 (1H, m), 2.46-2.50 (3H, m), 2.61- 2.67 (1H, m), 2.74-2.80 (2H, m), 2.87-3.25 (7H, m), 3.38-3.60 (6H, m), 3.70 (3H, s), 3.79 (3H, s), 4.71 (1H, t), 4.78 (1H, d), 6.89 (1H, dd), 7.20 (1H, d), 7.22-7.25 (1H, m), 8.18 (1H, d), 8.62 (1H, s), 8.91 (1H, d), 12.37 (1H, s). |
| | Isomer 2 | | |
| A108A | 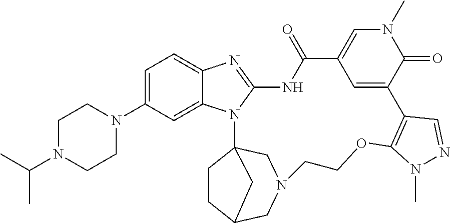 [SMILES: CC(C)N1CCN(c2ccc3nc4n(c3c2)C23CCC(CN(CCOc5c(cnn5C)-c5cc(cn(C)c5=O)C(=O)N4)C2)C3)CC1] | 626.45 | ¹H NMR (CDCl₃, 400 MHz) δ 1.19 (6H, br s), 1.53-1.77 (3H, m), 2.05 (1H, d), 2.42-2.52 (3H, m), 2.60- 2.66 (1H, m), 2.72-2.81 (2H, m), 2.91-3.01 (4H, m), 3.12-3.25 (2H, m), 3.36 (3H, s), 3.44-3.64 (2H, m), 3.70 (3H, s), 3.79 (3H, s), 4.71 (1H, t), 4.78 (1H, d), 6.89 (1H, dd), 7.19 (1H, d), 7.22-7.25 (1H, m), 8.18 (1H, d), 8.62 (1H, s), 8.91 (1H, d), 12.35 (1H, s). |
| | Isomer 1 | | |
| A108B | 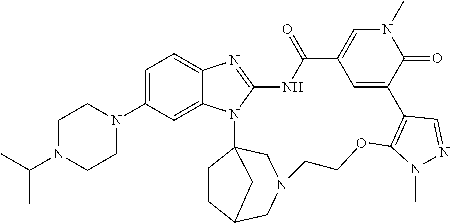 [SMILES: CC(C)N1CCN(c2ccc3nc4n(c3c2)C23CCC(CN(CCOc5c(cnn5C)-c5cc(cn(C)c5=O)C(=O)N4)C2)C3)CC1] | 626.45 | ¹H NMR (CDCl₃, 400 MHz) δ 1.19 (6H, br s), 1.53-1.77 (3H, m), 2.05 (1H, d), 2.41-2.56 (3H, m), 2.60- 2.66 (1H, m), 2.72-2.81 (2H, m), 2.91-3.01 (4H, m), 3.12-3.25 (2H, m), 3.36 (3H, s), 3.44-3.64 (2H, m), 3.70 (3H, s), 3.79 (3H, s), 4.71 (1H, t), 4.78 (1H, d), 6.89 (1H, dd), 7.18 (1H, d), 7.22-7.25 (1H, m), 8.18 (1H, d), 8.62 (1H, s), 8.91 (1H, d), 12.33 (1H, s). |
| | Isomer 2 | | |

### Example A115

### 6-fluoro-15,21-dimethyl-5-(4-methylpiperazin-1-yl)-23-oxa-2,9,11,15,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,17(34),18(22),19-octaene-12,16-dione

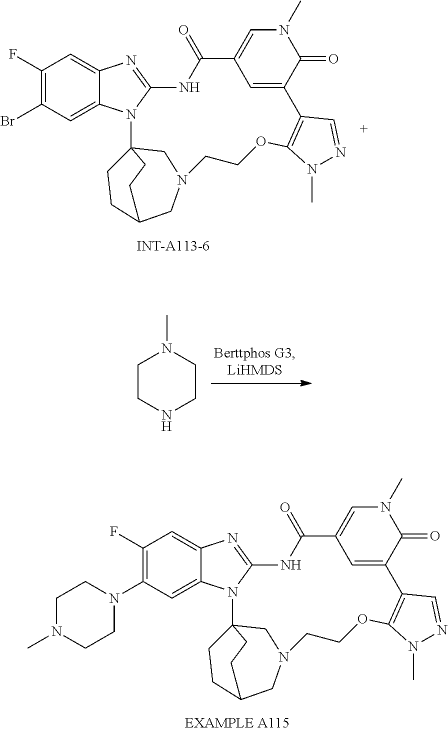

To a stirred mixture of 5-bromo-6-fluoro-15,21-dimethyl-23-oxa-2,9,11,15,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,17(34),18(22),19-octaene-12,16-dione (INT-A113-6, 50 mg, 0.08 mmol, 1 equiv), BrettPhos Pd G₃ (23 mg, 0.03 mmol, 0.3 equiv) and 1-methylpiperazine (25 mg, 0.25 mmol, 3 equiv) in 1,4-dioxane (4 mL) was added LiHMDS (0.82 mL, 0.82 mmol, 10 equiv) slowly at room temperature under nitrogen atmosphere. The resulting mixture was stirred for 30 min at 60 °C and then cooled to room temperature. The reaction was quenched with saturated NH₄Cl (aq.) (80 mL) and the mixture was extracted with dichloromethane (3×80 mL). The combined organic layers were washed with brine (3×50 mL), dried over anhydrous Na₂SO₄, and concentrated under reduced pressure. The residue was purified by prep-TLC and eluted with dichloromethane/methanol (12/1). The product was further purified by prep-HPLC with XBridge prep OBD C18 column using water containing 10 mmol/L NH₄HCO₃ and 0.10% NH₃·H₂O and acetonitrile as mobile phase. Fractions containing the desired compound were evaporated to dryness to give 0.8 mg of 6-fluoro-15,21-dimethyl-5-(4-methylpiperazin-1-yl)-23-oxa-2,9,11,15,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,17(34),18(22),19-octaene-12,16-dione (Example A115) as a white solid (2%). LCMS: m/z (ESI), [M+H]⁺=630.40. ¹H NMR (CD₃OD, 400 MHz) δ 2.02-2.07 (4H, m), 2.08-2.19 (4H, m), 2.40 (3H, s), 2.62-2.78 (4H, m), 2.85-2.97 (4H, m), 3.03-3.13 (3H, m), 3.43-3.55 (4H, m), 3.69 (3H, s), 3.79 (3H, s), 4.31 (2H, t), 7.23 (1H, s), 7.51 (1H, d), 8.33 (1H, s), 8.34 (1H, s), 9.27 (1H, s).

### Example A116

### 6-fluoro-15,21-dimethyl-23-oxa-2,9,11,15,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,17(34),18(22),19-octaene-12,16-dione

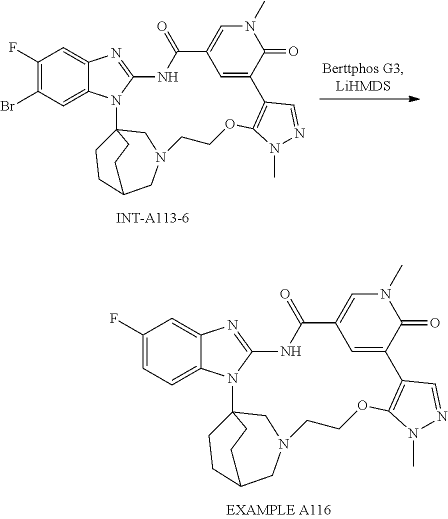 [SMILES: Cn1ncc2c1OCCN1CC3CCC(CC3)(C1)n1c(nc3cc(F)c(Br)cc31)NC(=O)c1cc-2c(=O)n(C)c1 ; Cn1ncc2c1OCCN1CC3CCC(CC3)(C1)n1c(nc3cc(F)ccc31)NC(=O)c1cc-2c(=O)n(C)c1]

To a stirred mixture of 5-bromo-6-fluoro-15,21-dimethyl-23-oxa-2,9,11,15,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,17(34),18(22),19-octaene-12,16-dione (INT-A113-6, 20 mg, 0.03 mmol, 1 equiv) and BrettPhos Pd G₃ (9 mg, 0.01 mmol, 0.3 equiv) in 1,4-dioxane (2 mL) was added LiHMDS (0.33 mL, 0.33 mmol, 10 equiv) slowly at room temperature under nitrogen atmosphere. The resulting mixture was stirred for 30 min at 60 °C and cooled to room temperature. The reaction was quenched with saturated NH₄Cl (aq.) (50 mL). The mixture was extracted with dichloromethane (3×50 mL). The combined organic layers were washed with brine (3×20 mL), dried over anhydrous Na₂SO₄, and concentrated under reduced pressure. The residue was purified by prep-TLC and eluted with dichloromethane/methanol (22/1). The product was further purified by prep-HPLC with XBridge Shield RP18 OBD column using water containing 10 mmol/L NH₄HCO₃ and acetonitrile as mobile phase. Fractions containing the desired compound were evaporated to dryness to give 0.9 mg of 6-fluoro-15,21-dimethyl-23-oxa-2,9,11,15,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,17(34),18(22),19-octaene-12,16-dione (Example A116) as a white solid (5%). LCMS: m/z (EST), [M+H]⁺=532.15. ¹H NMR (DMSO-d₆, 400 MHz) δ 1.88-1.94 (4H, m), 1.96-2.13 (3H, m), 2.79 (2H, d), 2.86 (2H, t), 3.39-3.42 (4H, m), 3.62 (3H, s), 3.74 (3H, s), 4.28 (2H, t), 6.91-7.01 (1H, m), 7.45 (1H, dd), 7.87 (1H, dd), 8.27-8.35 (2H, m), 9.10 (1H, d), 13.34 (1H, s).

### Example A117A, Example A117B, Example A118A and Example A118B

### 6-fluoro-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, Isomer 1 (Example A117A) and Isomer 2 (Example A117B); 6-fluoro-15,21-dimethyl-5-(morpholin-4-yl)-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, Isomer 1 (Example A118A) and Isomer 2 (Example A118B)

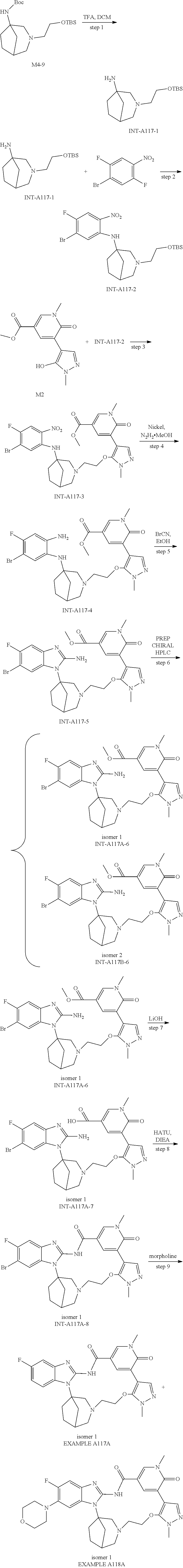

Step 1. Preparation of 3-(2-((tert-butyldimethylsilyl)oxy)ethyl)-3-azabicyclo[3.2.1]octan-1-amine (INT-A117-1). A mixture of tert-butyl N-(3-{2-[(tert-butyldimethylsilyl)oxy]ethyl}-3-azabicyclo[3.2.1]octan-1-yl)carbamate (M4-9, 10 g, 26 mmol, 1 equiv) in trifluoroacetic acid (35 mL) and dichloromethane (100 mL) was stirred for 2 h at room temperature. The resulting mixture was concentrated under reduced pressure. The residue was treated with saturated NaHCO₃ (aq.) (100 mL) and extracted with dichloromethane (3×100 mL). The combined organic layers were washed with brine (3×200 mL), dried over anhydrous Na₂SO₄, and concentrated under reduced pressure to give 7.3 g of 3-(2-((tert-butyldimethylsilyl)oxy)ethyl)-3-azabicyclo[3.2.1]octan-1-amine (INT-A117-1) as a yellow solid (98%). The crude product was used without purification. LCMS: m/z (ESI), [M+H]⁺=285.15.

Step 2. Preparation of 2-{1-[(5-bromo-4-fluoro-2-nitrophenyl)amino]-3-azabicyclo[3.2.1]octan-3-yl}ethanol (INT-A117-2). A mixture of 3-{2-[(tert-butyldimethylsilyl)oxy]ethyl}-3-azabicyclo[3.2.1]octan-1-amine (2 g, 7.03 mmol, 1 equiv), triethylamine (2.84 g, 28.11 mmol, 4 equiv) and 1-bromo-2,5-difluoro-4-nitrobenzene (5.01 g, 21.08 mmol, 3 equiv) in DMSO (30 mL) was stirred for 3 h at 100 °C. The mixture was cooled to room temperature. The reaction was quenched with water (50 mL) and the mixture was extracted with ethyl acetate (3×50 mL). The combined organic layers were washed with brine (3×100 mL), dried over anhydrous Na₂SO₄, and concentrated under reduced pressure. The residue was purified on a silica gel column and eluted with ethyl acetate/petroleum ether (1/1) to give 1.5 g of 2-{1-[(5-bromo-4-fluoro-2-nitrophenyl)amino]-3-azabicyclo[3.2.1]octan-3-yl}ethanol (INT-A117-2) as an orange oil (55%). LCMS: m/z (ESI), [M+H]⁺=387.95.

Step 3. Preparation of methyl 5-[5-(2-{l-[(5-bromo-4-fluoro-2-nitrophenyl)amino]-3-azabicyclo[3.2.1]octan-3-yl}ethoxy)-1-methylpyrazol-4-yl]-1-methyl-6-oxopyridine-3-carboxylate (INT-A117-3). A mixture of 2-{1-[(5-bromo-4-fluoro-2-nitrophenyl)amino]-3-azabicyclo[3.2.1]octan-3-yl}ethanol (700 mg, 1.80 mmol, 1 equiv), methyl 5-(5-hydroxy-1-methylpyrazol-4-yl)-1-methyl-6-oxopyridine-3-carboxylate (M2, 712 mg, 2.70 mmol, 1.5 equiv) and 2-(tributylphosphoranylidene)acetonitrile (2.61 g, 10.82 mmol, 6 equiv) in toluene (20 mL) was stirred for 2 h at 100 °C under nitrogen atmosphere. The mixture was cooled to room temperature and concentrated under reduced pressure. The residue was treated with water (50 mL) and the mixture was extracted with CH₂Cl₂ (3×50 mL). The combined organic layers were washed with brine (3×100 mL), dried over anhydrous Na₂SO₄, and concentrated under reduced pressure. The residue was purified by prep-TLC and eluted with CH₂Cl₂/methanol (25/1) to give 700 mg of methyl 5-[5-(2-{1-[(5-bromo-4-fluoro-2-nitrophenyl)amino]-3-azabicyclo[3.2.1]octan-3-yl}ethoxy)-1-methylpyrazol-4-yl]-1-methyl-6-oxopyridine-3-carboxylate (INT-A117-3) as a yellow solid (61%). LCMS: m/z (ESI), [M+H]⁺=633.35. ¹H NMR (DMSO-d₆, 400 MHz) δ 1.53-1.74 (4H, m), 1.75-1.87 (1H, m), 1.93 (1H, s), 2.16 (1H, d), 2.21 (2H, d), 2.70 (1H, d), 2.76-2.86 (2H, m), 3.16 (1H, d), 3.57 (3H, s), 3.76 (6H, d), 4.09 (2H, t), 7.26 (1H, d), 7.97 (1H, s), 8.04-8.11 (2H, m), 8.17 (1H, s), 8.41 (1H, d).

Step 4. Preparation of methyl 5-[5-(2-{1-[(2-amino-5-bromo-4-fluorophenyl)amino]-3-azabicyclo[3.2.1]octan-3-yl}ethoxy)-1-methylpyrazol-4-yl]-1-methyl-6-oxopyridine-3-carboxylate (INT-A117-4). To a stirred mixture of methyl 5-[5-(2-{1-[(5-bromo-4-fluoro-2-nitrophenyl)amino]-3-azabicyclo[3.2.1]octan-3-yl}ethoxy)-1-methylpyrazol-4-yl]-1-methyl-6-oxopyridine-3-carboxylate (680 mg, 1.07 mmol, 1 equiv) and Raney Nickel (184 mg, 2.15 mmol, 2 equiv) in methanol (10 mL) was added hydrazine hydrate (107 mg, 2.15 mmol, 2 equiv) at room temperature. The resulting mixture was stirred for 1 h at room temperature and filtered. The filter cake was washed with methanol (3×10 mL). The filtrate was concentrated under reduced pressure to give 600 mg of methyl 5-[5-(2-{1-[(2-amino-5-bromo-4-fluorophenyl)aamino]-3-azabicyclo[3.2.1]octan-3-yl}ethoxy)-1-methylpyrazol-4-yl]-1-methyl-6-oxopyridine-3-carboxylate (INT-A117-4) as a white solid (99%). The crude product was used for the next reaction without purification. LCMS: m/z (ESI), [M+H]⁺=603.10.

Step 5. Preparation of methyl 5-(5-{2-[1-(2-amino-6-bromo-5-fluoro-1,3-benzodiazol-1-yl)-3-azabicyclo[3.2.1]octan-3-yl]ethoxy}-1-methylpyrazol-4-yl)-1-methyl-6-oxopyridine-3-carboxylate (INT-A117-5). A mixture of methyl 5-[5-(2-{l-[(2-amino-5-bromo-4-fluorophenyl)amino]-3-azabicyclo[3.2.1]octan-3-yl}ethoxy)-1-methylpyrazol-4-yl]-1-methyl-6-oxopyridine-3-carboxylate (550 mg, 0.91 mmol, 1 equiv) and BrCN (193 mg, 1.82 mmol, 2 equiv) in ethanol (10 mL) was stirred overnight at room temperature under nitrogen atmosphere. The reaction mixture was concentrated under vacuum. The residue was purified by trituration with N,N-dimethylformamide (10 mL) to give 280 mg of methyl 5-(5-{2-[1-(2-amino-6-bromo-5-fluoro-1,3-benzodiazol-1-yl)-3-azabicyclo[3.2.1]octan-3-yl]ethoxy}-1-methylpyrazol-4-yl)-1-methyl-6-oxopyridine-3-carboxylate (INT-A117-5) as a yellow solid (48%). LCMS: m/z (ESI), [M+H]⁺=628.10.

Step 6. Preparation of methyl 5-(5-{2-[1-(2-amino-6-bromo-5-fluoro-1,3-benzodiazol-1-yl)-3-azabicyclo[3.2.1]octan-3-yl]ethoxy}-1-methylpyrazol-4-yl)-1-methyl-6-oxopyridine-3-carboxylate (isomer 1, INT-A117A-6 and isomer 2, INT-A117B-6). The racemic mixture of methyl 5-(5-{2-[1-(2-amino-6-bromo-5-fluoro-1,3-benzodiazol-1-yl)-3-azabicyclo[3.2.1]octan-3-yl]ethoxy}-1-methylpyrazol-4-yl)-1-methyl-6-oxopyridine-3-carboxylate (200 mg) was separated by prep-chiral-HPLC with the column of CHIRALPAK IG (2×25 cm, 5 μm) eluting with mobile phase A of hexane (containing 0.1% diethylamine) and mobile phase B of ethanol/CH₂Cl₂ (1/1) to give methyl 5-(5-{2-[1-(2-amino-6-bromo-5-fluoro-1,3-benzodiazol-1-yl)-3-azabicyclo[3.2.1]octan-3-yl]ethoxy}-1-methylpyrazol-4-yl)-1-methyl-6-oxopyridine-3-carboxylate, 90 mg of isomer 1 (INT-A117A-6, 45%) and 90 mg of isomer 2 (INT-A117B-6, 45%) as an off-white solid.

INT-A117A-6: Chiral-HPLC, Rt=3.619 min.

INT-A117B-6: Chiral-HPLC, Rt=4.508 min.

Step 7. Preparation of 5-(5-{2-[1-(2-amino-6-bromo-5-fluoro-1,3-benzodiazol-1-yl)-3-azabicyclo[3.2.1]octan-3-yl]ethoxy}-1-methylpyrazol-4-yl)-1-methyl-6-oxopyridine-3-carboxylic acid (INT-A117A-7). A mixture of methyl 5-(5-{2-[1-(2-amino-6-bromo-5-fluoro-1,3-benzodiazol-1-yl)-3-azabicyclo[3.2.1]octan-3-yl]ethoxy}-1-methylpyrazol-4-yl)-1-methyl-6-oxopyridine-3-carboxylate (INT-A117A-6, 80 mg, 0.13 mmol, 1 equiv) and LiOH (6 mg, 0.25 mmol, 2 equiv) in THF (2 mL) and H₂O (0.5 mL) was stirred for 2 h at room temperature under nitrogen atmosphere. The mixture was concentrated under reduced pressure to give 65 mg of 5-(5-{2-[(1-(2-amino-6-bromo-5-fluoro-1,3-benzodiazol-1-yl)-3-azabicyclo[3.2.1]octan-3-yl]ethoxy}-1-methylpyrazol-4-yl)-1-methyl-6-oxopyridine-3-carboxylic acid (INT-A117A-7) as a yellow solid (83%). This material was used for the next step without further purification. LCMS: m/z (EST), [M+H]⁺=614.20.

Step 8. Preparation of 5-bromo-6-fluoro-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (INT-A117A-8). A mixture of 5-(5-{2-[1-(2-amino-6-bromo-5-fluoro-1,3-benzodiazol-1-yl)-3-azabicyclo[3.2.1]octan-3-yl]ethoxy}-1-methylpyrazol-4-yl)-1-methyl-6-oxopyridine-3-carboxylic acid (INT-A117A-7, 60 mg, 0.10 mmol, 1 equiv), N,N,N,N-tetramethyl-O-(7-azabenzotriazol-1-yl)uroniumhexafluorophospate (74 mg, 0.20 mmol, 2 equiv) and N,N-diisopropylethylamine (38 mg, 0.30 mmol, 3 equiv) in 1,4-dioxane (6 mL) was stirred for 2 h at 60 °C under nitrogen atmosphere. The mixture was cooled to room temperature. The reaction was quenched with water (20 mL) and the mixture was extracted with CH₂Cl₂ (3×30 mL). The combined organic layers were dried over anhydrous Na₂SO₄ and concentrated under reduced pressure. The residue was purified by prep-TLC and eluted with dichloromethane/methanol (12/1) to give 50 mg of 5-bromo-6-fluoro-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (INT-A117A-8) as a light yellow solid (85%). LCMS: m/z (ESI), [M+H]⁺=594.05.

Step 9. Preparation of 6-fluoro-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (isomer 1, EXAMPLE A117A) and 6-fluoro-15,21-dimethyl-5-(morpholin-4-yl)-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (isomer 1, EXAMPLE A18A).

A mixture of 5-bromo-6-fluoro-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (INT-A117A-8, 50 mg, 0.08 mmol, 1 equiv), morpholine (146 mg, 1.68 mmol, 20 equiv), BrettPhos Pd G₃ (22 mg, 0.03 mmol, 0.3 equiv) and t-BuOK (113 mg, 1.01 mmol, 12 equiv) in 1,4-dioxane (4 mL) was stirred for 2 h at 80 °C under nitrogen atmosphere. The mixture was cooled to room temperature. The reaction was quenched with water (20 mL) and the mixture was extracted with CH₂Cl₂ (3×30 mL). The combined organic layers were dried over anhydrous Na₂SO₄, and concentrated under reduced pressure. The residue was purified by prep-TLC and eluted with dichloromethane/methanol (12/1). The product was further purified by Prep-HPLC with XBridge Shield RP18 OBD column using water containing 0.1% NH₄HCO₃ and acetonitrile as mobile phase. Fractions containing the desired compound were evaporated to dryness to give 5.5 mg of 6-fluoro-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (isomer 1, EXAMPLE A117A, 8%) and 8.1 mg of 6-fluoro-15,21-dimethyl-5-(morpholin-4-yl)-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (isomer 1, EXAMPLE A118A, 11%) as a light yellow solid.

EXAMPLE 117A: LCMS: m/z (ESI), [M+H]⁺=518.25. ¹H NMR (DMSO-d₆, 400 MHz) δ 1.52-1.83 (3H, m), 2.02 (1H, d), 2.38-2.43 (2H, m), 2.51-2.65 (2H, m), 2.72-2.78 (1H, m), 3.21-3.27 (2H, m), 3.37-3.46 (1H, m), 3.63 (3H, s), 3.64-3.70 (1H, m), 3.72 (3H, s), 4.57 (1H, t), 4.71 (1H, d), 6.98-7.03 (1H, m), 7.37 (1H, dd), 7.72 (1H, dd), 8.29 (1H, d), 8.41 (1H, d), 8.83 (1H, s), 12.82 (1H, s).

EXAMPLE 118A: LCMS: m/z (ESI), [M+H]⁺=603.20. ¹H NMR (DMSO-d₆, 400 MHz) δ 1.48-1.82 (3H, m), 2.02 (1H, d), 2.38-2.49 (2H, m), 2.51-2.56 (2H, m), 2.62-2.77 (2H, m), 2.85-3.07 (4H, m), 3.21-3.27 (1H, m), 3.37-3.46 (1H, m), 3.62 (3H, s), 3.63-3.70 (1H, m), 3.72 (3H, s), 3.77 (4H, t), 4.57 (1H, t), 4.71 (1H, d), 7.21-7.43 (2H, m), 8.28 (1H, d), 8.41 (1H, d), 8.81 (1H, s), 12.71 (1H, s).

Example A117B, 6-fluoro-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (isomer 2) was prepared using same reaction sequence described in the synesthes is of EXAMPLE A117A from INT-A117B-6 as a light yellow solid. LCMS: m/z (EST), [M+H]⁺=518.30. ¹H NMR (DMSO-d₆, 400 MHz) δ 1.56-1.82 (3H, m), 2.02 (1H, d), 2.38-2.45 (2H, m), 2.51-2.55 (1H, m), 2.62-2.78 (2H, m), 3.15-3.25 (2H, m), 3.37-3.46 (1H, m), 3.63 (3H, s), 3.64-3.70 (1H, m), 3.72 (3H, s), 4.53 (1H, t), 4.65 (1H, d), 6.98-7.04 (1H, m), 7.37 (1H, dd), 7.72 (1H, dd), 8.29 (1H, d), 8.41 (1H, d), 8.83 (1H, s), 12.82 (1H, s).

Example A118B, 6-fluoro-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (isomer 2) was prepared using same reaction sequence described in the synesthesis of EXAMPLE A118A from INT-A117B-6 as a light yellow solid. LCMS: m/z (ESI), [M+H]⁺=603.30. ¹H NMR (DMSO-d₆, 400 MHz) δ 1.52-1.82 (3H, m), 2.03 (1H, d), 2.38-2.45 (2H, m), 2.51-2.62 (3H, m), 2.65-3.12 (6H, m), 3.21-3.27 (1H, m), 3.37-3.46 (1H, m), 3.62 (3H, s), 3.63-3.70 (1H, m), 3.72 (3H, s), 3.77 (4H, t), 4.55 (1H, t), 4.67 (1H, d), 7.29 (1H, d), 7.37 (1H, d), 8.27 (1H, s), 8.41 (1H, s), 8.81 (1H, s), 12.71 (1H, s).

### Example A119

### 5-[4-(dimethylamino)piperidin-1-yl]-15,21-dimethyl-23-oxa-2,9,11,15,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,17(34),18(22),19-octaene-12,16-dione

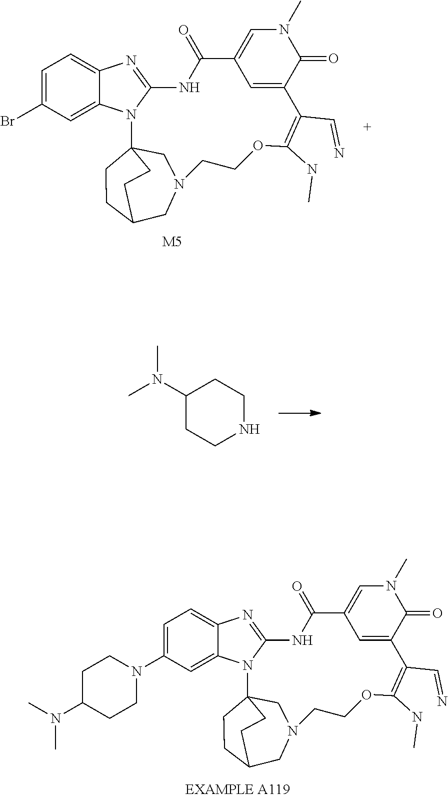

To a stirred mixture of 5-bromo-15,21-dimethyl-23-oxa-2,9,11,15,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,17(34),18(22),19-octaene-12,16-dione (M5, 100 mg, 0.16 mmol, 1 equiv), N,N-dimethylpiperidin-4-amine (64.92 mg, 0.50 mmol, 3 equiv) and BrettPhos Pd G₃ (45.90 mg, 0.05 mmol, 0.3 equiv) in 1,4-dioxane (10 mL) was added LiHMDS (1 mL, 1.014 mmol, 6 equiv) dropwise at room temperature under nitrogen atmosphere. The mixture was stirred for 1 h at 60 °C and then cooled to room temperature. The reaction was quenched with saturated NH₄Cl (aq.) (15 mL) and the mixture was extracted with dichloromethane (3×20 mL). The combined organic layers were washed with brine (3×30 mL), dried over anhydrous Na₂SO₄, and concentrated under reduced pressure. The residue was purified by prep-HPLC with XBridge Shield RP18 OBD column using water containing 10 mmol/L NH₄HCO₃ and 0.1% NH₃·H₂O and acetonitrile as mobile phase. Fractions containing the desired compound were evaporated to dryness to give 50.1 mg of 5-[4-(dimethylamino)piperidin-1-yl]-15,21-dimethyl-23-oxa-2,9,11,15,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,17(34),18(22),19-octaene-12,16-dione (EXAMPLE A119) as a light yellow solid (46%). LCMS: m/z (ESI), [M+H]⁺=640.40. ¹H NMR (DMSO-d₆, 400 MHz) δ 1.47-1.59 (2H, m), 1.81-1.99 (6H, m), 2.00-2.13 (3H, m), 2.22 (6H, s), 2.57-2.70 (2H, m), 2.75-2.82 (2H, m), 2.86 (2H, t), 3.38-3.50 (4H, m), 3.50-3.59 (3H, m), 3.62 (3H, s), 3.74 (3H, s), 4.27 (2H, t), 6.91 (1H, d), 7.34 (1H, s), 7.45 (1H, d), 8.30 (2H, d), 9.11 (1H, d), 13.18 (1H, s).

Example A120-EXAMPLE A128 (Table 2) were prepared using the same reaction sequence described above for the synthesis of EXAMPLE A119 from M5.

TABLE 2

| Example | | [M + H]⁺ | |
|---|---|---|---|
| No. | Structure | (observed) | ¹H NMR δ (ppm) |
| A120 | 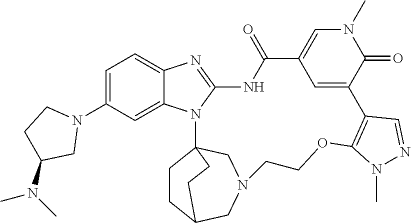 [SMILES: CN(C)[C@H]1CCN(c2ccc3nc4n(c3c2)C23CCC(CC2)CN(CCOc2c(cnn2C)-c2cc(cn(C)c2=O)C(=O)N4)C3)C1] | 626.40 | ¹H NMR (DMSO-d₆, 400 MHz) δ 1.77-2.00 (6H, m), 2.02-2.19 (5H, m), 2.21 (6H, s), 2.76-2.82 (2H, m), 2.87 (2H, t), 3.06 (1H, t), 3.21-3.37 (2H, m), 3.39-3.50 (4H, m), 3.61 (3H, s), 3.74 (3H, s), 4.27 (2H, t), 6.51 (1H, dd), 6.89 (1H, s), 7.42 (1H, d), 8.29 (2H, d), 9.11 (1H, d), 13.13 (1H, s). |
| A121 | 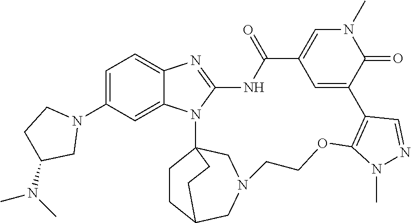 [SMILES: CN(C)[C@@H]1CCN(c2ccc3nc4n(c3c2)C23CCC(CC2)CN(CCOc2c(cnn2C)-c2cc(cn(C)c2=O)C(=O)N4)C3)C1] | 626.35 | ¹H NMR (DMSO-d₆, 400 MHz) δ 1.85-2.00 (6H, m), 2.02-2.17 (5H, m), 2.21 (6H, s), 2.77-2.83 (2H, m), 2.86 (2H, t), 3.06 (1H, t), 3.21-3.37 (2H, m), 3.40-3.49 (4H, m), 3.61 (3H, s), 3.74 (3H, s), 4.28 (2H, t), 6.52 (1H, d), 6.89 (1H, s), 7.43 (1H, d), 8.29 (2H, d), 9.11 (1H, d), 13.14 (1H, s) |
| A122 | 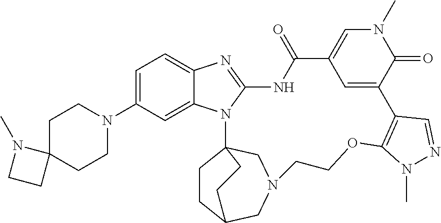 [SMILES: CN1CCC12CCN(c1ccc3nc4n(c3c1)C13CCC(CC1)CN(CCOc1c(cnn1C)-c1cc(cn(C)c1=O)C(=O)N4)C3)CC2] | 625.30 | ¹H NMR (DMSO-d₆, 400 MHz) δ 1.65-1.78 (2H, m), 1.78-1.84 (2H, m), 1.86-1.99 (6H, m), 2.01-2.12 (3H, m), 2.14 (3H, s), 2.57-2.71 (2H, m), 2.80 (2H, d), 2.86 (2H, t), 3.13 (2H, t), 3.38-3.51 (6H, m), 3.62 (3H, s), 3.74 (3H, s), 4.27 (2H, t), 6.91 (1H, dd), 7.33 (1H, d), 7.45 (1H, d), 8.29 (2H, d), 9.10 (1H, d), 13.18 (1H, s) |
| A123 | 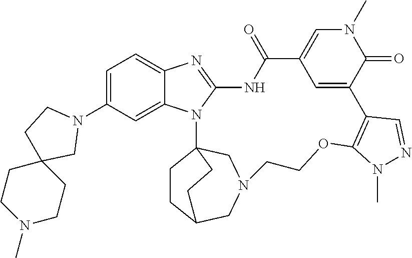 [SMILES: CN1CCC2(CC1)CCN(c1ccc3nc4n(c3c1)C13CCC(CC1)CN(CCOc1c(cnn1C)-c1cc(cn(C)c1=O)C(=O)N4)C3)C2] | 666.45 | ¹H NMR (DMSO-d₆, 400 MHz) δ 1.48-1.64 (4H, m), 1.83 (2H, t), 1.86- 1.98 (4H, m), 2.01-2.13 (3H, m), 2.18 (3H, s), 2.26-2.37 (4H, m), 2.80 (2H, d), 2.87 (2H, t), 3.08 (2H, s), 3.23- 3.31 (2H, m), 3.39-3.49 (4H, m), 3.61 (3H, s), 3.74 (3H, s), 4.28 (2H, t), 6.48 (1H, dd), 6.87 (1H, s), 7.41 (1H, d), 8.29 (2H, d), 9.11 (1H, d), 13.13 (1H, s). |
| A124 | 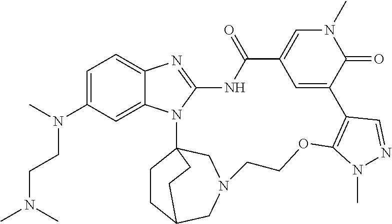 [SMILES: CN(C)CCN(C)c1ccc2nc3n(c2c1)C12CCC(CC1)CN(CCOc1c(cnn1C)-c1cc(cn(C)c1=O)C(=O)N3)C2] | 614.30 | ¹H NMR (DMSO-d₆, 400 MHz) δ 81.78-1.96 (4H, m), 2.00-2.16 (3H, m), 2.20 (6H, s), 2.39 (2H, t), 2.80 (2H, d), 2.87 (2H, t), 2.91 (3H, s), 3.34-3.51 (6H, m), 3.62 (3H, s), 3.74 (3H, s), 4.28 (2H, t), 6.68 (1H, dd), 7.11 (1H, s), 7.42 (1H, d), 8.29 (2H, d), 9.10 (1H, d), 13.13 (1H, s) |
| A125 | 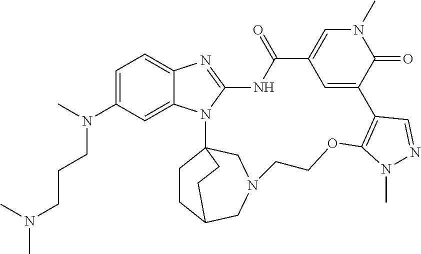 [SMILES: CN(C)CCCN(C)c1ccc2nc3n(c2c1)C12CCC(CC1)CN(CCOc1c(cnn1C)-c1cc(cn(C)c1=O)C(=O)N3)C2] | 628.35 | ¹H NMR (DMSO-d₆, 400 MHz) δ 1.58-1.72 (2H, m), 1.83-2.00 (4H, m), 2.07 (3H, dd), 2.14 (6H, s), 2.23 (2H, t), 2.80 (2H, d), 2.83-2.92 (5H, m), 3.31-3.35 (2H, m), 3.37-3.51 (4H, m), 3.61 (3H, s), 3.74 (3H, s), 4.27 (2H, t), 6.69 (1H, dd), 7.13 (1H, d), 7.41 (1H, d), 8.29 (2H, d), 9.11 (1H, d), 13.13 (1H, s) |
| A126 | 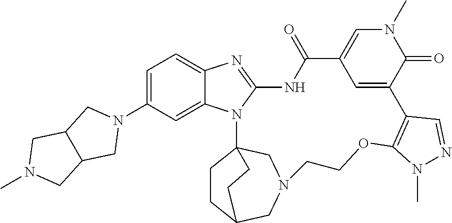 [SMILES: CN1CC2CN(c3ccc4nc5n(c4c3)C34CCC(CC3)CN(CCOc3c(cnn3C)-c3cc(cn(C)c3=O)C(=O)N5)C4)CC2C1] | 638.45 | ¹H NMR (DMSO-d₆, 400 MHz) δ 1.82-2.02 (4H, m), 2.02-2.15 (3H, m), 2.22 (3H, s), 2.35-2.44 (2H, m), 2.55- 2.62 (2H, m), 2.74-2.92 (6H, m), 3.08 (2H, dd), 3.39-3.48 (6H, m), 3.61 (3H, s), 3.74 (3H, s), 4.27 (2H, t), 6.62 (1H, d), 7.02 (1H, d), 7.42 (1H, d), 8.29 (2H, d), 9.11 (1H, d), 13.15 (1H, s) |
| A127 | 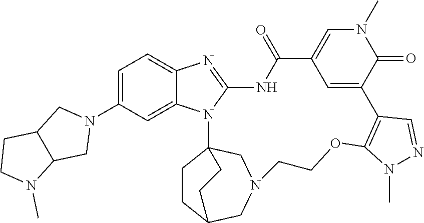 [SMILES: CN1CCC2CN(c3ccc4nc5n(c4c3)C34CCC(CC3)CN(CCOc3c(cnn3C)-c3cc(cn(C)c3=O)C(=O)N5)C4)CC21] | 638.40 | ¹H NMR (DMSO-d₆, 400 MHz) δ 1.58-1.68 (1H, m), 1.82-1.98 (4H, m), 2.00-2.14 (5H, m), 2.21-2.34 (1H, m), 2.30 (3H, s), 2.74-2.82 (2H, m), 2.83- 2.92 (2H, m), 2.98 (1H, t), 3.08-3.18 (2H, m), 3.22-3.32 (2H, m), 3.39-3.51 (5H, m), 3.62 (3H, s), 3.74 (3H, s), 4.28 (2H, t), 6.57 (1H, d), 6.96 (1H, s), 7.42 (1H, d), 8.29 (2H, d), 9.11 (1H, d), 13.15 (1H, s). |
| A128 | 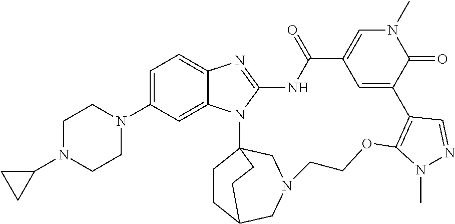 [SMILES: Cn1ncc2c1OCCN1CC3CCC(CC3)(C1)n1c(nc3ccc(N4CCN(C5CC5)CC4)cc31)NC(=O)c1cc-2c(=O)n(C)c1] | 638.35 | ¹H NMR (DMSO-d₆, 400 MHz) δ 0.34-0.35 (2H, m), 0.45-0.48 (2H, m), 1.60-1.69 (1H, m), 1.91-2.08 (8H, m), 2.70 (4H, t), 2.77-2.83 (2H, m), 2.86 (2H, t), 3.04 (4H, t), 3.41-3.52 (3H, m), 3.62 (3H, s), 3.74 (3H, s),4.27 (2H, d), 6.88-6.94 (1H, m), 7.32 (1H, s), 7.46 (1H, d), 8.30 (2H, d), 9.11 (1H, d), 13.18 (1H, s). |

### 21-methyl-5-(morpholin-4-yl)-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, Isomer 2 (Example A129B)

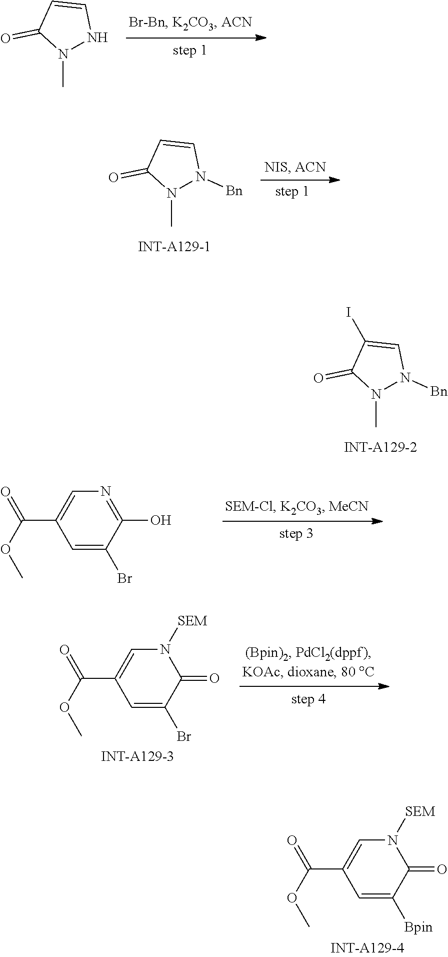

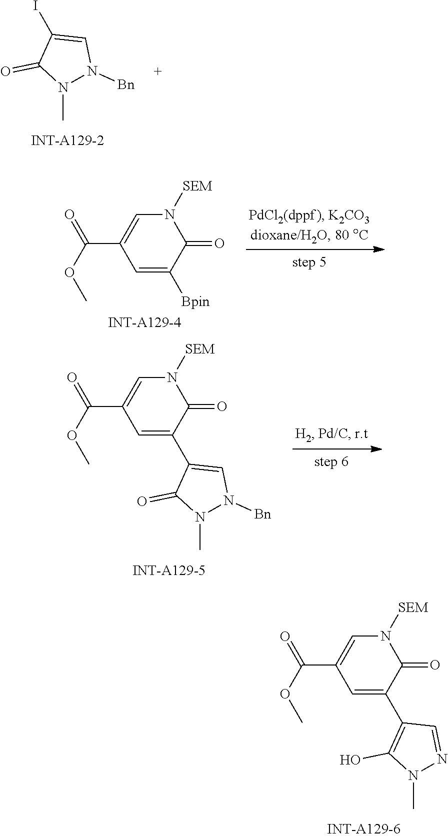

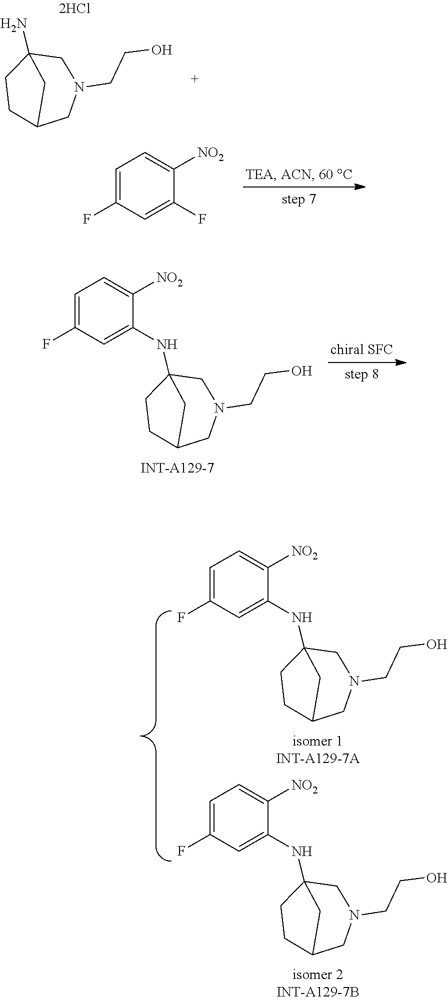 [SMILES: NC12CCC(CN(CCO)C1)C2 ; O=[N+]([O-])c1ccc(F)cc1F ; O=[N+]([O-])c1ccc(F)cc1NC12CCC(CN(CCO)C1)C2 ; O=[N+]([O-])c1ccc(F)cc1NC12CCC(CN(CCO)C1)C2 ; O=[N+]([O-])c1ccc(F)cc1NC12CCC(CN(CCO)C1)C2]

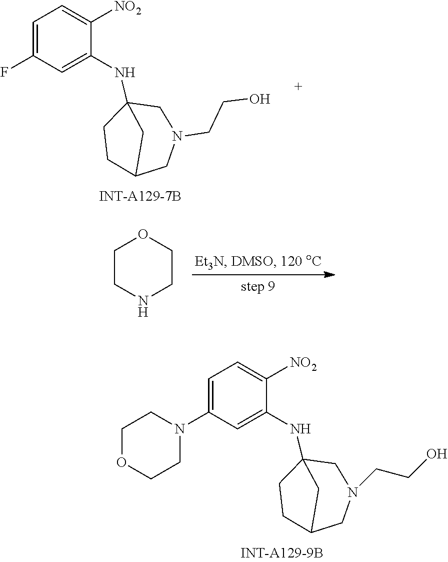 [SMILES: O=[N+]([O-])c1ccc(F)cc1NC12CCC(CN(CCO)C1)C2 ; C1COCCN1 ; O=[N+]([O-])c1ccc(N2CCOCC2)cc1NC12CCC(CN(CCO)C1)C2]

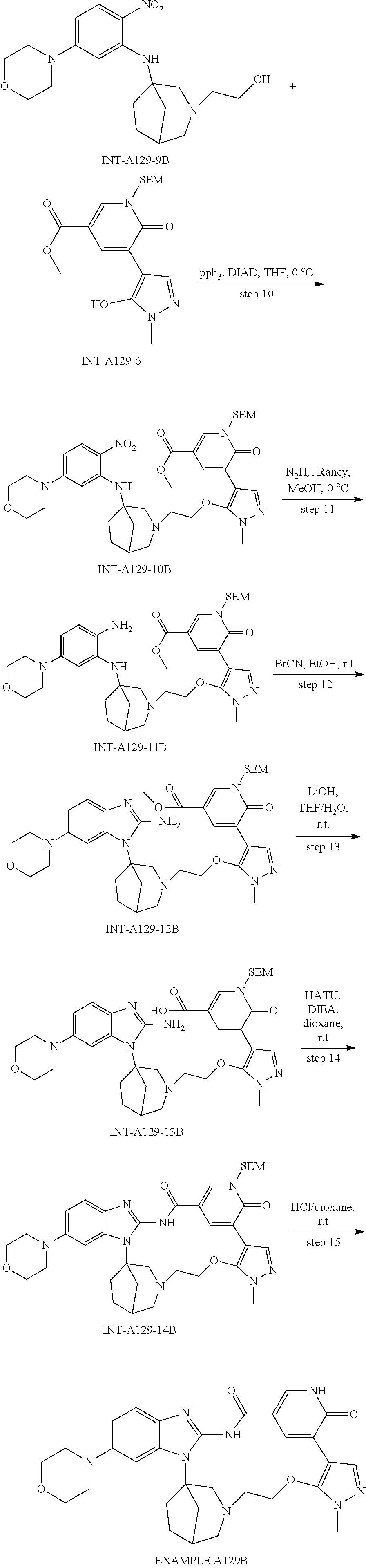

Step 1. Preparation of 1-benzyl-2-methylpyrazol-3-one (INT-A129-1). A mixture of 2-methyl-1H-pyrazol-3-one (10 g, 101.93 mmol, 1 equiv), K₂CO₃ (42.26 g, 305.79 mmol, 3 equiv), and BnBr (18.31 g, 107.02 mmol, 1.05 equiv) in acetonitrile (100 mL) was stirred for 2 h at room temperature under nitrogen atmosphere. The reaction was quenched with water (300 mL) and the mixture was extracted with ethyl acetate (3×200 mL). The combined organic layers were washed with brine (3×100 mL), dried over anhydrous Na₂SO₄, and concentrated under reduced pressure. The residue was purified by C18 FLASH using water and acetonitrile as mobile phase. Fractions containing the desired compound were evaporated to dryness to give 5.5 g of 1-benzyl-2-methylpyrazol-3-one (INT-A129-1) as a brown solid (28%). LCMS: m/z (ESI), [M+H]⁺=189.10. ¹H NMR (DMSO-d₆, 400 MHz) δ 3.14 (3H, s), 4.97 (2H, s), 5.23 (1H, d), 7.14-7.22 (2H, m), 7.26-7.38 (3H, m), 7.92 (1H, d).

Step 2. Preparation of 1-benzyl-4-iodo-2-methylpyrazol-3-one (INT-A129-2). To a stirred mixture of 1-benzyl-2-methylpyrazol-3-one (6.35 g, 33.73 mmol, 1 equiv) in acetonitrile (200 mL) was added NIS (8.35 g, 37.10 mmol, 1.1 equiv) in portions and stirred for 2 h at 0 °C under nitrogen atmosphere. The reaction was quenched with saturated sodium thiosufate solution (200 mL) and the mixture was extracted with ethyl acetate (3×300 mL). The combined organic layers were washed with brine (3×300 mL), dried over anhydrous Na₂SO₄, and concentrated under reduced pressure. The residue was purified by C18 FLASH using water and acetonitrile as mobile phase. Fractions containing the desired compound were evaporated to dryness to give 9.5 g of 1-benzyl-4-iodo-2-methylpyrazol-3-one (INT-A 129-2) as a reddish-brown semi-solid (89%). LCMS: m/z (ESI), [M+H]⁺=315.05. ¹H NMR (DMSO-d₆, 400 MHz) δ 3.22 (3H, s), 5.00 (2H, s), 7.16-7.41 (5H, m), 8.18 (1H, s).

Step 3. Preparation methyl 5-bromo-6-oxo-1-{[2-(trimethylsilyl)ethoxy]methyl}pyridine-3-carboxylate (INT-A129-3). To a stirred mixture of methyl 5-bromo-6-hydroxypyridine-3-carboxylate (10 g, 43.09 mmol, 1 equiv) and K₂CO₃ (11.91 g, 86.194 mmol, 2 equiv) in acetonitrile (200 mL) was added 2-(trimethylsily)ethoxymethyl chloride (10.78 g, 64.64 mmol, 1.5 equiv) dropwise at 0 °C under nitrogen atmosphere. The resulting mixture was stirred for 3 h at room temperature. The reaction was quenched with water (200 mL) and the mixture was extracted with ethyl acetate (3×200 mL). The combined organic layers were washed with brine (3×200 mL), dried over anhydrous Na₂SO₄, and concentrated under reduced pressure. The residue was re-crystallized from petroleum ether/ethyl acetate (20/1) to give 8 g of methyl 5-bromo-6-oxo-1-{[2-(trimethylsilyl)ethoxy]methyl}pyridine-3-carboxylate (INT-A129-3) as a white solid (51%). LCMS: m/z (ESI), [M+H]⁺=364.10. ¹H NMR (DMSO-d₆, 400 MHz) δ 0.00 (9H, s), 0.89-0.94 (2H, m), 3.60-3.68 (2H, m), 3.84 (3H, s), 5.44 (2H, s), 8.24 (1H, d), 8.58 (1H, d).

Step 4. Preparation of methyl 6-oxo-5-(4,4,5,5-tetramethyl-1,3,2-dioxaborolan-2-yl)-1-((2-(trimethylsilyl)ethoxy)methyl)-1,6-dihydropyridine-3-carboxylate (INT-A129-4). A mixture of methyl 5-bromo-6-oxo-1-{[2-(trimethylsilyl)ethoxy]methyl}pyridine-3-carboxylate (6.89 g, 19.01 mmol, 1 equiv), KOAc (5.60 g, 57.054 mmol, 3 equiv), Pd(dppf)Cl₂CH₂Cl₂ (1.55 g, 1.90 mmol, 0.1 equiv), and bis(pinacolato)diboron (7.24 g, 28.52 mmol, 1.5 equiv) in 1,4-dioxane (100 mL) was stirred for 2 h at 80 °C under nitrogen atmosphere and then cooled to room temperature. This mixture was used in the next step directly without further workup. LCMS: m/z (ESI), [M+H]⁺=328.05.

Step 5. Preparation of methyl 5-(1-benzyl-2-methyl-3-oxopyrazol-4-yl)-6-oxo-1-{[2-(trimethylsilyl)ethoxy]methyl}pyridine-3-carboxylate (INT-A129-5). A mixture of freshly prepared methyl 6-oxo-5-(4,4,5,5-tetramethyl-1,3,2-dioxaborolan-2-yl)-1-((2-(trimethylsilyl)ethoxy)methyl)-1,6-dihydropyridine-3-carboxylate (INT-A129-4), K₂CO₃ (5.28 g, 38.20 mmol, 3 equiv), Pd(dppf)Cl₂CH₂Cl₂ (1.04 g, 1.27 mmol, 0.1 equiv), and 1-benzyl-4-iodo-2-methylpyrazol-3-one (4 g, 12.73 mmol, 1.00 equiv) in dioxane (100 mL) and water (25 mL) was stirred for 2 h at 80 °C under nitrogen atmosphere. The reaction mixture was cooled to room temperature, treated with water (100 mL), and extracted with dichloromethane (3×100 mL). The combined organic layers were washed with brine (3×300 mL), dried over anhydrous Na₂SO₄, and concentrated under reduced pressure. The residue was purified by reversed-phase C18 flash chromatography using acetonitrile and water (0% to 100% gradient) as mobile phase to give 2.5 g of methyl 5-(1-benzyl-2-methyl-3-oxopyrazol-4-yl)-6-oxo-1-{[2-(trimethylsilyl)ethoxy]methyl}pyridine-3-carboxylate (INT-A 129-5) as a brown solid (410%). LCMS: m/z (ESI), [M+H]⁺=470.30.

Step 6. Preparation of methyl 5-(5-hydroxy-1-methylpyrazol-4-yl)-6-oxo-1-{[2-(trimethylsilyl)ethoxy]methyl}pyridine-3-carboxylate (INT-A129-6). A mixture of methyl 5-(1-benzyl-2-methyl-3-oxopyrazol-4-yl)-6-oxo-1-{[2-(trimethylsilyl)ethoxy]methyl}pyridine-3-carboxylate (2.5 g, 5.32 mmol, 1.00 equiv) and Pd/C (2.83 g, 26.62 mmol, 5 equiv) in methanol (50 mL) was stirred overnight at room temperature under hydrogen atmosphere. The resulting mixture was filtered. The filter cake was washed with methanol (3×100 mL). The filtrate was concentrated under reduced pressure to give 1.5 g of methyl 5-(5-hydroxy-1-methylpyrazol-4-yl)-6-oxo-1-{[2-(trimethylsilyl)ethoxy]methyl}pyridine-3-carboxylate (INT-A129-6) as a brown solid (74%). LCMS: m/z (ESI), [M+H]⁺=380.00.

Step 7. Preparation of 2-{l-[(5-fluoro-2-nitrophenyl)amino]-3-azabicyclo[3.2.1]octan-3-yl}ethanol (INT-A129-7). A mixture of 2-{1-amino-3-azabicyclo[3.2.1]octan-3-yl}ethanol dihydrochloric (4.8 g, 19.75 mmol, 1 equiv), trimethylamine (9.99 g, 98.77 mmol, 5 equiv) and 2,4-difluoro-1-nitrobenzene (4.71 g, 29.63 mmol, 1.5 equiv) in acetonitrile (100 mL) was stirred for 2 h at 60 °C under nitrogen atmosphere and cooled to room temperature. The reaction was quenched with water (300 mL) and the mixture was extracted with ethyl acetate (3×200 mL). The combined organic layers were washed with brine (3×300 mL), dried over anhydrous Na₂SO₄, and concentrated under reduced pressure. The residue was purified on a silica gel column eluted with petroleum ether/ethyl acetate (2/1) to give 5.5 g of 2-{1-[(5-fluoro-2-nitrophenyl)amino]-3-azabicyclo[3.2.1]octan-3-yl}ethanol (INT-A129-7) as an orange oil (90%). LCMS: m/z (ESI), [M+H]⁺=310.10. ¹H NMR (DMSO-d₆, 400 MHz) δ 1.58-1.73 (3H, m), 1.76-1.98 (2H, m), 2.08-2.25 (4H, m), 2.36-2.51 (2H, m), 2.68 (1H, dd), 3.12-3.20 (1H, m), 3.44-3.58 (2H, m), 4.35 (1H, t), 6.53-6.62 (1H, m), 6.83 (1H, dd), 8.19 (1H, dd), 8.43-8.48 (1H, m).

Step 8. Preparation of 2-{1-[(5-fluoro-2-nitrophenyl)amino]-3-azabicyclo[3.2.1]octan-3-yl}ethanol (isomer 2, INT-A129-7B). The racemic mixture of 2-{1-[(5-fluoro-2-nitrophenyl)amino]-3-azabicyclo[3.2.1]octan-3-yl}ethanol (15 g) was separated by Prep_SFC (CHIRALPAK IG, 3*25 cm, 5 m), eluted with mobile phase A of CO₂ and mobile phase B of methanol, to give 6.2 g (41%) isomer 1 (INT-A129-7A) as an orange oil and 6.5 g of isomer 2 (INT-A129-7B) as a yellowish oil (43%).

Isomer 1: LCMS: m/z (ESI), [M+H]⁺=310.20. Rt=1.429 min.

Isomer 2: LCMS: m/z (ESI), [M+H]⁺=310.20. ¹H NMR (DMSO-d₆, 400 MHz) δ 1.59-1.73 (3H, m), 1.82 (1H, d), 1.94 (1H, s), 2.17 (2H, dd), 2.44 (2H, d), 2.68 (1H, d), 3.17 (2H, d), 3.51 (2H, s), 4.11 (1H, q), 4.35 (1H, d), 6.58 (1H, t), 6.83 (1H, d), 8.19 (1H, dd), 8.45 (1H, s). Rt=1.987 min.

Step 9. Preparation of 2-(1-{[5-(morpholin-4-yl)-2-nitrophenyl]amino}-3-azabicyclo[3.2.1]octan-3-yl)ethanol (INT-A129-9B). A mixture of 2-{1-[(5-fluoro-2-nitrophenyl)amino]-3-azabicyclo[3.2.1]octan-3-yl}ethanol (INT-A129-7B, 6.5 g, 21.01 mmol, 1 equiv), morpholine (6.4 g, 73.54 mmol, 3.5 equiv) and trimethylamine (6.38 g, 63.03 mmol, 3 equiv) in dimethyl sulfoxide (100 mL) was stirred for 2 h at 120 °C under nitrogen atmosphere. The mixture was cooled to room temperature. The reaction was quenched with water (800 mL) and the mixture was extracted with ethyl acetate (3×500 mL). The combined organic layers were washed with brine (3×500 mL), dried over anhydrous Na₂SO₄, and concentrated under reduced pressure to give 7.4 g of 2-(1-{[5-(morpholin-4-yl)-2-nitrophenyl]amino}-3-azabicyclo[3.2.1]octan-3-yl)ethanol (INT-A129-9B) as an orange solid (93%). LCMS: m/z (ESI), [M+H]⁺=377.25. ¹H NMR (DMSO-d₆, 400 MHz) δ 1.63-1.71 (3H, m), 1.83-1.87 (1H, m), 1.92-1.96 (1H, m), 2.05 (1H, d), 2.14-2.22 (2H, m), 2.32-2.51 (3H, m), 2.51-2.57 (1H, m), 2.65 (1H, dd), 3.31-3.37 (4H, m), 3.45-3.56 (2H, m), 3.73 (4H, t), 4.35 (1H, t), 6.09 (1H, d), 6.46 (1H, dd), 7.93 (1H, d), 8.67 (1H, s).

Step 10. Preparation of methyl 5-(1-methyl-5-{2-[1-{[5-(morpholin-4-yl)-2-nitrophenyl]amino}-3-azabicyclo[3.2.1]octan-3-yl]ethoxy}pyrazol-4-yl)-6-oxo-1-{[2-(trimethylsilyl)ethoxy]methyl}pyridine-3-carboxylate (INT-A129-10B). To a stirred mixture of methyl 5-(5-hydroxy-1-methylpyrazol-4-yl)-6-oxo-1-{[2-(trimethylsilyl)ethoxy]methyl}pyridine-3-carboxylate (INT-A129-6, 756 mg, 1.99 mmol, 1.5 equiv), 2-[1-{[5-(morpholin-4-yl)-2-nitrophenyl]amino}-3-azabicyclo[3.2.1]octan-3-yl]ethanol (INT-A129-9B, 500 mg, 1.32 mmol, 1.00 equiv) and triphenylphosphine (1045 mg, 3.98 mmol, 3 equiv) in tetrahydrofuran (10 mL) was added diisopropyl azodicarboxylate (805 mg, 3.98 mmol, 3 equiv) dropwise at 0 °C under nitrogen atmosphere. The resulting mixture was stirred for 2 h at 0 °C under nitrogen atmosphere. The reaction was quenched with water (100 mL) and the mixture was extracted with ethyl acetate (3×100 mL). The combined organic layers were washed with brine (3×100 mL), dried over anhydrous Na₂SO₄, and concentrated under reduced pressure. The residue was purified on a Prep-TLC eluted with petroleum ether/ethyl acetate (1/3) to give 400 mg of methyl 5-(1-methyl-5-{2-[1-{[5-(morpholin-4-yl)-2-nitrophenyl]amino}-3-azabicyclo[3.2.1]octan-3-yl]ethoxy}pyrazol-4-yl)-6-oxo-1-{[2-(trimethylsilyl)ethoxy]methyl}pyridine-3-carboxylate (INT-A129-10B) as an orange solid (40%). LCMS: m/z (ESI), [M+H]⁺=738.60.

Step 11. Preparation of methyl 5-(5-{2-[1-{[2-amino-5-(morpholin-4-yl)phenyl]amino}-3-azabicyclo[3.2.1]octan-3-yl]ethoxy}-1-methylpyrazol-4-yl)-6-oxo-1-{[2-(trimethylsilyl)ethoxy]methyl}pyridine-3-carboxylate (INT-A129-111B). To a stirred mixture of methyl 5-(1-methyl-5-{2-[1-{[5-(morpholin-4-yl)-2-nitrophenyl]amino}-3-azabicyclo[3.2.1]octan-3-yl]ethoxy}pyrazol-4-yl)-6-oxo-1-{[2-(trimethylsilyl)ethoxy]methyl}pyridine-3-carboxylate (INT-A129-10B, 380 mg, 0.51 mmol, 1 equiv) and Raney Ni (220.59 mg, 2.57 mmol, 5 equiv) in methanol (10 mL) was added hydrazine hydrate (98%) (38.67 mg, 0.77 mmol, 1.5 equiv) dropwise and stirred for 2 h at 0 °C under nitrogen atmosphere. The resulting mixture was filtered. The filter cake was washed with methanol (3×50 mL). The filtrate was concentrated under reduced pressure. The crude product was used in the next step directly without further purification. LCMS: m/z (ESI), [M+H]⁺=708.70.

Step 12. Preparation of methyl 5-(5-{2-[1-[2-amino-6-(morpholin-4-yl)-1,3-benzodiazol-1-yl]-3-azabicyclo[3.2.1]octan-3-yl]ethoxy}-1-methylpyrazol-4-yl)-6-oxo-1-{[2-(trimethylsilyl)ethoxy]methyl}pyridine-3-carboxylate (INT-A129-12B). A mixture of methyl 5-(5-{2-[1-{[2-amino-5-(morpholin-4-yl)phenyl]amino}-3-azabicyclo[3.2.1]octan-3-yl]ethoxy}-1-methylpyrazol-4-yl)-6-oxo-1-{[2-(trimethylsilyl)ethoxy]methyl}pyridine-3-carboxylate (INT-A129-11B) and BrCN (62.54 mg, 0.59 mmol, 1.1 equiv) in ethanol (10 mL) was stirred overnight at room temperature under nitrogen atmosphere. The reaction was quenched with saturated NaHCO₃ solution (50 mL) and the mixture was extracted with ethyl acetate (3×100 mL). The combined organic layers were washed with brine (3×100 mL), dried over anhydrous Na₂SO₄, and concentrated under reduced pressure. The residue was purified by Prep-TLC eluted with dichloromethane/methanol (15/1) to give 250 mg of methyl 5-(5-{2-[1-[2-amino-6-(morpholin-4-yl)-1,3-benzodiazol-1-yl]-3-azabicyclo[3.2.1]octan-3-yl]ethoxy}-1-methylpyrazol-4-yl)-6-oxo-1-{[2-(trimethylsilyl)ethoxy]methyl}pyridine-3-carboxylate (INT-A129-12B) as a brown solid (63%). LCMS: m/z (ESI), [M+H]⁺=733.65.

Step 13. Preparation of 5-(5-{2-[1-[2-amino-6-(morpholin-4-yl)-1,3-benzodiazol-1-yl]-3-azabicyclo[3.2.1]octan-3-yl]ethoxy}-1-methylpyrazol-4-yl)-6-oxo-1-{[2-(trimethylsilyl)ethoxy]methyl}pyridine-3-carboxylic acid (INT-A129-13B). A mixture of methyl 5-(5-{2-[1-[2-amino-6-(morpholin-4-yl)-1,3-benzodiazol-1-yl]-3-azabicyclo[3.2.1]octan-3-yl]ethoxy}-1-methylpyrazol-4-yl)-6-oxo-1-{[2-(trimethylsilyl)ethoxy]methyl}pyridine-3-carboxylate (INT-A129-12B, 80 mg, 0.10 mmol, 1 equiv) and LiOH (4 mg, 0.17 mmol, 1.5 equiv) in tetrahydrofuran (2 mL) and water (0.5 mL) was stirred overnight at room temperature under nitrogen atmosphere. The resulting mixture was concentrated under reduced pressure. The resulting mixture was used in the next step directly without further purification. LCMS: m/z (ESI), [M+H]⁺=719.55.

Step 14. Preparation of 21-methyl-5-(morpholin-4-yl)-15-{[2-(trimethylsilyl)ethoxy]methyl}-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (INT-A129-14B). A mixture of 5-(5-{2-[1-[2-amino-6-(morpholin-4-yl)-1,3-benzodiazol-1-yl]-3-azabicyclo[3.2.1]octan-3-yl]ethoxy}-1-methylpyrazol-4-yl)-6-oxo-1-{[2-(trimethylsilyl)ethoxy]methyl}pyridine-3-carboxylic acid (INT-A129-13B, 80 mg, 0.10 mmol, 1 equiv), N,N,N,N-tetramethyl-O-(7-azabenzotriazol-1-yl)uronium hexafluorophospate (63 mg, 0.16 mmol, 1.5 equiv) and N,N-diisopropylethylamine (43 mg, 0.33 mmol, 3 equiv) in dioxane (5 mL) was stirred for 2 h at room temperature under nitrogen atmosphere. The reaction was quenched with water (50 mL) and the mixture was extracted with ethyl acetate (3×50 mL). The combined organic layers were washed with brine (3×50 mL), dried over anhydrous Na₂SO₄, and concentrated under reduced pressure. The residue was purified by Prep-TLC eluted with dichloromethane/methanol (20/1) to give 50 mg of 21-methyl-5-(morpholin-4-yl)-15-{[2-(trimethylsilyl)ethoxy]methyl}-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (INT-A129-14B) as a white solid (64%). LCMS: m/z (ESI), [M+H]⁺=701.60.

Step 15. Preparation of 21-methyl-5-(morpholin-4-yl)-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (EXAMPLE A129B). A mixture of 21-methyl-5-(morpholin-4-yl)-15-{[2-(trimethylsilyl)ethoxy]methyl}-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (INT-A129-14B, 45 mg, 0.06 mmol, 1 equiv) in HCl solution of 1,4-dioxane (2 mL) was stirred for 2 h at room temperature under nitrogen atmosphere. The mixture was neutralized to pH 7 with saturated NaHCO₃ solution. The mixture was extracted with dichloromethane (3×20 mL) and the combined organic layers were concentrated under reduced pressure. The residue was purified by Prep-HPLC (XBridge Prep OBD C18 Column, 30*150 mm, 5 μm) using mobile phase A of water (containing 10 mmol/L NH₄HCO₃ and 0.05% NH₃·H₂O) and mobile phase B of CAN to give 14.3 mg of 21-methyl-5-(morpholin-4-yl)-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione as a white solid (39%). LCMS: m/z (ESI), [M+H]⁺=571.45. ¹H NMR (DMSO-d₆, 400 MHz) δ 1.50-1.83 (4H, m), 2.00 (1H, d), 2.71 (2H, s), 3.05-3.15 (5H, m), 3.17-3.23 (3H, m), 3.62-3.92 (9H, m), 4.56 (1H, t), 4.65-4.72 (1H, m), 6.90 (1H, d), 7.19 (1H, s), 7.41 (1H, d), 7.87 (1H, s), 8.40 (1H, s), 8.82 (1H, d), 11.96 (1H, s), 12.62 (1H, s).

### Example A130B

### 15-methyl-5-(morpholin-4-yl)-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18,21-octaene-12,16-dione, Isomer 2 (Example A130B)

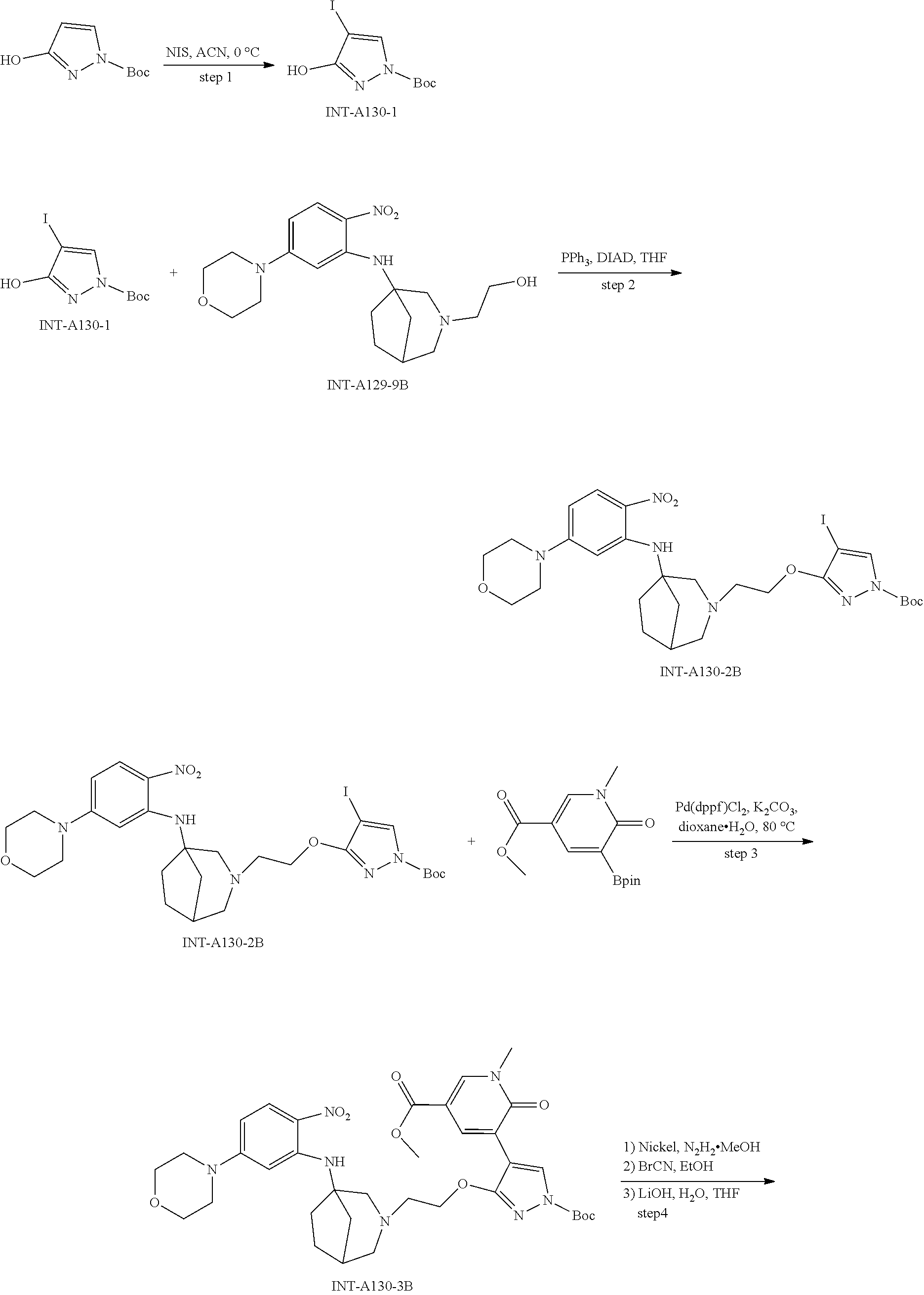

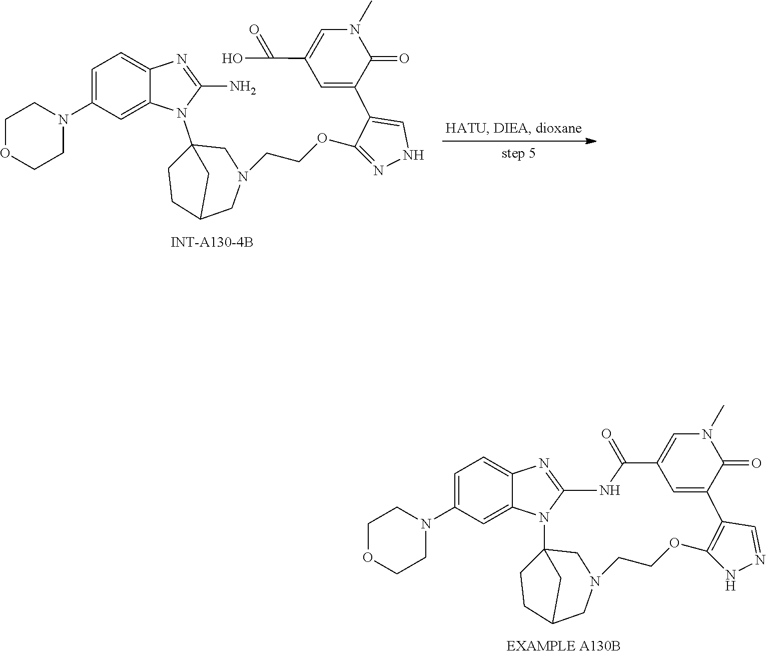 [SMILES: Cn1cc(C(=O)O)cc(-c2c[nH]nc2OCCN2CC3CCC(n4c(N)nc5ccc(N6CCOCC6)cc54)(C3)C2)c1=O ; Cn1cc2cc(c1=O)-c1cn[nH]c1OCCN1CC3CCC(C3)(C1)n1c(nc3ccc(N4CCOCC4)cc31)NC2=O]

Step 1. Preparation of tert-butyl 3-hydroxy-4-iodopyrazole-1-carboxylate (INT-A130-1). To a stirred mixture of tert-butyl 3-hydroxypyrazole-1-carboxylate (4 g, 21.71 mmol, 1 equiv) in acetonitrile (80 mL) was added NIS (5.86 g, 26.05 mmol, 1.2 equiv) in portions at 0 °C under nitrogen atmosphere. The resulting mixture was stirred for 2 h at 0 °C under nitrogen atmosphere. The reaction was quenched with saturated sodium thiosufate solution and the mixture was extracted with ethyl acetate (3×200 mL). The combined organic layers were washed with brine (3×200 mL), dried over anhydrous Na₂SO₄, and concentrated under reduced pressure. The residue was purified by C18 flash using water and acetonitrile as mobile phase. Fractions containing the desired compound were evaporated to dryness to give 2.5 g tert-butyl 3-hydroxy-4-iodopyrazole-1-carboxylate as a brown solid (40%). LCMS: m/z (ESI), [M+H]⁺=311.00. ¹H NMR (DMSO-d₆, 400 MHz) δ 1.53 (9H, s), 8.18 (1H, s), 11.63 (1H, s).

Step 2. Preparation of tert-butyl 4-iodo-3-{2-[1-{[5-(morpholin-4-yl)-2-nitrophenyl]amino}-3-azabicyclo[3.2.1]octan-3-yl]ethoxy}pyrazole-1-carboxylate (INT-A130-2B). To a stirred mixture of 2-[1-{[5-(morpholin-4-yl)-2-nitrophenyl]amino}-3-azabicyclo[3.2.1]octan-3-yl]ethanol (INT-A129-9B, 500 mg, 1.32 mmol, 1.00 equiv) and triphenylphosphine (1045 mg, 3.98 mmol, 3 equiv) in tetrahydrofuran (10 mL) were added diisopropyl azodicarboxylate (805 mg, 3.98 mmol, 3 equiv) and tert-butyl 3-hydroxy-4-iodopyrazole-1-carboxylate (617 mg, 1.99 mmol, 1.5 equiv) at 0 °C under nitrogen atmosphere. The resulting mixture was stirred for 2 h at 0 °C under nitrogen atmosphere. The reaction was quenched with water (100 mL) and the mixture was extracted with dichloromethane (3×100 mL). The combined organic layers were washed with brine (3×100 mL), dried over anhydrous Na₂SO₄, and concentrated under reduced pressure. The residue was purified by Prep-TLC eluted with petroleum ether/ethyl acetate (1/1) to give 550 mg of tert-butyl 4-iodo-3-{2-[1-{[5-(morpholin-4-yl)-2-nitrophenyl]amino}-3-azabicyclo[3.2.1]octan-3-yl]ethoxy}pyrazole-1-carboxylate as a yellow solid (61%). LCMS: m/z (ESI), [M+H]⁺=669.40.

Step 3. Preparation methyl 5-[1-(tert-butoxycarbonyl)-3-{2-[1-{[5-(morpholin-4-yl)-2-nitrophenyl]amino}-3-azabicyclo[3.2.1]octan-3-yl]ethoxy}pyrazol-4-yl]-1-methyl-6-oxopyridine-3-carboxylate (INT-A130-3B). A mixture of tert-butyl 4-iodo-3-{2-[1-{[5-(morpholin-4-yl)-2-nitrophenyl]amino}-3-azabicyclo[3.2.1]octan-3-yl]ethoxy}pyrazole-1-carboxylate (INT-A130-3B, 550 mg, 0.823 mmol, 1 equiv), K₂CO₃ (341 mg, 2.46 mmol, 3 equiv), Pd(dppf)Cl₂CH₂Cl₂ (67 mg, 0.08 mmol, 0.1 equiv), and methyl 1-methyl-6-oxo-5-(4,4,5,5-tetramethyl-1,3,2-dioxaborolan-2-yl)pyridine-3-carboxylate (361 mg, 1.23 mmol, 1.5 equiv) in dioxane (20 mL) and water (5 mL) was stirred for 2 h at 80 °C under nitrogen atmosphere. The mixture was cooled to room temperature, treated with water (100 mL), and extracted with dichloromethane (3×100 mL). The combined organic layers were washed with brine (3×100 mL), dried over anhydrous Na₂SO₄, and concentrated under reduced pressure. The residue was purified by C18 FLASH using water containing 10 mmol/L NH₄HCO₃ and 0.1% NH₃—H₂O and acetonitrile as mobile phase. Fractions containing the desired compound were evaporated to dryness to give 130 mg of methyl 5-[1-(tert-butoxycarbonyl)-3-{2-[1-{[5-(morpholin-4-yl)-2-nitrophenyl]amino}-3-azabicyclo[3.2.1]octan-3-yl]ethoxy}pyrazol-4-yl]-1-methyl-6-oxopyridine-3-carboxylate as a yellow solid (22%). LCMS: m/z (ESI), [M+H]⁺=708.55.

Step 4. Preparation of 5-(3-{2-[1-[2-amino-6-(morpholin-4-yl)-1,3-benzodiazol-1-yl]-3-azabicyclo[3.2.1]octan-3-yl]ethoxy}-1H-pyrazol-4-yl)-1-methyl-6-oxopyridine-3-carboxylic acid (INT-A130-4B). To a stirred mixture of Raney-Ni (6.05 mg, 0.07 mmol, 1 equiv) and methyl 5-[1-(tert-butoxycarbonyl)-3-{2-[1-{[5-(morpholin-4-yl)-2-nitrophenyl]amino}-3-azabicyclo[3.2.1]octan-3-yl]ethoxy}pyrazol-4-yl]-1-methyl-6-oxopyridine-3-carboxylate (50 mg, 0.07 mmol, 1 equiv) in methanol (1 mL) was added hydrazine hydrate (10.61 mg, 0.21 mmol, 3 equiv) at 0 °C under nitrogen atmosphere. The resulting mixture was stirred for 2 h at room temperature. The resulting mixture was filtered, and the filter cake was washed with methanol (3×5 mL). The filtrate was concentrated under reduced pressure. To this residue was added ethanol (1 mL) and BrCN (8.23 mg, 0.07 mmol, 1.1 equiv). The resulting mixture was stirred for additional 2 h at room temperature and treated with solution of LiOH·H₂O (5.93 mg, 0.14 mmol, 2 equiv) in H₂O (0.2 mL). The resulting mixture was stirred for 2 h at 40 °C and concentrated under reduced pressure. The residue was purified by reverse flash chromatography, eluted with 10% to 50% acetonitrile in water, to give 15 mg of 5-(3-{2-[(1S,5S)-1-[2-amino-6-(morpholin-4-yl)-1,3-benzodiazol-1-yl]-3-azabicyclo[3.2.1]octan-3-yl]ethoxy}-1H-pyrazol-4-yl)-1-methyl-6-oxopyridine-3-carboxylic acid as a white solid (36%). LCMS: m/z (ESI), [M+H]⁺=589.40.

Step 5. Preparation of 15-methyl-5-(morpholin-4-yl)-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18,21-octaene-12,16-dione (EXAMPLE A130B). A mixture of 5-(3-{2-[1-[2-amino-6-(morpholin-4-yl)-1,3-benzodiazol-1-yl]-3-azabicyclo[3.2.1]octan-3-yl]ethoxy}-1H-pyrazol-4-yl)-1-methyl-6-oxopyridine-3-carboxylic acid (INT-A130-4B, 20 mg, 0.03 mmol, 1 equiv), N,N,N,N-Tetramethyl-O-(7-azabenzotriazol-1-yl)uronium hexafluorophospate (19.38 mg, 0.05 mmol, 1.5 equiv) and N,N-diisopropylethylamine (13.17 mg, 0.10 mmol, 3 equiv) in 1,4-dioxane (2 mL) was stirred for 2 h at 40 °C under nitrogen atmosphere. The reaction was quenched with water (30 mL) and the mixture was extracted with ethyl acetate (3×30 mL). The combined organic layers were washed with brine (3×30 mL), dried over anhydrous Na₂SO₄, and concentrated under reduced pressure. The residue was purified on a silica gel column eluted with dichloromethane/methanol (25/1) to give 4.9 mg of 15-methyl-5-(morpholin-4-yl)-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18,21-octaene-12,16-dione as a white solid (25%). LCMS: m/z (ESI), [M+H]⁺=571.20. ¹H NMR (DMSO-d₆, 400 MHz) δ 1.51-1.61 (2H, m), 1.68 (1H, d), 2.45-2.67 (6H, m), 3.09 (4H, q), 3.37-3.55 (3H, m), 3.62 (3H, s), 3.76 (4H, t), 4.14 (2H, d), 4.32 (1H, d), 6.87-6.93 (1H, m), 7.18 (1H, d), 7.42 (1H, d), 8.20 (1H, d), 8.44 (1H, d), 9.19 (1H, d), 11.95 (1H, s), 12.53 (1H, s).

### Example A132B

### 5-hydroxy-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, Isomer 2 (Example A132B)

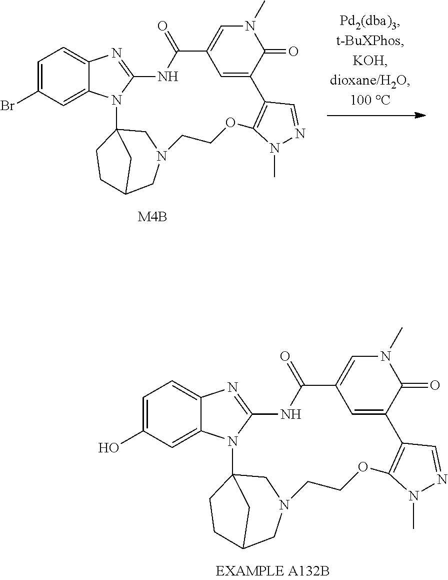 [SMILES: Cn1ncc2c1OCCN1CC3CCC(C3)(C1)n1c(nc3ccc(Br)cc31)NC(=O)c1cc-2c(=O)n(C)c1 ; Cn1ncc2c1OCCN1CC3CCC(C3)(C1)n1c(nc3ccc(O)cc31)NC(=O)c1cc-2c(=O)n(C)c1]

A mixture of 5-bromo-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (M4B, 150 mg, 0.26 mmol, 1 equiv), Pd₂(dba)₃ (47.49 mg, 0.05 mmol, 0.2 equiv), t-BuXPhos (22.02 mg, 0.052 mmol, 0.2 equiv), KOH (58.19 mg, 1.036 mmol, 4 equiv) in 1,4-dioxane (3 mL) and H₂O (2 mL) was stirred for 2 h at 100 °C under nitrogen atmosphere. The reaction was quenched with water (50 mL) and the mixture was extracted with ethyl acetate (3×50 mL). The combined organic layers were washed with brine (3×50 mL), dried over anhydrous Na₂SO₄, and concentrated under reduced pressure. The residue was purified by C18 flash using water and acetonitrile as mobile phase. Fractions containing the desired compound were evaporated to dryness to give 4.8 mg of 5-hydroxy-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (isomer 2) as a white solid (3%). LCMS: m/z (EST), [M+H]⁺=516.25. ¹H NMR (DMSO-d₆, 400 MHz) δ 1.52-1.63 (1H, m), 1.63-1.79 (2H, m), 1.99 (1H, d), 2.42-2.72 (8H, m), 3.62 (3H, s), 3.63-3.69 (1H, m), 3.71 (3H, s), 4.54 (1H, t), 4.67 (1H, d), 6.63 (1H, dd), 7.16 (1H, d), 7.35 (1H, d), 8.26 (1H, d), 8.41 (1H, s), 8.81 (1H, d), 9.32 (1H, s), 12.55 (1H, s).

### Example A133B

### 5-[(2-hydroxyethyl)amino]-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione, isomer 2

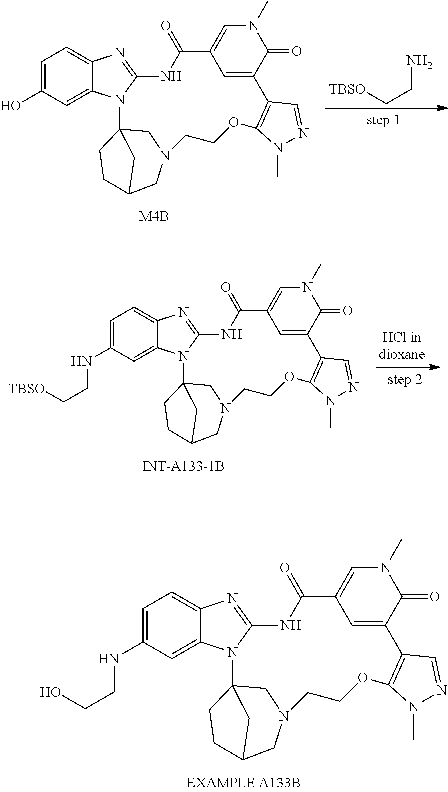 [SMILES: Cn1ncc2c1OCCN1CC3CCC(C3)(C1)n1c(nc3ccc(O)cc31)NC(=O)c1cc-2c(=O)n(C)c1 ; CC(C)(C)[Si](C)(C)OCCN ; Cn1ncc2c1OCCN1CC3CCC(C3)(C1)n1c(nc3ccc(NCCO[Si](C)(C)C(C)(C)C)cc31)NC(=O)c1cc-2c(=O)n(C)c1 ; Cn1ncc2c1OCCN1CC3CCC(C3)(C1)n1c(nc3ccc(NCCO)cc31)NC(=O)c1cc-2c(=O)n(C)c1]

Step 1. 5-({2-[(tert-butyldimethylsilyl)oxy]ethyl}amino)-15,21-dimethyl-23-oxa-2,9,11,15,20,21,26-heptaazaheptacyclo[24.4.1.1\hat{ }{1,28}.1 \hat{ }{13,17}.0\hat{ }{2,10}.0\hat{ }{3,8}.0\hat{ }{18,22}]tritriaconta-3,5,7,9,13,17(33),18(22),19-octaene-12,16-dione (INT-A133-1B). A mixture of 5-bromo-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (120 mg, 0.20 mmol, 1 equiv), (2-aminoethoxy)(tert-butyl)dimethylsilane (M4B, 109.12 mg, 0.62 mmol, 3 equiv), BrettPhos Pd G3 (56.41 mg, 0.06 mmol, 0.3 equiv) and LiHMDS (347.12 mg, 2.07 mmol, 10 equiv) in dioxane (5 mL) was stirred for 2 h at 60 °C under nitrogen atmosphere. The reaction was quenched with NH₄Cl (aq.) (50 mL) and the mixture was extracted with ethyl acetate (3×50 mL). The combined organic layers were washed with brine (3×50 mL), dried over anhydrous Na₂SO₄, and concentrated under reduced pressure. The residue was purified by silica gel column chromatography, eluted with dichloromethane/methanol (20:1), to give 65 mg of 5-({2-[(tert-butyldimethylsilyl)oxy]ethyl}amino)-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione as a yellow solid (46%). LCMS: m/z (ESI), [M+H]⁺=673.30.

Step 2. 5-[(2-hydroxyethyl)amino]-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione (EXAMPLE A133B). A solution of 5-({2-[(tert-butyldimethylsilyl)oxy]ethyl}amino)-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}1]tritriaconta-3,5,7,9,13,17(33),18(22),19-octaene-12,16-dione (INT-A133-1B, 60 mg, 0.09 mmol, 1 equiv) in a solution of HCl in 1,4-dioxane (2 mL) was stirred for 2 h at 15 °C under nitrogen atmosphere. The reaction was quenched with NaHCO₃ solution (50 mL) and the mixture was extracted with ethyl acetate (3×50 mL). The combined organic layers were washed with brine (3×50 mL), dried over anhydrous Na₂SO₄, and concentrated under reduced pressure. The residue was purified by C18 flash using water and acetonitrile as mobile phase. Fractions containing the desired compound were evaporated to dryness to give 32.3 mg of 5-[(2-hydroxyethyl)amino]-15,21-dimethyl-23-oxa-2,9,11,15,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,17(33),18(22),19-octaene-12,16-dione as a white solid (60%). LCMS: m/z (ESI), [M+H]⁺=559.20. ¹H NMR (DMSO-d₆, 400 MHz) δ 1.48-1.82 (3H, m), 1.98 (1H, d), 2.42 (3H, d), 2.56 (1H, s), 2.73 (1H, d), 3.10 (2H, s), 3.18-3.25 (1H, m), 3.31 (1H, s), 3.55-3.63 (6H, m), 3.66 (1H, d), 3.72 (3H, s), 4.55 (1H, t), 4.64-4.74 (2H, m), 5.47 (1H, s), 6.51 (1H, dd), 6.96 (1H, s), 7.28 (1H, d), 8.25 (1H, d), 8.41 (1H, s), 8.82 (1H, d), 12.47 (1H, s).

### Example B1

### 15,21-Dimethyl-23-oxa-2,9,11,16,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,15,17(34),18(22),19-nonaen-12-one

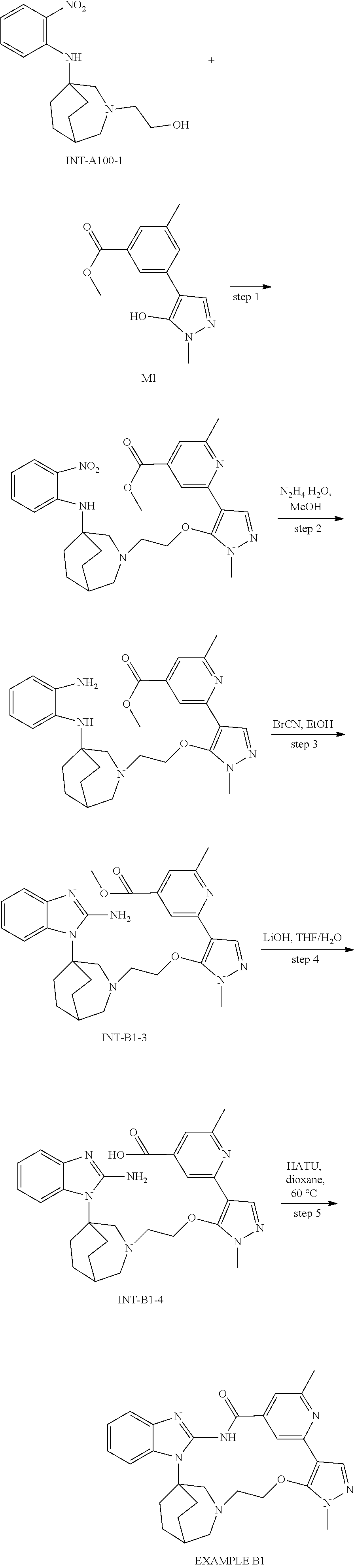

Step 1. Preparation of methyl 2-methyl-6-[1-methyl-5-(2-{1-[(2-nitrophenyl)amino]-3-azabicyclo[3.2.2]nonan-3-yl}ethoxy)pyrazol-4-yl]pyridine-4-carboxylate (INT-B1-1). To a stirred mixture of 2-{1-[(2-nitrophenyl)amino]-3-azabicyclo[3.2.2]nonan-3-yl}ethanol (250 mg, 0.81 mmol, 1 equiv) and methyl 2-(5-hydroxy-1-methylpyrazol-4-yl)-6-methylpyridine-4-carboxylate (M1, 202 mg, 0.81 mmol, 1 equiv) in toluene (100 mL) was added 2-(tributyl-lambda5-phosphanylidene)acetonitrile (1580 mg, 6.55 mmol, 8 equiv) under nitrogen atmosphere. The mixture was stirred for 4 h at 100 °C, cooled to room temperature, and concentrated under reduced pressure. The reaction was quenched with water (80 mL) and the mixture was extracted with ethyl acetate (3×150 mL). The combined organic layers were washed with brine (3×150 mL), dried over anhydrous Na₂SO₄, and concentrated under reduced pressure. The residue was purified by Prep-TLC eluted with dichloromethane/methanol (10/1) to give 420 mg of methyl 2-methyl-6-[1-methyl-5-(2-{1-[(2-nitrophenyl)amino]-3-azabicyclo[3.2.2]nonan-3-yl}ethoxy)pyrazol-4-yl]pyridine-4-carboxylate (INT-B1-1) as a yellow oil (95%). LCMS: m/z (ESI), [M+H]⁺=535.30.

Step 2. Preparation of methyl 2-[5-(2-{1-[(2-aminophenyl)amino]-3-azabicyclo[3.2.2]nonan-3-yl}ethoxy)-1-methylpyrazol-4-yl]-6-methylpyridine-4-carboxylate (INT-B1-2). To a stirred mixture of methyl 2-methyl-6-[1-methyl-5-(2-{1-[(2-nitrophenyl)amino]-3-azabicyclo[3.2.2]nonan-3-yl}ethoxy)pyrazol-4-yl]pyridine-4-carboxylate (400 mg, 0.75 mmol, 1 equiv) and RaneyNi (512 mg, 5.98 mmol, 8 equiv) in MeOH (30 mL) was added NH₂NH₂·H₂O (74 mg, 1.50 mmol, 2 equiv) at room temperature under nitrogen atmosphere. The resulting mixture was stirred for 2 h at room temperature and filtered. The filter cake was washed with methanol (3×20 mL). The resulting solution was concentrated under reduced pressure to give 360 mg methyl 2-[5-(2-{1-[(2-aminophenyl)amino]-3-azabicyclo[3.2.2]nonan-3-yl}ethoxy)-1-methylpyrazol-4-yl]-6-methylpyridine-4-carboxylate (INT-B1-2) as yellow solid. This material was used for the next reaction without further purification. LCMS: m/z (EST), [M+H]⁺=505.30.

Step 3. Preparation of methyl 2-(5-{2-[1-(2-amino-1,3-benzodiazol-1-yl)-3-azabicyclo[3.2.2]nonan-3-yl]ethoxy}-1-methylpyrazol-4-yl)-6-methylpyridine-4-carboxylate (INT-B26-3). To a stirred mixture of methyl 2-[5-(2-{1-[(2-aminophenyl)amino]-3-azabicyclo[3.2.2]nonan-3-yl}ethoxy)-1-methylpyrazol-4-yl]-6-methylpyridine-4-carboxylate (360 mg, 0.71 mmol, 1 equiv) and BrCN (113 mg, 1.06 mmol, 1.5 equiv) in ethanol (30 mL) at room temperature under nitrogen atmosphere. The resulting mixture was stirred for 1 h at room temperature and concentrated under vacuum. The residue was purified by Prep-TLC eluted with dichloromethane/methanol (15/1) to give 256 mg of methyl 2-(5-{2-[1-(2-amino-1,3-benzodiazol-1-yl)-3-azabicyclo[3.2.2]nonan-3-yl]ethoxy}-1-methylpyrazol-4-yl)-6-methylpyridine-4-carboxylate (INT-B26-3) as a pink solid (67%). LCMS: m/z (ESI), [M+H]⁺=530.35.

Step 4. Preparation of 2-(5-{2-[1-(2-amino-1,3-benzodiazol-1-yl)-3-azabicyclo[3.2.2]nonan-3-yl]ethoxy}-1-methylpyrazol-4-yl)-6-methylpyridine-4-carboxylic acid (INT-B1-4). A mixture of methyl 2-(5-{2-[1-(2-amino-1,3-benzodiazol-1-yl)-3-azabicyclo[3.2.2]nonan-3-yl]ethoxy}-1-methylpyrazol-4-yl)-6-methylpyridine-4-carboxylate (250 mg, 0.47 mmol, 1 equiv) and LiOH·H₂O (39 mg, 0.94 mmol, 2 equiv) in tetrahydrofuran (20 mL) and H₂O (4 mL) was stirred overnight at room temperature and concentrated in vacuo. The residue was purified by C18 flash chromatography using water containing 10 mmol/L NH₄HCO₃ and 0.1% NH₃—H₂O and acetonitrile as mobile phase.

Fractions containing the desired compound were evaporated to dryness to give 170 mg of 2-(5-{2-[1-(2-amino-1,3-benzodiazol-1-yl)-3-azabicyclo[3.2.2]nonan-3-yl]ethoxy}-1-methylpyrazol-4-yl)-6-methylpyridine-4-carboxylic acid (INT-B1-4) as a yellow solid (69%). LCMS: m/z (ESI), [M+H]⁺=516.15. ¹H NMR (CD₃OD, 400 MHz) δ 1.78-1.98 (7H, m), 2.43 (3H, s), 2.64 (2H, d), 2.82 (2H, t), 2.90 (2H, t), 3.16 (2H, s), 3.68 (3H, s), 4.26 (2H, t), 3.74-6.79 (1H, m), 6.89 (1H, t), 7.07-7.10 (1H, m), 7.35 (1H, d), 7.38 (1H, d), 7.73 (1H, s), 7.76 (1H, s).

Step 5. Preparation of 15,21-dimethyl-23-oxa-2,9,11,16,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,15,17(34),18(22),19-nonaen-12-one (EXAMPLE B1). To a stirred mixture of 2-(5-{2-[1-(2-amino-1,3-benzodiazol-1-yl)-3-azabicyclo[3.2.2]nonan-3-yl]ethoxy}-1-methylpyrazol-4-yl)-6-methylpyridine-4-carboxylic acid (160 mg, 0.31 mmol, 1 equiv) and N,N,N,N-tetramethyl-O-(7-azabenzotriazol-1-yl)uroniumhexafluorophospate (353 mg, 0.93 mmol, 3 equiv) in 1,4-dioxane (10 mL) was added N,N-diisopropylethylamine (160 mg, 1.24 mmol, 4 equiv) at room temperature under air atmosphere. The mixture was stirred for 3 h at 60 °C under air atmosphere. The mixture was cooled to room temperature. The reaction was quenched with water at room temperature. The resulting mixture was extracted with dichloromethane (3×200 mL). The combined organic layers were washed with brine (3×150 mL), dried over anhydrous Na₂SO₄, and concentrated under reduced pressure. The residue was purified by Prep-HPLC with XBridge Shield RP18 OBD column using water containing 10 mmol/L NH₄HCO₃ and 0.1% NH₃·H₂O and acetonitrile as mobile phase. Fractions containing the desired compound were evaporated to dryness to give 63.1 mg of 15,21-dimethyl-23-oxa-2,9,11,16,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,15,17(34),18(22),19-nonaen-12-one (EXAMPLE B1) as a white solid (40%). LCMS: m/z (ESI), [M+H]⁺=498.20. ¹H NMR (DMSO-d₆, 400 MHz) δ 1.82-2.04 (4H, m), 2.04-2.15 (3H, m), 2.55 (3H, s), 2.82 (2H, s), 2.87 (2H, t), 3.42-3.55 (4H, m), 3.77 (3H, s), 4.38 (2H, t), 7.18 (2H, dt), 7.62 (1H, d), 7.65 (1H, d), 7.89 (1H, s), 7.91 (1H, d), 8.77 (1H, s), 13.32 (1H, s).

### Example B2

### 15,21-Dimethyl-5-(morpholin-4-yl)-23-oxa-2,9,11,16,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,15,17(34),18(22),19-nonaen-12-one

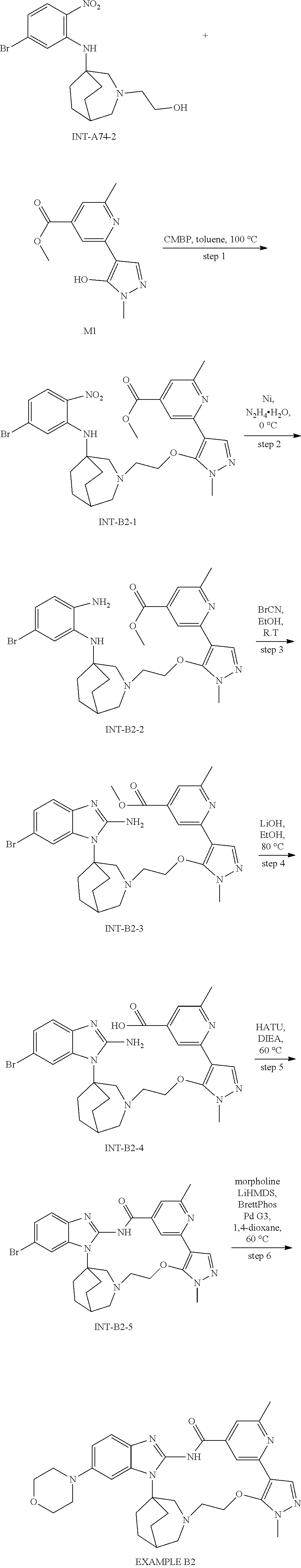

Step 1. Preparation of methyl 2-[5-(2-{1-[(5-bromo-2-nitrophenyl)amino]-3-azabicyclo[3.2.2]nonan-3-yl}ethoxy)-1-methylpyrazol-4-yl]-6-methylpyridine-4-carboxylate (INT-B2-1). A mixture of 2-{1-[(5-bromo-2-nitrophenyl)amino]-3-azabicyclo[3.2.2]nonan-3-yl}ethanol (380 mg, 0.98 mmol, 1 equiv), 2-(tributyl-lambda5-phosphanylidene)acetonitrile (1193 mg, 4.94 mmol, 5 equiv) and methyl 2-(5-hydroxy-1-methylpyrazol-4-yl)-6-methylpyridine-4-carboxylate (M1, 366 mg, 1.48 mmol, 1.5 equiv) in toluene (5 mL) was stirred for 2 h at 100 °C under nitrogen atmosphere. The mixture was cooled to room temperature. The reaction was quenched with water (100 mL) and the mixture was extracted with dichloromethane (3×100 mL). The combined organic layers were washed with brine (3×100 mL), dried over anhydrous Na₂SO₄, and concentrated under reduced pressure. The residue was purified on a Prep-TLC eluted with petroleum ether/ethyl acetate (3/1) to give 500 mg of methyl 2-[5-(2-{1-[(5-bromo-2-nitrophenyl)amino]-3-azabicyclo[3.2.2]nonan-3-yl}ethoxy)-1-methylpyrazol-4-yl]-6-methylpyridine-4-carboxylate (INT-B2-1) as an orange oil (82%). LCMS: m/z (EST), [M+H]⁺=613.15. ¹H NMR (DMSO-d₆, 400 MHz) δ 1.44-1.47 (2H, m), 1.74-1.81 (6H, m), 1.89-1.96 (1H, m), 2.48 (3H, s), 2.66-2.72 (2H, m), 2.89-2.97 (4H, m), 3.74 (3H, s), 3.84 (3H, s), 4.39 (2H, t), 6.72-6.77 (1H, m), 7.01 (1H, d), 7.33 (1H, d), 7.77 (1H, d), 7.86 (1H, s), 7.95 (1H, d), 8.06 (1H, s).

Step 2. Preparation of methyl 2-[5-(2-{1-[(2-amino-5-bromophenyl)amino]-3-azabicyclo[3.2.2]nonan-3-yl}ethoxy)-1-methylpyrazol-4-yl]-6-methylpyridine-4-carboxylate (INT-B2-2). To a stirred mixture of methyl 2-[5-(2-{1-[(5-bromo-2-nitrophenyl)amino]-3-azabicyclo[3.2.2]nonan-3-yl}ethoxy)-1-methylpyrazol-4-yl]-6-methylpyridine-4-carboxylate (480 mg, 0.78 mmol, 1 equiv) and Raney-Ni (335 mg, 3.91 mmol, 5 equiv) in methanol (5 mL) was added hydrazine hydrate (98%) (61 mg, 1.22 mmol, 1.5 equiv) at 0 °C under nitrogen atmosphere. The mixture was stirred for 2 h at 0 °C under nitrogen atmosphere.

The reaction was quenched with water (100 mL) and the mixture was extracted with dichloromethane (3×100 mL). The combined organic layers were washed with brine (3×100 mL), dried over anhydrous Na₂SO₄, and concentrated under reduced pressure. The residue was purified by Prep-TLC eluted with petroleum ether/ethyl acetate (1/1) to give 370 mg of methyl 2-[5-(2-{1-[(2-amino-5-bromophenyl)amino]-3-azabicyclo[3.2.2]nonan-3-yl}ethoxy)-1-methylpyrazol-4-yl]-6-methylpyridine-4-carboxylate (INT-B2-2) as a brown solid (81%). LCMS: m/z (ESI), [M+H]⁺=583.20. ¹H NMR (DMSO-d₆, 400 MHz) δ 1.38-1.49 (2H, m), 1.69-1.76 (4H, m), 1.85-1.88 (1H, m), 2.25-2.32 (2H, m), 2.52 (3H, s), 2.63-2.68 (2H, m), 2.78-2.83 (2H, m), 2.84-2.91 (2H, m), 3.72 (3H, s), 3.86 (3H, s), 4.03 (1H, s), 4.27-4.37 (2H, m), 4.68 (2H, s), 6.46 (2H, d), 6.58 (1H, d), 7.43 (1H, d), 7.83 (1H, d), 7.89 (1H, s).

Step 3. Preparation of methyl 2-(5-{2-[1-(2-amino-6-bromo-1,3-benzodiazol-1-yl)-3-azabicyclo[3.2.2]nonan-3-yl]ethoxy}-1-methylpyrazol-4-yl)-6-methylpyridine-4-carboxylate (INT-B2-3). A mixture of methyl 2-[5-(2-{1-[(2-amino-5-bromophenyl)amino]-3-azabicyclo[3.2.2]nonan-3-yl}ethoxy)-1-methylpyrazol-4-yl]-6-methylpyridine-4-carboxylate (350 mg, 0.60 mmol, 1 equiv) and BrCN (95 mg, 0.90 mmol, 1.5 equiv) in ethanol (2 mL) was stirred overnight at room temperature under nitrogen atmosphere. The resulting mixture was concentrated under reduced pressure. The residue was purified vy Prep-TLC eluted with petroleum ether/ethyl acetate (1/3) to give 360 mg of methyl 2-(5-{2-[1-(2-amino-6-bromo-1,3-benzodiazol-1-yl)-3-azabicyclo[3.2.2]nonan-3-yl]ethoxy}-1-methylpyrazol-4-yl)-6-methylpyridine-4-carboxylate (INT-B2-3) as an brown solid (98%). LCMS: m/z (EST), [M+H]⁺=610.15. ¹H NMR (DMSO-d₆, 400 MHz) δ 1.79-1.97 (7H, m), 2.51 (3H, s), 2.70-2.74 (2H, m), 2.79-2.87 (2H, m), 2.92-2.99 (2H, m), 3.18 (2H, s), 3.74 (3H, s), 3.84 (3H, s), 4.37 (2H, t), 6.05 (2H, s), 6.99-7.08 (2H, m), 7.41 (1H, d), 7.46 (1H, d), 7.81 (1H, d), 7.89 (1H, s).

Step 4. Preparation of lithio 2-(5-{2-[1-(2-amino-6-bromo-1,3-benzodiazol-1-yl)-3-azabicyclo[3.2.2]nonan-3-yl]ethoxy}-1-methylpyrazol-4-yl)-6-methylpyridine-4-carboxylate (INT-B2-4). A mixture of methyl 2-(5-{2-[1-(2-amino-6-bromo-1,3-benzodiazol-1-yl)-3-azabicyclo[3.2.2]nonan-3-yl]ethoxy}-1-methylpyrazol-4-yl)-6-methylpyridine-4-carboxylate (340 mg, 0.55 mmol, 1 equiv) and LiOH (16 mg, 0.67 mmol, 1.2 equiv) in ethanol (2 mL) was stirred for 2 h at 80 °C under nitrogen atmosphere. The mixture was cooled to room temperature and concentrated under reduced pressure to give 2-(5-{2-[1-(2-amino-6-bromo-1,3-benzodiazol-1-yl)-3-azabicyclo[3.2.2]nonan-3-yl]ethoxy}-1-methylpyrazol-4-yl)-6-methylpyridine-4-carboxylic acid (INT-B2-4) as a white solid. The crude product was used for the next reaction directly without further purification. LCMS: m/z (EST), [M+H]⁺=594.45.

Step 5. Preparation of 5-bromo-15,21-dimethyl-23-oxa-2,9,11,16,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,15,17(34),18(22),19-nonaen-12-one (INT-B2-5). A mixture of 2-(5-{2-[1-(2-amino-6-bromo-1,3-benzodiazol-1-yl)-3-azabicyclo[3.2.2]nonan-3-yl]ethoxy}-1-methylpyrazol-4-yl)-6-methylpyridine-4-carboxylic acid (330 mg crude, ~0.5 mmol, ~1 equiv), N,N,N,N-tetramethyl-O-(7-azabenzotriazol-1-yl)uronium hexafluorophospate (417 mg, 1.10 mmol, 2 equiv) and N,N-diisopropylethylamine (213 mg, 1.65 mmol, 3 equiv) in 1,4-dioxane (5 mL) was stirred for 2 h at 60 °C under nitrogen atmosphere. The mixture was cooled to room temperature. The reaction was quenched with water (50 mL) and the mixture was extracted with ethyl acetate (3×50 mL). The combined organic layers were washed with brine (3×150 mL), dried over anhydrous Na₂SO₄, and concentrated under reduced pressure. The residue was purified by trituration with petroleum ether (10 mL) to give 280 mg of 5-bromo-15,21-dimethyl-23-oxa-2,9,11,16,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,15,17(34),18(22),19-nonaen-12-one (INT-B2-5) as a yellow solid (88%). LCMS: m/z (EST), [M+H]⁺=578.05.

Step 6. Preparation of 15,21-dimethyl-5-(morpholin-4-yl)-23-oxa-2,9,11,16,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,15,17(34),18(22),19-nonaen-12-one (EXAMPLE B2). To a stirred mixture of 5-bromo-15,21-dimethyl-23-oxa-2,9,11,16,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,15,17(34),18(22),19-nonaen-12-one (100 mg, 0.17 mmol, 1 equiv), Brettphos Pd G₃ (47 mg, 0.05 mmol, 0.3 equiv), and morpholine (52.89 mg, 0.605 mmol, 3.5 equiv) in 1,4-dioxane (10 mL) was added LiHMDS (2 mL, 2.00 mmol, 11.53 equiv) dropwise at room temperature under nitrogen atmosphere. The resulting mixture was stirred for 1 h at 60 °C under nitrogen atmosphere and cooled to room temperature. The reaction was quenched with sat. NH₄Cl (aq.) (50 mL) and the mixture was extracted with CH₂Cl₂ (3×50 mL). The combined organic layers were washed with brine (3×50 mL), dried over anhydrous Na₂SO₄, and concentrated under reduced pressure. The residue was purified by prep-HPLC with XBridge Prep OBD C18 column using water containing 10 mmol/L NH₄HCO₃ and acetonitrile as mobile phase. Fractions containing the desired compound were evaporated to dryness to give 14.4 mg of 15,21-dimethyl-5-(morpholin-4-yl)-23-oxa-2,9,11,16,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,15,17(34),18(22),19-nonaen-12-one (EXAMPLE B2) as a yellow solid (14%). LCMS: m/z (EST), [M+H]⁺=583.40. ¹H NMR (DMSO-d₆, 400 MHz) δ 1.88-1.99 (4H, m), 2.03-2.11 (3H, m), 2.54 (3H, s), 2.78-2.90 (4H, m), 3.04-3.10 (4H, m), 3.42-3.50 (4H, m), 3.75-3.80 (7H, m), 4.36-4.40 (2H, m), 6.95 (1H, dd), 7.35 (1H, s), 7.51 (1H, d), 7.61 (1H, d), 7.89 (1H, s), 8.76 (1H, s), 13.21 (1H, s).

### Example B3

### 15,21-dimethyl-5-(4-methylpiperazin-1-yl)-23-oxa-2,9,11,16,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,15,17(34),18(22),19-nonaen-12-one

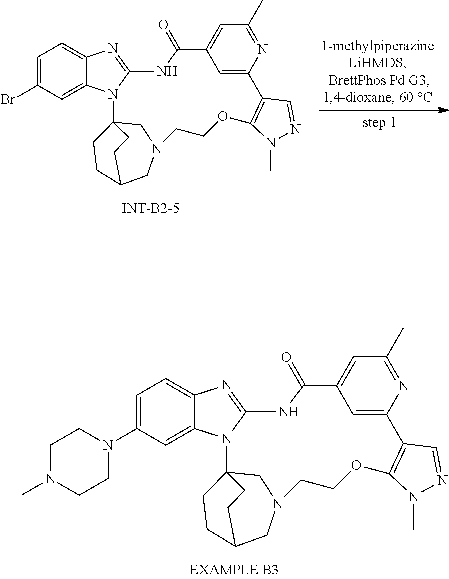 [SMILES: Cc1cc2cc(n1)-c1cnn(C)c1OCCN1CC3CCC(CC3)(C1)n1c(nc3ccc(Br)cc31)NC2=O ; Cc1cc2cc(n1)-c1cnn(C)c1OCCN1CC3CCC(CC3)(C1)n1c(nc3ccc(N4CCN(C)CC4)cc31)NC2=O]

Step 1. Preparation of 15,21-dimethyl-5-(4-methylpiperazin-1-yl)-23-oxa-2,9,11,16,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,15,17(34),18(22),19-nonaen-12-one (EXAMPLE B3). To a stirred mixture of 5-bromo-15,21-dimethyl-23-oxa-2,9,11,16,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,15,17(34),18(22),19-nonaen-12-one (100 mg, 0.17 mmol, 1 equiv), BrettPhos Pd G₃ (550 mg, 0.60 mmol, 3.5 equiv), and 1-methylpiperazine (60.81 mg, 0.605 mmol, 3.5 equiv) in 1,4-dioxane (10 mL) was added LiHMDS (2 mL, 2.000 mmol, 11.53 equiv) dropwise at room temperature under nitrogen atmosphere. The resulting mixture was stirred for 1 h at 60 °C under nitrogen atmosphere and cooled to room temperature. The reaction was quenched with sat. NH₄Cl (aq.) (50 mL) and the mixture was extracted with CH₂Cl₂ (3×50 mL). The combined organic layers were washed with brine (3×50 mL), dried over anhydrous Na₂SO₄, and concentrated under reduced pressure. The residue was purified by prep-HPLC with XBridge Shield RP18 OBD column using water containing 10 mmol/L NH₄HCO₃ and acetonitrile as mobile phase. Fractions containing the desired compound were evaporated to dryness to give 13.7 mg of 15,21-dimethyl-5-(4-methylpiperazin-1-yl)-23-oxa-2,9,11,16,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,15,17(34),18(22),19-nonaen-12-one (EXAMPLE B3) as a yellow solid (13%). LCMS: m/z (ESI), [M+H]⁺=596.40. ¹H NMR (DMSO-d₆, 400 MHz) δ 1.84-2.01 (4H, m), 2.03-2.10 (3H, m), 2.24 (3H, s), 2.54 (4H, s), 2.79-2.84 (2H, m), 2.85-2.90 (2H, m), 3.10 (4H, t), 3.28-3.36 (3H, m), 3.42-3.50 (4H, m), 3.76 (3H, s), 4.37 (2H, d), 6.94 (1H, dd), 7.34 (1H, s), 7.49 (1H, d), 7.61 (1H, d), 7.89 (1H, s), 8.76 (1H, s), 13.20 (1H, s).

### Example B4

### 15,21-Dimethyl-23-oxa-2,9,11,16,20,21,26-heptaazahexacyclo[24.4.1.1\hat{ }{13,17}.0\hat{ }{2,10}.0\hat{ }{3,8}.0\hat{ }{18,22}]dotriaconta-3,5,7,9,13(32),14,16,18(22),19-nonaen-12-one

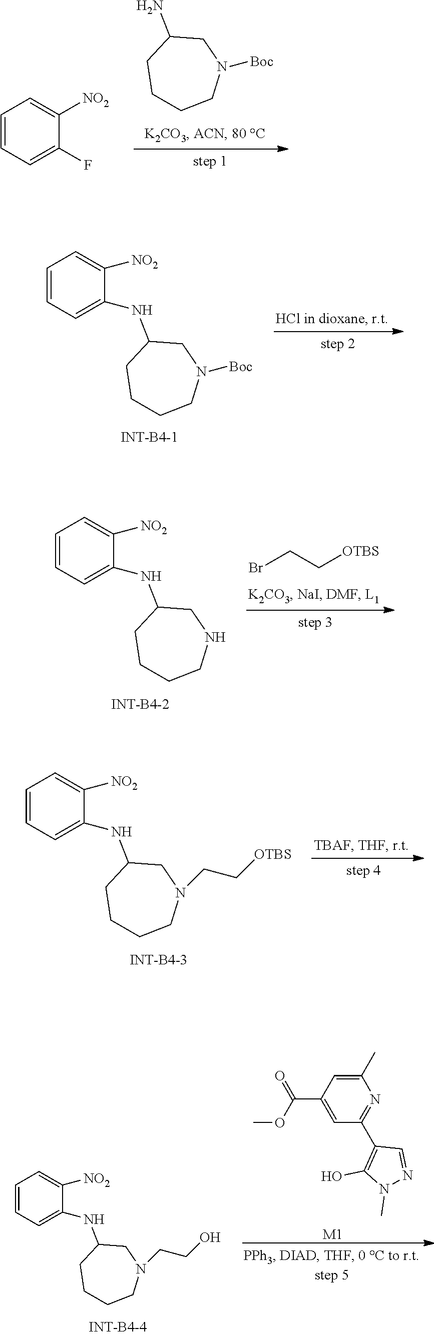

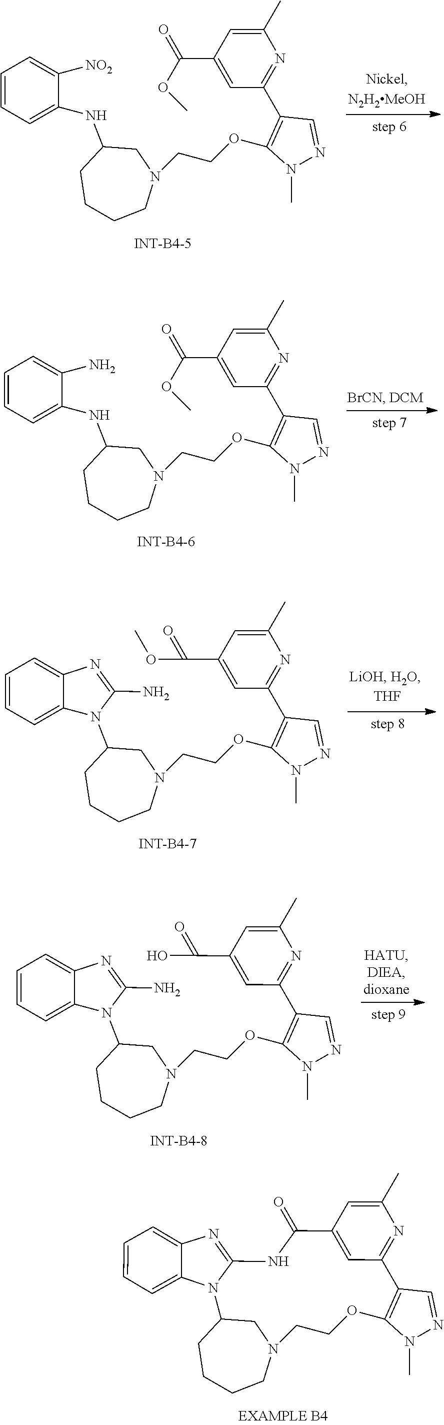 [SMILES: COC(=O)c1cc(C)nc(-c2cnn(C)c2OCCN2CCCCC(Nc3ccccc3[N+](=O)[O-])C2)c1 ; COC(=O)c1cc(C)nc(-c2cnn(C)c2OCCN2CCCCC(Nc3ccccc3N)C2)c1 ; COC(=O)c1cc(C)nc(-c2cnn(C)c2OCCN2CCCCC(n3c(N)nc4ccccc43)C2)c1 ; Cc1cc(C(=O)O)cc(-c2cnn(C)c2OCCN2CCCCC(n3c(N)nc4ccccc43)C2)n1 ; Cc1cc2cc(n1)-c1cnn(C)c1OCCN1CCCCC(C1)n1c(nc3ccccc31)NC2=O]

Step 1. Preparation of tert-butyl 3-[(2-nitrophenyl)amino]azepane-1-carboxylate (INT-B4-1). A mixture of O-fluoronitrobenzene (5.00 g, 35.44 mmol, 1 equiv), K₂CO₃ (9.79 g, 70.87 mmol, 2 equiv) and tert-butyl 3-aminoazepane-1-carboxylate (7.59 g, 35.44 mmol, 1 equiv) in acetonitrile (80 mL) was stirred for 3 h at 80 °C under nitrogen atmosphere. The mixture was cooled to room temperature. The reaction was quenched with water (200 mL) and the mixture was extracted with ethyl acetate (3×200 mL). The combined organic layers were washed with brine (3×200 mL), dried over anhydrous Na₂SO₄, and concentrated under reduced pressure. The residue was purified by silica gel chromatography eluted with petroleum ether/ethyl acetate (21/1) to give 9 g of tert-butyl 3-[(2-nitrophenyl)amino]azepane-1-carboxylate (INT-B4-1) as a reddish oil (75%). LCMS: m/z (ESI), [M+H]⁺=336.15. ¹H NMR (400 MHz, DMSO-d₆) δ 1.38 (9H, s), 1.49-1.65 (1H, m), 1.62-1.75 (3H, m), 1.75-1.82 (1H, m), 1.96-2.01 (1H, m), 3.37-3.50 (2H, m), 3.61-3.69 (1H, m), 3.83-4.03 (2H, m), 6.66-6.76 (1H, m), 7.21-7.25 (1H, m), 7.51-7.61 (1H, m), 8.01-8.13 (1H, m), 8.20 (1H, d).

Step 2. Preparation of N-(2-nitrophenyl)azepan-3-amine (INT-B4-2). To a stirred mixture of tert-butyl 3-[(2-nitrophenyl)amino]azepane-1-carboxylate (4.00 g, 11.93 mmol, 1 equiv) in dichloromethane (12 mL) was added HCl in 1,4-dioxane (60 mL) slowly at room temperature. The resulting mixture was stirred for 1.5 h at room temperature. The mixture was concentrated under reduced pressure. The residue was treated with saturated NaHCO₃ (aq.) (100 mL) and extracted with ethyl acetate (3×100 mL). The combined organic layers were washed with brine (3×100 mL), dried over anhydrous Na₂SO₄, and concentrated under reduced pressure to give 2.8 g of N-(2-nitrophenyl)azepan-3-amine (INT-B4-2) as a yellow oil. This material was used for the next reaction without further purification. LCMS: m/z (ESI), [M+H]⁺=236.10.

Step 3. Preparation of 1-{2-[(tert-butyldimethylsilyl)oxy]ethyl}-N-(2-nitrophenyl)azepan-3-amine (INT-B4-3). A mixture of freshly prepared N-(2-nitrophenyl)azepan-3-amine (2.8 g, ~1 equiv), (2-bromoethoxy)(tert-butyl)dimethylsilane (6.39 g, 26.69 mmol, 2 equiv), K₂CO₃ (3.69 g, 26.69 mmol, 2 equiv), and KI (4.43 g, 26.69 mmol, 2 equiv) in DMF (80 mL) was stirred for 2 h at 60 °C under nitrogen atmosphere. The mixture was cooled to room temperature. The reaction was quenched with water (100 mL) and the mixture was extracted with ethyl acetate (3×100 mL). The combined organic layers were washed with brine (3×100 mL), dried over anhydrous Na₂SO₄, and concentrated under reduced pressure. The residue was purified by prep-TLC eluted with petroleum ether/ethyl acetate (19/1) to give 5.2 g of 1-{2-[(tert-butyldimethylsilyl)oxy]ethyl}-N-(2-nitrophenyl)azepan-3-amine (INT-B4-3) as a yellow oil (99%). LCMS: m/z (ESI), [M+H]=394.20. ¹H NMR (400 MHz, DMSO-d₆) δ 0.03 (6H, s), 0.81 (9H, s), 1.45-1.63 (3H, m), 1.74-1.81 (3H, m), 2.57-2.77 (3H, m), 2.78-2.95 (3H, m), 3.29 (1H, t), 3.63-3.84 (3H, m), 6.65-6.71 (1H, m), 7.05 (1H, d), 7.51-7.58 (1H, m), 8.07-8.13 (1H, m), 8.74 (1H, d).

Step 4. Preparation of 2-{3-[(2-nitrophenyl)amino]azepan-1-yl}ethanol (INT-B4-4). To a stirred mixture of 1-{2-[(tert-butyldimethylsilyl)oxy]ethyl}-N-(2-nitrophenyl)azepan-3-amine (3 g, 7.62 mmol, 1 equiv) in tetrahydrofuran (50 mL) was added tetrabutylammonium fluoride in tetrahydrofuran (12 mL) at room temperature under nitrogen atmosphere. The resulting mixture was stirred for 1 h at room temperature. The reaction was quenched with water (100 mL) and the mixture was extracted with dichloromethane (3×100 mL). The combined organic layers were washed with brine (3×100 mL), dried over anhydrous Na₂SO₄, and concentrated under reduced pressure. The residue was purified by Prep-TLC eluted with petroleum ether/ethyl acetate (1/1) to give 1.5 g of 2-{3-[(2-nitrophenyl)amino]azepan-1-yl}ethanol (INT-B4-4) as a yellow oil (70%). LCMS: m/z (ESI), [M+H]⁺=280.10.

Step 5. Preparation of methyl 2-methyl-6-[1-methyl-5-(2-{3-[(2-nitrophenyl)amino]azepan-1-yl}ethoxy)pyrazol-4-yl]pyridine-4-carboxylate (INT-B4-5). To a stirred mixture of 2-{3-[(2-nitrophenyl)amino]azepan-1-yl}ethanol (1.5 g, 5.370 mmol, 1 equiv) and triphenylphosphine (4.30 g, 16.38 mmol, 3.05 equiv) in tetrahydrofuran (30 mL) were added methyl 2-(5-hydroxy-1-methylpyrazol-4-yl)-6-methylpyridine-4-carboxylate (M1, 1.46 g, 5.91 mmol, 1.1 equiv) and diisopropyl azodicarboxylate (2.71 g, 13.43 mmol, 2.5 equiv) at 0 °C under nitrogen atmosphere. The resulting mixture was stirred for 2 h at room temperature. The reaction was quenched with water (300 mL) and the mixture was extracted with dichloromethane (3×300 mL). The combined organic layers were washed with brine (3×300 mL), dried over anhydrous Na₂SO₄, and concentrated under reduced pressure. The residue was purified by reverse flash chromatography eluted with 10% to 50% acetonitrile in water to give 1.6 g of methyl 2-methyl-6-[1-methyl-5-(2-{3-[(2-nitrophenyl)amino]azepan-1-yl}ethoxy)pyrazol-4-yl]pyridine-4-carboxylate (INT-B4-5) as a yellow oil (59%). LCMS: m/z (ESI), [M+H]⁺=509.25. ¹H NMR (400 MHz, DMSO-d₆) δ 1.44-1.80 (6H, m), 2.48-2.54 (2H, m), 2.71-2.81 (1H, m), 2.85-2.92 (2H, m), 2.94-3.14 (3H, m), 3.70 (3H, s), 3.86 (3H, s), 3.92 (1H, s), 4.28-4.35 (2H, m), 6.63-6.68 (1H, m), 6.96-7.03 (1H, m), 7.38 (1H, d), 7.46-7.51 (1H, m), 7.80-7.83 (1H, m), 7.88 (1H, s), 8.00-8.05 (1H, m), 8.62 (1H, d).

Step 6. Preparation of methyl 2-[5-(2-{3-[(2-aminophenyl)amino]azepan-1-yl}ethoxy)-1-methylpyrazol-4-yl]-6-methylpyridine-4-carboxylate (INT-B4-6). To a stirred mixture of methyl 2-methyl-6-[1-methyl-5-(2-{3-[(2-nitrophenyl)amino]azepan-1-yl}ethoxy)pyrazol-4-yl]pyridine-4-carboxylate (1.00 g, 1.97 mmol, 1 equiv) and Raney Ni (340 mg, 3.93 mmol, 2 equiv) in MeOH (30 mL) was added NH₂NH₂·H₂O (295 mg, 5.90 mmol, 3 equiv) at 0 °C under nitrogen atmosphere. The resulting mixture was stirred for 1 h at room temperature. The mixture was filtered and the filter cake was washed with dichloromethane (3×30 mL). The resulting solution was concentrated under reduced pressure. The residue was purified by Prep-TLC eluted with dichloromethane/methanol (30/1) to give 500 mg of methyl 2-[5-(2-{3-[(2-aminophenyl)amino]azepan-1-yl}ethoxy)-1-methylpyrazol-4-yl]-6-methylpyridine-4-carboxylate (INT-B4-6) as a reddish oil (53%). LCMS: m/z (EST), [M+H]⁺=479.25.

Step 7. Preparation of methyl 2-(5-{2-[3-(2-amino-1,3-benzodiazol-1-yl)azepan-1-yl]ethoxy}-1-methylpyrazol-4-yl)-6-methylpyridine-4-carboxylate (INT-B4-7). A mixture of methyl 2-[5-(2-{3-[(2-aminophenyl)amino]azepan-1-yl}ethoxy)-1-methylpyrazol-4-yl]-6-methylpyridine-4-carboxylate (490 mg, 1.02 mmol, 1 equiv) and cyanogen bromide (108 mg, 1.02 mmol, 1 equiv) in dichloromethane (5 mL) was stirred for 2 h at room temperature under nitrogen atmosphere. The mixture was concentrated under reduced pressure and the residue was purified by Prep-TLC eluted with dichloromethane/methanol (30/1) to give 490 mg of methyl 2-(5-{2-[3-(2-amino-1,3-benzodiazol-1-yl)azepan-1-yl]ethoxy}-1-methylpyrazol-4-yl)-6-methylpyridine-4-carboxylate (INT-B4-7) as a yellow oil (95%). LCMS: m/z (ESI), [M+H]⁺=504.25. ¹H NMR (400 MHz, DMSO-d₆) δ 1.50-1.56 (1H, m), 1.61-1.70 (1H, m), 1.73-1.99 (2H, m), 2.52 (5H, s), 2.93-3.03 (1H, m), 3.17 (1H, s), 3.26-3.32 (3H, m), 3.70 (3H, s), 3.84 (3H, s), 4.28 (2H, s), 4.62 (1H, s), 6.96 (1H, s), 7.00 (1H, d), 7.20 (1H, d), 7.39 (1H, s), 7.44 (1H, d), 7.83-7.88 (1H, m), 7.91 (1H, s).

Step 8. Preparation of 2-(5-{2-[3-(2-amino-1,3-benzodiazol-1-yl)azepan-1-yl]ethoxy}-1-methylpyrazol-4-yl)-6-methylpyridine-4-carboxylic acid (INT-B4-8). A mixture of methyl 2-(5-{2-[3-(2-amino-1,3-benzodiazol-1-yl)azepan-1-yl]ethoxy}-1-methylpyrazol-4-yl)-6-methylpyridine-4-carboxylate (480 mg, 0.95 mmol, 1 equiv) and LiOH (45 mg, 1.91 mmol, 2 equiv) in tetrahydrofuran (6 mL) and H₂O (1.5 mL) was stirred for 1 h at room temperature. The mixture was concentrated under reduced pressure and the residue was purified by reverse flash chromatography eluted with 10% to 30% acetonitrile in water to give 125 mg of 2-(5-{2-[3-(2-amino-1,3-benzodiazol-1-yl)azepan-1-yl]ethoxy}-1-methylpyrazol-4-yl)-6-methylpyridine-4-carboxylic acid (INT-B4-8) as a white solid (27%). LCMS: m/z (ESI), [M+H]⁺=490.15.

Step 9. Preparation of 15,21-dimethyl-23-oxa-2,9,11,16,20,21,26-heptaazahexacyclo[24.4.1.1\hat{ }{13,17}.0\hat{ }{2,10}.0\hat{ }{3,8}.0\hat{ }{18,22}]dotriaconta-3,5,7,9,13(32),14,16,18(22),19-nonaen-12-one (EXAMPLE B4). A mixture of 2-(5-{2-[3-(2-amino-1,3-benzodiazol-1-yl)azepan-1-yl]ethoxy}-1-methylpyrazol-4-yl)-6-methylpyridine-4-carboxylic acid (120 mg, 0.25 mmol, 1 equiv), N,N-diisopropylethylamine (95 mg, 0.74 mmol, 3 equiv) and N,N,N,N-tetramethyl-O-(7-azabenzotriazol-1-yl)uronium hexafluorophospate (140 mg, 0.37 mmol, 1.5 equiv) in 1,4-dioxane (6 mL) was stirred for 1 h at room temperature under nitrogen atmosphere. The mixture was concentrated under reduced pressure. The residue was purified by Prep-HPLC using water containing 10 mmol/L NH₄HCO₃ and acetonitrile as mobile phase. Fractions containing the desired compound were evaporated to give 60.9 mg of 15,21-dimethyl-23-oxa-2,9,11,16,20,21,26-heptaazahexacyclo[24.4.1.1\hat{ }{13,17}.0\hat{ }{2,10}.0\hat{ }{3,8}.0\hat{ }{18,22}]dotriaconta-3,5,7,9,13(32),14,16,18(22),19-nonaen-12-one (EXAMPLE B4) as a white solid (52%). LCMS: m/z (EST), [M+H]⁺=472.25. ¹H NMR (DMSO-d₆, 400 MHz) δ 1.63-2.02 (6H, m), 2.57 (3H, s), 2.62-2.76 (1H, m), 2.79-2.85 (1H, m), 2.88-3.01 (3H, m), 3.10-3.17 (1H, m), 3.76 (3H, s), 4.04 (1H, br s), 4.38 (1H, br s), 5.15 (1H, br s), 7.19-7.30 (2H, m), 7.52-7.59 (2H, m), 7.68 (1H, d), 7.91 (1H, s), 8.62 (1H, s), 12.72 (1H, s).

### Example B6

### 11-Cyclopropyl-26-methyl-8-(2,2,2-trifluoroethyl)-11H,51H-11-oxa-4,8-diaza-5(2,1)-benzo[d]imidazola-2(2,4)-pyridina-1(4,5)-pyrazolacycloundecaphan-3-one

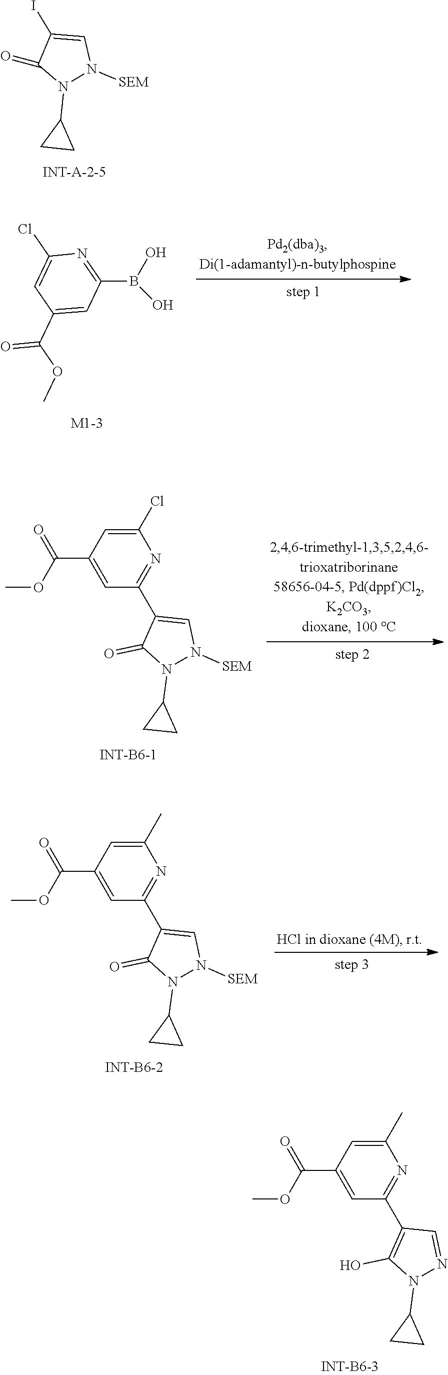 [SMILES: C[Si](C)(C)CCOCn1cc(I)c(=O)n1C1CC1 ; COC(=O)c1cc(Cl)nc(B(O)O)c1 ; COC(=O)c1cc(Cl)nc(-c2cn(COCC[Si](C)(C)C)n(C3CC3)c2=O)c1 ; COC(=O)c1cc(C)nc(-c2cn(COCC[Si](C)(C)C)n(C3CC3)c2=O)c1 ; COC(=O)c1cc(C)nc(-c2cnn(C3CC3)c2O)c1]

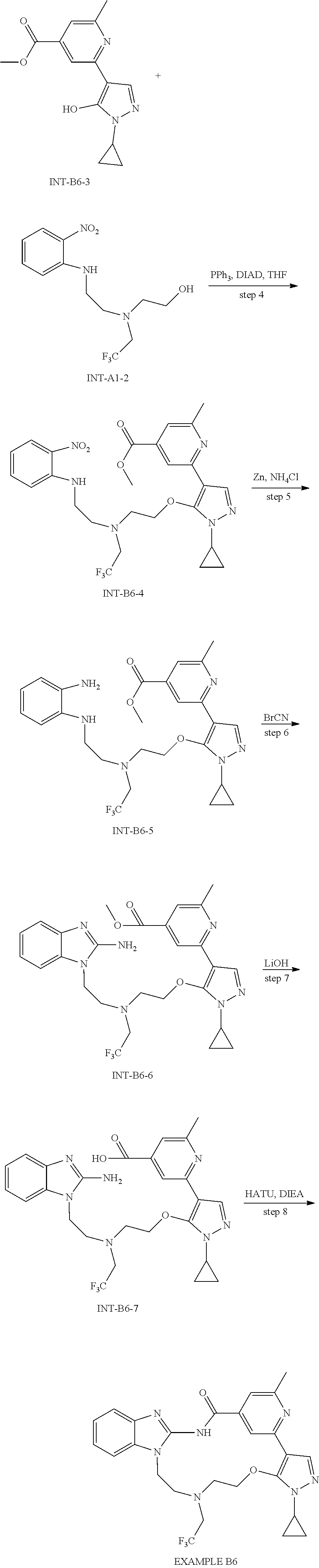

Step 1. Preparation of methyl 2-chloro-6-(2-cyclopropyl-3-oxo-1-((2-(trimethylsilyl)ethoxy)methyl)-2,3-dihydro-1H-pyrazol-4-yl)isonicotinate (INT-B6-1). To a stirred mixture of 2-cyclopropyl-4-iodo-1-{[2-(trimethylsilyl)ethoxy]methyl}pyrazol-3-one (2.3 g, 6.04 mmol, 1 equiv) and freshly prepared (6-chloro-4-(methoxycarbonyl)pyridin-2-yl)boronic acid (M1-3, 9.0 g of the crude material, −30 mmol, ~5 equiv) in toluene (20 mL) and water (5 mL) were added Pd₂(dba)₃ (1.1 g, 1.21 mmol, 0.2 equiv), di(1-adamantyl)-n-butylphosphine (0.1 g, 1.21 mmol, 0.2 equiv), and Cs₂CO₃ (4.9 g, 15.12 mmol, 2.5 equiv) at room temperature under nitrogen atmosphere. The resulting mixture was stirred for 2 h at 80 °C and cooled to room temperature. The reaction was quenched with water (300 mL) and the mixture was extracted with ethyl acetate (3×200 mL). The combined organic layers were washed with brine (3×100 mL), dried over anhydrous Na₂SO₄, and concentrated under reduced pressure. The residue was purified by silica gel column chromatography eluted with petroleum ether/ethyl acetate (1/1) to give 1.8 g of methyl 2-chloro-6-(2-cyclopropyl-3-oxo-1-{[2-(trimethylsilyl)ethoxy]methyl}pyrazol-4-yl)pyridine-4-carboxylate (INT-B6-1) as a yellow oil (70%). ¹H NMR (DMSO-d₆, 400 MHz) δ −0.05 (9H, s), 0.78-0.83 (2H, m), 0.87-0.90 (4H, m), 3.30 (2H, d), 3.94-3.98 (3H, m), 2.05-2.10 (1H, m), 5.41 (2H, s), 7.55-7.51 (1H, m), 8.66-8.70 (2H, m).

Step 2. Preparation of methyl 2-(2-cyclopropyl-3-oxo-1-((2-(trimethylsilyl)ethoxy)methyl)-2,3-dihydro-1H-pyrazol-4-yl)-6-methylisonicotinate (INT-B6-2). To a stirred mixture of methyl 2-chloro-6-(2-cyclopropyl-3-oxo-1-{[2-(trimethylsilyl)ethoxy]methyl}pyrazol-4-yl)pyridine-4-carboxylate (1.8 g, 4.24 mmol, 1 equiv) and trimethyl-1,3,5,2,4,6-trioxatriborinane (3.2 g, 25.47 mmol, 6.0 equiv) in 1,4-dioxane (25 mL) were added PCy₃·HBF₄ (0.5 g, 1.27 mmol, 0.3 equiv), Pd(dppf)Cl₂ CH₂Cl₂ (0.6 g, 0.84 mmol, 0.2 equiv), and K₂CO₃ (1.1 g, 8.49 mmol, 2.0 equiv) at room temperature under nitrogen atmosphere. The resulting mixture was stirred overnight at 100 °C and cooled to room temperature. The reaction was quenched with water (50 mL) and the mixture was extracted with ethyl acetate (3×100 mL). The combined organic layers were washed with brine (3×200 mL), dried over anhydrous Na₂SO₄, and concentrated under reduced pressure.

The residue was purified on a silica gel column eluted with petroleum ether/ethyl acetate (1/1) to give 1.3 g of methyl 2-(2-cyclopropyl-3-oxo-1-((2-(trimethylsilyl)ethoxy)methyl)-2,3-dihydro-1H-pyrazol-4-yl)-6-methylisonicotinate (INT-B6-2) as a brown solid (75%). ¹H NMR (DMSO-d₆, 400 MHz) δ −0.05 (9H, s), 0.80-0.84 (2H, m), 1.00-1.05 (4H, m), 2.53 (3H, s), 2.74-2.80 (1H, m), 3.45-3.50 (2H, m), 3.89 (3H, s), 5.38 (2H, s), 7.45 (1H, d), 8.55 (1H, d), 8.59 (1H, s).

Step 3. Preparation of methyl 2-(1-cyclopropyl-5-hydroxypyrazol-4-yl)-6-methylpyridine-4-carboxylate (INT-B6-3). A mixture of methyl 2-(2-cyclopropyl-3-oxo-1-{[2-(trimethylsilyl)ethoxy]methyl}pyrazol-4-yl)-6-methylpyridine-4-carboxylate (1.3 g, 3.22 mmol, 1 equiv) and HCl in 1,4-dioxane (10 mL) was stirred for 2 h at room temperature under nitrogen atmosphere and concentrated under vacuum. The residue was treated with CH₂Cl₂ (20 mL) and saturated NaHCO₃ (aq.) (50 mL). The resulting mixture was extracted with dichloromethane (3×100 mL). The combined organic layers were washed with brine (3×50 mL), dried over anhydrous Na₂SO₄, and concentrated under reduced pressure. The residue was loaded onto a silica gel column and eluted with dichloromethane/methanol (10/1) to give 844 mg of methyl 2-(1-cyclopropyl-5-hydroxypyrazol-4-yl)-6-methylpyridine-4-carboxylate (INT-B6-3) as a brown solid (95%). ¹H NMR (DMSO-d₆, 400 MHz) δ 0.88-1.00 (4H, m), 1.24 (1H, s), 2.57 (3H, s), 3.91 (3H, s), 7.33 (1H, s), 7.91 (2H, s).

Step 4. Preparation of methyl 2-(1-cyclopropyl-5-(2-((2-((2-nitrophenyl)amino)ethyl)(2,2,2-trifluoroethyl)amino)ethoxy)-1H-pyrazol-4-yl)-6-methylisonicotinate (INT-B6-4). To a stirred mixture of methyl 2-(1-cyclopropyl-5-hydroxypyrazol-4-yl)-6-methylpyridine-4-carboxylate (142 mg, 0.52 mmol, 1.0 equiv) and triphenylphosphine (409 mg, 1.56 mmol, 3.0 equiv) in tetrahydrofuran (10 mL) were added diisopropyl azodicarboxylate (315 mg, 1.56 mmol, 3.0 equiv) and 2-({2-[(2-nitrophenyl)amino]ethyl}(2,2,2-trifluoroethyl)amino)ethanol (INT-A1-2, 400 mg, 1.30 mmol, 2.5 equiv) at 0 °C under nitrogen atmosphere. The resulting mixture was stirred for 2 h. The reaction was quenched with water (50 mL) and the mixture was extracted with ethyl acetate (3×50 mL). The combined organic layers were washed with brine (3×50 mL), dried over anhydrous Na₂SO₄, and concentrated under reduced pressure. The residue was purified by silica gel column eluted with CH₂Cl₂/methanol (10/1) to give 540 mg of methyl 2-(1-cyclopropyl-5-(2-((2-((2-nitrophenyl)amino)ethyl)(2,2,2-trifluoroethyl)amino)ethoxy)-1H-pyrazol-4-yl)-6-methylisonicotinate (INT-B6-4) as a yellow oil (57%). ¹H NMR (DMSO-d₆, 400 MHz) δ 0.91-0.99 (2H, m), 1.02-1.11 (2H, m), 2.53 (3H, s), 3.01-3.07 (2H, m), 3.39-3.43 (4H, m), 3.51-3.67 (3H, m), 3.88 (3H, s), 4.32 (2H, t), 6.61-6.66 (1H, m), 7.02-7.08 (1H, m), 7.38-7.54 (2H, m), 7.83-7.78 (2H, m), 8.03-8.07 (1H, m), 8.22 (1H, t).

Step 5. Preparation of methyl 2-{5-[2-({2-[(2-aminophenyl)amino]ethyl}(2,2,2-trifluoroethyl)amino)ethoxy]-1-cyclopropylpyrazol-4-yl}-6-methylpyridine-4-carboxylate (INT-B6-5). To a stirred mixture of methyl 2-{1-cyclopropyl-5-[2-({2-[(2-nitrophenyl)amino]ethyl}(2,2,2-trifluoroethyl)amino)ethoxy]pyrazol-4-yl}-6-methylpyridine-4-carboxylate (500 mg, 0.88 mmol, 1 equiv) and Zn (464 mg, 7.11 mmol, 8 equiv) in tetrahydrofuran (10 mL) and H₂O (2.5 mL) was added NH₄Cl (475 mg, 8.89 mmol, 10 equiv) at room temperature under nitrogen atmosphere. The mixture was stirred for 2 h at that temperature. The reaction was quenched with water (50 mL) and the mixture was extracted with dichloromethane (3×50 mL). The combined organic layers were washed with brine (3×50 mL), dried over anhydrous Na₂SO₄, and concentrated under reduced pressure. The residue was purified by a silica gel column eluted with CH₂Cl₂/methanol (5/1) to give 190 mg of methyl 2-{5-[2-({2-[(2-aminophenyl)amino]ethyl}(2,2,2-trifluoroethyl)amino)ethoxy]-1-cyclopropylpyrazol-4-yl}-6-methylpyridine-4-carboxylate (INT-B6-5) as a yellow oil (40%). ¹H NMR (DMSO-d₆, 400 MHz) δ 0.92-1.01 (2H, m), 1.03-1.07 (2H, m), 2.55 (3H, s), 3.00 (2H, t), 3.14 (2H, s), 3.48-3.52 (2H, m), 3.58-3.63 (1H, m), 3.89 (3H, s), 3.98-4.04 (1H, m), 4.29-4.31 (2H, m), 4.40 (2H, s), 6.37-6.49 (3H, m), 6.48-6.54 (1H, m), 7.49 (1H, d), 7.81-7.91 (2H, m).

Step 6. Preparation of methyl 2-[5-(2-{[2-(2-amino-1,3-benzodiazol-1-yl)ethyl](2,2,2-trifluoroethyl)amino}ethoxy)-1-cyclopropylpyrazol-4-yl]-6-methylpyridine-4-carboxylate (INT-B6-6). A mixture of methyl 2-{5-[2-({2-[(2-aminophenyl)amino]ethyl}(2,2,2-trifluoroethyl)amino)ethoxy]-1-cyclopropylpyrazol-4-yl}-6-methylpyridine-4-carboxylate (170 mg, 0.31 mmol, 1 equiv) and BrCN (37 mg, 0.35 mmol, 1.1 equiv) in ethanol (5 mL) was stirred for 4 h at room temperature under nitrogen atmosphere. The mixture was concentrated in vacuo. The residue was purified by Prep-TLC eluted with CH₂Cl₂/methanol (10/1) to give 125 mg of methyl 2-[5-(2-{[2-(2-amino-1,3-benzodiazol-1-yl)ethyl](2,2,2-trifluoroethyl)amino}ethoxy)-1-cyclopropylpyrazol-4-yl]-6-methylpyridine-4-carboxylate (INT-B6-6) as a brown oil (70%). ¹H NMR (DMSO-d₆, 400 MHz) δ 0.92-0.98 (2H, m), 1.03-1.07 (2H, m), 2.54 (3H, s), 2.97-3.07 (2H, m), 3.14-3.24 (2H, m), 3.50-3.64 (3H, m), 3.86 (3H, s), 4.08 (2H, t), 4.25 (2H, t), 6.47 (2H, s), 6.78-6.80 (1H, m), 6.87-6.91 (1H, m), 7.03-7.17 (2H, m), 7.47 (1H, d), 7.85 (2H, d).

Step 7. Preparation of 2-[5-(2-{[2-(2-amino-1,3-benzodiazol-1-yl)ethyl](2,2,2-trifluoroethyl)amino}ethoxy)-1-cyclopropylpyrazol-4-yl]-6-methylpyridine-4-carboxylic acid (INT-B6-7). A mixture of methyl 2-[5-(2-{[2-(2-amino-1,3-benzodiazol-1-yl)ethyl](2,2,2-trifluoroethyl)amino}ethoxy)-1-cyclopropylpyrazol-4-yl]-6-methylpyridine-4-carboxylate (120 mg, 0.22 mmol, 1 equiv) and LiOH (10 mg, 0.43 mmol, 2 equiv) in tetrahydrofuran (4 mL) and H₂O (1 mL) was stirred for 1 h at room temperature under nitrogen atmosphere. The mixture was concentrated in vacuo. The residue was purified by reverse flash chromatography using water and acetonitrile as mobile phase. Fractions containing the desired compound were evaporated to dryness to give 90 mg of 2-[5-(2-{[2-(2-amino-1,3-benzodiazol-1-yl)ethyl](2,2,2-trifluoroethyl)amino}ethoxy)-1-cyclopropylpyrazol-4-yl]-6-methylpyridine-4-carboxylic acid (INT-B6-7) as a brown solid (76%). LCMS: m/z (EST), [M+H]⁺=544.30.

Step 8. Preparation of 5-cyclopropyl-26-methyl-10-(2,2,2-trifluoroethyl)-7-oxa-4,5,10,13,20,22,27-heptaazapentacyclo[22.3.1.0\hat{ }{2,6}.0\hat{ }{13,21}.0\hat{ }{14,19}]octacosa-1(27),2(6),3,14,16,18,20,24(28),25-nonaen-23-one (EXAMPLE B6). To a stirred mixture of 2-[5-(2-{[2-(2-amino-1,3-benzodiazol-1-yl)ethyl](2,2,2-trifluoroethyl)amino}ethoxy)-1-cyclopropylpyrazol-4-yl]-6-methylpyridine-4-carboxylic acid (80 mg, 0.14 mmol, 1 equiv) and N,N,N,N-tetramethyl-O-(7-azabenzotriazol-1-yl)uronium hexafluorophospate (83 mg, 0.22 mmol, 1.5 equiv) and 1,4-dioxane (3 mL) was added N,N-diisopropylethylamine (57 mg, 0.44 mmol, 3.0 equiv) at room temperature under nitrogen atmosphere. The reaction mixture was stirred for 2 h at that temperature and concentrated in vacuo. The residue was purified by Prep-HPLC using water containing 10 mmol/L NH₄HCO₃ and 0.1% NH₄OH and acetonitrile as mobile phase to give 40 mg of 5-cyclopropyl-26-methyl-10-(2,2,2-trifluoroethyl)-7-oxa-4,5,10,13,20,22,27-heptaazapentacyclo[22.3.1.0\hat{ }{2,6}.0\hat{ }{13,21}.0\hat{ }{14,19}]octacosa-1(27),2(6),3,14,16,18,20,24(28),25-nonaen-23-one (EXAMPLE B6) as a white solid (52%). LCMS: m/z (EST), [M+H]⁺=526.20. ¹H NMR (DMSO-d₆, 400 MHz) δ 0.98-1.08 (2H, m), 1.04-1.15 (2H, m), 2.48-2.54 (1H, m), 2.57 (3H, s), 3.14-3.23 (2H, m), 3.33-3.38 (1H, m), 3.56-3.73 (3H, m), 4.31 (2H, t), 4.34-4.42 (2H, m), 7.20-7.35 (2H, m), 7.51-7.59 (2H, m), 7.72-7.79 (1H, m), 7.85 (1H, s), 8.55 (1H, d), 12.73 (1H, s).

### Example B7A and Example B7B

### 15,21-Dimethyl-23,29-dioxa-2,9,11,16,20,21,26-heptaazahexacyclo[24.4.1.1\hat{ }{13,17}.0\hat{ }{2,10}.0\hat{ }{3,8}.0\hat{ }{18,22}1]dotriaconta-3,5,7,9,13(32),14,16,18(22),19-nonaen-12-one, Isomer 1 (Example B7A) and Isomer 2 (Example B7B)

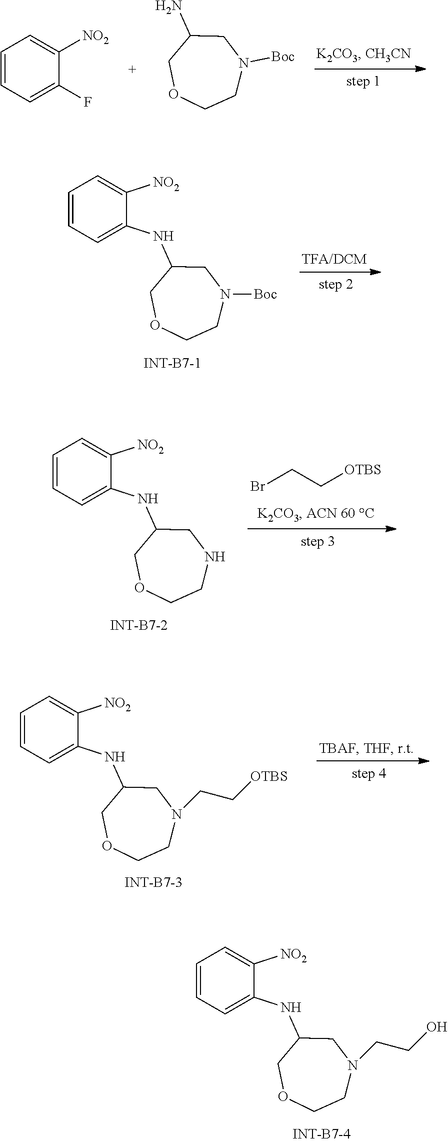

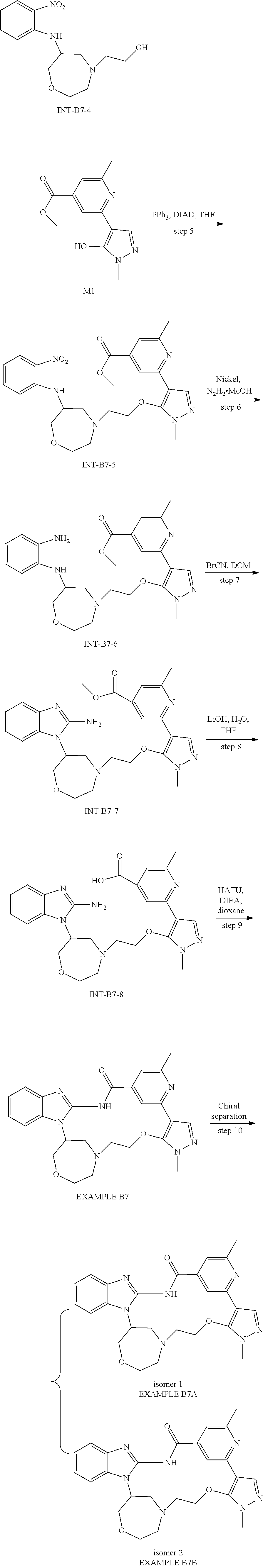

Step 1. Preparation of tert-butyl 6-[(2-nitrophenyl)amino]-1,4-oxazepane-4-carboxylate (INT-B7-1). A mixture of O-fluoronitrobenzene (1 g, 7.09 mmol, 1 equiv), K₂CO₃ (1.96 g, 14.17 mmol, 2 equiv), and tert-butyl 6-amino-1,4-oxazepane-4-carboxylate (1.69 g, 7.79 mmol, 1.1 equiv) in acetonitrile (20 mL) was stirred for 12 h at 100 °C under nitrogen atmosphere. The mixture was cooled to room temperature. The reaction was quenched with water (200 mL) and the mixture was extracted with ethyl acetate (3×200 mL). The combined organic layers were washed with brine (3×100 mL), dried over anhydrous Na₂SO₄, and concentrated under reduced pressure. The residue was purified on a silica gel column eluted with petroleum ether/ethyl acetate (3/1) to give 1.9 g of tert-butyl 6-[(2-nitrophenyl)amino]-1,4-oxazepane-4-carboxylate (INT-B7-1) as a yellow oil (79%). LCMS: m/z (ESI), [M+H]⁺=338.10. ¹H NMR (DMSO-d₆, 400 MHz) δ 1.03 (6H, s), 1.33 (3H, s), 3.29 (2H, d), 3.58-3.79 (2H, m), 3.81-3.96 (2H, m), 4.02-4.13 (2H, m), 6.72 (1H, q), 7.17 (1H, d), 7.55 (1H, q), 8.05-8.13 (1H, m), 8.36-8.46 (1H, m).

Step 2. Preparation of N-(2-nitrophenyl)-1,4-oxazepan-6-amine (INT-B7-2). A mixture of tert-butyl 6-[(2-nitrophenyl)amino]-1,4-oxazepane-4-carboxylate (2 g, 5.928 mmol, 1 equiv) in trifluoroacetic acid (10 mL) and dichloromethane (40 mL) was stirred overnight at room temperature under nitrogen atmosphere. The mixture was concentrated under reduced pressure. The residue was treated with saturated NaHCO₃ (aq.) (100 mL) and extracted with ethyl acetate (3×100 mL). The combined organic layers were washed with brine (3×200 mL), dried over anhydrous Na₂SO₄, and concentrated under reduced pressure to give 1.4 g of N-(2-nitrophenyl)-1,4-oxazepan-6-amine (INT-B7-2) as a yellow oil (99%). This material was used for the next reaction without further purification. LCMS: m/z (ESI), [M+H]⁺=238.10.

Step 3. Preparation of 4-{2-[(tert-butyldimethylsilyl)oxy]ethyl}-N-(2-nitrophenyl)-1,4-oxazepan-6-amine (INT-B7-3). A mixture of N-(2-nitrophenyl)-1,4-oxazepan-6-amine (1.4 g, 5.90 mmol, 1 equiv), (2-bromoethoxy)(tert-butyl)dimethylsilane (2.12 g, 8.85 mmol, 1.5 equiv), and K₂CO₃ (2.45 g, 17.70 mmol, 3 equiv) in acetonitrile (20 mL) was stirred overnight at 60 °C under nitrogen atmosphere and cooled to room temperature. The reaction was quenched with water (100 mL) and the mixture was extracted with ethyl acetate (3×100 mL). The combined organic layers were washed with brine (3×200 mL), dried over anhydrous Na₂SO₄, and concentrated under reduced pressure. The residue was purified on a silica gel column eluted with petroleum ether/ethyl acetate (1/1) to give 1.8 g of 4-{2-[(tert-butyldimethylsilyl)oxy]ethyl}-N-(2-nitrophenyl)-1,4-oxazepan-6-amine (INT-B7-3) as an orange oil (77%). LCMS: m/z (EST), [M+H]⁺=396.15.

Step 4. Preparation of 2-{6-[(2-nitrophenyl)amino]-1,4-oxazepan-4-yl}ethanol (INT-B7-4). A mixture of 4-{2-[(tert-butyldimethylsilyl)oxy]ethyl}-N-(2-nitrophenyl)-1,4-oxazepan-6-amine (1.7 g, 4.29 mmol, 1 equiv) and tetrabutylammonium fluoride (2.25 g, 8.59 mmol, 2 equiv) in tetrahydrofuran (20 mL) was stirred for 2 h at room temperature under nitrogen atmosphere. The reaction was quenched with water (100 mL) and the mixture was extracted with ethyl acetate (3×100 mL). The combined organic layers were washed with brine (3×200 mL), dried over anhydrous Na₂SO₄, and concentrated under reduced pressure. The residue was purified by C18 FLASH using water containing 10 mmol/L NH₄HCO₃ and 0.1% NH₃·H₂O and acetonitrile as mobile phase. Fractions containing the desired compound were evaporated to dryness to give 1 g of 2-{6-[(2-nitrophenyl)amino]-1,4-oxazepan-4-yl}ethanol (INT-B7-4) as an orange oil (82%). LCMS: m/z (EST), [M+H]+=282.10. ¹H NMR (DMSO-dd, 400 MHz) δ 2.64-2.71 (3H, m), 2.81-2.94 (2H, m), 3.03-3.18 (1H, m), 3.45-3.59 (2H, m), 3.59-3.75 (3H, m), 3.95-3.99 (1H, m), 4.04-4.13 (1H, m), 4.41 (1H, t), 6.66-7.00 (1H, m), 7.01-7.02 (1H, m), 7.49-7.53 (1H, m), 8.07-8.10 (1H, m), 8.79 (1H, d)

Step 5. Preparation of methyl 2-methyl-6-[1-methyl-5-(2-{6-[(2-nitrophenyl)amino]-1,4-oxazepan-4-yl}ethoxy)pyrazol-4-yl]pyridine-4-carboxylate (INT-B7-5). To a stirred mixture of 2-{6-[(2-nitrophenyl)amino]-1,4-oxazepan-4-yl}ethanol (1.00 g, 3.55 mmol, 1.2 equiv) and triphenylphosphine (2.33 g, 8.89 mmol, 3 equiv) in tetrahydrofuran (20 mL) were added diisopropyl azodicarboxylate (1.80 g, 8.89 mmol, 3 equiv) and methyl 2-(5-hydroxy-1-methylpyrazol-4-yl)-6-methylpyridine-4-carboxylate (0.73 g, 2.96 mmol, 1.00 equiv) in THF (10 mL) at 0 °C under nitrogen atmosphere. The mixture was stirred at 0 °C for 2 h. The reaction was quenched with water (100 mL) and the mixture was extracted with ethyl acetate (3×100 mL). The combined organic layers were washed with brine (3×200 mL), dried over anhydrous Na₂SO₄ and concentrated under reduced pressure. The residue was purified on a silica gel column eluted with petroleum ether/ethyl acetate (1/1) to give 0.9 g of methyl 2-methyl-6-[1-methyl-5-(2-{6-[(2-nitrophenyl)amino]-1,4-oxazepan-4-yl}ethoxy)pyrazol-4-yl]pyridine-4-carboxylate (INT-B7-5) as an orange oil (59%). LCMS: m/z (ESI), [M+H]⁺=511.20. ¹H NMR (DMSO-d₆, 400 MHz) δ 2.52 (3H, s), 2.91-2.95 (2H, m), 3.05 (1H, d), 3.10 (1H, q), 3.17 (2H, d), 3.67 (2H, d), 3.70 (3H, s), 3.87 (3H, s), 3.90-3.95 (1H, m), 4.05-4.12 (2H, m), 4.20-4.34 (2H, m), 6.64-6.68 (1H, m), 6.97 (1H, d), 7.42 (1H, d), 7.45-7.50 (1H, m), 7.81 (1H, d), 7.88 (1H, s), 8.02-8.05 (1H, m), 8.67 (1H, d).

Step 6. Preparation of methyl 2-[5-(2-{6-[(2-aminophenyl)amino]-1,4-oxazepan-4-yl}ethoxy)-1-methylpyrazol-4-yl]-6-methylpyridine-4-carboxylate (INT-B7-6). To a stirred mixture of methyl 2-methyl-6-[1-methyl-5-(2-{6-[(2-nitrophenyl)amino]-1,4-oxazepan-4-yl}ethoxy)pyrazol-4-yl]pyridine-4-carboxylate (540 mg, 1.06 mmol, 1 equiv) and Raney Nickel (725 mg, 8.46 mmol, 8 equiv) in methanol (5 mL) was added hydrazine hydrate (79 mg, 1.59 mmol, 1.5 equiv) at 0 °C. The mixture was stirred for 2 h at 0 °C under nitrogen atmosphere. The resulting mixture was filtered. The filter cake was washed with methanol (3×50 mL) and the resulting solution was concentrated under reduced pressure to give 480 mg of methyl 2-[5-(2-{6-[(2-aminophenyl)amino]-1,4-oxazepan-4-yl}ethoxy)-1-methylpyrazol-4-yl]-6-methylpyridine-4-carboxylate (INT-B7-6) as a brown solid. This material was used for the next reaction without further purification. LCMS: m/z (EST), [M+H]⁺=481.25.

Step 7. Preparation of methyl 2-(5-{2-[6-(2-amino-1,3-benzodiazol-1-yl)-1,4-oxazepan-4-yl]ethoxy}-1-methylpyrazol-4-yl)-6-methylpyridine-4-carboxylate (INT-B7-7).

A mixture of methyl 2-[5-(2-{6-[(2-aminophenyl)amino]-1,4-oxazepan-4-yl}ethoxy)-1-methylpyrazol-4-yl]-6-methylpyridine-4-carboxylate (500 mg, 1.04 mmol, 1 equiv) and BrCN (132 mg, 1.25 mmol, 1.2 equiv) in dichloromethane (5 mL) was stirred overnight at room temperature under nitrogen atmosphere. The reaction was quenched with water (80 mL) and the mixture was extracted with ethyl acetate (3×60 mL). The combined organic layers were washed with brine (3×50 mL), dried over anhydrous Na₂SO₄, and concentrated under reduced pressure. The residue was purified by Prep-TLC eluted with petroleum ether/ethyl acetate (1/1) to give 400 mg of methyl 2-(5-{2-[6-(2-amino-1,3-benzodiazol-1-yl)-1,4-oxazepan-4-yl]ethoxy}-1-methylpyrazol-4-yl)-6-methylpyridine-4-carboxylate (INT-B7-7) as an orange oil (76%). LCMS: m/z (EST), [M+H]⁺=506.30.

Step 8. Preparation of 2-(5-{2-[6-(2-amino-1,3-benzodiazol-1-yl)-1,4-oxazepan-4-yl]ethoxy}-1-methylpyrazol-4-yl)-6-methylpyridine-4-carboxylic acid (INT-B7-8). A mixture of methyl 2-(5-{2-[6-(2-amino-1,3-benzodiazol-1-yl)-1,4-oxazepan-4-yl]ethoxy}-1-methylpyrazol-4-yl)-6-methylpyridine-4-carboxylate (200 mg, 0.39 mmol, 1 equiv) and LiOH·H₂O (25 mg, 0.59 mmol, 1.5 equiv) in tetrahydrofuran (4 mL) and water (1 mL) was stirred for 2 h at room temperature under nitrogen atmosphere and concentrated under reduced pressure. The residue was purified by C18 FLASH using water containing 10 mmol/L NH₄HCO₃ and 0.1% NH₃—H₂O and acetonitrile as mobile phase. Fractions containing the desired compound were evaporated to dryness to give 180 mg of 2-(5-{2-[6-(2-amino-1,3-benzodiazol-1-yl)-1,4-oxazepan-4-yl]ethoxy}-1-methylpyrazol-4-yl)-6-methylpyridine-4-carboxylic acid (INT-B7-8) as a pink solid (92%). LCMS: m/z (ESI), [M+H]⁺=492.30.

Step 9. Preparation of 15,21-dimethyl-23,29-dioxa-2,9,11,16,20,21,26-heptaazahexacyclo[24.4.1.1\hat{ }{13,17}.0\hat{ }{2,10}.0\hat{ }{3,8}.0\hat{ }{18,22}]dotriaconta-3,5,7,9,13(32),14,16,18(22),19-nonaen-12-one (EXAMPLE B7). A mixture of 2-(5-{2-[6-(2-amino-1,3-benzodiazol-1-yl)-1,4-oxazepan-4-yl]ethoxy}-1-methylpyrazol-4-yl)-6-methylpyridine-4-carboxylic acid (160 mg, 0.33 mmol, 1 equiv), HATU (186 mg, 0.49 mmol, 1.5 equiv) and N,N-diisopropylethylamine (126 mg, 0.98 mmol, 3 equiv) in 1,4-dioxane (5 mL) was stirred overnight at room temperature under nitrogen atmosphere. The reaction was quenched with water (30 mL) and the mixture was extracted with ethyl acetate (3×20 mL). The combined organic layers were washed with brine (3×30 mL), dried over anhydrous Na₂SO₄, and concentrated under reduced pressure. The residue was purified by trituration with methanol (50 mL) to give 8.3 mg of 15,21-dimethyl-23,29-dioxa-2,9,11,16,20,21,26-heptaazahexacyclo[24.4.1.1\hat{ }{13,17}.0\hat{ }{2,10}.0\hat{ }{3,8}.0\hat{ }{18,22}]dotriaconta-3,5,7,9,13(32),14,16,18(22),19-nonaen-12-one (EXAMPLE B7) as a white solid (5%). LCMS: m/z (ESI), [M+H]⁺=474.15. ¹H NMR (DMSO-d₆, 400 MHz) δ 2.57 (3H, s), 2.82-2.91 (3H, m), 2.93-3.01 (1H, m), 3.24-3.44 (2H, m), 3.75 (3H, s), 3.72-3.78 (1H, m), 3.96 (1H, br s), 4.03-4.26 (2H, m), 4.59 (2H, d), 5.45 (1H, br s), 7.20-7.33 (2H, m), 7.52-7.58 (2H, m), 7.69 (1H, d), 7.92 (1H, s), 8.61 (1H, s), 12.67 (1H, s).

Step 10. Preparation of 15,21-dimethyl-23,29-dioxa-2,9,11,16,20,21,26-heptaazahexacyclo[24.4.1.1\hat{ }{13,17}.0\hat{ }{2,10}.0\hat{ }{3,8}.0\hat{ }{18,22}]dotriaconta-3,5,7,9,13(32),14,16,18(22),19-nonaen-12-one, isomer1 (EXAMPLE B7A) and isomer 2 (EXAMPLE B7B). The mixture of 15,21-dimethyl-23,29-dioxa-2,9,11,16,20,21,26-heptaazahexacyclo[24.4.1.1\hat{ }{13,17}.0\hat{ }{2,10}.0\hat{ }{3,8}.0\hat{ }{18,22}]dotriaconta-3,5,7,9,13(32),14,16,18(22),19-nonaen-12-one (EXAMPLE B7, 80 mg) was separated by Prep-Chiral-HPLC with CHIRALPAK IG column (2*25 cm, 5 μm) using hexanes containing 0.2% DEA and EtOH/CH₂Cl₂ (1/1) as mobile phase to afford 32 mg of isomer 1 (EXAMPLE B7A, 40%) and 34 mg of isomer 2 (EXAMPLE B7B, 42%) as a white solid.

Isomer 1: LCMS: m/z (ESI), [M+H]⁺=474.15. ¹H NMR (DMSO-d₆, 400 MHz) δ 2.57 (3H, s), 2.82-2.91 (3H, m), 2.93-3.01 (1H, m), 3.24-3.44 (2H, m), 3.75 (3H, s), 3.72-3.78 (1H, m), 3.96 (1H, br s), 4.03-4.26 (2H, m), 4.59 (2H, d), 5.45 (1H, br s), 7.20-7.33 (2H, m), 7.52-7.58 (2H, m), 7.69 (1H, d), 7.92 (1H, s), 8.61 (1H, s), 12.67 (1H, s). Chiral-HPLC, Rt=2.016 min.

Isomer 2: LCMS: m/z (ESI), [M+H]⁺=474.15. ¹H NMR (DMSO-d₆, 400 MHz) δ 2.57 (3H, s), 2.82-2.91 (3H, m), 2.93-3.01 (1H, m), 3.24-3.44 (2H, m), 3.75 (3H, s), 3.72-3.78 (1H, m), 3.96 (1H, br s), 4.03-4.26 (2H, m), 4.59 (2H, d), 5.45 (1H, br s), 7.20-7.33 (2H, m), 7.52-7.58 (2H, m), 7.69 (1H, d), 7.92 (1H, s), 8.61 (1H, s), 12.67 (1H, s). Chiral-HPLC, Rt=2.626 min.

### Example B8A and Example B8B

### 15,21-dimethyl-23-oxa-2,9,11,16,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,15,17(33),18(22),19-nonaen-12-one, Isomer 1 (Example B8A) and Isomer 2 (Example B8B)

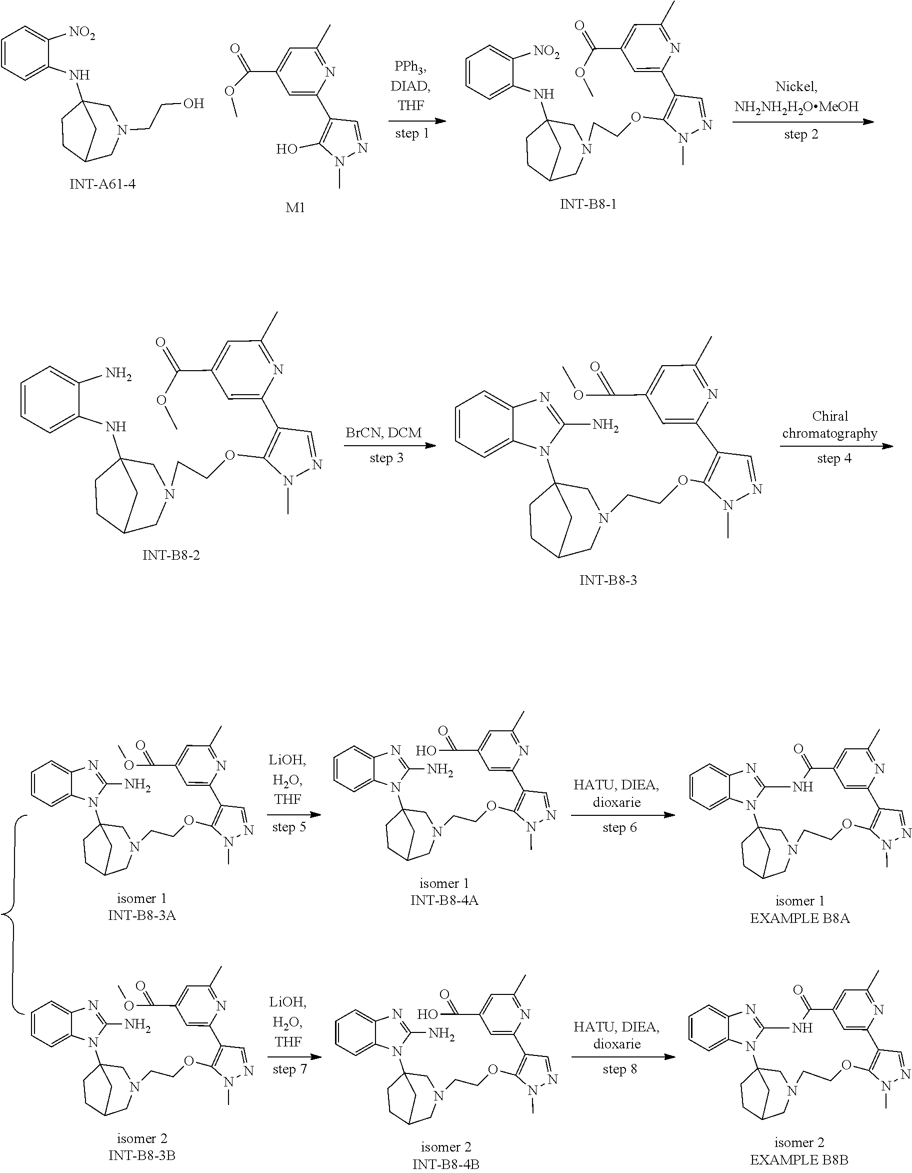

Step 1. Preparation of methyl 2-methyl-6-[1-methyl-5-(2-{1-[(2-nitrophenyl)amino]-3-azabicyclo[3.2.1]octan-3-yl}ethoxy)pyrazol-4-yl]pyridine-4-carboxylate (INT-B8-1). To a stirred mixture of 2-{1-[(2-nitrophenyl)amino]-3-azabicyclo[3.2.11]octan-3-yl}ethanol (INT-A61-4, 380 mg, 1.30 mmol, 1 equiv) and triphenylphosphine (685 mg, 2.61 mmol, 2 equiv) in tetrahydrofuran (10 mL) were added methyl 2-(5-hydroxy-1-methylpyrazol-4-yl)-6-methylpyridine-4-carboxylate (M1, 323 mg, 1.30 mmol, 1 equiv) and diisopropyl azodicarboxylate (528 mg, 2.61 mmol, 2 equiv) at 0 °C under nitrogen atmosphere. The resulting mixture was stirred for 2 h at 0 °C. The reaction was quenched with water (30 mL) and the mixture was extracted with CH₂Cl₂ (3×50 mL). The combined organic layers were washed with brine (3×75 mL), dried over anhydrous Na₂SO₄, and concentrated under reduced pressure. The residue was purified by prep-TLC eluted with petroleum ether/ethyl acetate (1/1) to give 460 mg of methyl 2-methyl-6-[1-methyl-5-(2-{1-[(2-nitrophenyl)amino]-3-azabicyclo[3.2.1]octan-3-yl}ethoxy)pyrazol-4-yl]pyridine-4-carboxylate (INT-B8-1) as a white solid (67%). LCMS: m/z (EST), [M+H]⁺=521.15.

Step 2. Preparation of methyl 2-[5-(2-{1-[(2-aminophenyl)amino]-3-azabicyclo[3.2.1]octan-3-yl}ethoxy)-1-methylpyrazol-4-yl]-6-methylpyridine-4-carboxylate (INT-B8-2). To a stirred mixture of methyl 2-methyl-6-[1-methyl-5-(2-{1-[(2-nitrophenyl)amino]-3-azabicyclo[3.2.1]octan-3-yl}ethoxy)pyrazol-4-yl]pyridine-4-carboxylate (500 mg, 0.96 mmol, 1 equiv) and Raney-Ni (80 mg) in methanol (10 mL) was added hydrazine hydrate (92 mg, 2.88 mmol, 3 equiv) at room temperature under nitrogen atmosphere. The resulting mixture was stirred for 1 h at room temperature. The resulting mixture was filtered and the filter cake was washed with methanol (3×20 mL). The resulting solution was concentrated under reduced pressure. The mixture was purified by prep-TLC eluted with dichloromethane/methanol (20/1) to give 400 mg of methyl 2-[5-(2-{1-[(2-aminophenyl)amino]-3-azabicyclo[3.2.1]octan-3-yl}ethoxy)-1-methylpyrazol-4-yl]-6-methylpyridine-4-carboxylate (INT-B8-2) as a yellow solid (85%). LCMS: m/z (ESI), [M+H]⁺=491.15.

Step 3. Preparation of methyl 2-(5-{2-[1-(2-amino-1,3-benzodiazol-1-yl)-3-azabicyclo[3.2.1]octan-3-yl]ethoxy}-1-methylpyrazol-4-yl)-6-methylpyridine-4-carboxylate (INT-B8-3). To a stirred mixture of methyl 2-[5-(2-{1-[(2-aminophenyl)amino]-3-azabicyclo[3.2.1]octan-3-yl}ethoxy)-1-methylpyrazol-4-yl]-6-methylpyridine-4-carboxylate (455 mg, 0.93 mmol, 1 equiv) in ethanol (20 mL) was added BrCN (334 mg, 3.15 mmol, 3.4 equiv) at room temperature under nitrogen atmosphere. The resulting mixture was stirred for 1 h at room temperature and concentrated under reduced pressure. The residue was purified by prep-TLC eluted with dichloromethane/methanol (20/1) to give 350 mg of methyl 2-(5-{2-[1-(2-amino-1,3-benzodiazol-1-yl)-3-azabicyclo[3.2.1]octan-3-yl]ethoxy}-1-methylpyrazol-4-yl)-6-methylpyridine-4-carboxylate (INT-B8-3) as a brown solid (73%). LCMS: m/z (EST), [M+H]⁺=516.15.

Step 4. Preparation of methyl 2-(5-{2-[1-(2-amino-1,3-benzodiazol-1-yl)-3-azabicyclo[3.2.1]octan-3-yl]ethoxy}-1-methylpyrazol-4-yl)-6-methylpyridine-4-carboxylate, isomer 1, (INT-B8-3A) and isomer 2 (INT-B8-3B).

The racemic mixture of methyl 2-(5-{2-[1-(2-amino-1,3-benzodiazol-1-yl)-3-azabicyclo[3.2.1]octan-3-yl]ethoxy}-1-methylpyrazol-4-yl)-6-methylpyridine-4-carboxylate (350 mg) was separated by Prep-Chiral-HPLC with CHIRALPAK IG chiral column (2×25 cm, 5 μm) using methyl tert butyl ether containing 0.10% diethylamine and ethanol/CH₂Cl₂ (1/1) as mobile phase to afford 90 mg of isomer 1 (INT-B8-3A, 25%) and 80 mg isomer 2 (INT-B8-3B, 22%) as white solid. Chiral-HPLC: Rt (isomer 1)=1.312 min, Rt (isomer 2)=2.047 min.

Step 5. Preparation of 2-(5-{2-[1-(2-amino-1,3-benzodiazol-1-yl)-3-azabicyclo[3.2.1]octan-3-yl]ethoxy}-1-methylpyrazol-4-yl)-6-methylpyridine-4-carboxylic acid (INT-B8-4A). To a stirred mixture of methyl 2-(5-{2-[1-(2-amino-1,3-benzodiazol-1-yl)-3-azabicyclo[3.2.1]octan-3-yl]ethoxy}-1-methylpyrazol-4-yl)-6-methylpyridine-4-carboxylate (INT-B8-3A, 85 mg, 0.16 mmol, 1 equiv) in tetrahydrofuran (8 mL) was added LiOH H₂O (16 mg, 0.66 mmol, 4 equiv) in H₂O (2 mL) at room temperature under nitrogen atmosphere. The resulting mixture was stirred for 30 min at 60 °C, cooled to room temperature, and concentrated under reduced pressure. The residue was purified by reverse flash chromatography with C18 silica gel column using water and acetonitrile as mobile phase. Fractions containing the desired compound were evaporated to dryness to give 80 mg of 2-(5-{2-[1-(2-amino-1,3-benzodiazol-1-yl)-3-azabicyclo[3.2.1]octan-3-yl]ethoxy}-1-methylpyrazol-4-yl)-6-methylpyridine-4-carboxylic acid (INT-B8-4A) as a yellow solid (96%). LCMS: m/z (ESI), [M+H]⁺=502.20.

Step 6. Preparation of 15,21-dimethyl-23-oxa-2,9,11,16,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,15,17(33),18(22),19-nonaen-12-one, isomer 1 (EXAMPLE B8A). A mixture of N,N-diisopropylethylamine (58 mg, 0.45 mmol, 3 equiv), 2-(5-{2-[1-(2-amino-1,3-benzodiazol-1-yl)-3-azabicyclo[3.2.1]octan-3-yl]ethoxy}-1-methylpyrazol-4-yl)-6-methylpyridine-4-carboxylic acid (INT-B8-4A, 75 mg, 0.15 mmol, 1 equiv) and N,N,N,N-tetramethyl-O-(7-azabenzotriazol-1-yl)uroniumhexafluorophospate (85 mg, 0.22 mmol, 1.5 equiv) in 1,4-dioxane (7 mL) was stirred for 1 h at 60 °C under nitrogen atmosphere. The mixture was cooled to room temperature. The reaction was quenched with water (20 mL) and the mixture was extracted with CH₂Cl₂ (3×30 mL). The combined organic layers were washed with brine (3×30 mL), dried over anhydrous Na₂SO₄, and concentrated under reduced pressure. The residue was purified by Prep-TLC and eluted with dichloromethane/methanol (25/1) to give 57.4 mg of 15,21-dimethyl-23-oxa-2,9,11,16,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,15,17(33),18(22),19-nonaen-12-one (isomer 1, EXAMPLE B8A) as a yellow solid (79%). LCMS: m/z (EST), [M+H]⁺=484.30. ¹H NMR (DMSO-d₆, 400 MHz) δ 1.59-1.80 (3H, m), 2.02 (1H, d), 2.38-2.50 (3H, m), 2.56 (3H, s), 2.63-2.82 (3H, m), 3.22-3.45 (2H, m), 3.74 (3H, s), 3.77-3.83 (1H, m), 4.56 (1H, t), 4.71 (1H, d), 7.16-7.25 (2H, m), 7.55-7.63 (2H, m), 7.73-7.84 (1H, m), 7.94 (1H, s), 8.44 (1H, s), 12.87 (1H, s). SFC-HPLC, Rt=1.282 min.

Step 7. Preparation of 2-(5-{2-[1-(2-amino-1,3-benzodiazol-1-yl)-3-azabicyclo[3.2.1]octan-3-yl]ethoxy}-1-methylpyrazol-4-yl)-6-methylpyridine-4-carboxylic acid (INT-B8-4B). To a stirred mixture of methyl 2-(5-{2-[1-(2-amino-1,3-benzodiazol-1-yl)-3-azabicyclo[3.2.1]octan-3-yl]ethoxy}-1-methylpyrazol-4-yl)-6-methylpyridine-4-carboxylate (INT-B8-3B, 75 mg, 0.15 mmol, 1 equiv) in tetrahydrofuran (8 mL) was added LiOH H₂O (14 mg, 0.58 mmol, 4 equiv) in H₂O (2 mL) at room temperature under nitrogen atmosphere. The resulting mixture was stirred for 30 min at 60 °C. The mixture was cooled to room temperature and concentrated under reduced pressure. The residue was purified by reverse flash chromatography with C18 silica gel column using water and acetonitrile as mobile phase. Fractions containing the desired compound were evaporated to dryness to give 70 mg of 2-(5-{2-[1-(2-amino-1,3-benzodiazol-1-yl)-3-azabicyclo[3.2.1]octan-3-yl]ethoxy}-1-methylpyrazol-4-yl)-6-methylpyridine-4-carboxylic (INT-B8-4B) acid as an off-white solid (96%). LCMS: m/z (ESI), [M+H]⁺=502.20.

Step 8. Preparation of 15,21-dimethyl-23-oxa-2,9,11,16,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,15,17(33),18(22),19-nonaen-12-one, isomer 2 (EXAMPLE B8B). A mixture of N,N-diisopropylethylamine (50 mg, 0.39 mmol, 3 equiv), 2-(5-{2-[1-(2-amino-1,3-benzodiazol-1-yl)-3-azabicyclo[3.2.1]octan-3-yl]ethoxy}-1-methylpyrazol-4-yl)-6-methylpyridine-4-carboxylic acid (INT-B8-4B, 65 mg, 0.13 mmol, 1 equiv) and N,N,N,N-tetramethyl-O-(7-azabenzotriazol-1-yl)uroniumhexafluorophospate (74 mg, 0.20 mmol, 1.5 equiv) in 1,4-dioxane (6 mL) was stirred for 1 h at 60 °C. The mixture was cooled to room temperature. The reaction was quenched with water (20 mL) and the mixture was extracted with CH₂Cl₂ (3×30 mL). The combined organic layers were washed with brine (3×30 mL), dried over anhydrous Na₂SO₄, and concentrated under reduced pressure. The residue was purified by Prep-TLC and eluted with dichloromethane/methanol (25/1) to give 46.5 mg of 15,21-dimethyl-23-oxa-2,9,11,16,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,15,17(33),18(22),19-nonaen-12-one, isomer 2 (EXAMPLE B8B) as a yellow solid (74%). LCMS: m/z (EST), [M+H]⁺=484.25. ¹H NMR (DMSO-d₆, 400 MHz) δ 1.59-1.80 (3H, m), 2.02 (1H, d), 2.38-2.50 (3H, m), 2.56 (3H, s), 2.63-2.82 (3H, m), 3.22-3.45 (2H, m), 3.74 (3H, s), 3.77-3.83 (1H, m), 4.56 (1H, t), 4.71 (1H, d), 7.16-7.25 (2H, m), 7.55-7.63 (2H, m), 7.73-7.84 (1H, m), 7.94 (1H, s), 8.44 (1H, s), 12.87 (1H, s). SFC-HPLC, Rt=1.060 min.

### Example B9A and Example B9B

### 15,21-Dimethyl-5-(4-methylpiperazin-1-yl)-23-oxa-2,9,11,16,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,15,17(33),18(22),19-nonaen-12-one, Isomer 1 (Example B9A) and Isomer 2 (Example B9B)

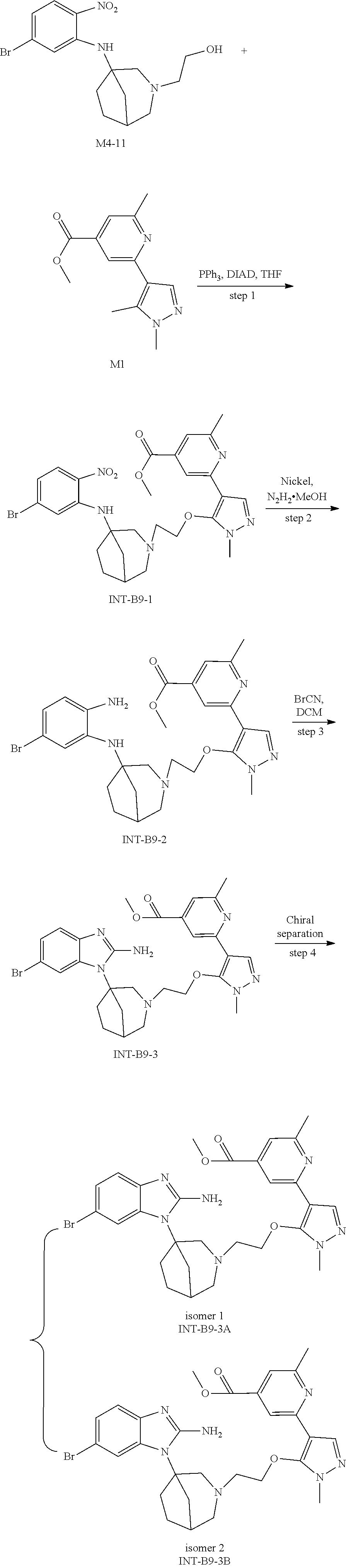

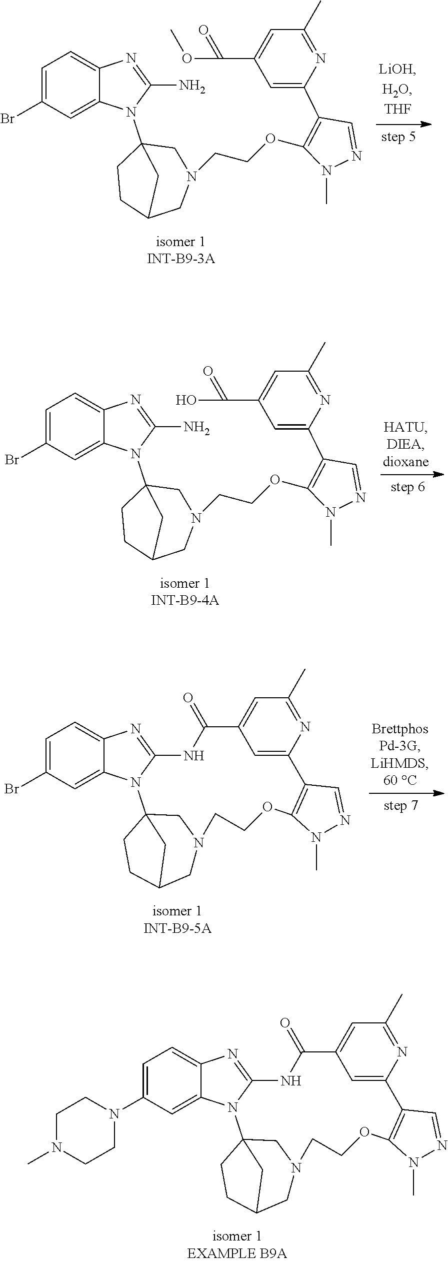 [SMILES: COC(=O)c1cc(C)nc(-c2cnn(C)c2OCCN2CC3CCC(n4c(N)nc5ccc(Br)cc54)(C3)C2)c1 ; Cc1cc(C(=O)O)cc(-c2cnn(C)c2OCCN2CC3CCC(n4c(N)nc5ccc(Br)cc54)(C3)C2)n1 ; Cc1cc2cc(n1)-c1cnn(C)c1OCCN1CC3CCC(C3)(C1)n1c(nc3ccc(Br)cc31)NC2=O ; Cc1cc2cc(n1)-c1cnn(C)c1OCCN1CC3CCC(C3)(C1)n1c(nc3ccc(N4CCN(C)CC4)cc31)NC2=O]

Step 1. Preparation of methyl 2-[5-(2-{1-[(5-bromo-2-nitrophenyl)amino]-3-azabicyclo[3.2.1]octan-3-yl}ethoxy)-1-methylpyrazol-4-yl]-6-methylpyridine-4-carboxylate (INT-B9-1). To a stirred mixture of methyl 2-(5-hydroxy-1-methylpyrazol-4-yl)-6-methylpyridine-4-carboxylate (M1, 1.40 g, 5.67 mmol, 1.05 equiv) and triphenylphosphine (3.97 g, 15.12 mmol, 2.8 equiv) in tetrahydrofuran (30 mL) was added diisopropyl azodicarboxylate (2.73 g, 13.50 mmol, 2.5 equiv) followed by the addition of 2-{1-[(5-bromo-2-nitrophenyl)amino]-3-azabicyclo[3.2.1]octan-3-yl}ethanol (M4-11, 2 g, 5.40 mmol, 1 equiv) at 0 °C under nitrogen atmosphere. After being stirred at 0 °C for 3 h, the reaction was quenched with water (400 mL) and the mixture was extracted with ethyl acetate (3×300 mL). The combined organic layers were washed with brine (3×300 mL), dried over anhydrous Na₂SO₄, and concentrated under reduced pressure. The residue was purified by a silica gel column eluted with dichloromethane/methanol (30/1) to give 2 g of methyl 2-[5-(2-{1-[(5-bromo-2-nitrophenyl)amino]-3-azabicyclo[3.2.1]octan-3-yl}ethoxy)-1-methylpyrazol-4-yl]-6-methylpyridine-4-carboxylate (INT-B9-1) as a yellow oil (61%). LCMS: m/z (EST), [M+H]⁺=601.00.

Step 2. Preparation of methyl 2-[5-(2-{1-[(2-amino-5-bromophenyl)amino]-3-azabicyclo[3.2.1]octan-3-yl}ethoxy)-1-methylpyrazol-4-yl]-6-methylpyridine-4-carboxylate (INT-B9-2). To a stirred mixture of methyl 2-[5-(2-{1-[(5-bromo-2-nitrophenyl)amino]-3-azabicyclo[3.2.1]octan-3-yl}ethoxy)-1-methylpyrazol-4-yl]-6-methylpyridine-4-carboxylate (2 g, 3.33 mmol, 1 equiv) in methanol (60 mL) were added Raney Ni (457 mg, 5.33 mmol, 1.6 equiv) and NH₂NH₂H₂O (835 mg, 16.68 mmol, 5 equiv) at room temperature. The resulting mixture was stirred for 1 h at room temperature. The reaction was quenched with water (300 mL) and the mixture was extracted with ethyl acetate (3×200 mL). The combined organic layers were washed with brine (3×200 mL), dried over anhydrous Na₂SO₄, and concentrated under reduced pressure. The residue was purified by silica gel chromatography eluted with dichloromethane/methanol (20/1) to give 1.34 g of methyl 2-[5-(2-{1-[(2-amino-5-bromophenyl)amino]-3-azabicyclo[3.2.1]octan-3-yl}ethoxy)-1-methylpyrazol-4-yl]-6-methylpyridine-4-carboxylate (INT-B9-2) as a reddish solid (70%). LCMS: m/z (EST), [M+H]⁺=571.05.

Step 3. Preparation of methyl 2-(5-{2-[1-(2-amino-6-bromo-1,3-benzodiazol-1-yl)-3-azabicyclo[3.2.1]octan-3-yl]ethoxy}-1-methylpyrazol-4-yl)-6-methylpyridine-4-carboxylate (INT-B9-3). To a stirred mixture of methyl 2-[5-(2-{1-[(2-amino-5-bromophenyl)amino]-3-azabicyclo[3.2.1]octan-3-yl}ethoxy)-1-methylpyrazol-4-yl]-6-methylpyridine-4-carboxylate (500 mg, 0.87 mmol, 1 equiv) in ethanol (8 mL) was added BrCN (139 mg, 1.31 mmol, 1.5 equiv) at room temperature. The resulting mixture was stirred for 2 h at at room temperature. The reaction was treated with sat. NaHCO₃ (100 mL) and the mixture was extracted with ethyl acetate (3×100 mL). The combined organic layers were dried over anhydrous Na₂SO₄ and concentrated under reduced pressure. The residue was purified by silica gel chromatography eluted with dichloromethane/methanol (15/1) to give 400 mg of methyl 2-(5-{2-[1-(2-amino-6-bromo-1,3-benzodiazol-1-yl)-3-azabicyclo[3.2.1]octan-3-yl]ethoxy}-1-methylpyrazol-4-yl)-6-methylpyridine-4-carboxylate (INT-B9-3) as a reddish oil (76.63%). LCMS: m/z (ESI), [M+H]⁺=596.10.

Step 4. Preparation of methyl 2-(5-{2-[1-(2-amino-6-bromo-1,3-benzodiazol-1-yl)-3-azabicyclo[3.2.1]octan-3-yl]ethoxy}-1-methylpyrazol-4-yl)-6-methylpyridine-4-carboxylate, isomer 1 (INT-B9-3A) and isomer 2 (INT-B9-3B). The racemic mixture of methyl 2-(5-{2-[1-(2-amino-6-bromo-1,3-benzodiazol-1-yl)-3-azabicyclo[3.2.1]octan-3-yl]ethoxy}-1-methylpyrazol-4-yl)-6-methylpyridine-4-carboxylate (400 mg) was separated by Prep-Chiral-HPLC with CHIRAL ART Cellulose-SB column (5*25 cm, 5 μm) using CO₂ and CH₃OH/CH₂Cl₂ (2/1) as mobile phase to give 150 mg of isomer 1 (INT-B9-3A, 42%) and 150 mg of isomer 2 (INT-B9-3B, 42%) as a white solid. SFC-HPLC, Rt (isomer 1)=1.760 min, Rt (isomer 2)=1.889 min.

Step 5. Preparation of 2-(5-{2-1-(2-amino-6-bromo-1,3-benzodiazol-1-yl)-3-azabicyclo[3.2.1]octan-3-yl]ethoxy}-1-methylpyrazol-4-yl)-6-methylpyridine-4-carboxylic acid (isomer 1, INT-B9-4A). To a stirred solution of methyl 2-(5-{2-[1-(2-amino-6-bromo-1,3-benzodiazol-1-yl)-3-azabicyclo[3.2.1]octan-3-yl]ethoxy}-1-methylpyrazol-4-yl)-6-methylpyridine-4-carboxylate (490 mg, 0.82 mmol, 1 equiv) in THE (8 mL) was added LiOH (78 mg, 3.29 mmol, 4 equiv) in H₂O (2 mL) at room temperature. The resulting mixture was stirred for 3 h at room temperature. The mixture was concentrated under reduced pressure and the residue was purified by reversed-phase flash chromatography eluted with water and acetonitrile as mobile phase to give 410 mg of 2-(5-{2-[1-(2-amino-6-bromo-1,3-benzodiazol-1-yl)-3-azabicyclo[3.2.1]octan-3-yl]ethoxy}-1-methylpyrazol-4-yl)-6-methylpyridine-4-carboxylic acid (isomer 1, INT-B9-4A) as a white solid (85%). LCMS: m/z (ESI), [M+H]⁺=580.05.

Step 6. Preparation of 5-bromo-15,21-dimethyl-23-oxa-2,9,11,16,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,15,17(33),18(22),19-nonaen-12-one (isomer 1, INT-B9-5A). To a stirred solution of 2-(5-{2-[1-(2-amino-6-bromo-1,3-benzodiazol-1-yl)-3-azabicyclo[3.2.1]octan-3-yl]ethoxy}-1-methylpyrazol-4-yl)-6-methylpyridine-4-carboxylic acid (390 mg, 0.67 mmol, 1 equiv) in dioxane (10 mL) were added N,N-Diisopropylethylamine (260 mg, 2.01 mmol, 3 equiv) and N,N,N,N-tetramethyl-O-(7-azabenzotriazol-1-yl)uronium hexafluorophospate (383 mg, 1.01 mmol, 1.5 equiv) at room temperature. The reaction mixture was stirred for 2 h at 50 °C under nitrogen atmosphere. After being cooled to room temperature, the reaction was quenched with water (100 mL) and the mixture was extracted with ethyl acetate (3×100 mL). The combined organic layers were washed with brine (3×100 mL), dried over anhydrous Na₂SO₄, and concentrated under reduced pressure. The crude product was purified by prep-TLC eluted with dichloromethane/methanol (25/1) to give 350 mg of 5-bromo-15,21-dimethyl-23-oxa-2,9,11,16,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,15,17(33),18(22),19-nonaen-12-one (isomer 1, INT-B9-5A) as a light yellow solid (92%). LCMS: m/z (EST), [M+H]⁺=564.05.

Step 7. Preparation of 15,21-dimethyl-5-(4-methylpiperazin-1-yl)-23-oxa-2,9,11,16,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,15,17(33),18(22),19-nonaen-12-one (isomer 1, EXAMPLE B9A). To a stirred mixture of 5-bromo-15,21-dimethyl-23-oxa-2,9,11,16,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,15,17(33),18(22),19-nonaen-12-one (130 mg, 0.23 mmol, 1 equiv), 1-methylpiperazine (34.7 mg, 0.35 mmol, 1.5 equiv), and BrettPhos Pd G₃ (62.8 mg, 0.07 mmol, 0.3 equiv) in dioxane (5 mL) was added LiHMDS (2.3 mL, 2.31 mmol, 10 equiv) dropwise at room temperature under nitrogen atmosphere. The reaction mixture was stirred for 1 h at 60 °C under nitrogen atmosphere. After being cooled to room temperature, the reaction was quenched with sat. NH₄Cl (30 mL) and the mixture was extracted with dichloromethane (3×20 mL). The combined organic layers were washed with brine (3×30 mL), dried over anhydrous Na₂SO₄, and concentrated under reduced pressure. The residue was purified by prep-TLC eluted with dichloromethane/methanol (18/1) to give 41.6 mg of 15,21-dimethyl-5-(4-methylpiperazin-1-yl)-23-oxa-2,9,11,16,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,15,17(33),18(22),19-nonaen-12-one, isomer 1 (EXAMPLE B9A) as a white solid (30%). LCMS: m/z (ESI), [M+H]*=582.40. ¹H NMR (DMSO-d₆, 400 MHz) δ 1.52-1.82 (3H, m), 2.03 (1H, d), 2.24 (3H, s), 2.40-2.60 (8H, m), 2.55 (3H, s), 2.64-2.79 (2H, m), 3.12 (4H, q), 3.20-3.30 (1H, m), 3.37-3.45 (1H, m), 3.74 (3H, s), 3.77 (1H, br s), 4.56 (1H, t), 4.70 (1H, d), 6.93 (1H, dd), 7.19 (1H, d), 7.42 (1H, d), 7.57 (1H, d), 7.94 (1H, s), 8.42 (1H, s), 12.69 (1H, s). SFC-HPLC, Rt=3.968 min.

The same reaction sequence described above for the synthesis of EXAMPLE B9A using INT-B9-3B as starting material afforded 48.4 mg of 15,21-dimethyl-5-(4-methylpiperazin-1-yl)-23-oxa-2,9,11,16,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,15,17(33),18(22),19-nonaen-12-one, isomer 2 (EXAMPLE B9B) as a white solid. LCMS: m/z (EST), [M+H]⁺=582.40. ¹H NMR (DMSO-d₆, 400 MHz) δ 1.52-1.82 (3H, m), 2.03 (1H, d), 2.24 (3H, s), 2.40-2.60 (8H, m), 2.55 (3H, s), 2.64-2.79 (2H, m), 3.12 (4H, q), 3.20-3.30 (1H, m), 3.37-3.45 (1H, m), 3.74 (3H, s), 3.77 (1H, br s), 4.56 (1H, t), 4.70 (1H, d), 6.93 (1H, dd), 7.19 (1H, d), 7.42 (1H, d), 7.57 (1H, d), 7.94 (1H, s), 8.42 (1H, s), 12.69 (1H, s). SFC-HPLC, Rt=3.745 min.

### Example B10A and Example B10B

### 15,21-Dimethyl-5-(morpholin-4-yl)-23-oxa-2,9,11,16,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,15,17(33),18(22),19-nonaen-12-one, Isomer 1 (Example B10A) and isomer 2 (Example B10B)

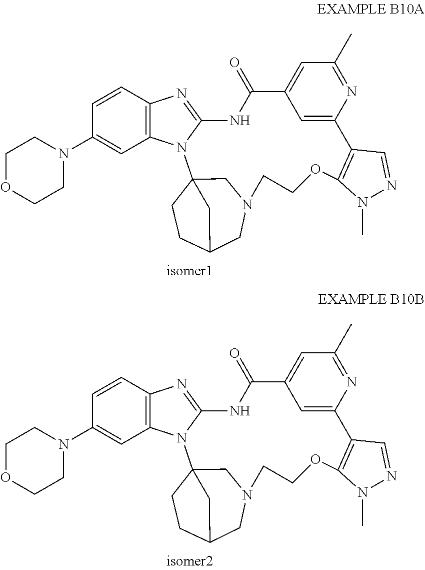 [SMILES: Cc1cc2cc(n1)-c1cnn(C)c1OCCN1CC3CCC(C3)(C1)n1c(nc3ccc(N4CCOCC4)cc31)NC2=O ; Cc1cc2cc(n1)-c1cnn(C)c1OCCN1CC3CCC(C3)(C1)n1c(nc3ccc(N4CCOCC4)cc31)NC2=O]

The title compounds were prepared using the same reaction sequence described for the preparation of EXAMPLE B9A and EXAMPLE-B9B.

EXAMPLE B10A: LCMS: m/z (ESI), [M+H]⁺=569.40. ¹H NMR (DMSO-d₆, 400 MHz) δ 1.56-1.81 (3H, m), 2.03 (1H, d), 2.37-2.48 (3H, m), 2.55 (3H, s), 2.57-2.79 (3H, m), 3.04-3.18 (4H, m), 3.19-3.29 (2H, m), 3.37-3.47 (1H, m), 3.74 (3H, s), 3.75-3.83 (4H, m), 4.56 (11H, t), 4.71 (1H, d), 6.94 (1H, dd), 7.21 (11H, d), 7.45 (11H, d), 7.57 (11H, d), 7.94 (1H, s), 8.43 (1H, s), 12.71 (1H, s). SFC-HPLC, Rt=1.549 min

EXAMPLE B10B: LCMS: m/z (ESI), [M+H]⁺=569.25. ¹H NMR (DMSO-d₆, 400 MHz) δ 1.56-1.81 (3H, m), 2.03 (1H, d), 2.37-2.48 (3H, m), 2.55 (3H, s), 2.57-2.79 (3H, m), 3.04-3.18 (4H, m), 3.19-3.29 (2H, m), 3.37-3.47 (1H, m), 3.74 (3H, s), 3.75-3.83 (4H, m), 4.56 (11H, t), 4.71 (1H, d), 6.94 (1H, dd), 7.21 (11H, d), 7.45 (11H, d), 7.57 (11H, d), 7.94 (1H, s), 8.43 (1H, s), 12.71 (1H, s). SFC-HPLC, Rt=1.639 min

### Example B11

(12R)-5,12,27-Trimethyl-8-oxa-5,14,21,23,28-pentaazapentacyclo[23.3.1.0\hat{ }{2,7}.0\hat{ }{14,22}.0\hat{ }{15,20}]nonacosa-1(29),2,6,15,17,19,21,25,27-nonaene-4,24-dione

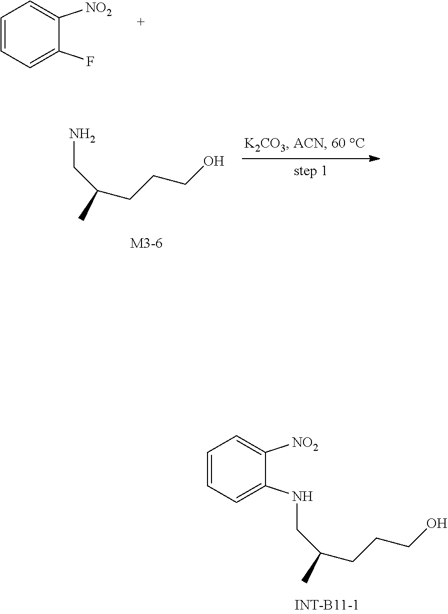

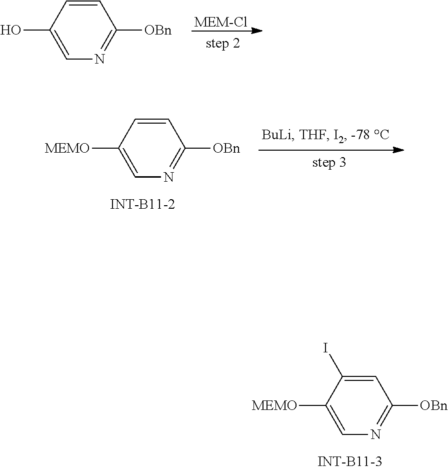

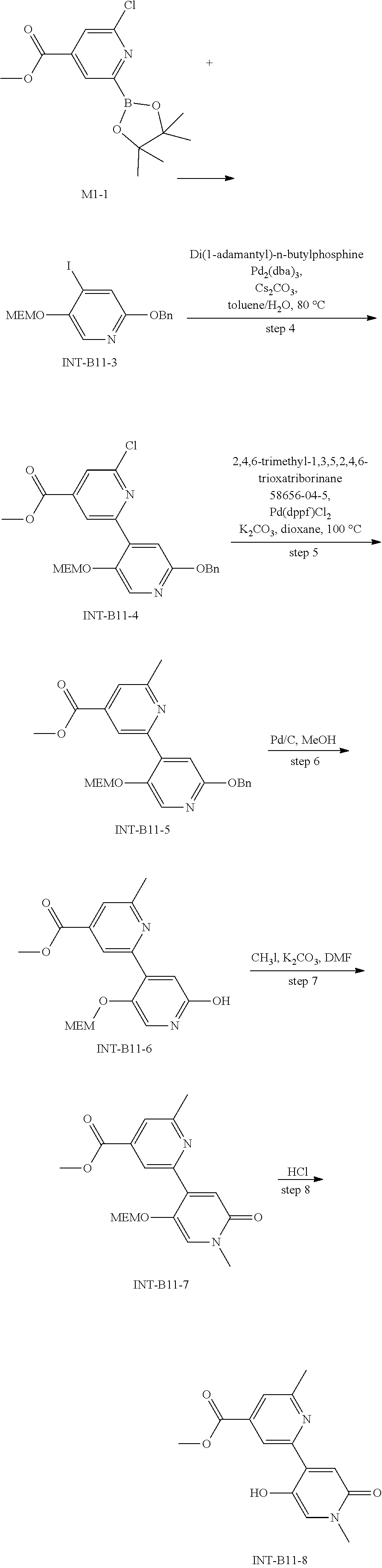

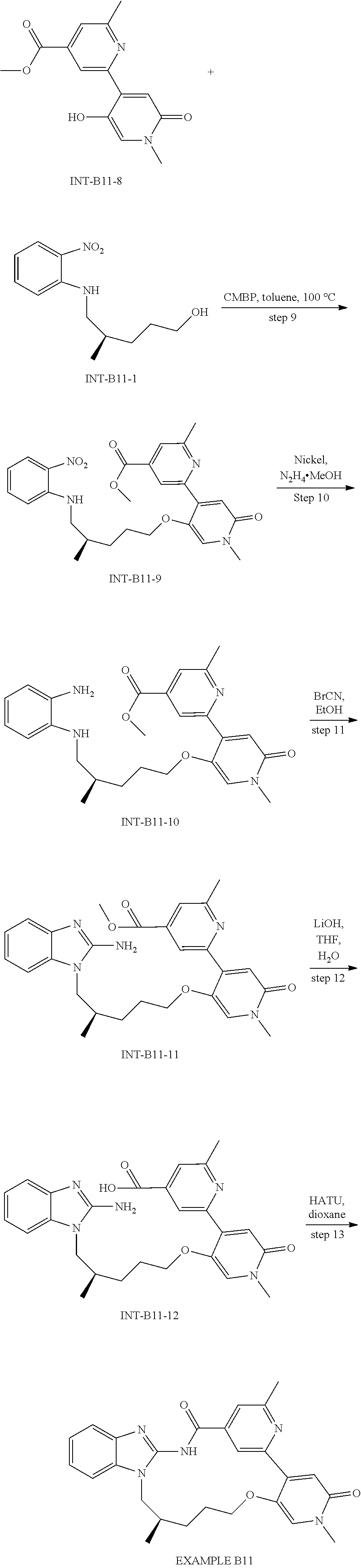

Step 1. Preparation of (4R)-4-methyl-5-[(2-nitrophenyl)amino]pentan-1-ol (INT-B11-1). A mixture of O-fluoronitrobenzene (1.32 g, 9.386 mmol, 1.1 equiv), (4R)-5-amino-4-methylpentan-1-ol (1 g, 8.533 mmol, 1.00 equiv) and K₂CO₃ (3.54 g, 25.599 mmol, 3 equiv) in acetonitrile (20 mL) was stirred for 2 h at 60 °C under nitrogen atmosphere. The mixture was cooled to room temperature. The reaction was quenched with water (800 mL) and the mixture was extracted with dichloromethane (3×600 mL). The combined organic layers were washed with brine (3×500 mL), dried over anhydrous Na₂SO₄, and concentrated under reduced pressure. The residue was purified by silica gel chromatography eluted with petroleum ether/ethyl acetate (1/1) to give 1.6 g of (4R)-4-methyl-5-[(2-nitrophenyl)amino]pentan-1-ol (INT-B11-1) as an orange oil (78%). LCMS: m/z (ESI), [M+H]⁺=239.05.

Step 2. Preparation of 2-(benzyloxy)-5-[(2-methoxyethoxy)methoxy]pyridine (INT-B11-2). To a stirred mixture of 6-(benzyloxy)pyridin-3-ol (5 g, 24.84 mmol, 1 equiv) in N,N-dimethylformamide was added sodium hydride (60% in oil, 4 g, 12.42 mmol, 2 equiv) at 0 °C. The mixture was stirred for 15 min prior to the addition of 1-(chloromethoxy)-2-methoxyethane (3.1 g, 24.84 mmol, 1 equiv). The mixture was stirred for 2 h at 0 °C. The reaction was quenched with water (50 mL) and the mixture was extracted with ethyl acetate (3×50 mL). The combined organic layers were washed with brine (3×50 mL), dried over anhydrous Na₂SO₄, and concentrated under reduced pressure. The residue was purified on a silica gel column eluted with petroleum ether/ethyl acetate (3/1) to give 6 g of 2-(benzyloxy)-5-[(2-methoxyethoxy)methoxy]pyridine (INT-B11-2) as light yellow oil (75%). LCMS: m/z (ESI), [M+H]⁺=290.10. ¹H NMR (DMSO-d₆, 400 MHz) δ 3.22 (3H, s), 3.36-3.51 (2H, m), 3.65-3.80 (2H, m), 5.21 (2H, s), 5.29 (2H, s), 6.85 (1H, d), 7.28-7.42 (3H, m), 7.35-7.50 (2H, m), 7.50 (1H, d), 7.93 (1H, d).

Step 3. Preparation of 2-(benzyloxy)-4-iodo-5-[(2-methoxyethoxy)methoxy]pyridine (INT-B11-3). To a stirred mixture of 2-(benzyloxy)-5-[(2-methoxyethoxy)methoxy]pyridine (5.8 g, 20.04 mmol, 1 equiv) in tetrahydrofuran (5 mL) was added n-butyllithium solution (2.5 M in tetrahydrofuran, 17 mL, 2 mmol) at −78 °C under nitrogen atmosphere. The mixture was stirred at −78 °C for 30 min. Then a solution of I₂ (8.1 g, 32.07 mmol, 1.60 equiv) in 10 mL tetrahydrofuran was added dropwise and the mixture was stirred for 1 h at−78 °C under nitrogen atmosphere. The reaction was quenched with sat. NH₄Cl (100 mL) and the mixture was extracted with ethyl acetate (3×100 mL). The combined organic layers were washed with brine (3×100 mL), dried over anhydrous Na₂SO₄, and concentrated under reduced pressure. The residue was purified on a silica gel column eluted with petroleum ether/ethyl acetate (3/1) to give 5.5 g of 2-(benzyloxy)-4-iodo-5-[(2-methoxyethoxy)methoxy]pyridine (INT-B11-3) as a yellow oil (59%). LCMS: m/z (ESI), [M+H]⁺=415.95. ¹H NMR (CDCl₃, 400 MHz) δ 3.42 (3H, s), 3.52-3.70 (2H, m), 3.85-4.02 (2H, m), 5.27 (2H, s), 5.33 (2H, s), 7.35-7.46 (6H, m), 7.94 (1H, s)

Step 4. Preparation of methyl 2′-(benzyloxy)-6-chloro-5′-[(2-methoxyethoxy)methoxy]-[2,4′-bipyridine]-4-carboxylate (INT-B11-4). A mixture of methyl 2-chloro-6-(4,4,5,5-tetramethyl-1,3,2-dioxaborolan-2-yl)pyridine-4-carboxylate (M1-1, 4 g crude), 2-(benzyloxy)-4-iodo-5-[(2-methoxyethoxy)methoxy]pyridine (4.1 g, 10.08 mmol, 1 equiv), Pd₂(dba)₃ (0.92 g, 1.00 mmol, 0.1 equiv), bis(adamantan-1-yl)(butyl)phosphane (0.36 g, 1.00 mmol, 0.1 equiv), and Cs₂CO₃ (9.8 g, 30.24 mmol, 3.00 equiv) in toluene/H₂O (40 mL/10 mL) was stirred for 16 h at 80 °C under nitrogen atmosphere. The mixture was cooled to room temperature. Tthe reaction was quenched with water (100 mL) and the mixture was extracted with ethyl acetate (3×100 mL). The combined organic layers were washed with brine (3×100 mL), dried over anhydrous Na₂SO₄, and concentrated under reduced pressure. The residue was purified on a silica gel column eluted with petroleum ether/ethyl acetate (1/1) to give 4.8 g of methyl 2′-(benzyloxy)-6-chloro-5′-[(2-methoxyethoxy)methoxy]-[2,4′-bipyridine]-4-carboxylate (INT-B11-4) as a brown solid (93%). LCMS: m/z (ESI), [M+H]⁺=459.05.

Step 5. Preparation of methyl 2′-(benzyloxy)-5′-[(2-methoxyethoxy)methoxy]-6-methyl-[2,4′-bipyridine]-4-carboxylate (INT-B11-5). A mixture of methyl 2′-(benzyloxy)-6-chloro-5′-[(2-methoxyethoxy)methoxy]-[2,4′-bipyridine]-4-carboxylate (2.8 g, 6.10 mmol, 1 equiv), Pd(dppf)Cl₂ (0.89 g, 1.22 mmol, 0.2 equiv), K₂CO₃ (1.6 g, 12.20 mmol, 2 equiv), PCy₃·HBF₄ (0.67 g, 1.83 mmol, 0.3 equiv), and trimethyl-1,3,5,2,4,6-trioxatriborinane (2.07 g, 16.47 mmol, 2.7 equiv) in 1,4-dioxane (28 mL) was stirred for 3 h at 100 °C under nitrogen atmosphere. The mixture was cooled to room temperature. The reaction was quenched with water (200 mL) and the mixture was extracted with ethyl acetate (3×200 mL). The combined organic layers were washed with brine (3×200 mL), dried over anhydrous Na₂SO₄, and concentrated under reduced pressure. The residue was purified on a silica gel column eluted with petroleum ether/ethyl acetate (1/1) to give 1.8 g of methyl 2′-(benzyloxy)-5′-[(2-methoxyethoxy)methoxy]-6-methyl-[2,4′-bipyridine]-4-carboxylate (INT-B11-5) as a yellow solid (60%). LCMS: m/z (ESI), [M+H]⁺=439.15.

Step 6. Preparation of methyl 2′-hydroxy-5′-[(2-methoxyethoxy)methoxy]-6-methyl-[2,4′-bipyridine]-4-carboxylate (INT-B11-6). A mixture of methyl 2′-(benzyloxy)-5′-[(2-methoxyethoxy)methoxy]-6-methyl-[2,4′-bipyridine]-4-carboxylate (1.8 g, 4.105 mmol, 1 equiv) and Pd/C (10%, 500 mg) in methanol (10 mL) was stirred for 1 h under hydrogen atmosphere. The resulting mixture was filtered and the filter cake was washed with methanol (150 mL). The solution was concentrated under reduced pressure. The residue was purified on a silica gel column eluted with dichloromethane/methanol (10/1) to give 1.3 g of methyl 2′-hydroxy-5′-[(2-methoxyethoxy)methoxy]-6-methyl-[2,4′-bipyridine]-4-carboxylate (INT-B11-6) as a yellow solid (82%). LCMS: m/z (ESI), [M+H]⁺=349.05.

Step 7. Preparation of methyl 5′-[(2-methoxyethoxy)methoxy]-1′,6-dimethyl-2′-oxo-[2,4′-bipyridine]-4-carboxylate (INT-B11-7) A mixture of methyl 2′-hydroxy-5′-[(2-methoxyethoxy)methoxy]-6-methyl-[2,4′-bipyridine]-4-carboxylate (1.1 g, 3.15 mmol, 1 equiv), K₂CO₃ (1.3 g, 9.47 mmol, 3 equiv), and CH₃I (672 mg, 4.73 mmol, 1.5 equiv) in acetone (10 mL) was stirred for 12 h at 60 °C under nitrogen atmosphere. The mixture was cooled to room temperature. The reaction was quenched with water (50 mL) and the mixture was extracted with ethyl acetate (3×50 mL). The combined organic layers were washed with brine (3×50 mL), dried over anhydrous Na₂SO₄, and concentrated under reduced pressure. The residue was purified by C18 flash using water containing 10 mmol/L NH₄HCO₃ and 0.1% NH₃—H₂O and acetonitrile as mobile phase. Fractions containing the desired compound were evaporated to dryness to give 1 g of methyl 5′-[(2-methoxyethoxy)methoxy]-1′,6-dimethyl-2′-oxo-[2,4′-bipyridine]-4-carboxylate (INT-B11-7) as a light yellow oil (83%). LCMS: m/z (ESI), [M+H]⁺=363.10. ¹H NMR (CDCl₃, 400 MHz) δ 2.66 (3H, s), 3.41 (3H, s), 3.58 (5H, d), 3.71-3.96 (2H, m), 3.96 (3H, s), 5.02 (2H, s), 6.92 (1H, s), 7.45 (1H, s), 7.73 (1H, d), 7.99 (1H, d)

Step 8. Preparation of methyl 5′-hydroxy-1′,6-dimethyl-2′-oxo-[2,4′-bipyridine]-4-carboxylate (INT-B11-8). A solution of methyl 5′-[(2-methoxyethoxy)methoxy]-1′,6-dimethyl-2′-oxo-[2,4′-bipyridine]-4-carboxylate (370 mg, 1.02 mmol, 1 equiv) and HCl (10 mL, 4M in MeOH) was stirred for 1.5 h at room temperature under nitrogen atmosphere. The mixture was concentrated under reduced pressure. The residue was treated with dichloromethane (30 mL) and NaHCO₃ solution and filtered. The filtrate was concentrated under reduced pressure to give 260 mg of methyl 5′-hydroxy-1′,6-dimethyl-2′-oxo-[2,4′-bipyridine]-4-carboxylate (INT-B11-8) as a yellow solid. This material was used for the next reaction without further purification. LCMS: m/z (ESI), [M+H]⁺=275.05.

Step 9. Preparation of methyl 1′,6-dimethyl-5′-{[(4R)-4-methyl-5-[(2-nitrophenyl)amino]pentyl]oxy}-2′-oxo-[2,4′-bipyridine]-4-carboxylate (INT-B11-9). A mixture of 5′-hydroxy-1′,6-dimethyl-2′-oxo-[2,4′-bipyridine]-4-carboxylate (282 mg, 1.03 mmol, 1 equiv), (4R)-4-methyl-5-[(2-nitrophenyl)amino]pentan-1-ol (247 mg, 1.03 mmol, 1 equiv), and 2-(tributyl-1\hat{ }[5]-phosphanylidene)acetonitrile (1.00 g, 4.14 mmol, 4.00 equiv) in toluene (10 mL) was stirred for 2 h at 100 °C under nitrogen atmosphere. The mixture was cooled to room temperature. The reaction was quenched with water (100 mL) and the mixture was extracted with dichloromethane (3×100 mL). The combined organic layers were washed with brine (3×100 mL), dried over anhydrous Na₂SO₄, and concentrated under reduced pressure. The residue was purified on a silica gel column eluted with petroleum ether/ethyl acetate (1/1) to give 400 mg of methyl 1′,6-dimethyl-5′-{[(4R)-4-methyl-5-[(2-nitrophenyl)amino]pentyl]oxy}-2′-oxo-[2,4′-bipyridine]-4-carboxylate (INT-B11-9) as a yellow oil (77%). LCMS: m/z (ESI), [M+H]⁺=495.10.

Step 10. Preparation of methyl 5′-{[(4R)-5-[(2-aminophenyl)amino]-4-methylpentyl]oxy}-1′,6-dimethyl-2′-oxo-[2,4′-bipyridine]-4-carboxylate (INT-B11-10). To a stirred mixture of methyl 1′,6-dimethyl-5′-{[(4R)-4-methyl-5-[(2-nitrophenyl)amino]pentyl]oxy}-2′-oxo-[2,4′-bipyridine]-4-carboxylate (400 mg, 0.80 mmol, 1 equiv) and Raney Nickel (34 mg, 0.40 mmol, 0.50 equiv) in methanol (10 mL) was added hydrazine hydrate (77 mg, 2.42 mmol, 3.00 equiv) at 0 °C. The mixture was stirred for 2 h at 0 °C under nitrogen atmosphere. The mixture was filtered and the filter cake was washed with methanol (3×10 mL). The resulting solution was concentrated under reduced pressure. The residue was purified on a prep-TLC eluted with petroleum ether/ethyl acetate (1/1) to give 350 mg of methyl 5′-{[(4R)-5-[(2-aminophenyl)amino]-4-methylpentyl]oxy}-1′,6-dimethyl-2′-oxo-[2,4′-bipyridine]-4-carboxylate (INT-B11-10) as a yellow solid (93%). LCMS: m/z (EST), [M+H]⁺=465.20.

Step 11. Preparation of methyl 5′-{[(4R)-4-[(2-amino-1,3-benzodiazol-1-yl)methyl]pentyl]oxy}-1′,6-dimethyl-2′-oxo-[2,4′-bipyridine]-4-carboxylate (INT-B11-11). A mixture of methyl 5′-{[(4R)-5-[(2-aminophenyl)amino]-4-methylpentyl]oxy}-1′,6-dimethyl-2′-oxo-[2,4′-bipyridine]-4-carboxylate (400 mg, 0.86 mmol, 1 equiv) and BrCN (182 mg, 1.722 mmol, 2.00 equiv) in ethanol (10 mL) was stirred overnight at room temperature under nitrogen atmosphere. The reaction was quenched with water (50 mL) and the mixture was extracted with ethyl acetate (3×50 mL). The combined organic layers were washed with brine (3×50 mL), dried over anhydrous Na₂SO₄, and concentrated under reduced pressure. The residue was purified on a prep-TLC eluted with dichloromethane/methanol (20/1) to give 350 mg of methyl 5′-{[(4R)-4-[(2-amino-1,3-benzodiazol-1-yl)methyl]pentyl]oxy}-1′,6-dimethyl-2′-oxo-[2,4′-bipyridine]-4-carboxylate (INT-B11-11) as a yellow oil (83%). LCMS: m/z (ESI), [M+H]⁺=490.10.

Step 12. Preparation of 5′-{[(4R)-4-[(2-amino-1,3-benzodiazol-1-yl)methyl]pentyl]oxy}-1′,6-dimethyl-2′-oxo-[2,4′-bipyridine]-4-carboxylic acid (INT-B11-12). A mixture of methyl 5′-{[(4R)-4-[(2-amino-1,3-benzodiazol-1-yl)methyl]pentyl]oxy}-1′,6-dimethyl-2′-oxo-[2,4′-bipyridine]-4-carboxylate (300 mg, 0.61 mmol, 1 equiv) and LiOH—H₂O (44 mg, 1.83 mmol, 3 equiv) in tetrahydrofuran/water (4 mL/1 mL) was stirred for 2 h at room temperature under nitrogen atmosphere. The resulting mixture was concentrated under reduced pressure. The residue was purified by C18 flash chromatography using water containing 0.10% NH₃·H₂O and acetonitrile as mobile phase. Fractions containing the desired compound were evaporated to dryness to give 200 mg of 5′-{[(4R)-4-[(2-amino-1,3-benzodiazol-1-yl)methyl]pentyl]oxy}-1′,6-dimethyl-2′-oxo-[2,4′-bipyridine]-4-carboxylic acid (INT-B11-12) as a yellow solid (68%). LCMS: m/z (ESI), [M+H]⁺=476.10.

Step 13. Preparation of (12R)-5,12,27-trimethyl-8-oxa-5,14,21,23,28-pentaazapentacyclo[23.3.1.0\hat{ }{2,7}.0\hat{ }{14,22}.0\hat{ }{15,20}]nonacosa-1(29),2,6,15,17,19,21,25,27-nonaene-4,24-dione (EXAMPLE B11). A mixture of 5′-{[(4R)-4-[(2-amino-1,3-benzodiazol-1-yl)methyl]pentyl]oxy}-1′,6-dimethyl-2′-oxo-[2,4′-bipyridine]-4-carboxylic acid (400 mg, 0.84 mmol, 1 equiv), N,N,N,N-tetramethyl-O-(7-azabenzotriazol-1-yl)uronium hexafluorophospate (479 mg, 1.26 mmol, 1.5 equiv) and N,N-diisopropylethylamine (217 mg, 1.68 mmol, 2 equiv) in 1,4-dioxane (10 mL) was stirred overnight at room temperature under nitrogen atmosphere. The reaction was quenched with water (100 mL) and the mixture was extracted with dichloromethane (3×100 mL). The combined organic layers were washed with brine (3×100 mL), dried over anhydrous Na₂SO₄, and concentrated under reduced pressure. The residue was purified by prep-HPLC with XBridge Shield RP18 OBD column using water containing 10 mmol/L NH₄HCO₃ and 0.1% NH₃—H₂O and acetonitrile as mobile phase. Fractions containing the desired compound were evaporated to dryness to give 186 mg of (12R)-5,12,27-trimethyl-8-oxa-5,14,21,23,28-pentaazapentacyclo[23.3.1.0\hat{ }{2,7}.0\hat{ }{14,22}.0\hat{ }{15,20}]nonacosa-1(29),2,6,15,17,19,21,25,27-nonaene-4,24-dione (EXAMPLE B11) as a white solid (48%). LCMS: m/z (ESI), [M+H]⁺=458.25. ¹H NMR (DMSO-d₆, 400 MHz) δ 0.93 (3H, d), 1.47-1.60 (1H, m), 1.66-1.80 (1H, m), 1.96-2.20 (2H, m), 2.25-2.37 (1H, m), 2.63 (3H, s), 3.48 (3H, s), 3.93-4.08 (3H, m), 4.18 (1H, dd), 7.12 (1H, s), 7.19-7.34 (2H, m), 7.53-7.61 (3H, m), 7.83 (1H, d), 8.92 (1H, d), 12.80 (1H, s).

### Example B12

### (11R)-18-[2-(dimethylamino)ethyl]-5,11,30-trimethyl-7-oxa-4,5,13,18,24,26,31-heptaazahexacyclo[26.3.1.0\hat{ }{2,6}.0\hat{ }{13,25}.0\hat{ }{14,23}.0\hat{ }{15,20}]dotriaconta-1(31),2(6),3,14,20,22,24,28(32),29-nonaen-27-one

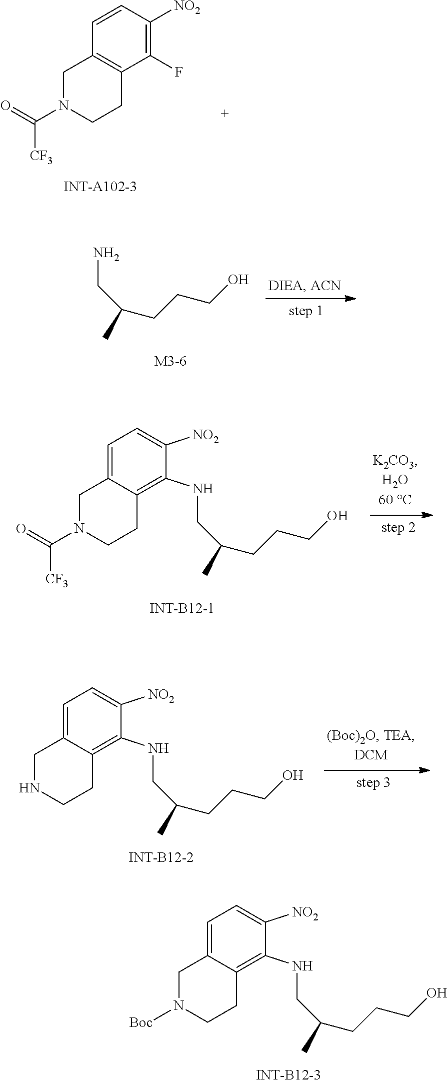

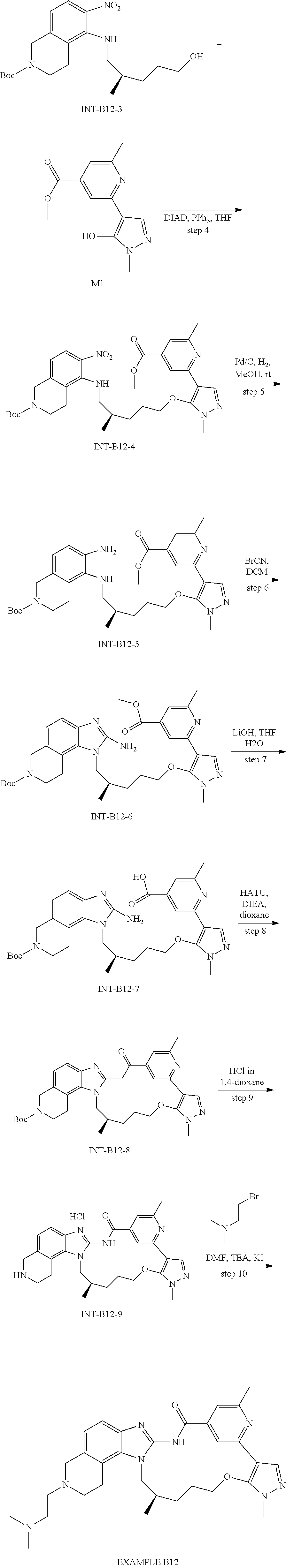

Step 1. Preparation of 2,2,2-trifluoro-1-(5-{[(2R)-5-hydroxy-2-methylpentyl]amino}-6-nitro-3,4-dihydro-1H-isoquinolin-2-yl)ethanone (INT-B12-1). A mixture of 2,2,2-trifluoro-1-(5-fluoro-6-nitro-3,4-dihydro-1H-isoquinolin-2-yl)ethanone (INT-A102-3, 15 g, 51.34 mmol, 1 equiv), N, N-diisopropylethylamine (19.9 g, 154.01 mmol, 3 equiv) and (4R)-5-amino-4-methylpentan-1-ol (M3-6,6016 mg, 51.34 mmol, 1 equiv) in acetonitrile (150 mL) was stirred for 2 h at 80 °C. The mixture was cooled to room temperature. The reaction was quenched with water (500 mL) and the mixture was extracted with dichloromethane (3×500 mL). The combined organic layers were washed with brine (3×100 mL), dried over anhydrous Na₂SO₄, and concentrated under reduced pressure. The residue was purified by a silica gel column eluted with petroleum ether/ethyl acetate (1/1) to give 10 g of 2,2,2-trifluoro-1-(5-{[(2R)-5-hydroxy-2-methylpentyl]amino}-6-nitro-3,4-dihydro-L H-isoquinolin-2-yl)ethanone (INT-B12-1) as a yellow solid (50%). LCMS: m/z (ESI), [M+H]⁺=390.10.

Step 2. Preparation of (4R)-4-methyl-5-[(6-nitro-1,2,3,4-tetrahydroisoquinolin-5-yl)amino]pentan-1-ol (INT-B12-2). A mixture of 2,2,2-trifluoro-1-(5-{[(2R)-5-hydroxy-2-methylpentyl]amino}-6-nitro-3,4-dihydro-1H-isoquinolin-2-yl)ethanone (10 g, 25.68 mmol, 1 equiv), and K₂CO₃ (10.6 g, 77.05 mmol, 3 equiv) in methanol (80 mL) and H₂O (40 mL) was stirred for 5 h at room temperature. The reaction was treated with water (500 mL) and the mixture was extracted with dichloromethane (3×500 mL). The combined organic layers were washed with brine (3×100 mL), dried over anhydrous Na₂SO₄, and concentrated under reduced pressure. The residue was purified on a silica gel column eluted with petroleum ether/ethyl acetate (1/1) to give 7 g of (4R)-4-methyl-5-[(6-nitro-1,2,3,4-tetrahydroisoquinolin-5-yl)amino]pentan-1-ol (INT-B12-2) as a yellow solid (93%). LCMS: m/z (ESI), [M+H]⁺=294.15. ¹H NMR (DMSO-d₆,400 MHz) δ 0.83-0.88 (3H, m), 1.07-1.11 (1H, m), 1.22-1.51 (3H, m), 1.57-1.64 (1H, m), 2.59 (2H, t), 2.88-2.92 (3H, m), 3.10-3.20 (1H, m), 3.30-3.33 (2H, m), 3.87 (2H, s), 4.37 (1H, s), 6.56 (1H, d), 6.69 (1H, t), 7.26-7.37 (1H, m), 7.70 (1H, d).

Step 3. Preparation of tert-butyl 5-{[(2R)-5-hydroxy-2-methylpentyl]amino}-6-nitro-3,4-dihydro-1H-isoquinoline-2-carboxylate (INT-B12-3). A mixture of (4R)-4-methyl-5-[(6-nitro-1,2,3,4-tetrahydroisoquinolin-5-yl)amino]pentan-1-ol (8 g, 27.27 mmol, 1 equiv), trimethylamine (5.5 g, 54.54 mmol, 2 equiv) and di-tert-butyl dicarbonate (7.1 g, 32.72 mmol, 1.2 equiv) in dichloromethane (100 mL) was stirred for 2 h at room temperature. The reaction was quenched with water (300 mL) and the mixture was extracted with dichloromethane (3×300 mL). The combined organic layers were washed with brine (3×100 mL), dried over anhydrous Na₂SO₄, and concentrated under reduced pressure. The residue was purified on silica gel column eluted with petroleum ether/ethyl acetate (1/1) to give 6.5 g of tert-butyl 5-{[(2R)-5-hydroxy-2-methylpentyl]amino}-6-nitro-3,4-dihydro-1H-isoquinoline-2-carboxylate (INT-B12-3) as a yellow oil (60%). LCMS: m/z (ESI), [M+H]+=394.20. ¹H NMR (DMSO-d₆ 400 MHz) δ 0.84 (3H, d), 1.03-1.17 (1H, m), 1.22-1.45 (2H, m), 1.45 (9H, s), 1.61 (1H, q), 2.75 (2H, t), 2.93-2.97 (1H, m), 3.08-3.12 (1H, m), 3.30-3.33 (2H, m), 3.51 (2H, t), 4.36 (1H, t), 4.52 (2H, s), 6.73 (1H, d), 6.83 (1H, s), 7.80 (1H, d)

Step 4. Preparation of tert-butyl 5-{[(2R)-5-({4-[4-(methoxycarbonyl)-6-methylpyridin-2-yl]-2-methylpyrazol-3-yl}oxy)-2-methylpentyl]amino}-6-nitro-3,4-dihydro-1H-isoquinoline-2-carboxylate (INT-B12-4). To a stirred mixture of tert-butyl 5-{[(2R)-5-hydroxy-2-methylpentyl]amino}-6-nitro-3,4-dihydro-1H-isoquinoline-2-carboxylate (1 g, 2.54 mmol, 1 equiv) and triphenylphosphine (2 g, 7.62 mmol, 3 equiv) in tetrahydrofuran (20 mL) were added methyl 2-(5-hydroxy-1-methylpyrazol-4-yl)-6-methylpyridine-4-carboxylate (M1, 691 mg, 2.79 mmol, 1.1 equiv) and diisopropyl azodicarboxylate (1.5 g, 7.62 mmol, 3 equiv) at 0 °C under nitrogen atmosphere. The mixture was stirred for 2 h at room temperature. The reaction was quenched with water (100 mL) and the mixture was extracted with dichloromethane (3×80 mL). The combined organic layers were washed with brine (3×80 mL), dried over anhydrous Na₂SO₄, and concentrated under reduced pressure. The residue was purified on a silica gel column eluted with petroleum ether/ethyl acetate (2/3) to give 900 mg of tert-butyl 5-{[(2R)-5-({4-[4-(methoxycarbonyl)-6-methylpyridin-2-yl]-2-methylpyrazol-3-yl}oxy)-2-methylpentyl]amino}-6-nitro-3,4-dihydro-1H-isoquinoline-2-carboxylate (INT-B12-4) as a light yellow solid (57%). LCMS: m/z (ESI), [M+H]⁺=623.30.

Step 5. Preparation of tert-butyl 6-amino-5-{[(2R)-5-({4-[4-(methoxycarbonyl)-6-methylpyridin-2-yl]-2-methylpyrazol-3-yl}oxy)-2-methylpentyl]amino}-3,4-dihydro-1H-isoquinoline-2-carboxylate (INT-B12-5). A mixture of tert-butyl 5-{[(2R)-5-({4-[4-(methoxycarbonyl)-6-methylpyridin-2-yl]-2-methylpyrazol-3-yl}oxy)-2-methylpentyl]amino}-6-nitro-3,4-dihydro-1H-isoquinoline-2-carboxylate (500 mg, 0.80 mmol, 1 equiv) and Pd/C (10%, 30 mg) in methanol (10 mL) was stirred for 1 h under hydrogen atmosphere. The resulting mixture was filtered and the filter cake was washed with methanol (3×50 mL). The resulting solution was concentrated under reduced pressure.

The residue was purified by prep-TLC eluted with dichloromethane/methanol (10/1) to give 360 mg of tert-butyl 6-amino-5-{[(2R)-5-({4-[4-(methoxycarbonyl)-6-methylpyridin-2-yl]-2-methylpyrazol-3-yl}oxy)-2-methylpentyl]amino}-3,4-dihydro-1H-isoquinoline-2-carboxylate (INT-B12-5) as a light yellow solid (76%). LCMS: m/z (ESI), [M+H]*=593.40.

Step 6. Preparation of methyl 2-(5-{[(4R)-4-{[2-amino-7-(tert-butoxycarbonyl)-6H,8H,9H-imidazo[4,5-f]isoquinolin-1-yl]methyl}pentyl]oxy}-1-methylpyrazol-4-yl)-6-methylpyridine-4-carboxylate (INT-B12-6). A mixture of tert-butyl 6-amino-5-{[(2R)-5-({4-[4-(methoxycarbonyl)-6-methylpyridin-2-yl]-2-methylpyrazol-3-yl}oxy)-2-methylpentyl]amino}-3,4-dihydro-1H-isoquinoline-2-carboxylate (350 mg, 0.59 mmol, 1 equiv) and BrCN (75 mg, 0.71 mmol, 1.2 equiv) in dichloromethane (4 mL) was stirred for 2 h at room temperature under nitrogen atmosphere. The reaction was quenched with water (60 mL) and the mixture was extracted with dichloromethane (3×60 mL). The combined organic layers were washed with brine (3×50 mL), dried over anhydrous Na₂SO₄, and concentrated under reduced pressure. The residue was purified by prep-TLC eluted with dichloromethane/methanol (11/1) to give 300 mg of methyl 2-(5-{[(4R)-4-{[2-amino-7-(tert-butoxycarbonyl)-6H,8H,9H-imidazo[4,5-f]isoquinolin-1-yl]methyl}pentyl]oxy}-1-methylpyrazol-4-yl)-6-methylpyridine-4-carboxylate (INT-B12-6) as a yellow oil (82%). LCMS: m/z (EST), [M+H]⁺=618.35.

Step 7. Preparation of 2-(5-{[(4R)-4-{[2-amino-7-(tert-butoxycarbonyl)-6H,8H,9H-imidazo[4,5-f]isoquinolin-1-yl]methyl}pentyl]oxy}-1-methylpyrazol-4-yl)-6-methylpyridine-4-carboxylic acid (INT-B12-7). A mixture of methyl 2-(5-{[(4R)-4-{[2-amino-7-(tert-butoxycarbonyl)-6H,8H,9H-imidazo[4,5-f]isoquinolin-1-yl]methyl}pentyl]oxy}-1-methylpyrazol-4-yl)-6-methylpyridine-4-carboxylate (290 mg, 0.47 mmol, 1 equiv) and LiOH (30 mg, 0.70 mmol, 1.5 equiv) in tetrahydrofuran/H₂O (4 mL/1 mL) was stirred for 2 h at room temperature. The mixture was concentrated in vacuo. The residue was purified by C18 flash chromatography using water and acetonitrile as mobile phase. Fractions containing the desired compound were evaporated to dryness to give 900 mg of 2-(5-{[(4R)-4-{[2-amino-7-(tert-butoxycarbonyl)-6H,8H,9H-imidazo[4,5-f]isoquinolin-1-yl]methyl}pentyl]oxy}-1-methylpyrazol-4-yl)-6-methylpyridine-4-carboxylic acid (INT-B12-7) as a red solid (71%). LCMS: m/z (ESI), [M+H]⁺=604.30.

Step 8. Preparation of tert-butyl (11R)-5,11,30-trimethyl-27-oxo-7-oxa-4,5,13,18,24,26,31-heptaazahexacyclo[26.3.1.0\hat{ }{2,6}.0\hat{ }{13,25}.0\hat{ }{14,23}.0\hat{ }{15,20}]dotriaconta-1(31),2(6),3,14,20,22,24,28(32),29-nonaene-18-carboxylate (INT-B12-8). A mixture of 2-(5-{[(4R)-4-{[2-amino-7-(tert-butoxycarbonyl)-6H,8H,9H-imidazo[4,5-f]isoquinolin-1-yl]methyl}pentyl]oxy}-1-methylpyrazol-4-yl)-6-methylpyridine-4-carboxylic acid (200 mg, 0.33 mmol, 1 equiv), N,N-diisopropylethylamine (128 mg, 0.99 mmol, 3 equiv) and N,N,N,N-tetramethyl-O-(7-azabenzotriazol-1-yl)uronium hexafluorophospate (252 mg, 0.66 mmol, 2 equiv) in dioxane (5 mL) was stirred for 2 h at room temperature under nitrogen atmosphere. The reaction was quenched with water (60 mL) and the mixture was extracted with dichloromethane (3×60 mL). The combined organic layers were washed with brine (3×40 mL), dried over anhydrous Na₂SO₄, and concentrated under reduced pressure. The residue was purified by prep-TLC eluted with dichloromethane/methanol (15/1) to give 160 mg of tert-butyl (11R)-5,11,30-trimethyl-27-oxo-7-oxa-4,5,13,18,24,26,31-heptaazahexacyclo[26.3.1.0\hat{ }{2,6}.0\hat{ }{13,25}.0\hat{ }{14,23}.0\hat{ }{15,20}]dotriaconta-1(31),2(6),3,14,20,22,24,28(32),29-nonaene-18-carboxylate (INT-B12-8) as a yellow solid (82%). LCMS: m/z (ESI), [M+H]⁺=586.30.

Step 9. Preparation of (11R)-5,11,30-trimethyl-7-oxa-4,5,13,18,24,26,31-heptaazahexacyclo[26.3.1.0\hat{ }{2,6}.0\hat{ }{13,25}.0\hat{ }{14,23}.0\hat{ }{15,20}]dotriaconta-1(31),2(6),3,14,20,22,24,28(32),29-nonaen-27-one (INT-B12-9). To a stirred mixture of tert-butyl (11R)-5,11,30-trimethyl-27-oxo-7-oxa-4,5,13,18,24,26,31-heptaazahexacyclo[26.3.1.0\hat{ }{2,6}.0\hat{ }{13,25}.0\hat{ }{14,23}.0\hat{ }{15,20}]dotriaconta-1(31),2(6),3,14,20,22,24,28(32),29-nonaene-18-carboxylate (120 mg, 0.21 mmol, 1 equiv) in 1,4-dioxane (0.5 mL) was added HCl solution (3 mL, 4M in 1,4-dioxane) at room temperature. The mixture was stirred for 2 h at room temperature. The resulting mixture was treated with saturated NaHCO₃ (aq.) (100 mL) and extracted with dichloromethane (3×100 mL). The combined organic layers were washed with brine (3×70 mL), dried over anhydrous Na₂SO₄, and concentrated under reduced pressure to give 99 mg of (11R)-5,11,30-trimethyl-7-oxa-4,5,13,18,24,26,31-heptaazahexacyclo[26.3.1.0\hat{ }{2,6}.0\hat{ }{13,25}.0\hat{ }{14,23}.0\hat{ }{15,20}]dotriaconta-1(31),2(6),3,14,20,22,24,28(32),29-nonaen-27-one (INT-B12-9) as a yellow solid (99%). This material was used for the next step without purification. LCMS: m/z (ESI), [M+H]=486.30.

Step 10. Preparation of (11R)-18-[2-(dimethylamino)ethyl]-5,11,30-trimethyl-7-oxa-4,5,13,18,24,26,31-heptaazahexacyclo[26.3.1.0\hat{ }{2,6}.0\hat{ }{13,25}.0\hat{ }{14,23}.0\hat{ }{15,20}1]dotriaconta-1(31),2(6),3,14,20,22,24,28(32),29-nonaen-27-one (EXAMPLE B12). To a stirred mixture of (11R)-5,11,30-trimethyl-7-oxa-4,5,13,18,24,26,31-heptaazahexacyclo[26.3.1.0\hat{ }{2,6}.0\hat{ }{13,25}.0\hat{ }{14,23}.0\hat{ }{15,20}]dotriaconta-1(31),2(6),3,14,20,22,24,28(32),29-nonaen-27-one (99 mg, 0.20 mmol, 1 equiv) and trimethylamine (62 mg, 0.61 mmol, 3 equiv) in N,N-dimethylformamide (5 mL) were added (2-bromoethyl)dimethylamine (56 mg, 0.37 mmol, 1.8 equiv) and KI (10 mg, 0.06 mmol, 0.3 equiv) at room temperature. The resulting mixture was stirred for 2 h at 80 °C under nitrogen atmosphere. The mixture was cooled to room temperature. The reaction was quenched with water (50 mL) and the mixture was extracted with dichloromethane (3×60 mL). The combined organic layers were washed with brine (3×40 mL), dried over anhydrous Na₂SO₄, and concentrated under reduced pressure. The residue was purified by prep-TLC eluted with dichloromethane/methanol (20/1). The product was further purified by prep-HPLC with XBridge Prep OBD C18 column using water containing 10 mmol/L NH₄HCO₃ and acetonitrile as mobile phase. Fractions containing the desired compound were evaporated to dryness to give 9.3 mg of (11R)-18-[2-(dimethylamino)ethyl]-5,11,30-trimethyl-7-oxa-4,5,13,18,24,26,31-heptaazahexacyclo[26.3.1.0\hat{ }{2,6}.0\hat{ }{13,25}.0\hat{ }{14,23}.0\hat{ }{15,20}]dotriaconta-1(31),2(6),3,14,20,22,24,28(32),29-nonaen-27-one (EXAMPLE B12) as a yellow solid (8%). LCMS: m/z (ESI), [M+H]⁺=557.30. ¹H NMR (DMSO-d₆, 400 MHz) δ 0.85 (3H, d), 1.41-1.53 (1H, m), 1.77-1.92 (1H, m), 1.95-2.08 (1H, m), 2.19 (6H, s), 2.21-2.29 (1H, m), 2.49-2.61 (5H, m), 2.72-2.86 (2H, m), 2.88-3.02 (1H, m), 3.10-3.27 (2H, m), 3.60-3.70 (2H, m), 3.73 (3H, s), 3.96-4.02 (1H, m), 4.22-4.28 (4H, m), 6.95 (1H, d), 7.39 (1H, d), 7.57 (1H, s), 7.93 (1H, s), 8.36 (1H, d), 12.75 (1H, s).

### Example B13

### (R)-11,26,7-trimethyl-57-(oxetan-3-yl)-56,57,58,59-tetrahydro-11H,51H-11-oxa-4-aza-5(2,1)-imidazo[4,5-f]isoquinolina-2(2,4)-pyridina-1(4,5)-pyrazolacycloundecaphan-3-one

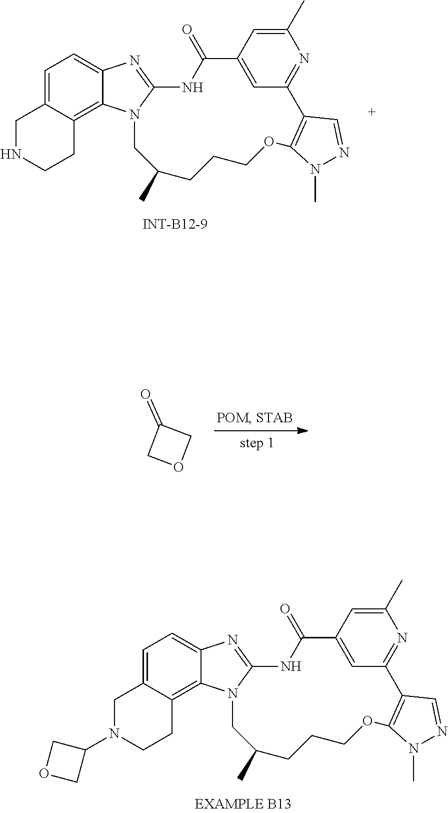 [SMILES: Cc1cc2cc(n1)-c1cnn(C)c1OCCC[C@@H](C)Cn1c(nc3ccc4c(c31)CCNC4)NC2=O ; O=C1COC1 ; Cc1cc2cc(n1)-c1cnn(C)c1OCCC[C@@H](C)Cn1c(nc3ccc4c(c31)CCN(C1COC1)C4)NC2=O]

Step 1. Preparation of (11R)-11,26,7-trimethyl-57-(oxetan-3-yl)-56,57,58,59-tetrahydro-11H,51H-11-oxa-4-aza-5(2,1)-imidazo[4,5-f]isoquinolina-2(2,4)-pyridina-1(4,5)-pyrazolacycloundecaphan-3-one (EXAMPLE B13). A mixture of (11R)-5,11,30-trimethyl-7-oxa-4,5,13,18,24,26,31-heptaazahexacyclo[26.3.1.0\hat{ }{2,6}.0\hat{ }{13,25}.0\hat{ }{14,23}.0\hat{ }{15,20}1]dotriaconta-1(31),2(6),3,14,20,22,24,28(32),29-nonaen-27-one (INT-B12-9, 40 mg, 0.08 mmol, 1 equiv) in dichloromethane (2 mL) was treated with 3-oxetanone (12 mg, 0.16 mmol, 2 equiv) and trimethylamine (17 mg, 0.16 mmol, 2 equiv) for 0.5 h at room temperature under nitrogen atmosphere followed by the addition of NaBH(AcO)₃ (52 mg, 0.25 mmol, 3 equiv) at 0 °C. The resulting mixture was stirred overnight at room temperature under nitrogen atmosphere. The reaction was quenched with water (20 mL) and the mixture was extracted with dichloromethane (3×20 mL). The combined organic layers were washed with brine (3×20 mL), dried over anhydrous Na₂SO₄, and concentrated under reduced pressure. The residue was purified by prep-HPLC with XBridge Shield RP18 OBD column using water containing 10 mmol/L NH₄HCO₃ and 0.1% NH₃·H₂O and acetonitrile as mobile phase. Fractions containing the desired compound were evaporated to dryness to give 10.5 mg of (11R)-5,11,30-trimethyl-18-(oxetan-3-yl)-7-oxa-4,5,13,18,24,26,31-heptaazahexacyclo[26.3.1.0\hat{ }{2,6}.0\hat{ }{13,25}.0\hat{ }{14,23}.0\hat{ }{15,20}]dotriaconta-1(31),2(6),3,14,20,22,24,28(32),29-nonaen-27-one (EXAMPLE B13) as a white solid (22%). LCMS: m/z (ESI), [M+H]⁺=542.20. ¹H NMR (DMSO-d₆, 400 MHz) δ 0.86 (3H, d), 1.38-1.53 (1H, m), 1.79-1.93 (1H, m), 1.96-2.06 (1H, m), 2.16-2.28 (1H, m), 2.58 (3H, s), 2.58-2.73 (3H, m), 2.90-3.02 (1H, m), 3.15-3.26 (1H, m), 3.45-3.64 (3H, m), 3.73 (3H, s), 3.97-4.01 (1H, m), 4.14-4.38 (3H, m), 4.56 (2H, t), 4.65 (2H, t), 6.96 (1H, d), 7.41 (1H, d), 7.58 (1H, s), 7.93 (1H, s), 8.39 (1H, s), 12.78 (1H, s).

### Example B14

### 30-Methyl-11-oxa-8,9,17,24,26,31-hexaazahexacyclo[26.3.1.0\hat{ }{2,10}.0\hat{ }{3,8}.0\hat{ }{17,25}.0\hat{ }{18,23}]dotriaconta-1(31),2,4,6,9,18,20,22,24,28(32),29-undecaen-27-one

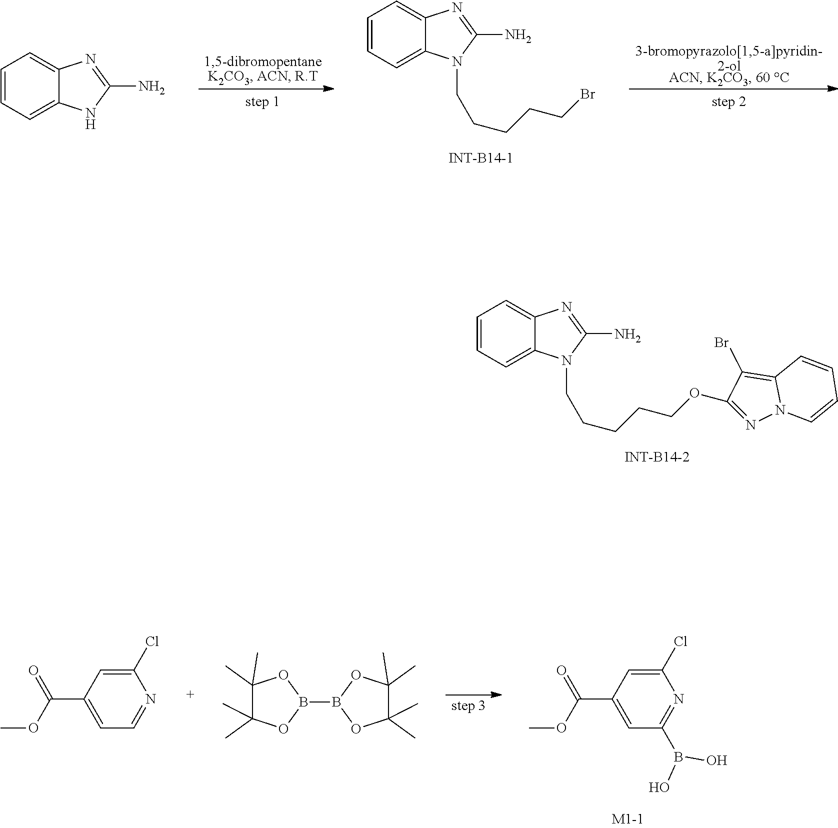

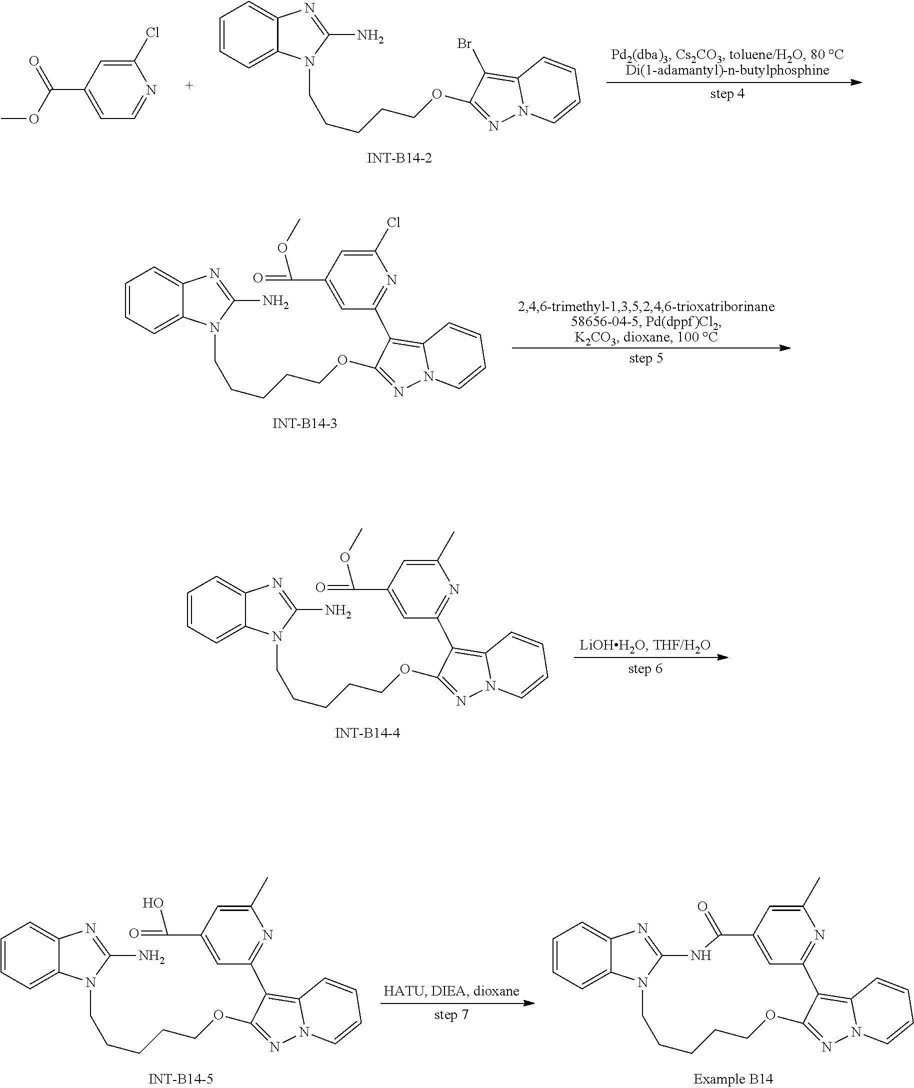

Step 1. Preparation of 1-(5-bromopentyl)-1,3-benzodiazol-2-amine (INT-B14-1). A mixture of 2-aminobenzimidazole (4 g, 30.04 mmol, 1 equiv), K₂CO₃ (12.46 g, 90.12 mmol, 3 equiv), and 1,5-dibromopentane (27.63 g, 120.16 mmol, 4 equiv) in acetonitrile (80 mL) was stirred for 5 h at 60 °C. The mixture was cooled to room temperature. The reaction was quenched with water (500 mL) and the mixture was extracted with ethyl acetate (3×500 mL). The combined organic layers were washed with brine (3×500 mL), dried over anhydrous Na₂SO₄, and concentrated under reduced pressure. The residue was purified on a silica gel column eluted with dichloromethane/methanol (10/1) to give 4 g of 1-(5-bromopentyl)-1,3-benzodiazol-2-amine (INT-B14-1) as a brown solid (47%). LCMS: m/z (ESI), [M+H]⁺=283.95.

Step 2. Preparation of 1-[5-({3-bromopyrazolo[1,5-a]pyridin-2-yl}oxy)pentyl]-1,3-benzodiazol-2-amine (INT-B14-2). A mixture of 1-(5-bromopentyl)-1,3-benzodiazol-2-amine (3.97 g, 14.08 mmol, 1.5 equiv), 3-bromopyrazolo[1,5-a]pyridin-2-ol (2 g, 9.38 mmol, 1.00 equiv), and K₂CO₃ (2.59 g, 18.77 mmol, 2 equiv) in acetonitrile (80 mL) was stirred for 2 h at 60 °C. The mixture was cooled to room temperature. The reaction was quenched with water (500 mL) and the mixture was extracted with ethyl acetate (3×500 mL). The combined organic layers were washed with brine (3×500 mL), dried over anhydrous Na₂SO₄, and concentrated under reduced pressure. The residue was purified by C18 flash chromatography using water and acetonitrile as mobile phase. Fractions containing the desired compound were evaporated to dryness to give 2.3 g of 1-[5-({3-bromopyrazolo[1,5-a]pyridin-2-yl}oxy)pentyl]-1,3-benzodiazol-2-amine (INT-B14-2) as a brown solid (39%). LCMS: m/z (EST), [M+H]⁺=414.20. ¹H NMR (DMSO-d₆, 400 MHz) δ 1.40-1.52 (2H, m), 1.63.175 (2H, m), 1.76-1.88 (2H, m), 3.92-4.03 (2H, m), 4.30 (2H, t), 6.37 (2H, s), 6.79-6.87 (2H, m), 6.88-6.94 (1H, m), 7.12 (2H, t), 7.26-7.39 (2H, m), 8.54 (1H, d).

Step 3. Preparation of 6-chloro-4-(methoxycarbonyl)pyridin-2-ylboronic acid) (M1-1). A mixture of bis(pinacolato)diboron (1631.71 mg, 6.426 mmol, 1.5 equiv) in tert-butyl methyl ether (20 mL) warmed to 80 °C and stirred for 0.5 h under nitrogen atmosphere, then cooled to room temperature. To this mixture were added bis(1,5-cyclooctadiene)di-p-methoxydiiridium(I) (283.95 mg, 0.428 mmol, 0.1 equiv) and 4-tert-butyl-2-(4-tert-butylpyridin-2-yl)pyridine (229.95 mg, 0.857 mmol, 0.2 equiv). The mixture was stirred for 0.5 h at 25 °C and then added methyl 2-chloropyridine-4-carboxylate (735 mg, 4.284 mmol, 1 equiv). The reaction mixture was continued to stir for 16 h at 80 °C. The mixture was cooled to room temperature and concentrated under reduced pressure to give 850 mg (crude) of 6-chloro-4-(methoxycarbonyl)pyridin-2-ylboronic acid (M1-1) as a brown oil. This material was used for the next reaction without further purification. LCMS: m/z (EST), [M+H]⁺=216.05.

Step 4. methyl 2-(2-{[5-(2-amino-1,3-benzodiazol-1-yl)pentyl]oxy}pyrazolo[1,5-a]pyridin-3-yl)-6-chloropyridine-4-carboxylate (INT-B14-3). A mixture of freshly prepared 6-chloro-4-(methoxycarbonyl)pyridin-2-ylboronic acid (850 mg crude, ~2.5 equiv), 1-[5-({3-bromopyrazolo[1,5-a]pyridin-2-yl}oxy)pentyl]-1,3-benzodiazol-2-amine (654 mg, 1.58 mmol, 1.00 equiv), Cs₂CO₃ (1543 mg, 4.73 mmol, 3 equiv), Pd₂(dba)₃ (144 mg, 0.15 mmol, 0.1 equiv), and bis(adamantan-1-yl)(butyl)phosphane (57 mg, 0.16 mmol, 0.1 equiv) in H₂O (4 mL) and toluene (20 mL) was stirred for 2 h at 80 °C under nitrogen atmosphere. The mixture was cooled to room temperature. The reaction was quenched with water (200 mL) and the mixture was extracted with ethyl acetate (3×200 mL). The combined organic layers were washed with brine (3×200 mL), dried over anhydrous Na₂SO₄, and concentrated under reduced pressure. The residue was purified on a silica gel column eluted with petroleum ether/ethyl acetate (1/3) to give 250 mg of methyl 2-(2-{[5-(2-amino-1,3-benzodiazol-1-yl)pentyl]oxy}pyrazolo[1,5-a]pyridin-3-yl)-6-chloropyridine-4-carboxylate (INT-B14-3) as a brown solid (31%). LCMS: m/z (ESI), [M+H]⁺=505.15

Step 5. Preparation of methyl 2-(2-{[5-(2-amino-1,3-benzodiazol-1-yl)pentyl]oxy}pyrazolo[1,5-a]pyridin-3-yl)-6-methylpyridine-4-carboxylate (INT-B14-4). A mixture of methyl 2-(2-{[5-(2-amino-1,3-benzodiazol-1-yl)pentyl]oxy}pyrazolo[1,5-a]pyridin-3-yl)-6-chloropyridine-4-carboxylate (140 mg, 0.27 mmol, 1 equiv), trimethyl-1,3,5,2,4,6-trioxatriborinane (69 mg, 0.55 mmol, 2 equiv), Pd(dppf)Cl₂—CH₂Cl₂ (3 mg, 0.01 mmol, 0.2 equiv), K₂CO₃ (76 mg, 0.55 mmol, 2 equiv) and PCy₃HBF₄ (30 mg, 0.08 mmol, 0.3 equiv) in 1,4-dioxane (10 mL) was stirred for 2 h at 100 °C under nitrogen atmosphere. The mixture was cooled to room temperature. The reaction was quenched with water (200 mL) and the mixture was extracted with ethyl acetate (3×200 mL). The combined organic layers were washed with brine (3×200 mL), dried over anhydrous Na₂SO₄, and concentrated under reduced pressure. The residue was purified on a Prep-TLC eluted with dichloromethane/methanol (20/1) to give 80 mg of methyl 2-(2-{[5-(2-amino-1,3-benzodiazol-1-yl)pentyl]oxy}pyrazolo[1,5-a]pyridin-3-yl)-6-methylpyridine-4-carboxylate (INT-B14-4) as a brown solid (59%). LCMS: m/z (EST), [M+H]⁺=485.25.

Step 6. Preparation of 2-(2-{[5-(2-amino-1,3-benzodiazol-1-yl)pentyl]oxy}pyrazolo[1,5-a]pyridin-3-yl)-6-methylpyridine-4-carboxylic acid (INT-B14-5). A mixture of methyl 2-(2-{[5-(2-amino-1,3-benzodiazol-1-yl)pentyl]oxy}pyrazolo[1,5-a]pyridin-3-yl)-6-methylpyridine-4-carboxylate (70 mg, 0.14 mmol, 1 equiv) and LiOH·H₂O (18 mg, 0.43 mmol, 3 equiv) in tetrahydrofuran (5 mL) and water (1.25 mL) was stirred for 2 h at room temperature under nitrogen atmosphere. The resulting mixture was concentrated under reduced pressure. The residue was purified by C18 FLASH using water and acetonitrile as mobile phase. Fractions containing the desired compound were evaporated to dryness to give 30 mg of 2-(2-{[5-(2-amino-1,3-benzodiazol-1-yl)pentyl]oxy}pyrazolo[1,5-a]pyridin-3-yl)-6-methylpyridine-4-carboxylic acid (INT-B14-5) as a white solid (44%). LCMS: m/z (EST), [M+H]⁺=471.30.

Step 7. Preparation of 30-methyl-11-oxa-8,9,17,24,26,31-hexaazahexacyclo[26.3.1.0\hat{ }{2,10}.0\hat{ }{3,8}.0\hat{ }{17,25}.0\hat{ }{18,23}]dotriaconta-1(31),2,4,6,9,18,20,22,24,28(32),29-undecaen-27-one (EXAMPLE B14). A mixture of 2-(2-{[5-(2-amino-1,3-benzodiazol-1-yl)pentyl]oxy}pyrazolo[1,5-a]pyridin-3-yl)-6-methylpyridine-4-carboxylic acid (25 mg, 0.05 mmol, 1 equiv), N,N-diisopropylethylamine (20 mg, 0.15 mmol, 3 equiv) and N,N,N,N-tetramethyl-O-(7-azabenzotriazol-1-yl)uronium hexafluorophospate (30 mg, 0.08 mmol, 1.5 equiv) in 1,4-dioxane (10 mL) was stirred for 2 h at room temperature under nitrogen atmosphere. The resulting mixture was concentrated under reduced pressure. The residue was purified by prep-HPLC with XBridge Prep OBD C18 column using water containing 10 mmol/L NH₄HCO₃ and 0.1% NH₃—H₂O and acetonitrile as mobile phase. Fractions containing the desired compound were evaporated to dryness to give 9.4 mg of 30-methyl-11-oxa-8,9,17,24,26,31-hexaazahexacyclo[26.3.1.0\hat{ }{2,10}.0\hat{ }{3,8}.0\hat{ }{17,25}.0\hat{ }{18,23}]dotriaconta-1(31),2,4,6,9,18,20,22,24,28(32),29-undecaen-27-one (EXAMPLE B14) as a white solid (39%). LCMS: m/z (EST), [M+H]⁺=453.20. ¹H NMR (DMSO-d₆, 400 MHz) δ 1.91-2.12 (6H, m), 2.64 (3H, s), 4.23 (2H, t), 4.48 (2H, t), 6.91 (1H, t), 7.20-7.33 (2H, m), 7.42 (1H, t), 7.49 (1H, s), 7.55 (2H, d), 8.57-8.67 (2H, m), 9.04 (1H, s), 12.66 (1H, s).

### Example B15

### 30-Chloro-11-oxa-8,9,17,24,26,31-hexaazahexacyclo[26.3.1.0\hat{ }{2,10}.0\hat{ }{3,8}.0\hat{ }{17,25}.0\hat{ }{18,23}]dotriaconta-1(31),2,4,6,9,18,20,22,24,28(32),29-undecaen-27-one

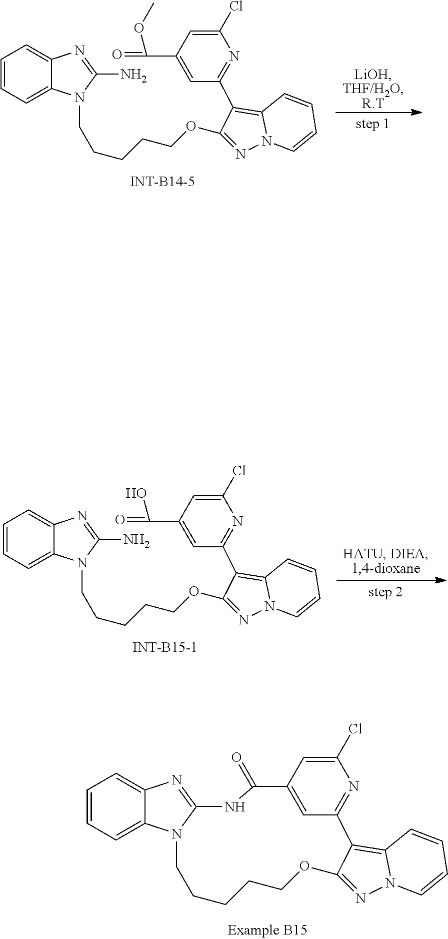

Step 1. Preparation of 2-(2-{[5-(2-amino-1,3-benzodiazol-1-yl)pentyl]oxy}pyrazolo[1,5-a]pyridin-3-yl)-6-chloropyridine-4-carboxylic acid (INT-B15-1). A mixture of methyl 2-(2-{[5-(2-amino-1,3-benzodiazol-1-yl)pentyl]oxy}pyrazolo[1,5-a]pyridin-3-yl)-6-chloropyridine-4-carboxylate (330 mg, 0.65 mmol, 1 equiv) and LiOH·H₂O (41 mg, 0.98 mmol, 1.5 equiv) in tetrahydrofuran (10 mL) and H₂O (2.5 mL) was stirred for 2 h at room temperature under nitrogen atmosphere. The resulting mixture was concentrated under reduced pressure. The residue was purified by prep-HPLC with XBridge Shield RP18 OBD column using water containing 10 mmol/L NH₄HCO₃ and acetonitrile as mobile phase. Fractions containing the desired compound were evaporated to dryness to give 134 mg of 2-(2-{[5-(2-amino-1,3-benzodiazol-1-yl)pentyl]oxy}pyrazolo[1,5-a]pyridin-3-yl)-6-chloropyridine-4-carboxylic acid (INT-B15-1) as a brown solid (41%). LCMS: m/z (ESI), [M+H]⁺=491.15.

Step 2. Preparation of 30-chloro-11-oxa-8,9,17,24,26,31-hexaazahexacyclo[26.3.1.0\hat{ }{2,10}.0\hat{ }{3,8}.0\hat{ }{17,25}.0\hat{ }{18,23}]dotriaconta-1(31),2,4,6,9,18,20,22,24,28(32),29-undecaen-27-one (EXAMPLE B15). A mixture of 2-(2-{[5-(2-amino-1,3-benzodiazol-1-yl)pentyl]oxy}pyrazolo[1,5-a]pyridin-3-yl)-6-chloropyridine-4-carboxylic acid (120 mg, 0.24 mmol, 1 equiv), N,N-diisopropylethylamine (94 mg, 0.73 mmol, 3 equiv) and N,N,N,N-tetramethyl-O-(7-azabenzotriazol-1-yl)uronium hexafluorophospate (139 mg, 0.36 mmol, 1.5 equiv) in 1,4-dioxane (10 mL) was stirred for 2 h at room temperature under nitrogen atmosphere. The reaction was quenched with water and the mixture was extracted with ethyl acetate. The combined organic layers were dried and concentrated under reduced pressure. The residue was purified by prep-HPLC with XBridge Prep OBD C18 column using water containing 10 mmol/L NH₄HCO₃ and acetonitrile as mobile phase. Fractions containing the desired compound were evaporated to dryness to give 3.9 mg of 30-chloro-11-oxa-8,9,17,24,26,31-hexaazahexacyclo[26.3.1.0\hat{ }{2,10}.0\hat{ }{3,8}.0\hat{ }{17,25}.0\hat{ }{18,23}]dotriaconta-1(31),2,4,6,9,18,20,22,25,28(32),29-undecaen-27-one (EXAMPLE B15) as a yellow solid (3%). LCMS: m/z (EST), [M+H]⁺=473.10. ¹H NMR (DMSO-d₆, 400 MHz) δ 1.93-2.13 (6H, m), 4.24 (2H, t), 4.49 (2H, t), 6.96-7.02 (1H, m), 7.22-7.35 (2H, m), 7.48-7.61 (4H, m), 8.44 (1H, d), 8.67 (1H, d), 9.17 (1H, d), 12.76 (1H, s).

### Example B16

### 5,30-Dimethyl-7-oxa-4,5,13,18,24,26,31-heptaazahexacyclo[26.3.1.0\hat{ }{2,6}.0\hat{ }{13,25}.0\hat{ }{14,23}.0\hat{ }{15,20}]dotriaconta-1(31),2(6),3,14,20,22,24,28(32),29-nonaen-27-one

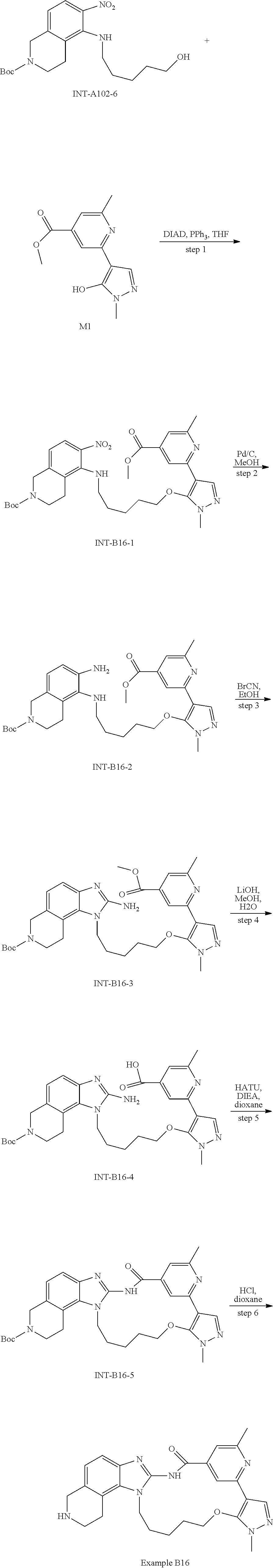

Step 1. Preparation of tert-butyl 5-((5-((4-(4-(methoxycarbonyl)-6-methylpyridin-2-yl)-1-methyl-1H-pyrazol-5-yl)oxy)pentyl)amino)-6-nitro-3,4-dihydroisoquinoline-2(1H)-carboxylate (INT-B16-1). To a stirred mixture of tert-butyl 5-[(5-hydroxypentyl)amino]-6-nitro-3,4-dihydro-1H-isoquinoline-2-carboxylate (1.6 g, 0.79 mmol, 1 equiv), triphenylphosphine (3 g, 2.37 mmol, 3 equiv) and methyl 2-(5-hydroxy-1-methylpyrazol-4-yl)-6-methylpyridine-4-carboxylate (1 g, 0.79 mmol, 1 equiv) in tetrahydrofuran (50 mL) was added diisopropyl azodicarboxylate (2.5 g, 1.19 mmol, 3 equiv) at 0 °C under nitrogen atmosphere. The resulting mixture was stirred for 3 h at room temperature under nitrogen atmosphere. The reaction was quenched with water (50 mL) and the mixture was extracted with dichloromethane (3×100 mL). The combined organic layers were washed with brine (3×100 mL), dried over anhydrous Na₂SO₄, and concentrated under reduced pressure. The residue was purified by silica gel chromatography eluted with petroleum ether/ethyl acetate (1/1) to give 900 mg of tert-butyl 5-{[5-({4-[4-(methoxycarbonyl)-6-methylpyridin-2-yl]-2-methylpyrazol-3-yl}oxy)pentyl]amino}-6-nitro-3,4-dihydro-1H-isoquinoline-2-carboxylate (INT-B16-1) as a yellow oil (36%). LCMS: m/z (ESI), [M+H]⁺=609.20.

Step 2. Preparation of tert-butyl 6-amino-5-((5-((4-(4-(methoxycarbonyl)-6-methylpyridin-2-yl)-1-methyl-1H-pyrazol-5-yl)oxy)pentyl)amino)-3,4-dihydroisoquinoline-2(1H)-carboxylate (INT-B16-2). A mixture of tert-butyl 5-{[5-({4-[4-(methoxycarbonyl)-6-methylpyridin-2-yl]-2-methylpyrazol-3-yl}oxy)pentyl]amino}-6-nitro-3,4-dihydro-1H-isoquinoline-2-carboxylate (900 mg, 1.48 mmol, 1 equiv) and Pd/C (15.73 mg, 0.148 mmol, 0.1 equiv) in methanol (10 mL) was stirred for 1 h at room temperature under hydrogen atmosphere. The resulting mixture was filtered and the filter cake was washed with methanol (3×10 mL). The resulting solution was concentrated under reduced pressure to give 570 mg of tert-butyl 6-amino-5-((5-((4-(4-(methoxycarbonyl)-6-methylpyridin-2-yl)-1-methyl-1H-pyrazol-5-yl)oxy)pentyl)amino)-3,4-dihydroisoquinoline-2(1H)-carboxylate (INT-B16-2) as a yellow oil (67%). The crude product mixture was used in the next step directly without further purification. LCMS: m/z (ESI), [M+H]⁺=579.25.

Step 3. Preparation of tert-butyl 2-amino-1-(5-((4-(4-(methoxycarbonyl)-6-methylpyridin-2-yl)-1-methyl-1H-pyrazol-5-yl)oxy)pentyl)-1,6,8,9-tetrahydro-7H-imidazo[4,5-f]isoquinoline-7-carboxylate (INT-B16-3). A mixture of tert-butyl 6-amino-5-{[5-({4-[4-(methoxycarbonyl)-6-methylpyridin-2-yl]-2-methylpyrazol-3-yl}oxy)pentyl]amino}-3,4-dihydro-1H-isoquinoline-2-carboxylate (570 mg, 0.99 mmol, 1 equiv) and BrCN (115 mg, 1.08 mmol, 1.1 equiv) in ethanol (20 mL) was stirred for 1 h at room temperature under nitrogen atmosphere. The reaction was quenched with water (20 mL) and the mixture was extracted with dichloromethane (3×50 mL). The combined organic layers were washed with brine (3×50 mL), dried over anhydrous Na₂SO₄, and concentrated under reduced pressure. The residue was purified by silica gel chromatography eluted with dichloromethane/methanol (20/1) to give 500 mg of methyl 2-[5-({5-[2-amino-7-(tert-butoxycarbonyl)-6H,8H,9H-imidazo[4,5-f]isoquinolin-1-yl]pentyl}oxy)-1-methylpyrazol-4-yl]-6-methylpyridine-4-carboxylate (INT-B16-3) as an off-white solid (84%). LCMS: m/z (ESI), [M+H]⁺=604.20.

Step 4. Preparation of 2-(5-((5-(2-amino-7-(tert-butoxycarbonyl)-6,7,8,9-tetrahydro-1H-imidazo[4,5-f]isoquinolin-1-yl)pentyl)oxy)-1-methyl-1H-pyrazol-4-yl)-6-methylisonicotinic acid (INT-B16-4). A mixture of methyl 2-[5-({5-[2-amino-7-(tert-butoxycarbonyl)-6H,8H,9H-imidazo[4,5-f]isoquinolin-1-yl]pentyl}oxy)-1-methylpyrazol-4-yl]-6-methylpyridine-4-carboxylate (500 mg, 0.828 mmol, 1 equiv) and LiOH H₂O (59.50 mg, 2.484 mmol, 3 equiv) in MeOH (10 mL) and H₂O (5 mL) was stirred for 1 h at room temperature under nitrogen atmosphere and concentrated under reduced pressure. The residue was purified by prep-HPLC with XBridge Shield RP18 OBD column using water containing 10 mmol/L NH₄HCO₃ and 0.1% NH₃·H₂O and acetonitrile as mobile phase. Fractions containing the desired compound were evaporated to dryness to give 150 mg of 2-[5-({5-[2-amino-7-(tert-butoxycarbonyl)-6H,8H,9H-imidazo[4,5-f]isoquinolin-1-yl]pentyl}oxy)-1-methylpyrazol-4-yl]pyridine-4-carboxylic acid (INT-B16-4) as a white solid (31%). LCMS: m/z (EST), [M+H]⁺=590.25.

Step 5. Preparation of tert-butyl 5,30-Dimethyl-18-(tert-butoxycarbonyl)-7-oxa-4,5,13,18,24,26,31-heptaazahexacyclo[26.3.1.0\hat{ }{2,6}.0\hat{ }{13,25}.0\hat{ }{14,23}.0\hat{ }{15,20}1]dotriaconta-1(31),2(6),3,14,20,22,24,28(32),29-nonaen-27-one (INT-B16-5). A mixture of 2-[5-({5-[2-amino-7-(tert-butoxycarbonyl)-6H,8H,9H-imidazo[4,5-f]isoquinolin-1-yl]pentyl}oxy)-1-methylpyrazol-4-yl]-6-methylpyridine-4-carboxylic acid (500 mg, 0.85 mmol, 1 equiv), N,N-diisopropylethylamine (219 mg, 1.7 mmol, 2 equiv) and N,N,N,N-tetramethyl-O-(7-azabenzotriazol-1-yl)uronium hexafluorophospate (644.8 mg, 1.7 mmol, 2 equiv) in dioxane (20 mL) was stirred for 2 h at room temperature under nitrogen atmosphere. The reaction was quenched with water (10 mL) and the mixture was extracted with dichloromethane (3×10 mL). The combined organic layers were washed with brine (3×10 mL), dried over anhydrous Na₂SO₄, and concentrated under reduced pressure. The residue was purified by silica gel column chromatography eluted with dichloromethane/methanol (10/1) to give 200 mg of 5,30-Dimethyl-18-(tert-butoxycarbonyl)-7-oxa-4,5,13,18,24,26,31-heptaazahexacyclo[26.3.1.0\hat{ }{2,6}.0\hat{ }{13,25}.0\hat{ }{14,23}.0\hat{ }{15,20}]dotriaconta-1(31),2(6),3,14,20,22,24,28(32),29-nonaen-27-one (INT-B16-5) as a brown solid (41%). LCMS: m/z (EST), [M+H]⁺=572.30. ¹H NMR (CDCl₃, 400 MHz) δ 1.64 (17H, s), 1.95 (2H, d), 2.10-2.15 (2H, m), 2.68 (3H, s), 3.24 (2H, s), 3.80 (5H, d), 4.20-4.29 (2H, m), 4.38-4.47 (2H, m), 4.72 (2H, s), 7.03 (1H, d), 7.22 (1H, d), 7.69 (1H, s), 8.21 (1H, s), 8.49 (1H, s), 12.00 (1H, s).

Step 6. Preparation of 5,30-dimethyl-7-oxa-4,5,13,18,24,26,31-heptaazahexacyclo[26.3.1.0\hat{ }{2,6}.0\hat{ }{13,25}.0\hat{ }{14,23}.0\hat{ }{15,20}]dotriaconta-1(31),2(6),3,14,20,22,24,28(32),29-nonaen-27-one (EXAMPLE B16). A mixture of tert-butyl 1¹,2⁶-dimethyl-3-oxo-5⁶,5⁷,5⁸,5⁹-tetrahydro-1¹1H,5¹H-11-oxa-4-aza-5(2,1)-imidazo[4,5-f]isoquinolina-2(2,4)-pyridina-1(4,5)-pyrazolacycloundecaphane-5⁷-carboxylate (30 mg, 0.090 mmol, 1 equiv) and HCl in 1,4-dioxane (5 mL) was stirred for 1 h at room temperature under nitrogen atmosphere. The mixture was adjusted to pH 7 with saturated NaHCO₃ (aq.) and the mixture was extracted with ethyl acetate (3×10 mL). The combined organic layers were washed with brine (3×10 mL), dried over anhydrous Na₂SO₄, and concentrated under reduced pressure. The residue was purified by prep-HPLC with XBridge Shield RP18 OBD column using water containing 10 mmol/L NH₄HCO₃ and 0.10% NH₃—H₂O and acetonitrile as mobile phase. Fractions containing the desired compound were evaporated to dryness to give 1.5 mg of 5,30-dimethyl-7-oxa-4,5,13,18,24,26,31-heptaazahexacyclo[26.3.1.0\hat{ }{2,6}.0\hat{ }{13,25}.0\hat{ }{14,23}.0\hat{ }{15,20}1]dotriaconta-1(31),2(6),3,14,20,22,24,28(32),29-nonaen-27-one (EXAMPLE B16) as a white solid (0.37%). LCMS: m/z (ESI), [M+H]⁺=472.35. ¹H NMR (DMSO, 400 MHz) δ 1.74-1.82 (2H, m), 1.96-2.10 (4H, m), 2.55 (3H, s), 3.02-3.16 (4H, m), 3.72 (3H, s), 3.95 (2H, s), 4.21 (2H, t), 4.35 (1H, d), 6.91 (1H, d), 7.33 (1H, d), 7.56 (1H, s), 7.93 (1H, s), 8.40 (1H, s).

### Example B17

### 5,30-Dimethyl-18-(oxetan-3-yl)-7-oxa-4,5,13,18,24,26,31-heptaazahexacyclo[26.3.1.0\hat{ }{2,6}.0\hat{ }{13,25}.0\hat{ }{14,23}.0\hat{ }{15,20}]dotriaconta-1(31),2(6),3,14,20,22,24,28(32),29-nonaen-27-one

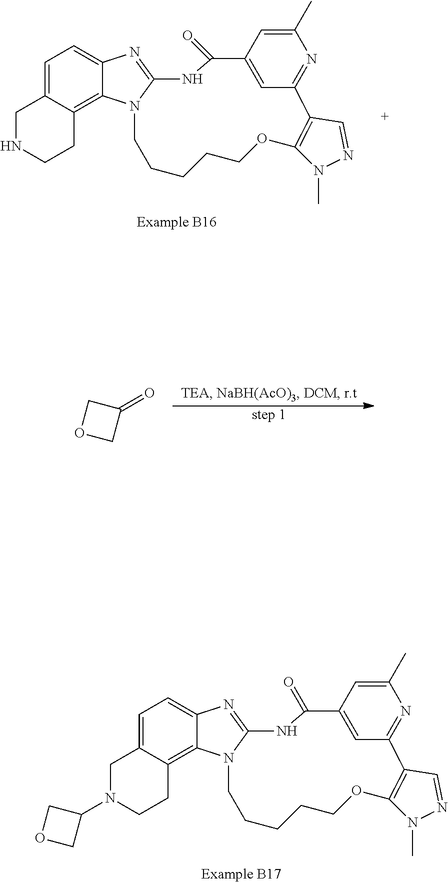 [SMILES: Cc1cc2cc(n1)-c1cnn(C)c1OCCCCCn1c(nc3ccc4c(c31)CCNC4)NC2=O ; O=C1COC1 ; "TEA, NaBH(AcO)3, DCM, r.t" ; "step 1" ; Cc1cc2cc(n1)-c1cnn(C)c1OCCCCCn1c(nc3ccc4c(c31)CCN(C1COC1)C4)NC2=O]

Step 1. Preparation of 5,30-dimethyl-18-(oxetan-3-yl)-7-oxa-4,5,13,18,24,26,31-heptaazahexacyclo[26.3.1.0\hat{ }{2,6}.0\hat{ }{13,25}.0\hat{ }{14,23}.0\hat{ }{15,20}]dotriaconta-1(31),2(6),3,14,20,22,24,28(32),29-nonaen-27-one (EXAMPLE B17). To a stirred mixture of (25E)-5,30-dimethyl-7-oxa-4,5,13,18,24,26,31-heptaazahexacyclo[26.3.1.0\hat{ }{2,6}.0\hat{ }{13,25}.0\hat{ }{14,23}.0\hat{ }{15,20}]dotriaconta-1(31),2(6),3,14,20,22,25,28(32),29-nonaen-27-one (EXAMPLE B16, 30 mg, 0.06 mmol, 1 equiv), 3-oxetanone (9.17 mg, 0.12 mmol, 2 equiv) and trimethylamine (12.88 mg, 0.12 mmol, 2 equiv) in dichloromethane (5 mL) was stirred for 30 min at room temperature under nitrogen atmosphere. To the above mixture was added NaBH(AcO)₃ (40.45 mg, 0.18 mmol, 3 equiv) at room temperature. The resulting mixture was stirred overnight at room temperature under nitrogen atmosphere. The reaction was quenched with water (10 mL) and the mixture was extracted with dichloromethane (3×10 mL). The combined organic layers were washed with brine (3×10 mL), dried over anhydrous Na₂SO₄, and concentrated under reduced pressure. The residue was purified by prep-HPLC with XBridge Shield RP18 OBD column using water containing 10 mmol/L NH₄HCO₃ and 0.10% NH₃·H₂O and acetonitrile as mobile phase. Fractions containing the desired compound were evaporated to dryness to give 5.8 mg of 5,30-dimethyl-18-(oxetan-3-yl)-7-oxa-4,5,13,18,24,26,31-heptaazahexacyclo[26.3.1.0\hat{ }{2,6}.0\hat{ }{13,25}.0\hat{ }{14,23}.0\hat{ }{15,20}]dotriaconta-1(31),2(6),3,14,20,22,24,28(32),29-nonaen-27-one (EXAMPLE B17) as a yellow solid (17%). LCMS: m/z (ESI), [M+H]⁺=528.20. ¹H NMR (DMSO-d₆, 400 MHz) δ 1.71-1.85 (2H, m), 1.96-2.13 (4H, m), 2.56 (3H, s), 2.59-2.70 (2H, m), 3.22-3.30 (2H, m), 3.54 (2H, s), 3.57-3.65 (1H, m), 3.73 (3H, s), 4.21 (1H, t), 4.33 (2H, t), 4.55 (2H, t), 4.65 (2H, t), 6.96 (1H, d), 7.46 (1H, d), 7.57 (1H, s), 7.76 (1H, d), 7.98 (1H, s), 8.40 (1H, s).

### Example B18

### 5,30-dimethyl-17-(oxetan-3-yl)-7-oxa-4,5,13,17,24,26,31-heptaazahexacyclo[26.3.1.0\hat{ }{2,6}.0\hat{ }{13,25}.0\hat{ }{14,23}.0\hat{ }{15,20}]dotriaconta-1(31),2(6),3,14,20,22,24,28(32),29-nonaen-27-one

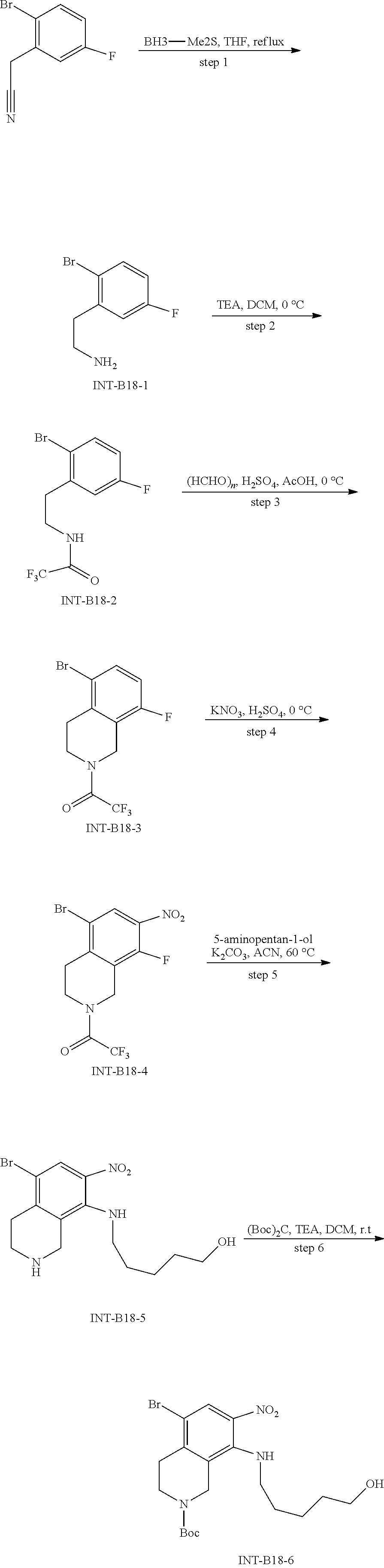

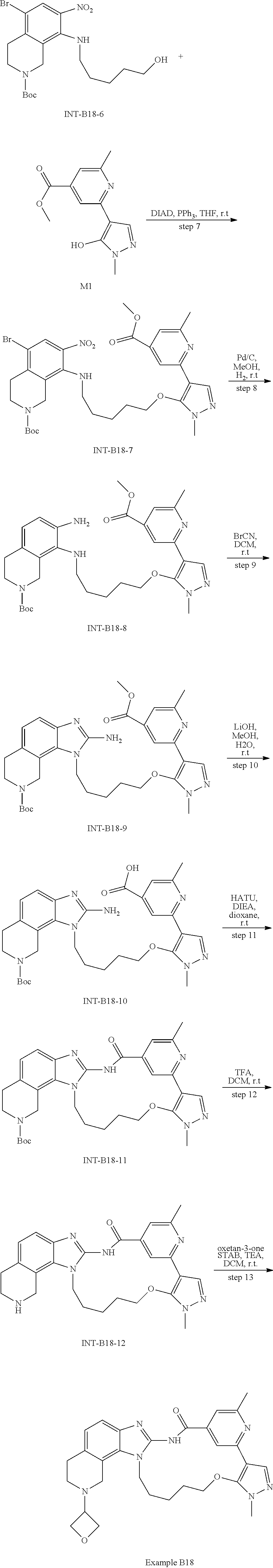

Step 1. Preparation of 2-(2-bromo-5-fluorophenyl)ethanamine (INT-B18-1). A mixture of 2-(2-bromo-5-fluorophenyl)acetonitrile (10 g, 46.72 mmol, 1 equiv) and BH₃-Me₂S (10.65 g, 140.16 mmol, 3 equiv) in tetrahydrofuran (100 mL) was stirred for 2 h at 80 °C under nitrogen atmosphere. The mixture was cooled to room temperature. The reaction was quenched with water (800 mL) at 0 °C and the mixture was extracted with dichloromethane (3×200 mL). The combined organic layers were washed with brine (3×200 mL), dried over anhydrous Na₂SO₄, and concentrated under reduced pressure. The residue was purified by silica gel chromatography eluted with dichloromethane/methanol (10/1) to give 8 g of 2-(2-bromo-5-fluorophenyl)ethanamine (INT-B18-1) as an off-white solid (79%). LCMS: m/z (ESI), [M+H]⁺=220.10.

Step 2. Preparation of N-[2-(2-bromo-5-fluorophenyl)ethyl]-2,2,2-trifluoroacetamide (INT-B18-2). A mixture of 2-(2-bromo-5-fluorophenyl)ethanamine (5 g, 23 mmol, 1 equiv), trifluoroacetic anhydride (5.3 g, 25 mmol, 1.1 equiv) and trimethylamine (4.64 g, 45.86 mmol, 2 equiv) in dichloromethane (100 mL) was stirred for 2 h at 0 °C under nitrogen atmosphere. The reaction was quenched with water (200 mL) and the mixture was extracted with dichloromethane (3×200 mL). The combined organic layers were washed with brine (3×200 mL), dried over anhydrous Na₂SO₄ and concentrated under reduced pressure. The residue was purified by silica gel column chromatography eluted with petroleum ether/ethyl acetate (10/1) to give 4 g of N-[2-(2-bromo-5-fluorophenyl)ethyl]-2,2,2-trifluoroacetamide (INT-B18-2) as a white solid (56%). ¹H NMR (DMSO-d₆, 400 MHz) δ 2.90-2.96 (2H, m), 3.45-3.50 (2H, m), 7.04-7.26 (2H, m), 7.65 (1H, d), 9.55 (1H, s).

Step 3. Preparation of 1-(5-bromo-8-fluoro-3,4-dihydro-1H-isoquinolin-2-yl)-2,2,2-trifluoroethanone (INT-B18-3). A mixture of N-[2-(2-bromo-5-fluorophenyl)ethyl]-2,2,2-trifluoroacetamide (6.8 g, 21.65 mmol, 1 equiv) and paraformaldehyde (28.6 g, 64.95 mmol, 3 equiv) in H₂SO₄ (25 ml) and AcOH (30 ml) was stirred for overnight at 0 °C under nitrogen atmosphere. The reaction was quenched with water (200 mL) at 0 °C and adjusted to pH 7 with NaOH. The mixture was extracted with dichloromethane (3×200 mL). The combined organic layers were washed with brine (3×200 mL), dried over anhydrous Na₂SO₄, and concentrated under reduced pressure. The residue was purified by silica gel chromatography eluted with petroleum ether/ethyl acetate (1/1) to give 5 g of 1-(5-bromo-8-fluoro-3,4-dihydro-1H-isoquinolin-2-yl)-2,2,2-trifluoroethanone as an off-white solid (710%). ¹H NMR (DMSO-d₆, 400 MHz) δ 2.85-2.92 (2H, m), 3.85-3.95 (2H, m), 4.77 (2H, d), 7.17 (1H, d), 7.63 (1H, d).

Step 4. Preparation of 1-(5-bromo-8-fluoro-7-nitro-3,4-dihydro-1H-isoquinolin-2-yl)-2,2,2-trifluoroethanone (INT-B18-4). A mixture of 1-(5-bromo-8-fluoro-3,4-dihydro-1H-isoquinolin-2-yl)-2,2,2-trifluoroethanone (6.46 g, 19.81 mmol, 1 equiv) and KNO₃ (2 g, 19.81 mmol, 1 equiv) in H₂SO₄ (20 mL) was stirred for 0.5 h at 0 °C under nitrogen atmosphere. The reaction was quenched with water (200 mL) and adjusted to pH 9 with NaOH. The mixture was extracted with dichloromethane (3×200 mL). The combined organic layers were washed with brine (3×200 mL), dried over anhydrous Na₂SO₄, and concentrated under reduced pressure. The residue was purified by silica gel column chromatography eluted with petroleum ether/ethyl acetate (3/1) to give 775 mg of 1-(5-bromo-8-fluoro-7-nitro-3,4-dihydro-1H-isoquinolin-2-yl)-2,2,2-trifluoroethanone (INT-B18-4) as a brown yellow solid (12%). ¹H NMR (DMSO-dd, 400 MHz) δ 2.95-3.02 (2H, m), 3.81-3.96 (2H, m), 4.75-4.90 (2H, m), 8.07 (1H, t).

Step 5. Preparation of 5-[(5-bromo-7-nitro-1,2,3,4-tetrahydroisoquinolin-8-yl)amino]pentan-1-ol (INT-B18-5). A mixture of 1-(5-bromo-8-fluoro-7-nitro-3,4-dihydro-1H-isoquinolin-2-yl)-2,2,2-trifluoroethanone (4.6 g, 12.4 mmol, 1 equiv), K₂CO₃ (5.14 g, 37.2 mmol, 3 equiv) and 5-aminopentanol (1.53 g, 14.88 mmol, 1.2 equiv) in acetonitrile (50 mL) was stirred for 2 h at 60 °C under nitrogen atmosphere. The mixture was cooled to room temperature. The reaction was quenched with water (100 mL) and the mixture was extracted with ethyl acetate (3×100 mL). The combined organic layers were washed with brine (3×100 mL), dried over anhydrous Na₂SO₄, and concentrated under reduced pressure. The residue was purified by silica gel column chromatography eluted with dichloromethane/methanol (10/1) to give 600 mg of 5-[(5-bromo-7-nitro-1,2,3,4-tetrahydroisoquinolin-8-yl)amino]pentan-1-ol (INT-B18-5) as a yellow oil (14%). LCMS: m/z (ESI), [M+H]⁺=360.00.

Step 6. Preparation of tert-butyl 5-bromo-8-((5-hydroxypentyl)amino)-7-nitro-3,4-dihydroisoquinoline-2(1H)-carboxylate (INT-B18-6). A mixture of 5-[(5-bromo-7-nitro-1,2,3,4-tetrahydroisoquinolin-8-yl)amino]pentan-1-ol (650 mg, 1.81 mmol, 1 equiv), di-tert-butyl dicarbonate (475 mg, 2.18 mmol, 1.2 equiv), and trimethylamine (367 mg, 3.63 mmol, 2 equiv) in dichloromethane (20 mL) was stirred overnight at room temperature under nitrogen atmosphere. The reaction mixture was concentrated under reduced pressure. The residue was purified by C18 FLASH using water and acetonitrile as mobile phase. Fractions containing the desired compound were evaporated to dryness to give 450 mg of tert-butyl 5-bromo-8-((5-hydroxypentyl)amino)-7-nitro-3,4-dihydroisoquinoline-2(1H)-carboxylate (INT-B18-6) as a yellow oil (54%). LCMS: m/z (ESI), [M+H]⁺=460.25.

Step 7. Preparation of tert-butyl 5-bromo-8-{[5-({4-[4-(methoxycarbonyl)-6-methylpyridin-2-yl]-2-methylpyrazol-3-yl}oxy)pentyl]amino}-7-nitro-3,4-dihydro-1H-isoquinoline-2-carboxylate (INT-B18-7). To a stirred mixture of tert-butyl 5-bromo-8-((5-hydroxypentyl)amino)-7-nitro-3,4-dihydroisoquinoline-2(1H)-carboxylate (450 mg, 0.98 mmol, 1 equiv), triphenylphosphine (772 mg, 2.95 mmol, 3 equiv) in tetrahydrofuran (20 mL) were added methyl 2-(5-hydroxy-1-methylpyrazol-4-yl)-6-methylpyridine-4-carboxylate (267 mg, 1.08 mmol, 1.1 equiv) and diisopropyl azodicarboxylate (595 mg, 2.95 mmol, 3 equiv) at 0 °C under nitrogen atmosphere. The reaction mixture was stirred for 3 h at 60 °C under nitrogen atmosphere and cooled to room temperature. The reaction was quenched with water (100 mL) and the mixture was extracted with ethyl acetate (3×100 mL). The combined organic layers were washed with brine (3×100 mL), dried over anhydrous Na₂SO₄, and concentrated under reduced pressure. The residue was purified by C18 FLASH using water and acetonitrile as mobile phase. Fractions containing the desired compound were evaporated to dryness to give 350 mg of tert-butyl 5-bromo-8-{[5-({4-[4-(methoxycarbonyl)-6-methylpyridin-2-yl]-2-methylpyrazol-3-yl}oxy)pentyl]amino}-7-nitro-3,4-dihydro-1H-isoquinoline-2-carboxylate (INT-B18-7) as a yellow solid (52%). LCMS: m/z (ESI), [M+H]⁺=689.05.

Step 8. Preparation of tert-butyl 7-amino-8-((5-((4-(4-(methoxycarbonyl)-6-methylpyridin-2-yl)-1-methyl-1H-pyrazol-5-yl)oxy)pentyl)amino)-3,4-dihydroisoquinoline-2(1H)-carboxylate (INT-B18-8). A mixture of tert-butyl 5-bromo-8-{[5-({4-[4-(methoxycarbonyl)-6-methylpyridin-2-yl]-2-methylpyrazol-3-yl}oxy)pentyl]amino}-7-nitro-3,4-dihydro-1H-isoquinoline-2-carboxylate (330 mg, 0.48 mmol, 1 equiv) and Pd/C (5.1 mg, 0.048 mmol, 0.1 equiv) in methanol (20 mL) was stirred for 1 h at room temperature under hydrogen atmosphere. The resulting mixture was filtered and the filter cake was washed with methanol (3×30 mL). The resulting solution was concentrated under reduced pressure to give 172 mg of tert-butyl 7-amino-8-((5-((4-(4-(methoxycarbonyl)-6-methylpyridin-2-yl)-1-methyl-1H-pyrazol-5-yl)oxy)pentyl)amino)-3,4-dihydroisoquinoline-2(1H)-carboxylate (INT-B18-8) as a yellow oil. This material was used for the next reaction without further purification. LCMS: m/z (ESI), [M+H]⁺=579.25.

Step 9. Preparation of methyl 2-[5-({5-[2-amino-5-bromo-8-(tert-butoxycarbonyl)-6H,7H,9H-imidazo[4,5-h]isoquinolin-1-yl]pentyl}oxy)-1-methylpyrazol-4-yl]-6-methylpyridine-4-carboxylate (INT-B18-9). A mixture of tert-butyl 7-amino-8-((5-((4-(4-(methoxycarbonyl)-6-methylpyridin-2-yl)-1-methyl-1H-pyrazol-5-yl)oxy)pentyl)amino)-3,4-dihydroisoquinoline-2(1H)-carboxylate (135 mg, ~0.21 mmol, ~1 equiv) and BrCN (33 mg, 0.31 mmol, 1.5 equiv) in dichloromethane (5 mL) was stirred overnight at room temperature under nitrogen atmosphere. The reaction was quenched with water (20 mL) and the mixture was extracted with dichloromethane (3×50 mL). The combined organic layers were washed with brine (3×50 mL), dried over anhydrous Na₂SO₄, and concentrated under reduced pressure. The residue was purified by silica gel column chromatography and eluted with dichloromethane/methanol (10/1) to give 90 mg of 2-[5-({5-[2-amino-5-bromo-8-(tert-butoxycarbonyl)-6H,7H,9H-imidazo[4,5-h]isoquinolin-1-yl]pentyl}oxy)-1-methylpyrazol-4-yl]-6-methylpyridine-4-carboxylate (INT-B18-9) as a light yellow oil (64%). LCMS: m/z (ESI), [M+H]⁺=604.25.

Step 10. Preparation of 2-[5-({5-[2-amino-8-(tert-butoxycarbonyl)-6H,7H,9H-imidazo[4,5-h]isoquinolin-1-yl]pentyl}oxy)-1-methylpyrazol-4-yl]-6-methylpyridine-4-carboxylic acid (INT-B18-10). A mixture of 2-[5-({5-[2-amino-5-bromo-8-(tert-butoxycarbonyl)-6H,7H,9H-imidazo[4,5-h]isoquinolin-1-yl]pentyl}oxy)-1-methylpyrazol-4-yl]-6-methylpyridine-4-carboxylate (90 mg, 0.15 mmol, 1 equiv) and LiOH H₂O (19 mg, 0.45 mmol, 3 equiv) in MeOH (2 ml) and H₂O (0.5 ml) was stirred for 2 h at room temperature under nitrogen atmosphere. The reaction mixture was concentrated under reduced pressure. The residue was purified by C18 FLASH using water and acetonitrile as mobile phase. Fractions containing the desired compound were evaporated to dryness to give 50 mg of 2-[5-({5-[2-amino-8-(tert-butoxycarbonyl)-6H,7H,9H-imidazo[4,5-h]isoquinolin-1-yl]pentyl}oxy)-1-methylpyrazol-4-yl]-6-methylpyridine-4-carboxylic acid (INT-B18-10) as a white solid (57%). LCMS: m/z (ESI), [M+H]⁺=590.50.

Step 11. Preparation of tert-butyl 5,30-dimethyl-27-oxo-7-oxa-4,5,13,17,24,26,31-heptaazahexacyclo[26.3.1.0\hat{ }{2,6}.0\hat{ }{13,25}.0\hat{ }{14,23}.0\hat{ }{15,20}]dotriaconta-1(31),2(6),3,14,20,22,24,28(32),29-nonaene-17-carboxylate (INT-B18-11). A mixture of 2-[5-({5-[2-amino-8-(tert-butoxycarbonyl)-6H,7H,9H-imidazo[4,5-h]isoquinolin-1-yl]pentyl}oxy)-1-methylpyrazol-4-yl]-6-methylpyridine-4-carboxylic acid (35 mg, 0.05 mmol, 1 equiv), N,N-diisopropylethylamine (19.26 mg, 0.15 mmol, 3 equiv) and N,N,N,N-tetramethyl-O-(7-azabenzotriazol-1-yl)uronium hexafluorophospate (58.03 mg, 0.15 mmol, 3 equiv) in dioxane (2 mL) was stirred for 2 h at room temperature under nitrogen atmosphere. The reaction was quenched with water (10 mL) and the mixture was extracted with dichloromethane (3×10 mL). The combined organic layers were washed with brine (3×10 mL), dried over anhydrous Na₂SO₄, and concentrated under reduced pressure. The residue was purified by silica gel column chromatography and eluted with dichloromethane/methanol (10/1) to give 30 mg of tert-butyl 5,30-dimethyl-27-oxo-7-oxa-4,5,13,17,24,26,31-heptaazahexacyclo[26.3.1.0\hat{ }{2,6}.0\hat{ }{13,25}.0\hat{ }{14,23}.0\hat{ }{15,20}1]dotriaconta-1(31),2(6),3,14,20,22,24,28(32),29-nonaene-17-carboxylate (INT-B18-11) as a white solid (93%). LCMS: m/z (EST), [M+H]⁺=670.55.

Step 12. Preparation of 5,30-dimethyl-7-oxa-4,5,13,17,24,26,31-heptaazahexacyclo[26.3.1.0\hat{ }{2,6}.0\hat{ }{13,25}.0\hat{ }{14,23}.0\hat{ }{15,20}]dotriaconta-1(31),2(6),3,14,20,22,24,28(32),29-nonaen-27-one (INT-B18-12). A mixture of tert-butyl 5,30-dimethyl-27-oxo-7-oxa-4,5,13,17,24,26,31-heptaazahexacyclo[26.3.1.0\hat{ }{2,6}.0\hat{ }{13,25}.0\hat{ }{14,23}.0\hat{ }{15,20}]dotriaconta-1(31),2(6),3,14,20,22,24,28(32),29-nonaene-17-carboxylate (30 mg, 0.05 mmol, 1 equiv) in trifluoroacetic acid (1 mL) and dichloromethane (2 mL) was stirred for 30 min at room temperature. The resulting mixture was concentrated under reduced pressure. The residue was purified by prep-HPLC with XBridge Shield RP18 OBD column using water containing 10 mmol/L NH₄HCO₃ and 0.1% NH₃—H₂O and acetonitrile as mobile phase. Fractions containing the desired compound were evaporated to dryness to give 2 mg of 5,30-dimethyl-7-oxa-4,5,13,17,24,26,31-heptaazahexacyclo[26.3.1.0\hat{ }{2,6}.0\hat{ }{13,25}.0\hat{ }{14,23}.0\hat{ }{15,20}1]dotriaconta-1(31),2(6),3,14,20,22,24,28(32),29-nonaen-27-one (INT-B18-12) as a white solid (8%). LCMS: m/z (EST), [M+H]⁺=472.15. ¹H NMR (DMSO-d₆, 400 MHz) δ 1.79 (2H, s), 2.04 (4H, s), 2.56 (3H, s), 2.80 (2H, d), 2.97 (2H, t), 3.73 (3H, s), 4.20-4.29 (4H, m), 4.32 (2H, s), 6.95 (1H, d), 7.34 (1H, d), 7.57 (1H, d), 7.92 (1H, s), 8.39 (1H, s).

Step 13. Preparation of 5,30-dimethyl-17-(oxetan-3-yl)-7-oxa-4,5,13,17,24,26,31-heptaazahexacyclo[26.3.1.0\hat{ }{2,6}.0\hat{ }{13,25}.0\hat{ }{14,23}.0\hat{ }{15,20}]dotriaconta-1(31),2(6),3,14,20,22,24,28(32),29-nonaen-27-one (EXAMPLE B18). A mixture of 5,30-dimethyl-7-oxa-4,5,13,17,24,26,31-heptaazahexacyclo[26.3.1.0\hat{ }{2,6}.0\hat{ }{13,25}.0\hat{ }{14,23}.0\hat{ }{15,20}]dotriaconta-1(31),2(6),3,14,20,22,24,28(32),29-nonaen-27-one (7 mg, 0.015 mmol, 1 equiv), 3-oxetanone (1.28 mg, 0.018 mmol, 1.2 equiv) and trimethylamine (3 mg, 0.030 mmol, 2 equiv) in dichloromethane (2 mL) was stirred for 30 min at room temperature under nitrogen atmosphere. To the above mixture was added sodium triacetoxyborohydride (9.44 mg, 0.045 mmol, 3 equiv) in portions at room temperature. The resulting mixture was stirred overnight at room temperature under nitrogen atmosphere. The reaction was quenched with water (10 mL) and the mixture was extracted with dichloromethane (3×10 mL). The combined organic layers were washed with brine (3×10 mL), dried over anhydrous Na₂SO₄, and concentrated under reduced pressure. The resulting mixture was concentrated under reduced pressure. The residue was purified by prep-HPLC with XBridge Shield RP18 OBD column using water containing 10 mmol/L NH₄HCO₃ and 0.1% NH₃·H₂O and acetonitrile as mobile phase. Fractions containing the desired compound were evaporated to dryness to give 1.8 mg of 5,30-dimethyl-17-(oxetan-3-yl)-7-oxa-4,5,13,17,24,26,31-heptaazahexacyclo[26.3.1.0\hat{ }{2,6}.0\hat{ }{13,25}.0\hat{ }{14,23}.0\hat{ }{15,20}]dotriaconta-1(31),2(6),3,14,20,22,24,28(32),29-nonaen-27-one (EXAMPLE B18) as a white solid (23%). LCMS: m/z (EST), [M+H]⁺=528.20. ¹H NMR (DMSO-d₆, 400 MHz) δ 1.66-1.80 (2H, m), 1.86-2.11 (4H, m), 2.56 (3H, s), 2.87-2.98 (4H, m), 3.17 (2H, d), 3.73 (3H, s), 3.75-3.81 (1H, m), 4.04 (4H, s), 4.16-4.28 (4H, m), 4.61 (2H, t), 4.70 (2H, t), 7.01 (1H, d), 7.38 (1H, d), 7.57 (1H, s), 7.93 (1H, s), 8.40 (1H, s).

### Example B20

### 5,16,26-Trimethyl-7-oxa-4,5,13,16,20,22,27-heptaazapentacyclo[22.3.1.0\hat{ }{2,6}.0\hat{ }{13,21}.0\hat{ }{14,19}]octacosa-1(27),2(6),3,14(19),17,20,24(28),25-octaene-15,23-dione

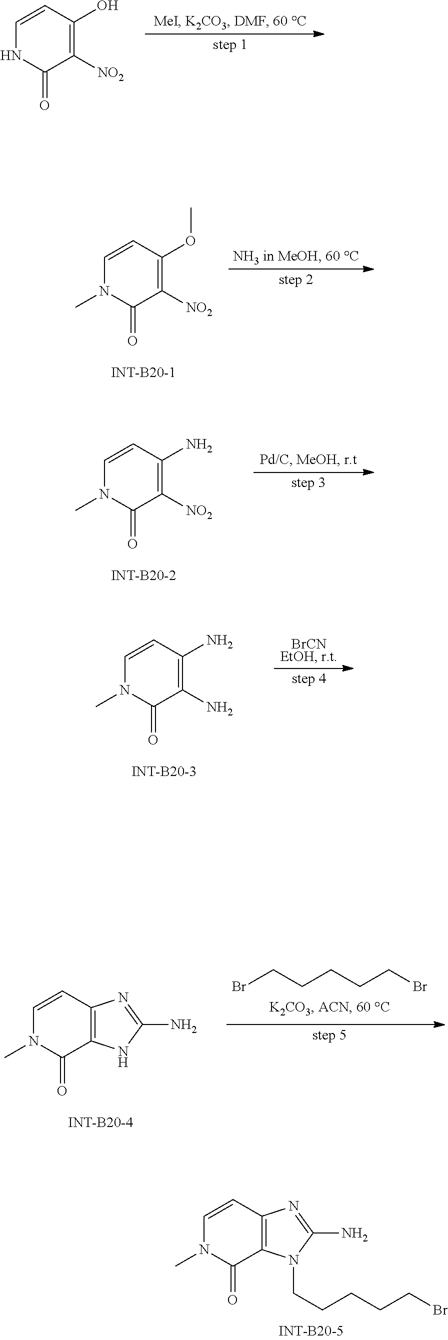

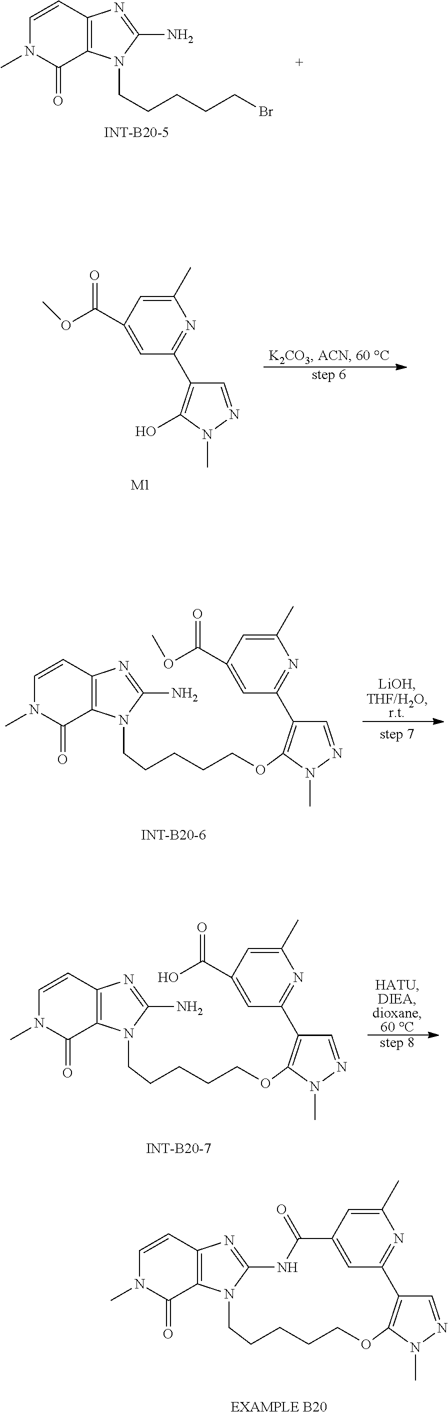

Step 1. Preparation of 4-methoxy-1-methyl-3-nitropyridin-2-one (INT-B20-1). A mixture of 4-hydroxy-3-nitro-1H-pyridin-2-one (10 g, 64.0 mmol, 1 equiv), iodomethane (36.4 g, 256.2 mmol, 4 equiv) and K₂CO₃ (35.4 g, 256.2 mmol, 4 equiv) in N,N-dimethylformamide (100 mL) was stirred overnight at 60 °C under nitrogen atmosphere and cooled to room temperature. The reaction was quenched with water (500 mL) and the mixture was extracted with dichloromethane (3×500 mL). The combined organic layers were washed with brine (3×500 mL), dried over anhydrous Na₂SO₄, and concentrated under reduced pressure. The residue was purified by C18 FLASH using water and acetonitrile as mobile phase. Fractions containing the desired compound were evaporated to dryness to give 1600 mg of 4-methoxy-1-methyl-3-nitropyridin-2-one (INT-B20-1) as a yellow solid (13%). LCMS: m/z (EST), [M+H]⁺=185.15. ¹H NMR (DMSO-d₆, 400 MHz) δ 3.48 (3H, s), 3.96 (3H, s), 6.50 (1H, d), 8.03 (1H, d).

Step 2. Preparation of 4-amino-1-methyl-3-nitropyridin-2-one (INT-B20-2). A mixture of 4-methoxy-1-methyl-3-nitropyridin-2-one (800 mg, 4.34 mmol, 1 equiv) and NH₃ in methanol (20 mL) was stirred for 4 h at room temperature under nitrogen atmosphere. The precipitated solids were collected by filtration and washed with methanol (3×40 mL) to give 630 mg of 4-amino-1-methyl-3-nitropyridin-2-one (INT-B20-2) as a yellow solid (85%). LCMS: m/z (EST), [M+H]⁺=170.05. ¹H NMR (DMSO-d₆, 400 MHz) δ 3.28 (3H, s), 5.87 (1H, d), 7.55 (1H, d), 8.03 (2H, s).

Step 3. Preparation of 3,4-diamino-1-methylpyridin-2-one (INT-B20-3). A mixture of 4-amino-1-methyl-3-nitropyridin-2-one (1 g, 5.91 mmol, 1 equiv) and Pd/C (0.31 g, 2.91 mmol, 0.49 equiv) in methanol (30 mL) was stirred for 4 h at room temperature under hydrogen atmosphere. The resulting mixture was filtered and the filter cake was washed with methanol (3×40 mL). The organic solution was concentrated under reduced pressure to give 900 mg of 3,4-diamino-1-methylpyridin-2-one (INT-B20-3) as a black solid. This material was used for the next reaction without further purification. LCMS: m/z (ESI), [M+H]⁺=140.10.

Step 4. Preparation of 2-amino-5-methyl-3H-imidazo[4,5-c]pyridin-4-one (INT-B20-4). A mixture of 3,4-diamino-1-methylpyridin-2-one (INT-B20-3, 800 mg, 5.74 mmol, 1 equiv) and BrCN (730.71 mg, 6.89 mmol, 1.20 equiv) in ethanol (15 mL) was stirred for 3 h at room temperature under nitrogen atmosphere. The resulting mixture was concentrated under vacuum. The crude product mixture was used in the next step directly without further purification. LCMS: m/z (ESI), [M+H]⁺=165.15.

Step 5. Preparation of 2-amino-3-(5-bromopentyl)-5-methylimidazo[4,5-c]pyridin-4-one (INT-B20-5). A mixture of 2-amino-5-methyl-3H-imidazo[4,5-c]pyridin-4-one (INT-B20-4, 0.8 g, 4.87 mmol, 1 equiv), 1,5-dibromopentane (5.6 g, 24.36 mmol, 5 equiv), and K₂CO₃ (1.68 g, 12.13 mmol, 2.49 equiv) in acetonitrile (15 mL) was stirred for 2 h at 60 °C under nitrogen atmosphere and cooled to room temperature. The reaction was quenched with water (100 mL) and the mixture was extracted with ethyl acetate (3×60 mL). The combined organic layers were washed with brine (3×50 mL), dried over anhydrous Na₂SO₄, and concentrated under reduced pressure. The residue was purified by Prep-TLC eluted with ethyl acetate to give 230 mg of 2-amino-3-(5-bromopentyl)-5-methylimidazo[4,5-c]pyridin-4-one (INT-B20-5) as a white solid (15%). LCMS: m/z (ESI), [M+H]⁺=313.10. ¹H NMR (DMSO-d₆, 400 MHz) δ 1.38-1.52 (2H, m), 1.53-1.65 (2H, m), 1.74-1.85 (2H, m), 3.07-3.20 (2H, m), 3.28 (3H, s), 3.48-3.56 (2H, m), 5.76 (1H, d), 6.39 (2H, s), 7.34 (1H, d).

Step 6. Preparation of methyl 2-{5-[(5-{2-amino-5-methyl-4-oxoimidazo[4,5-c]pyridin-3-yl}pentyl)oxy]-1-methylpyrazol-4-yl}-6-methylpyridine-4-carboxylate (INT-B20-6). A mixture of 2-amino-3-(5-bromopentyl)-5-methylimidazo[4,5-c]pyridin-4-one (350 mg, 1.11 mmol, 1 equiv), methyl 2-(5-hydroxy-1-methylpyrazol-4-yl)-6-methylpyridine-4-carboxylate (M1, 303 mg, 1.23 mmol, 1.10 equiv), and K₂CO₃ (386 mg, 2.79 mmol, 2.5 equiv) in acetonitrile (5 mL) was stirred for 4 h at 60 °C under nitrogen atmosphere and then cooled to room temperature. The reaction was quenched with water (100 mL) and the mixture was extracted with dichloromethane (3×60 mL). The combined organic layers were washed with brine (3×50 mL), dried over anhydrous Na₂SO₄, and concentrated under reduced pressure. The residue was purified by Prep-TLC eluted with dichloromethane/methanol (15/1) to give 160 mg of methyl 2-{5-[(5-{2-amino-5-methyl-4-oxoimidazo[4,5-c]pyridin-3-yl}pentyl)oxy]-1-methylpyrazol-4-yl}-6-methylpyridine-4-carboxylate (INT-B20-6) as a yellow solid (29%). LCMS: m/z (ESI), [M+H]⁺=480.15.

Step 7. Preparation of 2-{5-[(5-{2-amino-5-methyl-4-oxoimidazo[4,5-c]pyridin-3-yl}pentyl)oxy]-1-methylpyrazol-4-yl}-6-methylpyridine-4-carboxylic acid (INT-B20-7). A mixture of methyl 2-{5-[(5-{2-amino-5-methyl-4-oxoimidazo[4,5-c]pyridin-3-yl}pentyl)oxy]-1-methylpyrazol-4-yl}-6-methylpyridine-4-carboxylate (160 mg, 0.33 mmol, 1 equiv) and LiOH·H₂O (28 mg, 0.66 mmol, 2 equiv) in THF (1 mL) and H₂O (0.25 mL) was stirred for 2 h at room temperature. The resulting mixture was concentrated under reduced pressure. The residue was purified by reverse flash chromatography eluted with acetonitrile and water to give 130 mg of 2-{5-[(5-{2-amino-5-methyl-4-oxoimidazo[4,5-c]pyridin-3-yl}pentyl)oxy]-1-methylpyrazol-4-yl}-6-methylpyridine-4-carboxylic acid (INT-B20-7) as a white solid (83%). LCMS: m/z (ESI), [M+H]⁺=466.10.

Step 8. Preparation of 5,16,26-trimethyl-7-oxa-4,5,13,16,20,22,27-heptaazapentacyclo[22.3.1.0\hat{ }{2,6}.0\hat{ }{13,21}.0\hat{ }{14,19}]octacosa-1(27),2(6),3,14(19),17,20,24(28),25-octaene-15,23-dione (EXAMPLE B20). A mixture of 2-{5-[(5-{2-amino-5-methyl-4-oxoimidazo[4,5-c]pyridin-3-yl}pentyl)oxy]-1-methylpyrazol-4-yl}-6-methylpyridine-4-carboxylic acid (105 mg, 0.22 mmol, 1 equiv), N,N,N,N-tetramethyl-O-(7-azabenzotriazol-1-yl)uroniumhexafluorophospate (128 mg, 0.33 mmol, 1.5 equiv) and N,N-diisopropylethylamine (87 mg, 0.67 mmol, 3 equiv) in 1,4-dioxane (3 mL) was stirred overnight at room temperature under nitrogen atmosphere. The reaction was quenched with water (60 mL) and the mixture was extracted with dichloromethane (3×40 mL). The combined organic layers were washed with brine (3×30 mL), dried over anhydrous Na₂SO₄, and concentrated under reduced pressure. The residue was purified by trituration with DMF/DMSO (5 mL) to give 15 mg of 5,16,26-trimethyl-7-oxa-4,5,13,16,20,22,27-heptaazapentacyclo[22.3.1.0\hat{ }{2,6}.0\hat{ }{13,21}.0\hat{ }{14,19}]octacosa-1(27),2(6),3,14(19),17,20,24(28),25-octaene-15,23-dione (EXAMPLE B20) as a yellow solid (13%). LCMS: m/z (EST), [M+H]⁺=448.10. ¹H NMR (DMSO-d₆, 400 MHz) δ 1.66-1.80 (2H, m), 1.86-2.11 (4H, m), 2.55 (3H, s), 3.53 (3H, s), 3.73 (3H, s), 4.40 (2H, t), 4.49 (2H, t), 6.58 (1H, d), 7.55 (1H, s), 7.60 (1H, d), 7.92 (1H, s), 8.38 (1H, s), 12.87 (1H, s).

### Example B21

### 5,16,26-Trimethyl-7-oxa-4,5,13,16,17,20,22,27-octaazapentacyclo[22.3.1.0\hat{ }{2,6}.0\hat{ }{13,21}.0\hat{ }{14,19}]octacosa-1(27),2(6),3,14(19),17,20,24(28),25-octaene-15,23-dione

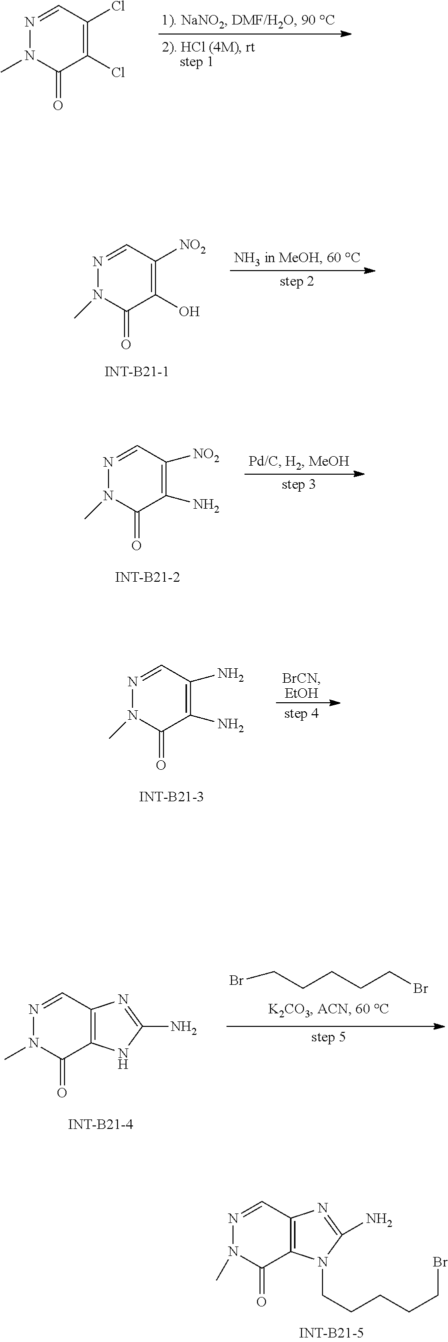

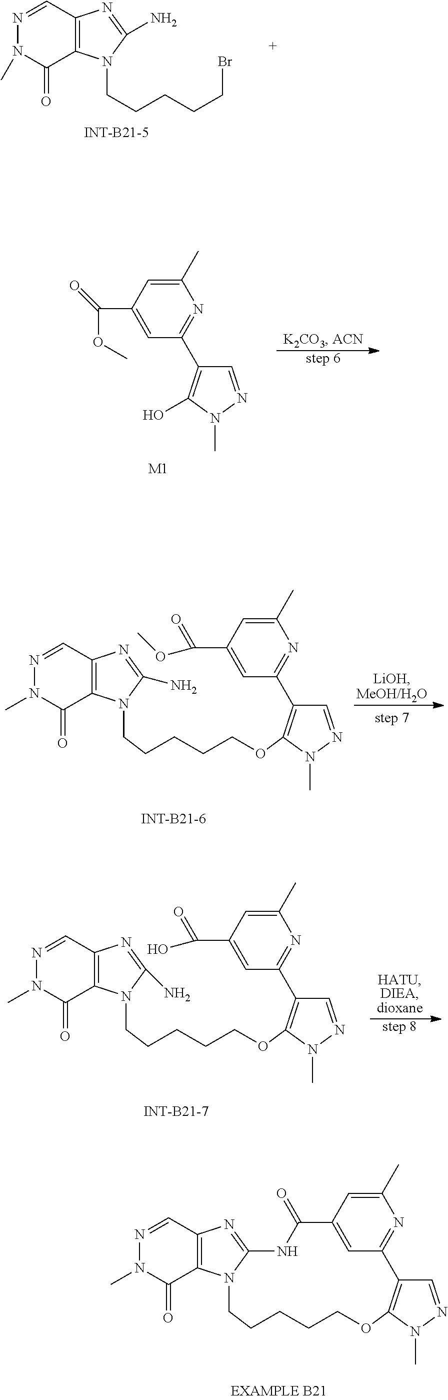

Step 1. Preparation of 4-hydroxy-2-methyl-5-nitropyridazin-3-one (INT-B21-1). A mixture of 4,5-dichloro-2-methylpyridazin-3-one (4.0 g, 22.34 mmol, 1 equiv) and NaNO₂ (6.1 g, 89.38 mmol, 4.0 equiv) in N,N-dimethylformamide (20 mL) was stirred for 24 h at 90 °C under nitrogen atmosphere. The mixture was cooled to room temperature and concentrated under reduced pressure. The residue was redissolved in warm 4M HCl (20 mL) and allowed to cool to room temperature. The solid was filtered and triturated with ether (100 mL) to give 1.0 g of 4-hydroxy-2-methyl-5-nitropyridazin-3-one (INT-B21-1) as a yellow solid (26%). ¹H NMR (DMSO-d₆, 400 MHz) δ 3.56 (3H, s), 8.20 (1H, s).

Step 2. Preparation of 4-amino-2-methyl-5-nitropyridazin-3-one (INT-B21-2). A mixture of 4-hydroxy-2-methyl-5-nitropyridazin-3-one (2.5 g, 14.61 mmol, 1 equiv) and NH₃ in methanol (60 mL) was stirred for 20 h at 60 °C under nitrogen atmosphere. The mixture was cooled to room temperature and concentrated under reduced pressure. The residue was purified by C18 FLASH using water and acetonitrile as mobile phase. Fractions containing the desired compound were evaporated to dryness to give to give 800 mg of 4-amino-2-methyl-5-nitropyridazin-3-one (INT-B21-2) as a yellow solid (32%). ¹H NMR (400 MHz, DMSO-d₆) δ 3.60 (s, 3H), 8.26 (s, 1H), 8.83 (s, 2H).

Step 3. Preparation of 4,5-diamino-2-methylpyridazin-3-one (INT-B21-3). A mixture of 4-amino-2-methyl-5-nitropyridazin-3-one (340 mg, 2.00 mmol, 1 equiv) and Pd/C (106 mg, 1.00 mmol, 0.5 equiv) in methanol (15 mL) was stirred at room temperature for 3 h under hydrogen atmosphere. The resulting mixture was filtered and the filter cake was washed with methanol (50 mL). The solution was concentrated under reduced pressure to give 200 mg of 4,5-diamino-2-methylpyridazin-3-one (INT-B21-3) as a light yellow solid (71%). This material was used for the next reaction without purification. LCMS: m/z (ESI), [M+H]⁺=141.00.

Step 4. Preparation of 2-amino-5-methyl-3H-imidazo[4,5-d]pyridazin-4-one (INT-B21-4). A mixture of 4,5-diamino-2-methylpyridazin-3-one (160 mg, 1.14 mmol, 1 equiv) and cyanogen bromide (181 mg, 1.71 mmol, 1.5 equiv) in ethanol (5 mL) was stirred for 16 h at room temperature under nitrogen atmosphere. The mixture was concentrated under reduced pressure and the residue was purified on Prep-TLC eluted with dichloromethane/methanol (10/1) to give 160 mg of 2-amino-5-methyl-3H-imidazo[4,5-d]pyridazin-4-one (INT-B21-4) as a yellow solid (59%). LCMS: m/z (ESI), [M+H]⁺=166.00.

Step 5. Preparation of 2-amino-3-(5-bromopentyl)-5-methylimidazo[4,5-d]pyridazin-4-one (INT-B21-5). A mixture of 2-amino-5-methyl-3H-imidazo[4,5-d]pyridazin-4-one (100 mg, 0.60 mmol, 1 equiv), 1,5-dibromopentane (409 mg, 1.78 mmol, 3.0 equiv), and K₂CO₃ (246.02 mg, 1.78 mmol, 3.0 equiv) in acetonitrile (10 mL) was stirred for 18 h at 60 °C under nitrogen atmosphere and cooled to room temperature. The reaction was quenched with water (30 mL) and the mixture was extracted with ethyl acetate (3×30 mL). The combined organic layers were washed with brine (3×30 mL), dried over anhydrous Na₂SO₄, and concentrated under reduced pressure. The residue was purified by C18 FLASH using water and acetonitrile as mobile phase. Fractions containing the desired compound were evaporated to dryness to give 70 mg of 2-amino-3-(5-bromopentyl)-5-methylimidazo[4,5-d]pyridazin-4-one (INT-B21-5) as a yellow solid (37%). LCMS: m/z (ESI), [M+H]⁺=315.95.

Step 6. Preparation of methyl 2-{5-[(5-{2-amino-6-methyl-7-oxoimidazo[4,5-d]pyridazin-1-yl}pentyl)oxy]-1-methylpyrazol-4-yl}-6-methylpyridine-4-carboxylate (INT-B21-6). A mixture of 2-amino-3-(5-bromopentyl)-5-methylimidazo[4,5-d]pyridazin-4-one (55 mg, 0.17 mmol, 1 equiv), methyl 2-(5-hydroxy-1-methylpyrazol-4-yl)-6-methylpyridine-4-carboxylate (M1, 65 mg, 0.26 mmol, 1.5 equiv) and K₂CO₃ (72 mg, 0.52 mmol, 3.0 equiv) in acetonitrile (2.0 mL) was stirred for 3 h at 60 °C under nitrogen atmosphere and then cooled to room temperature. The reaction was quenched with water (30 mL) and the mixture was extracted with ethyl acetate (3×30 mL). The combined organic layers were washed with brine (3×30 mL), dried over anhydrous Na₂SO₄, and concentrated under reduced pressure. The residue was purified by C18 FLASH using water and acetonitrile as mobile phase. Fractions containing the desired compound were evaporated to dryness to give 25 mg of methyl 2-{5-[(5-{2-amino-6-methyl-7-oxoimidazo[4,5-d]pyridazin-1-yl}pentyl)oxy]-1-methylpyrazol-4-yl}-6-methylpyridine-4-carboxylate (INT-B21-6) as a light yellow solid (29%). LCMS: m/z (ESI), [M+H]⁺=481.20.

Step 7. Preparation of 2-{5-[(5-{2-amino-6-methyl-7-oxoimidazo[4,5-d]pyridazin-1-yl}pentyl)oxy]-1-methylpyrazol-4-yl}-6-methylpyridine-4-carboxylic acid (INT-B21-7).

A mixture of methyl 2-{5-[(5-{2-amino-6-methyl-7-oxoimidazo[4,5-d]pyridazin-1-yl}pentyl)oxy]-1-methylpyrazol-4-yl}-6-methylpyridine-4-carboxylate (25 mg, 0.05 mmol, 1 equiv) and LiOH·H₂O (4.4 mg, 0.10 mmol, 2.0 equiv) in methanol (0.5 mL) and H₂O (1.0 mL) was stirred for 2 h at 20 °C under nitrogen atmosphere. The mixture was concentrated under reduced pressure. The residue was purified by C18 FLASH using water and acetonitrile as mobile phase. Fractions containing the desired compound were evaporated to dryness to give 15 mg of 2-{5-[(5-{2-amino-6-methyl-7-oxoimidazo[4,5-d]pyridazin-1-yl}pentyl)oxy]-1-methylpyrazol-4-yl}-6-methylpyridine-4-carboxylic acid (INT-B21-7) as a light yellow solid (61%). LCMS: m/z (ESI), [M+H]⁺=467.15.

Step 8. Preparation of 5,16,26-trimethyl-7-oxa-4,5,13,16,17,20,22,27-octaazapentacyclo[22.3.1.0\hat{ }{2,6}.0\hat{ }{13,21}.0\hat{ }{14,19}]octacosa-1(27),2(6),3,14(19),17,20,24(28),25-octaene-15,23-dione (EXAMPLE B21). A mixture of 2-{5-[(5-{2-amino-6-methyl-7-oxoimidazo[4,5-d]pyridazin-1-yl}pentyl)oxy]-1-methylpyrazol-4-yl}-6-methylpyridine-4-carboxylic acid (15 mg, 0.03 mmol, 1 equiv), N,N,N,N-tetramethyl-O-(7-azabenzotriazol-1-yl)uronium hexafluorophospate (24 mg, 0.06 mmol, 2.0 equiv) and N,N-diisopropylethylamine (12 mg, 0.10 mmol, 3.0 equiv) in 1,4-dioxane (1.0 mL) was stirred at room temperature for 18 h under nitrogen atmosphere. The mixture was concentrated under reduced pressure. The residue was treated with dimethyl sulfoxide (2 mL). The solid was collected by filtration and washed with methanol (3×5 mL) to give 14 mg of 5,16,26-trimethyl-7-oxa-4,5,13,16,17,20,22,27-octaazapentacyclo[22.3.1.0\hat{ }{2,6}.0\hat{ }{13,21}.0\hat{ }{14,19}]octacosa-1(27),2(6),3,14(19),17,20,24(28),25-octaene-15,23-dione (EXAMPLE B21) as a white solid (99%). LCMS: m/z (ESI), [M+H]⁺=449.15. ¹H NMR (CDCl₃, 400 MHz) δ 1.72-1.82 (2H, m), 2.00-2.14 (4H, m), 2.56 (3H, s), 3.73 (3H, s), 3.76 (3H, s), 4.20 (2H, t), 4.47 (2H, t), 7.56 (1H, s), 7.92 (1H, s), 8.22 (1H, s), 8.37 (1H, s).

### Example B22

### 16-(2-Hydroxyethyl)-5,26-dimethyl-7-oxa-4,5,13,16,20,22,27-heptaazapentacyclo[22.3.1.0\hat{ }{2,6}.0\hat{ }{13,21}.0\hat{ }{14,19}]octacosa-1(27),2(6),3,14(19),17,20,24(28),25-octaene-15,23-dione

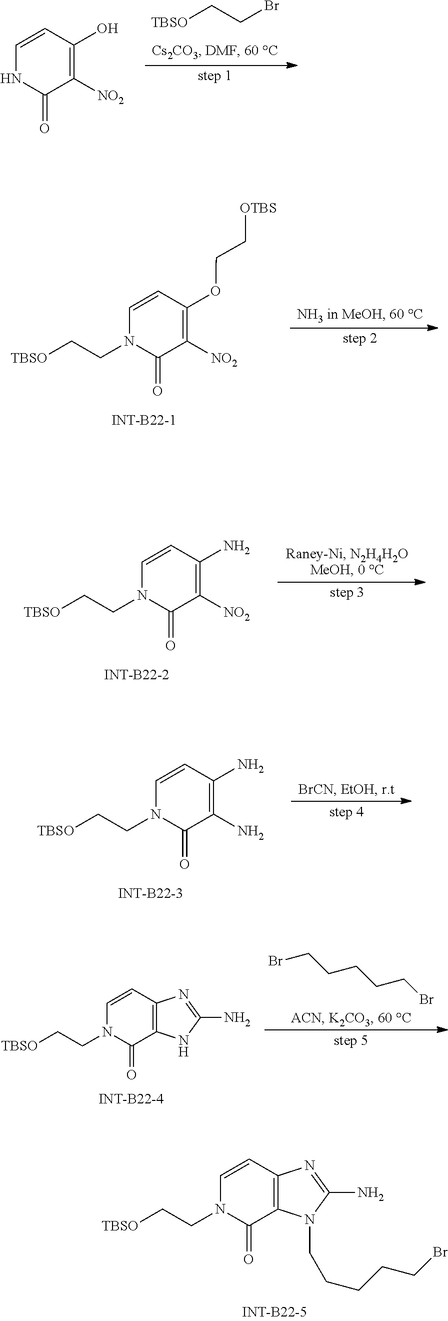

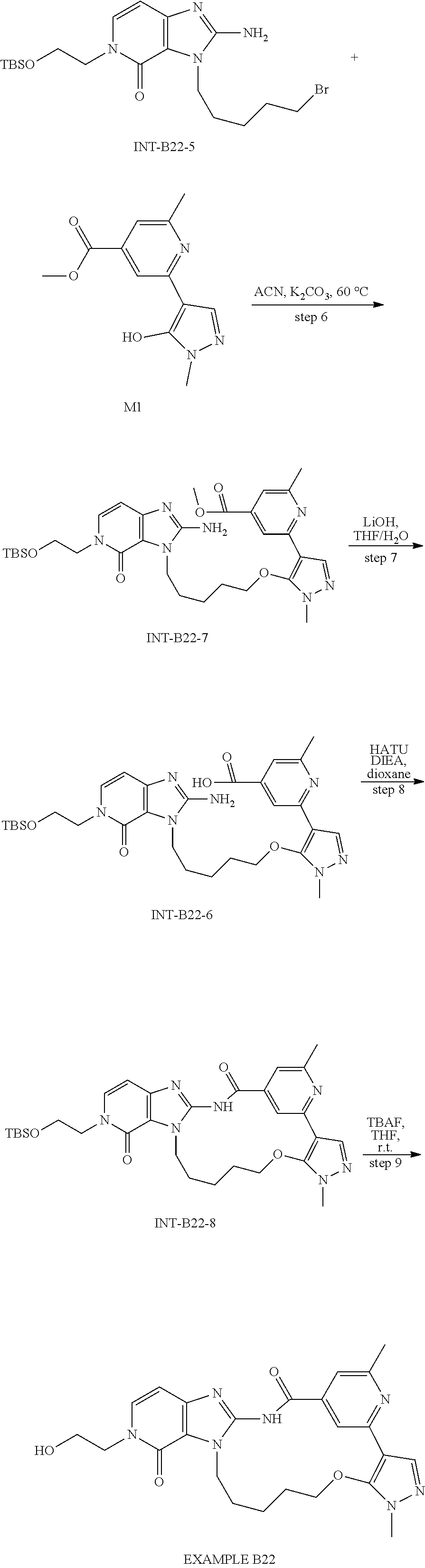

Step 1. Preparation of 4-(2-{[(tert-butyldimethylsilyl)oxy]amino}ethoxy)-1-(2-{[(tert-butyldimethylsilyl)oxy]amino}ethyl)-3-nitropyridin-2-one (INT-B22-1). A mixture of 4-hydroxy-3-nitro-1H-pyridin-2-one (7 g, 44.84 mmol, 1 equiv), cesium carbonate (43.8 g, 134.53 mmol, 3 equiv), and (2-bromoethoxy)(tert-butyl)dimethylsilane (42.9 g, 179.38 mmol, 4 equiv) in N,N-dimethylformamide (100 mL) was stirred for 12 h at 60 °C. After being cooled to room temperature, the reaction was treated with water (600 mL) and the mixture was extracted with ethyl acetate (3×500 mL). The combined organic layers were washed with brine (3×500 mL), dried over anhydrous Na₂SO₄, and concentrated under reduced pressure. The residue was purified by silica gel column chromatography eluted with petroleum ether/ethyl acetate (2/1) to give 8.6 g of 4-(2-{[(tert-butyldimethylsilyl)oxy]amino}ethoxy)-1-(2-{[(tert-butyldimethylsilyl)oxy]amino}ethyl)-3-nitropyridin-2-one (INT-B22-1) as a yellow solid (37%). LCMS: m/z (ESI), [M+H]⁺=473.30. ¹H NMR (400 MHz, DMSO-d₆) δ 0-0.19 (12H, m), 0.69-0.97 (18H, m), 3.75-3.96 (4H, m), 3.98-4.15 (2H, m), 4.20-4.46 (2H, m), 6.47-6.61 (1H, m), 7.81-7.96 (1H, m).

Step 2. Preparation of 4-amino-1-{2-[(tert-butyldimethylsilyl)oxy]ethyl}-3-nitropyridin-2-one (INT-B22-2). A mixture of 4-{2-[(tert-butyldimethylsilyl)oxy]ethoxy}-1-{2-[(tert-butyldimethylsilyl)oxy]ethyl}-3-nitropyridin-2-one (8.6 g, 18.01 mmol, 1 equiv) and NH₃ in methanol (60 mL) was stirred for 12 h at 60 °C. After being cooled to room temperature, the mixture was concentrated under reduced pressure to give 5.6 g of 4-amino-1-{2-[(tert-butyldimethylsilyl)oxy]ethyl}-3-nitropyridin-2-one (INT-B22-2) as a yellow solid (96%). This material was used for the next reaction without further purification. LCMS: m/z (ESI), [M+H]⁺=314.10. ¹H NMR (400 MHz, DMSO-d₆) δ 0.05 (6H, s), 0.82 (9H, s), 3.72-3.78 (2H, m), 3.82-3.88 (2H, m), 5.84-5.89 (1H, m), 7.45 (1H, d), 8.04 (2H, s).

Step 3. Preparation of 3,4-diamino-1-{2-[(tert-butyldimethylsilyl)oxy]ethyl}pyridin-2-one (INT-B22-3). To a stirred mixture of 4-amino-1-{2-[(tert-butyldimethylsilyl)oxy]ethyl}-3-nitropyridin-2-one (4.4 g, 13.89 mmol, 1 equiv) and Raney-Ni (1.10 g, 12.79 mmol, 0.92 equiv) in methanol (200 mL) were added hydrazine hydrate (4.26 g, 83.39 mmol, 6 equiv) at 0 °C under nitrogen atmosphere. The resulting mixture was stirred for 4 h at room temperature and filtered. The filter cake was washed with methanol (3×100 mL). The solution was concentrated under reduced pressure. The residue was purified by silica gel column chromatography eluted with petroleum dichloromethane/methanol (10/1) to give 3.7 g of 3,4-diamino-1-{2-[(tert-butyldimethylsilyl)oxy]ethyl}pyridin-2-one (INT-B22-3) as a grey powder (93%). LCMS: m/z (EST), [M+H]⁺=284.15. ¹H NMR (400 MHz, DMSO-d₆) δ 0.07 (6H, s), 0.82 (9H, s), 3.74 (2H, t), 3.80-3.89 (4H, m), 5.11 (2H, s), 5.73 (1H, d), 6.79 (1H, d).

Step 4. Preparation of 2-amino-5-{2-[(tert-butyldimethylsilyl)oxy]ethyl}-3H-imidazo[4,5-c]pyridin-4-one (INT-B22-4), To a stirred mixture of 3,4-diamino-1-{2-[(tert-butyldimethylsilyl)oxy]ethyl}pyridin-2-one (3.7 g, 13.05 mmol, 1 equiv) in ethanol (60 mL) was added cyanogen bromide (2.1 g, 19.58 mmol, 1.5 equiv) at room temperature under nitrogen atmosphere. The resulting mixture was stirred for 4 h at room temperature and concentrated under reduced pressure. The residue was purified by silica gel column chromatography and eluted with dichloromethane/methanol (7/1) to give 6.6 g of 2-amino-5-{2-[(tert-butyldimethylsilyl)oxy]ethyl}-3H-imidazo[4,5-c]pyridin-4-one (INT-B22-4) as a brown solid (95%). LCMS: m/z (ESI), [M+H]⁺=309.25. ¹H NMR (400 MHz, DMSO-d₆) δ 0.14 (6H, s), 0.71 (9H, s), 3.95 (2H, t), 4.36 (2H, t), 6.73 (1H, d), 7.88 (1H, d), 8.03 (2H, s).

Step 5. Preparation of 2-amino-3-(5-bromopentyl)-5-{2-[(tert-butyldimethylsilyl)oxy]ethyl}imidazo[4,5-c]pyridin-4-one (INT-B22-5). To a stirred mixture of 2-amino-5-{2-[(tert-butyldimethylsilyl)oxy]ethyl}-3H-imidazo[4,5-c]pyridin-4-one (6.6 g, 21.40 mmol, 1 equiv) and K₂CO₃ (5.9 g, 42.79 mmol, 2 equiv) in acetonitrile (100 mL) was added 1,5-dibromo-pentane (24.6 g, 106.99 mmol, 5 equiv) at room temperature. The resulting mixture was stirred for 4 h at 60 °C. After being cooled to room temperature, the mixture was filtered and the filtrate was concentrated under reduced pressure. The residue was purified by silica gel column chromatography and eluted with dichloromethane/methanol (10/1) to give 2.2 g of 2-amino-3-(5-bromopentyl)-5-{2-[(tert-butyldimethylsilyl)oxy]ethyl}imidazo[4,5-c]pyridin-4-one (INT-B22-5) as a brown solid (21%). LCMS: m/z (ESI), [M+H]⁺=457.20. ¹H NMR (400 MHz, DMSO-d₆) δ 0.12 (6H, s), 0.80 (9H, s), 1.31-1.43 (2H, m), 1.62-1.68 (2H, m), 1.78-1.84 (2H, m), 3.51 (2H, t), 3.80 (2H, t), 3.99 (2H, t), 4.19 (2H, t), 6.22 (1H, d), 6.50 (2H, s), 7.16 (1H, d).

Step 6. Preparation of methyl 2-(5-{[5-(2-amino-5-{2-[(tert-butyldimethylsilyl)oxy]ethyl}-4-oxoimidazo[4,5-c]pyridin-3-yl)pentyl]oxy}-1-methylpyrazol-4-yl)-6-methylpyridine-4-carboxylate (INT-B22-6). To a stirred mixture of 2-amino-3-(5-bromopentyl)-5-{2-[(tert-butyldimethylsilyl)oxy]ethyl}imidazo[4,5-c]pyridin-4-one (2.2 g, 4.81 mmol, 1 equiv) and K₂CO₃ (1.3 g, 9.62 mmol, 2 equiv) in acetonitrile (20 mL) was added methyl 2-(5-hydroxy-1-methylpyrazol-4-yl)-6-methylpyridine-4-carboxylate (1.19 g, 4.81 mmol, 1 equiv) in acetonitrile (5 mL) at room temperature under nitrogen atmosphere. The resulting mixture was stirred for 12 h at 60 °C. After being cooled to room temperature, the reaction was quenched with water (100 mL) and the mixture was extracted with ethyl acetate (3×100 mL). The combined organic layers were washed with brine (3×100 mL), dried over anhydrous Na₂SO₄, and concentrated under reduced pressure. The residue was purified on a silica gel column eluted with dichloromethane/methanol (10/1) to give 2.5 g of methyl 2-(5-{[5-(2-amino-5-{2-[(tert-butyldimethylsilyl)oxy]ethyl}-4-oxoimidazo[4,5-c]pyridin-3-yl)pentyl]oxy}-1-methylpyrazol-4-yl)-6-methylpyridine-4-carboxylate (INT-B22-6) as a brown solid (80%). LCMS: m/z (ESI), [M+H]⁺=610.30.

Step 7. Preparation of 2-(5-{[5-(2-amino-5-{2-[(tert-butyldimethylsilyl)oxy]ethyl}-4-oxoimidazo[4,5-c]pyridin-3-yl)pentyl]oxy}-1-methylpyrazol-4-yl)-6-methylpyridine-4-carboxylic acid (INT-B22-7). To a stirred mixture of methyl 2-(5-{[5-(2-amino-5-{2-[(tert-butyldimethylsilyl)oxy]ethyl}-4-oxoimidazo[4,5-c]pyridin-3-yl)pentyl]oxy}-1-methylpyrazol-4-yl)-6-methylpyridine-4-carboxylate (2.5 g, 4.00 mmol, 1 equiv) in tetrahydrofuran (20 mL) and H₂O (5 mL) was added lithium hydroxide (336 mg, 8.01 mmol, 2 equiv) at room temperature under nitrogen atmosphere. The reaction mixture was stirred for 3 h. The resulting mixture was concentrated under reduced pressure and purified by reverse flash chromatography eluted with 10% to 50% acetonitrile in water to give 2.0 g of 2-(5-{[5-(2-amino-5-{2-[(tert-butyldimethylsilyl)oxy]ethyl}-4-oxoimidazo[4,5-c]pyridin-3-yl)pentyl]oxy}-1-methylpyrazol-4-yl)-6-methylpyridine-4-carboxylic acid (INT-B22-7) as a brown solid (75%). LCMS: m/z (ESI), [M+H]⁺=610.30.

Step 8. Preparation of 16-{2-[(tert-butyldimethylsilyl)oxy]ethyl}-5,26-dimethyl-7-oxa-4,5,13,16,20,22,27-heptaazapentacyclo[22.3.1.0\hat{ }{2,6}.0\hat{ }{13,21}.0\hat{ }{14,19}]octacosa-1(27),2(6),3,14(19),17,20,24(28),25-octaene-15,23-dione (INT-B22-8). To a stirred mixture of 2-(5-{[5-(2-amino-5-{2-[(tert-butyldimethylsilyl)oxy]ethyl}-4-oxoimidazo[4,5-c]pyridin-3-yl)pentyl]oxy}-1-methylpyrazol-4-yl)-6-methylpyridine-4-carboxylic acid (20 mg, 0.03 mmol, 1 equiv) and N,N,N,N-tetramethyl-O-(7-azabenzotriazol-1-yl)uronium hexafluorophospate (18.71 mg, 0.05 mmol, 1.5 equiv) in dioxane (4 mL) was added N,N-diisopropylethylamine (12.72 mg, 0.10 mmol, 3 equiv) at room temperature under nitrogen atmosphere. After being stirred for 12 h at room temperature, the reaction was quenched with water (100 mL) and the mixture was extracted with dichloromethane (2×80 mL). The combined organic layers were washed with brine (3×100 mL), dried over anhydrous Na₂SO₄, and concentrated under reduced pressure. The residue was purified by prep-TLC eluted with dichloromethane/methanol (10/1) to give 8 mg of 16-{2-[(tert-butyldimethylsilyl)oxy]ethyl}-5,26-dimethyl-7-oxa-4,5,13,16,20,22,27-heptaazapentacyclo[22.3.1.0\hat{ }{2,6}.0\hat{ }{13,21}.0\hat{ }{14,19}]octacosa-1(27),2(6),3,14(19),17,20,24(28),25-octaene-15,23-dione (INT-B22-8) as a yellow solid (39%). LCMS: m/z (EST), [M+H]⁺=592.35. ¹H NMR (400 MHz, CD₃OD) δ 0.09 (6H, s), 0.83 (9H, s), 1.51-1.63 (2H, m), 1.76-1.91 (4H, m), 2.56 (3H, d), 3.72 (3H, s), 3.89-3.96 (2H, m), 4.09-4.16 (4H, m), 4.25-4.35 (2H, m), 6.39 (1H, d), 7.25 (1H, d), 7.51 (1H, d), 7.84 (1H, s), 7.88-7.93 (1H, m).

Step 9. 16-(2-hydroxyethyl)-5,26-dimethyl-7-oxa-4,5,13,16,20,22,27-heptaazapentacyclo[22.3.1.0\hat{ }{2,6}.0\hat{ }{13,21}.0\hat{ }{14,19}]octacosa-1(27),2(6),3,14(19),17,20,24(28),25-octaene-15,23-dione (EXAMPLE B22). A mixture of 16-{2-[(tert-butyldimethylsilyl)oxy]ethyl}-5,26-dimethyl-7-oxa-4,5,13,16,20,22,27-heptaazapentacyclo[22.3.1.0\hat{ }{2,6}.0\hat{ }{13,21}.0\hat{ }{14,19}]octacosa-1(27),2(6),3,14(19),17,20,24(28),25-octaene-15,23-dione (40 mg, 0.068 mmol, 1 equiv) and tetrabutylammonium fluoride (35.35 mg, 0.136 mmol, 2 equiv) in tetrahydrofuran (2 mL) was stirred for 4 h at room temperature. The resulting mixture was concentrated under reduced pressure. The residue was purified by reverse flash chromatography eluted with 10% to 100% MeOH in water to give 7.7 mg of 16-(2-hydroxyethyl)-5,26-dimethyl-7-oxa-4,5,13,16,20,22,27-heptaazapentacyclo[22.3.1.0\hat{ }{2,6}.0\hat{ }{13,21}.0\hat{ }{14,19}]octacosa-1(27),2(6),3,14(19),17,20,24(28),25-octaene-15,23-dione (EXAMPLE B22) as a yellow solid (21%). LCMS: m/z (ESI), [M+H]⁺=478.15. ¹H NMR (CD₃OD, 400 MHz) δ 1.85-1.93 (2H, m), 2.11-2.23 (4H, m), 2.89 (3H, s), 3.87 (3H, s), 3.88-3.94 (2H, m), 4.22 (2H, t), 4.41 (2H, t), 4.60-4.69 (2H, m), 6.69 (1H, d), 7.59 (1H, d), 8.10 (1H, s), 8.36 (1H, s), 8.95 (1H, s).

### Example B23

### 18-[2-(Dimethylamino)ethyl]-5,30-dimethyl-7-oxa-4,5,13,18,24,26,31-heptaazahexacyclo[26.3.1.0\hat{ }{2,6}.0\hat{ }{13,25}.0\hat{ }{14,23}.0\hat{ }{15,20}]dotriaconta-1(31),2(6),3,14,20,22,24,28(32),29-nonaen-27-one

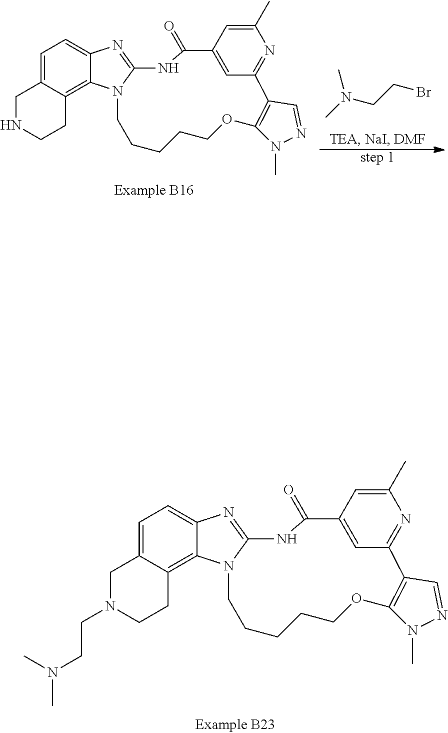

Step 1. Preparation of 18-[2-(dimethylamino)ethyl]-5,30-dimethyl-7-oxa-4,5,13,18,24,26,31-heptaazahexacyclo[26.3.1.0\hat{ }{2,6}.0\hat{ }{13,25}.0\hat{ }{14,23}.0\hat{ }{15,20}]dotriaconta-1(31),2(6),3,14,20,22,24,28(32),29-nonaen-27-one (EXAMPLE B23). A mixture of 5,30-dimethyl-7-oxa-4,5,13,18,24,26,31-heptaazahexacyclo[26.3.1.0\hat{ }{2,6}.0\hat{ }{13,25}.0\hat{ }{14,23}.0\hat{ }{15,20}]dotriaconta-1(31),2(6),3,14,20,22,24,28(32),29-nonaen-27-one (EXAMPLE B16, 50 mg, 0.10 mmol, 1 equiv), (2-bromoethyl)dimethylamine (32 mg, 0.21 mmol, 2 equiv), trimethylamine (21 mg, 0.21 mmol, 2 equiv), and NaI (3.1 mg, 0.02 mmol, 0.2 equiv) in N,N-dimethylformamide (2 mL) was stirred for 6 h at 60 °C. The mixture was cooled to room temperature. The reaction was quenched with water (20 mL) and the mixture was extracted with ethyl acetate (3×20 mL). The combined organic layers were washed with brine (3×20 mL), dried over anhydrous Na₂SO₄, and concentrated under reduced pressure. The residue was purified by prep-HPLC with XBridge Prep OBD C18 column using water containing 10 mmol/L NH₄HCO₃ and 0.1% NH₃—H₂O and acetonitrile as mobile phase. Fractions containing the desired compound were evaporated to dryness to give 5.4 mg of 18-[2-(dimethylamino)ethyl]-5,30-dimethyl-7-oxa-4,5,13,18,24,26,31-heptaazahexacyclo[26.3.1.0\hat{ }{2,6}.0\hat{ }{13,25}.0\hat{ }{14,23}.0\hat{ }{15,20}1]dotriaconta-1(31),2(6),3,14,20,22,24,28(32),29-nonaen-27-one (EXAMPLE B23) as a white solid (8%). LCMS: m/z (EST), [M+H]⁺=543.40. ¹H NMR (DMSO-d₆, 400 MHz) δ 1.79-1.83 (2H, m), 2.02-2.06 (4H, m), 2.21 (6H, s), 2.56 (3H, s), 2.54-2.64 (2H, m), 2.77-2.81 (2H, m), 3.15-3.44 (4H, m), 3.67 (2H, s), 3.73 (3H, s), 4.22 (2H, t), 4.35 (2H, t), 6.94 (1H, d), 7.35 (1H, d), 7.57 (1H, s), 7.93 (1H, s), 8.41 (1H, s), 12.66 (1H, s).

### Example B27

### 5-[4-(dimethylamino)piperidin-1-yl]-15,21-dimethyl-23-oxa-2,9,11,16,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,15,17(34),18(22),19-nonaen-12-one

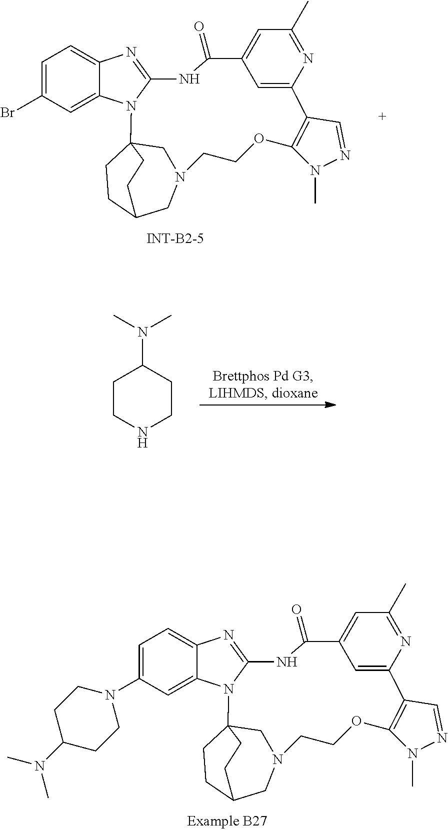

To a stirred mixture of 5-bromo-15,21-dimethyl-23-oxa-2,9,11,16,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,15,17(34),18(22),19-nonaen-12-one (INT-B2-5, 70 mg, 0.12 mmol, 1 equiv), N,N-dimethylpiperidin-4-amine (93 mg, 0.73 mmol, 6 equiv) and BrettPhos Pd G₃ (33 mg, 0.04 mmol, 0.3 equiv) in 1,4-dioxane (6 mL) was added LiHMDS (1.5 mL, 1.45 mmol, 12 equiv) dropwise at room temperature under nitrogen atmosphere. The mixture was stirred for 2 h at 60 °C under nitrogen atmosphere and cooled to room temperature. The reaction was quenched with saturated NH₄Cl (aq.) (20 mL) at room temperature and the mixture was extracted with CH₂Cl₂ (3×50 mL). The combined organic layers were dried over anhydrous Na₂SO₄ and concentrated under reduced pressure. The residue was purified by prep-TLC, eluted with dichloromethane/methanol (10:1). The product was further purified by prep-HPLC with XBridge Prep OBD C18 column eluting with water containing 0.1% NH₄HCO₃ and acetonitrile. Fractions containing the desired compound were evaporated to dryness to give 20.4 mg of 5-[4-(dimethylamino)piperidin-1-yl]-15,21-dimethyl-23-oxa-2,9,11,16,20,21,26-heptaazaheptacyclo[26.2.2.1\hat{ }{1,26}.1\hat{ }{13,17}.0\hat{ }{2,10}.0\hat{ }{3,8}.0\hat{ }{18,22}]tetratriaconta-3,5,7,9,13,15,17(34),18(22),19-nonaen-12-one (EXAMPLE B27) as a yellow solid (26%). LCMS: m/z (EST), [M+H]⁺=624.30 ¹H NMR (DMSO-d₆, 400 MHz) δ 1.47-1.60 (2H, m), 1.88-2.04 (6H, m), 2.06-2.15 (3H, m), 2.21-2.28 (7H, m), 2.54 (3H, s), 2.61-2.71 (2H, m), 2.81 (2H, d), 2.86 (2H, d), 3.46 (4H, d), 3.56 (2H, d), 3.76 (3H, s), 4.38 (2H, t), 6.94 (1H, dd), 7.36 (1H, d), 7.48 (1H, d), 7.61 (1H, d), 7.89 (1H, s), 8.76 (1H, s), 13.20 (1H, s).

EXAMPLE B28-EXAMPLE B39 (Table 3) were prepared using the same reaction conditions described above for the synthesis of EXAMPLE B27 from INT-B2-5.

TABLE 3

| Example | | [M + H]⁺ | |
|---|---|---|---|
| No. | Structure | (observed) | ¹H NMR δ (ppm) |
| B28 | 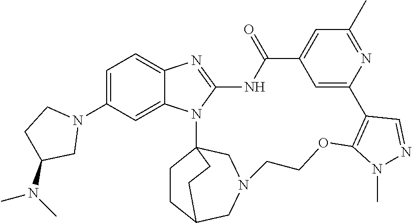 [SMILES: Cc1cc2cc(n1)-c1cnn(C)c1OCCN1CC3CCC(CC3)(C1)n1c(nc3ccc(N4CC[C@H](N(C)C)C4)cc31)NC2=O] | 610.25 | ¹H NMR (DMSO-d₆, 400 MHz) δ 1.78- 2.01 (5H, m), 2.04-2.16 (4H, m), 2.22 (6H, s), 2.54 (3H, s), 2.78-2.96 (5H, m), 3.07 (1H, t), 3.21-3.27 (1H, m), 3.33- 3.38 (1H, m), 3.40-3.65 (5H, m), 3.76 (3H, s), 4.37 (2H, s), 6.54 (1H, d), 6.90 (1H, s), 7.45 (1H, d), 7.61 (1H, s), 7.89 (1H, s), 8.76 (1H, s), 13.15 (1H, s) |
| B29 | 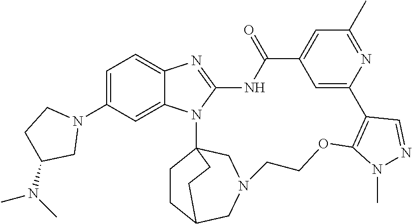 [SMILES: Cc1cc2cc(n1)-c1cnn(C)c1OCCN1CC3CCC(CC3)(C1)n1c(nc3ccc(N4CC[C@@H](N(C)C)C4)cc31)NC2=O] | 610.35 | ¹H NMR (DMSO-d₆, 400 MHz) δ 1.77- 2.02 (6H, m), 2.01-2.20 (4H, m), 2.22 (6H, s), 2.54 (3H, s), 2.77-2.90 (5H, m), 3.06 (1H, t), 3.26 (1H, d), 3.40-3.55 (5H, m), 3.76 (3H, s), 4.37 (2H, t), 6.48-6.71 (1H, m), 6.90 (1H, s), 7.45 (1H, d), 7.61 (1H, s), 7.89 (1H, s), 8.76 (IH, s), 13.15 (1H, s). |
| B30 | 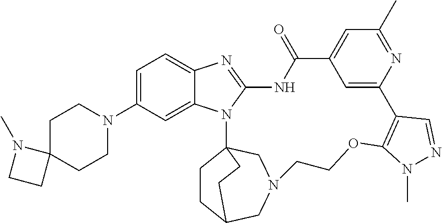 [SMILES: Cc1cc2cc(n1)-c1cnn(C)c1OCCN1CC3CCC(CC3)(C1)n1c(nc3ccc(N4CCC5(CC4)CCN5C)cc31)NC2=O] | 636.20 | ¹H NMR (DMSO-d₆, 400 MHz) δ 1.69- 1.78 (2H, m), 1.79-1.84 (2H, m), 1.85- 2.02 (7H, m), 2.01-2.11 (3H, m), 2.15 (3H, s), 2.54 (3H, s), 2.60-2.72 (2H, m), 2.75-2.90 (4H, m), 3.09-3.18 (2H, m), 3.43-3.51 (5H, m), 3.76 (3H, s), 4.38 (2H, d), 6.92-6.98 (1H, m), 7.35 (1H, s), 7.48 (1H, d), 7.61 (1H, s), 7.89 (1H, s), 8.76 (1H, s). |
| B31 | 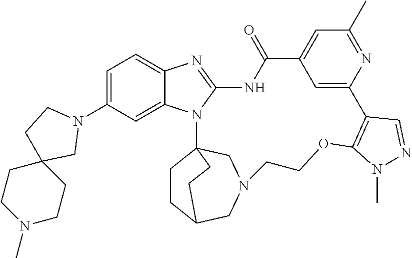 [SMILES: Cc1cc2cc(n1)-c1cnn(C)c1OCCN1CC3CCC(CC3)(C1)n1c(nc3ccc(N4CCC5(CCN(C)CC5)C4)cc31)NC2=O] | 650.20 | ¹H NMR (DMSO-d₆, 400 MHz) δ 1.50- 1.72 (4H, m), 1.80-2.02 (7H, m), 2.01- 2.08 (3H, m), 2.27 (3H, s), 2.32-2.50 (5H, m), 2.54 (3H, s), 2.81 (2H, s), 2.86 (2H, d), 3.10 (2H, s), 3.40-3.52 (4H, m), 3.76 (3H, s), 4.37 (2H, t), 6.51 (1H, d), 6.89 (1H, s), 7.44 (1H, d), 7.61 (1H, s), 7.89 (1H, s), 8.76 (1H, s), 13.15 (1H, s) |
| B32 | 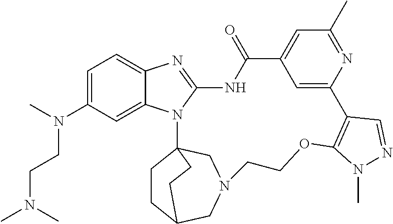 [SMILES: Cc1cc2cc(n1)-c1cnn(C)c1OCCN1CC3CCC(CC3)(C1)n1c(nc3ccc(N(C)CCN(C)C)cc31)NC2=O] | 598.25 | ¹H NMR (DMSO-d₆, 400 MHz) δ 1.85- 2.02 (4H, m), 2.04-2.18 (3H, m), 2.25 (6H, s), 2.41-2.48 (2H, m), 2.54 (3H, s), 2.81 (2H, d), 2.87 (2H, d), 2.91 (3H, s), 3.45-3.53 (6H, m), 3.76 (3H, s), 4.37 (2H, t), 6.72 (1H, dd), 7.13 (1H, d), 7.45 (1H, d), 7.61 (1H, d), 7.89 (1H, s), 8.76 (1H, s), 13.15 (1H, s). |
| B33 | 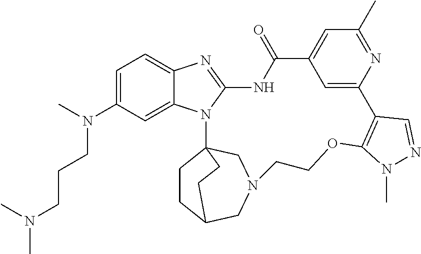 [SMILES: Cc1cc2cc(n1)-c1cnn(C)c1OCCN1CC3CCC(CC3)(C1)n1c(nc3ccc(N(C)CCCN(C)C)cc31)NC2=O] | 612.25 | ¹H NMR (DMSO-d₆, 400 MHz) δ 1.61- 1.69 (2H, m), 1.82-2.01 (4H, m), 2.01- 2.11 (3H, m), 2.16 (6H, s), 2.25 (2H, t), 2.54 (3H, s), 2.81 (2H, s), 2.87 (5H, s), 3.31-3.32 (1H, m), 3.42-3.53 (5H, m), 3.76 (3H, s), 4.38 (2H, s), 6.73 (1H, d), 7.15 (1H, s), 7.44 (1H, d), 7.61 (1H, s), 7.89 (1H, s), 8.76 (1H, s), 13.15 (1H, s). |
| B34 | 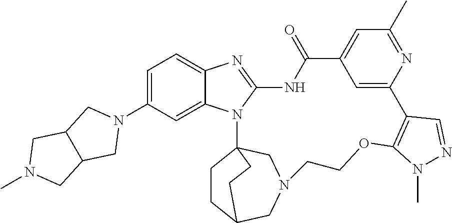 [SMILES: Cc1cc2cc(n1)-c1cnn(C)c1OCCN1CC3CCC(CC3)(C1)n1c(nc3ccc(N4CC5CN(C)CC5C4)cc31)NC2=O] | 622.25 | ¹H NMR (DMSO-d₆, 400 MHz) δ 1.85- 1.98 (4H, m), 2.03-3.15 (3H, m), 2.25 (3H, s), 2.42-2.48 (2H, m), 2.54 (3H, s), 2.56-2.68 (2H, m), 2.81 (2H, d), 2.87 (4H, d), 3.08-3.13 (2H, m), 3.34-3.39 (2H, m), 3.46 (4H, d), 3.76 (3H, s), 4.38 (2H, s), 6.62-6.69 (1H, m), 7.04 (1H, s), 7.46 (1H, d), 7.61 (1H, s), 7.89 (1H, s), 8.76 (1H, s), 13.17 (1H, s). |
| B35 | 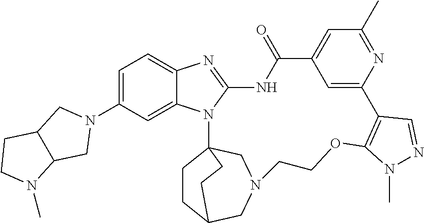 [SMILES: Cc1cc2cc(n1)-c1cnn(C)c1OCCN1CC3CCC(CC3)(C1)n1c(nc3ccc(N4CC5CCN(C)C5C4)cc31)NC2=O] | 622.20 | ¹H NMR (DMSO-d₆, 400 MHz) δ 1.57- 1.70 (1H, m), 1.84-2.01 (4H, m), 2.02- 2.13 (4H, m), 2.15-2.37 (4H, m), 2.54 (3H, s), 2.81 (2H, d), 2.83-2.96 (4H, d), 2.99 (1H, t), 3.14 (2H, dd), 3.25- 3.42 (2H, m), 3.46-3.52 (4H, m), 3.76 (3H, s), 4.37 (2H, t), 6.54-6.68 (1H, m), 6.97 (1H, s), 7.45 (1H, d), 7.61 (1H, s), 7.89 (1H, s), 8.76 (1H, s), 13.16 (1H, s) |
| B36 | 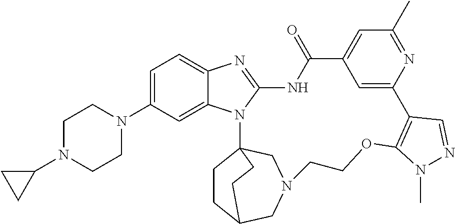 [SMILES: Cc1cc2cc(n1)-c1cnn(C)c1OCCN1CC3CCC(CC3)(C1)n1c(nc3ccc(N4CCN(C5CC5)CC4)cc31)NC2=O] | 622.25 | ¹H NMR (DMSO-d₆, 400 MHz) δ 0.34 (2H, s), 0.45 (2H, s), 1.61-1.71 (1H, m), 1.85-2.11 (7H, m), 2.53-2.57 (3H, m), 2.66-2.75 (4H, m), 2.84 (4H, d), 2.99- 3.11 (4H, m), 3.46 (4H, d), 3.76 (3H, s), 4.38 (2H, s), 6.94 (1H, d), 7.34 (1H, s), 7.49 (1H, d), 7.61 (1H, s), 7.89 (1H, s), 8.76 (1H, s), 13.21 (1H, s). |
| B37 | 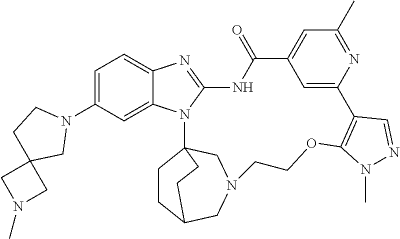 [SMILES: Cc1cc2cc(n1)-c1cnn(C)c1OCCN1CC3CCC(CC3)(C1)n1c(nc3ccc(N4CCC5(CN(C)C5)C4)cc31)NC2=O] | 622.30 | ¹H NMR (DMSO-d₆, 400 MHz) δ 1.84- 2.02 (4H, m), 2.03-2.15 (5H, m), 2.24 (3H, s), 2.54 (3H, s), 2.79-2.89 (4H, m), 3.13 (4H, s), 3.24 (2H, t),3.37 (2H, s), 3.41-3.53 (4H, m), 3.76 (3H, s), 4.37 (2H, t), 6.47-6.53 (1H, m), 6.89 (1H, s), 7.44 (1H, d), 7.61 (1H, d), 7.89 (1H, s), 8.76 (1H, s), 13.13 (1H, s) |
| B38 | 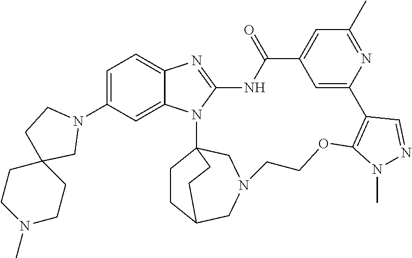 [SMILES: Cc1cc2cc(n1)-c1cnn(C)c1OCCN1CC3CCC(CC3)(C1)n1c(nc3ccc(N4CCC5(CCN(C)CC5)C4)cc31)NC2=O] | 650.45 | ¹H NMR (DMSO-d₆, 400 MHz) δ 1.50- 1.65 (4H, m), 1.83 (2H, t), 1.88-2.01 (4H, m), 2.03-2.12 (3H, m), 2.20 (3H, s), 2.35 (4H, s), 2.54 (3H, s), 2.81 (2H, d), 2.86 (2H, t), 3.09 (2H, s), 3.28-3.30 (2H, m), 3.46 (4H, d), 3.76 (3H, s), 4.37 (2H, t), 6.44-6.55 (1H, m), 6.88 (1H, s), 7.44 (1H, d), 7.61 (1H, s), 7.89 (1H, s), 8.75 (1H, s), 13.14 (1H, s). |
| B39 | 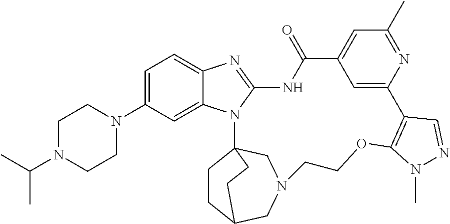 [SMILES: Cc1cc2cc(n1)-c1cnn(C)c1OCCN1CC3CCC(CC3)(C1)n1c(nc3ccc(N4CCN(C(C)C)CC4)cc31)NC2=O] | 624.35 | 1H NMR (CD₃OD, 400 MHz) 8 1.32 (6H, d), 1.88-2.01 (4H, m), 2.03-2.12 (3H, m), 2.49 (3H, s), 2.81 (2H, d), 2.86 (2H, t), 3.20 (4H, t), 3.28-3.36 (4H, m), 3.42-3.50 (5H, m), 3.72 (3H, s), 4.29 (2H, t), 6.92-6.97 (1H, m), 7.34 (1H, d), 7.44 (1H, d), 7.62 (1H, s), 7.94 (1H, s), 8.79 (1H, s). |

### Example B40A and Example B40B

### 5-[4-(dimethylamino)piperidin-1-yl]-15,21-dimethyl-23-oxa-2,9,11,16,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,15,17(33),18(22),19-nonaen-12-one (Isomer 1, Example B40A)

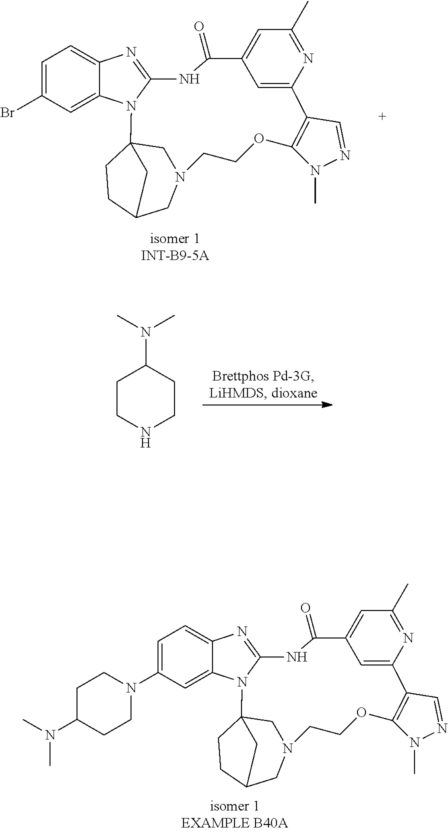

To a stirred mixture of 5-bromo-15,21-dimethyl-23-oxa-2,9,11,16,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,15,17(33),18(22),19-nonaen-12-one (isomer 1, INT-B9-5A, 80 mg, 0.142 mmol, 1 equiv), N,N-dimethylpiperidin-4-amine (54.71 mg, 0.426 mmol, 3 equiv), and BrettPhos Pd G₃ (38.68 mg, 0.043 mmol, 0.3 equiv) in 1,4-dioxane (8 mL) was added LiHMDS (1.420 mmol, 10 equiv) dropwise at room temperature under nitrogen atmosphere. The mixture was stirred for 1 h at 60 °C and then cooled to room temperature. The reaction was quenched with saturated NH₄Cl (aq.) (10 mL) and the mixture was extracted with dichloromethane (3×20 mL). The combined organic layers were washed with brine (3×30 mL), dried over anhydrous Na₂SO₄, and concentrated under reduced pressure. The residue was purified by prep-HPLC with XBridge Shield RP18 OBD column using water containing 10 mmol/L NH₄HCO₃ and 0.1% NH₃·H₂O and acetonitrile as mobile phase. Fractions containing the desired compound were evaporated to dryness to give 33.2 mg of 5-[4-(dimethylamino)piperidin-1-yl]-15,21-dimethyl-23-oxa-2,9,11,16,20,21,26-heptaazaheptacyclo[24.4.1.1\hat{ }{1,28}.1\hat{ }{13,17}.0\hat{ }{2,10}.0\hat{ }{3,8}.0\hat{ }{18,22}1]tritriaconta-3,5,7,9,13,15,17(33),18(22),19-nonaen-12-one (isomer 1, EXAMPLE B40A) as yellow solid (37%). LCMS: m/z (EST), [M+H]⁺=610.35. ¹H NMR (DMSO-d₆, 400 MHz) δ 1.65-1.78 (6H, m), 1.87-1.93 (2H, m), 1.96-2.12 (1H, d), 2.25 (6H, s), 2.45-2.49 (2H, m), 2.51-2.55 (4H, m), 2.65-2.69 (4H, m), 3.20-3.26 (2H, m), 3.35-3.45 (1H, m), 3.61-3.68 (2H, m), 3.74 (4H, s), 4.56 (1H, t), 4.70 (1H, d), 6.90-7.02 (1H, m), 7.21 (1H, d), 7.42 (1H, d), 7.56 (1H, s), 7.94 (1H, s), 8.42 (1H, s), 12.69 (1H, s). SFC-HPLC, Rt=8.811 min.

### 5-[4-(dimethylamino)piperidin-1-yl]-15,21-dimethyl-23-oxa-2,9,11,16,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,15,17(33),18(22),19-nonaen-12-one (Isomer 2, Example B40B)

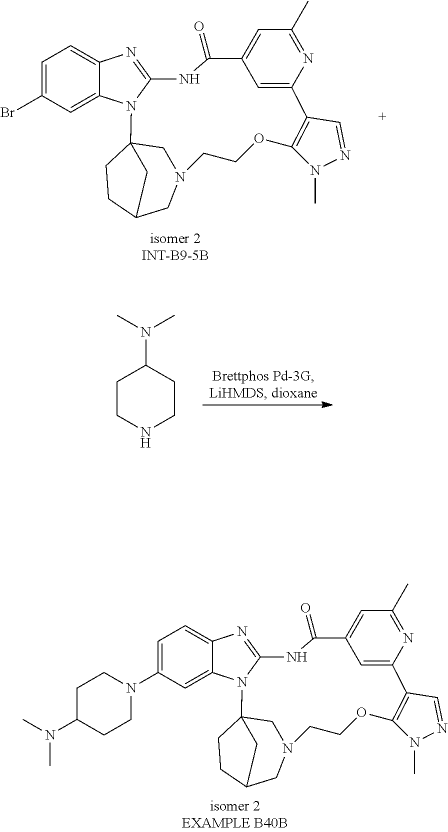

To a stirred mixture of 5-bromo-15,21-dimethyl-23-oxa-2,9,11,16,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,15,17(33),18(22),19-nonaen-12-one (isomer 2, INT-B9-5B, 80 mg, 0.14 mmol, 1 equiv) and N,N-dimethylpiperidin-4-amine (109 mg, 0.85 mmol, 6 equiv) and BrettPhos Pd G₃ (38 mg, 0.04 mmol, 0.3 equiv) in 1,4-dioxane) was added LiHMDS (3 mL, 3.00 mmol, 21.0 equiv) dropwise at room temperature under nitrogen atmosphere. The mixture stirred for 1.5 h at 60 °C under nitrogen atmosphere. The mixture was cooled to room temperature and then quenched with saturated ammonium chloride (aq.) (100 mL) at room temperature. The mixture was extracted with dichloromethane (3×150 mL). The combined organic layers were dried over anhydrous sodium sulfate and concentrated under reduced pressure.

The residue was purified by prep-TLC, eluted with dichloromethane/methanol (10/1). The product was further purified by prep-HPLC with column of XBridge Prep OBD C18 (30*150 mm, 5 μm) using mobile phase A of water (10 mmol/L NH₄HCO₃ and 0.05% NH₃H₂O) and mobile phase B of acetonitrile to give 29.7 mg of 5-[4-(dimethylamino)piperidin-1-yl]-15,21-dimethyl-23-oxa-2,9,11,16,20,21,26-heptaazaheptacyclo[24.4.1.1\hat{ }{1,28}.1\hat{ }{13,17}.0\hat{ }{2,10}.0\hat{ }{3,8}.0\hat{ }{18,22}]tritriaconta-3,5,7,9,13,15,17(33),18(22),19-nonaen-12-one (isomer 2, EXAMPLE B40B) as a yellow solid (34%). LCMS: m/z (EST), [M+H]⁺=610.30. ¹H NMR (DMSO-d₆, 400 MHz) δ 1.51-1.75 (5H, m), 1.87-1.93 (2H, m), 2.03-2.12 (2H, m), 2.26 (6H, s), 2.45-2.49 (2H, m), 2.51-2.55 (4H, m), 2.72 (4H, d), 3.20-3.26 (2H, m), 3.35-3.45 (1H, m), 3.61-3.68 (2H, m), 3.74 (4H, s), 4.52-4.60 (1H, m), 4.70 (1H, d), 6.93 (1H, d), 7.21 (1H, s), 7.42 (1H, d), 7.57 (1H, s), 7.94 (1H, s), 8.42 (1H, s), 12.70 (1H, s). SFC-HPLC, Rt=7.034, ee=100%. Rt1=3.894, Rt2=4.686.

EXAMPLE B41A, EXAMPLE B42A, EXAMPLE B43A, EXAMPLE B44A, EXAMPLE B44A1, EXAMPLE B44A2, EXAMPLE B45A, EXAMPLE B46A, EXAMPLE B47A, EXAMPLE B48A, EXAMPLE B49A, EXAMPLE B50A, EXAMPLE B51A, EXAMPLE B52A, EXAMPLE B53A, EXAMPLE B54A, EXAMPLE B55A, EXAMPLE B56A, EXAMPLE B57A, EXAMPLE B58A, EXAMPLE B59A (Table 4) were prepared using similar reaction conditions described above for the synthesis of EXAMPLE B40A from INT-9B-5A.

EXAMPLE B41B, EXAMPLE B42B, EXAMPLE B43B, EXAMPLE B44B, EXAMPLE B44B1, EXAMPLE B44B2, EXAMPLE B45B, EXAMPLE B46B, EXAMPLE B47B, EXAMPLE B48B, EXAMPLE B49B, EXAMPLE B50B, EXAMPLE B51B, EXAMPLE B52B, EXAMPLE B53B, EXAMPLE B54B, EXAMPLE B55B, EXAMPLE B56B, EXAMPLE B57B, EXAMPLE B58B, EXAMPLE B59B (Table 4) were prepared using similar reaction conditions described above for the synthesis of EXAMPLE B40B from INT-9B-5B.

TABLE 4

| Example | | [M + H] ⁺ | |
|---|---|---|---|
| No. | Structure | (observed) | ¹H NMR δ (ppm) |
| B41A | 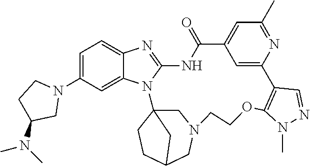 [SMILES: Cc1cc2cc(n1)-c1cnn(C)c1OCCN1CC3CCC(C3)(C1)n1c(nc3ccc(N4CC[C@H](N(C)C)C4)cc31)NC2=O] | 596.30 | ¹H NMR (DMSO-d₆, 400 MHz) δ 1.56- 1.98 (4H, m), 2.02 (1H, d), 2.20-2.24 (1H, m), 2.25 (6H, s), 2.44-2.48 (3H, m), 2.53 (3H, s), 2.55-2.59 (1H, m), 2.71-2.79 (2H, m), 2.80-2.90 (1H, m), 3.10 (1H, t), 3.19 (2H, d), 3.30-3.37 (3H, m), 3.74-3.82 (4H, m), 4.56 (1H, t), 4.72 (1H, d), 6.50-6.54 (1H, m), 6.77 (1H, s), 7.39 (1H, d), 7.56 (1H, d), 7.94 (1H, s), 8.42 (1H, s), 12.62 (1H, s). |
| | isomer 1 | | |
| B41B | 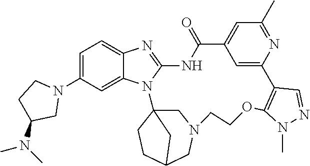 [SMILES: Cc1cc2cc(n1)-c1cnn(C)c1OCCN1CC3CCC(C3)(C1)n1c(nc3ccc(N4CC[C@H](N(C)C)C4)cc31)NC2=O] | 596.25 | ¹H NMR (DMSO-d₆, 400 MHz) δ 1.59- 1.79 (3H, m), 1.85 (1H, q), 2.02 (1H, d), 2.20-2.24 (1H, m), 2.25 (6H, s), 2.44-2.48 (3H, m), 2.53 (3H, s), 2.55-2.59 (1H, m), 2.71-2.79 (2H, m), 2.80-2.90 (1H, m), 3.10 (1H, t), 3.19 (2H, d), 3.30-3.37 (3H, m), 3.74-3.82 (4H, m), 4.56 (1H, t), 4.71 (1H, d), 6.44-6.58 (1H, m), 6.78 (1H, s), 7.39 (1H, d), 7.56 (1H, s), 7.94 (1H, s), 8.42 (1H, s), 12.60 (1H, s). |
| | isomer 2 | | |
| B42A | 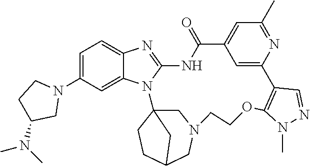 [SMILES: Cc1cc2cc(n1)-c1cnn(C)c1OCCN1CC3CCC(C3)(C1)n1c(nc3ccc(N4CC[C@@H](N(C)C)C4)cc31)NC2=O] | 596.30 | ¹H NMR (DMSO-d₆, 400 MHz) δ 1.60- 1.78 (3H, m), 1.81-1.99 (1H, m), 2.02 (1H, d), 2.20-2.24 (1H, m), 2.25 (6H, s), 2.55 (3H, s), 2.74-2.86 (4H, m), 2.88-2.89 (2H, m), 2.92-2.95 (1H, m), 3.04-3.11 (1H, m), 3.38-3.45 (2H, m), 3.53-3.59 (3H, m), 3.68-377 (4H, m), 4.56 (1H, t), 4.71 (1H, d), 6.52 (1H, d), 6.75-6.79 (1H, m), 7.39 (1H, d), 7.56 (1H, d), 7.94 (1H, s), 8.42 (1H, s), 12.62 (1H, s). |
| | isomer 1 | | |
| B42B | 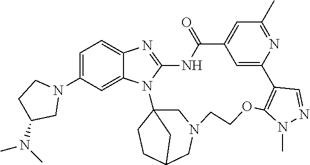 [SMILES: Cc1cc2cc(n1)-c1cnn(C)c1OCCN1CC3CCC(C3)(C1)n1c(nc3ccc(N4CC[C@@H](N(C)C)C4)cc31)NC2=O] | 596.30 | ¹H NMR (DMSO-d₆, 400 MHz) δ 1.60- 1.99 (5H, m), 2.02 (1H, d), 2.20-2.24 (2H, m), 2.25 (6H, s), 2.55 (3H, s), 2.56-2.66 (2H, m), 2.74-2.86 (2H, m), 2.88-2.89 (1H, m), 3.04-3.11 (1H, m), 3.23-3.26 (1H, m), 3.41-3.50 (3H, m), 3.74 (5H, s), 4.56 (1H, t), 4.71 (1H, d), 6.52 (1H, d), 6.77 (1H, s), 7.39 (1H, d), 7.56 (1H, s), 7.94 (1H, s), 8.42 (1H, s), 12.62 (1H, s). |
| | isomer 2 | | |
| B43A | 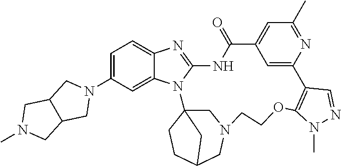 [SMILES: Cc1cc2cc(n1)-c1cnn(C)c1OCCN1CC3CCC(C3)(C1)n1c(nc3ccc(N4CC5CN(C)CC5C4)cc31)NC2=O] | 608.25 | ¹H NMR (DMSO-d₆, 400 MHz) δ 1.68- 1.83 (3H, m), 2.02 (1H, d), 2.24 (3H, s), 2.38-2.48 (4H, m), 2.57 (3H, s), 2.70-2.75 (6H, m), 2.82-2.88 (2H, m), 2.95-2.98 (2H, m), 3.01-3.15 (2H, m), 3.20-3.50 (2H, m), 3.55-3.59 (4H,m), 4.56 (1H, t), 4.71 (1H, d), 6.60-6.66 (1H, m), 6.85-6.92 (1H, m), 7.39 (1H, d), 7.56 (1H, d), 7.94 (1H, s), 8.42 (1H, s), 12.64 (1H, s). |
| | isomer 1 | | |
| B43B | 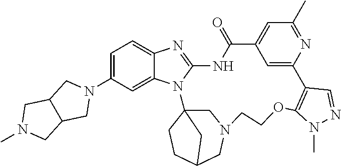 [SMILES: Cc1cc2cc(n1)-c1cnn(C)c1OCCN1CC3CCC(C3)(C1)n1c(nc3ccc(N4CC5CN(C)CC5C4)cc31)NC2=O] | 608.50 | ¹H NMR (DMSO-d₆, 400 MHz) δ 1.56- 1.65 (1H, m), 1.66-1.81 (2H, m), 2.03 (1H, d), 2.23 (3H, s), 2.38-2.45 (4H, m), 2.57 (3H, s), 2.58-2.64 (4H, m), 2.65-2.80 (2H, m), 2.83-2.96 (2H, m), 3.11 (2H, t), 3.23 (1H, d), 3.34-3.47 (3H, m), 3.73 (4H, s), 4.56 (1H, t), 4.71 (1H, d), 6.60-6.65 (1H, m), 6.90 (1H, d), 7.39 (1H, d), 7.56 (1H, s), 7.94 (1H, s), 8.42 (1H, s), 12.61 (1H, s). |
| | isomer 2 | | |
| B44A | 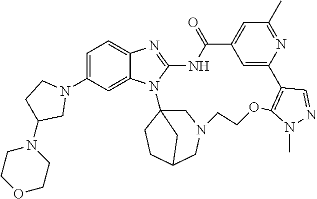 [SMILES: Cc1cc2cc(n1)-c1cnn(C)c1OCCN1CC3CCC(C3)(C1)n1c(nc3ccc(N4CCC(N5CCOCC5)C4)cc31)NC2=O] | 638.30 | ¹H NMR (DMSO-d₆, 400 MHz) δ 1.59- 1.67 (3H, m), 1.78-1.82 (1H, m), 2.02 (1H, d), 2.14-2.24 (1H, m), 2.29-2.45 (6H, m), 2.55 (3H, s), 2.56-2.62 (2H, m), 2.69- 2.79 (2H, m), 2.94 (1H, t), 3.11 (1H, q), 3.32-3.39 (2H, m), 3.42-3.46 (3H, m), 3.54 -3.56 (4H, m), 3.74-3.79 (4H, m), 4.56 (1H, t), 4.71 (1H, d), 6.50-6.57 (1H, m), 6.78 (1H, s), 7.39 (1H, d), 7.56 (1H, d), 7.94 (1H, s), 8.42 (1H, s), 12.61 (1H, s). |
| | mixture 1 | | |
| B44A1 | 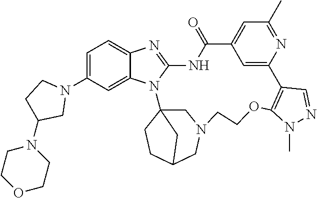 [SMILES: Cc1cc2cc(n1)-c1cnn(C)c1OCCN1CC3CCC(C3)(C1)n1c(nc3ccc(N4CCC(N5CCOCC5)C4)cc31)NC2=O] | 638.35 | ¹H NMR (DMSO-d₆, 400 MHz) δ 1.57 (1H, s), 1.65-1.69 (1H, m), 1.72-1.75 (1H, m), 1.84-1.87 (1H, m), 2.02 (1H, d), 2.20- 2.23 (1H, m), 2.35-2.40 (4H, m), 2.45 (3H, s), 2.54 (4H, d), 2.57 (2H, d), 2.67 (1H, s), 2.81 (1H, s), 3.12 (2H, t), 3.26- 3.29 (3H, m), 3.38-3.40 (4H, m), 3.46- 3.50 (4H, m), 4.56 (1H, t), 4.71 (1H, d), 6.52 (1H, d), 6.77 (1H, d), 7.39 (1H, d), 7.56 (1H, s), 7.94 (1H, s), 8.42 (1H, s), 12.55 (1H, s). |
| | isomer 1 | | |
| B44A2 | 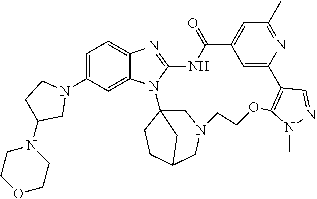 [SMILES: Cc1cc2cc(n1)-c1cnn(C)c1OCCN1CC3CCC(C3)(C1)n1c(nc3ccc(N4CCC(N5CCOCC5)C4)cc31)NC2=O] | 638.35 | ¹H NMR (DMSO-d₆, 400 MHz) δ 1.61- 1.75 (3H, m), 1.85-1.91 (1H, m), 2.02 (1H, d), 2.14-2.24 (1H, m), 2.45-2.47 (4H, m), 2.55 (3H, s), 2.56-2.61 (4H, m), 2.62- 2.80 (2H, m), 2.93 (1H, t), 3.09 (1H, t), 3.25 (2H, q), 3.50 (1H, t), 3.62 (4H, t), 3.74 (4H, s), 4.56 (1H, t), 4.70 (1H, d), 6.52 (1H, d), 6.78 (1H, d), 7.39 (1H, d), 7.56 (1H, s), 7.94 (1H, s), 8.42 (1H, s), 12.62 (1H, s). |
| | isomer 2 | | |
| B44B | 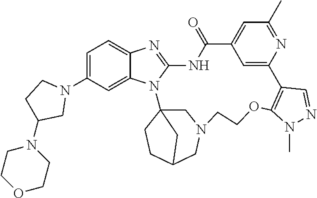 [SMILES: Cc1cc2cc(n1)-c1cnn(C)c1OCCN1CC3CCC(C3)(C1)n1c(nc3ccc(N4CCC(N5CCOCC5)C4)cc31)NC2=O] | 638.60 | ¹H NMR (DMSO-d₆, 400 MHz) δ 1.57- 1.75 (3H, m), 1.84 (1H, t), 2.02 (1H, d), 2.15-2.25 (1H, m), 2.29-2.45 (6H, m), 2.55 (3H, s), 2.56-2.62 (3H, m), 2.65-2.80 (2H, m), 2.90-2.99 (1H, m), 3.11 (1H, q), 3.22-3.26(1H, m), 3.39-3.52 (3H, m), 3.62 (4H, t), 3.73 (4H, s), 4.56 (1H, t), 4.71 (1H, d), 6.41-6.60 (1H, m), 6.78 (1H, s), 7.39 (1H, d), 7.56 (1H, s), 7.94 (1H, s), 8.42 (1H, s), 12.13-12.89 (1H, m). |
| | mixture 2 | | |
| B44B1 | 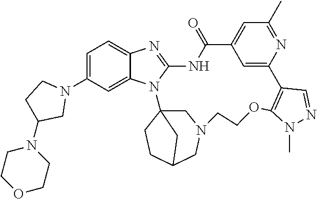 [SMILES: Cc1cc2cc(n1)-c1cnn(C)c1OCCN1CC3CCC(C3)(C1)n1c(nc3ccc(N4CCC(N5CCOCC5)C4)cc31)NC2=O] | 638.35 | ¹H NMR (DMSO-d₆, 400 MHz) δ 1.55- 1.91 (4H, m), 2.02 (1H, d), 2.19 (1H, s), 2.29-2.45 (3H, m), 2.55 (3H, s), 2.56-2.62 (3H, m), 2.65-2.80 (2H, m), 2.95 (1H, q), 3.09 (1H, t), 3.25 (3H, q), 3.37-3.53 (4H, m), 3.62 (4H, t), 3.74 (4H, s), 4.56 (1H, t), 4.70 (1H, d), 6.49-6.55 (1H, m), 6.78 (1H, d), 7.39 (1H, d), 7.56 (1H, s), 7.94 (1H, s), 8.42 (1H, s), 12.58 (1H, s). |
| | isomer 3 | | |
| B44B2 | 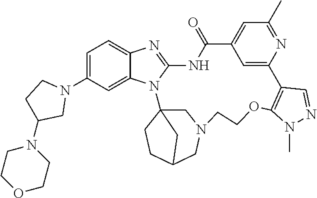 [SMILES: Cc1cc2cc(n1)-c1cnn(C)c1OCCN1CC3CCC(C3)(C1)n1c(nc3ccc(N4CCC(N5CCOCC5)C4)cc31)NC2=O] | 638.35 | ¹H NMR (DMSO-d₆, 400 MHz) δ 1.55- 1.91 (5H, m), 2.02 (1H, d), 2.19 (1H, s), 2.29-2.45 (3H, m), 2.55 (3H, s), 2.56-2.62 (3H, m), 2.65-2.80 (2H, m), 2.95 (1H, q), 3.09 (1H, t), 3.25 (3H, q), 3.37-3.53 (4H, m), 3.62 (4H, t), 3.74 (4H, s), 4.56 (1H, t), 4.70 (1H, d), 6.49-6.55 (1H, m), 6.78 (1H, d), 7.40 (1H, d), 7.56 (1H, s), 7.93 (1H, s), 8.42 (1H, s), 12.55 (1H, s). |
| | isomer 4 | | |
| B45A | 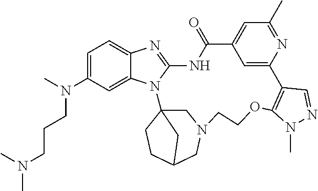 [SMILES: Cc1cc2cc(n1)-c1cnn(C)c1OCCN1CC3CCC(C3)(C1)n1c(nc3ccc(N(C)CCCN(C)C)cc31)NC2=O] | 598.30 | ¹H NMR (DMSO-d₆, 400 MHz) δ 1.60- 1.78 (5H, m), 2.03 (1H, d), 2.18 (6H, s), 2.22-2.32 (2H, m), 2.43-2.49 (3H, m), 2.53 (3H, s), 2.54-2.60 (3H, m), 2.68-2.80 (2H, m), 2.89 (3H, s), 3.20-3.28 (1H, m), 3.40-3.46 (1H, m), 3.74 (4H, s), 4.56 (1H, t), 4.71 (1H, d), 6.68-6.73 (1H, m), 7.00 (1H, d), 7.38 (1H, d), 7.56 (1H, s), 7.94 (1H, s), 8.42 (1H, s), 12.61 (1H, s). |
| | isomer 1 | | |
| B45B | 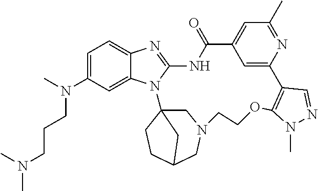 [SMILES: Cc1cc2cc(n1)-c1cnn(C)c1OCCN1CC3CCC(C3)(C1)n1c(nc3ccc(N(C)CCCN(C)C)cc31)NC2=O] | 598.40 | ¹H NMR (DMSO-d₆, 400 MHz) δ 1.56- 1.79 (5H, m), 2.03 (1H, d), 2.15 (6H, s), 2.22-2.32 (2H, m), 2.43-2.49 (3H, m), 2.53 (3H, s), 2.54-2.60 (3H, m), 2.68-2.80 (2H, m), 2.89 (3H, s), 3.20-3.28 (1H, m), 3.40-3.46 (1H, m), 3.73 (4H, s), 4.56 (1H, t), 4.71 (1H, d), 6.67-6.76 (1H, m), 7.00 (1H, d), 7.38 (1H, d), 7.56 (1H, d), 7.94 (1H, s), 8.42 (1H, s), 12.56 (1H, s). |
| | isomer 2 | | |
| B46A | 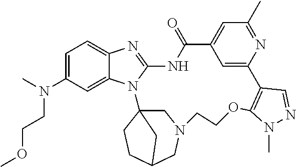 [SMILES: COCCN(C)c1ccc2nc3n(c2c1)C12CCC(CN(CCOc4c(cnn4C)-c4cc(cc(C)n4)C(=O)N3)C1)C2] | 571.35 | ¹H NMR (DMSO-d₆, 400 MHz) δ 1.60- 1.78 (3H, m), 2.03 (1H, d), 2.40-2.46 (2H, m), 2.55 (3H, s), 2.62-2.80 (2H, m), 2.94 (3H, s), 3.27 (3H, s), 3.20-3.31 (3H, m), 3.37-3.56 (5H, m), 3.74 (3H, s), 3.75-3.79 (1H, m), 4.56 (1H, t), 4.71 (1H, d), 6.71 (1H, s), 6.94-7.02 (1H, m), 7.38 (1H, d), 7.56 (1H, s), 7.94 (1H, s), 8.42 (1H, s), 12.62 (1H, s). |
| | isomer 1 | | |
| B46B | 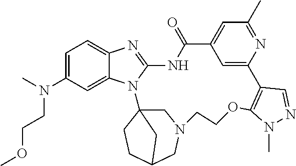 [SMILES: COCCN(C)c1ccc2nc3n(c2c1)C12CCC(CN(CCOc4c(cnn4C)-c4cc(cc(C)n4)C(=O)N3)C1)C2] | 571.30 | ¹H NMR (DMSO-d₆, 400 MHz) δ1.56- 1.78 (3H, m), 2.03 (1H, d), 2.40-2.46 (2H, m), 2.55 (3H, s), 2.62-2.80 (2H, m), 2.94 (3H, s), 3.27 (3H, s), 3.20-3.31 (3H, m), 3.37-3.56 (5H, m), 3.74 (3H, s), 3.75-3.79 (1H, m), 4.56 (1H, t), 4.71 (1H, d), 6.63- 6.75 (1H, m), 7.01 (1H, d), 7.38 (1H, d), 7.56 (1H, s), 7.94 (1H, s), 8.43 (1H, s), 12.60 (1H, s). |
| | isomer 2 | | |
| B47A | 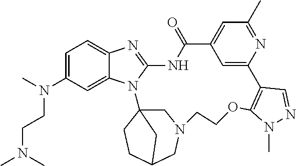 [SMILES: Cc1cc2cc(n1)-c1cnn(C)c1OCCN1CC3CCC(C3)(C1)n1c(nc3ccc(N(C)CCN(C)C)cc31)NC2=O] | 584.30 | ¹H NMR (DMSO-d₆, 400 MHz) δ 1.59- 1.68 (4H, m), 2.03 (1H, d), 2.21 (6H, s), 2.35-2.46 (4H, m), 2.53 (3H, s), 2.54-2.59 (1H, m), 2.68-2.80 (2H, m), 2.93 (3H, s), 3.18-3.28 (1H, m), 3.34-3.38 (3H, m), 3.73 (3H, s), 3.74-3.76 (1H, m), 4.56 (1H, t), 4.71 (1H, d), 6.65-6.72 (1H, m), 6.97 (1H, s), 7.39 (1H, d), 7.56 (1H, s), 7.94 (1H, s), 8.42 (1H, s), 12.61 (1H, s). |
| | isomer 1 | | |
| B47B | 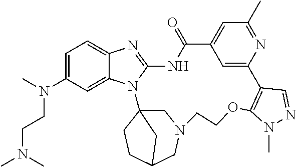 [SMILES: Cc1cc2cc(n1)-c1cnn(C)c1OCCN1CC3CCC(C3)(C1)n1c(nc3ccc(N(C)CCN(C)C)cc31)NC2=O] | 584.30 | ¹H NMR (DMSO-d₆, 400 MHz) δ 1.57- 1.81 (4H, m), 2.03 (1H, d), 2.20 (6H, s), 2.37-2.45 (4H, m), 2.53 (3H, s), 2.54-2.59 (1H, m), 2.68-2.80 (2H, m), 2.93 (3H, s), 3.18-3.28 (1H, m), 3.34-3.38 (3H, m), 3.73 (3H, s), 3.74-3.76 (1H, m), 4.56 (1H, t), 4.71 (1H, d), 6.66-6.73 (1H, m), 6.97 (1H, d), 7.39 (1H, d), 7.56 (1H, d), 7.94 (1H, s), 8.42 (1H, s), 12.59 (1H, s). |
| | isomer 2 | | |
| B48A | 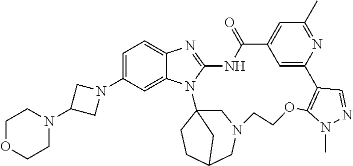 [SMILES: Cc1cc2cc(n1)-c1cnn(C)c1OCCN1CC3CCC(C3)(C1)n1c(nc3ccc(N4CC(N5CCOCC5)C4)cc31)NC2=O] | 624.30 | ¹H NMR (DMSO-d₆, 400 MHz) δ 1.61- 1.75 (3H, m), 2.01 (1H, d), 2.35 (4H, s), 2.40-2.48 (3H, m), 2.55 (4H, s), 2.65-2.80 (2H, m), 3.20-3.28 (2H, m), 3.34-3.50 (1H, m), 3.41-3.45 (6H, m), 3.72 (3H, s), 3.74-3.78 (1H, m), 3.88-4.02 (2H, m), 4.56 (1H, t), 4.70 (1H, d), 6.37 (1H, dd), 6.68 (1H, d), 7.40 (1H, d), 7.56 (1H, d), 7.94 (1H, s), 8.42 (1H, s), 12.66 (1H, s). |
| | isomer 1 | | |
| B48B | 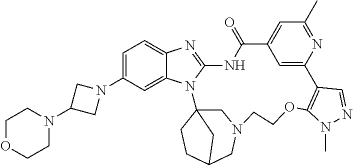 [SMILES: Cc1cc2cc(n1)-c1cnn(C)c1OCCN1CC3CCC(C3)(C1)n1c(nc3ccc(N4CC(N5CCOCC5)C4)cc31)NC2=O] | 624.30 | ¹H NMR (DMSO-d₆, 400 MHz) δ 1.57- 1.79 (3H, m), 2.03 (1H, d), 2.37 (4H, s), 2.40-2.48 (2H, m), 2.55 (4H, s), 2.65-2.80 (2H, m), 3.20-3.28 (2H, m), 3.34-3.50 (2H, m), 3.41-3.45 (6H, m), 3.72 (3H, s), 3.74-3.78 (1H, m), 3.88-4.02 (2H, m), 4.57 (1H, t), 4.71 (1H, d), 6.26-6.51 (1H, m), 6.69 (1H, s), 7.41 (1H, d), 7.57 (1H, s), 7.95 (1H, s), 8.43 (1H, s), 12.64 (1H, s). |
| | isomer 2 | | |
| B49A | 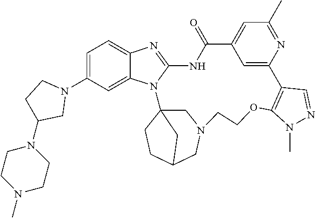 [SMILES: Cc1cc2cc(n1)-c1cnn(C)c1OCCN1CC3CCC(C3)(C1)n1c(nc3ccc(N4CCC(N5CCN(C)CC5)C4)cc31)NC2=O] | 651.35 | ¹H NMR (DMSO-d₆, 400 MHz) δ 1.70- 1.73 (4H, m), 2.02 (1H, d), 2.15 (3H, s), 2.15-2.20 (2H, m), 2.22-2.43 (3H, m), 2.45 (4H, t), 2.55 (2H, s), 2.52-2.62 (5H, m), 2.68-2.80 (2H, m), 2.92 (1H, q), 3.06- 3.09 (1H, m), 3.21-3.27 (2H, m), 3.40- 3.48 (3H, m), 3.73 (3H, s), 3.74-3.78 (1H, m), 4.56 (1H, t), 4.71 (1H, d), 6.50-6.54 (1H, m), 6.77 (1H, s), 7.39 (1H, d), 7.56 (1H, d), 7.94 (1H, s), 8.42 (1H, s), 12.61 (1H, s). |
| | isomer 1 | | |
| B49B | 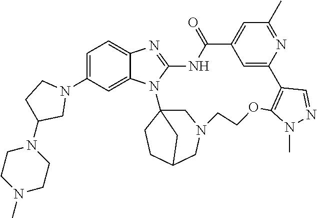 [SMILES: Cc1cc2cc(n1)-c1cnn(C)c1OCCN1CC3CCC(C3)(C1)n1c(nc3ccc(N4CCC(N5CCN(C)CC5)C4)cc31)NC2=O] | 651.25 | ¹H NMR (DMSO-d₆, 400 MHz) δ 1.55- 1.90 (5H, m), 2.03 (1H, d), 2.15 (3H, s), 2.15-2.20 (2H, m), 2.25-2.39 (3H, m), 2.40-2.48 (4H, m), 2.51-2.61 (6H, m), 2.70-2.78 (2H, m), 2.88-2.94 (1H, m), 3.06-3.12 (1H, m), 3.20-3.24 (1H, m), 3.40-3.48 (4H, m), 3.73 (3H, s), 3.74-3.78 (1H, m), 4.56 (1H, t), 4.71 (1H, d), 6.51 (1H, dd), 6.77 (1H, s), 7.39 (1H, d), 7.56 (1H, d), 7.94 (1H, s), 8.42 (1H, s), 12.58 (1H, s). |
| | isomer 2 | | |
| B50A | 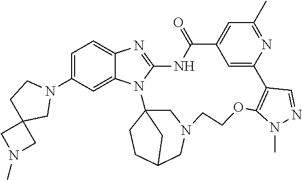 [SMILES: Cc1cc2cc(n1)-c1cnn(C)c1OCCN1CC3CCC(C3)(C1)n1c(nc3ccc(N4CCC5(CN(C)C5)C4)cc31)NC2=O] | 608.40 | ¹H NMR (DMSO-d₆, 400 MHz) δ 1.67- 1.78 (3H, m), 1.92 (1H, s), 2.02 (2H, d), 2.13-2.20 (3H, m), 2.24 (2H, s), 2.40-2.60 (6H, m), 2.70-2.81 (2H, m), 3.14 (4H, s), 3.20-3.28 (3H, m), 3.33-3.45 (3H, m), 3.73 (3H, s), 3.74-3.78 (1H, m), 4.56 (1H, t), 4.71 (1H, d), 6.45-6.51 (1H, m), 6.77 (1H, s), 7.39 (1H, d), 7.56 (1H, s), 7.94 (1H, s), 8.42 (1H, s), 12.62 (1H, s). |
| | isomer 1 | | |
| B50B | 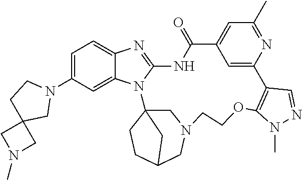 [SMILES: Cc1cc2cc(n1)-c1cnn(C)c1OCCN1CC3CCC(C3)(C1)n1c(nc3ccc(N4CCC5(CN(C)C5)C4)cc31)NC2=O] | 608.45 | ¹H NMR (DMSO-d₆, 400 MHz) δ 1.63- 1.69 (3H, m), 1.92 (1H, s), 2.02 (2H, d), 2.13-2.20 (3H, m), 2.24 (2H, s), 2.40-2.60 (6H, m), 2.70-2.81 (2H, m), 3.14 (4H, s), 3.20-3.28 (3H, m), 3.33-3.45 (3H, m), 3.73 (3H, s), 3.74-3.78 (1H, m), 4.55 (1H, d), 4.71 (1H, d), 6.49 (1H, d), 6.77 (1H, s), 7.39 (1H, d), 7.56 (1H, s), 7.94 (1H, s), 8.42 (1H, s), 12.56 (1H, s). |
| | isomer 2 | | |
| B51A | 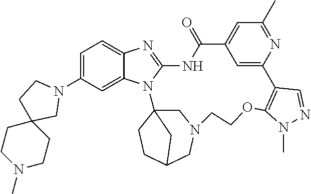 [SMILES: Cc1cc2cc(n1)-c1cnn(C)c1OCCN1CC3CCC(C3)(C1)n1c(nc3ccc(N4CCC5(CCN(C)CC5)C4)cc31)NC2=O] | 636.35 | ¹H NMR (DMSO-d₆, 400 MHz) δ 1.64- 1.79 (7H, m), 1.84 (2H, t), 2.02 (1H, d), 2.18 (3H, s), 2.28-2.38 (4H, m), 2.40-2.46 (2H, m), 2.52-2.67 (6H, m), 2.70-2.80 (2H, m), 3.04-3.12 (2H, m), 3.23-3.29 (2H, m), 3.38-3.46 (1H, m), 3.73 (3H, s), 3.74-3.78 (1H, m), 4.56 (1H, t), 4.71 (1H, d), 6.45-6.52 (1H, m), 6.75 (1H, d), 7.38 (1H, d), 7.56 (1H, d), 7.94 (1H, s), 8.42 (1H, s), 12.61 (1H, s). |
| | isomer 1 | | |
| B51B | 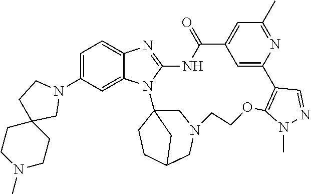 [SMILES: Cc1cc2cc(n1)-c1cnn(C)c1OCCN1CC3CCC(C3)(C1)n1c(nc3ccc(N4CCC5(CCN(C)CC5)C4)cc31)NC2=O] | 636.45 | ¹H NMR (DMSO-d₆, 400 MHz) δ 1.60- 1.70 (7H, m), 1.82-1.86 (2H, t), 2.03 (1H, d), 2.19 (3H, s), 2.28-2.38 (4H, m), 2.40- 2.46 (2H, m), 2.52-2.67 (6H, m), 2.70- 2.80 (2H, m), 3.04-3.12 (2H, m), 3.23- 3.29 (2H, m), 3.38-3.46 (1H, m), 3.73 (3H, s), 3.74-3.78 (1H, m), 4.56 (1H, t), 4.71 (1H, d), 6.49 (1H, d), 6.75 (1H, s), 7.38 (1H, d), 7.56 (1H, s), 7.94 (1H, s), 8.42 (1H, s), 12.58 (1H, s). |
| | isomer 2 | | |
| B52A | 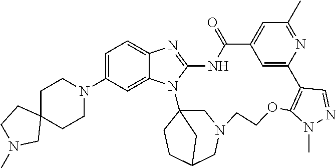 [SMILES: Cc1cc2cc(n1)-c1cnn(C)c1OCCN1CC3CCC(C3)(C1)n1c(nc3ccc(N4CCC5(CCN(C)C5)CC4)cc31)NC2=O] | 636.40 | ¹H NMR (DMSO-d₆, 400 MHz) δ 1.58- 1.85 (9H, m), 2.03 (1H, d), 2.28 (3H, s), 2.40 (2H, s), 2.44-2.48 (2H, m), 2.53 (3H, s), 2.56-2.66 (3H, m), 2.68-2.81 (2H, m), 3.00-3.14 (4H, m), 3.20-3.27 (2H, m), 3.35-3.45 (1H, m), 3.73 (3H, s), 3.74-3.78 (1H, m), 4.60 (1H, t), 4.74 (1H, d), 6.96 (1H, d), 7.23 (1H, d), 7.45 (1H, d), 7.60 (1H, d), 7.97 (1H, s), 8.46 (1H, s), 12.69 (1H, s). |
| | isomer 1 | | |
| B52B | 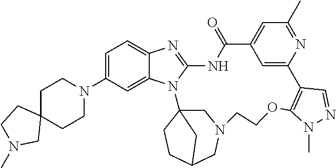 [SMILES: Cc1cc2cc(n1)-c1cnn(C)c1OCCN1CC3CCC(C3)(C1)n1c(nc3ccc(N4CCC5(CCN(C)C5)CC4)cc31)NC2=O] | 636.40 | ¹H NMR (DMSO-d₆, 400 MHz) δ 1.56- 1.77 (9H, m), 2.03 (1H, d), 2.22 (3H, s), 2.33 (2H, s), 2.44-2.48 (2H, m), 2.53 (3H, s), 2.56-2.66 (3H, m), 2.68-2.81 (2H, m), 3.00-3.14 (4H, m), 3.20-3.27 (2H, m), 3.35-3.45 (1H, m), 3.73 (3H, s), 3.74-3.78 (1H, m), 4.56 (1H, t), 4.70 (1H, d), 6.93 (1H, d), 7.20 (1H, s), 7.39-7.43 (1H, m), 7.57 (1H, s), 7.94 (1H, s), 8.42 (1H, s), 12.69 (1H, s). |
| | isomer 2 | | |
| B53A | 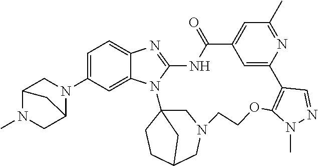 [SMILES: Cc1cc2cc(n1)-c1cnn(C)c1OCCN1CC3CCC(C3)(C1)n1c(nc3ccc(N4CC5CC4CN5C)cc31)NC2=O] | 594.40 | ¹H NMR (DMSO-d₆, 400 MHz) δ 1.60- 1.77 (4H, m), 1.83-1.91 (1H, m), 2.04 (1H, d), 2.26 (3H, s), 2.39-2.47 (4H, m), 2.53 (3H, s), 2.55-2.70 (3H, m), 2.71-2.85 (2H, m), 3.17 (1H, d), 3.20-3.28 (1H, m), 3.32-3.48 (2H, m), 3.73 (3H, s), 3.74-3.78 (1H, m), 4.32 (1H, s), 4.56 (1H, t), 4.69 (1H, d), 6.50-6.58 (1H, m), 6.80 (1H, s), 7.37 (1H, d), 7.56 (1H, d), 7.94 (1H, s), 8.42 (1H, s), 12.60 (1H, s). |
| | isomer 1 | | |
| B53B | 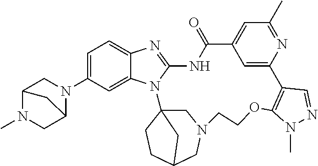 [SMILES: Cc1cc2cc(n1)-c1cnn(C)c1OCCN1CC3CCC(C3)(C1)n1c(nc3ccc(N4CC5CC4CN5C)cc31)NC2=O] | 594.35 | ¹H NMR (DMSO-d₆, 400 MHz) δ 1.55- 1.81 (4H, m), 1.83-1.91 (1H, m), 2.03 (1H, d), 2.26 (3H, s), 2.38-2.46 (3H, m), 2.53 (3H, s), 2.55-2.70 (2H, m), 2.71-2.85 (3H, m), 3.12-3.18 (2H, m), 3.35-3.45 (3H, m), 3.73 (3H, s), 3.74-3.78 (1H, m), 4.28 (1H, s), 4.56 (1H, t), 4.71 (1H, d), 6.54 (1H, dd), 6.78 (1H, d), 7.37 (1H, d), 7.56 (1H, d), 7.94 (1H, s), 8.42 (1H, s), 12.56 (1H, s). |
| | isomer 2 | | |
| B54A | 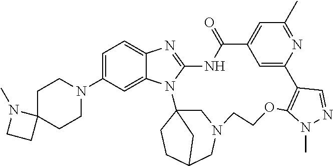 [SMILES: Cc1cc2cc(n1)-c1cnn(C)c1OCCN1CC3CCC(C3)(C1)n1c(nc3ccc(N4CCC5(CC4)CCN5C)cc31)NC2=O] | 622.35 | ¹H NMR (DMSO-d₆, 400 MHz) δ 1.54- 1.78 (5H, m), 1.81 (2H, d), 1.90 (2H, t), 2.03 (1H, d), 2.15 (3H, s), 2.40-2.46 (2H, m), 2.53 (3H, s), 2.55-2.59 (1H, m), 2.61- 2.77 (4H, m), 3.08-3.14 (2H, m), 3.22- 3.46 (3H, m), 3.52 (2H, t), 3.73 (3H, s), 3.74-3.78 (1H, m), 4.56 (1H, t), 4.65-4.78 (1H, m), 6.92-7.02 (1H, m), 7.21 (1H, d), 7.41 (1H, d), 7.56 (1H, d), 7.94 (1H, s), 8.42 (1H, s), 12.69 (1H, s). |
| | isomer 1 | | |
| B54B | 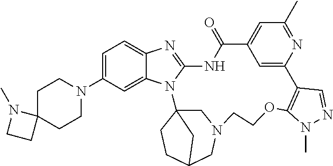 [SMILES: Cc1cc2cc(n1)-c1cnn(C)c1OCCN1CC3CCC(C3)(C1)n1c(nc3ccc(N4CCC5(CC4)CCN5C)cc31)NC2=O] | 622.35 | ¹H NMR (DMSO-d₆, 400 MHz) δ 1.58- 1.83 (7H, m), 1.90 (2H, t), 2.03 (1H, d), 2.13 (3H, s), 2.40-2.46 (2H, m), 2.53 (3H, s), 2.55-2.59 (1H, m), 2.61-2.77 (4H, m), 3.08-3.14 (2H, m), 3.22-3.46(3H, m), 3.47-3.56 (2H, m), 3.73 (3H, s), 3.74-3.78 (1H, m), 4.56 (1H, t), 4.70 (1H, d), 6.92- 7.02 (1H, m), 7.21 (1H, d), 7.41 (1H, d), 7.56 (1H, d), 7.94 (1H, s), 8.42 (1H, s), 12.69 (1H, s). |
| | isomer 2 | | |
| B55A | 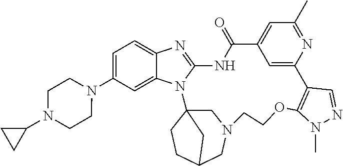 [SMILES: Cc1cc2cc(n1)-c1cnn(C)c1OCCN1CC3CCC(C3)(C1)n1c(nc3ccc(N4CCN(C5CC5)CC4)cc31)NC2=O] | 608.40 | ¹H NMR (DMSO-d₆, 400 MHz) δ 0.28- 0.37 (2H, m), 0.40-0.49 (2H, m), 1.56- 1.75 (4H, m), 2.03 (1H, d), 2.40-2.46 (2H, m), 2.53 (3H, s), 2.55-2.59 (2H, m), 2.65- 2.80 (6H, m), 2.99-3.14 (4H, m), 3.21- 3.28 (1H, m), 3.38-3.50 (1H, m), 3.73 (3H, s), 3.74-3.78 (1H, m), 4.56 (1H, t), 4.71 (1H, d), 6.94 (1H, dd), 7.19 (1H, d), 7.43 (1H, d), 7.57 (1H, d), 7.94 (1H, s), 8.42 (1H, s), 12.69 (1H, s). |
| | isomer 1 | | |
| B55B | 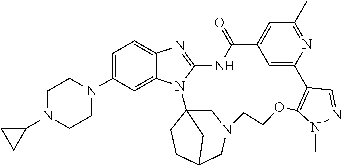 [SMILES: Cc1cc2cc(n1)-c1cnn(C)c1OCCN1CC3CCC(C3)(C1)n1c(nc3ccc(N4CCN(C5CC5)CC4)cc31)NC2=O] | 608.40 | ¹H NMR (DMSO-d₆, 400 MHz) δ 0.28- 0.37 (2H, m), 0.40-0.49 (2H, m), 1.51- 1.83 (4H, m), 2.03 (1H, d), 2.40-2.46 (2H, m), 2.53 (3H, s), 2.55-2.59 (2H, m), 2.65- 2.80 (6H, m), 2.99-3.14 (4H, m), 3.21- 3.28 (1H, m), 3.38-3.50 (1H, m), 3.73 (3H, s), 3.74-3.78 (1H, m), 4.56 (1H, t), 4.70 (1H, d), 6.92 (1H, dd), 7.19 (1H, d), 7.42 (1H, d), 7.56 (1H, d), 7.94 (1H, s), 8.42 (1H, s), 12.67 (1H, s). |
| | isomer 2 | | |
| B56A | 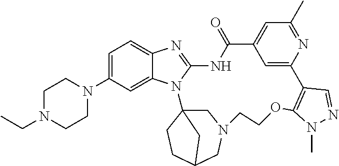 [SMILES: CCN1CCN(c2ccc3nc4n(c3c2)C23CCC(CN(CCOc5c(cnn5C)-c5cc(cc(C)n5)C(=O)N4)C2)C3)CC1] | 596.30 | ¹H NMR (DMSO-d₆, 400 MHz) δ 1.05 (3H, t), 1.58-1.75 (3H, m), 2.03 (1H, d), 2.36-2.45 (5H, m), 2.50-2.55 (8H, m), 2.66-2.75 (2H, m), 3.11 (4H, d), 3.20-3.26 (1H, m), 3.34-3.40 (1H, m), 3.74 (3H, s), 3.75-3.78 (1H, m), 4.56 (1H, t), 4.71 (1H, d), 6.92 (1H, dd), 7.19 (1H, d), 7.44 (1H, d), 7.56 (1H, s), 7.94 (1H, s), 8.43 (1H, s), 12.69 (1H, s). |
| | isomer 1 | | |
| B56B | 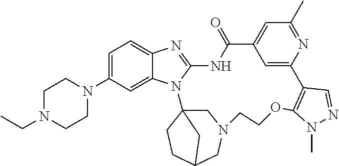 [SMILES: CCN1CCN(c2ccc3nc4n(c3c2)C23CCC(CN(CCOc5c(cnn5C)-c5cc(cc(C)n5)C(=O)N4)C2)C3)CC1] | 596.35 | ¹H NMR (DMSO-d₆, 400 MHz) δ 1.05 (3H, t), 1.51-1.84 (3H, m), 2.03 (1H, d), 2.36-2.45 (4H, m), 2.50-2.55 (9H, m), 2.64-2.78 (2H, m), 3.12 (4H, d), 3.20-3.26 (1H, d), 3.34-3.40 (1H, m), 3.74 (3H, s), 3.75-3.78 (1H, m), 4.56 (1H, t), 4.71 (1H, d), 6.92 (1H, dd), 7.20 (1H, d), 7.43 (1H, d), 7.56 (1H, s), 7.94 (1H, s), 8.43 (1H, s), 12.67 (1H, s). |
| | isomer 2 | | |
| B57A | 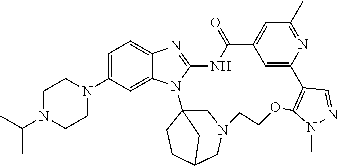 [SMILES: Cc1cc2cc(n1)-c1cnn(C)c1OCCN1CC3CCC(C3)(C1)n1c(nc3ccc(N4CCN(C(C)C)CC4)cc31)NC2=O] | 610.40 | ¹H NMR (DMSO-d₆, 400 MHz) δ 1.03 (6H, d), 1.55-1.81 (3H, m), 2.03 (1H, d), 2.40-2.48 (2H, m), 2.53 (3H, s), 2.54-2.65 (6H, m), 2.66-2.79 (3H, m), 3.10 (4H, d), 3.20-3.26 (1H, m), 3.38-3.47 (1H, m), 3.73 (3H, s), 3.75-3.78 (1H, m), 4.56 (1H, t), 4.71 (1H, d), 6.92 (1H, d), 7.20 (1H, d), 7.43 (1H, d), 7.56 (1H, s), 7.94 (1H, s), 8.43 (1H, s), 12.67 (1H, s). |
| | isomer 1 | | |
| B57B | 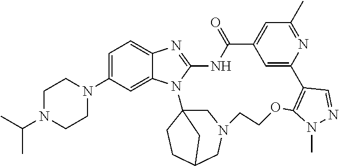 [SMILES: Cc1cc2cc(n1)-c1cnn(C)c1OCCN1CC3CCC(C3)(C1)n1c(nc3ccc(N4CCN(C(C)C)CC4)cc31)NC2=O] | 610.65 | ¹H NMR (DMSO-d₆, 400 MHz) δ 1.02 (6H, d), 1.56-1.78 (3H, m), 2.03 (1H, d), 2.40-2.48 (2H, m), 2.53 (3H, s), 2.54-2.65 (6H, m), 2.66-2.78 (3H, m), 3.10 (4H, d), 3.20-3.26 (1H, m), 3.38-3.47(1H, m), 3.73 (3H, s), 3.75-3.78 (1H, m), 4.56 (1H, t), 4.70 (1H, d), 6.93 (1H, d), 7.19 (1H, d), 7.43 (1H, d), 7.57 (1H, s), 7.94 (1H, s), 8.43 (1H, s), 12.70 (1H, s). |
| | isomer 2 | | |
| B58A | 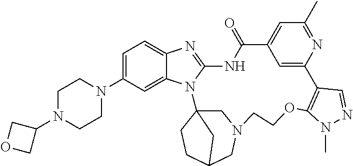 [SMILES: Cc1cc2cc(n1)-c1cnn(C)c1OCCN1CC3CCC(C3)(C1)n1c(nc3ccc(N4CCN(C5COC5)CC4)cc31)NC2=O] | 624.30 | ¹H NMR (DMSO-d₆, 400 MHz) δ 1.51- 1.89 (3H, m), 2.03 (1H, d), 2.44 (6H, t), 2.52-2.56 (5H, m), 2.66-2.79 (2H, m), 3.12-3.17 (4H, m), 3.20-3.26 (1H, m), 3.41-3.51 (2H, m), 3.73 (3H, s), 3.75-3.78 (1H, m), 4.45-4.61 (5H, m), 4.71 (1H, d), 6.89-7.04 (1H, m), 7.20 (1H, s), 7.43 (1H, d), 7.57 (1H, s), 7.94 (1H, s), 8.43 (1H, s), 12.71 (1H, s). |
| | isomer 1 | | |
| B58B | 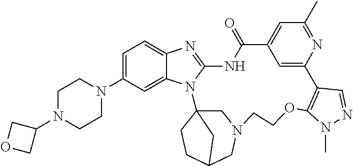 [SMILES: Cc1cc2cc(n1)-c1cnn(C)c1OCCN1CC3CCC(C3)(C1)n1c(nc3ccc(N4CCN(C5COC5)CC4)cc31)NC2=O] | 624.30 | ¹H NMR (DMSO-d₆, 400 MHz) δ 1.55- 1.82 (3H, m), 2.03 (1H, d), 2.44 (6H, t), 2.52-2.56 (5H, m), 2.66-2.79 (2H, m), 3.12-3.17 (4H, m), 3.20-3.26 (1H, m), 3.41-3.51 (2H, m), 3.73 (3H, s), 3.75-3.78 (1H, m), 4.45-4.61 (5H, m), 4.71 (1H, d), 6.89-7.04 (1H, m), 7.21 (1H, s), 7.44 (1H, d), 7.57 (1H, s), 7.95 (1H, s), 8.43 (1H, s), 12.71 (1H, s). |
| | isomer 2 | | |
| B59A | 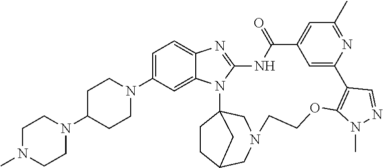 [SMILES: Cc1cc2cc(n1)-c1cnn(C)c1OCCN1CC3CCC(C3)(C1)n1c(nc3ccc(N4CCC(N5CCN(C)CC5)CC4)cc31)NC2=O] | 665.40 | ¹H NMR (DMSO-d₆, 400 MHz) δ 1.48- 1.82 (5H, m), 1.88 (2H, d), 2.04 (1H, d), 2.17 (3H, s), 2.26-2.47 (7H, m), 2.53 (3H, s), 2.55-2.60 (4H, m), 2.65-2.83 (5H, m), 3.20-3.26 (2H, m), 3.55-3.68 (3H, m), 3.73 (3H, s), 3.75-3.78 (1H, m), 4.57 (1H, t), 4.71 (1H, d), 6.93 (1H, d), 7.21 (1H, s), 7.42 (1H, d), 7.57 (1H, s), 7.95 (1H, s), 8.43 (1H, s), 12.67 (1H, s). |
| | isomer 1 | | |
| B59B | 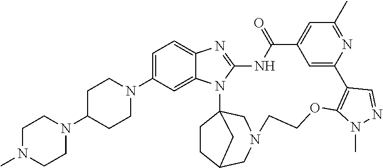 [SMILES: Cc1cc2cc(n1)-c1cnn(C)c1OCCN1CC3CCC(C3)(C1)n1c(nc3ccc(N4CCC(N5CCN(C)CC5)CC4)cc31)NC2=O] | 665.40 | ¹H NMR (DMSO-d₆, 400 MHz) δ 1.49- 1.77 (5H, m), 1.87 (2H, d), 2.03 (1H, d), 2.16 (3H, s), 2.22-2.40 (4H, m), 2.40-2.46 (2H, m), 2.53 (3H, s), 2.55-2.60 (2H, m), 2.64-2.80 (5H, m), 3.17 (2H, s), 3.20-3.26 (2H, m), 3.55-3.68 (3H, m), 3.73 (3H, s), 3.75-3.78 (1H, m), 4.10 (1H, s), 4.56 (1H, t), 4.70 (1H, d), 6.92 (1H, d), 7.20 (1H, s), 7.41 (1H, d), 7.56 (1H, s), 7.94 (1H, s), 8.42 (1H, s), 12.69 (1H, s). |
| | isomer 2 | | |

### BIOLOGICAL EXAMPLES

Exemplary compounds disclosed herein have been characterized in one or more of the following biological assays.

### Biological Example 1: Inhibition of EGFR WT and Mutant Biochemical Enzymatic Activity

Recombinant EGFR wild type, L858R, L858R/T790M, Exon19Del, Exon19Del/T790M, EGFR[T790M/C797S/L858R] and EGFR[T790M/C797S/d746-750] was purchased from Carna Bio. EGFR (d746-750 C797S), and EGFR (C797S L858R) was purchased from signalchem.

The inhibition potency of compounds against these enzymes was assessed using Homogenous Time Resolved Fluorescence approach (HTRF, CisBio. Cat No. 62TKOPEJ).

Inhibitory effects of the compounds were determined by measuring the enzymatic activity of EGFR enzyme phosphorylates 1 μM TK Substrate-biotin in the presence of 1 mM adenosine-5′-triphosphate (ATP) and varying concentrations of the test compound. The enzyme reaction buffer contains 5 mM MgCl2, 1 mM MnC12, 0.01% CHAPS, 1 mM dithiothreitol (DTT), 0.5% dimethylsulfoxide (DMSO), and 1× supplemented enzymatic buffer. The WT and mutant EGFR enzymes (SignalChem) were added to plates containing inhibitors, followed by activation of the kinase reaction by addition of ATP and peptide substrates. After incubation for 1 h at room temperature, the reaction was stopped by the addition of the detection reagent mix containing EDTA. The fluorescence was measured at 615 nm and 665 nm, respectively with excitation wavelength at 320 nm. The calculated signal ratio of 665 nm/615 nm is proportional to the kinase activity. The concentration of compound producing 50% inhibition of the respective kinase (IC₅₀) was calculated using four-parameter logistic fit with XL-FIT. Data obtained by these assays is listed in Table 5.

TABLE 5
IC₅₀ for EGFR inhibition of exemplary compounds

| | | | | | | | | IC₅₀ | IC₅₀ |
|---|---|---|---|---|---|---|---|---|---|
| | | | IC₅₀ | | IC₅₀ | IC₅₀ | IC₅₀ | (nM) | (nM) |
| | IC₅₀ | IC₅₀ | (nM) | IC₅₀ | (nM) | (nM) | (nM) | @T790M - | @T790M- |
| Example | (nM) | (nM) | @L858R- | (nM) | @Del19- | @Del19- | @L858- | C797S - | C797S - |
| No. | @WT | @L858R | T790M | @Del19 | T790M | C797S | C797S | Del19 | L858R |
| A1 | 3549 | 197 | 120 | 81 | 47 | 53 | 120 | 26 | 121 |
| A2 | 52 | 5.0 | 4.3 | 3.5 | 4.8 | 3.7 | 4.1 | 3.7 | 3.4 |
| A3 | 104 | 13 | 12 | 12 | 9.3 | 6.7 | 10 | 8.9 | 8.5 |
| A5 | >5000 | >5000 | >5000 | 849 | 704 | 628 | >5000 | 560 | >5000 |
| A6 | >5000 | >5000 | >5000 | 1672 | 1529 | 1866 | >5000 | 1229 | >5000 |
| A8 | >5000 | >5000 | >5000 | 368 | 290 | 256 | 3553 | 226 | >5000 |
| A9 | >5000 | >5000 | >5000 | 264 | 198 | 536 | >5000 | 316 | >5000 |
| A10 | >5000 | 2147 | 2628 | 189 | 164 | 173 | 1955 | 166 | 4400 |
| A12 | >5000 | 482 | 476 | 144 | 89 | 119 | 389 | 105 | 486 |
| A13 | >5000 | >5000 | >5000 | 932 | 757 | 780 | >5000 | 728 | >5000 |
| A14 | >5000 | >5000 | >5000 | 144 | 132 | 63 | >5000 | 59 | >5000 |
| A16A | >5000 | >5000 | >5000 | 150 | 127 | 152 | 1352 | 115 | >5000 |
| A16B | 288 | 28 | 22 | 5.5 | 12 | 3.2 | 16 | 10 | 14 |
| A18 | >5000 | 1371 | 1137 | 54 | 41 | 91 | 929 | 44 | 1719 |
| A19 | >5000 | 625 | 632 | 22 | 29 | 19 | 283 | 16 | 214 |
| A20 | >5000 | 301 | 82 | 86 | 41 | 42 | 132 | 40 | 76 |
| A21 | 446 | 37 | 18 | 4.6 | 7.3 | 3.7 | 15 | 7.1 | 7.2 |
| A22 | 1199 | 86 | 6.3 | 9.1 | 6.2 | 6.5 | 25 | 6.5 | 5.8 |
| A23 | >5000 | 207 | 12 | 4.7 | 3.0 | 3.0 | 61 | 2.7 | 2.7 |
| M3 | >5000 | 2464 | 2188 | 204 | 27 | 60 | 1697 | 19 | 2466 |
| A25 | 323 | 24 | 2.0 | 1.6 | 1.6 | 1.0 | 6.0 | 1.6 | 1.6 |
| A26 | 1086 | 68 | 19 | 8.0 | 5.0 | 2.9 | 33 | 5.6 | 16 |
| A27 | 290 | 25 | 26 | 6.1 | 15 | 5.5 | 21 | 13 | 20 |
| A28 | 4144 | 174 | 137 | 21 | 22 | 14 | 93 | 21 | 100 |
| A29 | 109 | 14 | 15 | 3.8 | 7.6 | 2.1 | 7.9 | 8.2 | 8.9 |
| A33 | 22 | 7.3 | 5.8 | 3.1 | 3.4 | 2.1 | 6.0 | 3.1 | 5.2 |
| A35 | 2.7 | 1.5 | 2.2 | 1.1 | 1.9 | 0.84 | 1.8 | 2.0 | 2.0 |
| A36 | 193 | 30 | 8.0 | 3.0 | 2.5 | 2.1 | 14 | 2.4 | 4.9 |
| A37 | 281 | 40 | 6.5 | 3.4 | 1.4 | 1.7 | 13 | 1.2 | 2.3 |
| A38 | 159 | 9.7 | 2.7 | 1.3 | 1.6 | 0.81 | 2.9 | 1.7 | 1.7 |
| A39 | 1517 | 74 | 14 | 5.9 | 5.0 | 2.8 | 30 | 5.1 | 8.2 |
| A40 | 71 | 11 | 6.1 | 4.4 | 4.5 | 2.3 | 6.7 | 4.3 | 5.2 |
| A41 | 74 | 10 | 5.1 | 2.3 | 3.5 | 1.7 | 5.3 | 3.1 | 3.2 |
| A42 | 42 | 8.9 | 6.8 | 3.0 | 5.4 | 2.5 | 6.7 | 4.5 | 5.1 |
| A43 | 295 | 21 | 13 | 4.5 | 3.7 | 2.2 | 8.9 | 4.4 | 7.1 |
| A44 | 236 | 34 | 15 | 4.9 | 3.3 | 3.0 | 15 | 3.1 | 8.8 |
| A46 | 104 | 14 | 8.5 | 2.6 | 4.1 | 2.2 | 8.5 | 3.9 | 5.7 |
| A47 | 1125 | 46 | 9.5 | 4.5 | 4.2 | 2.5 | 20 | 4.2 | 4.9 |
| A48 | 2247 | 40 | 5.0 | 3.1 | 2.8 | 2.1 | 28 | 3.1 | 4.4 |
| A49 | 694 | 45 | 8.1 | 5.5 | 6.0 | 2.8 | 16 | 4.9 | 5.1 |
| A50 | 124 | 15 | 6.7 | 2.4 | 3.8 | 1.8 | 7.4 | 4.0 | 4.8 |
| A51 | 1830 | 94 | 33 | 13 | 12 | 4.7 | 31 | 12 | 24 |
| A52 | 54 | 6.3 | 6.6 | 1.3 | 3.5 | 1.4 | 4.7 | 3.4 | 3.9 |
| A53 | >5000 | 462 | 70 | 15 | 12 | 7.2 | 135 | 13 | 21 |
| A54 | >5000 | 3043 | 213 | 118 | 31 | 41 | 887 | 31 | 10 |
| A55 | 103 | 16 | 8.4 | 3.7 | 3.7 | 1.8 | 8.4 | 4.4 | 7.2 |
| A56 | 501 | 35 | 32 | 9.1 | 17 | 5.5 | 22 | 16 | 19 |
| A57 | >5000 | >5000 | 4532 | 189 | 112 | 175 | 2646 | 65 | >5000 |
| A58 | 220 | 26 | 34 | 9.2 | 16 | 4.9 | 19 | 15 | 22 |
| A59 | >5000 | >5000 | 2614 | 202 | 11 | 140 | 4718 | 11 | 807 |
| A60 | >5000 | 917 | 240 | 23 | 8.6 | 18 | 619 | 9.4 | 157 |
| A60A | >5000 | >5000 | >5000 | 276 | 141 | 196 | >5000 | 117 | >5000 |
| A60B | 4481 | 135 | 49 | 3.2 | 0.82 | 1.7 | 65 | 0.81 | 44 |
| A61A | >5000 | >5000 | >5000 | 2179 | >5000 | 1664 | >5000 | >5000 | >5000 |
| A61B | 2417 | 224 | 8.0 | 6.1 | 1.8 | 2.5 | 146 | 1.7 | 17 |
| A62 | 2040 | 144 | 7.6 | 9.6 | 6.2 | 3.8 | 63 | 6.3 | 7.3 |
| A62B | 809 | 45 | 6.3 | 3.5 | 2.8 | 1.4 | 19 | 2.9 | 4.3 |
| A63B | 8.0 | 1.6 | 0.92 | 0.86 | 0.94 | 0.51 | 1.1 | 1.1 | 0.96 |
| A64A | >5000 | >5000 | >5000 | 2161 | >5000 | 1916 | >5000 | 2097 | >5000 |
| A64B | 3.6 | 2.0 | 2.0 | 1.0 | 1.8 | 0.95 | 2.0 | 2.0 | 1.9 |
| A65B | 148 | 16 | 2.4 | 0.94 | 1.7 | 0.97 | 7.4 | 1.7 | 1.8 |
| A66B | 62 | 7.9 | 2.6 | 1.9 | 2.8 | 1.7 | 8.1 | 2.9 | 3.2 |
| A66D | 18 | 3.7 | 1.7 | 2.7 | 2.0 | 1.1 | 3.7 | 1.6 | 2.1 |
| A67A | >5000 | >5000 | >5000 | 1019 | 4071 | 1080 | >5000 | 1641 | >5000 |
| A67B | 2.1 | 1.1 | 1.1 | 0.65 | 1.3 | 0.48 | 1.2 | 1.2 | 1.1 |
| A68B | 8.9 | 2.9 | 1.8 | 1.2 | 1.8 | 0.95 | 3.1 | 1.8 | 1.8 |
| A69A | >5000 | >5000 | >5000 | 1205 | >5000 | 1780 | >5000 | >5000 | >5000 |
| A69B | 291 | 22 | 2.8 | 2.9 | 2.0 | 1.2 | 14 | 2.2 | 3.0 |
| A70B | 19 | 4.7 | 1.8 | 1.6 | 1.9 | 0.91 | 3.6 | 2.1 | 2.1 |
| A71B | 319 | 14 | 2.8 | 1.8 | 2.5 | 1.3 | 20 | 2.4 | 2.9 |
| A72B | 5.2 | 2.4 | 1.8 | 1.8 | 2.4 | 0.92 | 2.0 | 2.2 | 1.3 |
| A73B | 13 | 4.0 | 1.9 | 1.3 | 2.2 | 1.4 | 2.9 | 2.1 | 1.9 |
| A74 | >5000 | 707 | 81 | 24 | 8.6 | 23 | 573 | 6.8 | 367 |
| A75 | 672 | 44 | 6.0 | 3.3 | 3.8 | 2.8 | 35 | 3.4 | 8.0 |
| A76A | >5000 | 2859 | >5000 | 450 | >5000 | 503 | 4298 | 4855 | >5000 |
| A76B | 206 | 39 | 7.0 | 4.2 | 4.9 | 3.5 | 27 | 4.4 | 6.9 |
| A77A | >5000 | 1111 | >5000 | 456 | >5000 | 464 | >5000 | >5000 | >5000 |
| A77B | 7.3 | 3.5 | 3.8 | 2.9 | 3.8 | 2.1 | 3.7 | 3.6 | 3.4 |
| A78A | >5000 | 2037 | >5000 | 508 | >5000 | 494 | >5000 | 3750 | >5000 |
| A78B | 11 | 2.4 | 2.4 | 1.2 | 2.7 | 0.91 | 2.2 | 1.6 | 1.4 |
| A79A | >5000 | >5000 | >5000 | 1434 | >5000 | 1581 | >5000 | >5000 | >5000 |
| A79B | 126 | 27 | 12 | 6.3 | 10 | 5.0 | 13 | 11 | 8.3 |
| A80A2 | >5000 | >5000 | >5000 | 490 | >5000 | 530 | >5000 | 2194 | >5000 |
| A80B1 | 9.3 | 7.2 | 7.8 | 3.2 | 6.2 | 3.1 | 8.8 | 5.1 | 6.7 |
| A81A | >5000 | 1530 | >5000 | 404 | >5000 | 389 | >5000 | >5000 | >5000 |
| A81B | 6.6 | 3.2 | 2.3 | 1.8 | 2.8 | 1.7 | 3.1 | 2.3 | 2.3 |
| A82A | 341 | 1211 | >5000 | 310 | 1408 | 226 | 2144 | 629 | >5000 |
| A82B | 0.92 | 0.87 | 1.8 | 1.1 | 2.1 | 0.87 | 1.4 | 2.0 | 1.7 |
| A83A | >5000 | 2017 | >5000 | 428 | >5000 | 577 | 2597 | >5000 | >5000 |
| A83B | 83 | 5.0 | 2.8 | 0.96 | 2.7 | 0.96 | 5.0 | 2.7 | 2.3 |
| A84A | >5000 | 830 | >5000 | 226 | >5000 | 258 | 1178 | >5000 | >5000 |
| A84B | >5000 | 1309 | >5000 | 451 | >5000 | 400 | 1141 | >5000 | >5000 |
| A84C | 57 | 6.7 | 5.0 | 2.6 | 3.4 | 1.4 | 4.8 | 2.5 | 3.5 |
| A84D | 196 | 21 | 4.6 | 3.1 | 3.9 | 2.4 | 9.8 | 3.3 | 4.4 |
| A85A | >5000 | 1591 | >5000 | 138 | >5000 | 308 | 1285 | 3660 | >5000 |
| A85B | 84 | 11 | 3.1 | 1.2 | 2.2 | 1.6 | 5.5 | 2.2 | 3.1 |
| A86A | >5000 | 2118 | >5000 | 689 | >5000 | 599 | 2817 | >5000 | >5000 |
| A86B | 110 | 16 | 12 | 4.0 | 9.5 | 3.9 | 14 | 8.5 | 8.1 |
| A87A | >5000 | 4482 | >5000 | 842 | >5000 | 995 | >5000 | 4004 | >5000 |
| A87B | 36 | 9.3 | 8.8 | 2.8 | 9.5 | 4.1 | 8.9 | 8.6 | 8.7 |
| A88A | >5000 | >5000 | >5000 | 477 | >5000 | 1162 | >5000 | >5000 | >5000 |
| A88B | 12 | 4.8 | 4.9 | 2.5 | 4.9 | 2.2 | 4.2 | 4.6 | 2.8 |
| A89A | >5000 | 3597 | >5000 | 250 | >5000 | 573 | >5000 | >5000 | >5000 |
| A89B | 134 | 16 | 7.4 | 1.9 | 3.8 | 2.2 | 11 | 4.0 | 5.4 |
| A90A | >5000 | >5000 | >5000 | 1710 | >5000 | 749 | >5000 | >5000 | >5000 |
| A90B | 825 | 54 | 10.0 | 6.3 | 5.9 | 3.5 | 31 | 4.9 | 9.3 |
| A91A | >5000 | 3720 | >5000 | 740 | >5000 | 1646 | >5000 | >5000 | >5000 |
| A91B | 110 | 11 | 12 | 4.0 | 9.7 | 2.9 | 8.0 | 6.7 | 6.1 |
| A92A | >5000 | 1151 | >5000 | 573 | >5000 | 449 | 2795 | 3961 | >5000 |
| A92B | 16 | 3.2 | 2.3 | 1.5 | 2.6 | 1.0 | 2.6 | 2.3 | 2.2 |
| A94A | >5000 | 4744 | >5000 | 830 | >5000 | 1557 | >5000 | >5000 | >5000 |
| A94B | 235 | 29 | 11 | 5.2 | 11 | 6.1 | 20 | 8.4 | 9.5 |
| A94B1 | 148 | 27 | 8.8 | 10 | 7.1 | 3.7 | 15 | 5.0 | 5.0 |
| A94B2 | 289 | 45 | 9.7 | 8.9 | 6.3 | 4.5 | 30 | 5.2 | 9.7 |
| A95A | >5000 | 4638 | >5000 | 864 | >5000 | 899 | >5000 | >5000 | >5000 |
| A95B | 55 | 7.0 | 3.7 | 1.7 | 3.1 | 1.5 | 4.7 | 2.7 | 3.3 |
| A96 | >5000 | 207 | 12 | 4.7 | 3.0 | 3.0 | 61 | 2.7 | 2.7 |
| A97A | >5000 | 3026 | >5000 | 1191 | >5000 | 850 | 3954 | >5000 | >5000 |
| A97B | 224 | 14 | 5.7 | 4.4 | 4.9 | 1.7 | 8.4 | 4.8 | 4.7 |
| A98A | >5000 | 1275 | >5000 | 235 | 3409 | 391 | 3370 | 2084 | >5000 |
| A98B | 21 | 3.3 | 2.7 | 1.8 | 2.9 | 1.3 | 4.1 | 2.7 | 2.3 |
| A99B | 3532 | 94 | 11 | 3.5 | 8.4 | 4.6 | 59 | 7.7 | 10 |
| A100 | >5000 | 2501 | 3522 | 97 | 25 | 120 | 1760 | 26 | 2950 |
| A101 | >5000 | 212 | 7.4 | 3.5 | 2.3 | 2.3 | 47 | 1.8 | 3.2 |
| A102 | >5000 | 414 | 195 | 18 | 5.7 | 12 | 167 | 3.4 | 32 |
| A103 | >5000 | 168 | 65 | 7.4 | 1.8 | 5.4 | 68 | 1.3 | 20 |
| A104A | >5000 | 4683 | >5000 | 1279 | >5000 | 1393 | >5000 | >5000 | >5000 |
| A104B | 937 | 53 | 13 | 6.1 | 8.5 | 4.5 | 33 | 5.9 | 6.6 |
| A105A | >5000 | 2604 | >5000 | 381 | >5000 | 330 | 2768 | 4186 | >5000 |
| A105B | 110 | 7.3 | 1.8 | 1.6 | 2.2 | 1.0 | 3.8 | 1.7 | 1.5 |
| A106A | >5000 | 1745 | >5000 | 594 | >5000 | 446 | >5000 | 2823 | >5000 |
| A106B | 4.1 | 1.8 | 2.2 | 1.2 | 2.2 | 1.0 | 2.2 | 2.0 | 1.4 |
| A107A | >5000 | 770 | >5000 | 267 | 4499 | 245 | 1042 | 2858 | >5000 |
| A107B | 20 | 3.0 | 2.7 | 1.8 | 1.8 | 0.79 | 1.9 | 2.0 | 2.1 |
| A108A | >5000 | 577 | >5000 | 293 | >5000 | 268 | 942 | 3166 | >5000 |
| A108B | 7.7 | 1.9 | 1.3 | 1.0 | 1.7 | 0.77 | 1.5 | 1.4 | 1.1 |
| A109 | 219 | 16 | 11 | 4.7 | 7.9 | 3.1 | 9.8 | 7.6 | 10 |
| A110 | 216 | 6.2 | 5.1 | 2.8 | 2.9 | 1.7 | 4.9 | 3.2 | 6.1 |
| A111 | 607 | 36 | 86 | 21 | 46 | 18 | 40 | 47 | 63 |
| A112 | >5000 | 198 | 133 | 8.5 | 15 | 6.6 | 60 | 12 | 52 |
| A113 | >5000 | 439 | 230 | 41 | 32 | 1196 | 46 | 45 | 900 |
| A115 | >5000 | 233 | 325 | 19 | 12 | 9.2 | 112 | 6.6 | 50 |
| A116 | >5000 | >5000 | >5000 | 464 | 88 | 474 | 3698 | 168 | >5000 |
| A117B | >5000 | 463 | 1908 | 23 | 3.3 | 8.1 | 23 | 2.5 | 280 |
| A118B | 3640 | 110 | 61 | 8.7 | 7.4 | 3.2 | 37 | 2.9 | 11 |
| A119 | 1060 | 38 | 32 | 7.6 | 6.6 | 4.7 | 19 | 3.0 | 9.0 |
| A120 | >5000 | 177 | 374 | 22 | 16 | 14 | 108 | 15 | 113 |
| A121 | >5000 | 167 | 477 | 25 | 21 | 19 | 101 | 10 | 154 |
| A122 | 1418 | 48 | 39 | 6.8 | 7.6 | 3.5 | 25 | 5.2 | 8.1 |
| A123 | >5000 | 65 | 223 | 13 | 13 | 9.4 | 57 | 11 | 23 |
| A124 | 2787 | 139 | 334 | 15 | 9.8 | 11 | 76 | 7.7 | 72 |
| A125 | 4062 | 189 | 582 | 29 | 18 | 25 | 128 | 15 | 359 |
| A126 | >5000 | 95 | 169 | 13 | 12 | 9.9 | 71 | 8.9 | 52 |
| A127 | 4881 | 118 | 211 | 15 | 10 | 9.2 | 69 | 6.1 | 39 |
| A128 | >5000 | 202 | 307 | 11 | 8.0 | 4.9 | 49 | 4.7 | 33 |
| B1 | >5000 | 1483 | 15 | 150 | 6.5 | 2196 | 112 | 13 | 23 |
| B2 | >5000 | 320 | 25 | 47 | 13 | 560 | 28 | 20 | 36 |
| B3 | 954 | 24 | 7.2 | 7.0 | 4.3 | 36 | 3.1 | 7.3 | 11 |
| B4 | >5000 | >5000 | 847 | 592 | 33 | 243 | 3635 | 16 | 532 |
| B5 | >5000 | 440 | 13 | 108 | 5.1 | 48 | 195 | 4.1 | 7.1 |
| B6 | >5000 | 1093 | 9.0 | 197 | 5.2 | 112 | 390 | 3.6 | 3.1 |
| B7 | >500 | >5000 | 1959 | 1420 | 109 | 627 | >5000 | 44 | 1093 |
| B7A | >5000 | >5000 | 2634 | 1206 | 104 | 611 | >5000 | 62 | 1652 |
| B7B | >5000 | >5000 | 909 | 864 | 42 | 322 | 4430 | 17 | 529 |
| B8A | >5000 | >5000 | >5000 | 729 | 4003 | 659 | >5000 | 3989 | >5000 |
| B8B | >5000 | 469 | 1.3 | 14 | 0.95 | 4.2 | 200 | 0.98 | 1.00 |
| B9A | >5000 | >5000 | >5000 | 432 | 1074 | 454 | >5000 | 245 | >5000 |
| B9B | 114 | 7.4 | 1.1 | 1.4 | 1.3 | 0.73 | 3.3 | 1.4 | 1.0 |
| B10A | 3370 | 212 | 2.6 | 4.6 | 2.8 | 2.3 | 59 | 2.2 | 2.6 |
| B10B | >5000 | >5000 | >5000 | 1083 | >5000 | 1431 | >5000 | 4036 | >5000 |
| B11 | >5000 | >5000 | >5000 | >5000 | 510 | 4319 | >5000 | 401 | >5000 |
| B12 | >5000 | 44 | 3.9 | 3.7 | 2.5 | 2.3 | 11 | 2.6 | 3.0 |
| B13 | 2535 | 160 | 7.0 | 5.8 | 3.2 | 3.8 | 37 | 4.1 | 4.2 |
| B16 | >5000 | >5000 | 446 | 497 | 7.1 | 197 | >5000 | 3.2 | 55 |
| B17 | >5000 | >5000 | >5000 | >5000 | 128 | 2110 | >5000 | 51 | 4163 |
| B18 | >5000 | >5000 | >5000 | 942 | 33 | >500 | >5000 | 31 | 1992 |
| B20 | >5000 | >5000 | >5000 | >5000 | 685 | >5000 | >5000 | 122 | >5000 |
| B23 | >5000 | >5000 | 12 | 16 | 3.8 | 6.5 | 73 | 2.5 | 5.4 |
| B27 | 3542 | 67 | 8.3 | 11 | 8.3 | 6.3 | 30 | 3.1 | 3.8 |
| B28 | >5000 | 320 | 17 | 23 | 9.5 | 14 | 145 | 7.8 | 5.9 |
| B29 | >5000 | 352 | 28 | 45 | 17 | 28 | 204 | 8.6 | 13 |
| B30 | >5000 | 110 | 11 | 17 | 12 | 8.7 | 64 | 4.9 | 5.6 |
| B31 | >5000 | 244 | 20 | 19 | 11 | 15 | 113 | 7.3 | 8.2 |
| B32 | >5000 | 197 | 11 | 17 | 6.6 | 13 | 117 | 2.3 | 3.9 |
| B33 | >5000 | 214 | 20 | 28 | 9.9 | 22 | 131 | 3.0 | 6.2 |
| B34 | >5000 | 421 | 16 | 39 | 12 | 20 | 203 | 5.4 | 8.4 |
| B35 | >5000 | 378 | 13 | 30 | 9.0 | 14 | 185 | 6.0 | 5.0 |
| B36 | >5000 | 734 | 7.6 | 24 | 5.8 | 10 | 159 | 3.7 | 2.9 |
| B37 | >5000 | 194 | 13 | 19 | 9.0 | 13 | 99 | 3.9 | 5.6 |
| B38 | >5000 | 560 | 37 | 38 | 21 | 26 | 300 | 8.2 | 14 |
| B39 | >5000 | 282 | 7.7 | 20 | 6.6 | 9.4 | 126 | 3.3 | 4.2 |
| B40A | >5000 | >5000 | >5000 | >5000 | 430 | 2477 | >5000 | 123 | >5000 |
| B40B | 100 | 15 | 3.3 | 4.5 | 4.7 | 2.2 | 4.4 | 2.3 | 2.4 |
| B41A | >5000 | >5000 | >5000 | >5000 | 3667 | >5000 | >5000 | 1303 | >5000 |
| B41B | 581 | 34 | 4.2 | 7.5 | 4.9 | 3.0 | 8.8 | 3.3 | 2.2 |
| B42A | >5000 | >5000 | >5000 | >5000 | 2482 | >5000 | >5000 | 1083 | >5000 |
| B42B | 734 | 51 | 9.9 | 13 | 10 | 7.0 | 15 | 6.3 | 5.7 |
| B43A | >5000 | >5000 | >5000 | >5000 | 1159 | >5000 | >5000 | 200 | >5000 |
| B43B | 489 | 36 | 4.8 | 6.6 | 5.2 | 3.0 | 9.0 | 3.0 | 2.3 |
| B44A | >5000 | >5000 | >5000 | >5000 | >5000 | >5000 | >5000 | 4635 | >5000 |
| B44A1 | >5000 | >5000 | >5000 | >5000 | >5000 | >5000 | >5000 | 4533 | >5000 |
| B44A2 | >5000 | >5000 | >5000 | >5000 | >5000 | >5000 | >5000 | >5000 | >5000 |
| B44B | >5000 | 170 | 13 | 18 | 6.4 | 8.1 | 50 | 4.3 | 4.9 |
| B44B1 | >5000 | 337 | 19 | 34 | 12 | 12 | 66 | 8.8 | 6.4 |
| B44B2 | 4899 | 267 | 14 | 21 | 8.4 | 8.5 | 66 | 7.0 | 5.5 |
| B45A | >5000 | >5000 | >5000 | >5000 | 3623 | >5000 | >5000 | 1289 | >5000 |
| B45B | 1023 | 46 | 3.8 | 5.3 | 2.8 | 2.4 | 14 | 1.7 | 1.4 |
| B46A | >5000 | >5000 | >5000 | >5000 | >5000 | >5000 | >5000 | >5000 | >5000 |
| B46B | >5000 | 377 | 12 | 28 | 7.2 | 7.5 | 117 | 3.7 | 4.7 |
| B47A | >5000 | >5000 | >5000 | 4168 | 4260 | 3459 | >5000 | 1820 | >5000 |
| B47B | 805 | 59 | 3.5 | 5.8 | 2.8 | 2.8 | 14 | 1.9 | 1.5 |
| B48A | >5000 | >5000 | >5000 | 2245 | >5000 | 1022 | 2923 | >5000 | >5000 |
| B48B | 2966 | 142 | 6.7 | 13 | 6.9 | 4.5 | 44 | 3.4 | 3.0 |
| B49A | >5000 | >5000 | >5000 | >5000 | 481 | >5000 | >5000 | 289 | >5000 |
| B49B | 76 | 17 | 5.6 | 8.0 | 5.8 | 3.8 | 8.0 | 3.5 | 2.4 |
| B50A | >5000 | >5000 | >5000 | >5000 | 2285 | >5000 | >5000 | 748 | >5000 |
| B50B | 413 | 26 | 3.0 | 5.1 | 3.6 | 2.1 | 7.0 | 2.4 | 1.5 |
| B51A | >5000 | >5000 | >5000 | >5000 | 3370 | >5000 | >5000 | 1096 | >5000 |
| B51B | 546 | 21 | 5.9 | 6.1 | 5.6 | 3.5 | 8.4 | 3.4 | 2.9 |
| B52A | >5000 | >5000 | >5000 | >5000 | 100 | 2046 | >5000 | 37 | >5000 |
| B52B | 8.3 | 6.0 | 3.5 | 5.4 | 4.5 | 2.4 | 3.2 | 2.0 | 1.6 |
| B53A | >5000 | >5000 | >5000 | >5000 | 3323 | 4348 | >5000 | 1076 | >5000 |
| B53B | 971 | 53 | 2.6 | 5.2 | 3.9 | 1.9 | 9.5 | 1.9 | 1.7 |
| B54A | >5000 | >5000 | >5000 | >5000 | 647 | 4720 | >5000 | 233 | >5000 |
| B54B | 121 | 13 | 3.1 | 4.9 | 3.9 | 2.0 | 3.9 | 2.5 | 1.6 |
| B55A | >5000 | >5000 | >5000 | >5000 | >5000 | >5000 | >5000 | 2743 | >5000 |
| B55B | 1827 | 64 | 2.3 | 4.3 | 3.2 | 1.7 | 6.0 | 1.6 | 1.5 |
| B56A | >5000 | >5000 | >5000 | 3177 | 1317 | 2393 | >5000 | 360 | >5000 |
| B56B | 392 | 26 | 3.0 | 4.4 | 4.1 | 1.7 | 5.1 | 2.3 | 1.6 |
| B57A | >5000 | >5000 | >5000 | >5000 | 1766 | >5000 | >5000 | 792 | >5000 |
| B57B | 543 | 31 | 3.7 | 6.1 | 4.3 | 2.1 | 6.0 | 2.6 | 1.8 |
| B58A | >5000 | >5000 | >5000 | >5000 | >5000 | 3888 | >5000 | >5000 | >5000 |
| B58B | 2746 | 134 | 5.9 | 12 | 5.5 | 4.2 | 33 | 3.2 | 3.1 |
| B59A | >5000 | >5000 | >5000 | >5000 | 1141 | >5000 | >5000 | 514 | >5000 |
| B59B | 360 | 27 | 7.7 | 12 | 10 | 5.0 | 9.5 | 6.1 | 4.4 |

### Biological Example 2: Cell Proliferation Assays

Ba/F3 cells expressing EGFR mutations were seeded in 384-well plates at 5000 cells/well in RPMI1640 medium containing 1000 FBS. PC-9 at 500 cells/well was seeded into 384-well plates in RPMI1640 medium with 10% FBS. After overnight incubation, the assay plates were dosed with compounds at a series of concentrations. Alongside dosing the assay plates, the day 0 plate was processed using CellTiter-Glo® Luminescent Cell Viability Assay (Promega) to measure the number of viable cells (G0). The assay plates were further incubated for 72 hours and the number of viable cells (G3) was measured. The percentage of proliferation was calculated as: 00 Proliferation=100×(G3 value of sample well−G0 value)/(G3 value of DMSO control−G0 value). The concentration of compound producing 50% proliferation inhibition (GI₅₀) was further calculated in best-fit curves using XLFit software. Data obtained by these assays is listed in Table 6. N/A=not available.

TABLE 6
GI₅₀ for EGFR inhibition of exemplary compounds

| | | | | | | | L858R - | DEL19 - | |
|---|---|---|---|---|---|---|---|---|---|
| | | | L858R - | DEL19 - | L858R - | | T790- | T790- | |
| | L858R/ | | C797S/ | C797S/ | T790M/ | DEL19 - | C797S/ | C797S/ | |
| | BaF3 | PC9 | BaF3 | BaF3 | BaF3 | T790M | BaF3 | BaF3 | A-431 |
| Example | GI₅₀ | GI₅₀ | GI₅₀ | GI₅₀ | GI₅₀ | GI₅₀ | GI₅₀ | GI₅₀ | GI₅₀ |
| No. | (nM) | (nM) | (nM) | (nM) | (nM) | (nM) | (nM) | (nM) | (nM) |
| A1 | 6.1 | 68 | 21 | 22 | 60 | 114 | 27 | 170 | 3574 |
| A2 | 3.7 | 80 | 5.2 | 5.8 | 5.5 | 33 | 6.7 | 30 | 1011 |
| A3 | 9.7 | 236 | 11 | 16 | 30 | 128 | 22 | 124 | 1288 |
| A4 | 56 | 183 | 63 | 77 | 585 | 788 | 76 | 811 | 2540 |
| A5 | 454 | 482 | 764 | 392 | 961 | 747 | 859 | 1160 | 2872 |
| A6 | 1308 | 403 | 919 | 204 | 795 | 968 | 1538 | 1052 | 1782 |
| A7 | 1051 | 424 | 1155 | 279 | 803 | 768 | 976 | 830 | 1413 |
| A8 | 180 | 180 | 332 | 93 | 977 | 256 | 214 | 401 | 3885 |
| A9 | 608 | 211 | 515 | 55 | 264 | 364 | 607 | 405 | 3025 |
| A10 | 309 | 128 | 291 | 33 | 219 | 323 | 260 | 368 | 5143 |
| A11 | 2694 | 903 | 2248 | 666 | 1725 | 1794 | 2150 | 1750 | 2613 |
| A12 | 72 | 149 | 159 | 39 | 116 | 197 | 122 | 265 | 3128 |
| A13 | 234 | 292 | 232 | 148 | 411 | 192 | 280 | 192 | 357 |
| A14 | 305 | 152 | 188 | 57 | 866 | 462 | 459 | 356 | 2245 |
| A15 | 1391 | 2072 | 1575 | 495 | 1554 | 2623 | 4913 | 2104 | 10000 |
| A16A | 182 | 139 | 113 | 73 | 165 | 117 | 161 | 122 | 95 |
| A16B | 21 | 73 | 19 | 5.2 | 53 | 29 | 17 | 24 | 664 |
| A18 | 194 | 565 | 169 | 52.85 | 249 | 187 | 309 | 246 | 377 |
| A19 | 147 | 856 | 98 | 6.6 | 85 | 85 | 246 | 113 | 1345 |
| A20 | 89 | 145 | 87 | 17.13 | 39 | 226 | 51 | 204 | 10000 |
| A21 | 19 | 24 | 26 | 2.1 | 6.7 | 24 | 7.2 | 22 | 1109 |
| A22 | 30 | 87 | 22 | 9.3 | 51 | 20 | 9.6 | 11 | 2293 |
| A23 | 104 | 208 | 32 | 4.0 | 151 | 10 | 12 | 11 | 2819 |
| M3 | 257 | 252 | 344 | 89 | 982 | 300 | 159 | 364 | 1954 |
| A25 | 2.4 | 12 | 9.3 | 1.4 | 16 | 9.2 | 5.6 | 13 | 486 |
| A26 | 18 | 18 | 4.6 | 2.1 | 28 | 18 | 12 | 16 | 405 |
| A27 | 3.0 | 21 | 7.1 | 1.2 | 6.8 | 3.5 | 2.2 | 1.9 | 453 |
| A28 | 4.5 | 38 | 16 | 2.4 | 9.4 | 6.7 | 3.6 | 4.7 | 452 |
| A29 | 2.1 | 7.1 | 2.8 | 0.41 | 2.5 | 1.9 | 0.78 | 1.0 | 247 |
| A30 | 13 | 17 | 11 | 2.3 | 24 | 15 | 13 | 3.6 | 719 |
| A31 | 25 | 35 | 10 | 2.3 | 30 | 19 | 13 | 3.4 | 790 |
| A32 | 3.0 | 7.3 | 2.8 | 0.43 | 9.1 | 3.8 | 2.9 | 1.5 | 248 |
| A33 | 1.1 | 4.8 | 2.2 | 0.40 | 3.4 | 3.2 | 2.3 | 0.58 | 366 |
| A34 | 4.6 | 9.1 | 4.2 | 1.1 | 9.8 | 5.0 | 8.9 | 1.3 | 587 |
| A35 | 0.53 | 3.7 | 1.7 | 0.38 | 3.4 | 1.6 | 1.0 | 0.5 | 215 |
| A36 | 5.1 | 14 | 5.9 | 2.1 | 23 | 7.8 | 4.7 | 6.0 | 591 |
| A37 | 3.2 | 4.3 | 3.8 | 0.70 | 10 | 5.5 | 2.5 | 3.3 | 166 |
| A38 | 4.1 | 6.2 | 4.2 | 1.4 | 13 | 7.3 | 3.2 | 4.8 | 315 |
| A39 | 5.2 | 7.6 | 6.9 | 1.9 | 16 | 8.4 | 5.4 | 4.7 | 290 |
| A40 | 4.0 | 17 | 5.4 | 1.7 | 17 | 7.7 | 4.7 | 4.2 | 642 |
| A41 | 0.47 | 2.9 | 1.4 | 0.33 | 2.9 | 1.4 | 0.57 | 1.1 | 314 |
| A42 | 0.75 | 2.5 | 2.1 | 0.47 | 8.7 | 2.2 | 1.6 | 1.8 | 279 |
| A43 | 2.6 | 5.7 | 3.4 | 1.0 | 9.5 | 5.2 | 2.5 | 3.6 | 508 |
| A44 | 4.4 | 14 | 7.0 | 2.0 | 27 | 16 | 5.3 | 14 | 232 |
| A45 | 8.6 | 15 | 14 | 5.1 | 29 | 20 | 9.3 | 16 | 245 |
| A46 | 3.1 | 5.4 | 2.4 | 1.2 | 12 | 8.1 | 2.0 | 6.8 | 221 |
| A47 | 4.0 | 7.0 | 3.2 | 1.7 | 17 | 12 | 3.4 | 9.7 | 200 |
| A48 | 20 | 38 | 26 | 9.4 | 94 | 53 | 21 | 48 | 573 |
| A49 | 5.7 | 12 | 7.0 | 2.0 | 72 | 18 | 5.3 | 12 | 251 |
| A50 | 2.7 | 6.3 | 2.4 | 1 | 9.5 | 7.5 | 1.9 | 5.3 | 126 |
| A51 | 46 | 62 | 38 | 7.6 | 65 | 65 | 36 | 35 | 1040 |
| A52 | 8.1 | 15.4 | 8.4 | 1.3 | 13.1 | 9.6 | 9.6 | 6.3 | 480.1 |
| A53 | 28 | 51 | 34 | 7.4 | 51 | 49 | 35 | 27 | 639 |
| A54 | 192 | 245 | 188 | 32 | 288 | 153 | 228 | 104 | 1908 |
| A55 | 26 | 51 | 31 | 4.6 | 52 | 36 | 29 | 25 | 1278 |
| A56 | 12 | 52 | 12 | 3.4 | 26 | 17 | 10 | 11 | 436 |
| A57 | 790 | 194 | 474 | 64 | 356 | 612 | 716 | 534 | 8296 |
| A58 | 29 | 325 | 20 | 2.0 | 20 | 20 | 37 | 28 | 1748 |
| A59 | 201 | 295 | 222 | 32 | 964 | 140 | 160 | 75 | 1137 |
| A60 | 87 | 146 | 120 | 19 | 457 | 63 | 86 | 40 | 4108 |
| A60A | 315 | 633 | 519 | 96 | 1404 | 285 | 403 | 166 | 6553 |
| A60B | 21 | 15 | 15 | 1.4 | 53 | 6.9 | 8.4 | 4.4 | 1343 |
| A61A | 1369 | 1913 | 1735 | 521 | 2655 | 913 | 1906 | 688 | 5895 |
| A61B | 21 | 29 | 22 | 1.7 | 55 | 9.9 | 16 | 11 | 1788 |
| A62 | 10 | 4.9 | 9.2 | 0.66 | 4.4 | 6.1 | 9.2 | 5.3 | 2367 |
| A62A | 2150 | 1320 | 1199 | 393 | 1047 | 1402 | 2380 | 1353 | 7121 |
| A62B | 3.9 | 10 | 5.2 | 0.64 | 8.4 | 2.6 | 5.3 | 2.5 | 1071 |
| A63A | 2372 | 841 | 1025 | 324 | 3689 | 929 | 988 | 540 | 9373 |
| A63B | 1.1 | 5.0 | 3.1 | 0.67 | 8.6 | 1.9 | 1.3 | 1.4 | 620 |
| A64A | 599 | 546 | 443 | 215 | 2100 | 755 | 381 | 513 | 6127 |
| A64B | 0.91 | 3.3 | 1.7 | 0.71 | 4.9 | 1.3 | 0.79 | 0.92 | 973 |
| A65A | 945 | 710 | 561 | 209 | 1968 | 652 | 656 | 452 | 2199 |
| A65B | 2.7 | 5.6 | 3.5 | 0.71 | 11 | 2.4 | 2.8 | 2.2 | 538 |
| A66A | 1144 | 705 | 709 | 177 | 2133 | 622 | 600 | 466 | 10000 |
| A66B | 2.4 | 6.1 | 3.6 | 0.67 | 11 | 1.9 | 2.2 | 2.1 | 815 |
| A66C | 1582 | 703 | 688 | 156 | 1517 | 723 | 549 | 548 | 10000 |
| A66D | 1.7 | 4.2 | 3.2 | 0.68 | 7.2 | 2.0 | 1.4 | 1.5 | 473 |
| A67A | 530 | 307 | 382 | 133 | 1045 | 463 | 258 | 374 | 6354 |
| A67B | 0.79 | 2.5 | 1.9 | 0.64 | 3.5 | 1.0 | 0.84 | 0.92 | 712 |
| A68A | 480 | 460 | 402 | 196 | 1125 | 543 | 366 | 409 | 5319 |
| A68B | 2.1 | 3.5 | 2.8 | 0.66 | 9.6 | 1.8 | 1.3 | 1.3 | 284 |
| A69A | 643 | 723 | 598 | 227 | 1534 | 465 | 503 | 366 | 9384 |
| A69B | 2.9 | 4.9 | 3.6 | 0.62 | 10 | 2.0 | 2.0 | 1.3 | 776 |
| A70A | 603 | 554 | 452 | 188 | 1866 | 517 | 463 | 411 | 3098 |
| A70B | 0.76 | 2.7 | 2.7 | 0.66 | 5.9 | 1.6 | 0.91 | 1.0 | 744 |
| A71A | 1755 | 787 | 954 | 286 | 2408 | 735 | 762 | 579 | 7865 |
| A71B | 2.9 | 6.6 | 4.2 | 0.66 | 8.0 | 1.9 | 2.2 | 2.0 | 830 |
| A72A | 866 | 1057 | 632 | 351 | 2767 | 1227 | 1816 | 1055 | 10000 |
| A72B | 0.88 | 2.1 | 1.9 | 0.51 | 1.6 | 0.51 | 0.51 | 0.58 | 731 |
| A73A | 550 | 515 | 357 | 94 | 1103 | 190 | 184 | 302 | 10000 |
| A73B | 1.8 | 7.3 | 3.5 | 0.54 | 8.0 | 1.5 | 1.9 | 1.9 | 1072 |
| A74 | 41 | 84 | 41 | 4.5 | 104 | 37 | 60 | 40 | 2373 |
| A75 | 21 | 44 | 21 | 2.5 | 69 | 20 | 35 | 27 | 5424 |
| A76A | 536 | 810 | 304 | 272 | 942 | 584 | 980 | 564 | 7929 |
| A76B | 13 | 23 | 7.6 | 0.78 | 39 | 12 | 34 | 12 | 1636 |
| A77A | 320 | 341 | 125 | 102 | 375 | 212 | 368 | 326 | 10000 |
| A77B | 0.89 | 2.1 | 1.4 | 0.72 | 2.4 | 0.85 | 1.3 | 0.91 | 310 |
| A78A | 329 | 249 | 157 | 102 | 380 | 250 | 402 | 269 | 3047 |
| A78B | 0.87 | 2.4 | 0.74 | 0.51 | 1.0 | 0.75 | 0.90 | 0.82 | 434 |
| A79A | 990 | 993 | 360 | 251 | 1120 | 882 | 1908 | 999 | 10000 |
| A79B | 12 | 23 | 8.5 | 0.7 | 37 | 13 | 14 | 10 | 1279 |
| A80A | 312 | 295 | 193 | 135 | 450 | 477 | 523 | 587 | 10000 |
| A80A2 | 100 | 381 | 125 | 57 | 315 | 136 | 126 | 170 | 10000 |
| A80B | 0.69 | 3.3 | 0.80 | 0.69 | 2.7 | 1.2 | 2.5 | 1.3 | 483 |
| A80B1 | 1.2 | 11.0 | 3.0 | 0.55 | 4.2 | 1.0 | 1.0 | 1.0 | 6138 |
| A80B2 | 0.63 | 3.6 | 0.50 | 0.50 | 0.67 | 0.70 | 0.73 | 0.70 | 10000 |
| A81A | 319 | 247 | 170 | 107 | 350 | 178 | 381 | 258 | 1322 |
| A81B | 2.6 | 6.9 | 2.7 | 0.7 | 12 | 1.9 | 11 | 2.8 | 667 |
| A82A | 108 | 92 | 76 | 96 | 137 | 200 | 129 | 186 | 10000 |
| A82B | 0.78 | 2.4 | 0.68 | 0.77 | 0.82 | 0.9 | 1.0 | 0.86 | 203 |
| A83A | 993 | 766 | 468 | 176 | 1133 | 585 | 1089 | 789 | 7757 |
| A83B | 13 | 20 | 8.9 | 0.7 | 25 | 10 | 15 | 10 | 1187 |
| A84A | 311 | 214 | 190 | 98 | 402 | 171 | 362 | 232 | 3988 |
| A84B | 480 | 768 | 302 | 157 | 997 | 545 | 1040 | 516 | 10000 |
| A84C | 2.1 | 6.6 | 3.9 | 0.72 | 12 | 2.7 | 6.0 | 2.1 | 727 |
| A84D | 14 | 29 | 16 | 0.77 | 40 | 14 | 31 | 13 | 1767 |
| A85A | 188 | 387 | 167 | 95 | 364 | 159 | 303 | 119 | 10000 |
| A85B | 0.78 | 12 | 1.6 | 0.50 | 7.7 | 1.3 | 1.2 | 1.1 | 969 |
| A86A | 181 | 989 | 244 | 50 | 367 | 123 | 264 | 120 | 10000 |
| A86B | 2.4 | 26 | 5.2 | 0.71 | 10 | 1.2 | 4.2 | 1.9 | 2666 |
| A87A | 178 | 452 | 124 | 61 | 311 | 192 | 233 | 238 | 382 |
| A87B | 4.6 | 25 | 7.9 | 0.70 | 13 | 3.4 | 7.7 | 4.0 | 5968 |
| A88A | 119 | 611 | 100 | 57 | 361 | 163 | 252 | 159 | 6260 |
| A88B | 0.84 | 21 | 3.4 | 0.63 | 8.8 | 1.0 | 1.7 | 1.0 | 2201 |
| A89A | 135 | 791 | 165 | 103 | 367 | 115 | 280 | 178 | 6392 |
| A89B | 2.2 | 28 | 3.8 | 0.57 | 9.2 | 2.6 | 4.1 | 3.2 | 1702 |
| A90A | 540 | 2099 | 249 | 128 | 977 | 321 | 721 | 393 | 4271 |
| A90B | 4.6 | 46 | 8.0 | 0.75 | 14 | 3.2 | 15 | 3.9 | 3158 |
| A91A | 324 | 1302 | 303 | 74 | 884 | 231 | 366 | 146 | 1176 |
| A91B | 2.6 | 25 | 3.9 | 0.75 | 11 | 3.1 | 4.6 | 2.2 | 1694 |
| A92A | 113 | 522 | 122 | 102 | 310 | 80 | 152 | 112 | 2097 |
| A92B | 0.82 | 9.8 | 1.6 | 0.50 | 3.0 | 1.2 | 0.94 | 1.1 | 842 |
| A94A | 323 | 981 | 284 | 114 | 944 | 209 | 539 | 254 | 1662 |
| A94A1 | 754 | 2333 | 1797 | 936 | 2649 | 2032 | 882 | 2955 | 3545 |
| A94A2 | 715 | 1943 | 1485 | 1089 | 2711 | 2105 | 634 | 3937 | 1804 |
| A94B | 5.1 | 35 | 7.0 | 0.70 | 14 | 3.3 | 7.7 | 4.5 | 2880 |
| A94B1 | 2.0 | 16 | 6.0 | 0.73 | 12 | 3.0 | 3.9 | 6.5 | 1112 |
| A94B2 | 3.3 | 30 | 7.2 | 0.77 | 14 | 5.0 | 5.4 | 8.5 | 1980 |
| A95B | 357 | 2459 | 436 | 95 | 1082 | 177 | 574 | 228 | 10000 |
| A96 | 104 | 208 | 32 | 4.0 | 151 | 10 | 12 | 11 | 2819 |
| A97A | 129 | 763 | 325 | 102 | 1005 | 459 | 124 | 527 | 5794 |
| A97B | 2.0 | 24 | 3.0 | 0.76 | 13 | 6.4 | 3.5 | 5.6 | 1454 |
| A98A | 47 | 297 | 173 | 95 | 343 | 171 | 49 | 340 | 10000 |
| A98B | 0.54 | 7.6 | 2.8 | 0.64 | 3.0 | 1.5 | 1.1 | 1.5 | 280 |
| A99A | 372 | 3094 | 1555 | 966 | 2630 | 1246 | 874 | 2899 | 4933 |
| A99B | 3.9 | 46 | 11 | 1.2 | 39 | 7.9 | 4.6 | 15 | 3638 |
| A100 | 142 | 144 | 160 | 27 | 535 | 78 | 129 | 56 | 5997 |
| A101 | 68 | 128 | 31 | 8.1 | 260 | 92 | 57 | 58 | 1412 |
| A102 | 79 | 115 | 61 | 12 | 307 | 59 | 37 | 31 | 2909 |
| A103 | 26 | 32 | 17 | 2.7 | 72 | 18 | 10 | 7 | 1684 |
| A104A | 795 | 568 | 402 | 217 | 1005 | 473 | 675 | 503 | 1945 |
| A104B | 12 | 21 | 8.2 | 0.74 | 19 | 6.9 | 12 | 6.8 | 1012 |
| A105A | 617 | 683 | 263 | 100 | 246 | 363 | 652 | 341 | 10000 |
| A105B | 7.9 | 13 | 3.8 | 0.76 | 15 | 4.9 | 7.0 | 3.5 | 354 |
| A106A | 268 | 418 | 143 | 113 | 392 | 355 | 316 | 322 | 2499 |
| A106B | 1.1 | 7.6 | 2.2 | 0.79 | 1.1 | 1.9 | 3.0 | 1.4 | 732 |
| A107A | 240 | 238 | 105 | 66 | 332 | 183 | 235 | 160 | 2418 |
| A107B | 1.0 | 5.2 | 2.4 | 0.63 | 4.1 | 1.5 | 1.5 | 1.1 | 267 |
| A108A | 202 | 320 | 123 | 84 | 369 | 227 | 223 | 204 | 1384 |
| A108B | 1.0 | 4.6 | 1.1 | 0.67 | 3.9 | 1.3 | 1.2 | 1.0 | 320 |
| A109 | 21 | 15 | 23 | 1.7 | 34 | 16 | 27 | 15 | 489 |
| A110 | 9.6 | 112 | 14 | 2.6 | 50 | 22 | 30 | 26 | 529 |
| A111 | 103.3 | 334.0 | 124.9 | 30.3 | 657.8 | 325.4 | 309.4 | 326.5 | 8228.0 |
| A112 | 89 | 45 | 47 | 4.5 | 37 | 105 | 64 | 70 | 3586 |
| A113 | 121 | 147 | 172 | 15 | 255 | 63 | 230 | 108 | 4688 |
| A115 | 20 | 94 | 61 | 7.6 | 133 | 18 | 68 | 52 | 3037 |
| A116 | 419 | 256 | 194 | 56 | 720 | 157 | 162 | 336 | 3440 |
| A117B | 16 | 57 | 38 | 4.3 | 80 | 12 | 32 | 20 | 1512 |
| A118B | 3.8 | 16 | 8.2 | 0.68 | 19 | 1.8 | 6.7 | 4.1 | 1118 |
| A119 | 2.1 | 11 | 11 | 1.5 | 15 | 7.3 | 6.6 | 11 | 1072 |
| A120 | 8.1 | 54 | 50 | 13 | 113 | 40 | 28 | 71 | 7853 |
| A121 | 12 | 27 | 35 | 7.8 | 61 | 25 | 15 | 44 | 4973 |
| A122 | 4.1 | 22 | 19 | 3.2 | 82 | 19 | 13 | 22 | 2101 |
| A123 | 5.8 | 25 | 32 | 12 | 59 | 27 | 20 | 45 | 6110 |
| A124 | 5.2 | 27 | 32 | 7.2 | 47 | 26 | 11 | 38 | 3795 |
| A125 | 7.0 | 35 | 34 | 7.9 | 64 | 30 | 21 | 43 | 3743 |
| A126 | 5.5 | 34 | 29 | 7.3 | 50 | 26 | 18 | 36 | 4092 |
| A127 | 7.2 | 34 | 38 | 8.5 | 52 | 26 | 21 | 36 | 4676 |
| A128 | 11 | 61 | 65 | 13 | 119 | 66 | 47 | 90 | 2647 |
| B1 | 751 | 410 | 736 | 95 | 98 | 22 | 51 | 20 | 4388 |
| B2 | 125 | 102 | 153 | 18 | 14 | 5.0 | 10 | 4.7 | 3165 |
| B3 | 41 | 52 | 58 | 7.5 | 11 | 2.5 | 5.2 | 1.7 | 2024 |
| B4 | 612 | 292 | 342 | 125 | N/A | 57 | 42 | 61 | 3532 |
| B5 | 144 | 115 | 66 | 62 | 7.0 | 41 | 9.1 | 31 | 2825 |
| B6 | 35 | 145 | 69 | 62 | 16 | 48 | 7.7 | 42 | 2829 |
| B7 | 1897 | 667 | 334 | 346 | 723 | 420 | 300 | 296 | 10000 |
| B7A | 934 | 427 | 189 | 228 | 383 | 265 | 295 | 186 | 10000 |
| B7B | 1600 | 469 | 253 | 173 | 292 | 189 | 123 | 130 | 10000 |
| B8A | 3559 | 2851 | 2123 | 620 | 1172 | 298 | 290 | 251 | 6390 |
| B8B | 72 | 63 | 53 | 4.8 | 8.6 | 2.6 | 2.4 | 2.1 | 2713 |
| B9A | 471 | 438 | 386 | 83 | 253 | 135 | 46 | 107 | 10000 |
| B9B | 2.7 | 6.1 | 3.2 | 0.58 | 0.83 | 0.60 | 0.60 | 0.59 | 366.3 |
| B10A | 24 | 41 | 18 | 0.79 | 2.1 | 0.89 | 0.91 | 0.84 | 1734 |
| B10B | 2907 | 2625 | 1577 | 403 | 539 | 765 | 595 | 516 | 5516 |
| B11 | 3570 | 1492 | 2438 | 626 | 2515 | 441 | 764 | 269 | 5375 |
| B12 | 4.0 | 12 | 5.6 | 1.4 | 1.0 | 0.79 | 0.38 | 0.56 | 1085 |
| B13 | 18 | 22 | 20 | 4.8 | 3.4 | 2.9 | 1.2 | 1.0 | 507 |
| B16 | 286 | 438 | 297 | 57 | 308 | 29 | 8.8 | 19 | 3227 |
| B17 | 114 | 71 | 82 | 26 | 21 | 8.7 | 6.0 | 10 | 1089 |
| B18 | 432 | 122 | 2695 | 86 | 96 | 12 | 24 | 32 | 10000 |
| B20 | 2039 | 1284 | 10000 | 487 | 1019 | 331 | 159 | 258 | 10000 |
| B23 | 442 | 113 | 195 | 22 | N/A | 7.2 | 5.9 | 15 | 10000 |
| B27 | 17 | 19 | 22 | 2 | 5 | 2 | 2 | 2 | 3599 |
| B28 | 85 | 98 | 116 | 17 | 34 | 5 | 11 | 5 | 7811 |
| B29 | 149 | 155 | 171 | 26 | 37 | 11 | 19 | 9 | 10000 |
| B30 | 22 | 33 | 35 | 3 | 9 | 2 | 4 | 2 | 3565 |
| B31 | 81 | 103 | 127 | 24 | 38 | 10 | 19 | 10 | 1679 |
| B32 | 64 | 81 | 78 | 11 | 20 | 4 | 7 | 3 | 7406 |
| B33 | 62 | 75 | 69 | 10 | 13 | 4 | 7 | 3 | 3741 |
| B34 | 64 | 86 | 83 | 11 | 17 | 4 | 9 | 4 | 4519 |
| B35 | 62 | 121 | 108 | 10 | 20 | 3 | 7 | 3 | 6628 |
| B36 | 200 | 186 | 234 | 27 | 29 | 10 | 15 | 8 | 3418 |
| B37 | 74 | 106 | 73 | 12 | 28 | 5 | 9 | 5 | 5035 |
| B38 | 81 | 103 | 127 | 24 | 38 | 10 | 19 | 10 | 1679 |
| B39 | 167 | 178 | 101 | 24 | 18 | 7 | 4 | 8 | 7730 |
| B40A | 877 | 950 | 312 | 212 | 194 | 258 | 41 | 164 | 10000 |
| B40B | 8 | 9 | 6 | 2 | 2 | 2 | 2 | 2 | 333 |
| B41A | 2787 | 2303 | 776 | 302 | 315 | 348 | 39 | 436 | 10000 |
| B41B | 12 | 8 | 7 | 2 | 2 | 2 | 2 | 2 | 1788 |
| B42A | 1768 | 1589 | 602 | 865 | 354 | 468 | 75 | 476 | 10000 |
| B42B | 5 | 9 | 3 | 2 | 2 | 2 | 2 | 2 | 10000 |
| B43A | 998 | 1320 | 442 | 193 | 305 | 271 | 68 | 200 | 10000 |
| B43B | 12 | 7 | 7 | 2 | 2 | 2 | 2 | 2 | 437 |
| B44A | 3005 | 1400 | 624 | 312 | 326 | 368 | 52 | 321 | 10000 |
| B44A1 | 3201 | 2296 | 850 | 470 | 392 | 1214 | 114 | 454 | 10000 |
| B44A2 | 9395 | 3562 | 3818 | 873 | 1077 | 3758 | 343 | 1291 | 10000 |
| B44B | 15 | 13 | 12 | 2 | 2 | 2 | 2 | 2 | 929 |
| B44B1 | 24 | 22 | 6 | 2 | 2 | 7 | 2 | 2 | 3687 |
| B44B2 | 19 | 22 | 11 | 2 | 2 | 8 | 2 | 2 | 3062 |
| B45A | 2852 | 2797 | 10000 | 239 | 10000 | 10000 | 48 | 363 | 10000 |
| B45B | 11 | 10 | 4 | 2 | 2 | 2 | 2 | 2 | 1684 |
| B46A | 7606 | 3050 | 2499 | 977 | 919 | 497 | 198 | 492 | 7826 |
| B46B | 36 | 25 | 30 | 2 | 3 | 2 | 2 | 2 | 1356 |
| B47A | 2991 | 2264 | 721 | 371 | 380 | 417 | 97 | 500 | 10000 |
| B47B | 11 | 12 | 9 | 2 | 2 | 2 | 2 | 2 | 378 |
| B48A | 846 | 973 | 426 | 114 | 168 | 140 | 34 | 172 | 7005 |
| B48B | 13 | 12 | 6 | 2 | 2 | 2 | 2 | 2 | 1370 |
| B49A | 931 | 1478 | 750 | 325 | 353 | 550 | 57 | 606 | 10000 |
| B49B | 2 | 6 | 3 | 2 | 2 | 2 | 2 | 2 | 10000 |
| B50A | 1284 | 1547 | 741 | 302 | 320 | 377 | 52 | 437 | 10000 |
| B50B | 7 | 8 | 4 | 2 | 2 | 2 | 2 | 2 | 1573 |
| B51A | 2854 | 3025 | 1084 | 357 | 383 | 530 | 124 | 615 | 10000 |
| B51B | 8 | 8 | 4 | 2 | 2 | 2 | 2 | 2 | 3034 |
| B52A | 176 | 311 | 180 | 102 | 191 | 363 | 41 | 169 | 7015 |
| B52B | 2 | 4 | 2 | 2 | 2 | 7 | 2 | 2 | 879 |
| B53A | 2574 | 3037 | 1069 | 348 | 383 | 388 | 91 | 419 | 10000 |
| B53B | 13 | 10 | 4 | 2 | 2 | 6 | 2 | 2 | 2573 |
| B54A | 801 | 515 | 584 | 280 | 298 | 527 | 107 | 241 | 3407 |
| B54B | 2 | 3 | 2 | 2 | 2 | 6 | 2 | 2 | 795 |
| B55A | 4181 | 1929 | 2560 | 549 | 939 | 1099 | 350 | 638 | 7545 |
| B55B | 17 | 10 | 11 | 2 | 2 | 7 | 2 | 2 | 2081 |
| B56A | 1034 | 443 | 417 | 82 | 80 | 230 | 58 | 145 | 10000 |
| B56B | 12 | 10 | 5 | 2 | 2 | 7 | 2 | 2 | 426 |
| B57A | 2001 | 941 | 829 | 208 | 324 | 612 | 147 | 428 | 10000 |
| B57B | 9 | 8 | 8 | 2 | 2 | 2 | 2 | 2 | 257 |
| B58A | 1562 | 737 | 828 | 163 | 307 | 378 | 90 | 229 | 8606 |
| B58B | 13 | 9 | 9 | 2 | 2 | 2 | 2 | 2 | 532 |
| B59A | 1132 | 930 | 751 | 227 | 299 | 788 | 144 | 474 | 10000 |
| B59B | 36 | 32 | 27 | 11 | 11 | 13 | 3 | 12 | 10000 |

The foregoing description is considered as illustrative only of the principles of the present disclosure. Further, since numerous modifications and changes will be readily apparent to those skilled in the art, it is not desired to limit the invention to the exact construction and process shown as described above. Accordingly, all suitable modifications and equivalents may be considered to fall within the scope of the invention as defined by the claims that follow.

What is claimed is:

1. A compound of Formula (I) 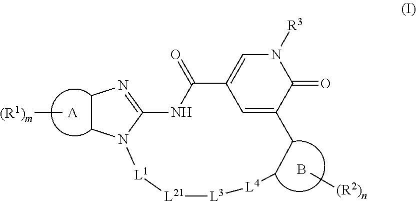
or a pharmaceutically acceptable salt thereof, wherein:
Ring A is selected from the group consisting of cycloalkyl, heterocyclyl, aryl and heteroaryl;
Ring B is selected from the group consisting of cycloalkyl, heterocyclyl, aryl, and heteroaryl;
L¹ is selected from the group consisting of a bond, alkyl, alkenyl, alkynyl, heteroalkyl, heteroalkenyl and heteroalkynyl, wherein the alkyl, alkenyl, alkynyl, heteroalkyl, heteroalkenyl and heteroalkynyl are optionally substituted with one or more groups independently selected from hydroxyl, halogen, cyano and amino;
L² is selected from the group consisting of a bond, N(R^{A}), alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more R^{B};
wherein R^{A} is selected from a group consisting of hydrogen, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, amino, alkyl and haloalkyl;
wherein each R^{B} is independently selected from a group consisting of hydroxyl, halogen, cyano, amino, alkyl, alkenyl, alkynyl, heteroalkyl, heteroalkenyl, cycloalkyl, heterocyclyl, aryl and heteroaryl;
L³ is selected from the group consisting of a bond, alkyl, alkenyl, alkynyl, heteroalkyl, heteroalkenyl and heteroalkynyl, wherein the alkyl, alkenyl, alkynyl, heteroalkyl, heteroalkenyl and heteroalkynyl are optionally substituted with one or more groups independently selected from hydroxyl, halogen, cyano and amino;
L⁴ is selected from O, S, or N(R^{C});
wherein R¹ is selected from a group consisting of hydrogen, alkyl, alkenyl, alkynyl, haloalkyl, heteroalkyl, heteroalkenyl, heteroalkynyl, cycloalkyl, heterocyclyl, aryl, and heteroaryl;
each R¹ is independently selected from the group consisting of hydroxy, halogen, cyano, amino, —N(R^{D})₂, alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more R^{E};
wherein each R^{D} is independently selected from a group consisting of hydrogen, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, —N(R^{F})₂ or —OR^{G};
wherein each R^{E} is independently selected from a group consisting of hydrogen, hydroxyl, halogen, cyano, amino, —N(R^{F})₂, -alkyl-N(R^{F})₂, —C(O)OR^{G}, alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, amino and alkyl;
wherein each of R^{F} and R^{G} is independently selected from a group consisting of alkyl, alkenyl, alkynyl, heteroalkyl, heteroalkenyl, heteroalkynyl, cycloalkyl, heterocyclyl, aryl and heteroaryl;
each R² is independently selected from the group consisting of hydrogen, hydroxy, halogen, cyano, amino, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl and heteroaryl are optionally substituted with one or more groups independently selected from hydroxyl, halogen, cyano, amino, alkyl and haloalkyl;
R³ is selected from the group consisting of hydrogen, alkyl, alkenyl, alkynyl, heteroalkyl, heteroalkenyl, heteroalkynyl, cycloalkyl and heterocyclyl, wherein the alkyl, alkenyl, alkynyl, heteroalkyl, heteroalkenyl, heteroalkynyl, cycloalkyl and heterocyclyl are optionally substituted with one or more groups independently selected from hydroxyl, halogen, cyano, amino, alkyl and haloalkyl;
m is an integer from 0 to 5; and
n is an integer from 0 to 4.

2. The compound of claim 1, or a pharmaceutically acceptable salt thereof, wherein Ring A is aryl or heteroaryl.

3. The compound of claim 2, or a pharmaceutically acceptable salt thereof, wherein Ring A is selected from a group consisting of furanyl, thiophenyl, pyrrolyl, phenyl, pyridinyl, pyranyl, pyrimidinyl, pyridazinyl, pyrazinyl and tetrahydroisoquinoline.

4. The compound of claim 3, or a pharmaceutically acceptable salt thereof, wherein Ring A is selected from a group consisting of

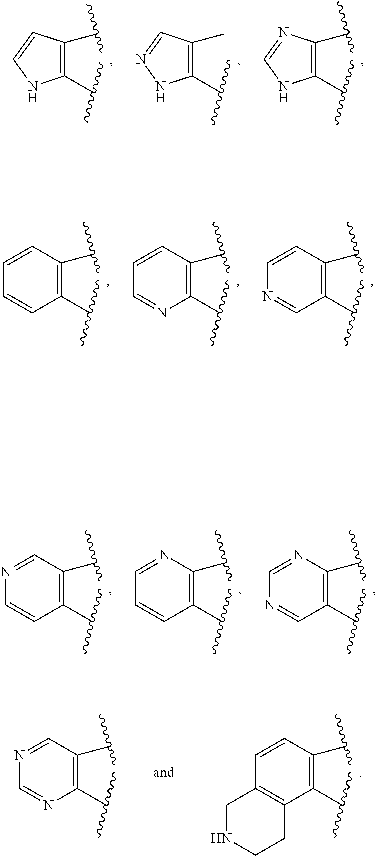

5. The compound of claim 1, or a pharmaceutically acceptable salt thereof, wherein Ring B is aryl or heteroaryl.

6. The compound of claim 5, or a pharmaceutically acceptable salt thereof, wherein Ring B is phenyl, pyridinyl or pyrazolyl.

7. The compound of claim 6, or a pharmaceutically acceptable salt thereof, wherein Ring B is selected from a group consisting of

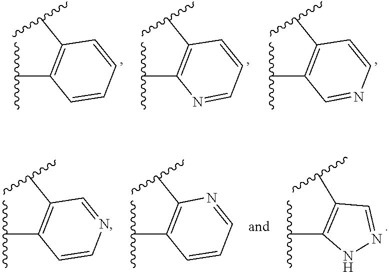

8. The compound of claim 1, or a pharmaceutically acceptable salt thereof, wherein L¹ is a bond.

9. The compound of claim 8, or a pharmaceutically acceptable salt thereof, wherein L¹ is alkyl.

10. The compound of claim 9, or a pharmaceutically acceptable salt thereof, wherein L¹ is selected from a group consisting of 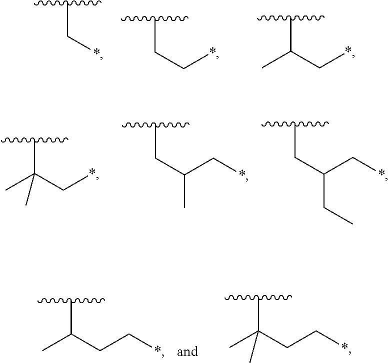
wherein * end of L¹ is connected to L².

11. The compound of claim 1, or a pharmaceutically acceptable salt thereof, wherein L² is a bond.

12. The compound of claim 1, or a pharmaceutically acceptable salt thereof, wherein L² is N(R^{A}), and R^{A} is selected from alkyl or heterocyclyl, wherein the alkyl or heterocyclyl are are optionally substituted with one or more halogen or alkyl.

13. The compound of claim 12, or a pharmaceutically acceptable salt thereof, wherein R^{A} is ethyl, difluoroethyl, trifluoroethyl or oxetanyl.

14. The compound of claim 1, or a pharmaceutically acceptable salt thereof, wherein L² is cycloalkyl optionally substituted with one or more R^{B}.

15. The compound of claim 14, or a pharmaceutically acceptable salt thereof, wherein L² is 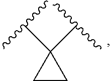 [SMILES: *C1(*)CC1]
which is optionally substituted with one or more R^{B}.

16. The compound of claim 1, or a pharmaceutically acceptable salt thereof, wherein L² is heterocyclyl optionally substituted with one or more R^{B}.

17. The compound of claim 16, or a pharmaceutically acceptable salt thereof, wherein the heterocyclyl is selected from a group consisting of 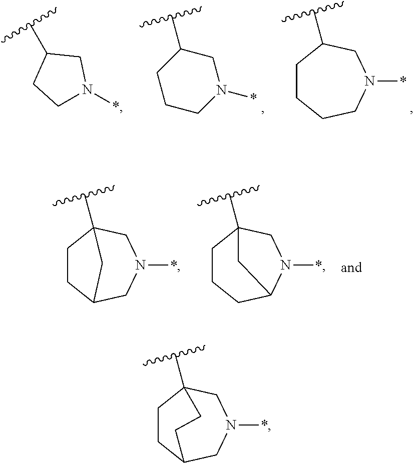
each of which is optionally substituted with one or more R^{B}, wherein * end of L² is connected to L³.

18. The compound of claim 16 or 17, or a pharmaceutically acceptable salt thereof, wherein R^{B} is alkyl.

19. The compound of claim 19, or a pharmaceutically acceptable salt thereof, wherein R^{B} is methyl.

20. The compound of claim 1, or a pharmaceutically acceptable salt thereof, wherein L² is selected from a group consisting of: 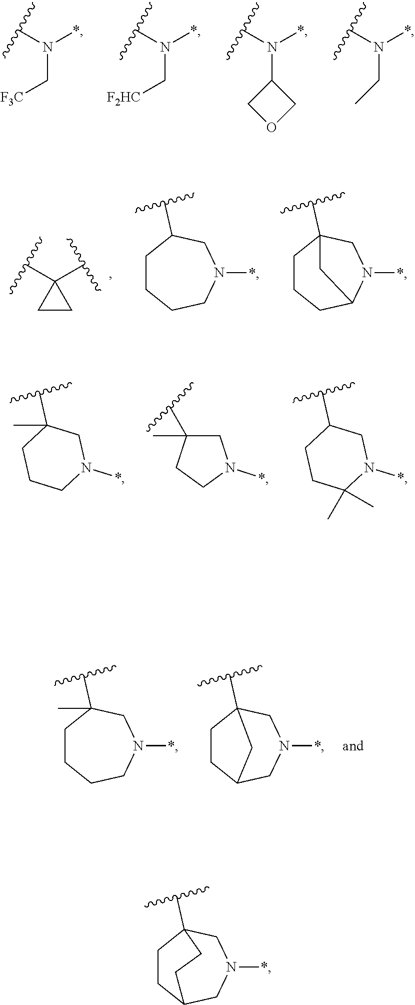
wherein * end of L² is connected to L³.

21. The compound of claim 1, or a pharmaceutically acceptable salt thereof, wherein L³ is alkyl.

22. The compound of claim 21, or a pharmaceutically acceptable salt thereof, wherein L³ is ethyl.

23. The compound of claim 1, or a pharmaceutically acceptable salt thereof, wherein L⁴ is O or NH.

24. The compound of claim 1, or a pharmaceutically acceptable salt thereof, wherein L¹ is a bond and L² is a bond or heterocyclyl optionally substituted with one or more R^{B}.

25. The compound of claim 1, or a pharmaceutically acceptable salt thereof, wherein L¹ is alkyl and L² is a bond, N(R^{A}), or cycloalkyl optionally substituted with one or more R^{B}.

26. The compound of claim 1, or a pharmaceutically acceptable salt thereof, wherein R¹ is hydroxy and m is 1.

27. The compound of claim 1, or a pharmaceutically acceptable salt thereof, wherein R¹ is halogen and m is 1.

28. The compound of claim 27, or a pharmaceutically acceptable salt thereof, wherein R, is bromo or fluoro.

29. The compound of claim 1, or a pharmaceutically acceptable salt thereof, wherein R¹ is —N(R^{D})₂ and m is 1.

30. The compound of claim 29, or a pharmaceutically acceptable salt thereof, wherein each R^{D} is independently hydrogen, or alkyl optionally substituted with one or more groups independently selected from —N(R^{F})₂ or —OR^{G}.

31. The compound of claim 30, or a pharmaceutically acceptable salt thereof, wherein each R^{D} is independently hydrogen, methyl, methoxyethyl, N,N-dimethylaminoethyl, hydroxyethyl or N,N-dimethylaminopropyl.

32. The compound of claim 1, or a pharmaceutically acceptable salt thereof, wherein R¹ is heterocyclyl optionally substituted with one or more R^{E} and m is 1.

33. The compound of claim 32, or a pharmaceutically acceptable salt thereof, wherein the heterocyclyl is selected from a group consisting of 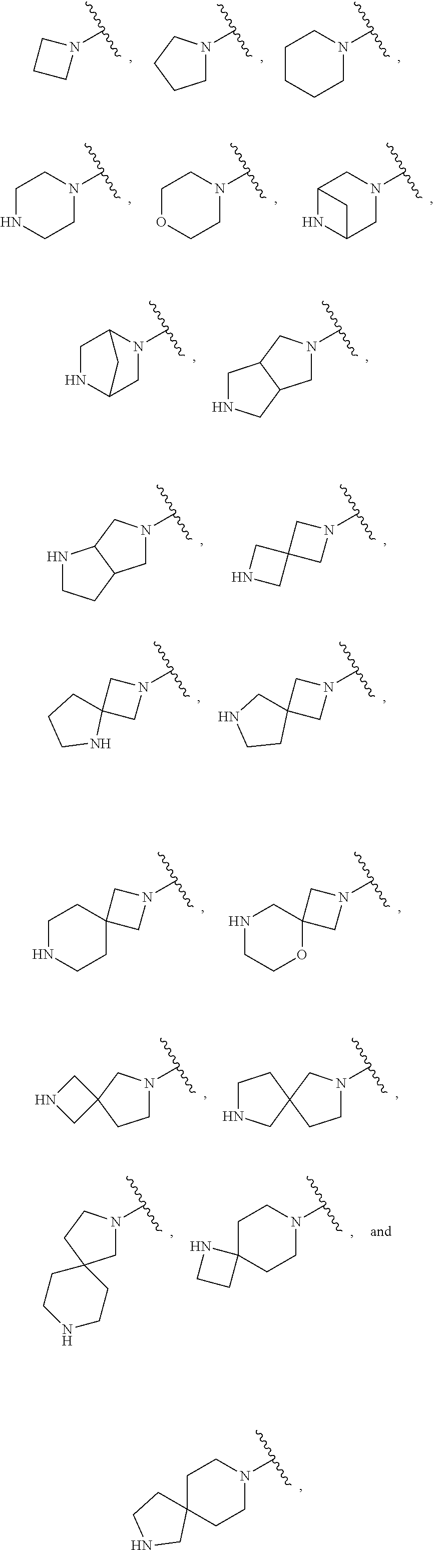
each of which is optionally substituted with one or more R^{E}.

34. The compound of claim 32, or a pharmaceutically acceptable salt thereof, wherein R^{E} is halogen.

35. The compound of claim 33, or a pharmaceutically acceptable salt thereof, wherein R^{E} is F.

36. The compound of claim 32, or a pharmaceutically acceptable salt thereof, wherein each R^{E} is independently selected from halogen, —N(R^{F})₂, -alkyl-N(R^{F})₂, —C(O)OR^{G}, or alkyl optionally substituted with one or more halogen.

37. The compound of claim 36, or a pharmaceutically acceptable salt thereof, wherein each of R^{F} and R^{G} is independently alkyl.

38. The compound of claim 36, or a pharmaceutically acceptable salt thereof, wherein each of R^{F} and R^{G} is independently C₁₋₃ alkyl.

39. The compound of claim 36, or a pharmaceutically acceptable salt thereof, wherein each R^{E} is independently selected from a group consisting of F, —N(CH₃)₂,—C₁₋₃ alkyl-N(CH₃)₂, —C(O)O(tert-butyl), methyl, ethyl, or trifluoroethyl.

40. The compound of claim 32, or a pharmaceutically acceptable salt thereof, wherein R^{E} is cycloalkyl or heterocyclyl, wherein the cycloalkyl and heterocyclyl are optionally substituted with one or more alkyl or halogen.

41. The compound of claim 40, or a pharmaceutically acceptable salt thereof, wherein R^{E} is selected from cyclopropyl, morpholinyl, piperazinyl, oxetyl or azetidinyl, each of which is optionally substituted with one or more alkyl or halogen.

42. The compound of claim 1, or a pharmaceutically acceptable salt thereof, wherein m is 2, one of R¹ is halogen, and the other R¹ is heterocyclyl optionally substituted with one or more R^{E}.

43. The compound of claim 42, or a pharmaceutically acceptable salt thereof, wherein the heterocyclyl is selected from a group consisting of 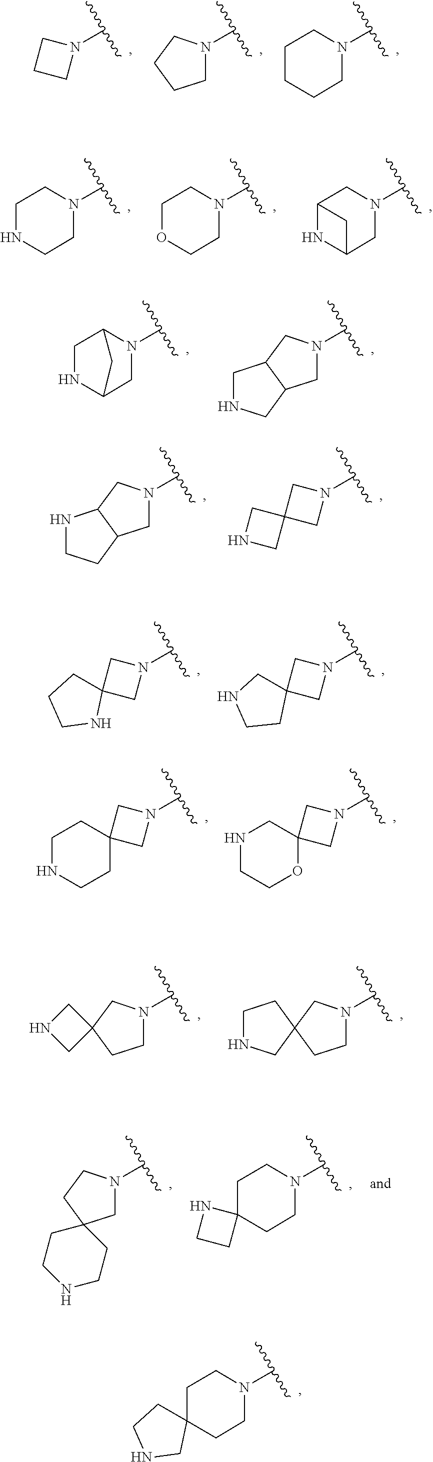
each of which is optionally substituted with one or more R^{E}.

44. The compound of claim 43, or a pharmaceutically acceptable salt thereof, wherein each R^{E} is independently selected from halogen, —N(R^{F})₂, -alkyl-N(R^{F})₂, —C(O)OR^{G}, or alkyl optionally substituted with one or more halogen.

45. The compound of claim 1, or a pharmaceutically acceptable salt thereof, wherein R¹ is selected from the group consisting of

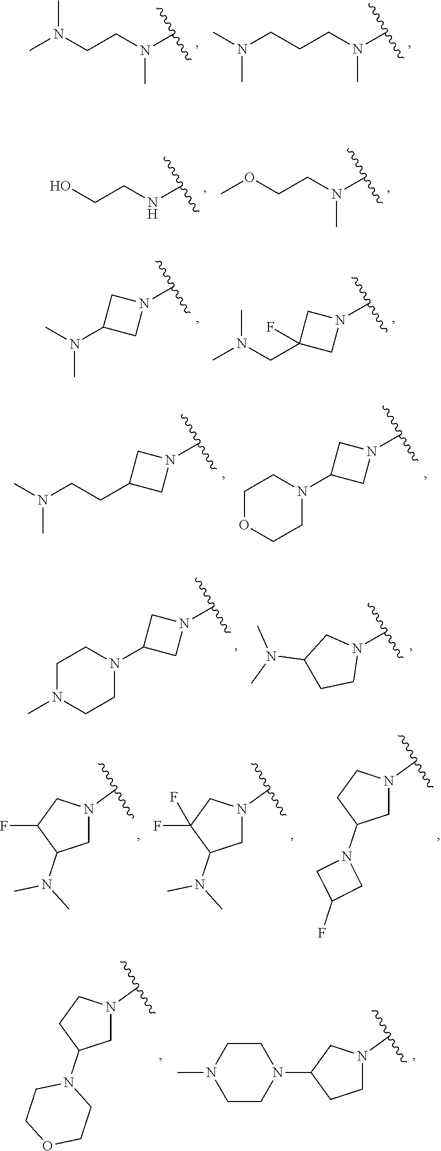

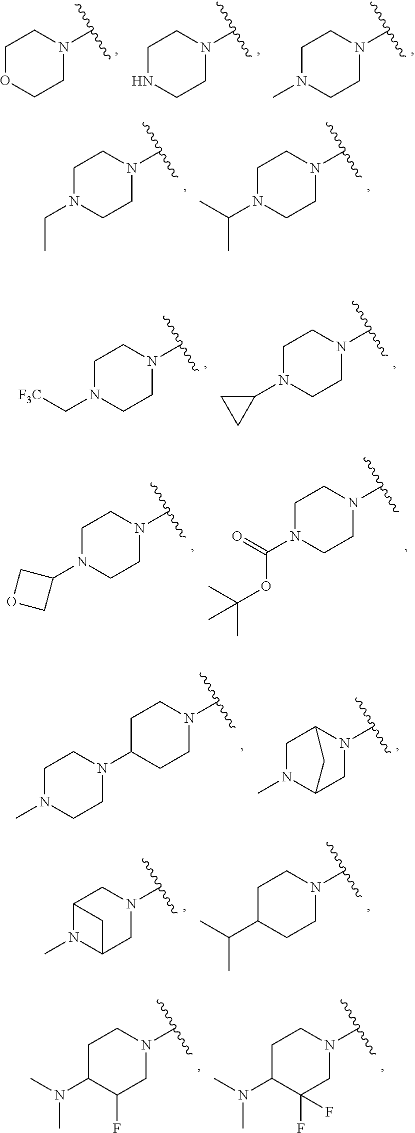

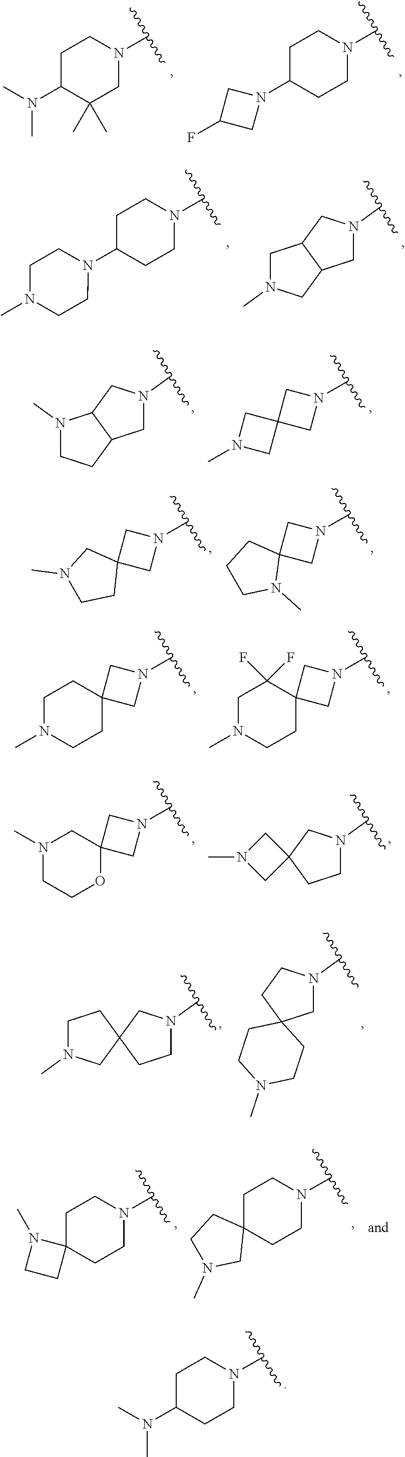

46. The compound of claim 1, or a pharmaceutically acceptable salt thereof, wherein R² is halogen and n is 1 or 2.

47. The compound of claim 46, or a pharmaceutically acceptable salt thereof, wherein R² is F.

48. The compound of claim 1, or a pharmaceutically acceptable salt thereof, wherein R² is alkyl or cycloalkyl, wherein the alkyl and cycloalkyl are optionally substituted with one or more halogen.

49. The compound of claim 48, or a pharmaceutically acceptable salt thereof, wherein R² is methyl, ethyl, cyclopropyl or trifluoroethyl.

50. The compound of claim 1, or a pharmaceutically acceptable salt thereof, wherein R³ is alkyl or cycloalkyl.

51. The compound of claim 50, or a pharmaceutically acceptable salt thereof, wherein R³ is methyl, ethyl or cyclopropyl.

52. The compound of claim 1, or a pharmaceutically acceptable salt thereof, wherein m is 0, 1 or 2.

53. The compound of claim 1, or a pharmaceutically acceptable salt thereof, wherein n is 0 or 1.

54. The compound of claim 1, or a pharmaceutically acceptable salt thereof, wherein the compound is selected from the group consisting of:

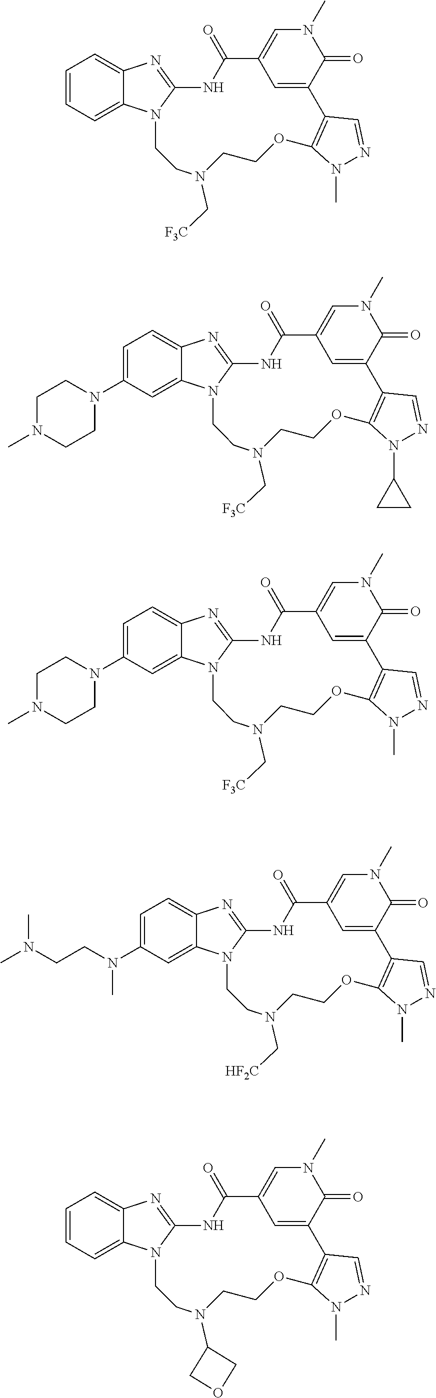

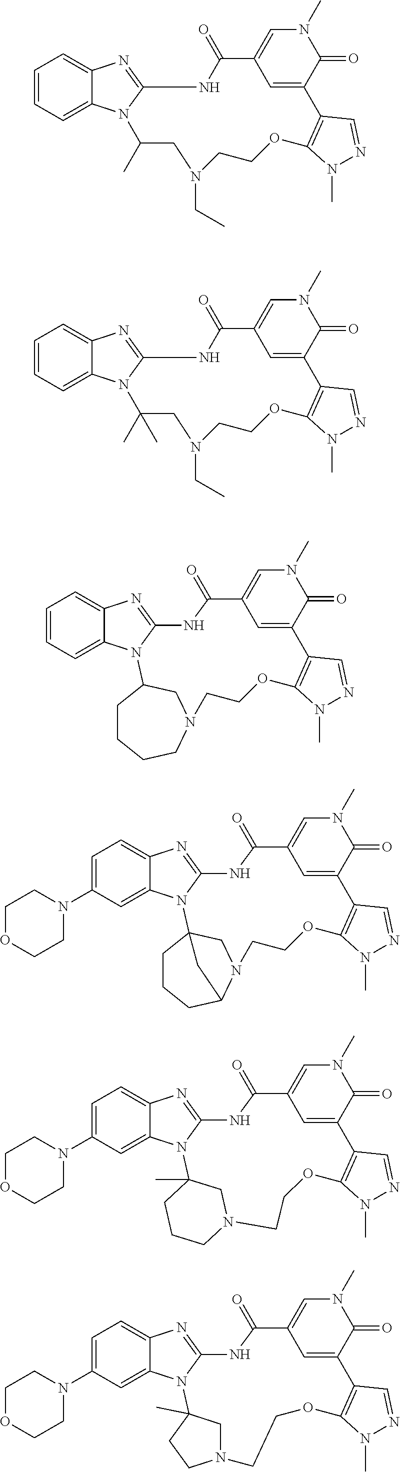

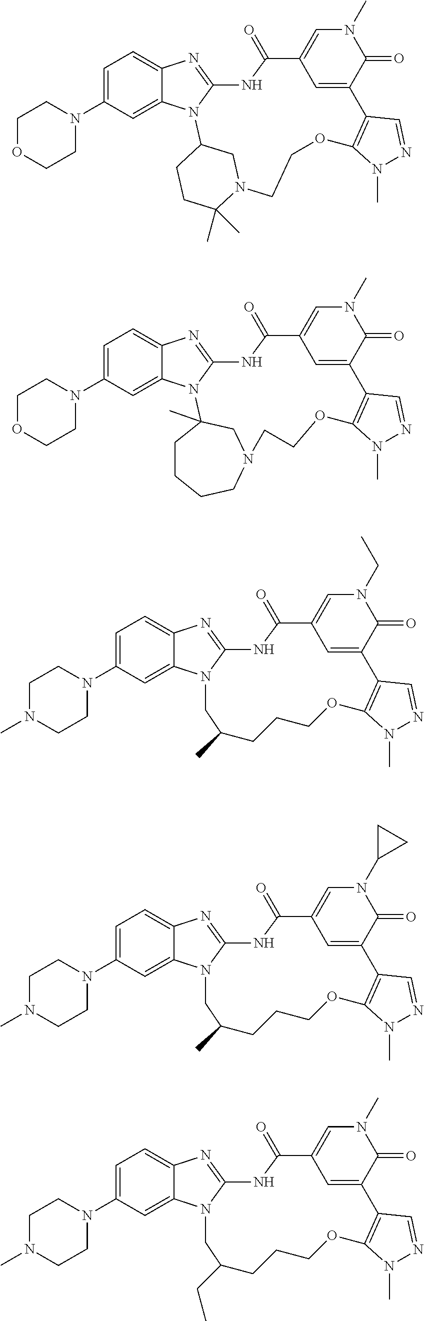

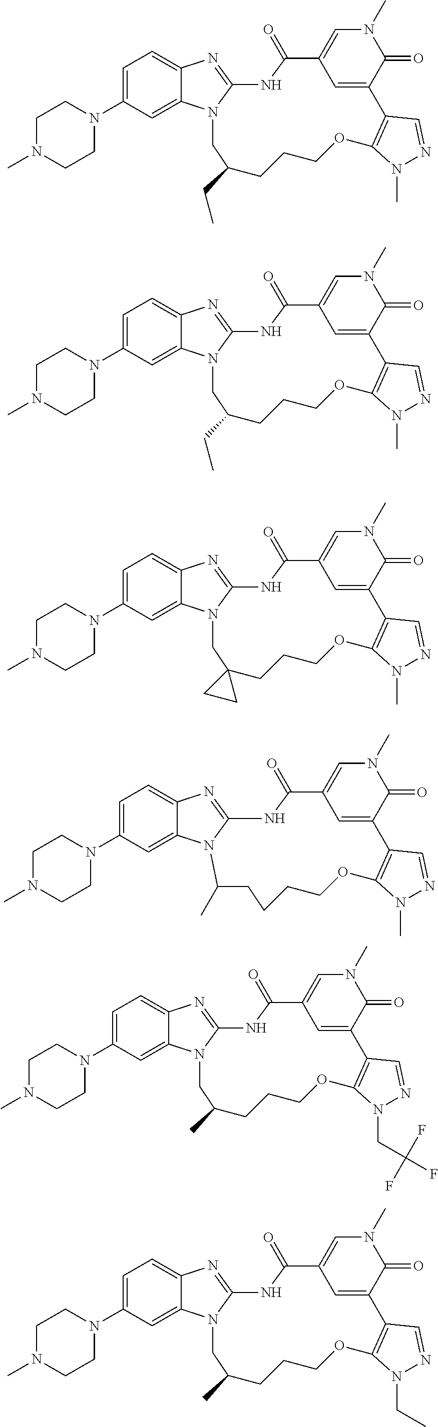

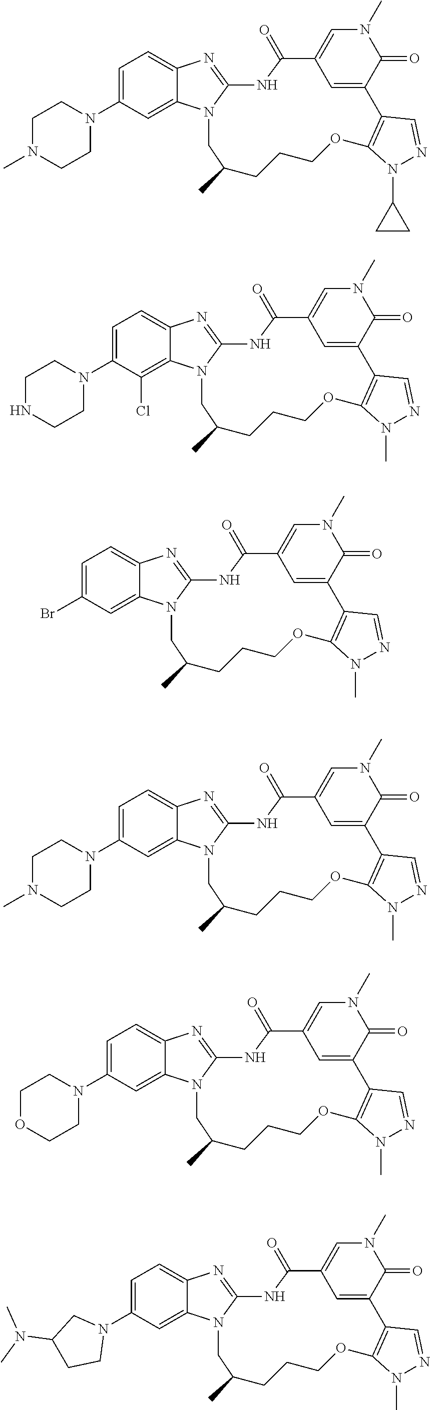

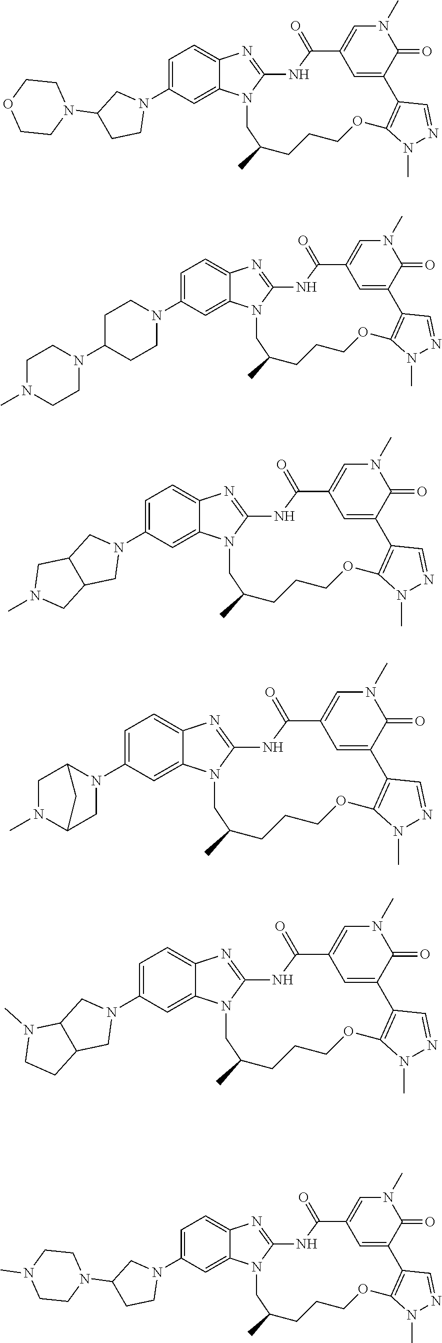

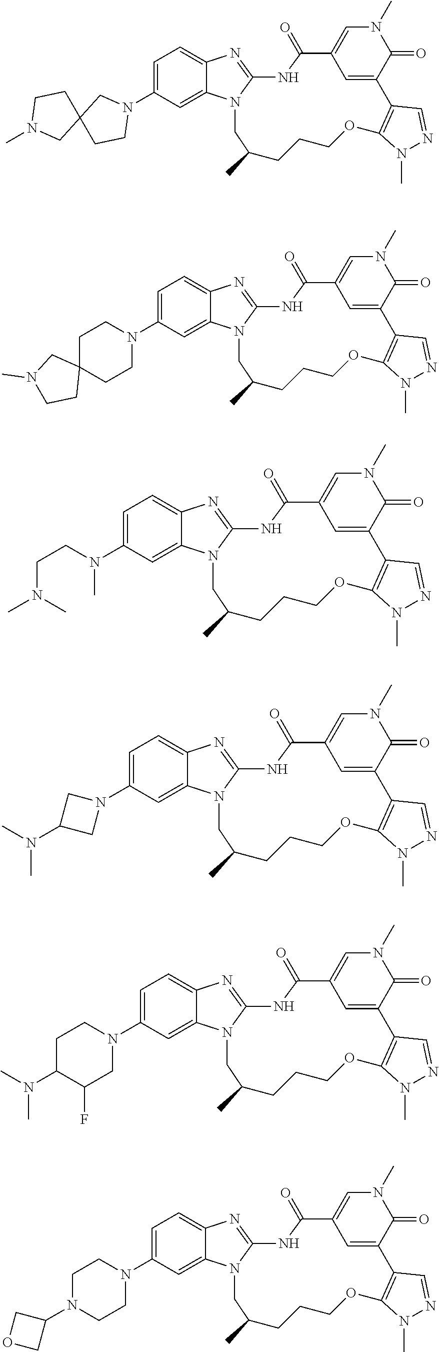

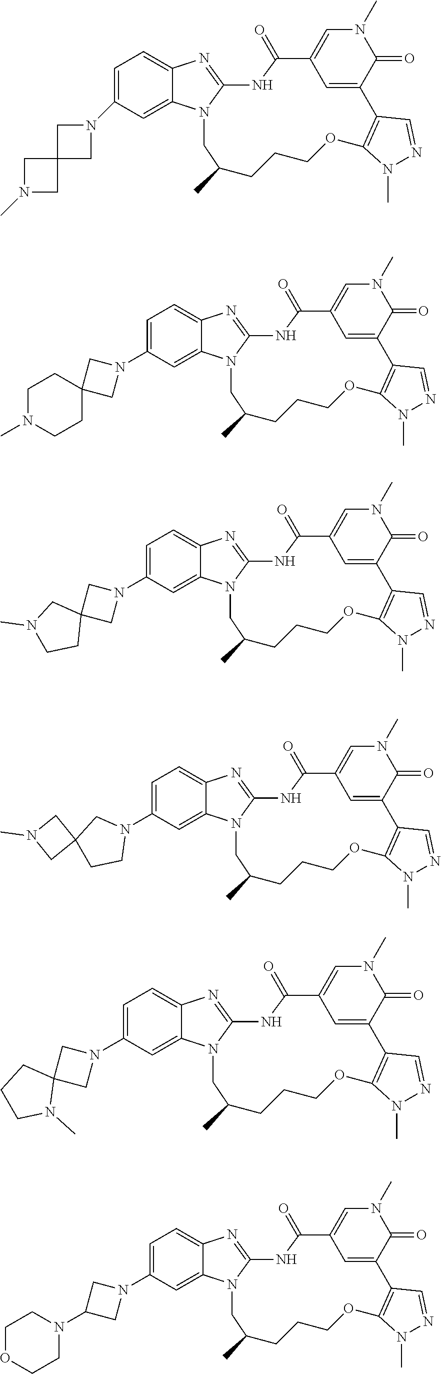

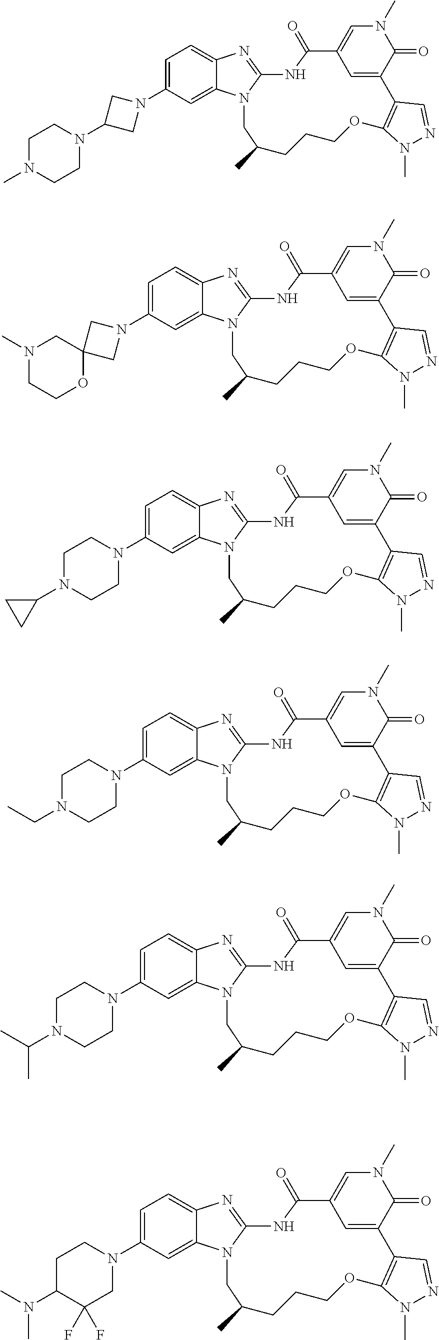

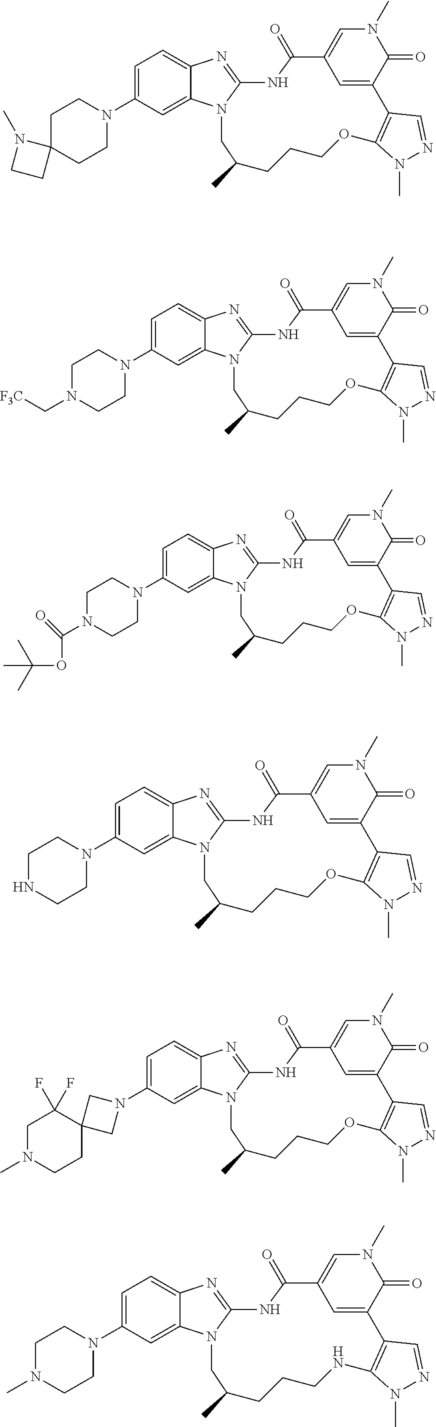 [SMILES: C[C@@H]1CCCOc2c(cnn2C)-c2cc(cn(C)c2=O)C(=O)Nc2nc3ccc(N4CCC5(CC4)CCN5C)cc3n2C1 ; C[C@@H]1CCCOc2c(cnn2C)-c2cc(cn(C)c2=O)C(=O)Nc2nc3ccc(N4CCN(CC(F)(F)F)CC4)cc3n2C1 ; C[C@@H]1CCCOc2c(cnn2C)-c2cc(cn(C)c2=O)C(=O)Nc2nc3ccc(N4CCN(C(=O)OC(C)(C)C)CC4)cc3n2C1 ; C[C@@H]1CCCOc2c(cnn2C)-c2cc(cn(C)c2=O)C(=O)Nc2nc3ccc(N4CCNCC4)cc3n2C1 ; C[C@@H]1CCCOc2c(cnn2C)-c2cc(cn(C)c2=O)C(=O)Nc2nc3ccc(N4CC5(CCN(C)CC5(F)F)C4)cc3n2C1 ; C[C@@H]1CCCNc2c(cnn2C)-c2cc(cn(C)c2=O)C(=O)Nc2nc3ccc(N4CCN(C)CC4)cc3n2C1]

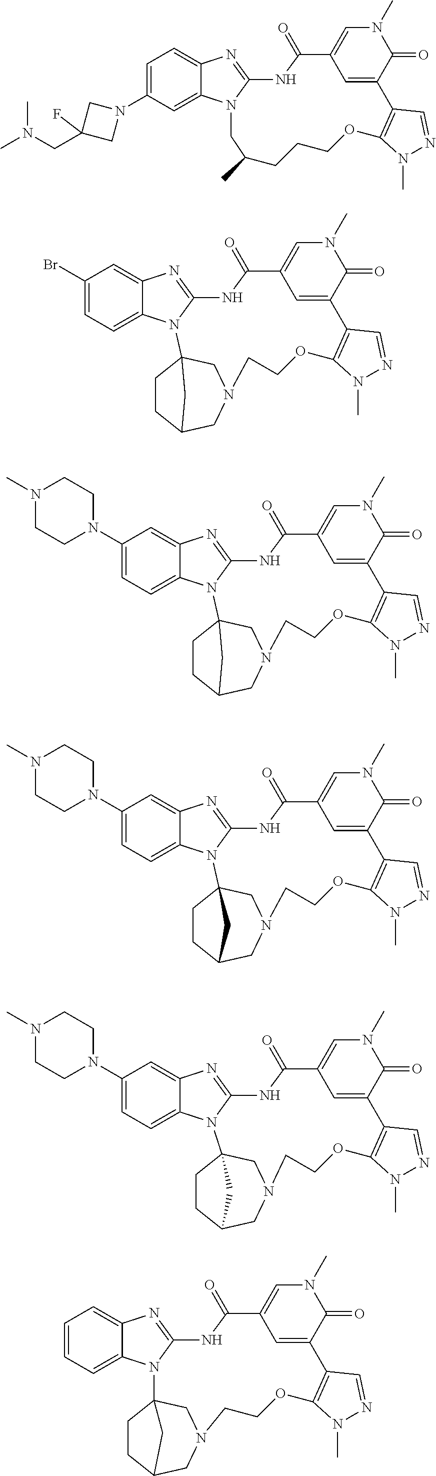 [SMILES: C[C@@H]1CCCOc2c(cnn2C)-c2cc(cn(C)c2=O)C(=O)Nc2nc3ccc(N4CC(F)(CN(C)C)C4)cc3n2C1 ; Cn1ncc2c1OCCN1CC3CCC(C3)(C1)n1c(nc3cc(Br)ccc31)NC(=O)c1cc-2c(=O)n(C)c1 ; CN1CCN(c2ccc3c(c2)nc2n3C34CCC(CN(CCOc5c(cnn5C)-c5cc(cn(C)c5=O)C(=O)N2)C3)C4)CC1 ; CN1CCN(c2ccc3c(c2)nc2n3[C@@]34CC[C@@H](CN(CCOc5c(cnn5C)-c5cc(cn(C)c5=O)C(=O)N2)C3)C4)CC1 ; CN1CCN(c2ccc3c(c2)nc2n3[C@]34CC[C@H](CN(CCOc5c(cnn5C)-c5cc(cn(C)c5=O)C(=O)N2)C3)C4)CC1 ; Cn1ncc2c1OCCN1CC3CCC(C3)(C1)n1c(nc3ccccc31)NC(=O)c1cc-2c(=O)n(C)c1]

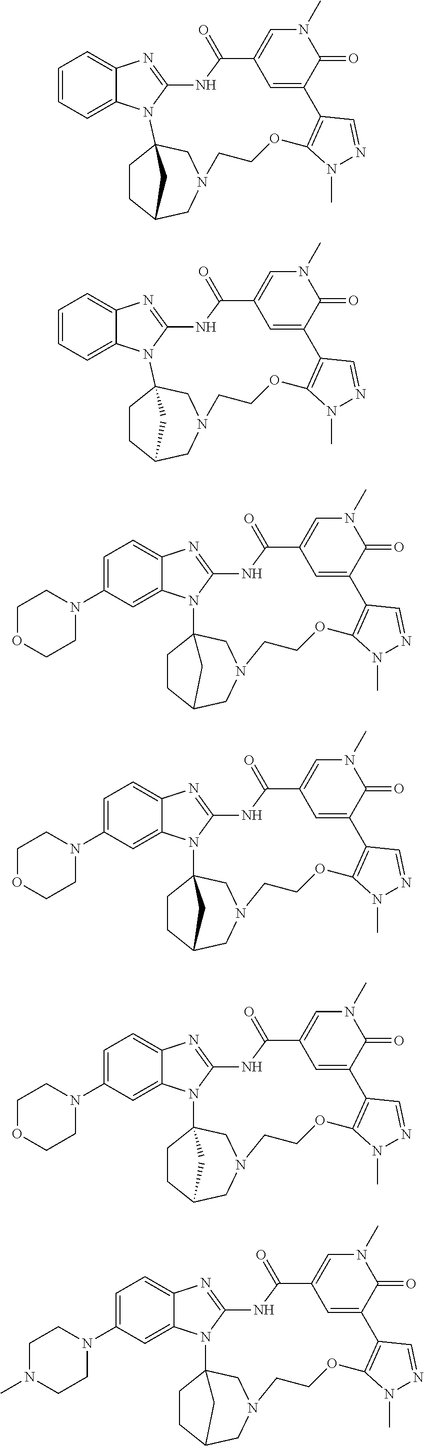

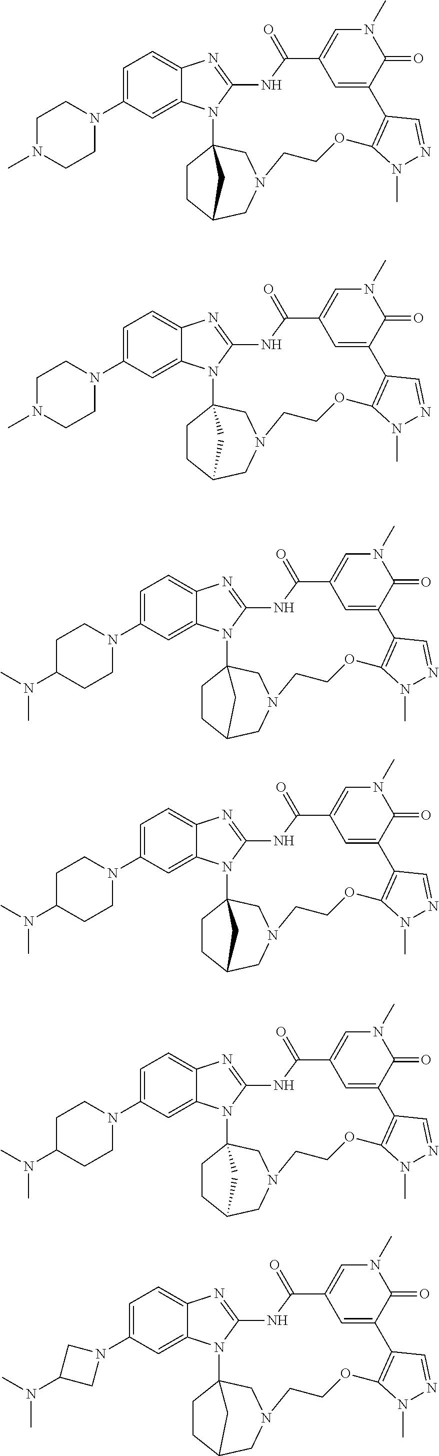

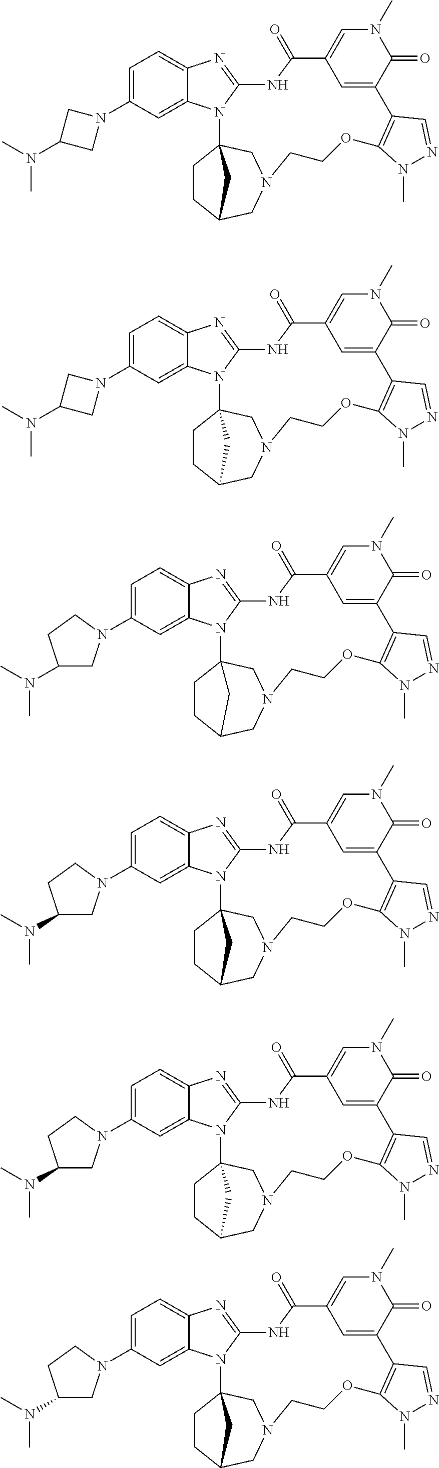

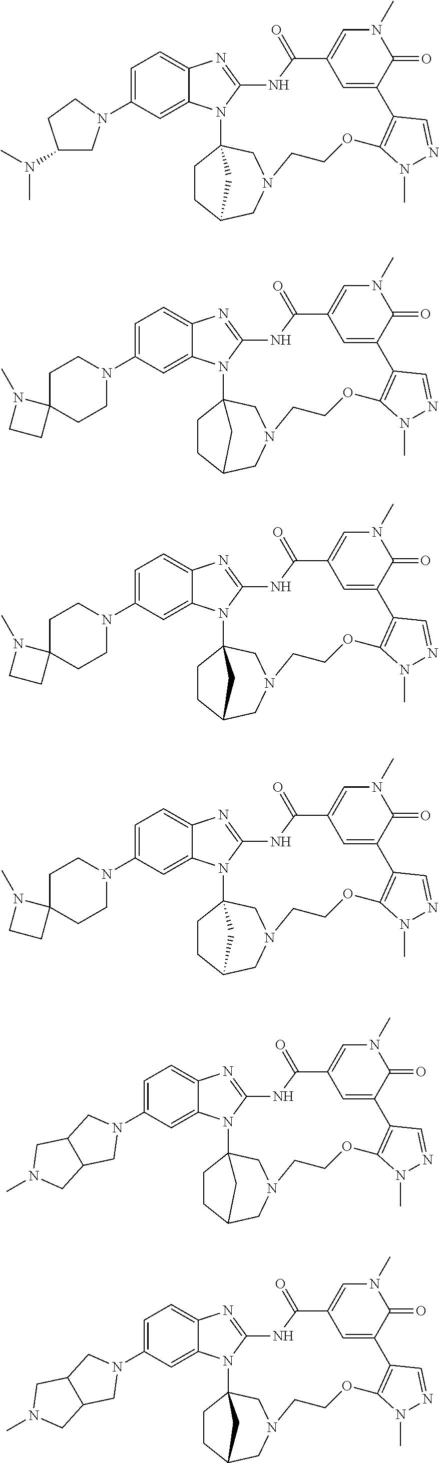

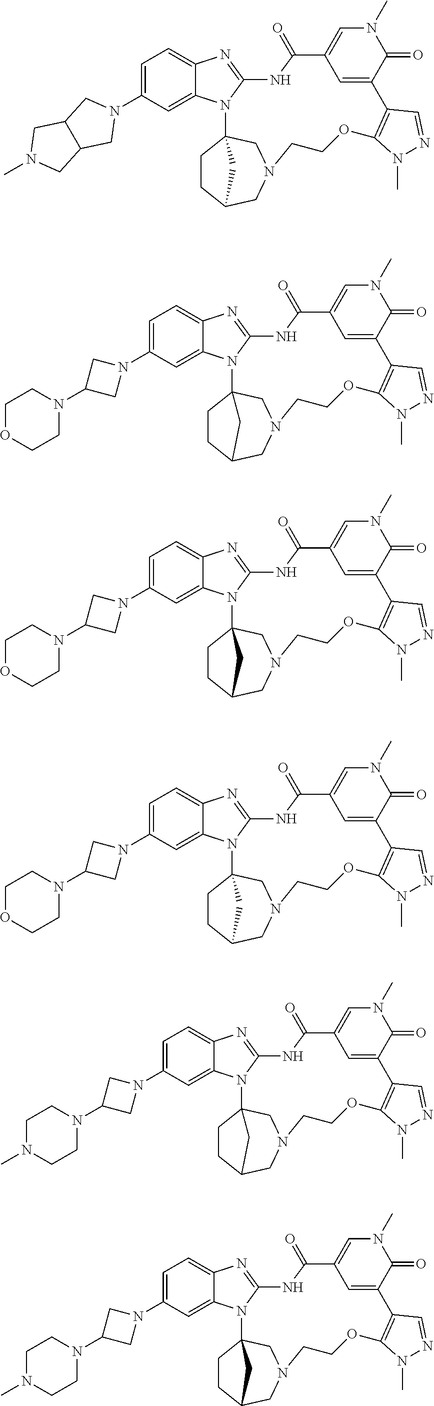

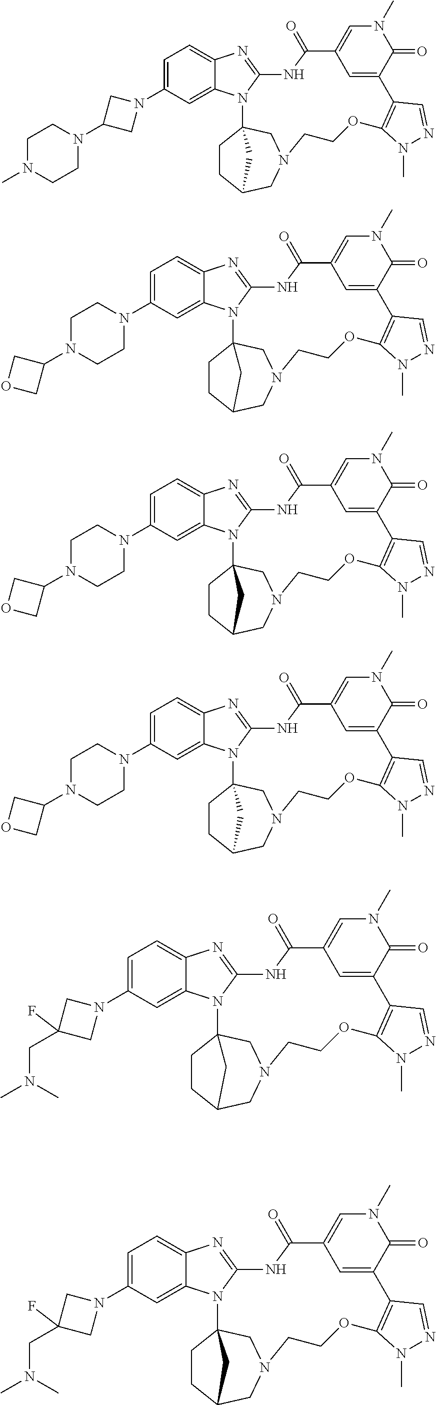

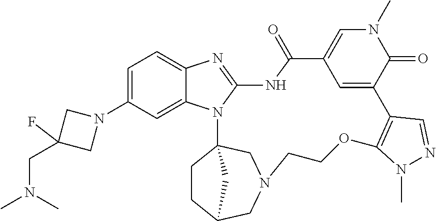 [SMILES: CN(C)CC1(F)CN(c2ccc3nc4n(c3c2)[C@]23CC[C@H](CN(CCOc5c(cnn5C)-c5cc(cn(C)c5=O)C(=O)N4)C2)C3)C1]

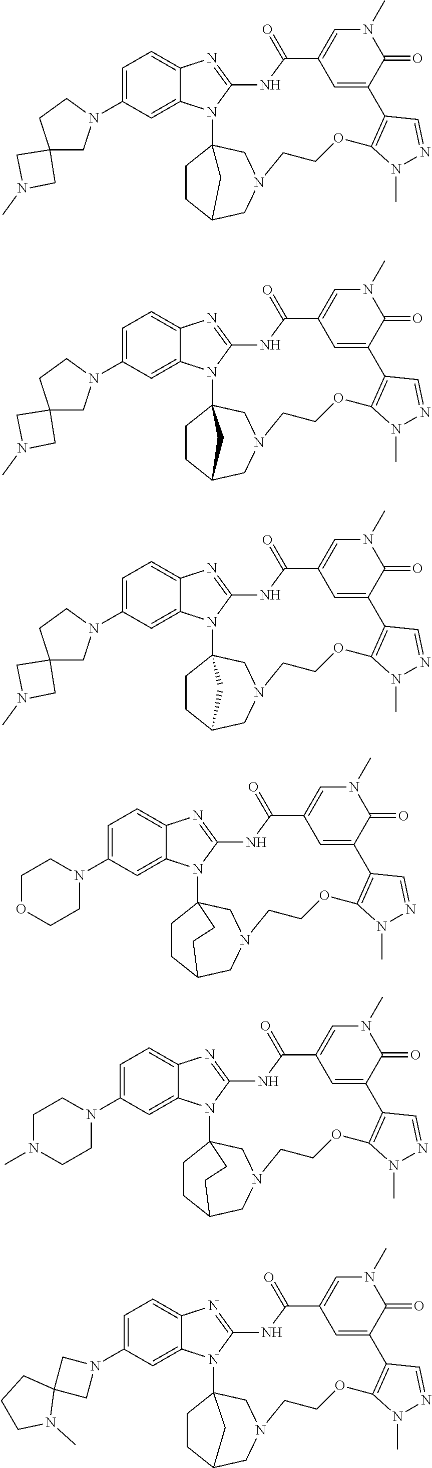

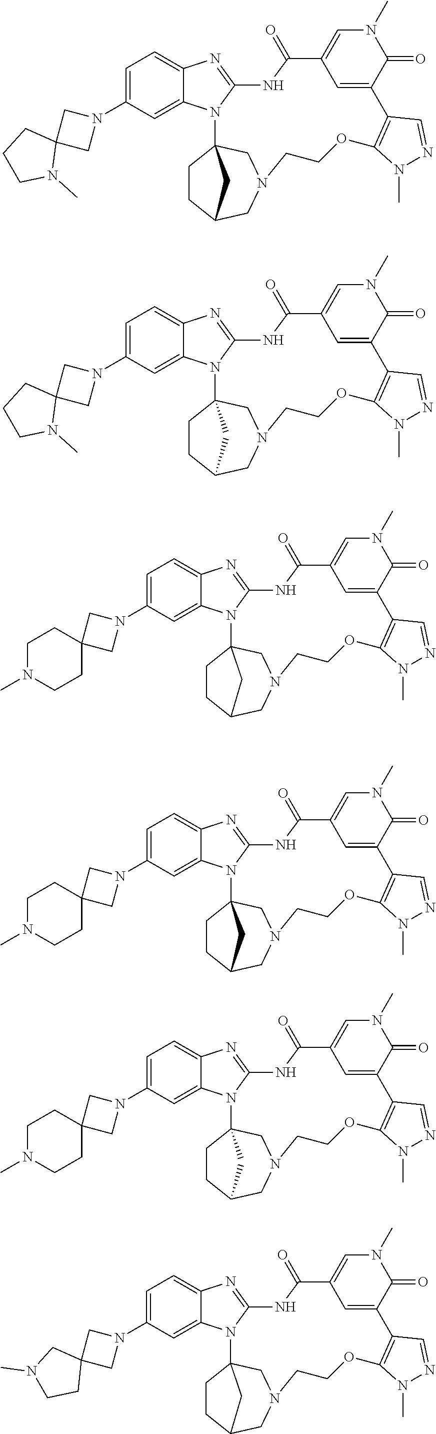

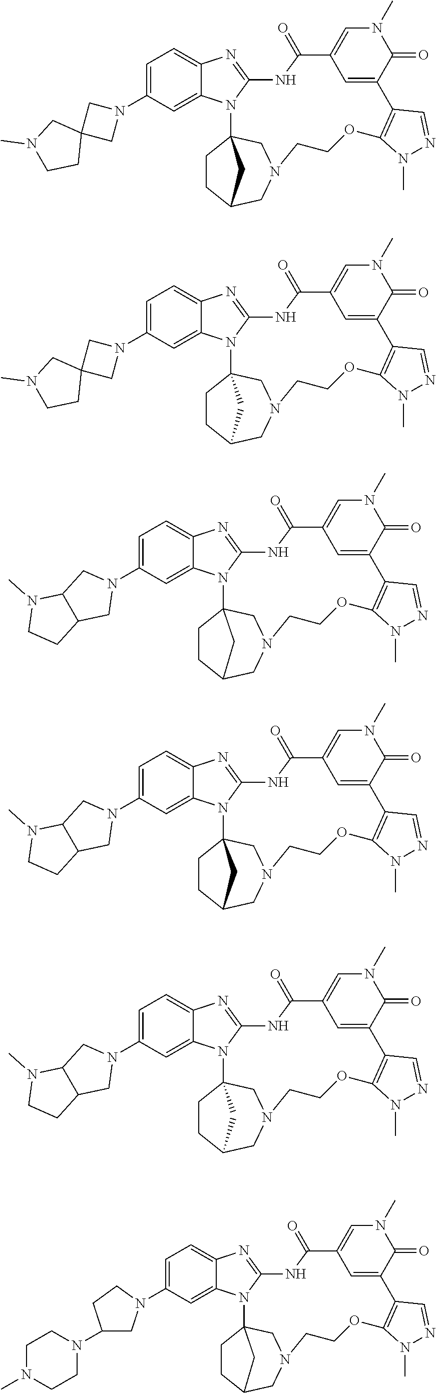

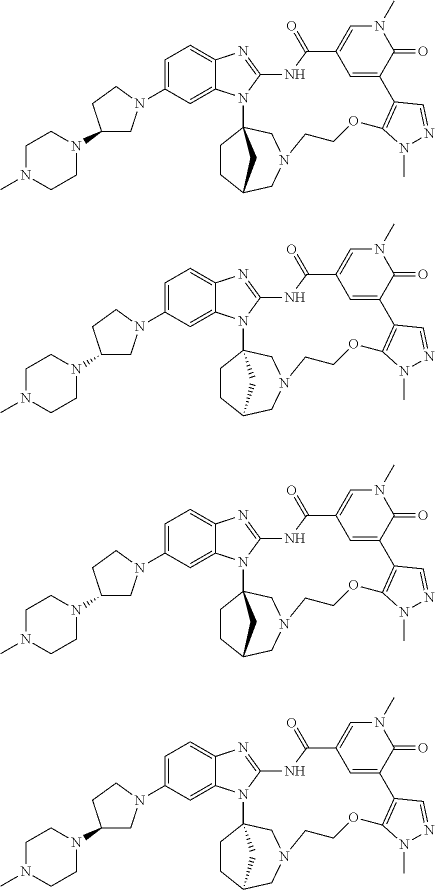 [SMILES: CN1CCN([C@H]2CCN(c3ccc4nc5n(c4c3)[C@@]34CC[C@@H](CN(CCOc6c(cnn6C)-c6cc(cn(C)c6=O)C(=O)N5)C3)C4)C2)CC1 ; CN1CCN([C@@H]2CCN(c3ccc4nc5n(c4c3)[C@]34CC[C@H](CN(CCOc6c(cnn6C)-c6cc(cn(C)c6=O)C(=O)N5)C3)C4)C2)CC1 ; CN1CCN([C@@H]2CCN(c3ccc4nc5n(c4c3)[C@@]34CC[C@@H](CN(CCOc6c(cnn6C)-c6cc(cn(C)c6=O)C(=O)N5)C3)C4)C2)CC1 ; CN1CCN([C@H]2CCN(c3ccc4nc5n(c4c3)[C@]34CC[C@H](CN(CCOc6c(cnn6C)-c6cc(cn(C)c6=O)C(=O)N5)C3)C4)C2)CC1]

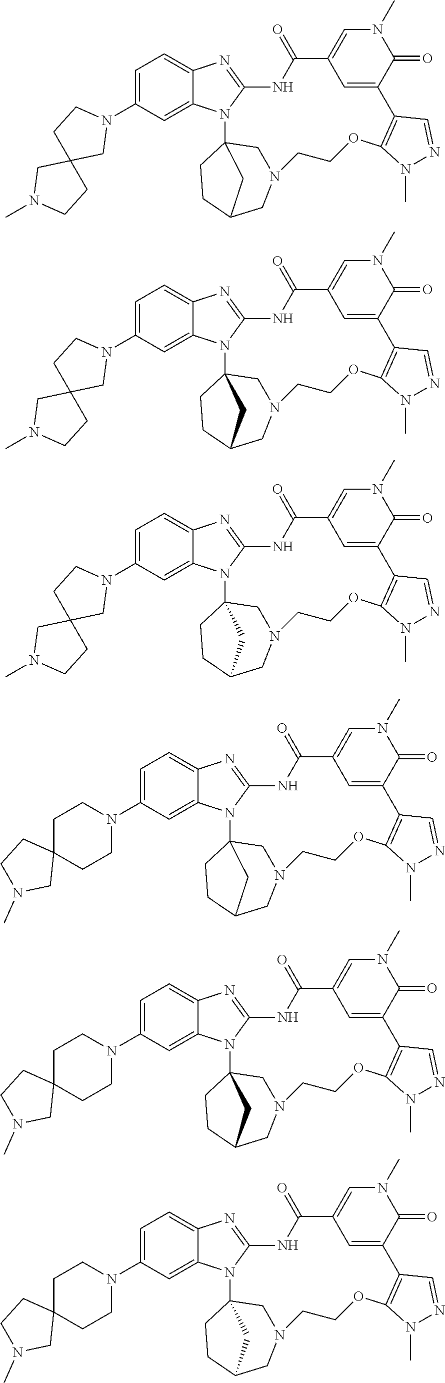

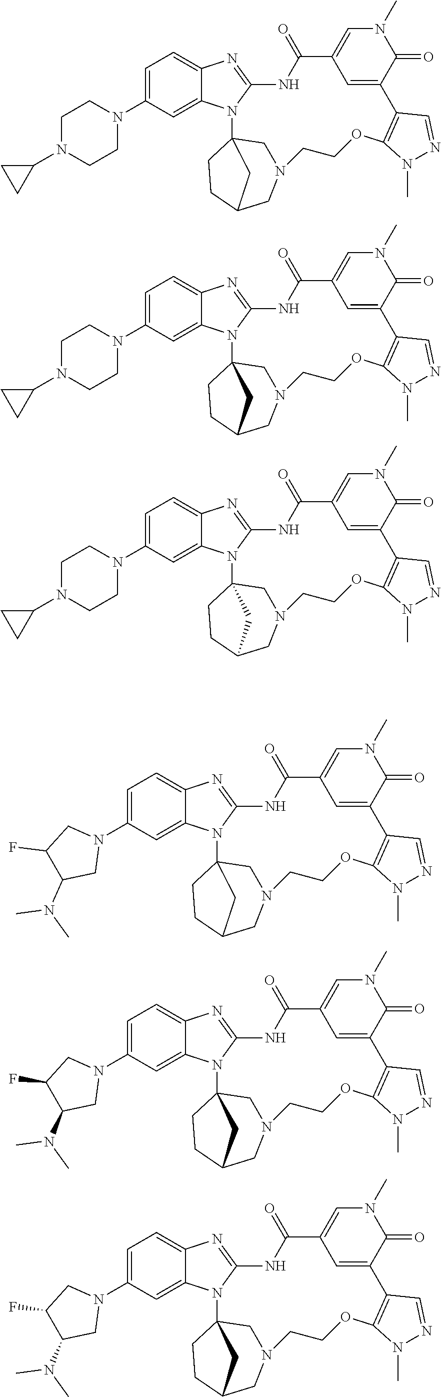

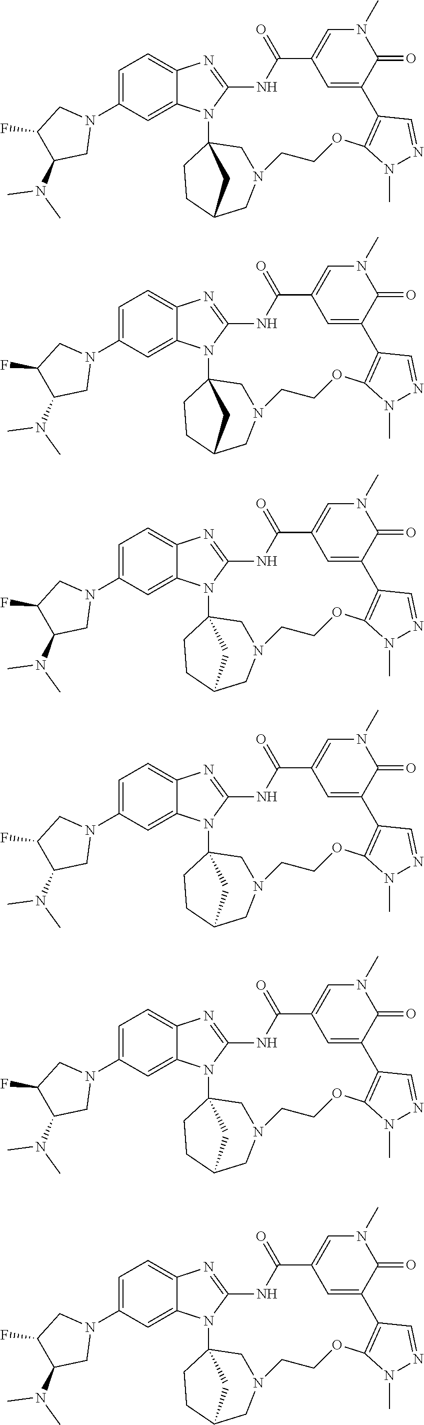

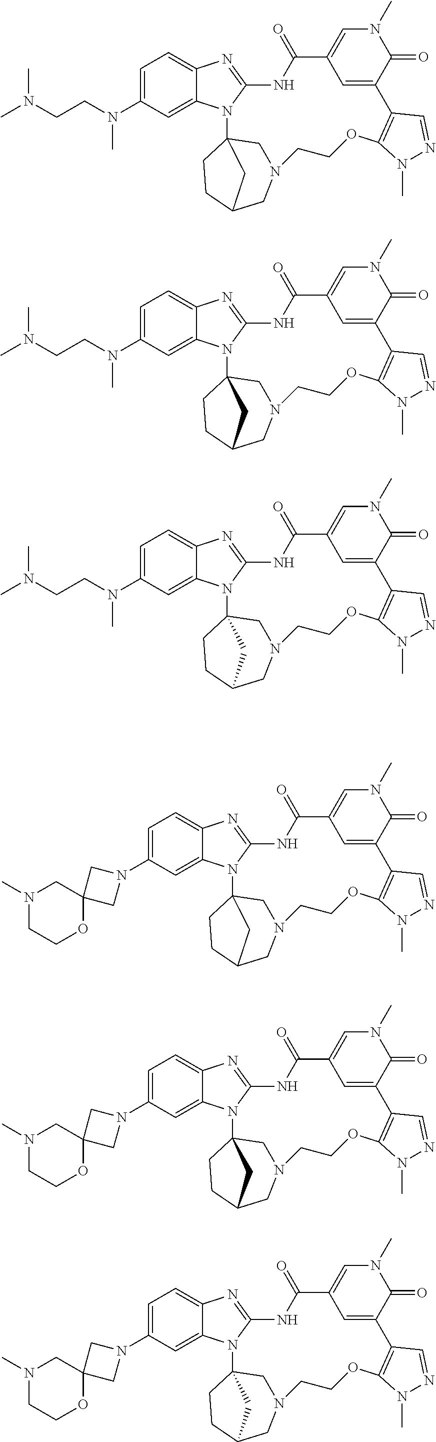

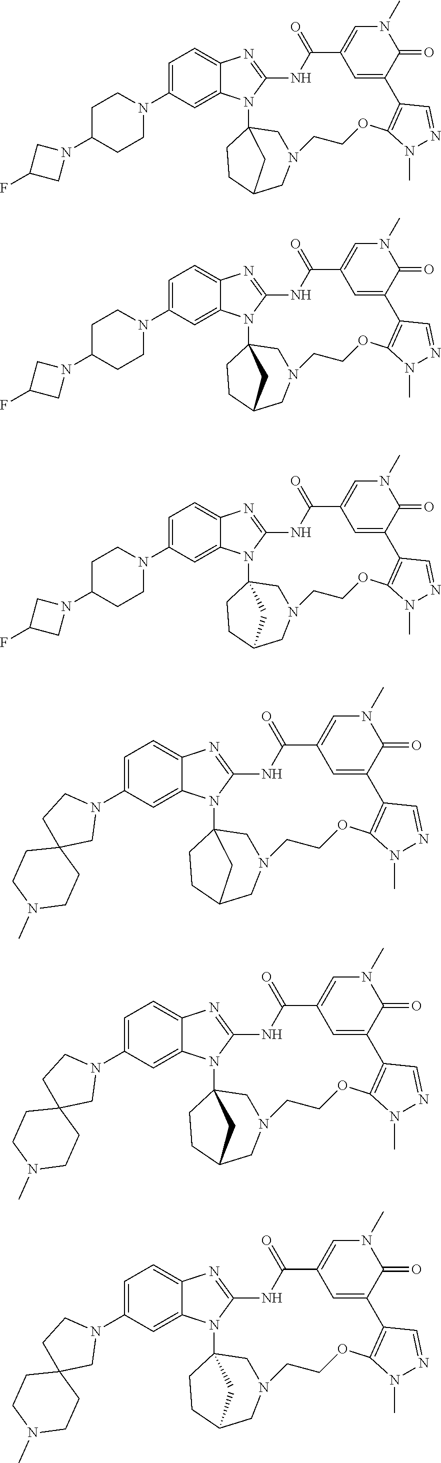

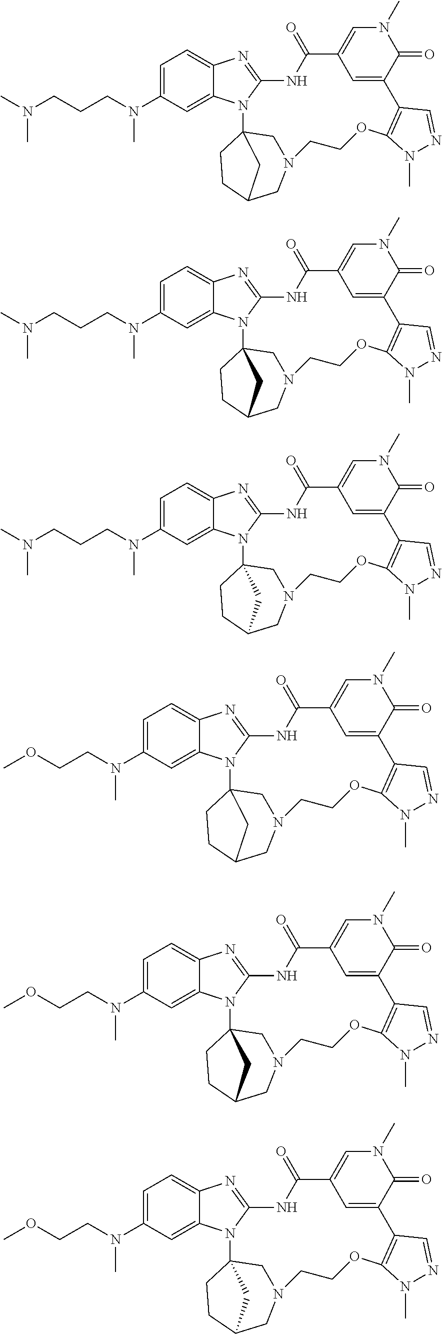

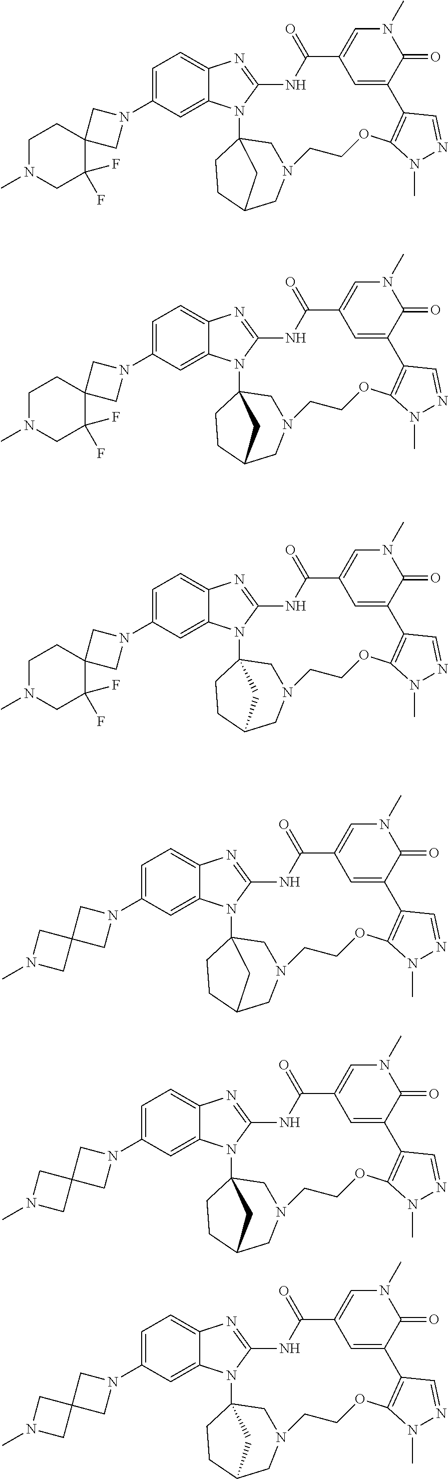

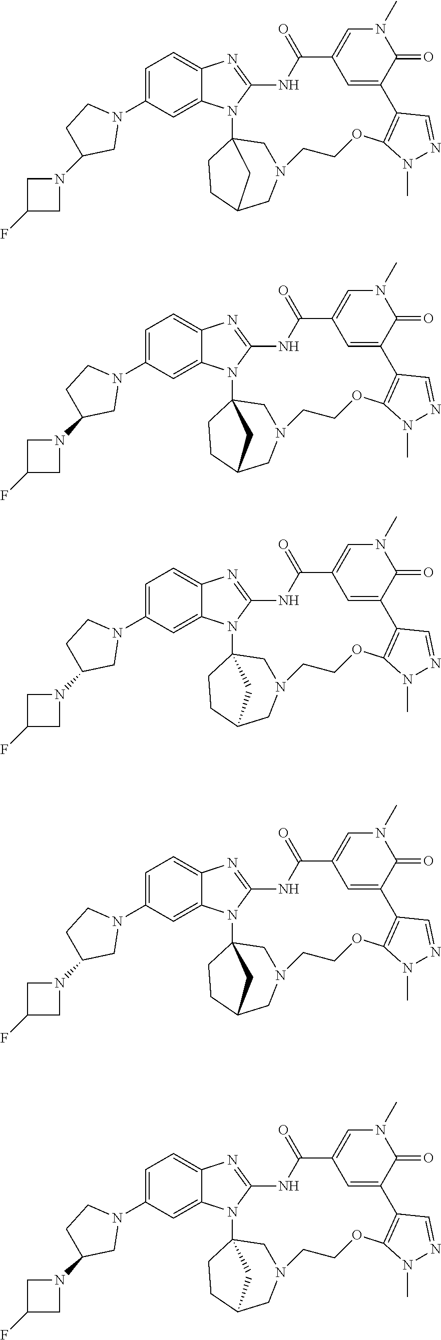 [SMILES: Cn1ncc2c1OCCN1CC3CCC(C3)(C1)n1c(nc3ccc(N4CCC(N5CC(F)C5)C4)cc31)NC(=O)c1cc-2c(=O)n(C)c1 ; Cn1ncc2c1OCCN1C[C@@H]3CC[C@@](C3)(C1)n1c(nc3ccc(N4CC[C@H](N5CC(F)C5)C4)cc31)NC(=O)c1cc-2c(=O)n(C)c1 ; Cn1ncc2c1OCCN1C[C@H]3CC[C@](C3)(C1)n1c(nc3ccc(N4CC[C@@H](N5CC(F)C5)C4)cc31)NC(=O)c1cc-2c(=O)n(C)c1 ; Cn1ncc2c1OCCN1C[C@@H]3CC[C@@](C3)(C1)n1c(nc3ccc(N4CC[C@@H](N5CC(F)C5)C4)cc31)NC(=O)c1cc-2c(=O)n(C)c1 ; Cn1ncc2c1OCCN1C[C@H]3CC[C@](C3)(C1)n1c(nc3ccc(N4CC[C@H](N5CC(F)C5)C4)cc31)NC(=O)c1cc-2c(=O)n(C)c1]

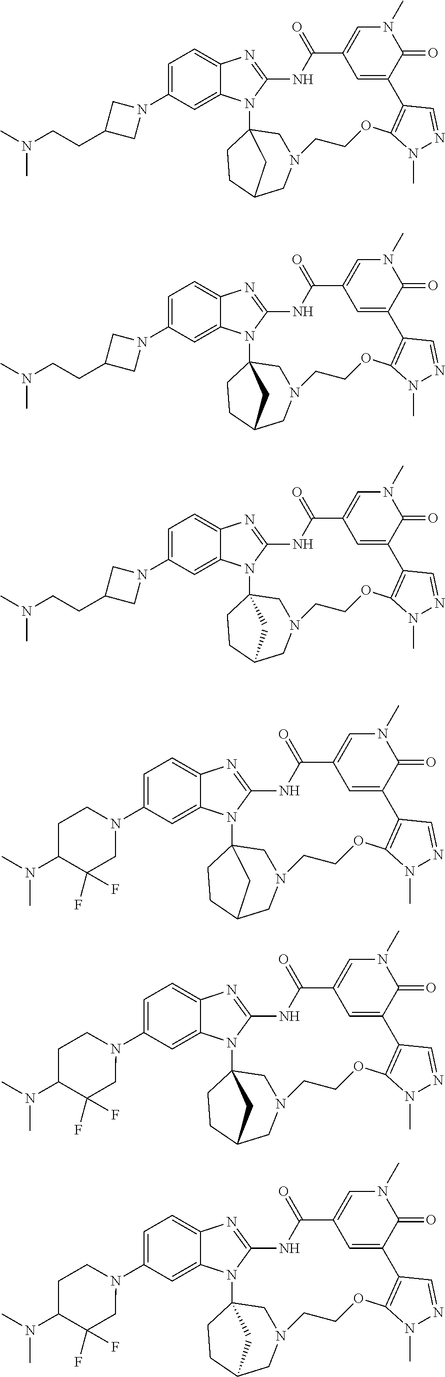

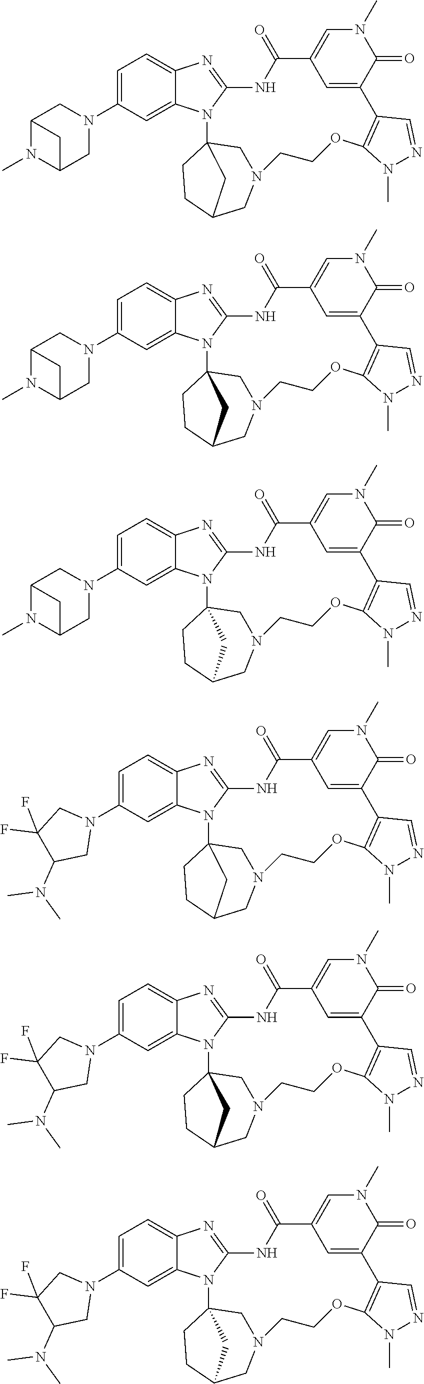

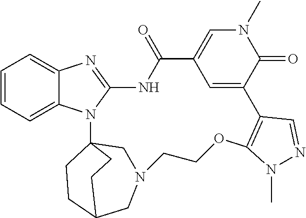 [SMILES: Cn1ncc2c1OCCN1CC3CCC(CC3)(C1)n1c(nc3ccccc31)NC(=O)c1cc-2c(=O)n(C)c1]

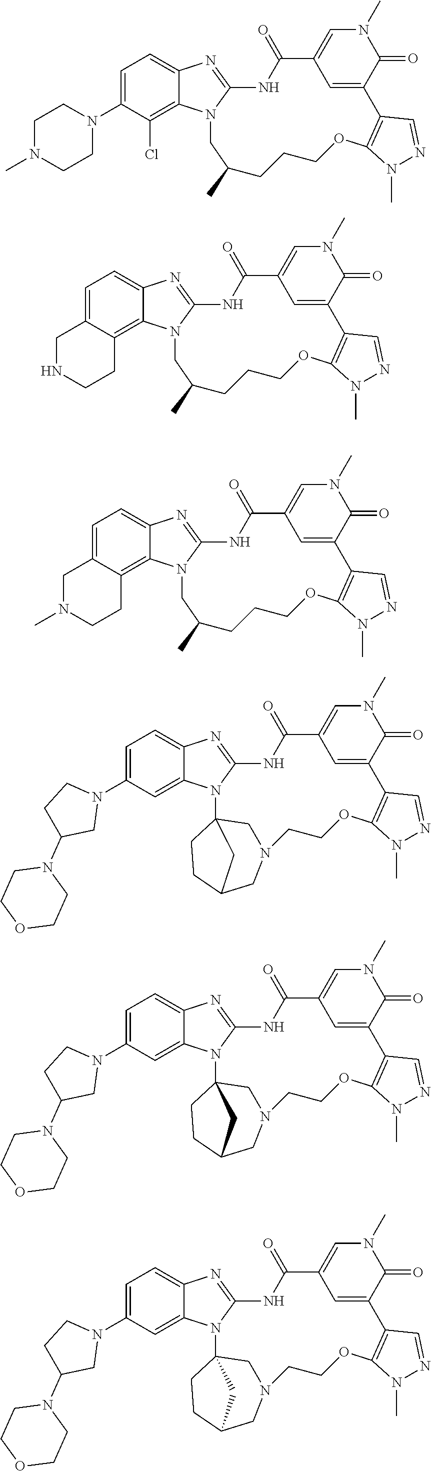

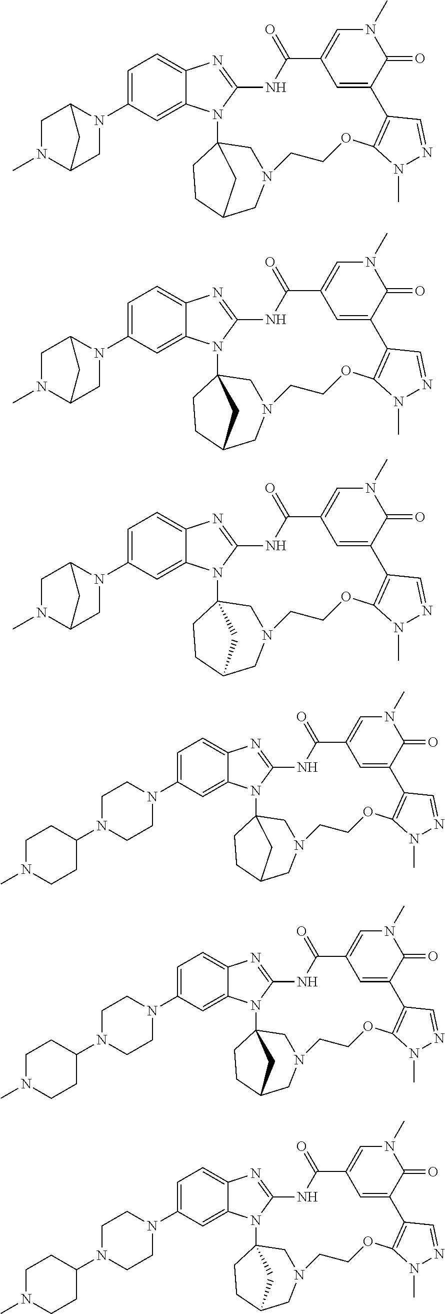

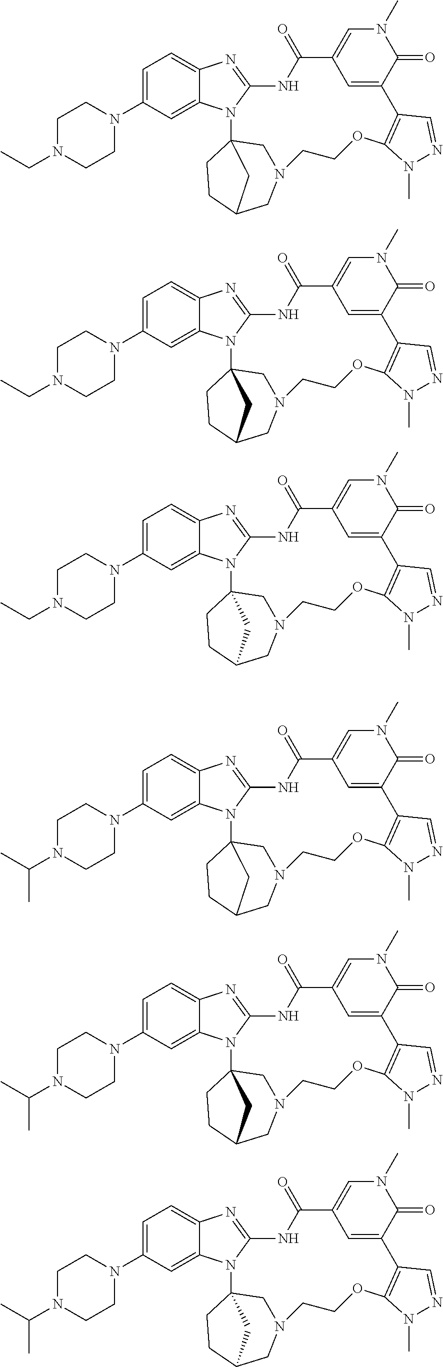

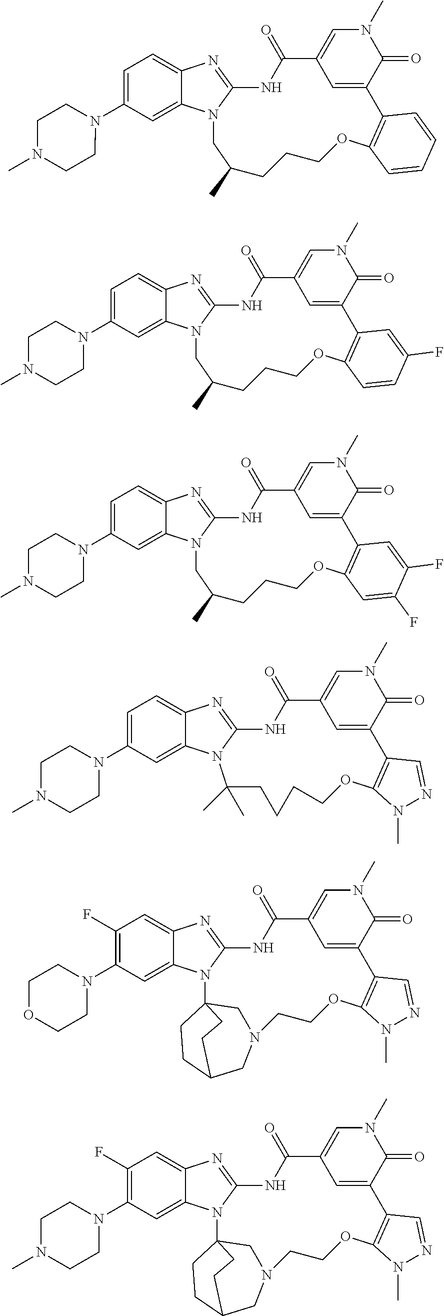

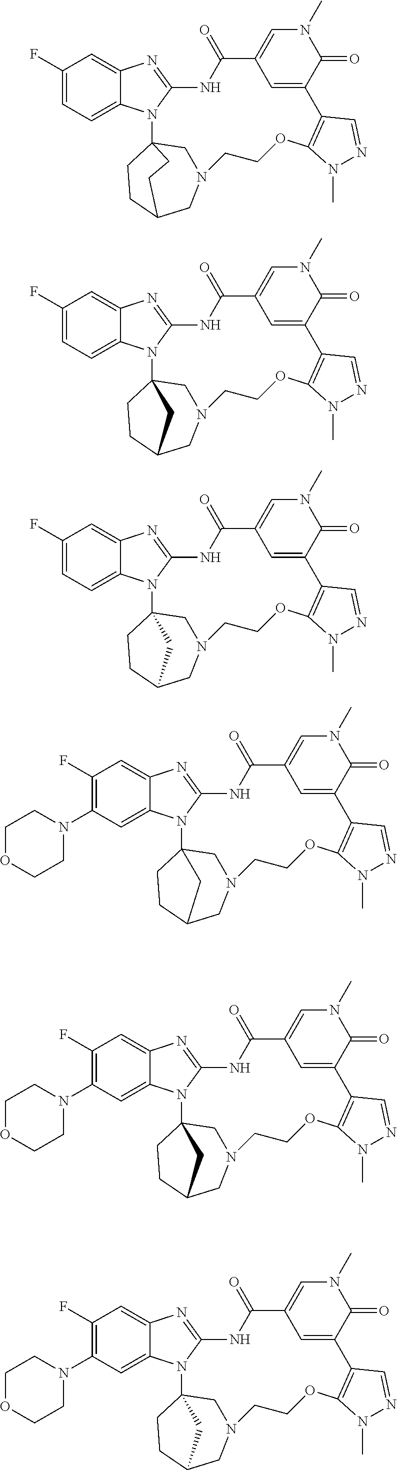

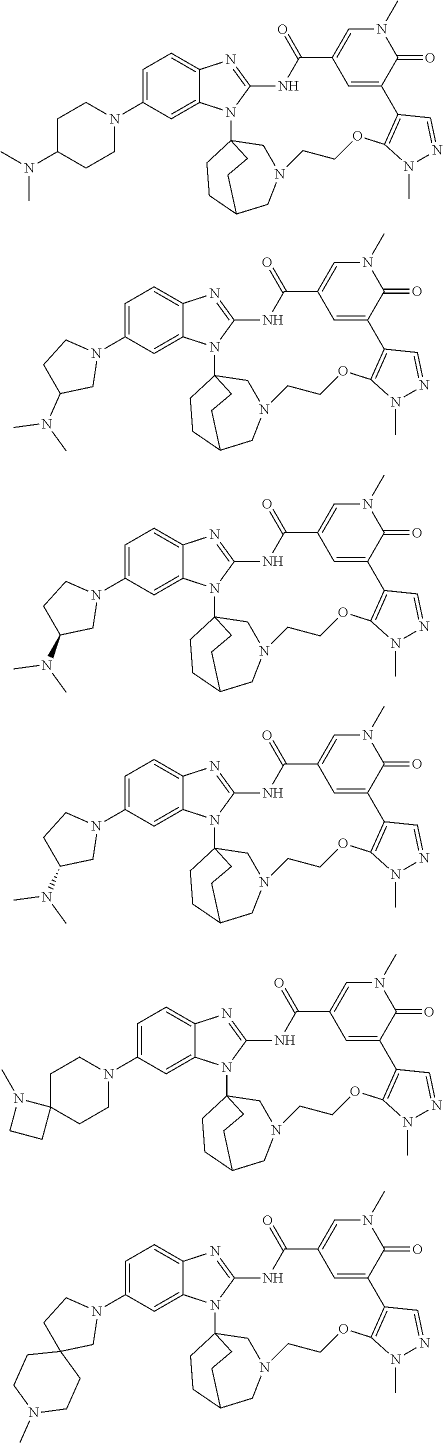

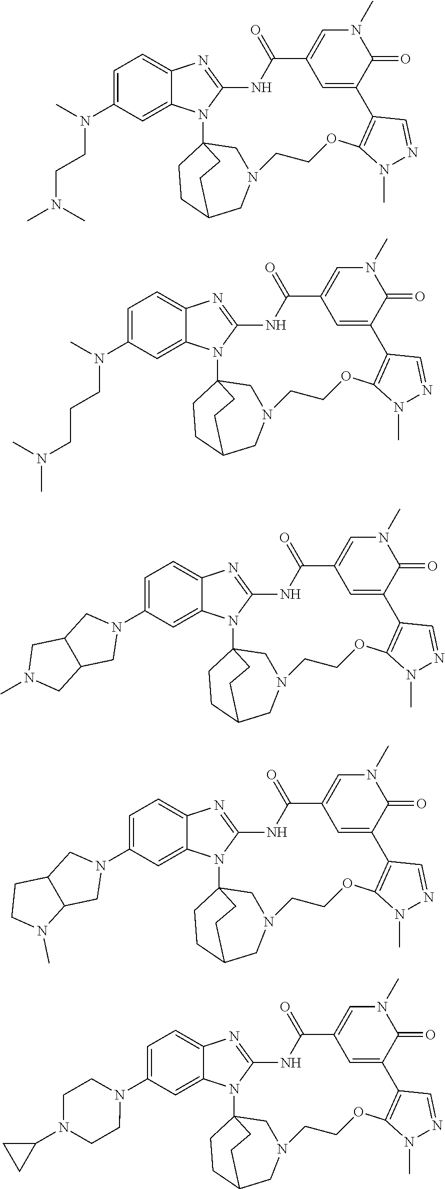

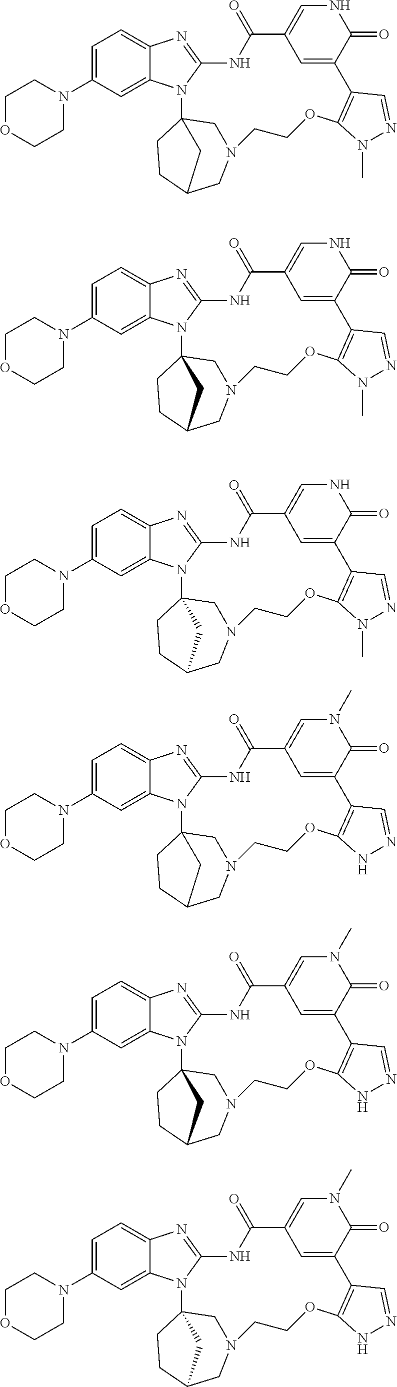

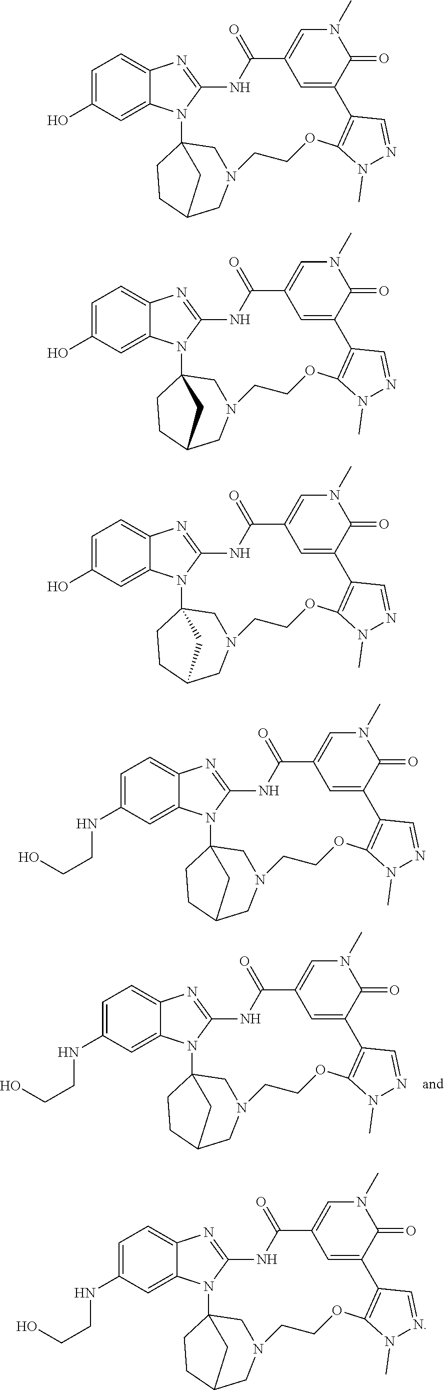

55. A pharmaceutical composition comprising the compound or a pharmaceutically acceptable salt thereof of any one of claims 1-54, and a pharmaceutically acceptable carrier.

56. A method of inhibiting EGFR activity in a subject in need thereof, comprising administering an effective amount of the compound or a pharmaceutically acceptable salt thereof of any one of claims 1-54, or the pharmaceutical composition of claim 55 to the subject.

57. A method of treating an EGFR-related disorder in a subject in need thereof, comprising administering to the subject an effective amount of the compound or a pharmaceutically acceptable salt thereof of any one of claims 1-54, or the pharmaceutical composition of claim 55 to the subject.

58. The method according to claim 57, wherein the EGFR-related disorder is autoimmune diseases or cancers.

59. The method according to claim 58, wherein the cancer is selected from the group consisting of lung cancer, brain cancers, colorectal cancer, bladder cancer, urothelial cancer, breast cancer, prostate cancer, ovarian cancer, head and neck cancer, pancreatic cancer, gastric cancer and mesothelioma, including metastasis (in particular brain metastasis) and the like.

60. The method according to any one of claims 57-59, wherein the compound is administered simultaneously, separately or sequentially with one or more additional therapeutic agents.

61. The method according to claim 60, wherein the one or more additional therapeutic agents are selected from the group consisting of EGFR TKIs, EGFR antibodies, MEK inhibitors, c-MET inhibitors, mitotic kinase inhibitors, immunotherapeutic agents, anti-angiogenic agents, apoptosis inducers, mTOR inhibitors, histone deacetylase inhibitors, IL6 inhibitors, and JAK inhibitors.
